SEQUENCE LISTING (3 sequences)

SEQ ID NO: 1 - protein, 6 aa, Homo sapiens
QCXXCG

SEQ ID NO: 2 - protein, 23 aa, Homo sapiens
QCXXCGXXXFXXXXXLXXHXXXH

SEQ ID NO: 3 - protein, 17 aa, Homo sapiens
QCXXCXXCGXXCXXXXX

(19) United States
(12) Patent Application Publication
(10) Pub. No.: US 2023/0372496 A1
(43) Pub. Date: Nov. 23, 2023

(54) TRICYCLIC HETEROBIFUNCTIONAL COMPOUNDS FOR DEGRADATION OF TARGETED PROTEINS

(71) Applicant: C4 Therapeutics, Inc., Watertown, MA (US)

(72) Inventors: Christopher G. Nasveschuk, Stoneham, MA (US); Corey Don Anderson, Brighton, MA (US); James A. Henderson, Weston, MA (US); Victoria Garza, San Antonio, TX (US); Yanke Liang, Belmont, MA (US); Moses Moustakim, Cambridge, MA (US); Katrina L. Jackson, Weston, MA (US); Martin Duplessis, Somerville, MA (US)

(73) Assignee: C4 Therapeutics, Inc., Watertown, MA (US)

(21) Appl. No.: 18/134,971

(22) Filed: Apr. 14, 2023

Related U.S. Application Data
(63) Continuation of application No. PCT/US21/55105, filed on Oct. 14, 2021.
(60) Provisional application No. 63/091,897, filed on Oct. 14, 2020.

(51) Int. Cl. A61K 47/55; A61K 47/54

(52) U.S. Cl. CPC A61K 47/55; A61K 47/545

(57) ABSTRACT

Heterobifunctional compounds for targeted protein degradation that include a tricyclic cereblon binder linked to an appropriate protein targeting ligand to degrade a targeted disease-mediating protein of interest are provided.

### CROSS-REFERENCE TO RELATED APPLICATIONS

This application is a continuation of International Patent Application No. PCT/US2021/055105, filed in the U.S. Receiving Office on Oct. 14, 2021, which claims benefit of and priority to U.S. Provisional Application No. 63/091,897, filed on Oct. 14, 2020, each of which is hereby incorporated by reference herein in its entirety.

### FIELD OF THE INVENTION

The disclosed invention provides catalytic pharmaceutical protein degraders that include a tricyclic cereblon binder linked to an appropriate protein targeting ligand to degrade a target disease-mediating protein of interest.

### INCORPORATION BY REFERENCE

The contents of the XML file named “16010-050WO1US1_ST26_2023-03-23.xml” which was created on Mar. 23, 2023, and is 3.67 KB in size, are hereby incorporated by reference in their entirety.

### BACKGROUND

Proteins are large, complex molecules that play many critical roles in the human body. Protein interactions control mechanisms involved with both healthy and disease states. A large number of diseases are caused by the mutation, alteration or overexpression of a protein, often leading to abnormal cellular proliferation or other dysfunction.

The human body has a highly conserved homeostasis system which maintains a stable equilibrium of proteins. It relies on elaborate protein degradation machinery to identify and break down proteins into their component amino acids. This process is mediated in part by “E3 ligases” which act as quality control inspectors by identifying proteins that are old, damaged, misfolded or otherwise ready for degradation. The E3 ligase attaches a series of molecular tags called ubiquitins to the protein in a process called ubiquitination. Once the protein is polyubiquitinated, it is released by the E3 ligase and quickly recognized by the proteasome, which is the cell's recycling plant. The proteasome degrades the ubiquitinated protein into its amino acids for recycling into new proteins.

This protein degradation system is sometimes referred to as the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection. Inadequate or defective proteasomal degradation has been linked to a variety of clinical disorders including abnormal cellular proliferation, including cancer, neurodegenerative diseases such as Alzheimer's disease, Parkinson's disease, Huntington's disease, muscular dystrophy and cardiovascular disease.

Historically, disease mediating proteins were targeted for medical therapy using inhibitors that fit into an enzyme pocket and interfered with protein activity, or by otherwise binding to the protein to disrupt its activity. However, a number of proteins are “undruggable” because they are not enzymes, and do not have an active pocket or are not susceptible to binding with an interfering molecule in vivo. Inhibition mechanisms often require high doses of drug for adequate, sustained target occupancy. Since the pharmacological effect is driven by drug exposure, the overall timing and duration of drug action is dependent on drug absorption, distribution and elimination. These drug levels can be hard to achieve and can cause significant off-target effects. The inhibition approach which requires the identification of proteins with specific active sites and compounds that inhibit the sites in a well-behaved manner is difficult.

Recently, efforts have been made to capitalize on the body's proteasomal protein degradation system to degrade instead of inhibit disease-mediating proteins.

Patent applications filed by C4 Therapeutics, Inc., that describe compounds capable of binding to an E3 ubiquitin ligase and a target protein for degradation include: WO 2021/178920 titled “Compounds for Targeted Degradation of BRD9”; WO 2021/127561 titled “Isoindolinone and Indazole Compounds for the Degradation of EGFR”; WO 2021/086785 titled “Bifunctional Compounds”; WO 2021/083949 titled “Bifunctional Compounds for the Treatment of Cancer”; WO 2020/132561 titled “Targeted Protein Degradation”; WO 2019/236483 titled “Spirocyclic Compounds”; WO 2020/051235 titled “Compounds for the Degradation of BRD9 or MTH1”; WO 2019/191112 titled “Cereblon Binders for the Degradation of Ikaros”; WO 2019/204354 titled “Spirocyclic Compounds”; WO 2019/099868 titled “Degraders and Degrons for Targeted Protein Degradation”; WO 2018/237026 titled “N/O-Linked Degrons and Degronimers for Protein Degradation”; WO 2017/197051 titled “Amine-Linked C3-Glutarimide Degronimers for Target Protein Degradation”; WO 2017/197055 titled “Heterocyclic Degronimers for Target Protein Degradation”; WO 2017/197036 titled “Spirocyclic Degronimers for Target Protein Degradation”; WO 2017/197046 titled “C3-Carbon Linked Glutarimide Degronimers for Target Protein Degradation”; and WO 2017/197056 titled “Bromodomain Targeting Degronimers for Target Protein Degradation.”

WO 2020/210630 filed by C4 Therapeutics Inc. describes tricyclic compounds. WO 2021/127586 filed by Calico Life Sciences LLC and AbbVie Inc. describes PTPN1 and PTPN2 ligands covalently bound to various cereblon ligands.

Additional examples of protein degradation applications include WO2021/041664, WO2021/143822, WO2021/143816, WO2020/010227, WO2020/006262, and WO2019/148055.

Despite these efforts there remains a need for new compounds and pharmaceutical compositions that degrade disease-mediating proteins, methods for their use and processes for their preparation.

### SUMMARY OF THE INVENTION

Compounds and their uses and manufacture are provided that degrade a disease-mediating Target Protein via the ubiquitin proteasome pathway (UPP) to treat a disease in a host, typically a human, that is responsive to the degradation of the protein. The invention provides compounds of general Formula I, Formula II, or Formula III, or a pharmaceutically acceptable salt thereof that include a Targeting Ligand that binds to a Target Protein, an E3 Ligase binding portion (Tricyclic Cereblon Ligand), a Linker that covalently links the Targeting Ligand to a Spacer, and a Spacer that covalently links the Linker to the E3 Ligase binding portion.

A compound of the present invention provided herein or its pharmaceutically acceptable salt and/or its pharmaceutically acceptable composition thereof can be used to treat a disorder which is mediated by a Target Protein. The Target Protein is typically a mutated, altered or overexpressed protein wherein the mutation, alteration or overexpression converts its normal function into a dysfunction which causes or contributes to disease. In some aspects, the disease is an abnormal cellular proliferation such as cancer or a tumor. In some embodiments a method to treat a patient with a disorder mediated by a Target Protein is provided that includes administering an effective amount of one or more compounds as described herein, or a pharmaceutically acceptable salt thereof, to the patient, typically a human, optionally in a pharmaceutically acceptable composition. In some embodiments, the tricyclic cereblon binding heterobifunctional compound is administered to a host, typically a human, in need thereof in combination with another pharmaceutical or a biologic agent, which may be standard of care for the disease to be treated.

The tricyclic cereblon binding heterobifunctional compounds provided herein are catalytic. The targeted protein degradation mediated by the compound typically occurs rapidly, on the order of milliseconds from initial target-ligase encounter to poly-ubiquitination and release for degradation by the proteasome. Once the targeted protein degradation process occurs for one molecule of a target protein, the degrader is released and the process is repeated with the same degrader molecule. This recursive process of binding the target protein, ternary complex formation with the E3 ligase, ubiquitination and release for degradation can occur thousands of times with a single degrader molecule.

In one aspect, the tricyclic cereblon binding heterocyclic degraders described herein are orally bioavailable and can be provided in an effective amount in a convenient solid dosage form, including but not limited to a pill, tablet, gelcap or liquid. Alternatively, the degrader can be administered parenterally, including via intravenous delivery, or topically, or otherwise as described further herein.

In one aspect, a compound is provided of Formula I:

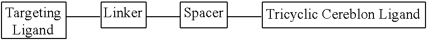

or a pharmaceutically acceptable salt, N-oxide, isotopic derivative, or prodrug thereof, optionally in a pharmaceutically acceptable carrier to form a composition.

The Tricyclic Cereblon Ligand is selected from one of the following moieties, wherein the bracketed bond indicates that the tricyclic moiety is attached to the Spacer/Linker via a covalent bond on Cycle-A, Cycle-B, Cycle-C or Cycle-D as relevant in a manner that achieves the desired potency and catalytic degradation profile.

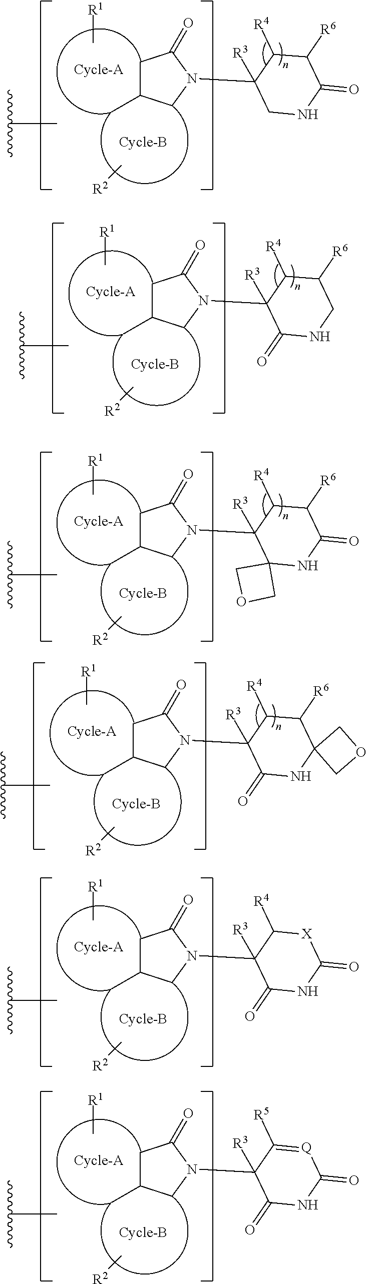

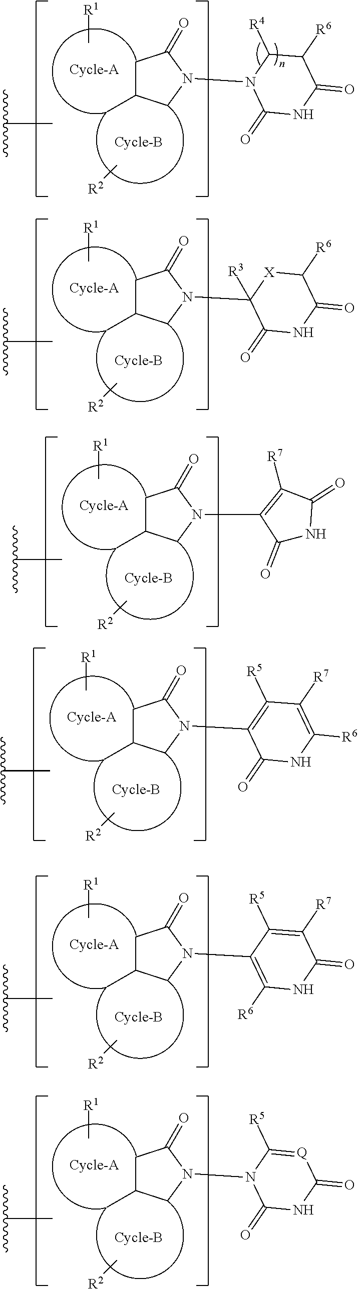

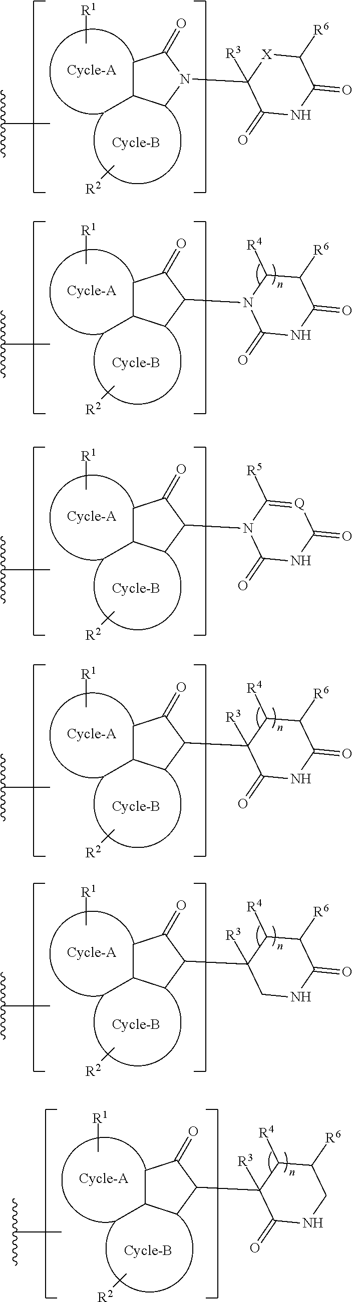

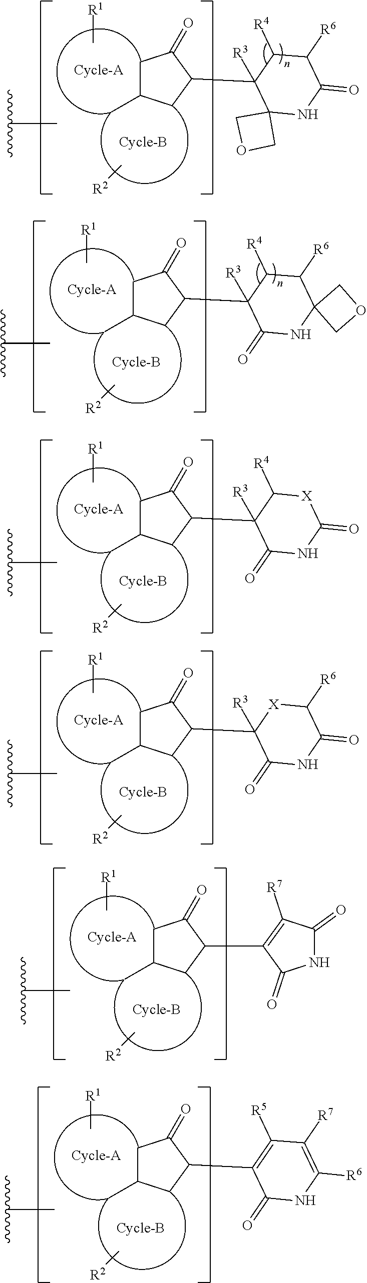

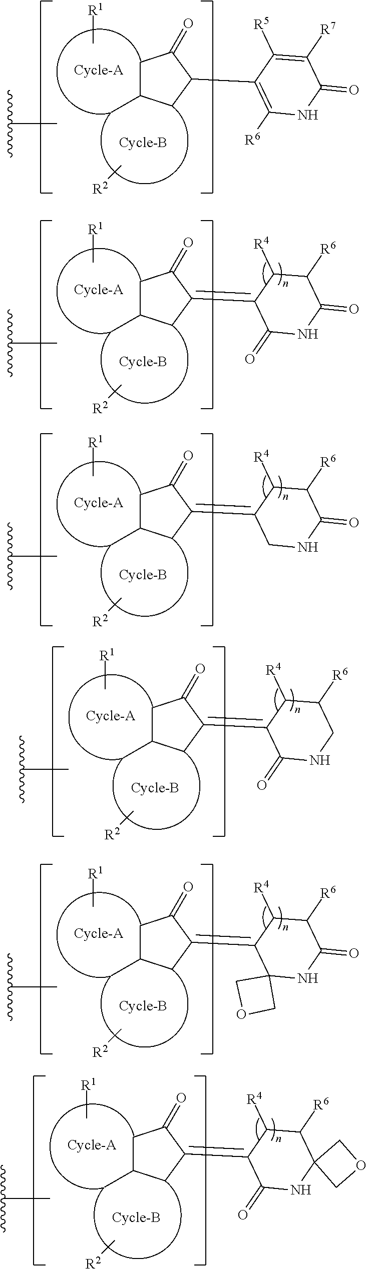

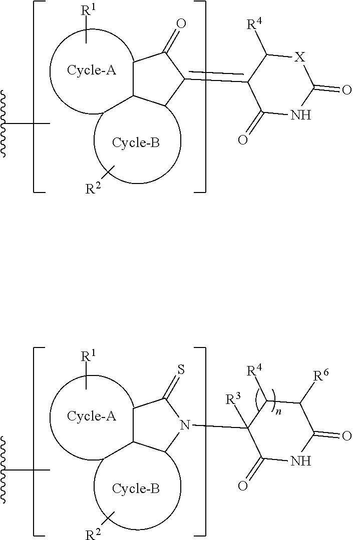

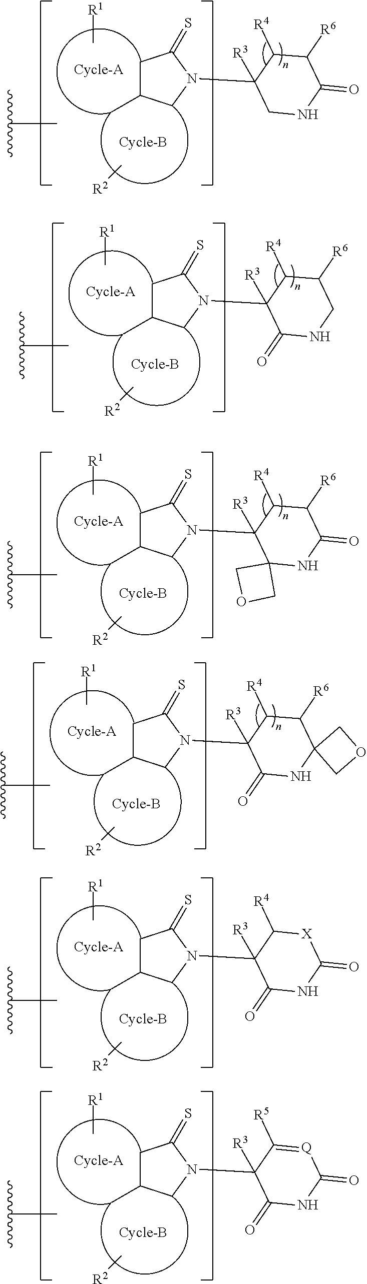

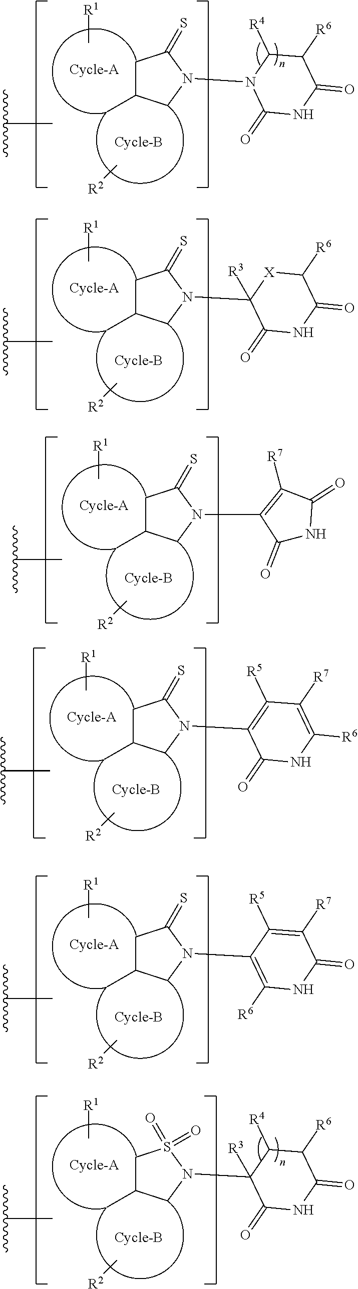

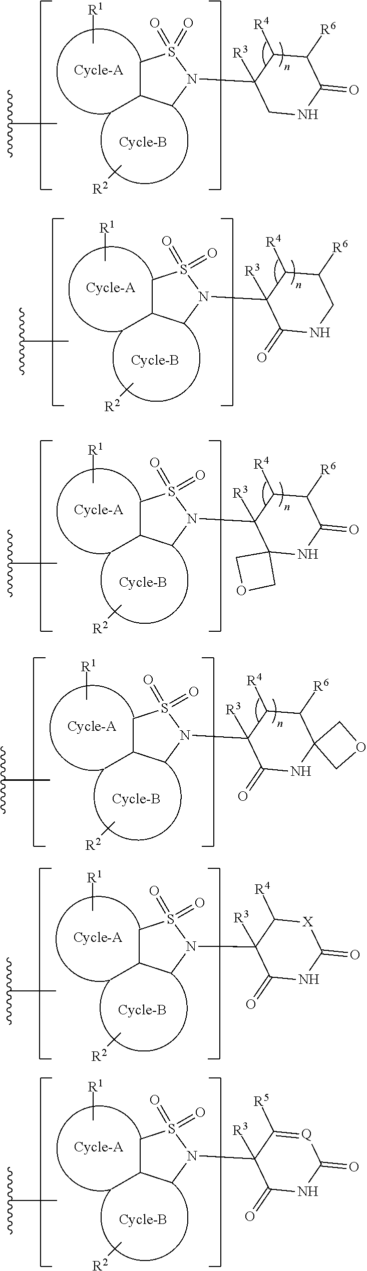

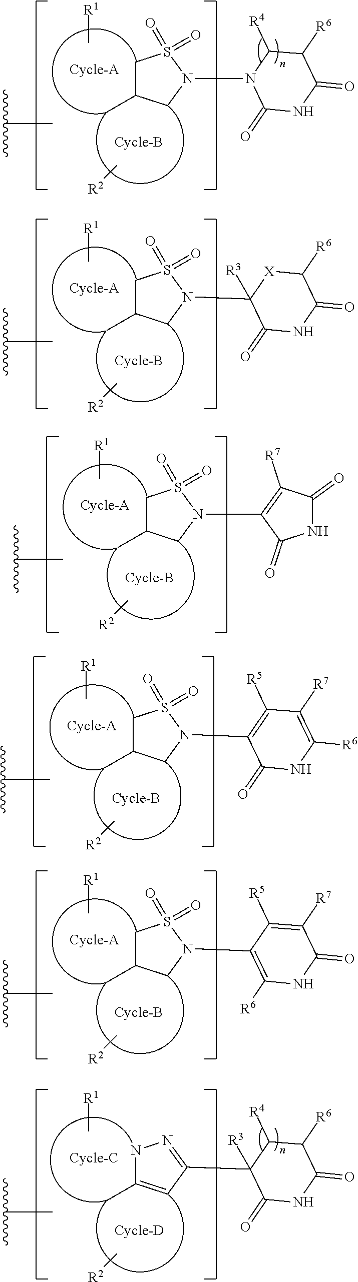

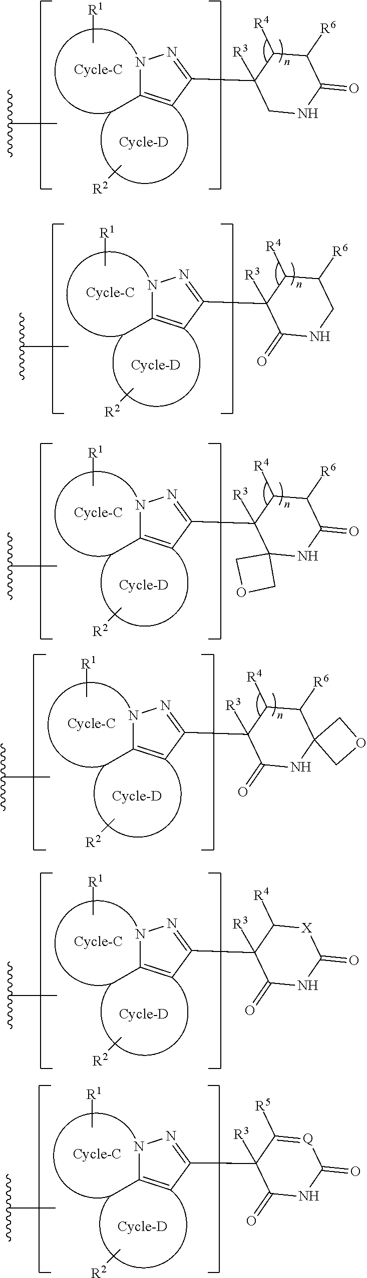

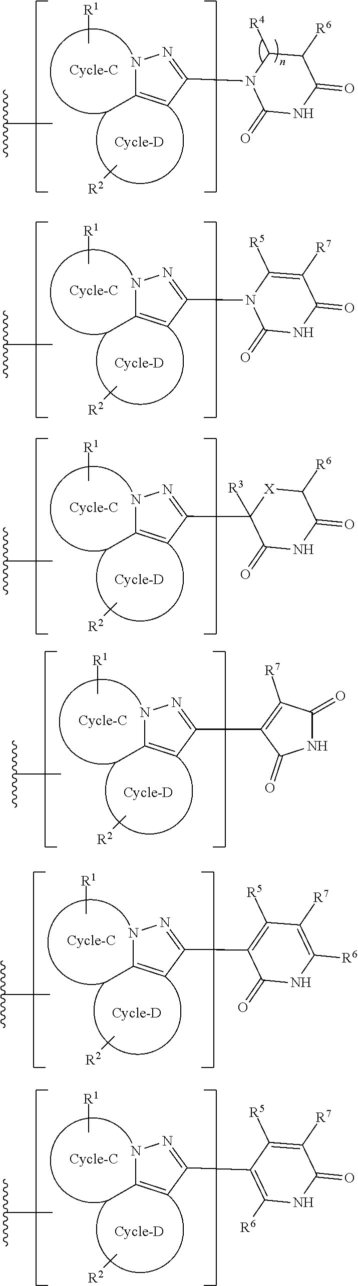

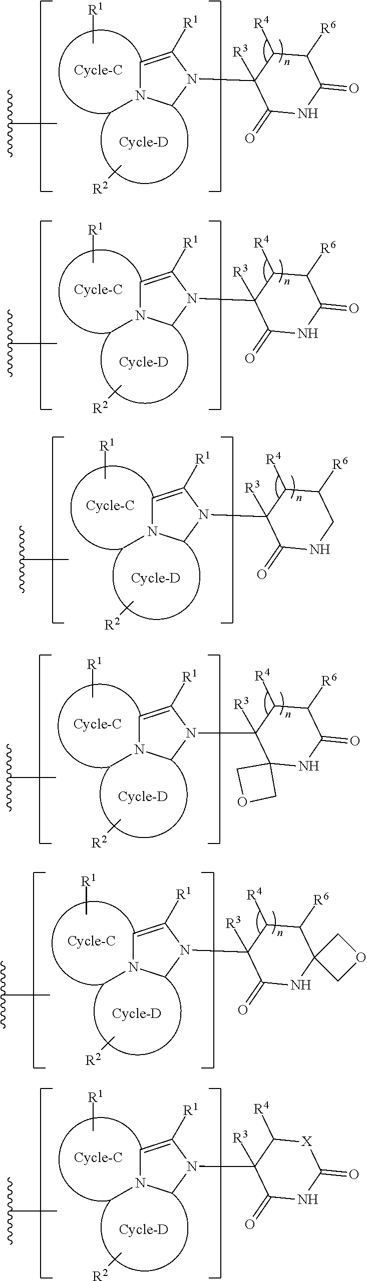

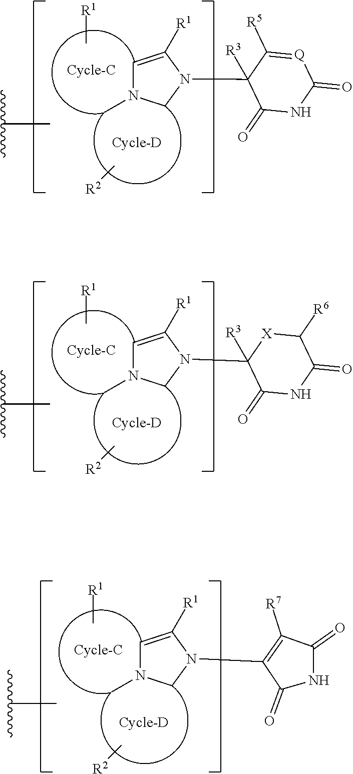

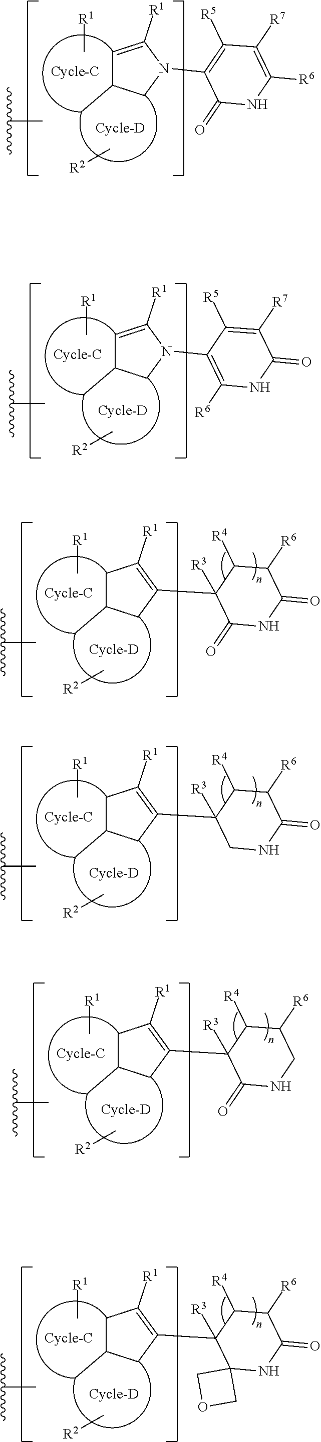

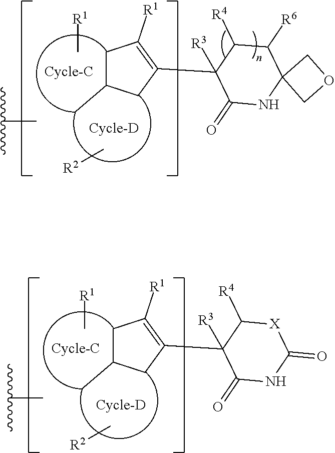

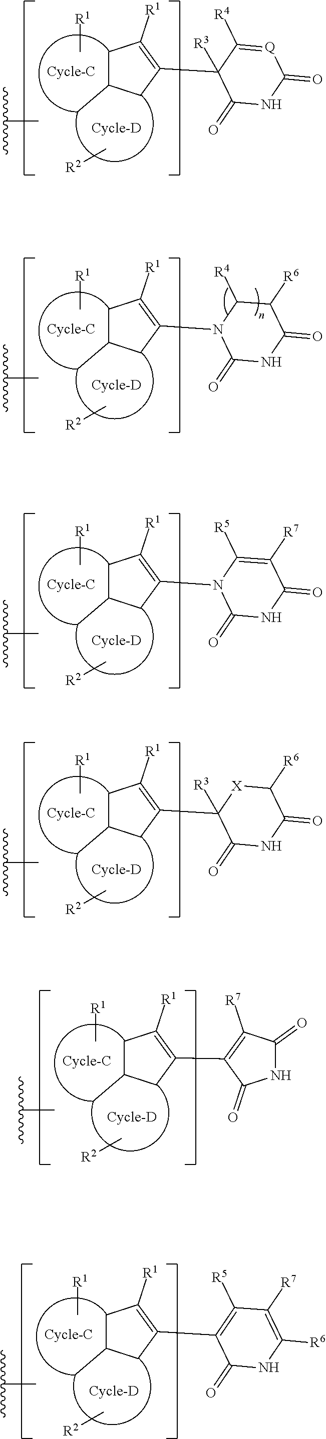

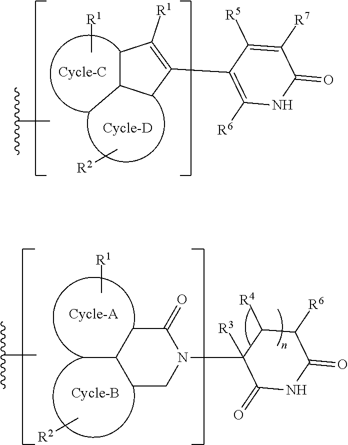

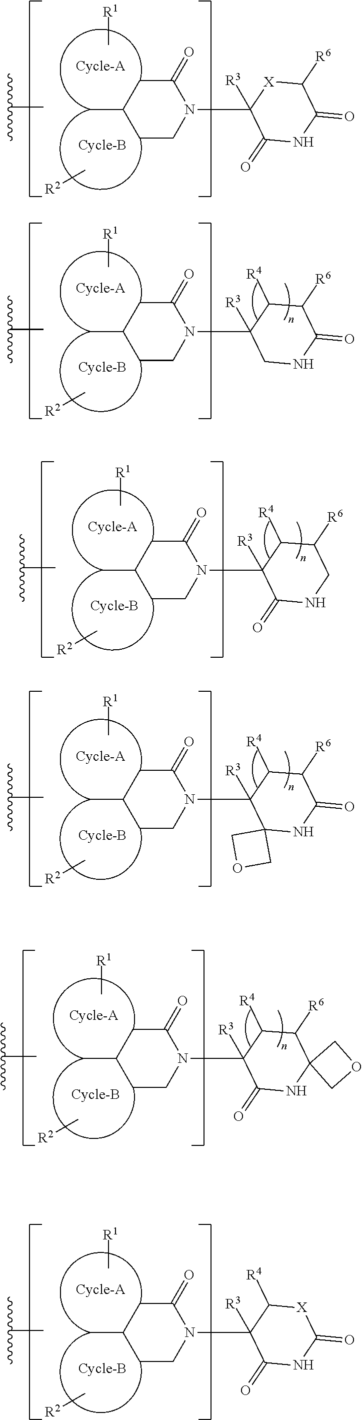

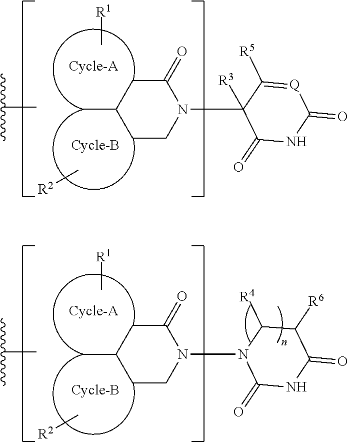

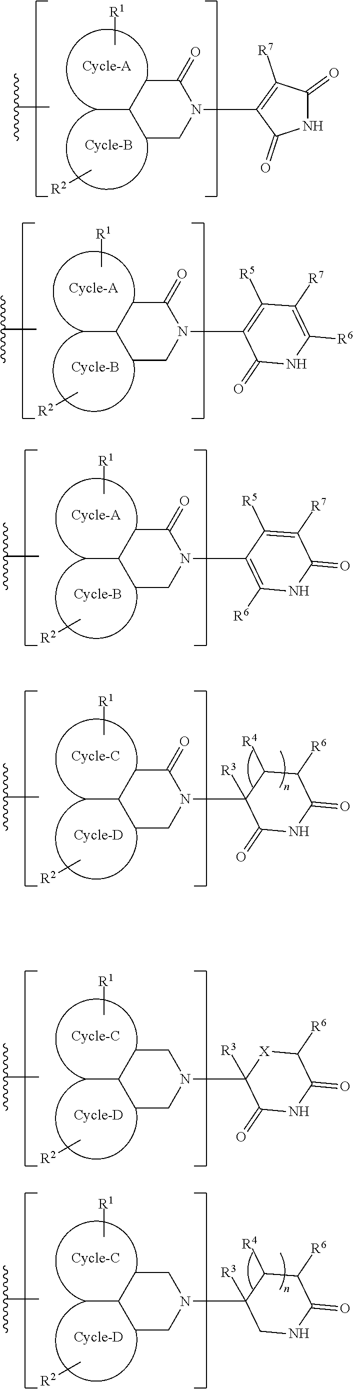

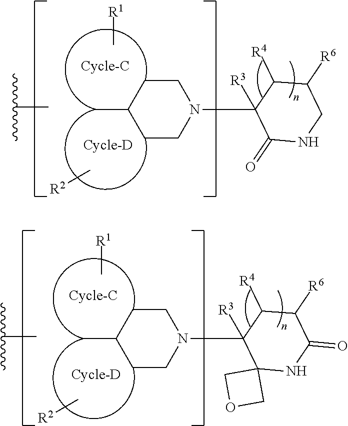

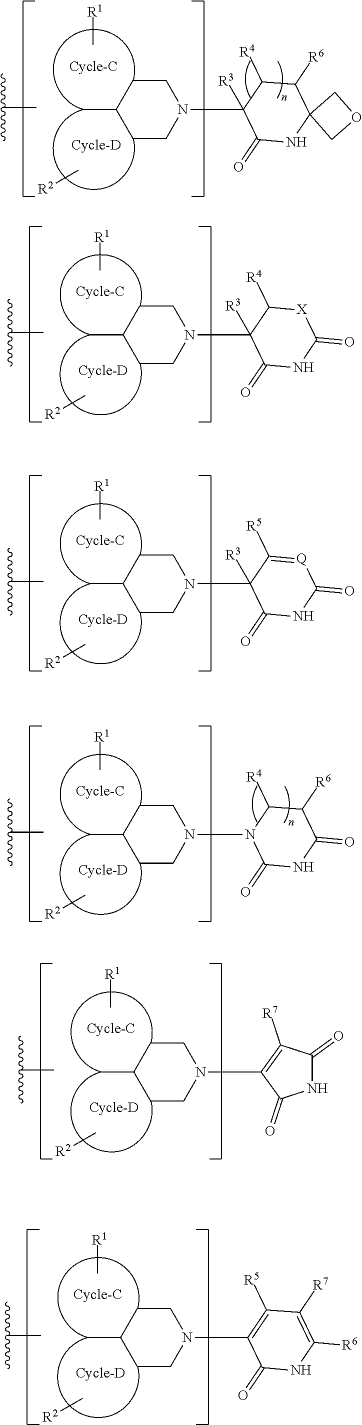

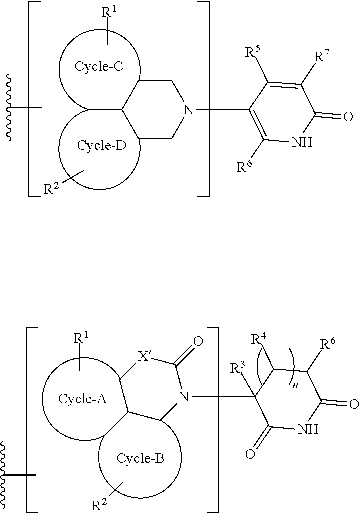

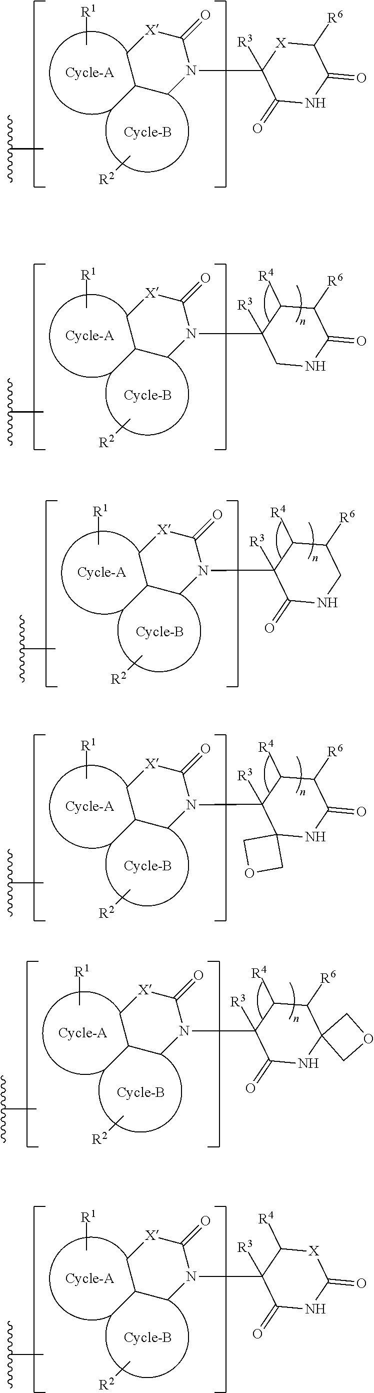

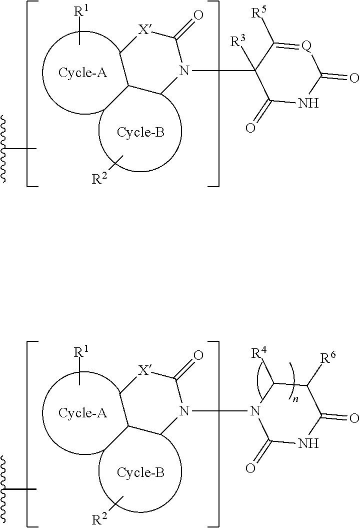

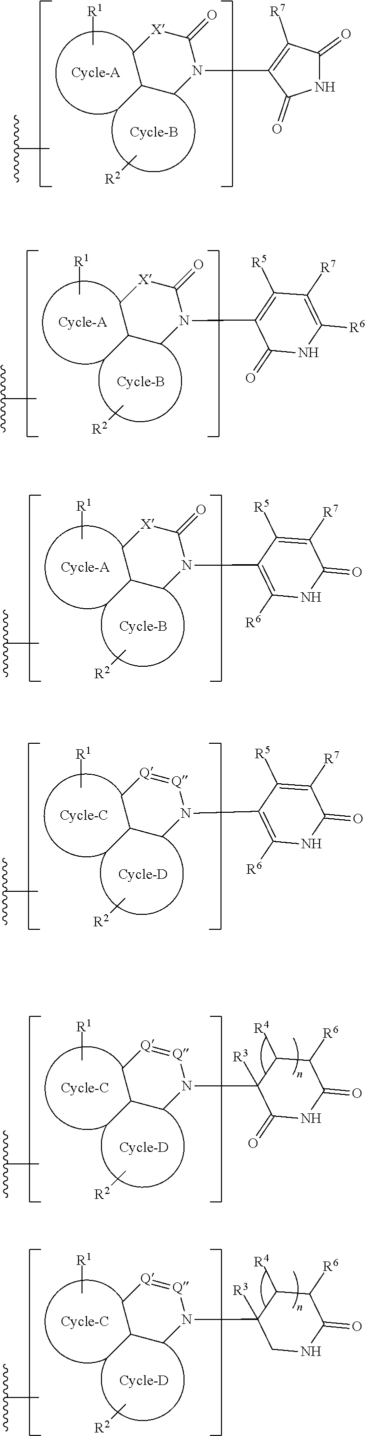

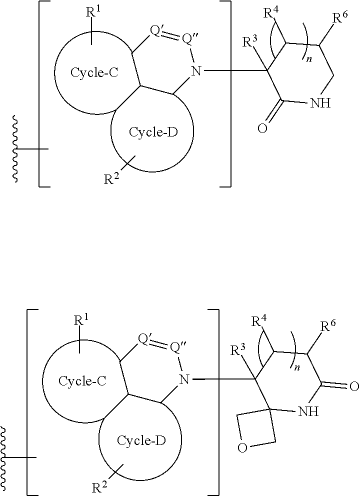

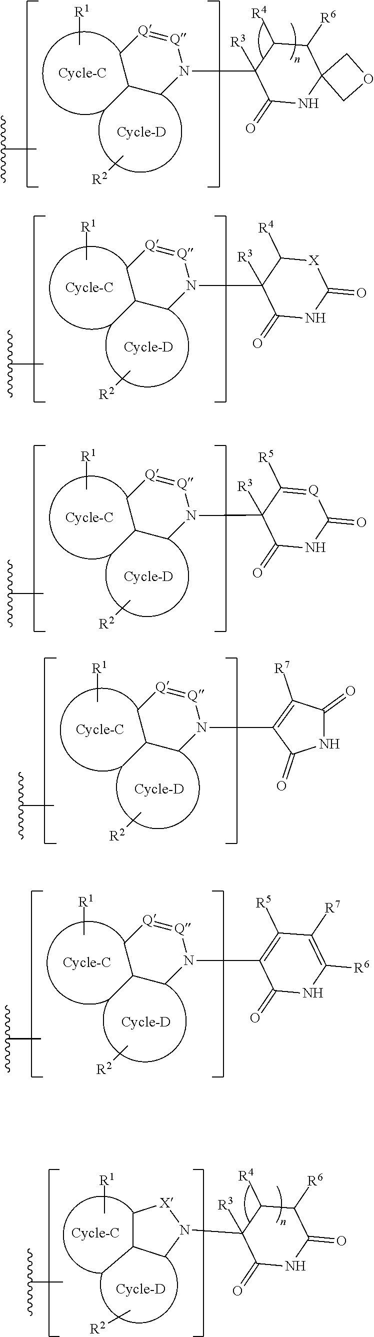

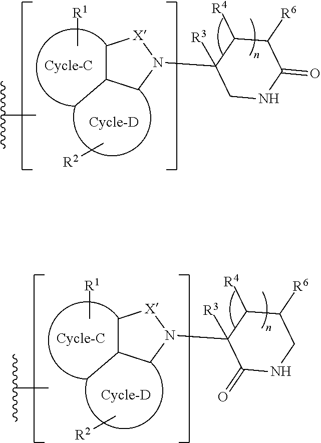

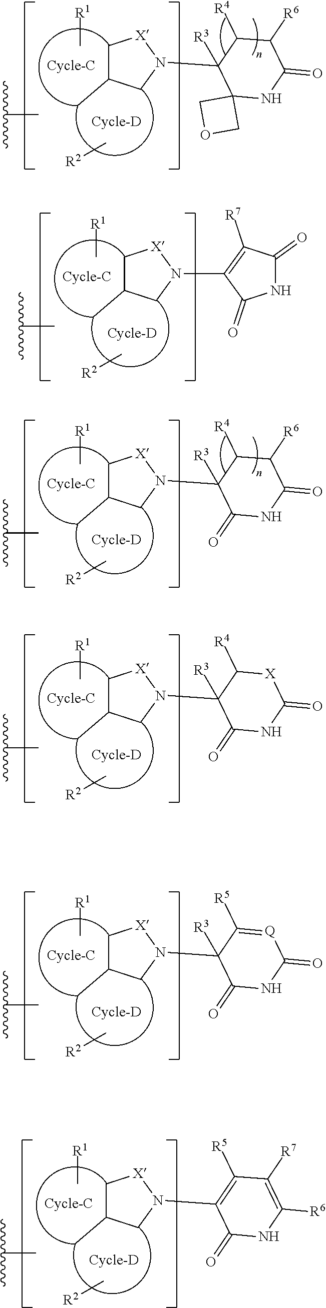

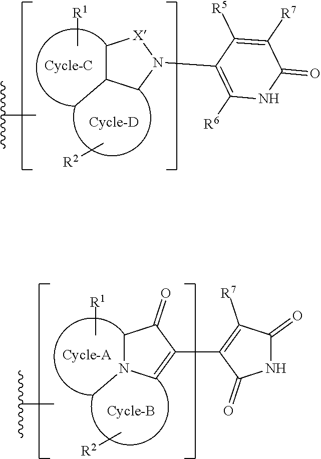

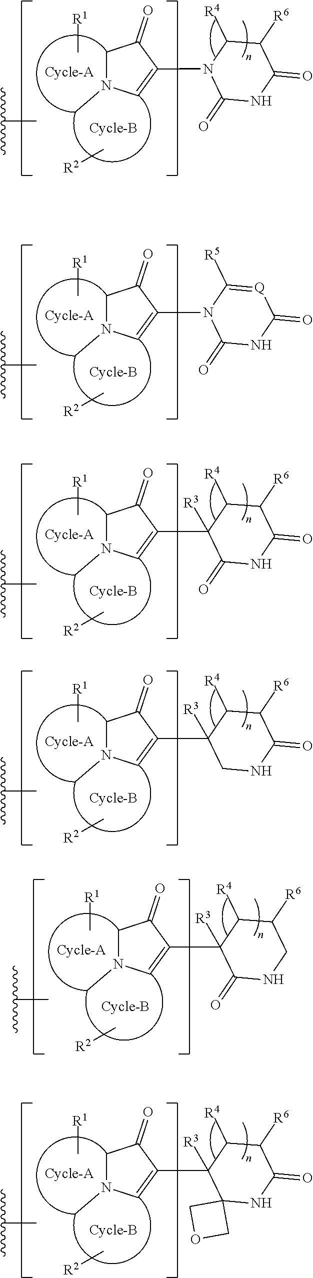

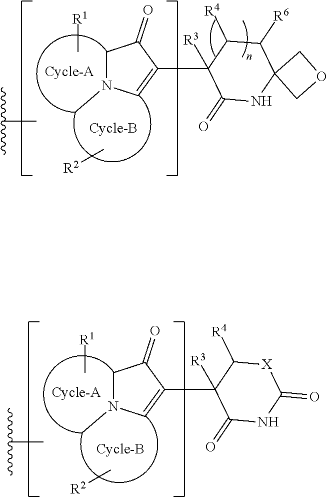

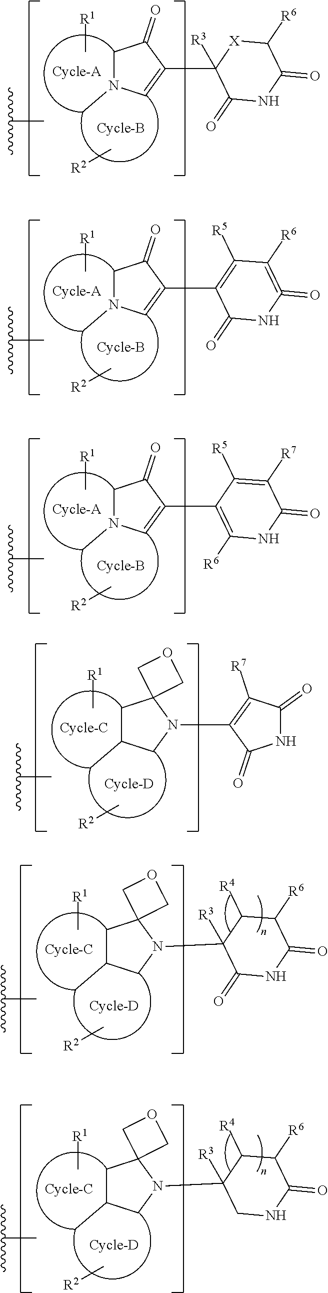

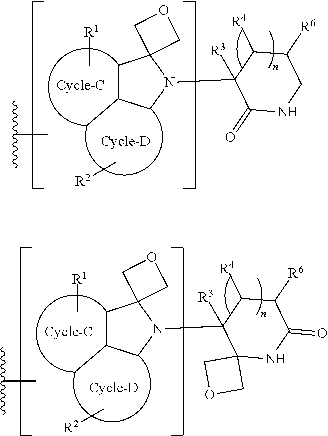

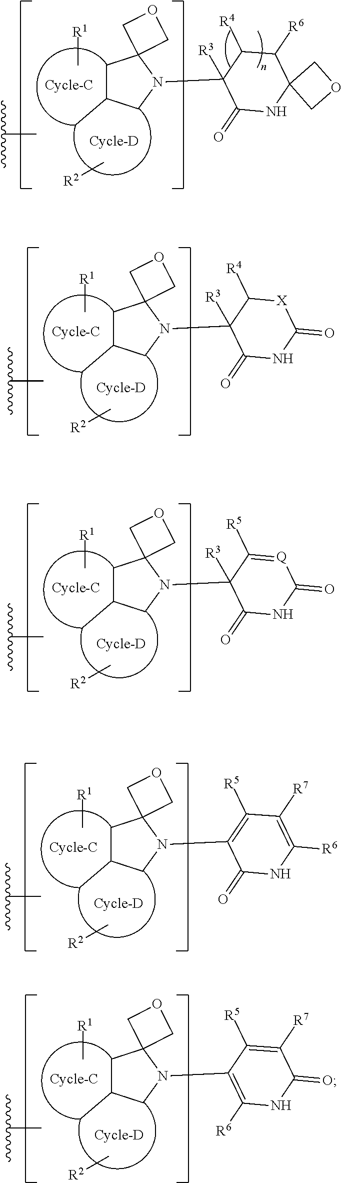

n is 0, 1, or 2;
X is NR¹⁰, NR^{6′}, O, or S;
X′ is NR¹⁰, O, CH₂, or S;
Q is CR⁷ or N;
Q′ and Q″ are independently selected from CR¹ and N.
Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1, 2, or 3 substituents independently selected from R¹ as allowed by valence.
Cycle-B is a fused ring selected from phenyl, 5- or 6-membered heteroaryl, 5- to 8-membered heterocycle, 5- to 8-membered cycloalkyl, or 5- to 8-membered cycloalkenyl, wherein Cycle-B is optionally substituted with 1, 2, or 3 substituents independently selected from R² as allowed by valence.

In certain embodiments Cycle-A is a fused ring selected from phenyl, 5- or 6-membered heteroaryl, 5- to 6-membered heterocycle, 5- to 6-membered cycloalkyl, or 5- to 6-membered cycloalkenyl, wherein Cycle-A is optionally substituted with 1, 2, or 3 substituents independently selected from R¹ as allowed by valence.

In certain embodiments Cycle-B is a fused ring selected from phenyl, 5- or 6-membered heteroaryl, 5- to 6-membered heterocycle, 5- to 6-membered cycloalkyl, or 5- to 6-membered cycloalkenyl, wherein Cycle-B is optionally substituted with 1, 2, or 3 substituents independently selected from R² as allowed by valence.
Cycle-C is a fused ring selected from phenyl, 5- or 6-membered heteroaryl, 5- to 6-membered heterocycle, 5- to 6-membered cycloalkyl, or 5- to 6-membered cycloalkenyl, wherein each Cycle-C is optionally substituted with 1, 2, or 3 substituents independently selected from R¹ as allowed by valence.
Cycle-D is a fused ring selected from phenyl, 5- or 6-membered heteroaryl, 5 to 6-membered heterocycle, 5- to 6-membered cycloalkyl, or 5- to 6-membered cycloalkenyl, wherein each Cycle-D is optionally substituted with 1, 2, or 3 substituents independently selected from R² as allowed by valence.
R¹ and R² are independently at each instance selected from hydrogen, alkyl, halogen, haloalkyl, —OR¹⁰, —SR¹⁰, —S(O)R¹², —SO₂R¹², —NR¹⁰R¹¹, cyano, nitro, heteroaryl, aryl, and heterocycle; or alternatively, if allowed by valence and stability, R¹ or R² may be a divalent moiety such as ═O, ═S, or ═NR⁴¹; and wherein an R¹ group may optionally be combined with another R¹ group or an R² group to form a fused cycle or bicycle which may bridge Cycle-A and Cycle-B or Cycle-C and Cycle-D, as appropriate and desired.
R³ is hydrogen, alkyl, halogen, or haloalkyl;
or R³ and R⁶ are combined to form a 1 or 2 carbon attachment, for example when R³ and R⁶ form a 1 carbon attachment

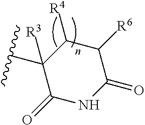

is

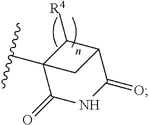

or R³ and R⁴ are combined to form a 1, 2, 3, or 4 carbon attachment, for example when R³ and R⁴ form a 1 carbon attachment

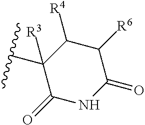

is

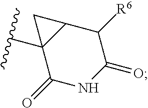

or R³ and an R⁴ group adjacent to R³ are combined to form a double bond.
R⁴ and R⁵ are independently selected from hydrogen, alkyl, halogen, and haloalkyl;
R⁶ and R⁷ are independently selected from hydrogen, alkyl, halogen, haloalkyl, —OR¹⁰, —SR¹⁰, —S(O)R¹², —SO₂R¹², and —NR¹⁰R¹¹;
R^{6′} is hydrogen, alkyl, or haloalkyl;
or R³ and R^{6′} are combined to form a 1 or 2 carbon attachment.
R¹⁰ and R¹¹ are independently selected from hydrogen, alkyl, haloalkyl, heterocycle, aryl, heteroaryl, —C(O)R¹², —S(O)R¹², and —SO₂R¹²;
each R¹² is independently selected from hydrogen, alkyl, haloalkyl, heterocycle, aryl, heteroaryl, —NR¹³R¹⁴, and OR¹³;
and each instance of R¹³ and R¹⁴ is independently selected from hydrogen, alkyl, and haloalkyl.

Spacer is a bivalent connecting moiety which may be of the structure:

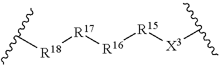

X³ is a bivalent moiety selected from bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂— and —S—; or can be arylalkyl, heterocyclealkyl or heteroarylalkyl (in either direction), each of which heterocycle, aryl, heteroaryl, and bicycle may be substituted with 1, 2, 3, or 4 substituents independently selected from R⁴⁰;
R¹⁵, R¹⁶, R¹⁷, and R¹⁸ are independently at each occurrence selected from the group consisting of a bond, alkyl (which in certain embodiments is a carbocycle), —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, arylalkyl, heterocyclealkyl, and heteroarylalkyl; each of which is optionally substituted with 1, 2, 3, or 4 substituents independently selected from R⁴⁰;
wherein X³ and R¹⁵⁻¹⁸ together are a stable moiety covalently connecting the Tricyclic Cereblon Ligand to the Linker, and wherein in certain embodiments Spacer is a covalent bond;
R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, oxo, and cycloalkyl;
R⁴¹ is aliphatic (including alkyl), aryl, heteroaryl, or hydrogen;
Targeting Ligand is a moiety that binds to a Target Protein and is covalently linked to the Tricyclic Cereblon Ligand through the Linker-Spacer;
Target Protein is a selected protein that causes or contributes to the disease to be treated in vivo;
Linker is a bivalent linking group, for example a bivalent linking group of Formula LI.

In certain embodiments Linker is of formula:

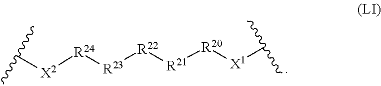

wherein,
X¹ and X² are independently at each occurrence selected from bond, heterocycle, aryl, heteroaryl, bicycle, alkyl, aliphatic, heteroaliphatic, —NR²⁷—, —CR⁴⁰R⁴¹—, —O—, —C(O)—, —C(NR²⁷)—, —C(S)—, —S(O)—, —S(O)₂— and —S—; each of which heterocycle, aryl, heteroaryl, and bicycle is optionally substituted with 1, 2, 3, or 4 substituents independently selected from R⁴⁰;
R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, oxyalkylene, —C(R⁴OR⁴⁰)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, and carbocycle; each of which is optionally substituted with 1, 2, 3, or 4 substituents independently selected from R⁴⁰;
R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, oxo, and cycloalkyl; and
R⁴¹ is aliphatic, aryl, heteroaryl, or hydrogen.

In certain aspects, a compound is provided of Formula II:

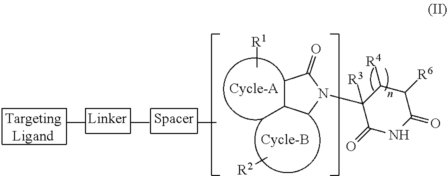

or a pharmaceutically acceptable salt, N-oxide, isotopic derivative, or prodrug thereof, optionally in a pharmaceutically acceptable carrier to form a composition;
wherein for Formula II:
Targeting Ligand is a moiety that binds to a Target Protein and is covalently linked to the Tricyclic Cereblon Ligand through the Linker-Spacer wherein the Targeting Ligand does not include the following substructure

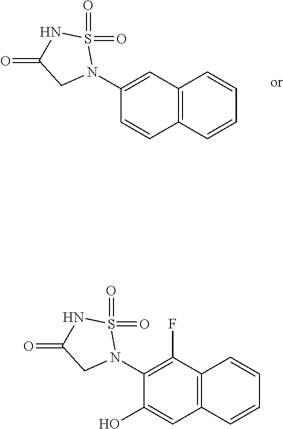 [SMILES: O=C1CN(c2ccc3ccccc3c2)S(=O)(=O)N1 ; O=C1CN(c2c(O)cc3ccccc3c2F)S(=O)(=O)N1]

Target Protein is a selected protein that causes or contributes to the disease to be treated in vivo wherein Target Protein is not a PTPase (e.g., PTPN1 or PTPN2),
and all other variables are as defined in Formula I or the embodiments described herein.

In certain aspects, a compound is provided of Formula III:

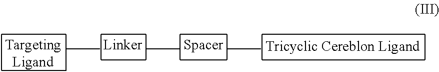

or a pharmaceutically acceptable salt, N-oxide, isotopic derivative, or prodrug thereof, optionally in a pharmaceutically acceptable carrier to form a composition.
wherein for Formula III:
Tricyclic Cereblon Ligand is selected from:

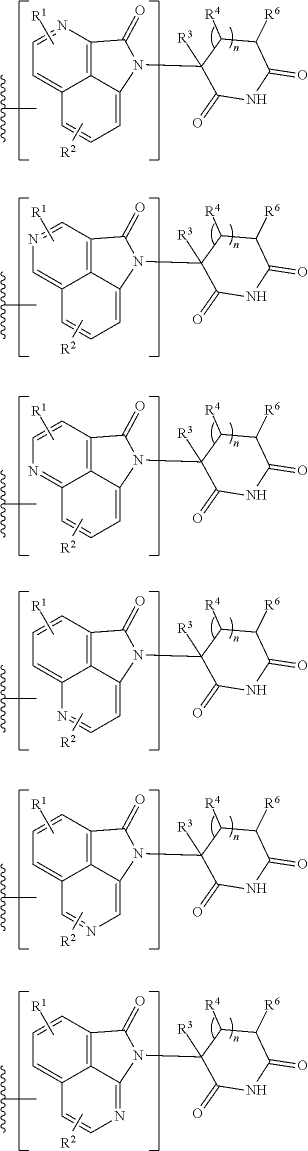

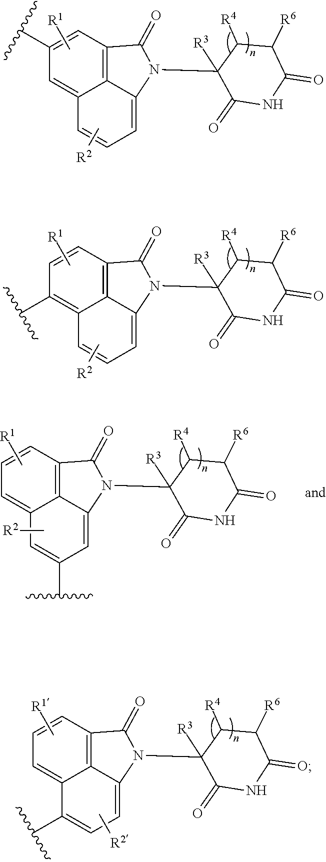

R^{1′} and R^{2′} are independently at each instance selected from hydrogen, alkyl, halogen, haloalkyl, —OR¹⁰, —SR¹⁰, —S(O)R¹², —SO₂R¹², —NR¹⁰R¹¹, cyano, nitro, heteroaryl, aryl, and heterocycle wherein if R^{1′} is hydrogen then R^{2′} is not hydrogen and if R^{2′} is hydrogen than R^{1′} is not hydrogen;
and all other variables are as defined in Formula I or the embodiments described herein.

In certain embodiments, the Tricyclic Cereblon Ligand, with attaching bonds as indicated above, is selected from:

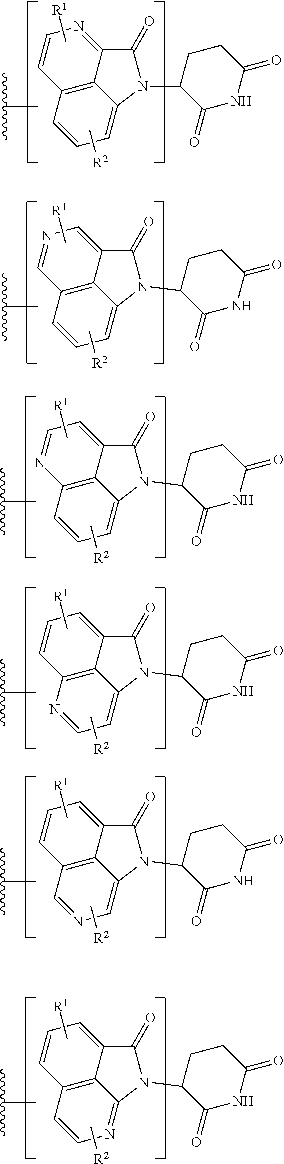

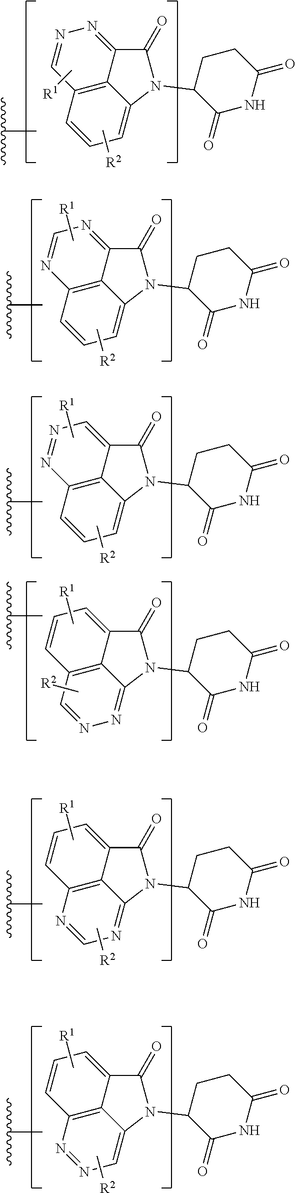

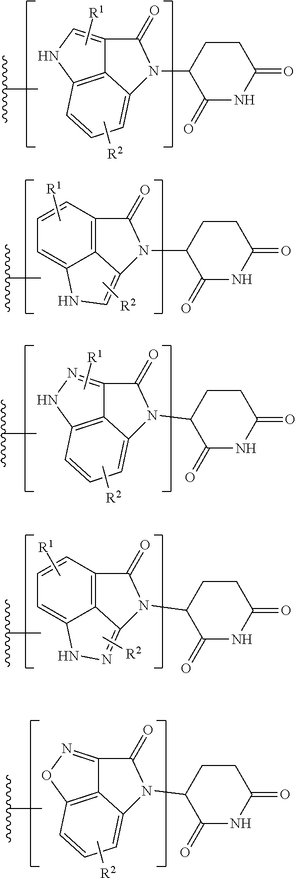

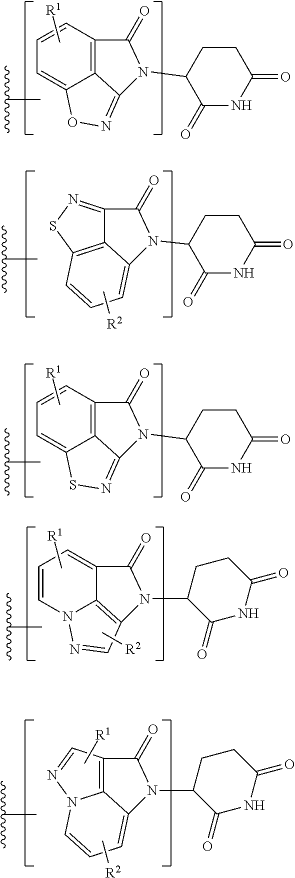

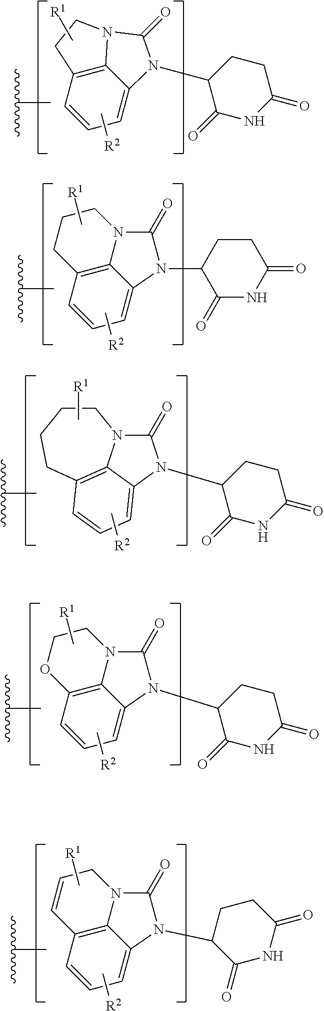

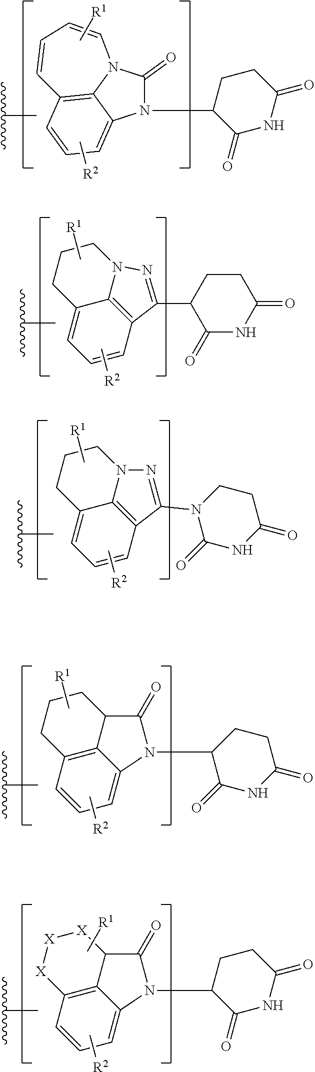

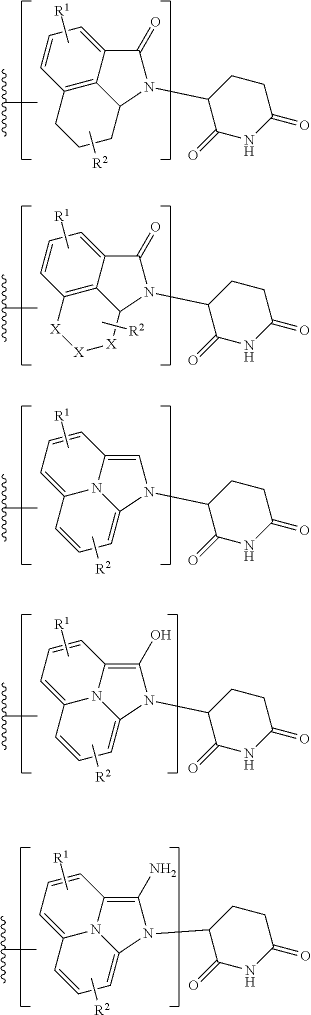

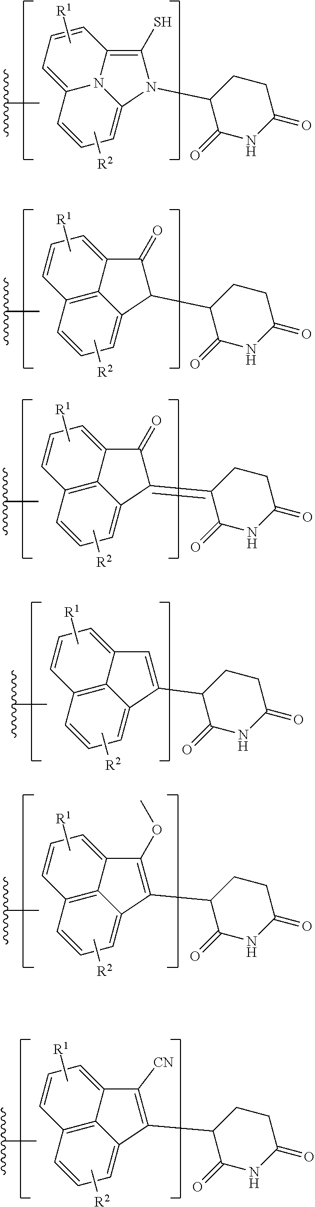

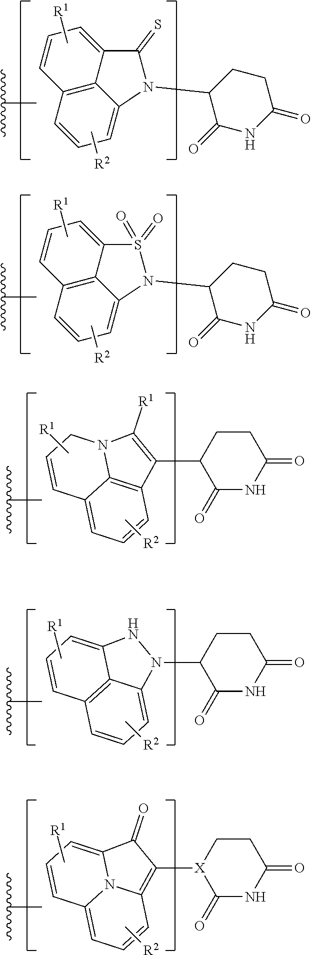

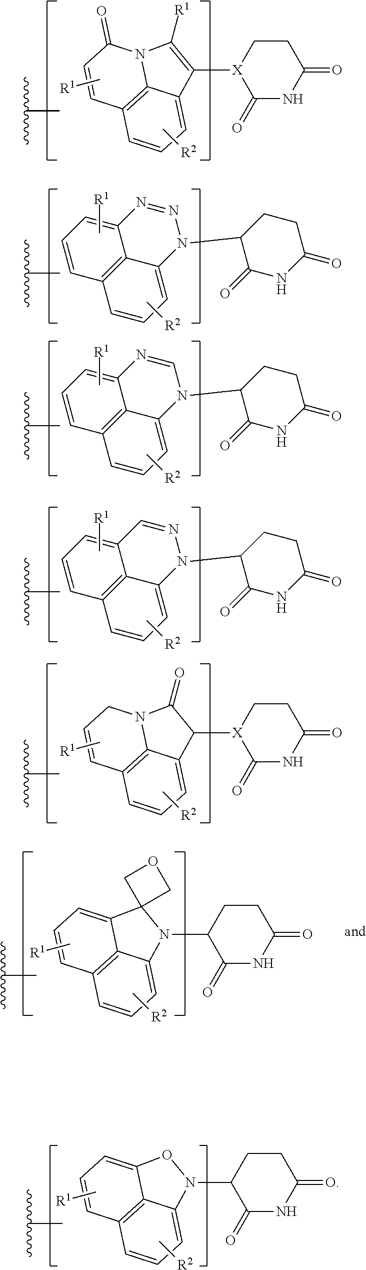

In certain other embodiments, the tricyclic cereblon binding moiety, with attaching bonds as indicated above, is selected from:

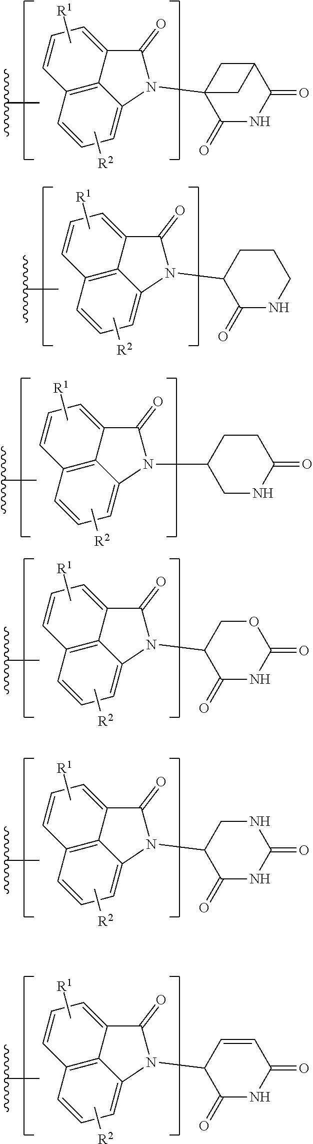

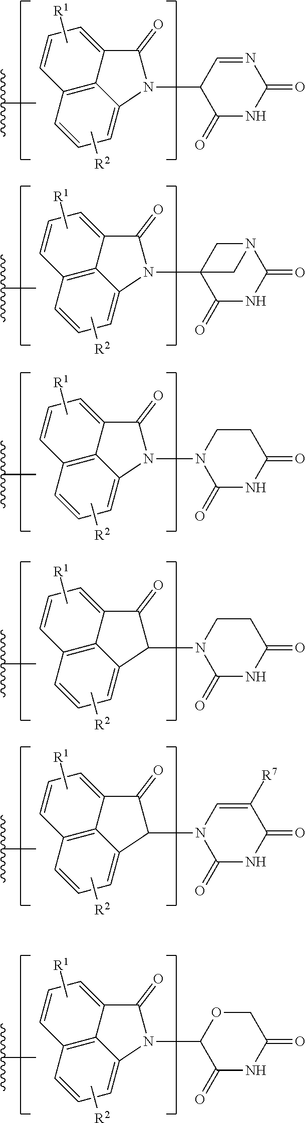

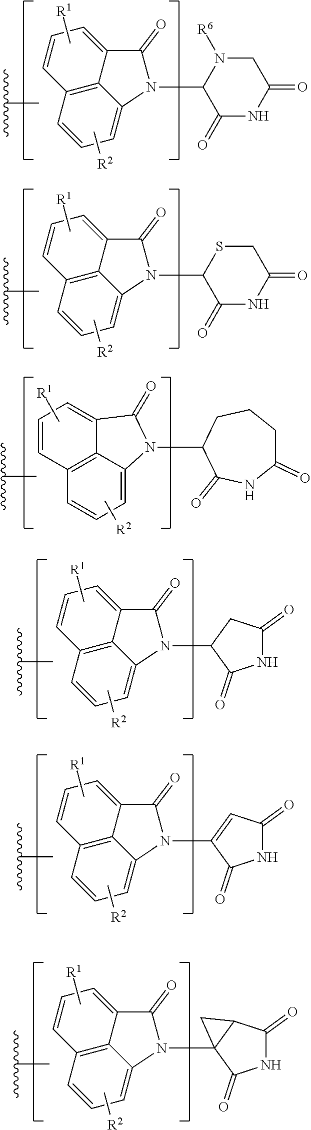

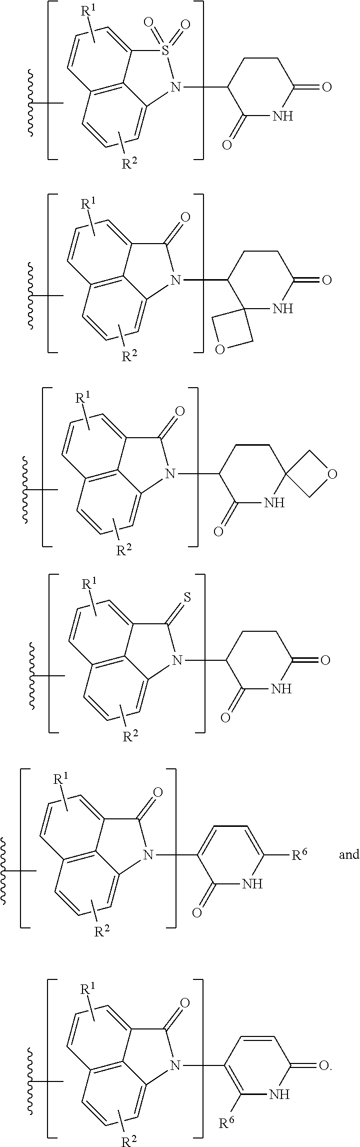

Every combination of variables, substituents, embodiments and the compounds that result from these combinations, is deemed specifically and individually disclosed, as such depiction is for convenience of space only and not intended to describe only a genus or even a subgenus of compounds.

In certain embodiments the compound of the present invention is selected from Formula IIa-1 and IIb-1:

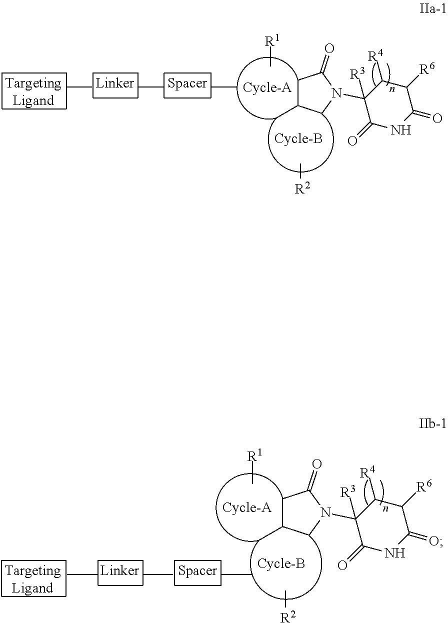

or a pharmaceutically acceptable salt thereof.

In certain embodiments the compound of the present invention is selected from Formula IIa-2 and IIb-2:

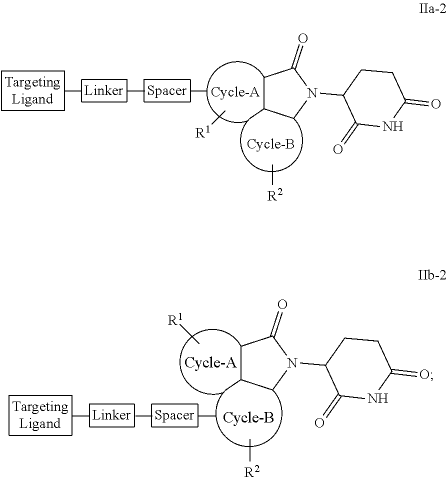

or a pharmaceutically acceptable salt thereof.

In certain embodiments the compound of the present invention is selected from Formula IIa-3 and IIb-3:

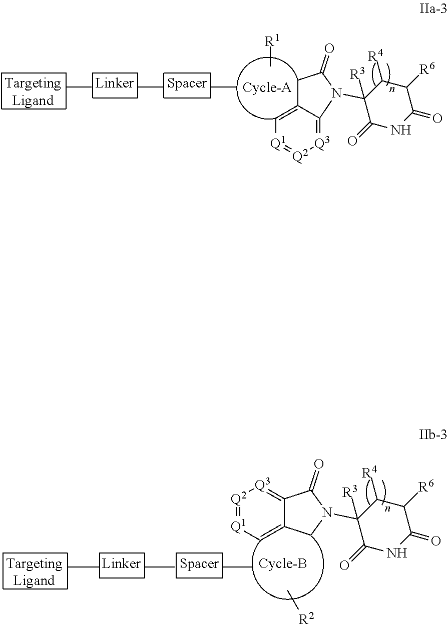

or a pharmaceutically acceptable salt thereof;
wherein:
Q¹, Q², and Q³ are independently selected from CH, CR¹, and N; and all other variables are as defined herein.

In certain embodiments the compound of the present invention is selected from Formula IIa-4 and IIb-4:

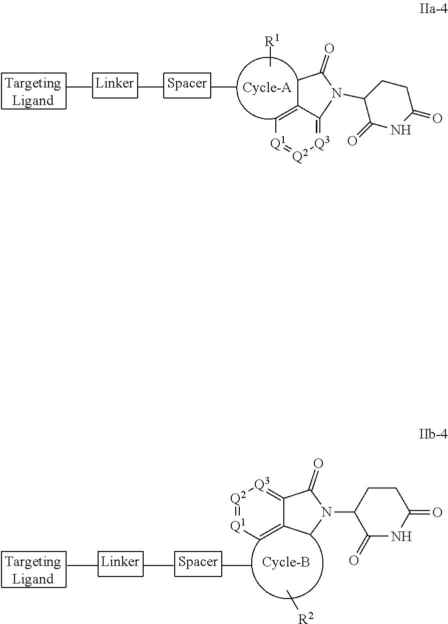

or a pharmaceutically acceptable salt thereof.

In certain embodiments the compound of the present invention is selected from Formula IIa-5 and IIb-5:

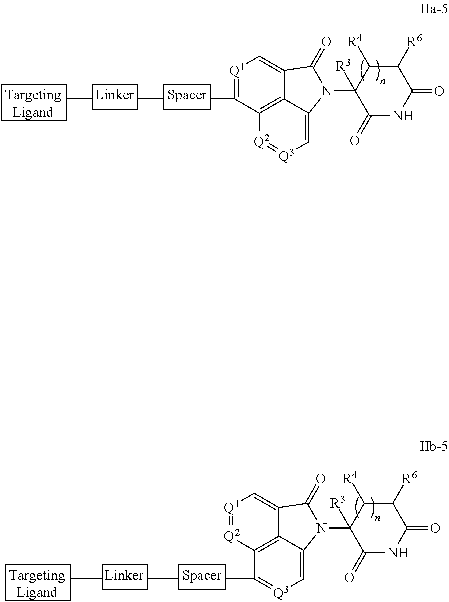

or a pharmaceutically acceptable salt thereof.

In certain embodiments the compound of the present invention is selected from Formula IIa-6 and IIb-6:

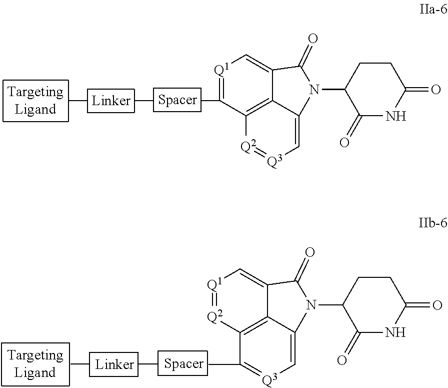

or a pharmaceutically acceptable salt thereof.

In certain embodiments the compound of the present invention is selected from Formula IIa-7 and IIb-7:

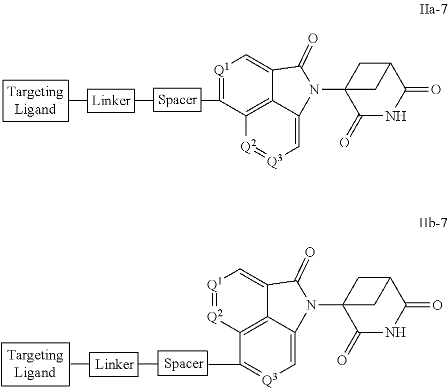

or a pharmaceutically acceptable salt thereof.

Non-limiting examples of compounds of the present invention include:

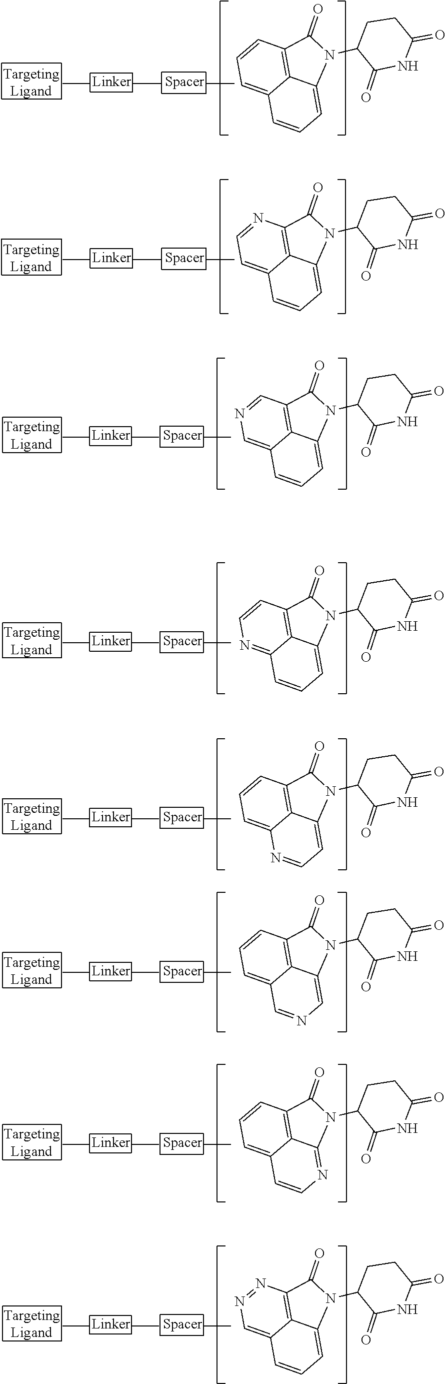

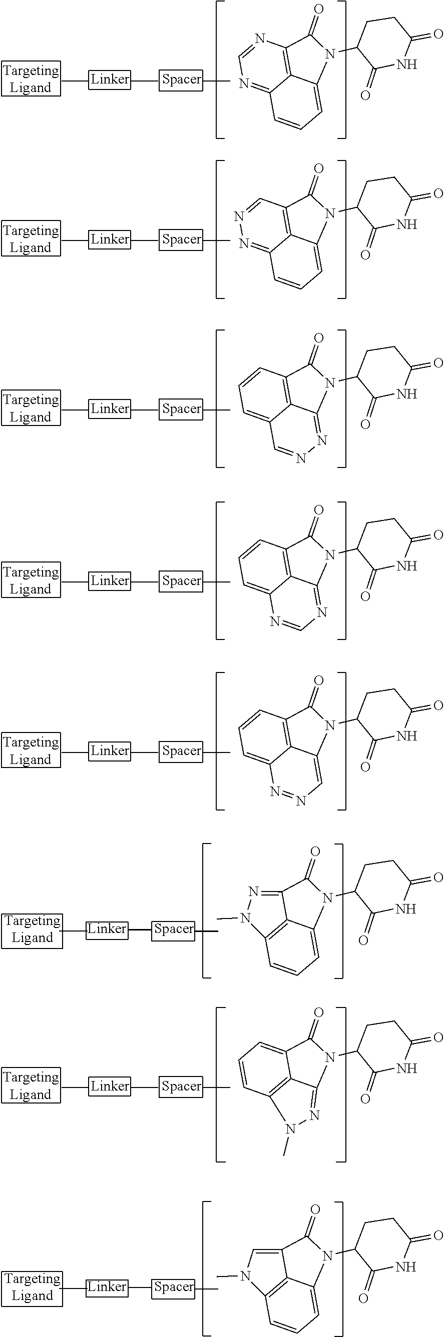

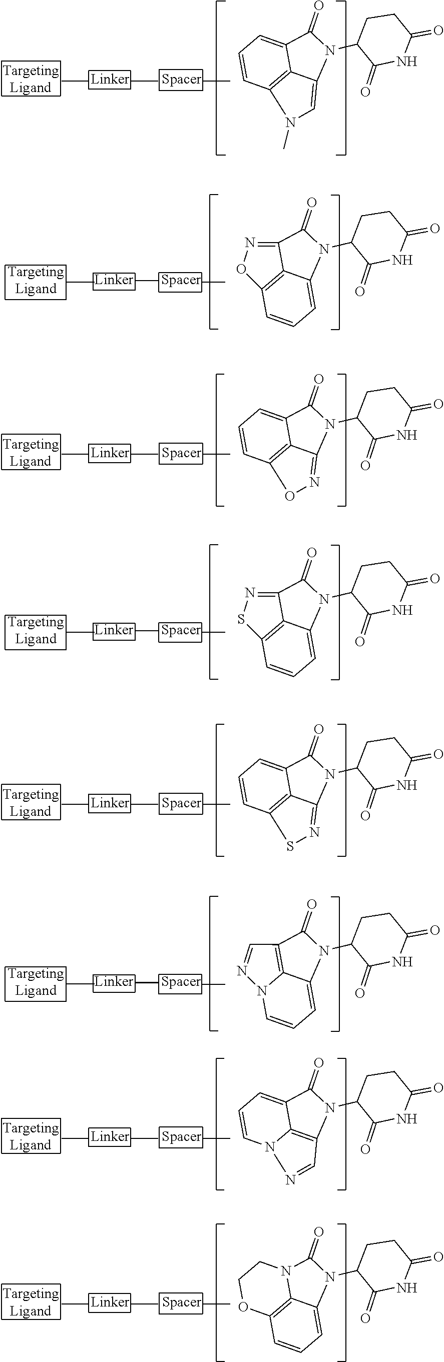

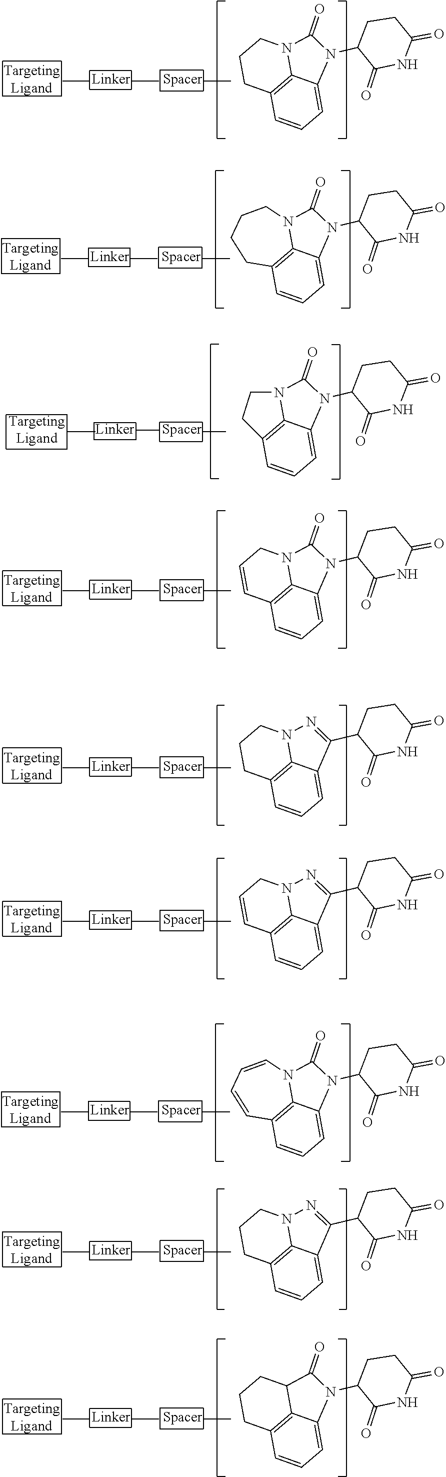

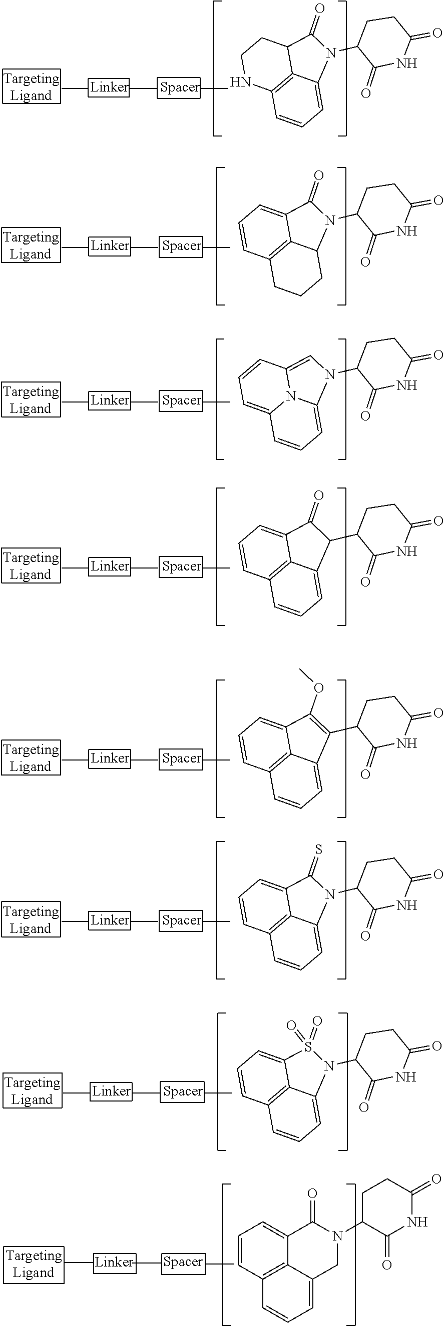

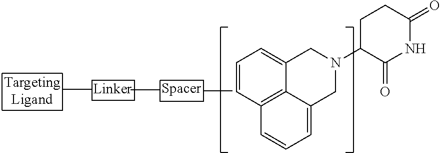

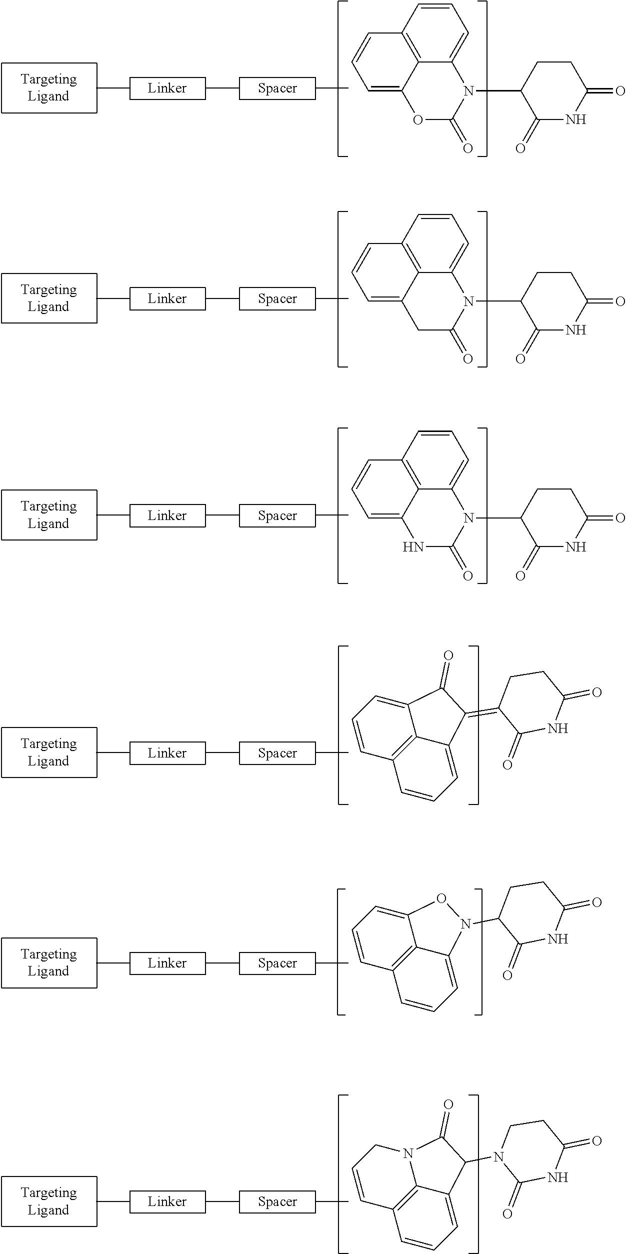

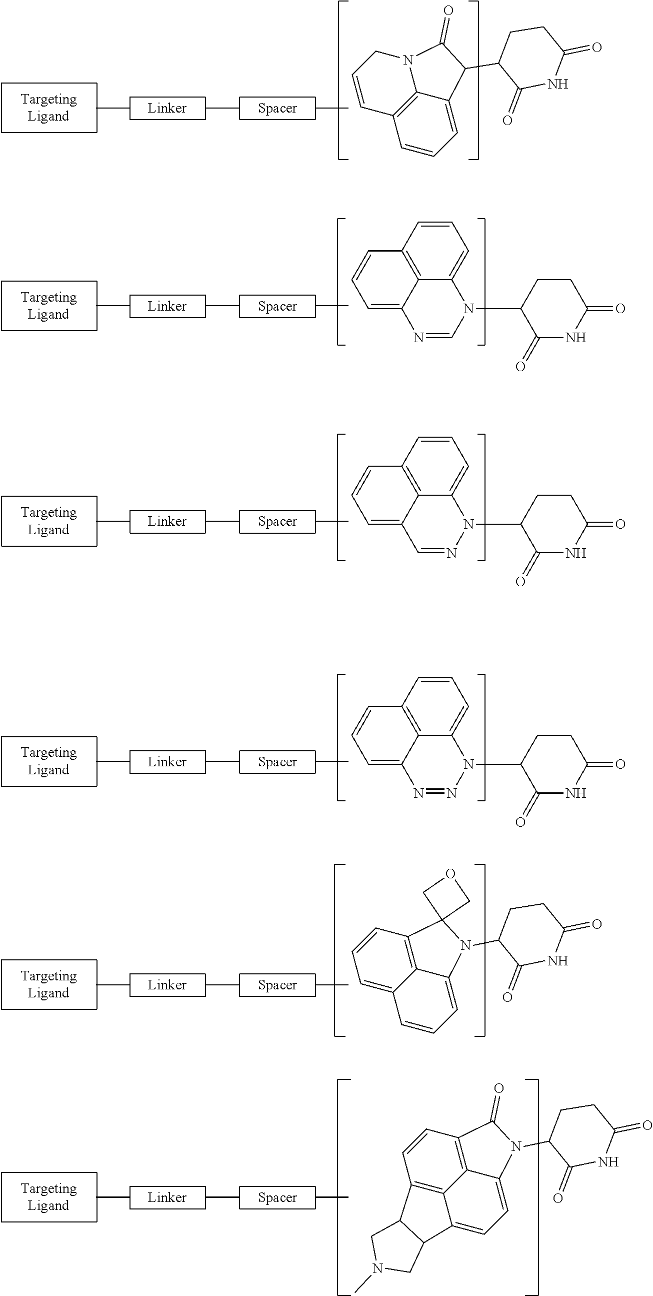

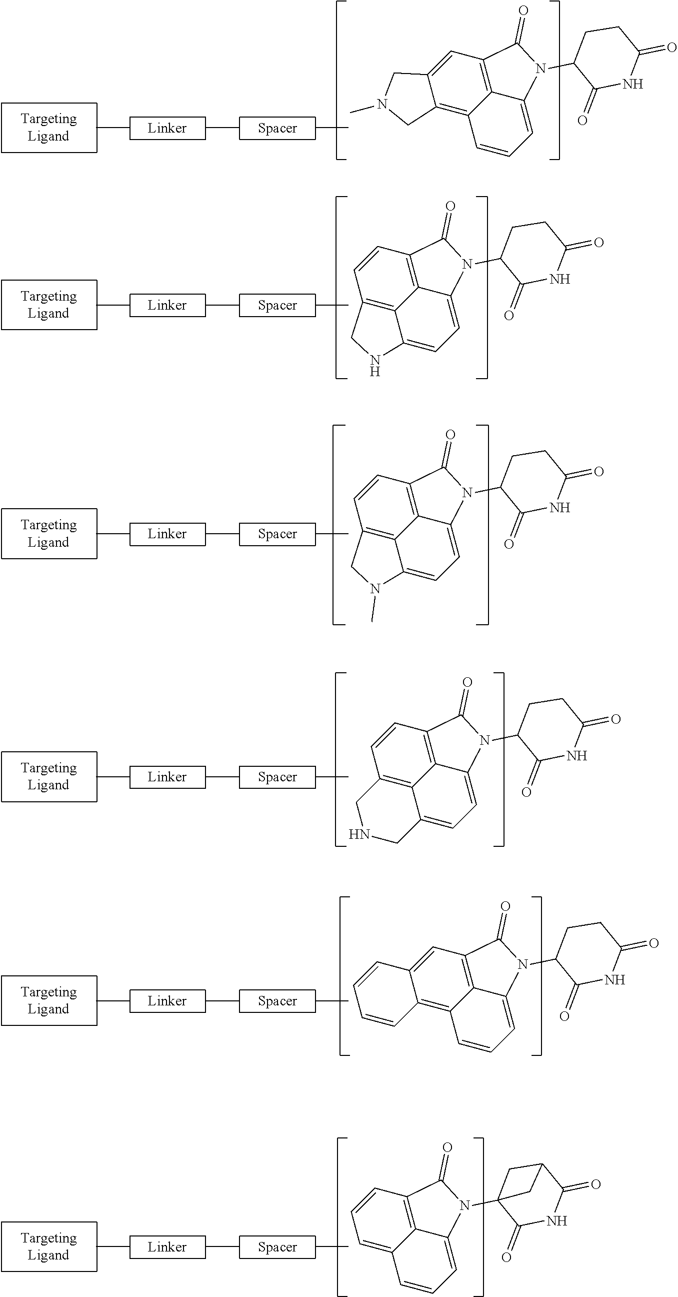

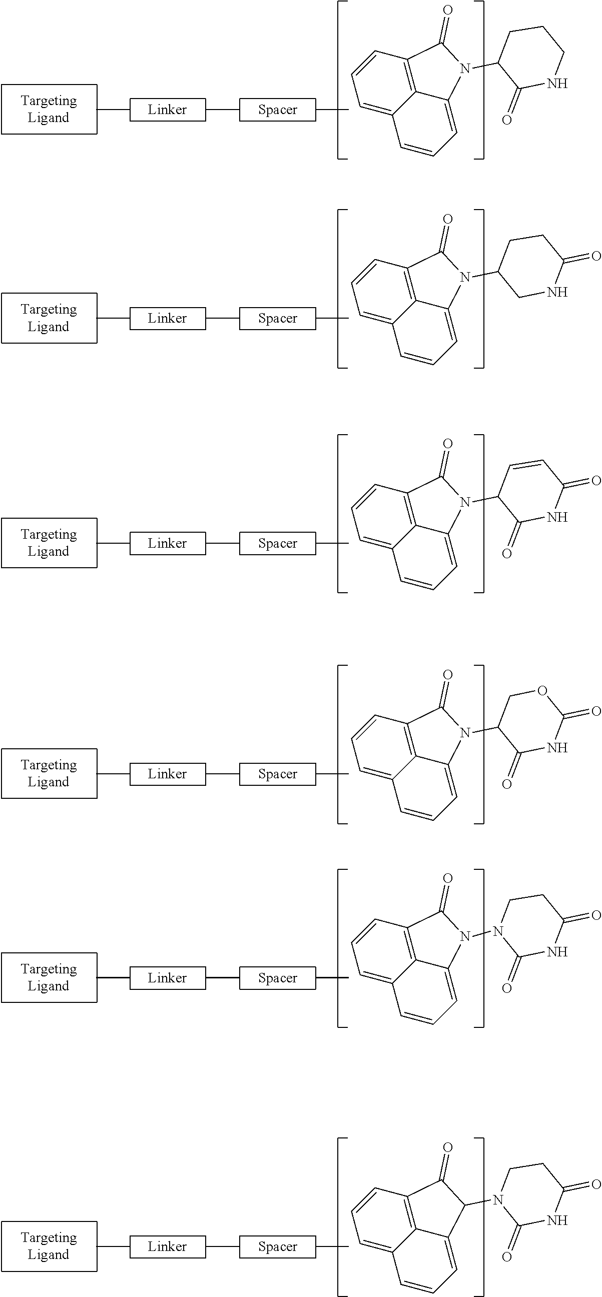

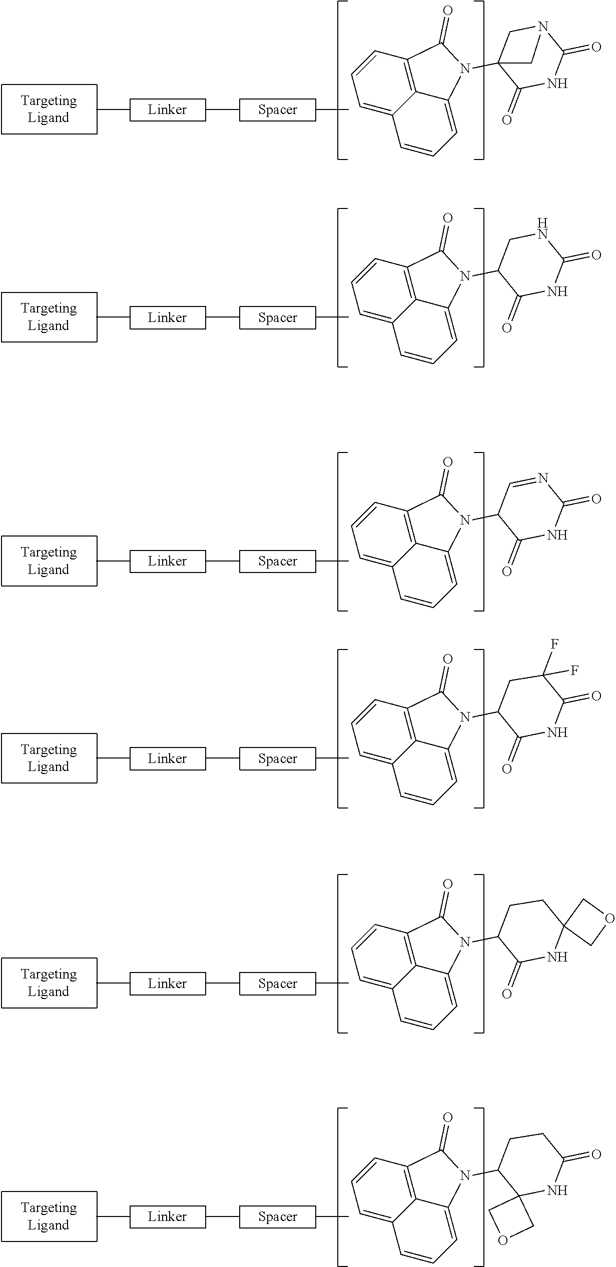

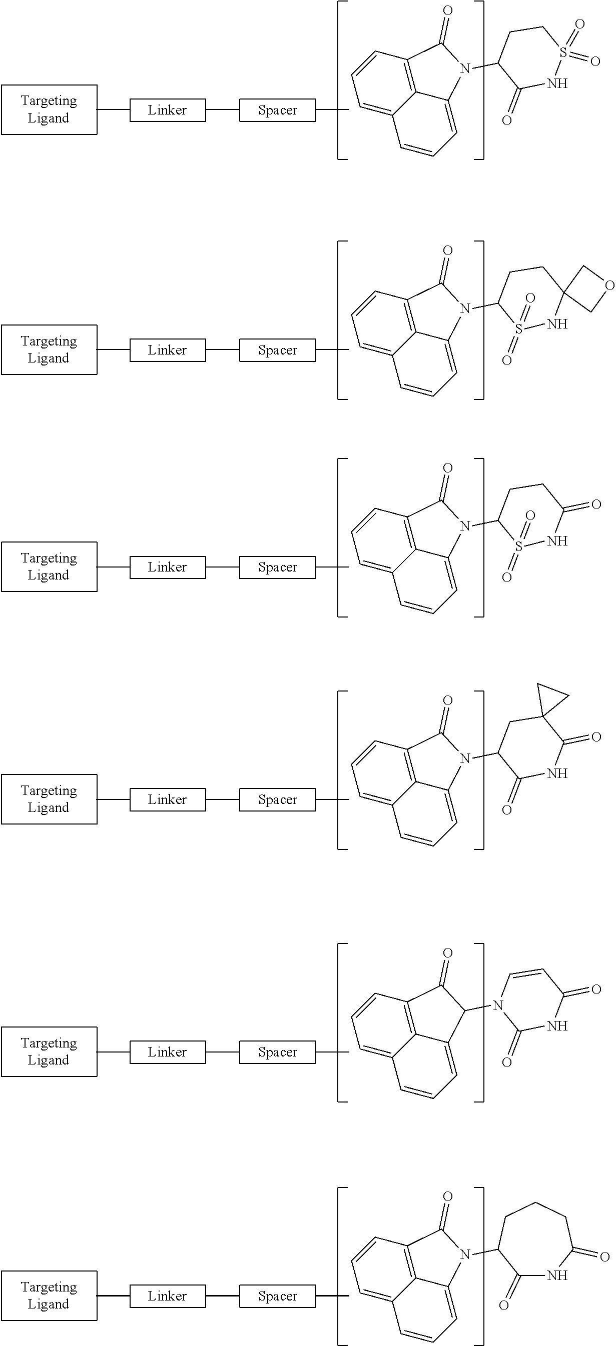

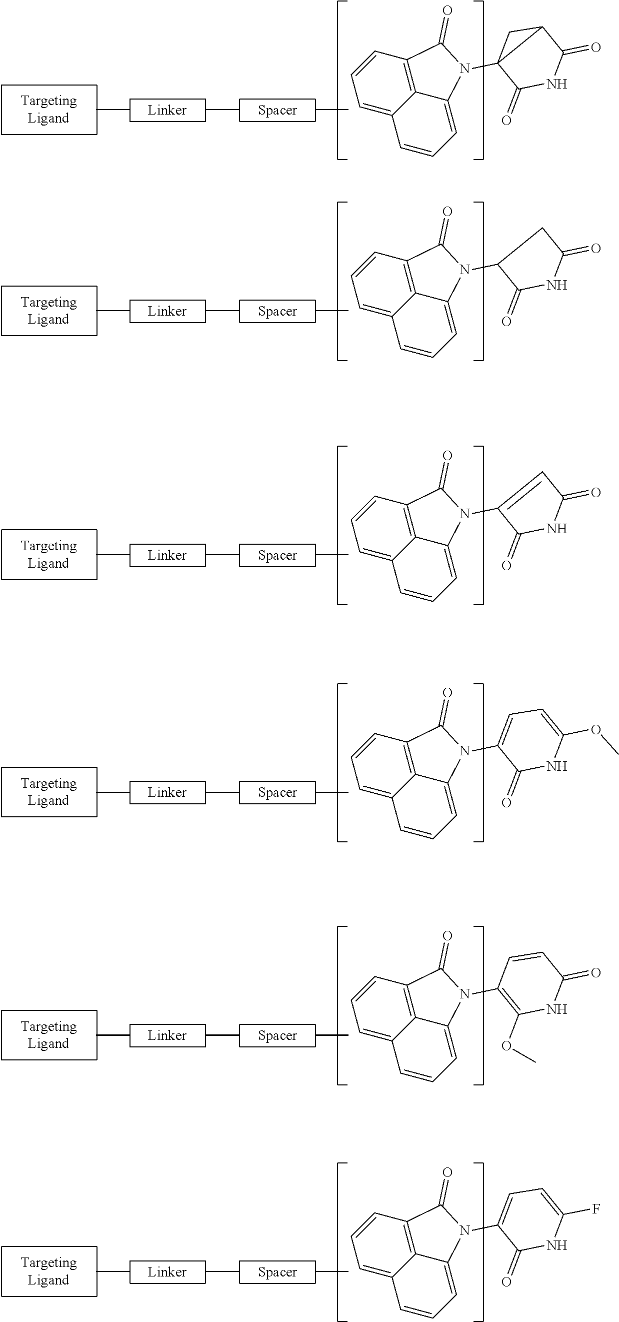

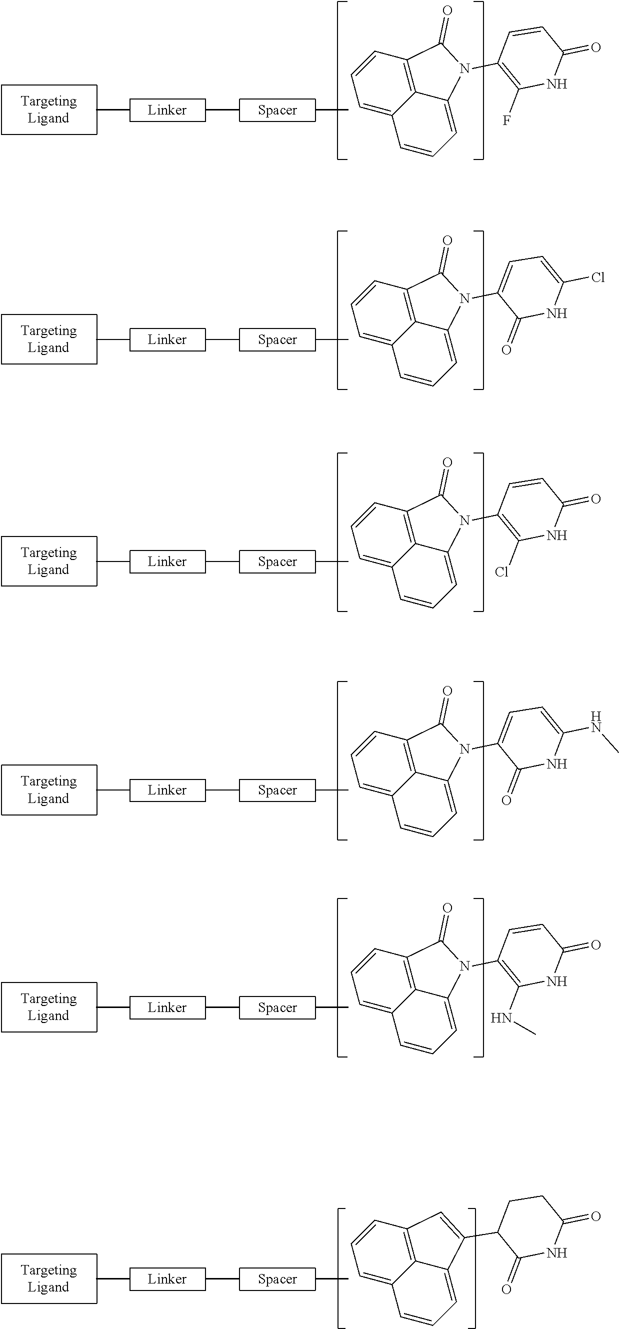

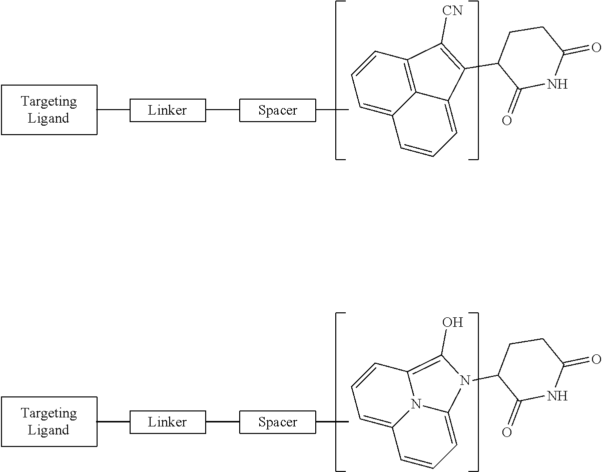

In certain embodiments, a method of treatment is provided comprising administering an effective amount of a compound of Formula I, Formula II, or Formula III or a pharmaceutically acceptable salt thereof to a patient in need thereof, for example a human, optionally in a pharmaceutically acceptable carrier. For example, in certain embodiments, a compound of Formula I, Formula II, or Formula III is administered to a human to treat abnormal cellular proliferation or cancer.

In certain embodiments a compound of the present invention is used to degrade a Target Protein that has an allosteric ligand as the Targeting Ligand. In certain embodiments a compound of the present invention is used to degrade a Target Protein that has an orthosteric ligand as the Targeting Ligand. In certain embodiments a compound of the present invention is used to degrade a Target Protein that is not recruited to the E3 ubiquitin ligase complex via a Targeting Ligand.

In certain embodiments, the compound of the present invention provides one or more, and often multiple advantages over traditional protein inhibition therapy. For example, the tricyclic cereblon heterobifunctional protein degrading compounds of the present invention may a) overcome traditional drug resistance; b) prolong the kinetics of the Target Ligand effect by destroying the protein, thus requiring resynthesis of the protein even after the compound has been metabolized; c) target all functions of the Target Protein at once rather than a specific activity or binding event; d) have increased potency compared to inhibitors due to their catalytic activity; and/or e) require lower dosages than traditional protein inhibitors, decreasing the potential for toxicity.

In certain embodiments, a compound of the present invention is used to treat cancer with a Target Protein that has mutated. In certain embodiments, the Targeting Ligand selectively binds to a mutated protein without significant binding of the wild type protein.

In certain embodiments, a compound of the present invention is used to treat a cancer that is resistant to treatment with the Targeting Ligand alone.

In certain embodiments, the compound of the present invention provides an improved efficacy and/or safety profile relative to the Targeting Ligand alone.

In certain embodiments, a lower concentration of the tricyclic cereblon heterobifunctional protein described herein is needed for treatment of a disorder mediated by the Target Protein, than by the Targeting Ligand alone.

In certain embodiments, an effective amount of the compound of the present invention has less of at least one side-effect in the treatment of a disorder mediated by the Target Protein, than the effective amount of the Targeting Ligand alone.

In certain embodiments, a less frequent dosage of a selected compounds described herein is needed for the effective treatment of a disorder mediated by the Target Protein, than an effective treatment of the Targeting Ligand alone.

Another aspect of the present invention provides a compound as described herein, or an enantiomer, diastereomer, or stereoisomer thereof, or pharmaceutically acceptable salt, hydrate, or solvate thereof, or a pharmaceutical composition, for use in the manufacture of a medicament for inhibiting or preventing a disorder mediated by the Target Protein or for modulating or decreasing the amount of the Target Protein.

Another aspect of the present invention provides a compound as described herein, or an enantiomer, diastereomer, or stereoisomer thereof, or pharmaceutically acceptable salt, hydrate, or solvate thereof, or its pharmaceutical composition, for use in the manufacture of a medicament for treating or preventing a disease mediated by the Target Protein.

In certain embodiments, a selected compound as described herein is useful to treat a disorder comprising an abnormal cellular proliferation, such as a tumor or cancer, wherein the Target Protein is an oncogenic protein or a signaling mediator of the abnormal cellular proliferative pathway and its degradation decreases abnormal cell growth.

In certain embodiments, the selected compound of Formula I, Formula II, or Formula III or its pharmaceutically acceptable salt thereof, has at least one desired isotopic substitution of an atom, at an amount above the natural abundance of the isotope, i.e., enriched.

In certain embodiments, the compound of Formula I, Formula II, or Formula III or its pharmaceutically acceptable salt thereof, includes a deuterium atom or multiple deuterium atoms.

Other features and advantages of the present application will be apparent from the following detailed description.

The present invention thus includes at least the following features:
(a) A compound of Formula I, Formula II, or Formula III as described herein, or a pharmaceutically acceptable salt or isotopic derivative (including a deuterated derivative) thereof or a pharmaceutically acceptable composition thereof;
(b) A method for treating a disorder mediated by a Target Protein, such as an abnormal cellular proliferation, including cancer, comprising administering an effective amount of a compound of Formula I, Formula II, or Formula III, or pharmaceutically acceptable salt thereof, as described herein, to a patient such as a human in need thereof, optionally in a pharmaceutically acceptable composition;
(c) A compound of Formula I, Formula II, or Formula III or a pharmaceutically acceptable salt, or isotopic derivative (including a deuterated derivative) thereof for use in the treatment of a disorder mediated by a Target Protein, for example an abnormal cellular proliferation such as a tumor or cancer, an inflammatory disease, autoimmune disease or fibrotic disease.
(d) Use of a compound of Formula I, Formula II, or Formula III or a pharmaceutically acceptable salt thereof, in an effective amount in the treatment of a patient in need thereof, typically a human, with a disorder mediated by a Target Protein, for example an abnormal cellular proliferation such as a tumor or cancer;
(e) Use of a compound of Formula I, Formula II, or Formula III or a pharmaceutically acceptable salt or isotopic derivative (including a deuterated derivative) thereof in the manufacture of a medicament for the treatment of a disorder mediated by a Target Protein, for example an abnormal cellular proliferation such as a tumor or cancer;
(f) A pharmaceutical composition comprising an effective patient-treating amount of a compound of Formula I, Formula II, or Formula III or a pharmaceutically acceptable salt, isotopic derivative thereof; and optionally a pharmaceutically acceptable carrier or diluent;
(g) A compound Formula I, Formula II, or Formula III as described herein as a mixture of enantiomers or diastereomers (as relevant), including as a racemate;
(h) A compound of Formula I, Formula II, or Formula III as described herein in enantiomerically or diastereomerically (as relevant) enriched form, including an isolated enantiomer or diastereomer (i.e., about greater than 85, 90, 95, 97, or 99% pure); and
(i) A process for the preparation of therapeutic products that contain an effective amount of a compound of Formula I, Formula II, or Formula III or a pharmaceutically acceptable salt thereof, as described herein.

### BRIEF DESCRIPTION OF THE FIGURES

FIG. 1A-1C provide non-limiting examples of Retinoid X Receptor (RXR) Targeting Ligands wherein R represents exemplary points at which the spacer is attached.

FIG. 1D-1F provide non-limiting examples of general Dihydrofolate reductase (DHFR) Targeting Ligands wherein R represents exemplary points at which the spacer is attached.

FIG. 1G provides non-limiting examples of Bacillus anthracis Dihydrofolate reductase (BaDHFR) Targeting Ligands wherein R represents exemplary points at which the spacer is attached.

FIG. 1H-1J provide non-limiting examples of Heat Shock Protein 90 (HSP90) Targeting Ligands wherein R represents exemplary points at which the spacer is attached.

FIG. 1K-1Q provide non-limiting examples of General Kinase and Phosphatase Targeting Ligands wherein R represents exemplary points at which the spacer is attached.

FIG. 1R-1S provides non-limiting examples of Tyrosine Kinase Targeting Ligands wherein R represents exemplary points at which the spacer is attached.

FIG. 1T provides non-limiting examples of Aurora Kinase Targeting Ligands wherein R represents exemplary points at which the spacer is attached.

FIG. 1U provides non-limiting examples of Protein Tyrosine Phosphatase Targeting Ligands wherein R represents exemplary points at which the spacer is attached.

FIG. 1V provides non-limiting examples of ALK Targeting Ligands wherein R represents exemplary points at which the spacer is attached.

FIG. 1W provides non-limiting examples of ABL Targeting Ligands wherein R represents exemplary points at which the spacer is attached.

FIG. 1X provides non-limiting examples of JAK2 Targeting Ligands wherein R represents exemplary points at which the spacer is attached.

FIG. 1Y-1Z provide non-limiting examples of MET Targeting Ligands wherein R represents exemplary points at which the spacer is attached.

FIG. 1AA provides non-limiting examples of mTORC1 and/or mTORC2 Targeting Ligands wherein R represents exemplary points at which the spacer is attached.

FIG. 1BB-1CC provide non-limiting examples of Mast/stem cell growth factor receptor (SCFR), also known as c-KIT receptor, Targeting Ligands wherein R represents exemplary points at which the spacer is attached.

FIG. 1DD provides non-limiting examples of IGF1R and/or IR Targeting Ligands wherein R represents exemplary points at which the spacer is attached.

FIG. 1EE-1FF provide non-limiting examples of HDM2 and/or MDM2 Targeting Ligands wherein R represents exemplary points at which the spacer is attached.

FIG. 1GG-1MM provide non-limiting examples of BET Bromodomain-Containing Protein Targeting Ligands wherein R represents exemplary points at which the spacer is attached.

FIG. 1NN provides non-limiting examples of HDAC Targeting Ligands wherein R represents exemplary points at which the spacer is attached.

FIG. 1OO provides non-limiting examples of RAF Receptor Targeting Ligands wherein R represents exemplary points at which the spacer is attached.

FIG. 1PP provides non-limiting examples of FKBP Receptor Targeting Ligands wherein R represents exemplary points at which the spacer is attached.

FIG. 1QQ-1TT provide non-limiting examples of Androgen Receptor Targeting Ligands wherein R represents exemplary points at which the spacer is attached.

FIG. 1UU provides non-limiting examples of Estrogen Receptor Targeting Ligands wherein R represents exemplary points at which the spacer is attached.

FIG. 1VV-1WW provide non-limiting examples of Thyroid Hormone Receptor Targeting Ligands wherein R represents exemplary points at which the spacer is attached.

FIG. 1XX provides non-limiting examples of HIV Protease Targeting Ligands wherein R represents exemplary points at which the spacer is attached.

FIG. 1YY provides non-limiting examples of HIV Integrase Targeting Ligands wherein R represents exemplary points at which the spacer is attached.

FIG. 1ZZ provides non-limiting examples of HCV Protease Targeting Ligands wherein R represents exemplary points at which the spacer is attached.

FIG. 1AAA provides non-limited examples of AP1 and/or AP2 Targeting Ligands wherein R represents exemplary points at which the spacer is attached.

FIG. 1BBB-1CCC provide non-limiting examples of MCL-1 Targeting Ligands wherein R represents exemplary points at which the spacer is attached.

FIG. 1DDD provides non-limiting examples of IDH1 Targeting Ligands wherein R represents exemplary points at which the spacer is attached.

FIG. 1EEE-1FFF provide non-limiting examples of RAS or RASK Targeting Ligands wherein R represents exemplary points at which the spacer is attached.

FIG. 1GGG provides non-limiting examples of MERTK or MER Targeting Ligands wherein R represents exemplary points at which the spacer is attached.

FIG. 1HHH-1III provide non-limiting examples of EGFR Targeting Ligands wherein R represents exemplary points at which the spacer is attached.

FIG. 1JJJ-1KKK provide non-limiting examples of FLT3 Targeting Ligands wherein R represents exemplary points at which the spacer is attached.

FIG. 1LLL provides non-limiting examples of SMARCA2 Targeting Ligands wherein R represents exemplary points at which the spacer is attached.

FIG. 2A provides non-limiting examples of the kinase inhibitor Targeting Ligands U09-CX-5279 (derivatized) wherein R represents exemplary points at which the spacer is attached.

FIG. 2B-2C provide non-limiting examples of kinase inhibitor Targeting Ligands, including the kinase inhibitor compounds Y1W and Y1X (derivatized) wherein R represents exemplary points at which the spacer is attached. For additional examples and related ligands, see, the kinase inhibitors identified in Millan et al. “Design and Synthesis of Inhaled P38 Inhibitors for the Treatment of Chronic Obstructive Pulmonary Disease” J. Med. Chem., 54: 7797 (2011).

FIG. 2D provides non-limiting examples of kinase inhibitor Targeting Ligands, including the kinase inhibitor compounds 6TP and OTP (derivatized) wherein R represents exemplary points at which the spacer is attached. For additional examples and related ligands, see, the kinase inhibitors identified in Schenkel et al. “Discovery of Potent and Highly Selective Thienopyridine Janus Kinase 2 Inhibitors” J. Med. Chem., 54 (24): 8440-8450 (2011).

FIG. 2E provides non-limiting examples of kinase inhibitor Targeting Ligands, including the kinase inhibitor compound 07U wherein R represents exemplary points at which the spacer is attached. For additional examples and related ligands, see, the kinase inhibitors identified in Van Eis et al. “2 6-Naphthyridines as potent and selective inhibitors of the novel protein kinase C isozymes” Biorg. Med. Chem. Lett., 21(24): 7367-72 (2011).

FIG. 2F provides non-limiting examples of kinase inhibitor Targeting Ligands, including the kinase inhibitor compound YCF, wherein R represents exemplary points at which the spacer is attached. For additional examples and related ligands, see, the kinase inhibitors identified in Lountos et al. “Structural Characterization of Inhibitor Complexes with Checkpoint Kinase 2 (Chk2) a Drug Target for Cancer Therapy” J. Struct. Biol., 176: 292 (2011).

FIG. 2G-2H provide non-limiting examples of kinase inhibitor Targeting Ligands, including the kinase inhibitors XK9 and NXP (derivatized) wherein R represents exemplary points at which the spacer is attached. For additional examples and related ligands, see, the kinase inhibitors identified in Lountos et al. “Structural Characterization of Inhibitor Complexes with Checkpoint Kinase 2 (Chk2) a Drug Target for Cancer Therapy” J. Struct. Biol., 176: 292 (2011).

FIG. 2I-2J provide non-limiting examples of kinase inhibitor Targeting Ligands wherein R represents exemplary points at which the spacer r is attached.

FIG. 2K-2M provide non-limiting examples of Cyclin Dependent Kinase 9 (CDK9) Targeting Ligands wherein R represents exemplary points at which the spacer is attached. For additional examples and related ligands, see, Baumli et al. “The structure of P-TEFb (CDK9/cyclin T1) its complex with flavopiridol and regulation by phosphorylation.” Embo J., 27: 1907-1918 (2008); Bettayeb et al. “CDK Inhibitors Roscovitine and CR8 Trigger Mcl-1 Down-Regulation and Apoptotic Cell Death in Neuroblastoma Cells.” Genes Cancer, 1: 369-380 (2010); Baumli et al. “Halogen bonds form the basis for selective P-TEFb inhibition by DRB.” Chem. Biol. 17: 931-936 (2010); Hole et al. “Comparative Structural and Functional Studies of 4-(Thiazol-5-Yl)-2-(Phenylamino)Pyrimidine-5-Carbonitrile Cdk9 Inhibitors Suggest the Basis for Isotype Selectivity.” J. Med. Chem. 56: 660 (2013); Lücking et al. “Identification of the potent and highly selective PTEFb inhibitor BAY 1251152 for the treatment of cancer—From p.o. to i.v. application via scaffold hops.” Lücking et al. U. AACR Annual Meeting, Apr. 1-5, 2017 Washington, D.C. USA.

FIG. 2N-2P provide non-limiting examples of Cyclin Dependent Kinase 4/6 (CDK4/6) Targeting Ligands wherein R represents exemplary points at which the spacer is attached. For additional examples and related ligands, see, Lu H.; Schulze-Gahmen U.; “Toward understanding the structural basis of cyclin-dependent kinase 6 specific inhibition.” J. Med. Chem., 49: 3826-3831 (2006); 4-(Pyrazol-4-yl)-pyrimidines as selective inhibitors of cyclin-dependent kinase 4/6. Cho et al. (2010) J. Med. Chem. 53: 7938-7957; Cho Y. S. et al. “Fragment-Based Discovery of 7-Azabenzimidazoles as Potent Highly Selective and Orally Active CDK4/6 Inhibitors.” ACS Med Chem Lett 3: 445-449 (2012); Li Z. et al. “Discovery of AMG 925 a FLT3 and CDK4 dual kinase inhibitor with preferential affinity for the activated state of FLT3.” J. Med. Chem. 57: 3430-3449 (2014); Chen P. et al. “Spectrum and Degree of CDK Drug Interactions Predicts Clinical Performance.” Mol. Cancer Ther. 15: 2273-2281 (2016).

FIG. 2Q provides non-limiting examples of Cyclin Dependent Kinase 12 and/or Cyclin Dependent Kinase 13 Targeting Ligands wherein R represents exemplary points at which the spacer is attached. For additional examples and related ligands, see, Zhang T. et al. “Covalent Targeting of Remote Cysteine Residues to Develop Cdk12 and Cdk13 Inhibitors.” Nat. Chem. Biol. 12: 876 (2016).

FIG. 2R-2S provide non-limiting examples of Glucocorticoid Receptor Targeting Ligands wherein R represents exemplary points at which the spacer is attached.

FIG. 2T-2U provide non-limiting examples of RasG12C Targeting Ligands wherein R represents exemplary points at which the spacer is attached.

FIG. 2V provides non-limiting examples of Her3 Targeting Ligands wherein R represents exemplary points at which the spacer is attached and R″ is

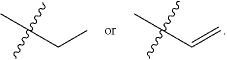

FIG. 2W provides non-limiting examples of Bel-2 or Bcl-XL Targeting Ligands wherein R represents exemplary points at which the spacer is attached.

FIG. 2X-2NN provide non-limiting examples of BCL2 Targeting Ligands wherein R represents exemplary points at which the spacer is attached. For additional examples and related ligands, see, Toure B. B. et al. “The role of the acidity of N-heteroaryl sulfonamides as inhibitors of bcl-2 family protein-protein interactions.” ACS Med Chem Lett, 4: 186-190 (2013); Porter J. et al. “Tetrahydroisoquinoline Amide Substituted Phenyl Pyrazoles as Selective Bcl-2 Inhibitors” Bioorg. Med. Chem. Lett. 19: 230 (2009); Souers A. J. et al. “ABT-199 a potent and selective BCL-2 inhibitor achieves antitumor activity while sparing platelets.” Nature Med. 19: 202-208 (2013); Angelo Aguilar et al. “A Potent and Highly Efficacious Bcl-2/Bcl-xL Inhibitor” J. Med. Chem. 56(7): 3048-3067 (2013); Longchuan Bai et al. “BM-1197: A Novel and Specific Bcl-2/Bcl-xL Inhibitor Inducing Complete and Long-Lasting Tumor Regression In Vivo” PLoS ONE 9(6): e99404; Fariba Ne'matil et al. “Targeting Bcl-2/Bcl-XL Induces Antitumor Activity in Uveal Melanoma Patient-Derived Xenografts” PLoS ONE 9(1): e80836; WO2015011396 titled “Novel derivatives of indole and pyrrole method for the production thereof and pharmaceutical compositions containing same”; WO2008060569A1 titled “Compounds and methods for inhibiting the interaction of Bcl proteins with binding partners”; “Inhibitors of the anti-apoptotic Bcl-2 proteins: a patent review” Expert Opin. Ther. Patents 22(1):2008 (2012); and, Porter et al. “Tetrahydroisoquinoline amide substituted phenyl pyrazoles as selective Bcl-2 inhibitors” Bioorg Med Chem Lett., 19(1):230-3 (2009).

FIG. 2OO-2UU provide non-limiting examples of BCL-XL Targeting Ligands wherein R represents exemplary points at which the spacer is attached. For additional examples and related ligands, see, Zhi-Fu Tao et al. “Discovery of a Potent and Selective BCL-XL Inhibitor with in Vivo Activity” ACS Med. Chem. Lett., 5: 1088-1093 (2014); Joel D. Leverson et al. “Exploiting selective BCL-2 family inhibitors to dissect cell survival dependencies and define improved strategies for cancer therapy” Science Translational Medicine, 7:279ra40 (2015); and, the crystal structure PDB 3ZK6 (Guillaume Lessene et al. “Structure-guided design of a selective BCL-XL inhibitor” Nature Chemical Biology 9: 390-397 (2013))

FIG. 2VV provides non-limiting examples of PPAR-gamma Targeting Ligands wherein R represents exemplary points at which the spacer is attached.

FIG. 2WW-2YY provide non-limiting examples of EGFR Targeting Ligands that target the EGFR L858R mutant, including erlotinib, gefitnib, afatinib, neratinib, and dacomitinib, wherein R represents exemplary points at which the spacer is attached.

FIG. 2ZZ-2FFF provide non-limiting examples of EGFR Targeting Ligands that target the EGFR T790M mutant, including osimertinib, rociletinib, olmutinib, naquotinib, nazartinib, PF-06747775, Icotinib, Neratinib Avitinib, Tarloxotinib, PF-0645998, Tesevatinib, Transtinib, WZ-3146, WZ8040, and CNX-2006, wherein R represents exemplary points at which the spacer is attached.

FIG. 2GGG provides non-limiting examples of EGFR Targeting Ligands that target the EGFR C797S mutant, including EAI045, wherein R represents exemplary points at which the spacer is attached.

FIG. 2HHH provides non-limiting examples of BCR-ABL Targeting Ligands that target the BCR-ABL T315I mutant including Nilotinib and Dasatinib, wherein R represents exemplary points at which the spacer is attached. See for example, the crystal structure PDB 3CS9.

FIG. 2III provides non-limiting examples of Targeting Ligands that target BCR-ABL, including Nilotinib, Dasatinib Ponatinib and Bosutinib, wherein R represents exemplary points at which the spacer is attached.

FIG. 2JJJ-2KKK provide non-limiting examples of ALK Targeting Ligands that target the ALK L1196M mutant including Ceritinib, wherein R represents exemplary points at which the spacer is attached. See for example, the crystal structure PDB 4MKC.

FIG. 2LLL provides non-limiting examples of JAK2 Targeting Ligands that target the JAK2V617F mutant, including Ruxolitinib, wherein R represents exemplary points at which the spacer is attached.

FIG. 2MMM provides non-limiting examples of BRAF Targeting Ligands that target the BRAF V600E mutant including Vemurafenib, wherein R represents exemplary points at which the spacer is attached. For additional examples and related ligands, see, the crystal structure PBD 3OG7.

FIG. 2NNN provides non-limiting examples of BRAF Targeting Ligands, including Dabrafenib, wherein R represents exemplary points at which the spacer is attached.

FIG. 2OOO provides non-limiting examples of LRRK2 Targeting Ligands that target the LRRK2 R1441C mutant wherein R represents exemplary points at which the spacer is attached.

FIG. 2PPP provides non-limiting examples of LRRK2 Targeting Ligands that target the LRRK2 G2019S mutant wherein R represents exemplary points at which the spacer is attached.

FIG. 2QQQ provides non-limiting examples of LRRK2 Targeting Ligands that target the LRRK2 I2020T mutant wherein R represents exemplary points at which the spacer is attached.

FIG. 2RRR-2TTT provide non-limiting examples of PDGFRα Targeting Ligands that target the PDGFRα T674I mutant, including AG-1478, CHEMBL94431, Dovitinib, erlotinib, gefitinib, imatinib, Janex 1, Pazopanib, PD153035, Sorafenib, Sunitinib, and WHI-P180, wherein R represents exemplary points at which the spacer is attached.

FIG. 2UUU provides non-limiting examples of RET Targeting Ligands that target the RET G691S mutant, including tozasertib, wherein R represents exemplary points at which the spacer is attached.

FIG. 2VVV provides non-limiting examples of RET Targeting Ligands that target the RET R749T mutant, including tozasertib, wherein R represents exemplary points at which the spacer is attached.

FIG. 2WWW provides non-limiting examples of RET Targeting Ligands that target the RET E762Q mutant, including tozasertib, wherein R represents exemplary points at which the spacer is attached.

FIG. 2XXX provides non-limiting examples of RET Targeting Ligands that target the RET Y791F mutant, including tozasertib, wherein R represents exemplary points at which the spacer is attached.

FIG. 2YYY provides non-limiting examples of RET Targeting Ligands that target the RET V804M mutant, including tozasertib, wherein R represents exemplary points at which the spacer is attached.

FIG. 2ZZZ provides non-limiting examples of RET Targeting Ligands that target the RET M918T mutant, including tozasertib, wherein R represents exemplary points at which the spacer is attached.

FIG. 2AAAA provides non-limiting examples of Fatty Acid Binding Protein Targeting Ligands wherein R represents exemplary points at which the spacer is attached.

FIG. 2BBBB provides non-limiting examples of 5-Lipoxygenase Activating Protein (FLAP) Targeting Ligands wherein R represents exemplary points at which the spacer is attached.

FIG. 2CCCC provides non-limiting examples of Kringle Domain V 4BVV Targeting Ligands wherein R represents exemplary points at which the spacer is attached.

FIG. 2DDDD provides non-limiting examples of Lactoylglutathione Lyase Targeting Ligands wherein R represents exemplary points at which the spacer is attached.

FIG. 2EEEE-2FFFF provide non-limiting examples of mPGES-1 Targeting Ligands wherein R represents exemplary points at which the spacer is attached.

FIG. 2GGGG-2JJJJ provide non-limiting examples of Factor Xa Targeting Ligands wherein R represents exemplary points at which the spacer is attached. For additional examples and related ligands, see, Maignan S. et al. “Crystal structures of human factor Xa complexed with potent inhibitors.” J. Med. Chem. 43: 3226-3232 (2000); Matsusue T. et al. “Factor Xa Specific Inhibitor that Induces the Novel Binding Model in Complex with Human Fxa.” (to be published); the crystal structures PDB 1iqh, 1iqi, 1iqk, and 1iqm; Adler M. et al. “Crystal Structures of Two Potent Nonamidine Inhibitors Bound to Factor Xa.” Biochemistry 41: 15514-15523 (2002); Roehrig S. et al. “Discovery of the Novel Antithrombotic Agent 5-Chloro-N-({(5S)-2-Oxo-3-[4-(3-Oxomorpholin-4-Yl)Phenyl]-1 3-Oxazolidin-5-Yl}Methyl)Thiophene-2-Carboxamide (Bay 59-7939): An Oral Direct Factor Xa Inhibitor.” J. Med. Chem. 48: 5900 (2005); Anselm L. et al. “Discovery of a Factor Xa Inhibitor (3R 4R)-1-(2 2-Difluoro-Ethyl)-Pyrrolidine-3 4-Dicarboxylic Acid 3-[(5-Chloro-Pyridin-2-Yl)-Amide] 4-{[2-Fluoro-4-(2-Oxo-2H-Pyridin-1-Yl)-Phenyl]-Amide} as a Clinical Candidate.” Bioorg. Med. Chem. 20: 5313 (2010); and, Pinto D. J. et al. “Discovery of 1-(4-Methoxyphenyl)-7-oxo-6-(4-(2-oxopiperidin-1-yl)phenyl)-4 5 6 7-tetrahydro-1H-pyrazolo[3 4-c]pyridine-3-carboxamide (Apixaban BMS-562247) a Highly Potent Selective Efficacious and Orally Bioavailable Inhibitor of Blood Coagulation Factor Xa.” J. Med. Chem. 50: 5339-5356 (2007).

FIG. 2KKKK provides non-limiting examples of Kallikrein 7 Targeting Ligands wherein R represents exemplary points at which the spacer is attached. For additional examples and related ligands, see, Maibaum J. et al. “Small-molecule factor D inhibitors targeting the alternative complement pathway.” Nat. Chem. Biol. 12: 1105-1110 (2016).

FIG. 2LLLL-2MMMM provide non-limiting examples of Cathepsin K Targeting Ligands wherein R represents exemplary points at which the spacer is attached. For additional examples and related ligands, see, Rankovic Z. et al. “Design and optimization of a series of novel 2-cyano-pyrimidines as cathepsin K inhibitors” Bioorg. Med. Chem. Lett. 20: 1524-1527 (2010); and, Cai J. et al. “Trifluoromethylphenyl as P2 for ketoamide-based cathepsin S inhibitors.” Bioorg. Med Chem. Lett. 20: 6890-6894 (2010).

FIG. 2NNNN provides non-limiting examples of Cathepsin L Targeting Ligands wherein R represents exemplary points at which the spacer is attached. For additional examples and related ligands, see, Kuhn B. et al. “Prospective Evaluation of Free Energy Calculations for the Prioritization of Cathepsin L Inhibitors.” J. Med. Chem. 60: 2485-2497 (2017).

FIG. 2OOOO provides non-limiting examples of Cathepsin S Targeting Ligands wherein R represents exemplary points at which the spacer is attached. For additional examples and related ligands, see, Jadhav P. K. et al. “Discovery of Cathepsin S Inhibitor LY3000328 for the Treatment of Abdominal Aortic Aneurysm” ACS Med. Chem. Lett. 5: 1138-1142.” (2014).

FIG. 2PPPP-2SSSS provide non-limiting examples of MTH1 Targeting Ligands wherein R represents exemplary points at which the spacer is attached. For additional examples and related ligands, see, Kettle J. G. et al. “Potent and Selective Inhibitors of Mth1 Probe its Role in Cancer Cell Survival.” J. Med. Chem. 59: 2346 (2016); Huber K. V. M. et al. “Stereospecific Targeting of Mth1 by (S)-Crizotinib as an Anticancer Strategy.” Nature 508: 222 (2014); Gad H. et al. “MTH1 inhibition eradicates cancer by preventing sanitation of the dNTP pool.” Nature 508: 215-221 (2014); Nissink J. W. M. et al. “Mth1 Substrate Recognition—an Example of Specific Promiscuity.” Plos One 11: 51154 (2016); and, Manuel Ellermann et al. “Novel class of potent and selective inhibitors efface MTH1 as broad-spectrum cancer target.” AACR National Meeting Abstract 5226, 2017.

FIG. 2TTTT-2ZZZZ provide non-limiting examples of MDM2 and/or MDM4 Targeting Ligands wherein R represents exemplary points at which the spacer is attached. For additional examples and related ligands, see, Popowicz G. M. et al. “Structures of low molecular weight inhibitors bound to MDMX and MDM2 reveal new approaches for p53-MDMX/MDM2 antagonist drug discovery.” Cell Cycle, 9 (2010); Miyazaki M. et al. “Synthesis and evaluation of novel orally active p53-MDM2 interaction inhibitors.” Bioorg. Med. Chem. 21: 4319-4331 (2013); Miyazaki M. et al. “Discovery of DS-5272 as a promising candidate: A potent and orally active p53-MDM2 interaction inhibitor.” Bioorg Med. Chem. 23: 2360-7 (2015); Holzer P. et al. “Discovery of a Dihydroisoquinolinone Derivative (NVP-CGM097): A Highly Potent and Selective MDM2 Inhibitor Undergoing Phase 1 Clinical Trials in p53 wt Tumors.” J. Med. Chem. 58: 6348-6358 (2015); Gonzalez-Lopez de Turiso F. et al. “Rational Design and Binding Mode Duality of MDM2-p53 Inhibitors.” J. Med. Chem. 56: 4053-4070 (2013); Gessier F. et al. “Discovery of dihydroisoquinolinone derivatives as novel inhibitors of the p53-MDM2 interaction with a distinct binding mode.” Bioorg. Med. Chem. Lett. 25: 3621-3625 (2015); Fry D. C. et al. “Deconstruction of a nutlin: dissecting the binding determinants of a potent protein-protein interaction inhibitor.” ACS Med Chem Lett 4: 660-665 (2013); Ding Q. et al. “Discovery of RG7388 a Potent and Selective p53-MDM2 Inhibitor in Clinical Development.” J. Med. Chem. 56: 5979-5983 (2013); Wang S. et al. “SAR405838: an optimized inhibitor of MDM2-p53 interaction that induces complete and durable tumor regression.” Cancer Res. 74: 5855-5865 (2014); Rew Y. et al. “Discovery of AM-7209 a Potent and Selective 4-Amidobenzoic Acid Inhibitor of the MDM2-p53 Interaction.” J. Med. Chem. 57: 10499-10511 (2014); Bogen S. L. et al. “Discovery of Novel 3 3-Disubstituted Piperidines as Orally Bioavailable Potent and Efficacious HDM2-p53 Inhibitors.” ACS Med. Chem. Lett. 7: 324-329 (2016); and, Sun D. et al. “Discovery of AMG 232 a Potent Selective and Orally Bioavailable MDM2-p53 Inhibitor in Clinical Development.” J. Med. Chem. 57: 1454-1472 (2014).

FIG. 2AAAAA-2EEEEE provide non-limiting examples of PARP1, PARP2, and/or PARP3 Targeting Ligands wherein R represents exemplary points at which the spacer is attached. For additional examples and related ligands, see, Iwashita A. et al. “Discovery of quinazolinone and quinoxaline derivatives as potent and selective poly(ADP-ribose) polymerase-1/2 inhibitors.” Febs Lett. 579: 1389-1393 (2005); the crystal structure PDB 2RCW (PARP complexed with A861695, Park C. H.); the crystal structure PDB 2RD6 (PARP complexed with A861696, Park C. H.); the crystal structure PDB 3GN7; Miyashiro J. et al. “Synthesis and SAR of novel tricyclic quinoxalinone inhibitors of poly(ADP-ribose)polymerase-1 (PARP-1)” Bioorg. Med. Chem. Lett. 19: 4050-4054 (2009); Gandhi V. B. et al. “Discovery and SAR of substituted 3-oxoisoindoline-4-carboxamides as potent inhibitors of poly(ADP-ribose) polymerase (PARP) for the treatment of cancer.” Bioorg. Med. Chem. Lett. 20: 1023-1026 (2010); Penning T. D. et al. “Optimization of phenyl-substituted benzimidazole carboxamide poly(ADP-ribose) polymerase inhibitors: identification of (S)-2-(2-fluoro-4-(pyrrolidin-2-yl)phenyl)-1H-benzimidazole-4-carboxamide (A-966492) a highly potent and efficacious inhibitor.” J. Med. Chem. 53: 3142-3153 (2010); Ye N. et al. “Design, Synthesis, and Biological Evaluation of a Series of Benzo[de][1 7]naphthyridin-7(8H)-ones Bearing a Functionalized Longer Chain Appendage as Novel PARP1 Inhibitors.” J. Med. Chem. 56: 2885-2903 (2013); Patel M. R. et al. “Discovery and Structure-Activity Relationship of Novel 2 3-Dihydrobenzofuran-7-carboxamide and 2 3-Dihydrobenzofuran-3(2H)-one-7-carboxamide Derivatives as Poly(ADP-ribose)polymerase-1 Inhibitors.” J. Med. Chem. 57: 5579-5601 (2014); Thorsell A. G. et al. “Structural Basis for Potency and Promiscuity in Poly(ADP-ribose) Polymerase (PARP) and Tankyrase Inhibitors.” J. Med. Chem. 60:1262-1271 (2012); the crystal structure PDB 4RV6 (“Human ARTD1 (PARP1) catalytic domain in complex with inhibitor Rucaparib”, Karlberg T. et al.); Papeo G. M. E. et al. “Discovery of 2-[1-(4 4-Difluorocyclohexyl)Piperidin-4-Yl]-6-Fluoro-3-Oxo-2 3-Dihydro-1H-Isoindole-4-Carboxamide (Nms-P118): A Potent Orally Available and Highly Selective Parp-1 Inhibitor for Cancer Therapy.” J. Med. Chem. 58: 6875 (2015); Kinoshita T. et al. “Inhibitor-induced structural change of the active site of human poly(ADP-ribose) polymerase.” Febs Lett. 556: 43-46 (2004); and, Gangloff A. R. et al. “Discovery of novel benzo[b][1 4]oxazin-3(4H)-ones as poly(ADP-ribose)polymerase inhibitors.” Bioorg. Med. Chem. Lett. 23: 4501-4505 (2013).

FIG. 2FFFFF-2GGGGG provide non-limiting examples of PARP14 Targeting Ligands wherein R represents exemplary points at which the spacer is attached.

FIG. 2HHHHH provides non-limiting examples of PARP15 Targeting Ligands wherein R represents exemplary points at which the spacer is attached.

FIG. 2IIIII provides non-limiting examples of PDZ domain Targeting Ligands wherein R represents exemplary points at which the spacer(s) are attached.

FIG. 2JJJJJ provides non-limiting examples of Phospholipase A2 domain Targeting Ligands wherein R represents exemplary points at which the spacer is attached.

FIG. 2KKKKK provides non-limiting examples of Protein S100-A7 2WOS Targeting Ligands wherein R represents exemplary points at which the spacer is attached.

FIG. 2LLLLL-2MMMMM provide non-limiting examples of Saposin-B Targeting Ligands wherein R represents exemplary points at which the spacer is attached.

FIG. 2NNNNN-2OOOOO provide non-limiting examples of Sec7 Targeting Ligands wherein R represents exemplary points at which the spacer is attached.

FIG. 2PPPPP-2QQQQQ provide non-limiting examples of SH2 domain of pp60 Src Targeting Ligands wherein R represents exemplary points at which the spacer is attached.

FIG. 2RRRRR provides non-limiting examples of Tank1 Targeting Ligands wherein R represents exemplary points at which the spacer is attached.

FIG. 2SSSSS provides non-limiting examples of Ubc9 SUMO E2 ligase SF6D Targeting Ligands wherein R represents exemplary points at which the spacer is attached.

FIG. 2TTTTT provides non-limiting examples of Src Targenting Ligands, including AP23464, wherein R represents exemplary points at which the spacer is attached.

FIG. 2UUUUU-2XXXXX provide non-limiting examples of Src-AS1 and/or Src AS2 Targeting Ligands wherein R represents exemplary points at which the spacer is attached.

FIG. 2YYYYY provides non-limiting examples of JAK3 Targeting Ligands, including Tofacitinib, wherein R represents exemplary points at which the spacer is attached.

FIG. 2ZZZZZ provides non-limiting examples of ABL Targeting Ligands, including Tofacitinib and Ponatinib, wherein R represents exemplary points at which the spacer is attached.

FIG. 3A-3B provide non-limiting examples of MEK1 Targeting Ligands, including PD318088, Trametinib and G-573, wherein R represents exemplary points at which the spacer is attached.

FIG. 3C provides non-limiting examples of KIT Targeting Ligands, including Regorafenib, wherein R represents exemplary points at which the spacer is attached.

FIG. 3D-3E provide non-limiting examples of HIV Reverse Transcriptase Targeting Ligands, including Efavirenz, Tenofovir, Emtricitabine, Ritonavir, Raltegravir, and Atazanavir, wherein R represents exemplary points at which the spacer is attached.

FIG. 3F-3G provide non-limiting examples of HIV Protease Targeting Ligands, including Ritonavir, Raltegravir, and Atazanavir, wherein R represents exemplary points at which the spacer is attached.

FIG. 3H-3I provide non-limiting examples of KSR1 Targeting Ligands wherein R represents exemplary points at which the spacer is attached.

FIG. 3J-3L provide non-limiting examples of CTNNB1 Targeting Ligands wherein R represents exemplary points at which the spacer is attached. For example, —crystal structure—and (See “Direct Targeting of b-Catenin by a Small Molecule Stimulates Proteasomal Degradation and Suppresses Oncogenic Wnt/b-Catenin Signaling” Cell Rep 2016, 16(1), 28; “Rational Design of Small-Molecule Inhibitors for β-Catenin/T-Cell Factor Protein-Protein Interactions by Bioisostere Replacement” ACS Chem Biol 2013, 8, 524; and “Allosteric inhibitor of β-catenin selectively targets oncogenic Wnt signaling in colon cancer” Sci Rep 2020, 10, 8096).

FIG. 3M provides non-limiting examples of BCL6 Targeting Ligands wherein R represents exemplary points at which the spacer is attached.

FIG. 3N-30 provide non-limiting examples of PAK1 Targeting Ligands wherein R represents exemplary points at which the spacer is attached.

FIG. 3P-3R provide non-limiting examples of PAK4 Targeting Ligands wherein R represents exemplary points at which the spacer is attached.

FIG. 3S-3T provide non-limiting examples of TNIK Targeting Ligands wherein R represents exemplary points at which the spacer is attached.

FIG. 3U provides non-limiting examples of MEN1 Targeting Ligands wherein R represents exemplary points at which the spacer is attached.

FIG. 3V-3W provide non-limiting examples of ERK1 Targeting Ligands wherein R represents exemplary points at which the spacer is attached.

FIG. 3X provides non-limiting examples of IDO1 Targeting Ligands wherein R represents exemplary points at which the spacer is attached.

FIG. 3Y provides non-limiting examples of CBP Targeting Ligands wherein R represents exemplary points at which the spacer is attached.

FIG. 3Z-3SS provide non-limiting examples of MCL1 Targeting Ligands wherein R represents exemplary points at which the spacer is attached. For additional examples and related ligands, see, Tanaka Y. et al “Discovery of potent Mcl-1/Bcl-xL dual inhibitors by using a hybridization strategy based on structural analysis of target proteins.” J. Med. Chem. 56: 9635-9645 (2013); Friberg A. et al. “Discovery of potent myeloid cell leukemia 1 (Mcl-1) inhibitors using fragment-based methods and structure-based design.” J. Med. Chem. 56: 15-30 (2013); Petros A. M. et al “Fragment-based discovery of potent inhibitors of the anti-apoptotic MCL-1 protein.” Bioorg. Med. Chem. Lett. 24: 1484-1488 (2014); Burke J. P. et al. “Discovery of tricyclic indoles that potently inhibit mcl-1 using fragment-based methods and structure-based design.” J. Med. Chem. 58: 3794-3805 (2015); Pelz N. F. et al. “Discovery of 2-Indole-acylsulfonamide Myeloid Cell Leukemia 1 (Mcl-1) Inhibitors Using Fragment-Based Methods.” J. Med. Chem. 59: 2054-2066 (2016); Clifton M. C. et al. “A Maltose-Binding Protein Fusion Construct Yields a Robust Crystallography Platform for MCL1.” Plos One 10: e0125010-e0I25010 (2015); Kotschy A et al. “The MCL1 inhibitor S63845 is tolerable and effective in diverse cancer models. Nature 538:477-482 (2016); EP 2886545 A1 titled “New thienopyrimidine derivatives a process for their preparation and pharmaceutical compositions containing them”; Jeffrey W. Johannes et al. “Structure Based Design of Non-Natural Peptidic Macrocyclic Mcl-1 Inhibitors” ACS Med Chem. Lett. (2017); DOI: 10.1021/acsmedchemlett.6b00464; Bruncko M. et al. “Structure-Guided Design of a Series of MCL-1 Inhibitors with High Affinity and Selectivity.” J. Med. Chem. 58: 2180-2194 (2015); Taekyu Lee et al. “Discovery and biological characterization of potent myeloid cell leukemia-1 inhibitors.” FEBS Letters 591: 240-251 (2017); Chen L. et al. “Structure-Based Design of 3-Carboxy-Substituted 1 2 3 4-Tetrahydroquinolines as Inhibitors of Myeloid Cell Leukemia-1 (Mcl-1).” Org. Biomol. Chem. 14:5505-5510 (2016); US 2016/0068545 titled “Tetrahydronaphthalene derivatives that inhibit mcl-1 protein”; WO 2016207217 A1 titled “Preparation of new bicyclic derivatives as pro-apoptotic agents”; Gizem Akgay et al. “Inhibition of Mcl-1 through covalent modification of a noncatalytic lysine side chain” Nature Chemical Biology 12: 931-936 (2016).

FIG. 3TT provides non-limiting examples of ASHIL Targeting Ligands wherein R represents exemplary points at which the spacer is attached. See for example, the crystal structure PDB 4YNM (“Human ASHIL SET domain in complex with S-adenosyl methionine (SAM)” Rogawski D. S. et al.)

FIG. 3UU-3WW provide non-limiting examples of ATAD2 Targeting Ligands wherein R represents exemplary points at which the spacer is attached. For additional examples and related ligands, see, Chaikuad A. et al. “Structure-based approaches towards identification of fragments for the low-drugability ATAD2 bromodomain” Med Chem Comm 5: 1843-1848 (2014); Poncet-Montange G. et al. “Observed bromodomain flexibility reveals histone peptide- and small molecule ligand-compatible forms of ATAD2.” Biochem. J. 466: 337-346 (2015); Harner M. J. et al. “Fragment-Based Screening of the Bromodomain of ATAD2.” J. Med. Chem. 57: 9687-9692 (2014); Demont E. H. et al. “Fragment-Based Discovery of Low-Micromolar Atad2 Bromodomain Inhibitors.” J. Med. Chem. 58: 5649 (2015); and, Bamborough P. et al. “Structure-Based Optimization of Naphthyridones into Potent Atad2 Bromodomain Inhibitors.” J. Med. Chem. 58: 6151 (2015).

FIG. 3XX-3AAA provide non-limiting examples of BAZ2A and BAZ2B Targeting Ligands wherein R represents exemplary points at which the spacer is attached. For additional examples and related ligands, see, the crystal structure PDB 4CUU (“Human Baz2B in Complex with Fragment-6 N09645” Bradley A. et al.); the crystal structure PDB 5CUA (“Second Bromodomain of Bromodomain Adjacent to Zinc Finger Domain Protein 2B (BAZ2B) in complex with 1-Acetyl-4-(4-hydroxyphenyl)piperazine”. Bradley A. et al.); Ferguson F. M. et al. “Targeting low-drugability bromodomains: fragment based screening and inhibitor design against the BAZ2B bromodomain.” J. Med. Chem. 56: 10183-10187 (2013); Marchand J. R. et al. “Derivatives of 3-Amino-2-methylpyridine as BAZ2B Bromodomain Ligands: In Silico Discovery and in Crystallo Validation.” J. Med. Chem. 59: 9919-9927 (2016); Drouin L. et al. “Structure Enabled Design of BAZ2-ICR A Chemical Probe Targeting the Bromodomains of BAZ2A and BAZ2B.” J. Med. Chem. 58: 2553-2559 (2015); Chen P. et al. “Discovery and characterization of GSK2801 a selective chemical probe for the bromodomains BAZ2A and BAZ2B.” J. Med. Chem. 59:1410-1424 (2016).

FIG. 3BBB provides non-limiting examples of BRD1 Targeting Ligands wherein R represents exemplary points at which the spacer is attached. For additional examples and related ligands, see, the crystal structure PDB 5AME (“the Crystal Structure of the Bromodomain of Human Surface Epitope Engineered Brd1A in Complex with 3D Consortium Fragment 4-Acetyl-Piperazin-2-One Pearce”, N. M. et al.); the crystal structure PDB 5AMF (“Crystal Structure of the Bromodomain of Human Surface Epitope Engineered Brd1A in Complex with 3D Consortium Fragment Ethyl 4 5 6 7-Tetrahydro-1H-Indazole-5-Carboxylate”, Pearce N. M. et al.); the crystal structure PDB 5FG6 (“the Crystal structure of the bromodomain of human BRD1 (BRPF2) in complex with OF-1 chemical probe.”, Tallant C. et al.); Filippakopoulos P. et al. “Histone recognition and large-scale structural analysis of the human bromodomain family.” Cell, 149: 214-231 (2012).

FIG. 3CCC-3EEE provide non-limiting examples of BRD2 Bromodomain 1 Targeting Ligands wherein R represents exemplary points at which the spacer is attached. For additional examples and related ligands, see, the crystal structure PDB 2ydw; the crystal structure PDB 2yek; the crystal structure PDB 4a9h; the crystal structure PDB 4a9f; the crystal structure PDB 4a9i; the crystal structure PDB 4a9m; the crystal structure PDB 4akn; the crystal structure PDB 4alg, and the crystal structure PDB 4uyf.

FIG. 3FFF-3HHH provide non-limiting examples of BRD2 Bromodomain 2 Targeting Ligands wherein R represents exemplary points at which the spacer is attached. For additional examples and related ligands, see, the crystal structure PDB 3oni; Filippakopoulos P. et al. “Selective Inhibition of BET Bromodomains.” Nature 468: 1067-1073 (2010); the crystal structure PDB 4j1p; McLure K. G. et al. “RVX-208: an Inducer of ApoA-I in Humans is a BET Bromodomain Antagonist.” Plos One 8: e83190-e83190 (2013); Baud M. G. et al. “Chemical biology. A bump-and-hole approach to engineer controlled selectivity of BET bromodomain chemical probes” Science 346: 638-641 (2014); Baud M. G. et al. “New Synthetic Routes to Triazolo-benzodiazepine Analogues: Expanding the Scope of the Bump-and-Hole Approach for Selective Bromo and Extra-Terminal (BET) Bromodomain Inhibition” J. Med. Chem. 59: 1492-1500 (2016); Gosmini R. et al. “The Discovery of I-Bet726 (Gsk1324726A) a Potent Tetrahydroquinoline Apoal Up-Regulator and Selective Bet Bromodomain Inhibitor” J. Med. Chem. 57: 8111 (2014); the crystal structure PDB 5EK9 (“Crystal structure of the second bromodomain of human BRD2 in complex with a hydroquinolinone inhibitor”, Tallant C. et al); the crystal structure PDB 5BT5; the crystal structure PDB 5dfd; Baud M. G. et al. “New Synthetic Routes to Triazolo-benzodiazepine Analogues: Expanding the Scope of the Bump-and-Hole Approach for Selective Bromo and Extra-Terminal (BET) Bromodomain Inhibition” J. Med. Chem. 59: 1492-1500 (2016).

FIG. 3III-3JJJ provide non-limiting examples of BRD4 Bromodomain 1 Targeting Ligands wherein R represents exemplary points at which the spacer is attached. For additional examples and related ligands, see, the crystal structure PDB 5WUU and the crystal structure PDB 5F5Z.

FIG. 3KKK-3LLL provide non-limiting examples of BRD4 Bromodomain 2 Targeting Ligands wherein R represents exemplary points at which the spacer is attached. For additional examples and related ligands, see, Chung C. W. et al. “Discovery and Characterization of Small Molecule Inhibitors of the Bet Family Bromodomains” J. Med. Chem. 54: 3827 (2011) and Ran X. et al. “Structure-Based Design of gamma-Carboline Analogues as Potent and Specific BET Bromodomain Inhibitors” J. Med. Chem. 58: 4927-4939 (2015).

FIG. 3MMM provides non-limiting examples of BRDT Targeting Ligands wherein R represents exemplary points at which the spacer is attached. For additional examples and related ligands, see, the crystal structure PDB 4flp and the crystal structure PDB 4kcx.

FIG. 3NNN-3QQQ provide non-limiting examples of BRD9 Targeting Ligands wherein R represents exemplary points at which the spacer is attached. For additional examples and related ligands, see, the crystal structure PDB 4ngn; the crystal structure PDB 4uit; the crystal structure PDB 4uiu; the crystal structure PDB 4uiv; the crystal structure PDB 4z6h; the crystal structure PDB 4z6i; the crystal structure PDB 5e9v; the crystal structure PDB 5eu1; the crystal structure PDB 5f1h; the crystal structure PDB 5fp2, (“Structure-Based Design of an in Vivo Active Selective BRD9 Inhibitor” J. Med. Chem., 2016, 59(10), 4462; and WO2016139361).

FIG. 3RRR provides non-limiting examples of SMARCA4 PB1 and/or SMARCA2 Targeting Ligands wherein R represents exemplary points at which the spacer is attached, A is N or CH, and m is 0 1 2 3 4 5 6 7 or 8.

FIG. 3SSS-3XXX provide non-limiting examples of additional Bromodomain Targeting Ligands wherein R represents exemplary points at which the spacer is attached. For additional examples and related ligands, see, Hewings et al. “3 5-Dimethylisoxazoles Act as Acetyl-lysine Bromodomain Ligands.” J. Med. Chem. 54 6761-6770 (2011); Dawson et al. “Inhibition of BET Recruitment to Chromatin as an Effective Treatment for MLL-fusion Leukemia.” Nature, 478, 529-533 (2011); US 2015/0256700; US 2015/0148342; WO 2015/074064; WO 2015/067770; WO 2015/022332; WO 2015/015318; and, WO 2015/011084.

FIG. 3YYY provides non-limiting examples of PB1 Targeting Ligands wherein R represents exemplary points at which the spacer is attached. For additional examples and related ligands, see, the crystal structure PDB 3mb4; the crystal structure PDB 4q0n; and, the crystal structure PDB 5fh6.

FIG. 3ZZZ provides non-limiting examples of SMARCA4 Targeting Ligands wherein R represents exemplary points at which the spacer is attached. For additional examples and related ligands, see, the crystal structure 3uvd and the crystal structure 5dkd.

FIG. 3AAAA provides non-limiting examples of SMARCA2 Targeting Ligands wherein R represents exemplary points at which the spacer is attached. For additional examples and related ligands, see, the crystal structure 5dkc and the crystal structure 5dkh; and WO2020023657, US20200038378, WO2020010227, WO2020078933, WO2019207538, WO2016138114, WO2020035779, and “Discovery of Orally Active Inhibitors of Brahma Homolog (BRM)/SMARCA2 ATPase Activity for the Treatment of Brahma Related Gene 1 (BRG1)/SMARCA4-Mutant Cancers” J Med Chem 2018, 61, 10155.

FIG. 3BBBB provides non-limiting examples of TRIM24 (TIF1a) and/or BRPF1 Targeting Ligands wherein R represents exemplary points at which the spacer is attached and m is 0 1 2 3 4 5 6 7 or 8.

FIG. 3CCCC provides non-limiting examples of TRIM24 (TIF1a) Targeting Ligands wherein R represents exemplary points at which the spacer is attached. For additional examples and related ligands, see, Palmer W. S. et al. “Structure-Guided Design of IACS-9571: a Selective High-Affinity Dual TRIM24-BRPF1 Bromodomain Inhibitor.” J. Med. Chem. 59: 1440-1454 (2016).

FIG. 3DDDD-3FFFF provide non-limiting examples of BRPF1 Targeting Ligands wherein R represents exemplary points at which the spacer is attached. For additional examples and related ligands, see, the crystal structure PDB 4uye; the crystal structure PDB 5c7n; the crystal structure PDB 5c87; the crystal structure PDB 5c89; the crystal structure PDB 5d7x; the crystal structure PDB 5dya; the crystal structure PDB 5epr; the crystal structure PDB 5eql; the crystal structure PDB 5etb; the crystal structure PDB 5ev9; the crystal structure PDB 5eva; the crystal structure PDB 5ewv; the crystal structure PDB 5eww; the crystal structure PDB 5ffy; the crystal structure PDB 5fg5; and, the crystal structure PDB 5g4r.

FIG. 3GGGG provides non-limiting examples of CECR2 Targeting Ligands wherein R represents exemplary points at which the spacer is attached. For additional examples and related ligands, see, Moustakim M. et al. Med. Chem. Comm. 7:2246-2264 (2016) and Crawford T. et al. Journal of Med. Chem. 59; 5391-5402 (2016).

FIG. 3HHHH-3OOOO provide non-limiting examples of CREBBP Targeting Ligands wherein R represents exemplary points at which the spacer is attached, A is N or CH, and m is 0 1 2 3 4 5 6 7 or 8. For additional examples and related ligands, see, the crystal structure PDB 3p1d; the crystal structure PDB 3svh; the crystal structure PDB 4nr4; the crystal structure PDB 4nr5; the crystal structure PDB 4ts8; the crystal structure PDB 4nr6; the crystal structure PDB 4nr7; the crystal structure PDB 4nyw; the crystal structure PDB 4nyx; the crystal structure PDB 4tqn; the crystal structure PDB 5cgp; the crystal structure PDB 5dbm; the crystal structure PDB 5ep7; the crystal structure PDB 5i83; the crystal structure PDB 5i86; the crystal structure PDB 5i89; the crystal structure PDB 5i8g; the crystal structure PDB 5j0d; the crystal structure PDB 5ktu; the crystal structure PDB 5ktw; the crystal structure PDB 5ktx; the crystal structure PDB 5tb6.

FIG. 3PPPP provides non-limiting examples of EP300 Targeting Ligands wherein R represents exemplary points at which the spacer is attached. For additional examples and related ligands, see, the crystal structure PDB 5BT3.

FIG. 3QQQQ provides non-limiting examples of PCAF Targeting Ligands wherein R represents exemplary points at which the spacer is attached. See for example, M. Ghizzoni et al.

Bioorg. Med. Chem. 18: 5826-5834 (2010).

FIG. 3RRRR provides non-limiting examples of PHIP Targeting Ligands wherein R represents exemplary points at which the spacer is attached. For additional examples and related ligands, see, Mol Cancer Ther. 7(9): 2621-2632 (2008).

FIG. 3SSSS provides non-limiting examples of TAF1 and TAF1L Targeting Ligands wherein R represents exemplary points at which the spacer is attached. For additional examples and related ligands, see, Picaud S. et al. Sci Adv 2: e1600760-e1600760 (2016).

FIG. 3TTTT provides non-limiting examples of Histone Deacetylase 2 (HDAC2) Targeting Ligands wherein R represents exemplary points at which the spacer is attached. For additional examples and related ligands, see, Lauffer B. E. J. Biol. Chem. 288: 26926-26943 (2013); Wagner F. F. Bioorg. Med. Chem. 24: 4008-4015 (2016); Bressi J. C. Bioorg. Med. Chem. Lett. 20: 3142-3145 (2010); and, Lauffer B. E. J. Biol. Chem. 288: 26926-26943 (2013).

FIG. 3UUUU-3VVVV provide non-limiting examples of Histone Deacetylase 4 (HDAC4) Targeting Ligands wherein R represents exemplary points at which the spacer is attached. For additional examples and related ligands, see, Burli R. W. J. Med. Chem. 56: 9934 (2013); Luckhurst C. A. ACS Med Chem. Lett. 7: 34 (2016); Bottomley M. J. J. Biol. Chem. 283: 26694-26704 (2008).

FIG. 3WWWW provides non-limiting examples of Histone Deacetylase 6 Targeting Ligands wherein R represents exemplary points at which the spacer is attached. For additional examples and related ligands, see, Harding R. J. (to be published); Hai Y. Nat. Chem. Biol. 12: 741-747, (2016); and, Miyake Y. Nat. Chem. Biol. 12: 748 (2016).

FIG. 3XXXX-3YYYY provide non-limiting examples of Histone Deacetylase 7 Targeting Ligands wherein R represents exemplary points at which the spacer is attached. For additional examples and related ligands, see, Lobera M. Nat. Chem. Biol. 9: 319 (2013) and Schuetz A. J. Biol. Chem. 283: 11355-11363 (2008).

FIG. 3ZZZZ-3DDDDD provide non-limiting examples of Histone Deacetylase 8 Targeting Ligands wherein R represents exemplary points at which the spacer is attached. For additional examples and related ligands, see, Whitehead L. Biol. Med. Chem. 19: 4626-4634 (2011); Tabackman A. A. J. Struct. Biol. 195: 373-378 (2016); Dowling D. P. Biochemistry 47, 13554-13563 (2008); Somoza J. R. Biochemistry 12, 1325-1334 (2004); Decroos C. Biochemistry 54: 2126-2135 (2015); Vannini A. Proc. Natl Acad Sci. 101: 15064 (2004); Vannini A. EMBO Rep. 8: 879 (2007); the crystal structure PDB 5BWZ; Decroos A. ACS Chem. Biol. 9: 2157-2164 (2014); Somoza J. R. Biochemistry 12: 1325-1334 (2004); Decroos C. Biochemistry 54: 6501-6513 (2015); Decroos A. ACS Chem. Biol. 9: 2157-2164 (2014); and, Dowling D. P. Biochemistry 47: 13554-13563 (2008).

FIG. 3EEEEE provides non-limiting examples of Histone Acetyltransferase (KAT2B) Targeting Ligands wherein R represents exemplary points at which the spacer is attached. For additional examples and related ligands, see, Chaikuad A. J. Med. Chem. 59: 1648-1653 (2016); the crystal structure PDB 1ZS5; and, Zeng L. J. Am. Chem. Soc. 127: 2376-2377 (2005).

FIG. 3FFFFF-3GGGGG provide non-limiting examples of Histone Acetyltransferase (KAT2A) Targeting Ligands wherein R represents exemplary points at which the spacer is attached. For additional examples and related ligands, see, Ringel A. E. Acta Crystallogr. D. Struct. Biol. 72: 841-848 (2016).

FIG. 3HHHHH provides non-limiting examples of Histone Acetyltransferase Type B Catalytic Unit (HAT1) Targeting Ligands wherein R represents exemplary points at which the spacer is attached. For additional examples and related ligands, see, the crystal structure PDB 2POW.

FIG. 3IIIII provides non-limiting examples of Cyclic AMP-dependent Transcription Factor (ATF2) Targeting Ligands wherein R represents exemplary points at which the spacer is attached.

FIG. 3JJJJJ provides non-limiting examples of Histone Acetyltransferase (KAT5) Targeting Ligands wherein R represents exemplary points at which the spacer is attached.

FIG. 3KKKKK-3MMMMM provide non-limiting examples of Lysine-specific histone demethylase 1A (KDM1A) Targeting Ligands wherein R represents exemplary points at which the spacer is attached. For additional examples and related ligands, see, Mimasu S. Biochemistry 49: 6494-6503 (2010); Sartori L. J. Med. Chem. 60:1673-1693 (2017); and, Vianello P. J. Med. Chem. 60: 1693-1715 (2017).

FIG. 3NNNNN provides non-limiting examples of HDAC6 Zn Finger Domain Targeting Ligands wherein R represents exemplary points at which the spacer is attached.

FIG. 3OOOOO-3PPPPP provide non-limiting examples of general Lysine Methyltransferase Targeting Ligands wherein R represents exemplary points at which the spacer is attached.

FIG. 3QQQQQ-3TTTTT provide non-limiting examples of DOT1L Targeting Ligands wherein R represents exemplary points at which the spacer is attached, A is N or CH, and m is 0 1 2 3 4 5 6 7 or 8. For additional examples and related ligands, see, the crystal structure PDB 5MVS (“Dot1L in complex with adenosine and inhibitor CPD1” Mobitz, H. et al., ACS Med Chem Lett., 2017, 8: 338-343); the crystal structure PDB 5MW3, 5MW4 (“Dot1L in complex inhibitor CPD7” Be C. et al.); the crystal structure PDB 5DRT (“Dot1L in complex inhibitor CPD2” Chen, C., et al., ACS Med Chem Lett., 2016, 7: 735-740); the crystal structure PDB 5DRY (“Dot1L in complex with CPD3”, Chen, C., et al., ACS Med Chem Lett., 2016, 7: 735-740), the crystal structure of PDB 5DSX (“Dot1L in complex with CPD10”, Chen, C., et al., ACS Med Chem Lett., 2016, 7: 735-740), the crystal structure PDB 5DT2 (“Dot1L in complex with CPD11”, Chen, C., et al., ACS Med Chem Lett., 2016, 7: 735-740), the crystal structure PDB 5JUW “(Dot1L in complex with SS148” Yu W. et al. Structural Genomics Consortium), the crystal structure PDB 6TE6 (“Dot1L in complex with an inhibitor, compound 3”, Stauffer, F., et al., ACS Med Chem Lett., 2019, 10: 1655-1660).

FIG. 3UUUUU provides non-limiting examples of EHMT1 Targeting Ligands wherein R represents exemplary points at which the spacer is attached. For additional examples and related ligands, see, the crystal structure PDB 5TUZ (“EHMT1 in complex with inhibitor MS0124”, Babault N. et al.).

FIG. 3VVVVV provides non-limiting examples of EHMT2 Targeting Ligands wherein R represents exemplary points at which the spacer is attached. For additional examples and related ligands, see, the crystal structure PDB 5TUY (“EHMT2 in complex with inhibitor MS0124”, Babault N. et al.); the PDB crystal structure 5TTF (“EHMT2 in complex with inhibitor MS012”, Dong A. et al.); the PDB crystal structure 3RJW (Dong A. et al., Structural Genomics Consortium); the PDB crystal structure 3K5K; Liu F. et al. J. Med. Chem. 52: 7950-7953 (2009); and, the PDB crystal structure 4NVQ (“EHMT2 in complex with inhibitor A-366” Sweis R. F. et al.).

FIG. 3WWWWW provides non-limiting examples of SETD2 Targeting Ligands wherein R represents exemplary points at which the spacer is attached. For additional examples and related ligands, see, the PDB crystal structure 5LSY (“SETD2 in complex with cyproheptadine”, Tisi D. et al.); Tisi D. et al. ACS Chem. Biol. 11: 3093-3105 (2016); the crystal structures PDB 5LSS, 5LSX, 5LSZ, 5LT6, 5LT7, and 5LT8; the PDB crystal structure 4FMU; and, Zheng W. et al. J. Am. Chem. Soc. 134: 18004-18014 (2012).

FIG. 3XXXXX-3YYYYY provide non-limiting examples of SETD7 Targeting Ligands wherein R represents exemplary points at which the spacer is attached. For additional examples and related ligands, see, the PDB crystal structure 5AYF (“SETD7 in complex with cyproheptadine.” Niwa H. et al.); the PDB crystal structure 4JLG (“SETD7 in complex with (R)-PFI-2”, Dong A. et al.); the PDB crystal structure 4JDS (Dong A. et. al Structural Genomics Consortium); the PDB crystal structure 4E47 (Walker J. R. et al. Structural Genomics Consortium; the PDB crystal structure 3VUZ (“SETD7 in complex with AAM-1.” Niwa H. et al.); the PDB crystal structure 3VVO; and, Niwa H et al. Acta Crystallogr. Sect. D 69: 595-602 (2013).

FIG. 3ZZZZZ provides non-limiting examples of SETD8 Targeting Ligands wherein R represents exemplary points at which the spacer is attached. For additional examples and related ligands, see, the PDB crystal structure 5TH7 (“SETD8 in complex with MS453”, Yu W. et al.) and the PDB crystal structure 5T5G (Yu W et. al.; to be published).

FIG. 4A-4B provides non-limiting examples of SETDB1 Targeting Ligands wherein R represents exemplary points at which the spacer is attached. For additional examples and related ligands, see, the PDB crystal structure 5KE2 (“SETDB1 in complex with inhibitor XST06472A”, Iqbal A. et al.); the PDB crystal structure 5KE3 (“SETDB1 in complex with fragment MRT0181a”, Iqbal A. et al.); the PDB crystal structure 5KH6 (“SETDB1 in complex with fragment methyl 3-(methylsulfonylamino)benzoate”, Walker J. R. et al. Structural Genomics Consortium); and, the PDB crystal structure 5KCO (“SETDB1 in complex with [N]-(4-chlorophenyl)methanesulfonamide”, Walker J. R. et al.)

FIG. 4C-4P provides non-limiting examples of SMYD2 Targeting Ligands wherein R represents exemplary points at which the spacer is attached. For additional examples and related ligands, see, the PDB crystal structure 5KJK (“SMYD2 in complex with inhibitor AZ13450370”, Cowen S. D. et al.); the PDB crystal structure 5KJM (“SMYD2 in complex with AZ931”, Cowen S. D. et al.); the PDB crystal structure 5KJN (“SMYD2 in complex with AZ506”, Cowen S. D. et al.); the PDB crystal structure 5ARF (“SMYD2 in complex with N-[3-(4-chlorophenyl)-1-{N′-cyano-N-[3-(difluoromethoxy)phenyl]carbamimidoyl}-4 5-dihydro-1H-pyrazol-4-YL]-N-ethyl-2-hydroxyacetamide”, Eggert E. et al.); the PDB crystal structure 5ARG (“SMYD2 in complex with BAY598”, Eggert E. et al.); the PDB crystal structure 4YND (“SMYD2 in complex with A-893”, Sweis R. F. et al.); the PDB crystal structure 4WUY (“SMYD2 in complex with LLY-507”, Nguyen H. et al.); and, the PDB crystal structure 3S7B (“N-cyclohexyl-N˜3˜˜[2-(3 4-dichlorophenyl)ethyl]-N-(2-{[2-(5-hydroxy-3-oxo-3 4-dihydro-2H-1 4-benzoxazin-8-yl)ethyl]amino}ethyl)-beta-alaninamide”, Ferguson A. D. et al.).

FIG. 4Q-4R provide non-limiting examples of SMYD3 Targeting Ligands wherein R represents exemplary points at which the spacer is attached. For additional examples and related ligands, see, the crystal structure 5H17 (“SMYD3 in complex with 5′-{[(3S)-3-amino-3-carboxypropyl][3-(dimethylamino)propyl]amino}-5′-deoxyadenosine”, Van Aller G. S. et al.); the crystal structure 5CCL (“SMYD3 in complex with oxindole compound”, Mitchell L. H. et al.); and, the crystal structure 5CCM (“Crystal structure of SMYD3 with SAM and EPZ030456”).

FIG. 4S provides non-limiting examples of SUV4-20H1 Targeting Ligands wherein R represents exemplary points at which the spacer is attached. For additional examples and related ligands, see, the PDB crystal structure 5CPR (“SUV4-20H1 in complex with inhibitor A-196”, Bromberg K. D. et al.).

FIG. 4T-4AA provide non-limiting examples of Wild Type Androgen Receptor Targeting Ligands wherein R represents exemplary points at which the spacer is attached. For additional examples and related ligands, see, the PDB crystal structures 5T8E and 5T8J (“Androgen Receptor in complex with 4-(pyrrolidin-1-yl)benzonitrile derivatives”, Asano M. et al.); Asano M. et al. Bioorg. Med. Chem. Lett. 27: 1897-1901 (2017); the PDB crystal structure 5JJM (“Androgen Receptor”, Nadal M. et al.); the PDB crystal structure 5CJ6 (“Androgen Receptor in complex with 2-Chloro-4-[[(1R 2R)-2-hydroxy-2-methyl-cyclopentyl]amino]-3-methyl-benzonitrile derivatives”, Saeed A. et al.); the PDB crystal structure 4QL8 (“Androgen Receptor in complex with 3-alkoxy-pyrrolo[1 2-b]pyrazolines derivatives”, Ullrich T. et al.); the PDB crystal structure 4HLW (“Androgen Receptor Binding Function 3 (BF3) Site of the Human Androgen Receptor through Virtual Screening”, Munuganti R. S. et al.); the PDB crystal structure 3V49 (“Androgen Receptor lbd with activator peptide and sarm inhibitor 1”, Nique F. et al.); Nique F. et al. J. Med. Chem. 55: 8225-8235 (2012); the PDB crystal structure 2YHD (“Androgen Receptor in complex with AF2 small molecule inhibitor”, Axerio-Cilies P. et al.); the PDB crystal structure 3RLJ (“Androgen Receptor ligand binding domain in complex with SARM S-22”, Bohl C. E. et al.); Bohl C. E. et al. J. Med. Chem. 54: 3973-3976 (2011); the PDB crystal structure 3B5R (“Androgen Receptor ligand binding domain in complex with SARM C-31”, Bohl C. E. et al.); Bohl C. E. et al. Bioorg. Med. Chem. Lett. 18: 5567-5570 (2008); the PDB crystal structure 2PIP (“Androgen Receptor ligand binding domain in complex with small molecule”, Estebanez-Perpina E. et al.); Estebanez-Perpina. E. Proc. Natl. Acad Sci. 104:16074-16079 (2007); the PDB crystal structure 2PNU (“Androgen Receptor ligand binding domain in complex with EM5744”, Cantin L. et al.); and, the PDB crystal structure 2HVC (“Androgen Receptor ligand binding domain in complex with LGD2226”, Wang F. et al.). For additional related ligands, see, Matias P. M. et al. “Structural Basis for the Glucocorticoid Response in a Mutant Human Androgen Receptor (Ar(Ccr)) Derived from an Androgen-Independent Prostate Cancer.” J. Med. Chem. 45: 1439 (2002); Sack J. S. et al. “Crystallographic structures of the ligand-binding domains of the androgen receptor and its T877A mutant complexed with the natural agonist dihydrotestosterone.” Proc. Natl. Acad Sci. 98: 4904-4909 (2001); He B. et al. “Structural basis for androgen receptor interdomain and coactivator interactions suggests a transition in nuclear receptor activation function dominance.” Mol. Cell 16: 425-438 (2004); Pereira de Jesus-Tran K. “Comparison of crystal structures of human androgen receptor ligand-binding domain complexed with various agonists reveals molecular determinants responsible for binding affinity.” Protein Sci. 15: 987-999 (2006); Bohl C. E. et al. “Structural Basis for Accommodation of Nonsteroidal Ligands in the Androgen Receptor.” Mol Pharmacol. 63(1):211-23 (2003); Sun C. et al. “Discovery of potent orally-active and muscle-selective androgen receptor modulators based on an N-aryl-hydroxybicyclohydantoin scaffold.” J. Med. Chem. 49: 7596-7599 (2006); Nirschl A. A. et al. “N-aryl-oxazolidin-2-imine muscle selective androgen receptor modulators enhance potency through pharmacophore reorientation.” J Med Chem. 52: 2794-2798 (2009); Bohl C. E. et al. “Effect of B-ring substitution pattern on binding mode of propionamide selective androgen receptor modulators.” Bioorg. Med. Chem. Lett. 18: 5567-5570 (2008); Ullrich T. et al. “3-alkoxy-pyrrolo[1 2-b]pyrazolines as selective androgen receptor modulators with ideal physicochemical properties for transdermal administration.” J. Med. Chem. 57: 7396-7411 (2014); Saeed A. et al. “2-Chloro-4-[[(1R 2R)-2-hydroxy-2-methyl-cyclopentyl]amino]-3-methyl-benzonitrile: A Transdermal Selective Androgen Receptor Modulator (SARM) for Muscle Atrophy.” J. Med. Chem. 59: 750-755 (2016); Nique et al. “Discovery of diarylhydantoins as new selective androgen receptor modulators.” J. Med. Chem. 55: 8225-8235 (2012); and, Michael E. Jung et al. “Structure-Activity Relationship for Thiohydantoin Androgen Receptor Antagonists for Castration-Resistant Prostate Cancer (CRPC).” J. Med. Chem. 53: 2779-2796 (2010).

FIG. 4BB provides non-limiting examples of Mutant T877A Androgen Receptor Targeting Ligands wherein R represents exemplary points at which the spacer is attached. For additional examples and related ligands, see, the PDB crystal structure 40GH (‘Androgen Receptor T877A-AR-LBD”, Hsu C. L. et al.) and the PDB crystal structure 20Z7 (“Androgen Receptor T877A-AR-LBD”, Bohl C. E. et al.).

FIG. 4CC provides non-limiting examples of Mutant W741L Androgen Receptor Targeting Ligands wherein R represents exemplary points at which the spacer is attached. For additional examples and related ligands, see, the PDB crystal structure 40JB (“Androgen Receptor T877A-AR-LBD”, Hsu C. L. et al.).

FIG. 4DD-4EE provide non-limiting examples of Estrogen and/or Androgen Targeting Ligands wherein R represents exemplary points at which the spacer is attached.

FIG. 5A provides non-limiting examples of Afatinib, a Targeting Ligands for the EGFR and ErbB2/4 receptors. R represents exemplary points at which the spacer is attached.

FIG. 5B provides non-limiting examples of Axitinib, a Targeting Ligands for the VEGFR1/2/3, PDGFRP, and Kit receptors. R represents exemplary points at which the spacer is attached.

FIG. 5C-5D provide non-limiting examples of Bosutinib, a Targeting Ligands for the BCR-Abl, Src, Lyn and Hck receptors. R represents exemplary points at which the spacer is attached.

FIG. 5E provides non-limiting examples of Cabozantinib, a Targeting Ligands for the RET, c-Met, VEGFR1/2/3, Kit, TrkB, Flt3, Axl, and Tie 2 receptors. R represents exemplary points at which the spacer is attached.

FIG. 5F provides non-limiting examples of Ceritinib, a Targeting Ligands for the ALK, IGF-1R, InsR, and ROS1 receptors. R represents exemplary points at which the spacer is attached.

FIG. 5G provides non-limiting examples of Crizotinib, a Targeting Ligands for the ALK, c-Met, HGFR, ROS1, and MST1R receptors. R represents exemplary points at which the spacer is attached.

FIG. 5H provides non-limiting examples of Dabrafenib, a Targeting Ligands for the B-Raf receptor. R represents exemplary points at which the spacer is attached.

FIG. 5I provides non-limiting examples of Dasatinib, a Targeting Ligands for the BCR-Abl, Src, Lck, Lyn, Yes, Fyn, Kit, EphA2, and PDGFRP receptors. R represents exemplary points at which the spacer is attached.

FIG. 5J provides non-limiting examples of Erlotinib, a Targeting Ligands for the EGFR receptor. R represents exemplary points at which the spacer is attached.

FIG. 5K-5M provide non-limiting examples of Everolimus, a Targeting Ligands for the HER2 breast cancer receptor, the PNET receptor, the RCC receptors, the RAML receptor, and the SEGA receptor. R represents exemplary points at which the spacer is attached.

FIG. 5N provides non-limiting examples of Gefitinib, a Targeting Ligands for the EGFR and PDGFR receptors. R represents exemplary points at which the spacer is attached.

FIG. 5O provides non-limiting examples of Ibrutinib, a Targeting Ligands for the BTK receptor. R represents exemplary points at which the spacer is attached.

FIG. 5P-5Q provide non-limiting examples of Imatinib, a Targeting Ligands for the BCR-Abl, Kit, and PDGFR receptors. R represents exemplary points at which the spacer is attached.

FIG. 5R-5S provide non-limiting examples of Lapatinib, a Targeting Ligands for the EGFR and ErbB2 receptors. R represents exemplary points at which the spacer is attached.

FIG. 5T provides non-limiting examples of Lenvatinib, a Targeting Ligands for the VEGFR1/2/3, FGFR1/2/3/4, PDGFRα, Kit, and RET receptors. R represents exemplary points at which the spacer is attached.

FIG. 5U-5V provide non-limiting examples of Nilotinib, a Targeting Ligands for the BCR-Abl, PDGRF, and DDR1 receptors. R represents exemplary points at which the spacer is attached.

FIG. 5W-5X provide non-limiting examples of Nintedanib, a Targeting Ligands for the FGFR1/2/3, Flt3, Lck, PDGFRα/β, and VEGFR1/2/3 receptors. R represents exemplary points at which the spacer is attached.

FIG. 5Y-5Z provide non-limiting examples of Palbociclib, a Targeting Ligands for the CDK4/6 receptor. R represents exemplary points at which the spacer is attached.

FIG. 5AA provides non-limiting examples of Pazopanib, a Targeting Ligands for the VEGFR1/2/3, PDGFRα/β, FGFR1/3, Kit, Lck, Fms, and Itk receptors. R represents exemplary points at which the spacer is attached.

FIG. 5BB-5CC provide non-limiting examples of Ponatinib, a Targeting Ligands for the BCR-Abl, T315I VEGFR, PDGFR, FGFR, EphR, Src family kinases, Kit, RET, Tie2, and Flt3 receptors. R represents exemplary points at which the spacer is attached.

FIG. 5DD provides non-limiting examples of Regorafenib, a Targeting Ligands for the VEGFR1/2/3, BCR-Abl, B-Raf, B-Raf (V600E), Kit, PDGFRα/β, RET, FGFR1/2, Tie2, and Eph2A. R represents exemplary points at which the spacer is attached.

FIG. 5EE provides non-limiting examples of Ruxolitinib, a Targeting Ligands for the JAK1/2 receptors. R represents exemplary points at which the spacer is attached.

FIG. 5FF-5GG provide non-limiting examples of Sirolimus, a Targeting Ligands for the FKBP12/mTOR receptors. R represents exemplary points at which the spacer is attached.

FIG. 5HH provides non-limiting examples of Sorafenib, a Targeting Ligands for the B-Raf, CDK8, Kit, Flt3, RET, VEGFR1/2/3, and PDGFR receptors. R represents exemplary points at which the spacer is attached.

FIG. 5II-5JJ provide non-limiting examples of Sunitinib, a Targeting Ligands for PDGFRα/β, VEGFR1/2/3, Kit, Flt3, CSF-1R, RET. R represents exemplary points at which the spacer is attached.

FIG. 5KK-5LL provide non-limiting examples of Temsirolimus, a Targeting Ligands FKBP12/mTOR. R represents exemplary points at which the spacer is attached.

FIG. 5MM provides non-limiting examples of Tofacitinib, a Targeting Ligands for JAK3 receptors. R represents exemplary points at which the spacer is attached.

FIG. 5NN provides non-limiting examples of Trametinib, a Targeting Ligands for the MEK1/2 receptors. R represents exemplary points at which the spacer is attached.

FIG. 5OO-5PP provide non-limiting examples of Vandetanib, a Targeting Ligands for the EGFR, VEGFR, RET, Tie2, Brk, and EphR. R represents exemplary points at which the spacer is attached.

FIG. 5QQ provides non-limiting examples of Vemurafenib, a Targeting Ligands for the A/B/C-Raf, KSR1, and B-Raf (V600E) receptors. R represents exemplary points at which the spacer is attached.

FIG. 5RR provides non-limiting examples of Idelasib, a Targeting Ligands for the PI3Ka receptor. R represents exemplary points at which the spacer is attached.

FIG. 5SS provides non-limiting examples of Buparlisib, a Targeting Ligands for the PI3Ka receptor. R represents exemplary points at which the spacer is attached.

FIG. 5TT provides non-limiting examples of Taselisib, a Targeting Ligands for the PI3Ka receptor. R represents exemplary points at which the spacer is attached.

FIG. 5UU provides non-limiting examples of Copanlisib, a Targeting Ligands for the PI3Ka. R represents exemplary points at which the spacer is attached.

FIG. 5VV provides non-limiting examples of Alpelisib, a Targeting Ligands for the PI3Ka. R represents exemplary points at which the spacer is attached.

FIG. 5WW provides non-limiting examples of Niclosamide, a Targeting Ligands for the CNNTB1. R represents exemplary points at which the spacer is attached.

FIG. 6A-6B provide nonlimiting examples of the BRD4 Bromodomains of PCAF and GCN5 receptors 1 Targeting Ligands wherein R represents exemplary points at which the spacer is attached. For additional examples and related ligands, see, the PDB crystal structure 5tpx (“Discovery of a PCAF Bromodomain Chemical Probe”); Moustakim, M., et al. Angew. Chem. Int. Ed. Engl. 56: 827 (2017); the PDB crystal structure 5mlj (“Discovery of a Potent, Cell Penetrant, and Selective p300/CBP-Associated Factor (PCAF)/General Control Nonderepressible 5 (GCN5) Bromodomain Chemical Probe”); and, Humphreys, P. G. et al. J. Med. Chem. 60: 695 (2017).

FIG. 6C-6D provide nonlimiting examples of G9a (EHMT2) Targeting Ligands wherein R represents exemplary points at which the spacer is attached. For additional examples and related ligands, see, the PDB crystal structure 3k5k; (“Discovery of a 2,4-diamino-7-aminoalkoxyquinazoline as a potent and selective inhibitor of histone lysine methyltransferase G9a”); Liu, F. et al. J. Med. Chem. 52: 7950 (2009); the PDB crystal structure 3rjw (“A chemical probe selectively inhibits G9a and GLP methyltransferase activity in cells”); Vedadi, M. et al. Nat. Chem. Biol. 7: 566 (2011); the PDB crystal structure 4nvq (“Discovery and development of potent and selective inhibitors of histone methyltransferase g9a”); and, Sweis, R. F. et al. ACS Med Chem Lett 5: 205 (2014).

FIG. 6E-6G provide nonlimiting examples of EZH2 Targeting Ligands wherein R represents exemplary points at which the spacer is attached. For additional examples and related ligands, see, the PDB crystal structure 5ij8 (“Polycomb repressive complex 2 structure with inhibitor reveals a mechanism of activation and drug resistance”); Brooun, A. et al. Nat Commun 7: 11384 (2016); the PDB crystal structure 51s6 (“Identification of (R)—N-((4-Methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide (CPI-1205), a Potent and Selective Inhibitor of Histone Methyltransferase EZH2, Suitable for Phase I Clinical Trials for B-Cell Lymphomas”); Vaswani, R. G. et al. J. Med. Chem. 59: 9928 (2016); and, the PDB crystal structures 5ij8 and 51s6.

FIG. 6H-6I provide non-limiting examples of EED Targeting Ligands wherein R represents exemplary points at which the spacer is attached. For additional examples and related ligands, see, the PDB crystal structures 5h15 and 5h19 (“Discovery and Molecular Basis of a Diverse Set of Polycomb Repressive Complex 2 Inhibitors Recognition by EED”); Li, L. et al. PLoS ONE 12: e0169855 (2017); and, the PDB crystal structure 5h19.

FIG. 6J provides non-limiting examples of KMT5A (SETD8) Targeting Ligands wherein R represents exemplary points at which the spacer is attached. See for example, the PDB crystal structure 5t5g.

FIG. 6K-6L provide non-limiting examples of DOTIL Targeting Ligands wherein R represents exemplary points at which the spacer is attached. For additional examples and related ligands, see, the PDB crystal structure 4eki (“Conformational adaptation drives potent, selective and durable inhibition of the human protein methyltransferase DOT1L”); Basavapathruni, A. et al. Chem. Biol. Drug Des. 80: 971 (2012); the PDB crystal structure 4hra (“Potent inhibition of DOT1L as treatment of MLL-fusion leukemia”); Daigle, S. R. et al. Blood 122: 1017 (2013); the PDB crystal structure 5dry (“Discovery of Novel Dot1L Inhibitors through a Structure-Based Fragmentation Approach”) Chen, C. et al. ACS Med. Chem. Lett. 7: 735 (2016); the PDB crystal structure 5dt2 (“Discovery of Novel Dot1L Inhibitors through a Structure-Based Fragmentation Approach”); and, Chen, C. et al. ACS Med. Chem. Lett. 7: 735 (2016).

FIG. 6M-6N provide nonlimiting examples of PRMT3 Targeting Ligands wherein R represents exemplary points at which the spacer is attached. For additional examples and related ligands, see, the PDB crystal structure 3smq (“An allosteric inhibitor of protein arginine methyltransferase 3”); Siarheyeva, A. et al. Structure 20: 1425 (2012); PDB crystal structure 4ryl (“A Potent, Selective and Cell-Active Allosteric Inhibitor of Protein Arginine Methyltransferase 3 (PRMT3)”); and Kaniskan, H. U. et al. Angew. Chem. Int. Ed. Engl. 54: 5166 (2015).

FIG. 6O provides non-limiting examples of CARM1 (PRMT4) Targeting Ligands wherein R represents exemplary points at which the spacer is attached. For additional examples and related ligands, see, the PDB crystal structures 2y1x and 2y1w and related ligands described in “Structural Basis for Carm1 Inhibition by Indole and Pyrazole Inhibitors.” Sack, J. S. et al. Biochem. J. 436: 331 (2011).

FIG. 6P provides non-limiting examples of PRMT5 Targeting Ligands wherein R represents exemplary points at which the spacer is attached. For additional examples and related ligands, see, the PDB crystal structure 4x61 and related ligands described in “A selective inhibitor of PRMT5 with in vivo and in vitro potency in MCL models”. Chan-Penebre, E. Nat. Chem. Biol. 11: 432 (2015).

FIG. 6Q provides non-limiting examples of PRMT6 Targeting Ligands wherein R represents exemplary points at which the spacer is attached. For additional examples and related ligands, see, the PDB crystal structure 4y30 and related ligands described in “Aryl Pyrazoles as Potent Inhibitors of Arginine Methyltransferases: Identification of the First PRMT6 Tool Compound”. Mitchell, L. H. et al. ACS Med. Chem. Lett. 6: 655 (2015).

FIG. 6R provides non-limiting examples of LSD1 (KDM1A) Targeting Ligands wherein R represents exemplary points at which the spacer is attached. For additional examples and related ligands, see, the PDB crystal structure 51gu and related ligands described in “Thieno[3,2-b]pyrrole-5-carboxamides as New Reversible Inhibitors of Histone Lysine Demethylase KDM1A/LSD1. Part 2: Structure-Based Drug Design and Structure-Activity Relationship”. Vianello, P. et al. J. Med. Chem. 60: 1693 (2017).

FIG. 6S-6T provides non-limiting examples of KDM4 Targeting Ligands wherein R represents exemplary points at which the spacer is attached. For additional examples and related ligands, see, the PDB crystal structure 3rvh; the PDB crystal structure 5a7p and related ligands described in “Docking and Linking of Fragments to Discover Jumonji Histone Demethylase Inhibitors.” Korczynska, M., et al. J. Med. Chem. 59: 1580 (2016); and, the PDB crystal structure 3f3c and related ligands described in “8-Substituted Pyrido[3,4-d]pyrimidin-4(3H)-one Derivatives As Potent, Cell Permeable, KDM4 (JMJD2) and KDM5 (JARID1) Histone Lysine Demethylase Inhibitors.” Bavetsias, V. et al. J. Med. Chem. 59: 1388 (2016).

FIG. 6U provides non-limiting examples of KDM5 Targeting Ligands wherein R represents exemplary points at which the spacer is attached. For additional examples and related ligands, see, the PDB crystal structure 3fun and related ligands described in “Structural Analysis of Human Kdm5B Guides Histone Demethylase Inhibitor Development”. Johansson, C. et al. Nat. Chem. Biol. 12: 539 (2016) and the PDB crystal structure 5ceh and related ligands described in “An inhibitor of KDM5 demethylases reduces survival of drug-tolerant cancer cells”. Vinogradova, M. et al. Nat. Chem. Biol. 12: 531 (2016).

FIG. 6V-6W provide non-limiting examples of KDM6 Targeting Ligands wherein R represents exemplary points at which the spacer is attached. For additional examples and related ligands, see, the PDB crystal structure 4ask and related ligands described in “A Selective Jumonji H3K27 Demethylase Inhibitor Modulates the Proinflammatory Macrophage Response”. Kruidenier, L. et al. Nature 488: 404 (2012).

FIG. 6X provides non-limiting examples of L3MBTL3 targeting ligands wherein R represents exemplary points at which the spacer is attached. See for example, the PDB crystal structure 4fl6.

FIG. 6Y provides non-limiting examples of Menin Targeting Ligands wherein R represents exemplary points at which the spacer is attached. For additional examples and related ligands, see, the PDB crystal structure 4x5y and related ligands described in “Pharmacologic Inhibition of the Menin-MLL Interaction Blocks Progression of MLL Leukemia In Vivo” Borkin, D. et al. Cancer Cell 27: 589 (2015) and the PDB crystal structure 4og8 and related ligands described in “High-Affinity Small-Molecule Inhibitors of the Menin-Mixed Lineage Leukemia (MLL) Interaction Closely Mimic a Natural Protein-Protein Interaction” He, S. et al. J. Med. Chem. 57: 1543 (2014).

FIG. 6Z-6AA provide non-limiting examples of HDAC6 Targeting Ligands wherein R represents exemplary points at which the spacer is attached. See for example, the PDB crystal structures 5kh3 and 5eei.

FIG. 6BB provides non-limiting examples of HDAC7 Targeting Ligands wherein R represents exemplary points at which the spacer is attached. For additional examples and related ligands, see, the PDB crystal structure 3c10 and related ligands described in “Human HDAC7 harbors a class IIa histone deacetylase-specific zinc binding motif and cryptic deacetylase activity.” Schuetz, A. et al. J. Biol. Chem. 283: 11355 (2008) and the PDB crystal structure PDB 3zns and related ligands described in “Selective Class Iia Histone Deacetylase Inhibition Via a Non-Chelating Zinc Binding Group”. Lobera, M. et al. Nat. Chem. Biol. 9: 319 (2013).

FIG. 7A-7C provide non-limiting examples of Protein Tyrosine Phosphatase, Non-Receptor Type 1, PTP1B Targeting Ligands wherein R represents exemplary points at which the spacer is attached. For additional examples and related ligands, see, the PDB crystal structure 1bzj described in “Structural basis for inhibition of the protein tyrosine phosphatase 1B by phosphotyrosine peptide mimetics” Groves, M. R. et al. Biochemistry 37: 17773-17783 (1998); the PDB crystal structure 3cwe described in “Discovery of [(3-bromo-7-cyano-2-naphthyl)(difluoro)methyl]phosphonic acid, a potent and orally active small molecule PTP1B inhibitor”. Han Y, Bioorg Med Chem Lett. 18:3200-5 (2008); the PDB crystal structures 2azr and 2b07 described in “Bicyclic and tricyclic thiophenes as protein tyrosine phosphatase 1B inhibitors.” Moretto, A. F. et al. Bioorg. Med. Chem. 14: 2162-2177 (2006); the PDB crystal structures PDB 2bgd, 2bge, 2cm7, 2cm8, 2cma, 2cmb, 2cmc described in “Structure-Based Design of Protein Tyrosine Phosphatase-1B Inhibitors”. Black, E. et al. Bioorg. Med. Chem. Lett. 15: 2503 (2005) and “Structural Basis for Inhibition of Protein-Tyrosine Phosphatase 1B by Isothiazolidinone Heterocyclic Phosphonate Mimetics.” Ala, P. J. et al. J. Biol. Chem. 281: 32784 (2006); the PDB crystal structures 2f6t and 2f6w described in “1,2,3,4-Tetrahydroisoquinolinyl sulfamic acids as phosphatase PTP1B inhibitors”. Klopfenstein, S. R. et al. Bioorg. Med Chem. Lett. 16: 1574-1578 (2006); the PDB crystal structures 2h4g, 2h4k, 2hb1 described in “Monocyclic thiophenes as protein tyrosine phosphatase 1B inhibitors: Capturing interactions with Asp48.” Wan, Z. K. et al. Bioorg. Med. Chem. Lett. 16: 4941-4945 (2006); the PDB crystal structures 2zn7 described in “Structure-based optimization of protein tyrosine phosphatase-1 B inhibitors: capturing interactions with arginine 24”. Wan, Z. K. et al. Chem Med Chem 3:1525-9 (2008); the PDB crystal structure 2nt7, 2nta described in “Probing acid replacements of thiophene PTP1B inhibitors.” Wan, Z. K. et al. Bioorg. Med. Chem. Lett. 17: 2913-2920 (2007); and, WO 2008148744 A1 assigned to Novartis AG titled “Thiadiazole derivatives as antidiabetic agents”. See also, the PDB crystal structures 1c84, 1c84, 1c85, 1c86, 1c88, 118g and described in “2-(oxalylamino)-benzoic acid is a general, competitive inhibitor of protein-tyrosine phosphatases”. Andersen, H. S. et al. J. Biol. Chem. 275: 7101-7108 (2000); “Structure-based design of a low molecular weight, nonphosphorus, nonpeptide, and highly selective inhibitor of protein-tyrosine phosphatase 1B.” Iversen, L. F. et al. J. Biol. Chem. 275: 10300-10307 (2000); and, “Steric hindrance as a basis for structure-based design of selective inhibitors of protein-tyrosine phosphatases”. Iversen, L. F. et al. Biochemistry 40: 14812-14820 (2001).

FIG. 7D provides non-limiting examples of Tyrosine-protein phosphatase non-receptor type 11, SHP2 Targeting Ligands wherein R represents exemplary points at which the spacer is attached. For additional examples and related ligands, see, the crystal structures PDB 4pvg and 305x and described in “Salicylic acid based small molecule inhibitor for the oncogenic Src homology-2 domain containing protein tyrosine phosphatase-2 (SHP2).” Zhang, X. et al. J. Med. Chem. 53: 2482-2493 (2010); and, the crystal structure PDB 5ehr and related ligands described in “Allosteric Inhibition of SHP2: Identification of a Potent, Selective, and Orally Efficacious Phosphatase Inhibitor.” Garcia Fortanet, J. et al. J. Med. Chem. 59: 7773-7782 (2016). Also, see the crystal structure PDB 5ehr described in “Allosteric Inhibition of SHP2: Identification of a Potent, Selective, and Orally Efficacious Phosphatase Inhibitor.” Garcia Fortanet, J. et al. J. Med. Chem. 59: 7773-7782 (2016) and “Allosteric inhibition of SHP2 phosphatase inhibits cancers driven by receptor tyrosine kinases.” Chen, Y. P. et al. Nature 535: 148-152 (2016).

FIG. 7E provides non-limiting examples of Tyrosine-protein phosphatase non-receptor type 22 Targeting Ligands wherein R represents exemplary points at which the spacer is attached. For additional examples and related ligands, see, the crystal structure PDB 4j51 described in “A Potent and Selective Small-Molecule Inhibitor for the Lymphoid-Specific Tyrosine Phosphatase (LYP), a Target Associated with Autoimmune Diseases.” He, Y. et al. J. Med. Chem. 56: 4990-5008 (2013).

FIG. 7F provides non-limiting examples of Scavenger mRNA-decapping enzyme DcpS Targeting Ligands wherein R represents exemplary points at which the spacer is attached. For additional examples and related ligands, see, the crystal structures PDB 3b17, 3b19, 3bla, 4qde, 4qdv, 4qeb and related ligands described in “DcpS as a therapeutic target for spinal muscular atrophy.” Singh, J. et al. ACS Chem. Biol. 3: 711-722 (2008).

FIG. 8A-8S provide non-limiting examples of BRD4 Bromodomain 1 Targeting Ligands wherein R represents exemplary points at which the spacer is attached. For additional examples and related ligands, see, the crystal structures PDB 3u5k and 3u51 and related ligands in Filippakopoulos, P. et al. “Benzodiazepines and benzotriazepines as protein interaction inhibitors targeting bromodomains of the BET family”, Bioorg. Med. Chem. 20: 1878-1886 (2012); the crystal structure PDB 3u51; the crystal structure PDB 3zyu and related ligands described in Dawson, M. A. et al. “Inhibition of Bet Recruitment to Chromatin as an Effective Treatment for Mll-Fusion Leukaemia.” Nature 478: 529 (2011); the crystal structure PDB 4bw1 and related ligands described in Mirguet, O. et al. “Naphthyridines as Novel Bet Family Bromodomain Inhibitors.” Chemmedchem 9: 589 (2014); the crystal structure PDB 4cf1 and related ligands described in Dittmann, A. et al. “The Commonly Used Pi3-Kinase Probe Ly294002 is an Inhibitor of Bet Bromodomains” ACS Chem. Biol. 9: 495 (2014); the crystal structure PDB 4e96 and related ligands described in Fish, P. V. et al. “Identification of a chemical probe for bromo and extra C-terminal bromodomain inhibition through optimization of a fragment-derived hit.” J. Med. Chem. 55: 9831-9837 (2012); the crystal structure PDB 4clb and related ligands described in Atkinson, S. J. et al. “The Structure Based Design of Dual Hdac/Bet Inhibitors as Novel Epigenetic Probes.” Medchemcomm 5: 342 (2014); the crystal structure PDB 4f3i and related ligands described in Zhang, G. et al. “Down-regulation of NF-{kappa}B Transcriptional Activity in HIV-associated Kidney Disease by BRD4 Inhibition.” J. Biol. Chem. 287: 28840-28851 (2012); the crystal structure PDB 4hxl and related ligands described in Zhao, L. “Fragment-Based Drug Discovery of 2-Thiazolidinones as Inhibitors of the Histone Reader BRD4 Bromodomain.” J. Med. Chem. 56: 3833-3851 (2013); the crystal structure PDB 4hxs and related ligands described in Zhao, L. et al. “Fragment-Based Drug Discovery of 2-Thiazolidinones as Inhibitors of the Histone Reader BRD4 Bromodomain.” J. Med. Chem. 56: 3833-3851 (2013); the crystal structure PDB 41rg and related ligands described in Gehling, V. S. et al. “Discovery, Design, and Optimization of Isoxazole Azepine BET Inhibitors.” ACS Med Chem Lett 4: 835-840 (2013); the crystal structure PDB 4mep and related ligands described in Vidler, L. R. “Discovery of Novel Small-Molecule Inhibitors of BRD4 Using Structure-Based Virtual Screening.” et al. J. Med. Chem. 56: 8073-8088 (2013); the crystal structures PDB 4nr8 and PDB 4c77 and related ligands described in Ember, S. W. et al. “Acetyl-lysine Binding Site of Bromodomain-Containing Protein 4 (BRD4) Interacts with Diverse Kinase Inhibitors”. ACS Chem. Biol. 9: 1160-1171 (2014); the crystal structure PDB 4o7a and related ligands described in Ember, S. W. et al. “Acetyl-lysine Binding Site of Bromodomain-Containing Protein 4 (BRD4) Interacts with Diverse Kinase Inhibitors.” ACS Chem. Biol. 9: 1160-1171 (2014); the crystal structure PDB 407b and related ligands described in “Acetyl-lysine Binding Site of Bromodomain-Containing Protein 4 (BRD4) Interacts with Diverse Kinase Inhibitors.” Ember, S. W. et al. (2014) ACS Chem. Biol. 9: 1160-1171; the crystal structure PDB 4o7c and related ligands described in Ember, S. W. et al. “Acetyl-lysine Binding Site of Bromodomain-Containing Protein 4 (BRD4) Interacts with Diverse Kinase Inhibitors”. ACS Chem. Biol. 9: 1160-1171 (2014); the crystal structure PDB 4gpj; the crystal structure PDB 4uix and related ligands described in Theodoulou, N. H. et al. “The Discovery of I-Brd9, a Selective Cell Active Chemical Probe for Bromodomain Containing Protein 9 Inhibition”. J. Med. Chem. 59: 1425 (2016); the crystal structure PDB 4uiz and related ligands described in Theodoulou, N. H., et al. “The Discovery of I-Brd9, a Selective Cell Active Chemical Probe for Bromodomain Containing Protein 9 Inhibition”. J. Med. Chem. 59: 1425 (2016); the crystal structure PDB 4wiv and related ligands described in McKeown, M. R. et al. “Biased multicomponent reactions to develop novel bromodomain inhibitors.” J. Med. Chem. 57: 9019-9027 (2014); the crystal structure PDB 4x2i and related ligands described in Taylor, A. M. et al. “Discovery of Benzotriazolo[4,3-d][1,4]diazepines as Orally Active Inhibitors of BET Bromodomains.” ACS Med. Chem. Lett. 7: 145-150 (2016); the crystal structure PDB 4yh3; And related ligands described in Duffy, B. C. “Discovery of a new chemical series of BRD4(1) inhibitors using protein-ligand docking and structure-guided design.” Bioorg. Med. Chem. Lett. 25: 2818-2823 (2015); the crystal structure PDB 4yh4 and related ligands described in Duffy, B. C. “Discovery of a new chemical series of BRD4(1) inhibitors using protein-ligand docking and structure-guided design.” Bioorg. Med. Chem. Lett. 25: 2818-2823 (2015); the crystal structure PDB 4z1q and related ligands described in Taylor, A. M. “Discovery of Benzotriazolo[4,3-d][1,4]diazepines as Orally Active Inhibitors of BET Bromodomains.” ACS Med Chem. Lett. 7: 145-150 (2016); the crystal structure PDB 4zw1; the crystal structure PDB 5a5s and related ligands described in Demont, E. H. “Fragment-Based Discovery of Low-Micromolar Atad2 Bromodomain Inhibitors. J. Med. Chem. 58: 5649 (2015); the crystal structure PDB 5a85 and related ligands described in Bamborough, P. “Structure-Based Optimization of Naphthyridones Into Potent Atad2 Bromodomain Inhibitors” J. Med. Chem. 58: 6151 (2015); the crystal structure PDB 5acy and related ligands described in Sullivan, J. M. “Autism-Like Syndrome is Induced by Pharmacological Suppression of Bet Proteins in Young Mice.” J. Exp. Med 212: 1771 (2015); the crystal structure PDB 5ad2 and related ligands described in Waring, M. J. et al. “Potent and Selective Bivalent Inhibitors of Bet Bromodomains”. Nat. Chem. Biol. 12: 1097 (2016); the crystal structure PDB 5cfw and related ligands described in Chekler, E. L. et al. “Transcriptional Profiling of a Selective CREB Binding Protein Bromodomain Inhibitor Highlights Therapeutic Opportunities.” Chem. Biol. 22: 1588-1596 (2015); the crystal structure PDB 5cqt and related ligands described in Xue, X. et al. “Discovery of Benzo[cd]indol-2(1H)-ones as Potent and Specific BET Bromodomain Inhibitors: Structure-Based Virtual Screening, Optimization, and Biological Evaluation”. J. Med. Chem. 59: 1565-1579 (2016); the crystal structure PDB 5d3r and related ligands described in Hugle, M. et al. “4-Acyl Pyrrole Derivatives Yield Novel Vectors for Designing Inhibitors of the Acetyl-Lysine Recognition Site of BRD4(1)”. J. Med. Chem. 59: 1518-1530 (2016); the crystal structure PDB 5dlx and related ligands described in Milhas, S. et al. “Protein-Protein Interaction Inhibition (2P2I)-Oriented Chemical Library Accelerates Hit Discovery.” (2016) ACS Chem. Biol. 11: 2140-2148; the crystal structure PDB 5dlz and related ligands described in Milhas, S. et al. “Protein-Protein Interaction Inhibition (2P2I)-Oriented Chemical Library Accelerates Hit Discovery.” ACS Chem. Biol. 11: 2140-2148 (2016); the crystal structure PDB 5dw2 and related ligands described in Kharenko, O. A. et al. “RVX-297—a novel BD2 selective inhibitor of BET bromodomains.” Biochem. Biophys. Res. Commun. 477: 62-67 (2016); the crystal structure PDB 5dlx; the crystal structure PDB 5his and related ligands described in Albrecht, B. K. et al. “Identification of a Benzoisoxazoloazepine Inhibitor (CPI-0610) of the Bromodomain and Extra-Terminal (BET) Family as a Candidate for Human Clinical Trials.” J. Med. Chem. 59: 1330-1339 (2016); the crystal structure PDB 5ku3 and related ligands described in Crawford, T. D. et al. “Discovery of a Potent and Selective in Vivo Probe (GNE-272) for the Bromodomains of CBP/EP300”. J. Med. Chem. 59: 10549-10563 (2016); the crystal structure PDB 51j2 and related ligands described in Bamborough, P. et al. “A Chemical Probe for the ATAD2 Bromodomain.” Angew. Chem. Int. Ed Engl. 55: 11382-11386 (2016); the crystal structure PDB 5dlx and related ligands described in Wang, L. “Fragment-based, structure-enabled discovery of novel pyridones and pyridone macrocycles as potent bromodomain and extra-terminal domain (BET) family bromodomain inhibitors”. J. Med. Chem. 10.1021/acs. jmedchem.7b00017 (2017); WO 2015169962 A1 titled “Benzimidazole derivatives as BRD4 inhibitors and their preparation and use for the treatment of cancer” assigned to Boehringer Ingelheim International GmbH, Germany; and, WO 2011143669 A2 titled “Azolodiazepine derivatives and their preparation, compositions and methods for treating neoplasia, inflammatory disease and other disorders” assigned to Dana-Farber Cancer Institute, Inc, USA.

FIG. 8T-8V provide non-limiting examples of ALK Targeting Ligands wherein R represents exemplary points at which the spacer is attached. For additional examples and related ligands, see, the crystal structures PDB 2xb7 and 2xba and related ligands described in Bossi, R. T. et al. “Crystal Structures of Anaplastic Lymphoma Kinase in Complex with ATP Competitive Inhibitors” Biochemistry 49: 6813-6825 (2010); the crystal structures PDB 2yfx, 4ccb, 4ccu, and 4cd0 and related ligands described in Huang, Q. et al. “Design of Potent and Selective Inhibitors to Overcome Clinical Anaplastic Lymphoma Kinase Mutations Resistant to Crizotinib.” J Med Chem. 57: 1170 (2014); the crystal structures PDB, 4cli, 4cmo, and 4cnh and related ligands described in Johnson, T. W. et al. “Discovery of (10R)-7-Amino-12-Fluoro-2,10,16-Trimethyl-15-Oxo-10,15,16,17-Tetrahydro-2H-8,4-(Metheno)Pyrazolo[4,3-H][2,5,11]Benzoxadiazacyclotetradecine-3-Carbonitrile (Pf-06463922), a Macrocyclic Inhibitor of Alk/Ros1 with Pre-Clinical Brain Exposure and Broad Spectrum Potency Against Alk-Resistant Mutations.” J. Med. Chem. 57: 4720 (2014); the crystal structure PDB 4fny and related ligands described in Epstein, L. F. et al. “The R1275Q Neuroblastoma Mutant and Certain ATP-competitive Inhibitors Stabilize Alternative Activation Loop Conformations of Anaplastic Lymphoma Kinase.” J. Biol. Chem. 287: 37447-37457 (2012). the crystal structure PDB 4dce and related ligands described in Bryan, M. C. et al “Rapid development of piperidine carboxamides as potent and selective anaplastic lymphoma kinase inhibitors.” J. Med. Chem. 55: 1698-1705 (2012); the crystal structure PDB 4joa and related ligands described in Gummadi, V. R. et al. “Discovery of 7-azaindole based anaplastic lymphoma kinase (ALK) inhibitors: wild type and mutant (L1196M) active compounds with unique binding mode.” (2013) Bioorg. Med Chem. Lett. 23: 4911-4918; and, the crystal structure PDB 5iui and related ligands described in Tu, C. H. et al. “Pyrazolylamine Derivatives Reveal the Conformational Switching between Type I and Type II Binding Modes of Anaplastic Lymphoma Kinase (ALK).” J. Med. Chem. 59: 3906-3919 (2016).

FIG. 8W-8X provide non-limiting examples of BTK Targeting Ligands wherein R represents exemplary points at which the spacer is attached. For additional examples and related ligands, see, the crystal structure PDB 3gen, 3piz and related ligands described in Marcotte, D. J. et al. “Structures of human Bruton's tyrosine kinase in active and inactive conformations suggest a mechanism of activation for TEC family kinases.” Protein Sci. 19: 429-439 (2010) and Kuglstatter, A. et al. “Insights into the conformational flexibility of Bruton's tyrosine kinase from multiple ligand complex structures” Protein Sci. 20: 428-436” (2011); the crystal structure PDB 3ocs, 4ot6 and related ligands described in Lou, Y. et al. “Structure-Based Drug Design of RN486, a Potent and Selective Bruton's Tyrosine Kinase (BTK) Inhibitor, for the Treatment of Rheumatoid Arthritis” J. Med. Chem. 58: 512-516 (2015); the crystal structures PDB 5fbn and 5fbo and related ligands described in Liu, J. et al. “Discovery of 8-Amino-imidazo[1,5-a]pyrazines as Reversible BTK Inhibitors for the Treatment of Rheumatoid Arthritis.” ACS Med. Chem. Lett. 7: 198-203 (2016); the crystal structure PDB 3pix and related ligands described in Kuglstatter, A. et al. “Insights into the conformational flexibility of Bruton's tyrosine kinase from multiple ligand complex structures.” Protein Sci. 20: 428-436 (2011); and, the crystal structure PDB 3pij and related ligands described in Bujacz, A. et al. “Crystal structures of the apo form of beta-fructofuranosidase from Bifidobacterium longum and its complex with fructose.” Febs J. 278: 1728-1744 (2011).

FIG. 8Y provides non-limiting examples of FLT3 Targeting Ligands wherein R represents exemplary points at which the spacer is attached. For additional examples and related ligands, see, the crystal structures PDB 4xuf and 4rt7 and related ligands described in Zorn, J. A. et al. “Crystal Structure of the FLT3 Kinase Domain Bound to the Inhibitor Quizartinib (AC220)”. Plos One 10: e0121177-e0121177 (2015).

FIG. 8Z-8AA provide non-limiting examples of TNIK Targeting Ligands wherein R represents exemplary points at which the spacer is attached. For additional examples and related ligands, see, the crystal structure PDB 2x7f; the crystal structures PDB 5ax9 and 5d7a; and, related ligands described in Masuda, M. et al. “TNIK inhibition abrogates colorectal cancer stemness.” Nat Commun 7: 12586-12586 (2016).

FIG. 8BB-8CC provide non-limiting examples of NTRK1, NTRK2, and NTRK3 Targeting Ligands wherein R represents exemplary points at which the spacer is attached. For additional examples and related ligands, see, the crystal structure PDB 4aoj and related ligands described in Wang, T. et al. “Discovery of Disubstituted Imidazo[4,5-B]Pyridines and Purines as Potent Trka Inhibitors.” ACS Med. Chem. Lett. 3: 705 (2012); the crystal structures PDB 4pmm, 4pmp, 4pms and 4pmt and related ligands described in Stachel, S. J. et al. “Maximizing diversity from a kinase screen: identification of novel and selective pan-Trk inhibitors for chronic pain.” J. Med. Chem. 57: 5800-5816 (2014); the crystal structures PDB 4yps and 4yne and related ligands described in Choi, H. S. et al. “(R)-2-Phenylpyrrolidine Substituted Imidazopyridazines: A New Class of Potent and Selective Pan-TRK Inhibitors.” ACS Med. Chem. Lett. 6: 562-567 (2015); the crystal structures PDB 4at5 and 4at3 and related ligands described in Bertrand, T. et al. “The Crystal Structures of Trka and Trkb Suggest Key Regions for Achieving Selective Inhibition.” J. Mol. Biol. 423: 439 (2012); and, the crystal structures PDB 3v5q and 4ymj and related ligands described in Albaugh, P. et al. “Discovery of GNF-5837, a selective TRK Inhibitor with efficacy in rodent cancer tumor models.” ACS Med. Chem. Lett. 3: 140-145 (2012) and Choi, H. S. et al. “(R)-2-Phenylpyrrolidine Substitute Imidazopyridazines: a New Class of Potent and Selective Pan-TRK Inhibitors.” ACS Med Chem Lett 6: 562-567 (2015).

FIG. 8DD-8EE provide non-limiting examples of FGFR1 Targeting Ligands wherein R represents exemplary points at which the spacer is attached. For additional examples and related ligands, see, the crystal structures PDB 3tto and 2fgi and related ligands described in Brison, Y. et al. “Functional and structural characterization of alpha-(1-2) branching sucrase derived from DSR-E glucansucrase.” J. Biol. Chem. 287: 7915-7924 (2012) and Mohammadi, M. et al. “Crystal structure of an angiogenesis inhibitor bound to the FGF receptor tyrosine kinase domain.” EMBO J. 17: 5896-5904 (1998); the crystal structure PDB 4fb3; the crystal structure PDB 4rwk and related ligands described in Harrison, C. et al. “Polyomavirus large T antigen binds symmetrical repeats at the viral origin in an asymmetrical manner.” J. Virol. 87: 13751-13759 (2013); the crystal structure PDB 4rwl and related ligands described in Sohl, C. D. et al. “Illuminating the Molecular Mechanisms of Tyrosine Kinase Inhibitor Resistance for the FGFR1 Gatekeeper Mutation: The Achilles' Heel of Targeted Therapy.” ACS Chem. Biol. 10: 1319-1329 (2015); the crystal structure PDB 4uwc; the crystal structure PDB 4v01 and related ligands described in Tucker, J. A. et al. “Structural Insights Into Fgfr Kinase Isoform Selectivity: Diverse Binding Modes of Azd4547 and Ponatinib in Complex with Fgfr1 and Fgfr4.” Structure 22: 1764 (2014).; the crystal structure PDB 5a46 and related ligands described in Klein, T. et al. “Structural and Dynamic Insights Into the Energetics of Activation Loop Rearrangement in Fgfr1 Kinase.” Nat. Commun. 6: 7877 (2015); and, the crystal structure PDB 5ew8 and related ligands described in Patani, H. et al. “Landscape of activating cancer mutations in FGFR kinases and their differential responses to inhibitors in clinical use.” Oncotarget 7: 24252-24268 (2016).

FIG. 8FF provides non-limiting examples of FGFR2 and FGFR3 Targeting Ligands wherein R represents exemplary points at which the spacer is attached. For additional examples and related ligands, see, the crystal structure PDB 2pvf and related ligands described in Chen, H. et al. “A molecular brake in the kinase hinge region regulates the activity of receptor tyrosine kinases.” Mol. Cell 27: 717-730 (2007); and “Structure-based drug design of 1,3,5-triazine and pyrimidine derivatives as novel FGFR3 inhibitors with high selectivity over VEGFR2” Bioorg Med Chem 2020, 28, 115453.

FIG. 8GG provides non-limiting examples of FGFR4 Targeting Ligands wherein R represents exemplary points at which the spacer is attached. For additional examples and related ligands, see, the crystal structure PDB 4tyi and related ligands described in Lesca, E. et al. “Structural analysis of the human fibroblast growth factor receptor 4 kinase.” J. Mol. Biol. 426: 3744-3756 (2014).

FIG. 8HH-8II provide non-limiting examples of MET Targeting Ligands wherein R represents exemplary points at which the spacer is attached. For additional examples and related ligands, see, the crystal structures PDB 3qti and 3zcl; the crystal structures PDB 4xmo, 4xyf, and 3zcl and related ligands described in Peterson, E. A. et al. “Discovery of Potent and Selective 8-Fluorotriazolopyridine c-Met Inhibitors.” J. Med. Chem. 58: 2417-2430 (2015) and Cui, J. J. et al. “Lessons from (S)-6-(1-(6-(1-Methyl-1H-Pyrazol-4-Yl)-[1,2, 4]Triazolo[4,3-B]Pyridazin-3-Yl)Ethyl)Quinoline (Pf-04254644), an Inhibitor of Receptor Tyrosine Kinase C-met with High Protein Kinase Selectivity But Broad Phosphodiesterase Family Inhibition Leading to Myocardial Degeneration in Rats.” J. Med. Chem. 56: 6651 (2013); the crystal structure PDB 5eyd and related ligands described in Boezio, A. A. et al. “Discovery of (R)-6-(1-(8-Fluoro-6-(1-methyl-1H-pyrazol-4-yl)-[1,2,4]triazolo[4,3-a]pyridin-3-yl)ethyl)-3-(2-methoxyethoxy)-1,6-naphthyridin-5(6H)-one (AMG 337), a Potent and Selective Inhibitor of MET with High Unbound Target Coverage and Robust In Vivo Antitumor Activity.” J. Med. Chem. 59: 2328-2342 (2016); the crystal structure PDB 3ce3 and related ligands described in Kim, K. S. et al. “Discovery of pyrrolopyridine-pyridone based inhibitors of Met kinase: synthesis, X-ray crystallographic analysis, and biological activities.” J. Med. Chem. 51: 5330-5341 (2008); the crystal structure PDB 2rfn and related ligands described in Bellon, S. F. et al. “c-Met inhibitors with novel binding mode show activity against several hereditary papillary renal cell carcinoma-related mutations.” J. Biol. Chem. 283: 2675-2683 (2008); and, the crystal structure PDB 5dg5 and related ligands described in Smith, B. D. et al “Altiratinib Inhibits Tumor Growth, Invasion, Angiogenesis, and Microenvironment-Mediated Drug Resistance via Balanced Inhibition of MET, TIE2, and VEGFR2.”. Mol. Cancer Ther. 14: 2023-2034 (2015).

FIG. 8JJ provides non-limiting examples of JAK1 Targeting Ligands wherein R represents exemplary points at which the spacer is attached. For additional examples and related ligands, see, the crystal structure PDB 4ivd and related ligands described in Zak, M. et al. “Identification of C-2 Hydroxyethyl Imidazopyrrolopyridines as Potent JAK1 Inhibitors with Favorable Physicochemical Properties and High Selectivity over JAK2.” J. Med. Chem. 56: 4764-4785 (2013); the crystal structure PDB 5ele and related ligands described in Vasbinder, M. M. et al. “Identification of azabenzimidazoles as potent JAK1 selective inhibitors.” Bioorg. Med Chem. Lett. 26: 60-67 (2016); the crystal structure PDB 5hx8 and related ligands described in Simov, V., et al. “Structure-based design and development of (benz)imidazole pyridones as JAK1-selective kinase inhibitors.” Bioorg. Med. Chem. Lett. 26: 1803-1808 (2016); the crystal structure PDB 5hx8 and related ligands described in Caspers, N. L. et al. “Development of a high-throughput crystal structure-determination platform for JAK1 using a novel metal-chelator soaking system”. Acta Crystallogr. Sect. F72: 840-845 (2016); and, Kettle, J. G. “Discovery of the JAK1 selective kinase inhibitor AZD4205”, AACR National Meeting, April 2017.

FIG. 8KK-8LL provide non-limiting examples of JAK2 Targeting Ligands wherein R represents exemplary points at which the spacer is attached. For additional examples and related ligands, see, the crystal structure PDB 3ugc and related ligands described in Andraos, R. et al. “Modulation of activation-loop phosphorylation by JAK inhibitors is binding mode dependent.” Cancer Discov 2: 512-523 (2012); the crystal structures PDB 5cf4, 5cf5, 5cf6 and 5cf8 and related ligands described in Hart, A. C. et al. “Structure-Based Design of Selective Janus Kinase 2 Imidazo[4,5-d]pyrrolo[2,3-b]pyridine Inhibitors.” ACS Med. Chem. Lett. 6: 845-849 (2015); the crystal structure PDB 5aep and related ligands described in Brasca, M. G. et al “Novel Pyrrole Carboxamide Inhibitors of Jak2 as Potential Treatment of Myeloproliferative Disorders” Bioorg. Med. Chem. 23: 2387 (2015); the crystal structures PDB 4ytf, 4yth and 4yti and related ligands described in Farmer, L. J. et al. “Discovery of VX-509 (Decernotinib): A Potent and Selective Janus Kinase 3 Inhibitor for the Treatment of Autoimmune Diseases.” J. Med. Chem. 58: 7195-7216 (2015); the crystal structure PDB 4ytf, 4yth, 4yti and related ligands described in Menet, C. J. et al. “Triazolopyridines as Selective JAK1 Inhibitors: From Hit Identification to GLPG0634.” J. Med. Chem. 57: 9323-9342 (2014); the crystal structure PDB 4ji9 and related ligands described in Siu, M. et al. “2-Amino-[1,2,4]triazolo[1,5-a]pyridines as JAK2 inhibitors.” Bioorg. Med Chem. Lett. 23: 5014-5021 (2013); and, the crystal structures PDB 3io7 and 3iok and related ligands described in Schenkel, L. B. et al. “Discovery of potent and highly selective thienopyridine janus kinase 2 inhibitors.” J. Med. Chem. 54: 8440-8450 (2011).

FIG. 8MM provides non-limiting examples of JAK3 Targeting Ligands wherein R represents exemplary points at which the spacer is attached. For additional examples and related ligands, see, the crystal structure PDB 3zc6 and related ligands described in Lynch, S. M. et al. “Strategic Use of Conformational Bias and Structure Based Design to Identify Potent Jak3 Inhibitors with Improved Selectivity Against the Jak Family and the Kinome.” Bioorg. Med Chem. Lett. 23: 2793 (2013); and, the crystal structures PDB 4hvd, 4i6q, and 3zep and related ligands described in Soth, M. et al. “3-Amido Pyrrolopyrazine JAK Kinase Inhibitors: Development of a JAK3 vs JAK1 Selective Inhibitor and Evaluation in Cellular and in Vivo Models.” J. Med. Chem. 56: 345-356 (2013) and Jaime-Figueroa, S. et al. “Discovery of a series of novel 5H-pyrrolo[2,3-b]pyrazine-2-phenyl ethers, as potent JAK3 kinase inhibitors.” Bioorg. Med. Chem. Lett. 23: 2522-2526 (2013).

FIG. 8NN-8OO provide non-limiting examples of KIT Targeting Ligands wherein R represents exemplary points at which the spacer is attached. For additional examples and related ligands, see, the crystal structure PDB 1t46 and related ligands described in Mol, C. D. et al. “Structural basis for the autoinhibition and STI-571 inhibition of c-Kit tyrosine kinase.” J Biol. Chem. 279: 31655-31663 (2004); and, the crystal structure PDB 4u0i and related ligands described in Garner, A. P. et al. “Ponatinib Inhibits Polyclonal Drug-Resistant KIT Oncoproteins and Shows Therapeutic Potential in Heavily Pretreated Gastrointestinal Stromal Tumor (GIST) Patients.” Clin. Cancer Res. 20: 5745-5755 (2014).

FIG. 88PP-8VV provide non-limiting examples of EGFR Targeting Ligands wherein R represents exemplary points at which the spacer is attached. For additional examples and related ligands, see, the crystal structures PDB 5hcy, 4rj4, and 5cav; Heald, R., “Noncovalent Mutant Selective Epidermal Growth Factor Receptor Inhibitors: A Lead Optimization Case Study”, J. Med. Chem. 58, 8877-8895 (2015); Hanano, E. J., “Discovery of Selective and Noncovalent Diaminopyrimidine-Based Inhibitors of Epidermal Growth Factor Receptor Containing the T790M Resistance Mutation.” J. Med. Chem., 57, 10176-10191 (2014); Chan, B. K. et al. “Discovery of a Noncovalent, Mutant-Selective Epidermal Growth Factor Receptor Inhibitor” J. Med. Chem. 59, 9080 (2016); the crystal structure PDB 5d41 and related ligands described in Jia, Y. et al., “Overcoming EGFR(T790M) and EGFR(C797S) resistance with mutant-selective allosteric inhibitors” Nature 534, 129 (2016); Ward, R. A. “Structure- and reactivity-based development of covalent inhibitors of the activating and gatekeeper mutant forms of the epidermal growth factor receptor (EGFR)” J. Med. Chem. 56, 7025-7048 (2013); the crystal structure PDB 4zau and related ligands described in “Discovery of a Potent and Selective EGFR Inhibitor (AZD9291) of Both Sensitizing and T790M Resistance Mutations That Spares the Wild Type Form of the Receptor” J. Med. Chem., 57(20), 8249-8267 (2014); the crystal structure PDB 5em7 and related ligands described in Bryan, M. C. et al. “Pyridones as Highly Selective, Noncovalent Inhibitors of T790M Double Mutants of EGFR” ACS Med. Chem. Lett., 7 (1), 100-104 (2016); the crystal structure PDB 3IKA and related ligands described in Zhou, W. et al. “Novel mutant-selective EGFR kinase inhibitors against EGFR T790M” Nature 462(7276), 1070-1074 (2009); the crystal structure see PDB 5feq and related ligands described in Lelais, G., J. “Discovery of (R,E)-N-(7-Chloro-1-(1-[4-(dimethylamino)but-2-enoyl]azepan-3-yl)-1H-benzo[d]imidazol-2-yl)-2-methylisonicotinamide (EGF816), a Novel, Potent, and WT Sparing Covalent Inhibitor of Oncogenic (L858R, ex19del) and Resistant (T790M) EGFR Mutants for the Treatment of EGFR Mutant Non-Small-Cell Lung Cancers” Med. Chem., 59 (14), 6671-6689 (2016); Lee, H.-J. “Noncovalent Wild-type-Sparing Inhibitors of EGFR T790M” Cancer Discov. 3(2): 168-181 (2013); the crystal structure PDB 5j7h and related ligands described in Huang, W-S. et al. “Discovery of Brigatinib (AP26113), a Phosphine Oxide-Containing, Potent, Orally Active Inhibitor of Anaplastic Lymphoma Kinase.” J. Med. Chem. 59: 4948-4964 (2016); the crystal structure PDB 4v0g and related ligands described in Hennessy, E. J. et al. “Utilization of Structure-Based Design to Identify Novel, Irreversible Inhibitors of EGFR Harboring the T790M Mutation.” ACS. Med. Chem. Lett. 7: 514-519 (2016); the crystal structure PDB 5hg7 and related ligands described in Cheng, H. “Discovery of 1-{(3R,4R)-3-[({5-Chloro-2-[(1-methyl-1H-pyrazol-4-yl)amino]-7H-pyrrolo[2,3-d]pyrimidin-4-yl}oxy)methyl]-4-methoxypyrrolidin-1-yl}prop-2-en-1-one (PF-06459988), a Potent, WT Sparing, Irreversible Inhibitor of T790M-Containing EGFR Mutants.” J. Med. Chem. 59: 2005-2024 (2016); Hao, Y. “Discovery and Structural Optimization of N5-Substituted 6,7-Dioxo-6,7-dihydropteridines as Potent and Selective Epidermal Growth Factor Receptor (EGFR) Inhibitors against L858R/T790M Resistance Mutation.” J. Med. Chem. 59: 7111-7124 (2016); the crystal structure PDB 5ug8, 5ug9, and Sugc and related ligands described in Planken, S. “Discovery of N-((3R,4R)-4-Fluoro-1-(6-((3-methoxy-1-methyl-1H-pyrazol-4-yl)amino)-9-methyl-9H-purin-2-yl)pyrrolidine-3-yl)acrylamide (PF-06747775) through Structure-Based Drug Design: A High Affinity Irreversible Inhibitor Targeting Oncogenic EGFR Mutants with Selectivity over Wild-Type EGFR.” J. Med. Chem. 60: 3002-3019 (2017); the crystal structure PDB 5gnk and related ligands described in Wang, A. “Discovery of (R)-1-(3-(4-Amino-3-(3-chloro-4-(pyridin-2-ylmethoxy)phenyl)-1H-pyrazolo[3,4-d]pyrimidin-1-yl)piperidin-1-yl)prop-2-en-1-one (CHMFL-EGFR-202) as a Novel Irreversible EGFR Mutant Kinase Inhibitor with a Distinct Binding Mode.” J. Med. Chem. 60: 2944-2962 (2017); and, Juchum, M. “Trisubstituted imidazoles with a rigidized hinge binding motif act as single digit nM inhibitors of clinically relevant EGFR L858R/T790M and L858R/T790M/C797S mutants: An example of target hopping.” J. Med Chem. DOI: 10.1021/acs. jmedchem.7b00178 (2017).

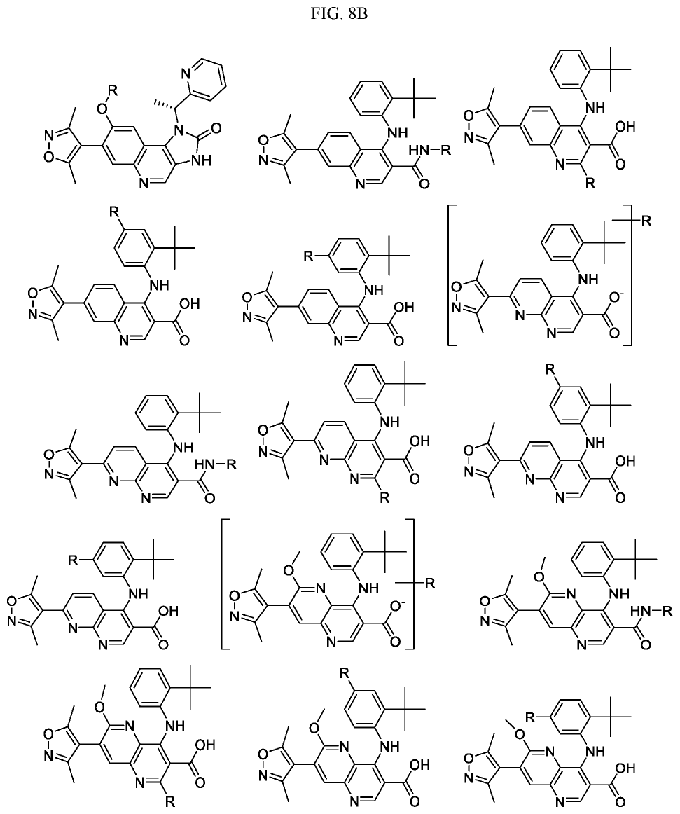

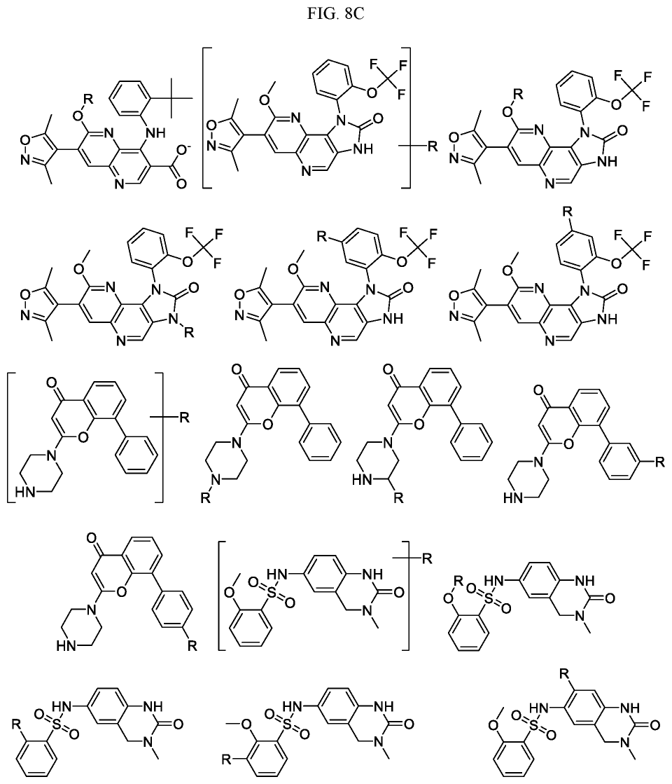

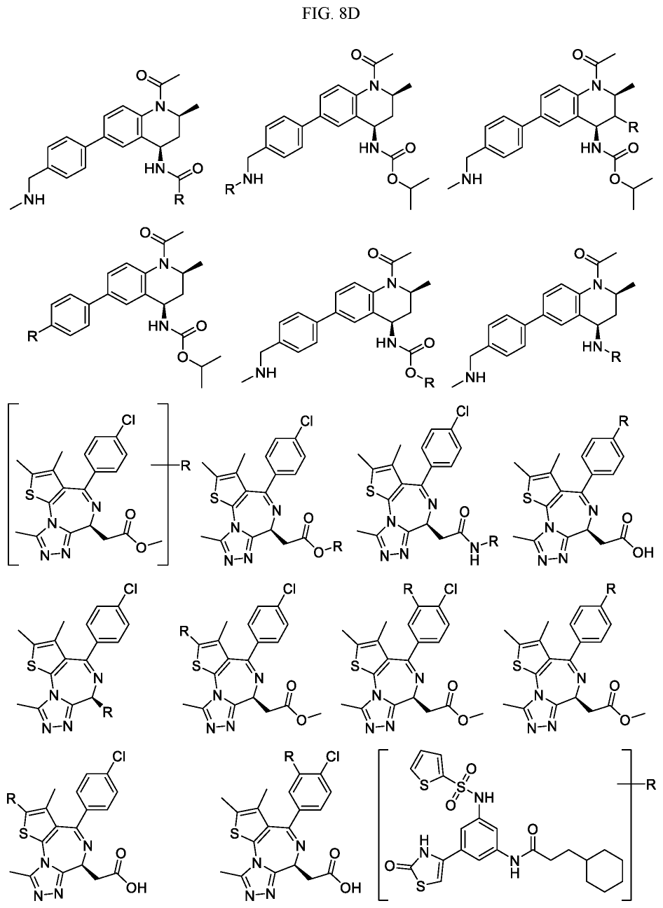

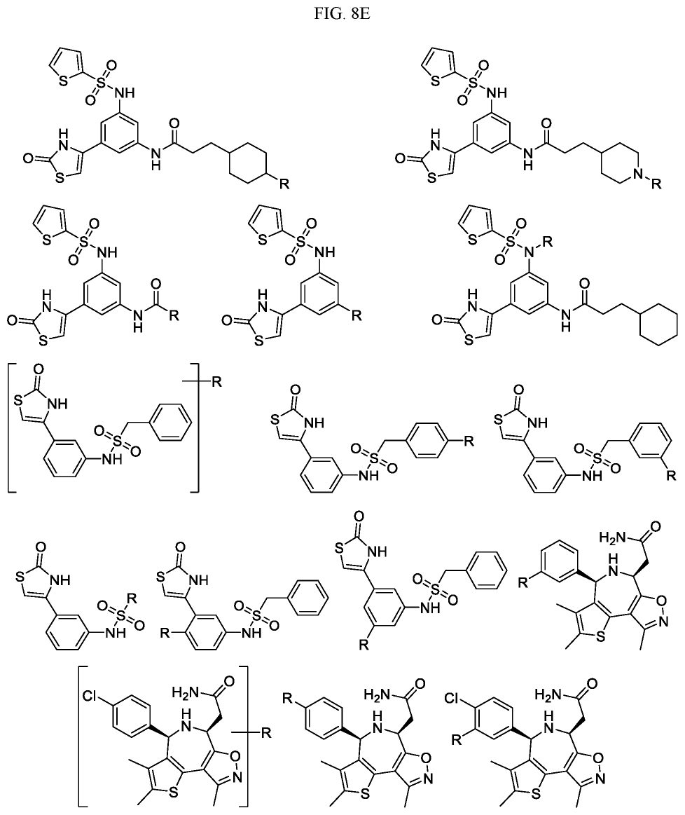

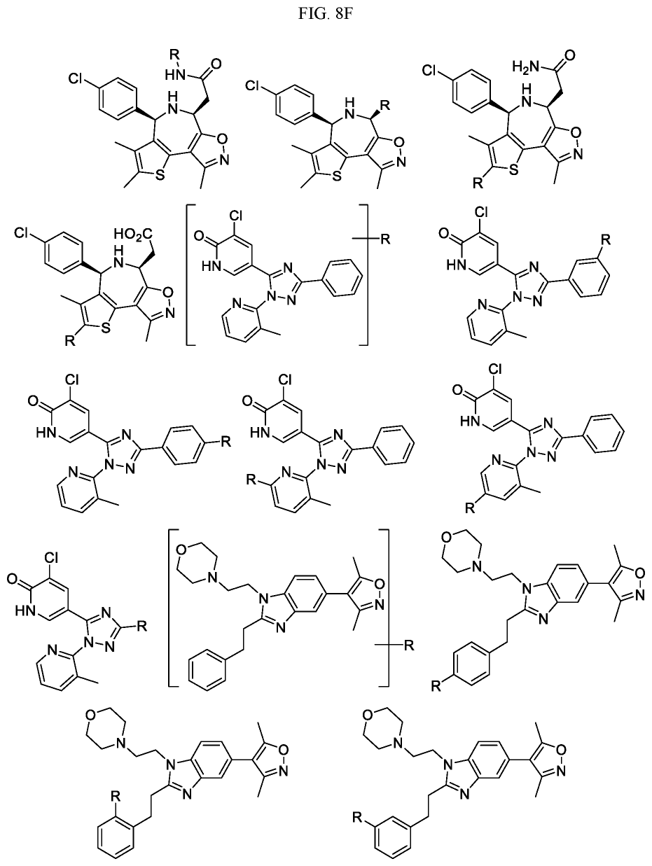

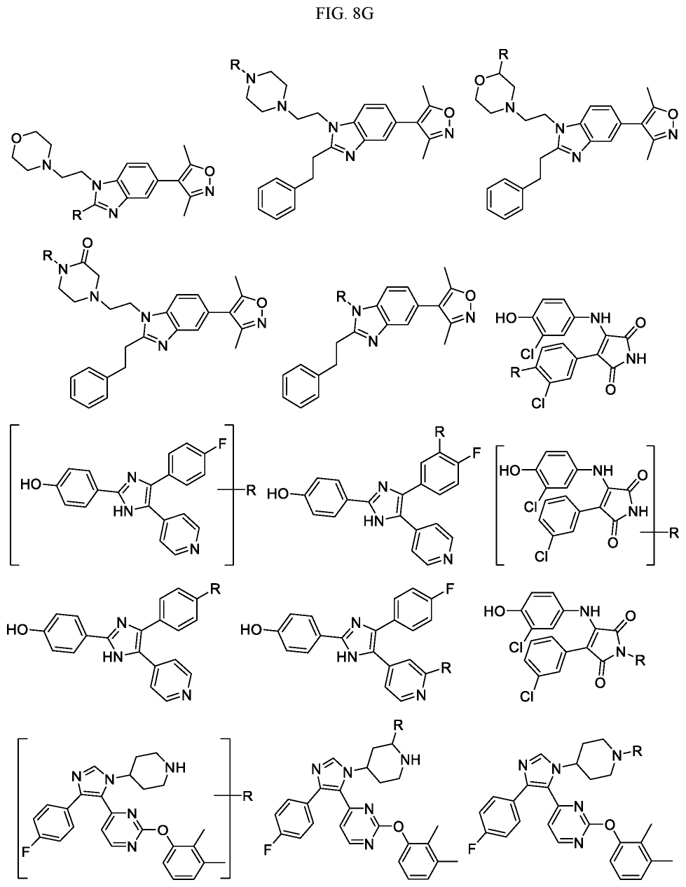

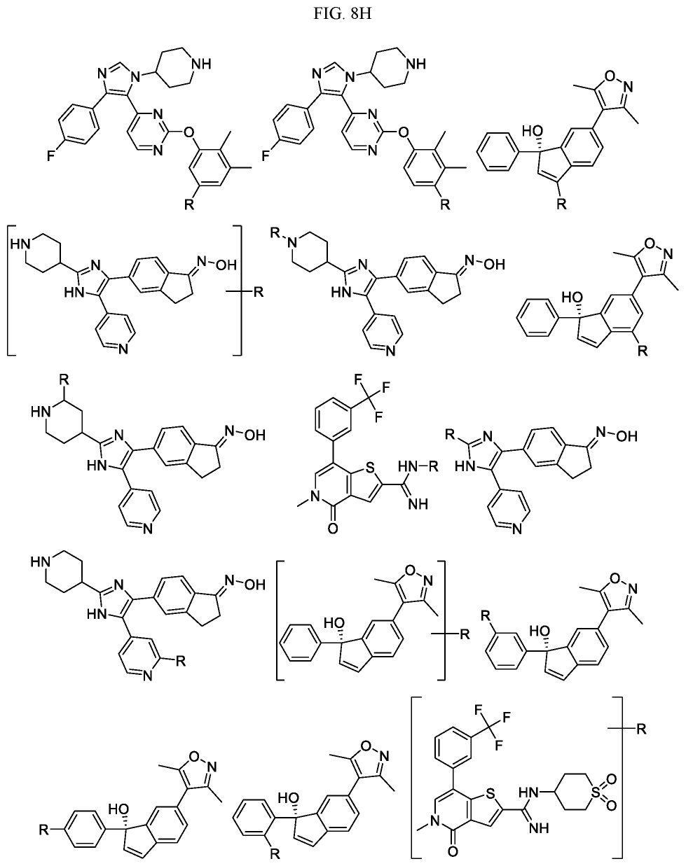

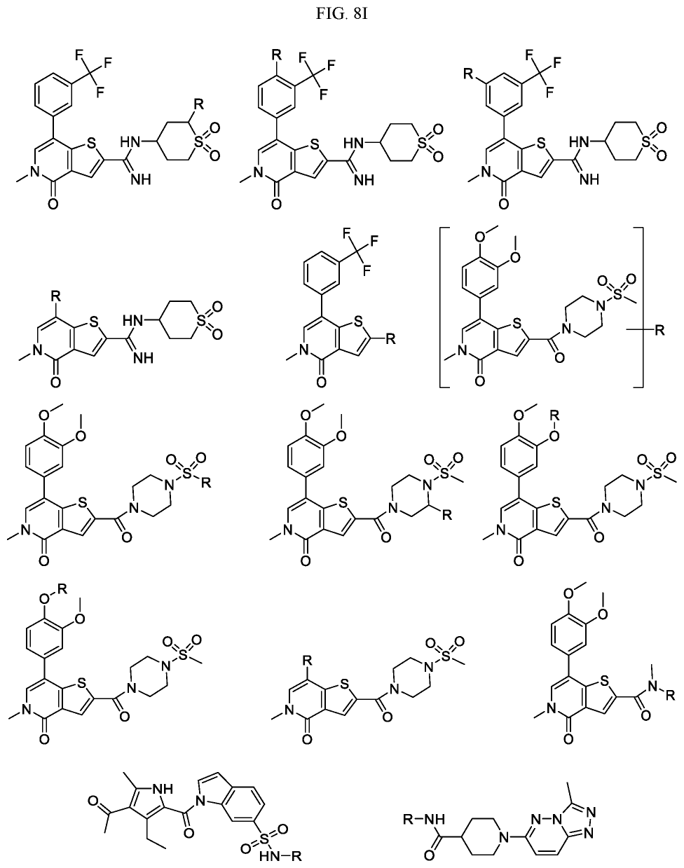

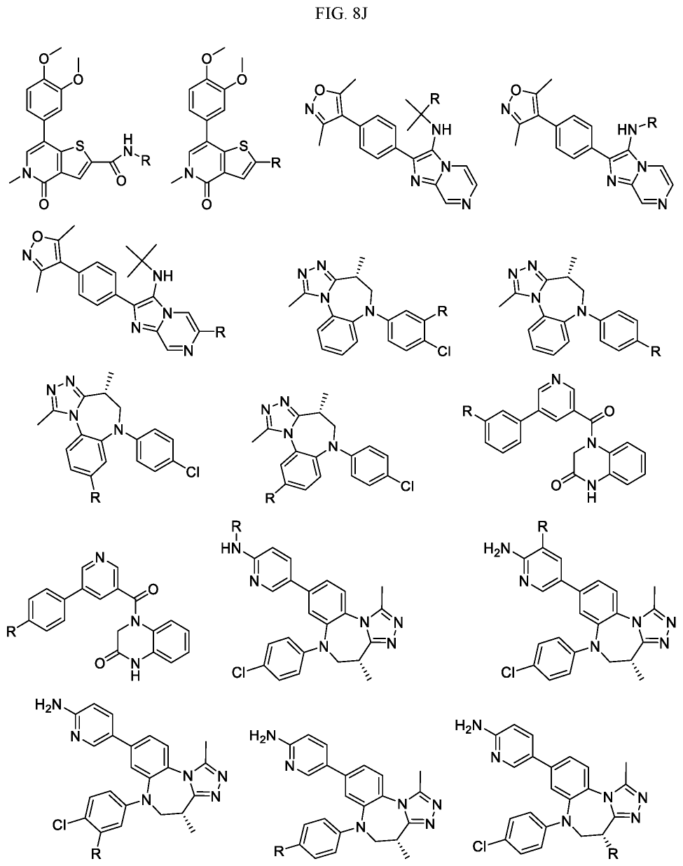

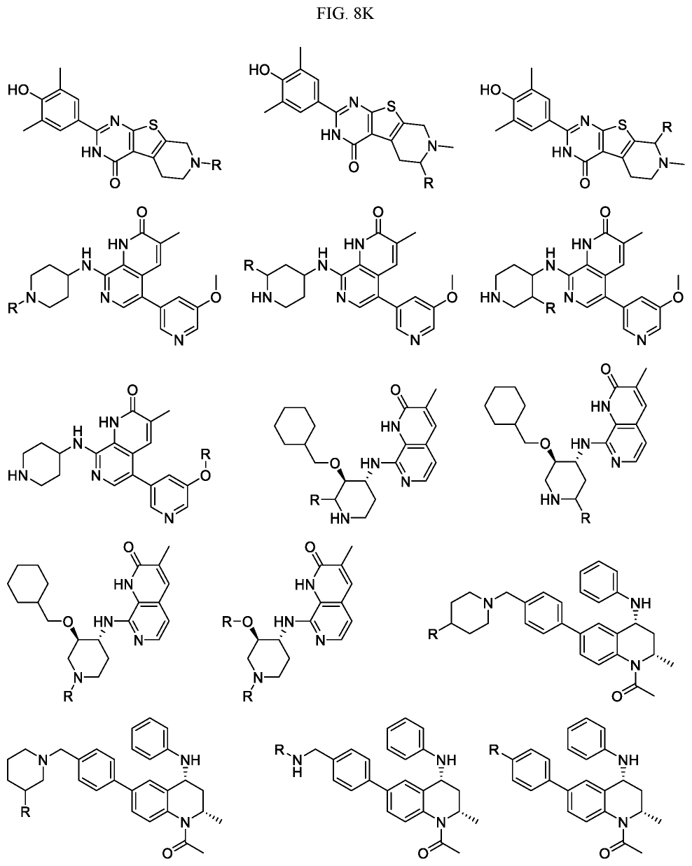

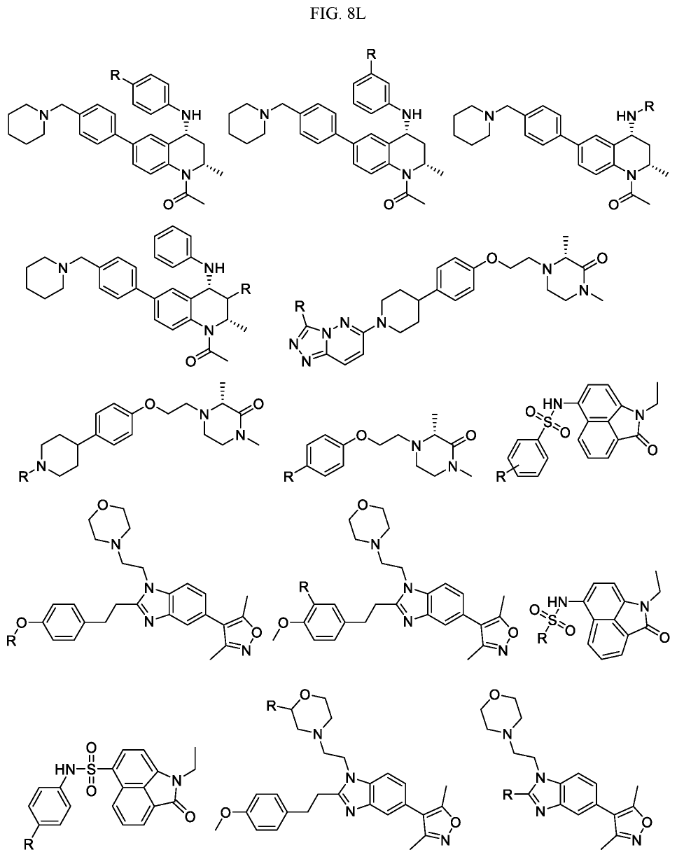

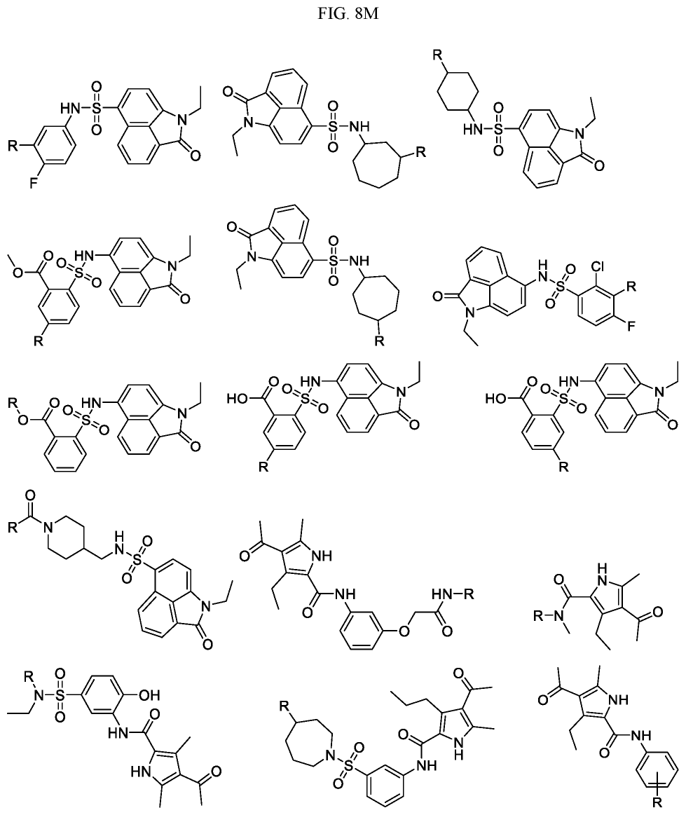

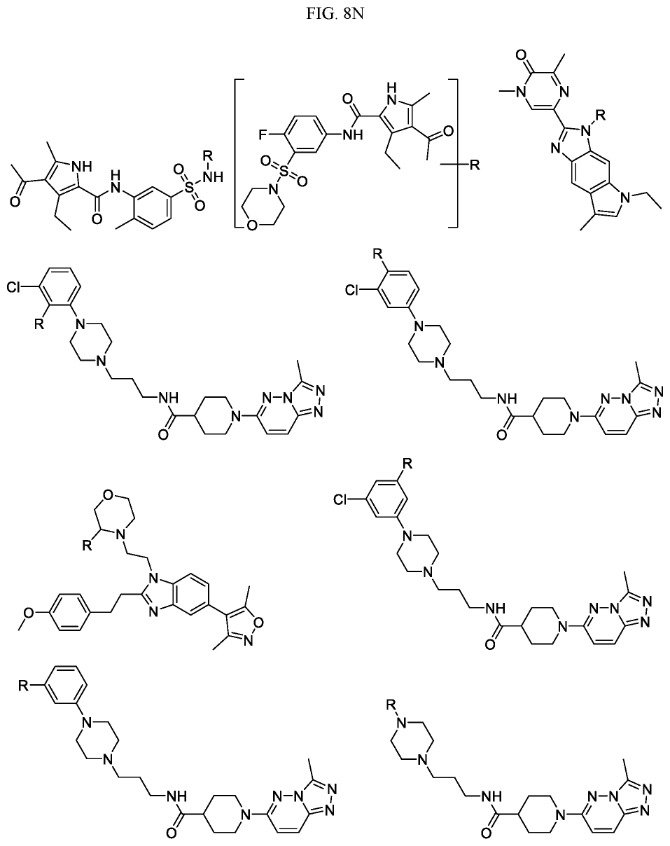

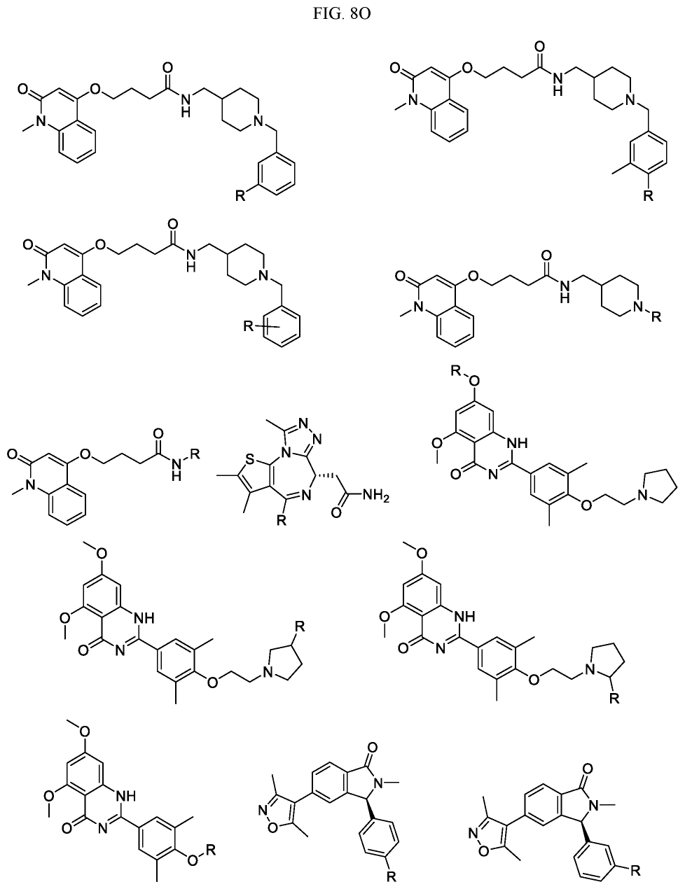

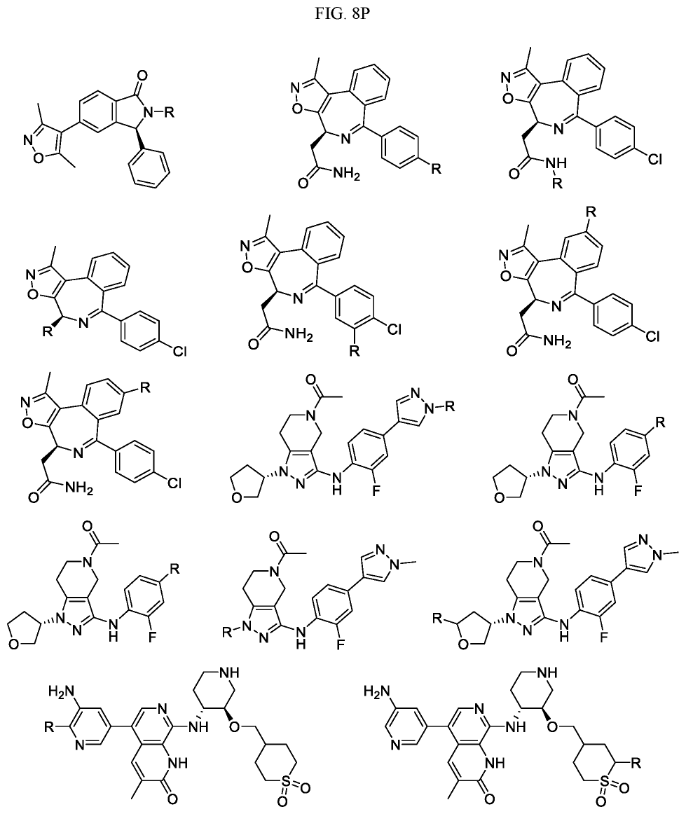

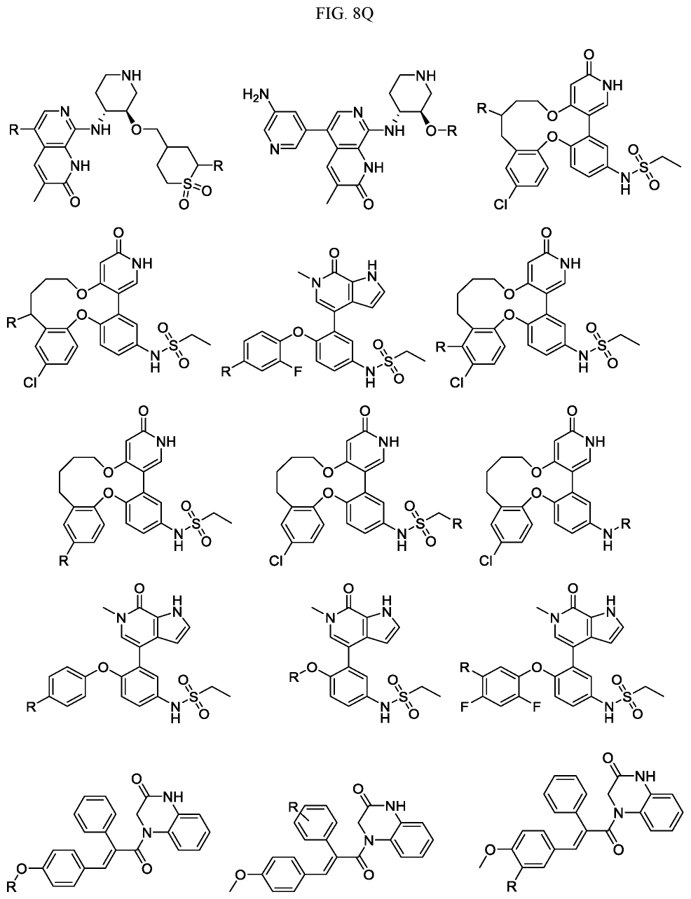

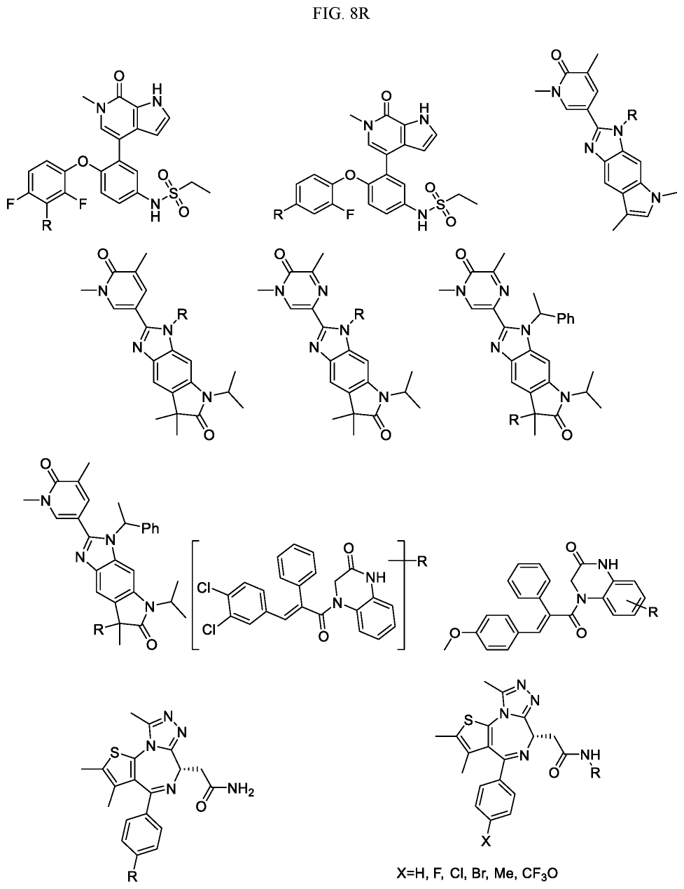

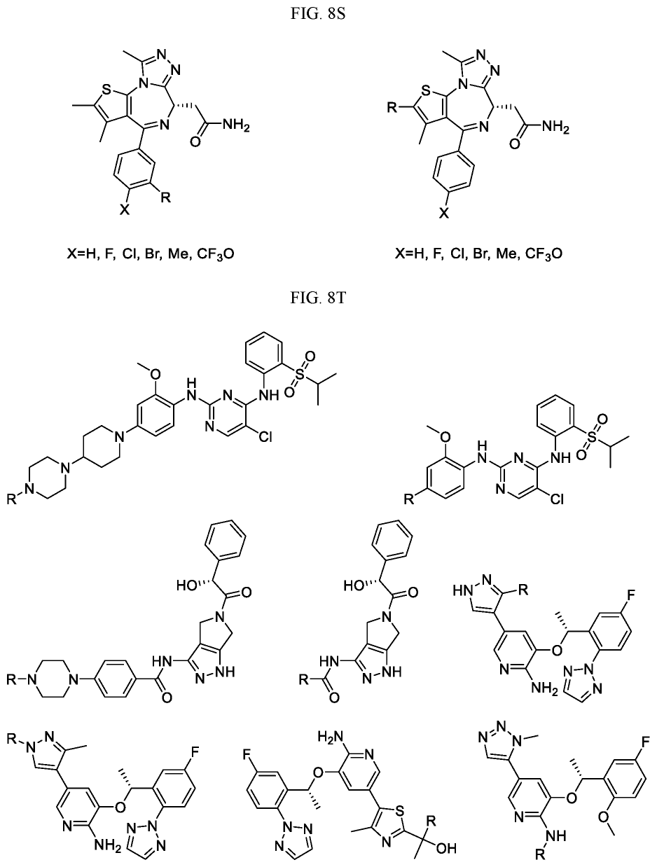

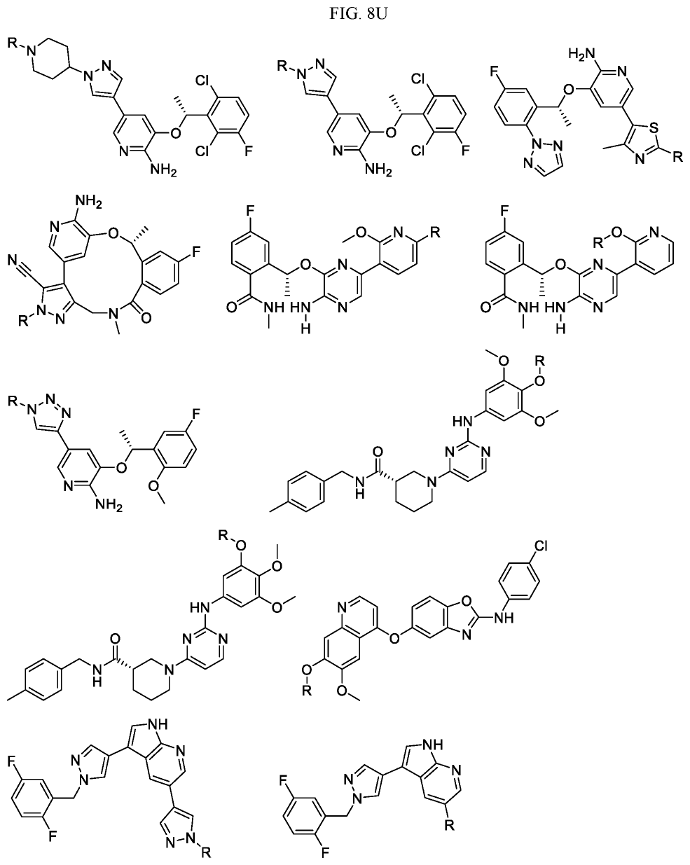

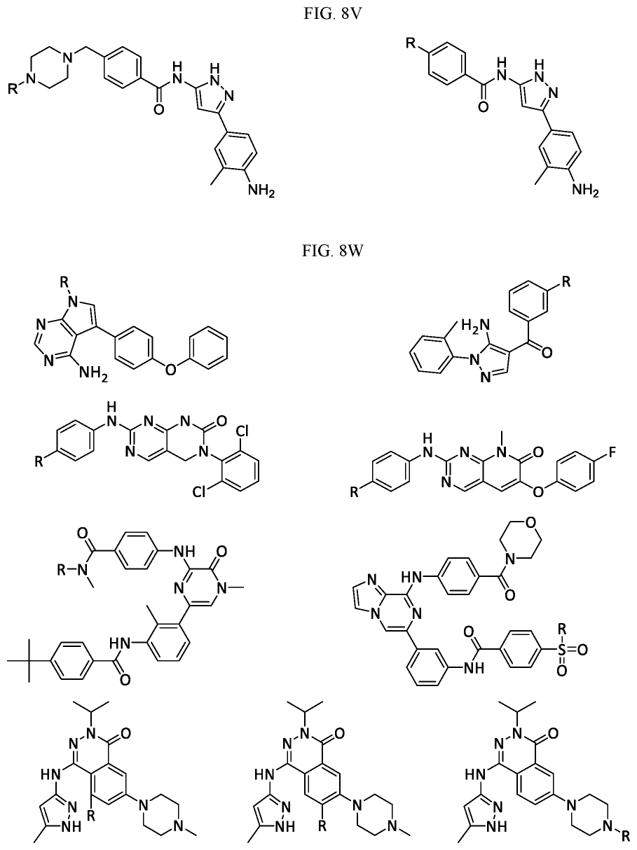

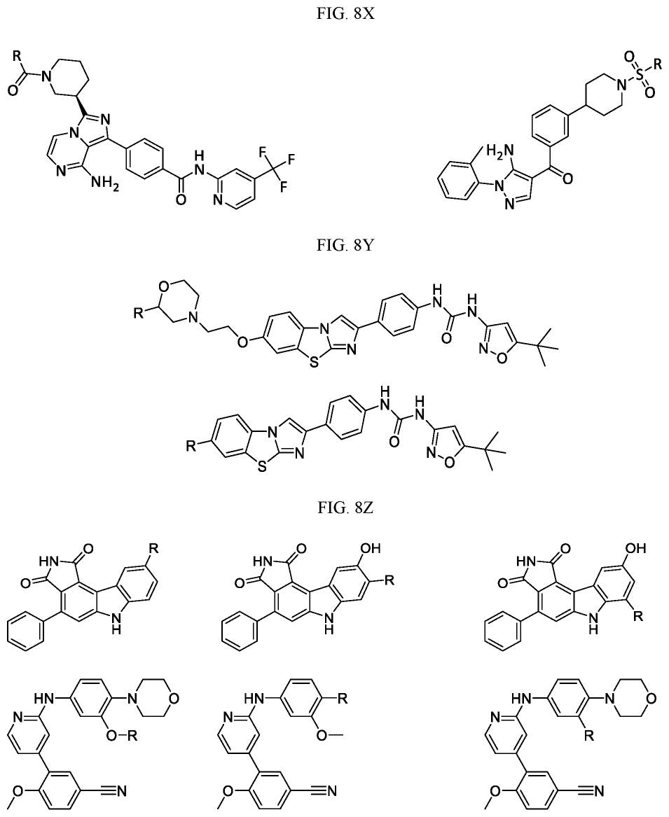

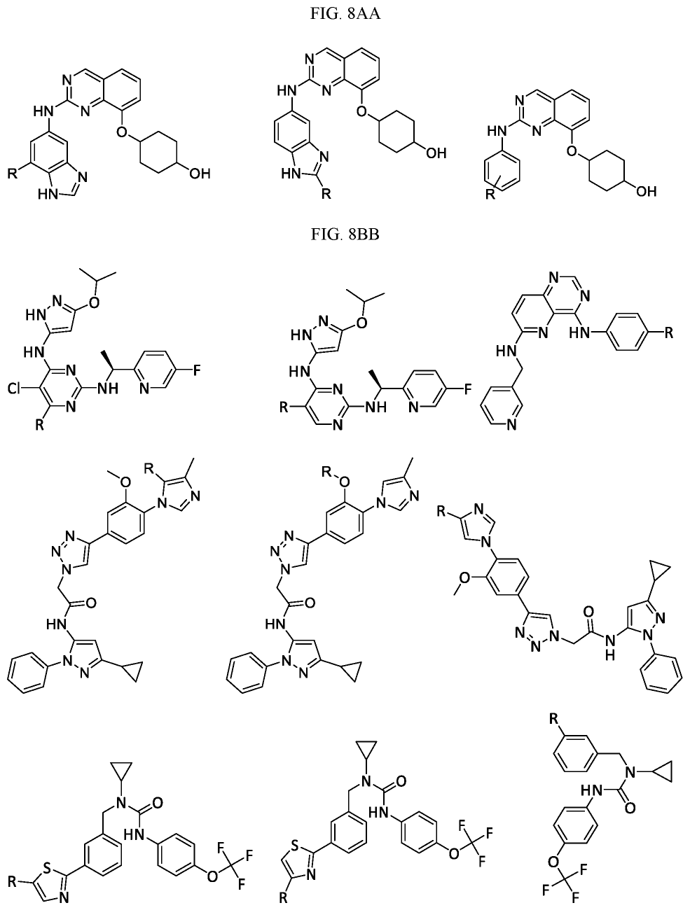

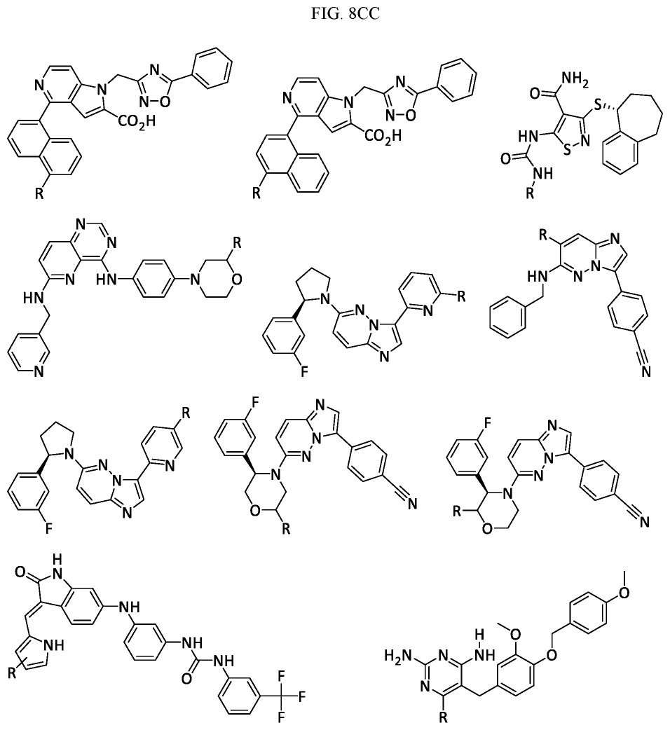

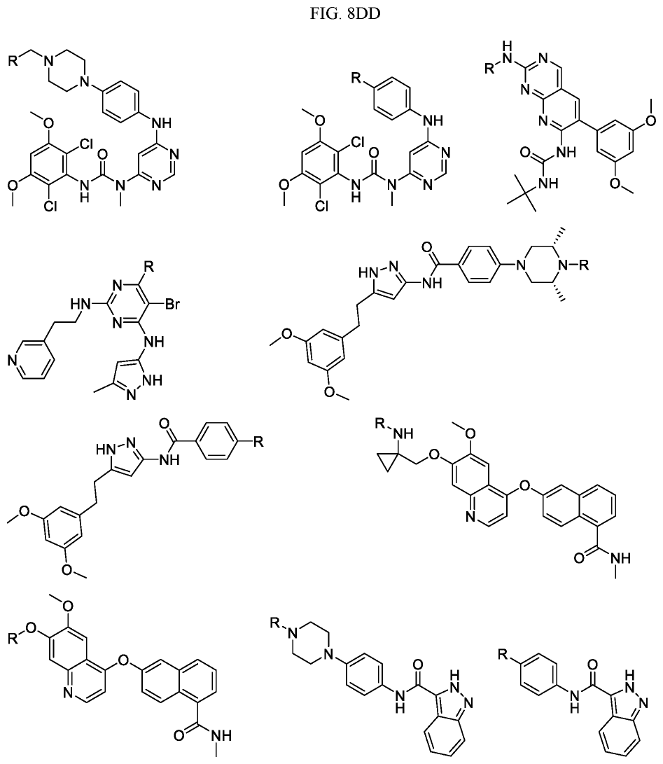

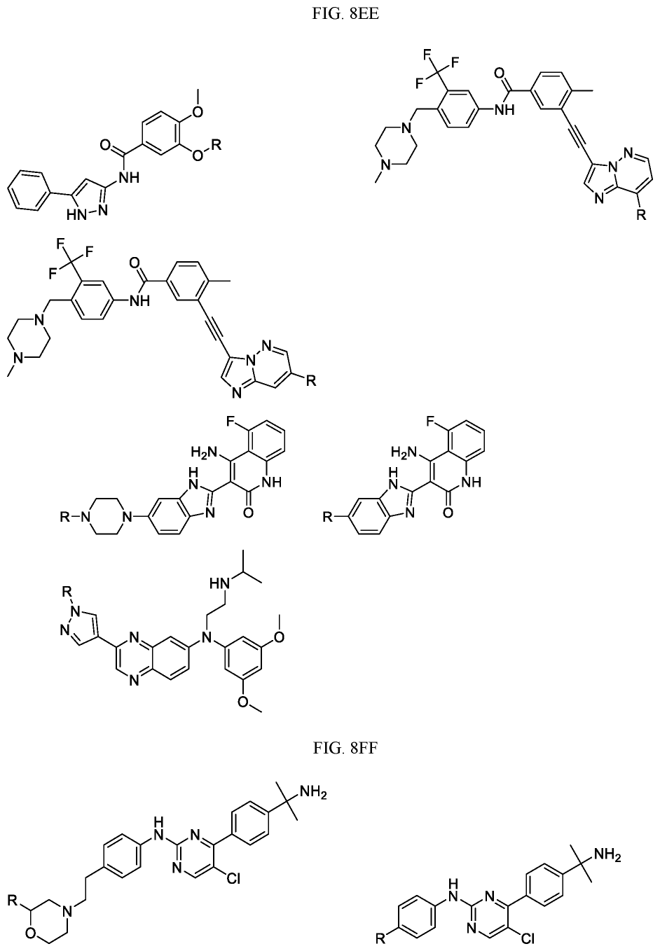

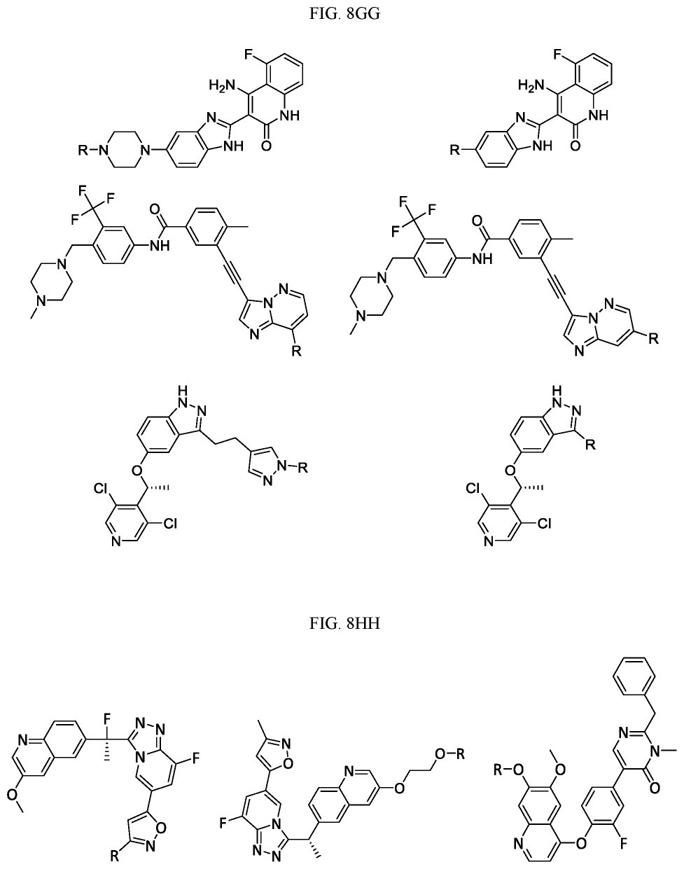

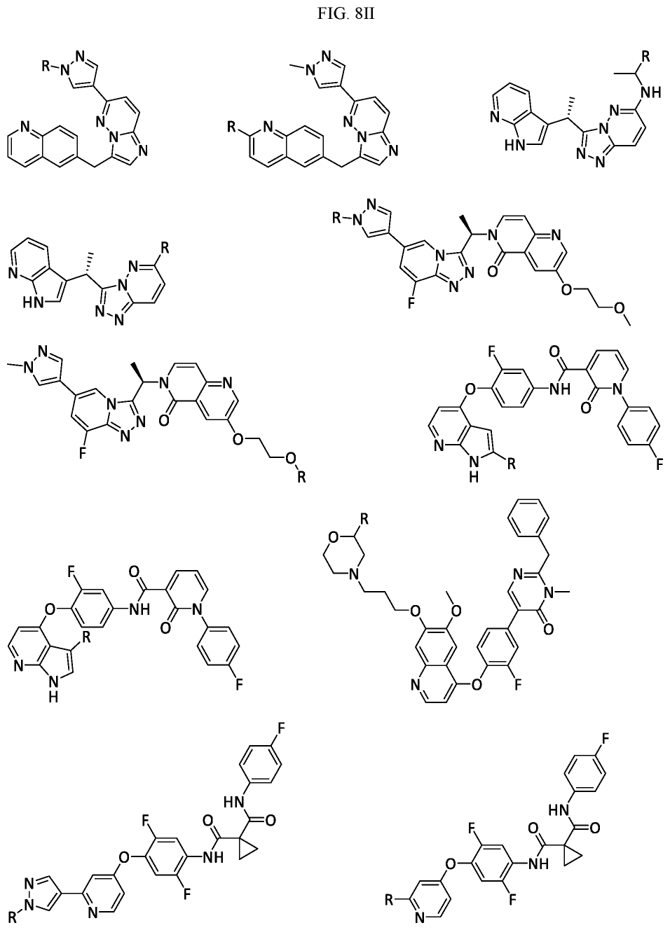

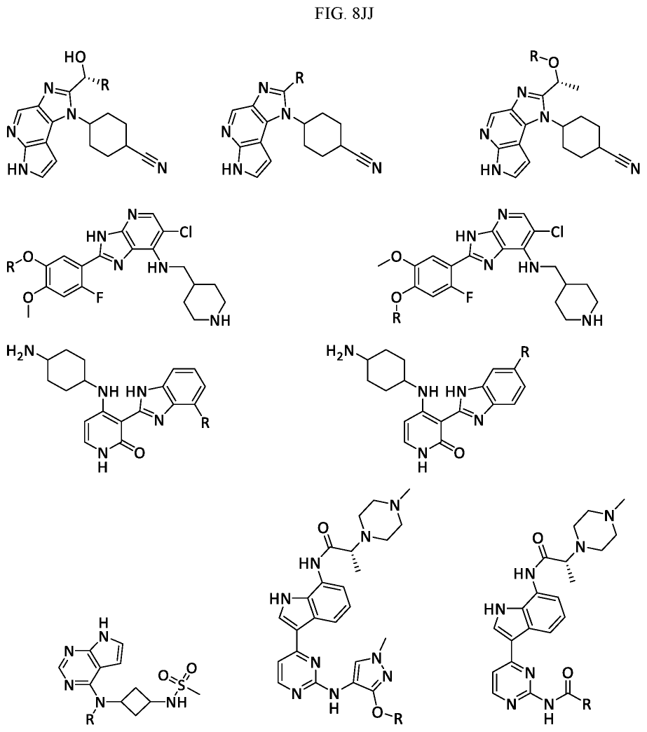

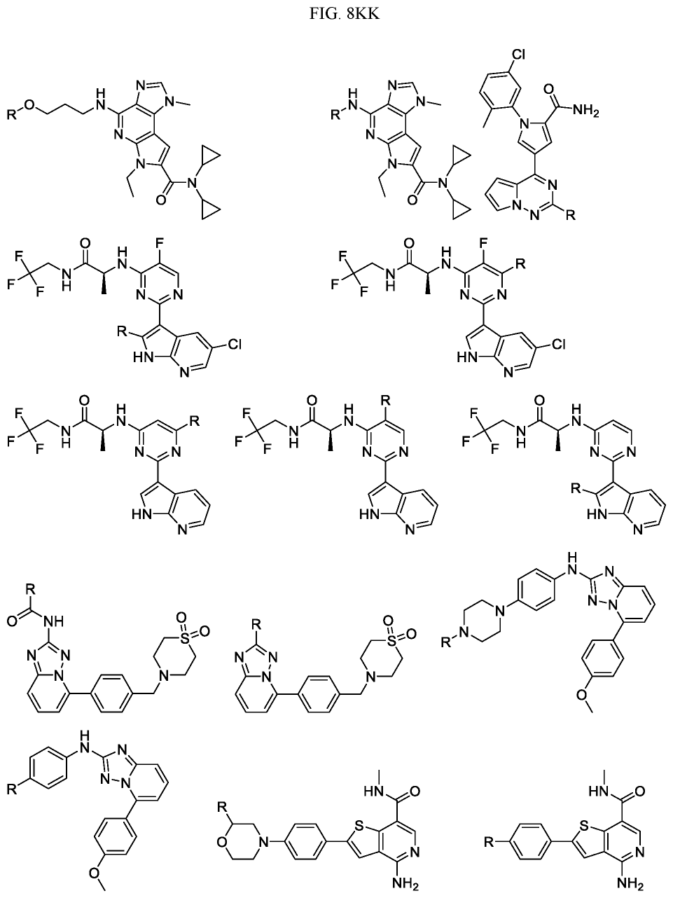

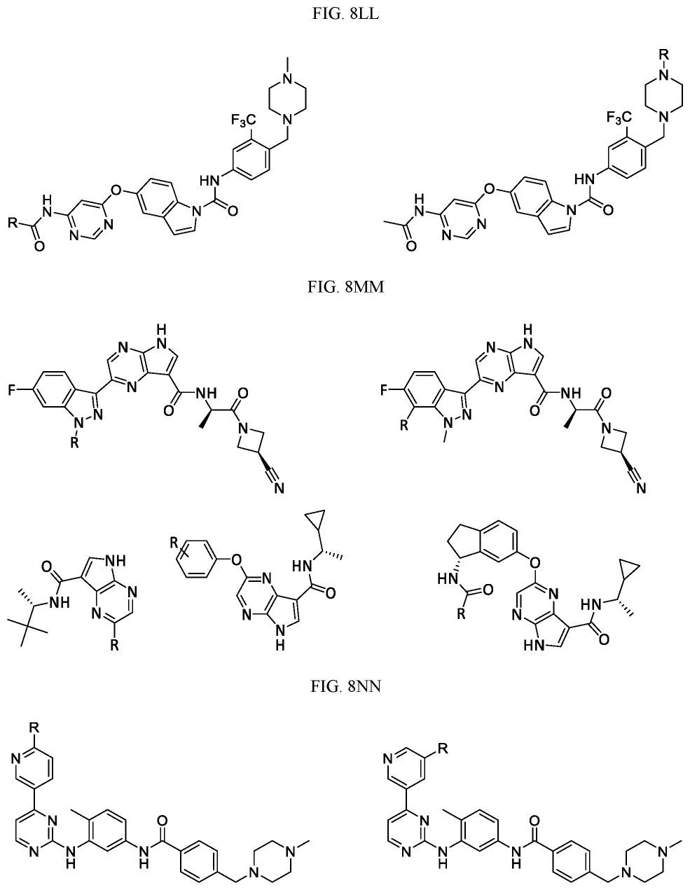

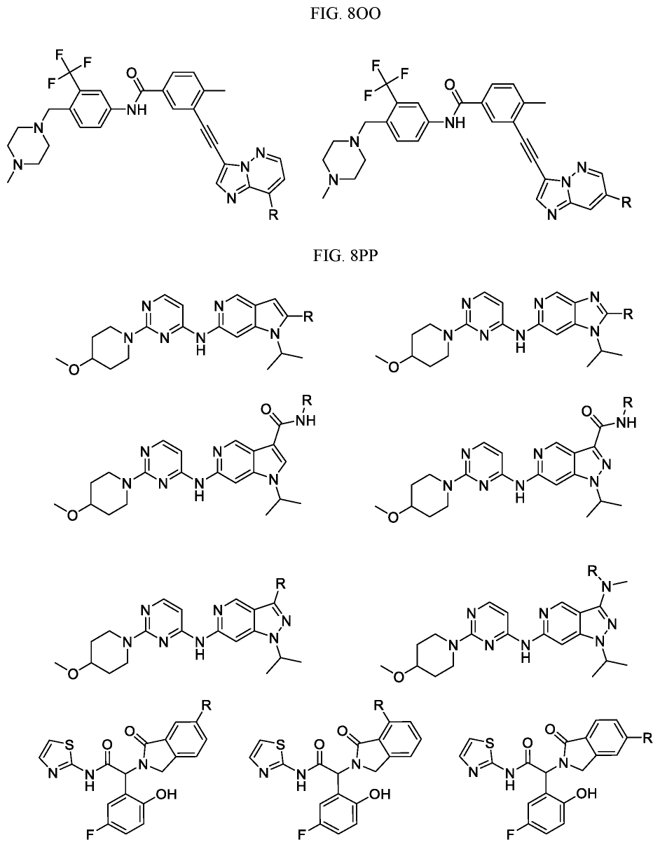

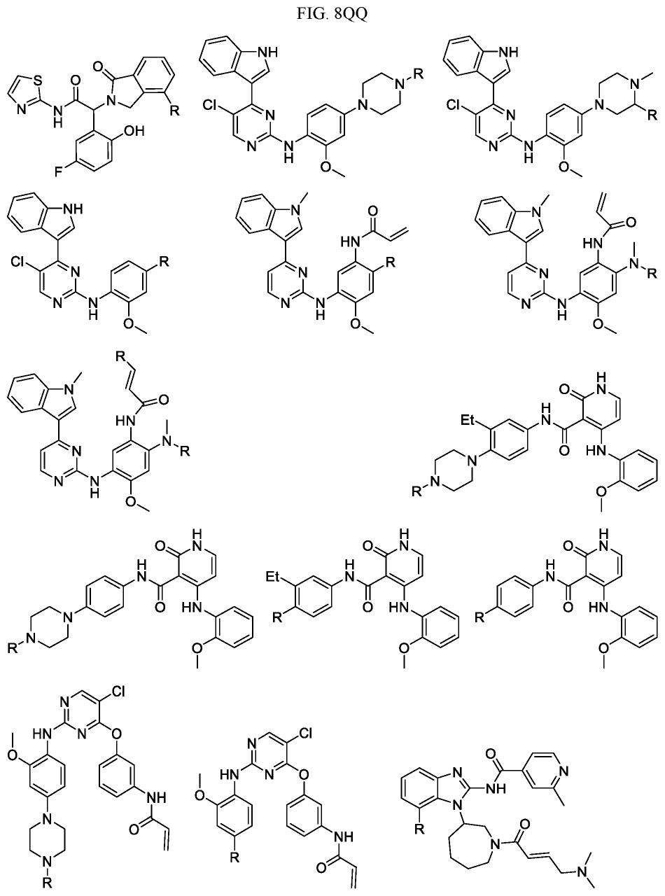

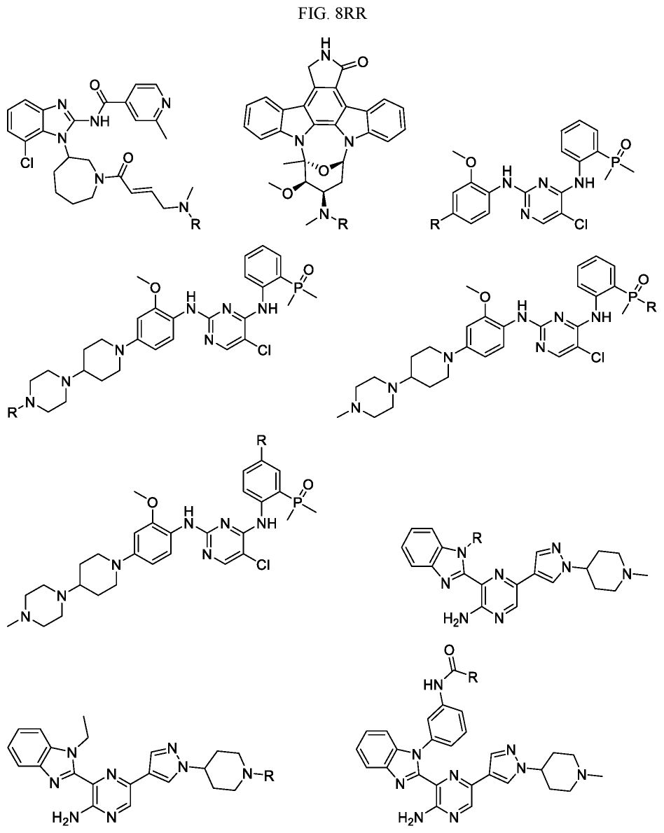

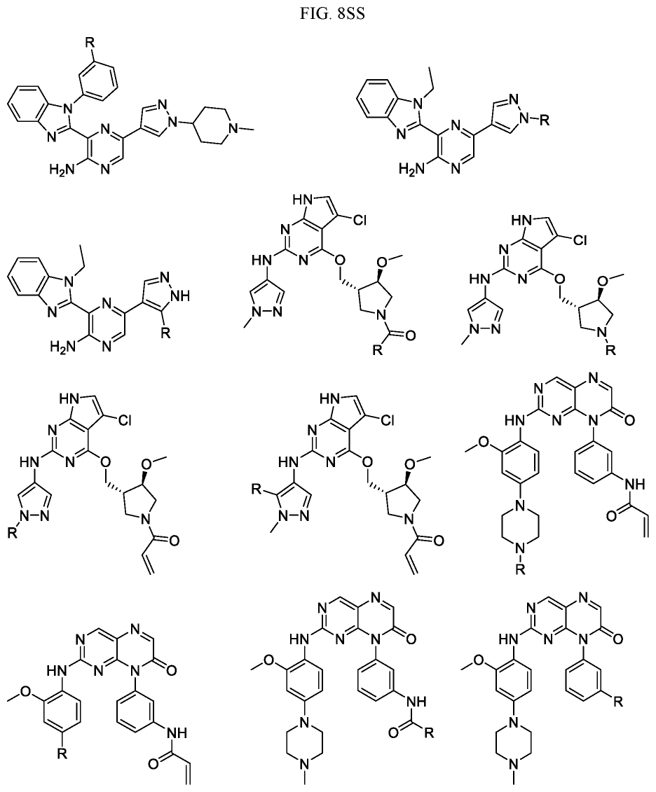

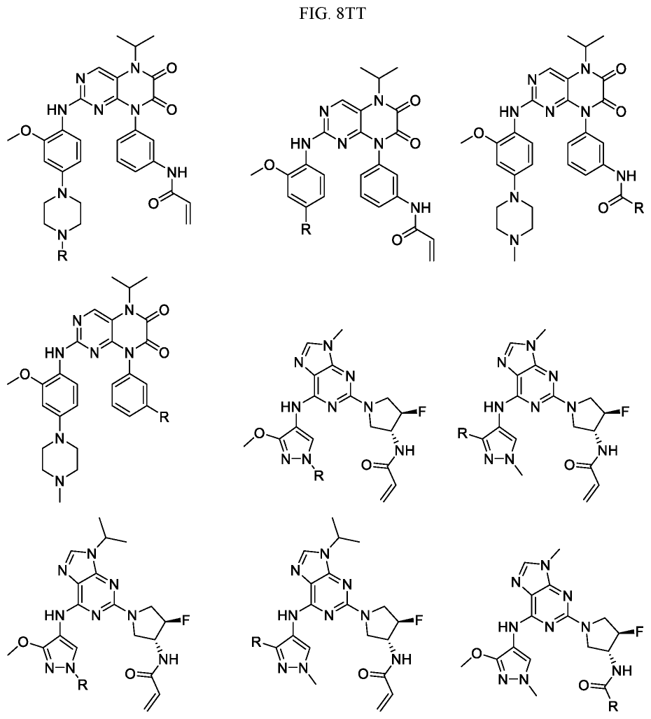

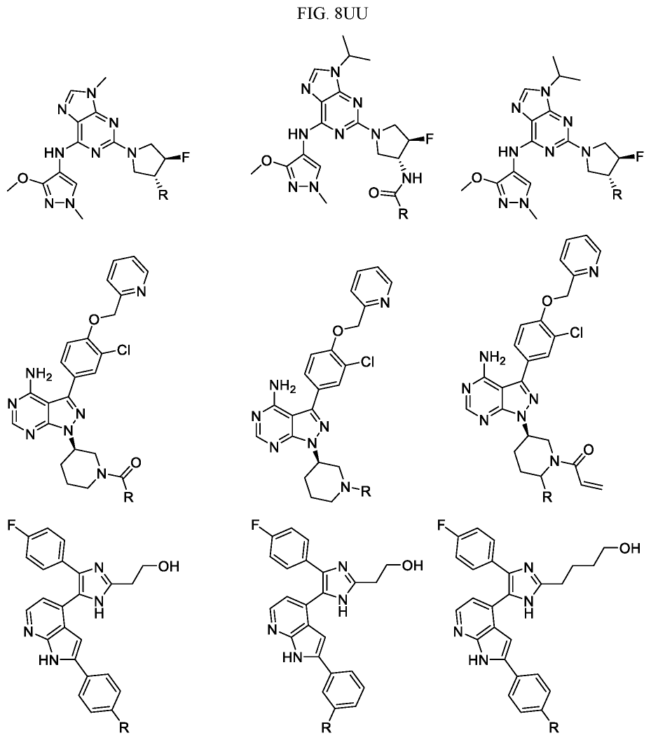

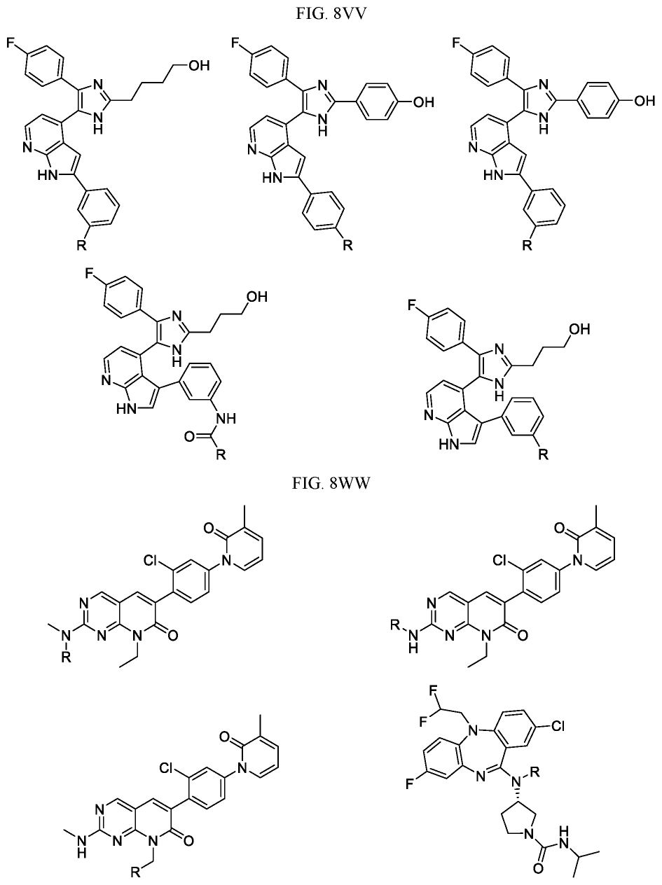

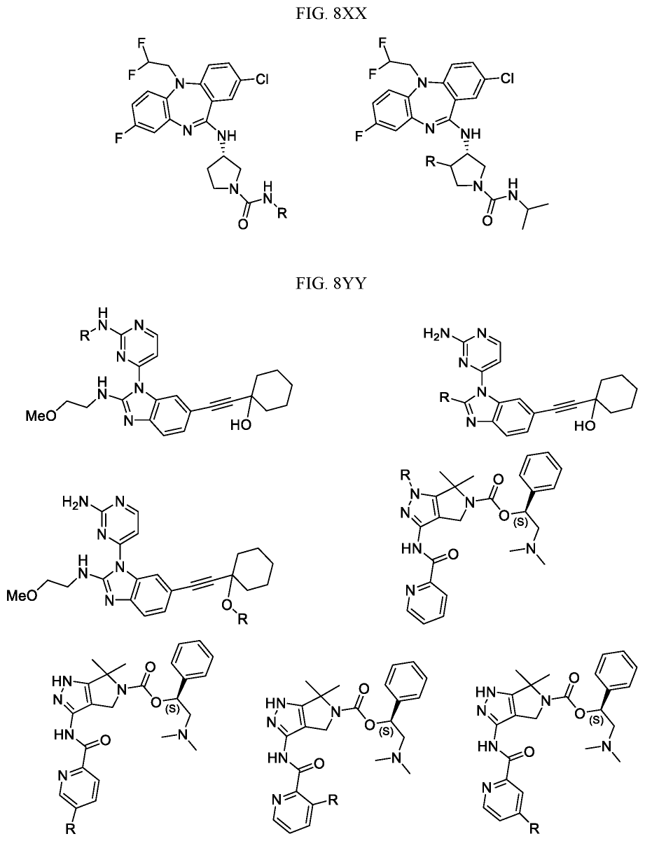

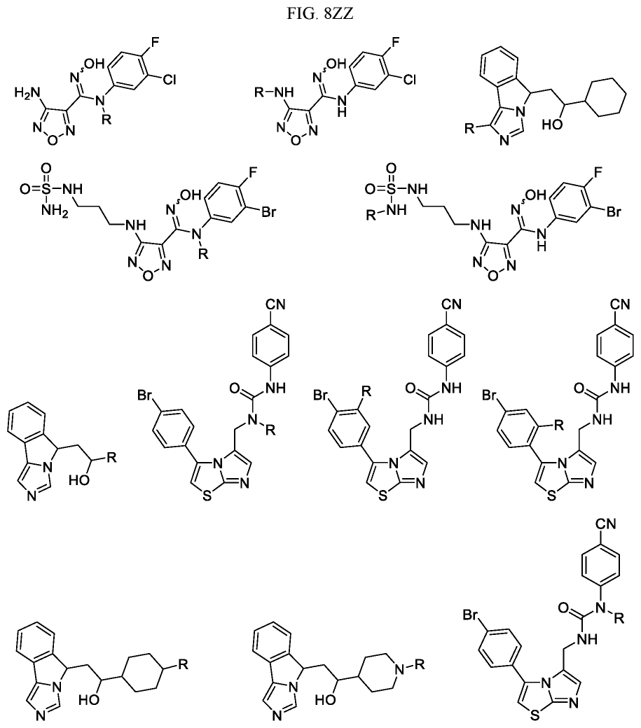

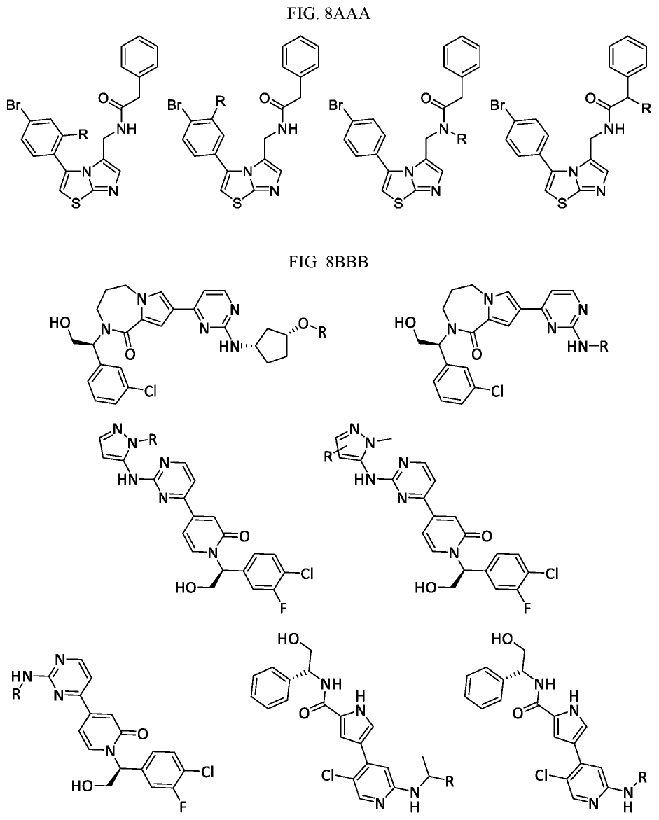

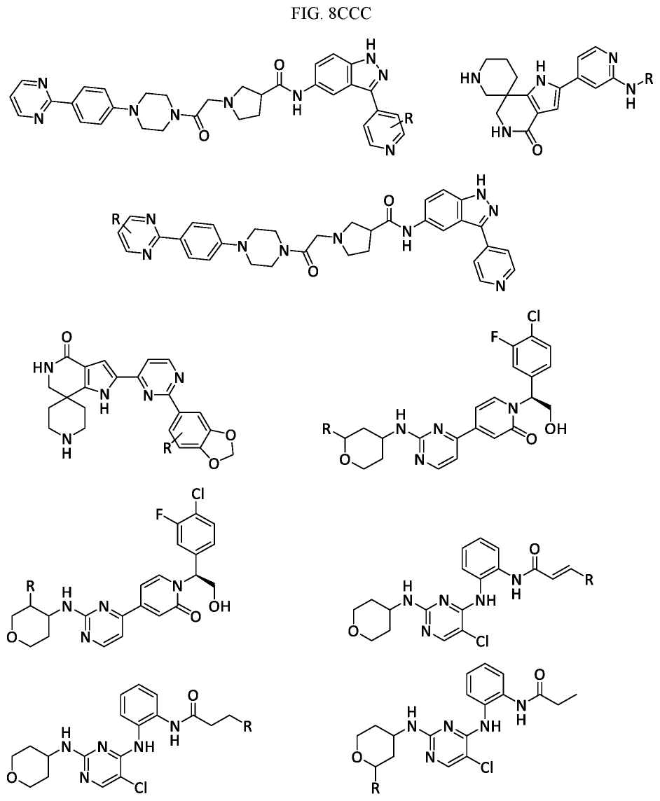

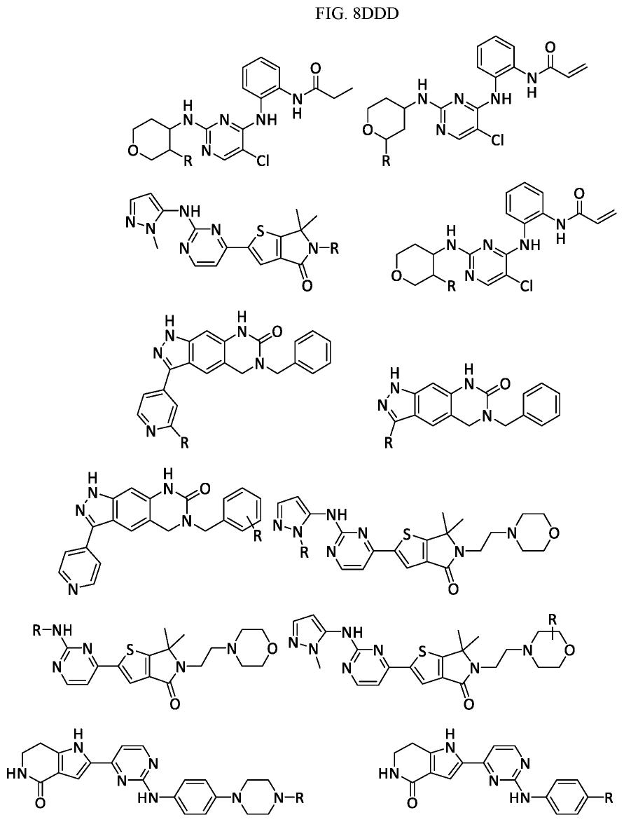

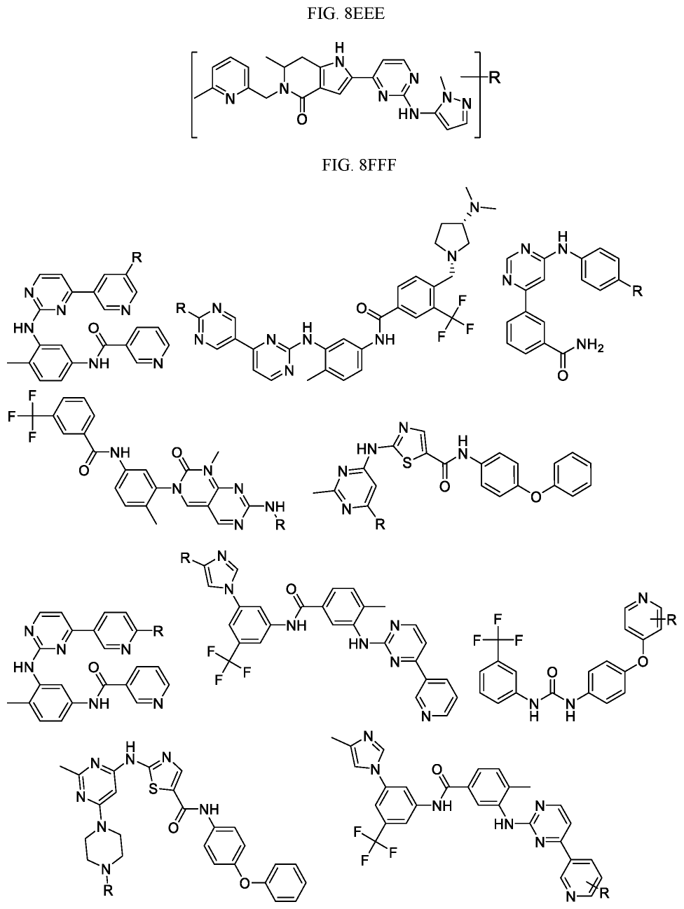

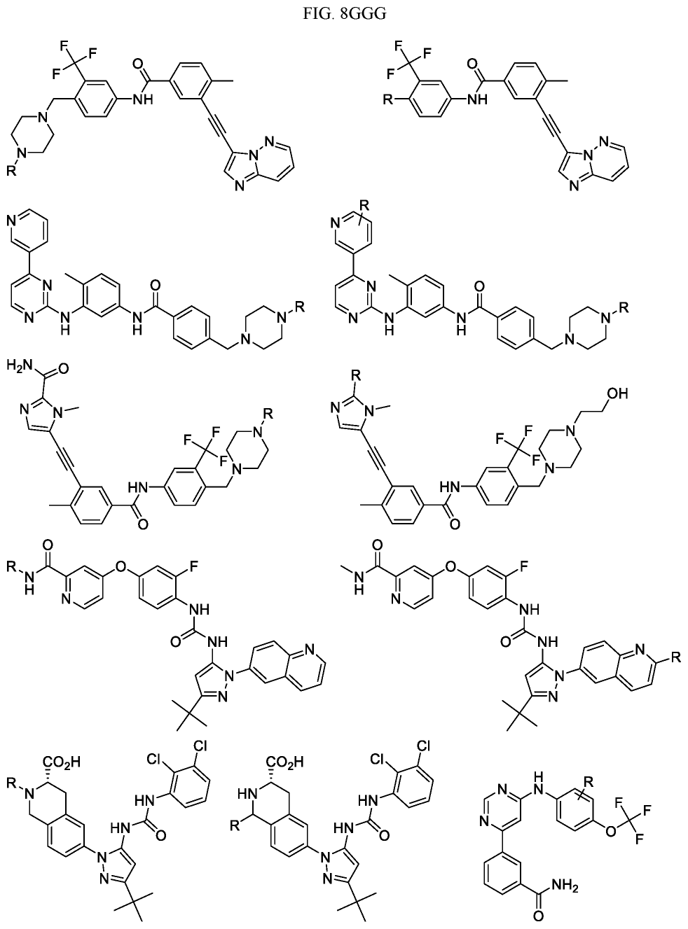

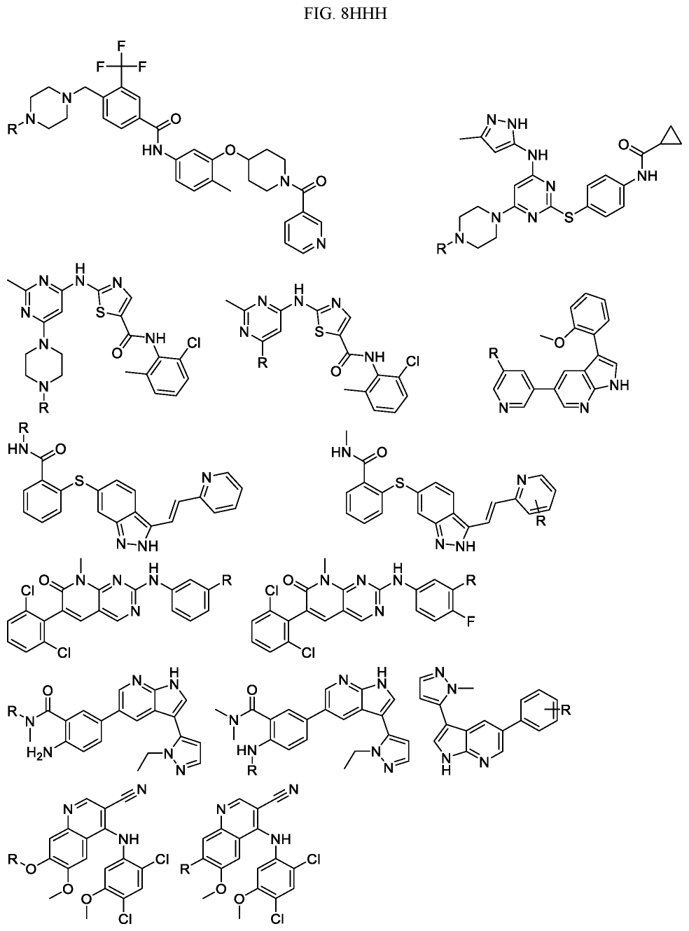

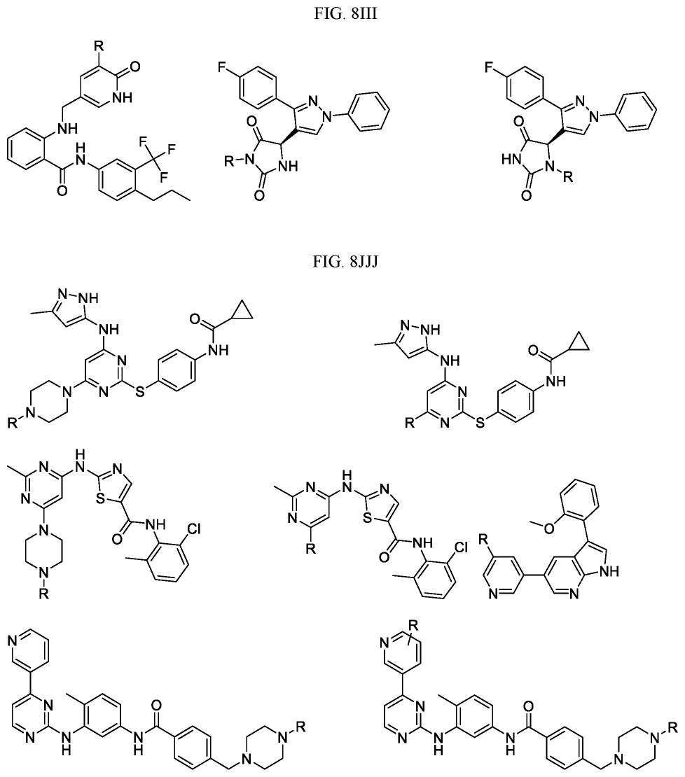

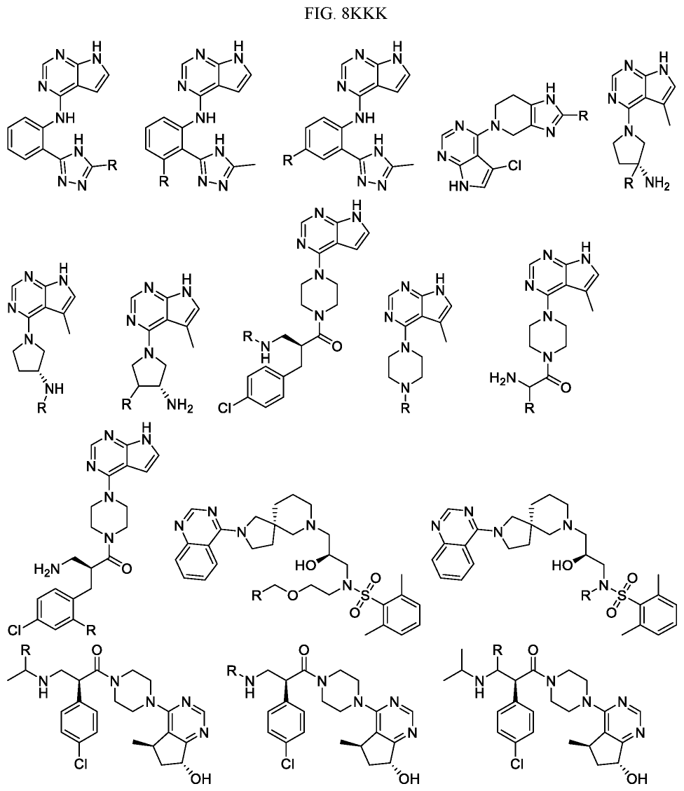

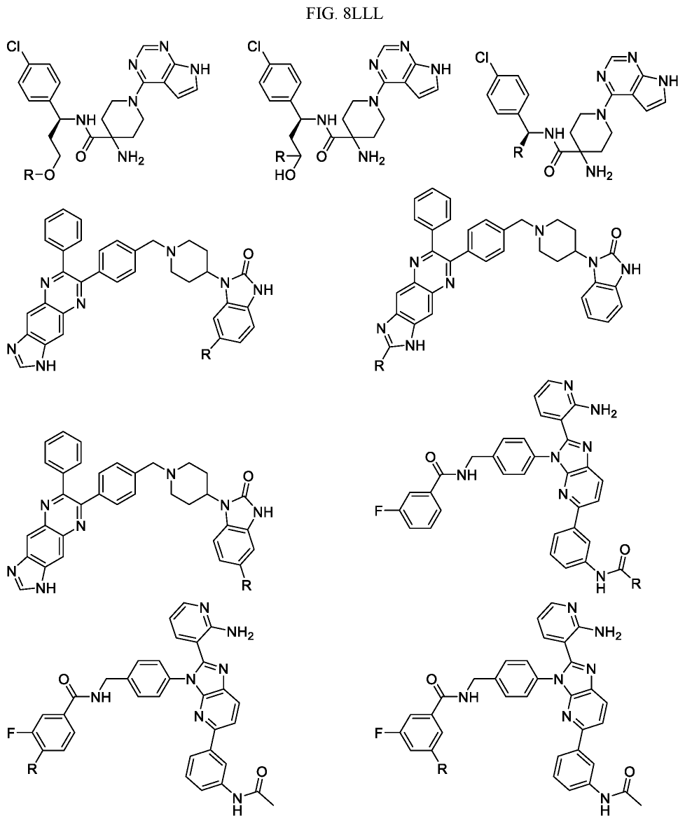

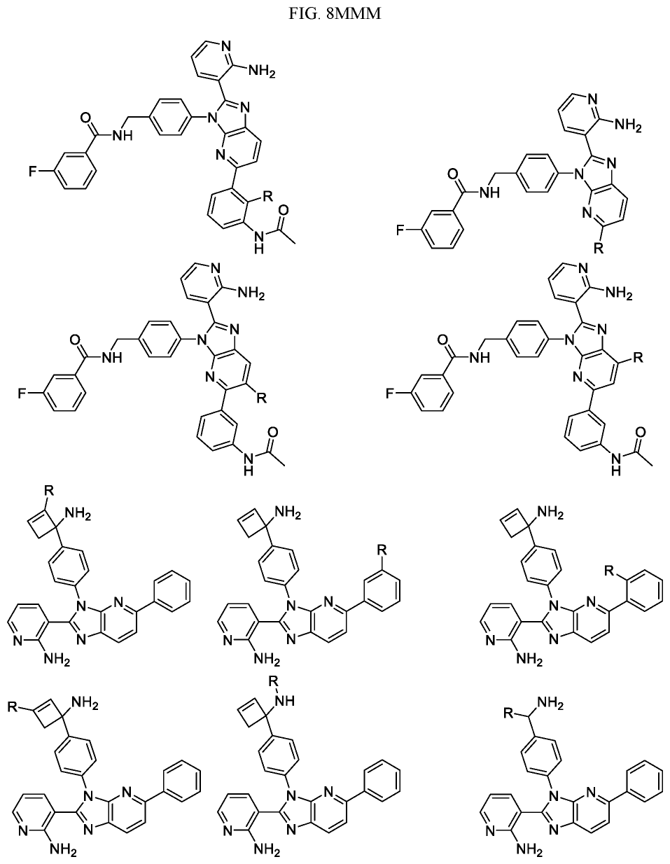

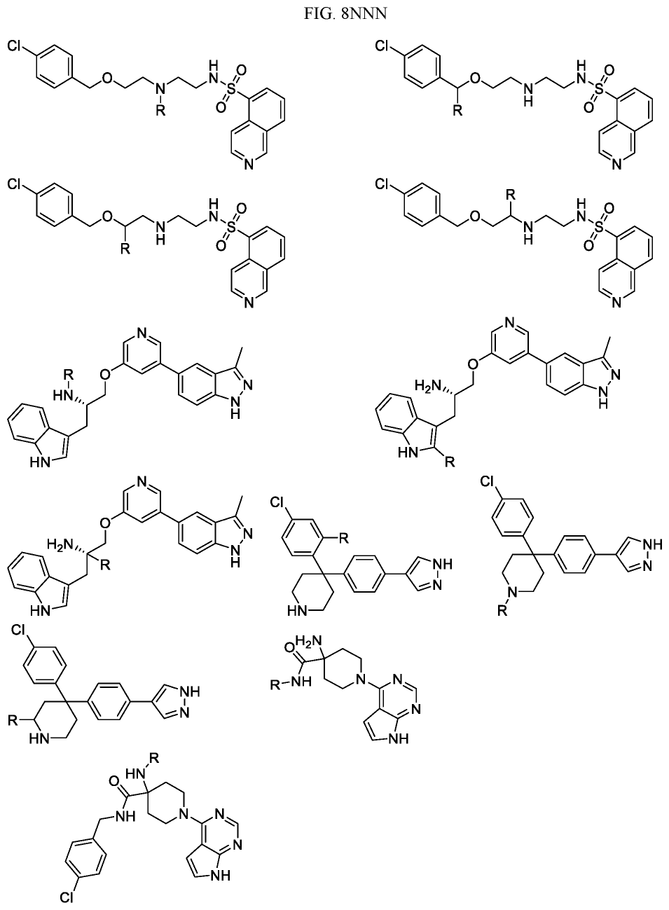

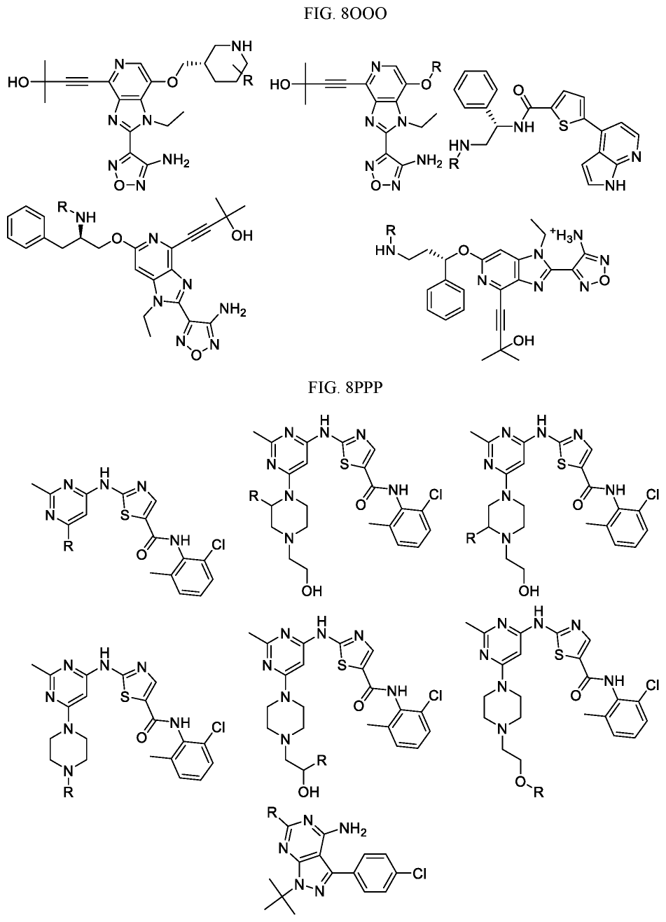

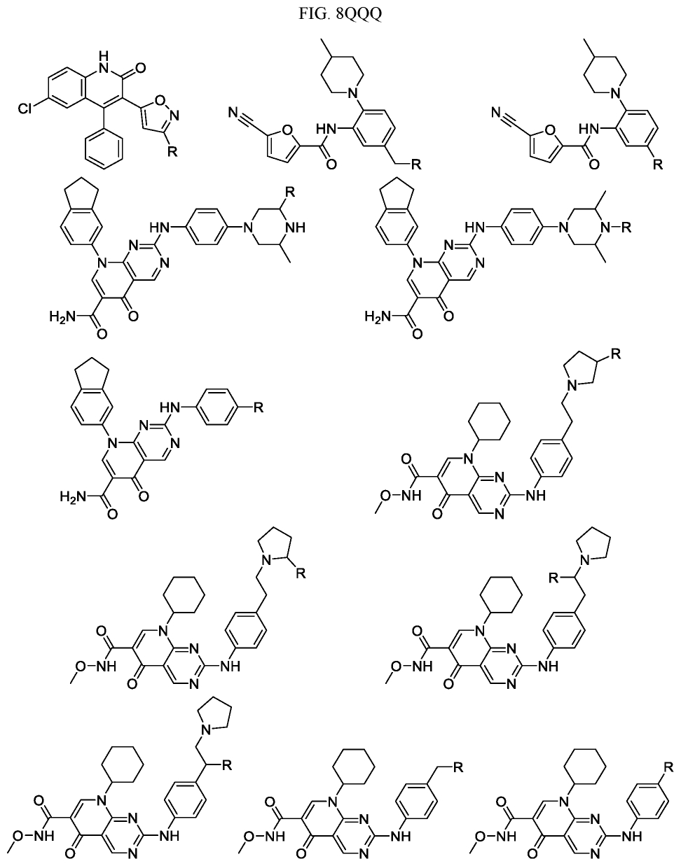

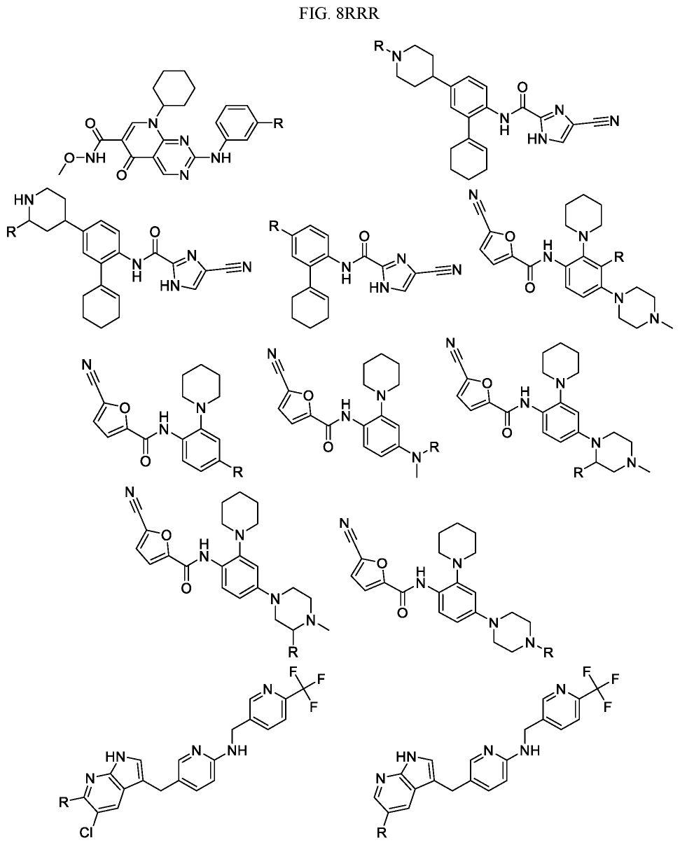

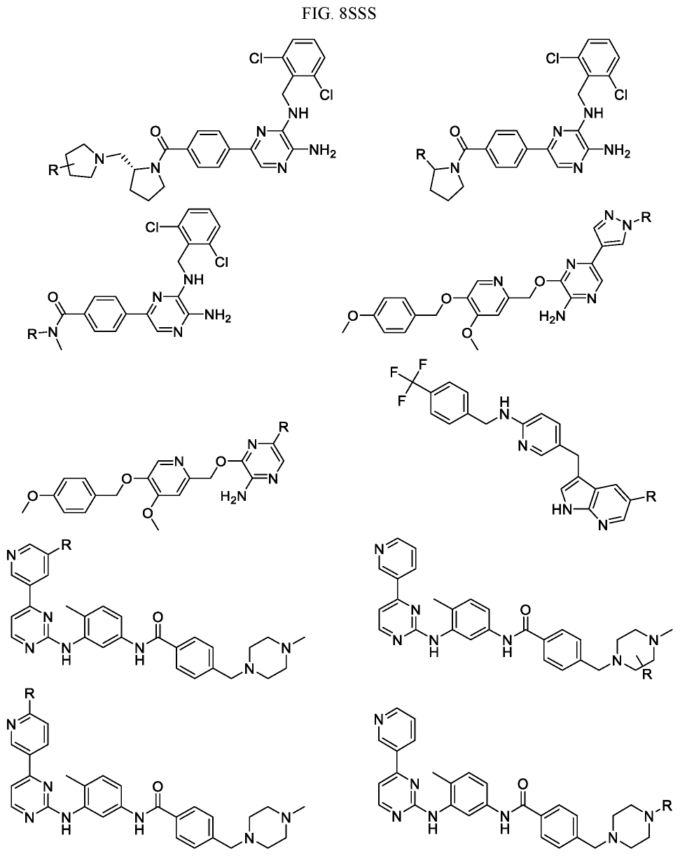

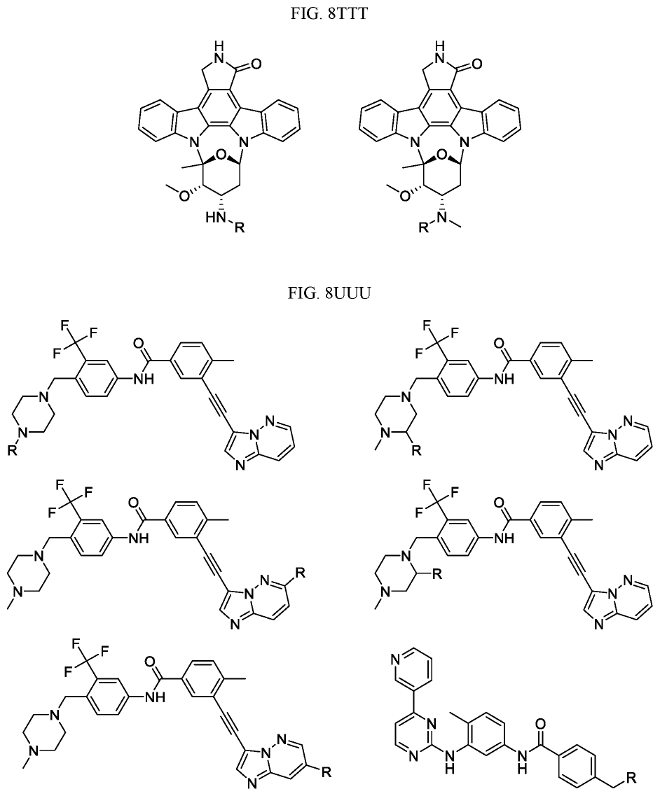

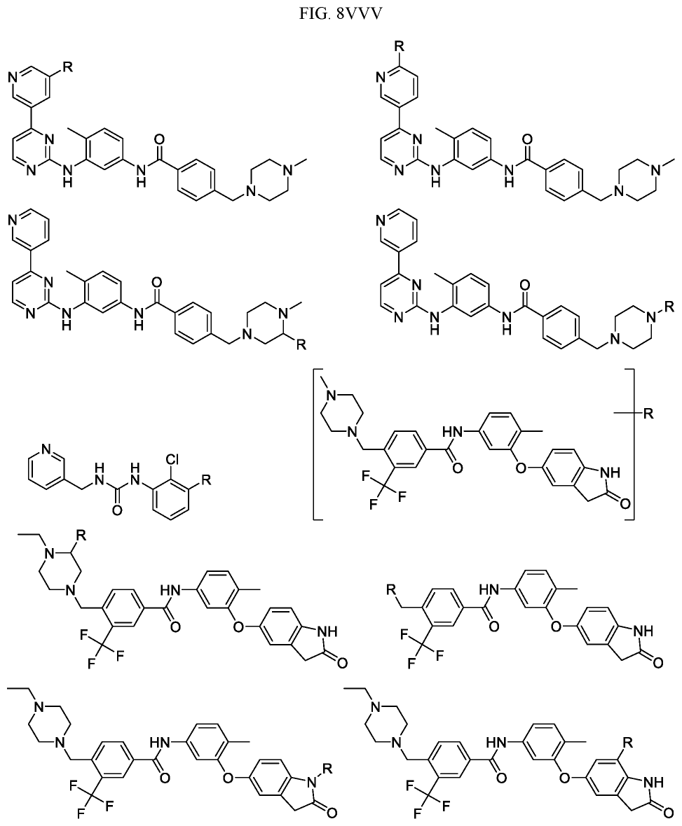

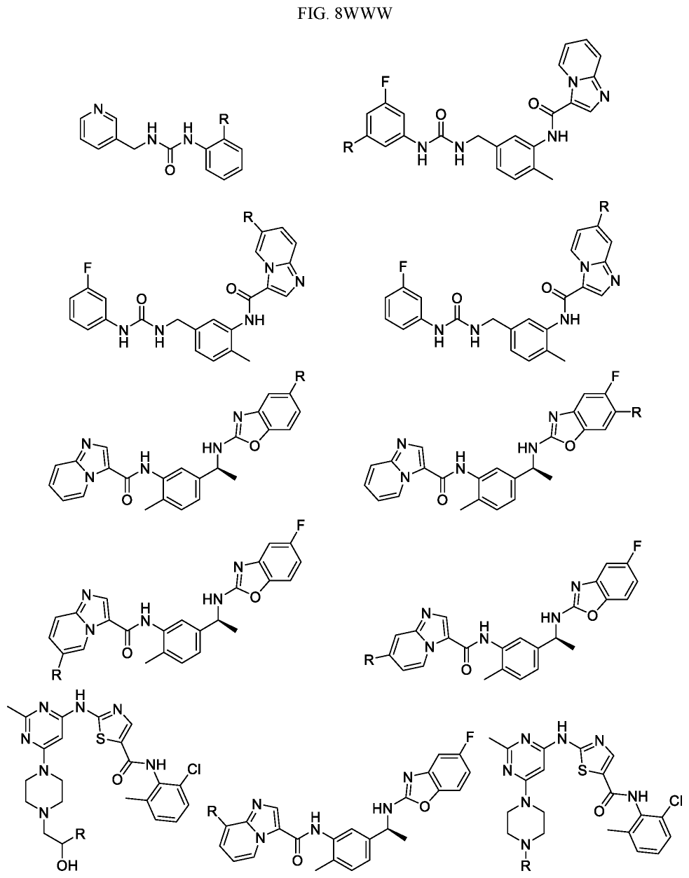

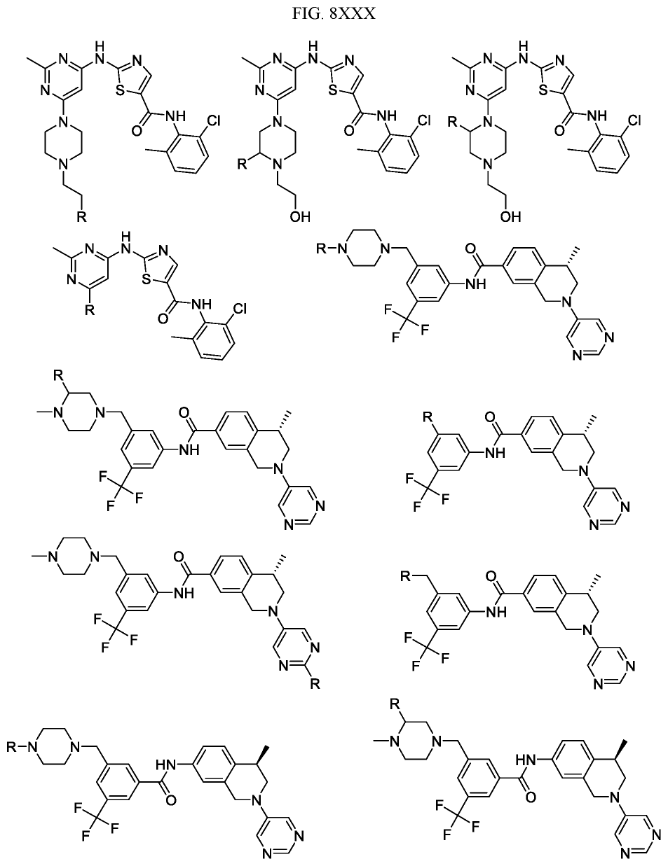

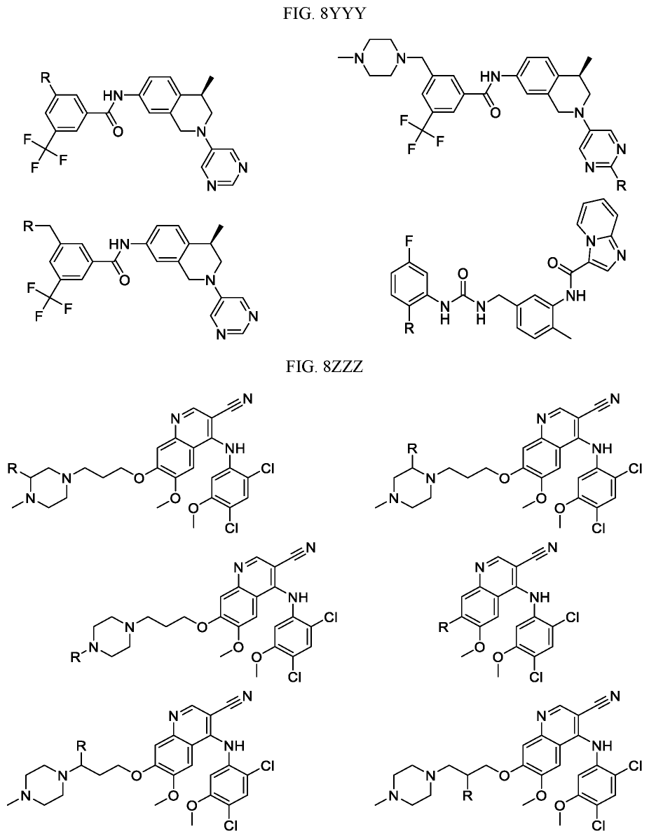

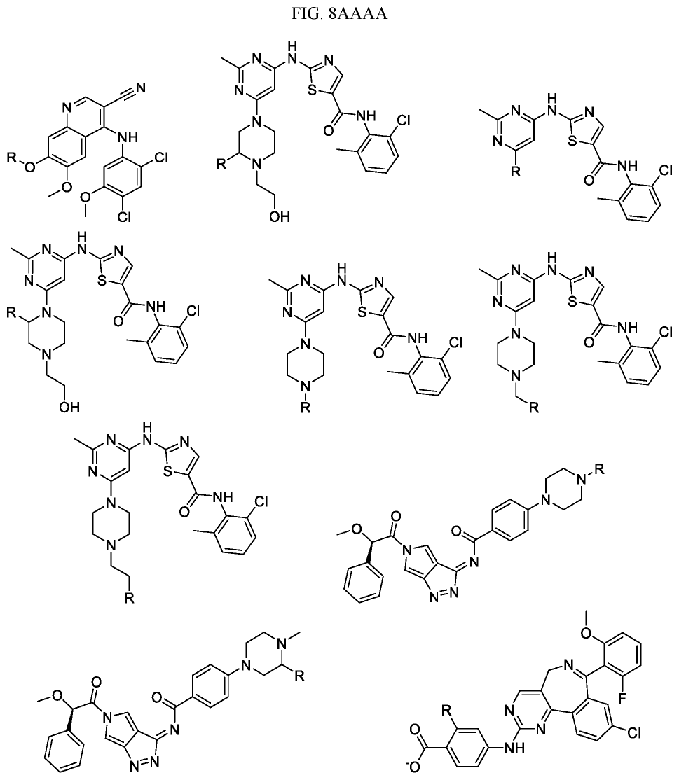

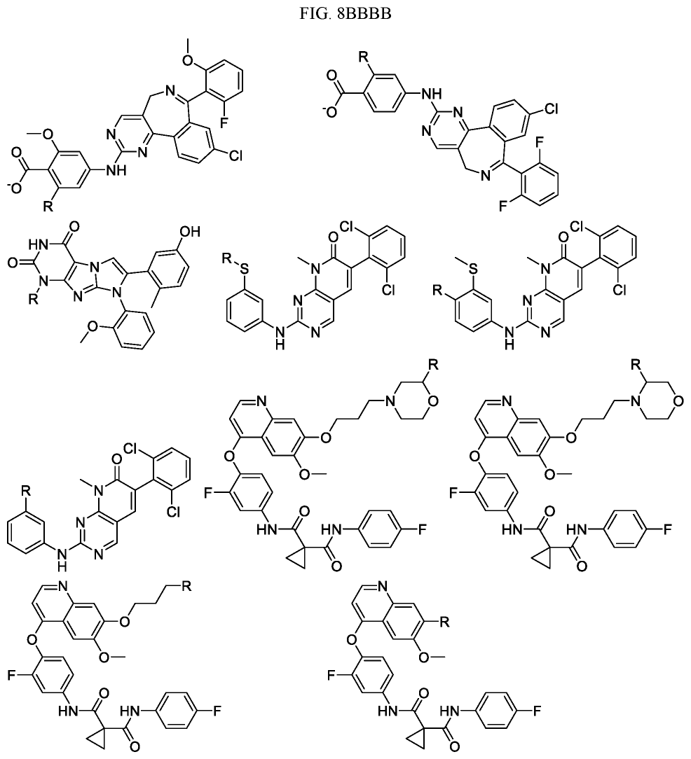

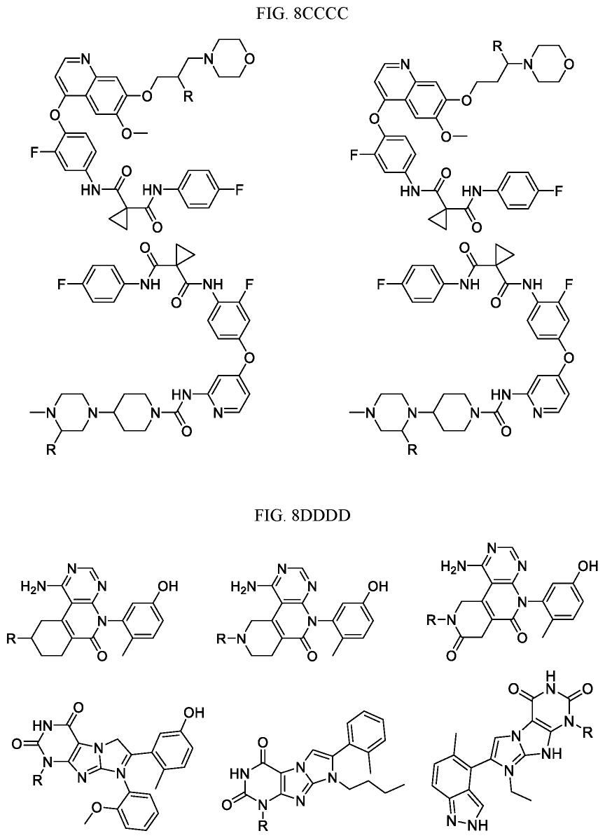

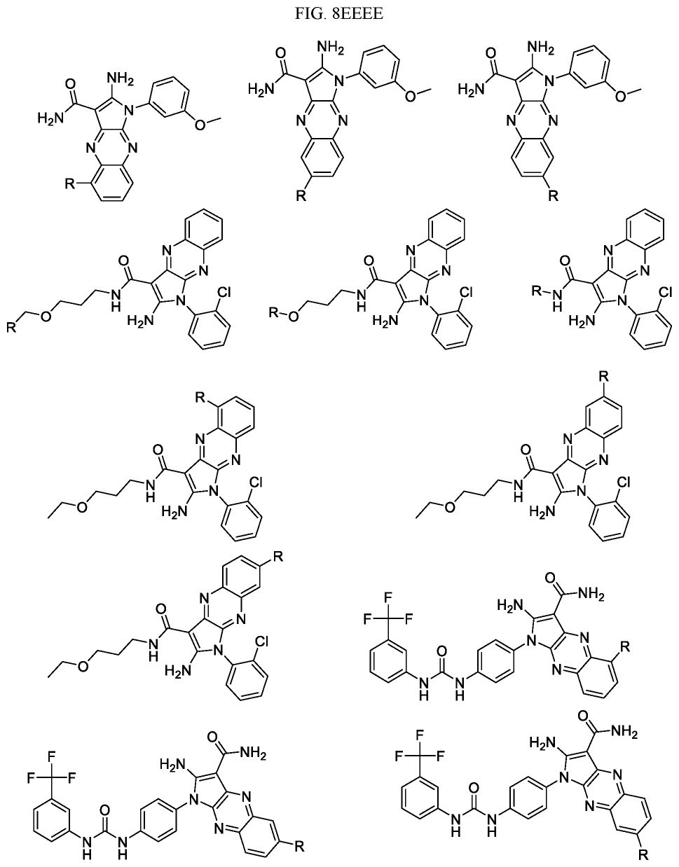

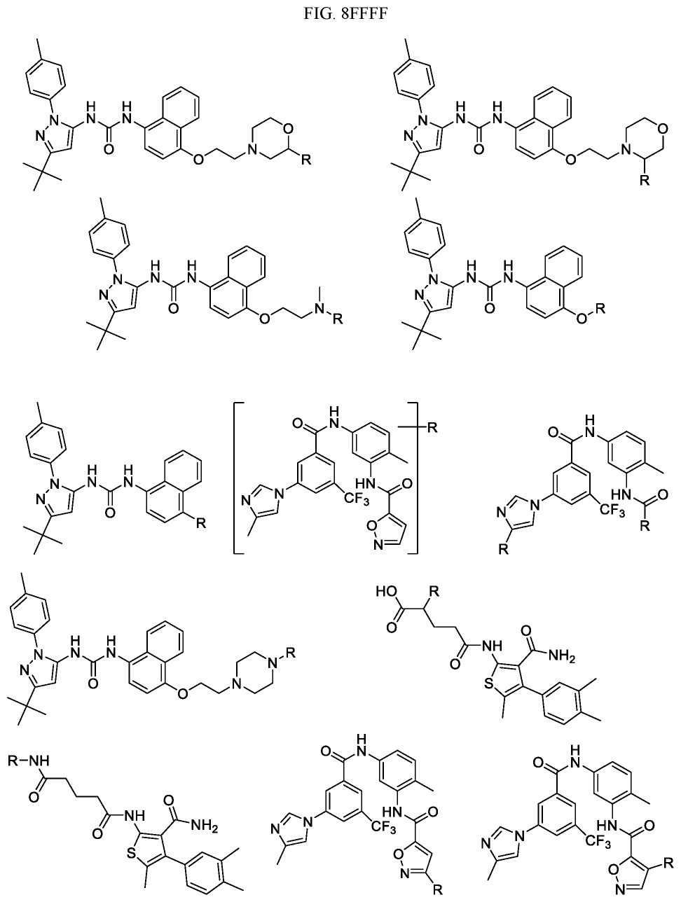

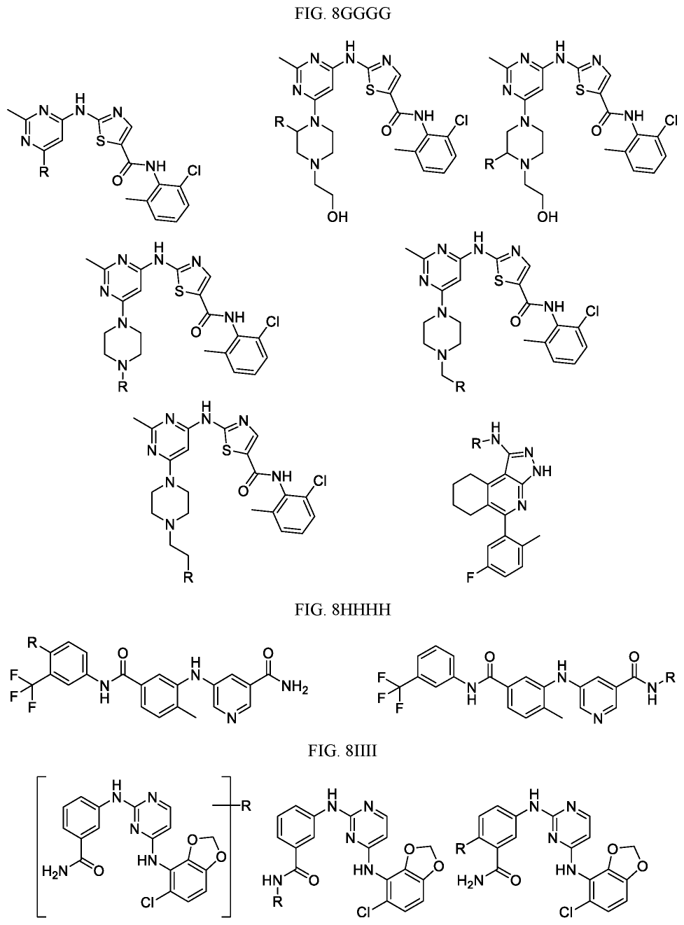

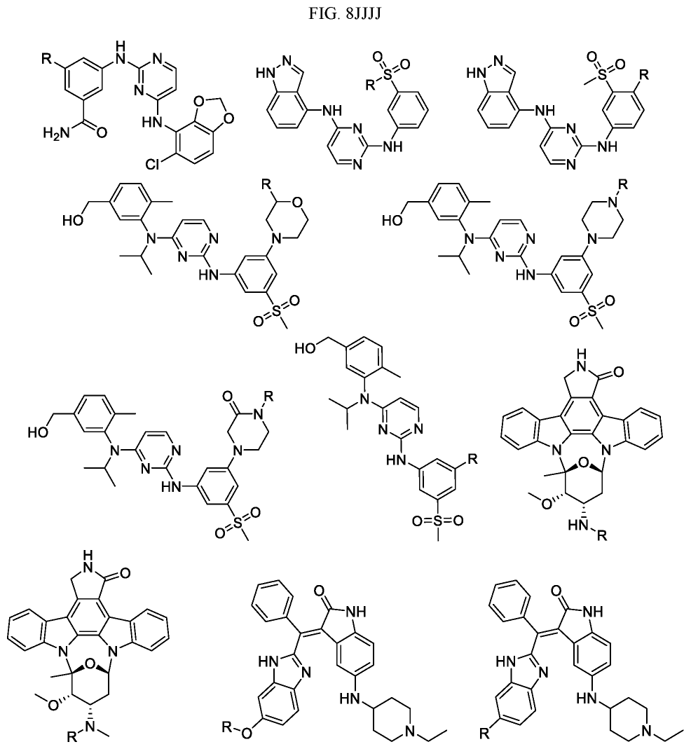

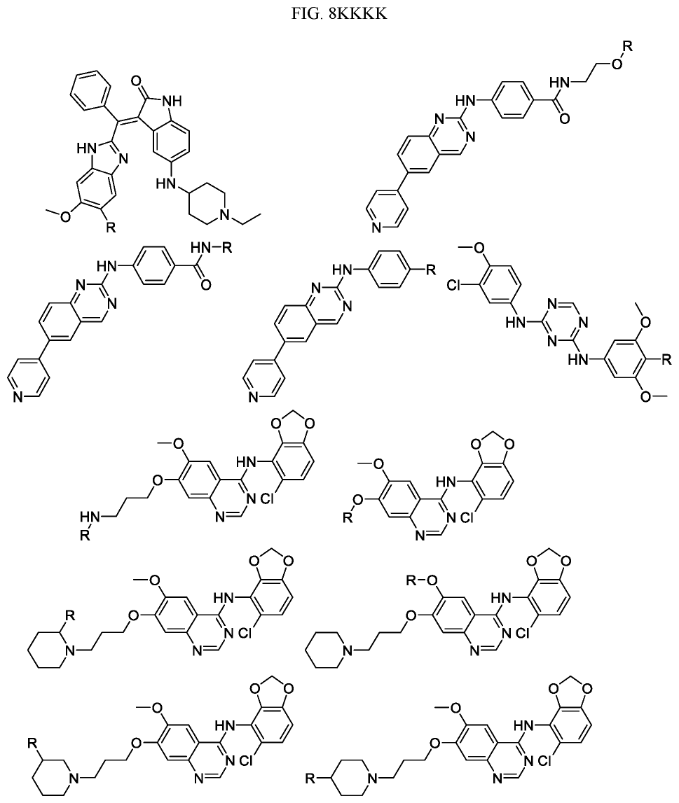

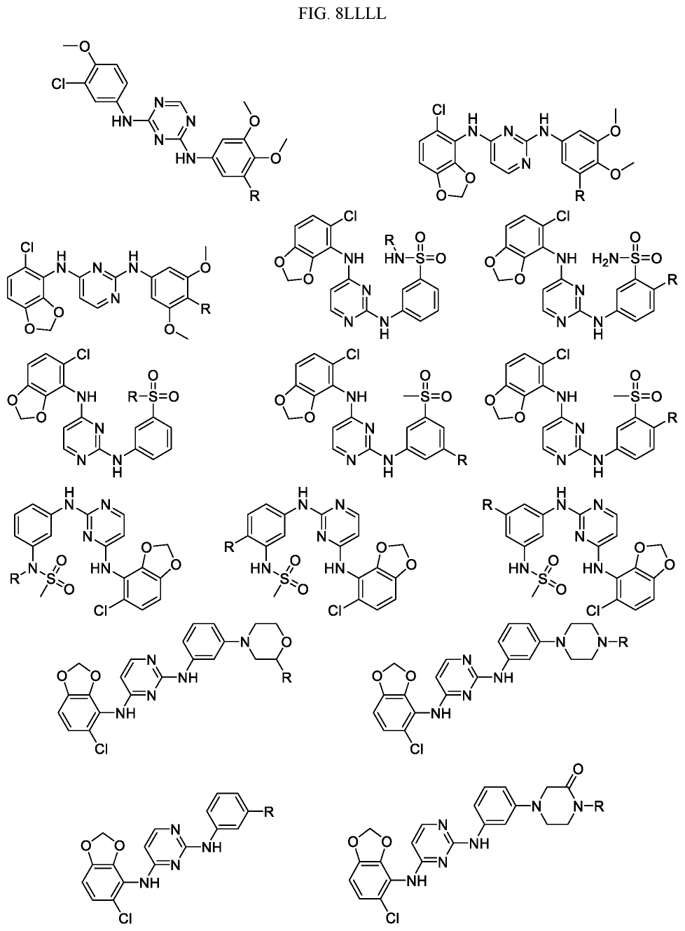

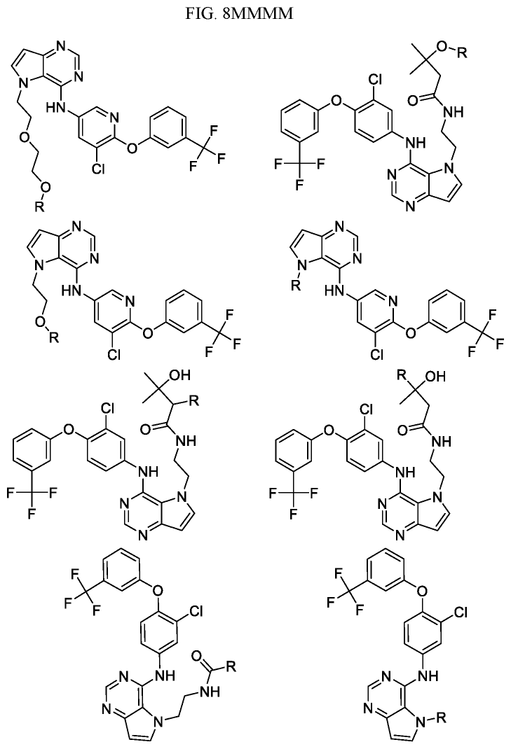

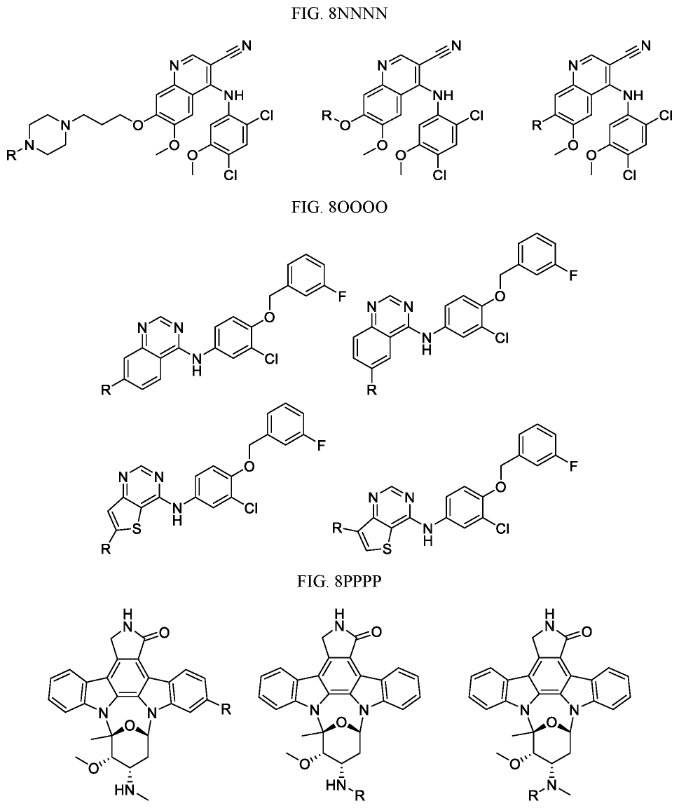

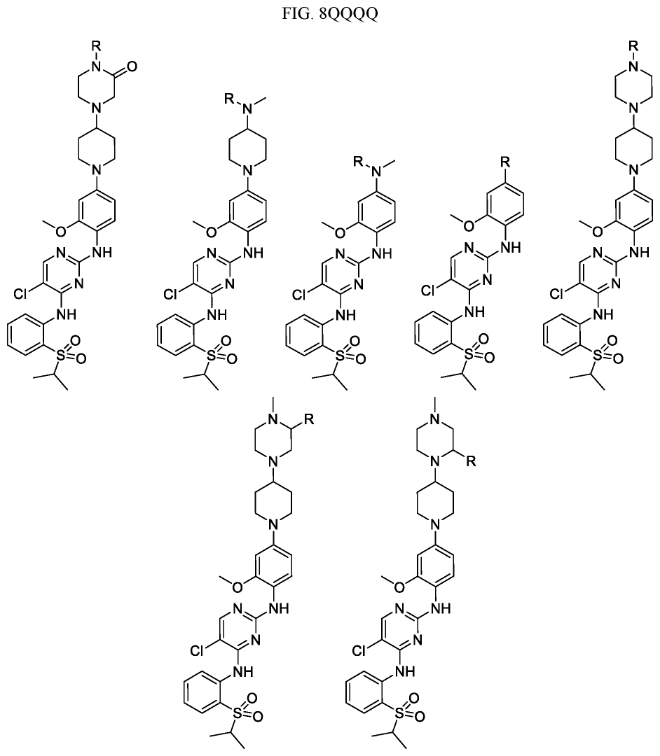

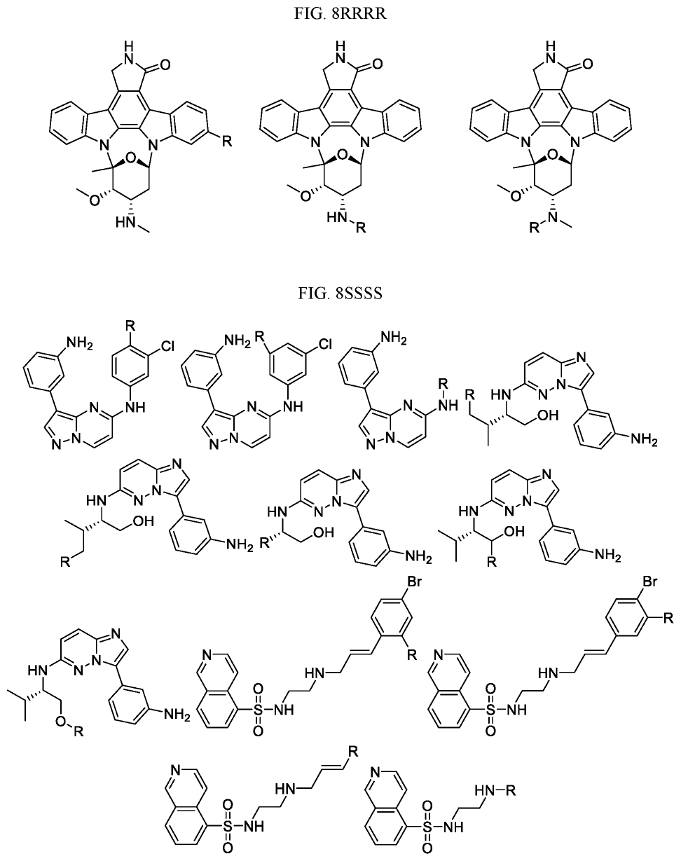

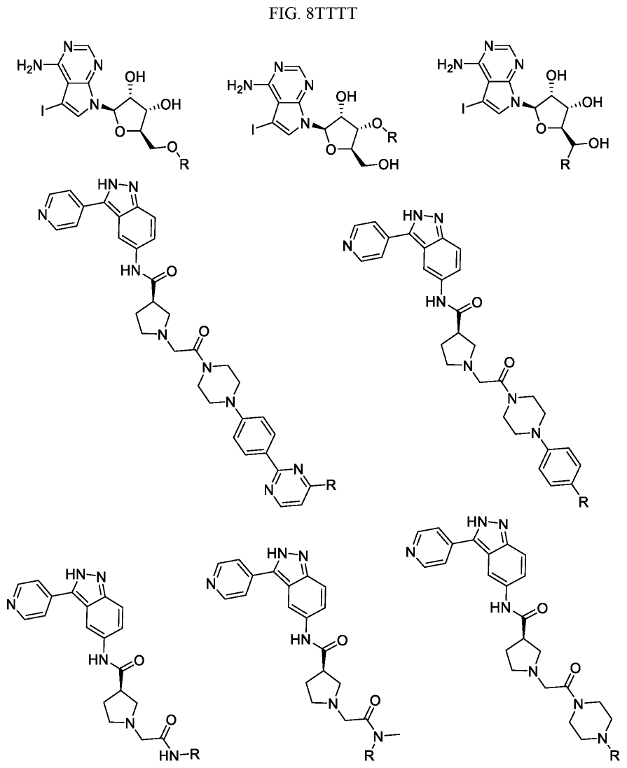

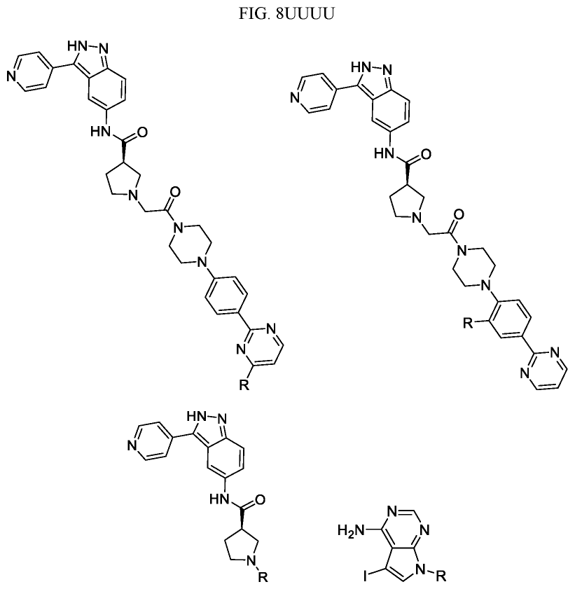

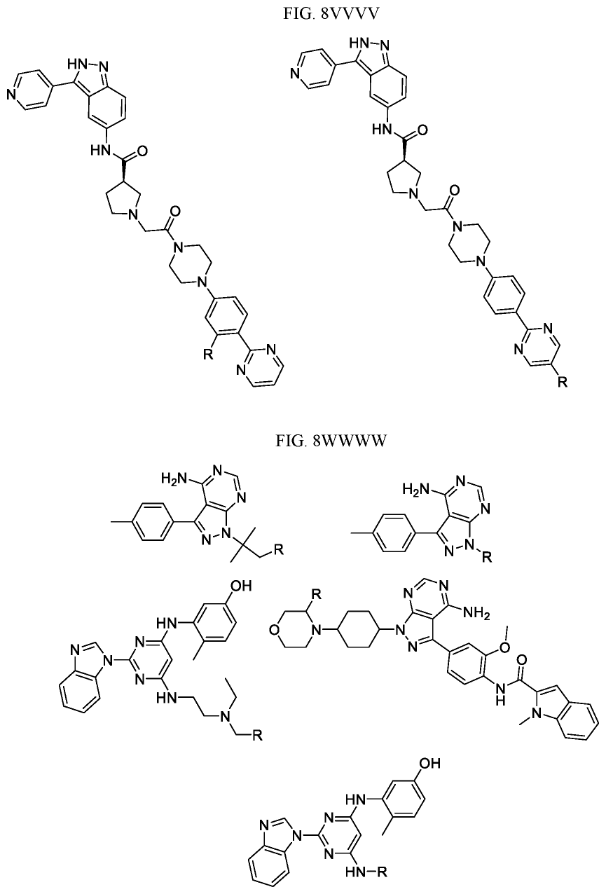

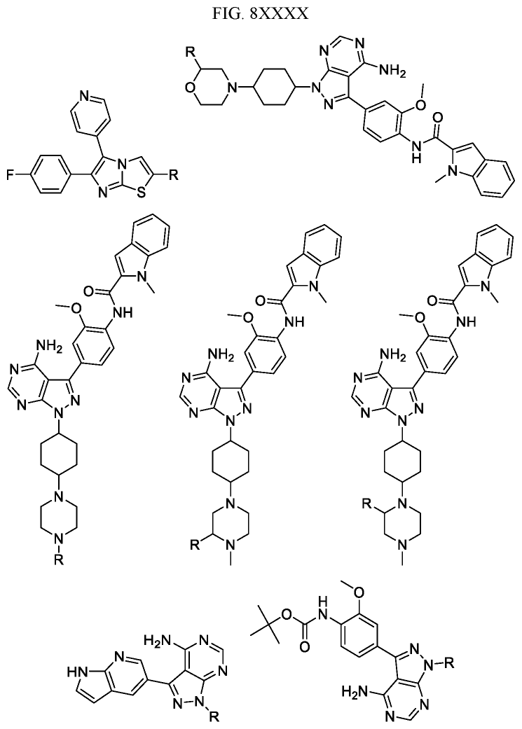

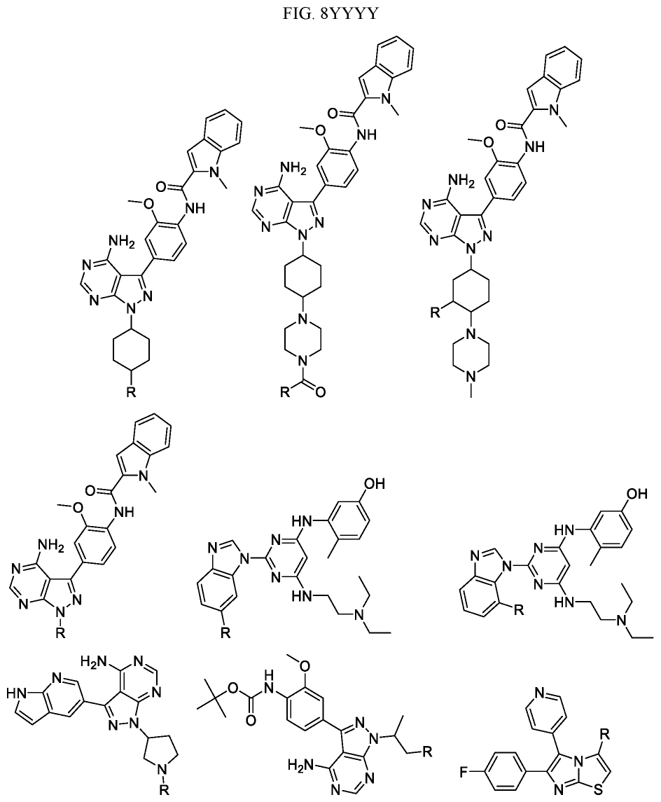

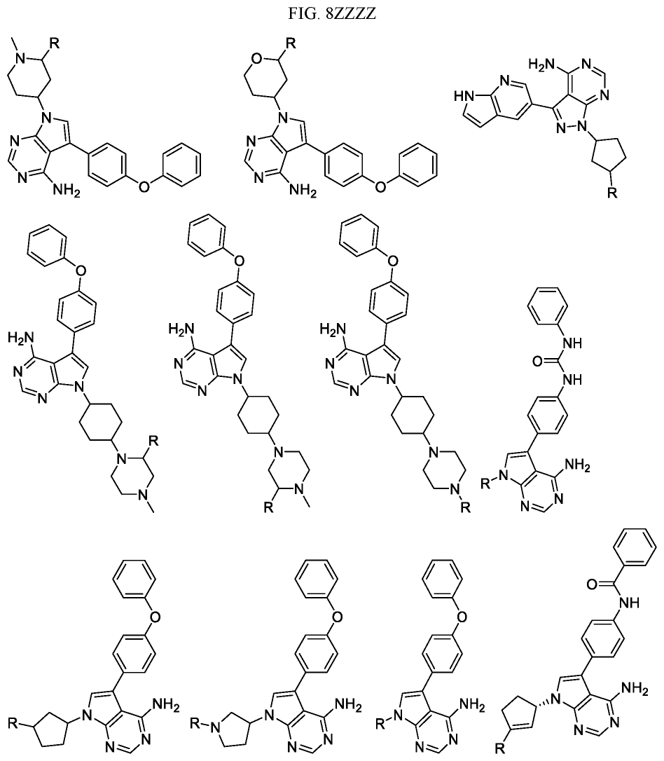

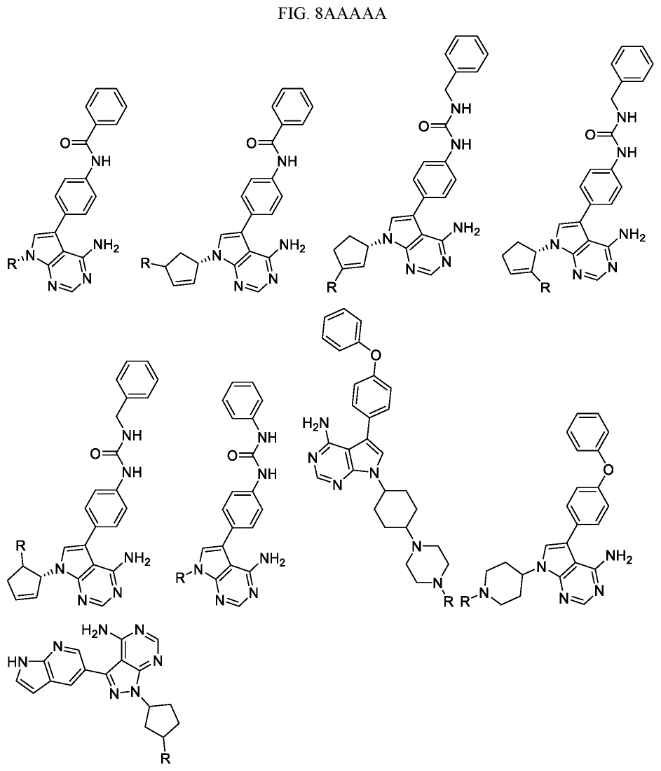

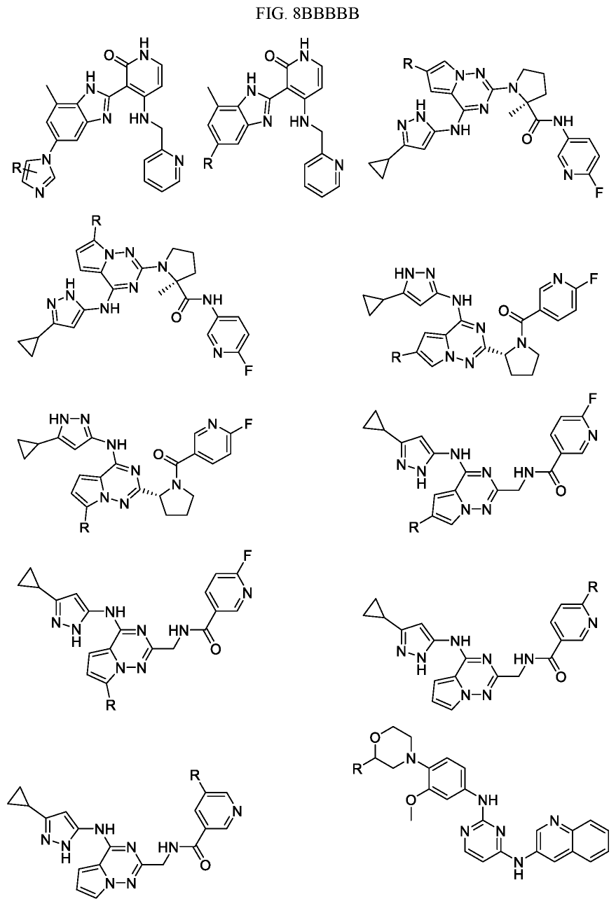

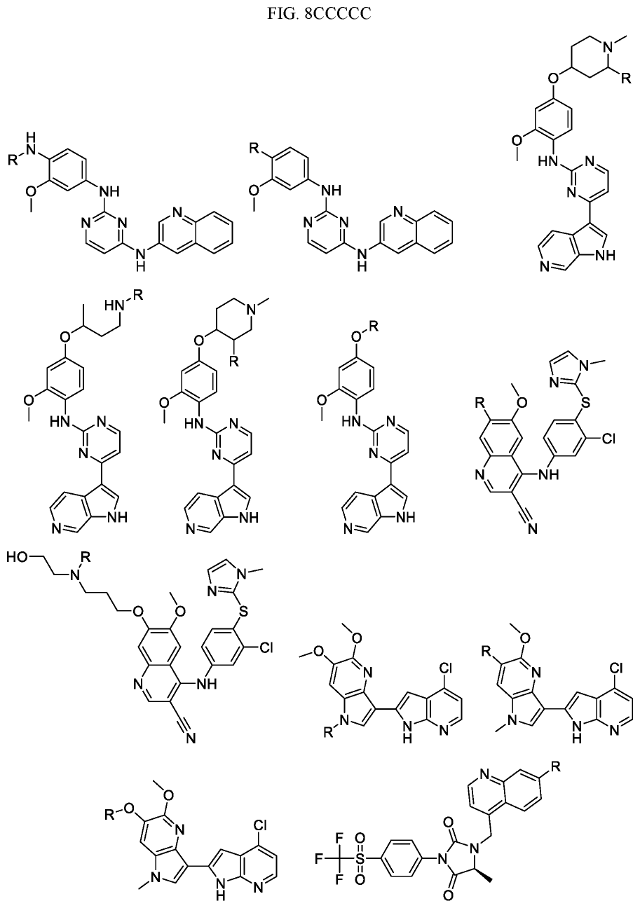

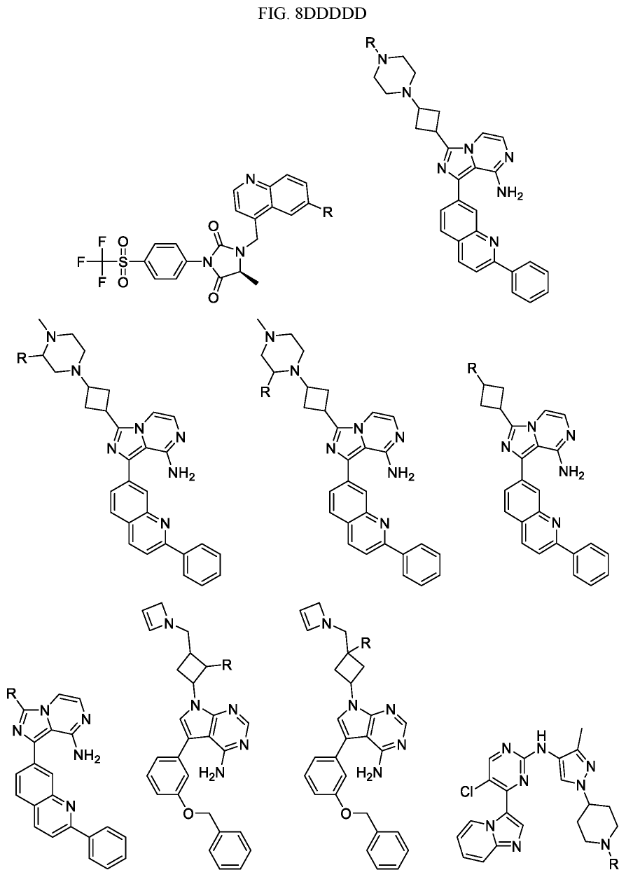

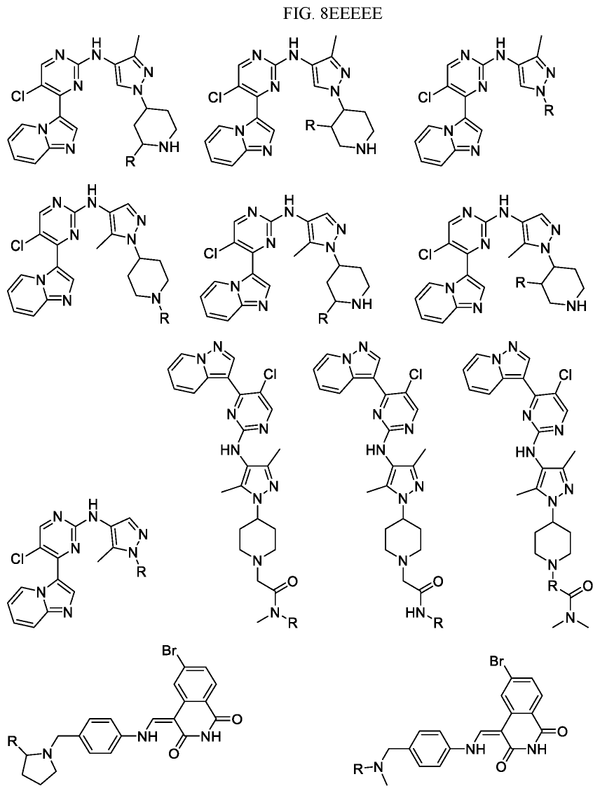

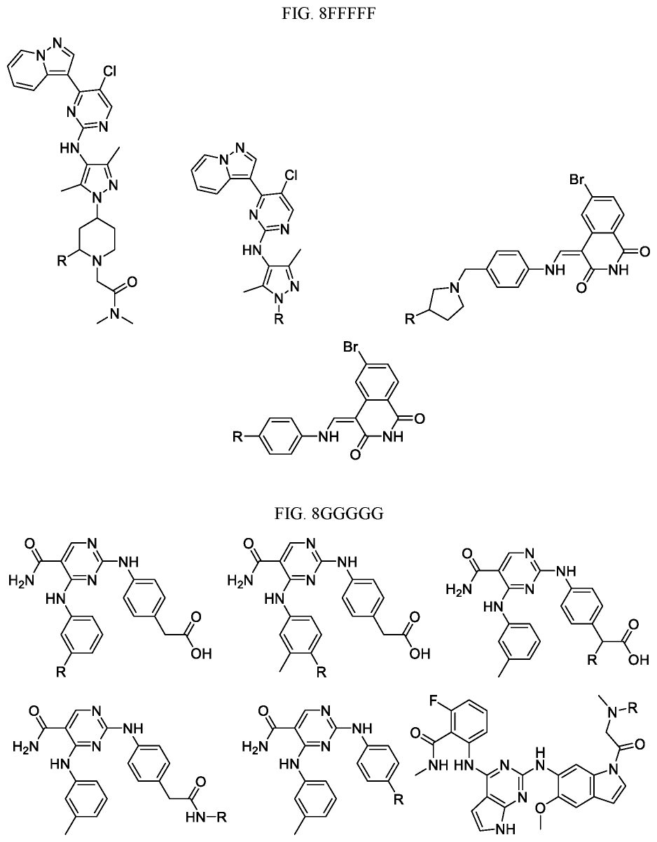

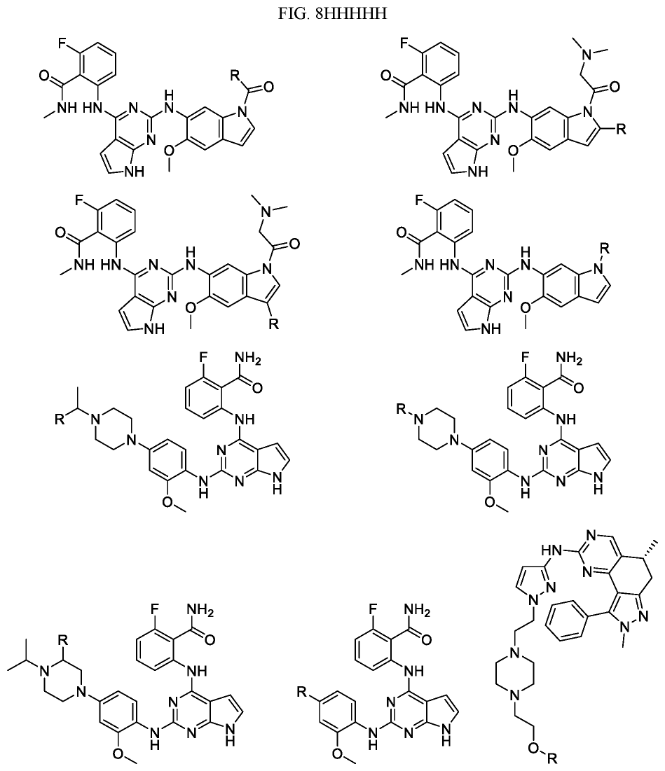

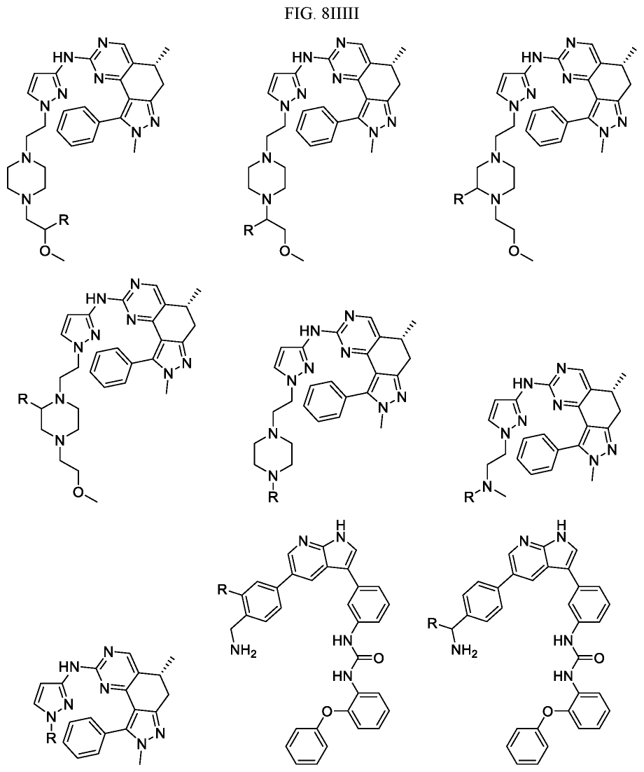

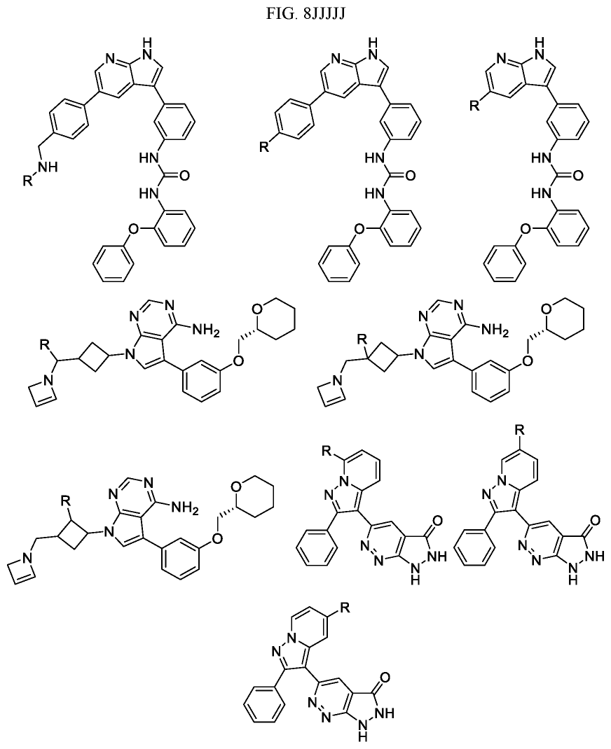

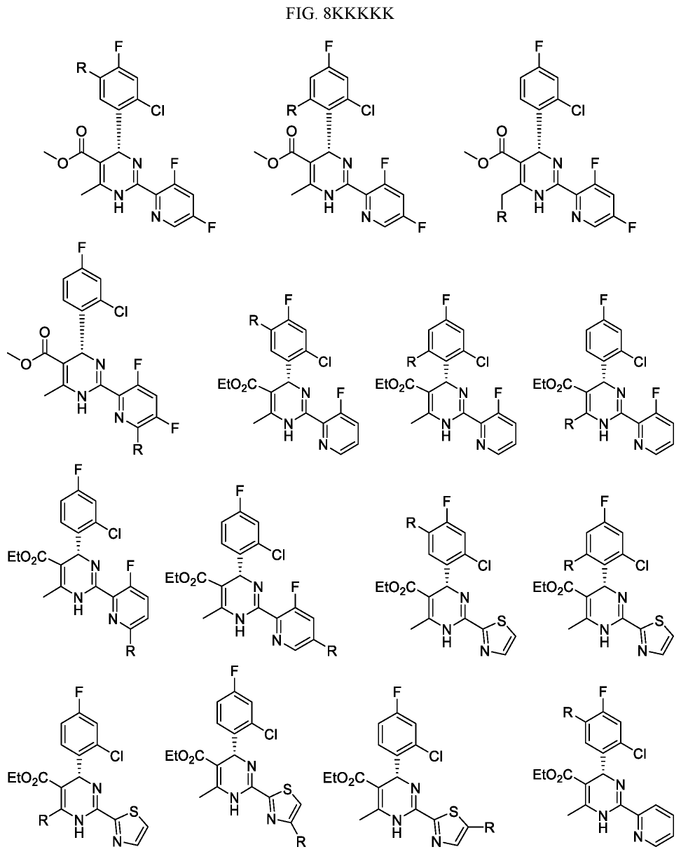

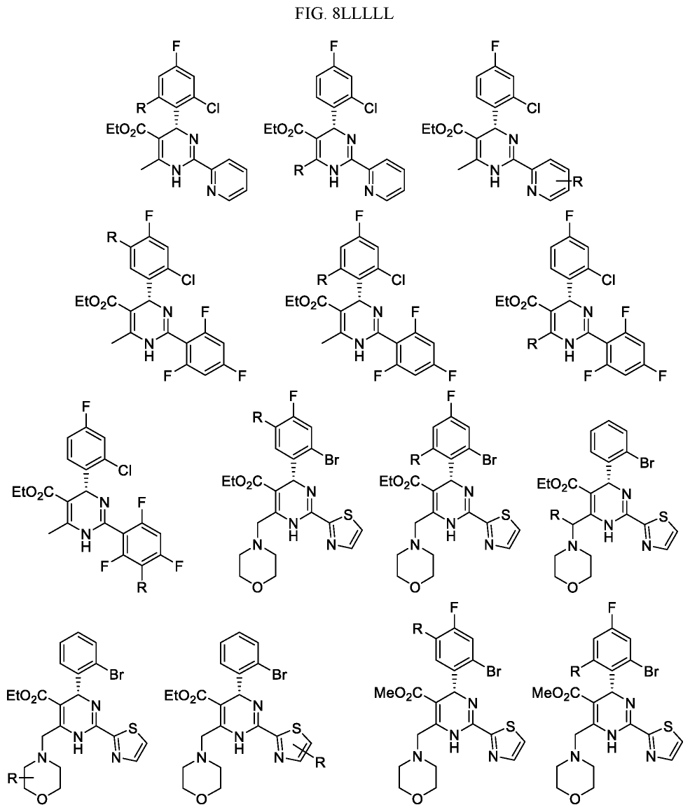

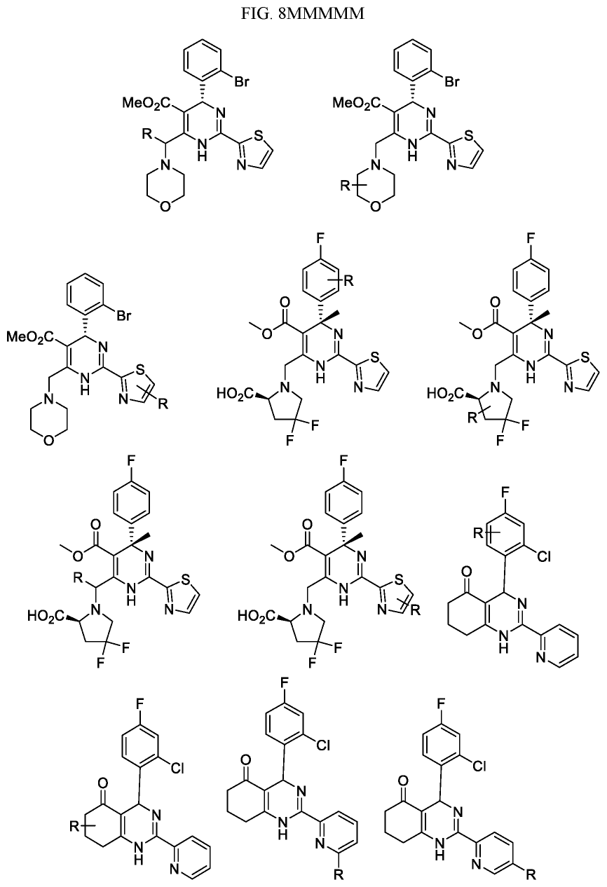

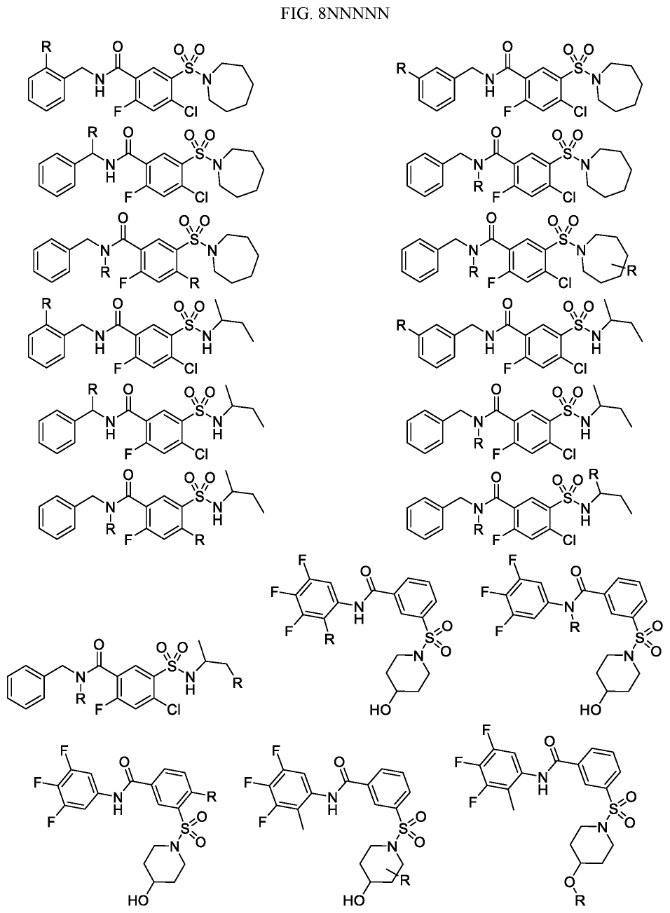

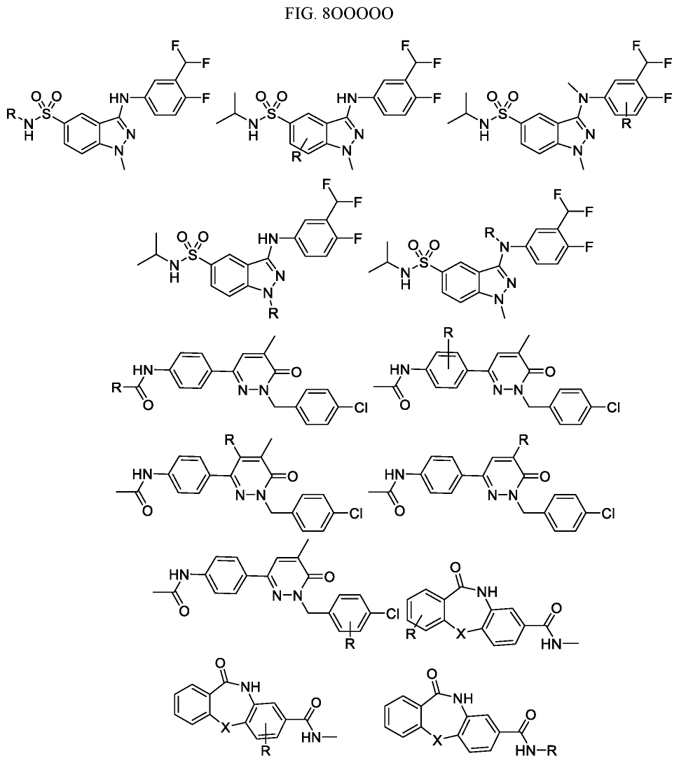

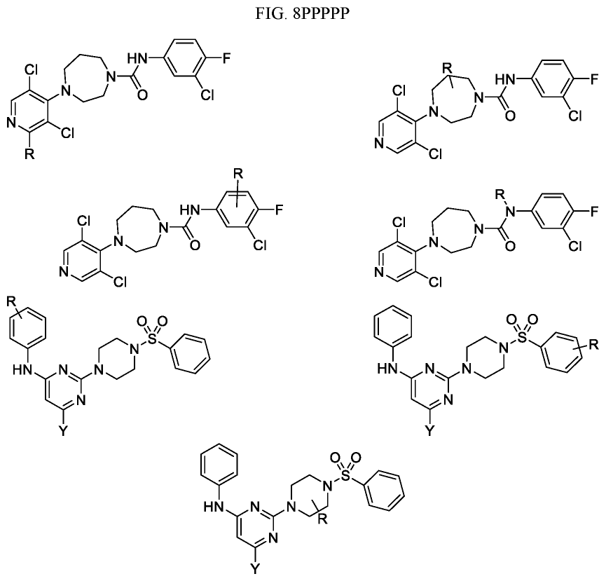

FIG. 8WW-8XX provide non-limiting examples of PAK1 Targeting Ligands wherein R represents exemplary points at which the spacer is attached. For additional examples and related ligands, see, Rudolph, J. et al. “Chemically Diverse Group I p21-Activated Kinase (PAK) Inhibitors Impart Acute Cardiovascular Toxicity with a Narrow Therapeutic Window.” J. Med. Chem. 59, 5520-5541 (2016) and Karpov A S, et al. ACS Med Chem Lett. 22; 6(7):776-81 (2015).

FIG. 8YY provides non-limiting examples of PAK4 Targeting Ligands wherein R represents exemplary points at which the spacer is attached. For additional examples and related ligands, see, Staben S T, et al. J. Med. Chem. 13; 57(3):1033-45 (2014) and Guo, C. et al. “Discovery of pyrroloaminopyrazoles as novel PAK inhibitors” J. Med. Chem. 55, 4728-4739 (2012).

FIG. 8ZZ-8AAA provide non-limiting examples of IDO Targeting Ligands wherein R represents exemplary points at which the spacer is attached. For additional examples and related ligands, see, Yue, E. W.; et al. “Discovery of potent competitive inhibitors of indoleamine 2,3-dioxygenase with in vivo pharmacodynamic activity and efficacy in a mouse melanoma model.” J. Med. Chem. 52, 7364-7367 (2009); Tojo, S.; et al. “Crystal structures and structure, and activity relationships of imidazothiazole derivatives as IDO1 inhibitors.” ACS Med. Chem. Lett. 5, 1119-1123 (2014); Mautino, M. R. et al. “NLG919, a novel indoleamine-2,3-dioxygenase (IDO)-pathway inhibitor drug candidate for cancer therapy” Abstract 491, AACR 104th Annual Meeting 2013; Apr. 6-10, 2013; Washington, DC; and, WO2012142237 titled “Fused imidazole derivatives useful as IDO inhibitors”.

FIG. 8BBB-8EEE provide non-limiting examples of ERK1 and ERK2 Targeting Ligands wherein R represents exemplary points at which the spacer is attached. For additional examples and related ligands, see, the crystal structures PDB 5K4I and 5K4J and related ligands described in Blake, J. F. et al. “Discovery of (S)-1-(1-(4-Chloro-3-fluorophenyl)-2-hydroxyethyl)-4-(2-((1-methyl-1H-pyrazol-5-yl)amino)pyrimidin-4-yl)pyridin-2(1H)-one (GDC-0994), an Extracellular Signal-Regulated Kinase 1/2 (ERK1/2) Inhibitor in Early Clinical Development” J. Med. Chem. 59: 5650-5660 (2016); the crystal structure PDB 5BVF and related ligands described in Bagdanoff, J. T. et al. “Tetrahydropyrrolo-diazepenones as inhibitors of ERK2 kinase” Bioorg. Med Chem. Lett. 25, 3788-3792 (2015); the crystal structure PDB 4QYY and related ligands described in Deng, Y. et al. “Discovery of Novel, Dual Mechanism ERK Inhibitors by Affinity Selection Screening of an Inactive Kinase” J. Med. Chem. 57: 8817-8826 (2014); the crystal structures PDB 5HD4 and 5HD7 and the related ligands described in Jha, S. et al. “Dissecting Therapeutic Resistance to ERK Inhibition” Mol. Cancer Ther. 15: 548-559 (2016); the crystal structure PDB 4XJ0 and related ligands described in Ren, L. et al. “Discovery of highly potent, selective, and efficacious small molecule inhibitors of ERK1/2.” J. Med. Chem. 58: 1976-1991 (2015); the crystal structures PDB 4ZZM, 4ZZN, 4ZZO and related ligands described in Ward, R. A. et al. “Structure-Guided Design of Highly Selective and Potent Covalent Inhibitors of Erk1/2.” J Med Chem. 58: 4790 (2015); Burrows, F. et al. “KO-947, a potent ERK inhibitor with robust preclinical single agent activity in MAPK pathway dysregulated tumors” Poster #5168, AACR National Meeting 2017; Bhagwat, S. V. et al. “Discovery of LY3214996, a selective and novel ERK1/2 inhibitor with potent antitumor activities in cancer models with MAPK pathway alterations.” AACR National Meeting 2017; the crystal structures PDB 3FHR and 3FXH and related ligands described in Cheng, R. et al. “High-resolution crystal structure of human Mapkap kinase 3 in complex with a high affinity ligand” Protein Sci. 19: 168-173 (2010); the crystal structures PDB 5NGU, 5NHF, 5NHH, 5NHJ, 5NHL, 5NHO, 5NHP, and 5NHV and related ligands described in Ward, R. A. et al. “Structure-Guided Discovery of Potent and Selective Inhibitors of ERK1/2 from a Modestly Active and Promiscuous Chemical Start Point.” J. Med. Chem. 60, 3438-3450 (2017); the crystal structures PDB 3SHE and 3R1N and related ligands described in Oubrie, A. et al. “Novel ATP competitive MK2 inhibitors with potent biochemical and cell-based activity throughout the series.” Bioorg. Med. Chem. Lett. 22: 613-618 (2012); “Structure-Guided Design of Potent and Selective Pyrimidylpyrrole Inhibitors of Extracellular Signal-Regulated Kinase (ERK) Using Conformational Control” J Med Chem 2009, 52(20), 6362; WO2015051341; “Discovery of a Potent and Selective Oral Inhibitor of ERK1/2 (AZD0364) That Is Efficacious in Both Monotherapy and Combination Therapy in Models of Non-small Cell Lung Cancer (NSCLC)” J Med Chem 2019, 62(24), 11004; and “ERK Inhibitor LY3214996 Targets ERK Pathway-Driven Cancers: A Therapeutic Approach Toward Precision Medicine” Mol Cancer Ther 2020, 19, 325.

FIG. 8FFF-8III provide non-limiting examples of ABL1 Targeting Ligands wherein R represents exemplary points at which the spacer is attached. For additional examples and related ligands, see, the crystal structure PDB 1fpu and 2e2b and related ligands described in Schindler, T, et al. “Structural mechanism for STI-571 inhibition of abelson tyrosine kinase”, Science 289: 1938-1942 (2000); and Horio, T. et al. “Structural factors contributing to the Abl/Lyn dual inhibitory activity of 3-substituted benzamide derivatives”, Bioorg. Med. Chem. Lett. 17: 2712-2717 (2007); the crystal structures PDB 2hzn and 2hiw and related ligands described in Cowan-Jacob, S. W. et al. “Structural biology contributions to the discovery of drugs to treat chronic myelogenous leukemia”, Acta Crystallog. Sect. D 63: 80-93 (2007) and Okram, B. et al. “A general strategy for creating”, Chem. Biol. 13: 779-786 (2006); the crystal structure PDB 3cs9 and related ligands described in Weisberg, E. et al. “Characterization of AMN107, a selective inhibitor of native and mutant Bcr-Abl”, Cancer Cell 7: 129-14 (2005); the crystal structure PDB 3ik3 and related ligands described in O'Hare, T. et al. “AP24534, a pan-BCR-ABL inhibitor for chronic myeloid leukemia, potently inhibits the T315I mutant and overcomes mutation-based resistance”, Cancer Cell 16: 401-412 (2009); the crystal structure PDB 3mss and related ligands described in Jahnke, W. et al. “Binding or bending: distinction of allosteric Abl kinase agonists from antagonists by an NMR-based conformational assay”, J. Am. Chem. Soc. 132: 7043-7048 (2010); the crystal structure PDB 3oy3 and related ligands described in Zhou, T. et al. “Structural Mechanism of the Pan-BCR-ABL Inhibitor Ponatinib (AP24534): Lessons for Overcoming Kinase Inhibitor Resistance”, Chem. Biol. Drug Des. 77: 1-11 (2011); the crystal structures PDB 3qri and 3qrk and related ligands described in Chan, W. W. et al. “Conformational Control Inhibition of the BCR-ABL1 Tyrosine Kinase, Including the Gatekeeper T315I Mutant, by the Switch-Control Inhibitor DCC-2036”, Cancer Cell 19: 556-568 (2011); the crystal structure PDB 5hu9 and 2f4j and related ligands described in Liu, F. et al. “Discovery and characterization of a novel potent type II native and mutant BCR-ABL inhibitor (CHMFL-074) for Chronic Myeloid Leukemia (CML)”, Oncotarget 7: 45562-45574 (2016) and Young, M. A. et al. “Structure of the kinase domain of an imatinib-resistant Abl mutant in complex with the Aurora kinase inhibitor VX-680”, Cancer Res. 66: 1007-1014 (2006); the crystal structure PDB 2gqg and 2qoh and related ligands described in Tokarski, J. S. et al. “The Structure of Dasatinib (BMS-354825) Bound to Activated ABL Kinase Domain Elucidates Its Inhibitory Activity against Imatinib-Resistant ABL Mutants”, Cancer Res. 66: 5790-5797 (2006); and Zhou, T. et al. “Crystal Structure of the T315I Mutant of Abl Kinase”, Chem. Biol. DrugDes. 70: 171-181 (2007); the crystal structure PDB 2gqg and 2qoh and related ligands described in Tokarski, J. S. et al. “The Structure of Dasatinib (BMS-354825) Bound to Activated ABL Kinase Domain Elucidates Its Inhibitory Activity against Imatinib-Resistant ABL Mutants”, Cancer Res. 66: 5790-5797 (2006) and Zhou, T. et al. “Crystal Structure of the T315I Mutant of Abl Kinase”, Chem. Biol. Drug Des. 70: 171-181 (2007); the crystal structure PDB 2gqg and 2qoh and related ligands described in Tokarski, J. S. et al. “The Structure of Dasatinib (BMS-354825) Bound to Activated ABL Kinase Domain Elucidates Its Inhibitory Activity against Imatinib-Resistant ABL Mutants”, Cancer Res. 66: 5790-5797 (2006) and Zhou, T. et al. “Crystal Structure of the T315I Mutant of Abl Kinase”, Chem. Biol. DrugDes. 70: 171-181(2007); the crystal structures PDB 3dk3 and 3dk8 and related ligands described in Berkholz, D. S. et al. “Catalytic cycle of human glutathione reductase near 1 A resolution” J. Mol. Biol. 382: 371-384 (2008); the crystal structure PDB 3ue4 and related ligands described in Levinson, N. M. et al. “Structural and spectroscopic analysis of the kinase inhibitor bosutinib and an isomer of bosutinib binding to the abl tyrosine kinase domain”, Plos One 7: e29828-e29828 (2012); the crystal structure PDB 4cy8 and related ligands described in Jensen, C. N. et al. “Structures of the Apo and Fad-Bound Forms of 2-Hydroxybiphenyl 3-Monooxygenase (Hbpa) Locate Activity Hotspots Identified by Using Directed Evolution”, Chembiochem 16: 968 (2015); the crystal structure PDB 2hz0 and related ligands described in Cowan-Jacob, S. W. et al. “Structural biology contributions to the discovery of drugs to treat chronic myelogenous leukaemia”, Acta Crystallogr D Biol Crystallogr. 63(Pt 1):80-93 (2007); the crystal structure PDB 3pyy and related ligands described in Yang, J. et al. “Discovery and Characterization of a Cell-Permeable, Small-Molecule c-Abl Kinase Activator that Binds to the Myristoyl Binding Site”, Chem. Biol. 18: 177-186 (2011); and, the crystal structure PDB 5k5v and related ligands described in Kim, M. K., et al. “Structural basis for dual specificity of yeast N-terminal amidase in the N-end rule pathway”, Proc. Natl. Acad Sci. U.S.A. 113: 12438-12443 (2016).

FIG. 8JJJ provide non-limiting examples of ABL2 Targeting Ligands wherein R represents exemplary points at which the spacer is attached. For additional examples and related ligands, see, the crystal structure PDB 2xyn and related ligands described in Salah, E. et al. “Crystal Structures of Abl-Related Gene (Abl2) in Complex with Imatinib, Tozasertib (Vx-680), and a Type I Inhibitor of the Triazole Carbothioamide Class”, J. Med. Chem. 54: 2359 (2011); the crystal structure PDB 4xli and related ligands described in Ha, B. H. et al. “Structure of the ABL2/ARG kinase in complex with dasatinib” Acta Crystallogr. Sect. F 71: 443-448 (2015); and the crystal structure PDB 3gvu and related ligands described in Salah, E. et al. “The crystal structure of human ABL2 in complex with Gleevec”, to be published.

FIG. 8KKK-8MMM provide non-limiting examples of AKT1 Targeting Ligands wherein R represents exemplary points at which the spacer is attached. For additional examples and related ligands, see, Lippa, B. et al. “Synthesis and structure based optimization of novel Akt inhibitors Bioorg. Med. Chem. Lett. 18: 3359-3363 (2008); Freeman-Cook, K. D. et al. “Design of selective, ATP-competitive inhibitors of Akt”, J. Med. Chem. 53: 4615-4622 (2010); Blake, J. F. et al “Discovery of pyrrolopyrimidine inhibitors of Akt”, Bioorg. Med. Chem. Lett. 20: 5607-5612 (2010); Kallan, N.C. et al. “Discovery and SAR of spirochromane Akt inhibitors”, Bioorg. Med Chem. Lett. 21: 2410-2414 (2011); Lin, K “An ATP-Site On-Off Switch That Restricts Phosphatase Accessibility of Akt”, Sci. Signal. 5: ra37-ra37 (2012); Addie, M. et al. “Discovery of 4-Amino-N-[(1S)-1-(4-chlorophenyl)-3-hydroxypropyl]-1-(7H-pyrrolo[2,3-d]pyrimidin-4-yl)piperidine-4-carboxamide (AZD5363), an Orally Bioavailable, Potent Inhibitor of Akt Kinases”, J. Med. Chem. 56: 2059-2073 (2013); Wu, W. I., et al. “Crystal structure of human AKT 1 with an allosteric inhibitor reveals a new mode of kinase inhibition. Plos One 5: 12913-12913 (2010); Ashwell, M. A. et al. “Discovery and optimization of a series of 3-(3-phenyl-3H-imidazo[4,5-b]pyridin-2-yl)pyridin-2-amines: orally bioavailable, selective, and potent ATP-independent Akt inhibitors”, J. Med. Chem. 55: 5291-5310 (2012); and, Lapierre, J. M. et al. “Discovery of 3-(3-(4-(1-Aminocyclobutyl)phenyl)-5-phenyl-3H-imidazo[4,5-b]pyridin-2-yl)pyridin-2-amine (ARQ 092): An Orally Bioavailable, Selective, and Potent Allosteric AKT Inhibitor”, J. Med. Chem. 59: 6455-6469 (2016).

FIG. 8NNN-8OOO provide non-limiting examples of AKT2 Targeting Ligands wherein R represents exemplary points at which the spacer is attached. For additional examples and related ligands, see, the crystal structured PDB 2jdo and 2jdr and related ligands described in Davies, T. G. et al. “A Structural Comparison of Inhibitor Binding to Pkb, Pka and Pka-Pkb Chimera”, J. Mol. Biol. 367: 882 (2007); the crystal structure PDB 2uw9 and related ligands described in Saxty, G. et al “Identification of Inhibitors of Protein Kinase B Using Fragment-Based Lead Discovery”, J. Med. Chem. 50: 2293-2296 (2007); the crystal structure PDB 2x39 and 2xh5 and related ligands described in Mchardy, T. et al. “Discovery of 4-Amino-1-(7H-Pyrrolo[2,3-D]Pyrimidin-4-Yl)Piperidine-4-Carboxamides as Selective, Orally Active Inhibitors of Protein Kinase B (Akt)”, J. Med. Chem. 53: 2239d (2010); the crystal structure PDB 3d03 and related ligands described in Hadler, K. S. et al. “Substrate-promoted formation of a catalytically competent binuclear center and regulation of reactivity in a glycerophosphodiesterase from Enterobacter aerogenes’, J. Am. Chem. Soc. 130: 14129-14138 (2008); and, the crystal structures PDB 3e87, 3e8d and 3e88 and related ligands described in Rouse, M. B. et al. “Aminofurazans as potent inhibitors of AKT kinase” Bioorg. Med. Chem. Lett. 19: 1508-1511 (2009).

FIG. 8PPP provides non-limiting examples of BMX Targeting Ligands wherein R represents exemplary points at which the spacer is attached. For additional examples and related ligands, see, the crystal structures PDB 3sxr and 3sxr and related ligands described in Muckelbauer, J. et al. “X-ray crystal structure of bone marrow kinase in the x chromosome: a Tec family kinase”, Chem. Biol. Drug Des. 78: 739-748 (2011).

FIG. 8QQQ-8SSS provide non-limiting examples of CSF1R Targeting Ligands wherein R represents exemplary points at which the spacer is attached. For additional examples and related ligands, see, the crystal structures PDB 2i0v and 2i1m and related ligands described in Schubert, C. et al. “Crystal structure of the tyrosine kinase domain of colony-stimulating factor-1 receptor (cFMS) in complex with two inhibitors”, J Biol. Chem. 282: 4094-4101 (2007); the crystal structure PDB 3bea and related ligands described in Huang, H. et al. “Design and synthesis of a pyrido[2,3-d]pyrimidin-5-one class of anti-inflammatory FMS inhibitors”, Bioorg. Med. Chem. Lett. 18: 2355-2361 (2008); the crystal structure PDB 3dpk and related ligands described in M. T., McKay, D. B. Overgaard, “Structure of the Elastase of Pseudomonas aeruginosa Complexed with Phosphoramidon”, to be published; the crystal structures PDB 3krj and 3krl and related ligands described in Illig, C. R. et al. “Optimization of a Potent Class of Arylamide Colony-Stimulating Factor-1 Receptor Inhibitors Leading to Anti-inflammatory Clinical Candidate 4-Cyano-N-[2-(1-cyclohexen-1-yl)-4-[1-[(dimethylamino)acetyl]-4-piperidinyl]phenyl]-1H-imidazole-2-carboxamide (JNJ-28312141”, J. Med. Chem. 54: 7860-7883 (2011); the crystal structure PDB 4r7h and related ligands described in Tap, W. D. et al. “Structure-Guided Blockade of CSF1R Kinase in Tenosynovial Giant-Cell Tumor, N Engl J Med 373: 428-437 (2015); the crystal structure PDB 31cd and 31coa and related ligands described in Meyers, M. J. et al. “Structure-based drug design enables conversion of a DFG-in binding CSF-1R kinase inhibitor to a DFG-out binding mod”, Bioorg. Med. Chem. Lett. 20: 1543-1547 (2010); the crystal structure PDB 4hw7 and related ligands described in Zhang, C. et al. “Design and pharmacology of a highly specific dual FMS and KIT kinase inhibitor”, Proc. Natl. Acad Sci. USA 110: 5689-5694 (2013); and, the crystal structure PDB 4r7i and related ligands described in Tap, W. D. et al. “Structure-Guided Blockade of CSF1R Kinase in Tenosynovial Giant-Cell Tumor”, N Engl J Med. 373: 428-437 (2015).

FIG. 8TTT provides non-limiting examples of CSK Targeting Ligands wherein R represents exemplary points at which the spacer is attached. For additional examples and related ligands, see, Levinson, N. M. et al. “Structural basis for the recognition of c-Src by its inactivator Csk”, Cell 134: 124-134 (2008).

FIG. 8UUU-8YYY provide non-limiting examples of DDR1 Targeting Ligands wherein R represents exemplary points at which the spacer is attached. For additional examples and related ligands, see, the crystal structures PDB 3zos and 4bkj and related ligands described in Canning, P. et al. “Structural Mechanisms Determining Inhibition of the Collagen Receptor Ddrl by Selective and Multi-Targeted Type II Kinase Inhibitors”, J. Mol. Biol. 426: 2457 (2014); the crystal structure PDB 4ckr and related ligands described in Kim, H. et al. “Discovery of a Potent and Selective Ddrl Receptor Tyrosine Kinase Inhibitor”, ACS Chem. Biol. 8: 2145 (2013); the crystal structure PDB 5bvk, 5bvn and 5bvw and related ligands described in Murray, C. W et al. “Fragment-Based Discovery of Potent and Selective DDR1/2 Inhibitors”, ACS Med. Chem. Lett. 6: 798-803 (2015); the crystal structure PDB 5fdp and related ligands described in Wang, Z. et al. “Structure-Based Design of Tetrahydroisoquinoline-7-carboxamides as Selective Discoidin Domain Receptor 1 (DDR1) Inhibitors”, J. Med. Chem. 59: 5911-5916 (2016); and, the crystal structure PDB 5fdx and related ligands described in Bartual, S. G. et al. “Structure of DDR1 receptor tyrosine kinase in complex with D2164 inhibitor at 2.65 Angstroms resolution”, to be published.

FIG. 8ZZZ-8CCCC provide non-limiting examples of EPHA2 Targeting Ligands wherein R represents exemplary points at which the spacer is attached. For additional examples and related ligands, see, the crystal structures PDB 5i9x, 5i9y, 5ia0 and 5ia1 and related ligands described in Heinzlmeir, S. et al. “Chemical Proteomics and Structural Biology Define EPHA2 Inhibition by Clinical Kinase Drug”, ACS Chem. Biol. 11: 3400-3411 (2016); the crystal structure PDB 5i9z and related ligands described in Heinzlmeir, S. et al. “Crystal Structure of Ephrin A2 (EphA2) Receptor Protein Kinase with danusertib (PHA739358)”, ACS Chem Biol 11 3400-3411 (2016); and, the crystal structures PDB 5ia2, 5ia3, 5ia4, and 5ia5 and related ligands described in Heinzlmeir, S. et al. “Chemical Proteomics and Structural Biology Define EPHA2 Inhibition by Clinical Kinase Drug”, ACS Chem. Biol. 11: 3400-3411 (2016).

FIG. 8DDDD-8FFFF provide non-limiting examples of EPHA3 Targeting Ligands wherein R represents exemplary points at which the spacer is attached. For additional examples and related ligands, see, the crystal structure PDB 4g2f and related ligands described in Zhao, H. et al. “Discovery of a novel chemotype of tyrosine kinase inhibitors by fragment-based docking and molecular dynamics”, ACS Med. Chem. Lett. 3: 834-838 (2012); the crystal structure PDB 4gk2 and 4gk3 and related ligands described in Lafleur, K. et al. “Optimization of Inhibitors of the Tyrosine Kinase EphB4. 2. Cellular Potency Improvement and Binding Mode Validation by X-ray Crystallography”, J. Med. Chem. 56: 84-96 (2013); the crystal structure PDB 4gk3 and related ligands described in Lafleur, K. et al. “Optimization of Inhibitors of the Tyrosine Kinase EphB4. 2. Cellular Potency Improvement and Binding Mode Validation by X-ray Crystallography”, J. Med. Chem. 56: 84-96 (2013); the crystal structure PDB 4p4c and 4p5q and related ligands described in Unzue, A. et al. “Pyrrolo[3,2-b]quinoxaline Derivatives as Types I1/2 and II Eph Tyrosine Kinase Inhibitors: Structure-Based Design, Synthesis, and in Vivo Validation”, J. Med. Chem. 57: 6834-6844 (2014); the crystal structure PDB 4p5z and related ligands described in Unzue, A. et al. “Pyrrolo[3,2-b]quinoxaline Derivatives as Types I1/2 and II Eph Tyrosine Kinase Inhibitors: Structure-Based Design, Synthesis, and in Vivo Validation”, J. Med. Chem. 57: 6834-6844 (2014); the crystal structure PDB 4twn and related ligands described in Dong, J. et al. “Structural Analysis of the Binding of Type I, I1/2, and II Inhibitors to Eph Tyrosine Kinases”, ACS Med. Chem. Lett. 6: 79-83 (2015); the crystal structure PDB 3dzq and related ligands described in Walker, J. R. “Kinase Domain of Human Ephrin Type-A Receptor 3 (Epha3) in Complex with ALW-II-38-3”, to be published.

FIG. 8GGGG provides non-limiting examples of EPHA4 Targeting Ligands wherein R represents exemplary points at which the spacer is attached. For additional examples and related ligands, see, the crystal structure PDB 2y60 and related ligands described in Clifton, I. J. et al. “The Crystal Structure of Isopenicillin N Synthase with Delta((L)-Alpha-Aminoadipoyl)-(L)-Cysteinyl-(D)-Methionine Reveals Thioether Coordination to Iron”, Arch. Biochem. Biophys. 516: 103 (2011) and the crystal structure PDB 2xyu and related ligands described in Van Linden, O. P et al. “Fragment Based Lead Discovery of Small Molecule Inhibitors for the Epha4 Receptor Tyrosine Kinase”, Eur. J. Med. Chem. 47: 493 (2012).

FIG. 8HHHH provides non-limiting examples of EPHA7 Targeting Ligands wherein R represents exemplary points at which the spacer is attached. For additional examples and related ligands, see, the crystal structure PDB 3dko and related ligands described in Walker, J. R. et al. “Kinase domain of human ephrin type-a receptor 7 (epha7) in complex with ALW-II-49-7”, to be published.

FIG. 8IIII-8LLLL provide non-limiting examples of EPHB4 Targeting Ligands wherein R represents exemplary points at which the spacer is attached. For additional examples and related ligands, see, the crystal structure PDB 2vx1 and related ligands described in Bardelle, C. et al. “Inhibitors of the Tyrosine Kinase Ephb4. Part 2: Structure-Based Discovery and Optimization of 3,5-Bis Substituted Anilinopyrimidines”, Bioorg. Med. Chem. Lett. 18: 5717(2008); the crystal structure PDB 2x9f and related ligands described in Bardelle, C. et al. “Inhibitors of the Tyrosine Kinase Ephb4. Part 3: Identification of Non-Benzodioxole-Based Kinase Inhibitors”, Bioorg. Med Chem. Lett. 20: 6242-6245 (2010); the crystal structure PDB 2xvd and related ligands described in Barlaam, B. et al. “Inhibitors of the Tyrosine Kinase Ephb4. Part 4: Discovery and Optimization of a Benzylic Alcohol Series”, Bioorg. Med. Chem. Lett. 21: 2207 (2011); the crystal structure PDB 3zew and related ligands described in Overman, R. C. et al. “Completing the Structural Family Portrait of the Human Ephb Tyrosine Kinase Domains”, Protein Sci. 23: 627 (2014); the crystal structure PDB 4aw5 and related ligands described in Kim, M. H. et al. “The Design, Synthesis, and Biological Evaluation of Potent Receptor Tyrosine Kinase Inhibitors”, Bioorg. Med. Chem. Lett. 22: 4979 (2012); the crystal structure PDB 4bb4 and related ligands described in Vasbinder, M. M. et al. “Discovery and Optimization of a Novel Series of Potent Mutant B-Raf V600E Selective Kinase Inhibitors” J. Med. Chem. 56: 1996.”, (2013); the crystal structures PDB 2vwu, 2vwv and 2vww and related ligands described in Bardelle, C. et al “Inhibitors of the Tyrosine Kinase Ephb4. Part 1: Structure-Based Design and Optimization of a Series of 2,4-Bis-Anilinopyrimidines”, Bioorg. Med. Chem. Lett. 18: 2776-2780 (2008); the crystal structures PDB 2vwx, 2vwy, and 2vwz and related ligands described in Bardelle, C. et al. “Inhibitors of the Tyrosine Kinase Ephb4. Part 2: Structure-Based Discovery and Optimization of 3,5-Bis Substituted Anilinopyrimidines”, Bioorg. Med. Chem. Lett. 18: 5717 (2008); and, the crystal structure PDB 2vxo and related ligands described in Welin, M. et al. “Substrate Specificity and Oligomerization of Human Gmp Synthetas”, J. Mol. Biol. 425: 4323 (2013).

FIG. 8MMMM provides non-limiting examples of ERBB2 Targeting Ligands wherein R represents exemplary points at which the spacer is attached. For additional examples and related ligands, see, the crystal structure and related ligands described in Aertgeerts, K. et al “Structural Analysis of the Mechanism of Inhibition and Allosteric Activation of the Kinase Domain of HER2 Protein”, J. Biol. Chem. 286: 18756-18765 (2011) and the crystal structure and related ligands described in Ishikawa, T. et al. “Design and Synthesis of Novel Human Epidermal Growth Factor Receptor 2 (HER2)/Epidermal Growth Factor Receptor (EGFR) Dual Inhibitors Bearing a Pyrrolo[3,2-d]pyrimidine Scaffold” J. Med. Chem. 54: 8030-8050 (2011).

FIG. 8NNNN provides non-limiting examples of ERBB3 Targeting Ligands wherein R represents exemplary points at which the spacer is attached. For additional examples and related ligands, see, Littlefield, P. et al. “An ATP-Competitive Inhibitor Modulates the Allosteric Function of the HER3 Pseudokinase”, Chem. Biol. 21: 453-458 (2014).

FIG. 8OOOO provides non-limiting examples ERBB4 Targeting Ligands wherein R represents exemplary points at which the spacer is attached. For additional examples and related ligands, see, Qiu, C. et al. “Mechanism of Activation and Inhibition of the HER4/ErbB4 Kinase”, Structure 16: 460-467 (2008) and Wood, E. R. et al. “6-Ethynylthieno[3,2-d]- and 6-ethynylthieno[2,3-d]pyrimidin-4-anilines as tunable covalent modifiers of ErbB kinases”, Proc. Natl. Acad Sci. Usa 105: 2773-2778 (2008).

FIG. 8PPPP-8QQQQ provide non-limiting examples of FES Targeting Ligands wherein R represents exemplary points at which the spacer is attached. For additional examples and related ligands, see, Filippakopoulos, P. et al “Structural Coupling of SH2-Kinase Domains Links Fes and Abl Substrate Recognition and Kinase Activation.” Cell 134: 793-803 (2008) and Hellwig, S. et al. “Small-Molecule Inhibitors of the c-Fes Protein-Tyrosine Kinase”, Chem. Biol. 19: 529-540 (2012).

FIG. 8RRRR provides non-limiting examples of FYN Targeting Ligands wherein R represents exemplary points at which the spacer is attached. For additional examples and related ligands, see, Kinoshita, T. et. al. “Structure of human Fyn kinase domain complexed with staurosporine”, Biochem. Biophys. Res. Commun. 346: 840-844 (2006).

FIG. 8SSSS-8VVVV provide non-limiting examples of GSG2 (Haspin) Targeting Ligands wherein R represents exemplary points at which the spacer is attached. For additional examples and related ligands, see, the crystal structures PDB 3e7v, PDB 3f2n, 3fmd and related ligands described in Filippakopoulos, P. et al. “Crystal Structure of Human Haspin with a pyrazolo-pyrimidine ligand”, to be published; the crystal structure PDB 3iq7 and related ligands described in Eswaran, J. et al. “Structure and functional characterization of the atypical human kinase haspin”, Proc. Natl. Acad. Sci. USA 106: 20198-20203 (2009); and, the crystal structure PDB 4qtc and related ligands described in Chaikuad, A. et al. “A unique inhibitor binding site in ERK1/2 is associated with slow binding kinetics”, Nat. Chem. Biol. 10: 853-860 (2014).

FIG. 8WWWW-8AAAAA provide non-limiting examples of HCK Targeting Ligands wherein R represents exemplary points at which the spacer is attached. For additional examples and related ligands, see, the crystal structure PDB 1qcf and related ligands described in Schindler, T. et al. “Crystal structure of Hck in complex with a Src family-selective tyrosine kinase inhibitor”, Mol. Cell 3: 639-648 (1999); the crystal structure PDB 2c0i and 2c0t and related ligands described in Burchat, A. et al. “Discovery of A-770041, a Src-Family Selective Orally Active Lck Inhibitor that Prevents Organ Allograft Rejection”, Bioorg. Med. Chem. Lett. 16: 118 (2006); the crystal structure PDB 2hk5 and related ligands described in Sabat, M. et al. “The development of 2-benzimidazole substituted pyrimidine based inhibitors of lymphocyte specific kinase (Lck)”, Bioorg. Med. Chem. Lett. 16: 5973-5977 (2006); the crystal structures PDB 3vry, 3vs3, 3vs6, and 3vs7 and related ligands described in Saito, Y. et al. “A Pyrrolo-Pyrimidine Derivative Targets Human Primary AML Stem Cells in Vivo”, Sci Transl Med 5: 181ra52-181ra52 (2013); and, the crystal structure PDB 4lud and related ligands described in Parker, L. J. et al “Kinase crystal identification and ATP-competitive inhibitor screening using the fluorescent ligand SKF86002”, Acta Crystallogr., Sect. D 70: 392-404 (2014).

FIG. 8BBBBB-8FFFFF provide non-limiting examples of IGF1R Targeting Ligands wherein R represents exemplary points at which the spacer is attached. For additional examples and related ligands, see, the crystal structure PDB 2oj9 and related ligands described in Velaparthi, U. et al. “Discovery and initial SAR of 3-(1H-benzo[d]imidazol-2-yl)pyridin-2(1H)-ones as inhibitors of insulin-like growth factor 1-receptor (IGF-1R)”, Bioorg. Med. Chem. Lett. 17: 2317-2321 (2007); the crystal structure PDB 3i81 and related ligands described in Wittman, M. D. et al. “Discovery of a 2,4-disubstituted pyrrolo[1,2-f][1,2,4]triazine inhibitor (BMS-754807) of insulin-like growth factor receptor (IGF-1R) kinase in clinical development.”, J. Med. Chem. 52: 7360-7363 (2009); the crystal structure PDB 3nw5 and related ligands described in Sampognaro, A. J. et al. “Proline isosteres in a series of 2,4-disubstituted pyrrolo[1,2-f][1,2,4]triazine inhibitors of IGF-1R kinase and IR kinase”, Bioorg. Med. Chem. Lett. 20: 5027-5030 (2010); the crystal structure PDB 3qqu and related ligands described in Buchanan, J. L. et al. “Discovery of 2,4-bis-arylamino-1,3-pyrimidines as insulin-like growth factor-1 receptor (IGF-1R) inhibitors”, Bioorg. Med Chem. Lett. 21: 2394-2399 (2011); the crystal structure PDB 4d2r and related ligands described in Kettle, J. G. et al. “Discovery and Optimization of a Novel Series of DyrklB Kinase Inhibitors to Explore a Mek Resistance Hypothesis”. J. Med. Chem. 58: 2834 (2015); the crystal structure PDB 3fxq and related ligands described in Monferrer, D. et al. “Structural studies on the full-length LysR-type regulator TsaR from Comamonas testosteroni T-2 reveal a novel open conformation of the tetrameric LTTR fold”, Mol. Microbiol. 75: 1199-1214 (2010); the crystal structure PDB 5fxs and related ligands described in Degorce, S. et al. “Discovery of Azd9362, a Potent Selective Orally Bioavailable and Efficacious Novel Inhibitor of Igf-R1”, to be published; the crystal structure PDB 2zm3 and related ligands described in Mayer, S. C. et al. “Lead identification to generate isoquinolinedione inhibitors of insulin-like growth factor receptor (IGF-1R) for potential use in cancer treatment”, Bioorg. Med. Chem. Lett. 18: 3641-3645 (2008); the crystal structure PDB 3f5p and related ligands described in “Lead identification to generate 3-cyanoquinoline inhibitors of insulin-like growth factor receptor (IGF-1R) for potential use in cancer treatment” Bioorg. Med Chem. Lett. 19: 62-66 (2009); the crystal structure PDB 31vp and related ligands described in Nemecek, C. et al. “Design of Potent IGF1-R Inhibitors Related to Bis-azaindoles” Chem. Biol. Drug Des. 76: 100-106 (2010); the crystal structure PDB 3o23 and related ligands described in Lesuisse, D. et al. “Discovery of the first non-ATP competitive IGF-1R kinase inhibitors: Advantages in comparison with competitive inhibitors”, Bioorg. Med. Chem. Lett. 21: 2224-2228 (2011); the crystal structure PDB 3d94 and related ligands described in Wu, J. et al. “Small-molecule inhibition and activation-loop trans-phosphorylation of the IGF1 receptor”, Embo J. 27: 1985-1994 (2008); and, the crystal structure PDB 5hzn and related ligands described in Stauffer, F. et al. “Identification of a 5-[3-phenyl-(2-cyclic-ether)-methylether]-4-aminopyrrolo[2,3-d]pyrimidine series of IGF-1R inhibitors”, Bioorg. Med. Chem. Lett. 26: 2065-2067 (2016).

FIG. 8GGGGG-8JJJJJ provide non-limiting examples of INSR Targeting Ligands wherein R represents exemplary points at which the spacer is attached. For additional examples and related ligands, see, the crystal structure PDB 2z8c and related ligands described in Katayama, N. et al. “Identification of a key element for hydrogen-bonding patterns between protein kinases and their inhibitors”, Proteins 73: 795-801 (2008); the crystal structure PDB 3ekk and related ligands described in Chamberlain, S. D. et al. “Discovery of 4,6-bis-anilino-1H-pyrrolo[2,3-d]pyrimidines: Potent inhibitors of the IGF-1R receptor tyrosine kinase”, (2009) Bioorg. Med Chem. Lett. 19: 469-473; the crystal structure PDB 3ekn and related ligands described in Chamberlain, S. D. et al. “Optimization of 4,6-bis-anilino-1H-pyrrolo[2,3-d]pyrimidine IGF-1R tyrosine kinase inhibitors towards INK selectivity”, Bioorg. Med. Chem. Lett. 19: 360-364 (2009); the crystal structure PDB 5e1s and related ligands described in Sanderson, M. P. et al. “BI 885578, a Novel IGF1R/INSR Tyrosine Kinase Inhibitor with Pharmacokinetic Properties That Dissociate Antitumor Efficacy and Perturbation of Glucose Homeostasis” Mol. Cancer Ther. 14: 2762-2772”, (2015); the crystal structure PDB 3eta and related ligands described in Patnaik, S. et al. “Discovery of 3,5-disubstituted-1H-pyrrolo[2,3-b]pyridines as potent inhibitors of the insulin-like growth factor-1 receptor (IGF-1R) tyrosine kinase”, Bioorg. Med. Chem. Lett. 19: 3136-3140 (2009); the crystal structure PDB 5hhw and related ligands described in Stauffer, F. et al. “Identification of a 5-[3-phenyl-(2-cyclic-ether)-methylether]-4-aminopyrrolo[2,3-d]pyrimidine series of IGF-1R inhibitors”, Bioorg. Med. Chem. Lett. 26: 2065-2067 (2016); and, the crystal structure PDB 4ibm and related ligands described in Anastassiadis, T. et al. “A highly selective dual insulin receptor (IR)/insulin-like growth factor 1 receptor (IGF-1R) inhibitor derived from an extracellular signal-regulated kinase (ERK) inhibitor”, J. Biol. Chem. 288: 28068-28077 (2013).

FIG. 8KKKKK-8PPPPP provide non-limiting examples of HBV Targeting Ligands wherein R represents exemplary points at which the spacer is attached, Y is methyl or isopropyl, and X is N or C. For additional examples and related ligands, see, Weber, O.; et al. “Inhibition of human hepatitis B virus (HBV) by a novel non-nucleosidic compound in a transgenic mouse model.” Antiviral Res. 54, 69-78 (2002); Deres, K.; et al. “Inhibition of hepatitis B virus replication by drug-induced depletion of nucleocapsids.” Science, 299, 893-896 (2003); Stray, S. J.; Zlotnick, A. “BAY 41-4109 has multiple effects on Hepatitis B virus capsid assembly.” J. Mol. Recognit. 19, 542-548 (2006); Stray, S. J.; et al. “heteroaryldihydropyrimidine activates and can misdirect hepatitis B virus capsid assembly.” Proc. Natl. Acad. Sci. U.S.A., 102, 8138-8143 (2005); Guan, H.; et al. “The novel compound Z060228 inhibits assembly of the HBV capsid.” Life Sci. 133, 1-7 (2015); Wang, X. Y.; et al. “In vitro inhibition of HBV replication by a novel compound, GLS4, and its efficacy against adefovir-dipivoxil-resistant HBV mutations.” Antiviral Ther. 17, 793-803 (2012); Klumpp, K.; et al. “High-resolution crystal structure of a hepatitis B virus replication inhibitor bound to the viral core protein.” 112, 15196-15201 (2015); Qiu, Z.; et al. “Design and synthesis of orally bioavailable 4-methyl heteroaryldihydropyrimidine based hepatitis B virus (HBV) capsid inhibitors.” J. Med. Chem. 59, 7651-7666 (2016); Zhu, X.; et al. “2,4-Diaryl-4,6,7,8-tetrahydroquinazolin-5(1H)-one derivatives as anti-HBV agents targeting at capsid assembly.” Bioorg. Med. Chem. Lett. 20, 299-301 (2010); Campagna, M. R.; et al. “Sulfamoylbenzamide derivatives inhibit the assembly of hepatitis B virus nucleocapsids.” J. Virol. 87, 6931-6942 (2013); Campagna, M. R.; et al. “Sulfamoylbenzamide derivatives inhibit the assembly of hepatitis B virus nucleocapsids.” J. Virol. 87, 6931-6942 (2013); WO 2013096744 A1 titled “Hepatitis B antiviral agents”; WO 2015138895 titled “Hepatitis B core protein allosteric modulators”; Wang, Y. J.; et al. “A novel pyridazinone derivative inhibits hepatitis B virus replication by inducing genome-free capsid formation.” Antimicrob. Agents Chemother. 59, 7061-7072 (2015); WO 2014033167 titled “Fused bicyclic sulfamoyl derivatives for the treatment of hepatitis”; U.S. 20150132258 titled “Azepane derivatives and methods of treating hepatitis B infections”; and, WO 2015057945 “Hepatitis B viral assembly effector”.

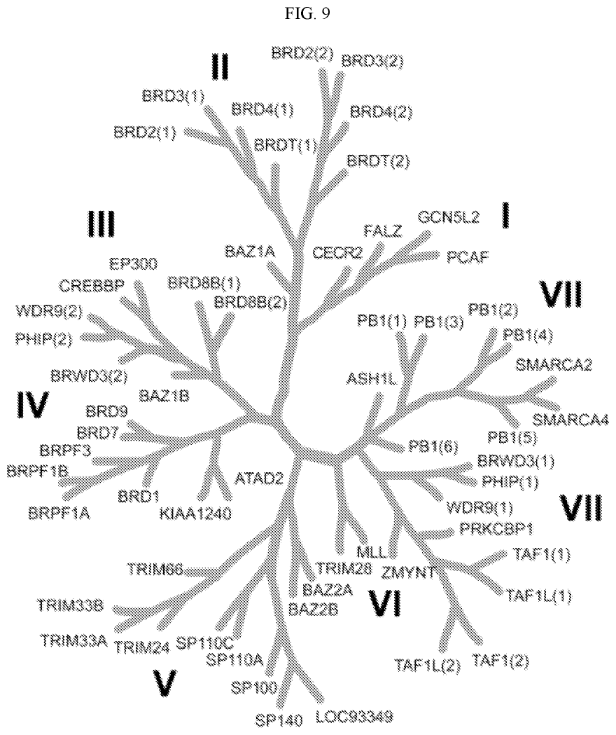

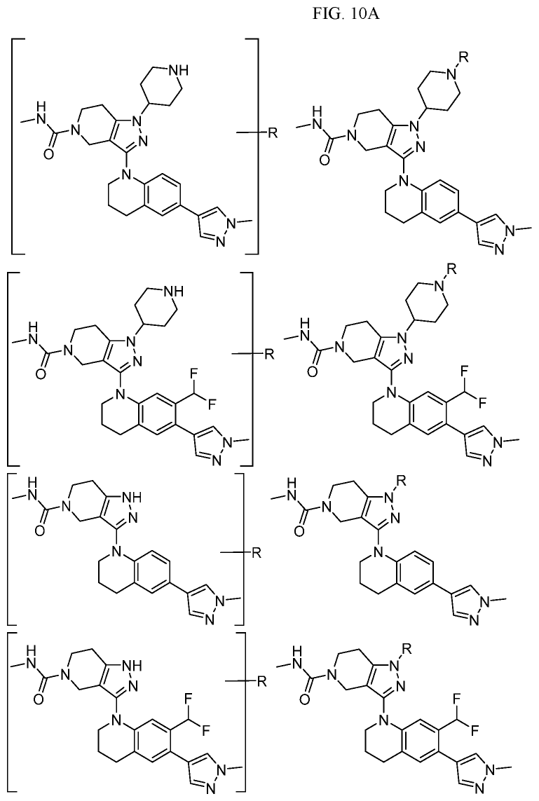

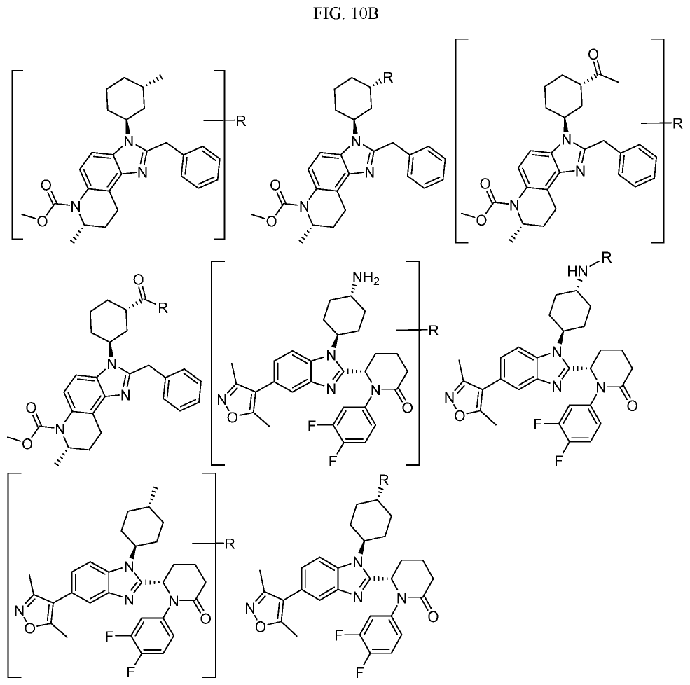

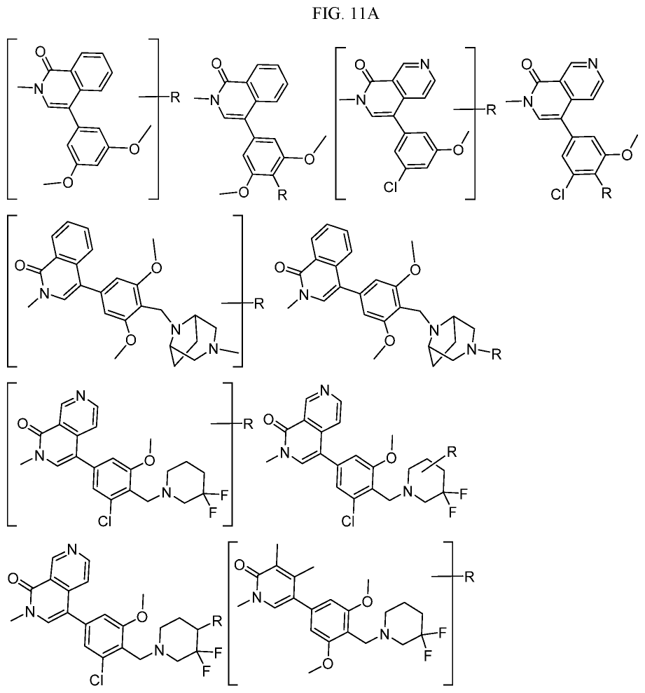

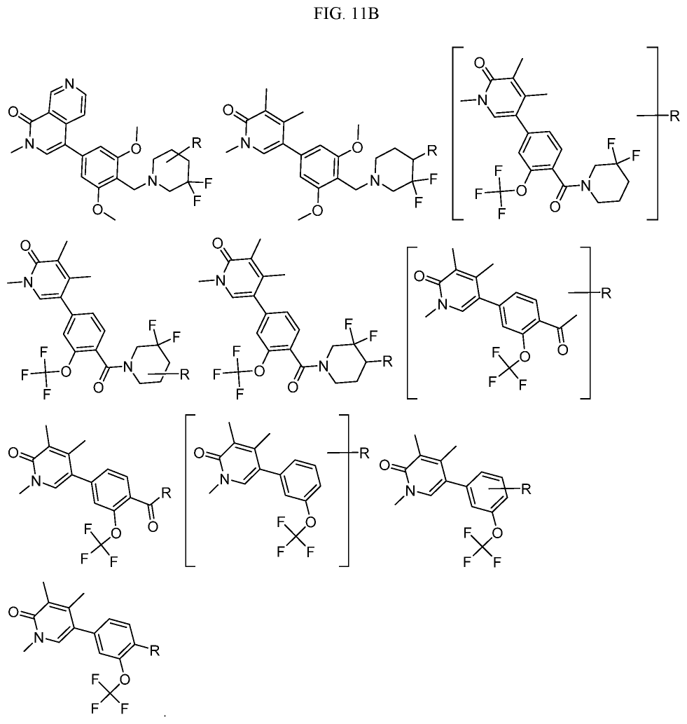

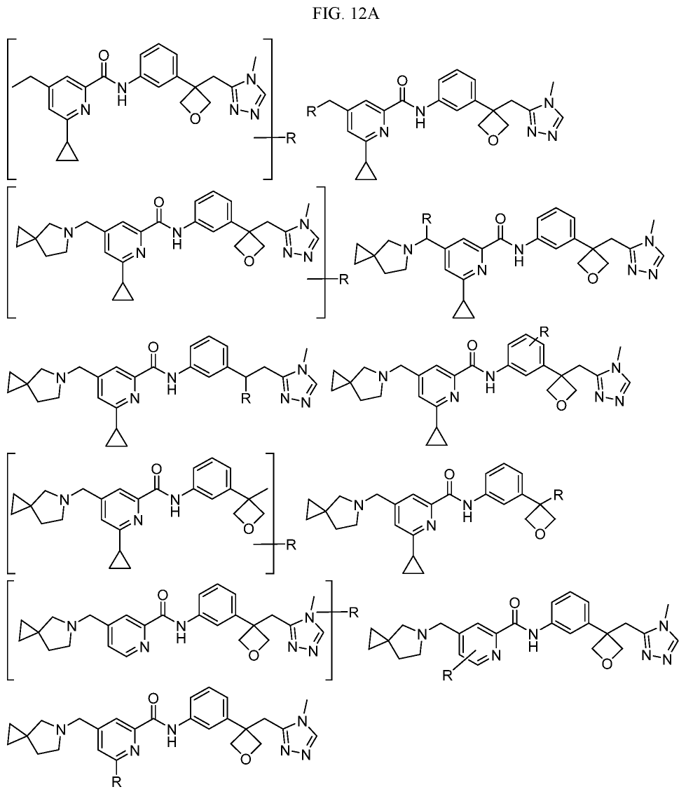

FIG. 9 is a dendrogram of the human bromodomain family of proteins organized into eight sub families, which are involved in epigenetic signaling and chromatin biology. Any of the proteins of the bromodomain family in FIG. 9 can be selected as a Target Protein according to the present invention.

FIG. 10A and FIG. 10B provide non-limiting examples of CBP and/or P300 Targeting Ligands, wherein R represents exemplary points at which the spacer is attached. For example additional examples of Targeting Ligands see “GNE-781, A Highly Advanced Potent and Selective Bromodomain Inhibitor of Cyclic Adenosine Monophosphate Response Element Binding Protein, Binding Protein (CBP)” J Med Chem 2017, 60(22), 9162; CCS-1477, WO2018073586; FT-7051, and WO2019055869.

FIGS. 11A and 11B provide non-limiting examples of BRD9 Targeting Ligands wherein R is the point at which the Linker is attached. For additional examples see: “Structure-Based Design of an in Vivo Active Selective BRD9Inhibitor” J Med Chem 2016, 59(10), 4462; WO2016139361.

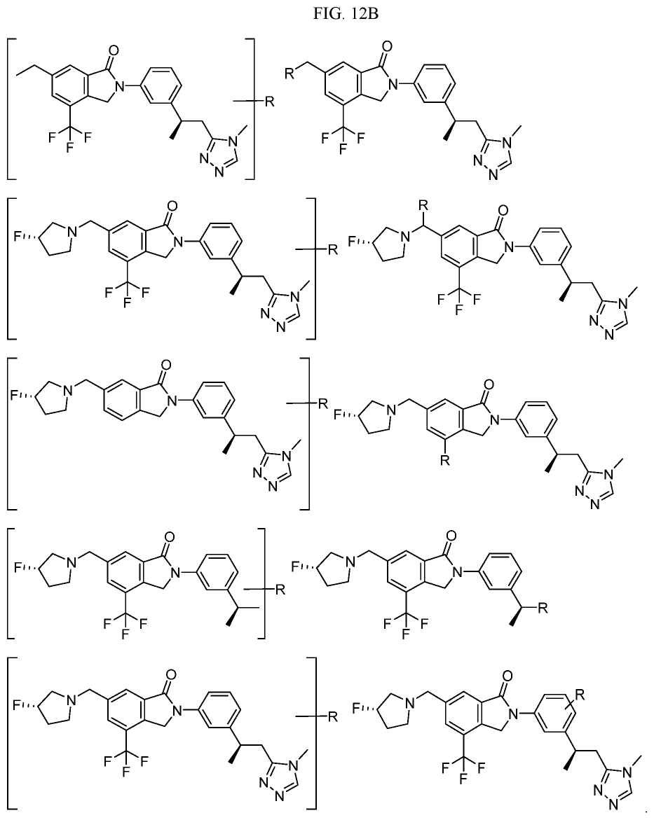

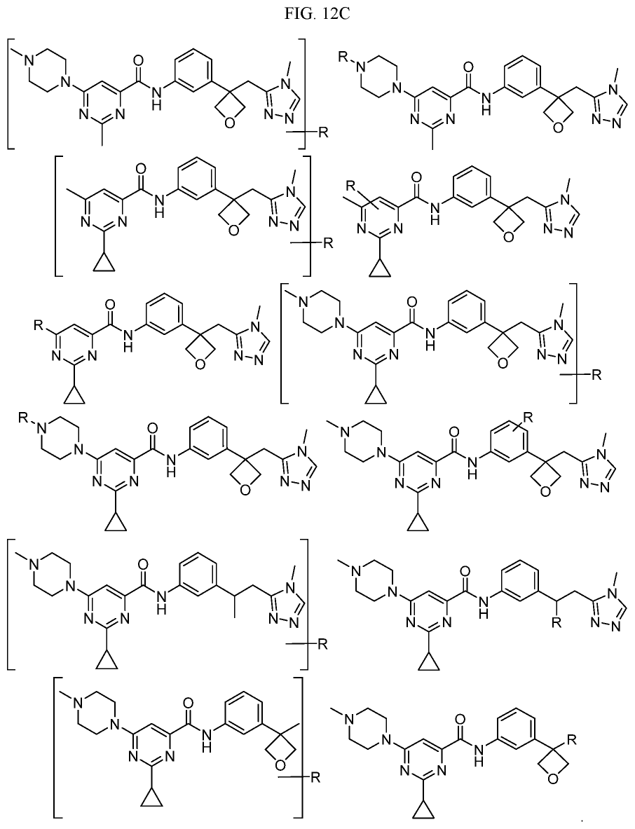

FIG. 12A-12C provide non-limiting examples of CBL-B Targeting Ligands, wherein R represents exemplary points at which the spacer is attached. For additional examples, see WO201914800).

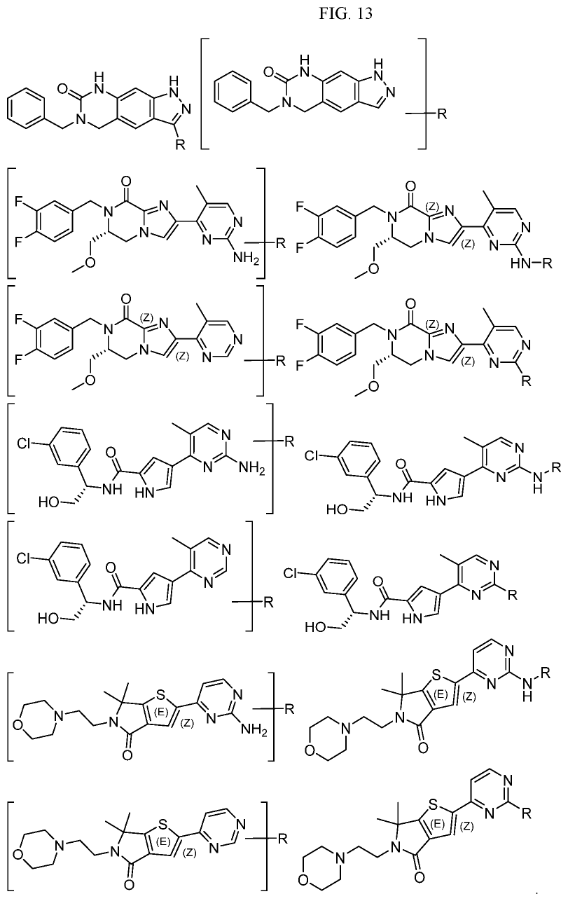

FIG. 13 provides non-limiting examples of ERK Targeting Ligands wherein R is the point at which the Linker is attached. For additional examples see: “Structure-Guided Design ofPotent and Selective Pyrimidylpyrrole Inhibitors of Extracellular Signal-Regulated Kinase (ERK) Using Conformational Control” J Med Chem 2009, 52(20), 6362; WO2015051341; “Discovery of a Potent and Selective Oral Inhibitor of ERK1 2 (AZD0364) That Is Efficacious in Both Monotherapy and Combination Therapy in Models of Nonsmall Cell Lung Cancer (NSCLC)” J Med Chem 2019, 62(24), 11004; “ERK Inhibitor LY3214996 Targets ERK Pathway-Driven Cancers: A Therapeutic Approach Toward Precision Medicine” Mol Cancer Ther 2020, 19, 325.

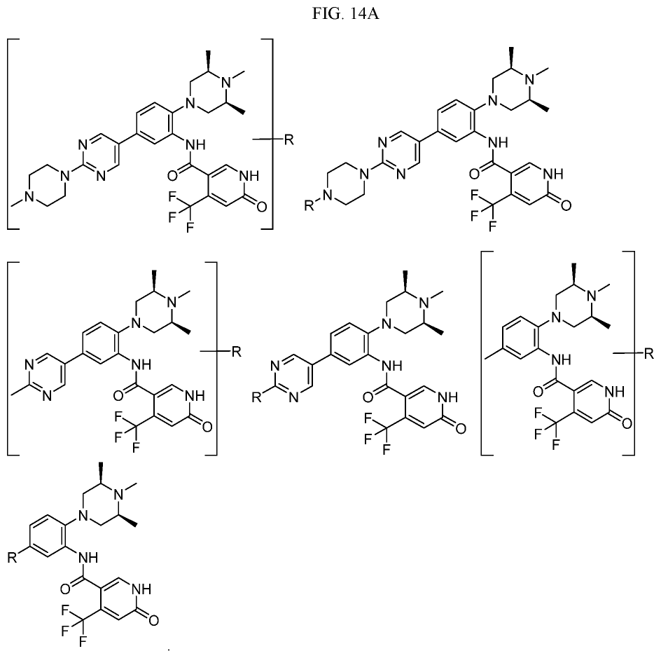

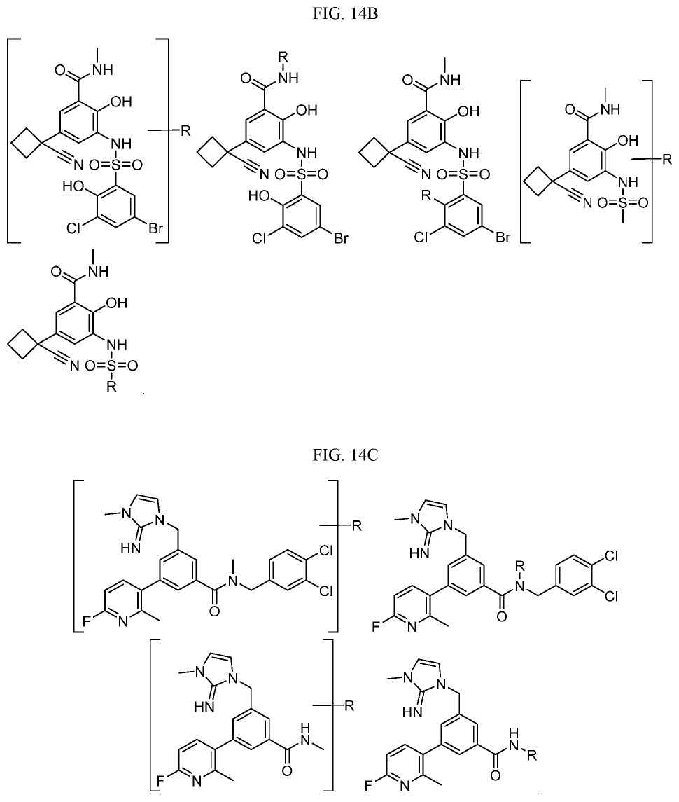

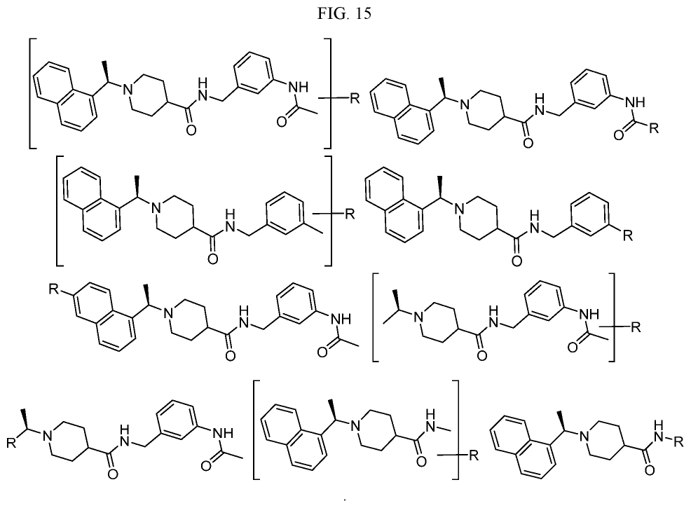

FIG. 14A-14C provide non-limiting examples of WDR5 Targeting Ligands, wherein R represents exemplary points at which the spacer is attached. For additional examples see “Structure-Based Optimization of a Small Molecule Antagonist of the Interaction Between WD Repeat-Containing Protein 5 (WDR5) and Mixed-Lineage Leukemia 1 (MLL1)” J Med Chem 2016, 59(6), 2478; WO2017147700; “Displacement of WDR5 from Chromatin by a WIN Site Inhibitor with Picomolar Affinity” Cell Rep 2019, 26(11), 2916; “Discovery and Optimization of Salicylic Acid-Derived Sulfonamide Inhibitors of the WD Repeat-Containing Protein 5-MYC Protein-Protein Interaction” J Med Chem 2019, 62(24), 11232).

FIG. 15 provides non-limiting examples of NSP3 Targeting Ligands, wherein R represents exemplary points at which the spacer is attached. For additional examples see: “Severe Acute Respiratory Syndrome Coronavirus Papain-like Novel Protease Inhibitors: Design, Synthesis, Protein-Ligand X-ray Structure and Biological Evaluation”, J Med Chem 2010, 53, 4968; “X-ray Structural and Biological Evaluation of a Series of Potent and Highly Selective Inhibitors of Human Coronavirus Papain-like Proteases”, J Med Chem 2014, 57, 2393).

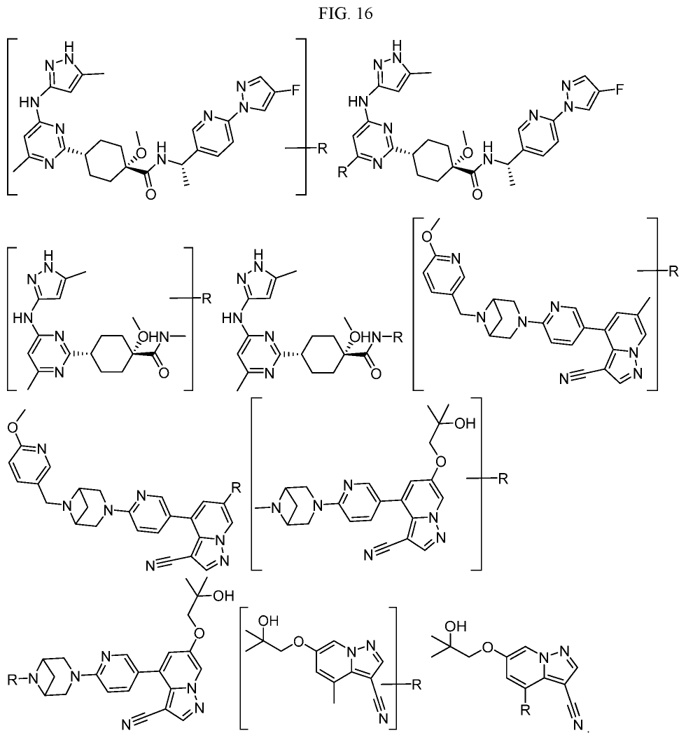

FIG. 16 provides non-limiting examples of RET Targeting Ligands, wherein R represents exemplary points at which the spacer is attached. For additional examples see: Pralsetinib “Precision Targeted Therapy with BLU-667 for RET-Driven Cancers” Cancer Discovery, 2018, 8(7), 836; Selpercatinib, WO2018071447; “A Pyrazolo[3,4-d]pyrimidin-4-amine Derivative Containing an Isoxazole Moiety Is a Selective and Potent Inhibitor of RET Gatekeeper Mutants” J Med Chem, 2016, 59, 358).

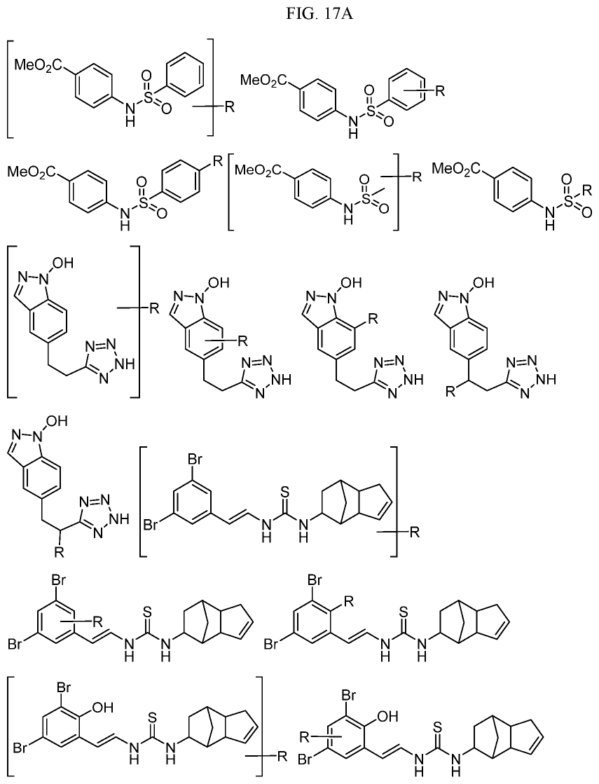

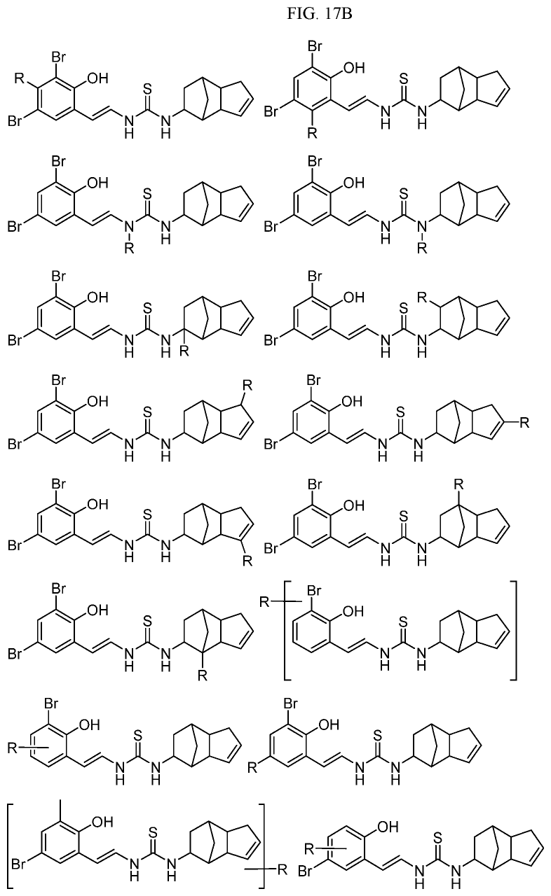

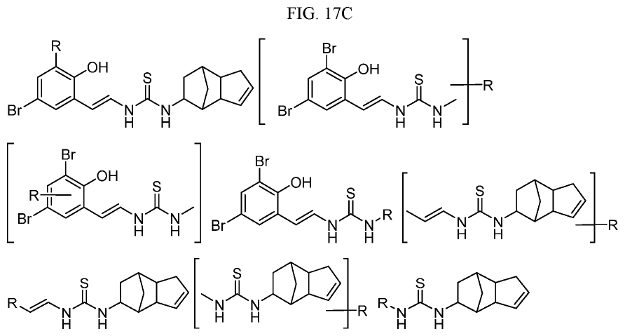

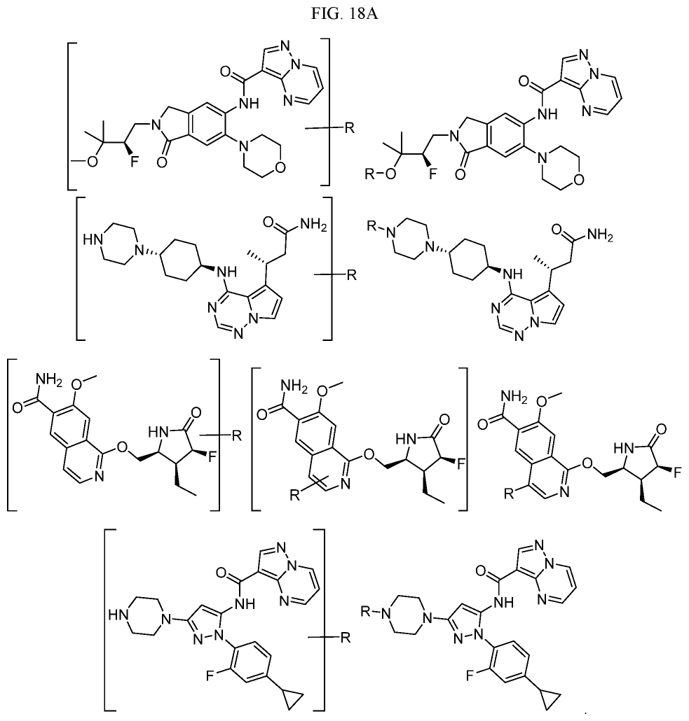

FIG. 17A-17C provide non-limiting examples of CTNNB1 Targeting Ligands wherein R is the point at which the Linker is attached. For additional examples see: “Direct Targeting of b-Catenin by a Small Molecule Stimulates Proteasomal Degradation and Suppresses Oncogenic Wnt/b-Catenin Signaling” Cell Rep 2016, 16(1), 28 “Rational Design of Small-Molecule Inhibitors for β-Catenin/T-Cell Factor Protein-Protein Interactions by Bioisostere Replacement” ACS Chem Biol 2013, 8, 524, and “Allosteric inhibitor of β-catenin selectively targets oncogenic Wnt signaling in colon cancer” Sci Rep 2020, 10, 8096.

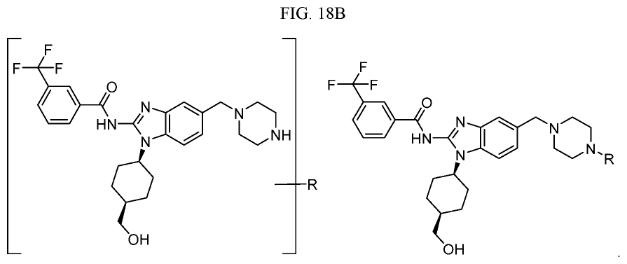

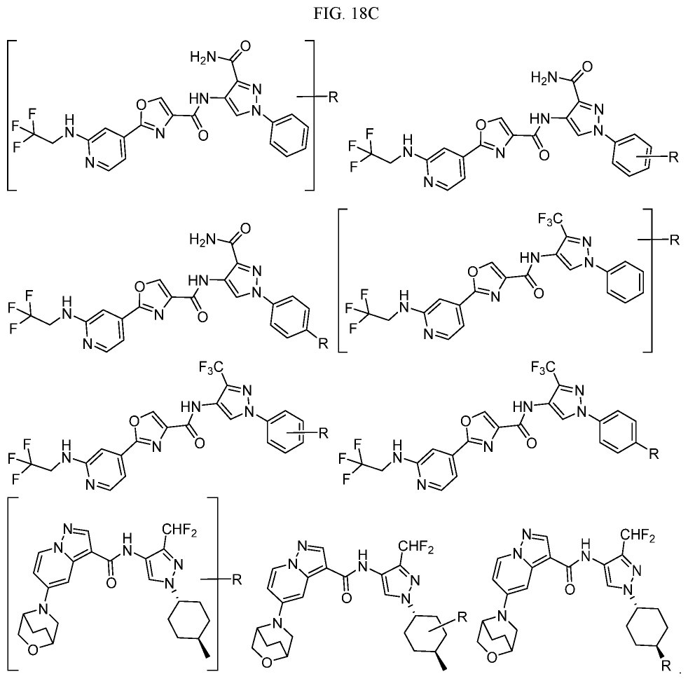

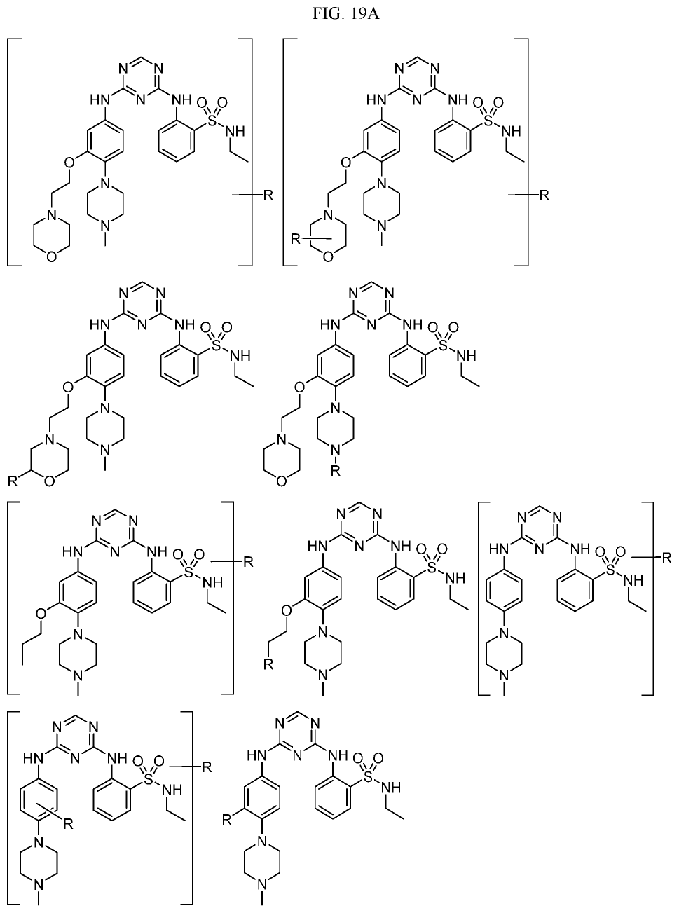

FIG. 18A-18C provide non-limiting examples of IRAK4 Targeting Ligands, wherein R represents exemplary points at which the spacer is attached. For additional examples and related ligands see crystal structures PDB 6UYA, 4YP8, 5UIU, and 6F3I in the respective references (Rajapaksa N. S. et al. “Discovery of Potent Benzolactam IRAK4 Inhibitors with Robust in Vivo Activity.” ACS Med. Chem. Lett. 11: 327-333 (2020); McElroy W. T. et al. “Potent and Selective Amidopyrazole Inhibitors of IRAK4 That Are Efficacious in a Rodent Model of Inflammation.” ACS Med. Chem. Lett. 6: 677-682 (2015); Nunes J. et al. “Targeting IRAK4 for Degradation with PROTACs” ACS Med. Chem. Lett. 10: 1081-1085 (2019); 4); Degorce S. L. et al. “Optimization of permeability in a series of pyrrolotriazine inhibitors of IRAK4”. Bioorg. Med. Chem. 26: 913-924 (2018); WO2019099926 and WO2019133531.

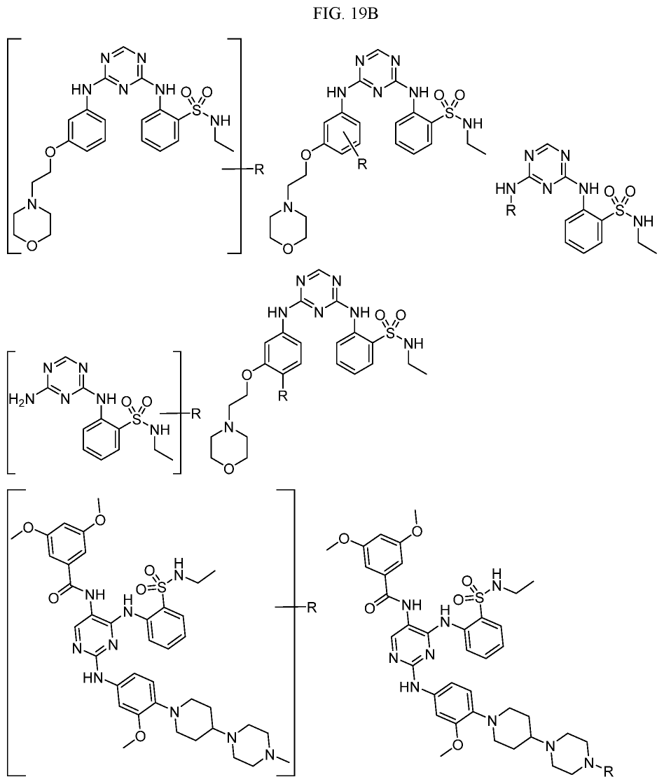

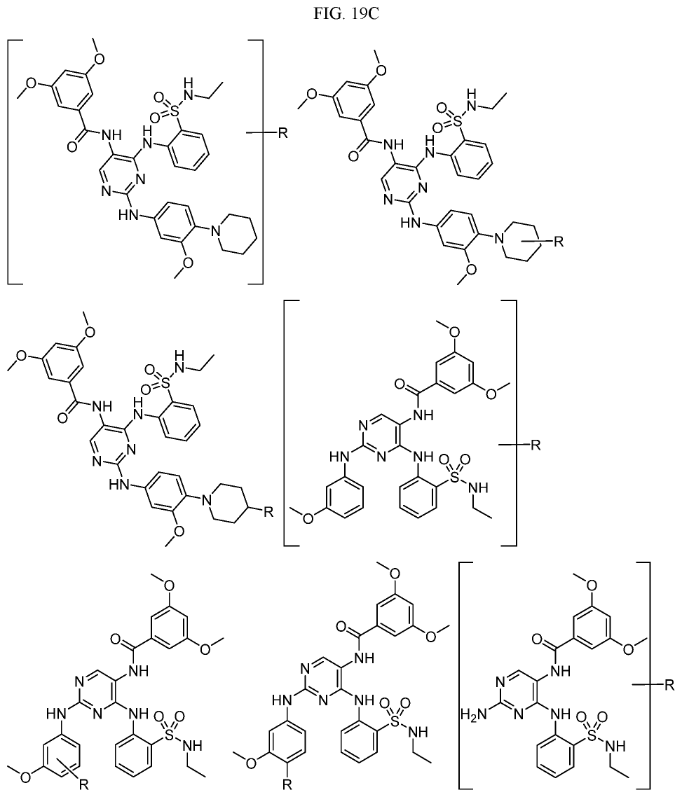

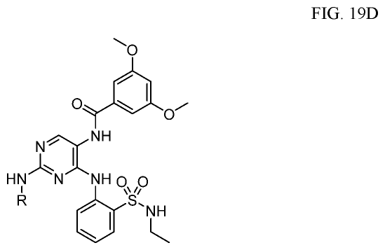

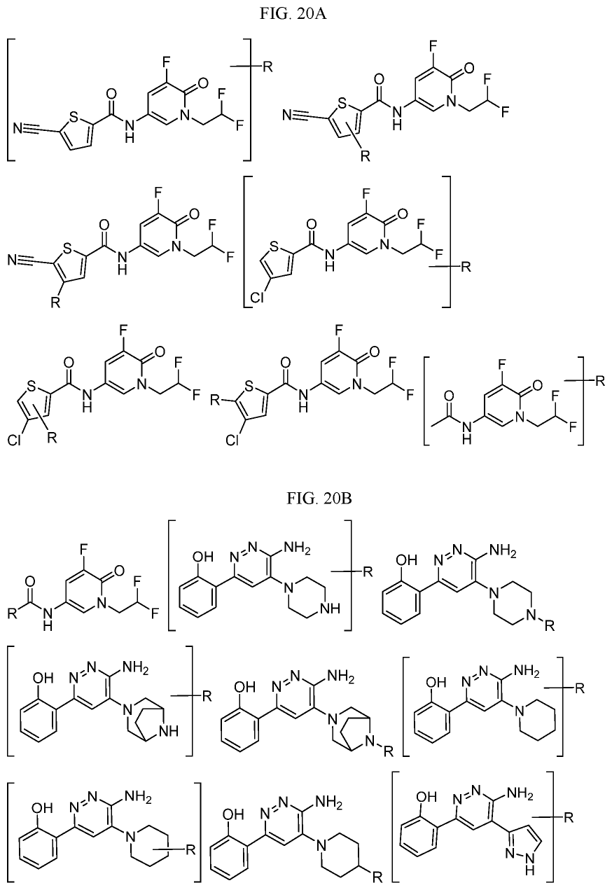

FIG. 19A-19D provide non-limiting examples of FGFR2 and FGFR3 Targeting Ligands wherein R is the point at which the Linker is attached. For additional examples see: “Structure-based drug design of 1,3,5-triazine and pyrimidine derivatives as novel FGFR3 inhibitors with high selectivity over VEGFR2” Bioorg Med Chem 2020, 28, 115453.

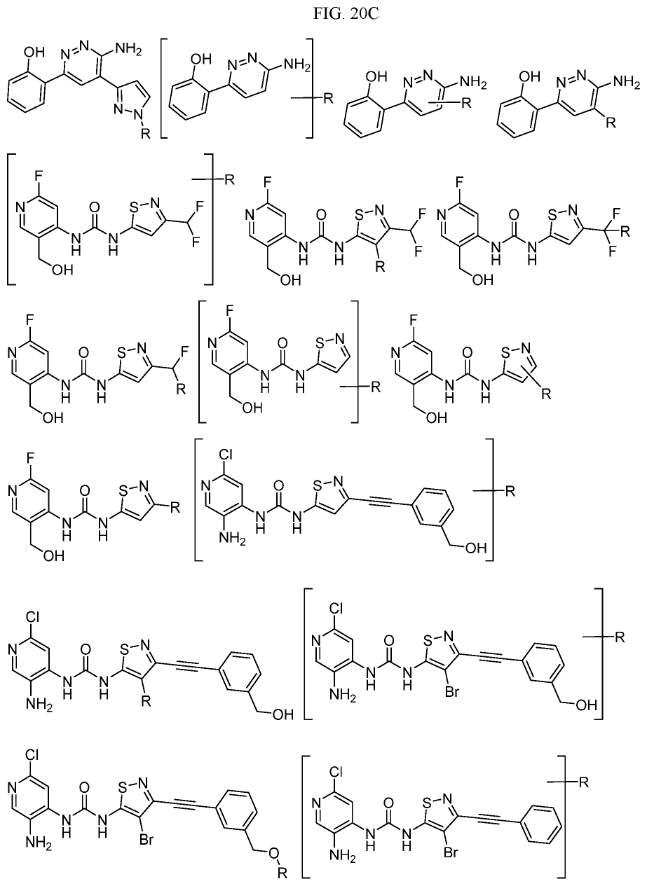

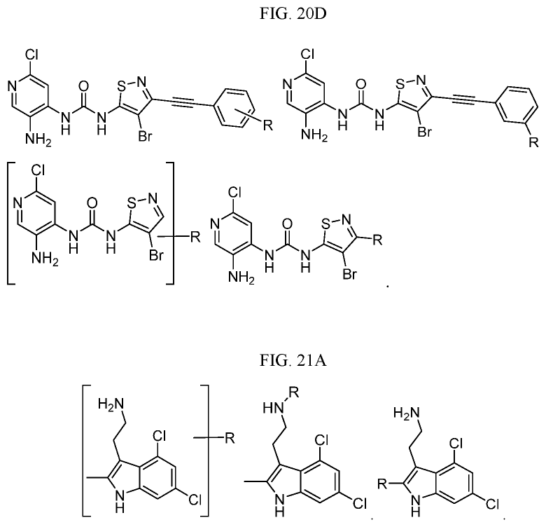

FIG. 20A-20D provide non-limiting examples of SMARCA2 Targeting Ligands wherein R is the point at which the Linker is attached. For additional examples see: WO2020023657, US20200038378, WO2020010227, WO2020078933, WO2019207538, WO2016138114, “Discovery of Orally Active Inhibitors of Brahma Homolog (BRM)/SMARCA2 ATPase Activity for the Treatment of Brahma Related Gene 1 (BRG1)/SMARCA4-Mutant Cancers” J Med Chem 2018, 61, 10155; 2) WO2020035779.

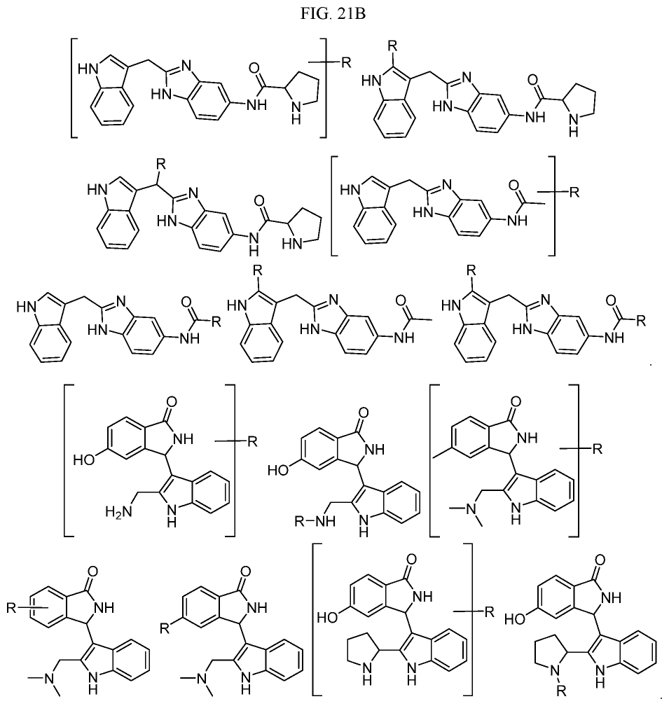

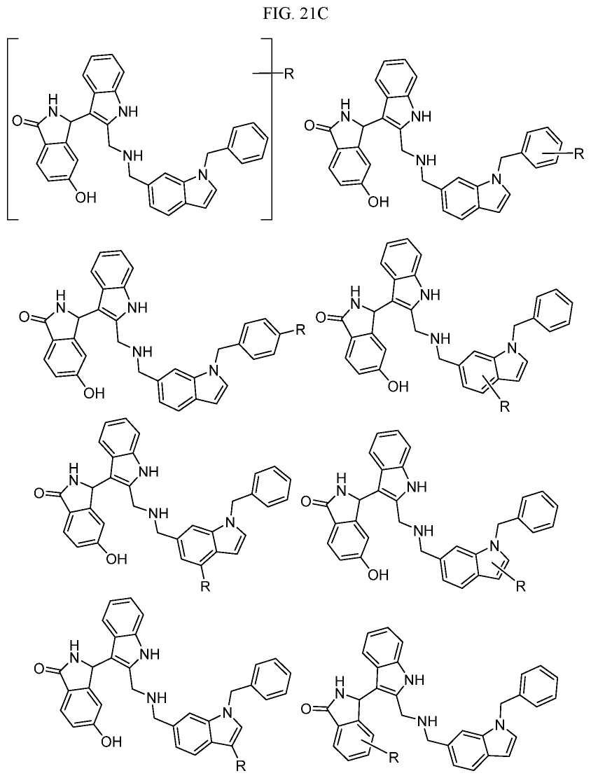

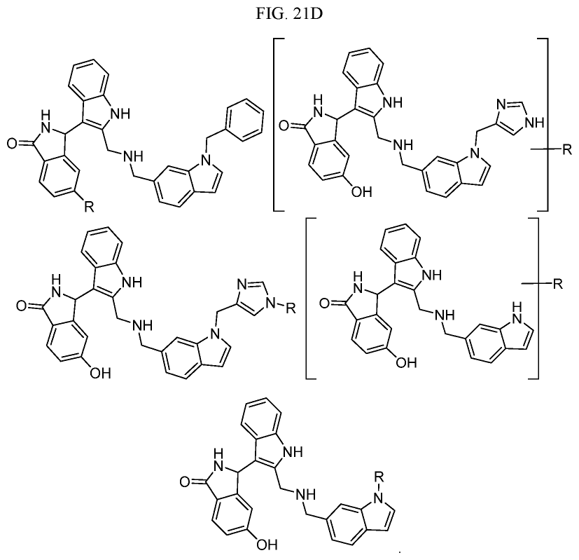

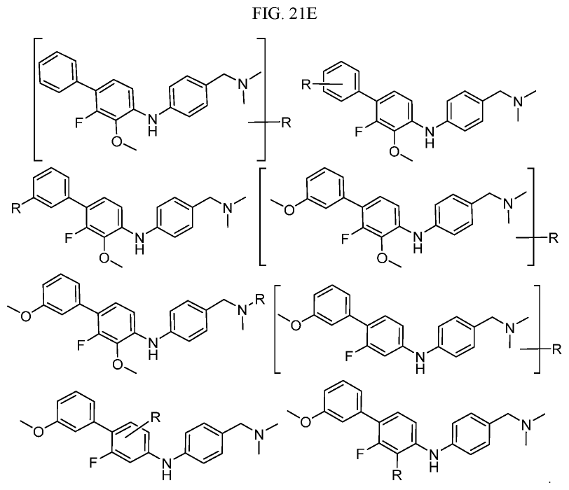

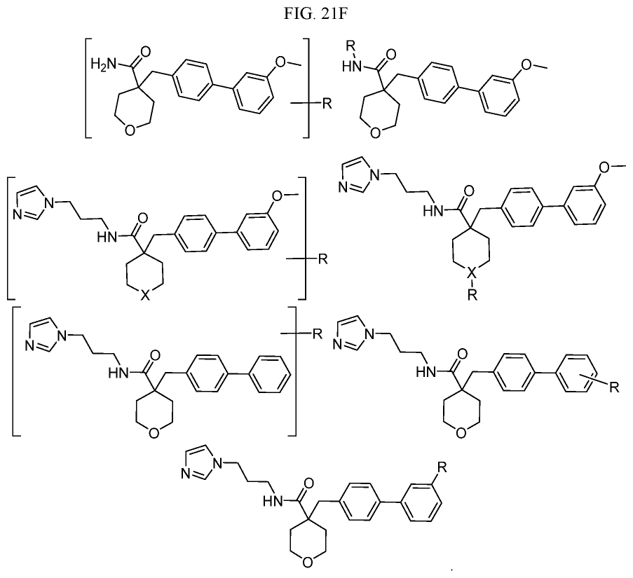

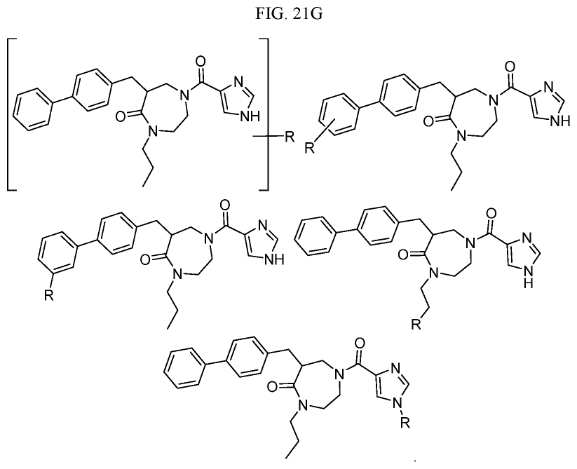

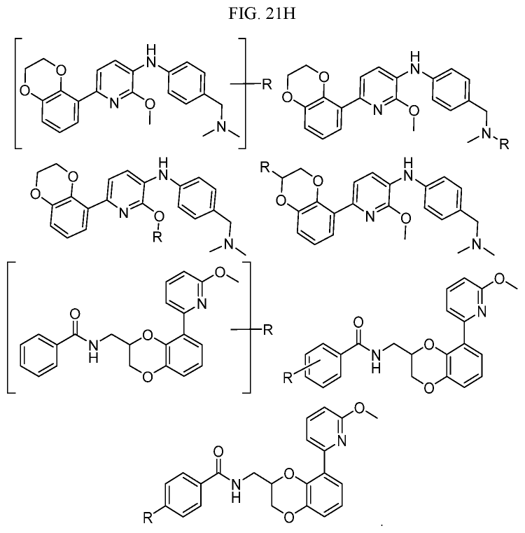

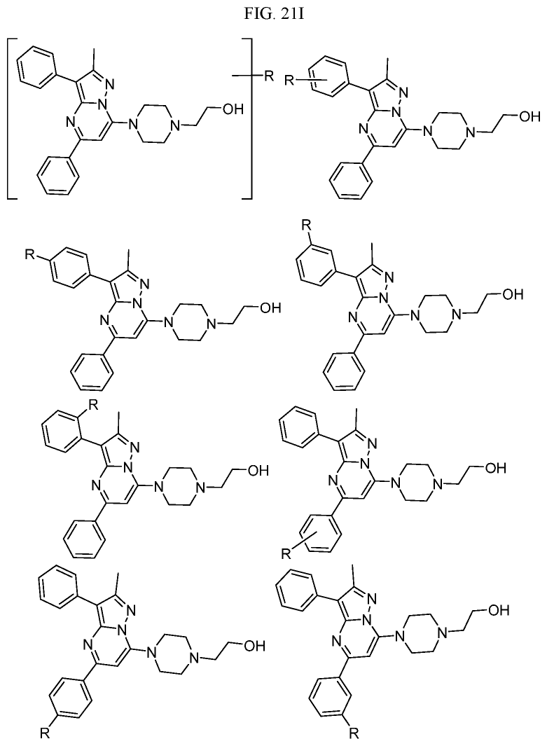

FIG. 21A-21J provide non-limiting examples of NRAS Targeting Ligands, wherein R represents exemplary points at which the spacer is attached. For additional examples, see “Small-molecule Ligands Bing to a Distinct Pocket in Ras and Inhibit SOS-Mediated Nucleotide Exchange Activity” PNAS 2012 109 (14) 5299-5304; the crystal structure PDB 4EPY. (“Discovery of Small Molecules that Bind to K-Ras and Inhibit Sos-Mediated Activation” Angew. Chem. Int. Ed 2012, 51, 6140-6143); the crystal structure PDB 6GQY, 6GQT, (“Structure-based development of new RAS-effector inhibitors from a combination of active and inactive RAS-binding compounds” 2019 PNAS 116 (7), 2545-2550); the crystal structure PDB 6FA4, 1HE8, (“Small molecule inhibitors of RAS-effector protein interactions derived using an intracellular antibody fragment” 2018 Nature Communications 9(1), 3169); and “Discovery of High-Affinity Noncovalent Allosteric KRAS Inhibitors That Disrupt Effector Binding” ACS Omega 2019, 4, 2921-2930.

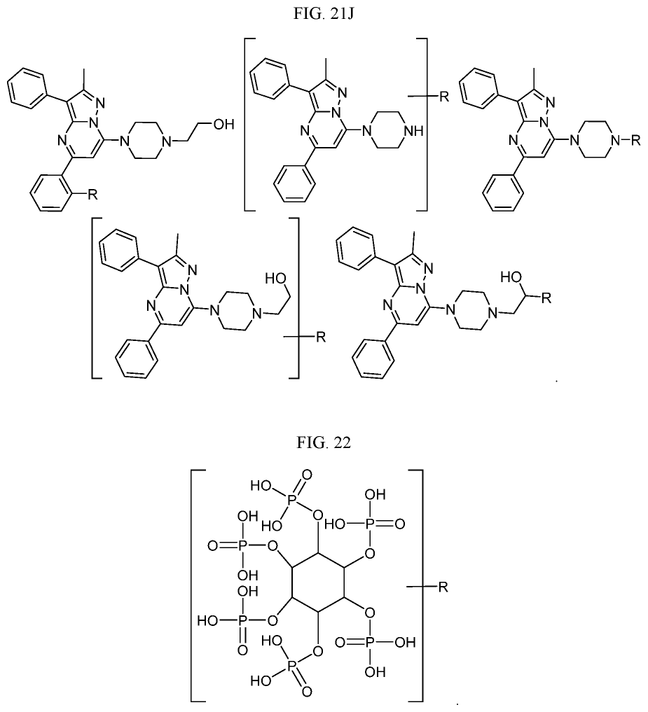

FIG. 22 provides a non-limiting example of an ADAR Targeting Ligand, wherein R represents exemplary points at which the spacer is attached. For additional examples, see the crystal structure PDB 6VFF, (Thuy-Boun, A. S., et al, Nucleic Acids Res, 2020, 48, 7958-7972); and the crystal structures PDB 51HP2, 51HP3, 5ED1, 5ED2 (Mathews, M. M, et al., Nat Struct Mol Biol., 2016, 23, 426-433).

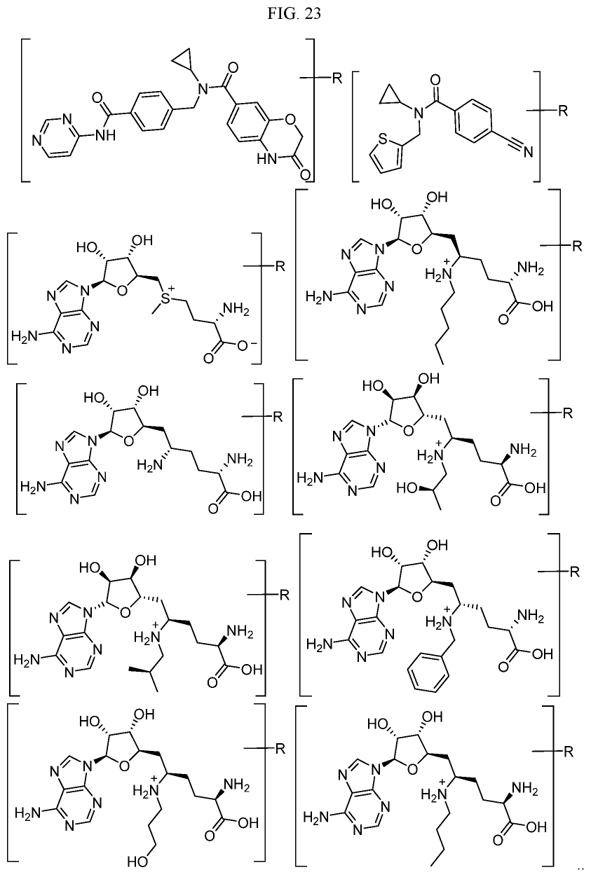

FIG. 23 provides non-limiting examples of NSD2 or WHSC1 Targeting Ligands, wherein R represents exemplary points at which the spacer is attached. For additional examples, see the crystal structure PDB 6XCG (Zhou, M. Q, et al., “Histone-lysine N-methyltransferase NSD2-PWWP1 with compound UNC6934”, to be published); the crystal structure PDB 6UE6 (Liu, Y et al., “PWWP1 domain of NSD2 in complex with MR837”, to be published); the crystal structure PDB 5LSS, 5LSU, 5LSX, 5LSY, 5LSZ, 5LT6,5LT7, 5LT8 (Tisi, D., et al, “Structure of the Epigenetic Oncogene MMSET and Inhibition by N-Alkyl Sinefungin Derivatives.”, ACS Chem Biol., 2016, 11: 3093-3105).

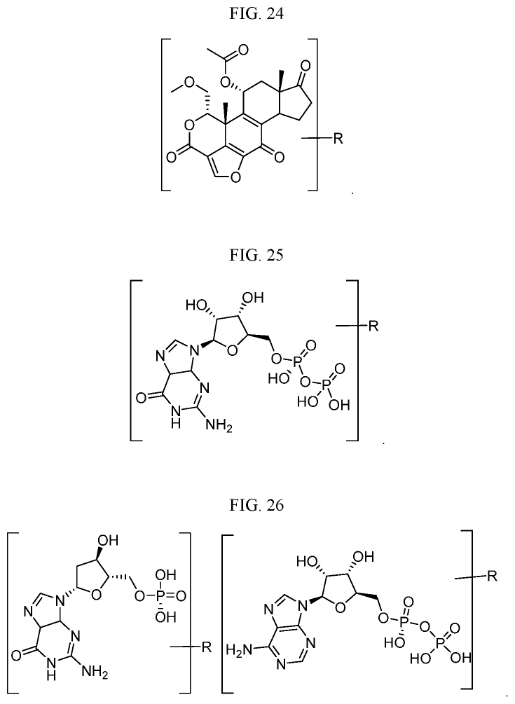

FIG. 24 provides non-limiting example of PI3KCA Targeting Ligands, wherein R represents exemplary points at which the spacer is attached. For additional examples, see the crystal structure PDB 3HHM, 3HIZ (Mandelker, D., et al., “A frequent kinase domain mutation that changes the interaction between PI3K{alpha} and the membrane.”, Proc Natl Acad Sci USA., 2009, 106: 16996-17001).

FIG. 25 provides a non-limiting example of a RIT1 Targeting Ligand, wherein R represents exemplary points at which the spacer is attached. For additional examples, see the crystal structure PDB 4KLZ (Shah, D. M., et al., “Inhibition of Small GTPases by Stabilization of the GDP Complex, a Novel Approach applied to Rit1, a Target for Rheumatoid Arthritis”, to be published).

FIG. 26 provides non-limiting examples of WRN Targeting Ligands, wherein R represents exemplary points at which the spacer is attached. For additional examples, see the crystal structure PDB 2FC0 (Perry, J. J., et al., “WRN exonuclease structure and molecular mechanism imply an editing role in DNA end processing.”, Nat Struct Mol Biol., 2006, 13: 414-422); and the crystal structure PDB 6YHR (Newman, J. A., et al., “Crystal structure of Werner syndrome helicase”, to be published).

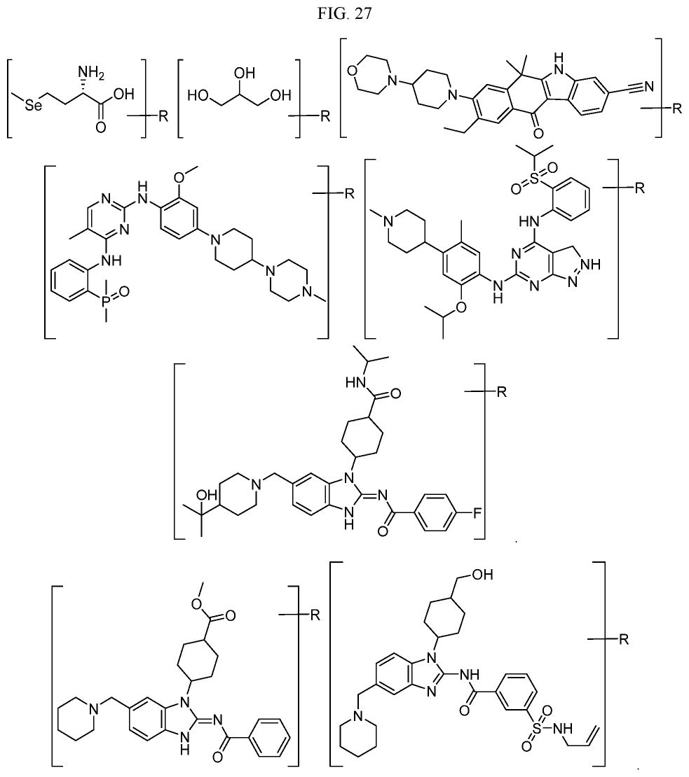

FIG. 27 provides non-limiting examples of ALK-fusion Targeting Ligands, for example EML4-ALK or NMP-ALK, wherein R represents exemplary points at which the spacer is attached. For additional examples, see the crystal structure PDB 4CGB, 4CGC (Richards, M. W., et al., “Microtubule Association of Eml Proteins and the Eml4-Alk Variant 3 Oncoprotein Require an N-Terminal Trimerization Domain”, Biochem J., 2015, 467: 529); the crystal structure PDB 3AOX (Sakamoto, H., et al., “CH5424802, a selective ALK inhibitor capable of blocking the resistant gatekeeper mutant”, Cancer Cell, 2011, 19: 679-690); the crystal structure PDB 6MX8 (Huang, W. S., et al., “Discovery of Brigatinib (AP26113), a Phosphine Oxide-Containing, Potent, Orally Active Inhibitor of Anaplastic Lymphoma Kinase”, J Med Chem., 2016, 59: 4948-4964); 4Z55 (Michellys, P. Y., et al., “Design and synthesis of novel selective anaplastic lymphoma kinase inhibitors.”, Bioorg Med Chem Lett., 2016, 26: 1090-1096); and the crystal structures PDB 4FOB, 4FOC, 4FOD (Lewis, R. T., et al, “The Discovery and Optimization of a Novel Class of Potent, Selective, and Orally Bioavailable Anaplastic Lymphoma Kinase (ALK) Inhibitors with Potential Utility for the Treatment of Cancer.”, J. Med. Chem., 2012, 55: 6523-6540).

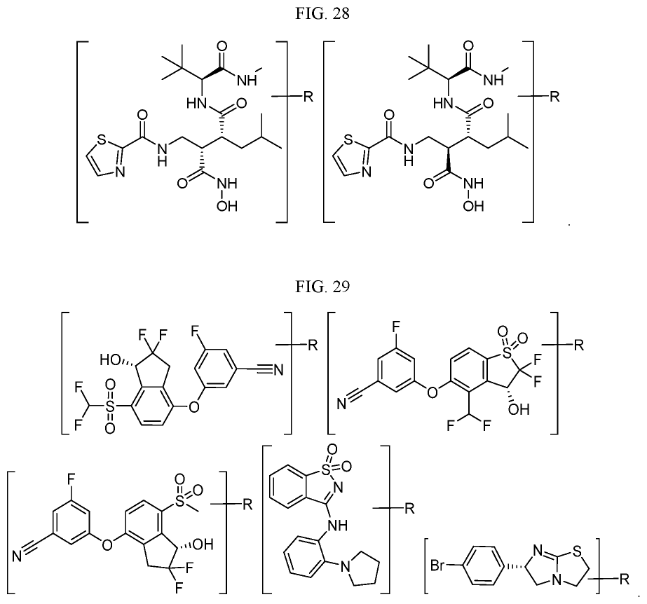

FIG. 28 provides non-limiting examples of BAP1 Targeting Ligands, wherein R represents exemplary points at which the spacer is attached. For additional examples, see the crystal structure PDB 2W12, 2W13, 2W14, 2W15 (Lingott, T. J. et al., “High-Resolution Crystal Structure of the Snake Venom Metalloproteinase Bap1 Complexed with a Peptidomimetic: Insight into Inhibitor Binding”, Biochemistry, 2009, 48: 6166).

FIG. 29 provides non-limiting examples of EPAS1 or HIF2a Targeting Ligands, wherein R represents exemplary points at which the spacer is attached. For additional examples, see the crystal structure PDB 5UFP (Cho, H., et al., “On-target efficacy of a HIF-2 alpha antagonist in preclinical kidney cancer models.”, Nature, 2016, 539: 107-111); the crystal structure PDB 6D09 Du, X, (“Crystal structure of PT1940 bound to HIF2a-B*:ARNT-B* complex”, to be published); the crystal structure PDB 5TBM (Wallace, E. M., et al., “A Small-Molecule Antagonist of HIF2 alpha Is Efficacious in Preclinical Models of Renal Cell Carcinoma.”, Cancer Res., 2016, 76: 5491-5500); and the crystal structure PDB 6E3S, 6E3T, 6E3U (Wu, D., et al., “Bidirectional modulation of HIF-2 activity through chemical ligands.”, Nat Chem Biol., 2019, 15: 367-376).

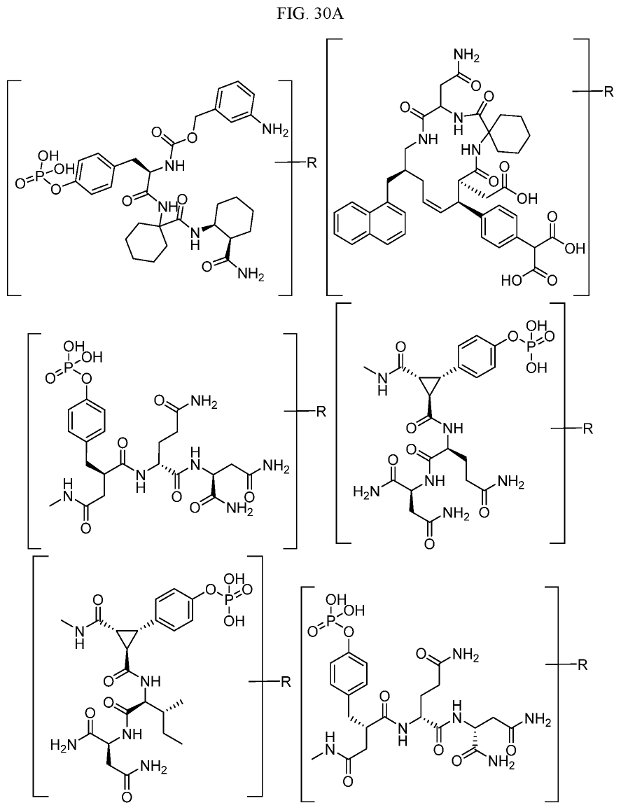

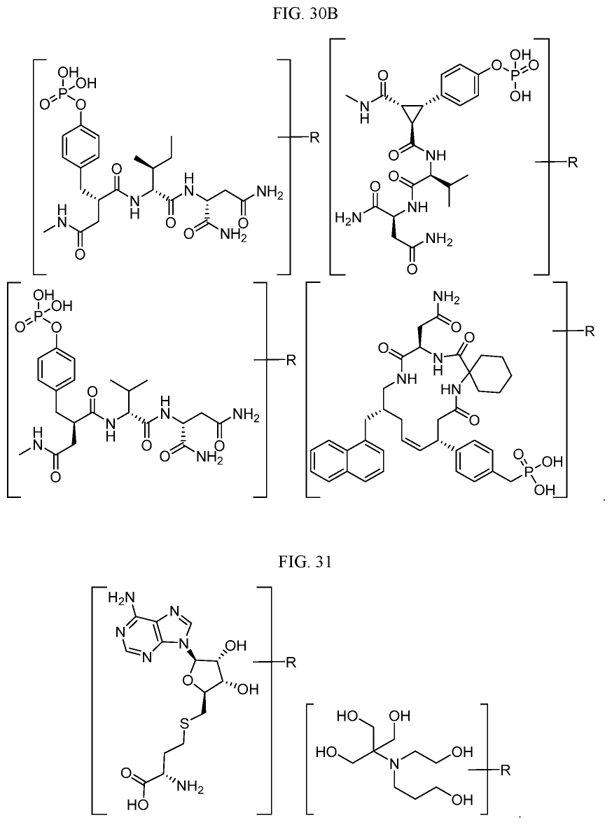

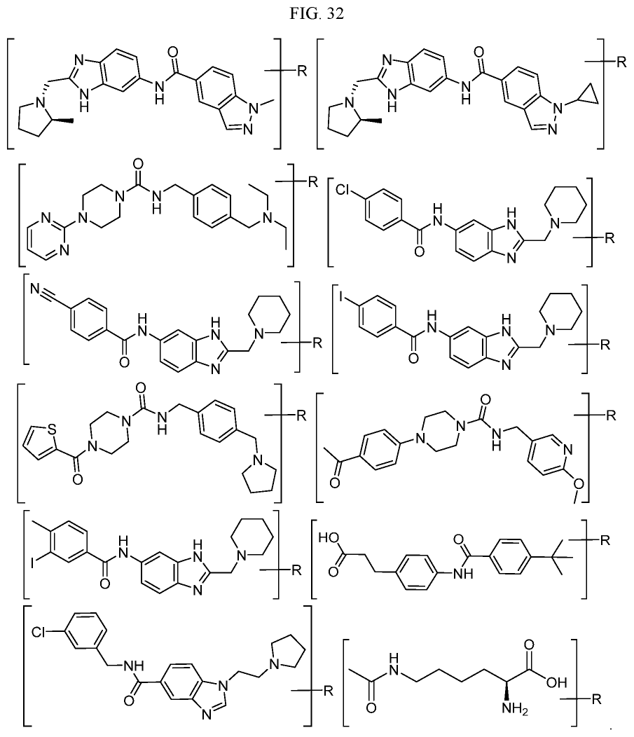

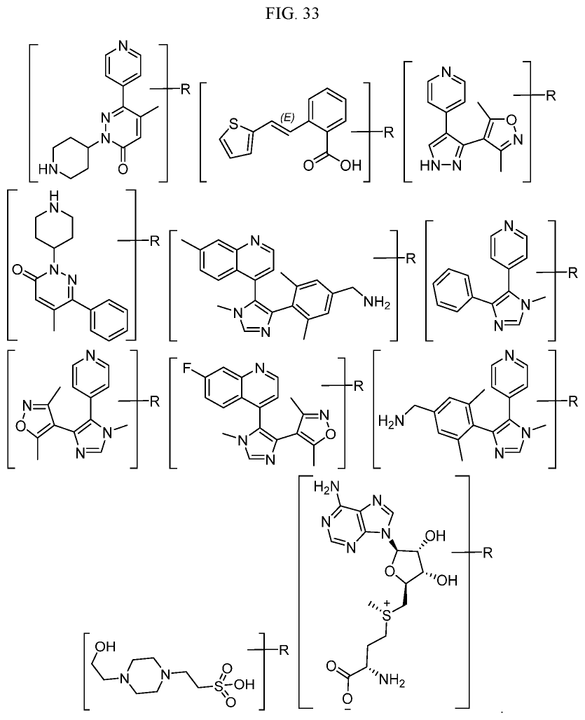

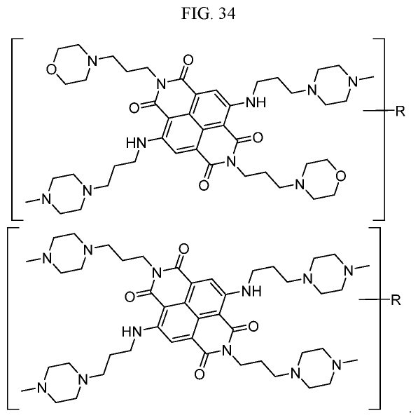

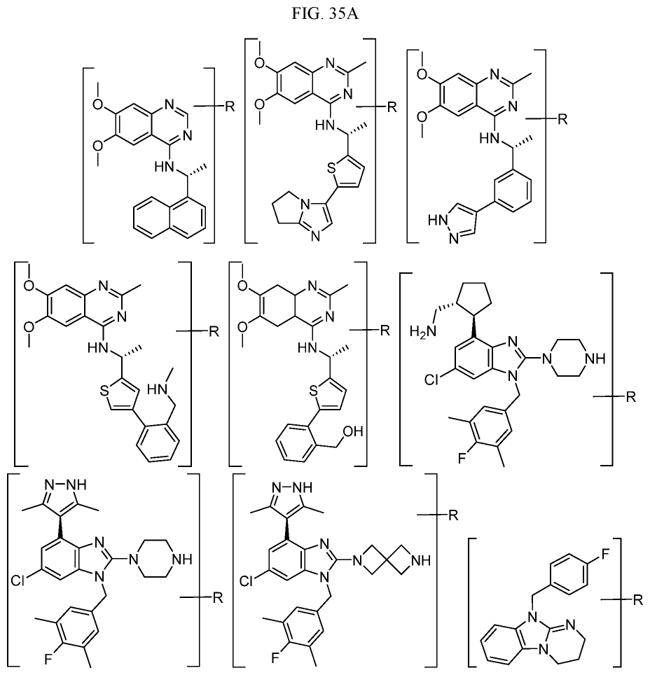

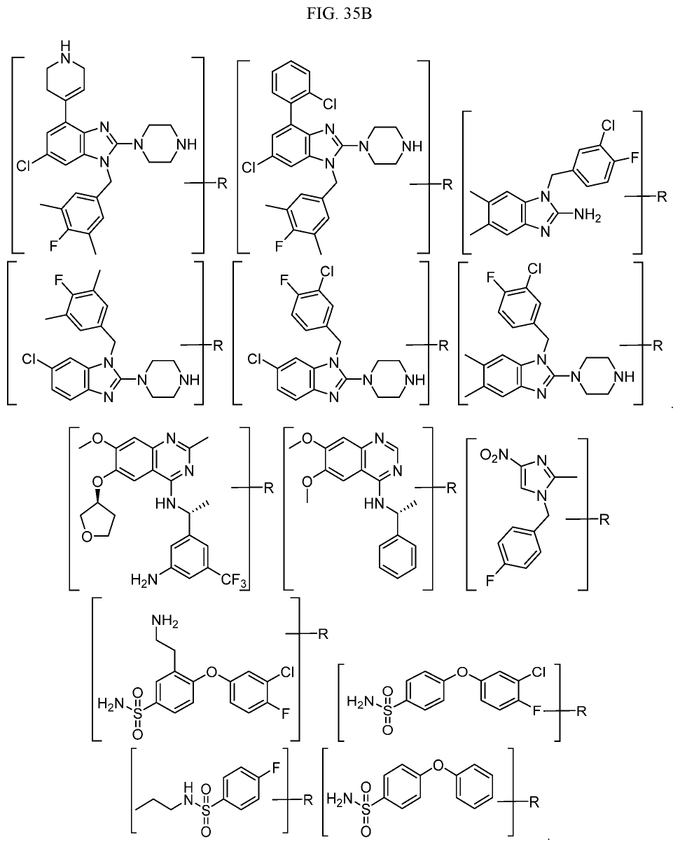

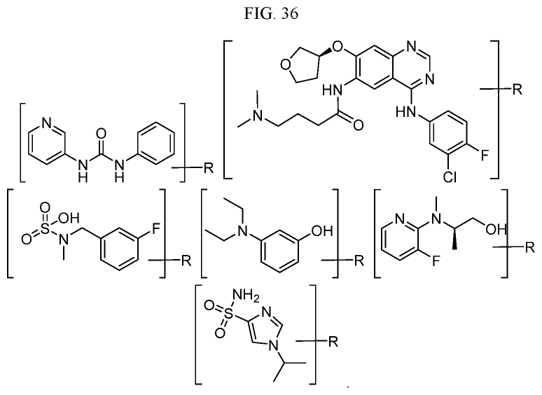

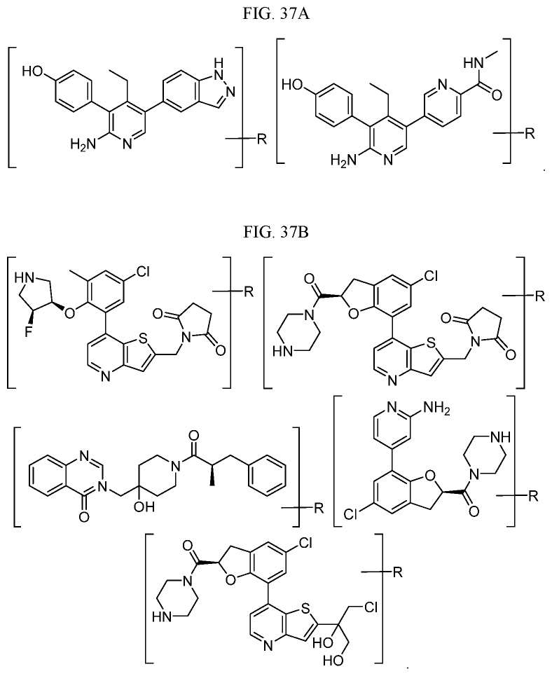

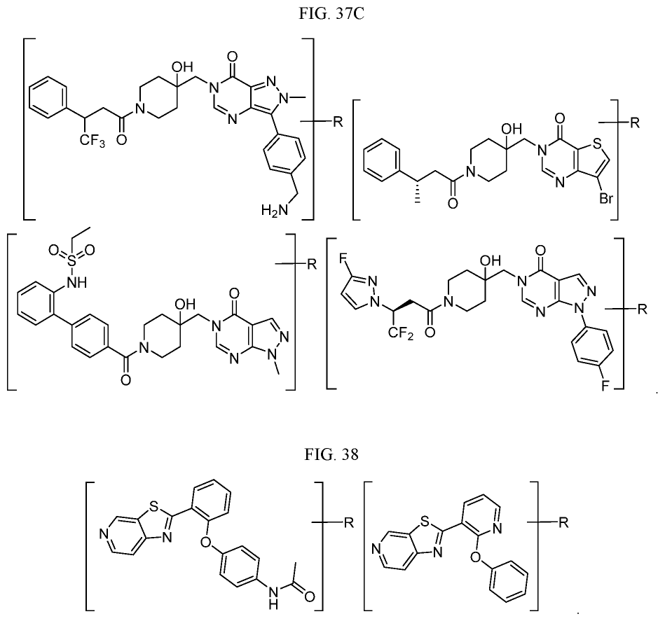

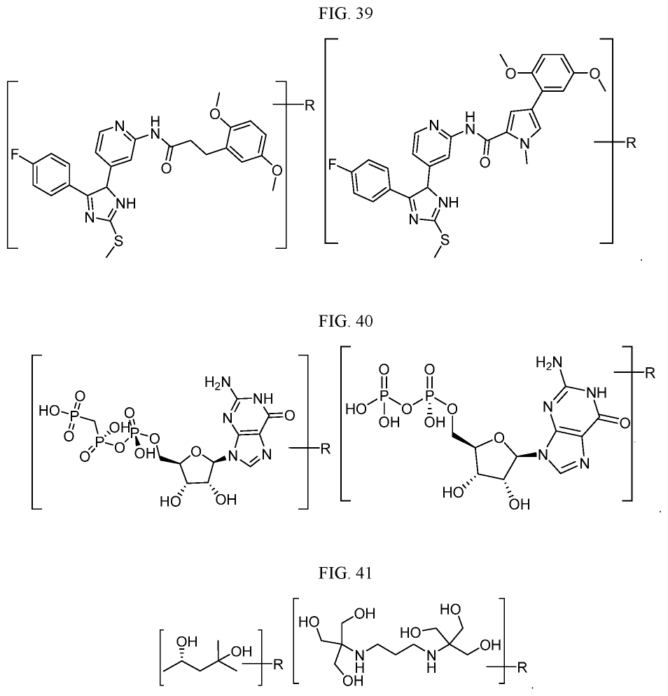

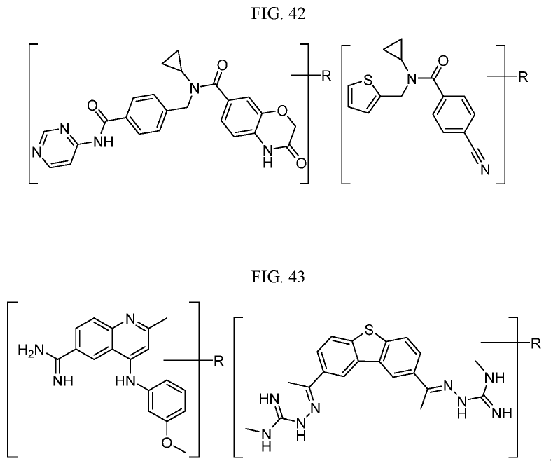

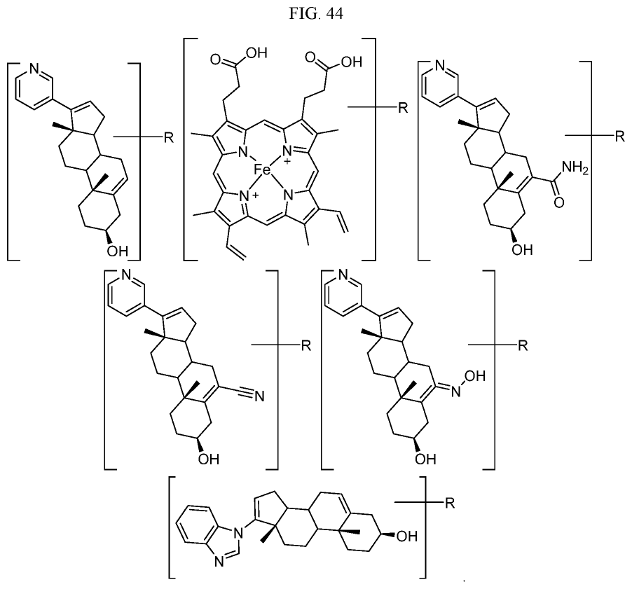

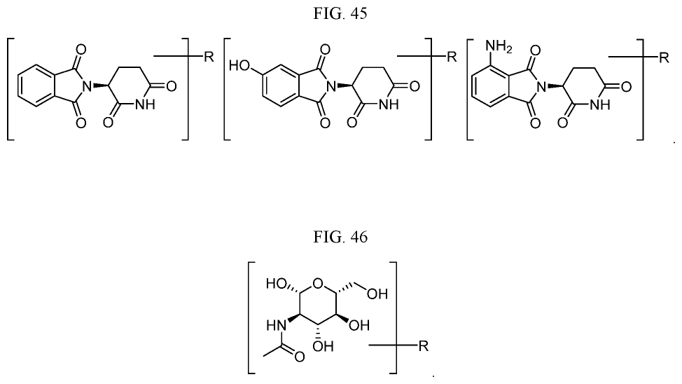

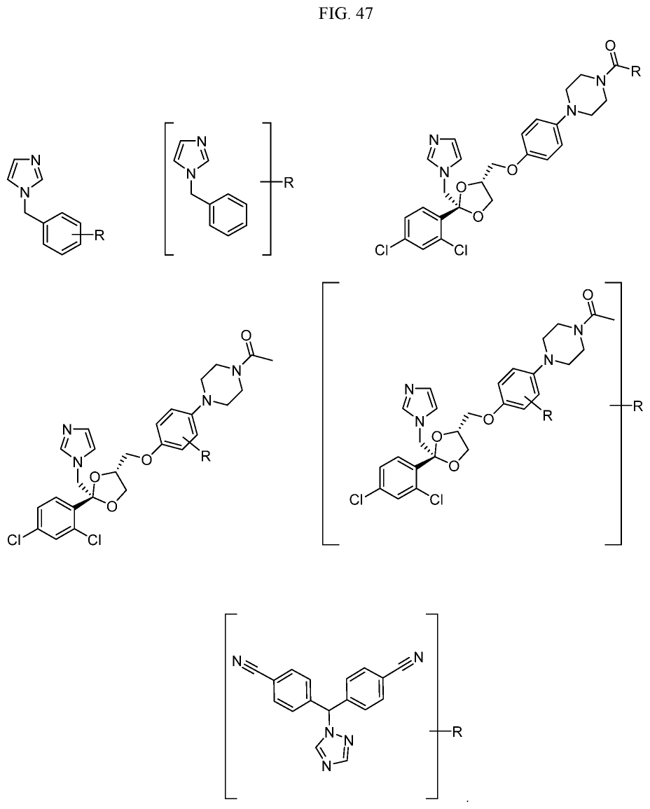

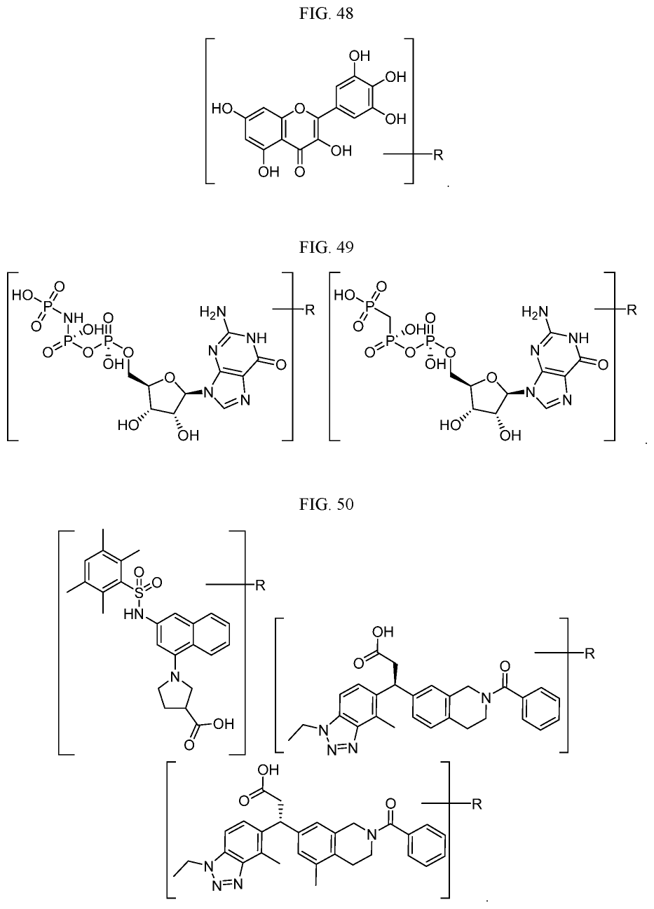

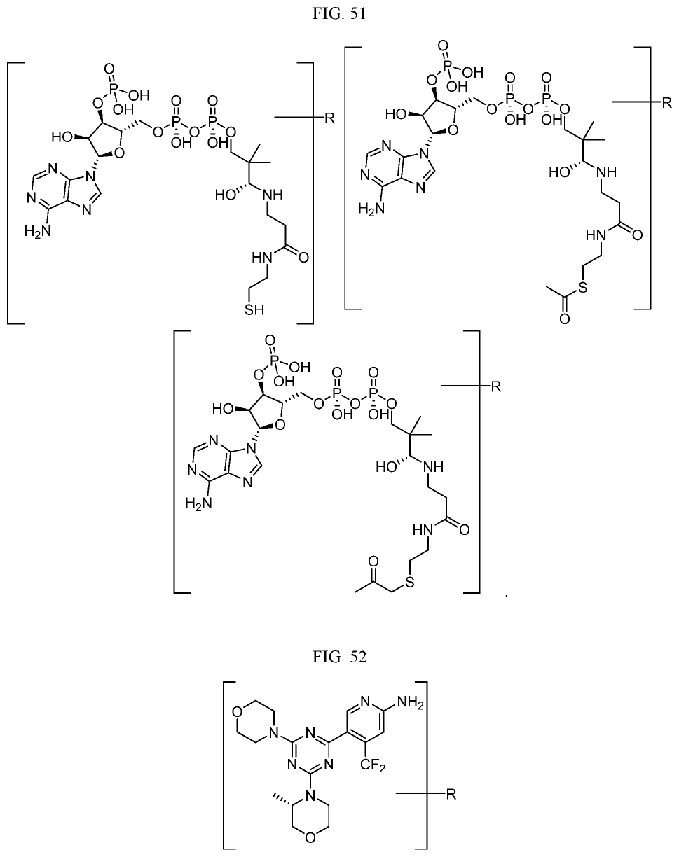

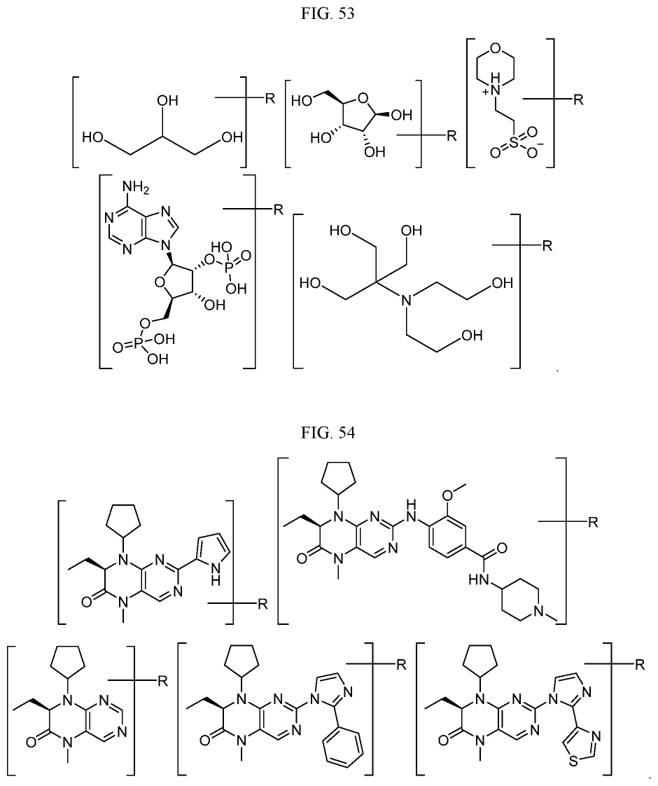

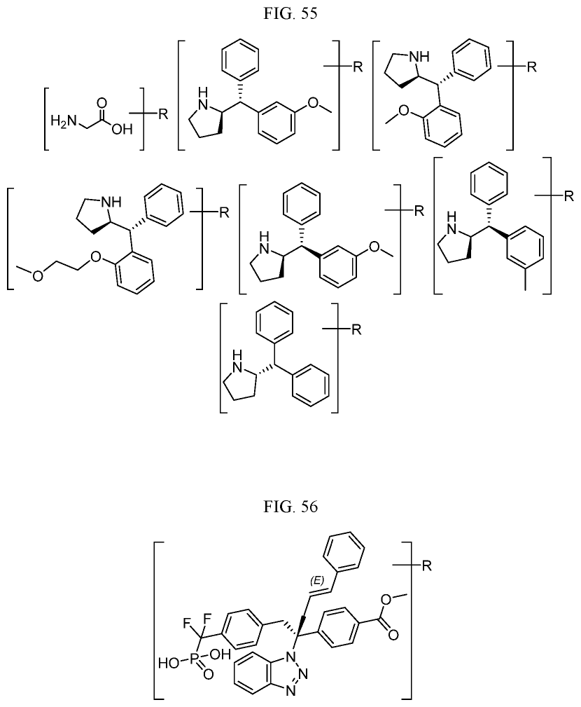

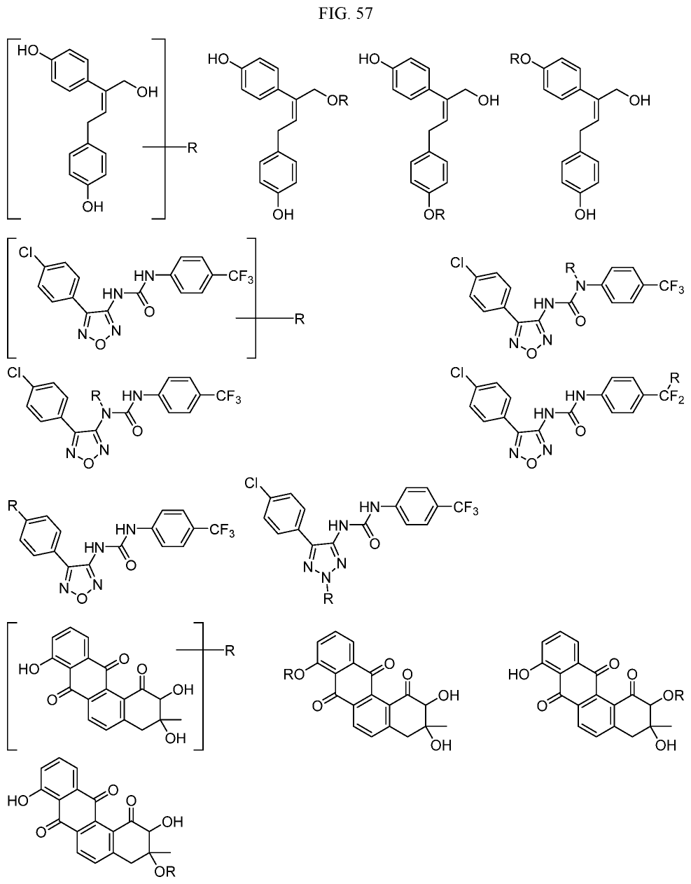

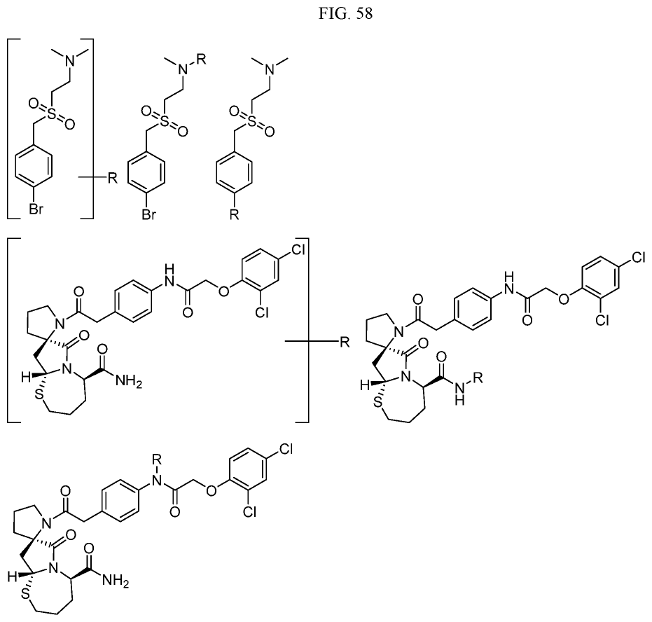

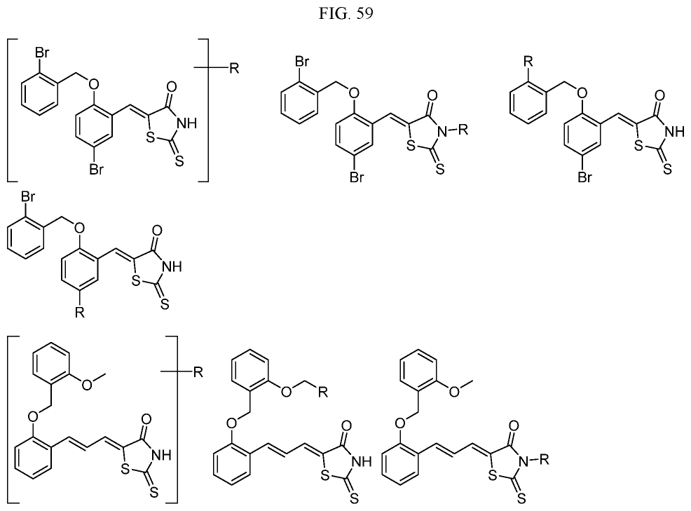

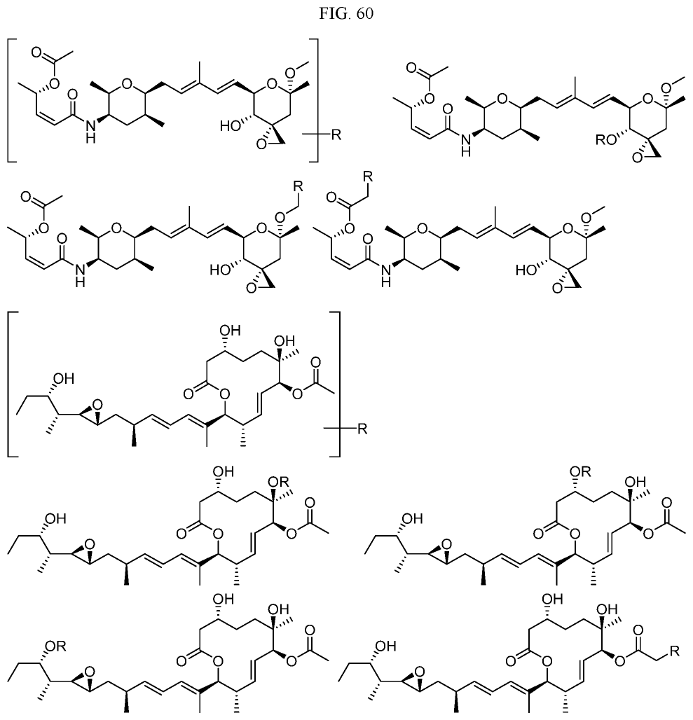

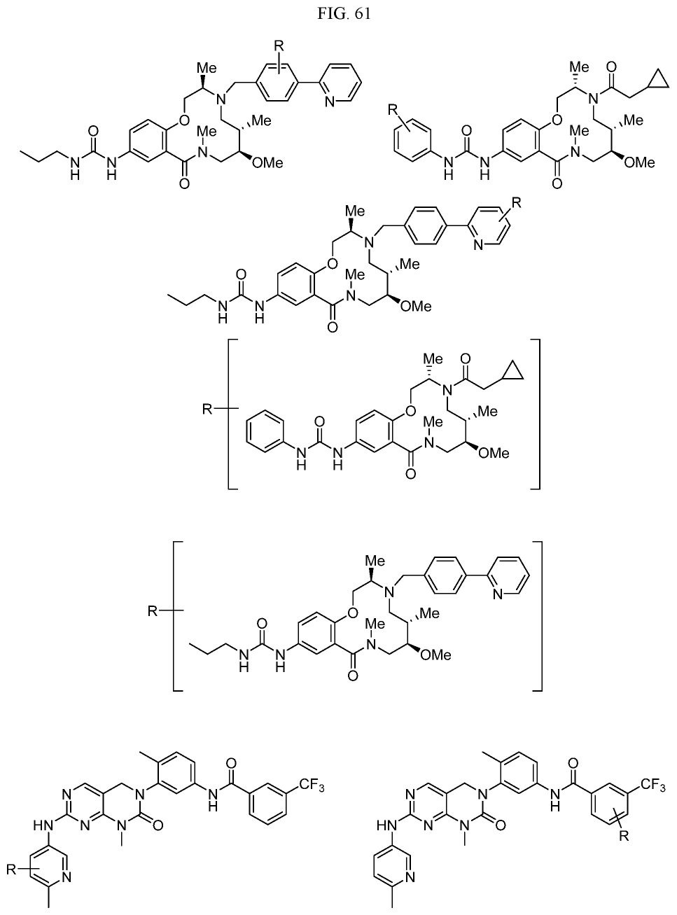

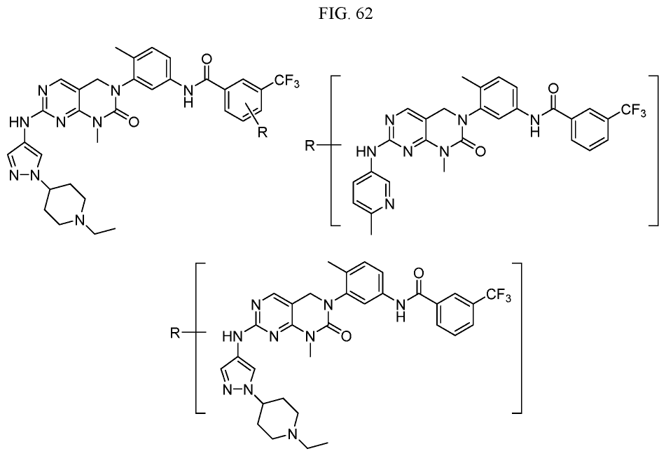

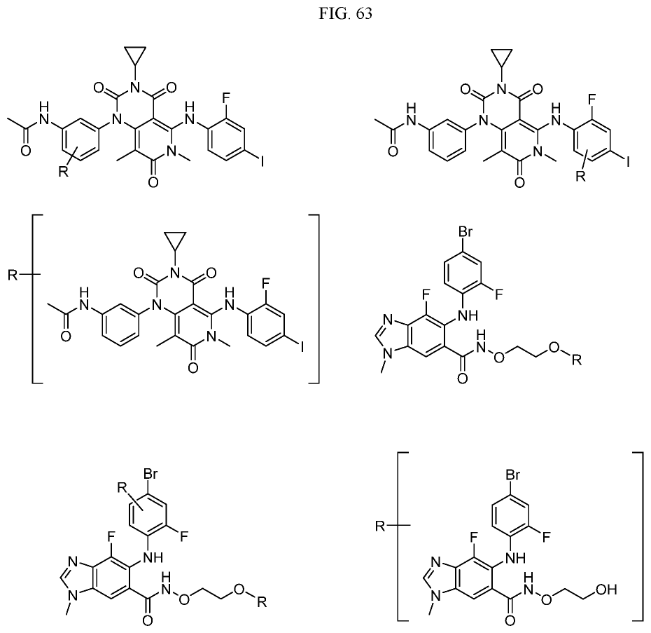

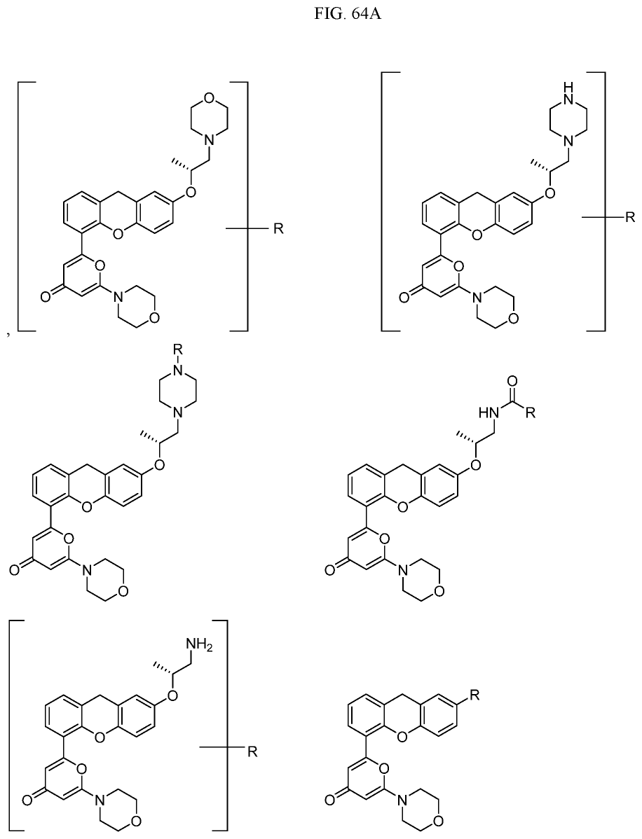

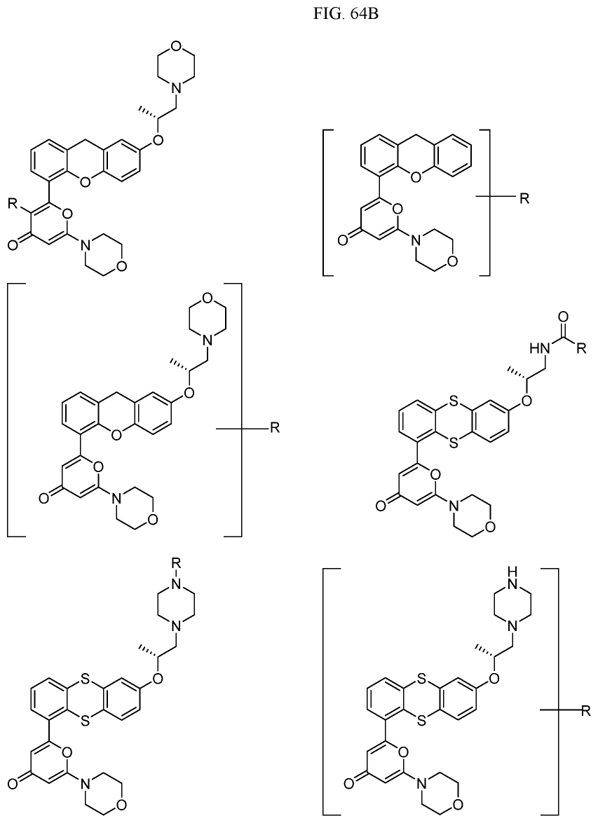

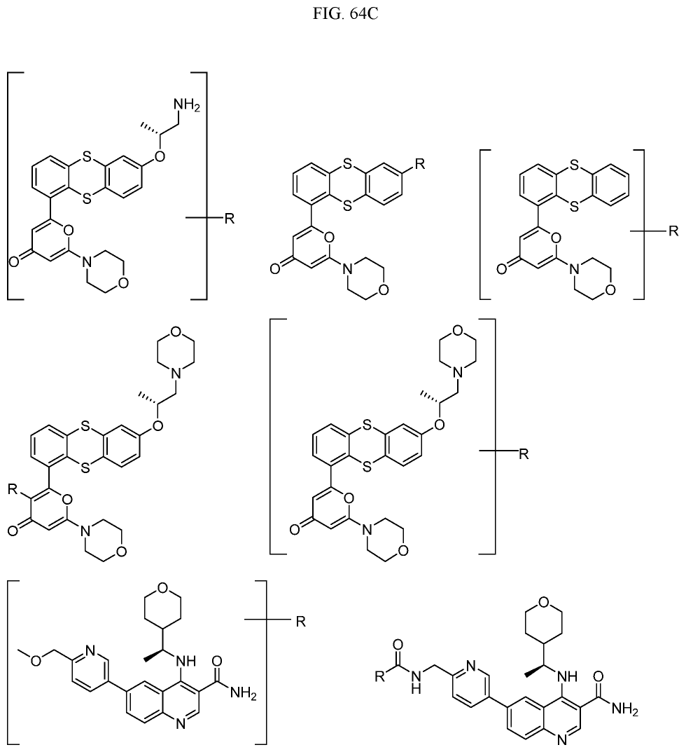

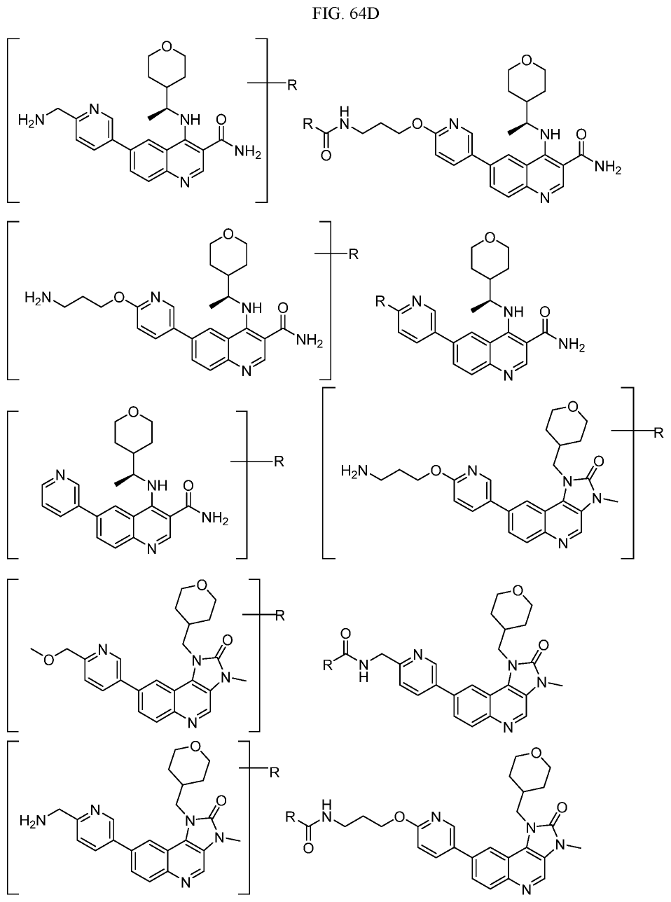

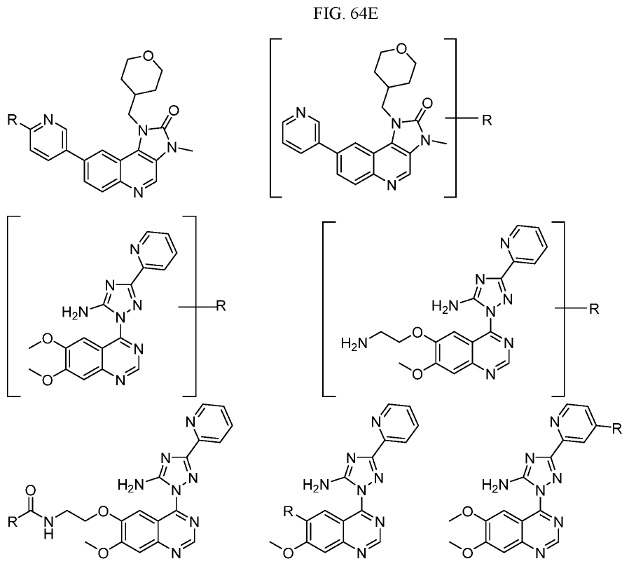

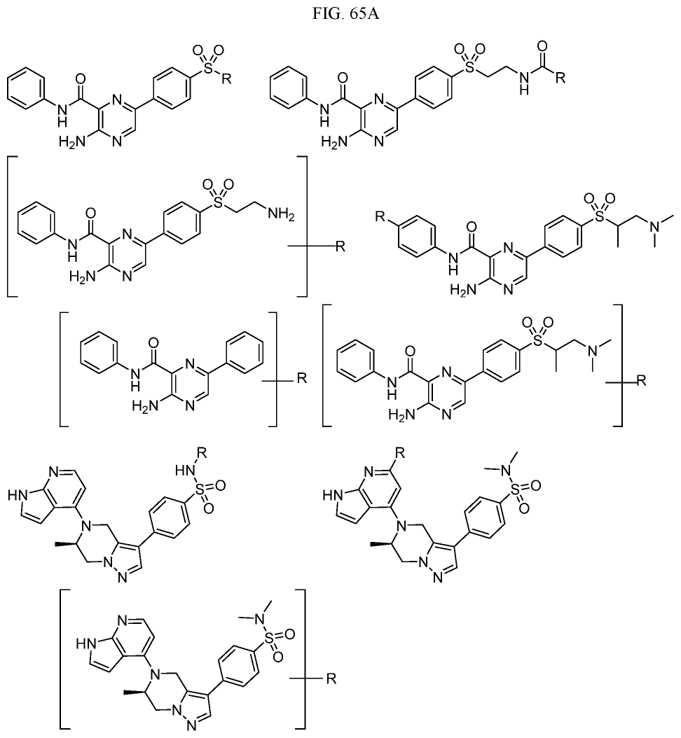

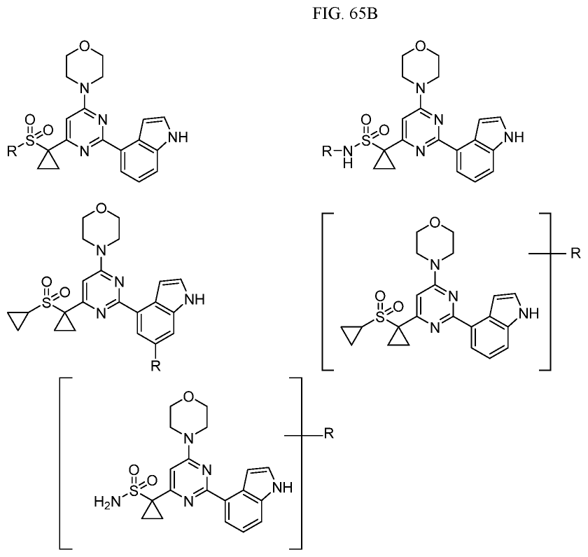

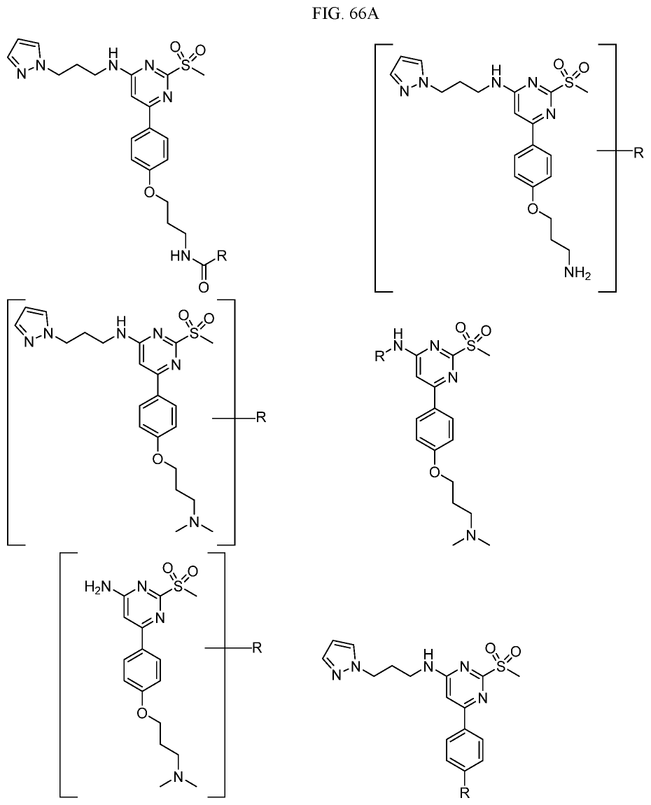

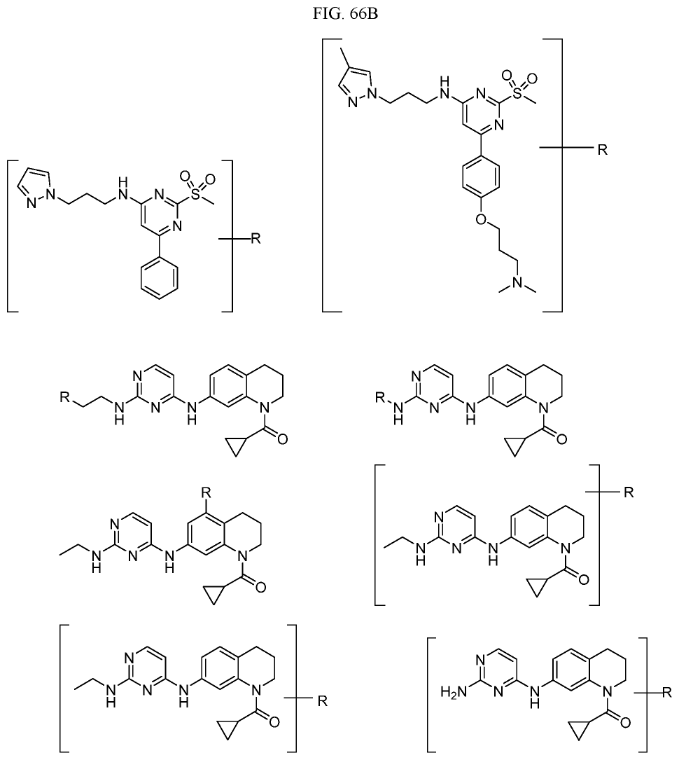

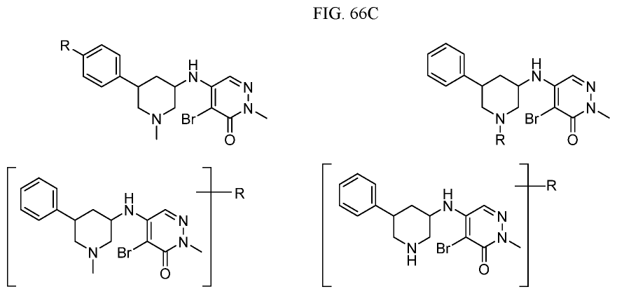

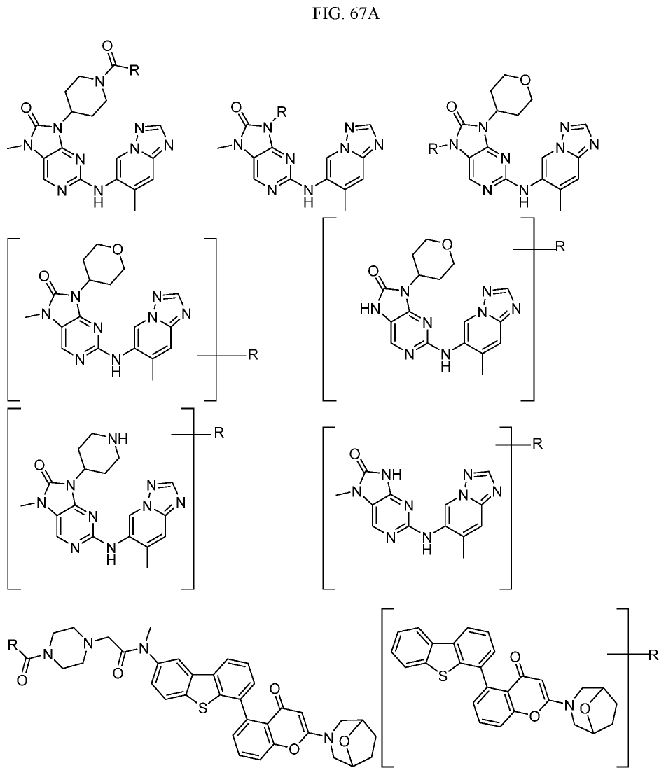

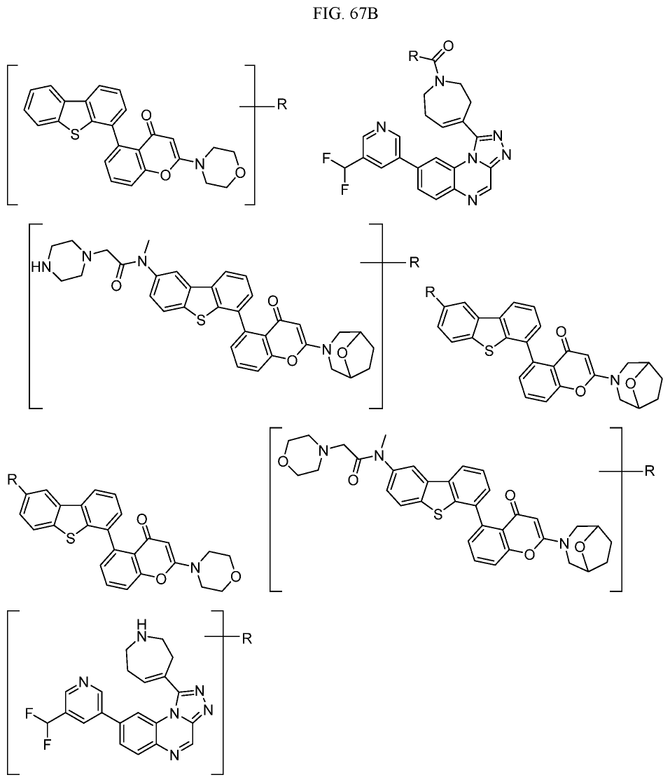

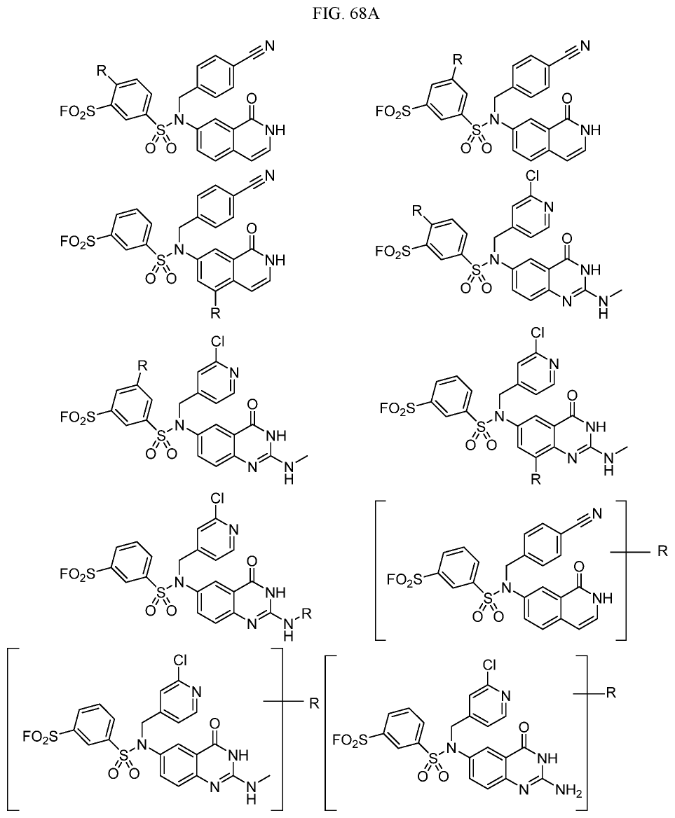

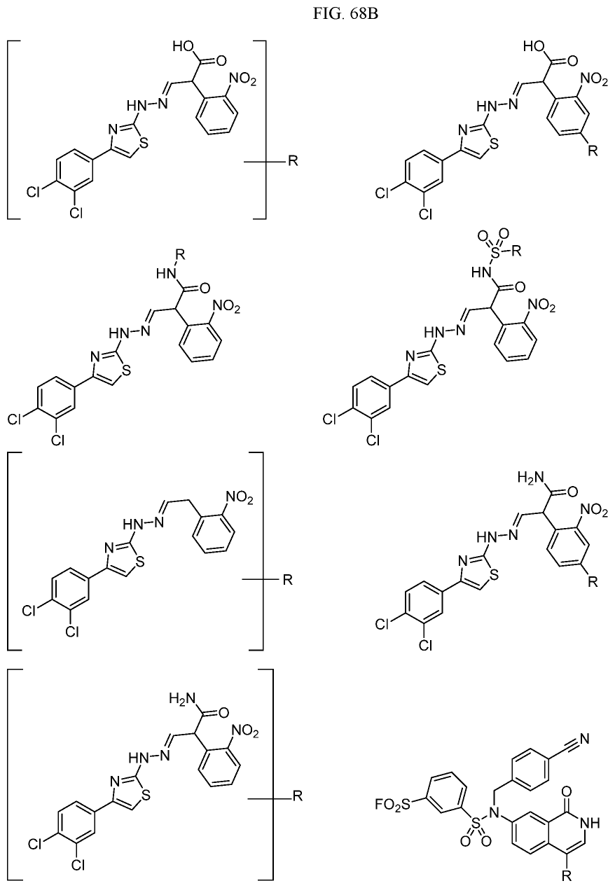

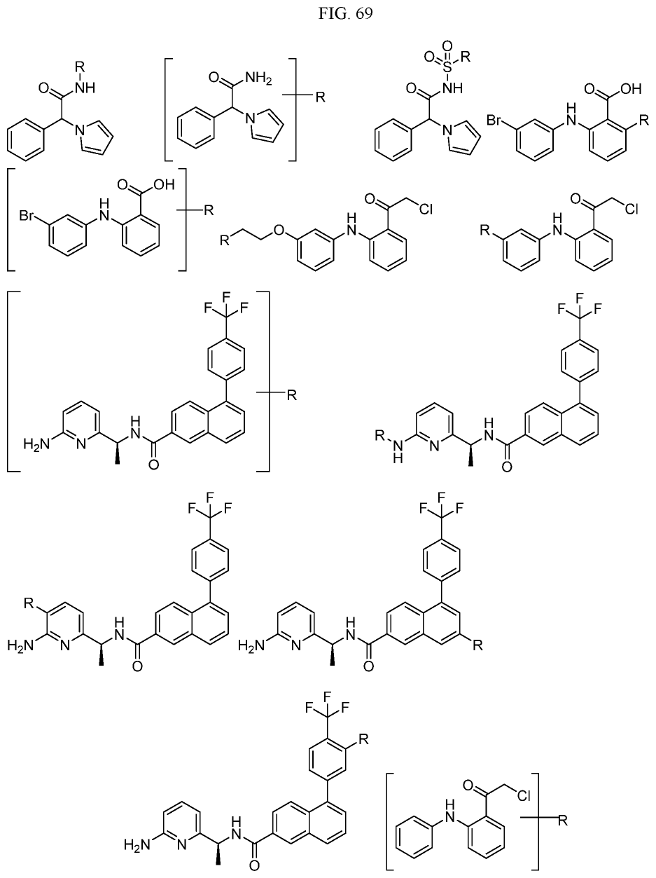

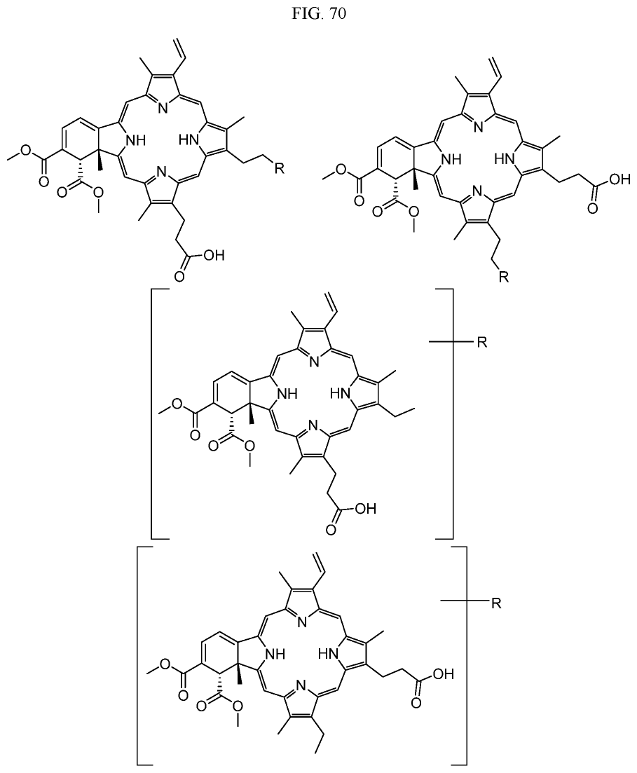

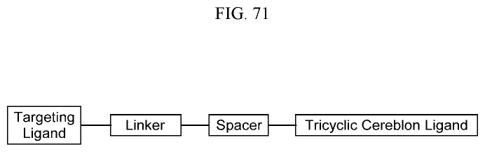

FIG. 30A and FIG. 30B provide non-limiting examples of GRB2 Targeting Ligands, wherein R represents exemplary points at which the spacer is attached. For additional examples, see the crystal structure PDB 1CJ1 (Furet, P., et al., “Structure-based design, synthesis, and X-ray crystallography of a high-affinity antagonist of the Grb2-SH2 domain containing an asparagine mimetic”, J. Med. Chem., 1999, 42: 2358-2363); the crystal structure PDB 2AOA, 2AOB (Phan, J., et al., “Crystal Structures of a High-affinity Macrocyclic Peptide Mimetic in Complex with the Grb2 SH2 Domain”, J Mol Biol., 2005, 353: 104-115); the crystal structure PDB 3KFJ, 31N7, 3IMJ, 3IMD, 3IN8 (Delorbe, J. E., et al., “Thermodynamic and Structural Effects of Conformational Constraints in Protein-Ligand Interactions. Entropic Paradoxy Associated with Ligand Preorganization.”, J Am Chem Soc., 2009, 131: 16758-16770); the crystal structure PDB 2HUW, 3C71 (Benfield, A. P., et al., “Ligand Preorganization May Be Accompanied by Entropic Penalties in Protein-Ligand Interactions.”, Angew Chem Int Ed Engl., 2006, 45: 6830-6835); and the crystal structure PDB 1X0N (Ogura, K et al., “NMR structure of growth factor receptor binding protein SH2 domain complexed with the inhibitor”, to be published).

FIG. 31 provides non-limiting examples of KMT2D or MLL2/MLL4Targeting Ligands, wherein R represents exemplary points at which the spacer is attached. For additional examples, see the crystal structure PDB 7BRE (Li, Y., et al., “Crystal Structure of MLL2 Complex Guides the Identification of a Methylation Site on P53 Catalyzed by KMT2 Family Methyltransferases.”, Structure, 2020); the crystal structure PDB 4ZAP (Zhang, Y., et al., “Evolving Catalytic Properties of the MLL Family SET Domain.”, Structure, 2015, 23: 1921-1933); the crystal structure PDB 6KIZ (Xue, H., et al., “Structural basis of nucleosome recognition and modification by MLL methyltransferases.”, Nature, 2019, 573: 445-449); and the crystal structures PDB 3UVK (Zhang, P., et al., “The plasticity of WDR5 peptide-binding cleft enables the binding of the SETi family of histone methyltransferases.”, Nucleic Acids Res., 2012, 40: 4237-4246).

FIG. 32 provides non-limiting examples of MLLT1 or ENL Targeting Ligands, wherein R represents exemplary points at which the spacer is attached. For additional examples, see the crystal structure PDB 6HT0, 6HT1 (Moustakin, M. et al., “Discovery of an MLLT1/3 YEATS Domain Chemical Probe”, Angew Chem Int Ed Engl., 2018, 57: 16302-16307); the crystal structures PDB 6T1I, 6T1J, 6TIL, 6T1M, 6T1N, 6T10 (Ni, X., et al., “Structural Insights into Interaction Mechanisms of Alternative Piperazine-urea YEATS Domain Binders in MLLT1”, ACS Med Chem Lett., 2019, 10: 1661-1666); and the crystal structures PDB 6HPW, 6HPY, 6HPX,6HPZ (Heidenreich, D., et al., “Structure-Based Approach toward Identification of Inhibitory Fragments for Eleven-Nineteen-Leukemia Protein (ENL)”, J. Med. Chem., 2018, 61: 10929-10934).

FIG. 33 provides non-limiting examples of NSD3 Targeting Ligands, wherein R represents exemplary points at which the spacer is attached. For additional examples, see the crystal structure PDB 6G24, 6G25, 6G29, 6G2B, 6G2C, 6G2E, 6G2F, 6G20, 6G3T (Bottcher, J., et al., “Fragment-based discovery of a chemical probe for the PWWP1 domain of NSD3”, Nat Chem Biol., 2019, 15: 822-829); the crystal structure PDB 5UPD (Tempel, W., et al., “Methyltransferase domain of human Wolf-Hirschhorn Syndrome Candidate 1-Like protein 1 (WHSC1L1)”, to be published); and the crystal structure PDB 6CEN (Morrison, M. J., et al., “Identification of a peptide inhibitor for the histone methyltransferase WHSC1”, PLoS One, 2018, 13: e0197082-e0197082).

FIG. 34 provides non-limiting examples of PPM1D or WIP1 Targeting Ligands, wherein R represents exemplary points at which the spacer is attached. For additional examples, see the crystal structure PDB 3UYH, ADA3, 4DAQ (Micco, M., et al., “Structure-based design and evaluation of naphthalene diimide g-quadruplex ligands as telomere targeting agents in pancreatic cancer cells”, J. Med. Chem., 2013, 56: 2959-2974).

FIG. 35A-35B provide non-limiting examples of SOS1 Targeting Ligands, wherein R represents exemplary points at which the spacer is attached. For additional examples, see the crystal structure PDB 50VE, 50VF, 50VG, 50VH, 50VI, (Hillig, R. C., et al., “Discovery of potent SOS1 inhibitors that block RAS activation via disruption of the RAS-SOS1 interaction”, Proc Natl Acad Sci USA., 2019, 116: 2551-2560); the crystal structure PDB 6F08 (Ballone, A., et al., “Structural characterization of 14-3-3 zeta in complex with the human Son of sevenless homolog 1 (SOS1)”, J Struct Biol., 2018, 202: 210-215); the crystal structure PDB 6D5E, 6D5G, 6D5H, 6D5J, 6D5L, 6D5M, 6D5V, 6D5W, 6D55, 6D59, (Hodges, T. R. et al., “Discovery and Structure-Based Optimization of Benzimidazole-Derived Activators of SOS1-Mediated Nucleotide Exchange on RAS”, J. Med. Chem., 2018, 61: 8875-8894); the crystal structure PDB 6SCM, 6SFR (Kessler, D., et al., “SOS1 in Complex with Inhibitor BI-3406”, to be published); the crystal structure PDB 6V94, 6V9J, 6V9L, 6V9M, 6V9N (Sarkar, D., et al., “Discovery of Sulfonamide-Derived Agonists of SOS1-Mediated Nucleotide Exchange on RAS Using Fragment-Based Methods.”, J. Med. Chem., 2020, 63: 8325-8337).

FIG. 36 provides non-limiting examples of TBXT or Brachyury Targeting Ligands, wherein R represents exemplary points at which the spacer is attached. For additional examples, see the crystal structure PDB 5QS6, 5QSC, 5QSE, 5QSF, 5QRW, (Newman, J. A., et al., “PanDDA analysis group deposition”, to be published); and the crystal structure PBD 6ZU8 (Newman, J. A., et al., “Crystal structure of human Brachyury G177D variant in complex with Afatinib”, to be published).

FIG. 37A-37C provide non-limiting examples of USP7 Targeting Ligands, wherein R represents exemplary points at which the spacer is attached. For additional examples, see the crystal structure PDB 5UQV, 5UQX (Kategaya, L., et al., “USP7 small-molecule inhibitors interfere with ubiquitin binding”, Nature, 2017, 550: 534-538); the crystal structures PDB 6VN2, 6VN3, 6VN4, 6VN5, 6VN6 (Leger, P. R., et al., “Discovery of Potent, Selective, and Orally Bioavailable Inhibitors of USP7 with In Vivo Antitumor Activity.”, J. Med. Chem., 2020, 63: 5398-5420); and the crystal structures PDB 5N9R, 5N9T (Gavory, G., et al., “Discovery and characterization of highly potent and selective allosteric USP7 inhibitors.”, Nat Chem Biol., 2018, 14: 118-125); and the crystal structure PDB 5NGE, 5NGF (Turnbull, A. P., et al., “Molecular basis of USP7 inhibition by selective small-molecule inhibitors”, Nature, 2017, 550: 481-486).

FIG. 38 provides non-limiting examples of BKV and JCV Targeting Ligands, wherein R represents exemplary points at which the spacer is attached. For additional examples, see the crystal structure PDB 5J4V, 5J4Y (Bonafoux, D., et al., “Fragment-Based Discovery of Dual JC Virus and BK Virus Helicase Inhibitors.”, J. Med. Chem., 2016, 59: 7138-7151).

FIG. 39 provides non-limiting examples of CK1α (Casein kinase 1 alpha) Targeting Ligands, wherein R represents exemplary points at which the spacer is attached. For additional examples, see the crystal structure PDB 5ML5, 5MQV (Halekotte, J., et al., “Optimized 4,5-Diarylimidazoles as Potent/Selective Inhibitors of Protein Kinase CK1 delta and Their Structural Relation to p38 alpha MAPK.”, Molecules, 2017,22).

FIG. 40 provides non-limiting examples of GSPT1/ERF3 Targeting Ligands, wherein R represents exemplary points at which the spacer is attached. For additional examples, see the crystal structure PDB 5LZT, 5LZS, 5LZV, 5LZU, 5LZX, 5LZW, 5LZZ, 5LZY (Shao, S., et al., “Decoding Mammalian Ribosome-mRNA States by Translational GTPase Complexes”, Cell, 2016, 167: 1229-1240.e15).

FIG. 41 provides non-limiting examples of IFZV Targeting Ligands, wherein R represents exemplary points at which the spacer is attached. For additional examples, see the crystal structure PDB (Iyer, S., et al., “The crystal structure of human placenta growth factor-1 (PlGF-1), an angiogenic protein, at 2.0 A resolution.”, J Biol Chem., 2001, 276: 12153-12161); and the crystal structure PDB IRV6 (Christinger, H. W., et al., “The crystal structure of placental growth factor in complex with domain 2 of vascular endothelial growth factor receptor-1”, J Biol Chem., 2004, 279: 10382-10388).

FIG. 42 provides non-limiting examples of NSD2 Targeting Ligands, wherein R represents exemplary points at which the spacer is attached. For additional examples, see the crystal structure PDB 6XCG (Zhou, M. Q., “Histone-lysine N-methyltransferase NSD2-PWWP1 with compound UNC6934”, to be published); and the crystal structure PDB 6UE6 (Liu, Y., et al., “PWWP1 domain of NSD2 in complex with MR837”, to be published).

FIG. 43 provides non-limiting examples of TAU Targeting Ligands, wherein R represents exemplary points at which the spacer is attached. For additional examples, see the crystal structure PDB 6VA2, 6VA3 (Chen, J. L. et al., “Design, Optimization, and Study of Small Molecules That Target Tau Pre-mRNA and Affect Splicing.”, J Am Chem Soc., 2020, 142: 8706-8727).

FIG. 44 provides non-limiting examples of CYP17A1 Targeting Ligands, wherein R represents exemplary points at which the spacer is attached. For additional examples, see the crystal structure PDB 3RUK, 3SWZ (Devore, N. M. et al., “Structures of cytochrome P450 17A1 with prostate cancer drugs abiraterone and TOK-001”, Nature, 2012, 482: 116-119); and the crystal structure PDB 6CHI, 6CIZ, (Fehl, C., et al., “Structure-Based Design of Inhibitors with Improved Selectivity for Steroidogenic Cytochrome P450 17A1 over Cytochrome P450 21A2”, J Med Chem., 2018, 61: 4946-4960).

FIG. 45 provides non-limiting examples SALL4 Targeting Ligands, wherein R represents exemplary points at which the spacer is attached. For additional examples, see the crystal structure PDB 7BQU, 7BQV (Furihata, H., et al., “Structural bases of IMiD selectivity that emerges by 5-hydroxythalidomide”, Nat Commun., 2020, 11: 4578-4578); and the crystal structure PDB 6UML (Matyskiela, M. E., et al., “Crystal structure of the SALL4-pomalidomide-cereblon-DDB1 complex”, Nat Struct Mol Biol., 2020, 27: 319-322).

FIG. 46 provides non-limiting examples of FAM38 Targeting Ligands, wherein R represents exemplary points at which the spacer is attached. For additional examples, see the crystal structure PDB 6KG7 (Wang, L., et al., “Structure and mechanogating of the mammalian tactile channel PIEZO2.”, Nature, 2019, 573: 225-229).

FIG. 47 provides non-limiting examples of CYP20A1 Targeting Ligands, wherein R represents exemplary points at which the spacer is attached. For additional examples, see Durairaj et al. Biological Chemistry, 2020, 401(3), 361-365.

FIG. 48 provides non-limiting examples of HTT Targeting Ligands, wherein R represents exemplary points at which the spacer is attached. For additional examples, see the crystal structure PDB 5XI1 (Khan, E., et al., “Myricetin Reduces Toxic Level of CAG Repeats RNA in Huntington's Disease (HD) and Spino Cerebellar Ataxia (SCAs).”, ACS Chem Biol., 2018, 13: 180-188).

FIG. 49 provides non-limiting examples of KRAS Targeting Ligands, wherein R represents exemplary points at which the spacer is attached. For additional examples, see the crystal structure PDB 6CU6 (Hobbs, G. A., et al., “Atypical KRASG12RMutant Is Impaired in PI3K Signaling and Macropinocytosis in Pancreatic Cancer.”, Cancer Discov., 2020, 10: 104-123); the crystal structure PDB 6GJ5, 6GJ6, 6GJ8, 6JG7, (“Drugging an Undruggable Pocket on KRAS” PNAS 2019 116 (32) 15823-15829); and the crystal structure PDB 6BP1 (Lu, J., et al., “KRAS Switch Mutants D33E and A59G Crystallize in the State 1 Conformation.”, Biochemistry, 2018, 57: 324-333).

FIG. 50 provides non-limiting examples of NRF2 (NFE2L2) Targeting Ligands, wherein R represents exemplary points at which the spacer is attached. For additional examples, see the crystal structure PDB 5CGJ (Winkel, A. F., et al., “Characterization of RA839, a Noncovalent Small Molecule Binder to Keap1 and Selective Activator of Nrf2 Signaling.”, J Biol Chem., 2015, 290: 28446-28455); and 6TYM, 6TYP (Ma, B., et al., “Design, synthesis and identification of novel, orally bioavailable non-covalent Nrf2 activators”, Bioorg Med Chem Lett., 2020, 30: 126852-126852).

FIG. 51 provides non-limiting examples of P300 Targeting Ligands, wherein R represents exemplary points at which the spacer is attached. For additional examples, see the crystal structure PDB 4PZR, 4PZS, 4PZT (Maksimoska, J., et al., “Structure of the p300 Histone Acetyltransferase Bound to Acetyl-Coenzyme A and Its Analogues”, Biochemistry, 2014, 53: 3415-3422); and the crystal structure PDB 6PGU (Gardberg, A. S., et al., “Make the right measurement: Discovery of an allosteric inhibition site for p300-HAT”, Struct Dyn., 2019, 6: 054702-054702).

FIG. 52 provides non-limiting examples of PIK3CA Targeting Ligands, wherein R represents exemplary points at which the spacer is attached. For additional examples, see the crystal structure PDB 60AC (Rageot, D., et al., “(S)-4-(Difluoromethyl)-5-(4-(3-methylmorpholino)-6-morpholino-1,3,5-triazin-2-yl)pyridin-2-amine (PQR530), a Potent, Orally Bioavailable, and Brain-Penetrable Dual Inhibitor of Class I PI3K and mTOR Kinase”, J Med Chem., 2019, 62: 6241-6261); and the crystal structure PDB 5SX8, 5SWP (Miller, M. S. et al., “Identification of allosteric binding sites for PI3K alpha oncogenic mutant specific inhibitor design.”, Bioorg Med Chem., 2017, 25: 1481-1486).

FIG. 53 provides non-limiting examples of SARM1 Targeting Ligands, wherein R represents exemplary points at which the spacer is attached. For additional examples, see the crystal structure PDB 6QWV (Sporny, M., et al., “Structural Evidence for an Octameric Ring Arrangement of SARM1”, J Mol Biol, 2019, 431: 3591-3605); and the crystal structure PDB 600Q, 600R, 600T, 600V, 600W (Horsefield, S., et al., “NALD+ cleavage activity by animal and plant TIR domains in cell death pathways”, Science, 2019, 365: 793-799).

FIG. 54 provides non-limiting examples of SNCA Targeting Ligands, wherein R represents exemplary points at which the spacer is attached. For additional examples, see the crystal structure PDB 415M, 415P, 41613, 416F, 416H (Aubele, D L., et al., “Selective and brain-permeable polo-like kinase-2 (Plk-2) inhibitors that reduce alpha-synuclein phosphorylation in rat brain”, Chem Med Chem., 2013, 8: 1295-1313).

FIG. 55 provides non-limiting examples of MAPT Targeting Ligands, wherein R represents exemplary points at which the spacer is attached. For example, the crystal structure PDB 6VI3, 6VHL (Arakhamia, T., et al., “Posttranslational Modifications Mediate the Structural Diversity of Tauopathy Strains”, Cell, 2020, 180: 633-644.e12); and the crystal structure PDB 6FAU, 6FAV, 6FAW, 6FBW, 6FBY, 6FI4, 6FI5 (Andrei, S. A., et al., “Inhibition of 14-3-3/Tau by Hybrid Small-Molecule Peptides Operating via Two Different Binding Modes.”, ACS Chem Neurosci., 2018, 9: 2639-2654).

FIG. 56 provides non-limiting examples of PTPN2 or TCPTP Targeting Ligands, wherein R represents exemplary points at which the spacer is attached. For example, the crystal structure PDB 2FJN, 2FJM (Asante-Appiah, E., et al., “Conformation-assisted inhibition of protein-tyrosine phosphatase-1B elicits inhibitor selectivity over T-cell protein-tyrosine phosphatase”, J Biol Chem., 2006, 281: 8010-8015).

FIG. 57 provides non-limiting examples of STAT3 Targeting Ligands, wherein R represents exemplary points at which the spacer is attached. The examples shown here derive from compounds in Zheng, W. et al. MMPP Attenuates Non-Small Cell Lung Cancer Growth by Inhibiting the STAT3 DNA-Binding Activity via Direct Binding to the STAT3 DNA-Binding Domain, Theranostics 2017, 7(18):4632 and US2006/0247318. For additional examples, see Yang, L. et al. Novel Activators and Small-Molecule Inhibitors of STAT3 in Cancer, Cytokine & Growth Factor Reviews 2019, 49, 10-22.

FIG. 58 provides non-limiting examples of MyD88 Targeting Ligands, wherein R represents exemplary points at which the spacer is attached. The examples shown here derive from compounds in Sucking, C. et al Small Molecule Analogues of the parasitic worm product ES-62 interact with the TIR domain of MyD88 to inhibit pro-inflammatory signaling (2018) 8:2123 and Loiarro, M. et al Pivotal Advance: Inhibition of MyD88 dimerization and recruitment of IRAK1 and IRAK4 by a novel peptidomimetic compound. Journal of Leukocyte Biology, (2007) 82: 801-810.

FIG. 59 provides non-limiting examples of PTP4A3 Targeting Ligands, wherein R represents exemplary points at which the spacer is attached. The examples shown here derive from compounds in Ahn, J. et al Synthesis and Biological Evaluation of RhodanineD derivatives as PRL-3 Inhibitors Bioorganic & Medicinal Chemistry Letters (2006) 16(11):2996-2999 and Min, G. et al Rhodanine-Based PRL-3 Inhibitors Blocked the Migration and Invasion of Metastatic Cancer Cells Bioorganic & Medicinal Chemistry Letters (2013) 23(13):3769-3774. For additional examples, see Tasker, N. et al Tapping the Therapeutic Potential of Protein Tyrosine Phosphatase 4A with Small Molecule Inhibitors Bioorganic & Medicinal Chemistry Letters (2019) 29(16):2008-2015.

FIG. 60 provides non-limiting examples of SF3B1 Targeting Ligands, wherein R represents exemplary points at which the spacer is attached. The examples shown here derive from compounds in Kaida, D. et al Spliceostatin A Targets SF3b and Inhibits Both Splicing and Nuclear Retention of pre-mRNA Nature Chemical Biology (2007) 3:576-583 and Kotake, Y. et al Splicing Factor SF3b as a Target of the Antitumor Natural Product Pladienolide Nature Chemical Biology (2007) 3:570-575. For additional examples, see Effenberger, K. et al Modulating Splicing with Small Molecular Inhibitors of the Spliceosome WIREs RNA (2016) 8:e1381.

FIG. 61 provides non-limiting examples of ARID1B and ARID2 Targeting Ligands, wherein R represents exemplary points at which the spacer is attached. For additional examples, see Chory et al. ACS Chemical Biology 2020, 15(6), 1685.

FIG. 62 provides non-limiting examples of Class II BRAF Mutant Targeting Ligands, wherein R represents exemplary points at which the spacer is attached. For additional examples, see Cho et al. Biochemical and Biophysical Research Communications 2020, 352(2), 315.

FIG. 63 provides non-limiting examples of NRAS^{Q61K} Targeting Ligands, wherein R represents exemplary points at which the spacer is attached. For additional examples, see Song et al. Am J Cancer Res 2017, 7(4), 831 and Johnson et al. Curr Treat Options Oncol. 2015, 16(4), 15.

FIGS. 64A-64E provide non-limiting examples of ataxia telangiectasia-mutated (ATM) kinase targeting Ligands wherein R represents exemplary points at which the linker is attached.

Additional examples are provided in J Med Chem, 2019, 62: 2988-3008.

FIGS. 65A-65B provide non-limiting examples of ATR Targeting Ligands wherein R represents exemplary points at which the linker is attached. Additional examples are provided in Journal of Molecular Biology Volume 429, Issue 11, 2 Jun. 2017, Pages 1684-1704.

FIGS. 66A-66C provide non-limiting examples of BPTF targeting ligands wherein R represents exemplary points at which the linker is attached. Additional examples are provided in Organic & Biomolecular Chemistry 2020, 18(27): 5174-5182.

FIGS. 67A-67B provide non-limiting examples of DNA-PK targeting ligands wherein R represents exemplary points at which the linker is attached. Additional examples are provided in J. Med. Chem. 2020, 63, 7, 3461-3471.

FIGS. 68A-68B provide non-limiting examples of elf4E Targeting Ligands wherein R represents exemplary points at which the linker is attached. Additional examples are provided in J. Am. Chem. Soc. 2020, 142, 4960-4964.

FIG. 69 provides non-limiting examples of TEAD, for example, TEAD1, TEAD2, TEAD3, and/or TEAD4 targeting ligands wherein R represents exemplary points at which the linker is attached.

FIG. 70 provides non-limiting examples of YAP targeting ligands wherein R represents exemplary points at which the linker is attached.

FIG. 71 provides a non-limiting representative formula of Target Protein degrading compounds of the present invention.

### DETAILED DESCRIPTION OF THE INVENTION

Compounds and their uses and manufacture are provided that degrade a disease-mediating Target Protein via the ubiquitin proteasome pathway (UPP) and thus are useful to treat a disorder responsive to degradation by the protein. The invention provides compounds of general Formula I, Formula II, or Formula III or a pharmaceutically acceptable salt thereof that include a Targeting Ligand that binds to a Target Protein, an E3 Ligase binding portion (Tricyclic Cereblon Ligand), a Linker that covalently links the Targeting Ligand to a Spacer, and a Spacer that covalently links the Linker to the E3 Ligase binding portion.

A compound of the present invention provided herein or its pharmaceutically acceptable salt and/or its pharmaceutically acceptable composition can be used to treat a disorder which is mediated by a Target Protein. The Target Protein is typically a mutated, altered or overexpressed protein wherein the mutation, alteration or overexpression converts its normal function into a dysfunction which causes or contributes to disease. In some aspects, the disease is an abnormal cellular proliferation such as cancer or a tumor. In some embodiments a method to treat a patient with a disorder mediated by a Target Protein is provided that includes administering an effective amount of one or more compounds as described herein, or a pharmaceutically acceptable salt thereof, to the patient, typically a human, optionally in a pharmaceutically acceptable composition.

The tricyclic heterobifunctional compounds provided herein are catalytic. The Target Protein degradation mediated by the compound typically occurs rapidly, on the order of milliseconds from initial target-ligase encounter to poly-ubiquitination and release for degradation by the proteasome. Once the targeted protein degradation process occurs for one molecule of a target protein, the degrader is released and the process is repeated with the same degrader molecule. This recursive process of binding the target protein, ternary complex formation with the E3 ligase, ubiquitination and release for degradation can occur thousands of times with a single degrader molecule.

### I. Definitions

Unless otherwise defined, all technical and scientific terms used herein have the same meaning as commonly understood by one of ordinary skill in the art to which this application belongs. In the specification, singular forms also include the plural unless the context clearly dictates otherwise. Although methods and materials similar or equivalent to those described herein can be used in the practice and testing of the present application, suitable methods and materials are described below. All publications, patent applications, patents, and other references mentioned herein are incorporated by reference. The references cited herein are not admitted to be prior art to the claimed application. In the case of conflict, the present specification, including definitions, will control. In addition, the materials, methods, and examples are illustrative only and are not intended to be limiting.

Compounds are described using standard nomenclature. Unless defined otherwise, all technical and scientific terms used herein have the same meaning as is commonly understood by one of skill in the art to which this invention belongs.

In certain embodiments of each compound described herein, the compound may be in the form of a racemate, enantiomer, mixture of enantiomers, diastereomer, mixture of diastereomers, tautomer, N-oxide, or isomer, such as a rotamer, as if each is specifically described unless specifically excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed.

The present invention includes compounds described herein with at least one desired isotopic substitution of an atom, at an amount above the natural abundance of the isotope, i.e., enriched. Isotopes are atoms having the same atomic number but different mass numbers, i.e., the same number of protons but a different number of neutrons. If isotopic substitutions are used, the common replacement is at least one deuterium for hydrogen.

More generally, examples of isotopes that can be incorporated into compounds of the invention include isotopes of hydrogen, carbon, nitrogen, oxygen, fluorine, and chlorine such as ²H, ³H, ¹¹C, ¹³C, ¹⁴C, ¹⁵N, ¹⁷O, ¹⁸O, ¹⁸F, ³⁵S, and ³⁶Cl respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Additionally, any hydrogen atom present in the compound of the invention may be substituted with an ¹⁸F atom, a substitution that may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

By way of general example and without limitation, isotopes of hydrogen, for example, deuterium (2H) and tritium (3H) may be used anywhere in described structures that achieves the desired result. Alternatively or in addition, isotopes of carbon, e.g., ¹³C and ¹⁴C, may be used.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In one non-limiting embodiment, the substitution of a hydrogen atom for a deuterium atom can be provided in any compound described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated. In certain embodiments, at least one deuterium is placed on an atom that has a bond which is broken during metabolism of the compound in vivo, or is one, two or three atoms remote form the metabolized bond (e.g., which may be referred to as an α, β or γ, or primary, secondary or tertiary isotope effect).

The compounds of the present invention may form a solvate with a solvent (including water). Therefore, in one non-limiting embodiment, the invention includes a solvated form of the compounds described herein. The term “solvate” refers to a molecular complex of a compound of the present invention (including a salt thereof) with one or more solvent molecules. Non-limiting examples of solvents are water, ethanol, isopropanol, dimethyl sulfoxide, acetone and other common organic solvents. The term “hydrate” refers to a molecular complex comprising a compound of the invention and water. Pharmaceutically acceptable solvates in accordance with the invention include those wherein the solvent may be isotopically substituted, e.g. D₂O, d₆-acetone, d₆-DMSO. A solvate can be in a liquid or solid form.

A dash (“-”) that is not between two letters or symbols is used to indicate a point of attachment for a substituent. For example, —(C═O)NH₂ is attached through carbon of the keto (C═O) group.

“Alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. Unless otherwise indicated, the term alkyl includes cycloalkyl or carbocycle. “Alkenyl” is a linear or branched aliphatic hydrocarbon groups having one or more carbon-carbon double bonds that may occur at a stable point along the chain. The specified ranges as used herein indicate an alkenyl group having each member of the range described as an independent species, as described above for the alkyl moiety. In one non-limiting embodiment, the alkenyl contains from 2 to about 12 carbon atoms, more generally from 2 to about 6 carbon atoms or from 2 to about 4 carbon atoms. In certain embodiments the alkenyl is C2, C2-C3, C₂-C₄, C₂-C₅, or C₂-C₆. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. The term “Alkenyl” also encompasses cycloalkyl or carbocyclic groups possessing at least one point of unsaturation. “Alkynyl” is a branched or straight chain aliphatic hydrocarbon group having one or more carbon-carbon triple bonds that may occur at any stable point along the chain. The specified ranges as used herein indicate an alkynyl group having each member of the range described as an independent species, as described above for the alkyl moiety. In one non-limiting embodiment, the alkynyl contains from 2 to about 12 carbon atoms, more generally from 2 to about 6 carbon atoms or from 2 to about 4 carbon atoms. In certain embodiments the alkynyl is C2, C2-C3, C2-C4, C2-C5, or C2-C6. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. The term “Alkynyl” also encompasses cycloalkyl or carbocyclic groups possessing at least one point of triple bond unsaturation. “Halo” and “Halogen” is independently fluorine, chlorine, bromine or iodine. “Haloalkyl” is a branched or straight-chain alkyl groups substituted with 1 or more halo atoms described above, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include but are not limited to, trifluoromethyl and pentafluoroethyl.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more cycloalkyl or heterocycle groups wherein the radical or point of attachment is on the aryl ring, and in such instances, the number of carbon atoms continue to designate the number of carbon atoms in the aryl ring system. The one or more fused cycloalkyl or heterocycle groups can be a 4 to 7-membered saturated or partially unsaturated cycloalkyl or heterocycle groups. “Arylalkyl” refers to either an alkyl group as defined herein substituted with an aryl group as defined herein or to an aryl group as defined herein substituted with an alkyl group as defined herein.

The term “heterocycle” denotes saturated and partially saturated heteroatom-containing ring radicals, wherein there are 1, 2, 3, or 4 heteroatoms independently selected from nitrogen, sulfur, boron, silicone, and oxygen. Heterocyclic rings may comprise monocyclic 3-10 membered rings, as well as 5-16 membered bicyclic ring systems (which can include bridged, fused, and spiro—fused bicyclic ring systems). It does not include rings containing —O—O—, —O—S—or —S—S— portions. Examples of saturated heterocycle groups include saturated 3- to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl.

“Heterocycle” also includes groups wherein the heterocyclic radical is fused/condensed with an aryl or carbocycle radical, wherein the point of attachment is the heterocycle ring. “Heterocycle” also includes groups wherein the heterocyclic radical is substituted with an oxo group

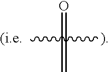 [SMILES: *=O]

For example a partially unsaturated condensed heterocyclic group containing 1 to 5 nitrogen atoms, for example, indoline or isoindoline; a partially unsaturated condensed heterocyclic group containing 1 to 2 oxygen atoms and 1 to 3 nitrogen atoms; a partially unsaturated condensed heterocyclic group containing 1 to 2 sulfur atoms and 1 to 3 nitrogen atoms; and a saturated condensed heterocyclic group containing 1 to 2 oxygen or sulfur atoms.

The term “heterocycle” also includes “bicyclic heterocycle”. The term “bicyclic heterocycle” denotes a heterocycle as defined herein wherein there is one bridged, fused, or spirocyclic portion of the heterocycle. The bridged, fused, or spirocyclic portion of the heterocycle can be a carbocycle, heterocycle, or aryl group as long as a stable molecule results. Unless excluded by context the term “heterocycle” includes bicyclic heterocycles. Bicyclic heterocycle includes groups wherein the fused heterocycle is substituted with an oxo group. Non-limiting examples of bicyclic heterocycles include:

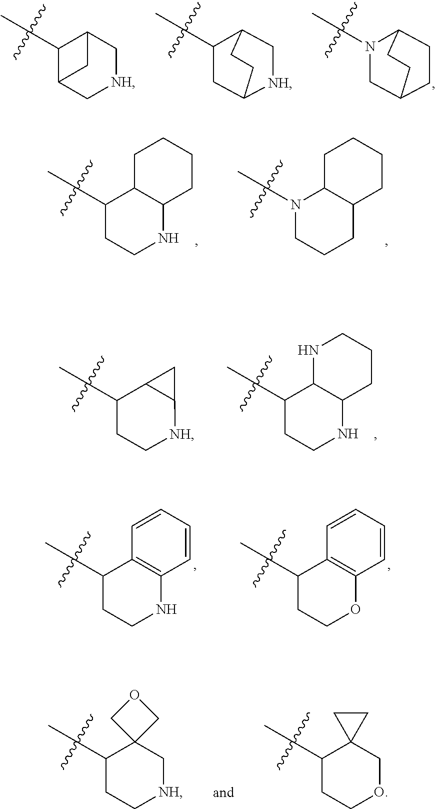

“Heterocyclealkyl” refers to either an alkyl group as defined herein substituted with a heterocycle group as defined herein or to a heterocycle group as defined herein substituted with an alkyl group as defined herein.

The term “heteroaryl” denotes stable aromatic ring systems that contain 1, 2, 3, or 4 heteroatoms independently selected from O, N, and S, wherein the ring nitrogen and sulfur atom(s) are optionally oxidized, and nitrogen atom(s) are optionally quarternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, IH-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]. In certain embodiments the “heteroaryl” group is a 8, 9, or 10 membered bicyclic ring system. Examples of 8, 9, or 10 membered bicyclic heteroaryl groups include benzofurazanyl, benzothiophenyl, benzothiazolyl, benzoxazolyl, quinazolinyl, quinoxalinyl, naphthyridinyl, quinolinyl, isoquinolinyl, benzofuranyl, indolyl, indazolyl, and benzotriazolyl. “Heteroarylalkyl” refers to either an alkyl group as defined herein substituted with a heteroaryl group as defined herein or to a heteroaryl group as defined herein substituted with an alkyl group as defined herein.

As used herein, “carbocyclic”, “carbocycle” or “cycloalkyl” includes a saturated or partially unsaturated (i.e., not aromatic) group containing all carbon ring atoms and from 3 to 14 ring carbon atoms (“C₃₋₁₄ cycloalkyl”) and zero heteroatoms in the non-aromatic ring system. In some embodiments, a cycloalkyl group has 3 to 10 ring carbon atoms (“C₃₋₁₀ cycloalkyl”). In some embodiments, a cycloalkyl group has 3 to 9 ring carbon atoms (“C₃₋₉ cycloalkyl”). In some embodiments, a cycloalkyl group has 3 to 8 ring carbon atoms (“C₃-s cycloalkyl”). In some embodiments, a cycloalkyl group has 3 to 7 ring carbon atoms (“C₃₋₇ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C5-10 cycloalkyl”). Exemplary C3-6 cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C3-6 cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), and the like. Exemplary C3-10 cycloalkyl groups include, without limitation, the aforementioned C3-s cycloalkyl groups as well as cyclononyl (C₉), cyclononenyl (C₉), cyclodecyl (C₁₀), cyclodecenyl (C₁₀), and the like. As the foregoing examples illustrate, in certain embodiments, the cycloalkyl group can be saturated or can contain one or more carbon-carbon double bonds. The term “cycloalkyl” also includes ring systems wherein the cycloalkyl ring, as defined above, is fused with one heterocycle, aryl or heteroaryl ring wherein the point of attachment is on the cycloalkyl ring, and in such instances, the number of carbons continue to designate the number of carbons in the carbocyclic ring system. The term “cycloalkyl” also includes ring systems wherein the cycloalkyl ring, as defined above, has a spirocyclic heterocycle, aryl or heteroaryl ring wherein the point of attachment is on the cycloalkyl ring, and in such instances, the number of carbons continue to designate the number of carbons in the carbocyclic ring system. The term “cycloalkyl” also includes bicyclic or polycyclic fused, bridged, or spiro ring systems that contain from 5 to 14 carbon atoms and zero heteroatoms in the non-aromatic ring system. Representative examples of “cycloalkyl” include, but are not limited to,

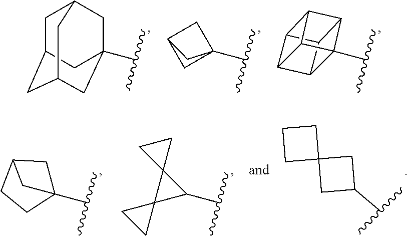

The term “bicycle” refers to a ring system wherein two rings are fused together and each ring is independently selected from carbocycle, heterocycle, aryl, and heteroaryl. Non-limiting examples of bicycle groups include:

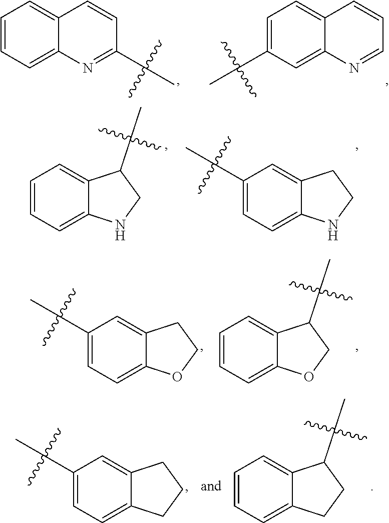

When the term “bicycle” is used in the context of a bivalent residue such as R²⁰, R²¹, R²², R²³, or R²⁴, the attachment points can be on separate rings or on the same ring. In certain embodiments both attachment points are on the same ring. In certain embodiments both attachment points are on different rings. Non-limiting examples of bivalent bicycle groups include:

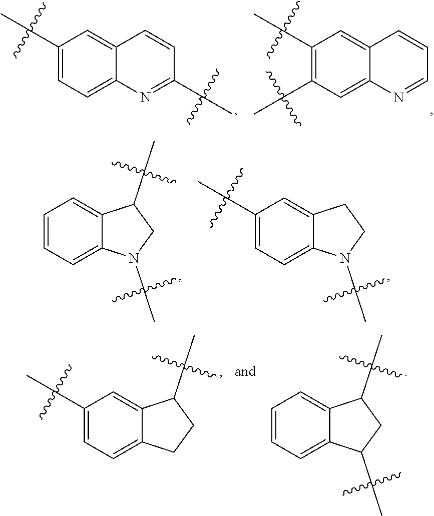 [SMILES: *c1ccc2nc(*)ccc2c1 ; *c1cc2cccnc2cc1* ; *C1CN(*)c2ccccc21 ; *c1ccc2c(c1)CCN2* ; *c1ccc2c(c1)C(*)CC2 ; *C1CC(*)c2ccccc21]

“Aliphatic” refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In certain embodiments, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl. Unsaturated aliphatic groups can be in a cis or trans configuration. In certain embodiments, the aliphatic group contains from 1 to about 12 carbon atoms, more generally from 1 to about 6 carbon atoms or from 1 to about 4 carbon atoms. In certain embodiments, the aliphatic group contains from 1 to about 8 carbon atoms. In certain embodiments, the aliphatic group is C₁-C₂, C₁-C₃, C₁-C₄, C₁-C₅ or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In certain embodiments, the aliphatic group is substituted with one or more functional groups that results in the formation of a stable moiety.

The term “heteroaliphatic” refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In certain embodiments, the only heteroatom is nitrogen. In certain embodiments, the only heteroatom is oxygen. In certain embodiments, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In certain embodiments, “heteroaliphatic” is used to indicate a heteroaliphatic group (cyclic, acyclic, substituted, unsubstituted, branched or unbranched) having 1-20 carbon atoms. In certain embodiments, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, and the like. A “dosage form” can also include an implant, for example an optical implant.

As used herein “endogenous” refers to any material from or produced inside an organism, cell, tissue or system.

As used herein, the term “exogenous” refers to any material introduced from or produced outside an organism, cell, tissue or system.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Parenteral” administration of a compound includes, e.g., subcutaneous (s.c.), intravenous (i.v.), intramuscular (i.m.), or intrasternal injection, or infusion techniques.

As used herein, “pharmaceutical compositions” is a composition comprising at least one active agent such as a selected active compound as described herein, and at least one other substance, such as a carrier. “Pharmaceutical combinations” are combinations of at least two active agents which may be combined in a single dosage form or provided together in separate dosage forms with instructions that the active agents are to be used together to treat any disorder described herein.

As used herein, a “pharmaceutically acceptable salt” is a derivative of the disclosed compound in which the parent compound is modified by making inorganic and organic, acid or base addition salts thereof with a biologically acceptable lack of toxicity. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” means a diluent, excipient, or vehicle that an active agent is used or delivered in.

A “pharmaceutically acceptable excipient” means an excipient that is useful in preparing a pharmaceutical composition/combination that is generally safe, and neither biologically nor otherwise inappropriate for administration to a host, typically a human. In certain embodiments, an excipient is used that is acceptable for veterinary use.

A “patient” or “host” or “subject” is a human or non-human animal in need of treatment, of any of the disorders as specifically described herein. Typically, the host is a human. A “host” may alternatively refer to for example, a mammal, primate (e.g., human), cow, sheep, goat, horse, dog, cat, rabbit, rat, mice, fish, bird and the like.

A “therapeutically effective amount” of a pharmaceutical composition/combination of this invention means an amount effective, when administered to a host, to provide a therapeutic benefit such as an amelioration of symptoms or reduction or diminution of the disease itself.

In certain embodiments a “prodrug” is a version of the parent molecule that is metabolized or chemically converted to the parent molecule in vivo, for example in a mammal or a human. Non-limiting examples of prodrugs include esters, amides, for example off a primary or secondary amine, carbonates, carbamates, phosphates, ketals, imines, oxazolidines, and thiazolidines. A prodrug can be designed to release the parent molecule upon a change in pH (for example in the stomach or the intestine) or upon action of an enzyme (for example an esterase or amidase).

In certain embodiments “stable” means the less than 10%, 5%, 3%, or 1% of the compound degrades under ambient conditions with a shelf life of at least 3, 4, 5, or 6-months. In certain embodiments a compound stored at ambient conditions is stored at about room temperature and exposed to air and a relative humidity of less than about 40%, 50%, 60%, or 70%. In certain embodiments a compound stored at ambient conditions is stored at about room temperature under inert gas (such as argon or nitrogen). Typically, moieties described herein do not have more than one or two heteroatoms bound to each other directly unless the moiety is heteroaromatic.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### II. Compounds of the Present Invention

### Embodiments of “Alkyl”

In certain embodiments “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

In certain embodiments “alkyl” has seven carbons.

In certain embodiments “alkyl” has eight carbons.

In certain embodiments “alkyl” has nine carbons.

In certain embodiments “alkyl” has ten carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

### Embodiments of “Haloalkyl”

In certain embodiments “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has two carbons and one halogen.

In certain embodiments “haloalkyl” has two carbons and two halogens.

In certain embodiments “haloalkyl” has two carbons and three halogens.

In certain embodiments “haloalkyl” has two carbons and four halogens.

In certain embodiments “haloalkyl” has two carbons and five halogens.

In certain embodiments “haloalkyl” has three carbons.

In certain embodiments “haloalkyl” has three carbons and one halogen.

In certain embodiments “haloalkyl” has three carbons and two halogens.

In certain embodiments “haloalkyl” has three carbons and three halogens.

In certain embodiments “haloalkyl” has three carbons and four halogens.

In certain embodiments “haloalkyl” has three carbons and five halogens.

In certain embodiments “haloalkyl” has three carbons and six halogens.

In certain embodiments “haloalkyl” has three carbons and seven halogens.

In certain embodiments “haloalkyl” has four carbons.

In certain embodiments “haloalkyl” has five carbons.

In certain embodiments “haloalkyl” has six carbons.

Non-limiting examples of “haloalkyl” include:

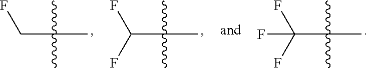

Additional non-limiting examples of “haloalkyl” include:

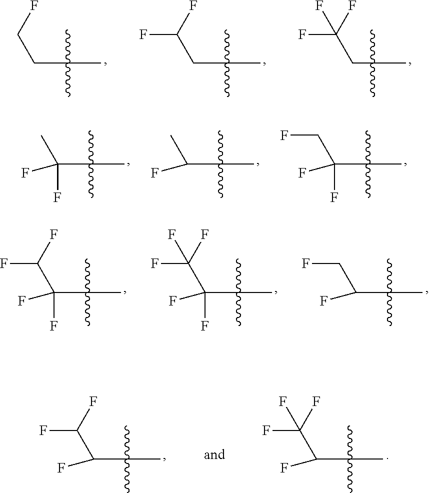

Additional non-limiting examples of “haloalkyl” include:

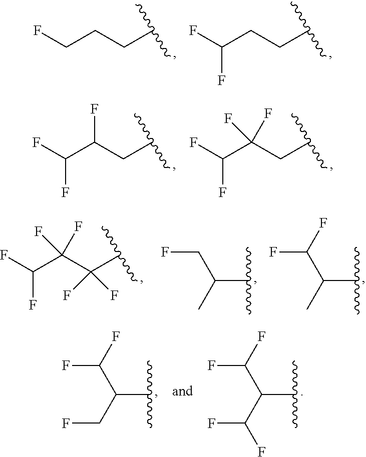

Additional non-limiting examples of “haloalkyl” include:

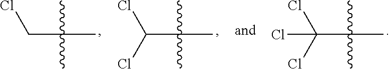

Additional non-limiting examples of “haloalkyl” include:

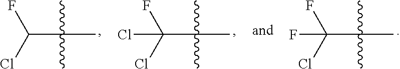

### Embodiments of “Aryl”

In certain embodiments “aryl” is a 6 carbon aromatic group (phenyl)

In certain embodiments “aryl” is a 10 carbon aromatic group (napthyl)

In certain embodiments “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example,

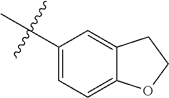 [SMILES: *c1ccc2c(c1)CCO2]

is an “aryl” group.

However,

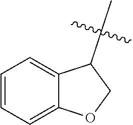 [SMILES: *C1COc2ccccc21]

is a “heterocycle” group.

In certain embodiments “aryl” is a 6 carbon aromatic group fused to a cycloalkyl wherein the point of attachment is the aryl ring. Non-limiting examples of “aryl” include dihydro-indene and tetrahydronaphthalene wherein the point of attachment for each group is on the aromatic ring.

For example,

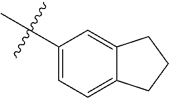 [SMILES: *c1ccc2c(c1)CCC2]

is an “aryl” group.

However,

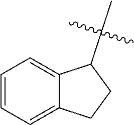 [SMILES: *C1CCc2ccccc21]

is a “cycloalkyl” group.

### Embodiments of “Heteroaryl”

In certain embodiments “heteroaryl” is a 5 membered aromatic group containing 1, 2, 3, or 4 nitrogen atoms.

Non-limiting examples of 5 membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5 membered “heteroaryl” groups include:

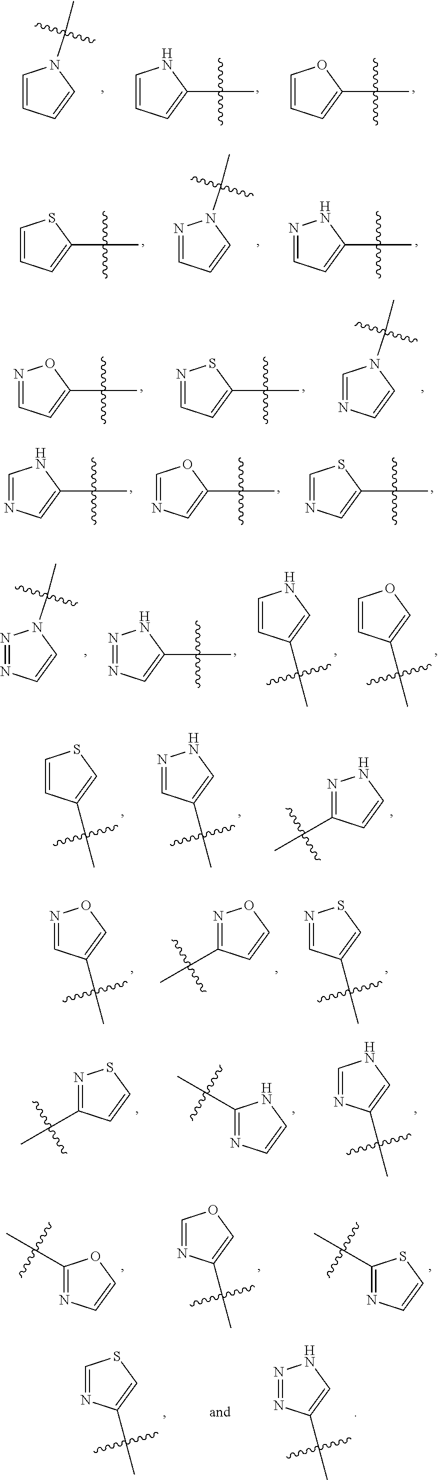

In certain embodiments “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

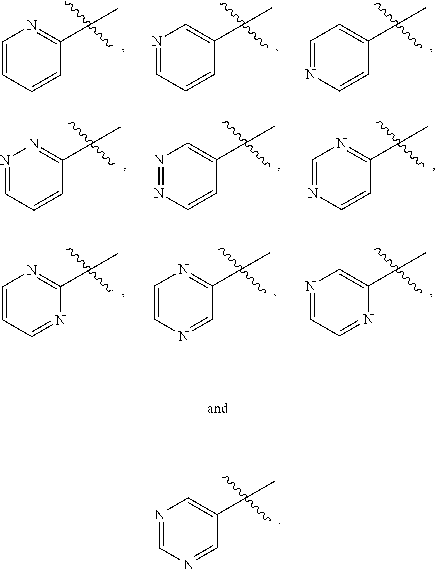

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

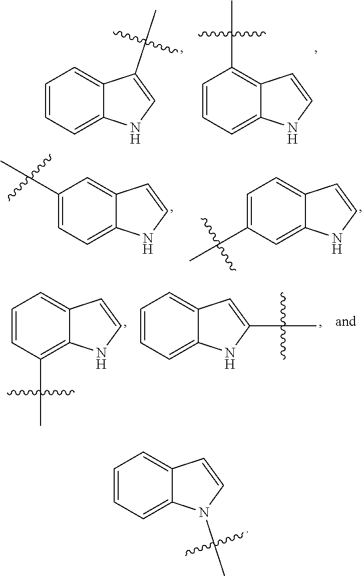

Additional non-limiting examples of “heteroaryl” groups that are bicyclic include:

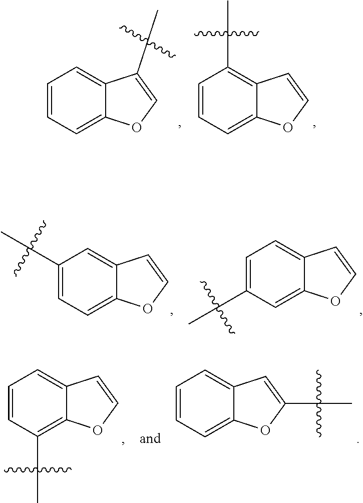

Additional non-limiting examples of “heteroaryl” groups that are bicyclic include:

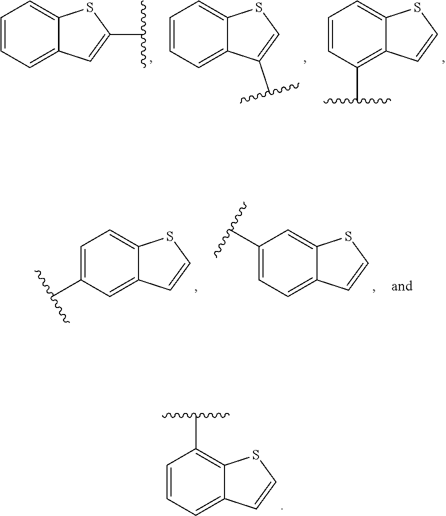

Additional non-limiting examples of “heteroaryl” groups that are bicyclic include:

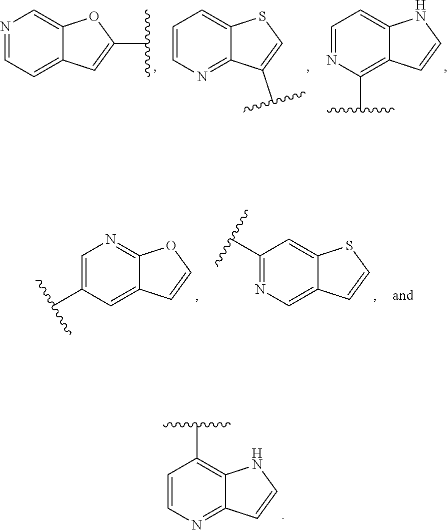

Additional non-limiting examples of “heteroaryl” groups that are bicyclic include:

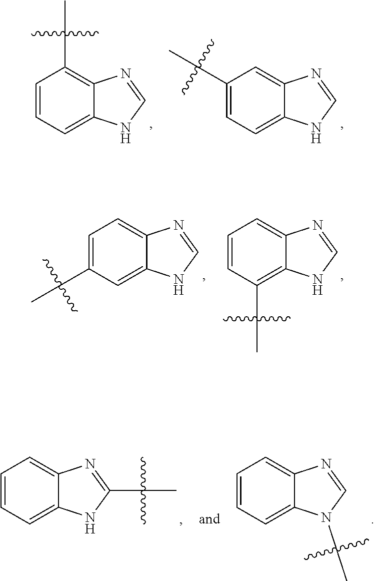

In certain embodiments “heteroaryl” is a 10 membered bicyclic aromatic group containing 1 or 2 nitrogens.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

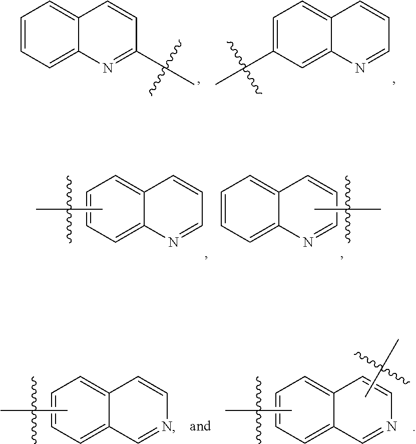

Additional non-limiting examples of “heteroaryl” groups that are bicyclic include:

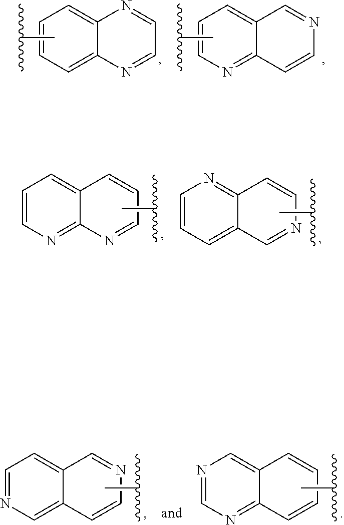

### Embodiments of “Cycloalkyl”

In certain embodiments “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In certain embodiments “cycloalkyl” has three carbons.

In certain embodiments “cycloalkyl” has four carbons.

In certain embodiments “cycloalkyl” has five carbons.

In certain embodiments “cycloalkyl” has six carbons.

In certain embodiments “cycloalkyl” has seven carbons.

In certain embodiments “cycloalkyl” has eight carbons.

In certain embodiments “cycloalkyl” has nine carbons.

In certain embodiments “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

Additional non-limiting examples of “cycloalkyl” include dihydro-indene and tetrahydronaphthalene wherein the point of attachment for each group is on the cycloalkyl ring.

For example

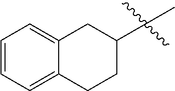 [SMILES: *C1CCc2ccccc2C1]

is an “cycloalkyl” group.

However,

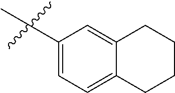 [SMILES: *c1ccc2c(c1)CCCC2]

is an aryl group.

### Embodiments of “Heterocycle”

In certain embodiments “heterocycle” refers to a cyclic ring with one nitrogen and 3, 4, 5, 6, 7, or 8 carbon atoms.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

For example,

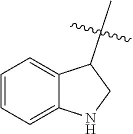 [SMILES: *C1CNc2ccccc21]

is a “heterocycle” group.

However,

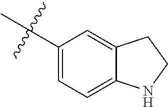 [SMILES: *c1ccc2c(c1)CCN2]

is an “aryl” group.

Non-limiting examples of “heterocycle” also include:

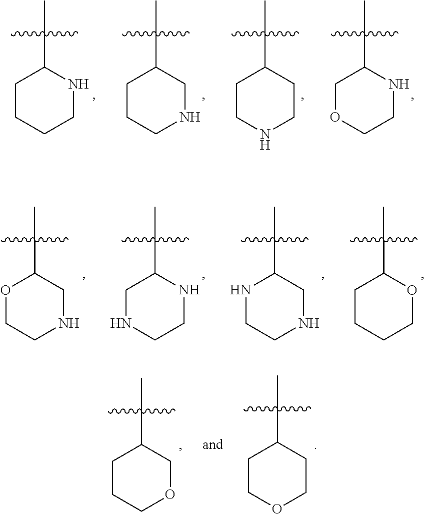

Additional non-limiting examples of “heterocycle” include:

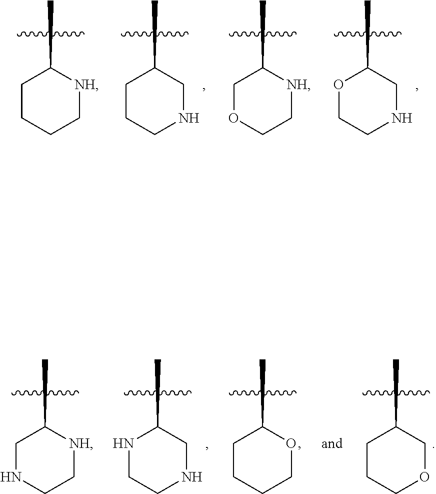

Additional non-limiting examples of “heterocycle” include:

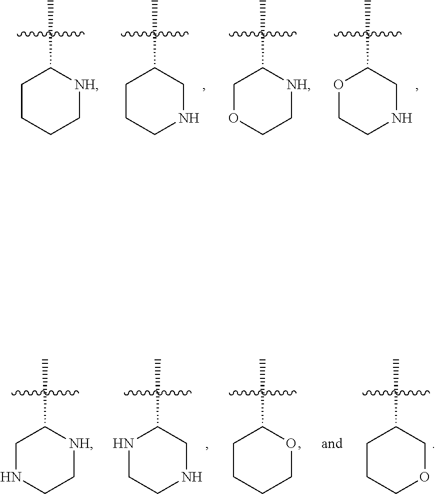

Non-limiting examples of “heterocycle” also include:

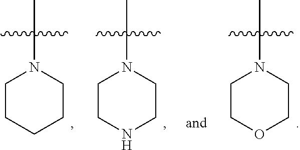

Non-limiting examples of “heterocycle” also include:

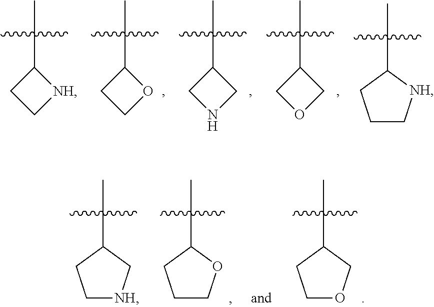

Additional non-limiting examples of “heterocycle” include:

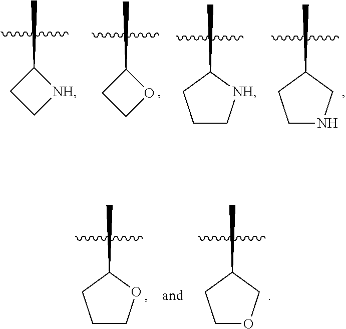

Additional non-limiting examples of “heterocycle” include:

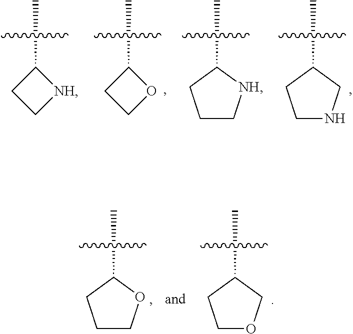

### Optional Substituents

In certain embodiments a moiety described herein that can be substituted with 1, 2, 3, or 4 substituents is substituted with one substituent.

In certain embodiments a moiety described herein that can be substituted with 1, 2, 3, or 4 substituents is substituted with two substituents.

In certain embodiments a moiety described herein that can be substituted with 1, 2, 3, or 4 substituents is substituted with three substituents.

In certain embodiments a moiety described herein that can be substituted with 1, 2, 3, or 4 substituents is substituted with four substituents.

Non-Limiting Embodiments of R¹ and/or R²

In certain embodiments each R¹ and/or R² are independently selected from alkyl, halogen, haloalkyl, —OR¹⁰, —SR¹⁰, —S(O)R¹², —SO₂R¹², —NR¹⁰R¹¹, cyano, and nitro.

In certain embodiments each R¹ and/or R² are independently selected from hydrogen, alkyl, halogen, and haloalkyl.

In certain embodiments each R¹ and/or R² are independently selected from halogen, —OR¹⁰, —SR¹⁰, —S(O)R¹², —SO₂R¹², μ-NR¹⁰R¹¹, cyano, and nitro.

In certain embodiments each R¹ and/or R² are independently selected from halogen, —S(O)R¹², —SO₂R¹², cyano, and nitro.

In certain embodiments each R¹ and/or R² are independently selected from alkyl, haloalkyl, —OR¹⁰, and —SR¹⁰.

In certain embodiments each R¹ and/or R² are independently selected from alkyl, haloalkyl, and cyano.

In certain embodiments each R¹ and/or R² is hydrogen.

In certain embodiments each R¹ and/or R² is alkyl.

In certain embodiments each R¹ and/or R² is halogen.

In certain embodiments each R¹ and/or R² is haloalkyl.

In certain embodiments each R¹ and/or R² is —OR¹⁰.

In certain embodiments each R¹ and/or R² is —SR¹⁰.

In certain embodiments each R¹ and/or R² is —S(O)R¹².

In certain embodiments each R¹ and/or R² is —SO₂R¹².

In certain embodiments each R¹ and/or R² is —NR¹⁰R¹¹.

In certain embodiments each R¹ and/or R² is cyano.

In certain embodiments each R¹ and/or R² is nitro.

In certain embodiments each R¹ and/or R² is heteroaryl.

In certain embodiments each R¹ and/or R² is aryl.

In certain embodiments each R¹ and/or R² is heterocyclic.

In certain embodiments there is only one R¹ substituent on Cycle-A or Cycle-C.

In certain embodiments there are only two R¹ substituents on Cycle-A or Cycle-C.

In certain embodiments there are three R¹ substituents on Cycle-A or Cycle-C.

In certain embodiments there is only one R¹ substituent on Cycle-B.

In certain embodiments there are only two R¹ substituents on Cycle-B.

In certain embodiments there are three R¹ substituents on Cycle-B.

In certain embodiments there is only one R² substituent on Cycle-D.

In certain embodiments there are only two R² substituents on Cycle-D.

In certain embodiments there are three R² substituents on Cycle-D.

In certain embodiments one R¹ substituent is halogen.

In certain embodiments two R¹ substituents are halogen.

In certain embodiments three R¹ substituents are halogen.

In certain embodiments one R² substituent is halogen.

In certain embodiments two R² substituents are halogen.

In certain embodiments three R² substituents are halogen.

In certain embodiments one R¹ substituent is haloalkyl.

In certain embodiments two R¹ substituents are haloalkyl.

In certain embodiments three R¹ substituents are haloalkyl.

In certain embodiments one R² substituent is haloalkyl.

In certain embodiments two R² substituents are haloalkyl.

In certain embodiments three R² substituents are haloalkyl.

In certain embodiments one R¹ substituent is alkyl.

In certain embodiments two R¹ substituents are alkyl.

In certain embodiments three R¹ substituents are alkyl.

In certain embodiments one R² substituent is alkyl.

In certain embodiments two R² substituents are alkyl.

In certain embodiments three R² substituents are alkyl.

In certain embodiments two R¹ groups are combined to form a fused phenyl ring.

In certain embodiments two R¹ groups are combined to form a fused 5-membered heteroaryl ring.

In certain embodiments two R¹ groups are combined to form a fused 6-membered heteroaryl ring.

In certain embodiments an R¹ group is combined with an R² group to form a fused 6-membered heterocycle.

In certain embodiments an R¹ group is combined with an R² group to form a fused 5-membered heterocycle.

In certain embodiments two R² groups are combined to form a fused phenyl ring.

In certain embodiments two R¹ groups are combined to form a fused phenyl ring.

In certain embodiments two R² groups are combined to form a fused 5-membered heteroaryl ring.

In certain embodiments two R² groups are combined to form a fused 6-membered heteroaryl ring.

### Non-Limiting Embodiments of R³

In certain embodiments R³ is selected from hydrogen and halogen.

In certain embodiments R³ is selected from alkyl and haloalkyl.

In certain embodiments R³ is hydrogen.

In certain embodiments R³ is halogen.

In certain embodiments R³ is alkyl.

In certain embodiments R³ is haloalkyl.

In certain embodiments R³ is fluoro.

In certain embodiments R³ is chloro.

In certain embodiments R³ is bromo.

In certain embodiments R³ is iodo.

In certain embodiments R³ is methyl.

In certain embodiments R³ is ethyl.

In certain embodiments R³ is trifluoromethyl.

In certain embodiments R³ is pentafluoroethyl.

In certain embodiments R³ is difluoromethyl.

In certain embodiments R³ is fluoromethyl.

In certain embodiments R³ is combined with an R⁴ group to form a 1 carbon attachment.

In certain embodiments R³ is combined with an R⁴ group to form a 2 carbon attachment.

In certain embodiments R³ is combined with an R⁴ group to form a 3 carbon attachment.

In certain embodiments R³ is combined with an R⁴ group to form a 4 carbon attachment.

In certain embodiments R³ is combined with an R⁴ group to form a double bond.

In certain embodiments R³ is combined with an R⁴ group to form a 1 carbon attachment.

In certain embodiments R³ is combined with an R⁴ group to form a 2 carbon attachment.

In certain embodiments R³ is combined with an R⁴ group to form a 3 carbon attachment.

In certain embodiments R³ is combined with an R⁴ group to form a 4 carbon attachment.

Non-Limiting Embodiments of R⁶ and R⁷

In certain embodiments R⁶ and R⁷ are independently selected from hydrogen, alkyl, halogen, and haloalkyl.

In certain embodiments R⁶ and R⁷ are independently selected from —OR¹⁰, —SR¹⁰, —S(O)R¹², —SO₂R¹², and —NR¹⁰R¹¹.

In certain embodiments R⁶ and R⁷ are independently selected from alkyl, —OR¹⁰, —SR¹⁰, and —NR¹⁰R¹¹.

In certain embodiments R⁶ is combined with an R³group to form a 1 carbon attachment.

In certain embodiments R⁶ is combined with an R³ group to form a 2 carbon attachment.

In certain embodiments R⁶ is combined with an R³ group to form a 3 carbon attachment.

In certain embodiments R⁶ is combined with an R³ group to form a 4 carbon attachment.

Non-Limiting Embodiments of R¹⁰ and R¹¹

In certain embodiments, R¹⁰ is hydrogen.

In certain embodiments, R¹⁰ is alkyl.

In certain embodiments, R¹⁰ is haloalkyl.

In certain embodiments, R¹⁰ is heterocycle.

In certain embodiments, R¹⁰ is aryl.

In certain embodiments, R¹⁰ is heteroaryl.

In certain embodiments, R¹⁰ is —C(O)R¹².

In certain embodiments, R¹⁰ is —S(O)R¹².

In certain embodiments, R¹⁰ is —SO₂R¹².

In certain embodiments, R¹¹ is hydrogen.

In certain embodiments, R¹¹ is alkyl.

In certain embodiments, R¹¹ is haloalkyl.

In certain embodiments, R¹¹ is heterocycle.

In certain embodiments, R¹¹ is aryl.

In certain embodiments, R¹¹ is heteroaryl.

In certain embodiments, R¹¹ is —C(O)R¹².

In certain embodiments, R¹¹ is —S(O)R¹².

In certain embodiments, R¹¹ is —SO₂R¹².

### Non-Limiting Embodiments of R²:

In certain embodiments, R¹² is hydrogen.

In certain embodiments, R¹² is alkyl.

In certain embodiments, R¹² is haloalkyl.

In certain embodiments, R¹² is heterocycle.

In certain embodiments, R¹² is aryl.

In certain embodiments, R¹² is heteroaryl.

In certain embodiments, R¹² is —NR¹³R¹⁴.

In certain embodiments, R¹² is OR¹³.

### Non-Limiting Embodiments of R¹³:

In certain embodiments, R¹³ is hydrogen.

In certain embodiments, R¹³ is alkyl.

In certain embodiments, R¹³ is haloalkyl.

In certain embodiments, R¹⁴ is hydrogen.

In certain embodiments, R¹⁴ is alkyl.

In certain embodiments, R¹⁴ is haloalkyl.

In certain embodiments, R¹³ is hydrogen and R¹⁴ is hydrogen.

In certain embodiments, R¹³ is hydrogen and R¹⁴ is alkyl.

In certain embodiments, R¹³ is hydrogen and R¹⁴ is haloalkyl.

In certain embodiments, R¹³ is alkyl and R¹⁴ is hydrogen

In certain embodiments, R¹³ is alkyl and R¹⁴ is alkyl.

In certain embodiments, R¹³ is alkyl and R¹⁴ is haloalkyl.

In certain embodiments, R¹³ is haloalkyl and R¹⁴ is hydrogen.

In certain embodiments, R¹³ is haloalkyl and R¹⁴ is alkyl.

In certain embodiments, R¹³ is haloalkyl and R¹⁴ is haloalkyl.

Non-Limiting Embodiments of X¹ and X²:

In certain embodiments, X¹ is bond.

In certain embodiments, X¹ is heterocycle.

In certain embodiments, X¹ is heteroaryl.

In certain embodiments, X¹ is aryl.

In certain embodiments, X¹ is bicycle.

In certain embodiments, X¹ is alkyl.

In certain embodiments, X¹ is aliphatic.

In certain embodiments, X¹ is heteroaliphatic.

In certain embodiments, X¹ is —C(NR²⁷)—.

In certain embodiments, X¹ is CR⁴⁰R⁴¹—.

In certain embodiments, X¹ is —C(O)—.

In certain embodiments, X¹ is —C(NR²⁷)—.

In certain embodiments, X¹ is —C(S)—.

In certain embodiments, X¹ is —S(O)—.

In certain embodiments, X¹ is —S(O)₂—.

In certain embodiments, X¹ is —S—.

In certain embodiments, X¹ is a 5-membered aromatic heterocycle with attachment points in a 1,3 orientation.

In certain embodiments, X¹ is a 5-membered aromatic heterocycle with attachment points in a 1,2 orientation.

In certain embodiments, X¹ is a 6-membered aromatic heterocycle with attachment points in a 1,2 orientation.

In certain embodiments, X¹ is a 6-membered aromatic heterocycle with attachment points in a 1,3 orientation.

In certain embodiments, X¹ is a 6-membered aromatic heterocycle with attachment points in a 1,4 orientation.

In certain embodiments, X¹ is a 6-membered aromatic heterocycle with attachment points in a 1,3 orientation.

In certain embodiments, X¹ is a 5-membered heterocycle with attachment points in a 1,2 orientation

In certain embodiments, X¹ is a 5-membered heterocycle with attachment points in a 1,3 orientation.

In certain embodiments, X¹ is a 6-membered heterocycle with attachment points in a 1,2 orientation.

In certain embodiments, X¹ is a 6-membered heterocycle with attachment points in a 1,3 orientation.

In certain embodiments, X¹ is a 6-membered heterocycle with attachment points in a 1,4 orientation.

In certain embodiments, X¹ is a bicyclic heterocycle with one heteroatom

In certain embodiments, X¹ is a bicyclic heterocycle with two heteroatoms.

In certain embodiments, X¹ is a bicyclic heterocycle with one heteroatom and one attachment is bound to Nitrogen and one is bound to carbon

In certain embodiments, X¹ is a bicyclic heterocycle with one heteroatom, and both attachment points are bound to carbon

In certain embodiments, X¹ is a bicyclic heterocycle with two heteroatoms and both points of attachment are bound to Nitrogen.

In certain embodiments, X¹ is a bicyclic heterocycle with two heteroatoms.

In certain embodiments, X¹ is a fused bicyclic alkane.

In certain embodiments, X¹ is a spiro-bicyclic alkane.

In certain embodiments, X¹ is selected from:

In certain embodiments, X² is bond.

In certain embodiments, X² is heterocycle.

In certain embodiments, X² is heteroaryl.

In certain embodiments, X² is aryl.

In certain embodiments, X² is bicycle.

In certain embodiments, X² is alkyl.

In certain embodiments, X² is aliphatic.

In certain embodiments, X² is heteroaliphatic.

In certain embodiments, X² is —C(NR²⁷)—.

In certain embodiments, X² is CR⁴⁰R⁴¹—.

In certain embodiments, X² is —C(O)—.

In certain embodiments, X² is —C(NR²⁷)—.

In certain embodiments, X² is —C(S)—.

In certain embodiments, X² is —S(O)—.

In certain embodiments, X² is —S(O)₂—.

In certain embodiments, X² is —S—.

In certain embodiments, X² is a 5-membered aromatic heterocycle with attachment points in a 1,3 orientation.

In certain embodiments, X² is a 5-membered aromatic heterocycle with attachment points in a 1,2 orientation.

In certain embodiments, X² is a 6-membered aromatic heterocycle with attachment points in a 1,2 orientation.

In certain embodiments, X² is a 6-membered aromatic heterocycle with attachment points in a 1,3 orientation.

In certain embodiments, X² is a 6-membered aromatic heterocycle with attachment points in a 1,4 orientation.

In certain embodiments, X² is a 6-membered aromatic heterocycle with attachment points in a 1,3 orientation.

In certain embodiments, X² is a 5-membered heterocycle with attachment points in a 1,2 orientation

In certain embodiments, X² is a 5-membered heterocycle with attachment points in a 1,3 orientation.

In certain embodiments, X² is a 6-membered heterocycle with attachment points in a 1,2 orientation.

In certain embodiments, X² is a 6-membered heterocycle with attachment points in a 1,3 orientation.

In certain embodiments, X² is a 6-membered heterocycle with attachment points in a 1,4 orientation.

In certain embodiments, X² is a bicyclic heterocycle with one heteroatom

In certain embodiments, X² is a bicyclic heterocycle with two heteroatoms.

In certain embodiments, X² is a bicyclic heterocycle with one heteroatom and one attachment is bound to Nitrogen and one is bound to carbon

In certain embodiments, X² is a bicyclic heterocycle with one heteroatom, and both attachment points are bound to carbon

In certain embodiments, X² is a bicyclic heterocycle with two heteroatoms and both points of attachment are bound to Nitrogen.

In certain embodiments, X² is a bicyclic heterocycle with two heteroatoms.

In certain embodiments, X² is a fused bicyclic alkane.

In certain embodiments, X² is a spiro-bicyclic alkane.

Non-Limiting Embodiments of X³:

In certain embodiments, X³ is bond.

In certain embodiments, X³ is heterocycle.

In certain embodiments, X³ is heteroaryl.

In certain embodiments, X³ is aryl.

In certain embodiments, X³ is bicycle.

In certain embodiments, X³ is alkyl.

In certain embodiments, X³ is aliphatic.

In certain embodiments, X³ is heteroaliphatic.

In certain embodiments, X³ is —C(NR²⁷)—.

In certain embodiments, X³ is CR⁴⁰R⁴¹—.

In certain embodiments, X³ is —C(O)—.

In certain embodiments, X³ is —C(NR²⁷)—.

In certain embodiments, X³ is —C(S)—.

In certain embodiments, X³ is —S(O)—.

In certain embodiments, X³ is —S(O)₂—.

In certain embodiments, X³ is —S—.

In certain embodiments, X³ is a 5-membered aromatic heterocycle with attachment points in a 1,3 orientation.

In certain embodiments, X³ is a 5-membered aromatic heterocycle with attachment points in a 1,2 orientation.

In certain embodiments, X³ is a 6-membered aromatic heterocycle with attachment points in a 1,2 orientation.

In certain embodiments, X³ is a 6-membered aromatic heterocycle with attachment points in a 1,3 orientation.

In certain embodiments, X³ is a 6-membered aromatic heterocycle with attachment points in a 1,4 orientation.

In certain embodiments, X³ is a 6-membered aromatic heterocycle with attachment points in a 1,3 orientation.

In certain embodiments, X³ is a 5-membered heterocycle with attachment points in a 1,2 orientation

In certain embodiments, X³ is a 5-membered heterocycle with attachment points in a 1,3 orientation.

In certain embodiments, X³ is a 6-membered heterocycle with attachment points in a 1,2 orientation.

In certain embodiments, X³ is a 6-membered heterocycle with attachment points in a 1,3 orientation.

In certain embodiments, X³ is a 6-membered heterocycle with attachment points in a 1,4 orientation.

In certain embodiments, X³ is a bicyclic heterocycle with one heteroatom

In certain embodiments, X³ is a bicyclic heterocycle with two heteroatoms.

In certain embodiments, X³ is a bicyclic heterocycle with one heteroatom and one attachment is bound to Nitrogen and one is bound to carbon

In certain embodiments, X³ is a bicyclic heterocycle with one heteroatom, and both attachment points are bound to carbon

In certain embodiments, X³ is a bicyclic heterocycle with two heteroatoms and both points of attachment are bound to Nitrogen.

In certain embodiments, X³ is a bicyclic heterocycle with two heteroatoms.

In certain embodiments, X³ is a fused bicyclic alkane.

In certain embodiments, X³ is a spiro-bicyclic alkane.

Non-Limiting Embodiments of R⁵, R¹⁶, and R⁷:

In certain embodiments, R¹⁵ is bond.

In certain embodiments, R¹⁵ is alkyl.

In certain embodiments, R¹⁵ is —C(O)—.

In certain embodiments, R¹⁵ is —C(O)O—.

In certain embodiments, R¹⁵ is —OC(O)—.

In certain embodiments, R¹⁵ is —SO₂—.

In certain embodiments, R¹⁵ is —S(O)—.

In certain embodiments, R¹⁵ is —C(S)—.

In certain embodiments, R¹⁵ is C(O)NR²⁷—.

In certain embodiments, R¹⁵ is —NR²⁷C(O)—.

In certain embodiments, R¹⁵ is —O—.

In certain embodiments, R¹⁵ is —S—.

In certain embodiments, R¹⁵ is —NR²⁷—.

In certain embodiments, R¹⁵ is C(R⁴⁰R⁴¹)—.

In certain embodiments, R¹⁵ is P(O)(OR²⁶)O—.

In certain embodiments, R¹⁵ is —P(O)(OR²⁶)—.

In certain embodiments, R¹⁵ is bicycle.

In certain embodiments, R¹⁵ is alkene.

In certain embodiments, R¹⁵ is alkyne.

In certain embodiments, R¹⁵ is haloalkyl.

In certain embodiments, R¹⁵ is alkoxy.

In certain embodiments, R¹⁵ is aryl

In certain embodiments, R¹⁵ is heterocycle.

In certain embodiments, R¹⁵ is heteroaliphatic.

In certain embodiments, R¹⁵ is heteroaryl.

In certain embodiments, R¹⁵ is lactic acid

In certain embodiments, R¹⁵ is glycolic acid.

In certain embodiments, R¹⁵ is arylalkyl.

In certain embodiments, R¹⁵ is heterocyclealkyl.

In certain embodiments, R¹⁵ is heteroarylalkyl.

In certain embodiments, R¹⁶ is bond.

In certain embodiments, R¹⁶ is alkyl.

In certain embodiments, R¹⁶ is —C(O)—.

In certain embodiments, R¹⁶ is —C(O)O—.

In certain embodiments, R¹⁶ is —OC(O)—.

In certain embodiments, R¹⁶ is —SO₂—.

In certain embodiments, R¹⁶ is —S(O)—.

In certain embodiments, R¹⁶ is —C(S)—.

In certain embodiments, R¹⁶ is C(O)NR²⁷—.

In certain embodiments, R¹⁶ is —NR²⁷C(O)—.

In certain embodiments, R¹⁶ is —O—.

In certain embodiments, R¹⁶ is —S—.

In certain embodiments, R¹⁶ is —NR²⁷—.

In certain embodiments, R¹⁶ is C(R⁴⁰R⁴¹)—.

In certain embodiments, R¹⁶ is P(O)(OR²⁶)O—.

In certain embodiments, R¹⁶ is —P(O)(OR²⁶)—.

In certain embodiments, R¹⁶ is bicycle.

In certain embodiments, R¹⁶ is alkene.

In certain embodiments, R¹⁶ is alkyne.

In certain embodiments, R¹⁶ is haloalkyl.

In certain embodiments, R¹⁶ is alkoxy.

In certain embodiments, R¹⁶ is aryl

In certain embodiments, R¹⁶ is heterocycle.

In certain embodiments, R¹⁶ is heteroaliphatic.

In certain embodiments, R¹⁶ is heteroaryl.

In certain embodiments, R¹⁶ is lactic acid

In certain embodiments, R¹⁶ is glycolic acid.

In certain embodiments, R¹⁶ is arylalkyl.

In certain embodiments, R¹⁶ is heterocyclealkyl.

In certain embodiments, R¹⁶ is heteroarylalkyl.

In certain embodiments, R¹⁷ is bond.

In certain embodiments, R¹⁷ is alkyl.

In certain embodiments, R¹⁷ is —C(O)—.

In certain embodiments, R¹⁷ is —C(O)O—.

In certain embodiments, R¹⁷ is —OC(O)—.

In certain embodiments, R¹⁷ is —SO₂—.

In certain embodiments, R¹⁷ is —S(O)—.

In certain embodiments, R¹⁷ is —C(S)—.

In certain embodiments, R¹⁷ is C(O)NR²⁷—.

In certain embodiments, R¹⁷ is —NR²⁷C(O)—.

In certain embodiments, R¹⁷ is —O—.

In certain embodiments, R¹⁷ is —S—.

In certain embodiments, R¹⁷ is —NR²⁷—.

In certain embodiments, R¹⁷ is C(R⁴⁰R⁴¹)—.

In certain embodiments, R¹⁷ is P(O)(OR²⁶)O—.

In certain embodiments, R¹⁷ is —P(O)(OR²⁶)—.

In certain embodiments, R¹⁷ is bicycle.

In certain embodiments, R¹⁷ is alkene.

In certain embodiments, R¹⁷ is alkyne.

In certain embodiments, R¹⁷ is haloalkyl.

In certain embodiments, R¹⁷ is alkoxy.

In certain embodiments, R¹⁷ is aryl

In certain embodiments, R¹⁷ is heterocycle.

In certain embodiments, R¹⁷ is heteroaliphatic.

In certain embodiments, R¹⁷ is heteroaryl.

In certain embodiments, R¹⁷ is lactic acid

In certain embodiments, R¹⁷ is glycolic acid.

In certain embodiments, R¹⁷ is arylalkyl.

In certain embodiments, R¹⁷ is heterocyclealkyl.

In certain embodiments, R¹⁷ is heteroarylalkyl.

Non-Limiting Embodiments of R²⁰, R^{2′}, and R²², R²³, and R²⁴:

In certain embodiments, R²⁰ is bond.

In certain embodiments, R²⁰ is alkyl.

In certain embodiments, R²⁰ is —C(O)—.

In certain embodiments, R²⁰ is —C(O)O—.

In certain embodiments, R²⁰ is —OC(O)—.

In certain embodiments, R²⁰ is —SO₂—.

In certain embodiments, R²⁰ is —S(O)—.

In certain embodiments, R²⁰ is —C(S)—.

In certain embodiments, R²⁰ is C(O)NR²⁷—.

In certain embodiments, R²⁰ is —NR²⁷C(O)—.

In certain embodiments, R²⁰ is —O—.

In certain embodiments, R²⁰ is —S—.

In certain embodiments, R²⁰ is —NR²⁷—.

In certain embodiments, R²⁰ is C(R⁴⁰R⁴¹)—.

In certain embodiments, R²⁰ is P(O)(OR²⁶)O—.

In certain embodiments, R²⁰ is —P(O)(OR²⁶)—.

In certain embodiments, R²⁰ is bicycle.

In certain embodiments, R²⁰ is alkene.

In certain embodiments, R²⁰ is alkyne.

In certain embodiments, R²⁰ is haloalkyl.

In certain embodiments, R²⁰ is alkoxy.

In certain embodiments, R²⁰ is aryl

In certain embodiments, R²⁰ is heterocycle.

In certain embodiments, R²⁰ is heteroaliphatic.

In certain embodiments, R²⁰ is heteroaryl.

In certain embodiments, R²⁰ is lactic acid

In certain embodiments, R²⁰ is glycolic acid.

In certain embodiments, R²⁰ is arylalkyl.

In certain embodiments, R²⁰ is heterocyclealkyl.

In certain embodiments, R²⁰ is heteroarylalkyl.

In certain embodiments, R²¹ is bond.

In certain embodiments, R²¹ is alkyl.

In certain embodiments, R²¹ is —C(O)—.

In certain embodiments, R²¹ is —C(O)O—.

In certain embodiments, R²¹ is —OC(O)—.

In certain embodiments, R²¹ is —SO₂—.

In certain embodiments, R²¹ is —S(O)—.

In certain embodiments, R²¹ is —C(S)—.

In certain embodiments, R²¹ is C(O)NR²⁷—.

In certain embodiments, R²¹ is —NR²⁷C(O)—.

In certain embodiments, R²¹ is —O—.

In certain embodiments, R²¹ is —S—.

In certain embodiments, R²¹ is —NR²⁷—.

In certain embodiments, R²¹ is C(R⁴⁰R⁴¹)—.

In certain embodiments, R²¹ is P(O)(OR²⁶)O—.

In certain embodiments, R²¹ is —P(O)(OR²⁶)—.

In certain embodiments, R²¹ is bicycle.

In certain embodiments, R²¹ is alkene.

In certain embodiments, R²¹ is alkyne.

In certain embodiments, R²¹ is haloalkyl.

In certain embodiments, R²¹ is alkoxy.

In certain embodiments, R²¹ is aryl

In certain embodiments, R²¹ is heterocycle.

In certain embodiments, R²¹ is heteroaliphatic.

In certain embodiments, R²¹ is heteroaryl.

In certain embodiments, R²¹ is lactic acid

In certain embodiments, R²¹ is glycolic acid.

In certain embodiments, R²¹ is arylalkyl.

In certain embodiments, R²¹ is heterocyclealkyl.

In certain embodiments, R²¹ is heteroarylalkyl.

In certain embodiments, R²² is bond.

In certain embodiments, R²² is alkyl.

In certain embodiments, R²² is —C(O)—.

In certain embodiments, R²² is —C(O)O—.

In certain embodiments, R²² is —OC(O)—.

In certain embodiments, R²² is —SO₂—.

In certain embodiments, R²² is —S(O)—.

In certain embodiments, R²² is —C(S)—.

In certain embodiments, R²² is C(O)NR²⁷—.

In certain embodiments, R²² is —NR²⁷C(O)—.

In certain embodiments, R²² is —O—.

In certain embodiments, R²² is —S—.

In certain embodiments, R²² is —NR²⁷—.

In certain embodiments, R²² is C(R⁴⁰R⁴¹)—.

In certain embodiments, R²² is P(O)(OR²⁶)O—.

In certain embodiments, R²² is —P(O)(OR²⁶)—

In certain embodiments, R²² is bicycle.

In certain embodiments, R²² is alkene.

In certain embodiments, R²² is alkyne.

In certain embodiments, R²² is haloalkyl.

In certain embodiments, R²² is alkoxy.

In certain embodiments, R²² is aryl

In certain embodiments, R²² is heterocycle.

In certain embodiments, R²² is heteroaliphatic.

In certain embodiments, R²² is heteroaryl.

In certain embodiments, R²² is lactic acid

In certain embodiments, R²² is glycolic acid.

In certain embodiments, R²² is arylalkyl.

In certain embodiments, R²² is heterocyclealkyl.

In certain embodiments, R²² is heteroarylalkyl.

In certain embodiments, R²³ is bond.

In certain embodiments, R²³ is alkyl.

In certain embodiments, R²³ is —C(O)—.

In certain embodiments, R²³ is —C(O)O—.

In certain embodiments, R²³ is —OC(O)—.

In certain embodiments, R²³ is —SO₂—.

In certain embodiments, R²³ is —S(O)—.

In certain embodiments, R²³ is —C(S)—.

In certain embodiments, R²³ is C(O)NR²⁷—.

In certain embodiments, R²³ is —NR²⁷C(O)—.

In certain embodiments, R²³ is —O—.

In certain embodiments, R²³ is —S—.

In certain embodiments, R²³ is —NR²⁷—.

In certain embodiments, R²³ is C(R⁴⁰R⁴¹)—.

In certain embodiments, R²³ is P(O)(OR²⁶)O—.

In certain embodiments, R²³ is —P(O)(OR²⁶)—.

In certain embodiments, R²³ is bicycle.

In certain embodiments, R²³ is alkene.

In certain embodiments, R²³ is alkyne.

In certain embodiments, R²³ is haloalkyl.

In certain embodiments, R²³ is alkoxy.

In certain embodiments, R²³ is aryl

In certain embodiments, R²³ is heterocycle.

In certain embodiments, R²³ is heteroaliphatic.

In certain embodiments, R²³ is heteroaryl.

In certain embodiments, R²³ is lactic acid

In certain embodiments, R²³ is glycolic acid.

In certain embodiments, R²³ is arylalkyl.

In certain embodiments, R²³ is heterocyclealkyl.

In certain embodiments, R²³ is heteroarylalkyl.

In certain embodiments, R²⁴ is bond.

In certain embodiments, R²⁴ is alkyl.

In certain embodiments, R²⁴ is —C(O)—.

In certain embodiments, R²⁴ is —C(O)O—.

In certain embodiments, R²⁴ is —OC(O)—.

In certain embodiments, R²⁴ is —SO₂—.

In certain embodiments, R²⁴ is —S(O)—.

In certain embodiments, R²⁴ is —C(S)—.

In certain embodiments, R²⁴ is C(O)NR²⁷—.

In certain embodiments, R²⁴ is —NR²⁷C(O)—.

In certain embodiments, R²⁴ is —O—.

In certain embodiments, R²⁴ is —S—.

In certain embodiments, R²⁴ is —NR²⁷—.

In certain embodiments, R²⁴ is C(R⁴⁰R⁴¹)—.

In certain embodiments, R²⁴ is P(O)(OR²⁶)O—.

In certain embodiments, R²⁴ is —P(O)(OR²⁶)—

In certain embodiments, R²⁴ is bicycle.

In certain embodiments, R²⁴ is alkene.

In certain embodiments, R²⁴ is alkyne.

In certain embodiments, R²⁴ is haloalkyl.

In certain embodiments, R²⁴ is alkoxy.

In certain embodiments, R²⁴ is aryl

In certain embodiments, R²⁴ is heterocycle.

In certain embodiments, R²⁴ is heteroaliphatic.

In certain embodiments, R²⁴ is heteroaryl.

In certain embodiments, R²⁴ is lactic acid

In certain embodiments, R²⁴ is glycolic acid.

In certain embodiments, R²⁴ is arylalkyl.

In certain embodiments, R²⁴ is heterocyclealkyl.

In certain embodiments, R²⁴ is heteroarylalkyl.

### Non-Limiting Embodiments of R²⁶:

In certain embodiments, R²⁶ is hydrogen.

In certain embodiments, R²⁶ is alkyl.

In certain embodiments, R²⁶ is arylalkyl.

In certain embodiments, R²⁶ is heteroarylalkyl.

In certain embodiments, R²⁶ is alkene.

In certain embodiments, R²⁶ is alkyne.

In certain embodiments, R²⁶ is aryl.

In certain embodiments, R²⁶ is heteroaryl.

In certain embodiments, R²⁶ is heterocycle.

In certain embodiments, R²⁶ is aliphatic.

### Non-Limiting Embodiments of R²⁷:

In certain embodiments, R²⁷ is hydrogen.

In certain embodiments, R²⁷ is alkyl.

In certain embodiments, R²⁷ is arylalkyl.

In certain embodiments, R²⁷ is heteroarylalkyl.

In certain embodiments, R²⁷ is alkene.

In certain embodiments, R²⁷ is alkyne.

In certain embodiments, R²⁷ is aryl.

In certain embodiments, R²⁷ is heteroaryl.

In certain embodiments, R²⁷ is heterocycle.

In certain embodiments, R²⁷ is aliphatic.

In certain embodiments, R²⁷ is heteroaliphatic.

In certain embodiments, R²⁷ is —C(O)(aliphatic).

In certain embodiments, R²⁷ is —C(O)(aryl).

In certain embodiments, R²⁷ is —C(O)(heteroaliphatic).

In certain embodiments, R²⁷ is —C(O)(heteroaryl).

In certain embodiments, R²⁷ is —C(O)O(aliphatic).

In certain embodiments, R²⁷ is —C(O)O(aryl).

In certain embodiments, R²⁷ is —C(O)O(heteroaliphatic).

In certain embodiments, R²⁷ is —C(O)O(heteroaryl).

### Non-Limiting Embodiments of R⁴⁰:

In certain embodiments, R⁴⁰ is hydrogen.

In certain embodiments, R⁴⁰ is R²⁷.

In certain embodiments, R⁴⁰ is alkyl.

In certain embodiments, R⁴⁰ is alkene.

In certain embodiments, R⁴⁰ is alkyne.

In certain embodiments, R⁴⁰ is fluoro.

In certain embodiments, R⁴⁰ is bromo.

In certain embodiments, R⁴⁰ is chloro.

In certain embodiments, R⁴⁰ is hydroxyl.

In certain embodiments, R⁴⁰ is alkoxy.

In certain embodiments, R⁴⁰ is azide.

In certain embodiments, R⁴⁰ is amino.

In certain embodiments, R⁴⁰ is cyano.

In certain embodiments, R⁴⁰ is —N(aliphatic, including alkyl)₂.

In certain embodiments, R⁴⁰ is —NHSO₂(aliphatic, including alkyl).

In certain embodiments, R⁴⁰ is —N(aliphatic, including alkyl)SO₂alkyl.

In certain embodiments, R⁴⁰ is —NHSO₂(aryl, heteroaryl or heterocycle.

In certain embodiments, R⁴⁰ is —N(alkyl)SO₂(aryl, heteroaryl or heterocycle).

In certain embodiments, R⁴⁰ is —NHSO₂alkenyl.

In certain embodiments, R⁴⁰ is —N(alkyl)SO₂alkenyl.

In certain embodiments, R⁴⁰ is —NHSO₂alkynyl.

In certain embodiments, R⁴⁰ is —N(alkyl)SO₂alkynyl.

In certain embodiments, R⁴⁰ is haloalkyl.

In certain embodiments, R⁴⁰ is aliphatic.

In certain embodiments, R⁴⁰ is heteroaliphatic.

In certain embodiments, R⁴⁰ is aryl.

In certain embodiments, R⁴⁰ is heteroaryl.

In certain embodiments, R⁴⁰ is heterocycle.

In certain embodiments, R⁴⁰ is oxo.

In certain embodiments, R⁴⁰ is cycloalkyl.

### Non-Limiting Examples of Compounds of Formula I, Formula II, or Formula III

In certain embodiments, the compound of the present invention is selected from:

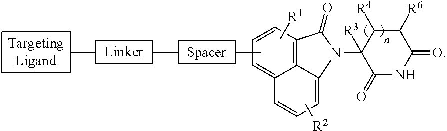

In certain embodiments, the compound of the present invention is selected from:

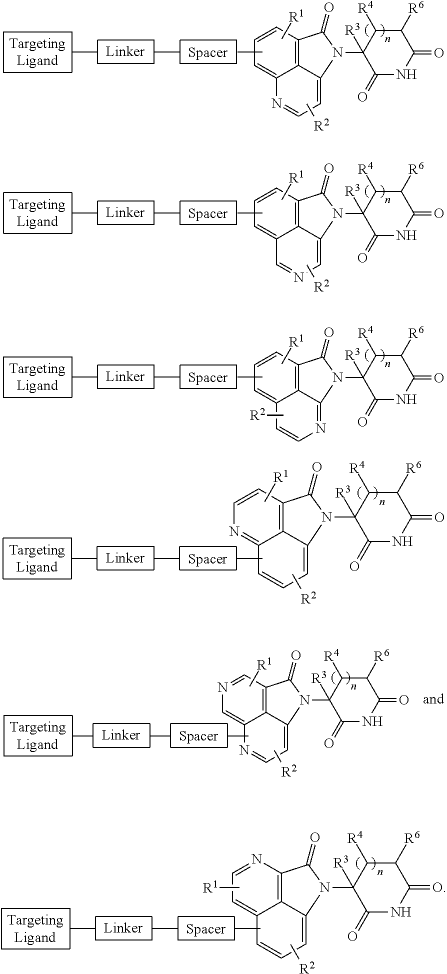

In the structures herein, a hydroxyl (for example an R¹ or R² group) is positioned on a heteroaryl ring carbon adjacent to a nitrogen, only one tautomer is shown as a shorthand method of referring individually to each separate tautomer or a mixture thereof, unless otherwise indicated herein, and each separate tautomer or mixture thereof is incorporated into the specification as if it were individually recited herein. This is demonstrated by the non-limiting examples of:

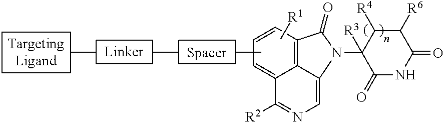

which includes both

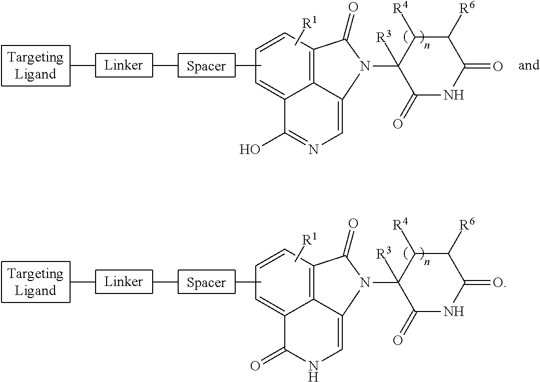

In certain embodiments, the compound of the present invention is selected from:

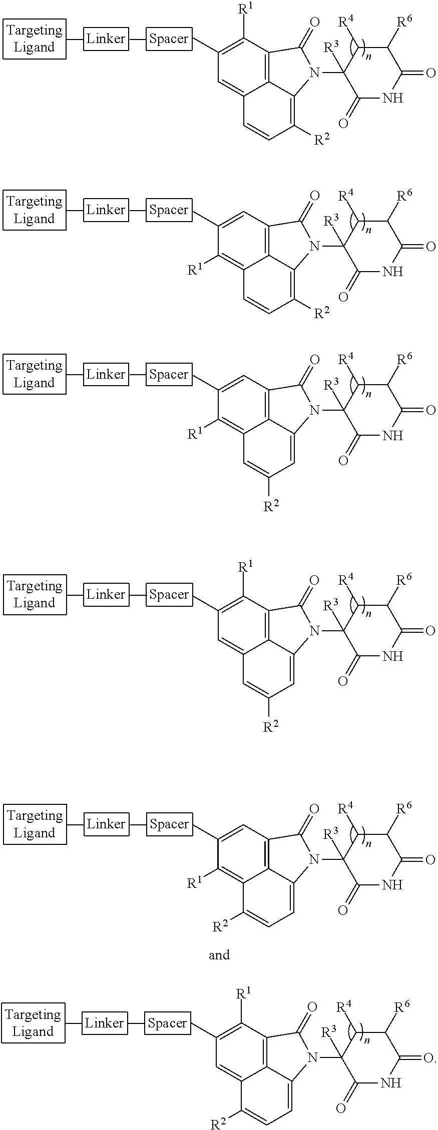

In certain embodiments, the compound of the present invention is selected from:

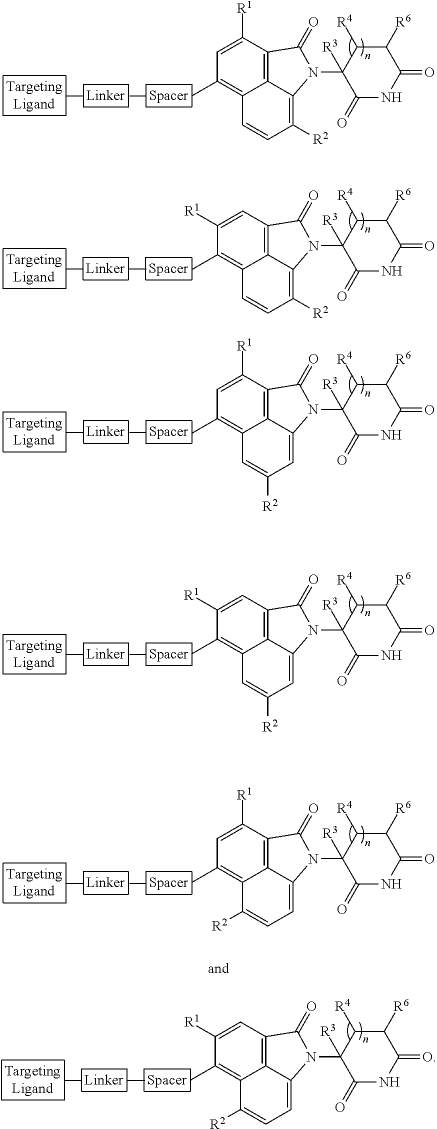

In certain embodiments, the compound of the present invention is selected from:

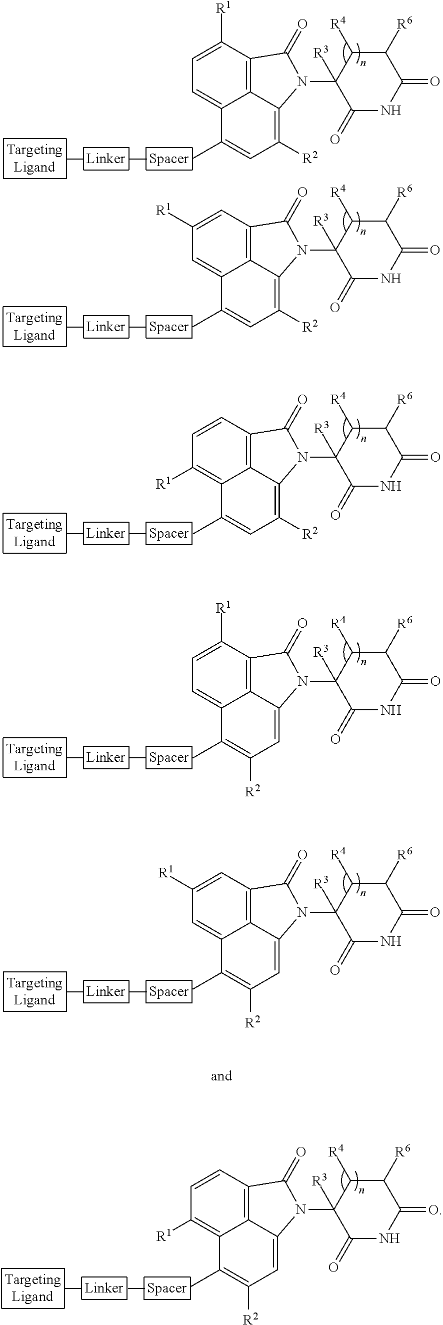

In certain embodiments, the compound of the present invention is selected from:

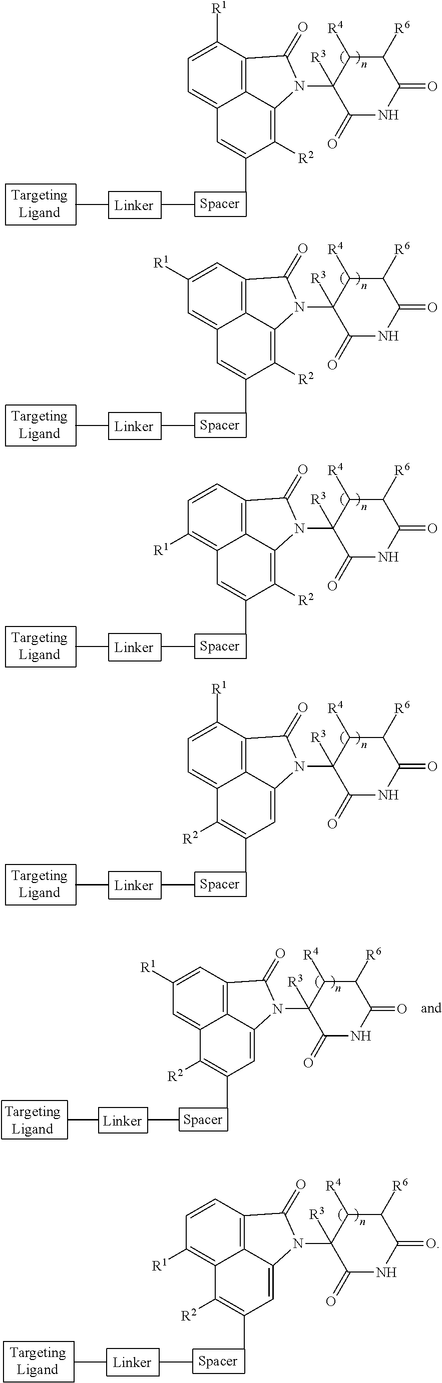

In certain embodiments, the compound of the present invention is selected from:

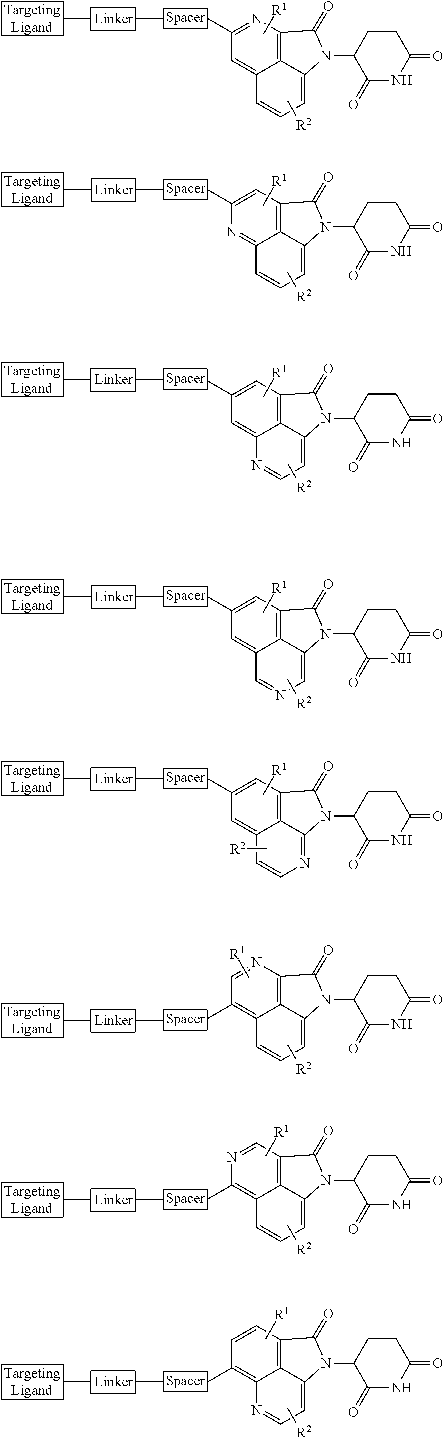

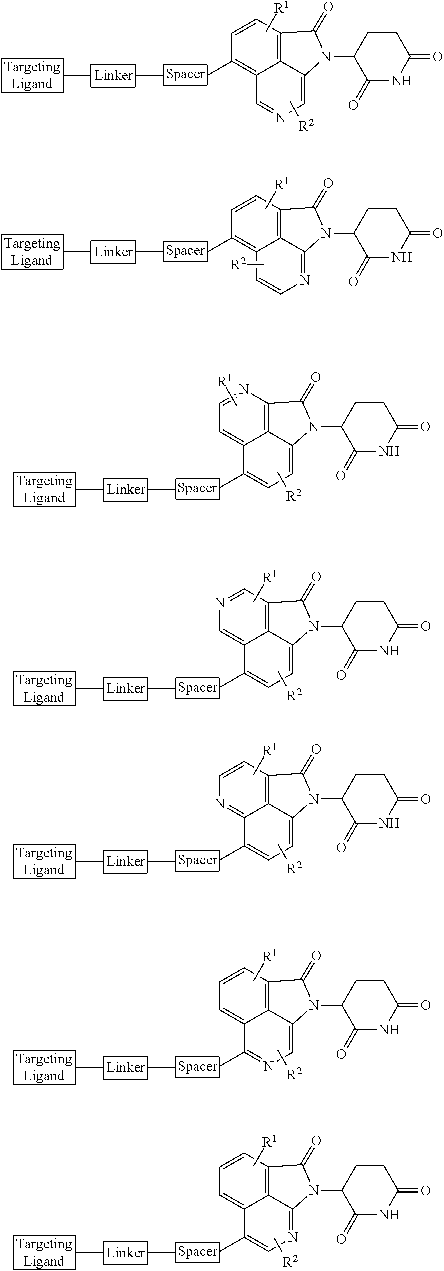

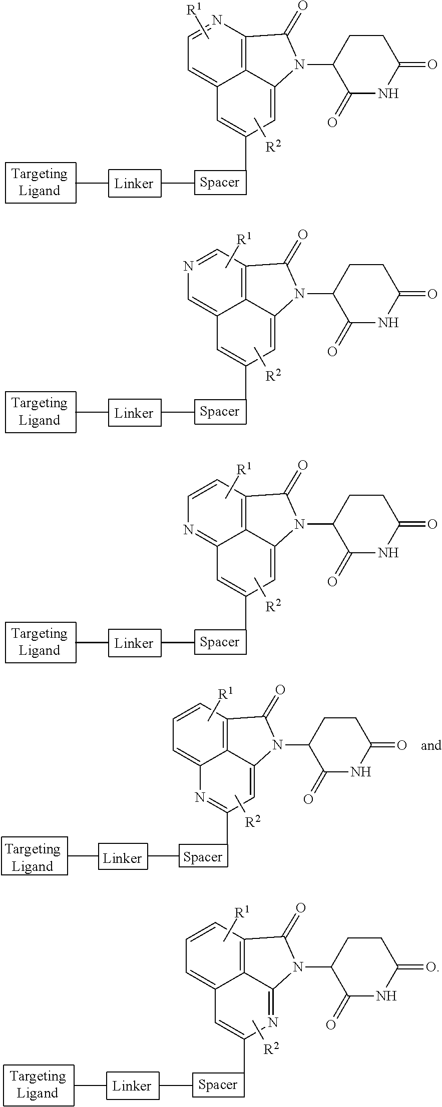

In certain embodiments, the compound of the present invention is selected from:

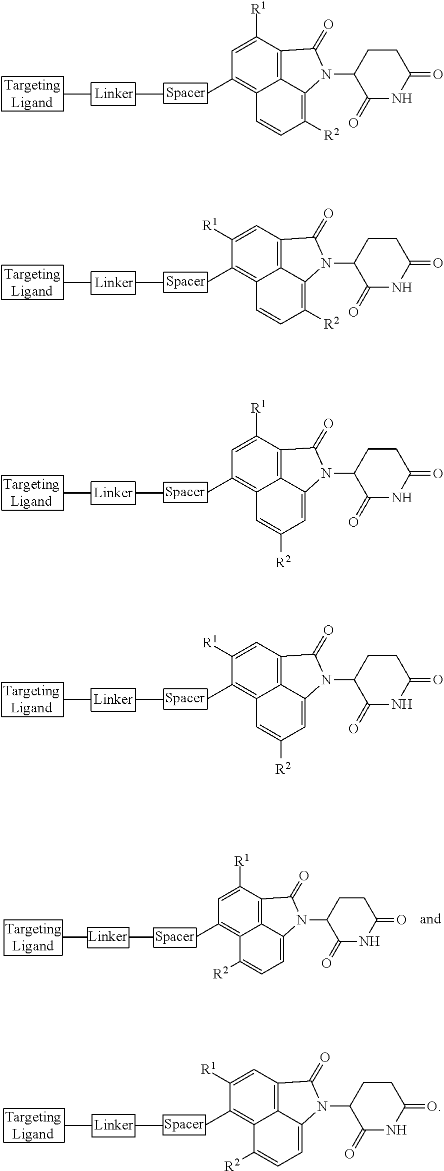

In certain embodiments, the compound of the present invention is selected from:

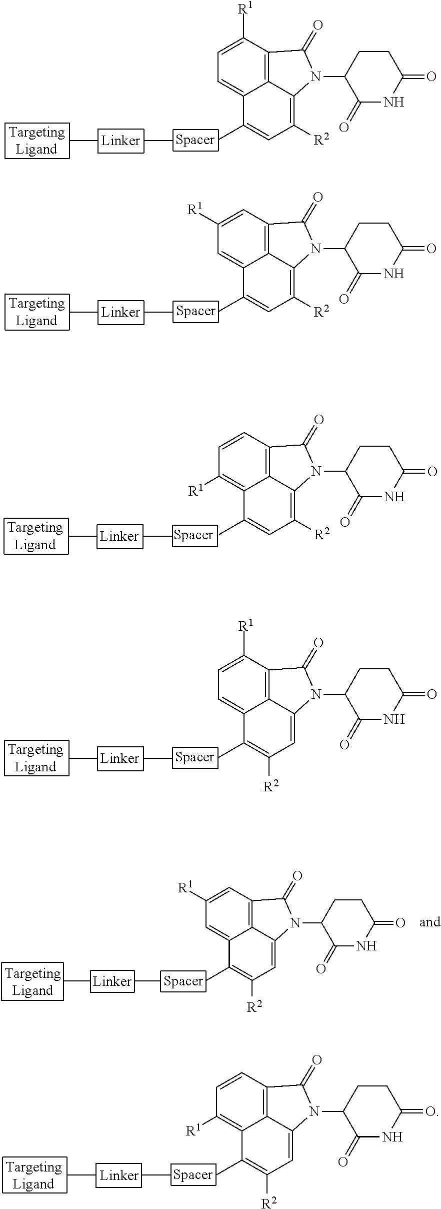

In certain embodiments, the compound of the present invention is selected from:

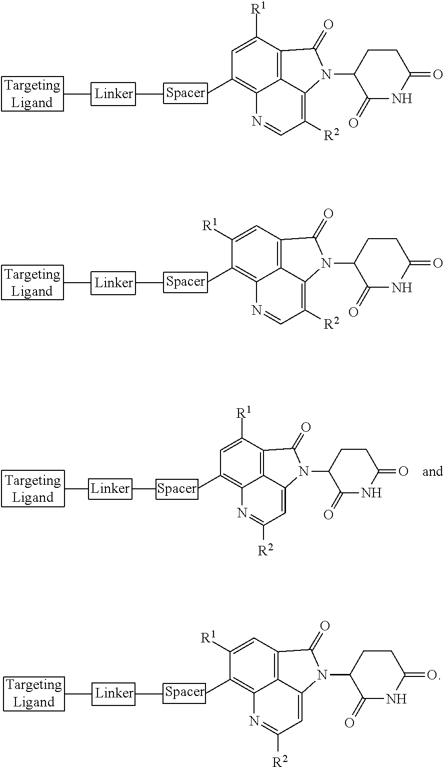

In certain embodiments, the compound of the present invention is selected from:

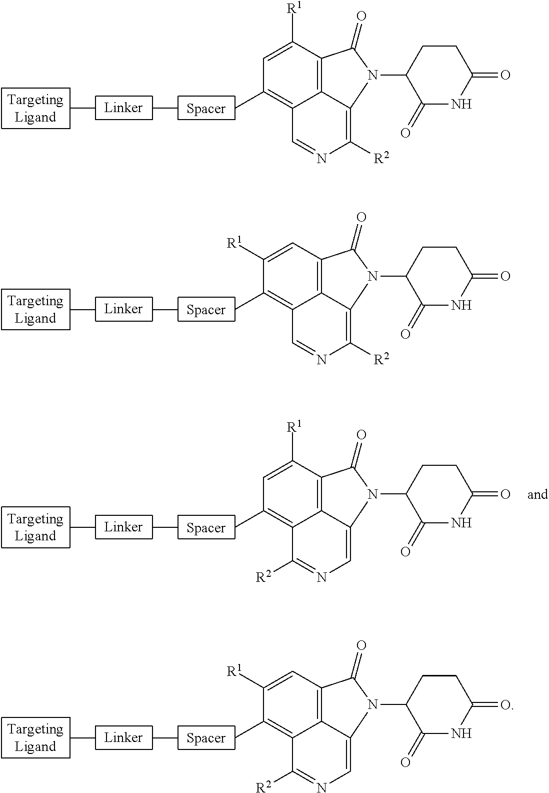

In certain embodiments, the compound of the present invention is selected from:

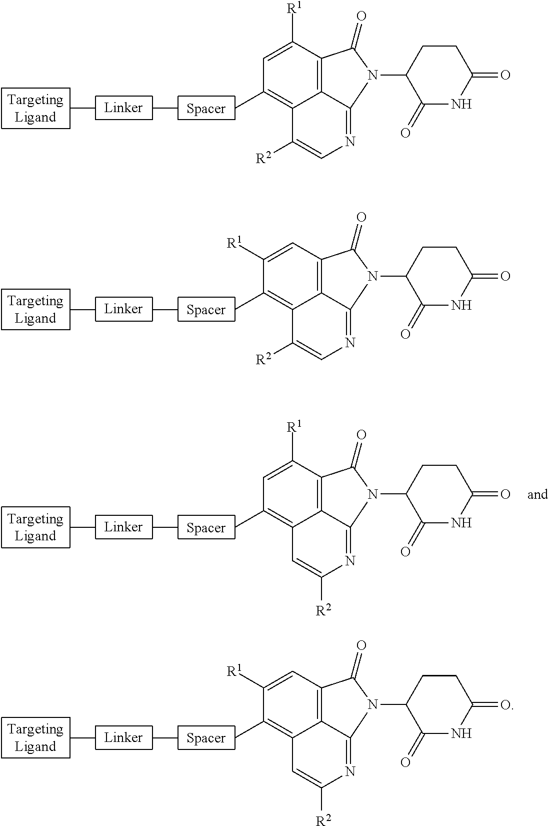

In certain embodiments, the compound of the present invention is selected from:

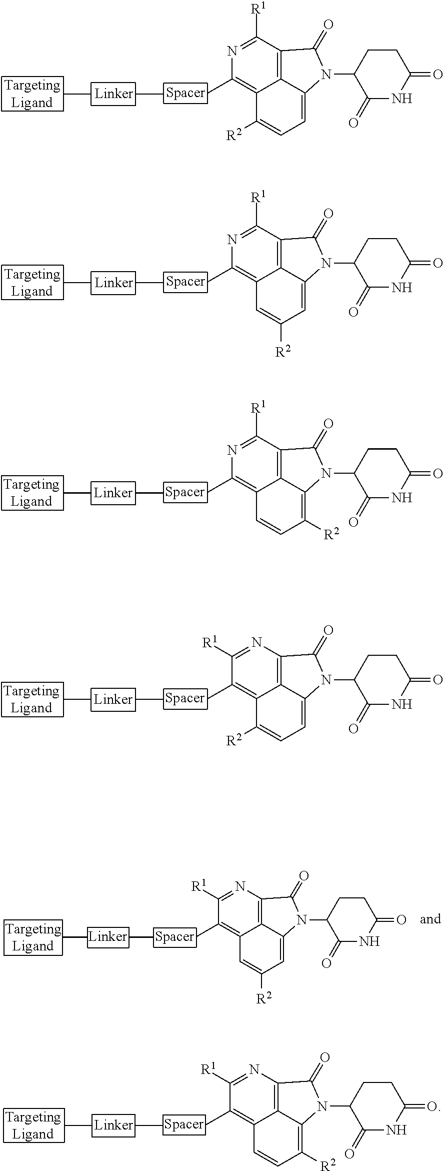

In certain embodiments, the compound of the present invention is selected from:

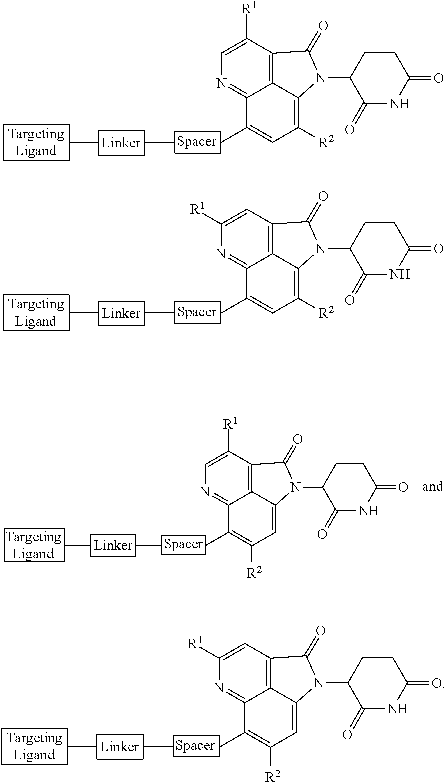

In certain embodiments, the compound of the present invention is selected from:

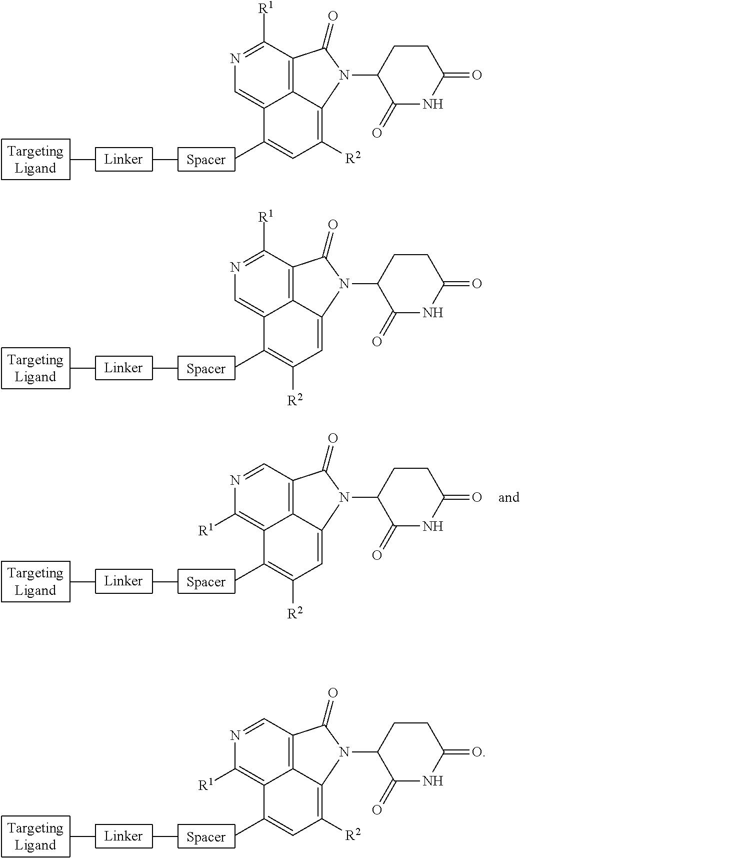

In certain embodiments, the compound of the present invention is selected from:

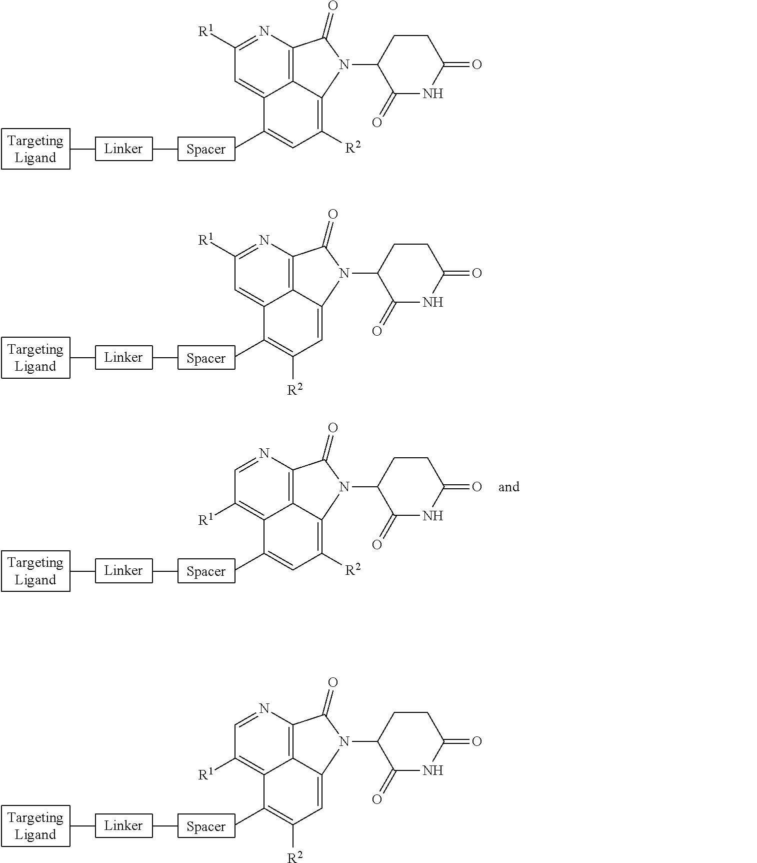

In certain embodiments, the compound of the present invention is selected from:

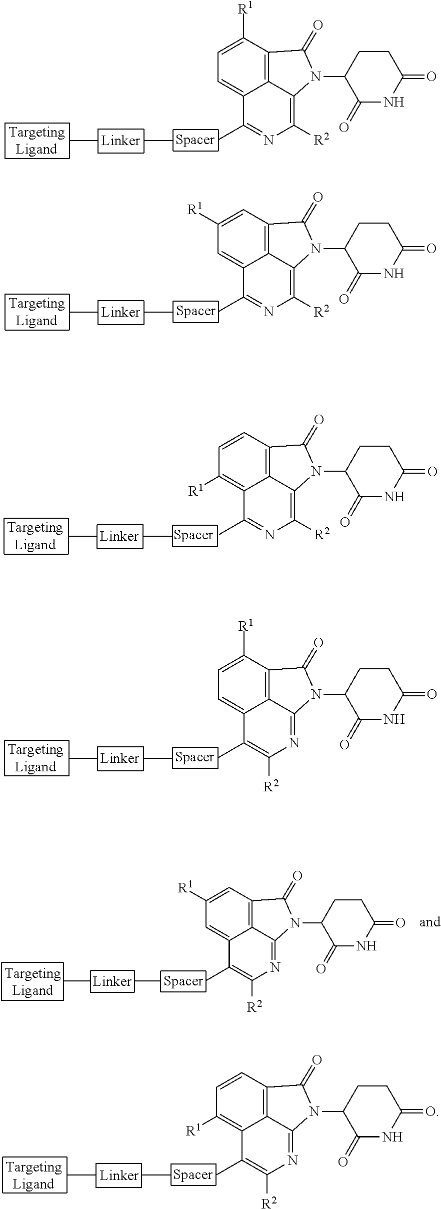

In certain embodiments the Tricyclic Cereblon Ligand is.

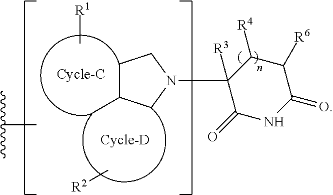

In certain embodiments spacer is bond.

### Embodiments of Cycle-A, Cycle-B, Cycle-C, and Cycle-D

In certain embodiments

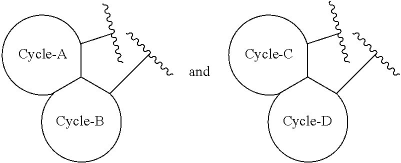

are selected from the following, wherein the dashed line indicates a potential line of attachment to the Spacer/Linker:

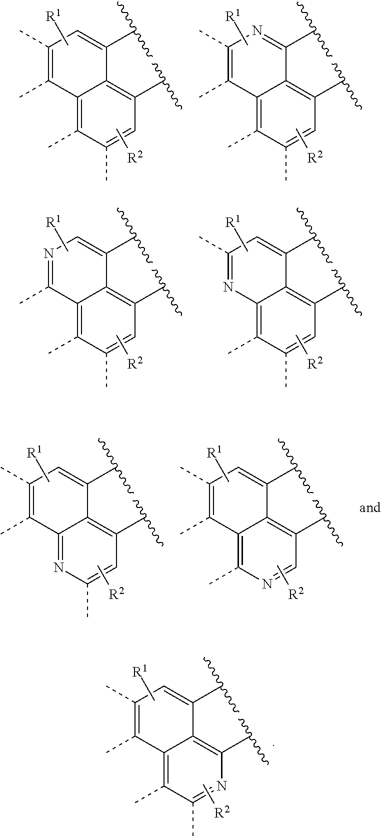

In certain embodiments

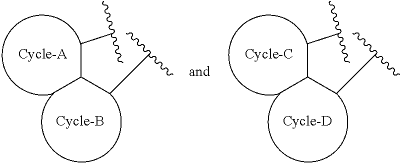

and are selected from:

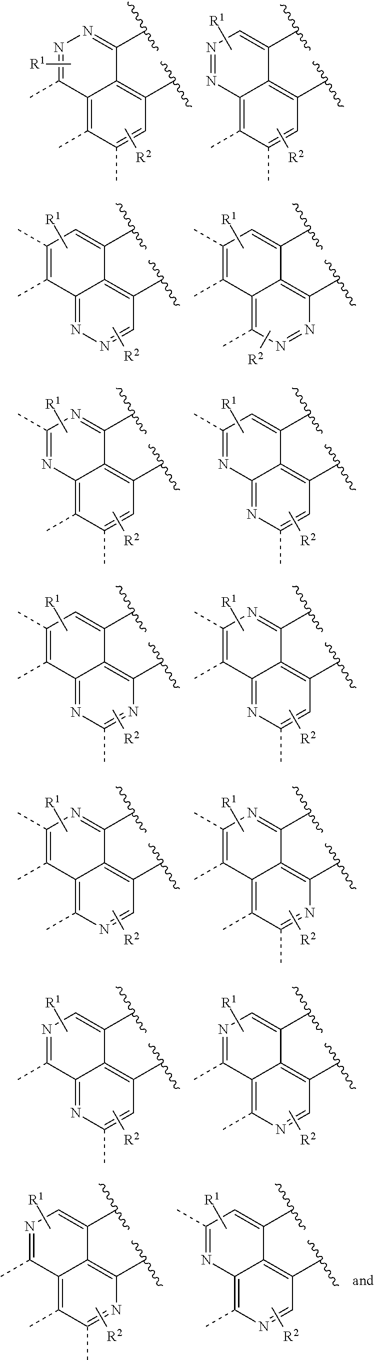

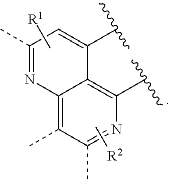

In certain embodiments

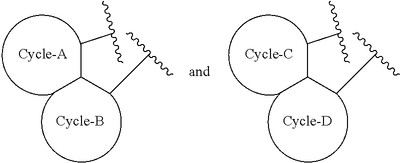

are selected from:

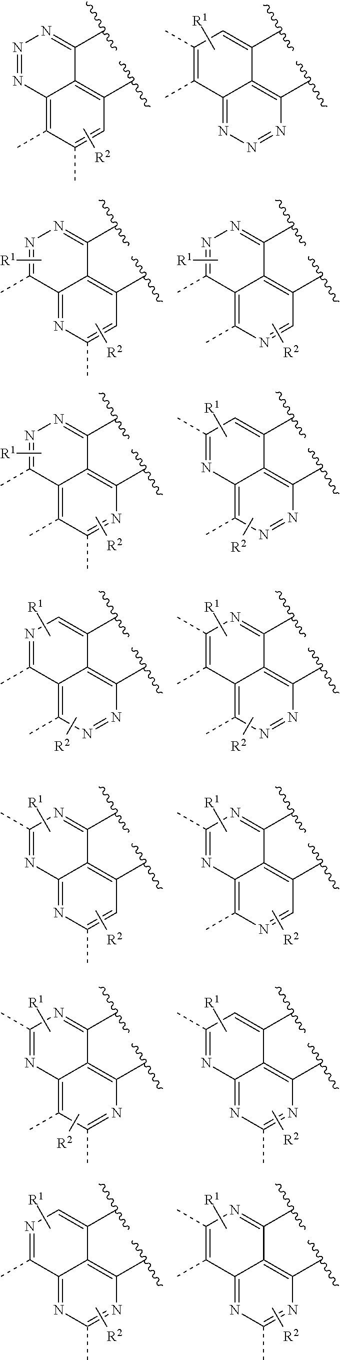

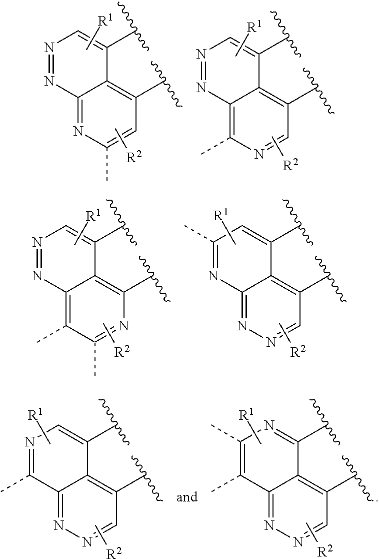

In certain embodiments

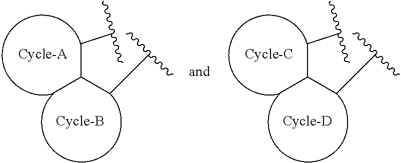

are selected from:

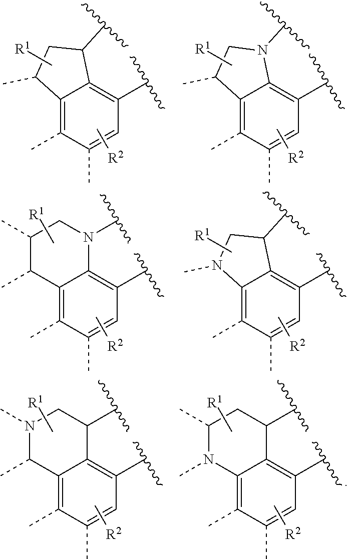

In certain embodiments

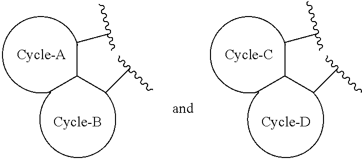

are selected from:

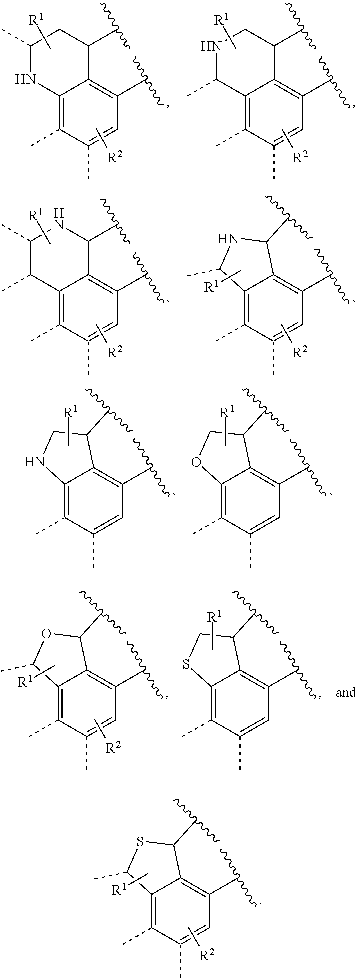

In certain embodiments

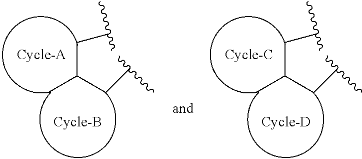

are selected from:

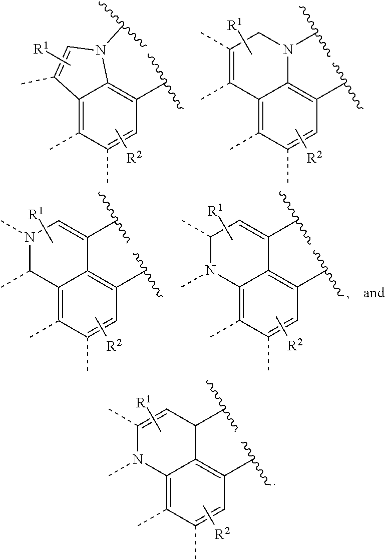

In certain embodiments

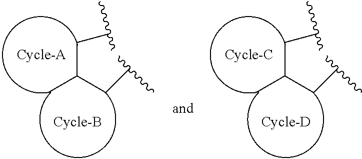

are selected from:

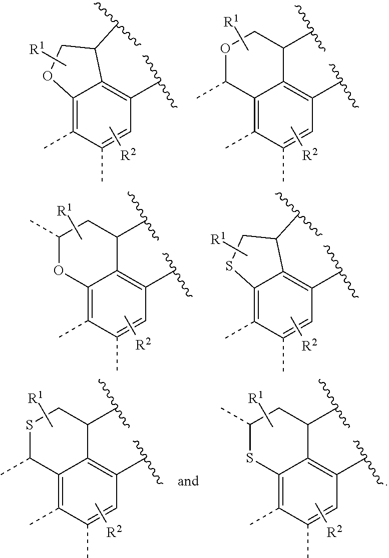

In certain embodiments

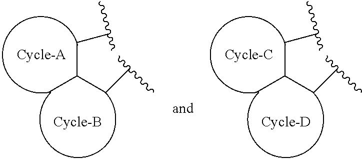

are selected from:

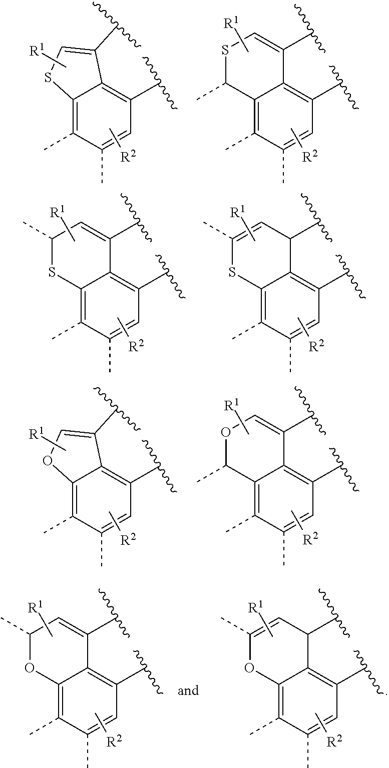

In certain embodiments

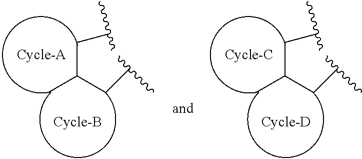

are selected from:

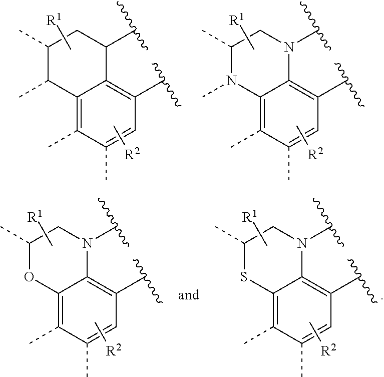

In certain embodiments

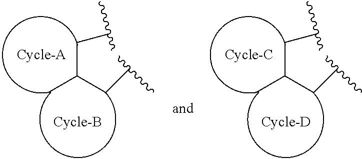

are selected from:

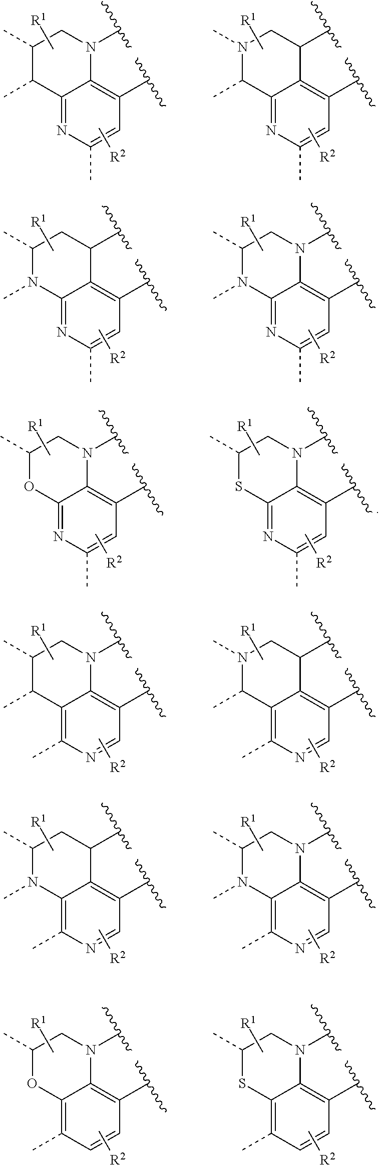

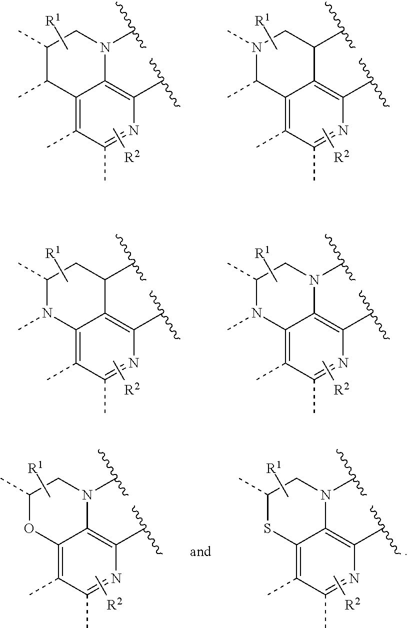

In certain embodiments

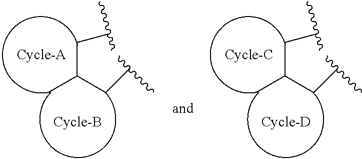

are selected from:

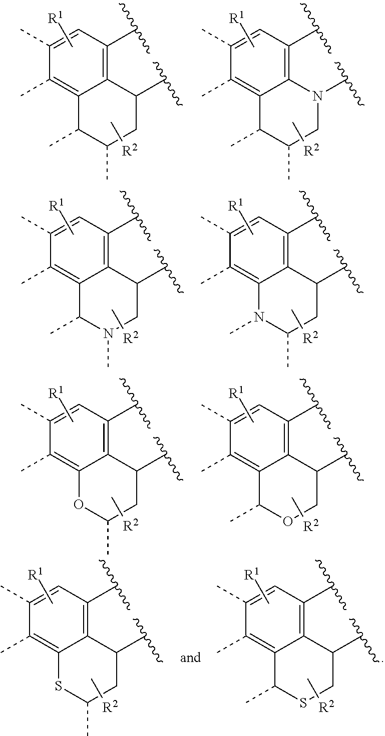

In certain embodiments

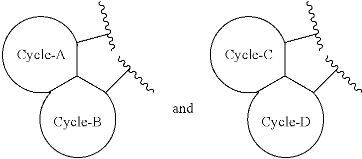

are selected from:

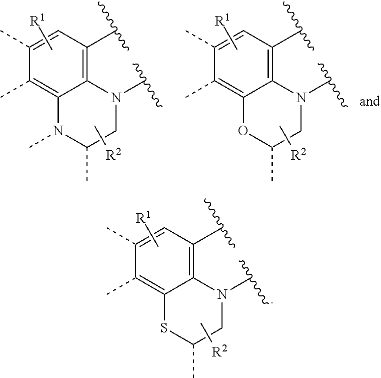

In certain embodiments

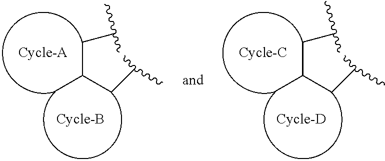

are selected from:

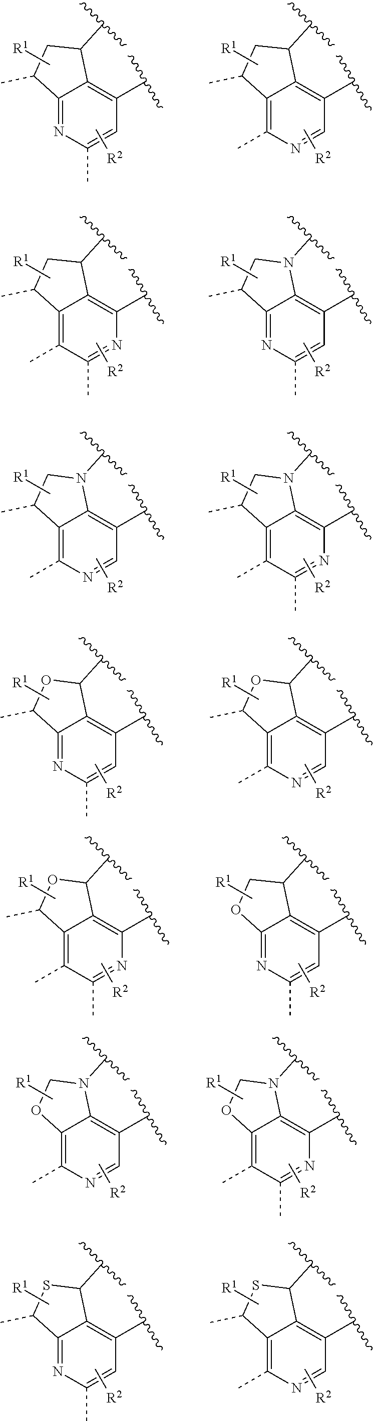

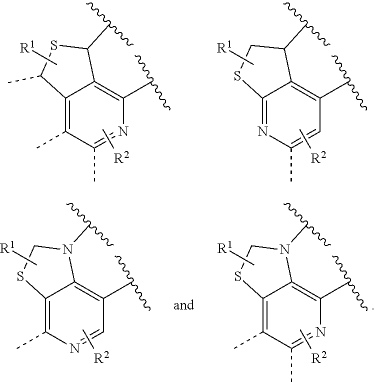

In certain embodiments

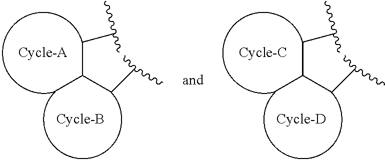

are selected from:

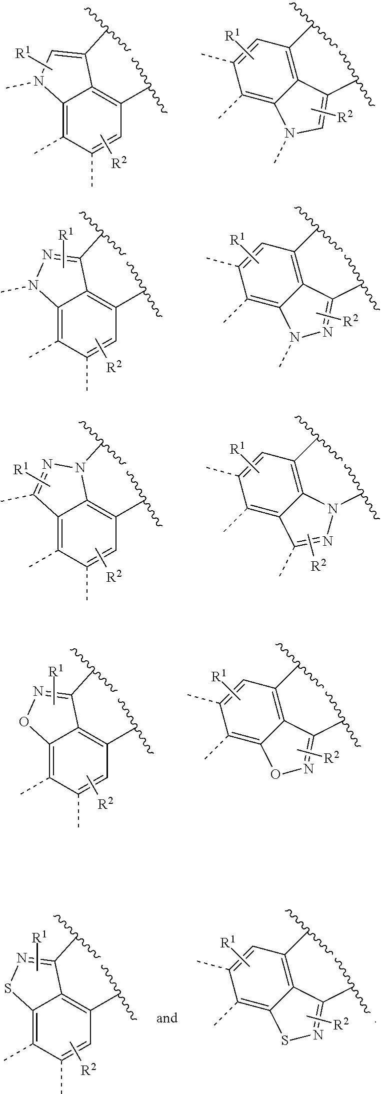

In certain embodiments

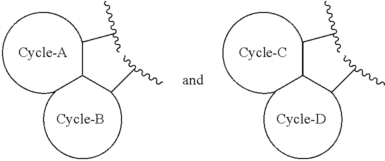

are selected from:

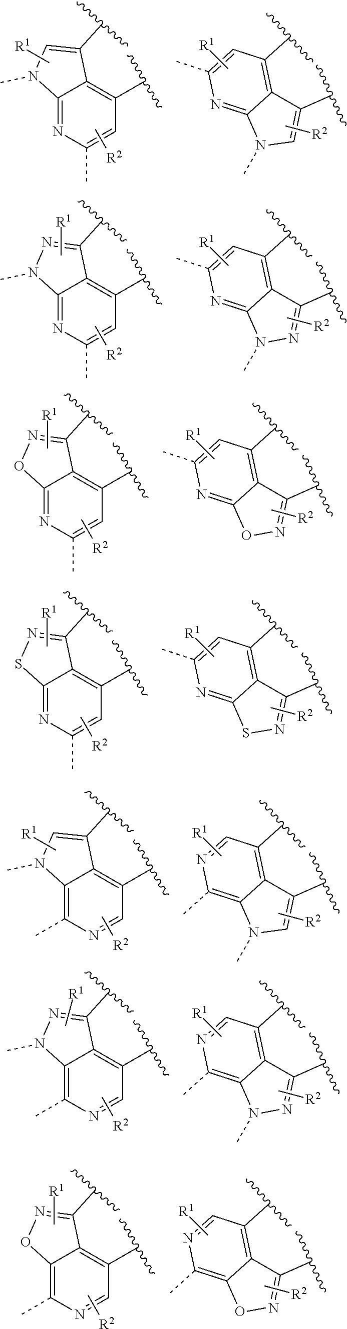

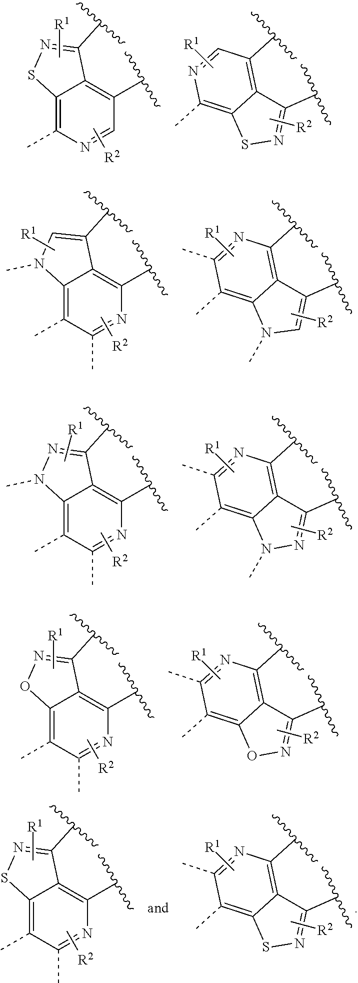

In certain embodiments

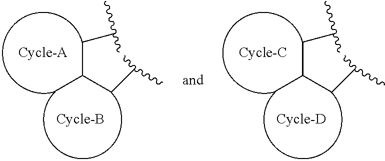

are selected from:

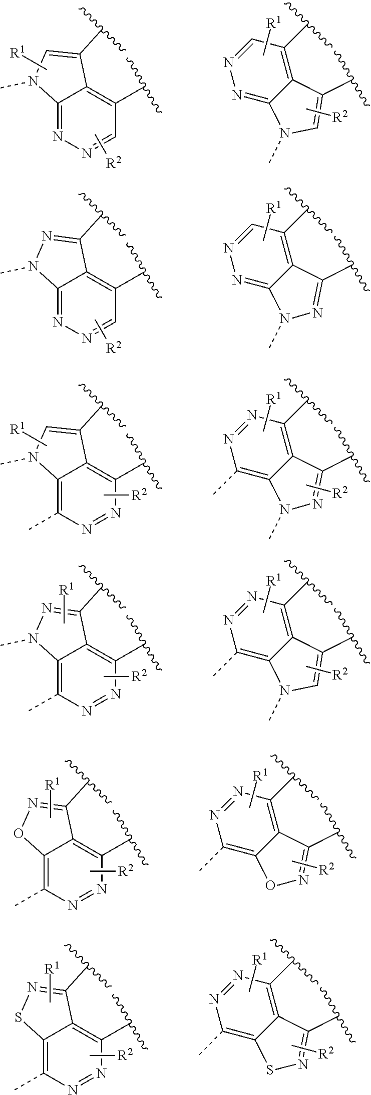

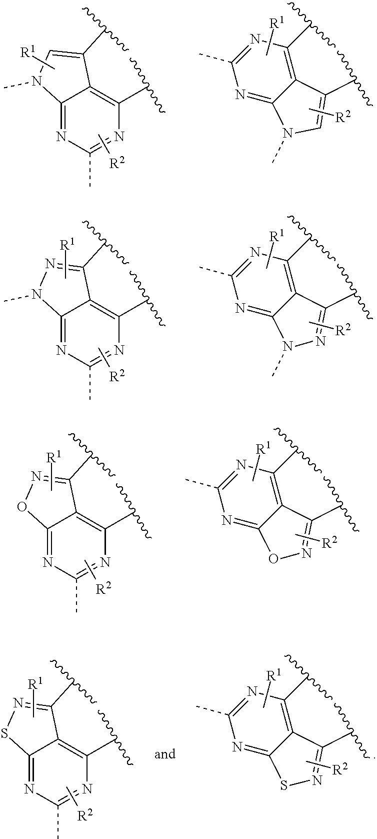

In certain embodiments

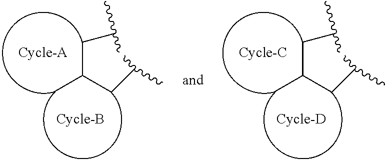

are selected from:

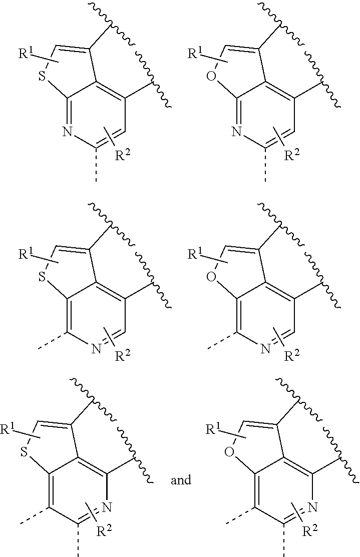

In certain embodiments

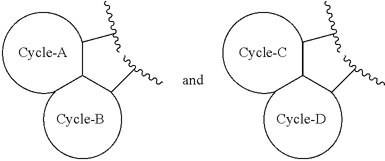

are selected from:

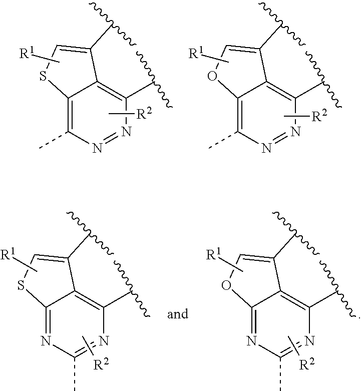

In certain embodiments

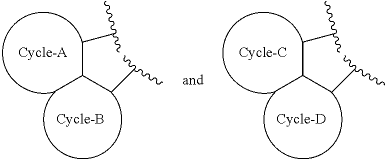

are selected from:

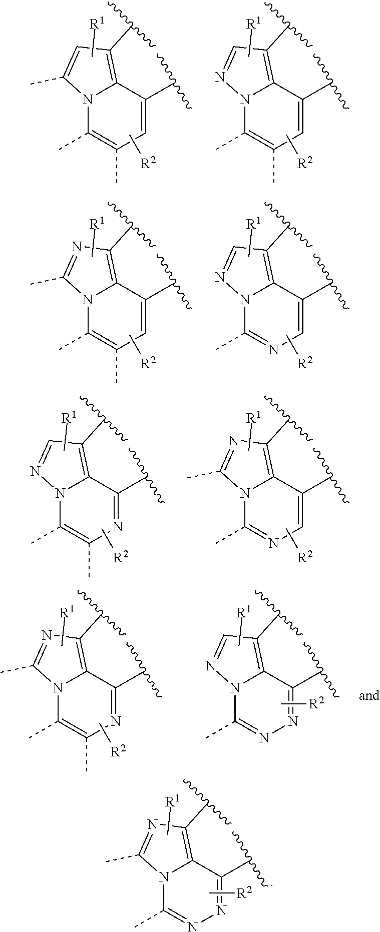

In certain embodiments

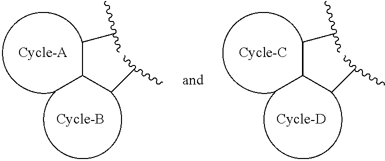

are selected from:

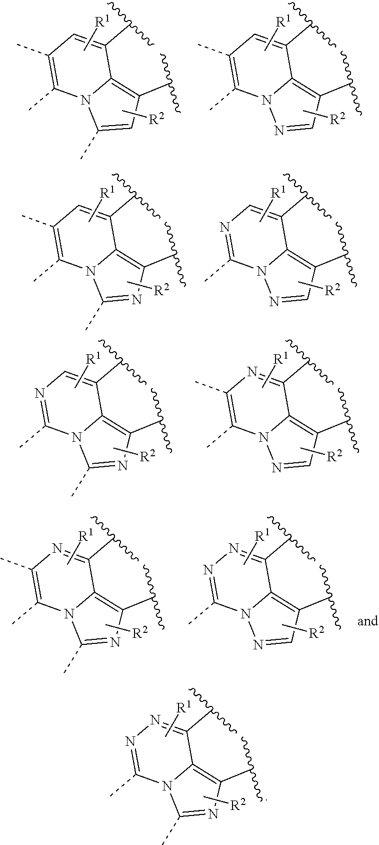

In certain embodiments

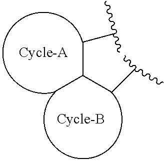 [SMILES: *CC(C)C*]

are selected from:

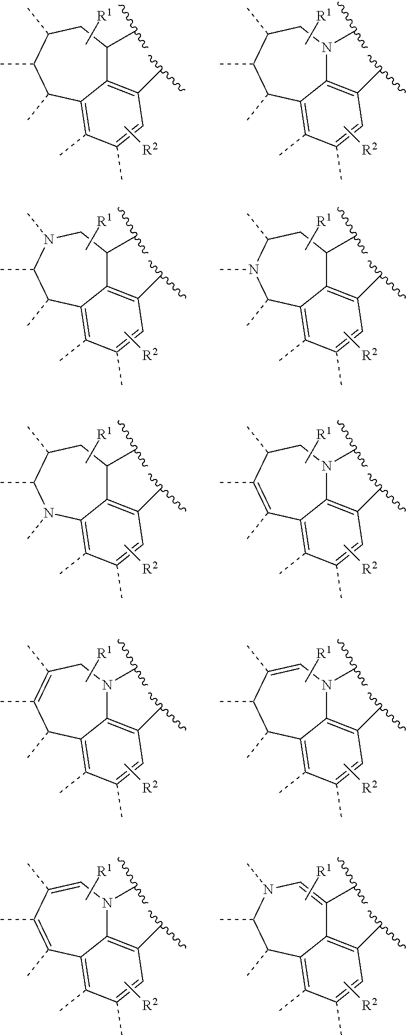

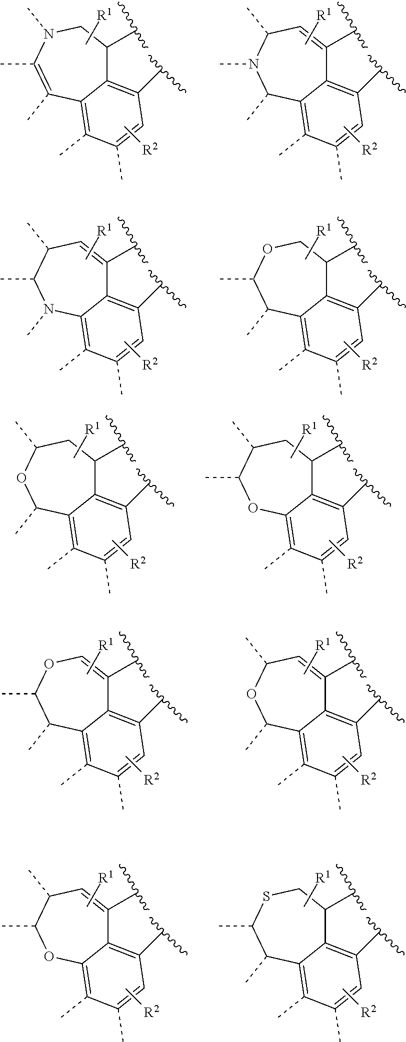

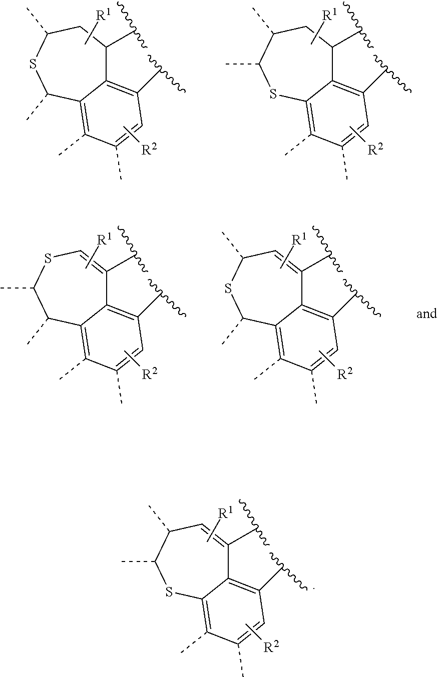

In certain embodiments

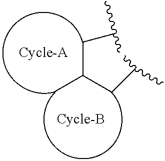 [SMILES: *CC(C)C*]

are selected from:

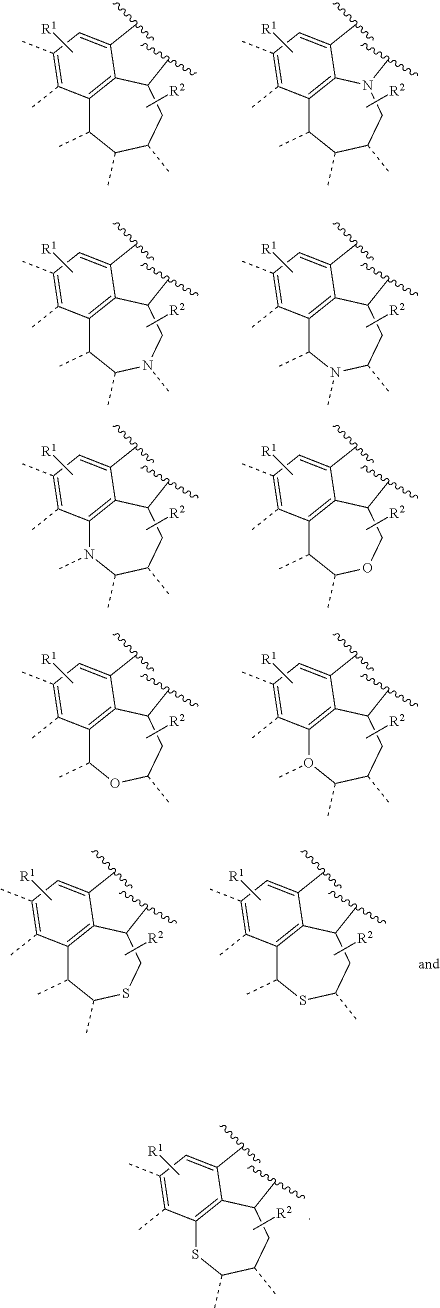

In certain embodiments

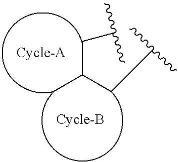 [SMILES: *CC(C)C*]

are selected from:

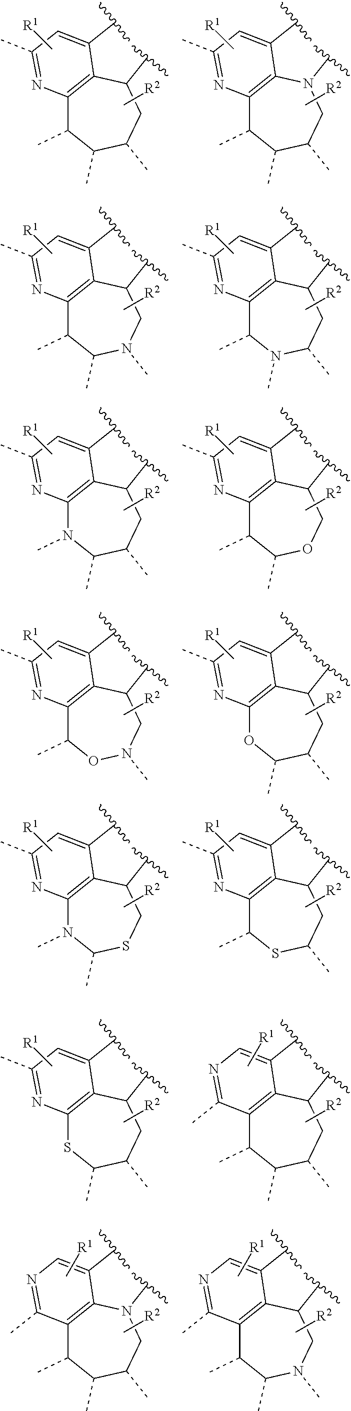

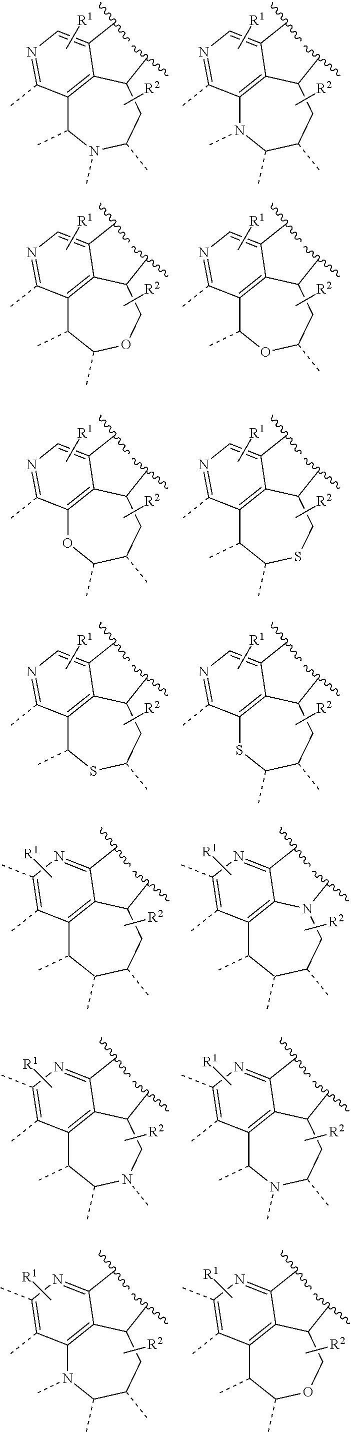

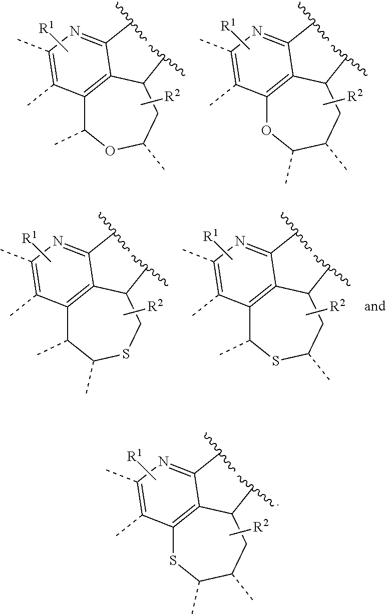

In certain embodiments

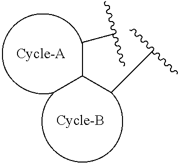 [SMILES: *CC(C)C*]

are selected from:

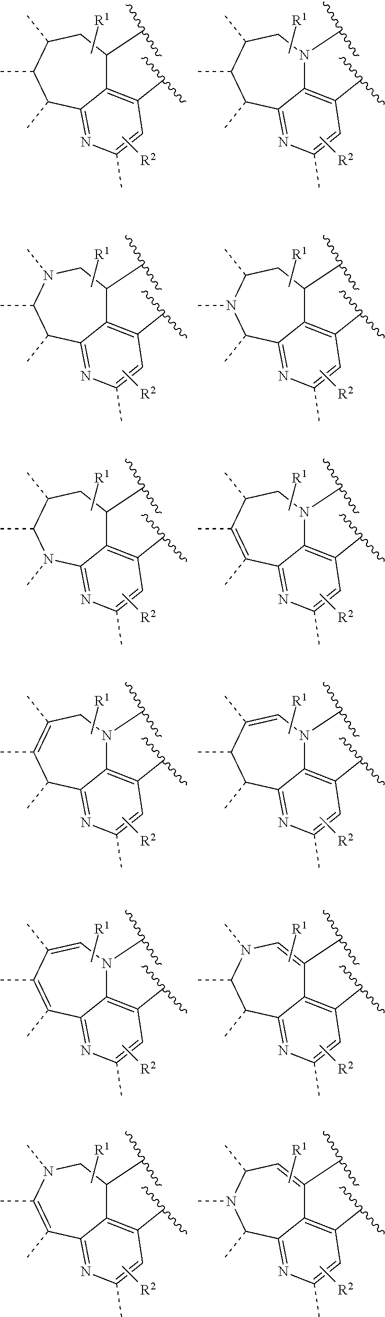

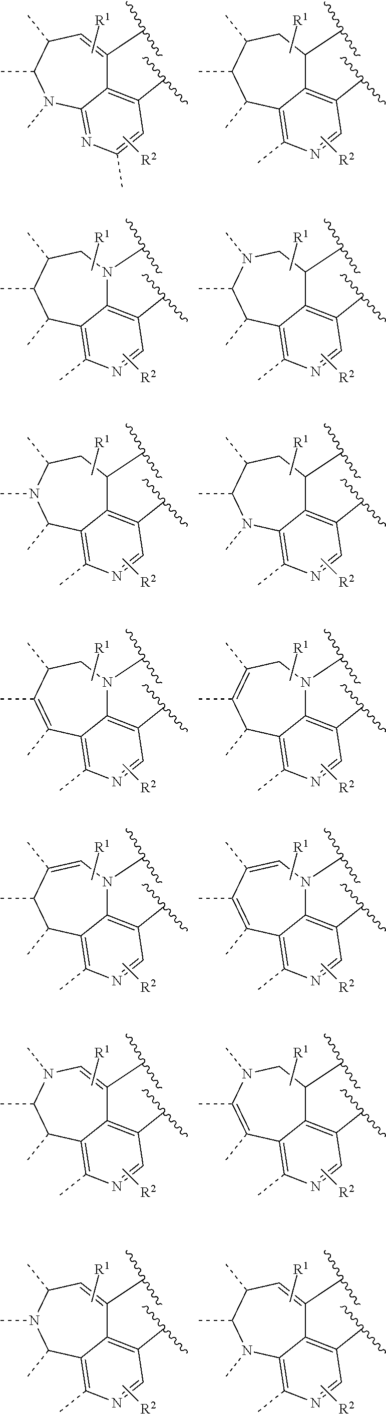

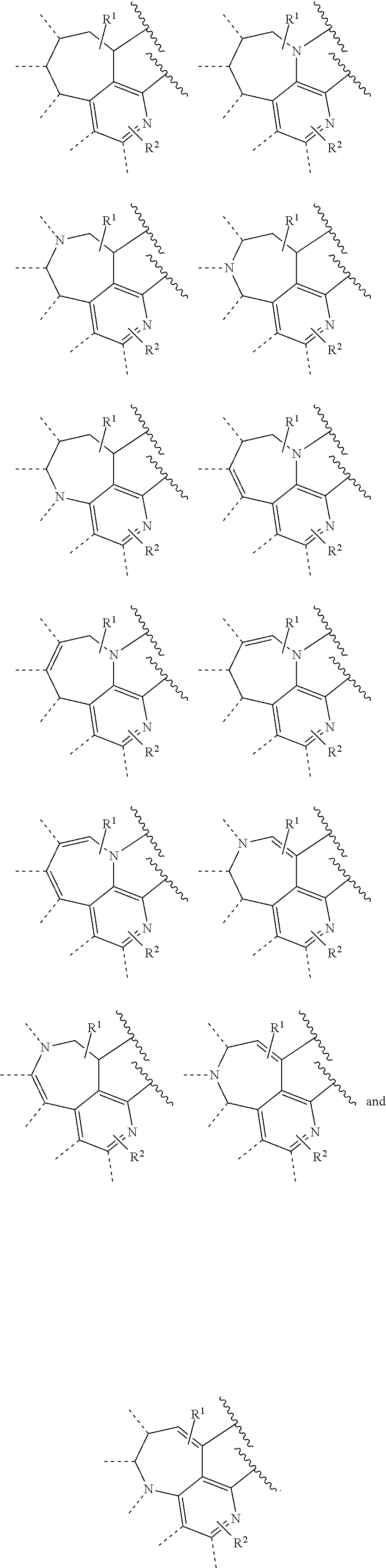

In certain embodiments

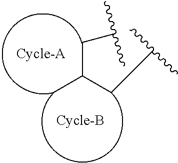 [SMILES: *CC(C)C*]

are selected from:

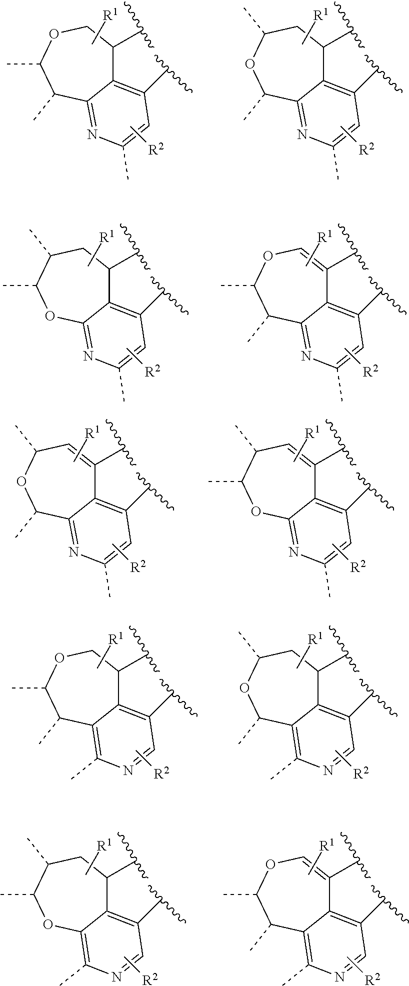

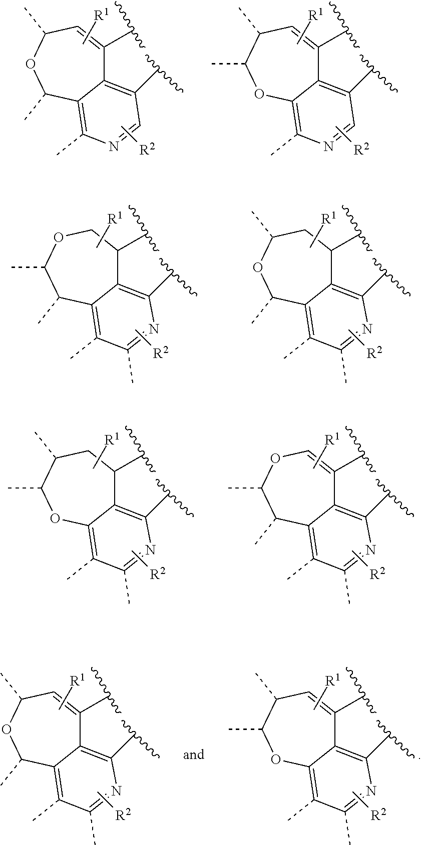

In certain embodiments

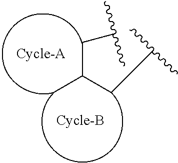 [SMILES: *CC(C)C*]

are selected from:

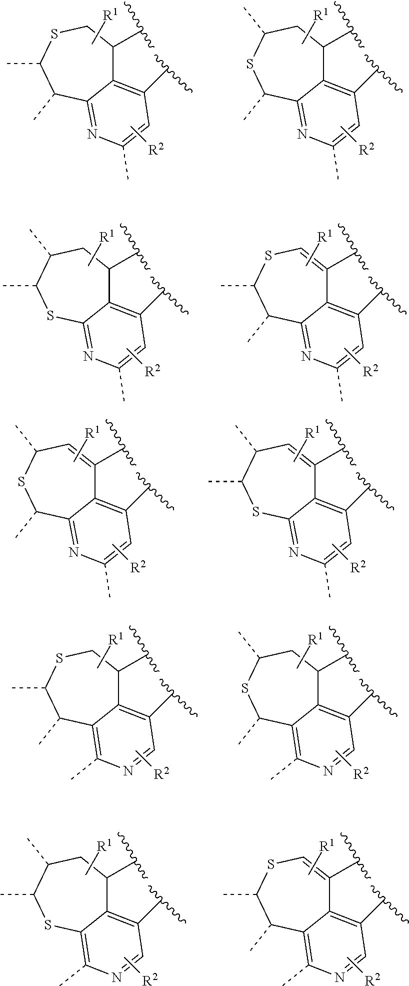

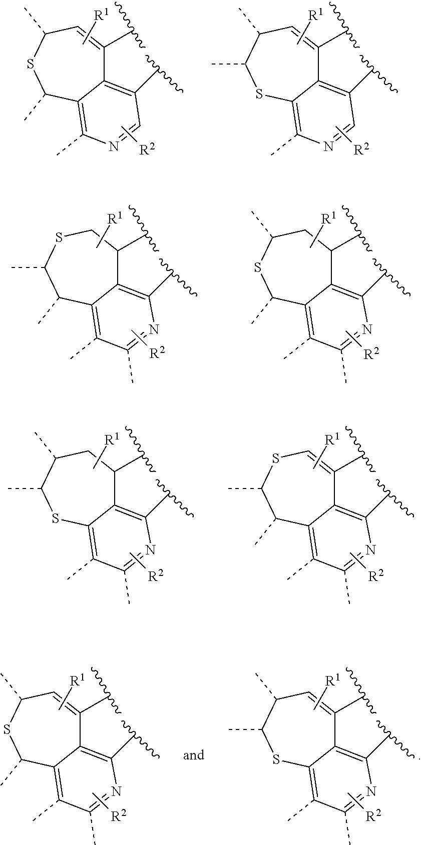

In certain embodiments

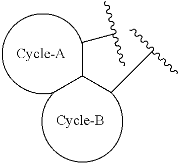 [SMILES: *CC(C)C*]

are selected from:

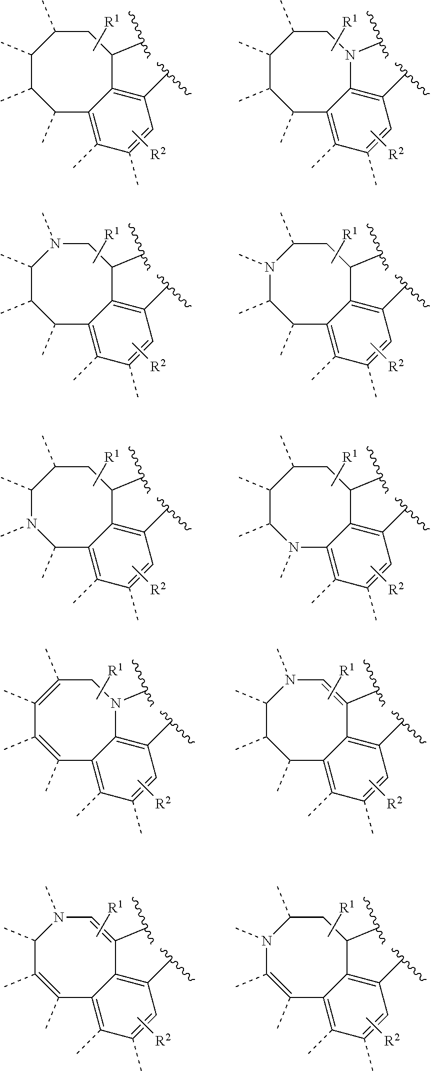

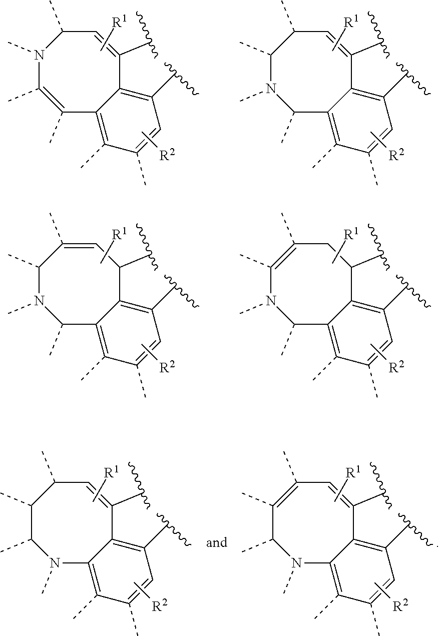

In certain embodiments

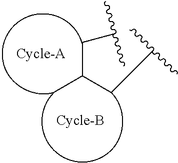 [SMILES: *CC(C)C*]

are selected from:

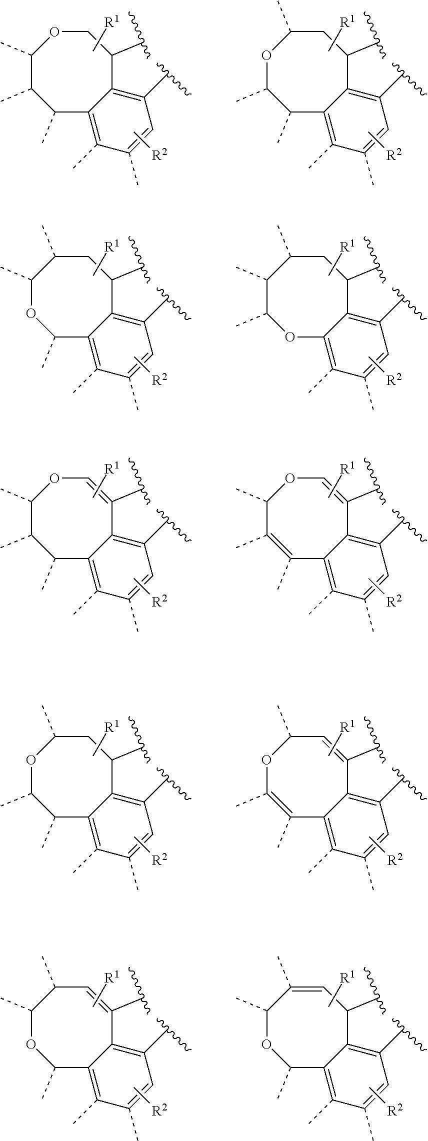

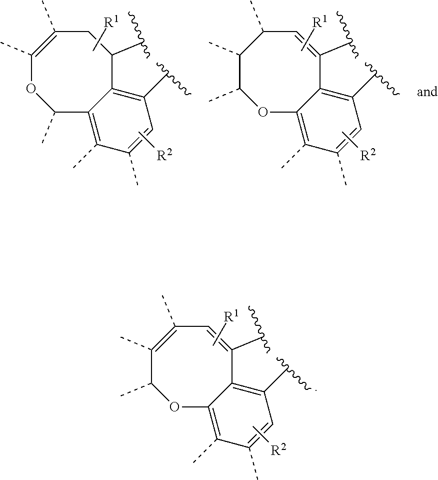

In certain embodiments

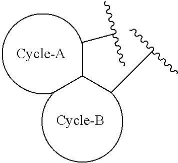 [SMILES: *CC(C)C*]

are selected from:

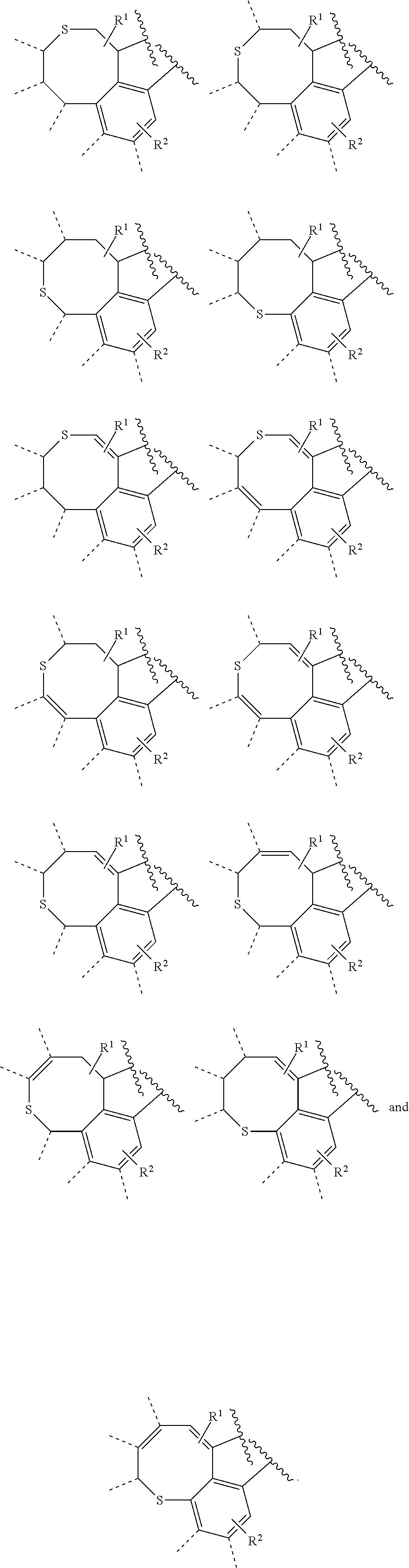

In certain embodiments

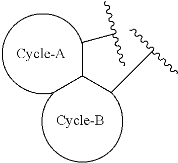 [SMILES: *CC(C)C*]

are selected from:

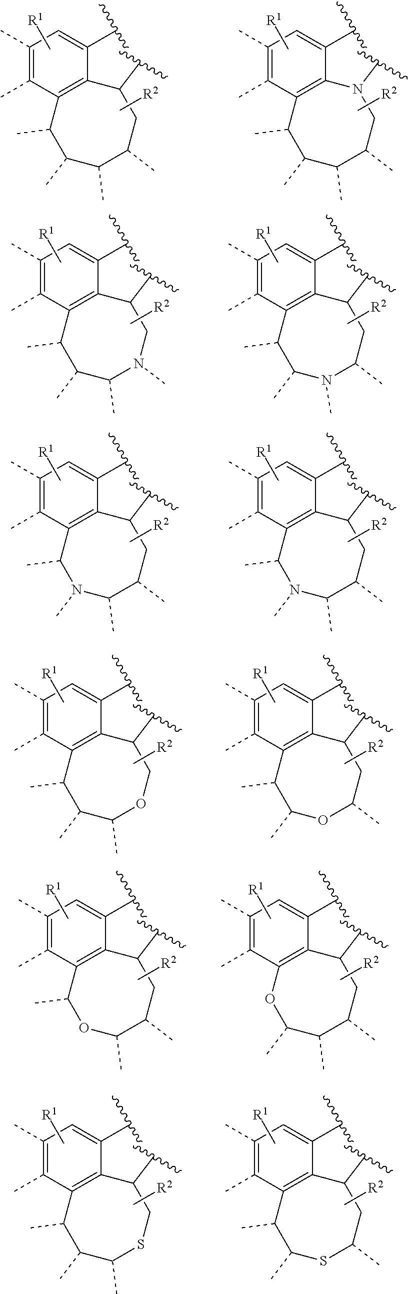

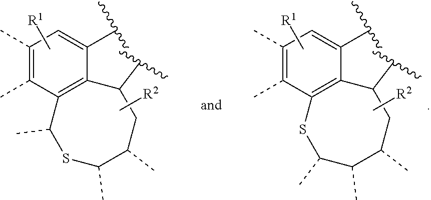

In certain embodiments

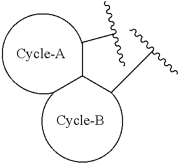 [SMILES: *CC(C)C*]

are selected from:

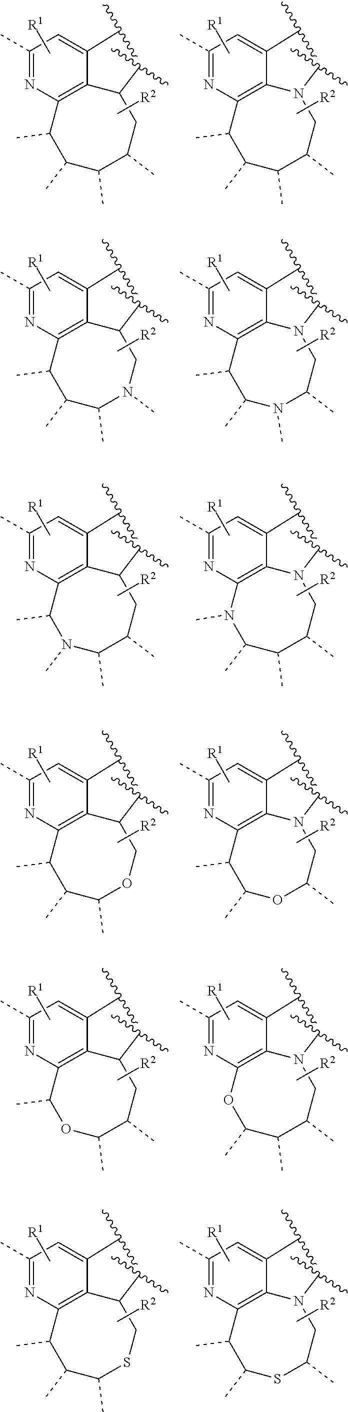

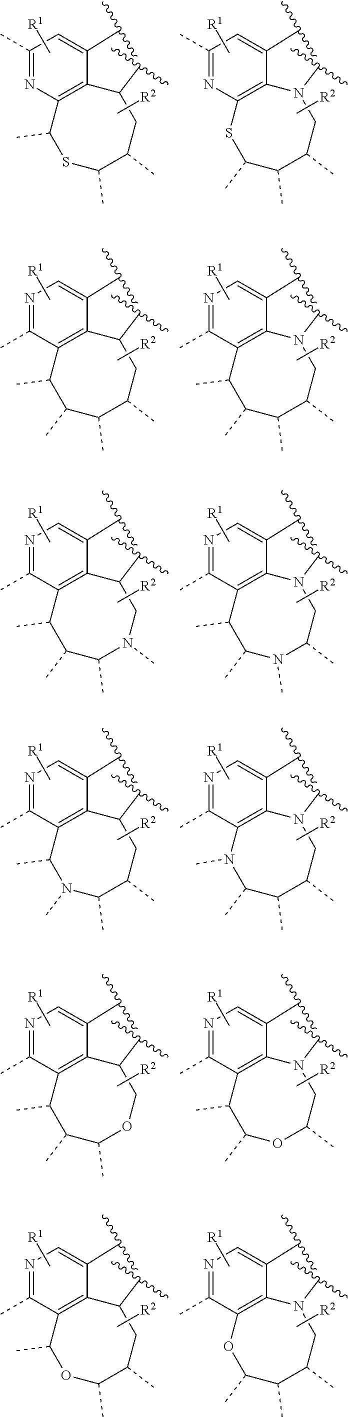

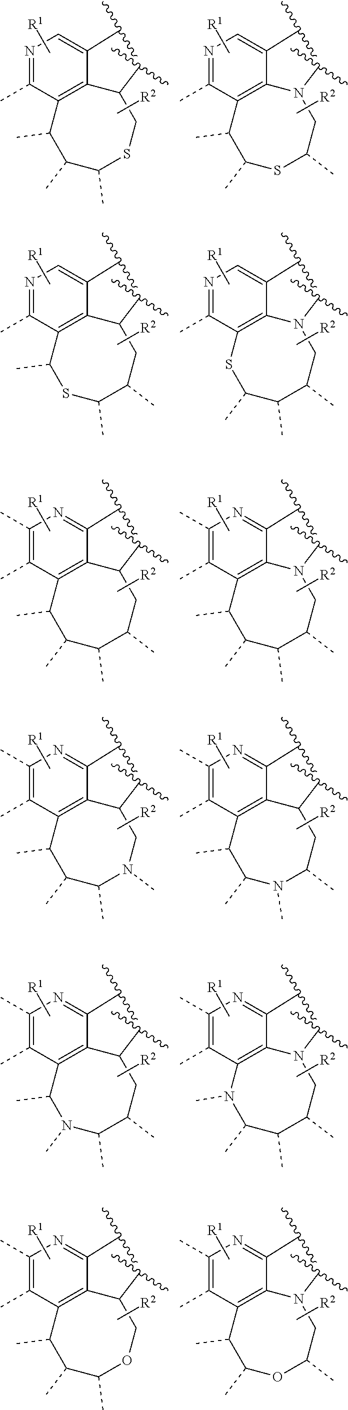

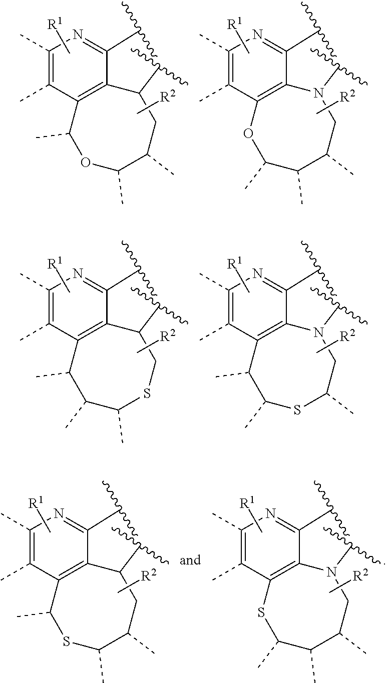

In certain embodiments

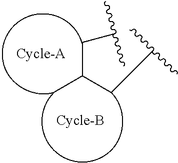 [SMILES: *CC(C)C*]

are selected from:

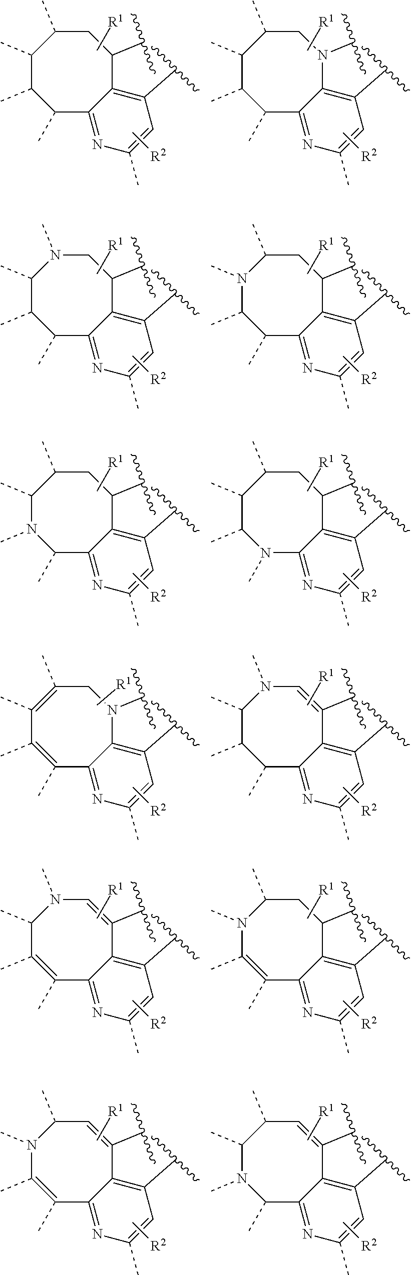

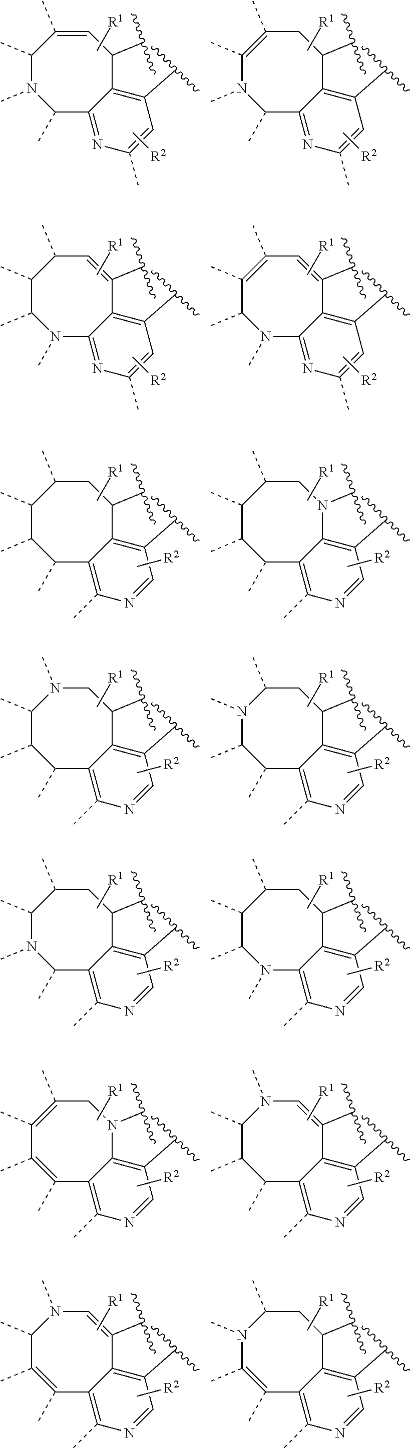

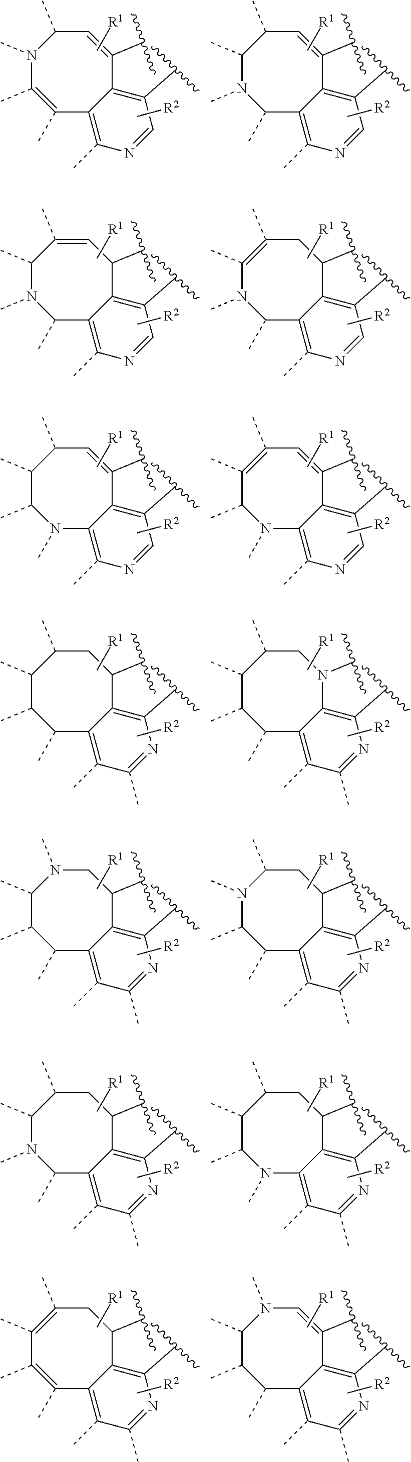

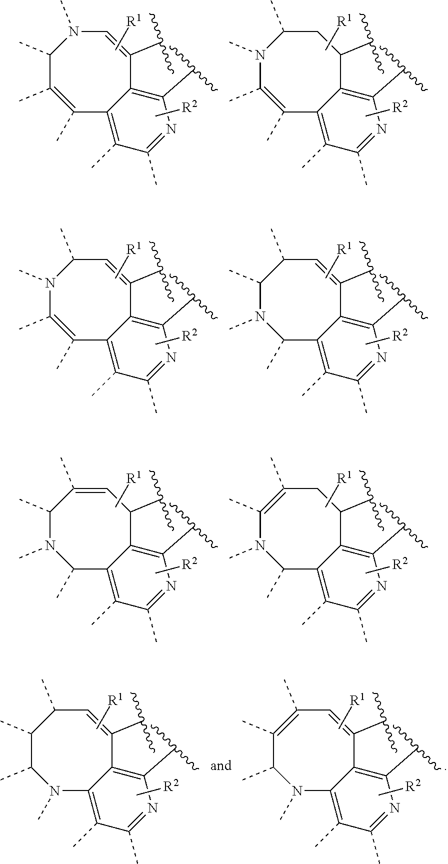

In certain embodiments

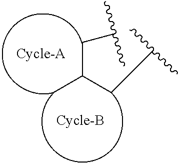 [SMILES: *CC(C)C*]

are selected from:

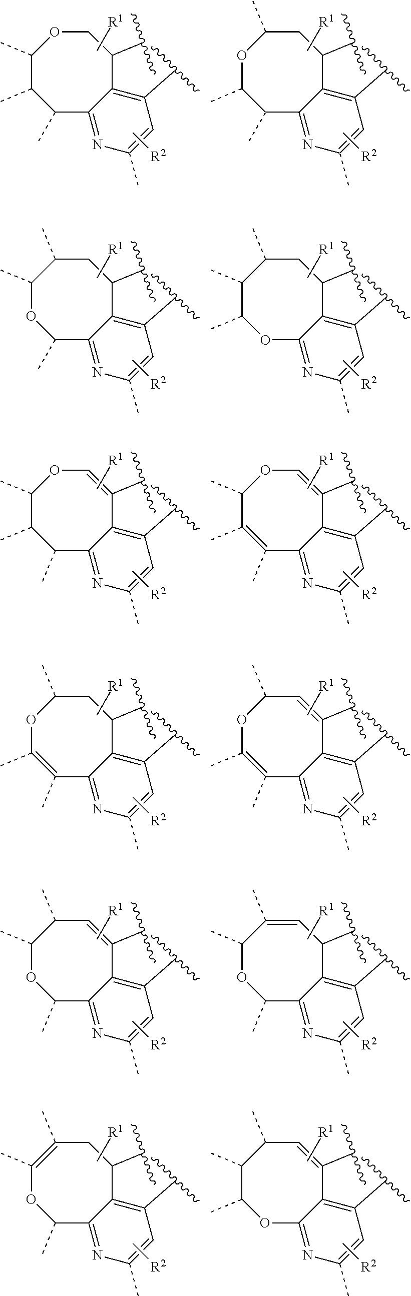

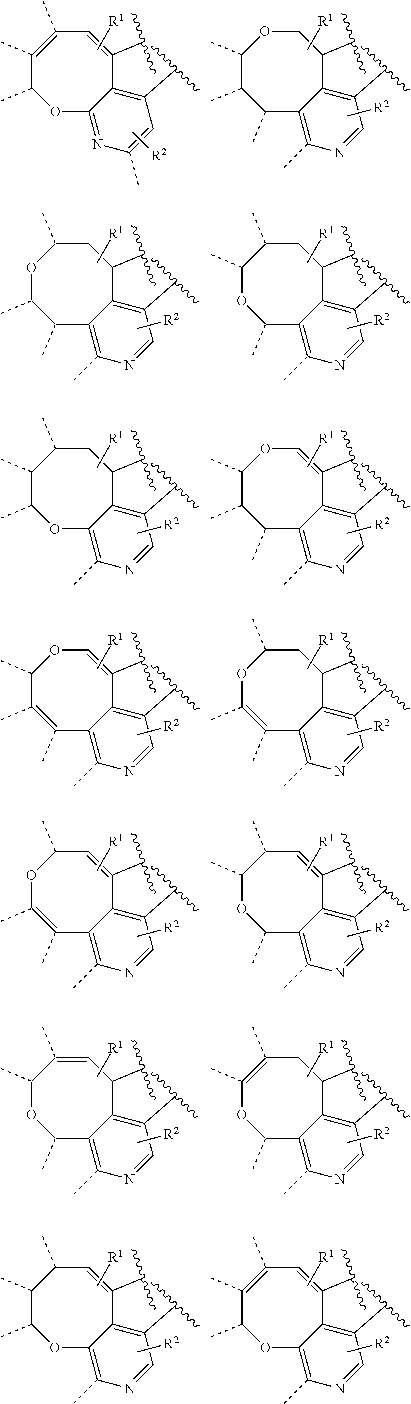

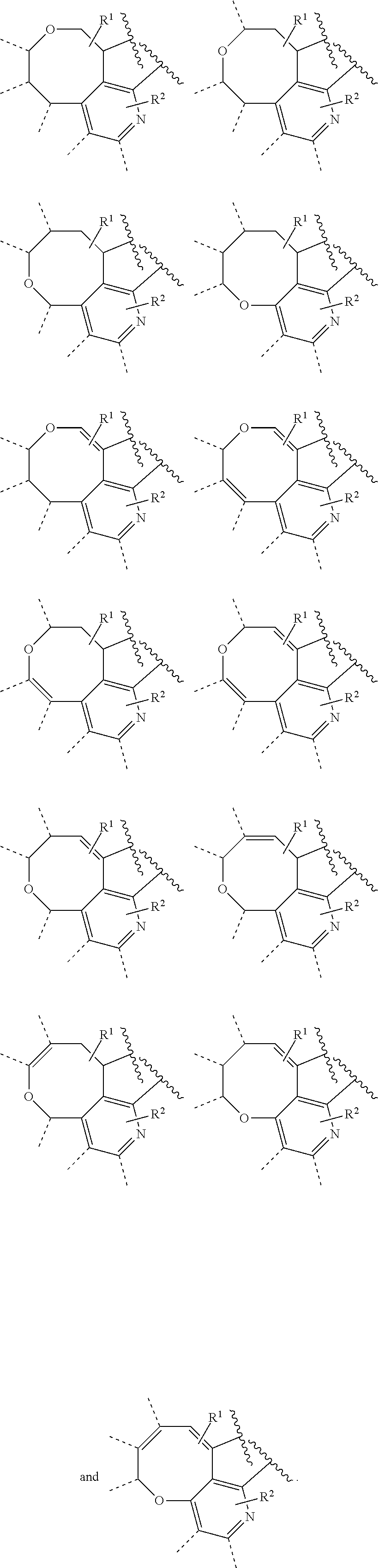

In certain embodiments

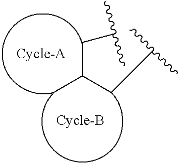 [SMILES: *CC(C)C*]

are selected from:

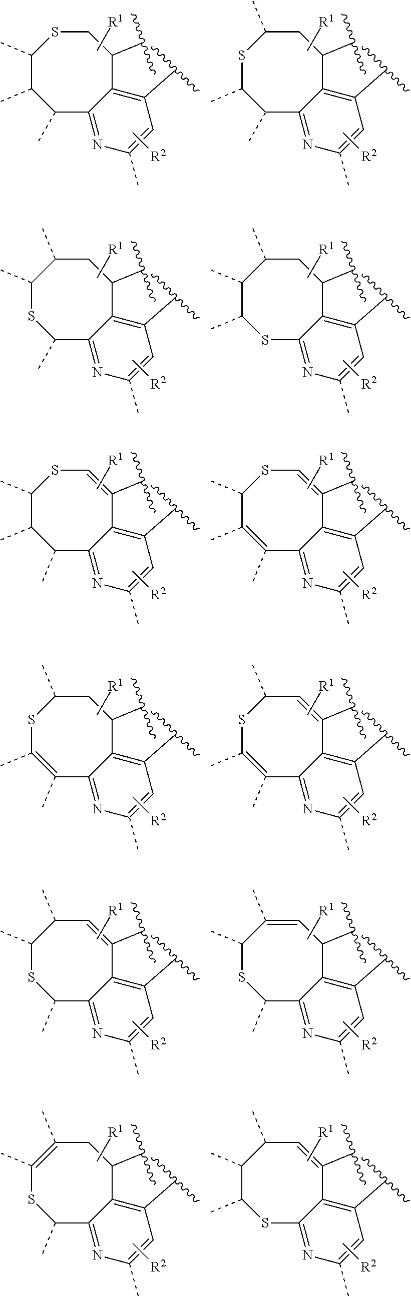

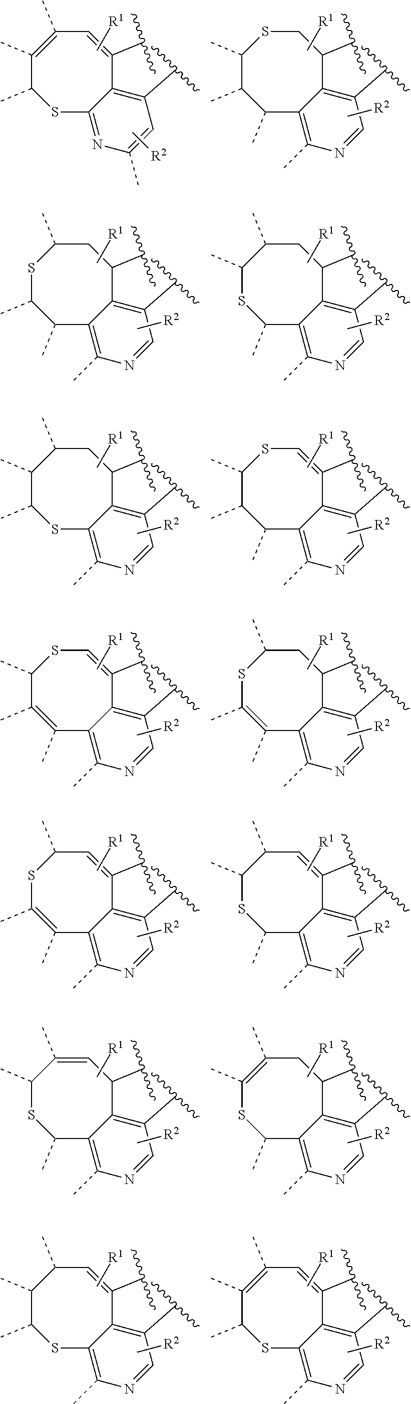

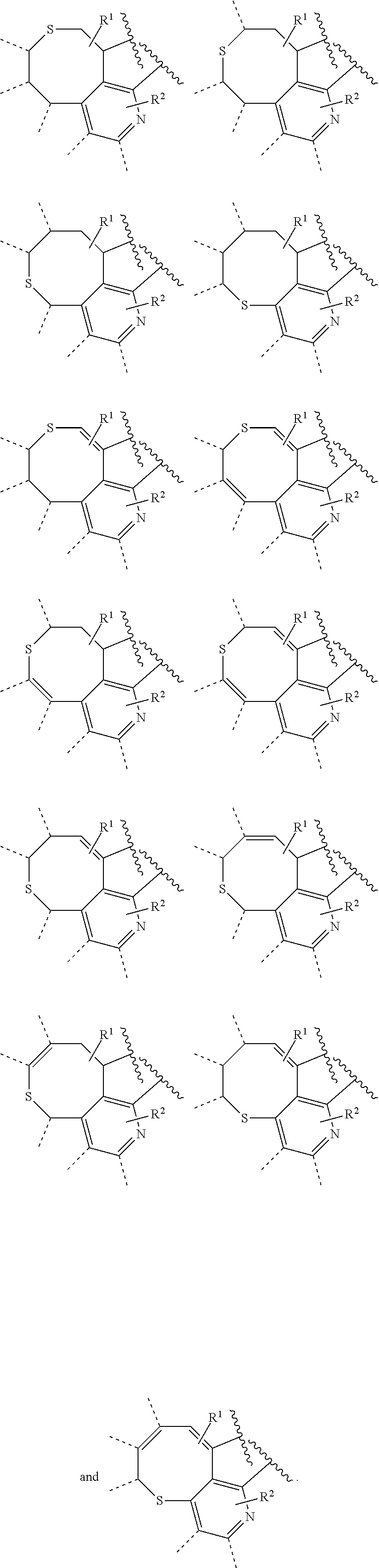

In certain embodiments

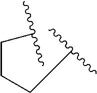 [SMILES: *CCC*]

for

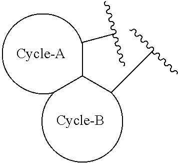 [SMILES: *CC(C)C*]

is selected from:

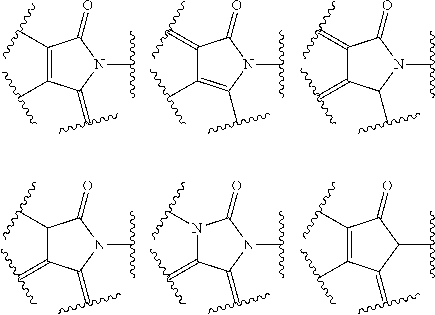 [SMILES: *C1=C(*)C(=O)N(*)C1=* ; *C1=C(*)N(*)C(=O)C1=* ; *C1C(=*)C(=*)C(=O)N1* ; *C1C(=*)C(=*)N(*)C1=O ; *n1c(=*)c(=*)n(*)c1=O ; *C1=C(*)C(=O)C(*)C1=*]

In certain embodiments

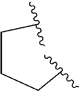 [SMILES: *CCC*]

within

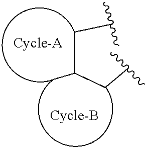 [SMILES: *CC(C)C*]

is selected from:

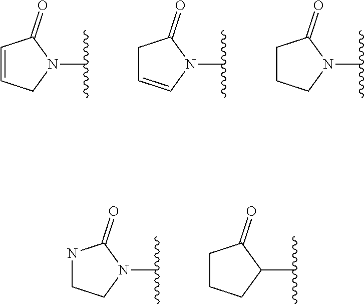 [SMILES: *N1CC=CC1=O ; *N1C=CCC1=O ; *N1CCCC1=O ; *N1CCNC1=O ; *C1CCCC1=O]

for example, when

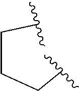 [SMILES: *CCC*]

is

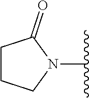 [SMILES: *N1CCCC1=O]

then

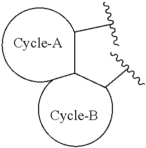 [SMILES: *CC(C)C*]

is

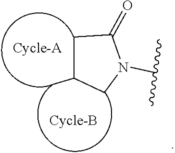 [SMILES: *N1CC(C)CC1=O]

### Cycle-A

In certain embodiments

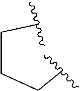 [SMILES: *CCC*]

within

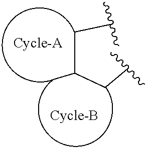 [SMILES: *CC(C)C*]

is selected from:

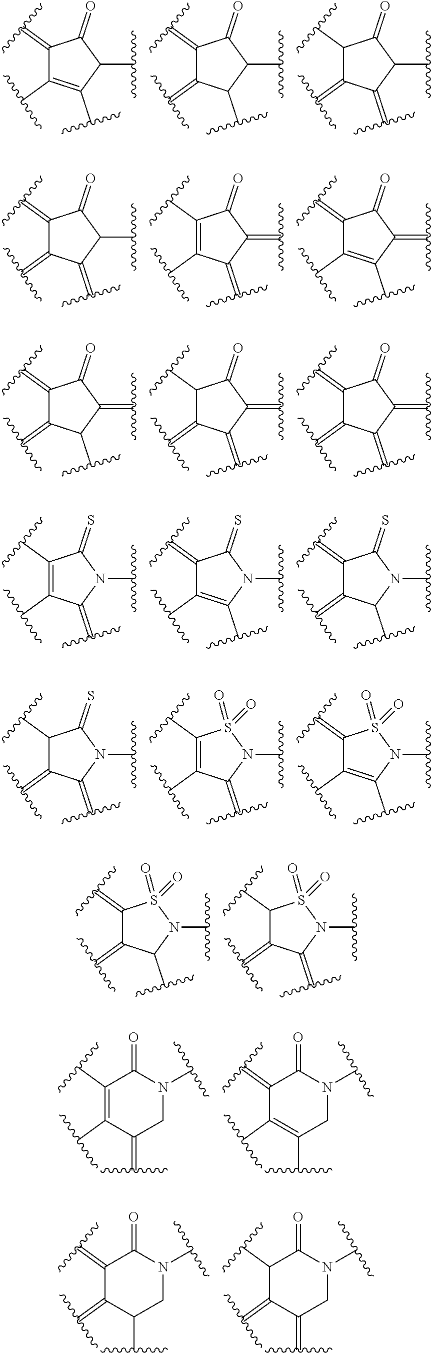

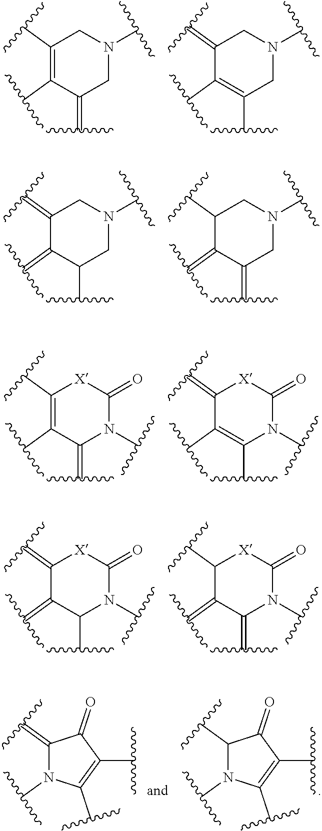

In certain embodiments

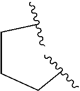 [SMILES: *CCC*]

for

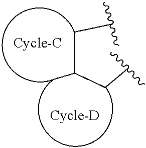 [SMILES: *CC(C)C*]

is selected from:

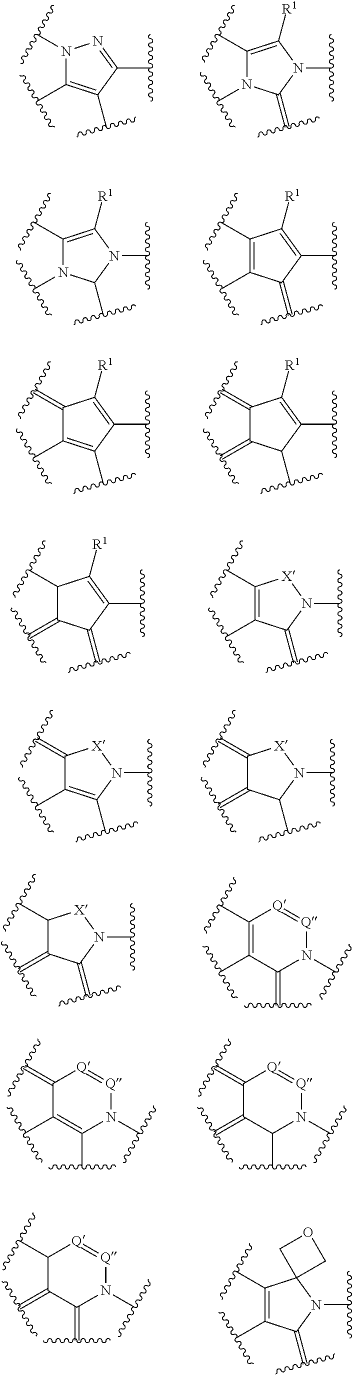

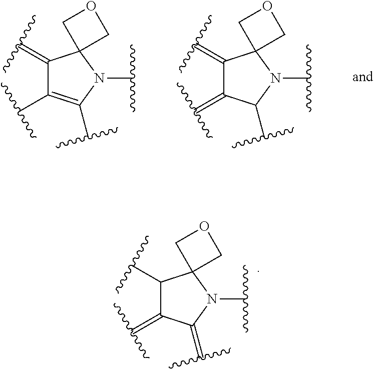 [SMILES: *C1=C(*)N(*)C2(COC2)C1=* ; *C1C(=*)C(=*)C2(COC2)N1* ; *C1C(=*)C(=*)N(*)C12COC2]

In the structures herein the structure

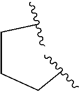 [SMILES: *CCC*]

refers to the cycloalkyl, heterocyclic, aryl, or heteroaryl ring fused to either Cycle-A and Cycle-B or Cycle-C and Cycle-D. This is demonstrated by the non-limiting examples of:

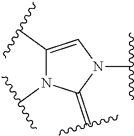 [SMILES: *C1=CN(*)C(=*)N1*]

refers to

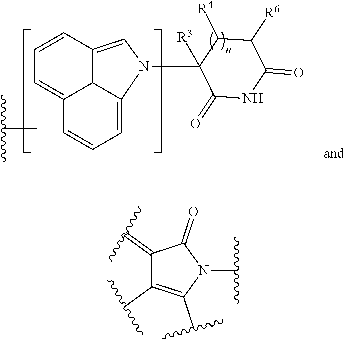

refers to

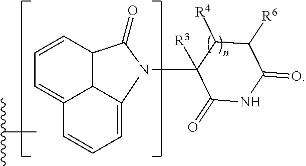

As used here

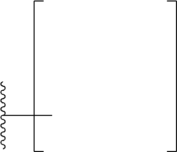

depicts a connection point of the Tricyclic Cereblon Ligand to any position on the tricyclic ring as allowed by valence.

In certain embodiment

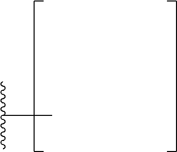

is bound to the first available position (counting counter clockwise) on Cycle-A or Cycle-C. For example, in this embodiment

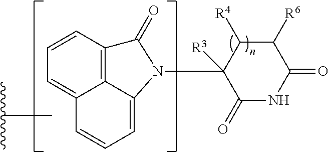

is

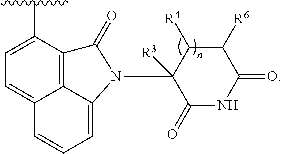

In certain embodiments

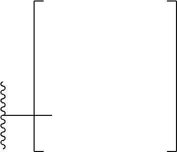

is bound to the second available position (counting counter clockwise) on Cycle-A or Cycle-C. For example, in this embodiment

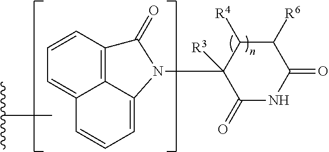

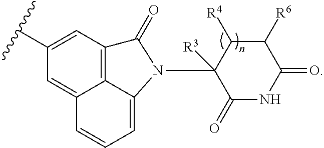

In certain embodiments

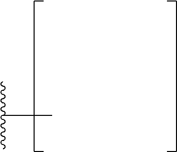

is bound to the third available position (counting counter clockwise) on Cycle-A or Cycle-C. For example, in this embodiment

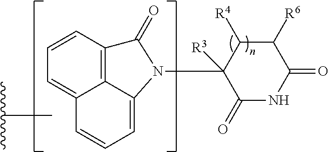

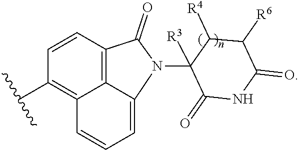

In certain embodiments

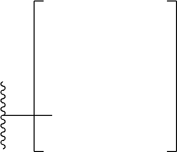

is bound to the first available position (counting counter clockwise) on Cycle-B or Cycle-D. For example, in this embodiment

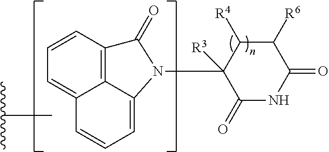

is

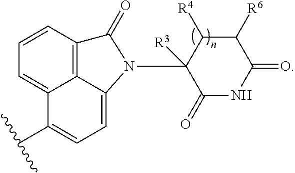

In certain embodiments

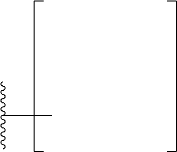

is bound to the second available position (counting counter clockwise) on Cycle-B or Cycle-D. For example, in this embodiment

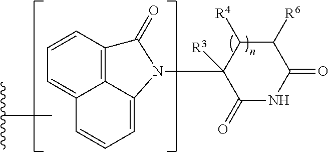

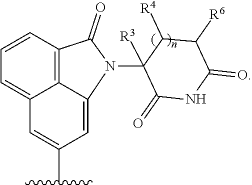

In certain embodiments

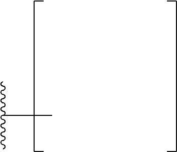

is bound to the third available position (counting counter clockwise) on Cycle-B or Cycle-D. For example, in this embodiment

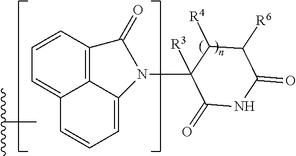

is

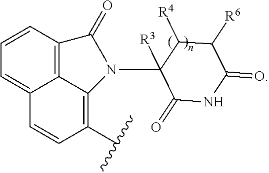

In certain embodiments

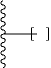

is connected at a point selected from the second or third position on Cycle-A or the first or second position on Cycle-B. For example, in this embodiment

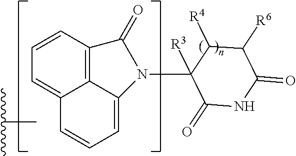

is

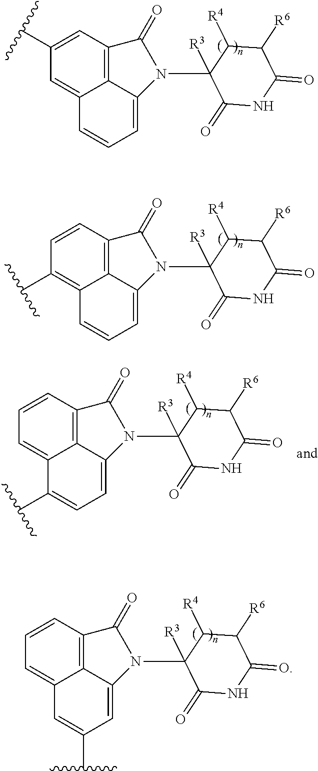

In certain embodiments Cycle-A is phenyl optionally substituted with 1, 2, or 3 substituents independently selected from R¹ as allowed by valence.

In certain embodiments Cycle-A is 5-membered heteroaryl optionally substituted with 1, 2, or 3 substituents independently selected from R¹ as allowed by valence.

In certain embodiments Cycle-A is 6-membered heteroaryl optionally substituted with 1, 2, or 3 substituents independently selected from R¹ as allowed by valence.

In certain embodiments Cycle-A is 5-membered heterocycle optionally substituted with 1, 2, or 3 substituents independently selected from R¹ as allowed by valence.

In certain embodiments Cycle-A is 6-membered heterocycle optionally substituted with 1, 2, or 3 substituents independently selected from R¹ as allowed by valence.

In certain embodiments Cycle-A is 7-membered heterocycle optionally substituted with 1, 2, or 3 substituents independently selected from R¹ as allowed by valence.

In certain embodiments Cycle-A is 8-membered heterocycle optionally substituted with 1, 2, or 3 substituents independently selected from R¹ as allowed by valence.

In certain embodiments Cycle-A is 5-membered cycloalkyl optionally substituted with 1, 2, or 3 substituents independently selected from R¹ as allowed by valence.

In certain embodiments Cycle-A is 6-membered cycloalkyl optionally substituted with 1, 2, or 3 substituents independently selected from R¹ as allowed by valence.

In certain embodiments Cycle-A is 7-membered cycloalkyl optionally substituted with 1, 2, or 3 substituents independently selected from R¹ as allowed by valence.

In certain embodiments Cycle-A is 8-membered cycloalkyl optionally substituted with 1, 2, or 3 substituents independently selected from R¹ as allowed by valence.

In certain embodiments Cycle-B is phenyl optionally substituted with 1, 2, or 3 substituents independently selected from R² as allowed by valence.

In certain embodiments Cycle-B is 5-membered heteroaryl optionally substituted with 1, 2, or 3 substituents independently selected from R² as allowed by valence.

In certain embodiments Cycle-B is 6-membered heteroaryl optionally substituted with 1, 2, or 3 substituents independently selected from R² as allowed by valence.

In certain embodiments Cycle-B is 5-membered heterocycle optionally substituted with 1, 2, or 3 substituents independently selected from R² as allowed by valence.

In certain embodiments Cycle-B is 6-membered heterocycle optionally substituted with 1, 2, or 3 substituents independently selected from R² as allowed by valence.

In certain embodiments Cycle-B is 7-membered heterocycle optionally substituted with 1, 2, or 3 substituents independently selected from R² as allowed by valence.

In certain embodiments Cycle-B is 8-membered heterocycle optionally substituted with 1, 2, or 3 substituents independently selected from R² as allowed by valence.

In certain embodiments Cycle-B is 5-membered cycloalkyl optionally substituted with 1, 2, or 3 substituents independently selected from R² as allowed by valence.

In certain embodiments Cycle-B is 6-membered cycloalkyl optionally substituted with 1, 2, or 3 substituents independently selected from R² as allowed by valence.

In certain embodiments Cycle-B is 7-membered cycloalkyl optionally substituted with 1, 2, or 3 substituents independently selected from R² as allowed by valence.

In certain embodiments Cycle-B is 8-membered cycloalkyl optionally substituted with 1, 2, or 3 substituents independently selected from R² as allowed by valence.

In certain embodiments Cycle-C is phenyl optionally substituted with 1, 2, or 3 substituents independently selected from R¹ as allowed by valence.

In certain embodiments Cycle-C is 5-membered heteroaryl optionally substituted with 1, 2, or 3 substituents independently selected from R¹ as allowed by valence.

In certain embodiments Cycle-C is 6-membered heteroaryl optionally substituted with 1, 2, or 3 substituents independently selected from R¹ as allowed by valence.

In certain embodiments Cycle-C is 5-membered heterocycle optionally substituted with 1, 2, or 3 substituents independently selected from R¹ as allowed by valence.

In certain embodiments Cycle-C is 6-membered heterocycle optionally substituted with 1, 2, or 3 substituents independently selected from R¹ as allowed by valence.

In certain embodiments Cycle-C is 5-membered cycloalkyl optionally substituted with 1, 2, or 3 substituents independently selected from R¹ as allowed by valence.

In certain embodiments Cycle-C is 6-membered cycloalkyl optionally substituted with 1, 2, or 3 substituents independently selected from R¹ as allowed by valence.

In certain embodiments Cycle-D is phenyl optionally substituted with 1, 2, or 3 substituents independently selected from R² as allowed by valence.

In certain embodiments Cycle-D is 5-membered heteroaryl optionally substituted with 1, 2, or 3 substituents independently selected from R² as allowed by valence.

In certain embodiments Cycle-D is 6-membered heteroaryl optionally substituted with 1, 2, or 3 substituents independently selected from R² as allowed by valence.

In certain embodiments Cycle-D is 5-membered heterocycle optionally substituted with 1, 2, or 3 substituents independently selected from R² as allowed by valence.

In certain embodiments Cycle-D is 6-membered heterocycle optionally substituted with 1, 2, or 3 substituents independently selected from R² as allowed by valence.

In certain embodiments Cycle-D is 5-membered cycloalkyl optionally substituted with 1, 2, or 3 substituents independently selected from R² as allowed by valence.

In certain embodiments Cycle-D is 6-membered cycloalkyl optionally substituted with 1, 2, or 3 substituents independently selected from R² as allowed by valence.

### Embodiments of Tricyclic Cereblon Ligand

In certain embodiments Tricyclic Cereblon Ligand is selected from:

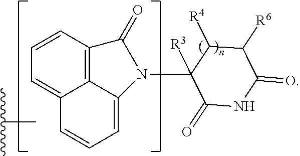

In certain embodiments Tricyclic Cereblon Ligand is selected from:

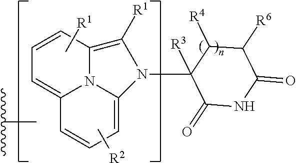

In certain embodiments Tricyclic Cereblon Ligand is selected from:

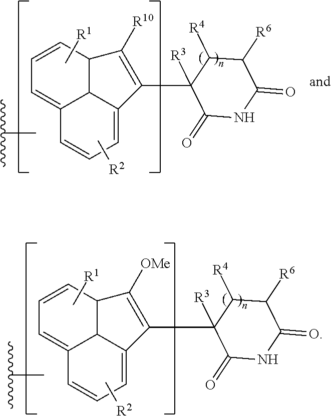

In certain embodiments Tricyclic Cereblon Ligand is selected from:

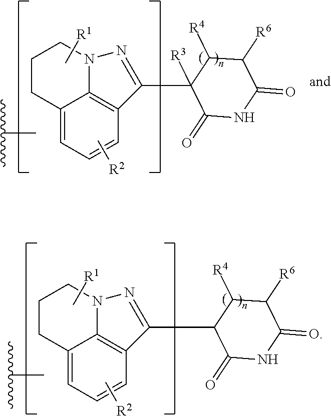

In certain embodiments Tricyclic Cereblon Ligand is selected from:

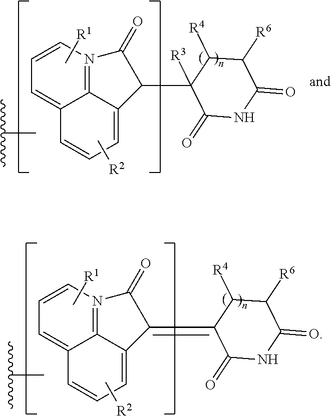

In certain embodiments Tricyclic Cereblon Ligand is selected from:

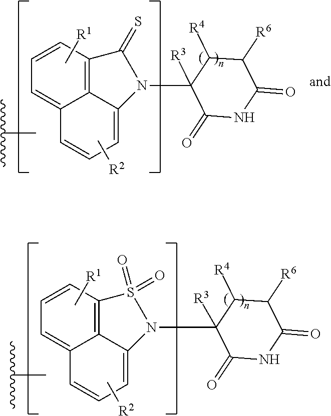

In certain embodiments Tricyclic Cereblon Ligand is selected from:

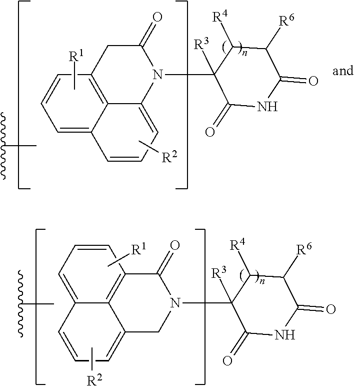

In certain embodiments Tricyclic Cereblon Ligand is selected from:

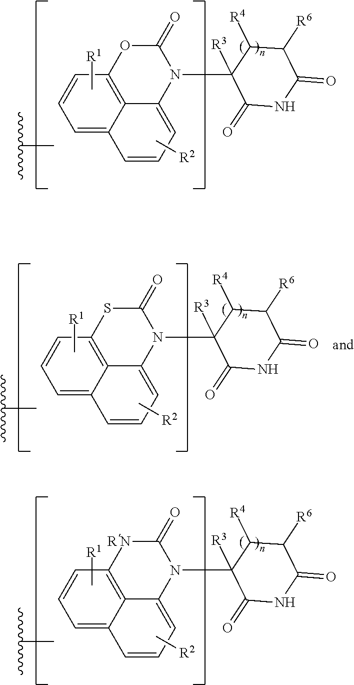

In certain embodiments Tricyclic Cereblon Ligand is selected from:

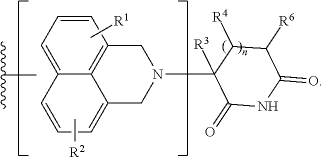

In certain embodiments Tricyclic Cereblon Ligand is selected from:

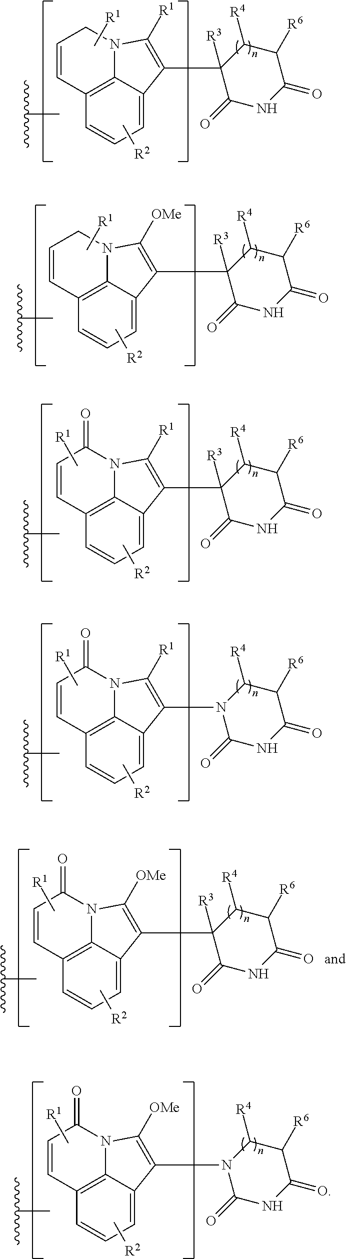

In certain embodiments Tricyclic Cereblon Ligand is selected from:

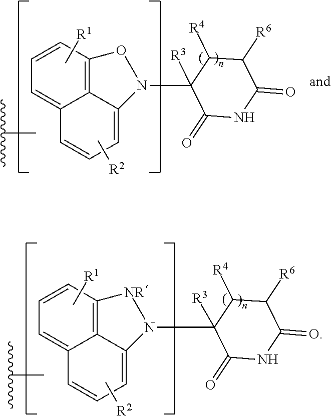

In certain embodiments Tricyclic Cereblon Ligand is selected from:

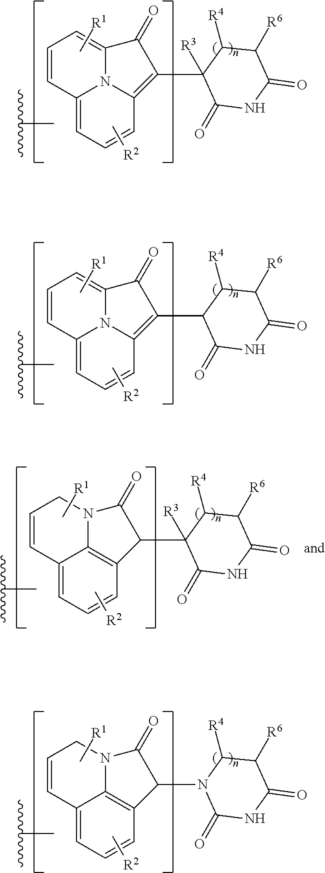

In certain embodiments Tricyclic Cereblon Ligand is selected from:

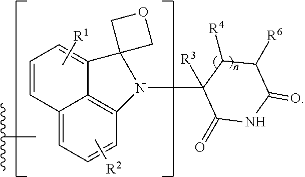

In certain embodiments Tricyclic Cereblon Ligand is selected from:

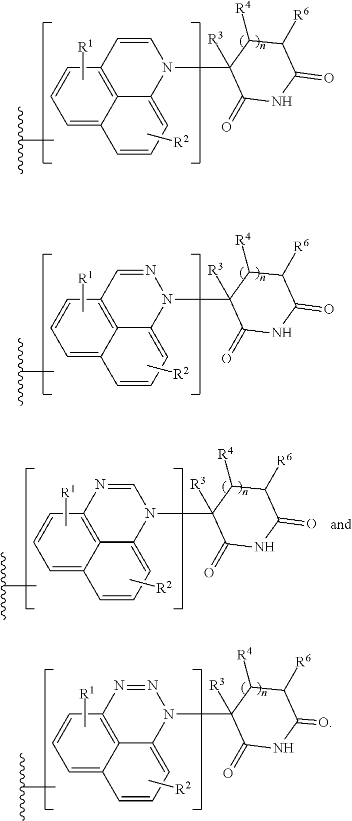

In certain embodiments Tricyclic Cereblon Ligand is selected from:

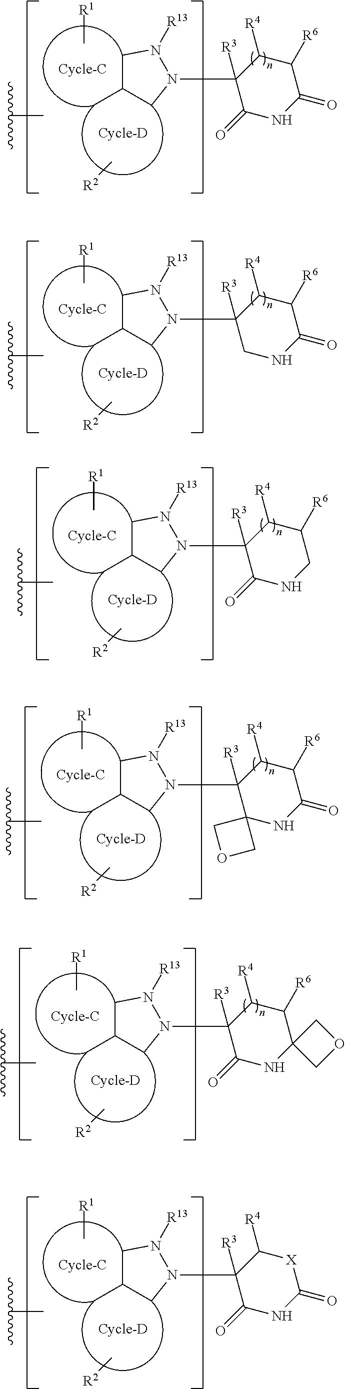

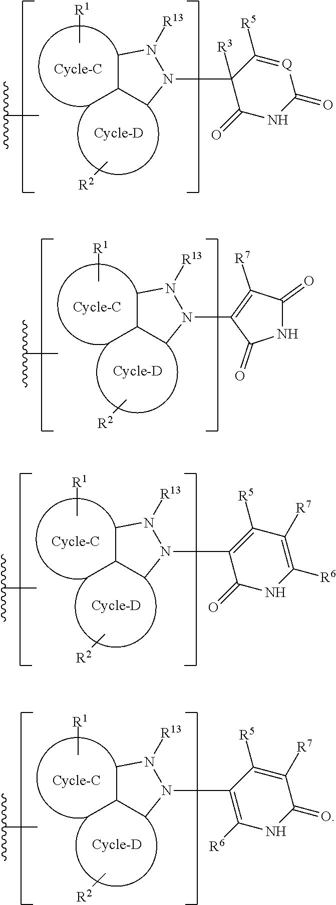

In certain embodiments Tricyclic Cereblon Ligand is selected from:

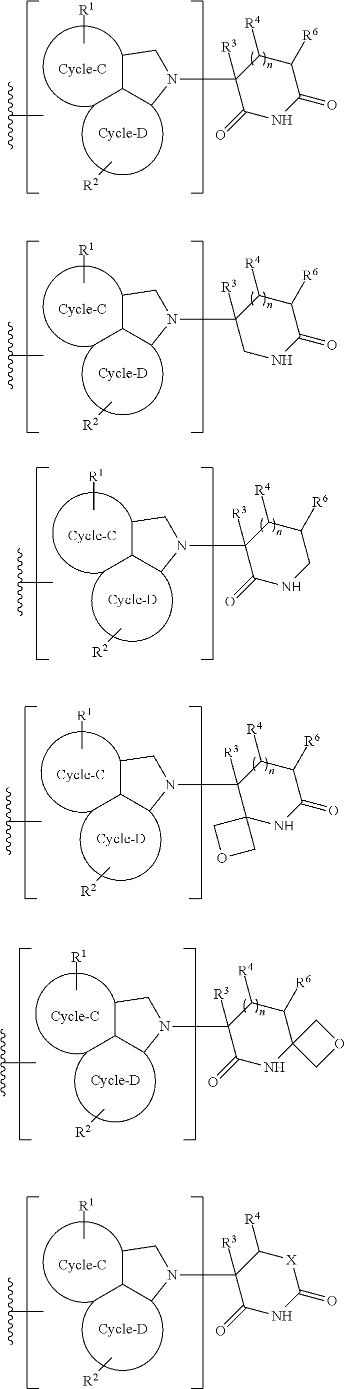

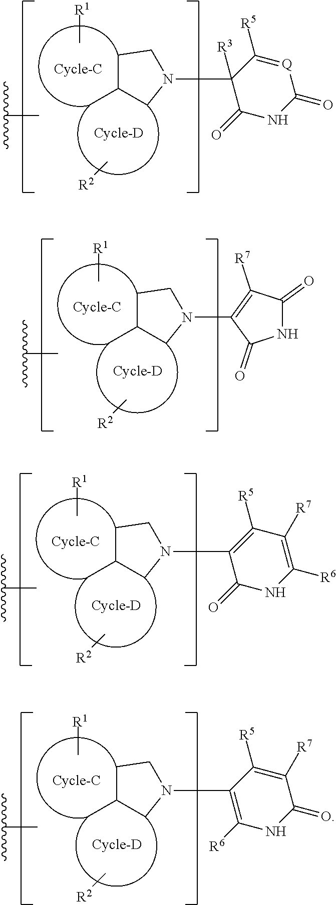

In certain embodiments Tricyclic Cereblon Ligand is selected from:

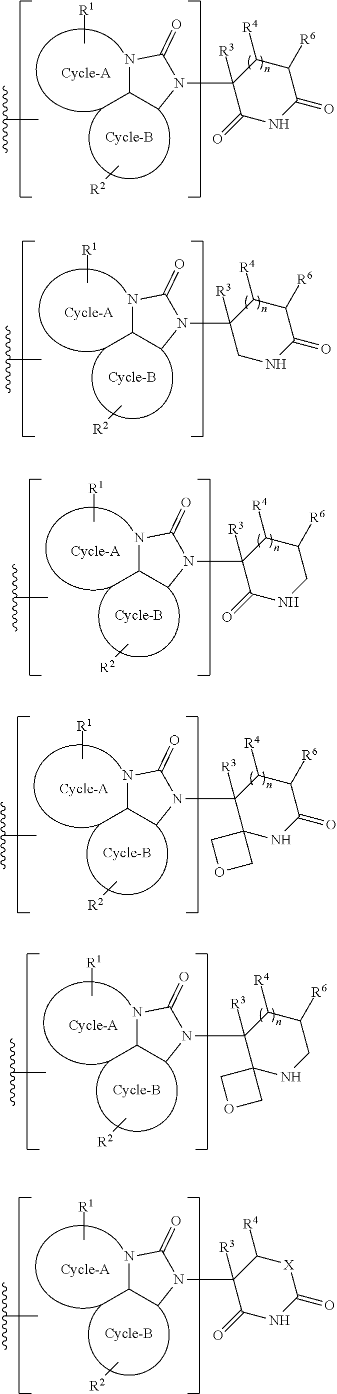

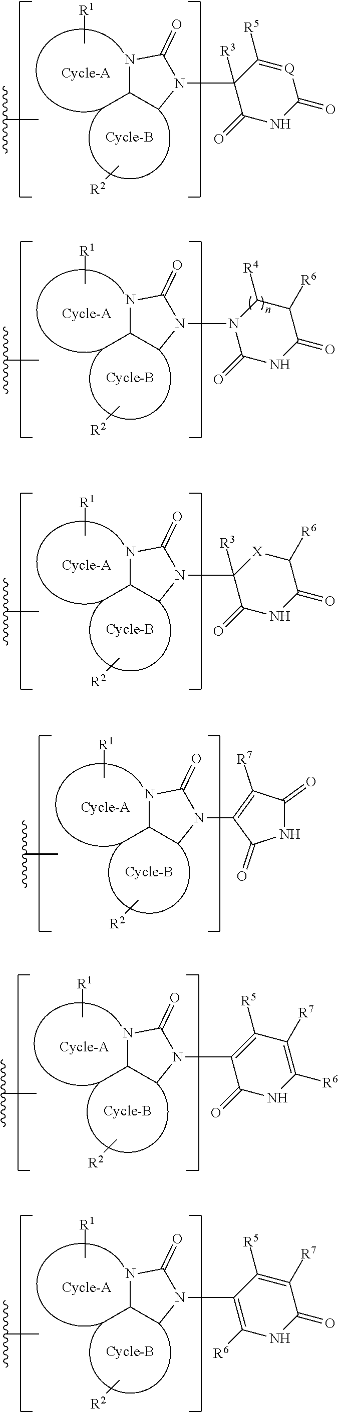

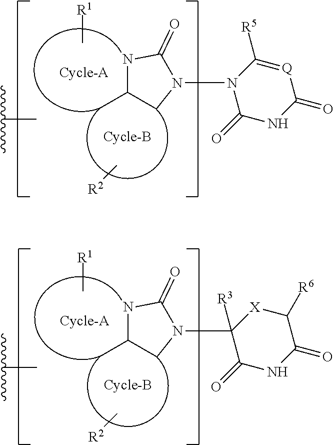

In certain embodiments Tricyclic Cereblon Ligand is selected from:

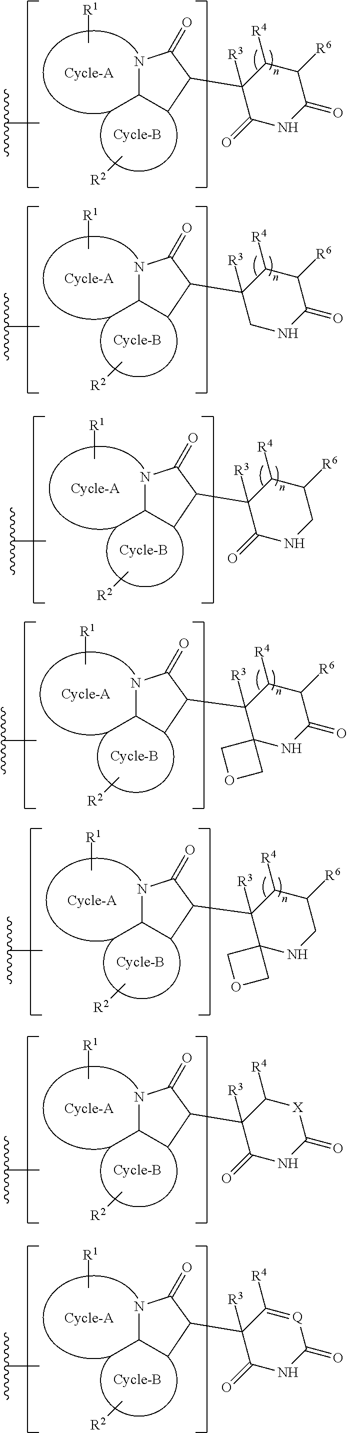

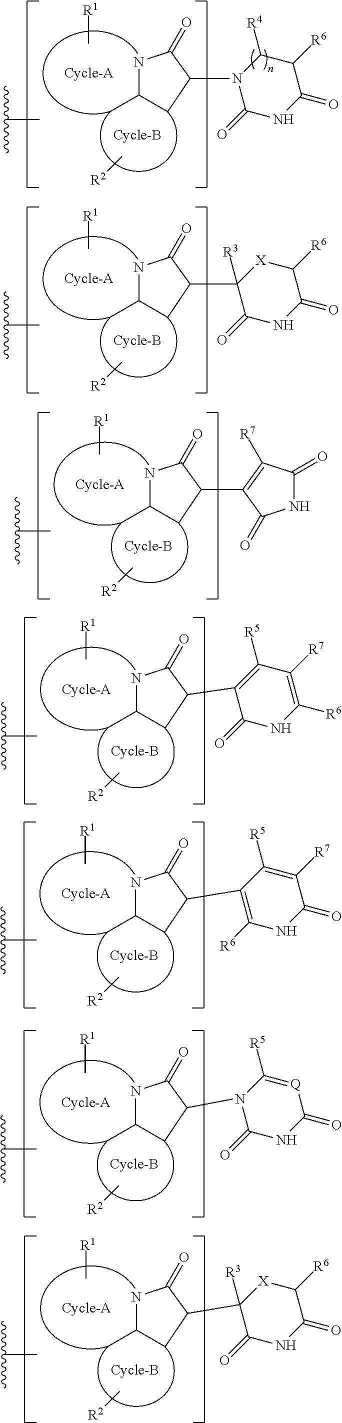

In an alternative embodiment the Cereblon Binding Ligand is:

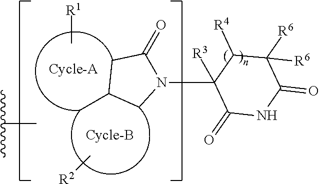

wherein each R⁶ is independently selected from selected from hydrogen, alkyl, halogen, haloalkyl, —OR¹⁰, —SR¹⁰, —S(O)R¹², —SO₂R¹², and —NR¹⁰R¹¹; or two R⁶ groups are combined together to form a 3- to 4-membered spirocycle.

In an alternative embodiment the Cereblon Binding Ligand is:

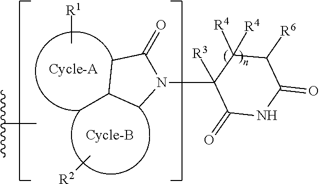

wherein each R⁴ is independently selected from selected from hydrogen, alkyl, halogen, and haloalkyl; or two R⁴ groups are combined together to form a 3- to 6-membered cycle.

In an alternative embodiment the Cereblon Binding Ligand is:

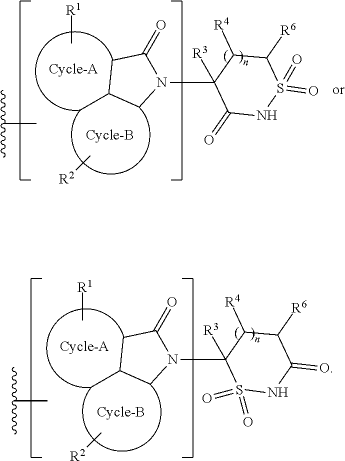

In an alternative embodiment the Cereblon Binding Ligand is:

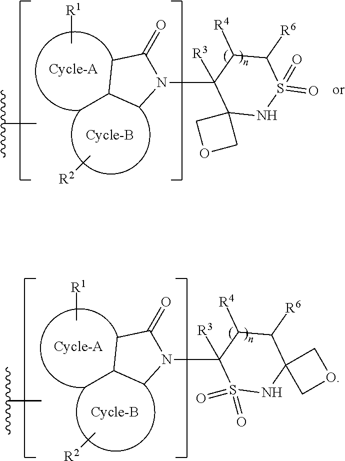

In certain embodiments the compound of the present invention is selected from:

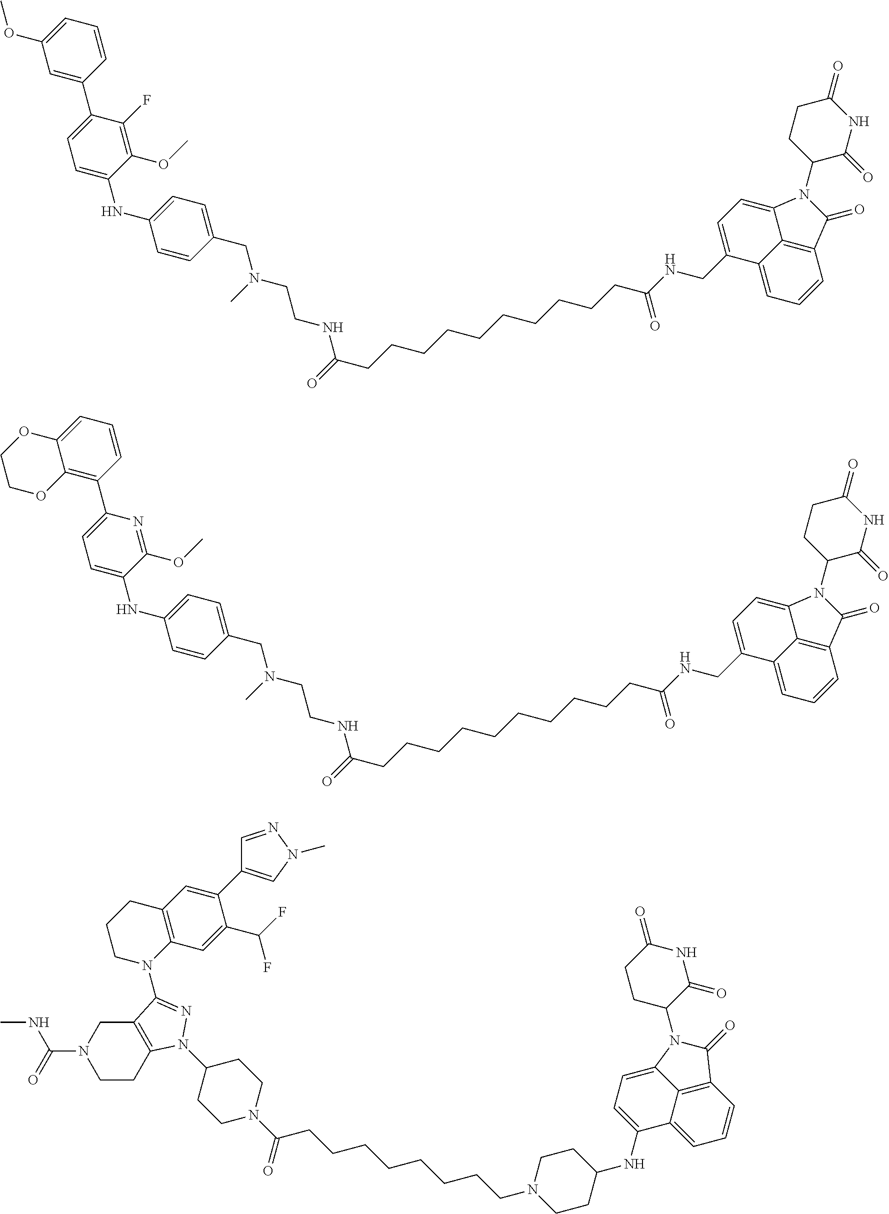 [SMILES: COc1cccc(-c2ccc(Nc3ccc(CN(C)CCNC(=O)CCCCCCCCCCC(=O)NCc4ccc5c6c(cccc46)C(=O)N5C4CCC(=O)NC4=O)cc3)c(OC)c2F)c1 ; COc1nc(-c2cccc3c2OCCO3)ccc1Nc1ccc(CN(C)CCNC(=O)CCCCCCCCCCC(=O)NCc2ccc3c4c(cccc24)C(=O)N3C2CCC(=O)NC2=O)cc1 ; CNC(=O)N1CCc2c(c(N3CCCc4cc(-c5cnn(C)c5)c(C(F)F)cc43)nn2C2CCN(C(=O)CCCCCCCCN3CCC(Nc4ccc5c6c(cccc46)C(=O)N5C4CCC(=O)NC4=O)CC3)CC2)C1]

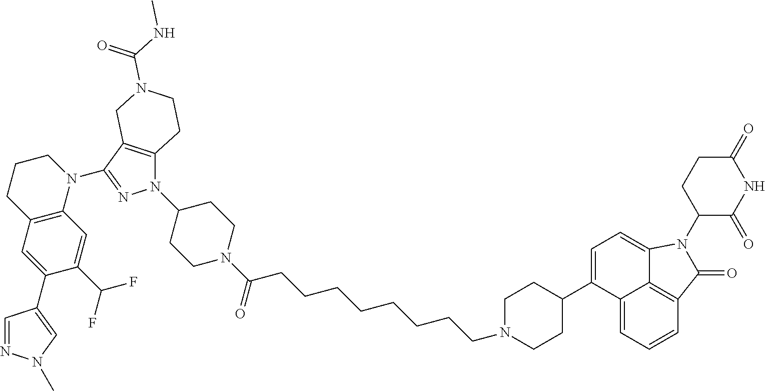 [SMILES: CNC(=O)N1CCc2c(c(N3CCCc4cc(-c5cnn(C)c5)c(C(F)F)cc43)nn2C2CCN(C(=O)CCCCCCCCN3CCC(c4ccc5c6c(cccc46)C(=O)N5C4CCC(=O)NC4=O)CC3)CC2)C1]

### Non-Limiting Isotopic Embodiments

In certain embodiments the compound is isotopically labeled. In certain embodiments at least one R group independently selected from R¹, R², R³, R⁴, R⁶, R⁷, R¹⁰, R¹¹, R¹², R¹³, R¹⁴, R¹⁵, R¹⁶, R¹⁷, R¹⁸, R²⁰, R²¹, R²², R²³, R²⁴, R²⁶, R²⁷, R⁴⁰, R⁴¹, or R⁴² is isotopically labeled with 1, 2, or more isotopes as allowed by valence. In certain embodiments the isotopic label is deuterium. In certain embodiments, at least one deuterium is placed on an atom that has a bond which is broken during metabolism of the compound in vivo, or is one, two or three atoms remote form the metabolized bond (e.g., which may be referred to as an α, β or γ, or primary, secondary or tertiary isotope effect). In another embodiment the isotopic label is ¹³C. In another embodiment the isotopic label is ¹⁸F.

### Additional Embodiments

1. In certain embodiments a compound is provided of Formula I

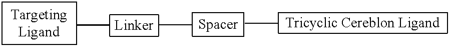

or a pharmaceutically acceptable salt, N-oxide, isotopic derivative, or prodrug thereof, optionally in a pharmaceutically acceptable carrier to form a composition;
wherein:

The Tricyclic Cereblon Ligand is selected from one of the following moieties, wherein the bracketed bond indicates that the tricyclic moiety is attached to the Spacer/Linker via a covalent bond on Cycle-A, Cycle-B, Cycle-C or Cycle-D as relevant in a manner that achieves the desired potency and catalytic degradation profile.

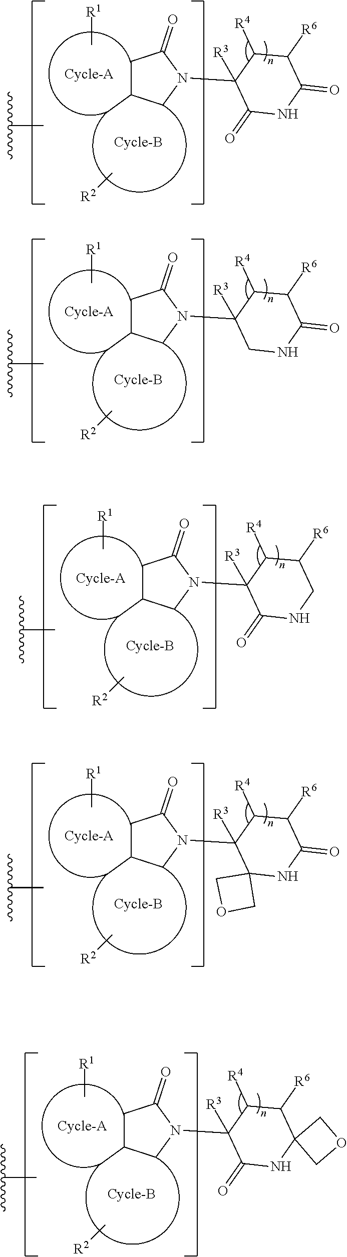

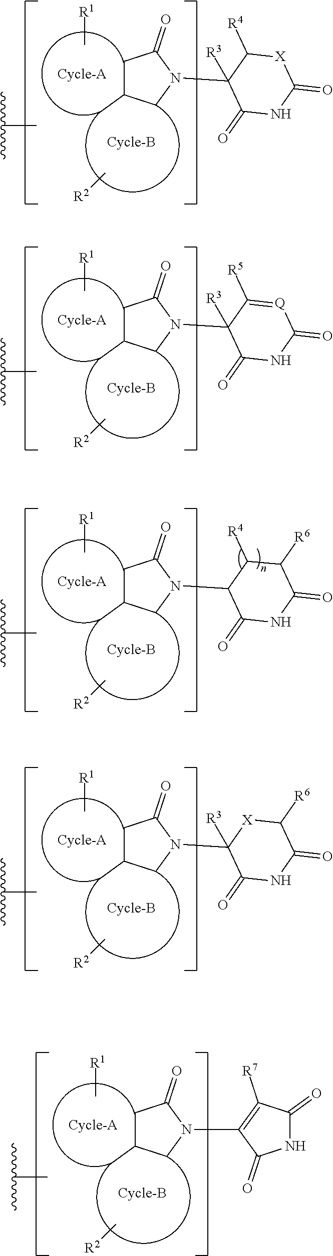

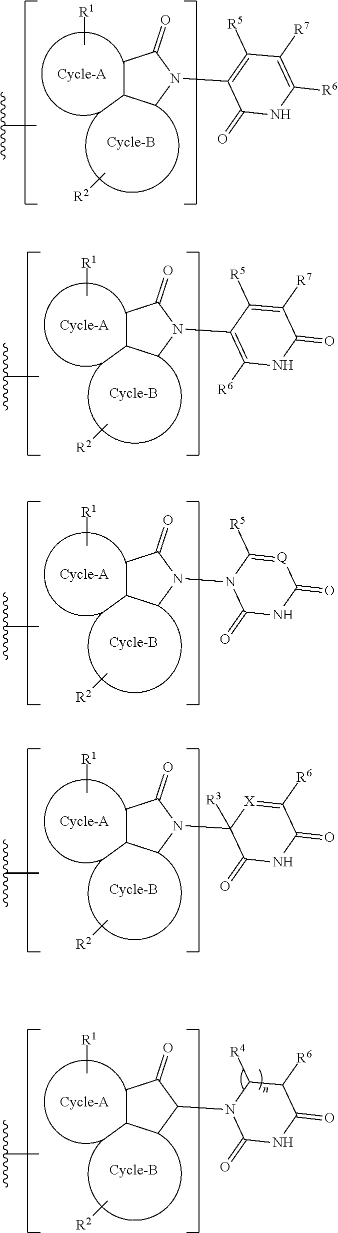

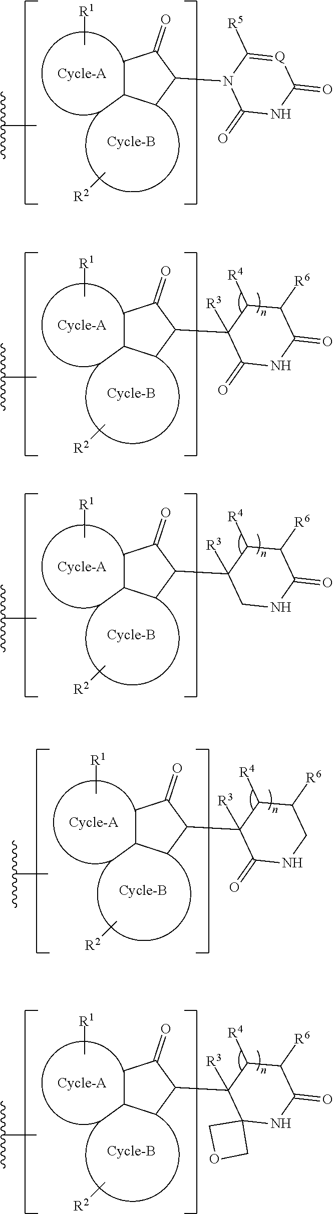

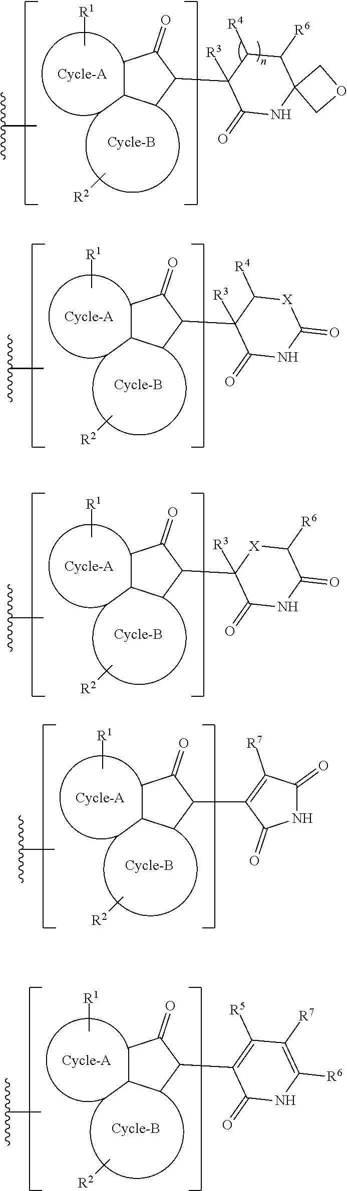

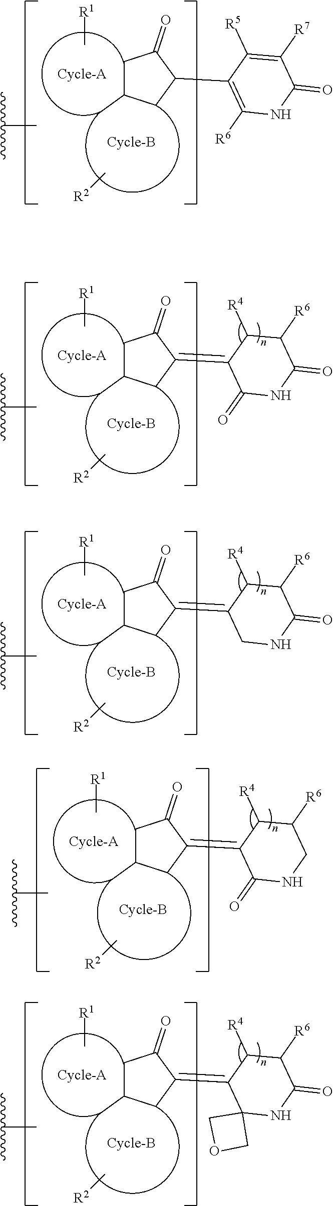

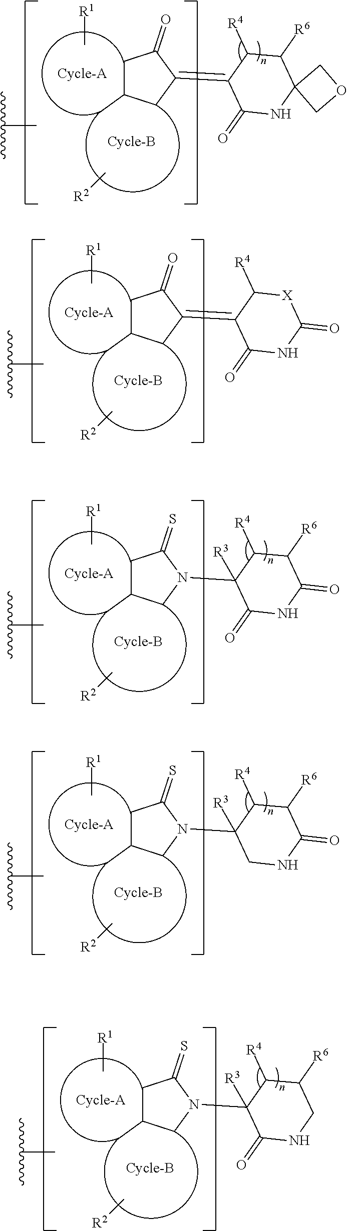

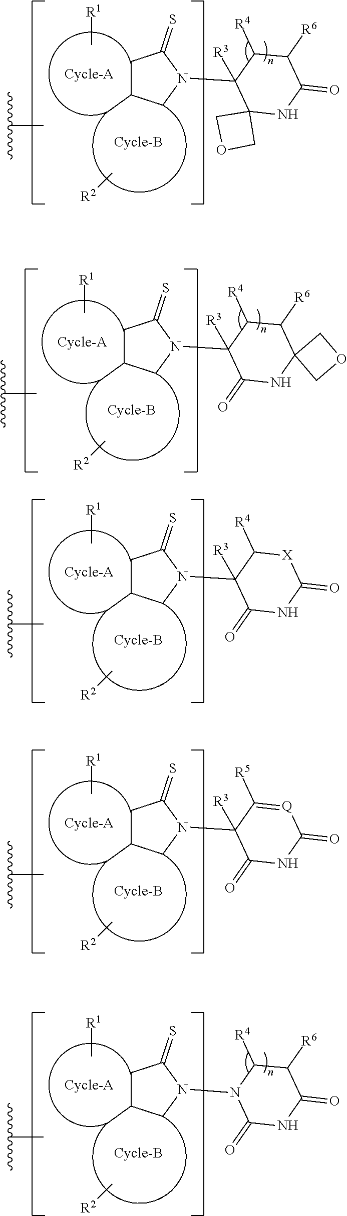

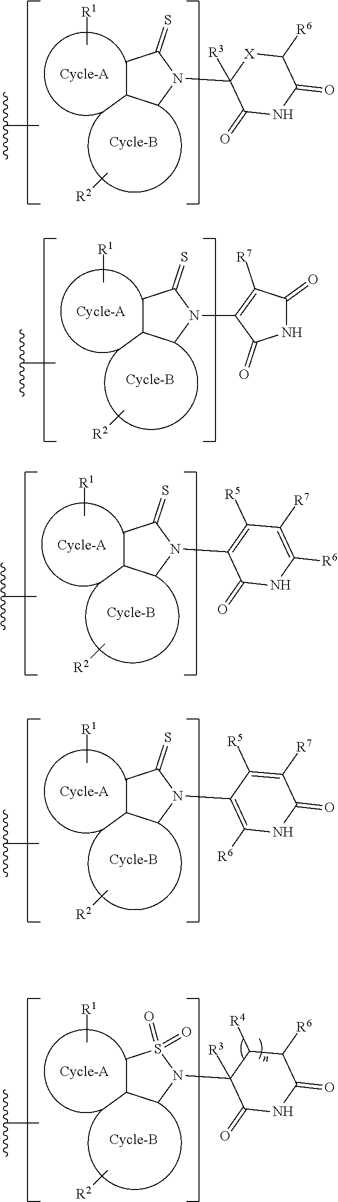

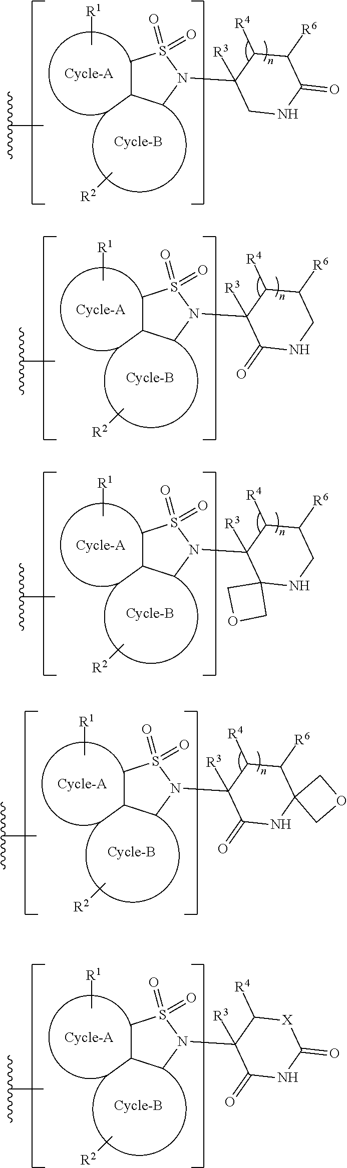

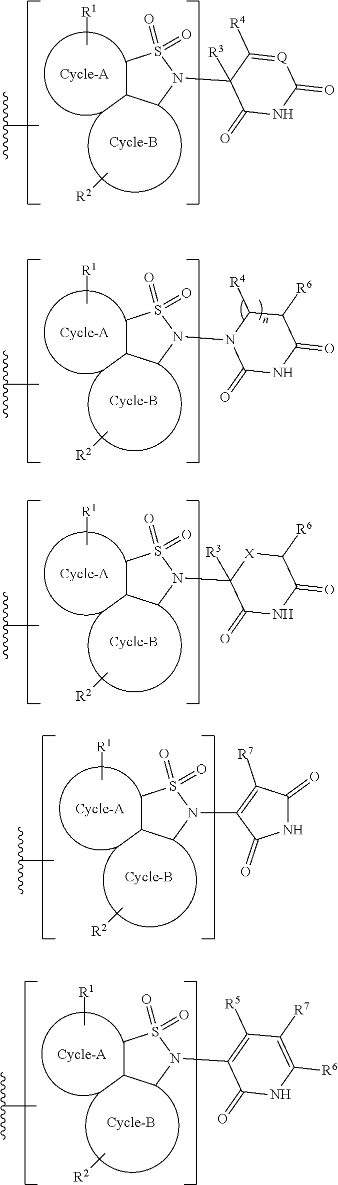

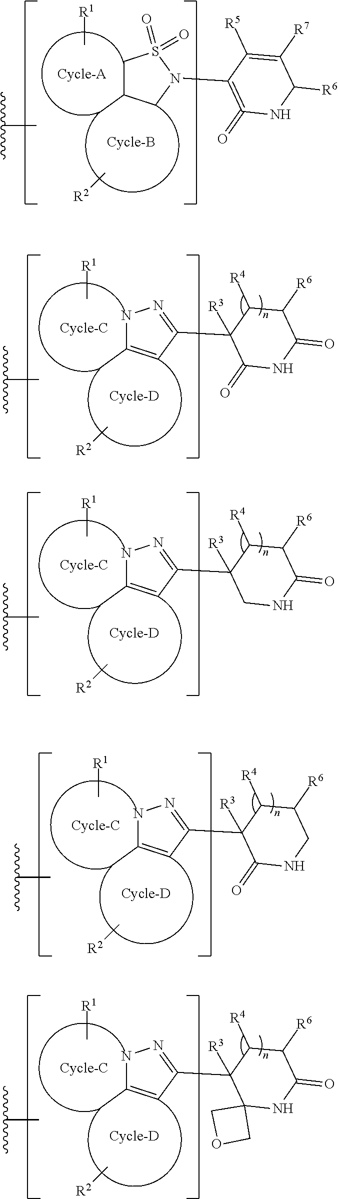

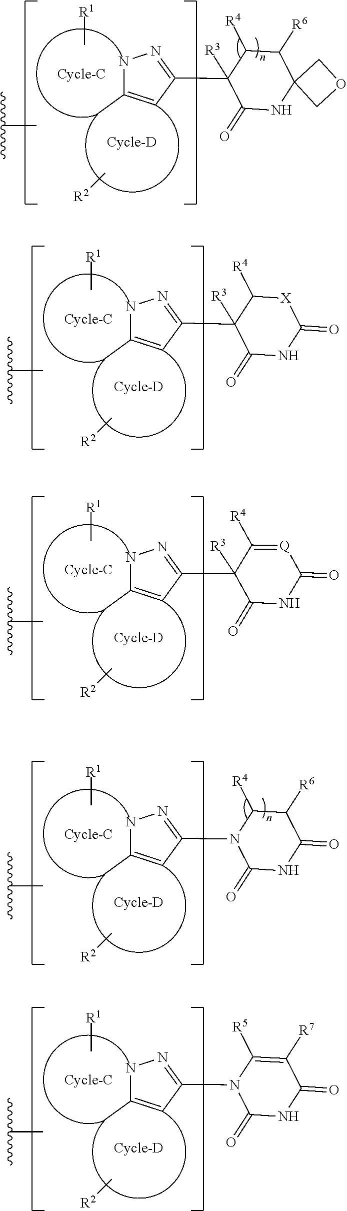

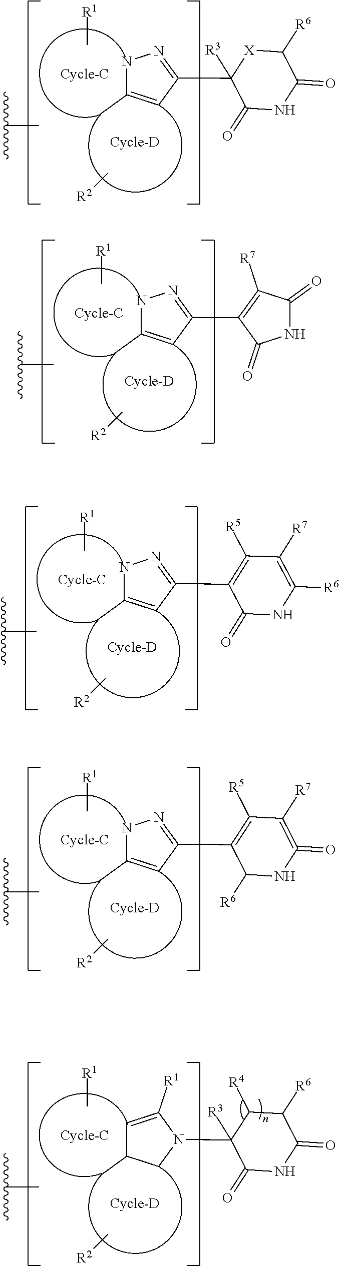

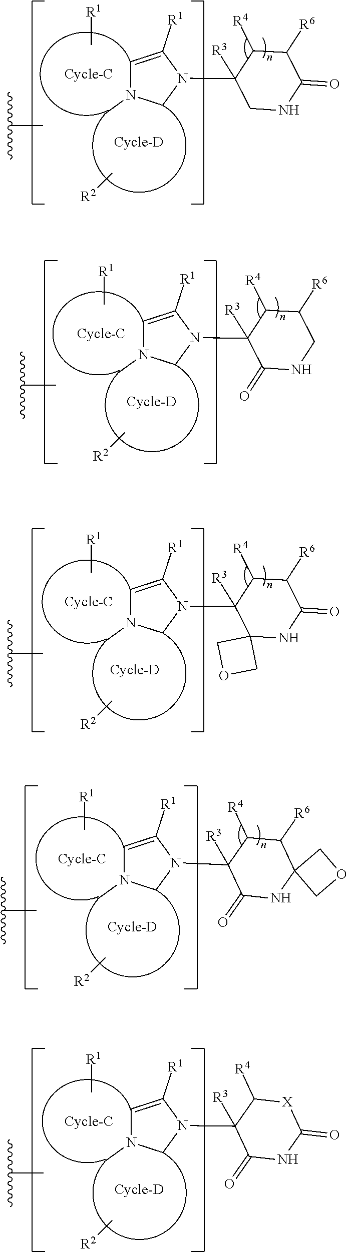

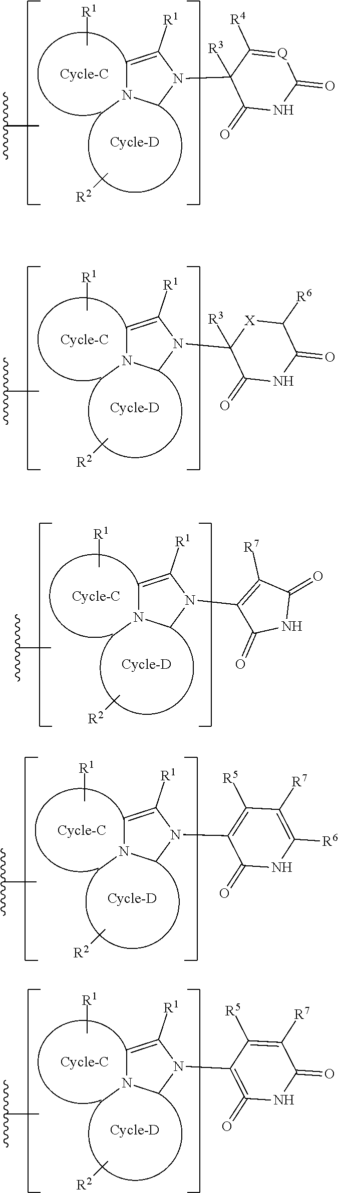

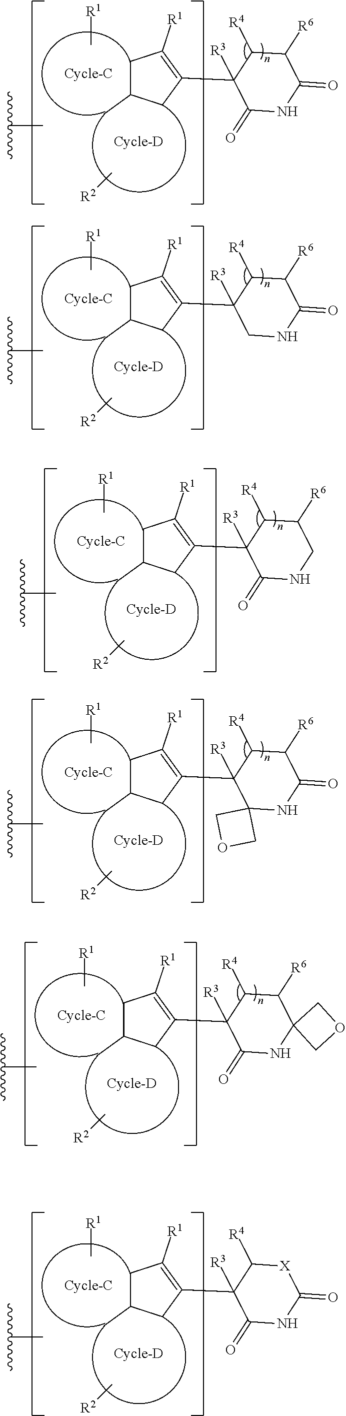

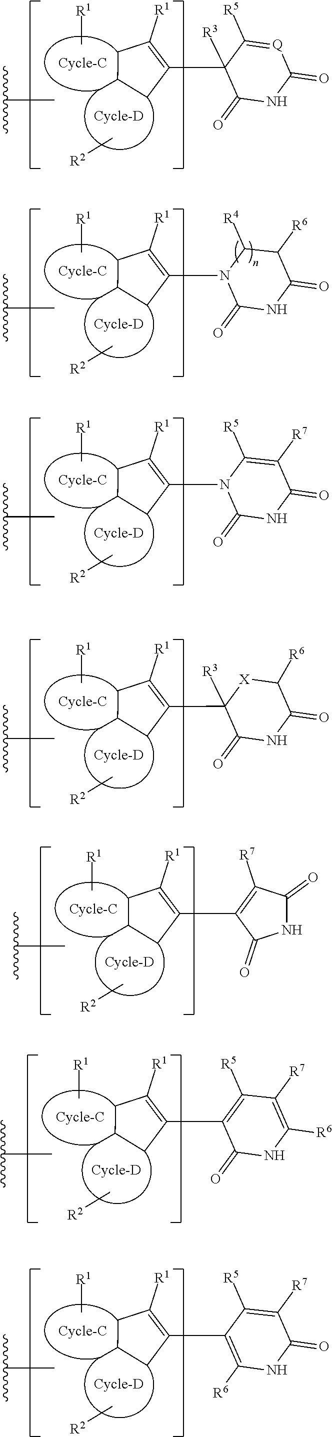

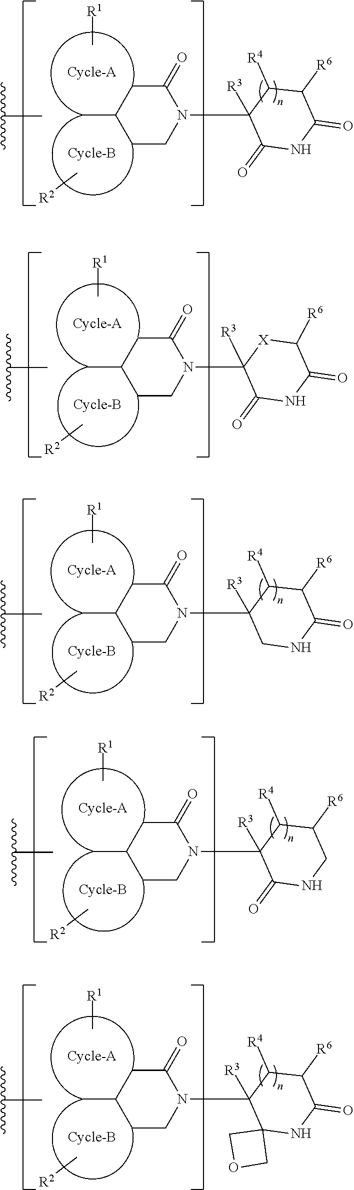

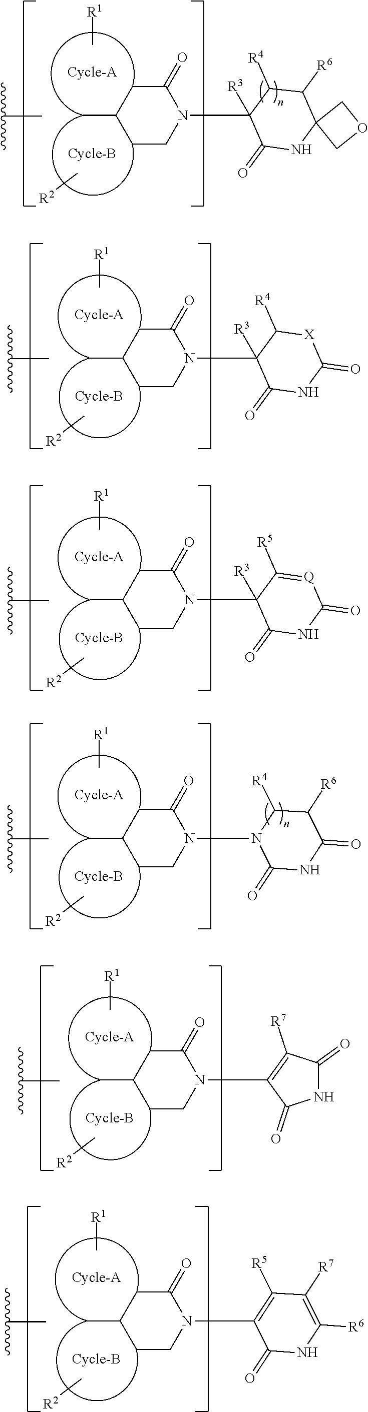

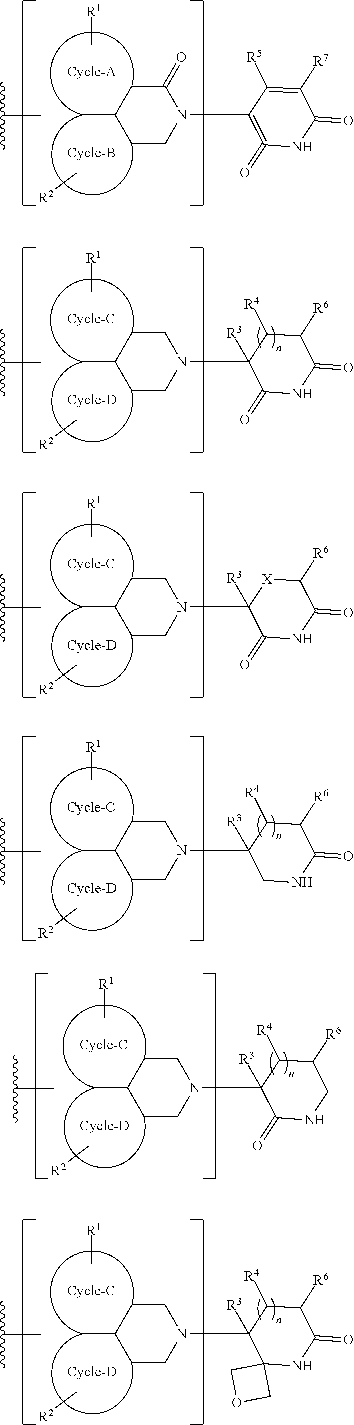

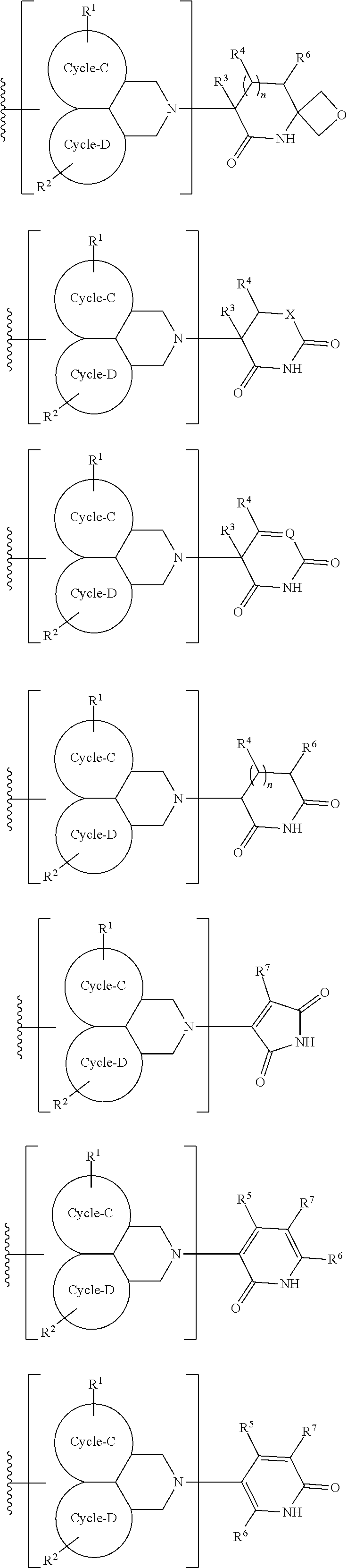

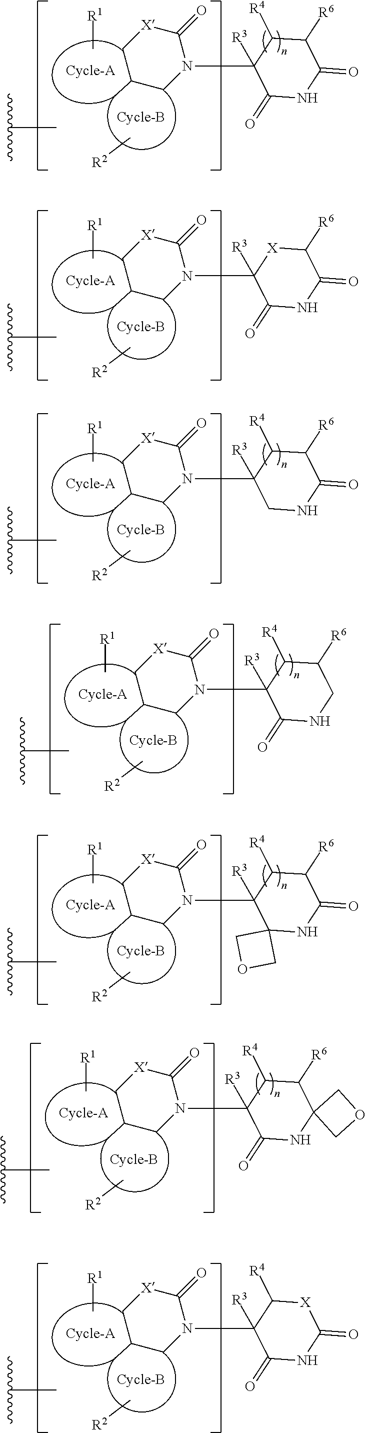

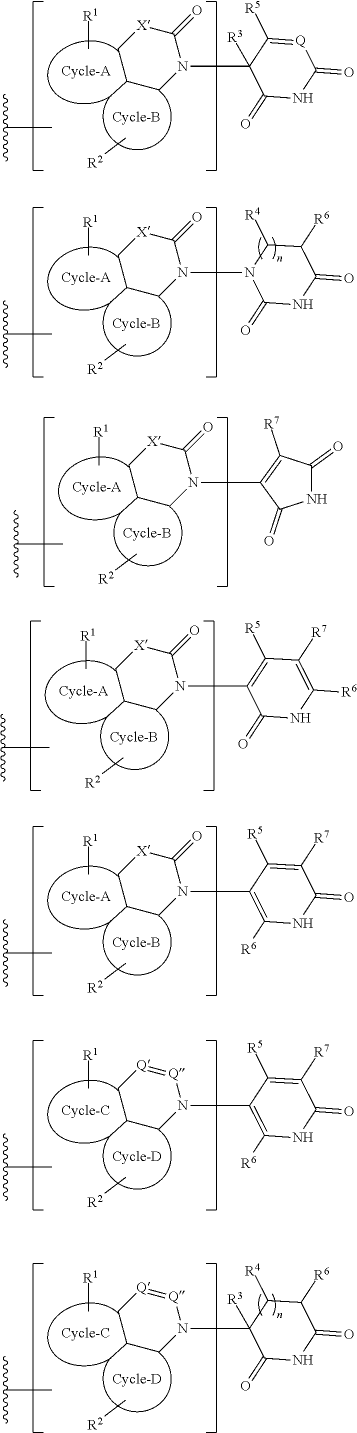

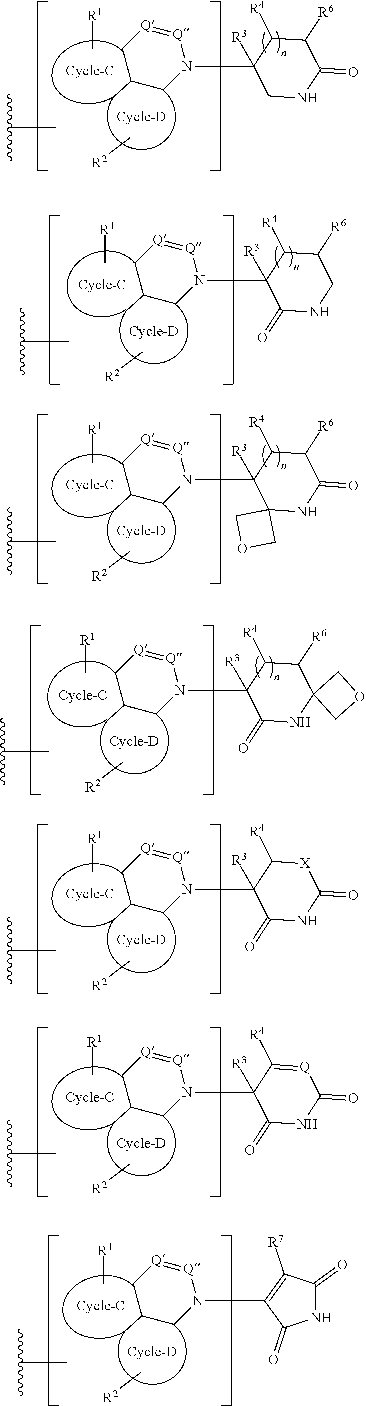

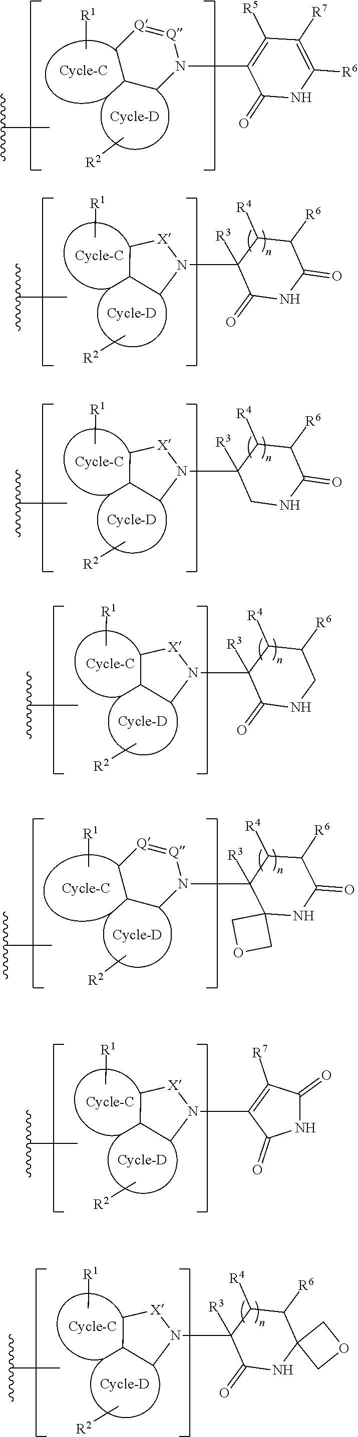

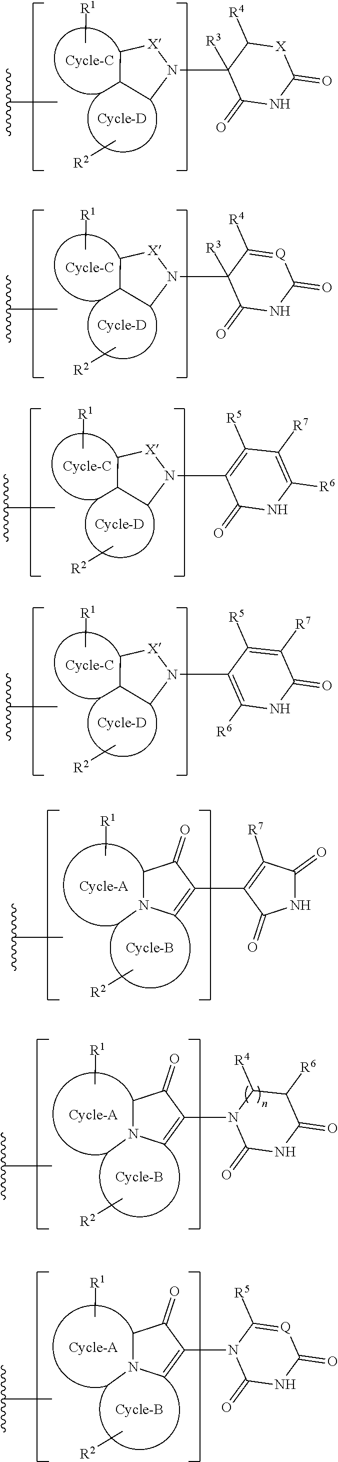

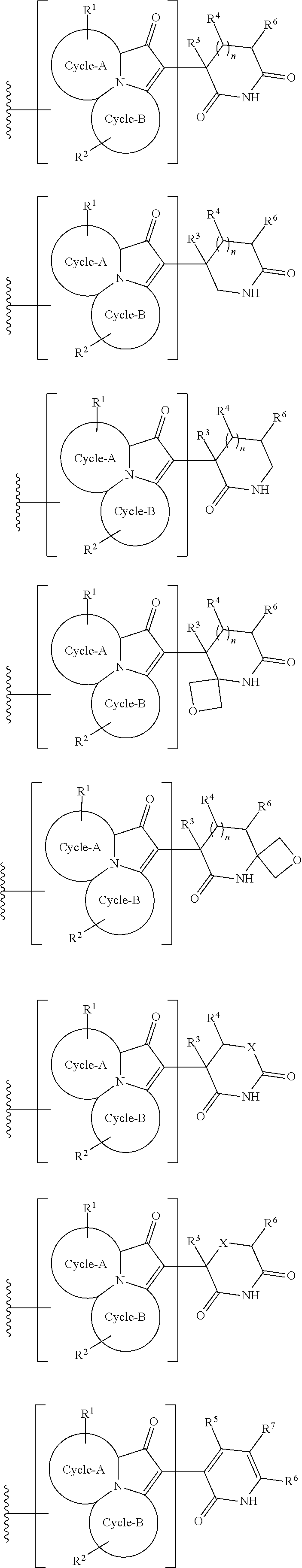

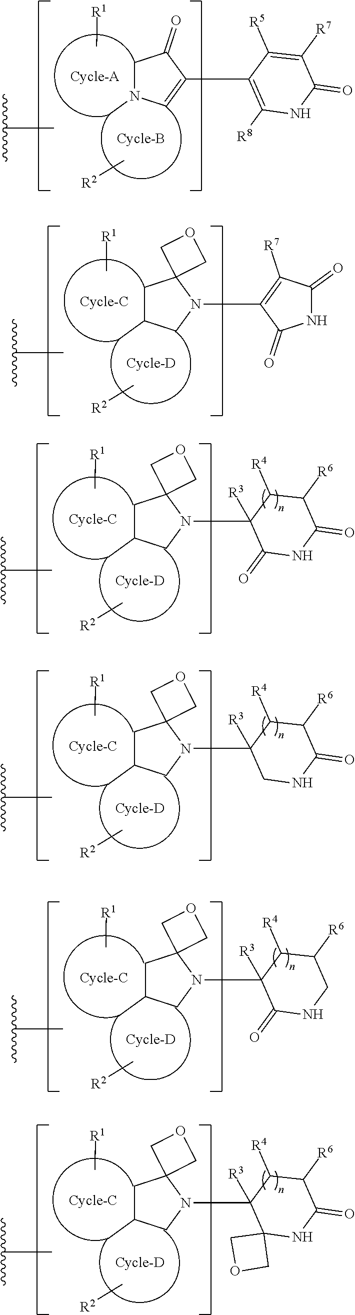

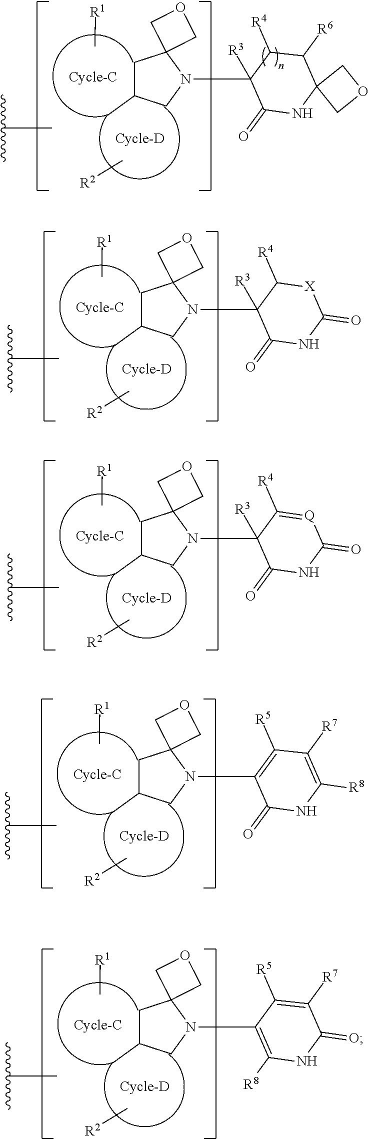

n is 0, 1, or 2;
X is NR¹⁰, NR^{6′}, O, or S;
X′ is NR¹⁰, O, CH₂, or S;
Q is CR⁷ or N;
Q′ and Q″ are independently selected from CR¹ and N.
Cycle-A is a fused ring selected from phenyl, 5- or 6-membered heteroaryl, 5- to 8-membered heterocycle, 5- to 8-membered cycloalkyl, or 5- to 8-membered cycloalkenyl, wherein Cycle-A is optionally substituted with 1, 2, or 3 substituents independently selected from R¹ as allowed by valence.
Cycle-B is a fused ring selected from phenyl, 5- or 6-membered heteroaryl, 5- to 8-membered heterocycle, 5- to 8-membered cycloalkyl, or 5- to 8-membered cycloalkenyl, wherein Cycle-B is optionally substituted with 1, 2, or 3 substituents independently selected from R² as allowed by valence.

In certain embodiments Cycle-A is a fused ring selected from phenyl, 5- or 6-membered heteroaryl, 5- to 6-membered heterocycle, 5- to 6-membered cycloalkyl, or 5- to 6-membered cycloalkenyl, wherein Cycle-A is optionally substituted with 1, 2, or 3 substituents independently selected from R¹ as allowed by valence.

In certain embodiments Cycle-B is a fused ring selected from phenyl, 5- or 6-membered heteroaryl, 5- to 6-membered heterocycle, 5- to 6-membered cycloalkyl, or 5- to 6-membered cycloalkenyl, wherein Cycle-B is optionally substituted with 1, 2, or 3 substituents independently selected from R² as allowed by valence.
Cycle-C is a fused ring selected from phenyl, 5- or 6-membered heteroaryl, 5- to 6-membered heterocycle, 5- to 6-membered cycloalkyl, or 5- to 6-membered cycloalkenyl, wherein each Cycle-C is optionally substituted with 1, 2, or 3 substituents independently selected from R¹ as allowed by valence.
Cycle-D is a fused ring selected from phenyl, 5- or 6-membered heteroaryl, 5 to 6-membered heterocycle, 5- to 6-membered cycloalkyl, or 5- to 6-membered cycloalkenyl, wherein each Cycle-D is optionally substituted with 1, 2, or 3 substituents independently selected from R² as allowed by valence.
R¹ and R² are independently at each instance selected from hydrogen, alkyl, halogen, haloalkyl, —OR¹⁰, —SR¹⁰, —S(O)R¹², —SO₂R¹², —NR¹⁰R¹¹, cyano, nitro, heteroaryl, aryl, and heterocycle; or alternatively, if allowed by valence and stability, R¹ or R² may be a divalent moiety such as ═O, ═S, or ═NR⁴¹; and wherein an R¹ group may optionally be combined with another R¹ group or an R² group to form a fused cycle or bicycle which may bridge Cycle-A and Cycle-B or Cycle-C and Cycle-D, as appropriate and desired.
R³ is hydrogen, alkyl, halogen, or haloalkyl;
or R³ and R⁶ are combined to form a 1 or 2 carbon attachment, for example when R³ and R⁶ form a 1 carbon attachment

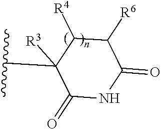

is

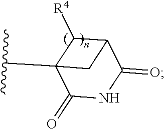

or R³ and R⁴ are combined to form a 1, 2, 3, or 4 carbon attachment, for example when R³ and R⁴ form a 1 carbon attachment

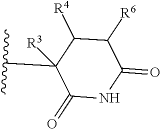

is

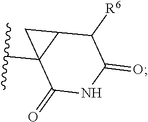

or R³ and an R⁴ group adjacent to R³ are combined to form a double bond.
R⁴ and R⁵ are independently selected from hydrogen, alkyl, halogen, and haloalkyl;
R⁶ and R⁷ are independently selected from hydrogen, alkyl, halogen, haloalkyl, —OR¹⁰, —SR¹⁰, —S(O)R¹², —SO₂R¹², and —NR¹⁰R¹¹, R^{6′} is hydrogen, alkyl, or haloalkyl;
or R³ and R^{6′} are combined to form a 1 or 2 carbon attachment.
R¹⁰ and R¹¹ are independently selected from hydrogen, alkyl, haloalkyl, heterocycle, aryl, heteroaryl, —C(O)R¹², —S(O)R¹², and —SO₂R¹²;
each R¹² is independently selected from hydrogen, alkyl, haloalkyl, heterocycle, aryl, heteroaryl, —NR¹³R¹⁴, and OR¹³;
and each instance of R¹³ and R¹⁴ is independently selected from hydrogen, alkyl, and haloalkyl.

Spacer is a bivalent connecting moiety which may be of the structure:

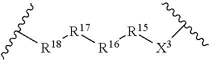

X³ is a bivalent moiety selected from bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂— and —S—; or can be arylalkyl, heterocyclealkyl or heteroarylalkyl (in either direction), each of which heterocycle, aryl, heteroaryl, and bicycle may be substituted with 1, 2, 3, or 4 substituents independently selected from R⁴⁰;
R¹⁵, R¹⁶, R¹⁷, and R¹⁸ are independently at each occurrence selected from the group consisting of a bond, alkyl (which in certain embodiments is a carbocycle), —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, arylalkyl, heterocyclealkyl, and heteroarylalkyl; each of which is optionally substituted with 1, 2, 3, or 4 substituents independently selected from R⁴⁰;
wherein X³ and R¹⁵⁻¹⁸ together are a stable moiety covalently connecting the Tricyclic Cereblon Ligand to the Linker, and wherein in certain embodiments Spacer is a covalent bond;
R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, oxo, and cycloalkyl;
R⁴¹ is aliphatic (including alkyl), aryl, heteroaryl, or hydrogen;
Targeting Ligand is a moiety that binds to a Target Protein and is covalently linked to the Tricyclic Cereblon Ligand through the Linker-Spacer;
Target Protein is a selected protein that causes or contributes to the disease to be treated in vivo;
Linker is a bivalent linking group, for example a bivalent linking group of Formula LI.
2. The compound of embodiment 1, wherein Cycle-A is a fused ring selected from phenyl, 5- or 6-membered heteroaryl, 5- to 6-membered heterocycle, 5- to 6-membered cycloalkyl, or 5- to 6-membered cycloalkenyl, wherein Cycle-A is optionally substituted with 1, 2, or 3 substituents independently selected from R¹ as allowed by valence.
3. The compound of embodiment 1, wherein Cycle-A is phenyl optionally substituted with 1, 2, or 3 substituents independently selected from R¹ as allowed by valence.
4. The compound of embodiment 1, wherein Cycle-A is 5-membered heteroaryl optionally substituted with 1, 2, or 3 substituents independently selected from R¹ as allowed by valence.
5. The compound of embodiment 1, wherein Cycle-A is 6-membered heteroaryl optionally substituted with 1, 2, or 3 substituents independently selected from R¹ as allowed by valence.
6. The compound of embodiment 1, wherein Cycle-A is 5-membered heterocycle optionally substituted with 1, 2, or 3 substituents independently selected from R¹ as allowed by valence.
7. The compound of embodiment 1, wherein Cycle-A is 6-membered heterocycle optionally substituted with 1, 2, or 3 substituents independently selected from R¹ as allowed by valence.
8. The compound of embodiment 1, wherein Cycle-A is 7-membered heterocycle optionally substituted with 1, 2, or 3 substituents independently selected from R¹ as allowed by valence.
9. The compound of embodiment 1, wherein Cycle-A is 8-membered heterocycle optionally substituted with 1, 2, or 3 substituents independently selected from R¹ as allowed by valence.
10. The compound of embodiment 1, wherein Cycle-A is 5-membered cycloalkyl optionally substituted with 1, 2, or 3 substituents independently selected from R¹ as allowed by valence.
11. The compound of embodiment 1, wherein Cycle-A is 6-membered cycloalkyl optionally substituted with 1, 2, or 3 substituents independently selected from R¹ as allowed by valence.
12. The compound of embodiment 1, wherein Cycle-A is 7-membered cycloalkyl optionally substituted with 1, 2, or 3 substituents independently selected from R¹ as allowed by valence.
13. The compound of embodiment 1, wherein Cycle-A is 8-membered cycloalkyl optionally substituted with 1, 2, or 3 substituents independently selected from R¹ as allowed by valence.
14. The compound of any one of embodiments 1-13, wherein Cycle-B is phenyl optionally substituted with 1, 2, or 3 substituents independently selected from R² as allowed by valence.
15. The compound of any one of embodiments 1-13, Cycle-B is 5-membered heteroaryl optionally substituted with 1, 2, or 3 substituents independently selected from R² as allowed by valence.
16. The compound of any one of embodiments 1-13, Cycle-B is 6-membered heteroaryl optionally substituted with 1, 2, or 3 substituents independently selected from R² as allowed by valence.
17. The compound of any one of embodiments 1-13, Cycle-B is 5-membered heterocycle optionally substituted with 1, 2, or 3 substituents independently selected from R² as allowed by valence.
18. The compound of any one of embodiments 1-13, Cycle-B is 6-membered heterocycle optionally substituted with 1, 2, or 3 substituents independently selected from R² as allowed by valence.
19. The compound of any one of embodiments 1-13, Cycle-B is 7-membered heterocycle optionally substituted with 1, 2, or 3 substituents independently selected from R² as allowed by valence.
20. The compound of any one of embodiments 1-13, Cycle-B is 8-membered heterocycle optionally substituted with 1, 2, or 3 substituents independently selected from R² as allowed by valence.
21. The compound of any one of embodiments 1-13, Cycle-B is 5-membered cycloalkyl optionally substituted with 1, 2, or 3 substituents independently selected from R² as allowed by valence.
22. The compound of any one of embodiments 1-13, Cycle-B is 6-membered cycloalkyl optionally substituted with 1, 2, or 3 substituents independently selected from R² as allowed by valence.
23. The compound of any one of embodiments 1-13, Cycle-B is 7-membered cycloalkyl optionally substituted with 1, 2, or 3 substituents independently selected from R² as allowed by valence.
24. The compound of any one of embodiments 1-13, Cycle-B is 8-membered cycloalkyl optionally substituted with 1, 2, or 3 substituents independently selected from R² as allowed by valence.
25. The compound of any one of embodiments 1-13, wherein Cycle-B is a fused ring selected from phenyl, 5- or 6-membered heteroaryl, 5- to 6-membered heterocycle, 5- to 6-membered cycloalkyl, or 5- to 6-membered cycloalkenyl, wherein Cycle-B is optionally substituted with 1, 2, or 3 substituents independently selected from R² as allowed by valence.
26. The compound of any one of embodiments 1-25, wherein R⁵ is hydrogen.
27. The compound of any one of embodiments 1-25, wherein R⁵ is alkyl.
28. The compound of any one of embodiments 1-25, wherein R⁵ is halogen.
29. The compound of any one of embodiments 1-25, wherein R⁵ is haloalkyl.
30. The compound of any one of embodiments 1-29, wherein R⁷ is hydrogen.
31. The compound of any one of embodiments 1-29, wherein R⁷ is halogen, haloalkyl, or alkyl.
32. The compound of any one of embodiments 1-29, wherein R⁷ is —OR¹⁰, —SR¹⁰, or —NR¹⁰R¹¹.
33. The compound of any one of embodiments 1-29, wherein R⁷ is —S(O)R¹², —SO₂R¹².
34. The compound of any one of embodiments 1-33, wherein Tricyclic Cereblon Ligand is selected from:

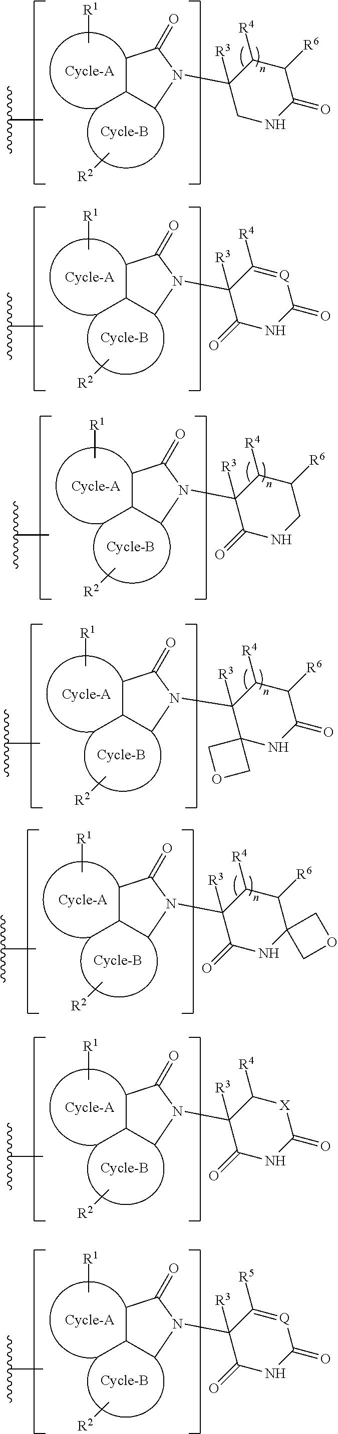

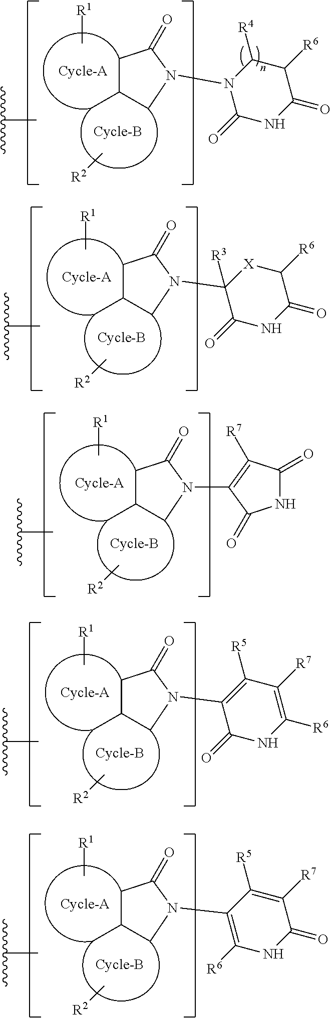

35. The compound of any one of embodiments 1-25, wherein Tricyclic Cereblon Ligand is selected from:

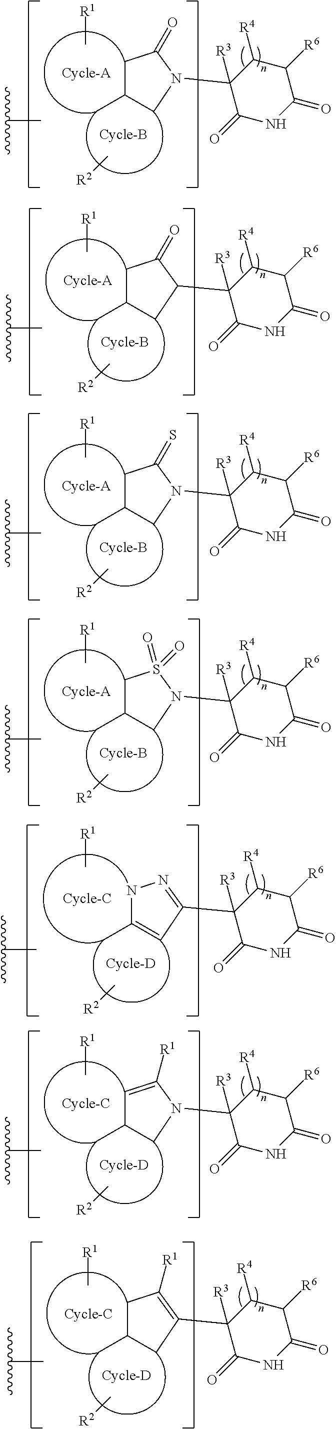

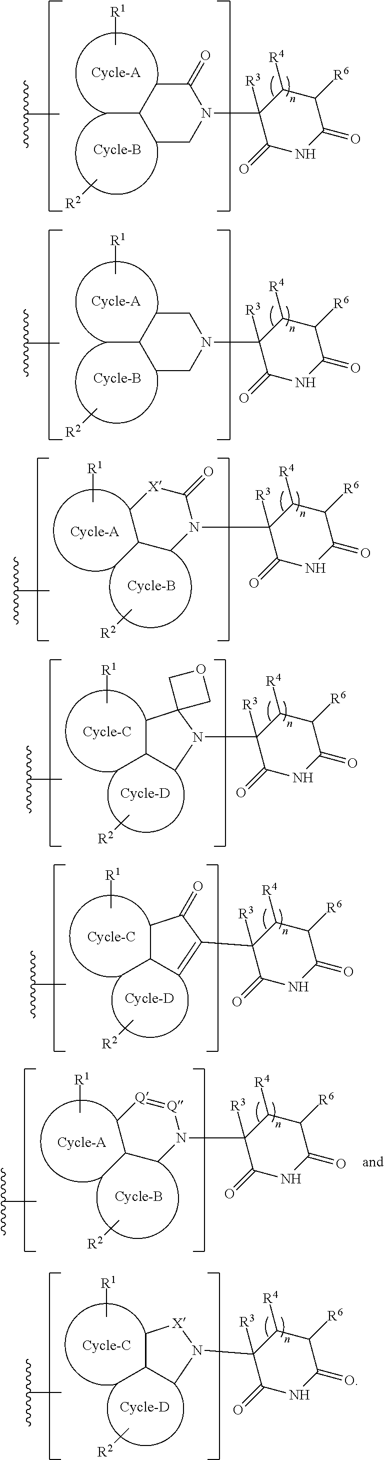

36. The compound of any one of embodiments 1-25, wherein Tricyclic Cereblon Ligand is:

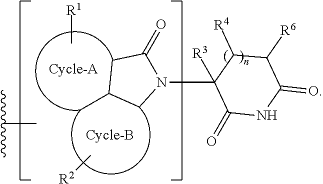

37. The compound of any one of embodiments 1-25, wherein the compound is selected from:

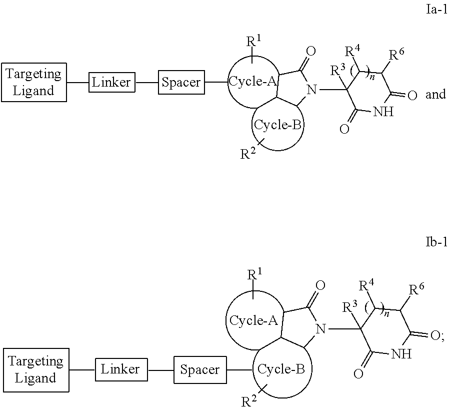

or a pharmaceutically acceptable salt thereof.
38. The compound of any one of embodiments 1-37, wherein there is 4 R² substituent.
39. The compound of any one of embodiments 1-37, wherein there is 3 R² substituent.
40. The compound of any one of embodiments 1-37, wherein there is 2 R² substituent.
41. The compound of any one of embodiments 1-37, wherein there is 1 R² substituent.
42. The compound of any one of embodiments 1-41, wherein R² is selected from alkyl, halogen, and haloalkyl.
43. The compound of any one of embodiments 1-41, wherein R² is selected from —OR¹⁰, —SR¹⁰, —S(O)R¹², —SO₂R¹², —NR¹⁰R¹¹.
44. The compound of any one of embodiments 1-41, wherein R² is selected from alkyl, halogen, and haloalkyl.
45. The compound of any one of embodiments 1-41, wherein R² is selected from, heteroaryl, aryl, and heterocycle.
46. The compound of any one of embodiments 1-40, wherein two R² substituents are combined to form a fused phenyl ring.
47. The compound of any one of embodiments 1-41, wherein at least one R² is alkyl.
48. The compound of any one of embodiments 1-41, wherein at least one R² is halogen.
49. The compound of embodiment 37, wherein the compound is selected from:

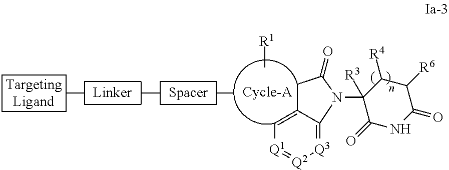

or a pharmaceutically acceptable salt thereof;
wherein Q¹, Q², and Q³ are independently selected from CH, CR¹, and N; and all other variables are as defined herein.
50. The compound of embodiment 37, wherein the compound is selected from:

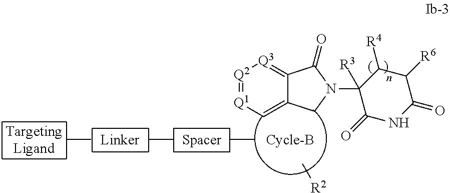

or a pharmaceutically acceptable salt thereof;
wherein Q¹, Q², and Q³ are independently selected from CH, CR¹, and N; and all other variables are as defined herein.
51. The compound of embodiment 1, wherein the compound is selected from:

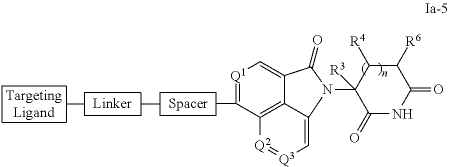

or a pharmaceutically acceptable salt thereof;
wherein Q¹, Q², and Q³ are independently selected from CH, CR¹, and N; and all other variables are as defined herein.
52. The compound of embodiment 1, wherein the compound is selected from:

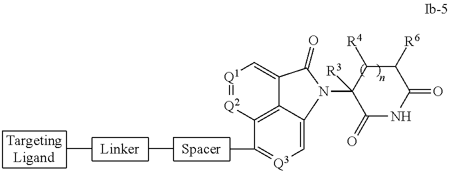

or a pharmaceutically acceptable salt thereof,
wherein Q¹, Q², and Q³ are independently selected from CH, CR¹, and N; and all other variables are as defined herein.
53. The compound of any one of embodiments 49-52, wherein Q¹ is CR¹.
54. The compound of any one of embodiments 49-52, wherein Q¹ is N.
55. The compound of any one of embodiments 49-54, wherein Q² is CR¹.
56. The compound of any one of embodiments 49-54, wherein Q² is N.
57. The compound of any one of embodiments 49-56, wherein Q³ is CR¹.
58. The compound of any one of embodiments 49-56, wherein Q³ is N.
59. The compound of any one of embodiments 1-52, wherein there is 3 R¹ substituent.
60. The compound of any one of embodiments 1-58, wherein there is 2 R¹ substituent.
61. The compound of any one of embodiments 1-58, wherein there is 1 R¹ substituent.
62. The compound of any one of embodiments 1-61, wherein R¹ is selected from alkyl, halogen, and haloalkyl.
63. The compound of any one of embodiments 1-61, wherein R¹ is selected from —OR¹⁰, —SR¹⁰, —S(O)R¹², —SO₂R¹², —NR¹⁰R¹¹.
64. The compound of any one of embodiments 1-61, wherein R¹ is selected from alkyl, halogen, and haloalkyl.
65. The compound of any one of embodiments 1-60, wherein two R¹ substituents are combined to form a fused phenyl ring.
66. The compound of any one of embodiments 1-61, wherein at least one R¹ is alkyl.
67. The compound of any one of embodiments 1-61, wherein at least one R¹ is halogen.
68. The compound of any one of embodiments 1-67, wherein R³ is hydrogen.
69. The compound of any one of embodiments 1-67, wherein R³ is alkyl.
70. The compound of any one of embodiments 1-67, wherein R³ is haloalkyl.
71. The compound of any one of embodiments 1-67, wherein R³ and R⁶ are combined to form a one carbon attachment.
72. The compound of any one of embodiments 1-67, wherein R³ and R⁶ are combined to form a two carbon attachment.
73. The compound of any one of embodiments 1-70, wherein R⁶ is hydrogen.
74. The compound of any one of embodiments 1-70, wherein R⁶ is alkyl.
75. The compound of any one of embodiments 1-70, wherein R⁶ is haloalkyl.
76. The compound of any one of embodiments 1-75, wherein at least one R⁴ is hydrogen.
77. The compound of any one of embodiments 1-75, wherein at least one R⁴ is alkyl.
78. The compound of any one of embodiments 1-75, wherein at least one R⁴ is haloalkyl.
79. The compound of any one of embodiments 1-75, wherein n is 0.
80. The compound of any one of embodiments 1-78, wherein n is 1.
81. The compound of any one of embodiments 1-78, wherein n is 2.
82. The compound of any one of embodiments 1-81, wherein Linker is of formula:

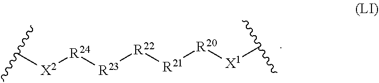

wherein,
X¹ and X² are independently at each occurrence selected from bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂— and —S—;
each of which heterocycle, aryl, heteroaryl, and bicycle is substituted with 1, 2, 3, or 4 substituents independently selected from R⁴⁰;
R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR⁷—, —NR²⁷C(O)—, —O—, —S—, —NR²⁷—, —C(R⁴⁰R⁴⁰)—, —P(O)(0R²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, and carbocycle; each of which is optionally substituted with 1, 2, 3, or 4 substituents independently selected from R⁴⁰;
R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, and cycloalkyl; and
R⁴¹ is aliphatic, aryl, heteroaryl, or hydrogen.
83. The compound of embodiment 82, wherein L is a linker of formula:

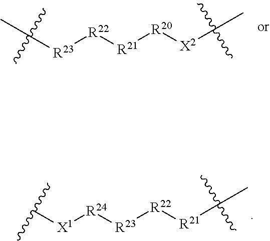

84. The compound of embodiment 82 or 83, wherein X¹ is bond.
85. The compound of embodiment 82 or 83, wherein X¹ is heterocycle.
86. The compound of embodiment 82 or 83, wherein X¹ is NR².
87. The compound of embodiment 82 or 83, wherein X¹ is C(O).
88. The compound of any one of embodiments 82-87, wherein X² is bond.
89. The compound of any one of embodiments 82-87, wherein X² is heterocycle.
90. The compound of any one of embodiments 82-87, wherein X² is NR².
91. The compound of any one of embodiments 82-87, wherein X² is C(O).
92. The compound of any one of embodiments 82-91, wherein R²⁰ is bond.
93. The compound of any one of embodiments 82-91, wherein R²⁰ is CH₂.
94. The compound of any one of embodiments 82-91, wherein R²⁰ is heterocycle.
95. The compound of any one of embodiments 82-91, wherein R²⁰ is aryl.
96. The compound of any one of embodiments 82-91, wherein R²⁰ is phenyl.
97. The compound of any one of embodiments 82-91, wherein R²⁰ is bicycle.
98. The compound of any one of embodiments 82-97, wherein R²¹ is bond.
99. The compound of any one of embodiments 82-97, wherein R²¹ is CH₂.
100. The compound of any one of embodiments 82-97, wherein R²¹ is heterocycle.
101. The compound of any one of embodiments 82-97, wherein R²¹ is aryl.
102. The compound of any one of embodiments 82-97, wherein R²¹ is phenyl.
103. The compound of any one of embodiments 82-97, wherein R²¹ is bicycle.
104. The compound of embodiment 83, wherein Linker is of formula:

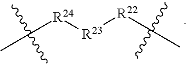

105. The compound of any one of embodiments 82-104, wherein R²² is bond.
106. The compound of any one of embodiments 82-104, wherein R²² is CH₂.
107. The compound of any one of embodiments 82-104, wherein R²² is heterocycle.
108. The compound of any one of embodiments 82-104, wherein R²² is aryl.
109. The compound of any one of embodiments 82-104, wherein R²² is phenyl.
110. The compound of any one of embodiments 82-104, wherein R²² is bicycle.
111. The compound of embodiment 82, wherein Linker is of formula:

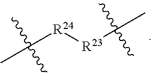

112. The compound of any one of embodiments 82-111, wherein R²³ is bond.
113. The compound of any one of embodiments 82-111, wherein R²¹ is CH₂.
114. The compound of any one of embodiments 82-111, wherein R²³ is heterocycle.
115. The compound of any one of embodiments 82-111, wherein R²³ is aryl.
116. The compound of any one of embodiments 82-111, wherein R²³ is phenyl.
117. The compound of any one of embodiments 82-111, wherein R²³ is bicycle.
118. The compound of embodiment 82, wherein Linker is of formula:

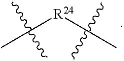

119. The compound of any one of embodiments 82-118, wherein R²⁴ is bond.
120. The compound of any one of embodiments 82-118, wherein R²⁴ is CH₂.
121. The compound of any one of embodiments 82-118, wherein R²⁴ is heterocycle.
122. The compound of any one of embodiments 82-118, wherein R²⁴ is aryl.
123. The compound of any one of embodiments 82-118, wherein R²⁴ is phenyl.
124. The compound of any one of embodiments 82-118, wherein R²⁴ is bicycle.
125. The compound of any one of embodiments 82-118, wherein R²⁴ is C(O).
126. The compound of any one of embodiments 1-125, wherein Linker is selected from:

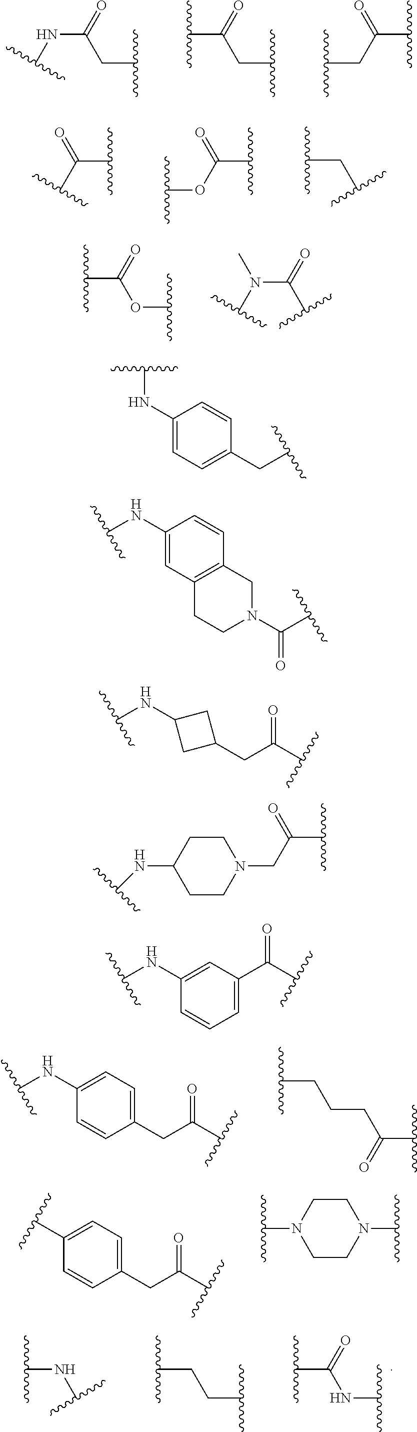

127. The compound of any one of embodiments 1-126, wherein Spacer is a bivalent connecting moiety of structure:

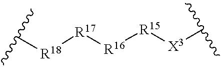

wherein:
X³ is a bivalent moiety selected from bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂— and —S—; or can be arylalkyl, heterocyclealkyl or heteroarylalkyl (in either direction), each of which heterocycle, aryl, heteroaryl, and bicycle may be substituted with 1, 2, 3, or 4 substituents independently selected from R⁴⁰;
R¹⁵, R¹⁶, R¹⁷, and R¹⁸ are independently at each occurrence selected from the group consisting of a bond, alkyl (which in certain embodiments is a carbocycle), —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, arylalkyl, heterocyclealkyl, and heteroarylalkyl; each of which is optionally substituted with 1, 2, 3, or 4 substituents independently selected from R⁴⁰.
128. The compound of embodiment 127, wherein L is a linker of formula:

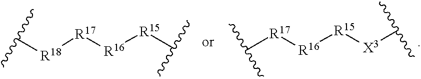

129. The compound of embodiment 128 or 129, wherein X³ is bond.
130. The compound of embodiment 128 or 129, wherein X³ is heterocycle.
131. The compound of embodiment 128 or 129, wherein X³ is NR².
132. The compound of embodiment 128 or 129, wherein X³ is C(O).
133. The compound of any one of embodiments 127-132, wherein R¹⁵ is bond.
134. The compound of any one of embodiments 127-132, wherein R¹⁵ is CH₂.
135. The compound of any one of embodiments 127-132, wherein R¹⁵ is heterocycle.
136. The compound of any one of embodiments 127-132, wherein R¹⁵ is aryl.
137. The compound of any one of embodiments 127-132, wherein R¹⁵ is phenyl.
138. The compound of any one of embodiments 127-132, wherein R¹⁵ is bicycle.
139. The compound of any one of embodiments 127-138, wherein R¹⁶ is bond.
140. The compound of any one of embodiments 127-138, wherein R¹⁶ is CH₂.
141. The compound of any one of embodiments 127-138, wherein R¹⁶ is heterocycle.
142. The compound of any one of embodiments 127-138, wherein R¹⁶ is aryl.
143. The compound of any one of embodiments 127-138, wherein R¹⁶ is phenyl.
144. The compound of any one of embodiments 127-138, wherein R¹⁶ is bicycle.
145. The compound of any one of embodiments 127-144, wherein R¹⁷ is bond.
146. The compound of any one of embodiments 127-144, wherein R¹⁷ is CH₂.
147. The compound of any one of embodiments 127-144, wherein R¹⁷ is heterocycle.
148. The compound of any one of embodiments 127-144, wherein R¹⁷ is aryl.
149. The compound of any one of embodiments 127-144, wherein R¹⁷ is phenyl.
150. The compound of any one of embodiments 127-144, wherein R¹⁷ is bicycle.
151. The compound of any one of embodiments 127-150, wherein R¹⁸ is bond.
152. The compound of any one of embodiments 127-150, wherein R¹⁸ is CH₂.
153. The compound of any one of embodiments 127-150, wherein R¹⁸ is heterocycle.
154. The compound of any one of embodiments 127-150, wherein R¹⁸ is aryl.
155. The compound of any one of embodiments 127-150, wherein R¹⁸ is phenyl.
156. The compound of any one of embodiments 127-150, wherein R¹⁸ is bicycle.
157. The compound of any one of embodiments 1-156, wherein Targeting Ligand is selected from a structure in the figures, wherein the Targeting Ligand is optionally substituted with 1, 2, 3, or 4 R⁴⁰ substituents.
158. A pharmaceutical composition comprising a compound of any one of embodiments 1-157 or a pharmaceutically acceptable salt thereof and a pharmaceutically acceptable carrier.
159. A method of treating a disorder that is not mediated by the Target Protein in a patient in need thereof comprising administering an effective amount of a compound of any one of embodiments 1-157 or a pharmaceutical composition of embodiment 158.
160. A method of treating a disorder that is mediated by the Target Protein in a patient in need thereof comprising administering an effective amount of a compound of any one of embodiments 1-157 or a pharmaceutical composition of embodiment 158.
161. A method of treating a disorder mediated by a Target Protein in a patient in need thereof comprising administering an effective amount of a compound of any one of embodiments 1-157 or a pharmaceutical composition of embodiment 158.
162. The method of embodiment 159, 160, or 161, wherein the disorder is abnormal cellular proliferation.
163. The method of embodiment 159, 160, or 161, wherein the disorder is a neurodegenerative disorder 164. The method of embodiment 159, 160, or 161, wherein the disorder is an immune system disorder.
165. The method of any one of embodiments 159-164, wherein the patient is a human.

### III. Spacers

In certain embodiments, Spacer is selected from:

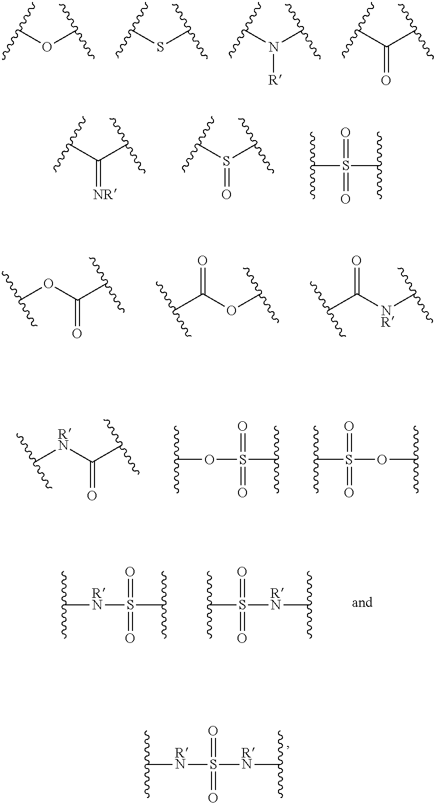

wherein each R′ is independently selected from hydrogen, alkyl, haloalkyl, aryl, heterocycle, and heteroaryl.

In certain embodiments, Spacer is selected from:

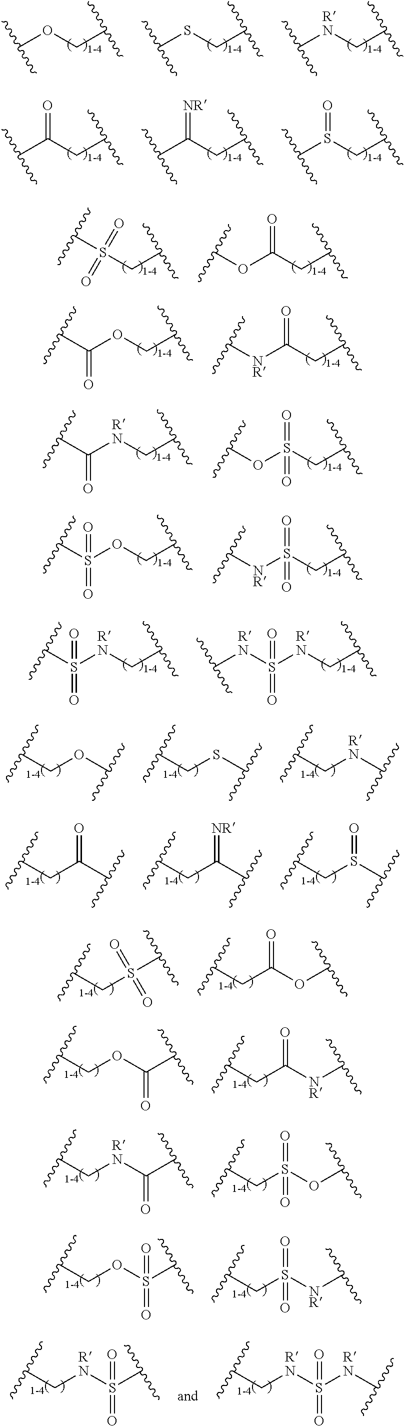

In certain embodiments, Spacer is selected from linear alkyl chains of 1-5 carbons in length, optionally bearing one or more degrees of unsaturation where valence allows.

In certain embodiments, Spacer is selected from:

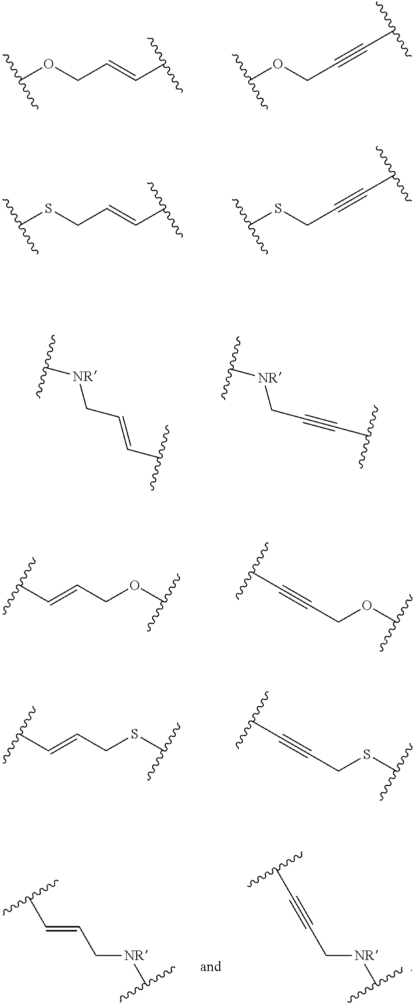

In certain embodiments, Spacer is a heterocycle group optionally substituted with 1 or 2 substituents selected from R′.

In certain embodiments, Spacer is a 6-membered heterocycle group with one or two nitrogen atoms.

In certain embodiments, Spacer is a 6-membered heterocycle group with one or two oxygen atoms

In certain embodiments, Spacer is selected from:

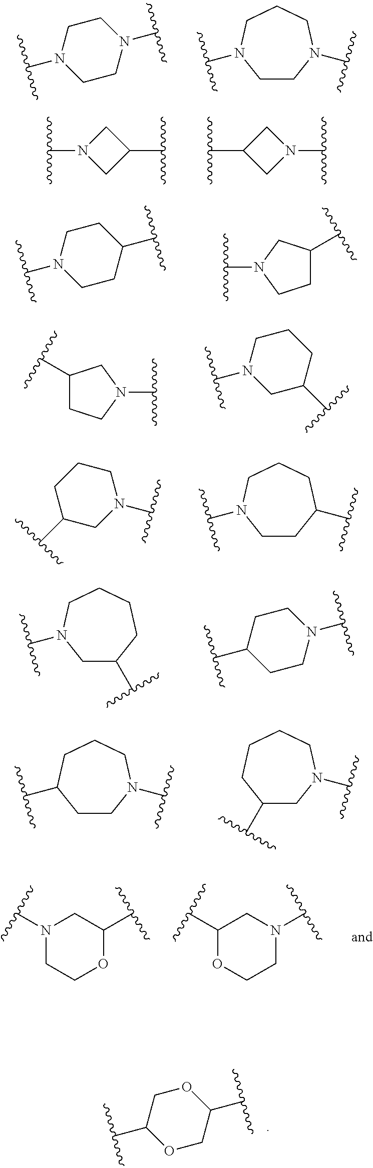

In certain embodiments, Spacer is selected from:

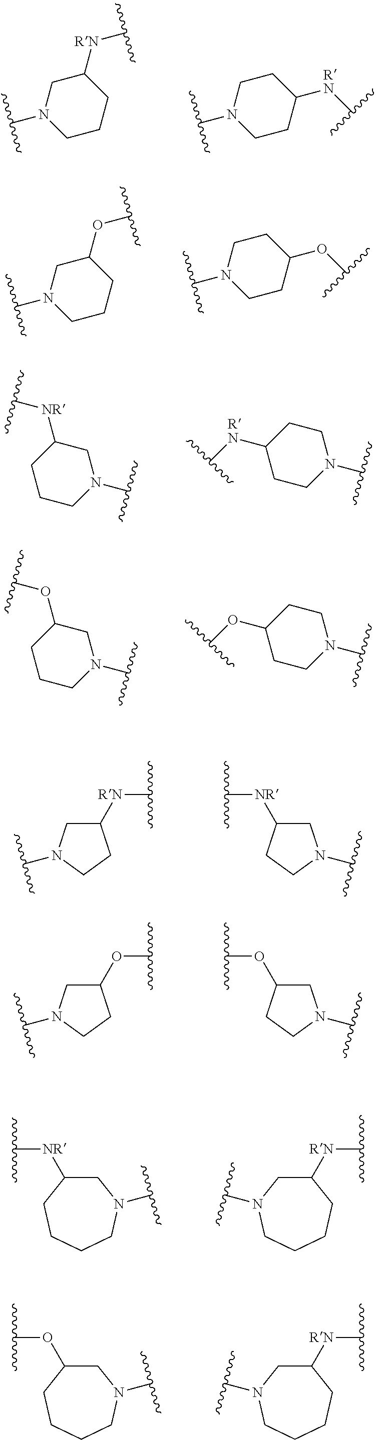

In certain embodiments, Spacer is selected from:

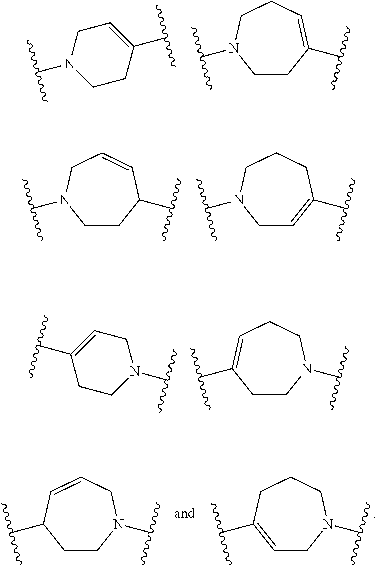

In certain embodiments, Spacer is selected from:

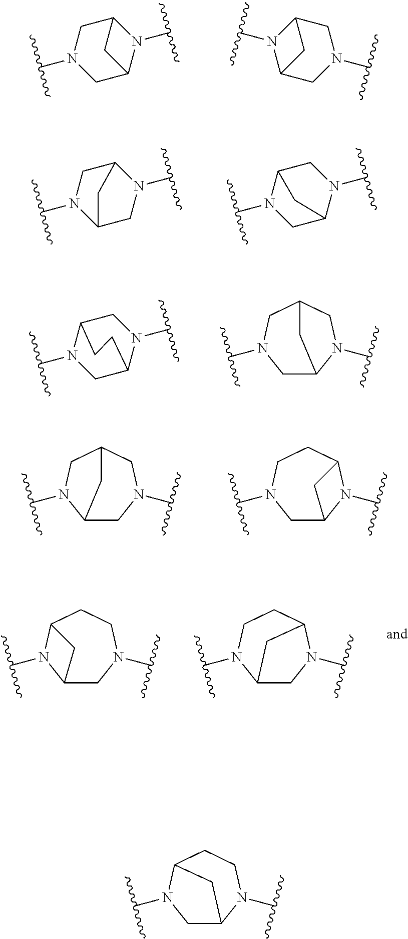

In certain embodiments, Spacer is selected from:

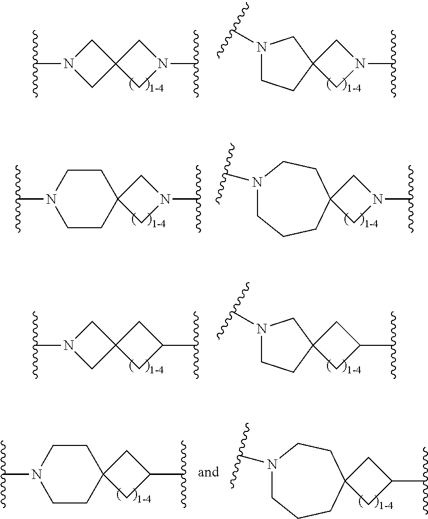

In certain embodiments, Spacer is selected from:

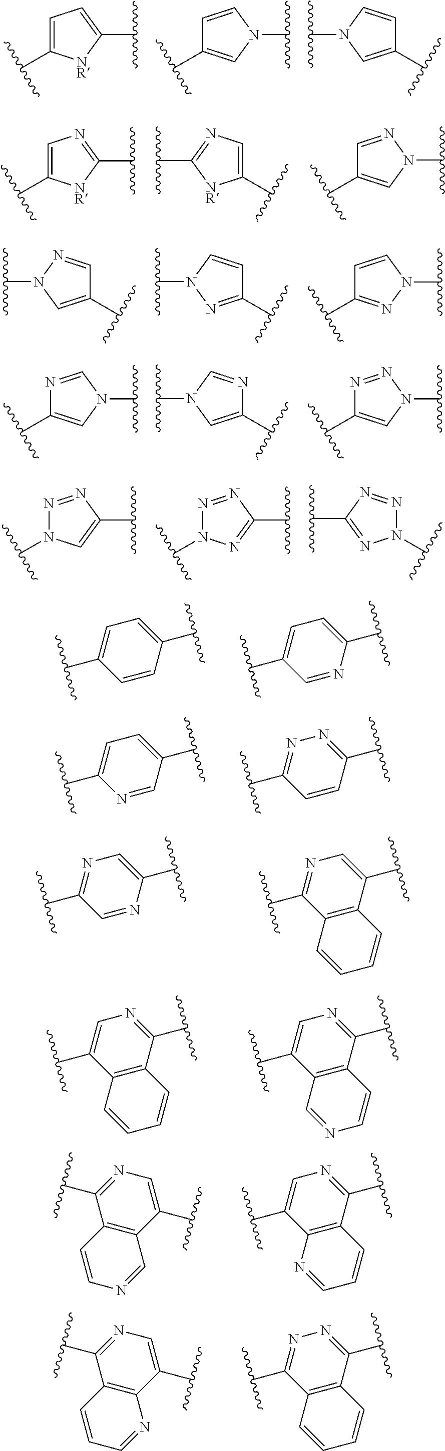

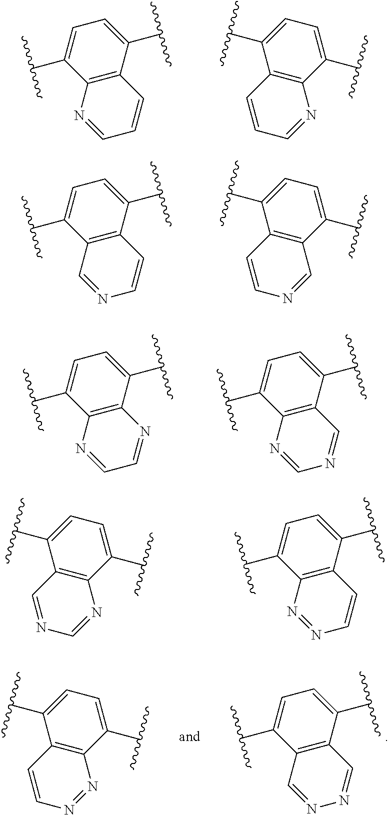

In certain embodiments, Spacer is selected from:

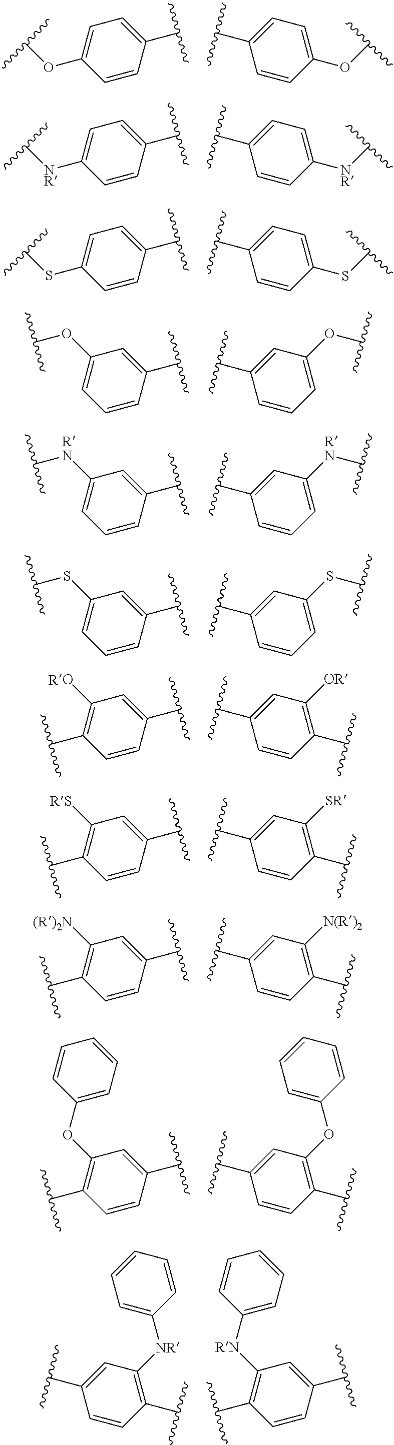

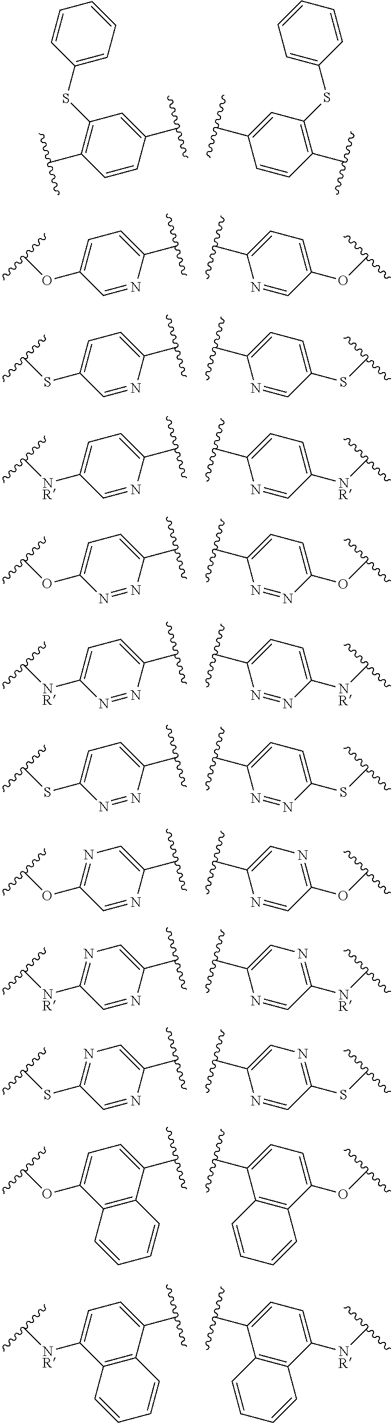

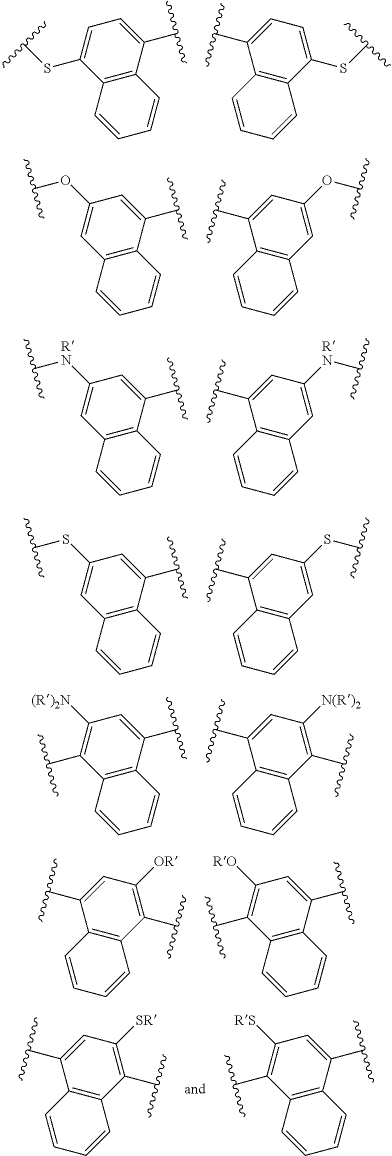

In certain embodiments, Spacer is selected from:

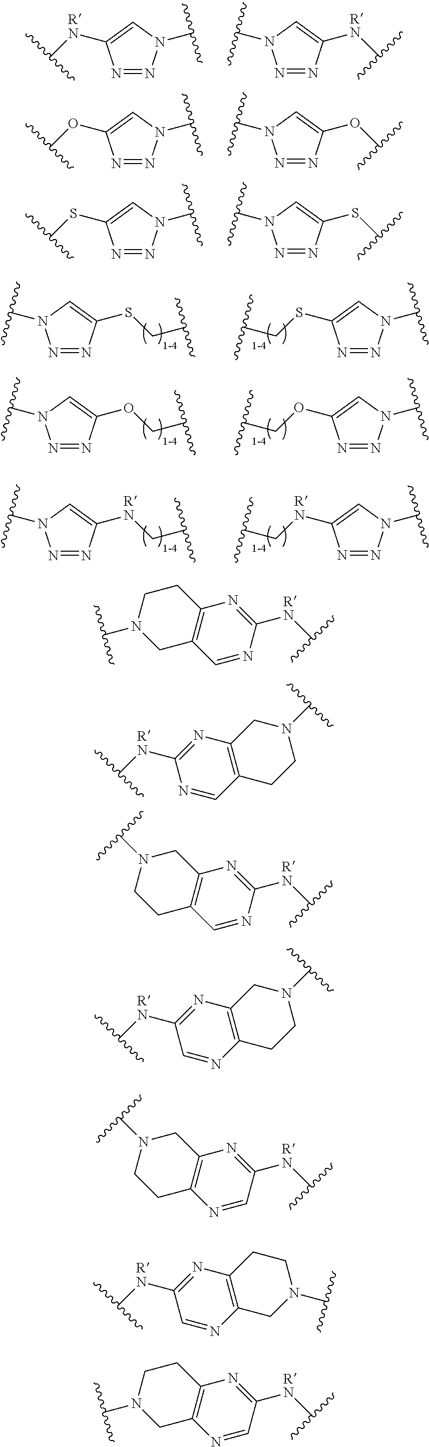

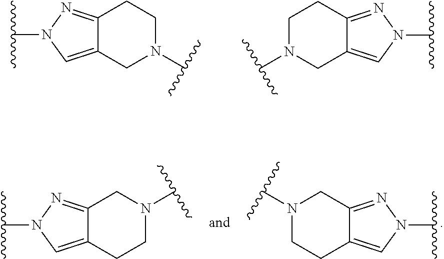 [SMILES: *N1CCc2nn(*)cc2C1 ; *N1CCc2nn(*)cc2C1 ; *N1CCc2cn(*)nc2C1 ; *N1CCc2cn(*)nc2C1]

In certain embodiments, Spacer is selected from:

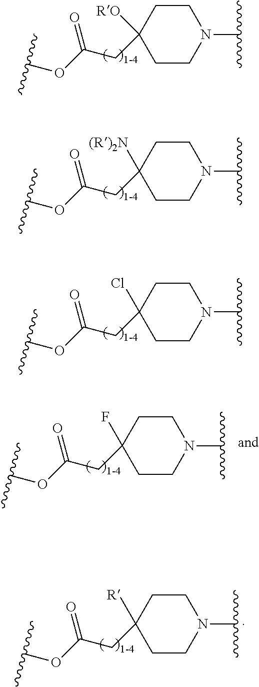

In certain embodiments, Spacer is selected from:

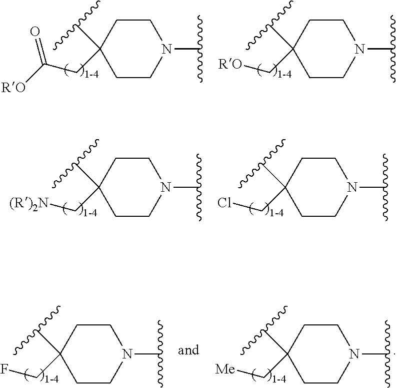

In certain embodiments of Spacer, the bond to the left in the drawings above is connected to the linker.

In other embodiments of Spacer, the bond to the right in the drawings above is connected to the linker.

In certain embodiments, the compound of the present invention is selected from:

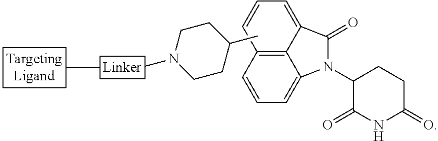

In the structures herein, when a bond is floating on one of two fused cycles the bond can be attached at any appropriate position on either Cycle-A s allowed by valence. For example, non-limiting examples of

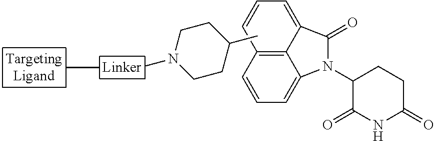

include both

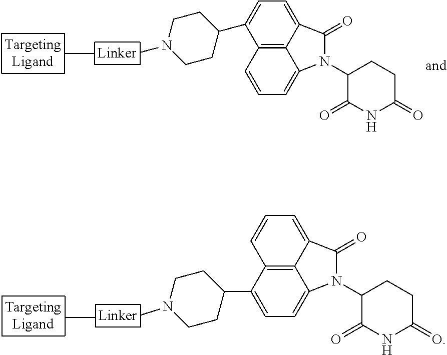

### IV. Linkers

A Linker is included in the compounds of Formula I, Formula II, or Formula III. Linker is a chemically stable bivalent group that attaches an E3 Ligase binding portion to a Targeting Ligand. According to the invention, any desired linker, as described herein, can be used, as long as the resulting compound has a stable shelf life for at least 2 months, 3 months, 6 months or 1 year as part of a pharmaceutically acceptable dosage form, and itself is pharmaceutically acceptable.

Linker as described herein can be used in either direction, i.e., either the left end is linked to the Spacer portion and the right end to the Targeting Ligand, or the left end is linked to the Targeting Ligand and the right end is linked to the Spacer portion.

In certain embodiments, the Linker has a chain of 2, 3, 4, 5, 6, 7, 8, 9, 10, 11, 12, 13, 14, 15, 16, 17, 18 or 20 or more carbon atoms of which one or more carbons can be replaced by a heteroatom such as O, N, S, or P.

In certain embodiments the chain has 2, 3, 4, 5, 6, 7, 8, 9, 10, 11, 12, 13, 14, 15, 16, 17, 18, 19 or 20 contiguous atoms in the chain. For example, the chain may include 1 or more ethylene glycol units that can be contiguous, partially contiguous or non-contiguous (for example, 2, 3, 4, 5, 6, 7, 8, 9, 10, 11 or 12 ethylene glycol units).

In certain embodiments the chain has at least 1, 2, 3, 4, 5, 6, 7, or 8 contiguous chains which can have branches which can be independently alkyl, aryl, heteroaryl, alkenyl, or alkynyl, aliphatic, heteroaliphatic, cycloalkyl or heterocycle substituents.

In other embodiments, the linker can include or be comprised of one or more of ethylene glycol, propylene glycol, lactic acid and/or glycolic acid. In general, propylene glycol adds hydrophobicity, while propylene glycol adds hydrophilicity. Lactic acid segments tend to have a longer half-life than glycolic acid segments. Block and random lactic acid-co-glycolic acid moieties, as well as ethylene glycol and propylene glycol, are known in the art to be pharmaceutically acceptable and can be modified or arranged to obtain the desired half-life and hydrophilicity. In certain aspects, these units can be flanked or interspersed with other moieties, such as aliphatic, including alkyl, heteroaliphatic, aryl, heteroaryl, heterocycle, cycloalkyl, etc., as desired to achieve the appropriate drug properties.

In certain embodiments, Linker is selected from:

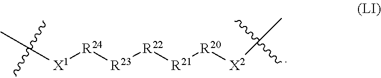

In one aspect, Linker is selected from the group consisting of a moiety of Formula LI, Formula LII, Formula LIII, Formula LIV, Formula LV, Formula LVI, Formula LVII Formula LVIII, Formula IX and Formula LX:

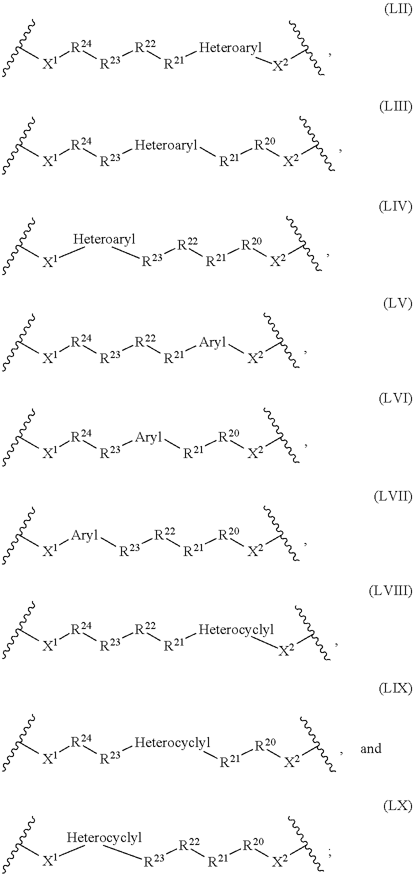

In certain embodiments, Linker selected from:

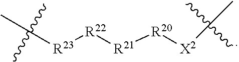

In one aspect, Linker is selected from the group consisting of a moiety of Formula LDI, Formula LDII, Formula LDIII, Formula LDIV, Formula LDV, Formula LDVI, and Formula LDVII:

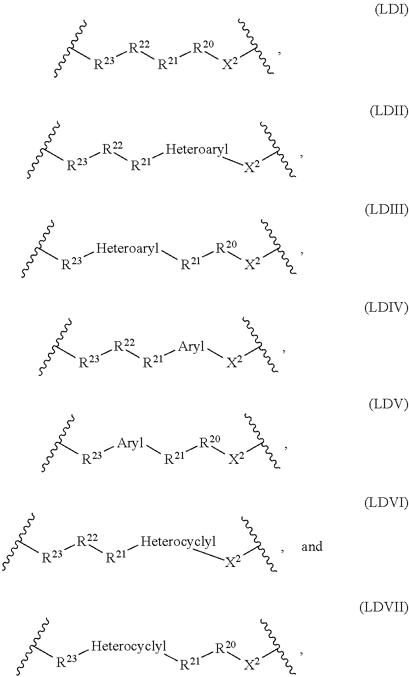

wherein all variables are described herein.

The following are non-limiting examples of Linkers that can be used in this invention.

Based on this elaboration, those of skill in the art will understand how to use the full breadth of Linkers that will accomplish the goal of the invention.

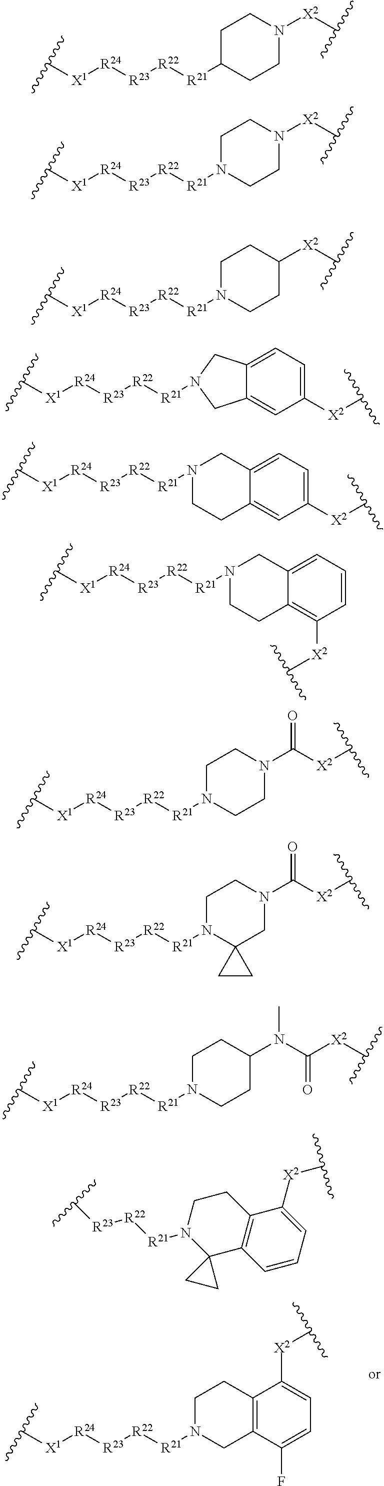

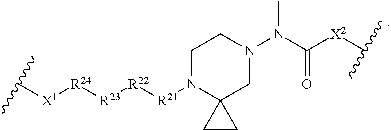

Non-limiting examples of Linker include:

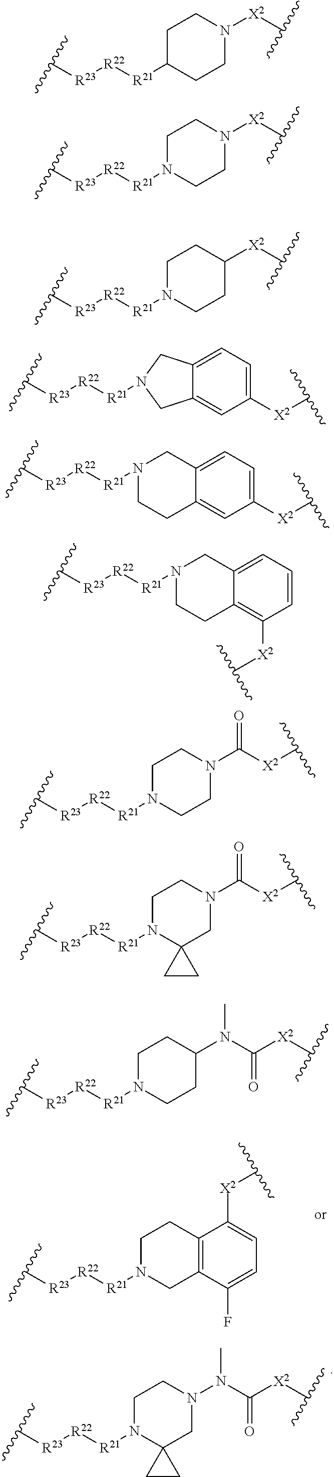

In certain embodiments X² is attached to the Targeting Ligand. In another embodiment X is attached to the Targeting Ligand.

Non-limiting examples of moieties of R²⁰, R²¹, R²², R²³, and R²⁴ include:

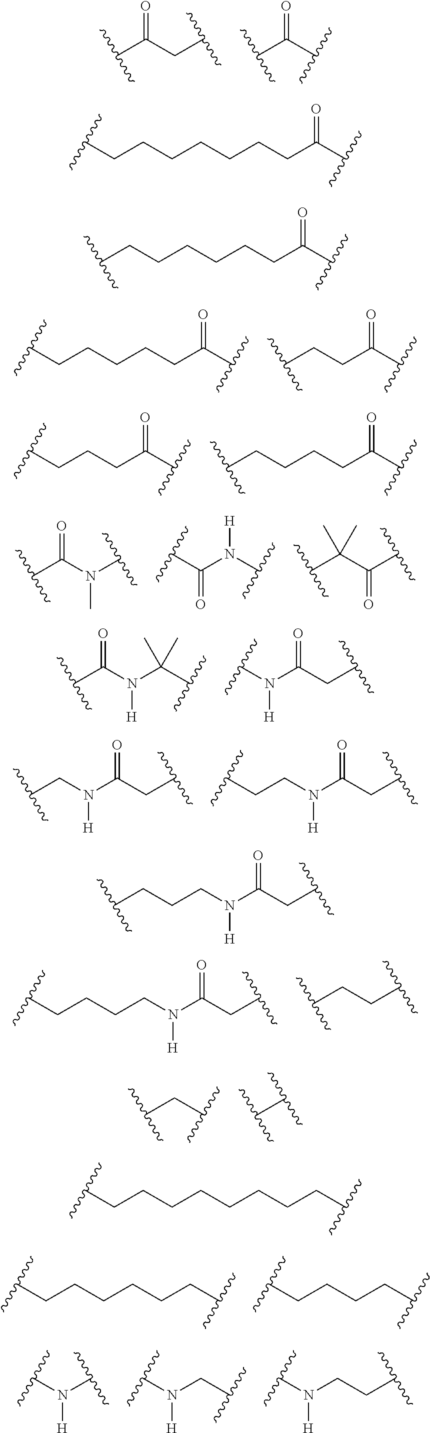

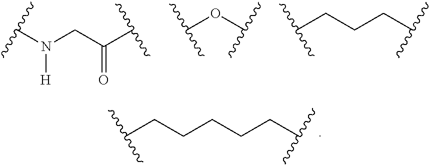

Additional non-limiting examples of moieties of R²⁰, R²¹, R²², R²³ and R²⁴ include:

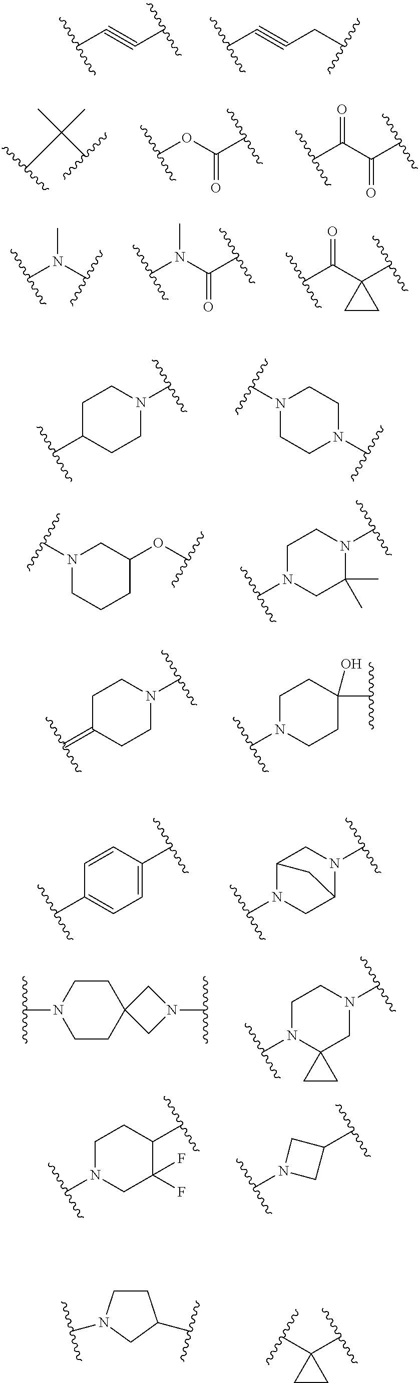

Additional non-limiting examples of moieties of R²⁰, R²¹, R²², R²³ and R²⁴ include:

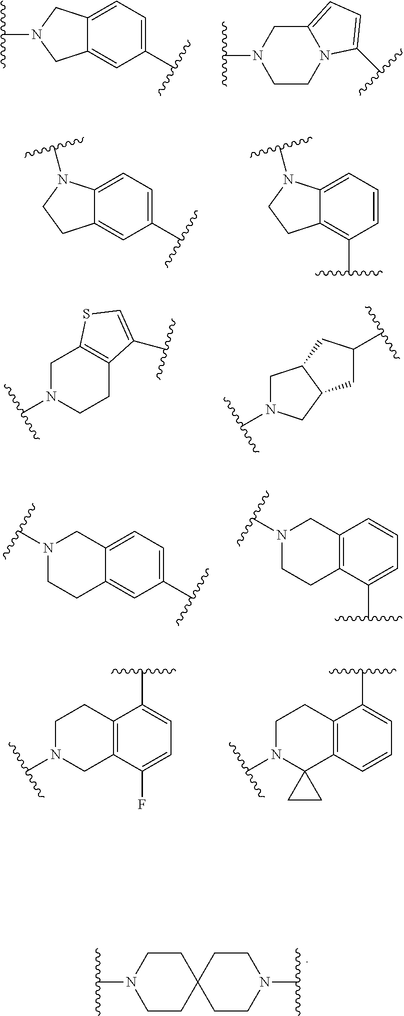

In additional embodiments, the Linker moiety is an optionally substituted (poly)ethylene glycol having at least 1, at least 2, at least 3, at least 4, at least 5, at least 6, at least 7, at least 8, at least 9, at least 10, ethylene glycol units, or optionally substituted alkyl groups interspersed with optionally substituted, O, N, S, P or Si atoms.

In certain embodiments, the Linker is flanked, substituted, or interspersed with an aryl, phenyl, benzyl, alkyl, alkylene, or heterocycle group.

In certain embodiments, the Linker may be asymmetric or symmetrical.

In certain embodiments, Linker can be a nonlinear chain, and can be, or include, aliphatic or aromatic or heteroaromatic cyclic moieties.

In any of the embodiments of the compounds described herein, the Linker group may be any suitable moiety as described herein.

In certain embodiments, Linker is selected from the group consisting of:

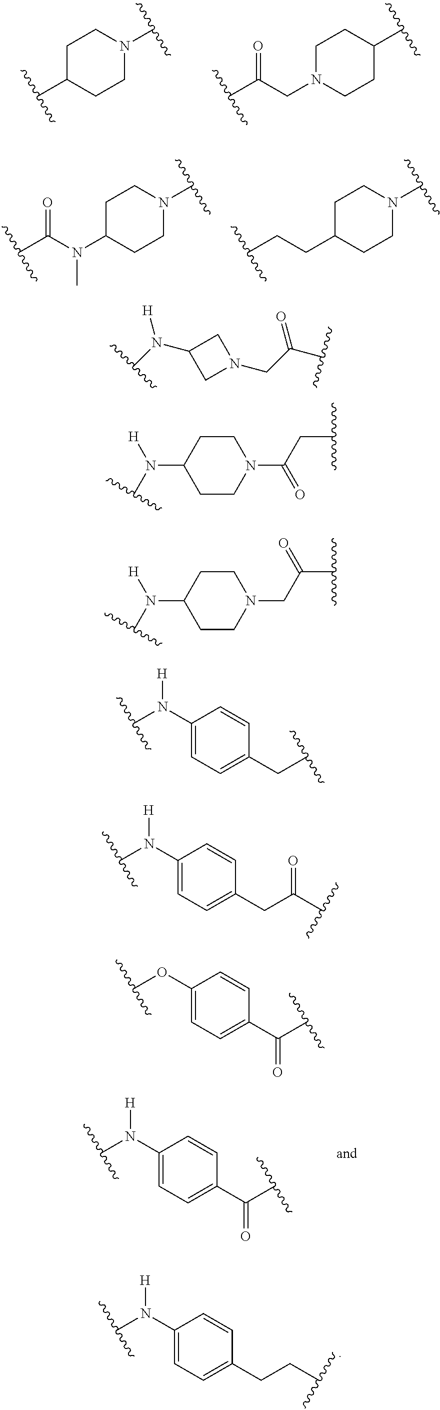

In certain embodiments, Linker is selected from the group consisting of:

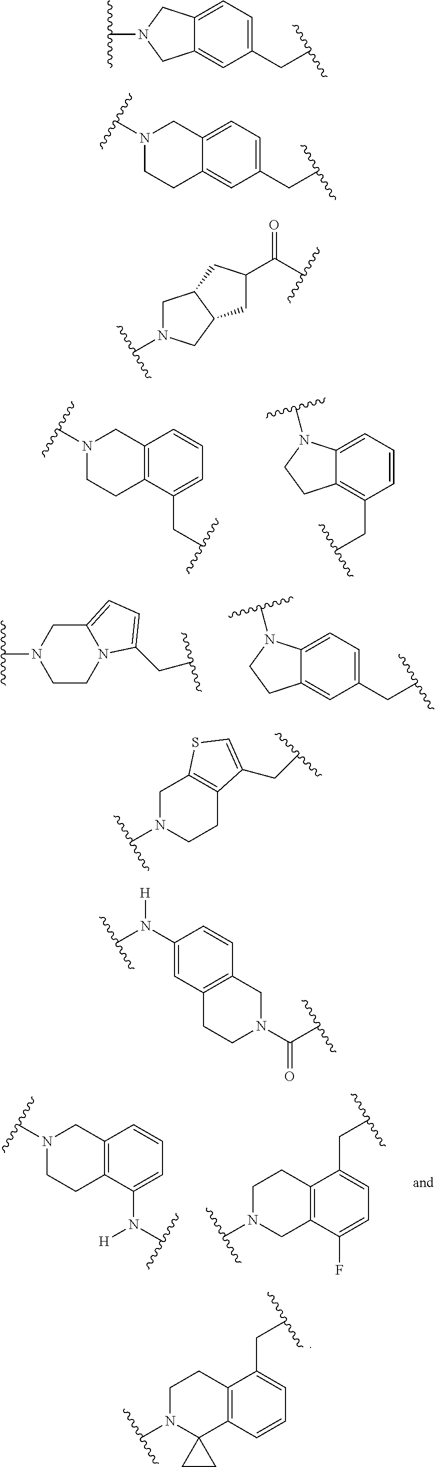

In certain embodiments, Linker is selected from the group consisting of:

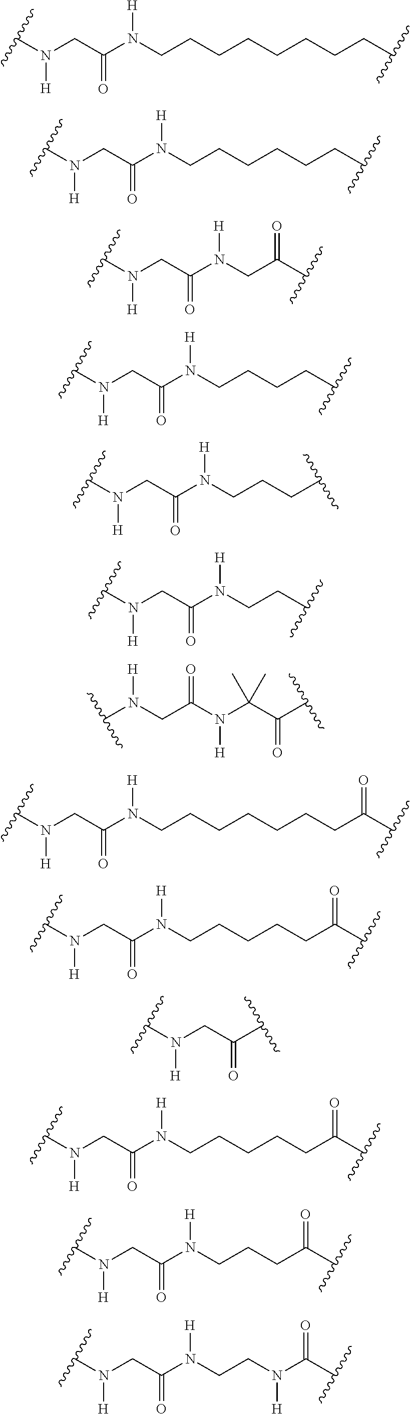

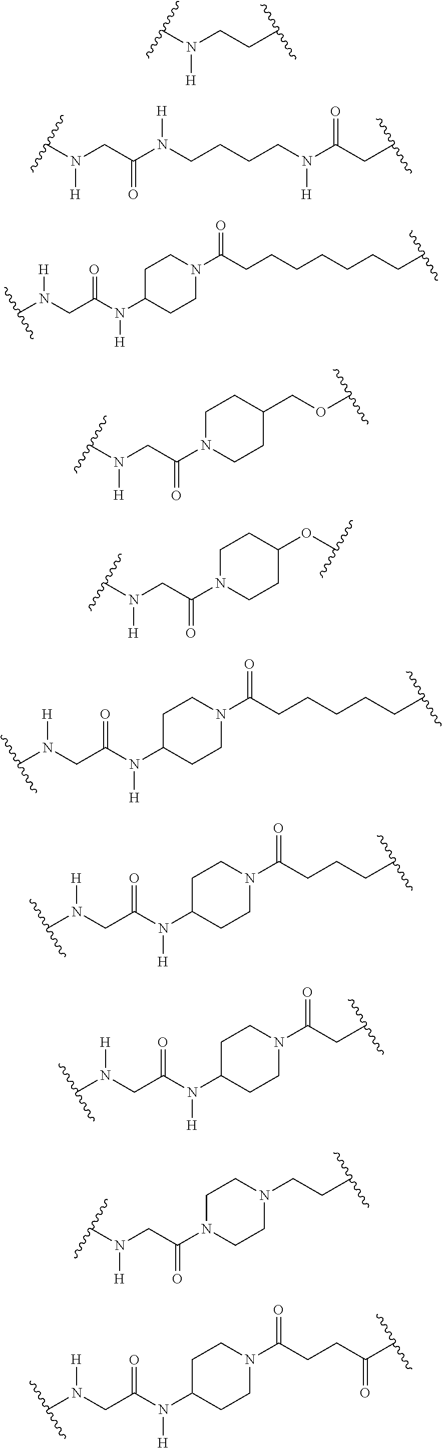

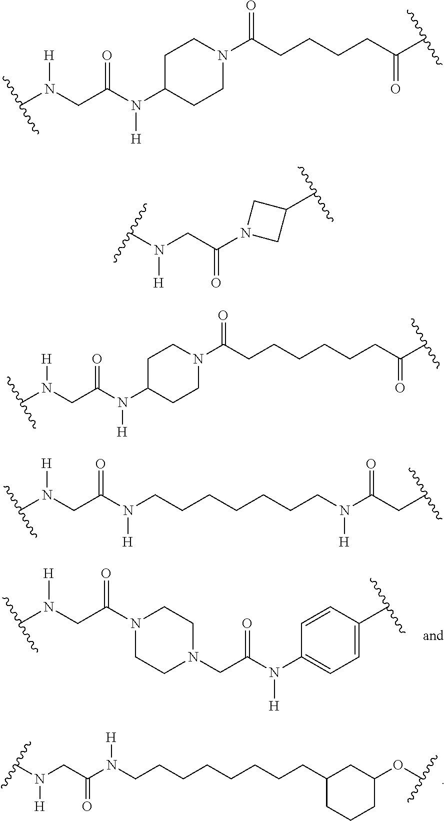

In certain embodiments, Linker is selected from the group consisting of

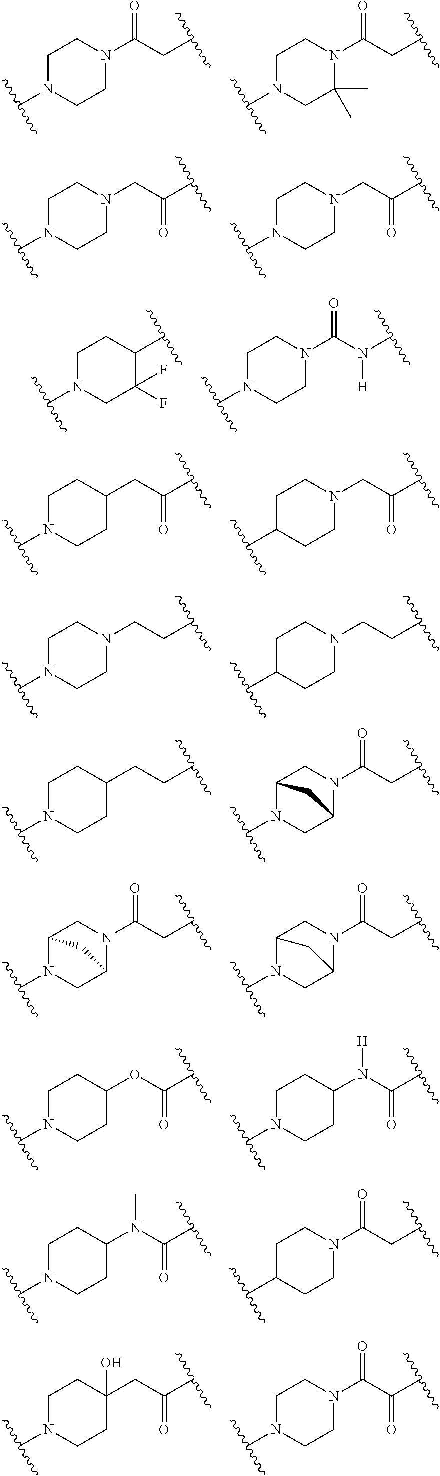

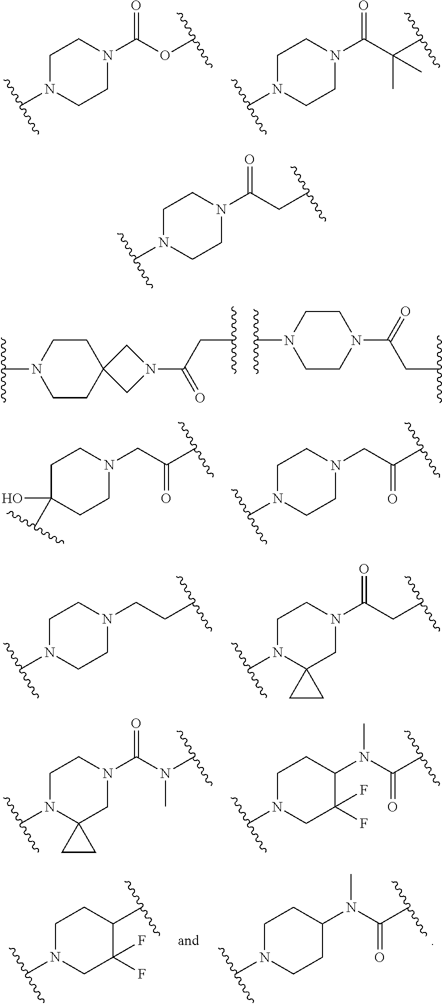

In certain embodiments, Linker is selected from the group consisting of:

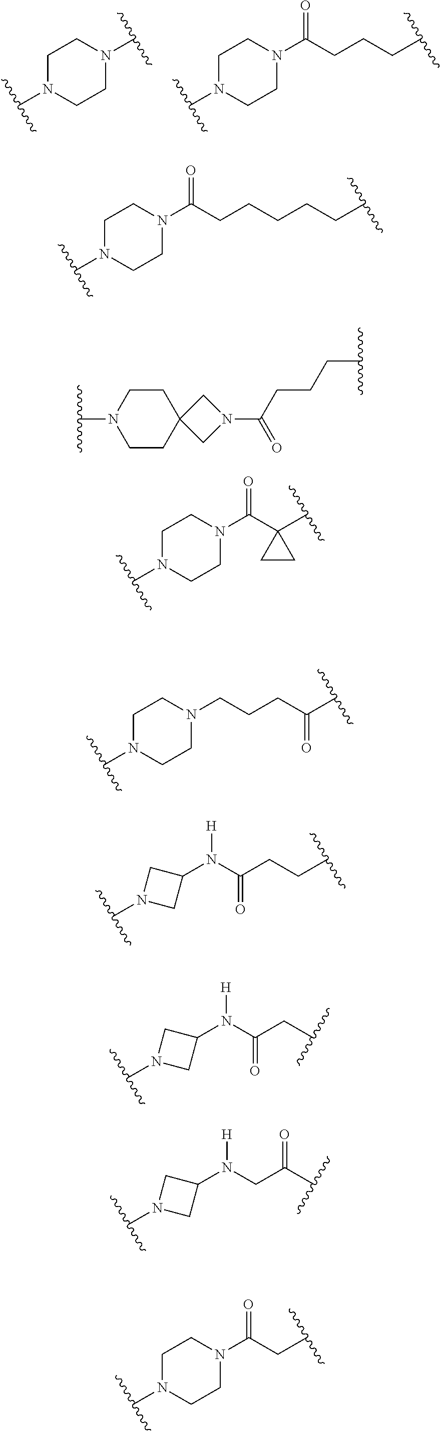

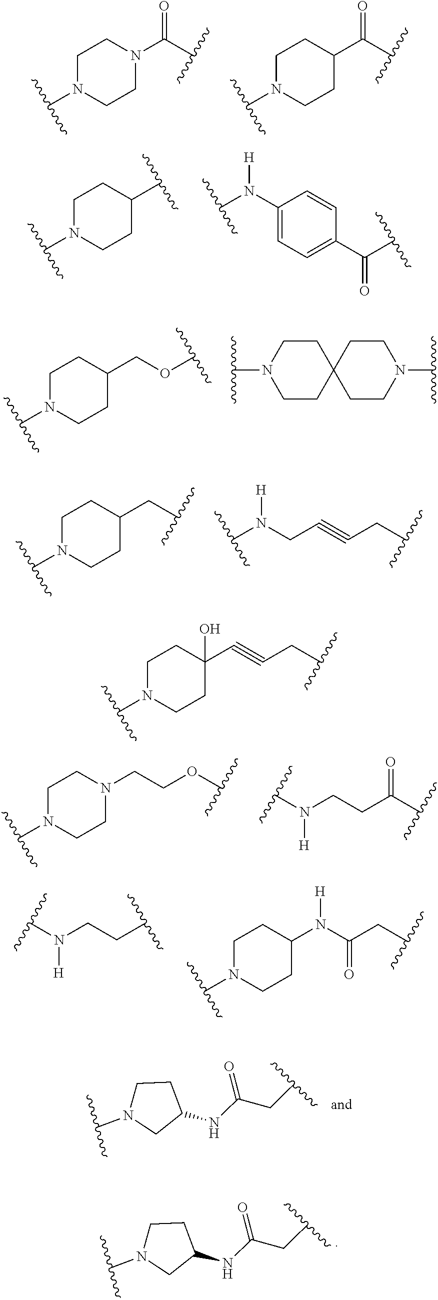

In certain embodiments Linker is selected from:

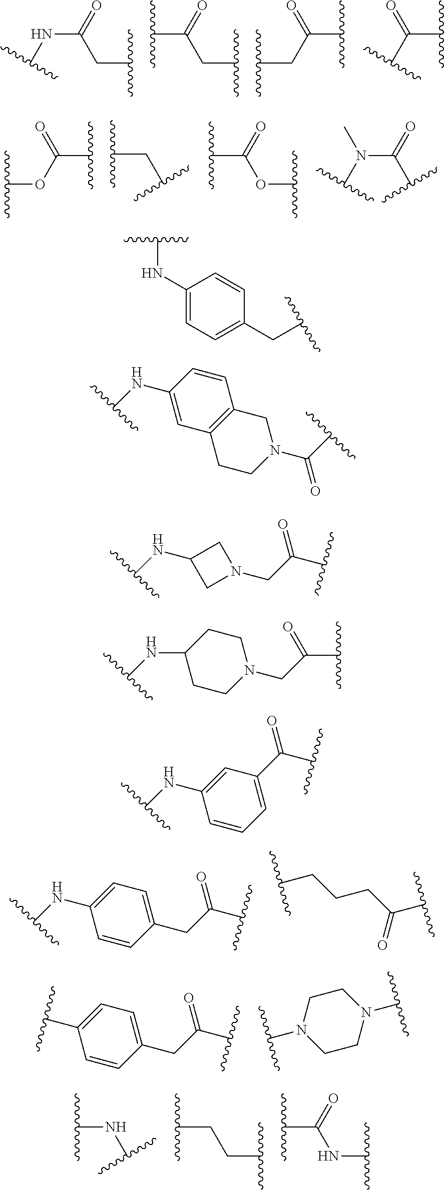

In certain embodiments, the Linker is selected from

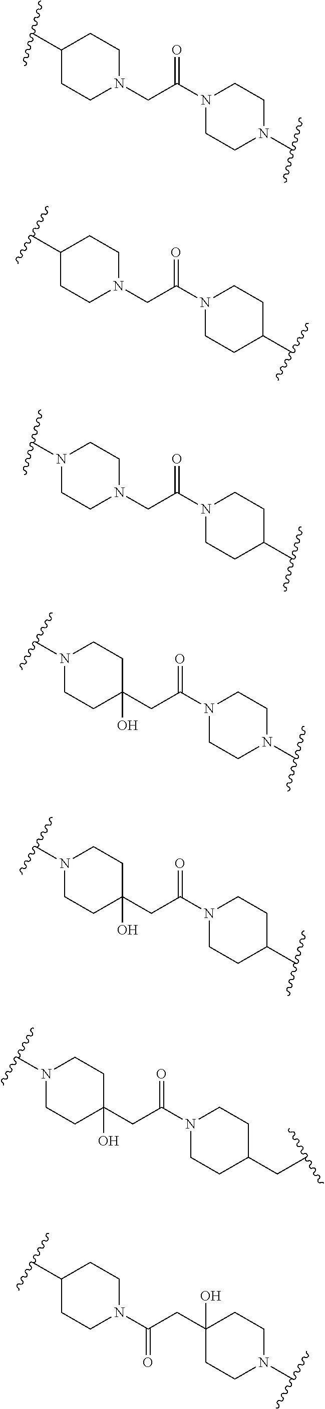

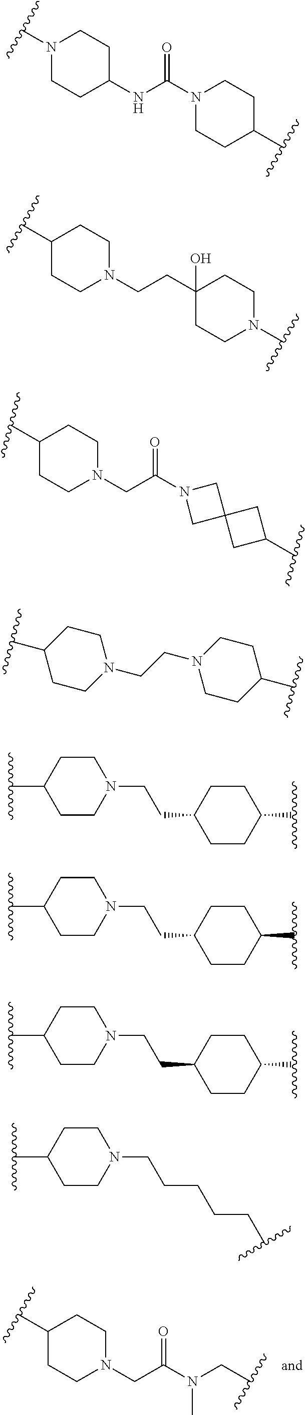

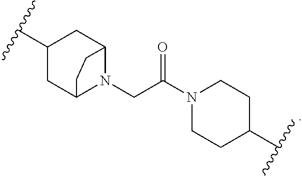 [SMILES: *C1CCN(C(=O)CN2C3CCC2CC(*)C3)CC1]

In certain embodiments the right bond of the Linker drawn above is attached to the Targeting Ligand. In certain embodiments the left bond of the Linker drawn above is attached to the Targeting Ligand.

### V. Target Proteins

Degradation of cellular proteins is required for cell homeostasis and normal cell function, such as proliferation, differentiation and cell death. When this system becomes dysfunctional or does not identify and abate abnormal protein behavior in vivo, a disease state can arise in a host, such as a human. A large range of proteins can become dysfunctional and cause, modulate or amplify diseases in vivo, as well known to those skilled in the art, published in literature and patent filings as well as presented in scientific presentations.

Therefore, in certain embodiments, a selected tricyclic cereblon binding heterobifunctional degrader compound of the present invention can be administered in vivo in an effective amount to a host in need thereof to catalytically degrade a selected protein that mediates a disorder to be treated. The selected protein target may modulate a disorder in a human via a mechanism of action such as modification of a biological pathway, pathogenic signaling or modulation of a signal cascade or cellular entry. It may be mutated, altered or overexpressed. In certain embodiments, the Target Protein is a protein that is not druggable in the classic sense in that it does not have a binding pocket or an active site that can be inhibited or otherwise bound, and cannot be easily allosterically controlled. In another embodiment, the Target Protein is a protein that is druggable in the classic sense, yet for therapeutic purposes, degradation of the protein is preferred to inhibition.

Specific examples of Target Proteins that can be targeted for degradation by the tricyclic compounds of the present invention with rationales for degradation include, but are not limited to the following:
CK1α is a casein kinase that is utilizes acidic proteins such as caseins as phosphorylation substrates. CK1A participates in Wnt signaling and its overexpression is correlated with poor survival in cancer.
GSPT1 (G1 to S phase transition 1) is a translation termination factor. GSPT1 interacts with BIRC2 and is proteolytically processed into an IAP-binding protein. GSPT1 is expressed in cancer tissues including in gastric cancers.
STAT proteins are cytoplasmic transcription factors that can be activated by various extracellular signaling proteins. Stat proteins have been shown to upregulate various genes involved in uncontrolled cellular proliferation, anti-apoptotic responses and/or angiogenesis.
SALL4 (Spalt Like Transcription Factor 4) is a developmental transcription factor which is associated with developmental syndromes and abnormalities such as Duane-Radial Ray Syndrome and Ivic Syndrome.
PLZF (promyelocytic leukemia zinc finger), also known as ZBTB16 (Zinc Finger and BTB Domain Containing 16) is a transcription factor that regulates cellular proliferation, differentiation, organ development, cell maintenance, and immune cell development.

PLZF acts as a tumor suppressor in some cancers, however, PLZF is actually an oncoprotein in certain cancers, such as renal cell carcinoma, glioblastoma, and testicular cancer.
p63 (tumor protein p63) is a pleiotropic protein involved in cellular proliferation, apoptosis, differentiation, and even aging. There are several isoforms of p63. Some forms of p63 will suppressor tumors while other forms and mutants will promote cancer metastasis.
NRAS is a small GTP binding protein in which oncogenic activating mutations drive tumorigenesis. NRAS mutations are found for example in melanoma and thyroid cancer, and sometimes occur at Q61K and Q61R
BRD9 (bromodomain-containing protein 9) is a constituent part of the SWI/SNF (BAF) chromatin remodeling complexes. Mutations in BRD9 have been linked to several cancers and even native BRD9 when overexpressed can be oncogenic. Cancers related to BRD9 include cervical cancer, non-small cell lung cancer, and liver cancer.
P13KCA is a kinase that is one of the most commonly mutated oncogenes across a variety of human cancers including but not limited to breast, endometrial, squamous head and neck and squamous lung. Mutations for example are H1047R, E545K, E542K and sometimes lead to aberrant activation of PI3K-AKT-mTOR pathway.
RET (RET proto-oncogene) is a protein that spans the cellular membrane and interacts with the cell's environment. RET binds growth factors and triggers complex cascades of chemical reactions within the cell. Non-limiting examples of RET mediated disorders include nonsyndromic paraganglioma, Hirschsprung disease, multiple endocrine neoplasia, lung cancer, and other cancers.
RIT1 is a small GTP binding protein, which is an activating mutation in cancers for example Noonan syndrome (a RAS-opathy), lung cancer and heme malignancies.
MCL1 is a member of the BCL2 family and regulator of apoptosis. Diseases that are associated with MCL1 include myeloid leukemia and chlamydia.
ARID1B is an AT-rich interactive domain-containing protein1. It is a component of SW1/SNF complex and binds DNA non-specifically. It is a top dependency specific to ARID1A mutated cancer cell lines. ARID1A-deficient cancers comprise a high percentage of certain tumors including but not limited to ovarian clear cell carcinoma.
P300 (histone acetyltransferase p300 or p300 HAT) is an enzyme that regulates transcription of genes via chromatin remodeling by mediating the wrapping of histone to DNA. As a result, P300 plays a vital role in cell growth and division. Mutations of P300 can cause various types of cancers including colon, stomach, breast and pancreatic cancer.
ARID2 is a component of the polybromo-associated BAF (PBAF) chromatin remodeling complex
FAM38 (also known as PIEZO1) is a pore-forming subunit of a non-specific cation channel. As a cation channel subunit FAM38 is involved in the recruitment of R-Ras to the endoplasmic reticulum. Loss of FAM38 has been shown to cause metathesis in small cell lung cancer lines.
NSD2, which belongs to a histone methyltransferase (“HMT”) gene class, is overexpressed by oncogenic fusion transcripts of for example, multiple myeloma, ALL, CLL and MCL.
CSK is a non-receptor tyrosine-protein kinase involved in the regulation of cell growth, differentiation, migration, and immune response.
CBLB is a E3 ubiquitin-protein ligase that accepts ubiquitin from E2 ubiquitin-conjugating enzymes and then transfers it to substrates for degradation.
EGFR (Epidermal Growth Factor Receptor) is a tyrosine kinase receptor. EGFR is associated with the progression of several epithelial malignancies including colorectal cancer, adenocarcinomas (including that of the lung), glioblastoma, and epithelial tumors of the head and neck. Additionally, EGFR can be used as a receptor for entry of a microbial infection or virus such as HCV.
WRN is a RecQ DNA helicase. WRN loss leads to DNA damage in MSI (microsatellite instability) cells, but not MSS (microsatellite stable) cells. This can lead to DSB responses to promote cell death and cell cycle arrest preferentially in MSI cells.
NTRK and its gene fusions (including NTRK1, NTRK2, and NTRK3 gene fusions) are oncogenes for several adult and pediatric cancers. NTRK fusions are a major source of rare cancers such as secretory breast carcinoma, mammary analogue secretory carcinoma, and infantile fibrosarcoma. NTRK fusions can also cause more common cancers as well.
ADAR is RNA specific adenosine deaminase. IFN-stimulated (ISG) signature-positive cancer cells are sensitive to the loss of ADAR, a dsRNA-editing enzyme that is also an ISG. Tumor-derived IFN resulting in chronic signaling creates a cellular state primed to respond to dsRNA accumulation, rendering ISG-positive tumors susceptible to ADAR loss. Loss of ADAR1 overcomes resistance to PD-1 checkpoint blockade caused by inactivation of antigen presentation.
SOS1 promotes generation of active form of KRAS so blocking can counteract upstream or mutant activation of KRAS.
KRAS is a gene that encodes K-Ras which is a protein in the RAS/MAPK pathway that relays extracellular signals to the cell's nucleus. These signals either result in proliferation or differentiation. K-Ras sends signals when it is bound to GTP which acts like a molecular on off switch. KRAS mutations are frequently observed in cecal cancers. K-Ras is implicated in several cancers including colorectal cancer and lung cancer.
WDR5 is a member of the WD repeat protein family. WD repeats are minimally conserved regions of 40 amino acids bracketed by gly-his and trp-asp. WDR5 interacts with host cell factor C1, MLL, and is a key determinant for MYC recruitment. WD5 is implicated in mixed lineage leukemias.
ALK, including ALK-fusions such as EML-ALK and ALK fusion proteins in which the kinase domain of ALK has been fused to the amino-terminal portion of various proteins have been described in numerous cancers including but not limited to ALCL, IMT, DLBCL, NSCLC, RMC, RCC, breast cancer, colon carcinoma, serous ovarian carcinoma (SOC) and esophageal squamous cell carcinoma (ESCC).
PTPN2 regulates CD₈+ T cell subpopulations and affects tumor immunity.
CTNNB1 (β-Catenin) is involved in cell signaling as part of the Wnt signaling pathway. Proteins in this pathway attach to CTNNB1 and trigger protein migration to the nucleus. CTNNB1 is associated with desmoid tumors, pilomatricoma, Wilm's tumor, aldosterone-producing adenoma, ovarian cancer, and other cancers.
FGFR including FGFR1, FGFR3, or FGFR4 (and fusions), is a receptor tyrosine kinase amplified in numerous cancers including squamous NSCLC, breast, ovarian, bladder, gastric and endometrial
ROS1 is a proto-oncogene receptor tyrosine kinase highly expressed in a variety of tumor cells
MYD88 (myeloid differentiation primary response 88) provides instructions for making a protein involved with signaling in immune cells and its mutation is found in cancer cells
HER2 (human epidermal growth factor receptor 2) is a growth-promoting protein on the outside of breast cells. Even HER2-negative breast cancer cells have HER2, however those with above the normal levels of HER2 are called HER2-positive. HER2 is a very important gene in the treatment of breast cancer. Approximately 1 of every 5 breast cancers has extra copies of the HER2 gene which leads to growth of the cancer cells.
TBXT, which is a transcription factor overexpressed in multiple cancers and correlated with tumor grade and aggressiveness.
PTP4A3 (PRL3) is a protein tyrosine phosphatase IVA3 is a prenylated phosphatase that is involved with cell signaling and overexpression causes cell growth.
MET (including exon-14 skipping mutations), which is a receptor tyrosine kinase;
alternatively spliced MET receptor exhibits decreased ubiquitination and delayed downregulation, leading to prolonged activation of MET and MAP kinase, which can be transforming.
USP7 is a deubiquitinating enzyme involved in prostate cancer, lung cancer, brain cancer, colon cancer, breast cancer, and other cancers.
NRF2(NFE2L2) is a basic leucine zipper protein that regulates the expression of antioxidant proteins that are cytoprotective; mutation or activation can promote cancer.
SF3B1 is a gene involved in splicing of RNA units. SF3B1 is involved in RNA splicing, mRNA splicing minor pathway, and PKN1 activated stimulation of the Androgen Receptor. Mutations to SF3B1 have been related to various cancers.
Any of the Ikaros family of proteins (IKZF 1, 2, 3, 4, or 5). IKZF 2 (Helios) and IKZF 4 (Eos) are selectively expressed in Treg cells but not effector or memory cells. FoxP₃/IKZF4/CtBP1 forms an inhibitory complex that suppresses gene expression (IL-2, IFN-γ) in Tregs and maintains its suppressive signature. Knocking down IKZF4 in Tregs abrogates the cell's ability to suppress immune responses and enables partial effector function. IKZF2 regulates Treg differentiation through a distinct mechanism from IKZF4. IKZF2 knockout in FoxP3-expressing Tregs promotes loss of inhibitory properties (with an increase in IL-2) and expression of T-effector cytokines via STAT5 (which regulates FoxP3).
MEN1 is a putative tumor suppressor associated with multiple endocrine neoplasia type 1 (MEN-1 syndrome). MEN1 is an autosomal dominant disorder in which affected individuals variably develop tumors in the parathyroids, anterior pituitary, and enteropancreatic endocrine tissue.
JCV proteins are encoded by the JC virus genome. In people with weakened immune systems, the JC virus can cause a serious brain infection called progressive multifocal leukoencephalopathy (PML). PML damages the outer coating of your nerve cells. It can cause permanent disabilities and can even be deadly. The JC virus genome encodes large and small tumor-antigens, the agnoprotein, and capsid proteins VP1 to VP3. The capsid proteins play a role in cellular entry and the agnoprotein plays a role in virion maturation.
CYP17A1 and CYP20A1 are heme proteins and are members of the cytochromes P450 family. The cytochrome P450 proteins are monooxygenases that catalyze many reactions involved in drug metabolism and synthesis of cholesterol, steroids, and other lipids. Many P450s are important enzymes for drug metabolism and other P450s play physiological roles by metabolizing endogenous substrates. For example, CYP17A1 is largely associated with endocrine effects and steroid hormone metabolism and mutations are associated with rare forms of congenital adrenal hyperplasia, specifically 17α-hydroxylase deficiency/17,20-lyase deficiency and isolated 17,20-lyase deficiency. CYP20A1 is an orphan isoform in humans that is expressed in the brain and liver.
BKV proteins are encoded by the BK virus genome. The human polyomavirus BK (BKV) infects humans worldwide and establishes a persistent infection in the kidney. The BK virus genome encodes three regulatory proteins, large and small tumor-antigen and the agnoprotein, as well as the capsid proteins VP1 to VP3. The agnoprotein helps regulate the virus replication and disrupt host cell processes once the viruses enter cells.
MEK1/2 are extracellular signal-regulated kinases that participate in the Ras/Raf/MEK/ERK pathway, a signaling cascade that regulates various cellular processes such as proliferation, differentiation, and cell cycle progression in response to a variety of extracellular signals. Overactivation or mutations in this pathway is linked to many cancers and the inhibition of MEK blocks cell proliferation leading to apoptosis. For example, β3-αC loop MEK1 mutants exhibit a strong oncogenic potential, but differential sensitivity to MEK inhibitors in clinic therapy or trials.
Ataxin-2 is a member of the Like-Sm (LSm) protein family and participates in a large number of functions related to RNA processing and RNA metabolism. Mutations in ATXN2 cause the neurodegenerative disease spinocerebellar ataxia type 2 (SCA2).
JAK2 is a non-receptor tyrosine kinase and a member of the Janus kinase family. It has been implicated in signaling by members of the type II cytokine receptor family, the GM-CSF receptor family, the gp130 receptor family, and the single chain receptors. JAK2 gene fusions with the TEL(ETV6) (TEL-JAK2) and PCM1 genes have been found in patients suffering leukemia and mutations in JAK2 have been implicated in polycythemia vera, essential thrombocythemia, and myelofibrosis as well as other myeloproliferative disorders.
PTPN11(SHP2) is a non-receptor tyrosine phosphatase that serves as a mediator of RTK-signaling. Overexpression has been observed in cancers with recurrent mutations observed in AML, JMML, and neuroblastoma; loss or inhibition of SHP2 has been shown to suppress proliferation of AML or other RTK-driven cancers
ERK1/ERK2 are extracellular signal-regulated kinases that participate in the Ras/Raf/MEK/ERK pathway, a signaling cascade that regulates various cellular processes such as proliferation, differentiation, and cell cycle progression in response to a variety of extracellular signals. Overactivation of this pathway is linked to many cancers.
BRAF Type II mutants are BRAF mutations classified as “constitutive active RAS-independent dimers with high or intermediate BRAF kinase activity involving codons outside 600, including BRAF fusion mutants.” Patients with Type II mutants typically have shorter survival times than those with Type I mutants and the cancer can be more aggressive.
ERBB3 is a transmembrane pseudo-RTK with strong genetic links to cancer.
GRB2 is a scaffold adapter involved in RTK signaling to downstream pathways including MAPK. GRB2 recruits to a variety of signaling molecules to receptors to form multimeric signaling complexes that lead to cellular responses such as proliferation and invasion, and is therefore linked to cancer and tumorigenesis.
CBP (CREBBP) is a transcriptional coactivator involved in the transcriptional coactivation of many different transcription factors and are therefore involved in a wide array of cellular activities, such as DNA repair, cell growth, differentiation and apoptosis. It is associated with a range of cancers including leukemia, NSCLC, HCV-associated hepatocellular carcinoma, melanoma, lung, lymphoma and bladder.
ATAD2 is a bromo/ATP helicase which can be a transcriptional co-activator of the nuclear receptor ESR1 required to induce a subset of estradiol target genes and can play a role in triple negative breast cancer
BAP1 is a deubiquitinating enzyme that can function as a tumor suppressor and a metastasis suppressor in cancer. BAP1's ability to regulate gene environment interactions in carcinogenesis has been linked to its dual role in the nucleus and in the cytoplasm. In the nucleus, BAP1 modulates the transcriptional regulation of several gene programs and promotes DNA repair by facilitating homologous recombination. It is found in PBRM1-deficient CRC.
BRPF1 is a bromodomain containing histone reader that associates with Moz and Morf, bearing HAT activity for H3. BRPF1 plays a role in cancer such as hematopoietic cancer including leukemia.
BRD4 is an epigenetic reader and a member of the BET protein family. BRD4 binds acetylated histones and plays a central role in controlling cellular gene transcription and proliferation and is therefore important in angiogenesis and the development of inflammation-associated diseases, cardiovascular diseases, central nervous system disorders and cancers.
EPAS1(HIF2a) belongs to a group of transcription factors involved in the physiological response to oxygen concentration and is encoded under hypoxic conditions. It is also important in the development of the heart, and for maintaining the catecholamine balance required for protection of the heart. Mutation often leads to neuroendocrine tumors, such as such as paragangliomas, somatostatinomas and/or pheochromocytomas.
KMT2D is a histone methyltransferase with a strong genetic link to cancer. The protein co-localizes with lineage determining transcription factors on transcriptional enhancers and is essential for cell differentiation and embryonic development. It also plays critical roles in regulating cell fate transition, metabolism, and tumor suppression.
Menin is a scaffolding protein that binds in a bidentate fashion to N-terminus of KMT2A (MLL) and MLL-fusion proteins, enabling binding and localization to chromatin; linked to leukemia and other cancers
MLLT1(ENL) is a YEATS domain containing protein; plays a role in transcriptional initiation/elongation (YEATS domain dependent) and a key interactor with DOTiL
DOT1L is a histone H3K79 methyltransferase that methylates lysine 79 on histone H3, an evolutionarily conserved methylation mark. DOT1L is involved in a number of key processes ranging from gene expression to DNA-damage response and cell cycle progression. DOT1L has also been implicated in the development of mixed lineage leukemia (MLL)-rearranged leukemia.
NSD2 is a histone methyltransferase that is expressed ubiquitously in early development and overexpressed in cancer cells, including in ALL, CLL and MCL.
TAU are the six highly soluble protein isoforms produced by alternative splicing from the gene MAPT (microtubule-associated protein tau). TAU proteins have roles primarily in maintaining the stability of microtubules in axons and are abundant in the neurons of the central nervous system (CNS). Pathologies and dementias of the nervous system such as Alzheimer's disease and Parkinson's disease are associated with tau proteins that have become hyperphosphorylated insoluble aggregates called neurofibrillary tangles.
HTT is the Huntington protein. HTT is essential for development and is highly expressed in neurons and testes. Huntingtin upregulates the expression of Brain Derived Neurotrophic Factor (BDNF) at the transcription level, and its mutated form leads to Huntington Disease.
NSD3 is a histone methyltransferase and a driver of 8p11-12 amplification found in cancers including squamous lung cancer, breast cancer and AML
SNCA is a member of the synuclein family, which is involved in regulation of dopamine release and transport, fibrillization of microtubule associated protein tau, and neuroprotective phenotype in non-dopaminergic neurons. Mutation of SNCA is related to neurodegenerative diseases, such as Parkinson's disease, Alzheimer's disease (AD), Lewy bodies' disease (LBD) and Muscular System Atrophy (MSA).
SMARCA2 and SMARCA4 are proteins encoded by the SWI/SNF family of proteins that have helicase and ATPase activities and regulate transcription of genes by altering chromatin structure as an ATP-dependent chromatin remodeler
BTK is a tyrosine kinase that plays a crucial role in the oncogenic signaling that is critical for proliferation and survival of leukemic cells in many B cell malignancies. BTK was initially shown to be mutated in the primary immunodeficiency X-linked agammaglobulinemia (XLA) and is essential at various stages of B lymphocyte development.
TAF1 is a TBP-associated factor with kinase domains, acetyltransferase and bromodomains. TAF1 is an important component of the transcription factor II D complex that serves a vital function during transcription initiation. Variants of the TAF1 gene have been associated with neurodevelopmental disorders, including intellectual disabilities.
IRAK4 is a threonine/serine protein kinase involved in signaling innate immune responses from Toll-like receptors (TLR). The loss of IRAK4 or its intrinsic kinase activity can entirely stop signaling through the TLR pathways. It is therefore relevant for various inflammatory disorders including rheumatoid arthritis, inflammatory bowel disease and other autoimmune diseases.
SARM1 is a negative regulator of Toll-Like Receptor-activated transcriptional programs.

After axon injury, SARM1 initiates a “self-destruct” mechanism to degrade the metabolite NAD+. This results in metabolic failure in neurons, leading to axon degeneration.
PPM1D (WIP1) is an oncoprotein and a member of the PP2C family of Ser/Thr protein phosphatases. PPM1D is a negative regulator of the cell stress response pathway and is amplified in various cancers, including breast, esophageal, colon, hematological, thyroid, sarcomas, lung, an ovarian.

In some embodiments, a coronavirus protein is degraded. In some embodiments, the coronavirus protein is a beta coronavirus protein. In some embodiments, the coronavirus protein is a Severe Acute Respiratory Syndrome (SARS)-CoV protein, a Middle Eastern Respiratory Syndrome (MERS)-CoV protein, or a SARS-CoV-2 protein. In some embodiments, the Target Protein is a SARS-CoV-2 protein. In some embodiments, the SARS-CoV2 protein is selected from a structural protein selected from a spike (S) protein (Accession #BCA87361.1), a membrane (M) protein (Accession #BCA87364.1), an envelope (E) protein (Accession #BCA87363.1), or a nucleocapsid phosphoprotein (N) protein (Accession #BCA87368.1), or a sequence at least 70%, 75%, 80%, 85%, 90%, 95%, or 98% homologous thereto, or a homolog, mutant, conjugate, derivative, fragment, or ortholog thereof. In some embodiments, the SARS-CoV2 protein is a non-structural protein, including nsp1 (leader protein) (Accession #YP_009725297.1), nsp2 (Accession #YP_009725298.1), nsp3 (papain-like proteinase) (Accession #YP_009725299.1), nsp4 (Accession #YP_009725300.1), nsp5 (3C-like proteinase) (Accession #YP_009725301.1), nsp6 (putative transmembrane domain) (Accession #YP_009725302.1), nsp7 (Accession #YP_009725303.1), nsp8 (primase) (Accession #YP_009725304.1), nsp9 (Accession #YP_009725305.1), nsp10 (Accession #YP_009725306.1), nsp11 (Accession #YP_009725312.1), nsp12 (RNA dependent RNA polymerase) (Accession #YP_009725307.1), nsp13 (helicase) (Accession #YP_009725308.1), nsp14 (3′-5′ exonuclease, guanine N7-methyltransferase) (Accession #YP_009725309.1), nsp15 (endoRNAse) (Accession #YP_009725310.1), or nsp16 (2′-O-ribose-methyltransferase) (Accession #YP_009725311.1), or a sequence at least 70%, 75%, 80%, 85%, 90%, 95%, or 98% homologous thereto, or a homolog, mutant, conjugate, derivative, fragment, or ortholog thereof. In some embodiments, the SARS-CoV2 protein is selected from ORF3a protein (Accession #BCA87362.1), ORF6 protein (accessory protein 6) (Accession #BCA87365.1), ORF7a protein (accessory protein 7a)(Accession #BCA87366.1), ORF7b protein (accessory protein 7b) (Accession #BCB15096.1), ORF8 protein (Accession #QJA17759.1), ORF9b protein (accessory protein 9b) (UniprotKB-PODTD2), or ORF10 protein (Accession #BCA87369.1), or a sequence at least 70%, 75%, 80%, 85%, 90%, 95%, or 98% homologous thereto, or a homolog, mutant, conjugate, derivative, fragment, or ortholog thereof. In some embodiments, the SARS-CoV2 protein is ORF3b protein (see Konno et al., SARS-CoV-2 ORF3b Is a Potent Interferon Antagonist Whose Activity Is Increased by a Naturally Occurring Elongation Variant. Cell Reports, Volume 32, Issue 12, 22 Sep. 2020, 108185) encoded by nucleotides 25814-25880 of NCBI Reference Sequence: NC_045512.2, or a sequence at least 70%, 75%, 80%, 85%, 90%, 95%, or 98% homologous thereto, or a homolog, mutant, conjugate, derivative, fragment, or ortholog thereof.

In other embodiments, Target Protein is a viral protein of a virus other than coronavirus, for example a protease, polymerase, exonuclease, helicase, glycosyltransferase, esterase, integrase, reverse transcriptase, kinase, primase, proteinase, methyltransferase, or nucleotidase.

In certain embodiments a compound of the present invention is used to treat a coronavirus variant for example a SARS-CoV-2 variants selected from alpha, beta, gamma, delta, epsilon, eta, iota, kappa, mu, and zeta. Non limiting examples of SARS-CoV-2 alpha variants include B.1.1.7 and Q.1-Q.8. Non limiting examples of SARS-CoV-2 beta variants include B.1.351, B.1.351.2, and B.1.351.3. Non limiting examples of SARS-CoV-2 gamma variants include P.1, P.1.1, and P.1.2. Non limiting examples of SARS-CoV-2 delta variants include B.1.617.2 and AY.1. Non limiting examples of SARS-CoV-2 epsilon variants include B.1.427 and B.1.429. Non limiting examples of SARS-CoV-2 eta variants include B.1.525. Non limiting examples of SARS-CoV-2 iota variants include B.1.526. Non limiting examples of SARS-CoV-2 kappa variants include B.1.617.1. Non limiting examples of SARS-CoV-2 mu variants include B.1.621 and B.1.621.1. Non limiting examples of SARS-CoV-2 zeta variants include P.2.

In certain embodiments a compound of the present invention is used to treat a coronavirus other than SARS-CoV-2. Additional examples of coronaviruses include: Severe Acute Respiratory Syndrome coronavirus (SARS-CoV), Bat SARS-like coronavirus WIV1 (Bat SL-CoV-WIVI), alpha coronaviruses 229E (HCoV-229E), New Haven coronavirus NL63 (HCoV-NL63), beta coronaviruses OC43 (HCoV-OC43), coronavirus HKIJ I (HCoV-HKU 1), and Middle East Respiratory Syndrome coronavirus (MERS-CoV).

It has been reported that certain proteins with a β-hairpin turn containing a glycine at a key position (a “g-loop protein” or “g-loop degron”) acts as a “structural degron” for cereblon when the cereblon is also bound to a thalidomide-like molecule (IMiD) neosubstrate protein. Such “g-loop degron” containing proteins generally include a small anti-parallel p-sheet forming a 0-hairpin with an α-turn, with a geometric arrangement of three backbone hydrogen bond acceptors at the apex of a turn (positions i, i+1, and i+2), with a glycine residue at a key position (i+3) (see, e.g., Matyskiela, et al, A novel cereblon modulator recruits GSPT1 to the CRL4-CRBN ubiquitin ligase. Nature 535, 252-257 (2016); Sievers et al., Defining the human C2H2 zinc finger degrome targeted by thalidomide analogs through CRBN. Science 362, eaat0572 (2018)). These g-loop degrons have been identified in a number of proteins, including, but not limited to, Sal-like 4 (SALL4), GSPT1, IKFZ1, IKFZ3, and CK1α, ZFP91, ZNF93, etc.

In some embodiments, a tricyclic heterobifunctional compound of the present invention or pharmaceutical salt thereof, optionally in a pharmaceutical composition as described herein can be administered in an effective amount to a host to degrade a protein containing a g-loop degron, wherein the protein is selected from a protein kinase, C2H2 containing zinc finger protein, an RNA-recognition motif containing protein, a zinc beta ribbon containing protein, a beta-propeller containing protein, a P-loop NTPase containing protein, a really interesting new gene (RING)-finger domain containing protein, an SRC Homology 3 (SH3)-domain containing protein, an immunoglobulin E-set domain containing protein, a Tudor-domain containing protein, a zinc finger FYVE/PHD-type containing protein, an Ig-like domain containing protein, a ubiquitin-like domain containing protein, a concanavalin-like domain containing protein, a C1-domain containing protein, a Pleckstrin homology (PH)-domain containing protein, an GB-fold-domain containing protein, an NADP Rossman-fold-domain containing protein, an Actin-like ATPase domain containing protein, and a helix-turn-helix (HTH)-domain containing protein. In some embodiments, the protein kinase, C2H2 containing zinc finger protein, an RNA-recognition motif containing protein, a zinc beta ribbon containing protein, a beta-propeller containing protein, a P-loop NTPase containing protein, a really interesting new gene (RING)-finger domain containing protein, an SRC Homology 3 (SH3)-domain containing protein, an immunoglobulin E-set domain containing protein, a Tudor-domain containing protein, a zinc finger FYVE/PHD-type containing protein, an Ig-like domain containing protein, a ubiquitin-like domain containing protein, a concanavalin-like domain containing protein, a C₁-domain containing protein, a Pleckstrin homology (PH)-domain containing protein, an GB-fold-domain containing protein, an NADP Rossman-fold-domain containing protein, an Actin-like ATPase domain containing protein, or a helix-turn-helix (HTH)-domain containing protein is overexpressed or contains a gain-of-function mutation. In some embodiments, the degron is stabilized by internal hydrogen bonds from an ASX motif and a ST motif.

In some embodiments, a tricyclic heterobifunctional compound of the present invention or pharmaceutical salt thereof, optionally in a pharmaceutical composition as described herein can be administered in an effective amount to a host to degrade a protein with a “g-loop degron,” wherein the “g-loop degron” comprises a [D/N]XX[S/T]G motif, wherein D=aspartic acid, N=asparagine, X can be any amino acid residue, S=serine, T=threonine, and G=glycine. In certain embodiments, the “g-loop degron” containing protein comprises an amino acid sequence of DXXSG, wherein D=aspartic acid, X can be any amino acid residue, S=serine, and G=glycine. In another embodiment, the “g-loop degron” containing protein comprises an amino acid sequence of NXXSG, wherein N=asparagine, X can be any amino acid residue, S=serine, and G=glycine. In yet another embodiment, the “g-loop degron” containing protein comprises an amino acid sequence of DXXTG, wherein D=aspartic acid, X can be any amino acid residue, T=threonine, and G=glycine. In still another embodiment, “g-loop degron” containing protein comprises an amino acid sequence of NXXTG, wherein N=asparagine, X can be any amino acid residue, T=threonine, and G=glycine. In some embodiments, the “g-loop degron” containing protein comprises an amino acid sequence of CXXCG, wherein C=cysteine, X can be any amino acid residue, and G=glycine. In certain embodiments, the “g-loop degron” containing protein comprises an amino acid sequence of NXXNG, wherein N=asparagine, X can be any amino acid residue, and G=glycine.

In some embodiments, a tricyclic heterobifunctional compound of the present invention or pharmaceutical salt thereof, optionally in a pharmaceutical composition as described herein can be administered in an effective amount to a host to degrade a protein with a C2H2 zinc-finger domain containing a “g-loop degron”. In some embodiments, the zinc-finger domain has the consensus sequence C-X-X-C-G, wherein C=cysteine, X=any amino acid, and G=glycine. In an alternative embodiment, the protein with a zinc-finger domain has the consensus sequence Q-C-X-X-C-G (SEQ ID NO: 1), wherein C=cysteine, X=any amino acid, G=glycine, and Q=glutamine. In a still further embodiment, the zinc-finger domain has the consensus sequence Q-C-X₂-C-G-X₃-F-X₅-L-X₂-H-X₃-H (SEQ ID NO: 2), wherein C=cysteine, X=any amino acid, G=glycine, Q=glutamine, F=phenylalanine, L=leucine, and H=histidine. In some embodiments, the C2H2 zinc-finger domain containing X₂-C-X₂-CG-X₂-C-X₅ (SEQ ID NO: 3), wherein C=cysteine, X=any amino acid, and G=glycine. In some embodiments, the C₂H₂ zinc-finger domain containing protein is over-expressed. In some embodiments, the expression of C2H2 zinc-finger containing protein is associated with a disease or disorder, including, but not limited to, cancer.

For example, a heterobifunctional compound of the present invention, or pharmaceutical salt thereof, optionally in a pharmaceutical composition as described herein is administered to a host to degrade Zinc Finger Protein, Atypical E3 Ubiquitin Ligase (ZFP91). Zinc Finger Protein, Atypical E3 Ubiquitin Ligase contains a Cys2-His2 zinc finger, and protects tumor cell survival and confers chemoresistance through forkhead box A1 (FOXA1) destabilization (see, e.g., Tang, et al. The ubiquitanse ZFP91 promotes tumor cell survival and confers chemoresistance through FOXA1 destabilization, Carcinogenesis, Col. 41(1), January 2020). Zinc Finger Protein, Atypical E3 Ubiquitin Ligase is believed to act through noncanonical NF-κB pathway regulation, and its overexpression leads to increased NF-κB signaling pathway activation has been implicated in a number of cancers, including gastric cancer, breast cancer, colon cancer, kidney cancer, ovarian cancer, pancreatic cancer, stomach cancer, prostate cancer, sarcoma, and melanoma (see, e.g., Paschke, ZFP91 zinc finger protein expression pattern in normal tissues and cancers. Oncol Lett. 2019; March; 17(3):3599-3606). In certain embodiments, a compound of the present invention, or pharmaceutical salt thereof, optionally in a pharmaceutical composition as described herein is used to degrade Zinc Finger Protein, Atypical E3 Ubiquitin Ligase for the treatment of a cancer, including but not limited to, gastric cancer, breast cancer, colon cancer, kidney cancer, ovarian cancer, pancreatic cancer, stomach cancer, prostate cancer, sarcoma, and melanoma. In certain embodiments, a compound of the present invention, or pharmaceutical salt thereof, optionally in a pharmaceutical composition as described herein is used to degrade Zinc Finger Protein, Atypical E3 Ubiquitin Ligase for the treatment of a sarcoma, melanoma, or gastric cancer.

In another embodiment, a tricyclic heterobifunctional compound of the present invention, or pharmaceutical salt thereof, optionally in a pharmaceutical composition as described herein is administered to a host to degrade zinc finger protein 276 (ZFP276).

In yet another embodiment, a tricyclic heterobifunctional compound of the present invention, or pharmaceutical salt thereof, optionally in a pharmaceutical composition as described herein is administered to a host to degrade Zinc finger protein 653 (ZFP653). Zinc finger protein 653 may act as a more general repressor of transcription by competition with GRIP1 and other p160 coactivators for binding to SF1 (see, e.g., Borud et al., Cloning and characterization of a novel zinc finger protein that modulates the transcriptional activity of nuclear receptors. Molec. Endocr. 17: 2303-2319, 2003).

As other examples, a tricyclic heterobifunctional compound of the present invention, or pharmaceutical salt thereof, optionally in a pharmaceutical composition as described herein is administered to a host in an effective amount to degrade Zinc finger protein 692 (ZFP692). Zinc finger protein 692, also known as AICAR response element binding protein (AREBP), contains a Cys2-His2 zinc finger, and is believed to be a key modulator of hepatic glucose production regulated by AMPK in vivo (See Shirai et al., AICAR response element binding protein (AREBP), a key modulator of hepatic glucose production regulated by AMPK in vivo. Biochem Biophys Res Commun. 2011 Oct. 22; 414(2):287-91). The overexpression of and its overexpression has been associated with the promotion of colon adenocarcinoma and metastasis by activating the PI3K/AKT pathway (see, for example, Xing et al., Zinc finger protein 692 promotes colon adenocarcinoma cell growth and metastasis by activating the PI3K/AKT pathway. Int J Oncol. 2019 May; 54(5): 1691-1703), and the development of metastasis in lung adenocarcinomas and lung carcinoma. Knockdown of Zinc finger protein 692 expression via short interfering RNA reduced cell invasion and increased apoptosis in lung carcinoma cells and suppressed lung carcinoma tumor growth in a xenograft model (see, e.g., Zhang et al., ZNF692 promotes proliferation and cell mobility in lung adenocarcinoma. Biochem Biophys Res Commun. 2017 Sep. 2; 490(4):1189-1196). Accordingly, in certain embodiments, a compound of the present invention, or pharmaceutical salt thereof, optionally in a pharmaceutical composition as described herein is used to degrade Zinc finger protein 692 for the treatment of a lung or colon cancer, including a lung adenocarcinoma or carcinoma or a colon adenocarcinoma.

A tricyclic heterobifunctional compound of the present invention, or pharmaceutical salt thereof, optionally in a pharmaceutical composition as described herein can also administered in an effective amount to a host to degrade Zinc finger protein 827 (ZFP827). Zinc finger protein 827 is a zinc finger protein that regulates alternative lengthening of telomeres (ALT) pathway by binding nuclear receptors and recruiting the nucleosome remodeling and histone deacetylation (NURD) complex to telomeres to induce homologous recombination (see, e.g., Conomos, D., Reddel, R. R., Pickett, H. A. NuRD-ZNF827 recruitment to telomeres creates a molecular scaffold for homologous recombination. Nature Struct. Molec. Biol. 21: 760-770, 2014). Zinc finger protein 827 has been associated with ALT-associated promyelocytic leukemia (PML) nuclear bodies (APBs) and other telomeric aberrations. Accordingly, in certain embodiments, a compound of the present invention, or pharmaceutical salt thereof, optionally in a pharmaceutical composition as described herein is used to degrade ZNF827 in ALT-associated disorders, including, but not limited to ALT-positive promyelocytic leukemia, osteosarcoma, adrenal/PNS neuroblastoma, breast cancer, glioblastoma, colorectal cancer, pancreatic neuroendocrine tumor (NET), neuroendocrine tumor, colorectal cancer, liver cancer, soft tissue cancers, including leiomyosarcoma, malignant fibrous histiocytoma, liposarcoma, stomach/gastric cancer, testicular cancer, and thyroid cancer.

In other embodiments, a tricyclic heterobifunctional compound of the present invention, or pharmaceutical salt thereof, optionally in a pharmaceutical composition as described herein is administered in an effective amount to a host to degrade E4F Transcription Factor 1 protein (E4F1). E4F Transcription Factor 1 is believed to function as a ubiquitin ligase for p53, and is a key posttranslational regulator of p53 that plays an important role in the cellular life-or-death decision controlled by p53 (see, e.g., Le Cam et al., The E4F protein is required for mitotic progression during embryonic cell cycles. Molec. Cell. Biol. 24: 6467-6475, 2004). E4F1 overexpression has been associated with the development of myeloid leukemia cells (see, e.g., Hatachi et al., E4F1 deficiency results in oxidative stress-mediated cell death of leukemic cells. J Exp Med. 2011 Jul. 4; 208(7): 1403-1417). Accordingly, in certain embodiments, a compound of the present invention, or pharmaceutical salt thereof, optionally in a pharmaceutical composition as described herein is used to degrade E4F Transcription Factor 1 for the treatment of a leukemia of myelogenous origin, including but not limited to, acute myelogenous leukemia (AML), undifferentiated AML, myeloblastic leukemia with minimal cell maturation, myeloblastic leukemia with cell maturation, promyelocytic leukemia, myelomonocytic leukemia, myelomonocytic leukemia with eosinophilia, monocytic leukemia, erythroleukemia, megakaryoblastic leukemia, chronic myelogenous leukemia (CML), juvenile myelomonocytic leukemia (JMML), chronic myelomonocytic leukemia (CMML), a myeloproliferative neoplasm, including for example, polycythemia vera (PV), essential thrombocythemia (ET), myeloid metaplasia with myelofibrosis (MMM), hypereosinophilic syndrome (HES), systemic mast cell disease (SMCD), myelofibrosis, and primary myelofibrosis. E4F1 expression is also essential for survival in p53-deficient cancer cells (see, e.g., Rodier et al., The Transcription Factor E4F1 Coordinates CHK1-Dependent Checkpoint and Mitochondrial Functions. Cell Reports Volume 11, ISSUE 2, P220-233, Apr. 14, 2015). Accordingly, in certain embodiments, a compound of the present invention, or pharmaceutical salt thereof, optionally in a pharmaceutical composition as described herein is used to degrade E4F Transcription Factor 1 for the treatment of a p53-deficient associated disorder, including, but not limited to ovarian cancer, small cell lung cancer, pancreatic cancer, head and neck squamous cell carcinoma, and triple negative breast cancer.

In another aspect, a tricyclic heterobifunctional compound of the present invention, or pharmaceutical salt thereof, optionally in a pharmaceutical composition as described herein is administered in an effective amount to a host to degrade Zinc finger protein 517 (ZFP517). Zinc finger protein 517 has been identified as an oncogenic driver in adrenocortical carcinoma (ACC) (see, e.g., Rahane et al., Establishing a human adrenocortical carcinoma (ACC)-specific gene mutation signature. Cancer Genet. 2019; 230:1-12). Accordingly, in certain embodiments, a compound of the present invention, or pharmaceutical salt thereof, optionally in a pharmaceutical composition as described herein is used to Zinc finger protein 517 for the treatment of adrenocortical carcinoma.

In yet another aspect, a tricyclic heterobifunctional compound of the present invention, or pharmaceutical salt thereof, optionally in a pharmaceutical composition as described herein is administered in an effective amount to a host to degrade Zinc finger protein 582 (ZFP582). Zinc finger protein 582 is believed to be involved in n DNA damage response, proliferation, cell cycle control, and neoplastic transformation, most notably cervical, esophageal, and colorectal cancer (see, e.g., Huang et al., Methylomic analysis identifies frequent DNA methylation of zinc finger protein 582 (ZNF582) in cervical neoplasms. PLoS One 7: e41060, 2012; Tang et al., Aberrant DNA methylation of PAX1, SOX1 and ZNF582 genes as potential biomarkers for esophageal squamous cell carcinoma. Biomedicine & Pharmacotherapy Volume 120, December 2019, 109488; Harada et al., Analysis of DNA Methylation in Bowel Lavage Fluid for Detection of Colorectal Cancer. Cancer Prev Res; 7(10); 1002-10; 2014). Accordingly, in certain embodiments, a compound of the present invention, or pharmaceutical salt thereof, optionally in a pharmaceutical composition as described herein is used to degrade Zinc finger protein 582 for the treatment of a cancer, including but not limited to cervical cancer, including cervical adenocarcinoma, esophageal cancer, including squamous cell carcinoma and adenocarcinoma, and colorectal cancer.

In another embodiment, a tricyclic heterobifunctional compound of the present invention, or pharmaceutical salt thereof, optionally in a pharmaceutical composition as described herein is administered in an effective amount to a host to degrade Zinc finger protein 654 (ZFP654).

Alternatively, a tricyclic heterobifunctional compound of the present invention, or pharmaceutical salt thereof, optionally in a pharmaceutical composition as described herein is administered in an effective amount to a host to degrade Zinc finger protein 787 (ZFP787).

A tricyclic heterobifunctional compound of the present invention, or pharmaceutical salt thereof, optionally in a pharmaceutical composition as described herein can be administered in an effective amount to a host to degrade Hypermethylated in Cancer 1 (HIC1) protein. Hypermethylated in Cancer 1 protein contains an N-terminal BTB/POZ protein-protein interaction domain and 5 Kruppel-like C2H2 zinc finger motifs in its C-terminal half (see, e.g., Deltour et al., The carboxy-terminal end of the candidate tumor suppressor gene HIC-1 is phylogenetically conserved. Biochim. Biophys. Acta 1443: 230-232, 1998). Expression of Hypermethylated in Cancer 1 protein gene disorder Miller-Dieker syndrome (see, e.g., Grimm et al., Isolation and embryonic expression of the novel mouse gene Hicd, the homologue of HIC1, a candidate gene for the Miller-Dieker syndrome. Hum. Molec. Genet. 8: 697-710, 1999).

A tricyclic heterobifunctional compound of the present invention, or pharmaceutical salt thereof, optionally in a pharmaceutical composition as described herein is administered in an effective amount to a host to degrade Hypermethylated in Cancer 2 (HIC2) protein.

A tricyclic heterobifunctional compound of the present invention, or pharmaceutical salt thereof, optionally in a pharmaceutical composition as described herein can be administered in an effective amount to a host to degrade GDNF-Inducible Zinc Finger Protein 1 (GZF1). GDNF-Inducible Zinc Finger Protein 1 is a transcriptional regulator that binds to a 12-bp GZF1 response element (GRE) and represses gene transcription (see, e.g., Morinaga et al., GDNF-inducible zinc finger protein 1 is a sequence-specific transcriptional repressor that binds to the HOXA10 gene regulatory region. Nucleic Acids Res. 33: 4191-4201, 2005).

Alternatively, for example, a tricyclic heterobifunctional compound of the present invention, or pharmaceutical salt thereof, optionally in a pharmaceutical composition as described herein can be administered in an effective amount to a host to degrade Odd Skipped Related 1 (OSR1) protein. Odd Skipped Related 1 protein contains 3 C2H2-type zinc fingers, a tyrosine phosphorylation site, and several putative PXXP SH3 binding motifs (see, e.g., Katoh, M. Molecular cloning and characterization of OSR1 on human chromosome 2p24. Int. J. Molec. Med. 10: 221-225, 2002).

In another aspect, a tricyclic heterobifunctional compound of the present invention, or pharmaceutical salt thereof, optionally in a pharmaceutical composition as described herein is administered in an effective amount to a host to degrade Odd Skipped Related 2 (OSR2) protein.

In yet another embodiment, a selected tricyclic heterobifunctional compound of the present invention, or pharmaceutical salt thereof, optionally in a pharmaceutical composition as described herein can be administered to a host in an effective amount to degrade SAL-Like 4 (SALL4) protein. SAL-Like 4 protein has 3 C2H2 double zinc finger domains of the SAL-type, the second of which has a single C2H2 zinc finger attached at its C-terminal end, as well as an N-terminal C2HC zinc finger motif typical for vertebrate SAL-like proteins. SAL-Like 4 protein mutations are associated with the development of Duane-radial ray syndrome (see, e.g., Borozdin et al., SALL4 deletions are a common cause of Okihiro and acro-renal-ocular syndromes and confirm haploinsufficiency as the pathogenic mechanism. J. Med. Genet. 41: e113, 2004). SAL-Like 4 protein overexpression is associated with the promotion, growth and metastasis of a number of cancers, including lung cancer, gastric cancer, liver cancer, renal cancer, myelodysplastic syndrome, germ cell-sex cord-stromal tumors including dysgerminoma, yolk sac tumor, and choriocarcinoma, and leukemia, among others. Accordingly, in certain embodiments, a compound of the present invention, or pharmaceutical salt thereof, optionally in a pharmaceutical composition as described herein is used to degrade SAL-Like 4 protein for the treatment of a cancer, including but not limited to, gastric cancer, liver cancer, renal cancer, myelodysplastic syndrome, germ cell-sex cord-stromal tumors including dysgerminoma, yolk sac tumor, and choriocarcinoma, and leukemia, among others.

A selected tricyclic heterobifunctional compound of the present invention, or pharmaceutical salt thereof, optionally in a pharmaceutical composition as described herein can also be administered in an effective amount to a host to degrade B-Cell Lymphoma 6 (BCL6) protein. B-Cell Lymphoma 6 contains an autonomous transrepressor domain, and 2 noncontiguous regions, including the POZ motif, mediate maximum transrepressive activity. Translocations of the B-Cell Lymphoma 6 gene translocations are associated with the development of myeloproliferative disorders such as non-Hodgkin lymphomas. B-Cell Lymphoma 6 overexpression prevents increase in reactive oxygen species and inhibits apoptosis induced by chemotherapeutic reagents in cancer cells (see, e.g., Tahara et al., Overexpression of B-cell lymphoma 6 alters gene expression profile in a myeloma cell line and is associated with decreased DNA damage response. Cancer Sci. 2017 August; 108(8):1556-1564; Cardenas et al., The expanding role of the BCL6 oncoprotein as a cancer therapeutic target. Clin Cancer Res. 2017 Feb. 15; 23(4): 885-893). Accordingly, in certain embodiments, a compound of the present invention, or pharmaceutical salt thereof, optionally in a pharmaceutical composition as described herein is used to degrade B-Cell Lymphoma 6 for the treatment of a cancer, including but not limited to a hematologic or solid tumor, for example, but not limited to a B-cell leukemia or lymphoma, for example, but not limited to diffuse large B-cell lymphomas (DLBCLs) and ABC-DLBCL subtypes, B-acute lymphoblastic leukemia, chronic myeloid leukemia, breast cancer and non-small cell lung cancer.

Further, a selected tricyclic heterobifunctional compound of the present invention, or pharmaceutical salt thereof, optionally in a pharmaceutical composition as described herein is administered in an effective amount to a host to degrade B-Cell Lymphoma 6B (BCL6B) protein. B-Cell Lymphoma 6B protein contains an N-terminal POZ domain and 5 C-terminal zinc finger motifs, and is believed to act as a transcriptional repressor (see, e.g., Okabe et al., BAZF, a novel Bcl6 homolog, functions as a transcriptional repressor. Molec. Cell. Biol. 18: 4235-4244, 1998). Overexpression of B-Cell Lymphoma 6B protein has been associated with the development of germ cell tumors (Ishii et al., FGF2 mediates mouse spermatogonia stem cell self-renewal via upregulation of Etv5and Bcl6b through MAP2K1 activation. Development 139, 1734-1743 (2012)). Accordingly, in certain embodiments, a compound of the present invention, or pharmaceutical salt thereof, optionally in a pharmaceutical composition as described herein is used to degrade B-Cell Lymphoma 6B for the treatment of a cancer, including but not limited to, a germ cell cancer including but not limited to germinoma, including dysgerminoma and seminoma, a teratoma, yolk sac tumor, and choriocarcinomas.

Alternatively, a selected tricyclic heterobifunctional compound of the present invention, or pharmaceutical salt thereof, optionally in a pharmaceutical composition as described herein can be administered in an effective amount to a host to degrade Early Growth Response 1 (EGR1) protein. Early Growth Response 1 protein directly controls transforming growth factor-beta-1 gene expression, and has been shown to be involved in the proliferation and survival of prostate cancer cells by regulating several target genes, including cyclin D2 (CCND2), p19(Ink4d), and Fas, as well as glioma cells (see, e.g., Virolle et al., Erg1 promotes growth and survival of prostate cancer cells: identification of novel Egr1 target genes. J. Biol. Chem. 278: 11802-11810, 2003; Chen et al., Inhibition of EGR1 inhibits glioma proliferation by targeting CCND1 promoter. Journal of Experimental & Clinical Cancer Research Volume 36, Article number: 186 (2017)). One mechanism used by Egr1 to confer resistance to apoptotic signals was the ability of Egr1 to inhibit Fas expression, leading to insensitivity to FasL. Accordingly, in certain embodiments, a compound of the present invention, or pharmaceutical salt thereof, optionally in a pharmaceutical composition as described herein is used to degrade Early Growth Response 1 protein for the treatment of a cancer, including but not limited to a prostate cancer or glioma including, but not limited to, pilocytic astrocytoma, diffuse astrocytoma, anaplastic astrocytoma, glioblastoma multiforme.

In yet another aspect, a selected tricyclic heterobifunctional compound of the present invention, or pharmaceutical salt thereof, optionally in a pharmaceutical composition as described herein can be administered in an effective amount to a host to degrade Early Growth Response 4 (EGR4) protein. Early Growth Response 4 protein contains 3 zinc fingers of the C2/H2 subtype near the carboxy terminus (see, e.g., Crosby et al., Neural-specific expression, genomic structure, and chromosomal localization of the gene encoding the zinc-finger transcription factor NGFI-C. Proc. Nat. Acad. Sci. 89: 4739-4743, 1992). Overexpression of Early Growth Response 4 protein has been associated with the development of cholangiocarcinoma (see, e.g., Gong et al., Gramicidin inhibits cholangiocarcinoma cell growth by suppressing EGR4. Artificial Cells, Nanomedicine, and Biotechnology, 48:1, 53-59 (2019)). Accordingly, in certain embodiments, a compound of the present invention, or pharmaceutical salt thereof, optionally in a pharmaceutical composition as described herein is used to degrade Early Growth Response 4 protein for the treatment of a cancer, including but not limited to cholangiocarcinoma.

In certain aspects, a selected tricyclic heterobifunctional compound of the present invention, or pharmaceutical salt thereof, optionally in a pharmaceutical composition as described herein can be administered in an effective amount to a host to degrade Sal-Like 1 (SALL1) protein.

In an alternative embodiment, a selected tricyclic heterobifunctional compound of the present invention, or pharmaceutical salt thereof, optionally in a pharmaceutical composition as described herein can be administered in an effective amount to a host to degrade Sal-Like 3 (SALL3) protein. The SALL3 protein contains 4 double zinc finger (DZF) domains, each of which contains sequences identical or closely related to the SAL box, a characteristic stretch of 8 amino acids within the second zinc finger motif.

In yet another embodiment, a selected tricyclic heterobifunctional compound of the present invention, or pharmaceutical salt thereof, optionally in a pharmaceutical composition as described herein can be administered in an effective amount to a host to degrade Tumor protein p63 (TP63). Tumor protein p63 overexpression has been associated with lung cancer development and poor prognosis, radiation resistance in oral cancers and head and neck cancers, squamous cell carcinoma of the skin (see, e.g., Massion et al., Significance of p63 amplification and overexpression in lung cancer development and prognosis. Cancer Res. 2003 Nov. 1; 63(21):7113-21; Moergel et al., Overexpression of p63 is associated with radiation resistance and prognosis in oral squamous cell carcinoma. Oral Oncol. 2010 September; 46(9):667-71). Accordingly, in certain embodiments, a compound of the present invention, or pharmaceutical salt thereof, optionally in a pharmaceutical composition as described herein is used to degrade Tumor protein p63 for the treatment of a cancer, including but not limited to non-small cell lung cancer, small cell lung cancer, head and neck cancer, and squamous cell carcinoma of the skin.

In yet another embodiment, a selected tricyclic heterobifunctional compound of the present invention, or pharmaceutical salt thereof, optionally in a pharmaceutical composition as described herein can be administered in an effective amount to a host to degrade Widely-Interspaced Zinc Finger-Containing (WIZ) Protein.

A selected tricyclic heterobifunctional compound of the present invention, or pharmaceutical salt thereof, optionally in a pharmaceutical composition as described herein can also be administered in an effective amount to a host to degrade Zinc Finger and BTB Domain Containing Protein 7A (ZBTB7A). Zinc Finger and BTB Domain Containing Protein 7A expression is associated with a number of cancers, including prostate cancer, non-small cell lung cancer, bladder, breast cancer, prostate, ovarian, oral squamous cell carcinoma, and hepatocellular carcinoma (see, e.g., Han et al., ZBTB7A Mediates the Transcriptional Repression Activity of the Androgen Receptor in Prostate Cancer. Cancer Res 2019; 79:5260-71; Molloy et al., ZBTB7A governs estrogen receptor alpha expression in breast cancer. Journal of Molecular Cell Biology, Volume 10, Issue 4, August 2018, Pages 273-284). Accordingly, in certain embodiments, a compound of the present invention, or pharmaceutical salt thereof, optionally in a pharmaceutical composition as described herein is used to degrade Zinc Finger and BTB Domain Containing Protein 7A for the treatment of a cancer, including but not limited to prostate cancer, non-small cell lung cancer, breast cancer, oral squamous cell carcinoma, prostate, ovarian, glioma, bladder, and hepatocellular carcinoma.

In other aspects, a selected tricyclic heterobifunctional compound of the present invention, or pharmaceutical salt thereof, optionally in a pharmaceutical composition as described herein can be administered in an effective amount to a host to degrade Zinc Finger and BTB Domain Containing Protein 7B (ZBTB7B). Zinc Finger and BTB Domain Containing Protein 7B expression has been associated with breast, prostate, urothelial, cervical, and colorectal cancers. Accordingly, in certain embodiments, a compound of the present invention, or pharmaceutical salt thereof, optionally in a pharmaceutical composition as described herein is used to degrade Zinc Finger and BTB Domain Containing Protein 7B for the treatment of a cancer, including but not limited to breast, prostate, urothelial, cervical, and colorectal cancers.

A selected tricyclic heterobifunctional compound of the present invention, or pharmaceutical salt thereof, optionally in a pharmaceutical composition as described herein can be administered in an effective amount to a host to degrade casein kinase I, alpha I (CK1α or CK1-alpha). CK1-alpha is a bifunctional regulator of NF-kappa-B (see, e.g., Bidere et al., Casein kinase 1-alpha governs antigen-receptor-induced NF-kappa-B activation and human lymphoma cell survival. Nature 458: 92-96, 2009). CK1-alpha dynamically associates with the CBM complex on T cell receptor engagement to participate in cytokine production and lymphocyte proliferation. However, CK1-alpha kinase activity has a contrasting role by subsequently promoting the phosphorylation and inactivation of CARMA1. CK1-alpha has thus a dual ‘gating’ function which first promotes and then terminates receptor-induced NF-kappa-B. ABC DLBCL cells required CK1-alpha for constitutive NF-kappa-B activity, indicating that CK1-alpha functions as a conditionally essential malignancy gene. Expression of CK1-alpha has been associated with myelodysplastic disease with depletion of 5q (del(5q) MDS (see, e.g., Kronke, et al., Lenalidomide induces ubiquitination and degradation of CK1-alpha in del(5q) MDS. Nature 523: 183-188, 2015), colorectal cancer, breast cancer, leukemia, multiple myeloma, lung cancer, diffuse large B cell lymphoma, non-small cell lung cancer, and pancreatic cancer, amongst others (see, e.g., Richter et al., CK1α overexpression correlates with poor survival in colorectal cancer. BMC Cancer. 2018; 18: 140; Jiang et al., Casein kinase 1α: biological mechanisms and theranostic potential. Cell Commun Signal. 2018; 16: 23). Accordingly, in some embodiments, a compound of the present invention, or pharmaceutical salt thereof, optionally in a pharmaceutical composition as described herein is used to degrade casein kinase I, alpha I for the treatment of a cancer, including but not limited to colorectal cancer, breast cancer, leukemia, multiple myeloma, lung cancer, diffuse large B cell lymphoma, non-small cell lung cancer, pancreatic cancer, myelodysplastic syndromes including but not limited to 5q-syndrome, refractory cytopenia with unilineage dysplasia, refractory anemia, refractory neutropenia, and refractory thrombocytopenia, refractory anemia with ring sideroblasts, refractory cytopenia with multilineage dysplasia (RCMD), refractory anemias with excess blasts (REAB) I and II, refractory anemia with excess blasts in transformation (RAEB-T), chronic myelomonocytic leukemia (CMML), myelodysplasia unclassifiable, refractory cytopenia of childhood (dysplasia in childhood).

A selected tricyclic heterobifunctional compound of the present invention, or pharmaceutical salt thereof, optionally in a pharmaceutical composition as described herein can also be administered in an effective amount to a host to degrade Family with Sequence Similarity 83, Member H (FAM83H). FAM83H is believed to be involved in the progression of human cancers in conjunction with tumor-associated molecules, such as MYC and β-catenin, and overexpression has been associated with lung, breast, colon, liver, ovary, pancreas, prostate, esophageal, glioma, hepatocellular carcinoma, thyroid, renal cell carcinoma, osteosarcoma, and stomach cancers (see, e.g., Kim et al., FAM83H is involved in stabilization of β-catenin and progression of osteosarcomas. Journal of Experimental & Clinical Cancer Research volume 38, Article number: 267 (2019)). Accordingly, in some embodiments, a compound of the present invention, or pharmaceutical salt thereof, optionally in a pharmaceutical composition as described herein is used to degrade FAM83H for the treatment of a cancer, including but not limited to, lung, breast, colon, liver, ovary, pancreas, prostate, esophageal, glioma, thyroid, liver cancer, including but not limited to hepatocellular carcinoma, renal cell carcinoma, osteosarcoma, and stomach cancers.

Alternatively, a selected tricyclic heterobifunctional compound of the present invention, or pharmaceutical salt thereof, optionally in a pharmaceutical composition as described herein can be administered in an effective amount to a host to degrade Zinc-finger and BTB domain containing protein 16 (ZBTB16). Overexpression and translocation of ZBTB16 has been associated with the development of various hematological cancers, including acute promyelocytic leukemia (see, e.g., Zhang et al., Genomic sequence, structural organization, molecular evolution, and aberrant rearrangement of promyelocytic leukemia zinc finger gene. Proc. Nat. Acad. Sci. 96: 11422-11427, 1999). Accordingly, in some embodiments, a compound of the present invention, or pharmaceutical salt thereof, optionally in a pharmaceutical composition as described herein is used to degrade ZBTB16 for the treatment of a cancer, including but not limited to a hematological cancer including but not limited to a leukemia or lymphoma, including but not limited to acute promyelocytic leukemia, acute lymphoblastic leukemia, Adult T-cell lymphoma/ATL, and Burkitt's lymphoma.

In an alternative embodiment, a selected tricyclic heterobifunctional compound of the present invention, or pharmaceutical salt thereof, optionally in a pharmaceutical composition as described herein can be administered in an effective amount to a host to degrade AT-Rich Interaction Domain-Containing Protein 2 (ARID2). ARID2 is a subunit of the PBAF chromatin-remodeling complex, which facilitates ligand-dependent transcriptional activation by nuclear receptors (see, e.g., Yan et al., PBAF chromatin-remodeling complex requires a novel specificity subunit, BAF200, to regulate expression of selective interferon-responsive genes. Genes Dev. 19: 1662-1667, 2005).

In another aspect, a selected tricyclic heterobifunctional compound of the present invention, or pharmaceutical salt thereof, optionally in a pharmaceutical composition as described herein can be administered in an effective amount to a host to degrade Polybromo associated BAF (PBAF). Mutations in PBAF have been associated with the development of synovial sarcomas and multiple myeloma (see, e.g., Alfert et al., The BAF complex in development and disease. Epigenetics & Chromatin volume 12, Article number: 19 (2019)). Accordingly, in some embodiments, a compound of the present invention, or pharmaceutical salt thereof, optionally in a pharmaceutical composition as described herein is used to degrade PBAF for the treatment of a cancer, including but not limited to synovial sarcoma and multiple myeloma.

In other embodiments, the selected tricyclic heterobifunctional compound of the present invention when administered after binding to and forming a neomorphic surface with cereblon, is capable of binding a number of neosubstrates resulting in a form of “poly-pharmacology.” For example, the tricyclic compound may bind and degrade IRAK4, IKZF1 and/or 3, and or Aiolos. In other examples, the tricyclic compound, when administered, is able to degrade two or more of the proteins named above or herein, for example, SALL4 and IKZF 1/3 or IKZF2/4.

The Target Protein is recruited with a Targeting Ligand, which is a ligand for the Target Protein. Typically the Targeting Ligand binds the Target Protein in a non-covalent fashion. In an alternative embodiment, the Target Protein is covalently bound to the Degron in a manner that can be irreversible or reversible.

In certain embodiments, the selected Target Protein is expressed from a gene that has undergone an amplification, translocation, deletion, or inversion event which causes or is caused by a medical disorder. In certain aspects, the selected Target Protein has been post-translationally modified by one, or a combination, of phosphorylation, acetylation, acylation including propionylation and crotylation, N-linked glycosylation, amidation, hydroxylation, methylation and poly-methylation, O-linked glycosylation, pyrogultamoylation, myristoylation, farnesylation, geranylgeranylation, ubiquitination, sumoylation, or sulfation which causes or is caused by a medical disorder.

As contemplated herein, the present invention includes a tricyclic cereblon binding heterobifunctional degrader with a Targeting Ligand that binds to a Target Protein of interest. The Target Protein is any amino acid sequence to which a Degrader can be bound which by degradation thereof, causes a beneficial therapeutic effect in vivo. In certain embodiments, the Target Protein is a non-endogenous peptide such as that from a pathogen or toxin. In another embodiment, the Target Protein can be an endogenous protein that mediates a disorder. The endogenous protein can be either the normal form of the protein or an aberrant form. For example, the Target Protein can be a mutant protein found in cancer cells, or a protein, for example, where a partial, or full, gain-of-function or loss-of-function is encoded by nucleotide polymorphisms. In some embodiments, the Degrader targets the aberrant form of the protein and not the normal form of the protein. In another embodiment, the Target Protein can mediate an inflammatory disorder or an immune disorder, including an auto-immune disorder. In certain embodiments, the Target Protein is a non-endogenous protein from a virus, as non-limiting examples, HIV, HBV, HCV, RSV, HPV, CMV, flavivirus, pestivirus, coronavirus, noroviridae, etc. In certain embodiments, the Target Protein is a non-endogenous protein from a bacteria, which may be for example, a gram positive bacteria, gram negative bacteria or other, and can be a drug-resistant form of bacteria. In certain embodiments, the Target Protein is a non-endogenous protein from a fungus. In certain embodiments, the Target Protein is a non-endogenous protein from a prion. In certain embodiments, the Target Protein is a protein derived from a eukaryotic pathogen, for example a protist, helminth, etc.

In one aspect, the Target Protein mediates chromatin structure and function. The Target Protein may mediate an epigenetic action such as DNA methylation or covalent modification of histones. An example is histone deacetylase (HDAC 1, 2, 3, 4, 5, 6, 7, 8, 9, 10 or 11). Alternatively, the Target Protein may be a bromodomain, which are readers of lysine acetylation (for example, BRD1, 2, 3, 4, 5, 6, 7, 8, 9 and T. FIG. 9 illustrates the proteins of the bromodomain family, which, for example, can act as Target Proteins according to the present invention.

Other nonlimiting examples of Target Proteins are a structural protein, receptor, enzyme, cell surface protein, a protein involved in apoptotic signaling, aromatase, helicase, mediator of a metabolic process (anabolism or catabolism), antioxidant, protease, kinase, oxidoreductase, transferase, hydrolase, lyase, isomerase, ligase, enzyme regulator, signal transducer, structural molecule, binding activity (protein, lipid carbohydrate), cell motility protein, membrane fusion protein, cell communication mediator, regulator of biological processes, behavioral protein, cell adhesion protein, protein involved in cell death, protein involved in transport (including protein transporter activity, nuclear transport, ion transporter, channel transporter, carrier activity, permease, secretase or secretion mediator, electron transporter, chaperone regulator, nucleic acid binding, transcription regulator, extracellular organization and biogenesis regulator, and translation regulator).

In certain embodiments, the Target Protein is a modulator of a signaling cascade related to a known disease state. In another embodiment, the Target Protein mediates a disorder by a mechanism different from modulating a signaling cascade. Any protein in a eukaryotic system or a microbial system, including a virus, bacteria or fungus, as otherwise described herein, are targets for proteasomal degradation using the present invention. The Target Protein may be a eukaryotic protein, and in some embodiments, a human protein.

In certain embodiments, the Target Protein is RXR, DHFR, Hsp90, a kinase, HDM2, MDM2, BET bromodomain-containing protein, HDAC, IDH1, Mcl-1, human lysine methyltransferase, a nuclear hormone receptor, aryl hydrocarbon receptor (AHR), RAS, RAF, FLT, SMARC, KSR, NF2L, CTNB, CBLB, BCL.

In certain embodiments, a bromodomain containing protein has histone acetyl transferase activity.

In certain embodiments, the bromodomain containing protein is BRD2, BRD3, BRD4, BRDT or ASH1L.

In certain embodiments, the bromodomain containing protein is a non-BET protein.

In certain embodiments, the non-BET protein is BRD7 or BRD9.

In certain embodiments, the FLT is not FLT 3. In certain embodiments, the RAS is not RASK. In certain embodiments, the RAF is not RAF1. In certain embodiments, the SMARC is not SMARC2. In certain embodiments, the KSR is not KSR1. In certain embodiments, the NF2L is not NF2L2. In certain embodiments, the CTNB is not CTNNB1. In certain embodiments, the BCL is not BCL6.

In certain embodiments, the Target Protein is selected from: EGFR, FLT3, RAF1, SMARCA2, KSR1, NF2L2, CTNNB1, CBLB, BCL6, and RASK.

In another embodiment, the Target Protein is not selected from: EGFR, FLT3, RAF1, SMARCA2, KSR1, NF2L2, CTNNB1, CBLB, BCL6, and RASK.

In certain embodiments, the Targeting Ligand is an EGFR ligand, a FLT3 ligand, a RAF1 ligand, a SMARCA2 ligand, a KSR1 ligand, a NF2L2 ligand, a CTNNB1 ligand, a CBLB ligand, a BCL6 ligand, or a RASK ligand.

In certain embodiments, the Targeting Ligand is not a EGFR ligand, a FLT3 ligand, a RAF1 ligand, a SMARCA2 ligand, a KSR1 ligand, a NF2L2 ligand, a CTNNB1 ligand, a CBLB ligand, a BCL6 ligand, or a RASK ligand.

In certain embodiments the Targeting Ligand is not a STAT protein ligand.

In certain embodiments the Targeting Ligand is not an IRAK4 ligand.

The present invention may be used to treat a wide range of disease states and/or conditions, including any disease state and/or condition in which a protein is dysregulated and where a patient would benefit from the degradation of proteins.

For example, a Target Protein can be selected that is a known target for a human therapeutic, and the therapeutic can be used as the Targeting Ligand when incorporated into the Degrader according to the present invention. These include proteins which may be used to restore function in a polygenic disease, including for example B7.1 and B7, TINFRlm, TNFR2, NADPH oxidase, Bcl2/Bax and other partners in the apoptosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, e.g., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuraminidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VCAM, VLA-4 integrin, selectins, CD40/CD40L, neurokinins and receptors, inosine monophosphate dehydrogenase, p38 MAP Kinase, Ras/Raf/MER/ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-2/neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further Target Proteins include Acetyl-CoA carboxylase, adenylosuccinate synthetase, protoporphyrinogen oxidase, and enolpyruvylshikimate-phosphate synthase.

In certain embodiments, the Target Protein is derived from a kinase to which the Targeting Ligand is capable of binding or binds including, but not limited to, a tyrosine kinase (e.g., AATK, ABL, ABL2, ALK, AXL, BLK, BMX, BTK, CSF1R, CSK, DDR1, DDR2, EGFR, EPHA1, EPHA2, EPHA3, EPHA4, EPHA5, EPHA6, EPHA7, EPHA8, EPHA10, EPHB1, EPHB2, EPHB3, EPHB4, EPHB6, ERBB2, ERBB3, ERBB4, FER, FES, FGFR1, FGFR2, FGFR3, FGFR4, FGR, FLT1, FLT3, FLT4, FRK, FYN, GSG2, HCK, IGF1R, ILK, INSR, INSRR, IRAK4, ITK, JAK1, JAK2, JAK3, KDR, KIT, KSR1, LCK, LMTK2, LMTK3, LTK, LYN, MATK, MERTK, MET, MLTK, MST1R, MUSK, NPR1, NTRK1, NTRK2, NTRK3, PDGFRA, PDGFRB, PLK4, PTK2, PTK2B, PTK6, PTK7, RET, ROR1, ROR2, ROS1, RYK, SGK493, SRC, SRMS, STYK1, SYK, TEC, TEK, TEX14, TIE1, TNK1, TNK2, TNNI3K, TXK, TYK2, TYRO3, YES1, or ZAP70).

In certain embodiments, the Target Protein is derived from a kinase to which the Targeting Ligand is capable of binding or binds including, but not limited to, a serine/threonine kinase (e.g., casein kinase 2, protein kinase A, protein kinase B, protein kinase C, Raf kinases, CaM kinases, AKT1, AKT2, AKT3, ALK1, ALK2, ALK3, ALK4, Aurora A, Aurora B, Aurora C, CHK1, CHK2, CLK1, CLK2, CLK3, DAPK1, DAPK2, DAPK3, DMPK, ERK1, ERK2, ERK5, GCK, GSK3, HIPK, KHS1, LKB1, LOK, MAPKAPK2, MAPKAPK, MNK1, MSSK1, MST1, MST2, MST4, NDR, NEK2, NEK3, NEK6, NEK7, NEK9, NEK11, PAK1, PAK2, PAK3, PAK4, PAK5, PAK6, PIM1, PIM2, PLK1, RIP2, RIP5, RSK1, RSK2, SGK2, SGK3, SIK1, STK33, TAO1, TAO2, TGF-beta, TLK2, TSSK1, TSSK2, ULK1, or ULK2).

In certain embodiments, the Target Protein is derived from a kinase to which the Targeting Ligand is capable of binding or binds including, but not limited to a cyclin dependent kinase for example CDK1, CDK2, CDK3, CDK4, CDK5, CDK6, CDK7, CDK8, CDK9, CDK10, CDK11, CDK12, or CDK13.

In certain embodiments, the Target Protein is derived from a kinase to which the Targeting Ligand is capable of binding or binds including, but not limited to a leucine-rich repeat kinase (e.g., LRRK2).

In certain embodiments, the Target Protein is derived from a kinase to which the Targeting Ligand is capable of binding or binds including, but not limited to a lipid kinase (e.g., PIK3CA, PIK3CB) or a sphingosine kinase (e.g. SiP).

In certain embodiments, the Target Protein is derived from a BET bromodomain-containing protein to which the Targeting Ligand is capable of binding or binds including, but not limited to, ASH1L, ATAD2, BAZ1A, BAZ1B, BAZ2A, BAZ2B, BRD1, BRD2, BRD3, BRD4, BRD5, BRD6, BRD7, BRD8, BRD9, BRD10, BRDT, BRPF1, BRPF3, BRWD3, CECR2, CREBBP, EP300, FALZ, GCN5L2, KIAA1240, LOC93349, MLL, PB1, PCAF, PHIP, PRKCBP1, SMARCA2, SMARCA4, SP100, SP110, SP140, TAF1, TAF1L, TIF1a, TRIM28, TRIM33, TRIM66, WDR9, ZMYND11, and MLL4. In certain embodiments, a BET bromodomain-containing protein is BRD4.

In certain embodiments, the Target Protein is derived from a nuclear protein to which the Targeting Ligand is capable of binding or binds including, but not limited to, BRD2, BRD3, BRD4, Antennapedia Homeodomain Protein, BRCA1, BRCA2, CCAAT-Enhanced-Binding Proteins, histones, Polycomb-group proteins, High Mobility Group Proteins, Telomere Binding Proteins, FANCA, FANCD2, FANCE, FANCF, hepatocyte nuclear factors, Mad2, NF-kappa B, Nuclear Receptor Coactivators, CREB-binding protein, p55, p107, p130, Rb proteins, p53, c-fos, c-jun, c-mdm2, c-myc, and c-rel.

In certain embodiments, the Target Protein is a member of the Retinoid X Receptor (RXR) family and the disorder treated is a neuropsychiatric or neurodegenerative disorder. In certain embodiments, the Target Protein is a member of the Retinoid X Receptor (RXR) family and the disorder treated is schizophrenia.

In certain embodiments, the Target Protein is dihydrofolate reductase (DHFR) and the disorder treated is cancer. In certain embodiments, the Target Protein is dihydrofolate reductase (DHFR) and the disorder treated is microbial.

In certain embodiments, the Target Protein is dihydrofolate reductase from Bacillus anthracis (BaDHFR) and the disorder treated is anthrax.

In certain embodiments, the Target Protein is Heat Shock Protein 90 (HSP90) and the disorder treated is cancer.

In certain embodiments, the Target Protein is a kinase or phosphatase and the disorder treated is cancer.

In certain embodiments, the Target Protein is HDM2 and or MDM2 and the disorder treated is cancer.

In certain embodiments, the Target Protein is a BET bromodomain containing protein and the disorder treated is cancer.

In certain embodiments, the Target Protein is a lysine methyltransferase and the disorder treated is cancer.

In certain embodiments, the Target Protein belongs to the RAF family and the disorder treated is cancer.

In certain embodiments, the Target Protein belongs to the FKBP family and the disorder treated is an autoimmune disorder. In certain embodiments, the Target Protein belongs to the FKBP family and the disorder treated is organ rejection. In certain embodiments, the Target Protein belongs to the FKBP family and the compound is given prophylactically to prevent organ failure.

In certain embodiments, the Target Protein is an androgen receptor and the disorder treated is cancer.

In certain embodiments, the Target Protein is an estrogen receptor and the disorder treated is cancer.

In certain embodiments, the Target Protein is a viral protein and the disorder treated is a viral infection. In certain embodiments, the Target Protein is a viral protein and the disorder treated is HIV, HPV, or HCV.

In certain embodiments, the Target Protein is an AP-1 or AP-2 transcription factor and the disorder treated is cancer.

In certain embodiments, the Target Protein is a HIV protease and the disorder treated is a HIV infection. In certain embodiments, the Target Protein is a HIV integrase and the disorder treated is a HIV infection. In certain embodiments, the Target Protein is a HCV protease and the disorder treated is a HCV infection. In certain embodiments, the treatment is prophylactic and the Target Protein is a viral protein.

In certain embodiments, the Target Protein is a member of the histone deacetylase (HDAC) family and the disorder is a neurodegenerative disorder. In certain embodiments, the Target Protein is a member of the histone deacetylase (HDAC) family and the disorder is Huntingon's, Parkinson's, Kennedy disease, amyotropic lateral sclerosis, Rubinstein-Taybi syndrome, or stroke.

In certain embodiments, Targeting Ligand forms a covalent bond with the Target Protein. Non-limiting examples of Target Proteins and Targeting Ligands utilizing a covalent bond include those described in “Covalent Inhibitors Design and Discovery” Eur J. Med. Chem. 2017 Sep. 29; 138:96-114. doi: 10.1016/j.ejmech.2017.06.019; “Lysine-Targeting Covalent Inhibitors.” Angew Chem Int Ed Engl. 2017 Aug. 29. doi: 10.1002/anie.201707630; “Inhibition of Mcl-1 Through Covalent Modification of a Noncatalytic Lysine Side Chain.” Nat Chem Biol. 2016 November; 12(11):931-936; “Proteome-wide Map of Targets of T790M-EGFR-Directed Covalent Inhibitors” Cell Chem. Biol. 2016 November: 24:1-13; “Global Profiling of Lysine Reactivity and Ligandability in the Human Proteome” Nat. Chem. 2017 Jul. 31, doi:10.1038/nchem.2826; “The Resurgence of Covalent Drugs” Nat. Rev. Drug Disc. 2011 10, 307-217; U.S. Pat. Nos. 8,008,309; and 9,790,226.

In an alternative embodiment, the Target Protein is selected from DOTL1, CBP, WDR5, BRAF, KRAS, MCL1, PTPN2, HER2, and SHOC2. In another alternative embodiment, the Target Protein is selected from UCHL1, USP6, USP14, and USP30. In another alternative embodiment, the Target Protein is selected from USP1, USP2, USP4, USP6, USP7, USP8, USP9x, USP10, USP 11, USP13, USP14, USP17, and USP28.

In an alternative embodiment, the Target Protein is selected from DOTL1, CBP, WDR5, BRAF, KRAS, MCL1, PTPN2, PTPN1, HER2, and SHOC2.

In certain embodiments the Target Protein is selected from Retinoid X Receptor (RXR), Dihydrofolate reductase (DHFR), Bacillus anthracis Dihydrofolate reductase (BaDHFR), Heat Shock Protein 90 (HSP90), Tyrosine Kinase, Aurora Kinase, ATM, ATR, BPTF, ALK, ABL, JAK2, MET, mTORC1, mTORC2, Mast/stem cell growth factor receptor (SCFR), IGF1R, HDM2, MDM2, HDAC, RAF Receptor, Androgen Receptor, Estrogen Receptor, Thyroid Hormone Receptor, HIV Protease, HIV Integrase, AP1, AP2, MCL-1, DNA-PK, elF4E, IDH1, RAS, RASK, MERTK, MER, EGFR, FLT3, SMARCA2, Cyclin Dependent Kinase 9 (CDK9), Cyclin Dependent Kinase 12, Cyclin Dependent Kinase 13, Glucocorticoid Receptor, RasG12C, Her3, Bcl-2, Bcl-XL, PPAR-gamma, BCR-ABL, BRAF, LRRK2, PDGFRα, RET, Fatty Acid Binding Protein, 5-Lipoxygenase Activating Protein (FLAP), Kringle Domain V 4BVV, Lactoylglutathione Lyase, mPGES-1, Factor Xa, Kallikrein 7, Cathepsin K, Cathepsin L, Cathepsin S, MTH1, MDM4, PARP1, PARP2, PARP3, PARP14, PARP15, PDZ domain, Phospholipase A2 domain, Protein S100-A7 2WOS, NRASQ61K, NRASQ61R, TEAD1, TEAD2, TEAD3, TEAD4, Saposin-B, Sec7, pp60 Src, Tank1, Ubc9 SUMO E2 ligase SF6D, Src, Src-AS1, Src-AS2, JAK3, MEK1, KIT, KSR1, CTNNB1, BCL6, PAK1, PAK4, TNIK, MEN1, ERK1, IDO1, CBP, ASH1L, ATAD2, YAP, BAZ2A, BAZ2B, BDRT, BDR9, SMARCA4, PB1, TRIM24 (TIF1a), BRPF1, CECR2, CREBBP, PCAF, PHIP, TAF1, Histone Deacetylase 2, Histone Deacetylase 4, Histone Deacetylase 6, Histone Deacetylase 7, Histone Deacetylase 8, Histone Acetyltransferase (KAT2B), WWTR1, A2aR, alpha-subunit of FTase and/or GGTase, ARG1, B-TrCP, CBX7, Cdc7/ASK, Cdc7-Dbf4, Histone Acetyltransferase (KAT2A), Histone Acetyltransferase Type B Catalytic Unit (HAT1), Cyclic AMP-dependent Transcription Factor (ATF2), Histone Acetyltransferase (KAT5), Lysine-specific histone demethylase 1A (KDM1A), DOT1L, EHMT1, ceacam-1, CENP-E, clAP1/2, DKC1, DMT3A, DNA Replication/Repair protein, DNA2, DNMT3B, E2F1, EFHD2/SWIPROSIN, Eg5, EMIl, ERCC1/XPF, EWS-FLI, FoxAl, GATA3, FOXP1, GCN2, GNAQ, GNA11, SETD2, SETD7, SETD8, SETDB1, SMYD2, SMYD3, SUV4-20H1, ErbB2 receptor, ErbB4 receptor, VEGFR1 receptor, VEGFR2 receptor, VEGFR3 receptor, PDGFRO receptor, receptor, Lyn receptor, Hck receptor, c-Met receptor, TrkB receptor, Axl receptor, Tie 2 receptor, Ros1 receptor, HGFR receptor, MST1R receptor, Lck receptor, Yes receptor, HER2, PNET receptor, RCC receptor, RAML receptor, SEGA receptor, PDGFR receptors, ErbB2 receptor, HK2, HSP70, IAPs, IQGAP1, LSF, MCT1, MCT4, MEF2B, MMP3, MMP14, MUC1, MyB, Myd88, FGFR1 receptor, FGFR2 receptor, FGFR3 receptor, FGFR4 receptor, PDGRF receptor, DDR1 receptor, PDGRα receptor, PDGRβ receptor, CDK4 receptor, CDK6 receptor, Fms receptor, T315I VEGFR receptor, FGFR receptor, Flt 3 receptor, Eph2A receptor, JAK1 receptor, FKBP12 receptor, mTOR receptor, CDK 8 receptor, CSF-1R receptor, MEK2 receptor, Brk receptor, PI3Ka receptor, GCN5 receptor, G9a (EHMT2), EZH2, EED, PRMT3, PRMT4, PRMT5, PRMT6, NR2F6, NSD1, P70S6K, PIN1, SERCA, SF3B1, Sirtuin 2, Skp2, SMAD3, SPOP, Tall, KDM1, KDM4, KDM5, KDM6, L3MBTL3, Menin, HDAC6, HDAC7, PTP1B, SHP2, TBK1, Trib2, TRIF, TS, XPO1, RASN, ARID1BScavenger mRNA-decapping enzyme DcpS, ALK, BTK, NTRK1, NTRK2, NTRK3, IDO, ERK2, ABL1, ABL2, ATK1, ATK2, BMX, CSK, EPHA3, EPHA4, EPHA7, EPHB4, FES, FYN, GSG2, INSR, HBV, CBL-B, ERK, WDR5, NSP3, IRAK4, NRAS, ADAR, ASCL1, PAX8, TP63, SARM1, Ataxin-2, KSR2, CXCR4, HDAC10, NSD2, WHSC1, RIT1, WRN, BAP1, EPAS1, HIF2a, GRB2, KMT2D, MLL2, MLL4, MLLT1, ENL, NSD3, PPM1D, WIP1, SOS1, TBXT, Brachyury, USP7, BKV, JCV, CK1α, GSPT1, ERF3, IFZV, TAU, CYP17A1, SALL4, FAM38, CYP20A1, HTT, NRF2, NFE2L2, P300, PIK3CA, SARM1, SNCA, MAPT, TCPTP, STAT3, MyD88, PTP4A3, SF3B1, ARID1B, and ARID2.

In certain embodiments, the Target Protein as referred to herein is named by the gene that expresses it. The person skilled in the art will recognize that when a gene is referred to as a Target Protein, the protein encoded by the gene is the Target Protein. For example, ligands for the protein SMCA2 which is encoded by SMRCA2 are referred to as SMRCA2 Targeting Ligands.

### VI. Targeting Ligands

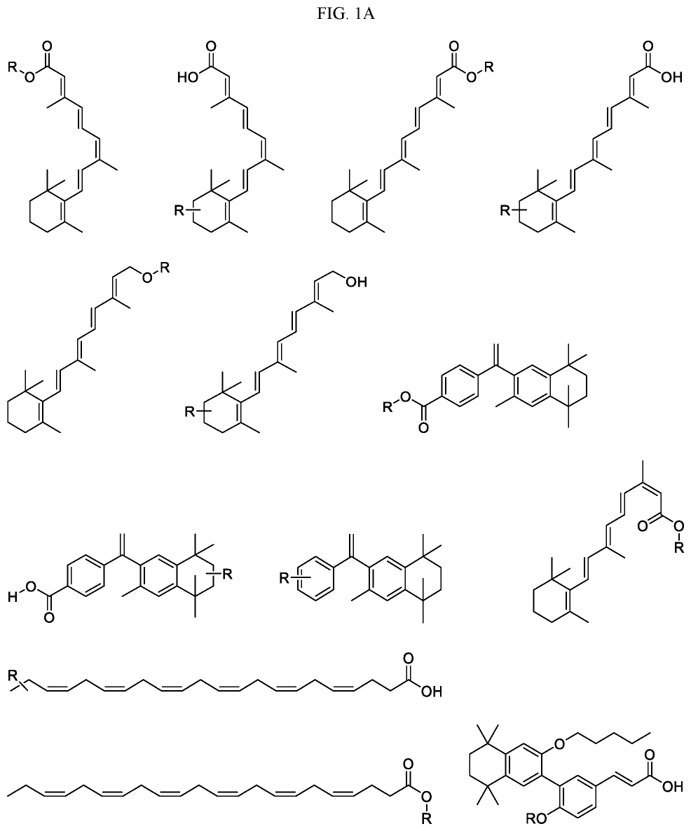

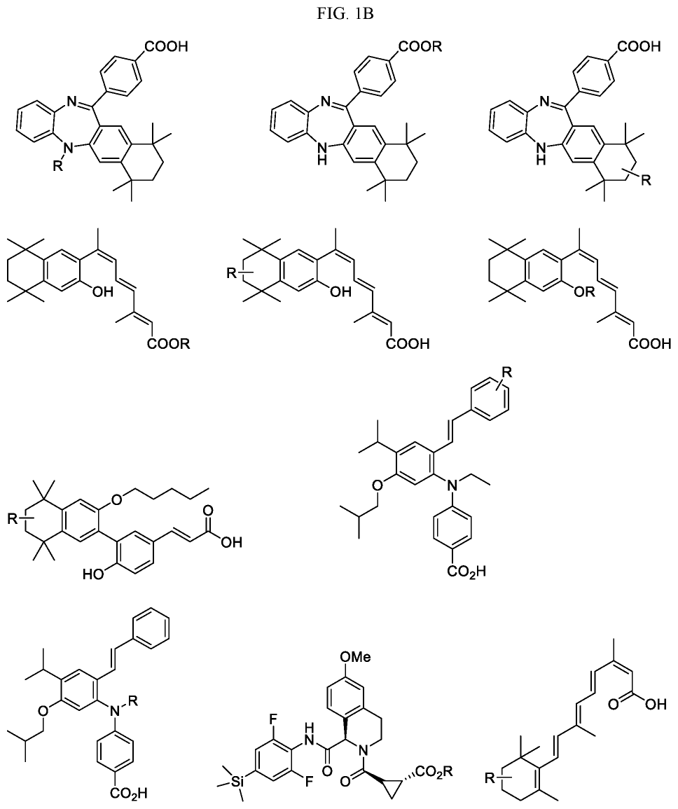

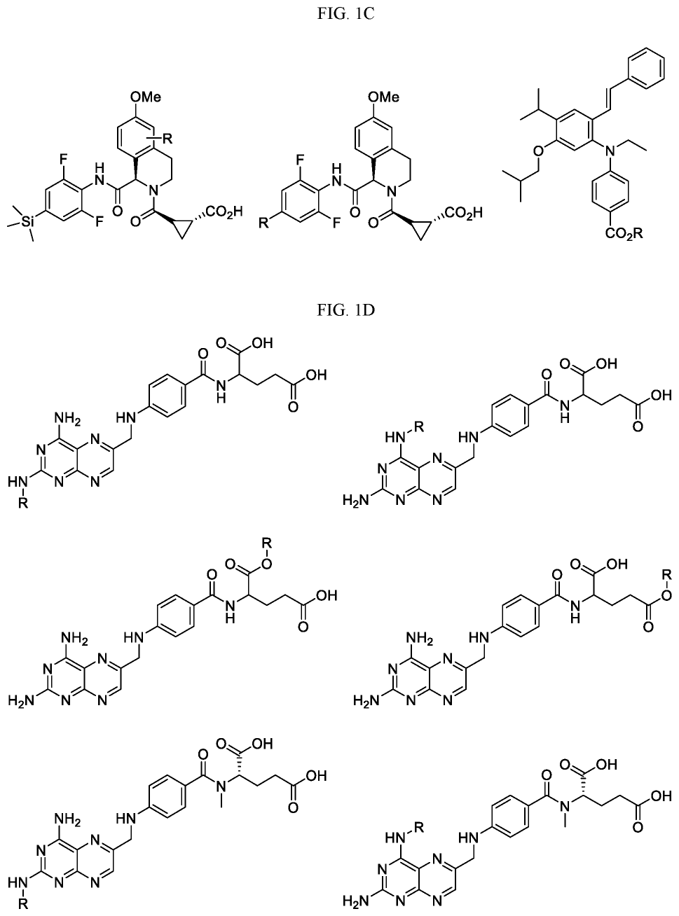

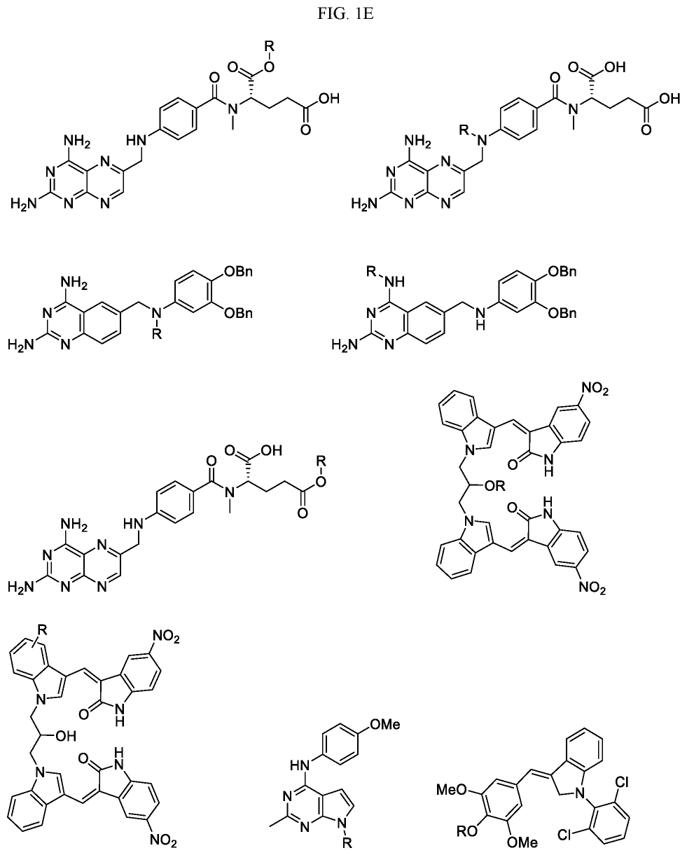

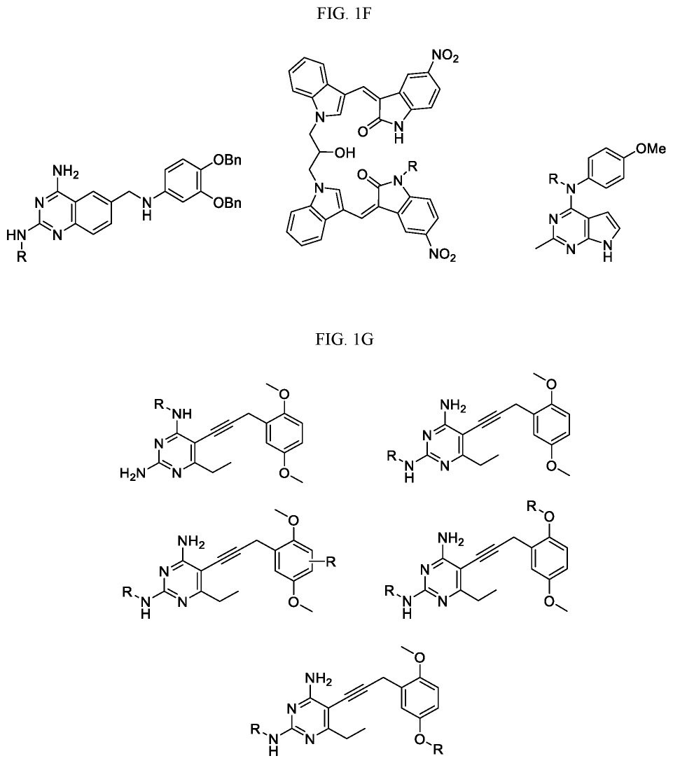

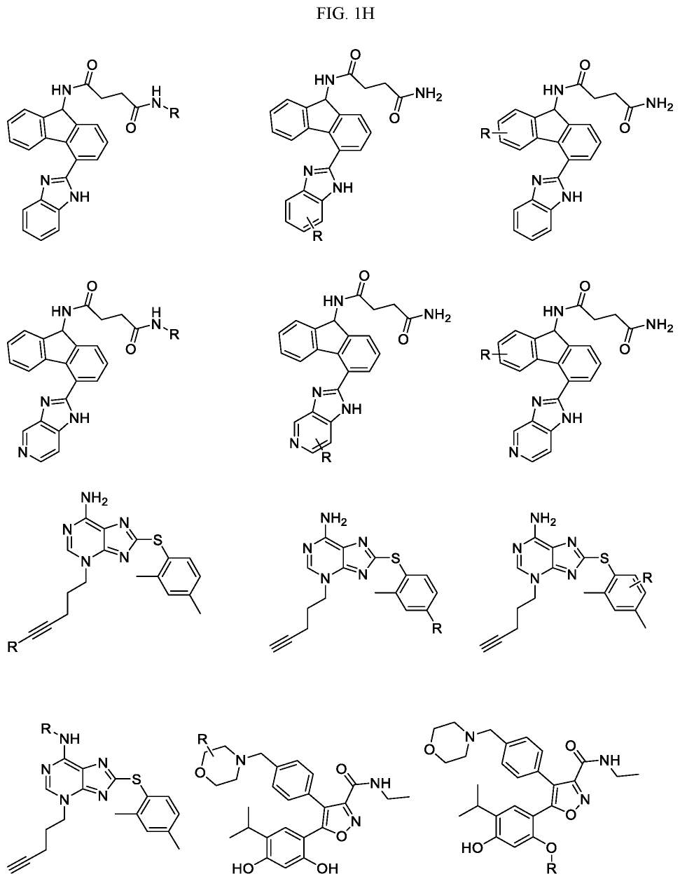

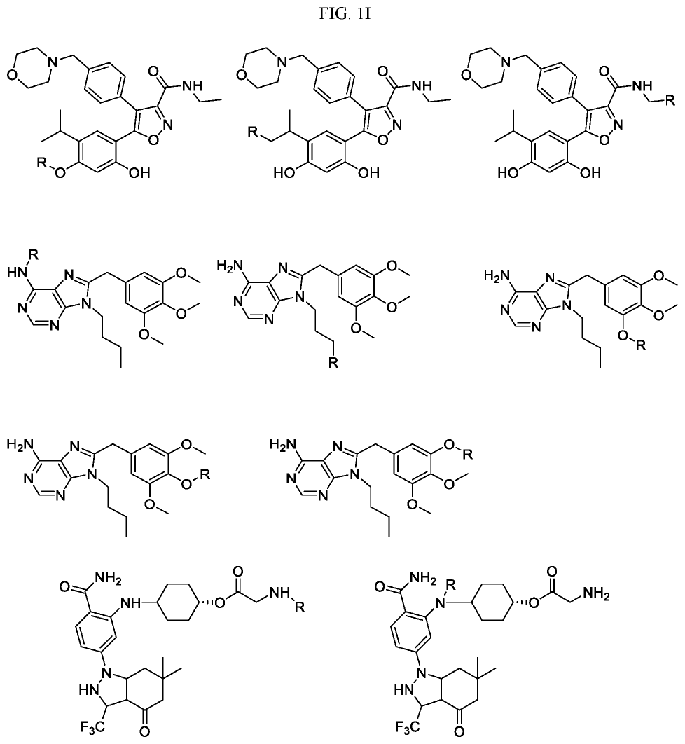

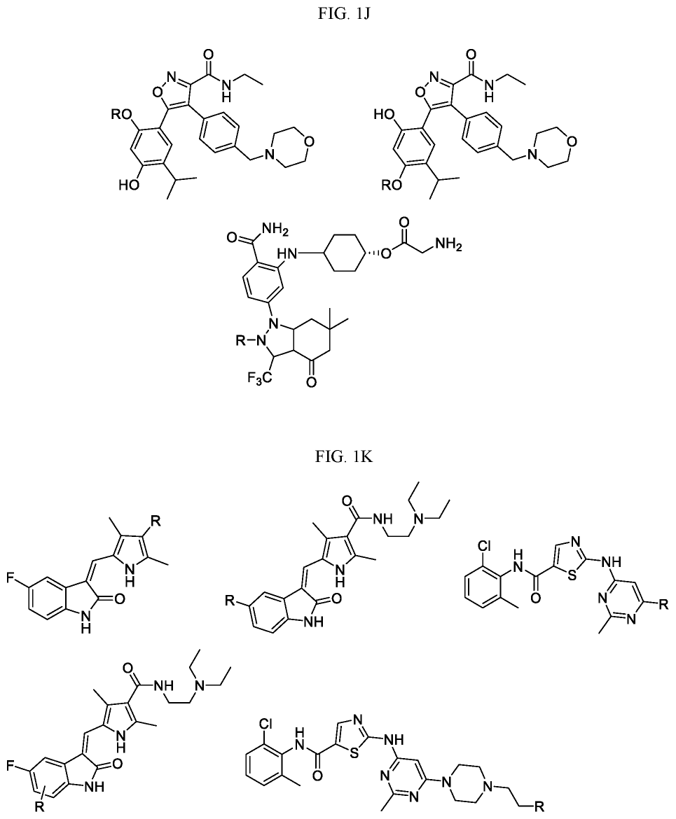

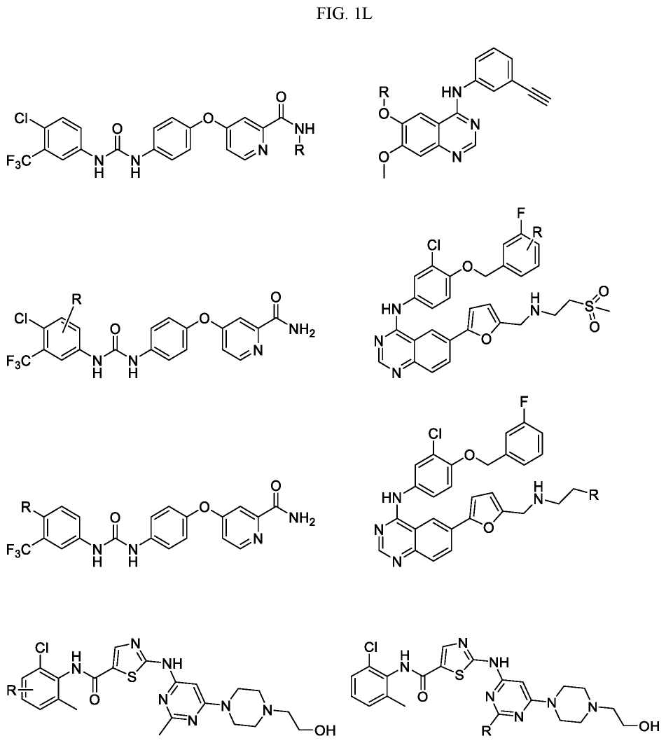

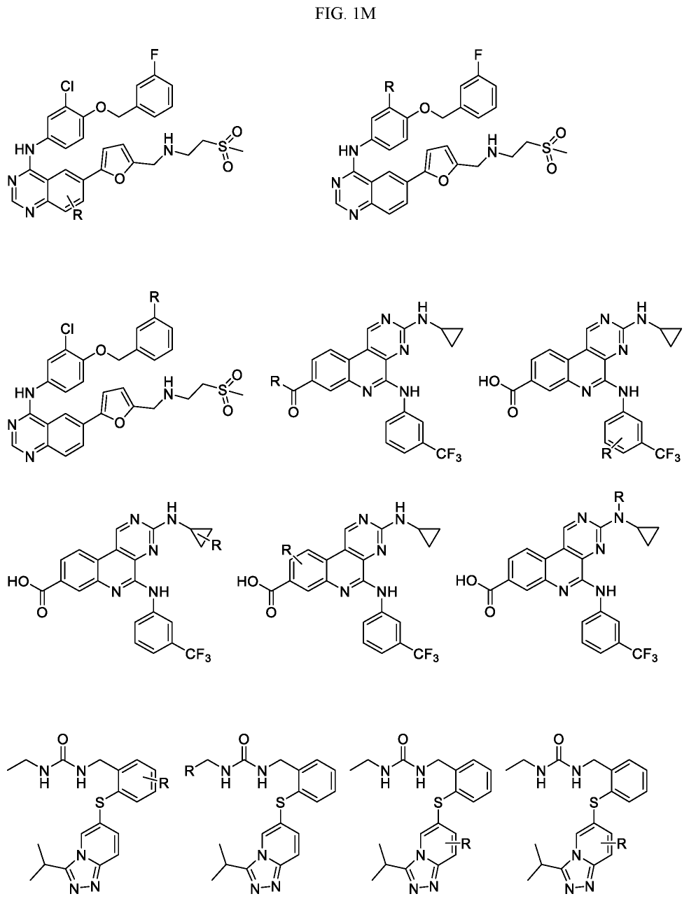

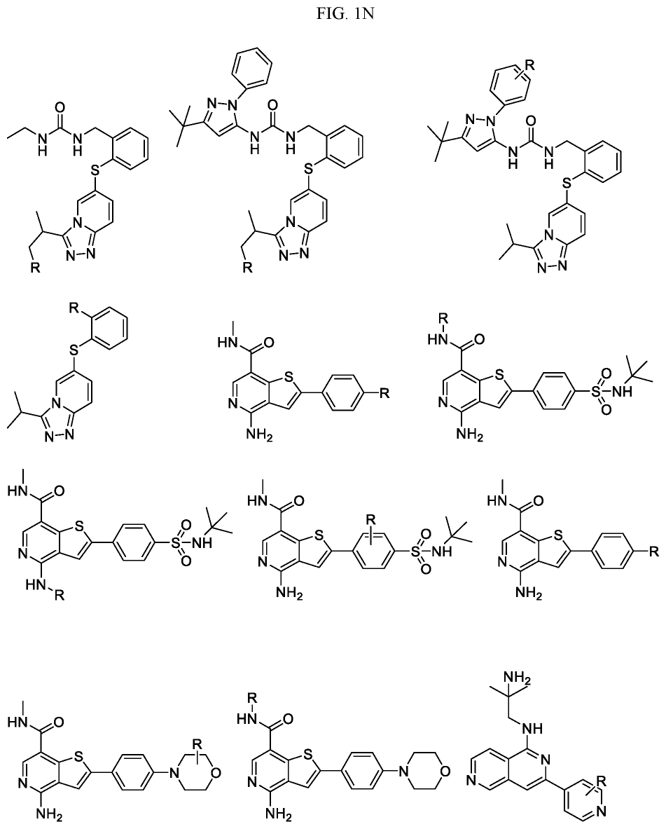

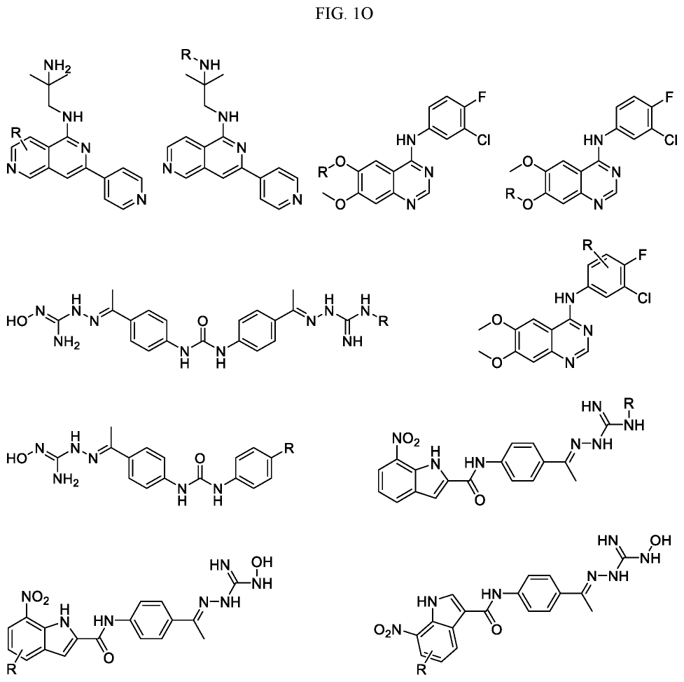

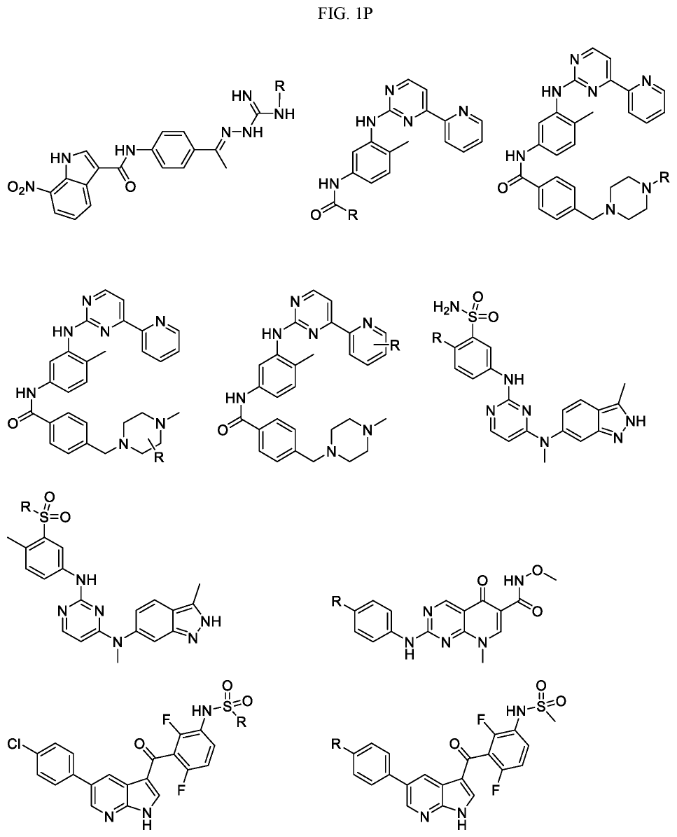

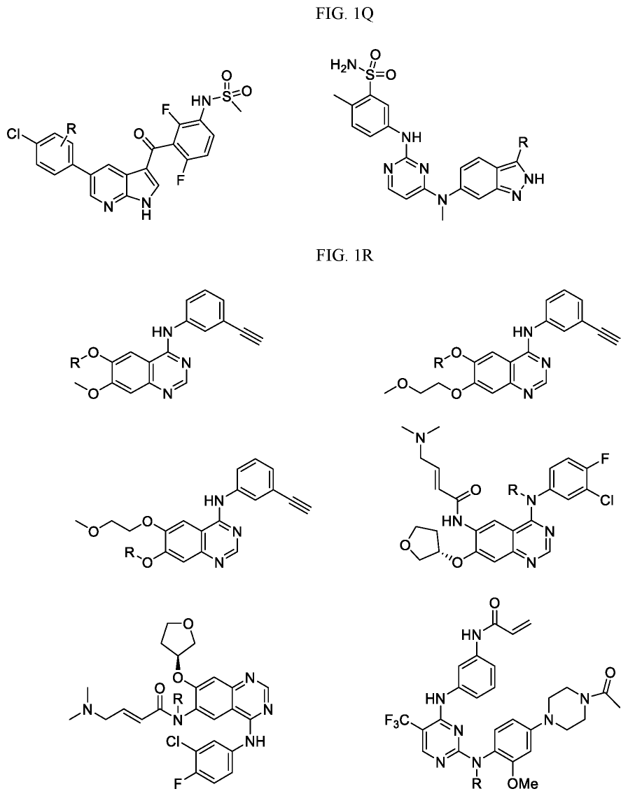

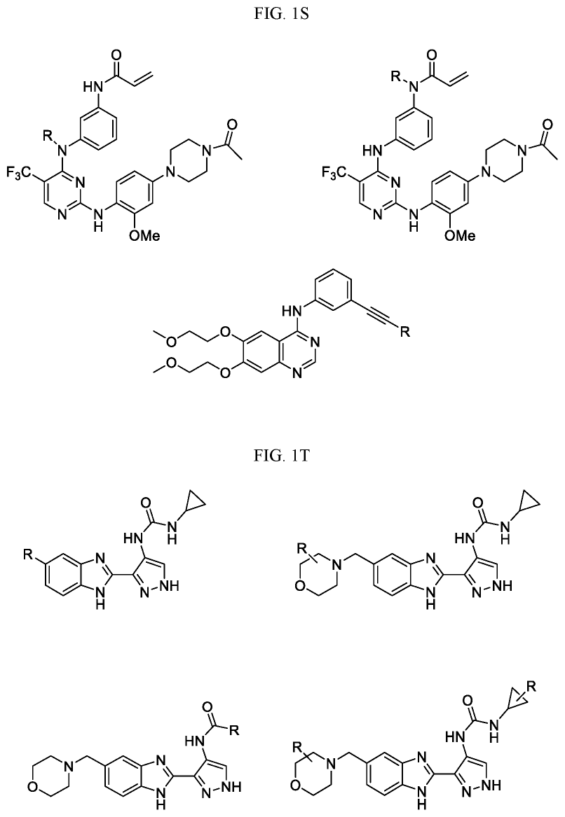

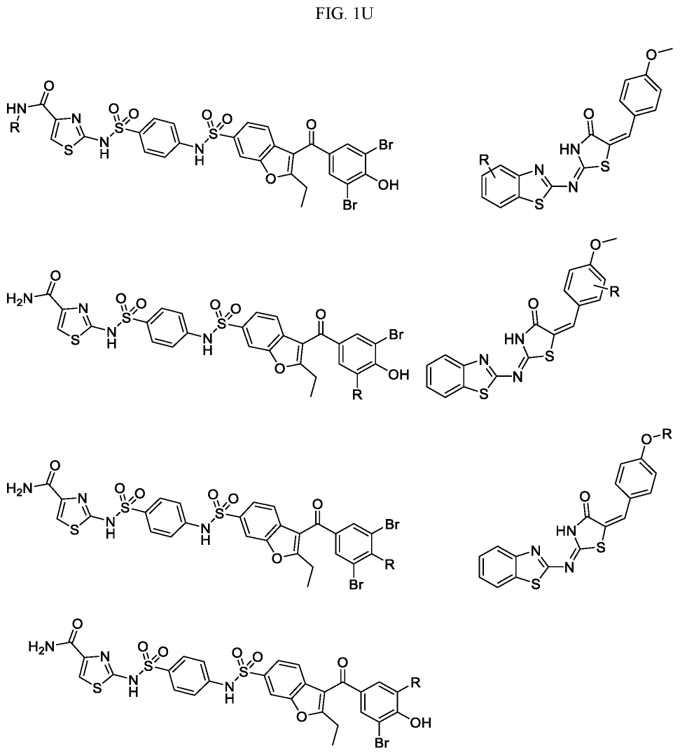

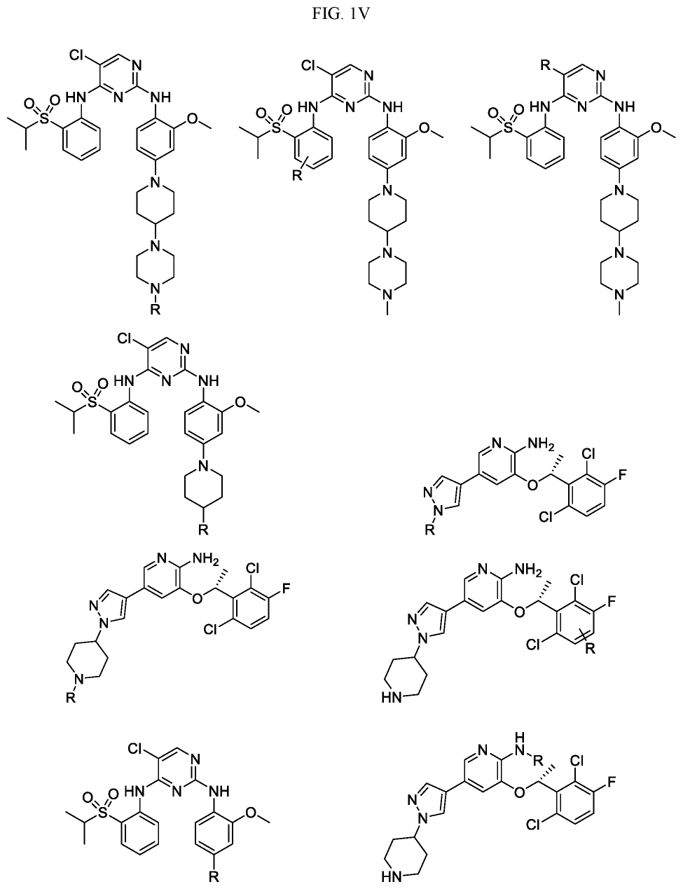

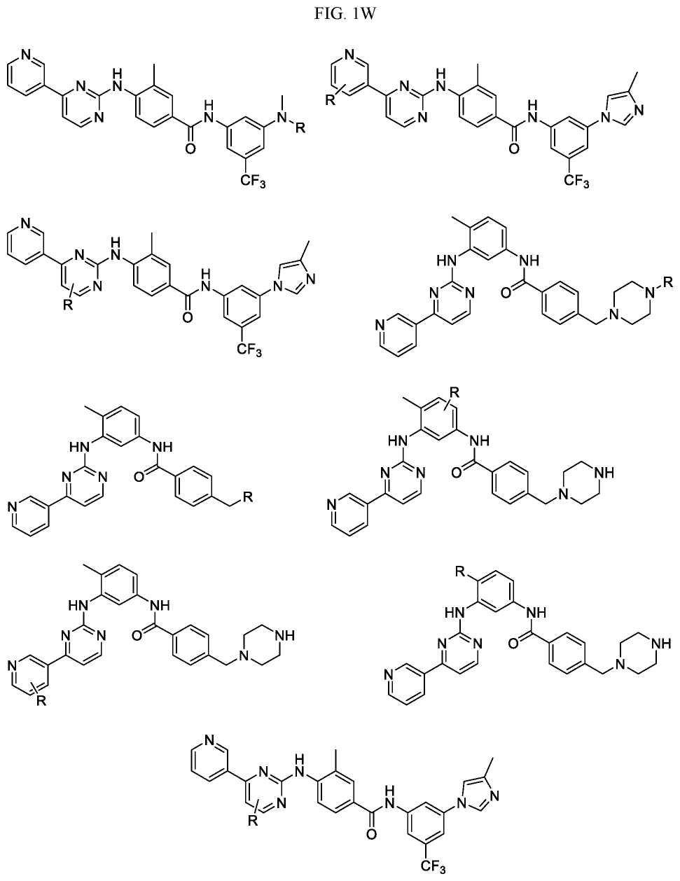

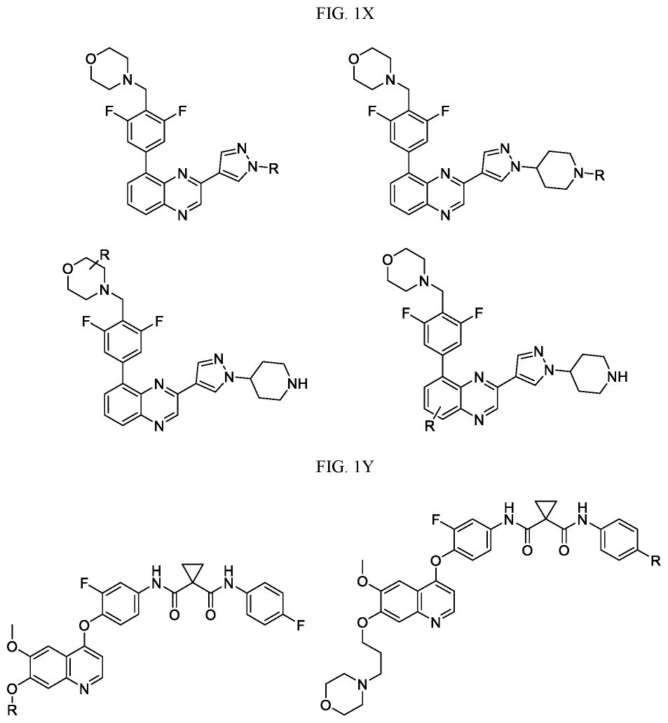

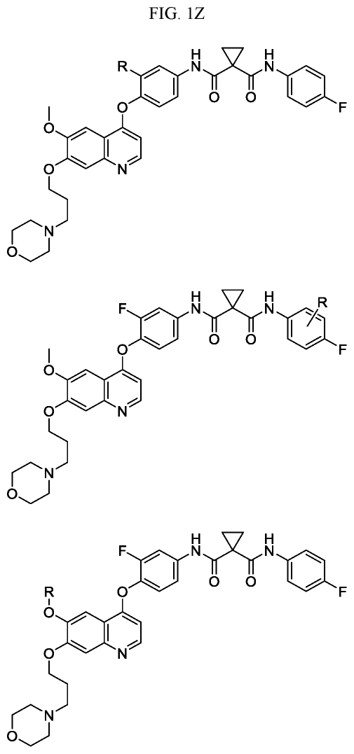

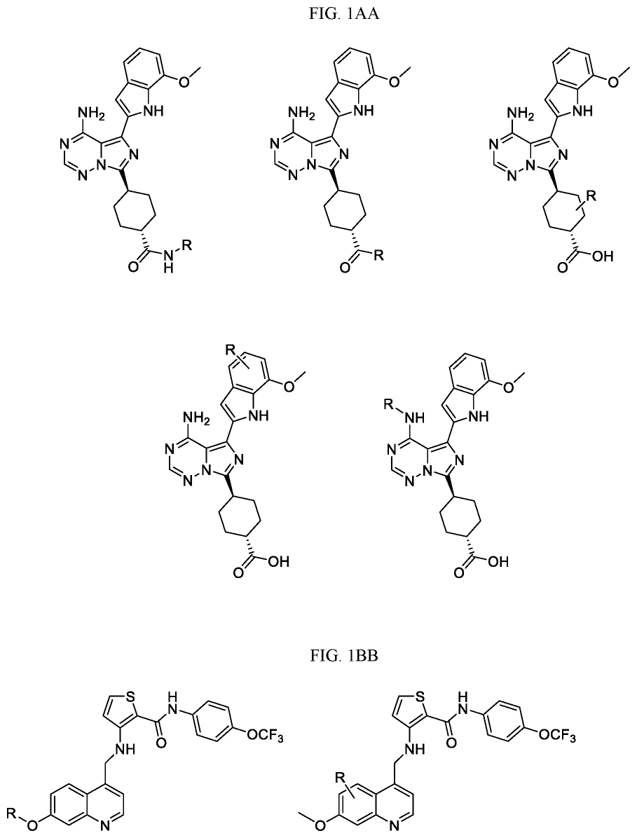

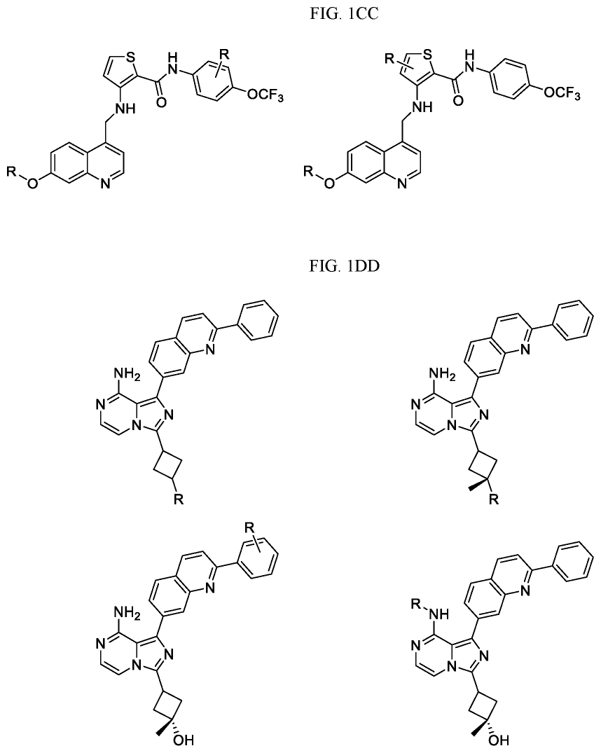

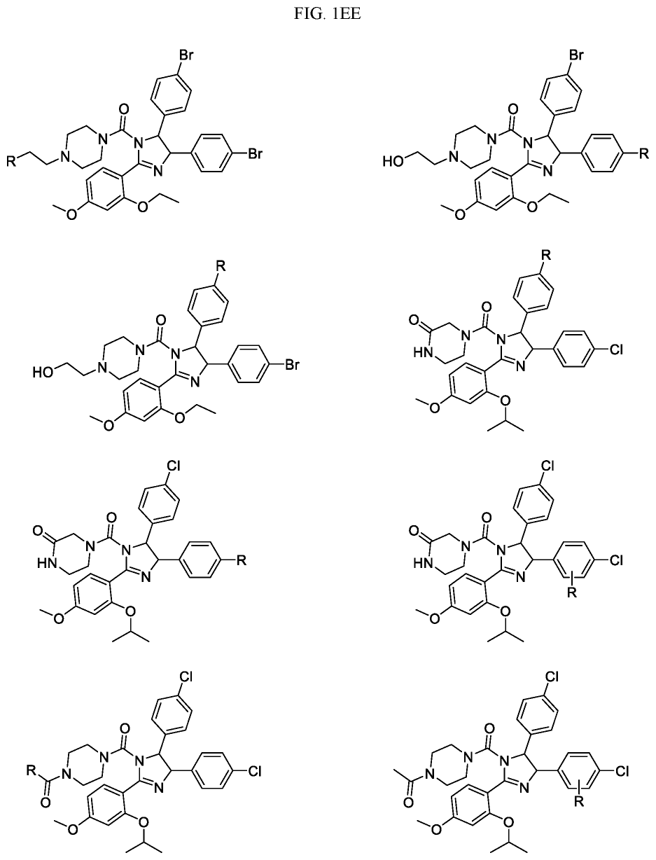

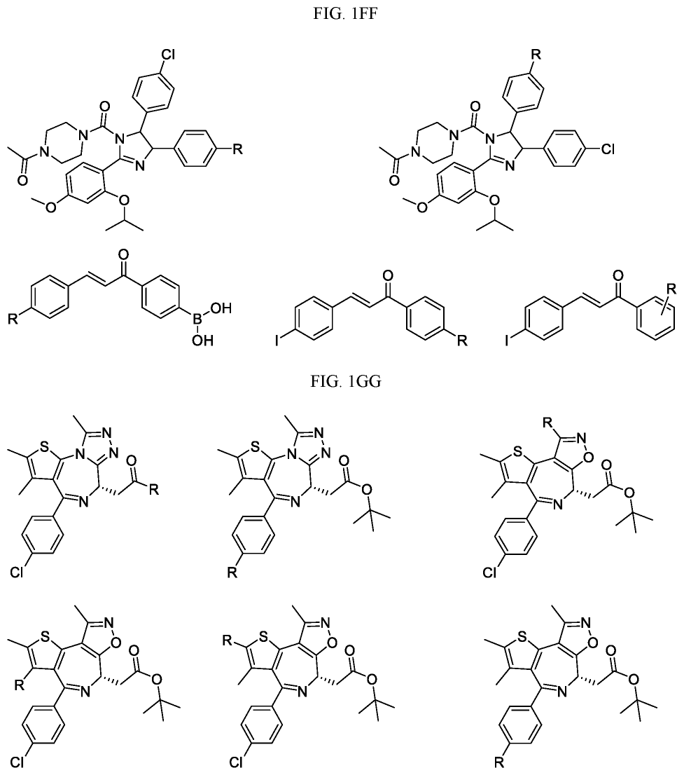

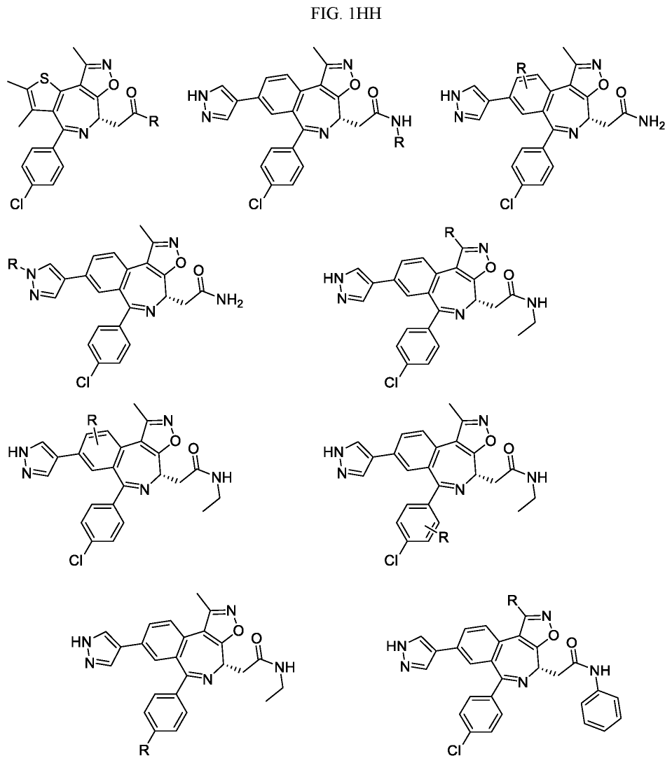

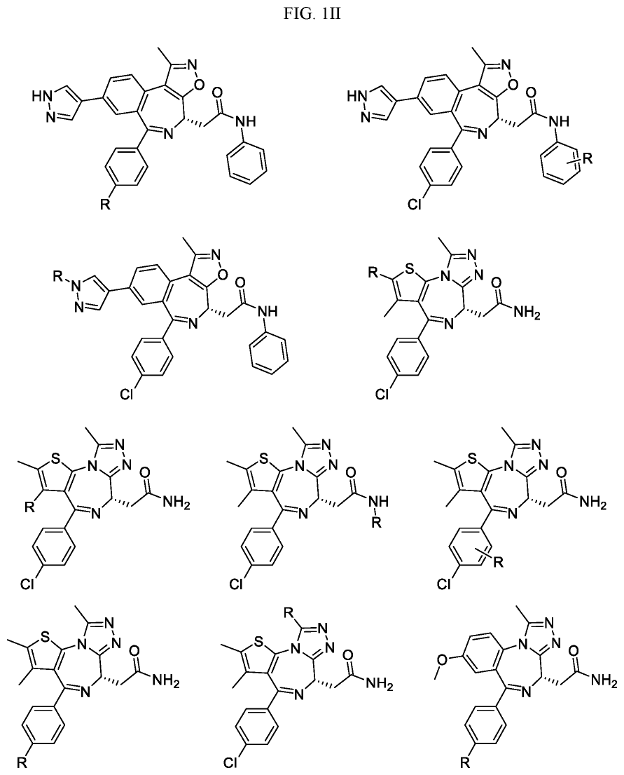

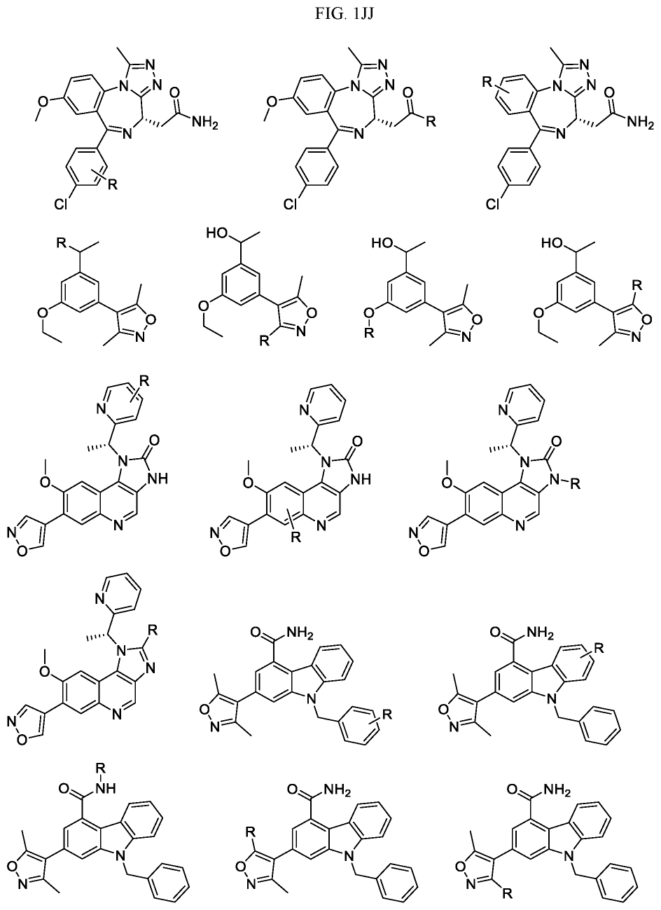

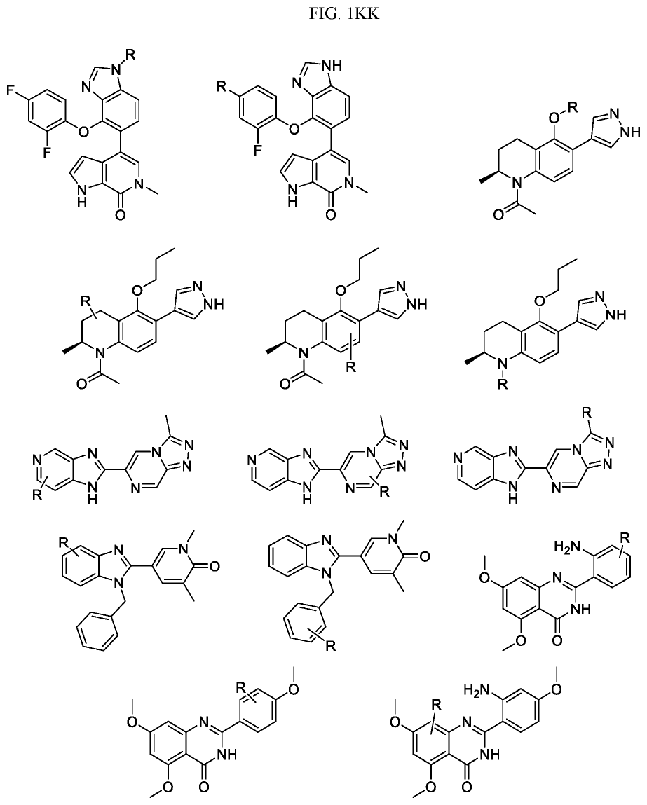

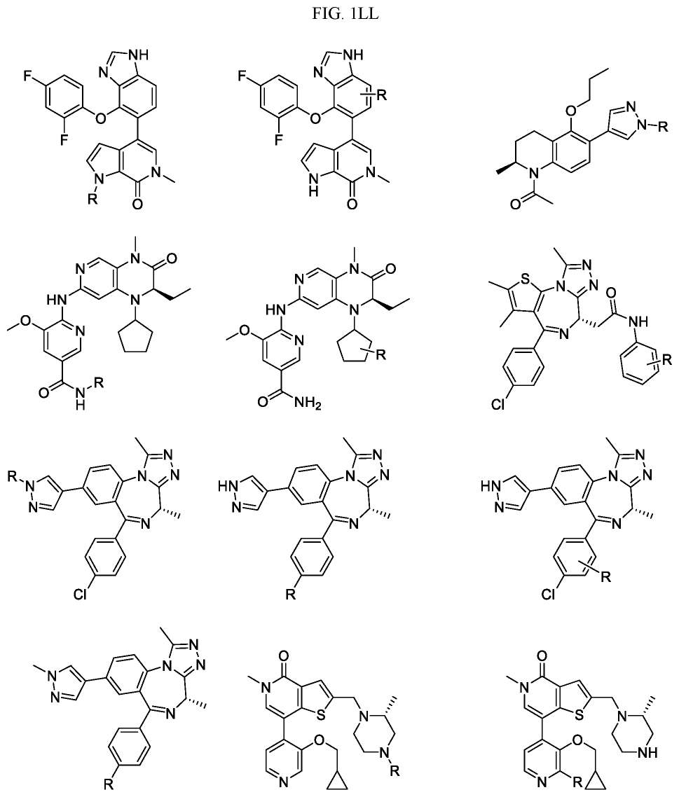

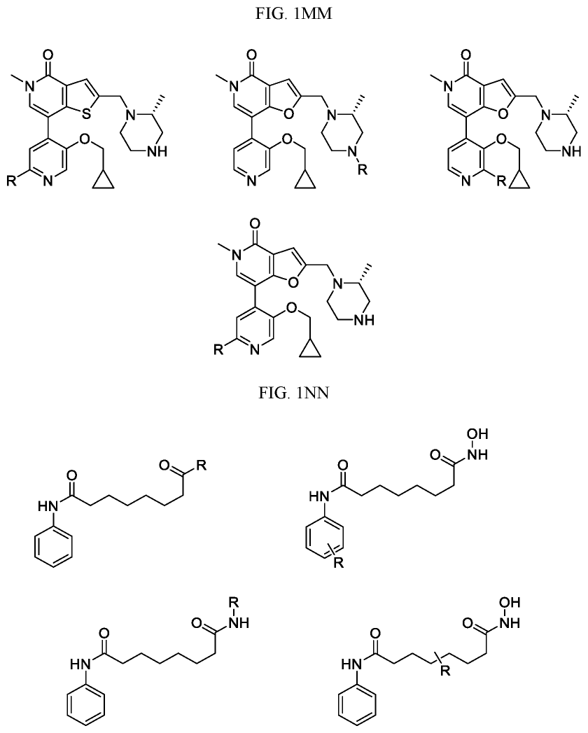

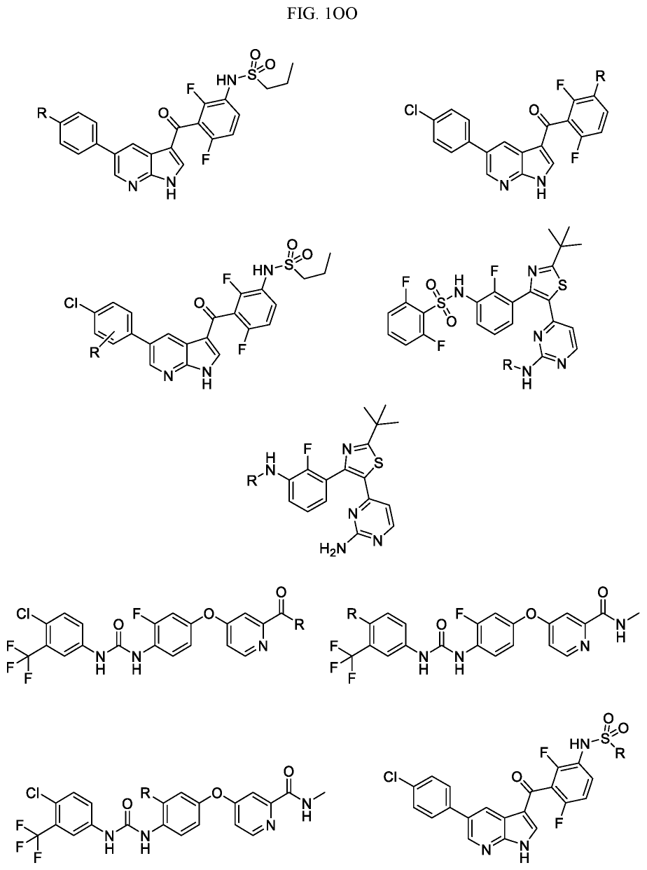

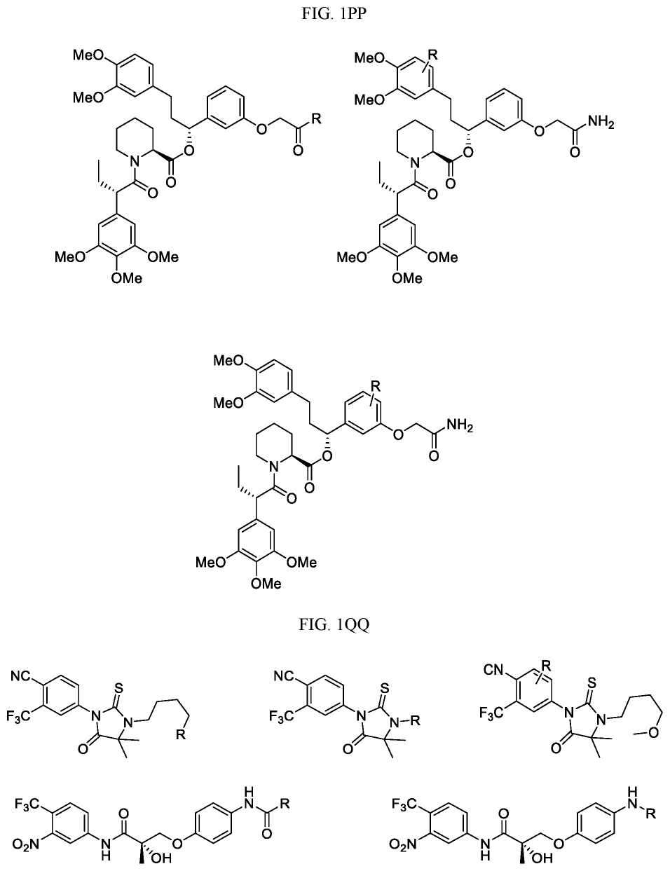

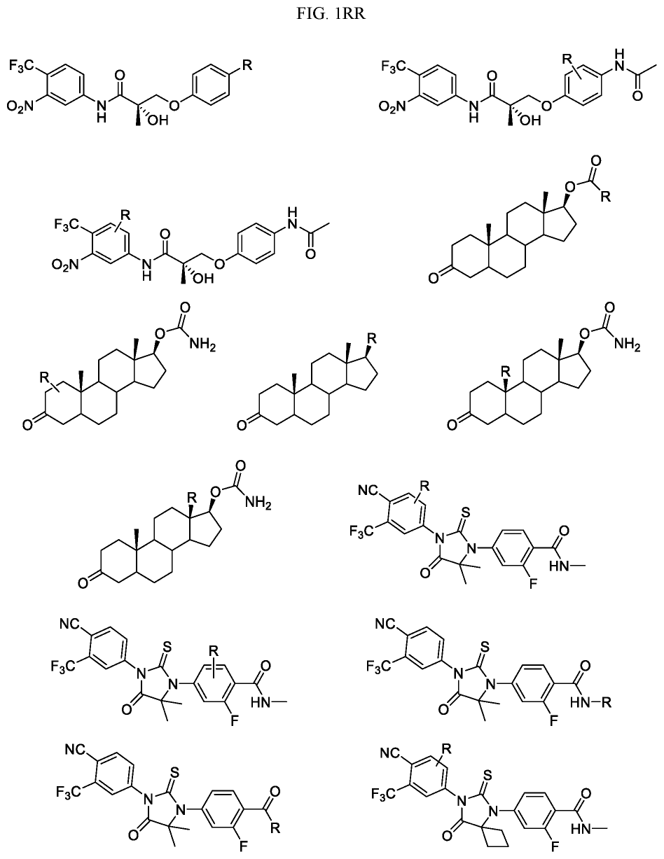

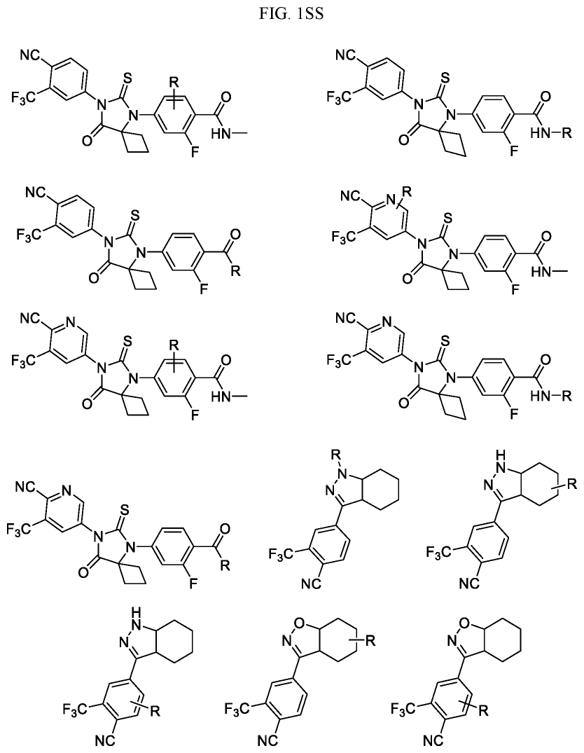

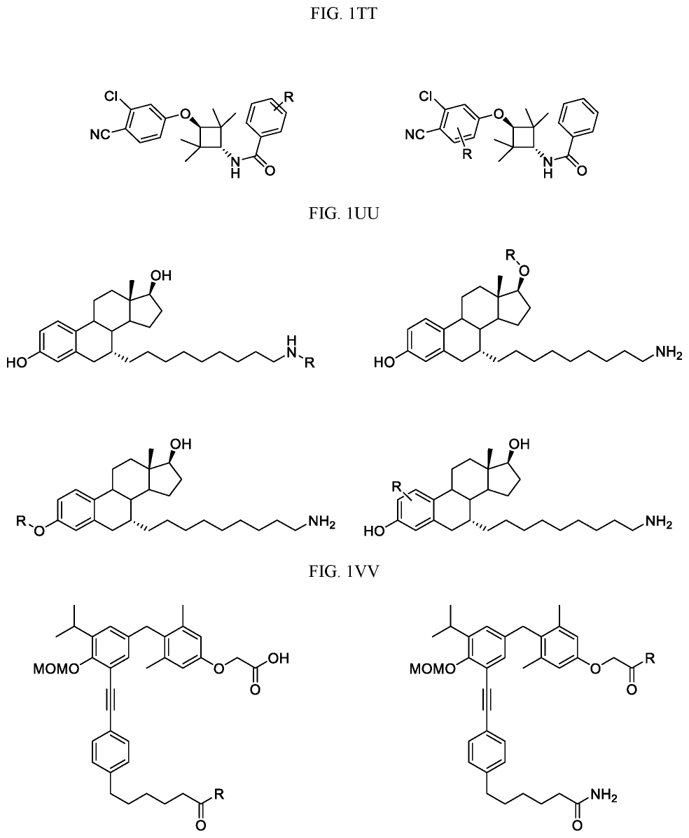

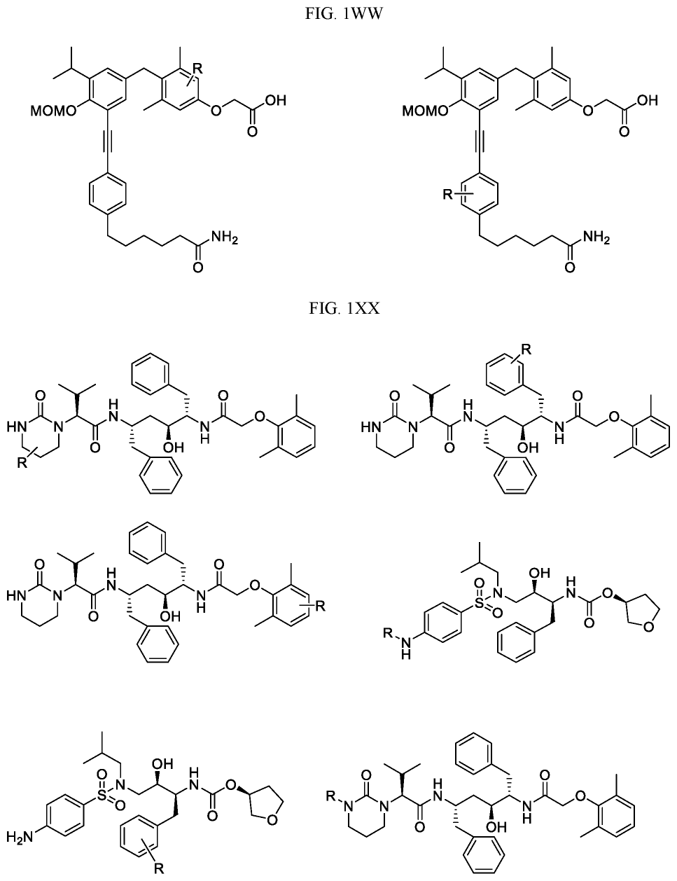

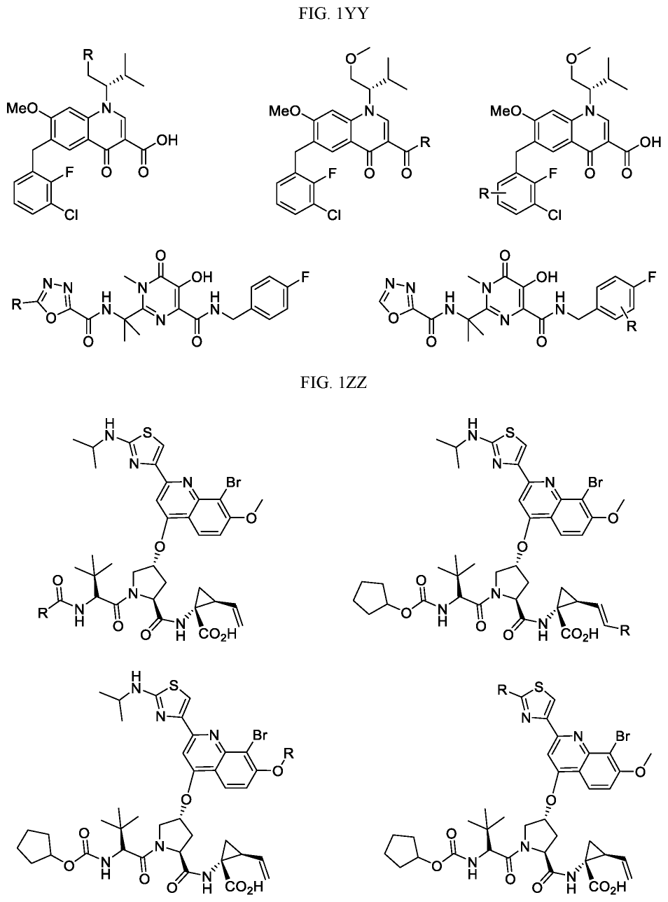

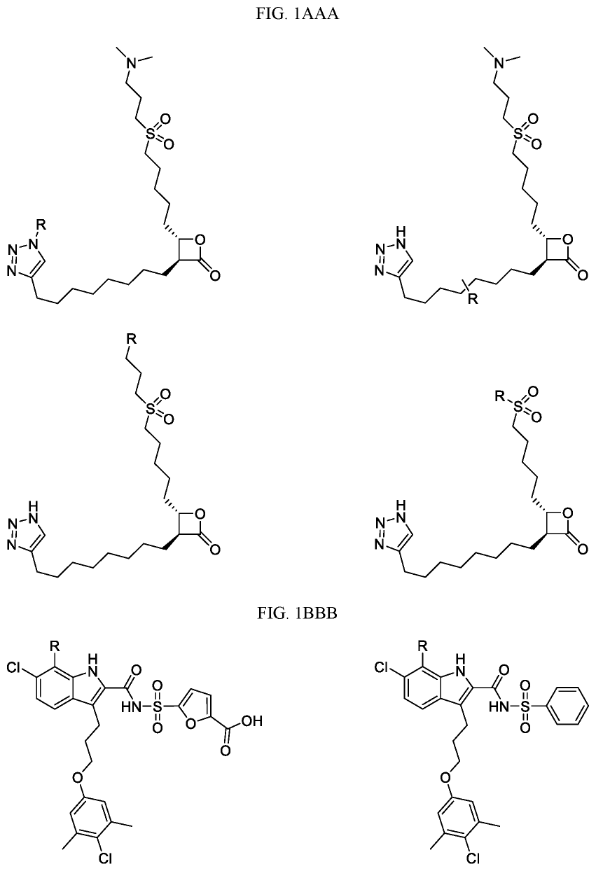

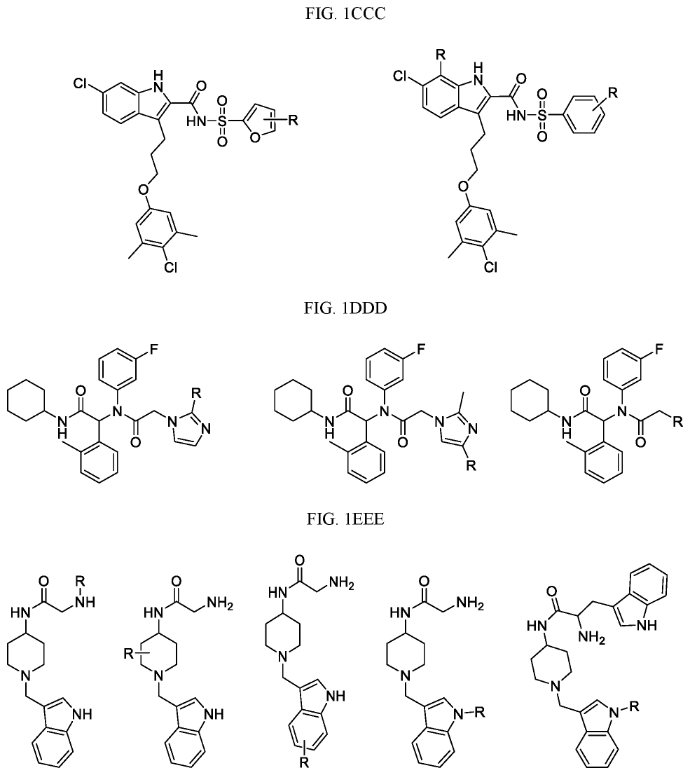

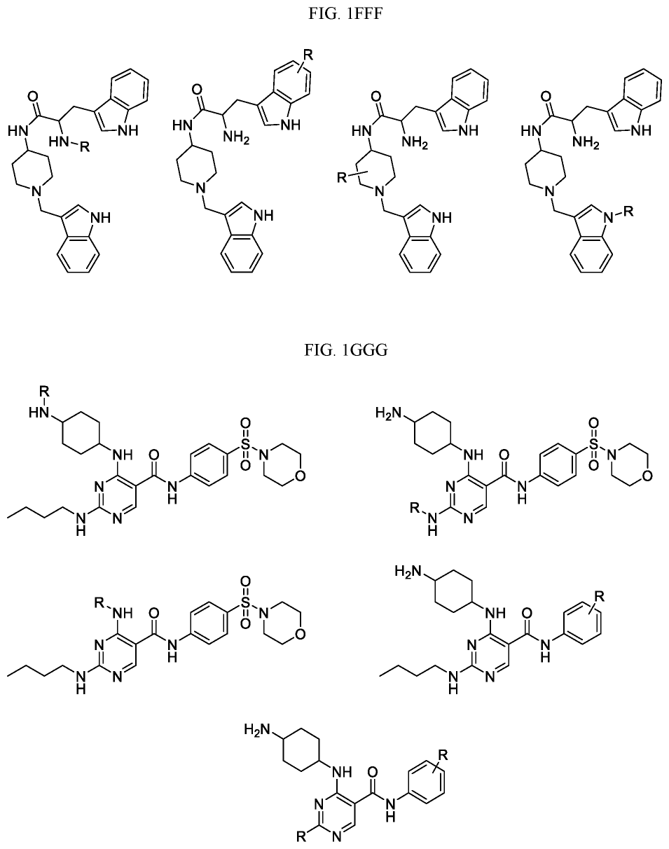

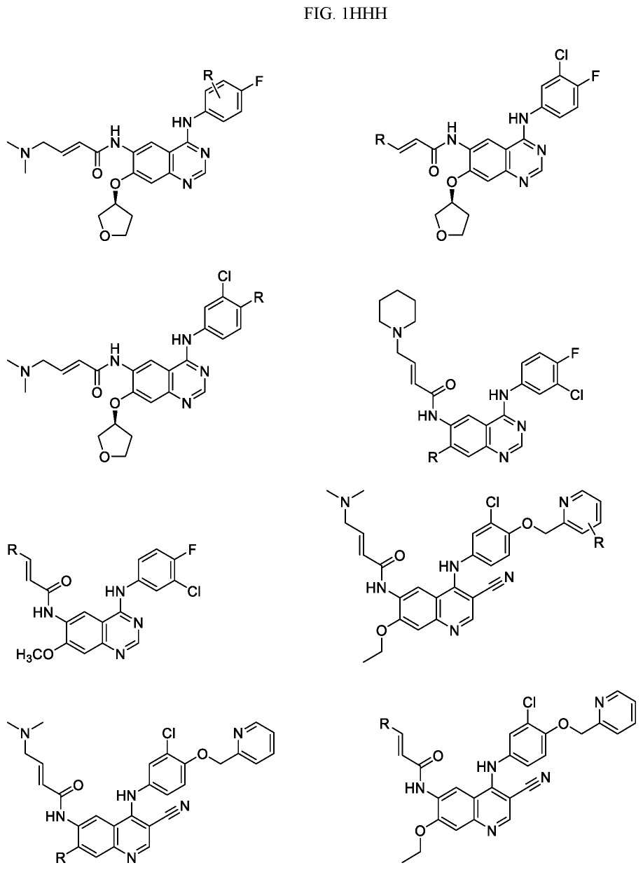

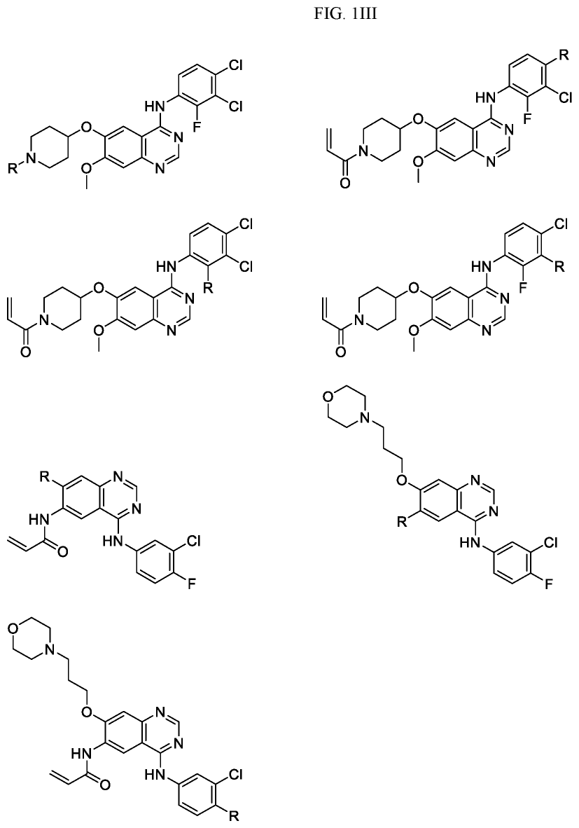

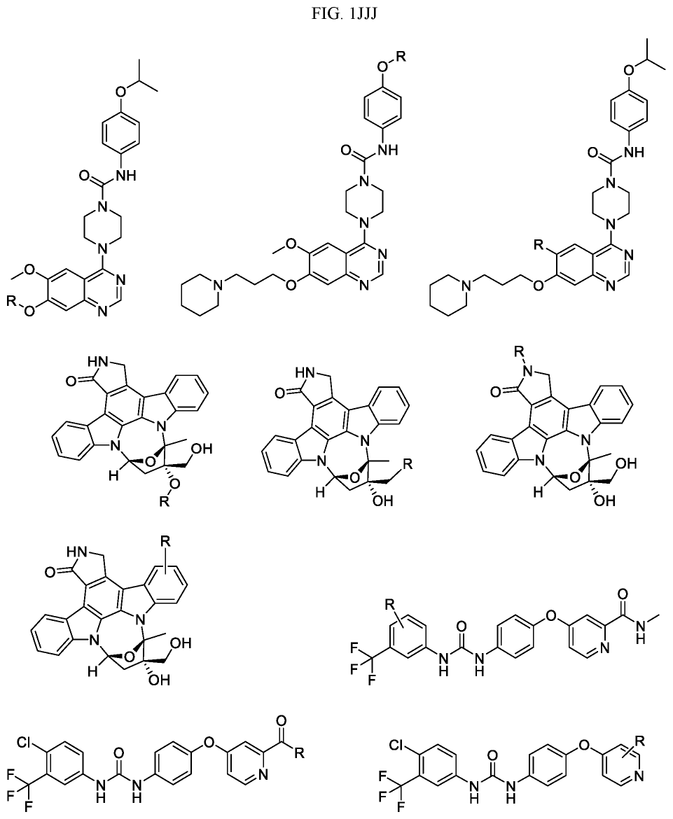

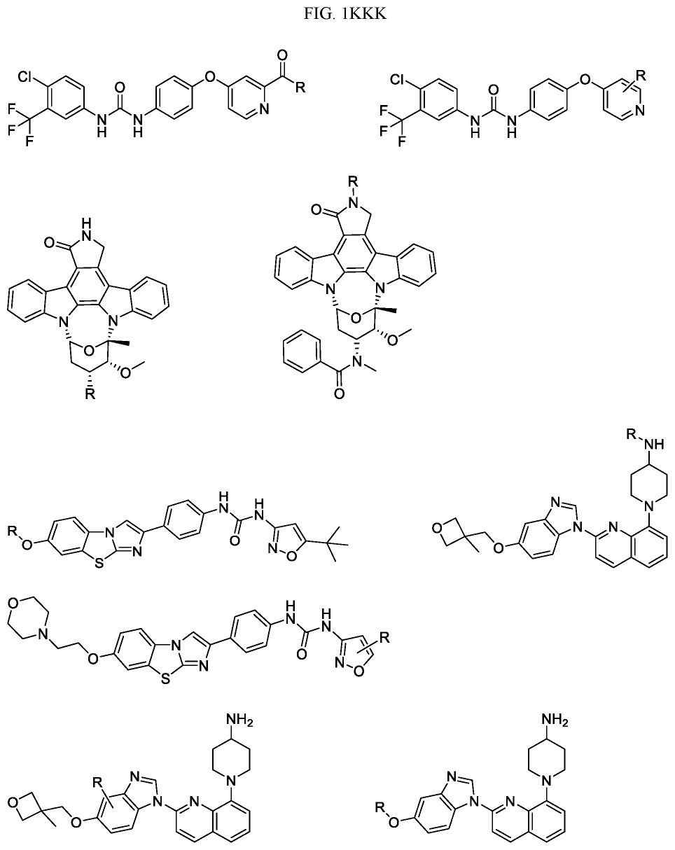

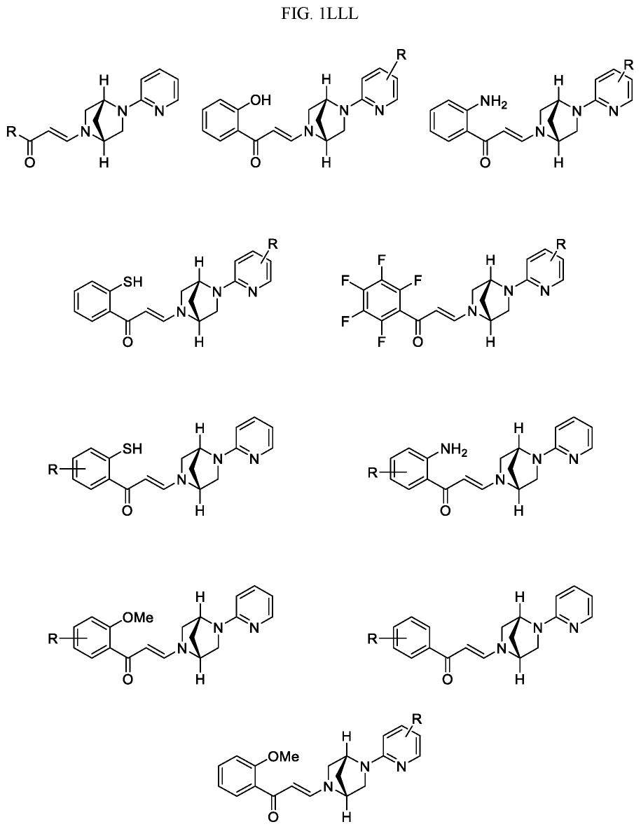

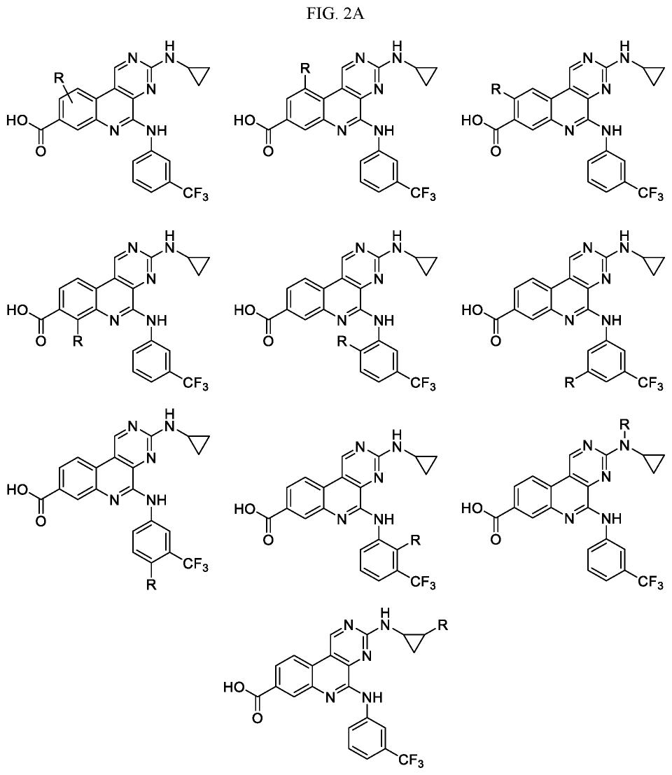

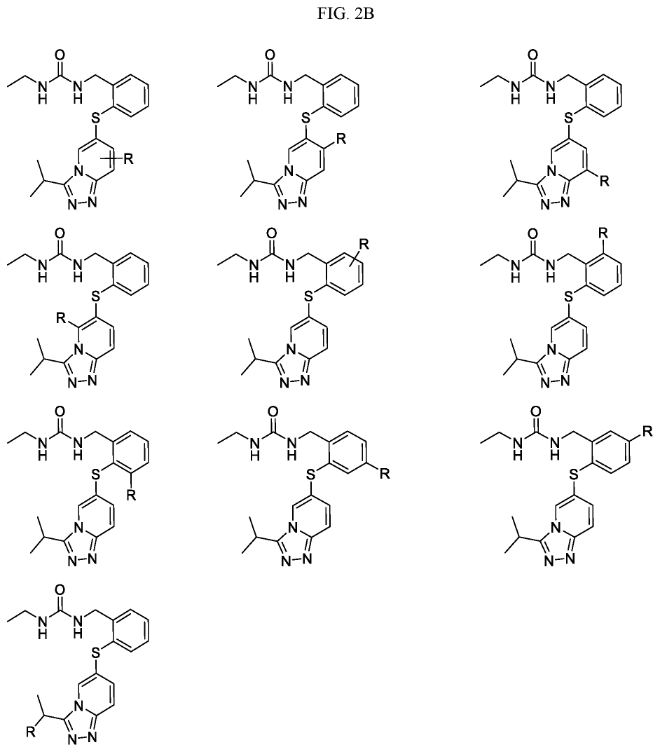

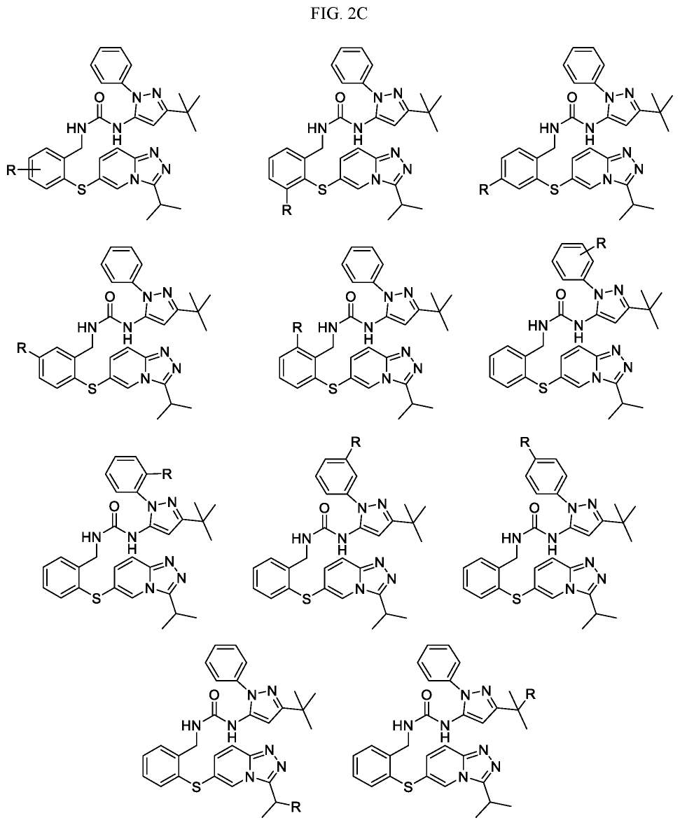

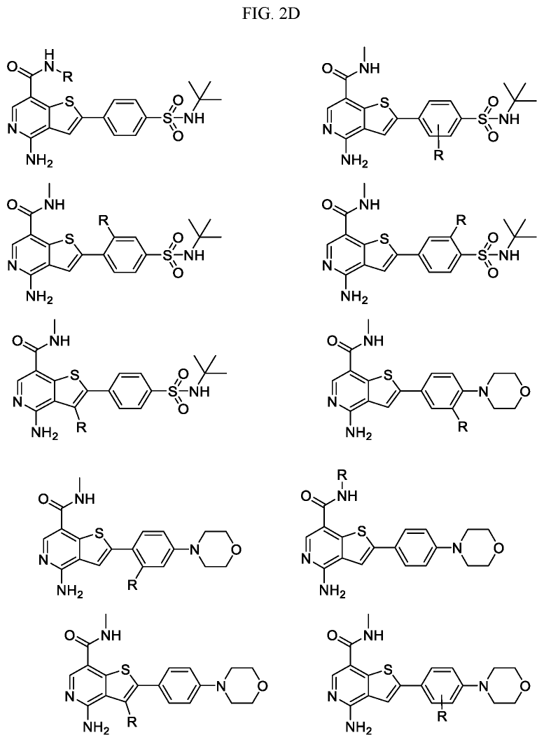

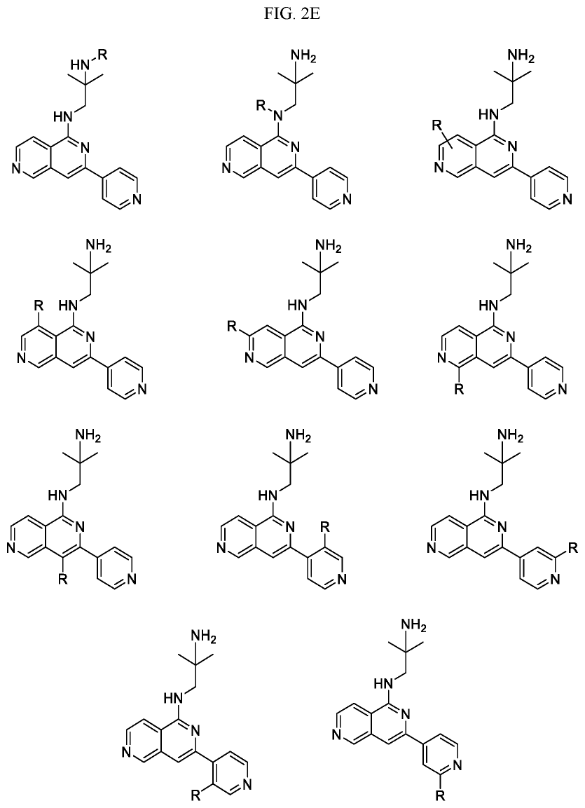

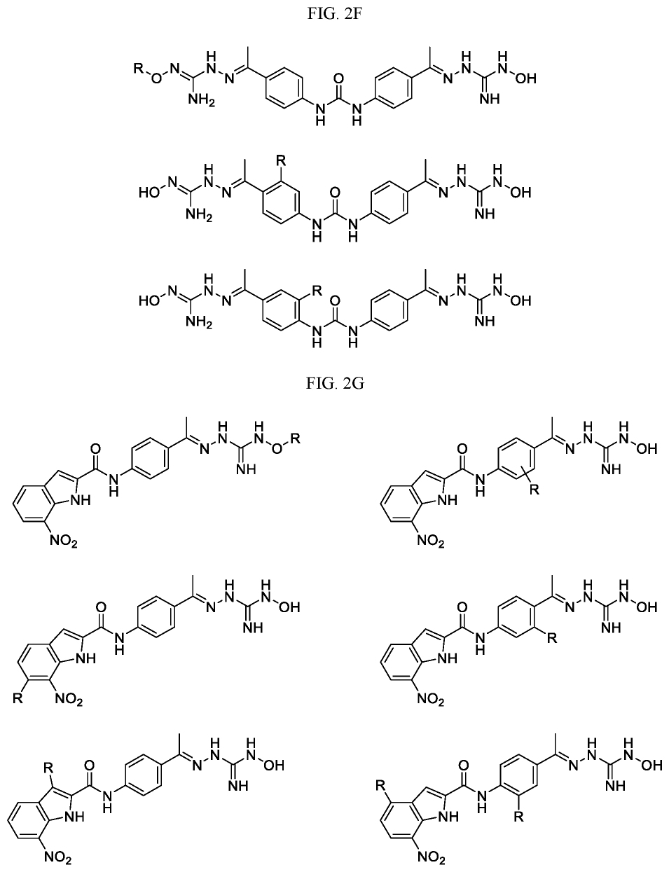

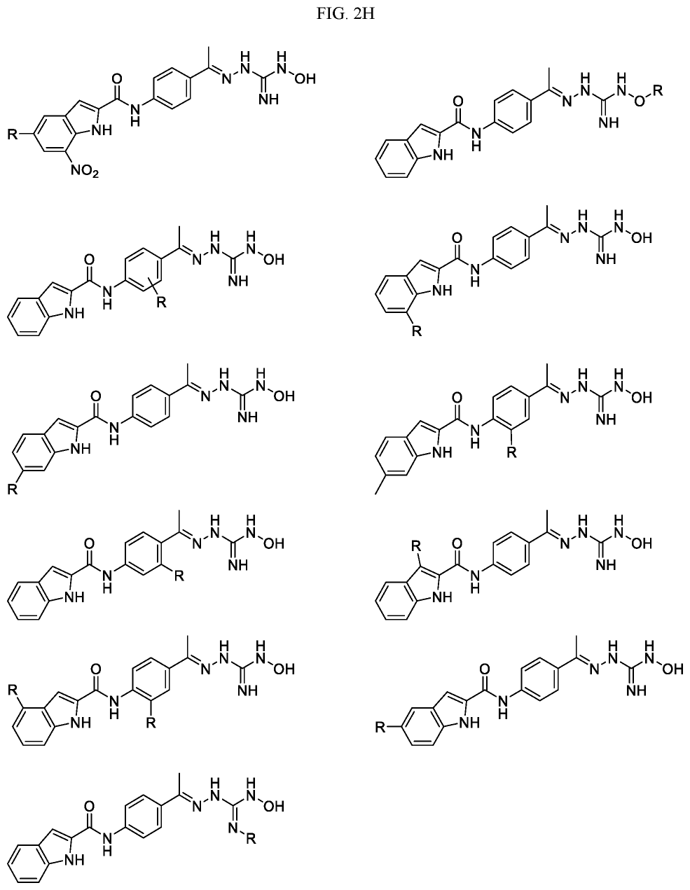

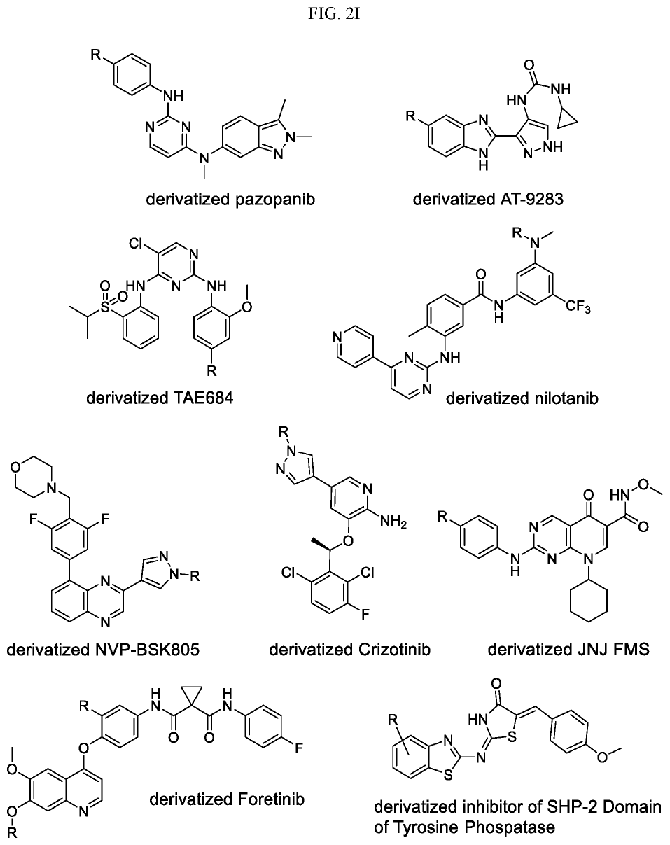

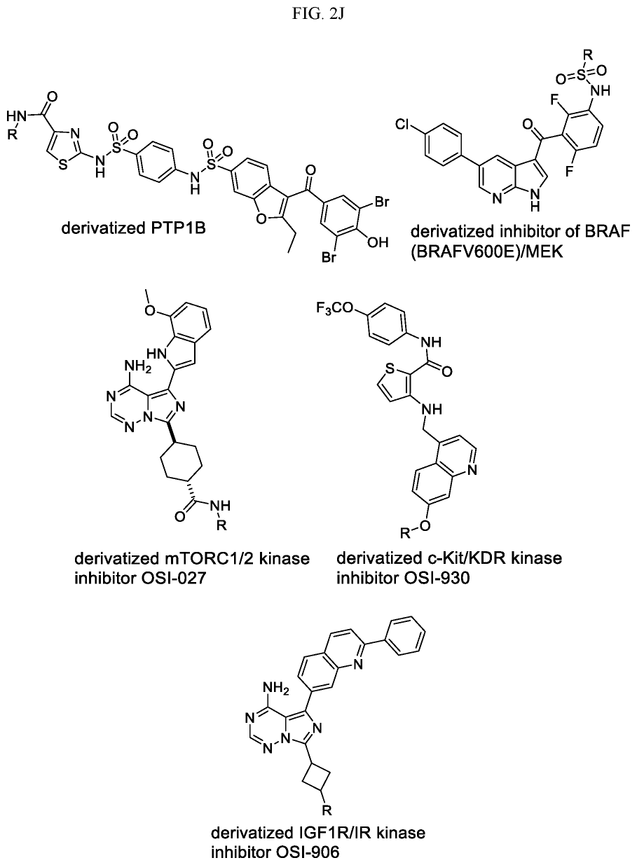

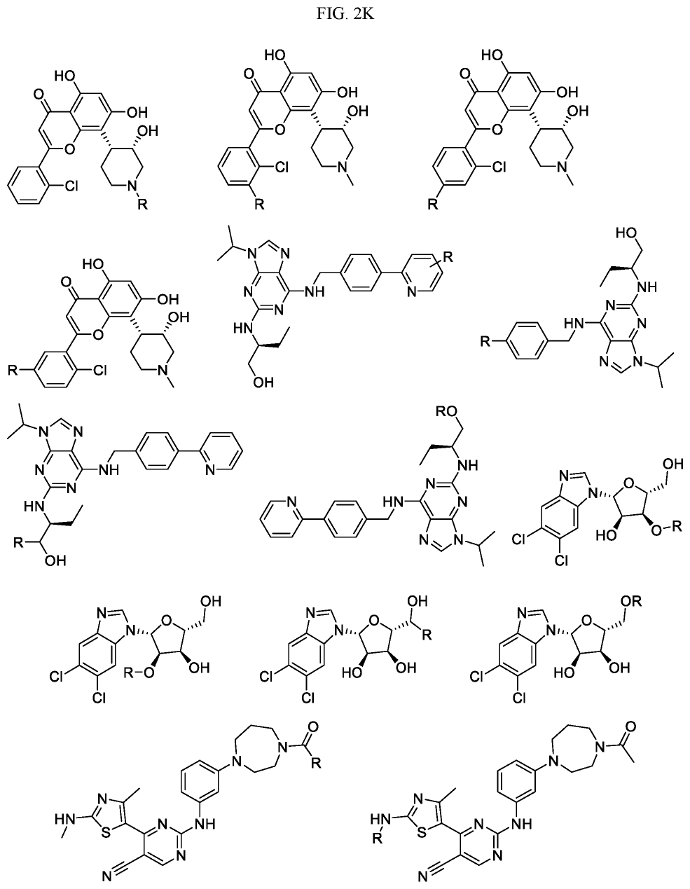

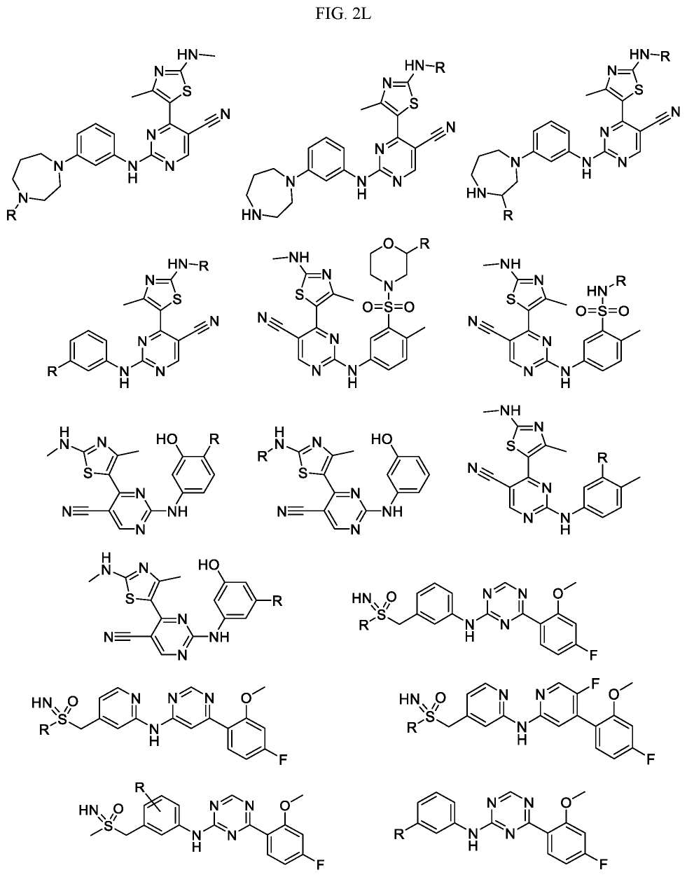

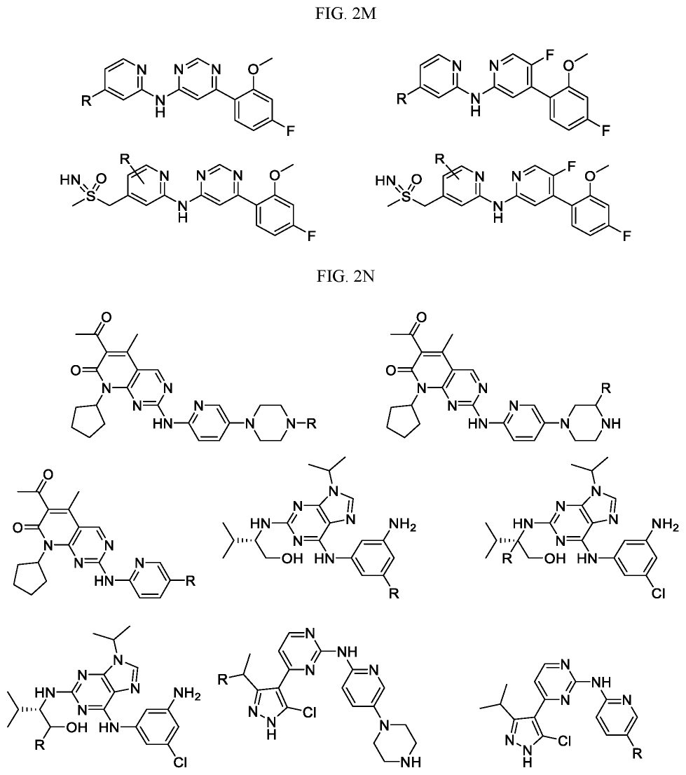

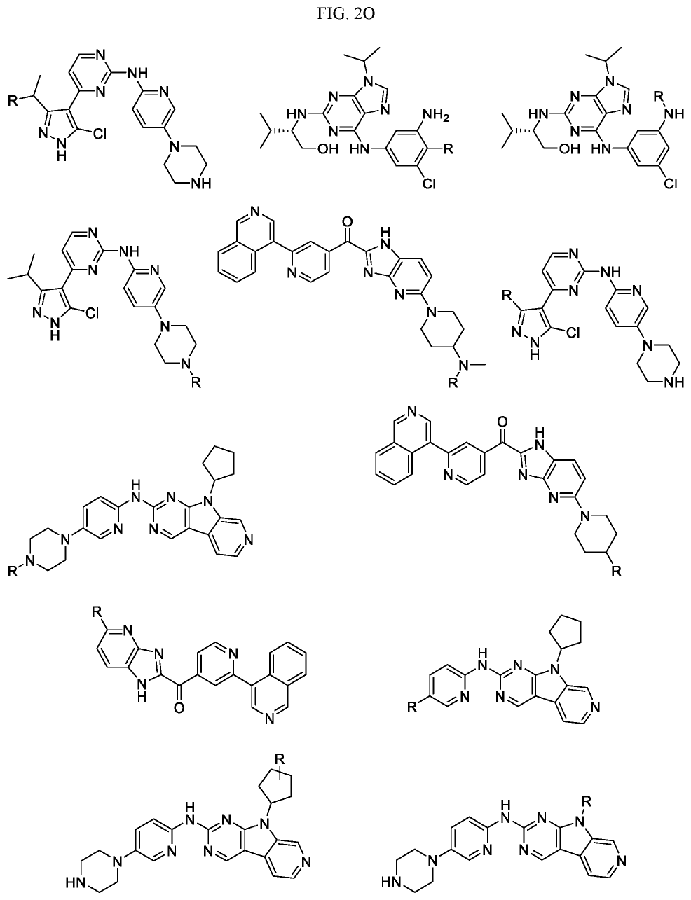

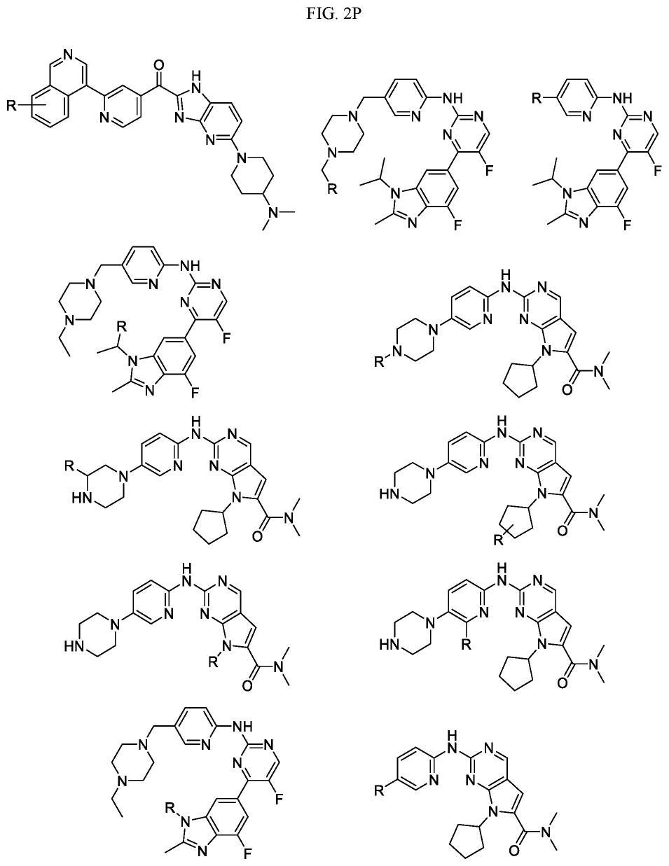

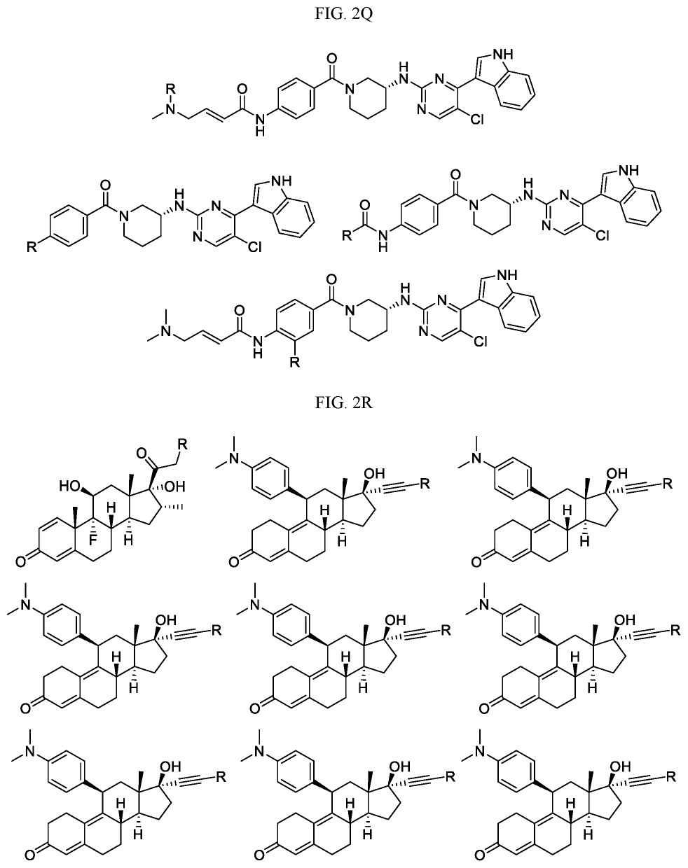

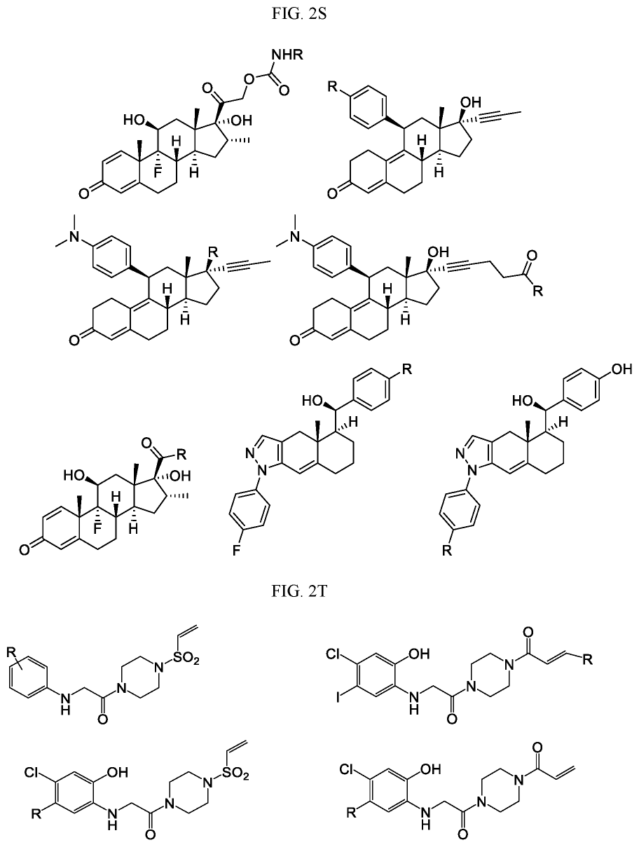

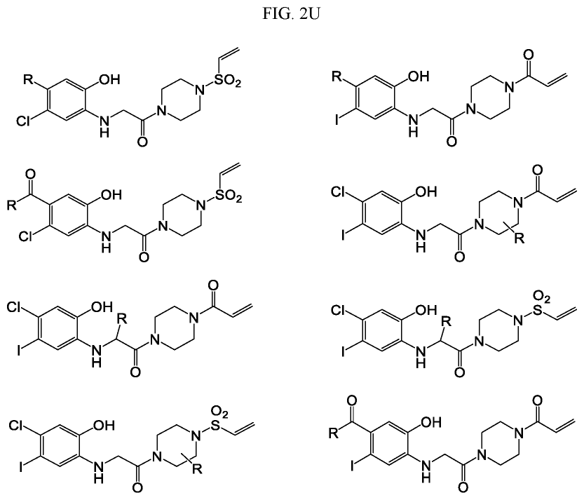

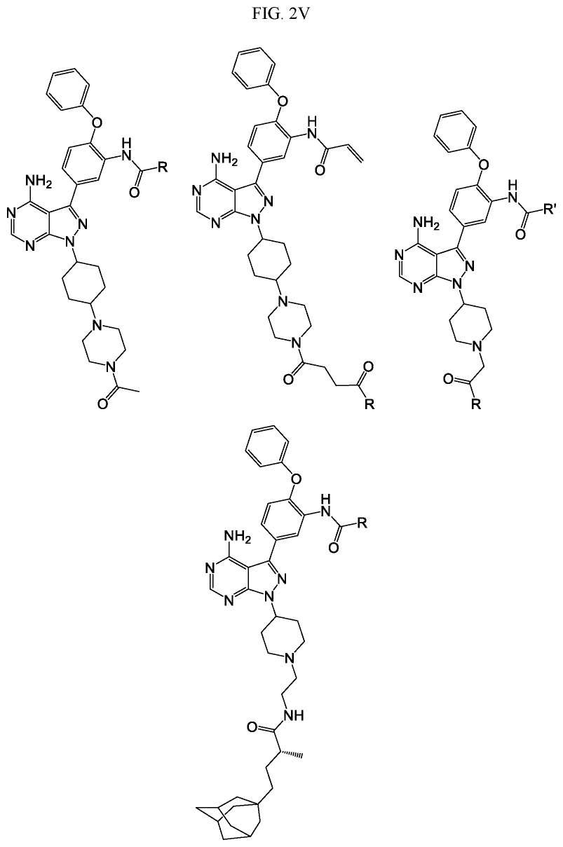

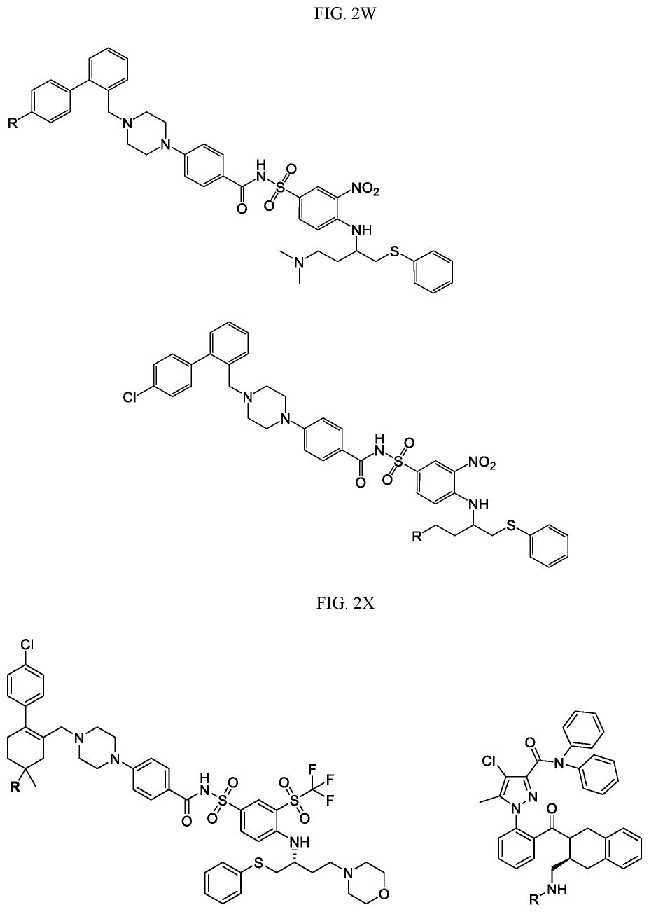

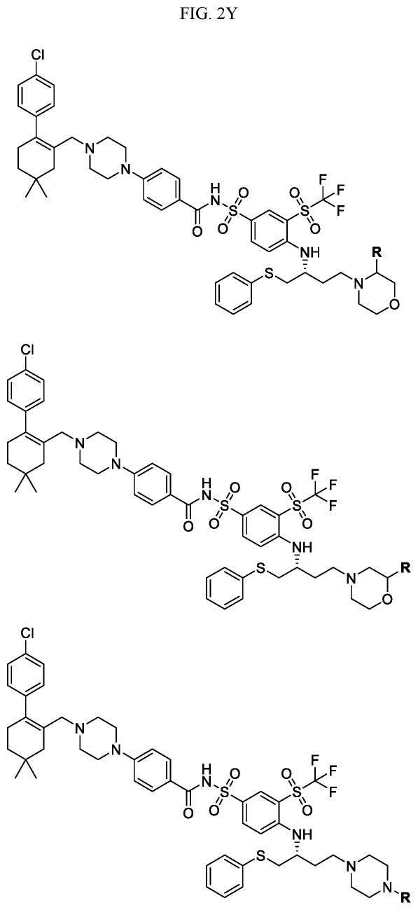

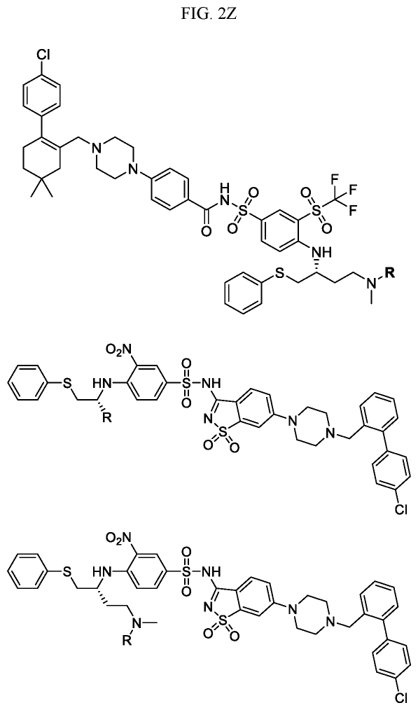

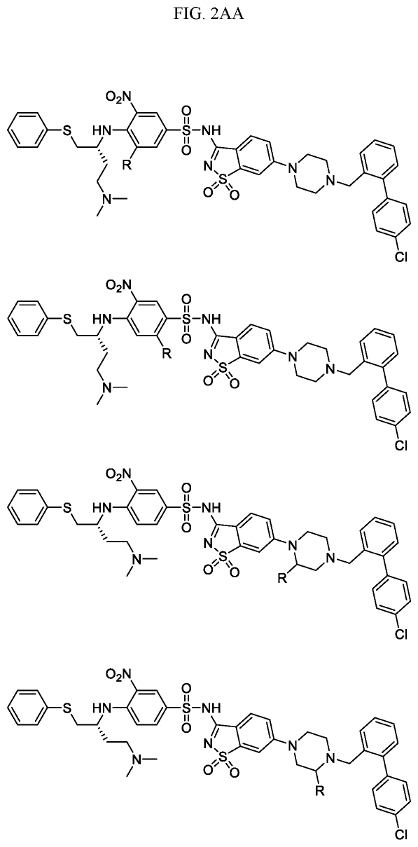

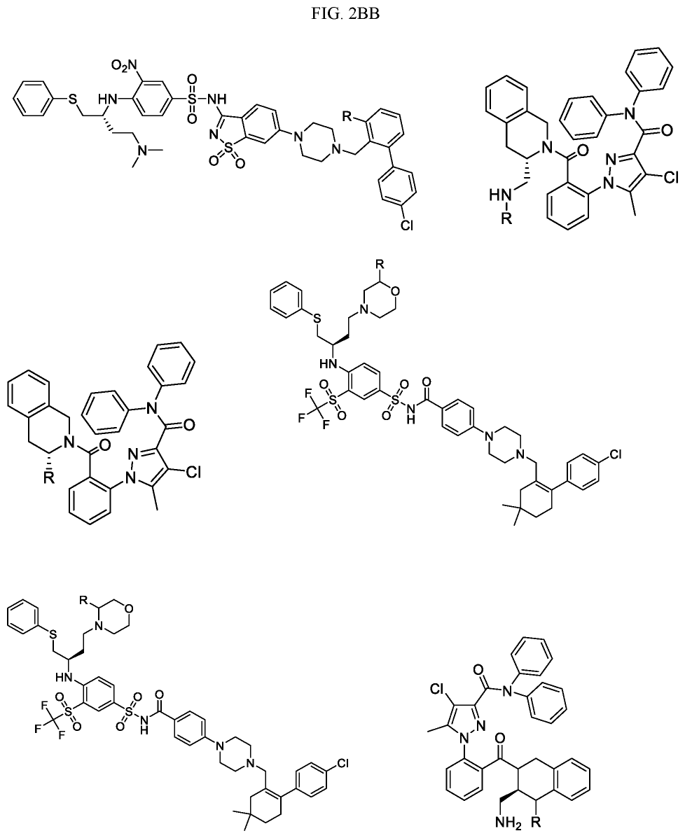

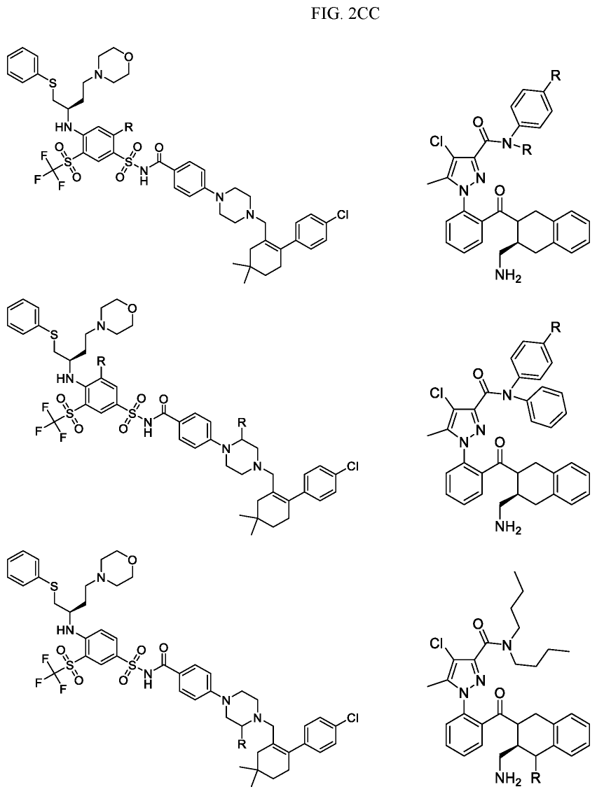

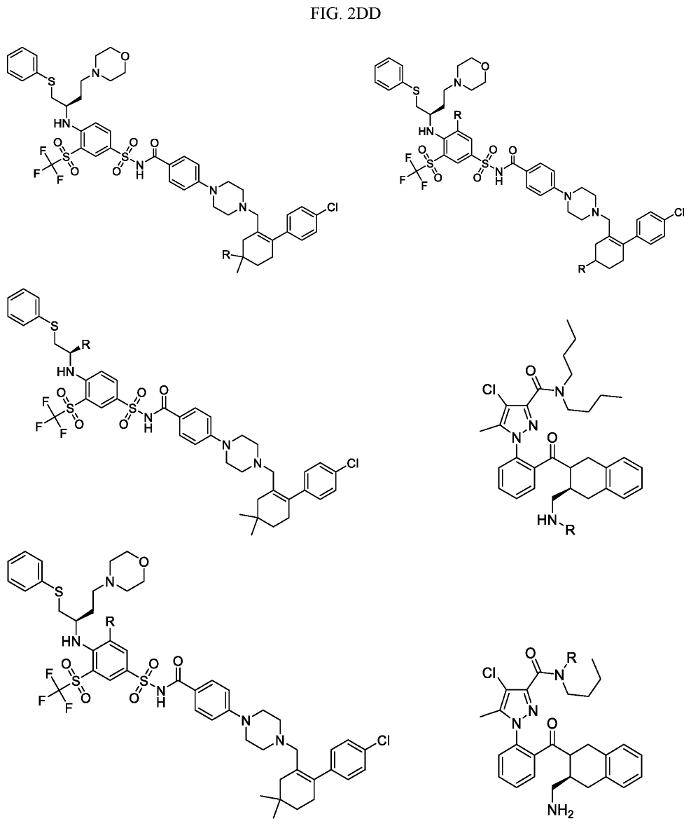

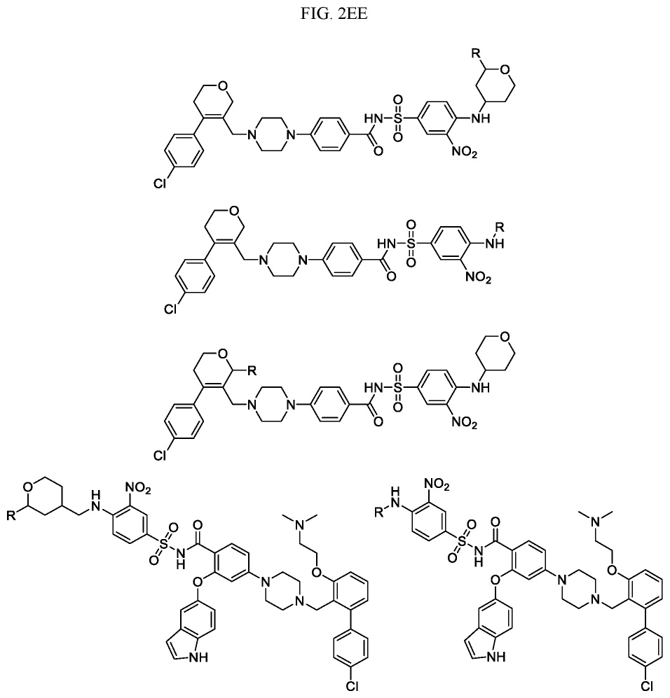

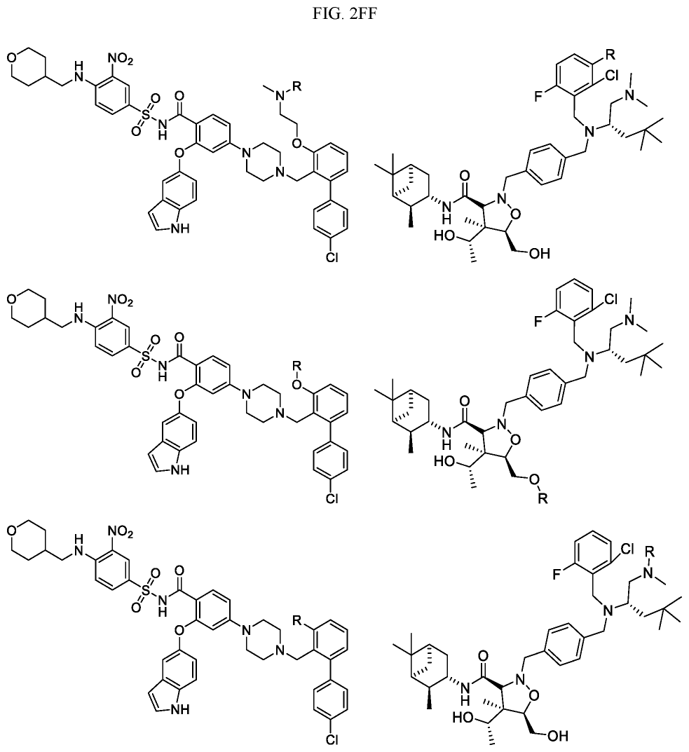

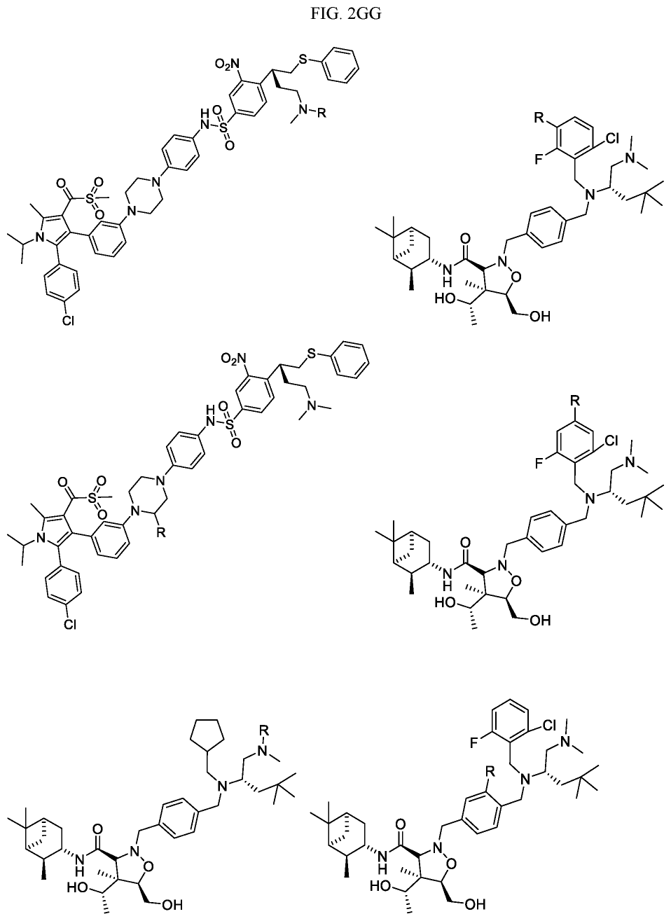

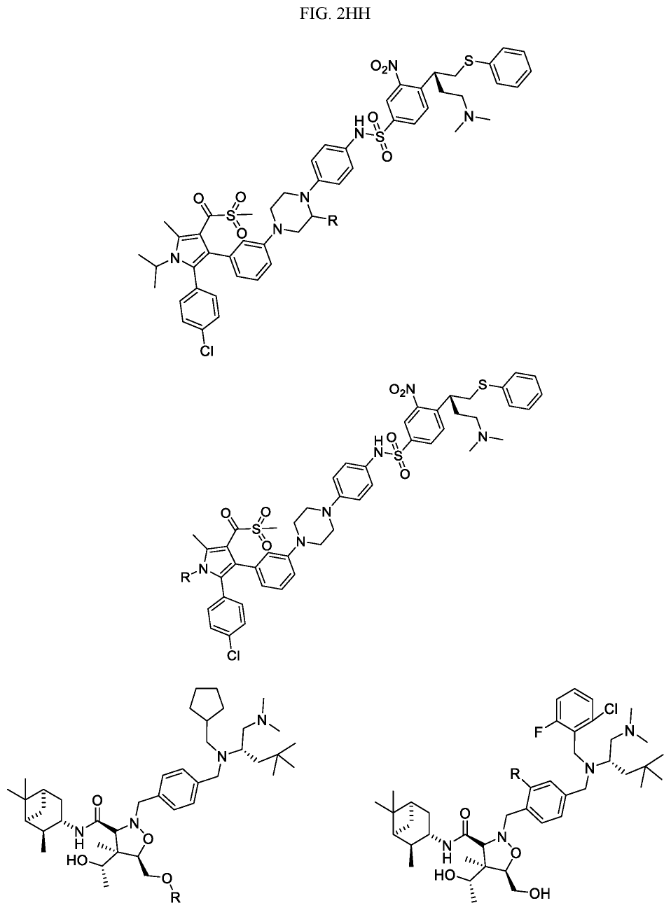

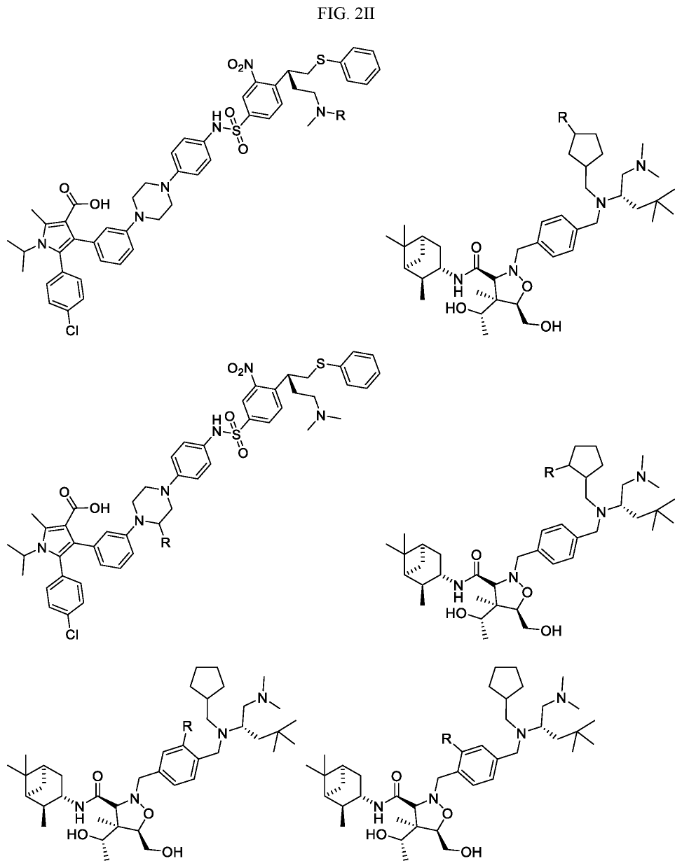

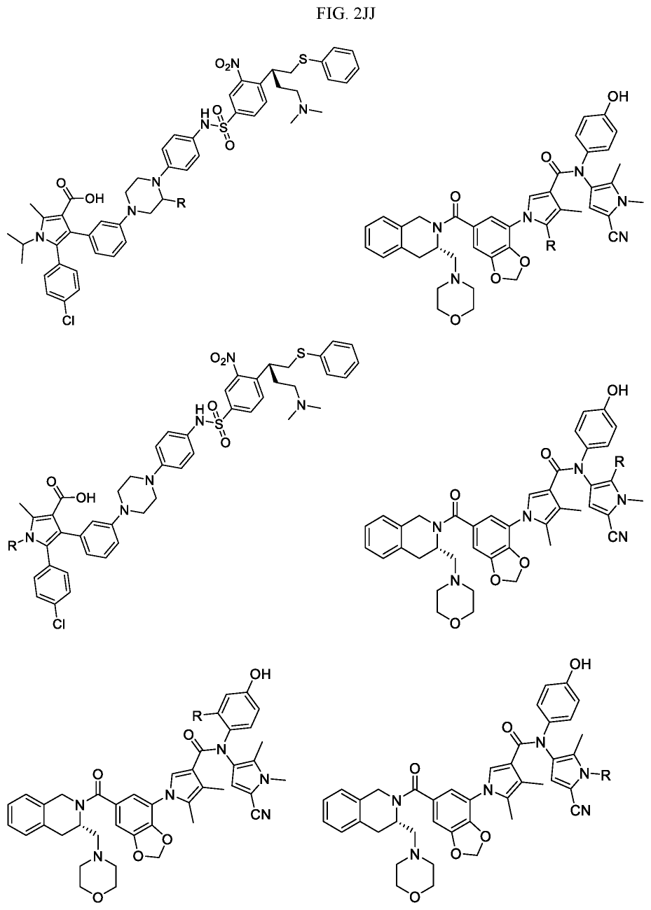

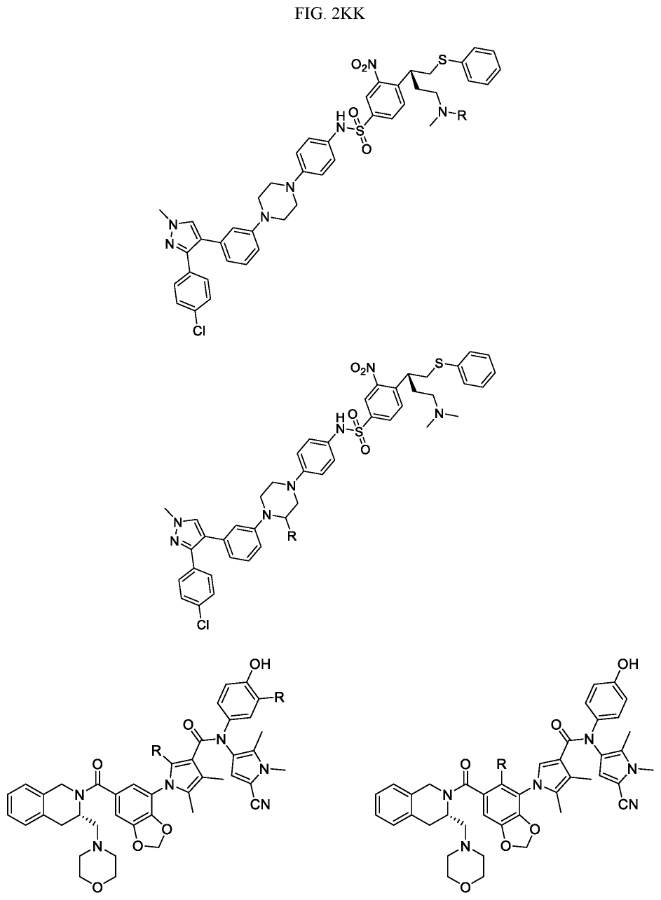

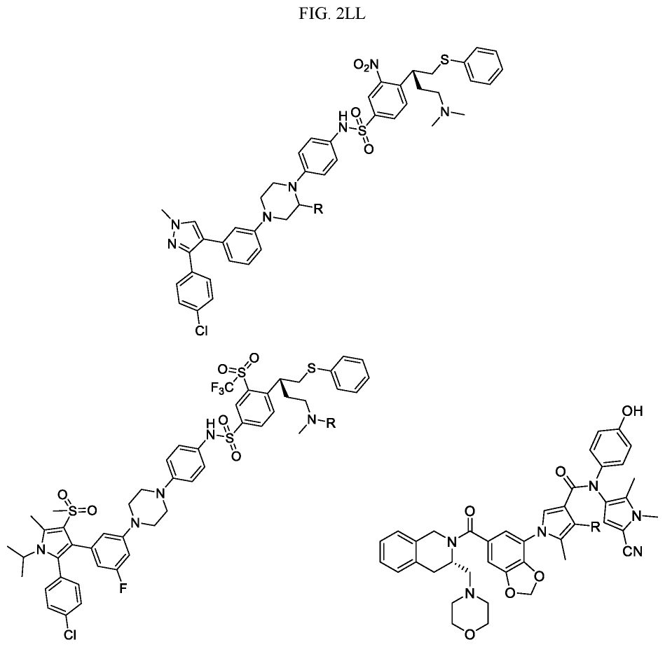

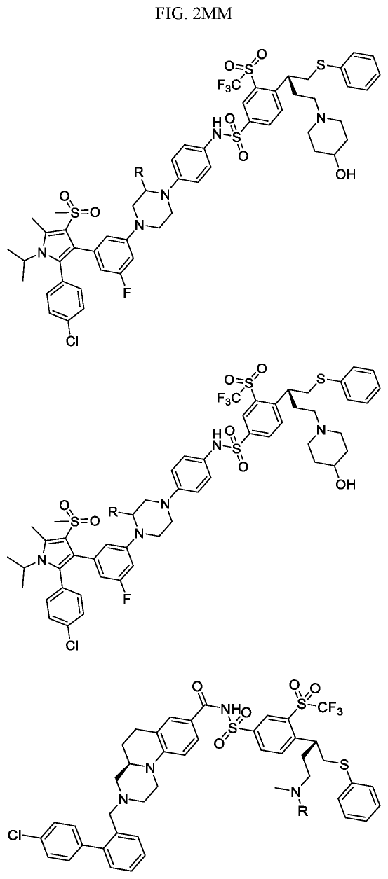

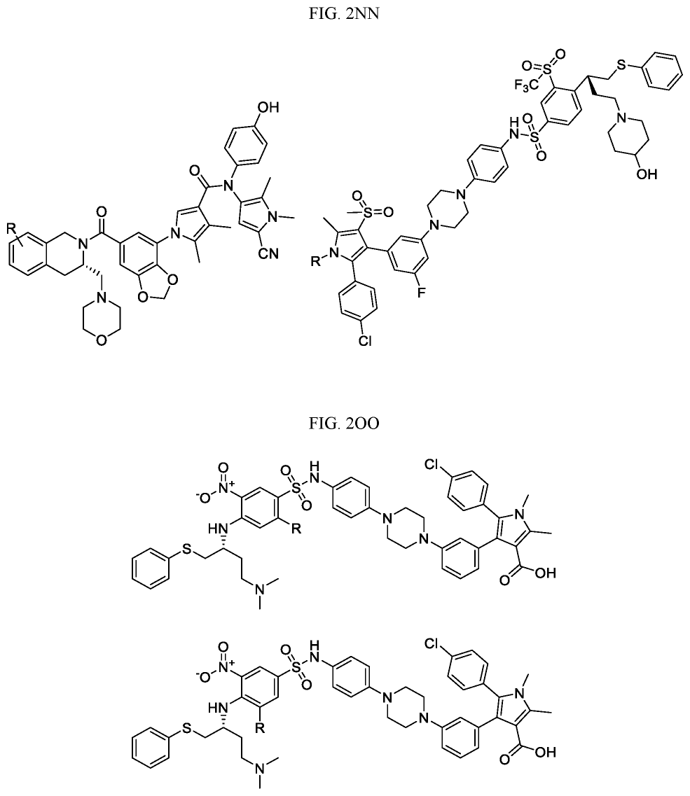

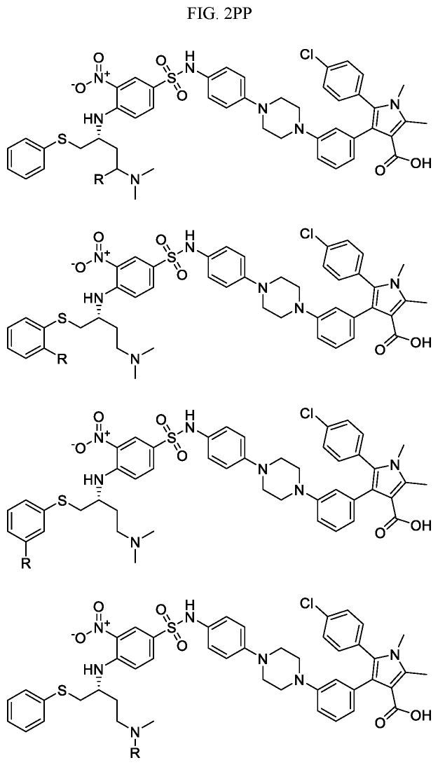

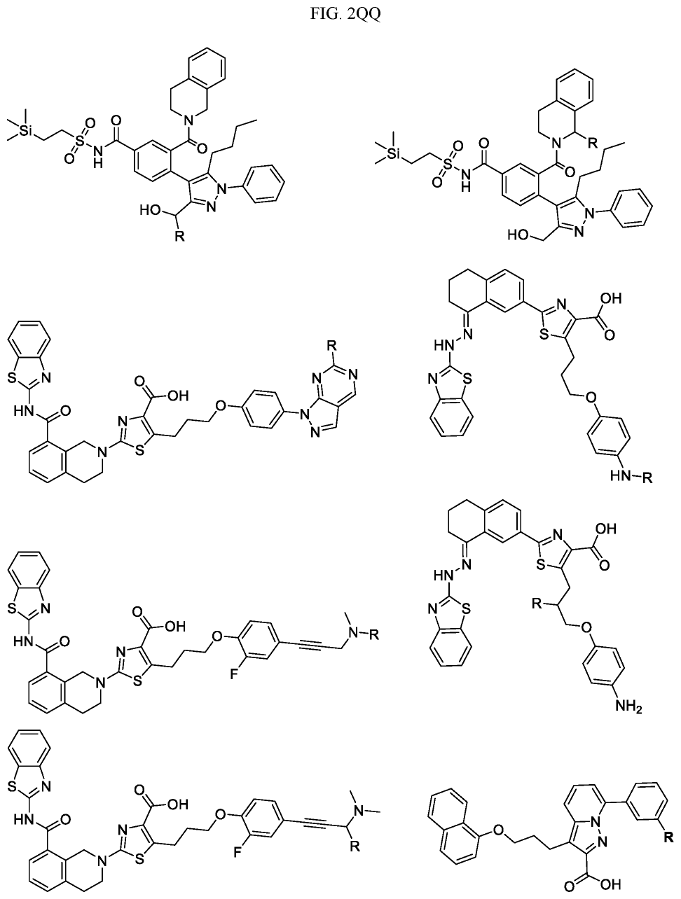

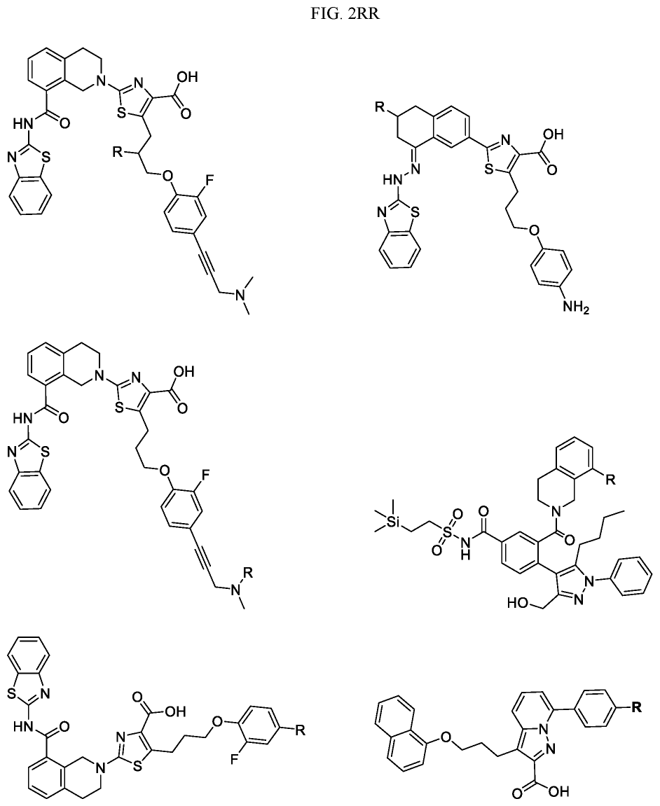

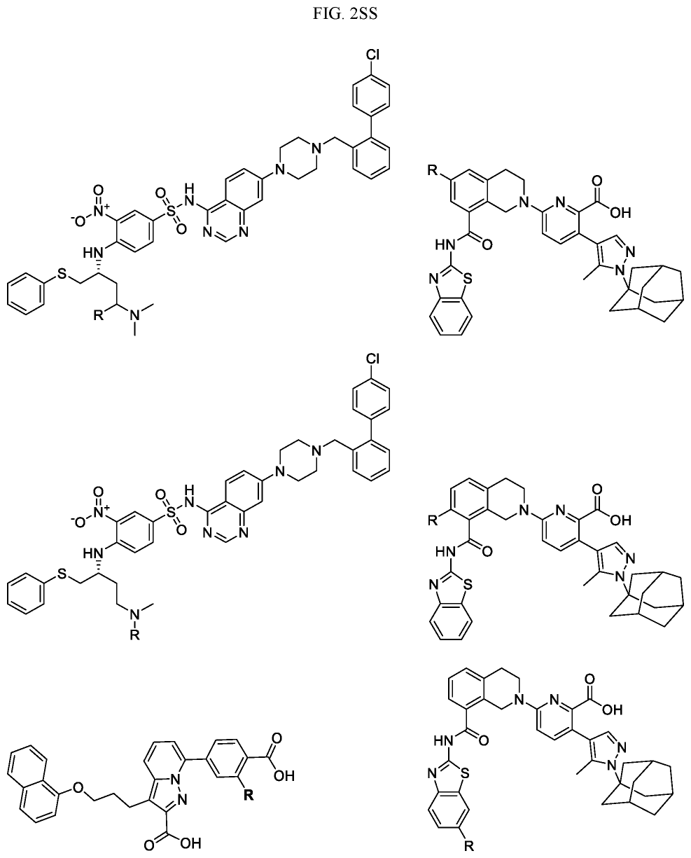

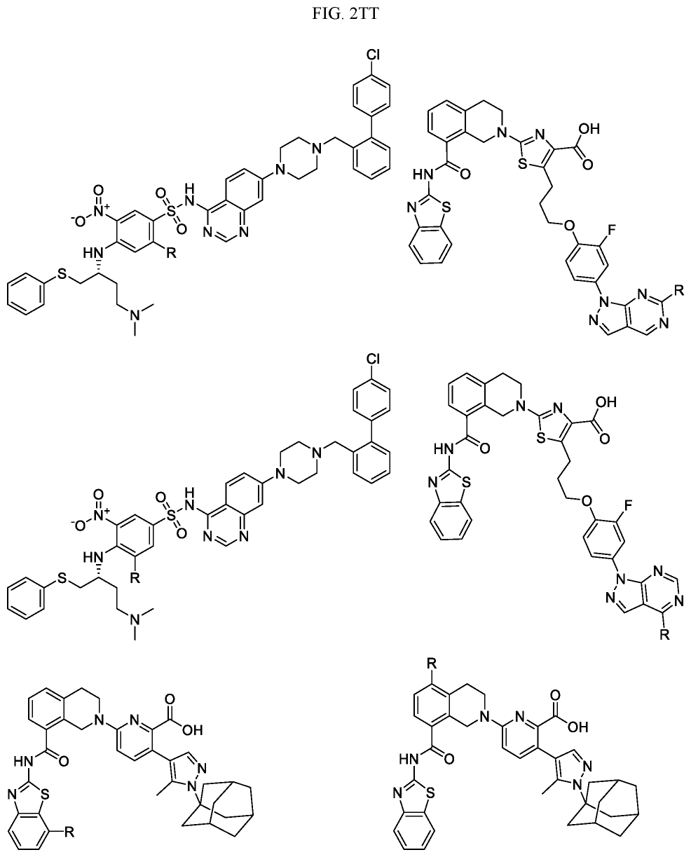

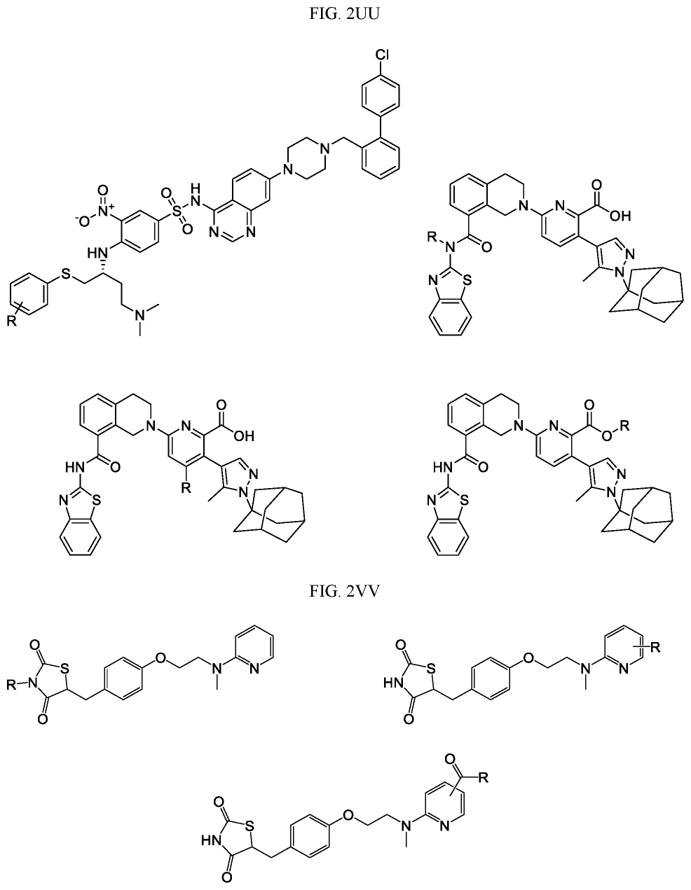

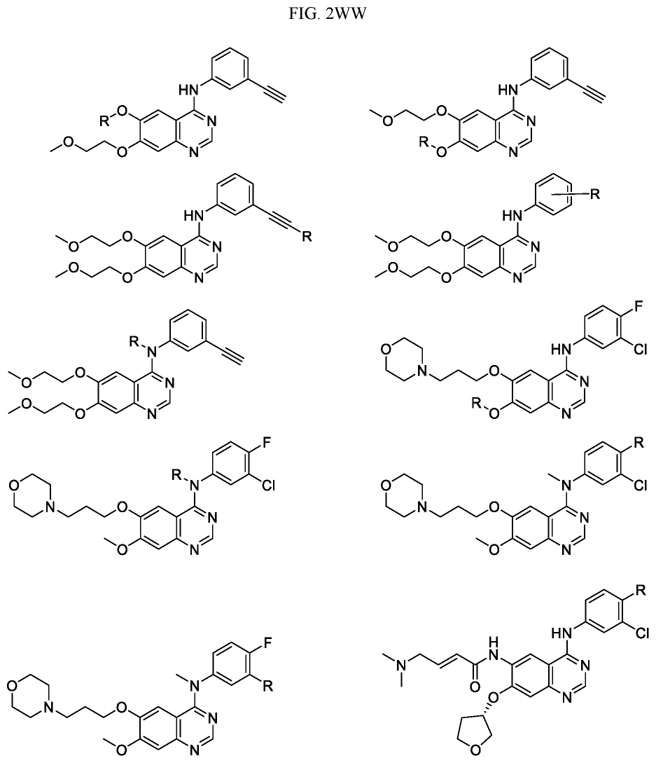

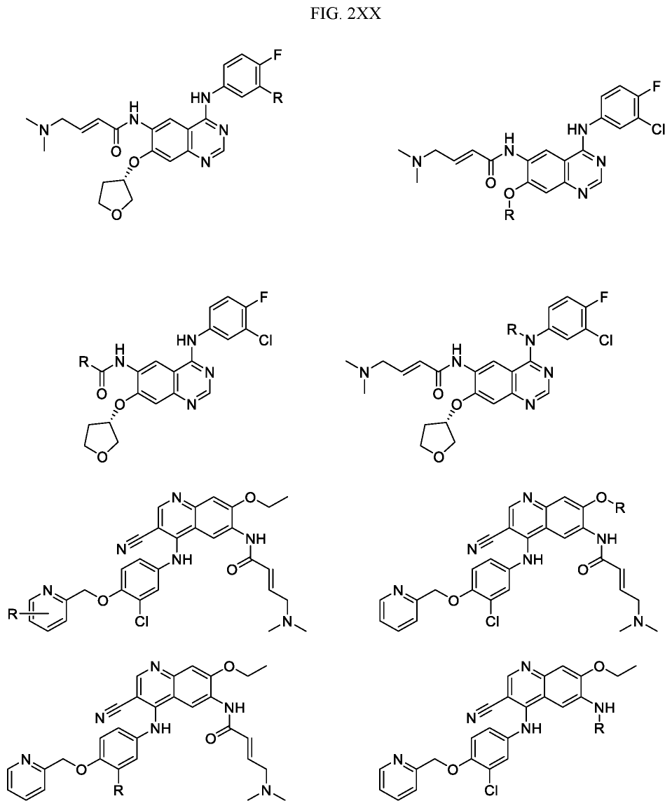

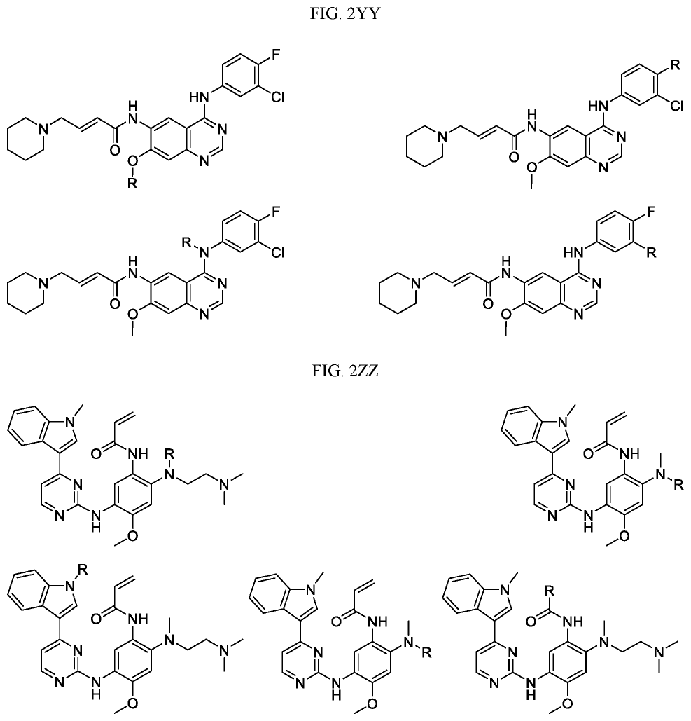

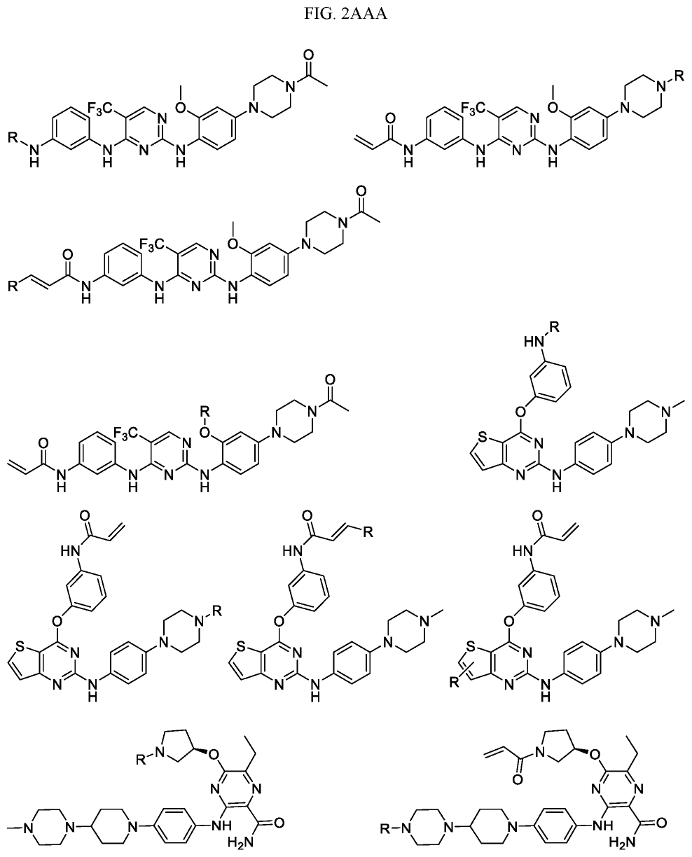

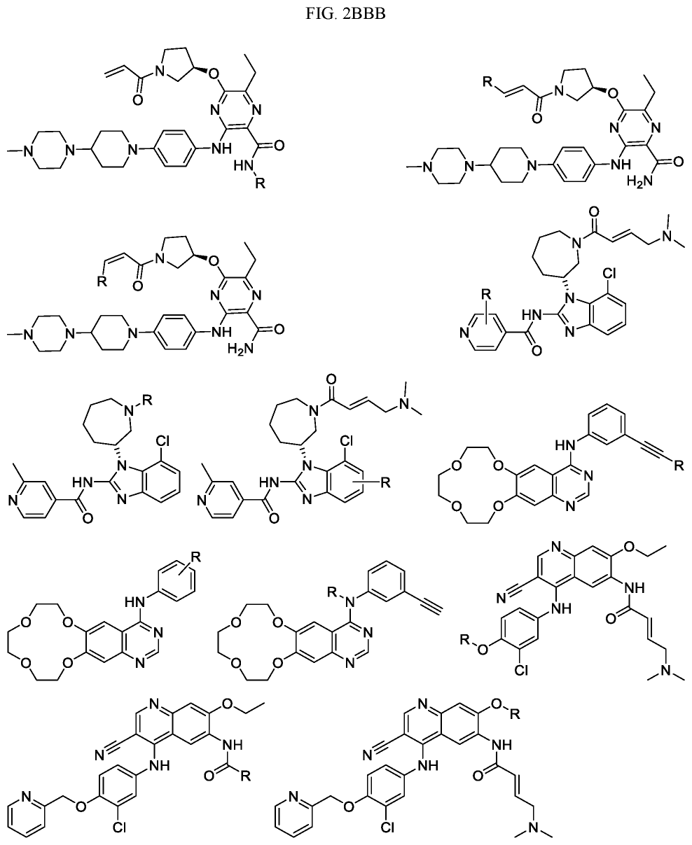

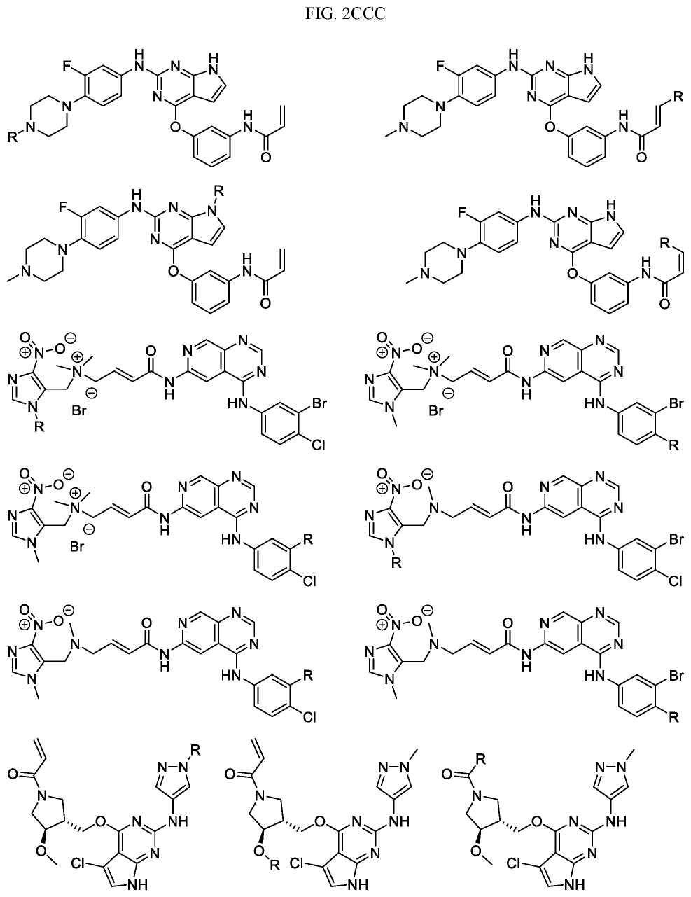

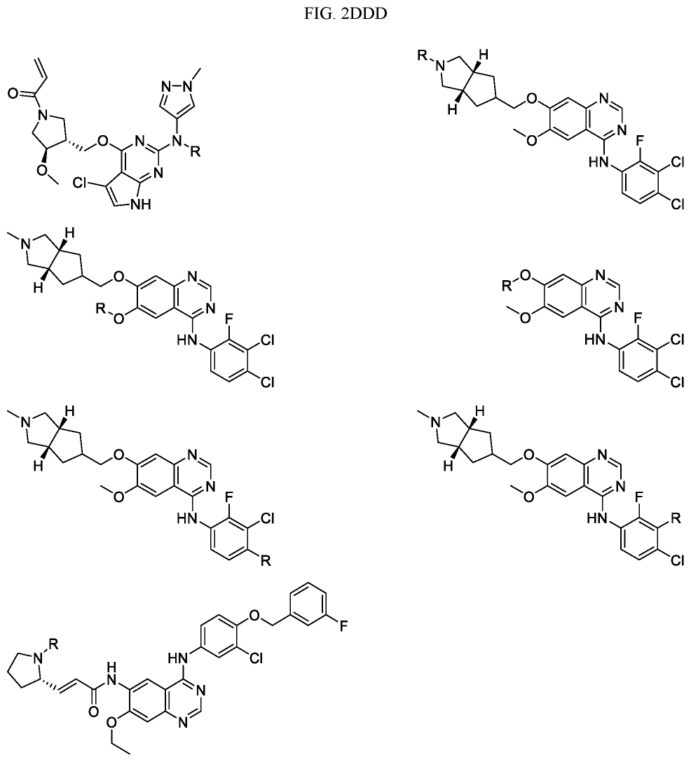

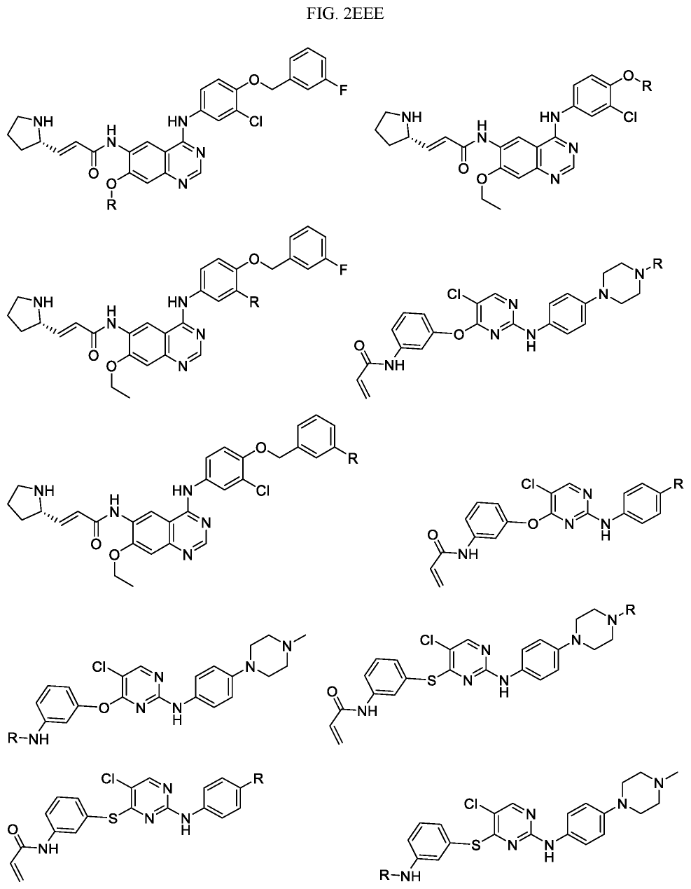

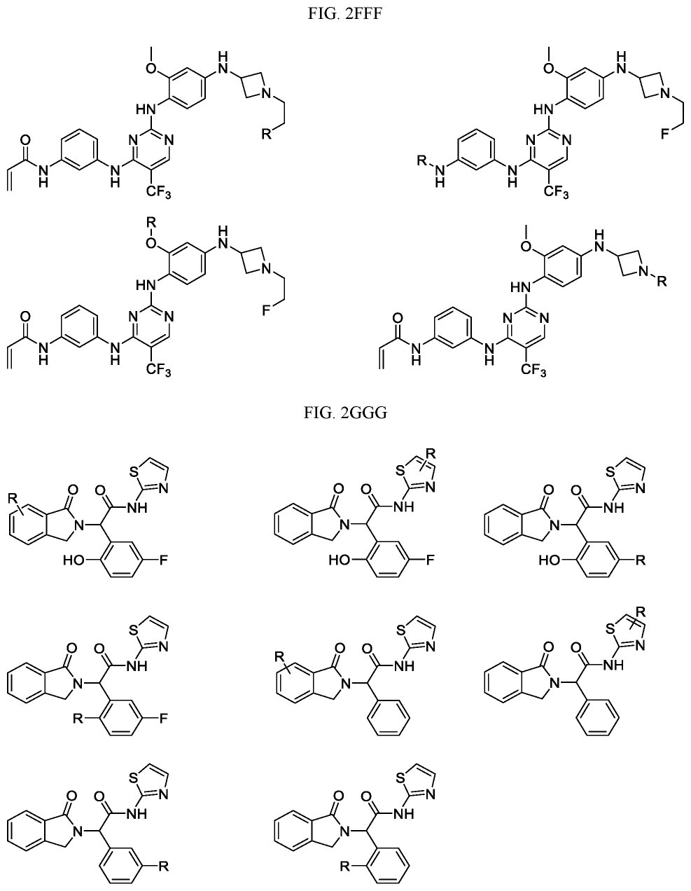

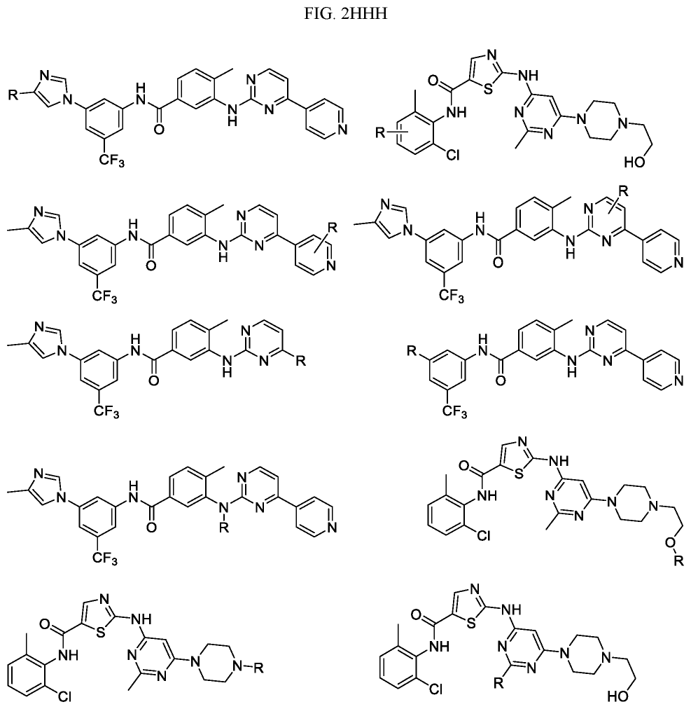

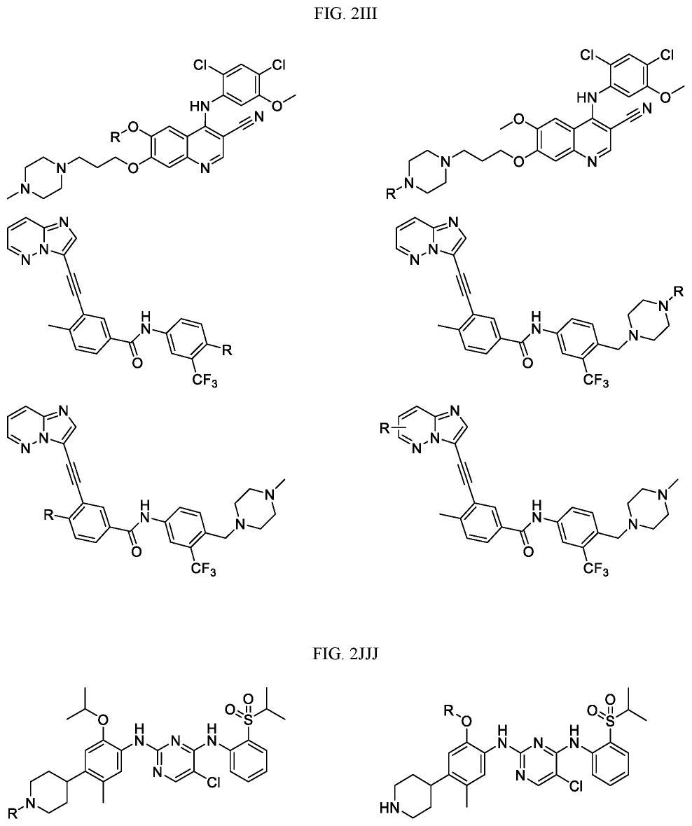

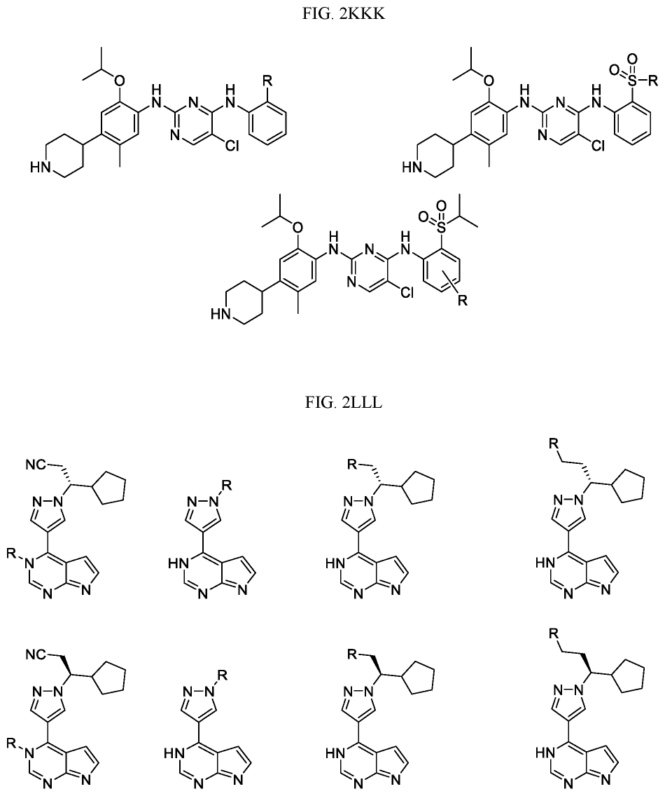

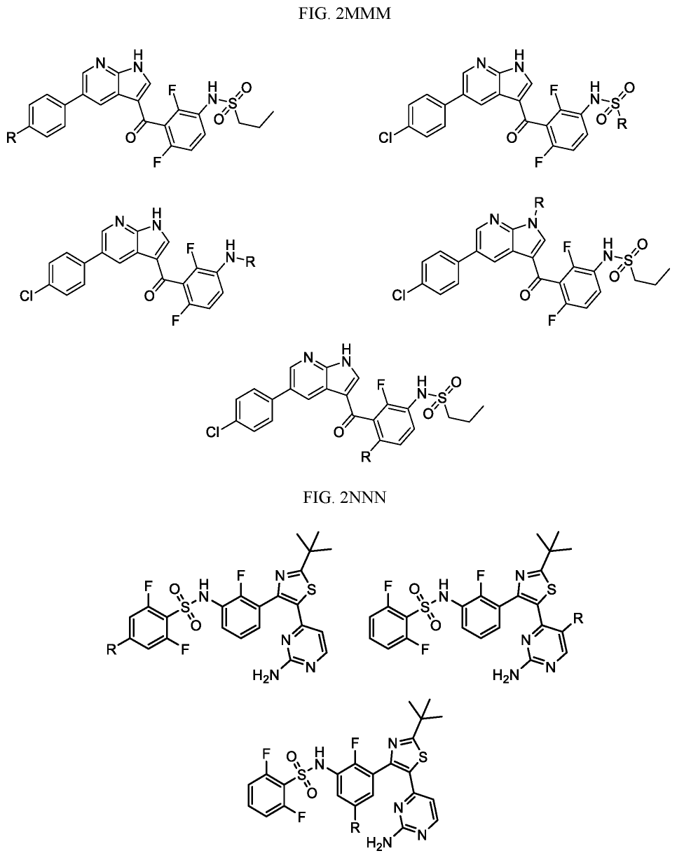

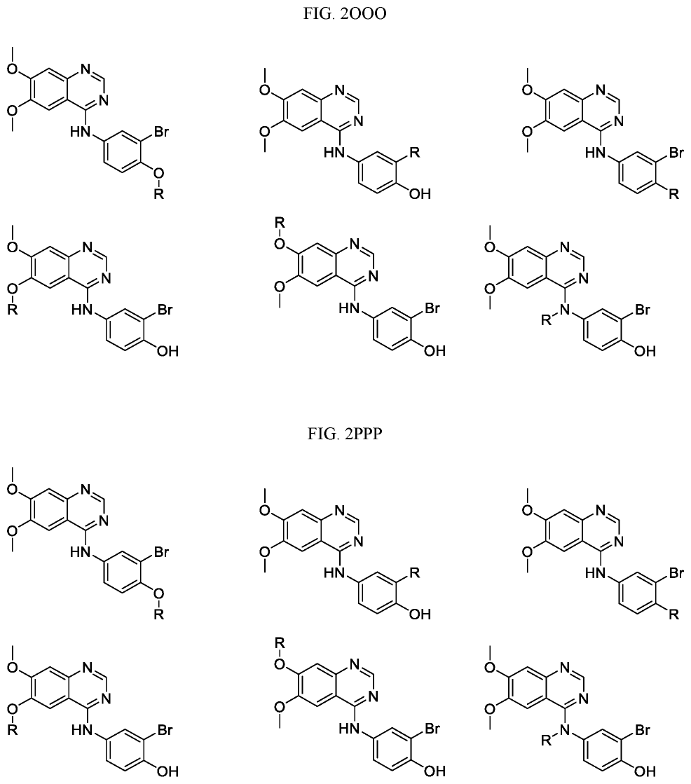

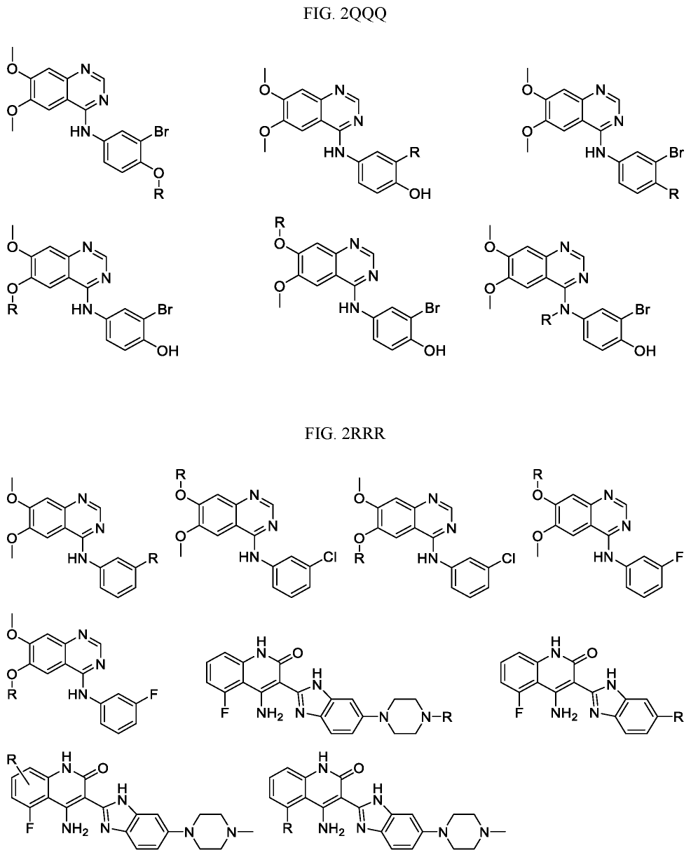

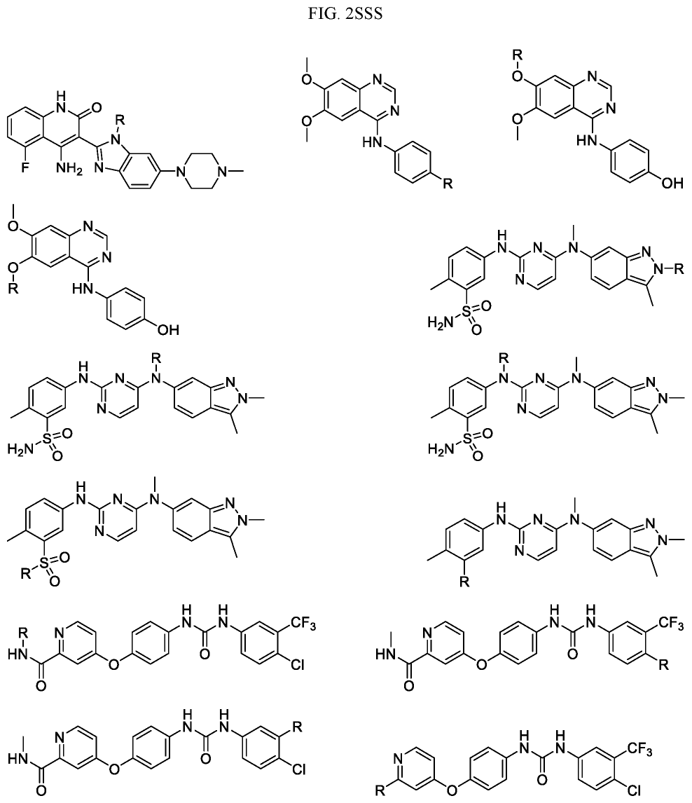

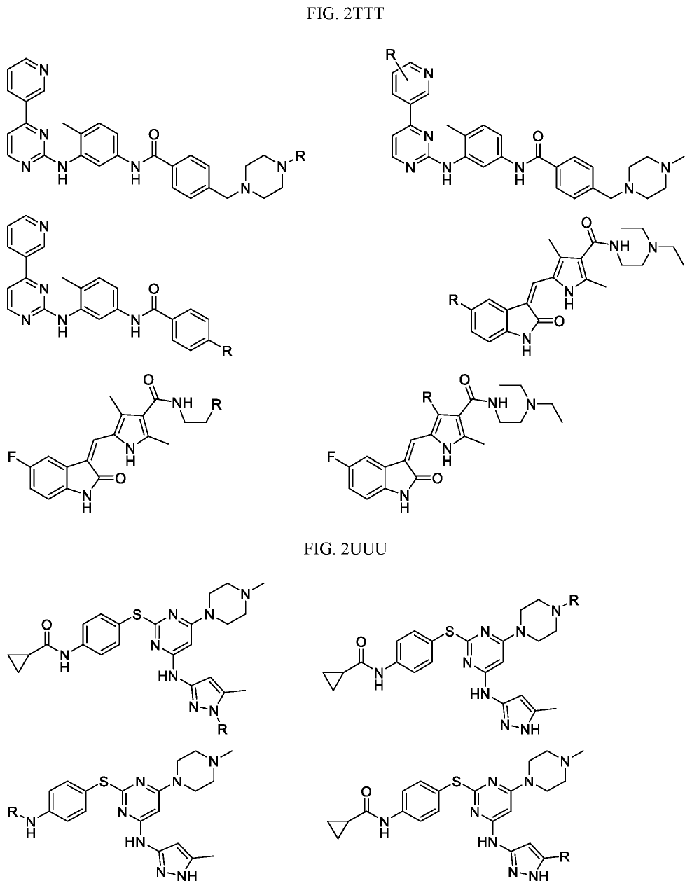

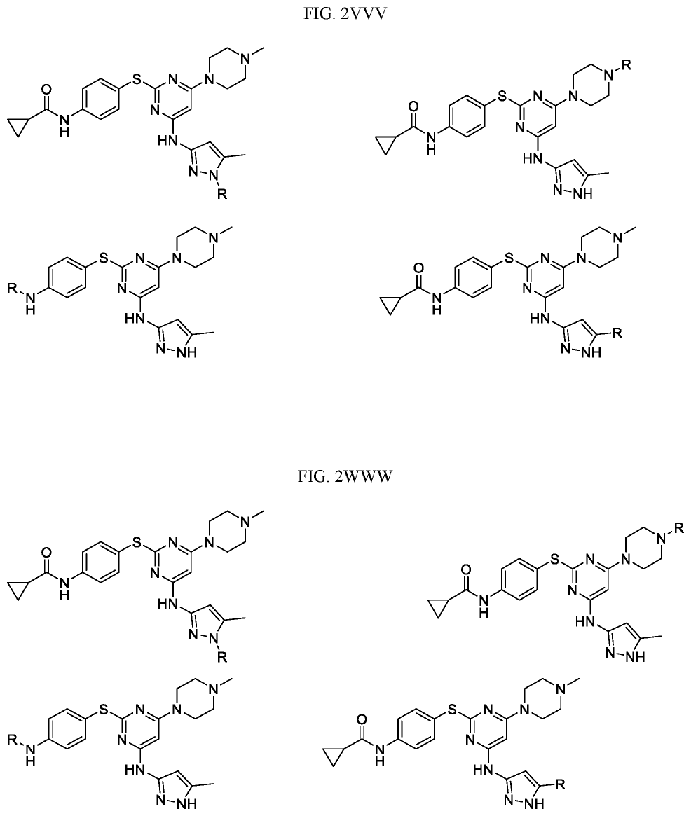

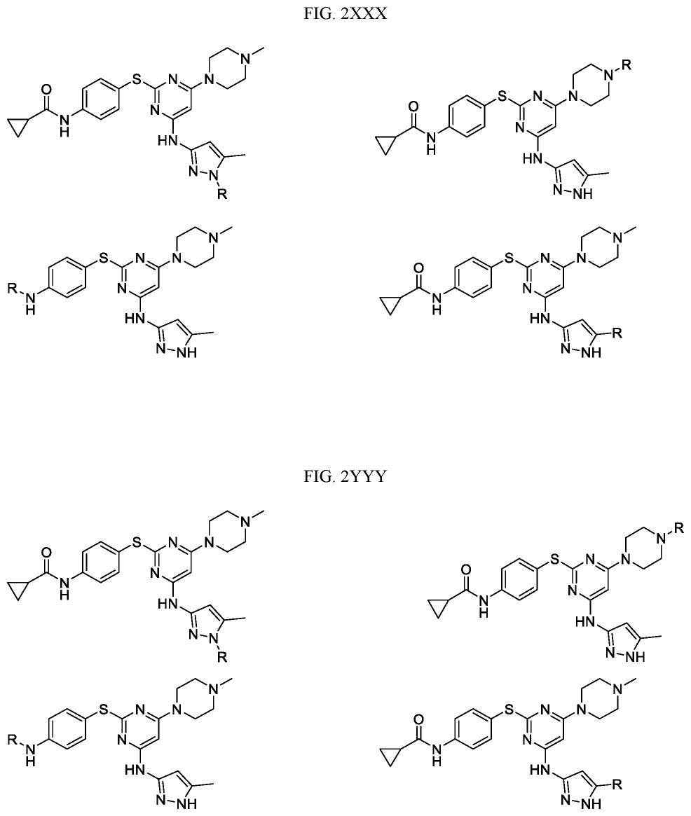

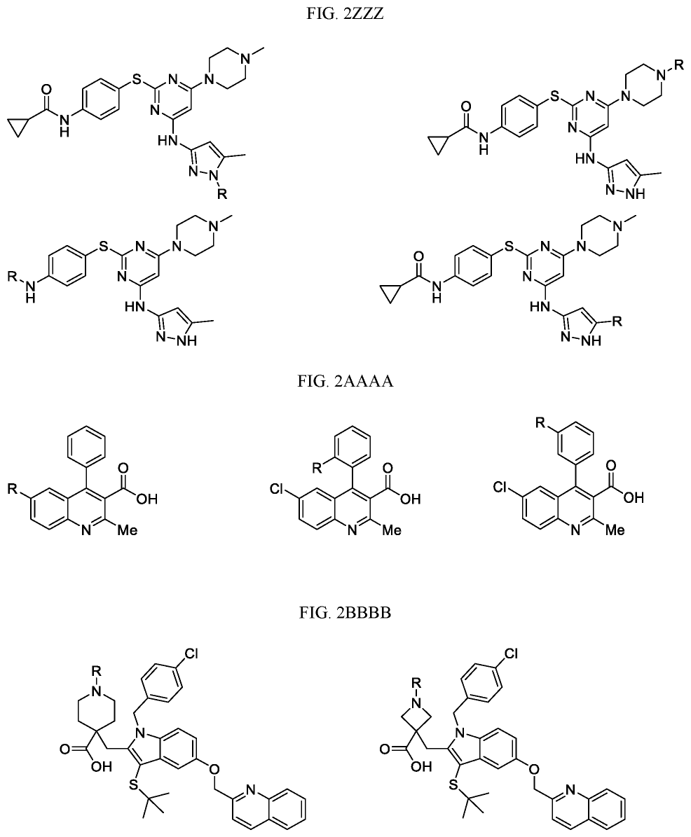

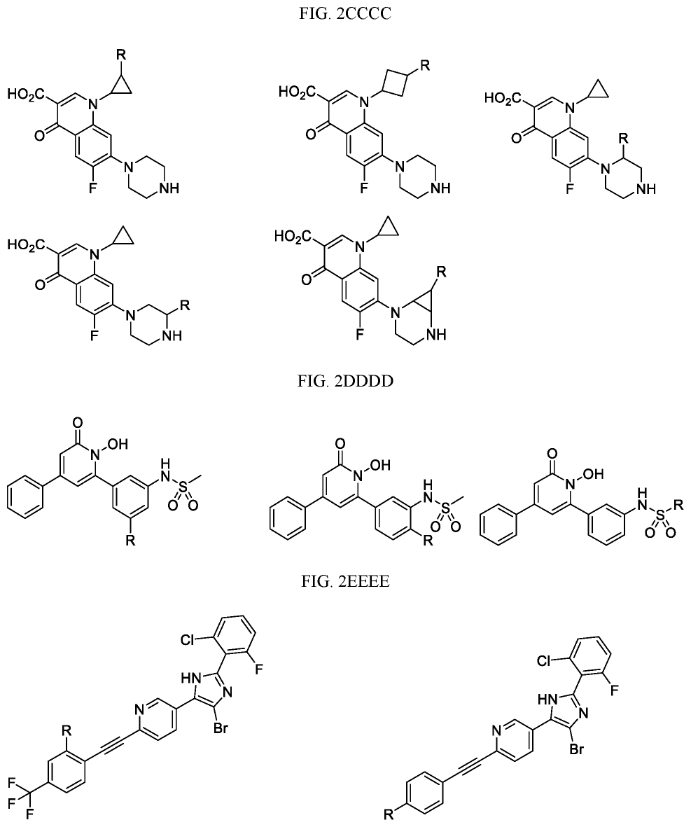

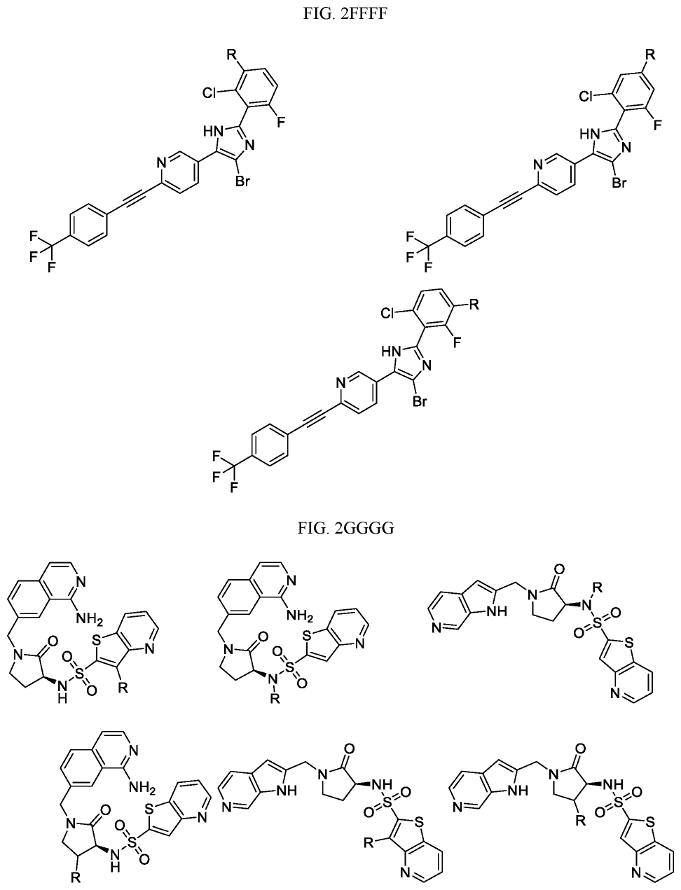

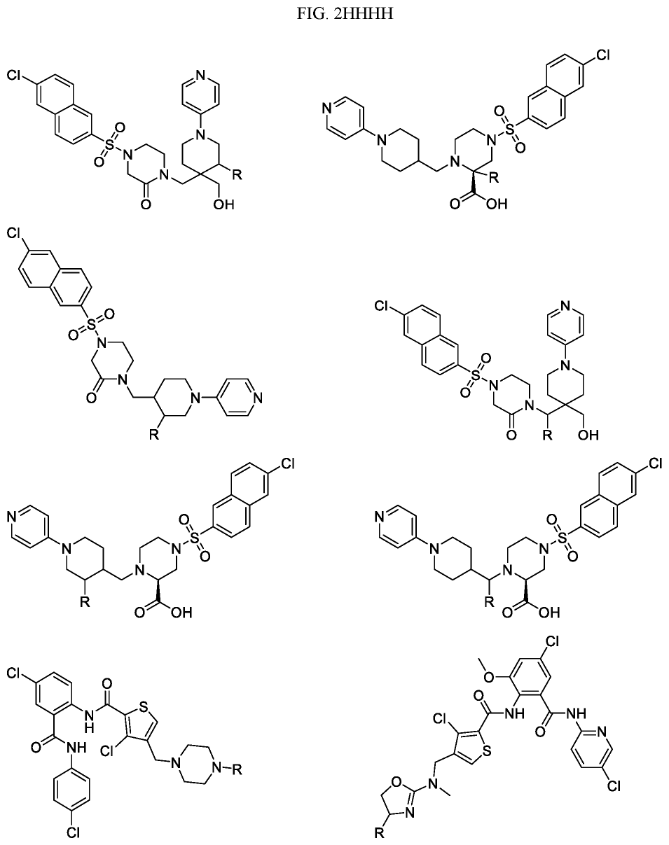

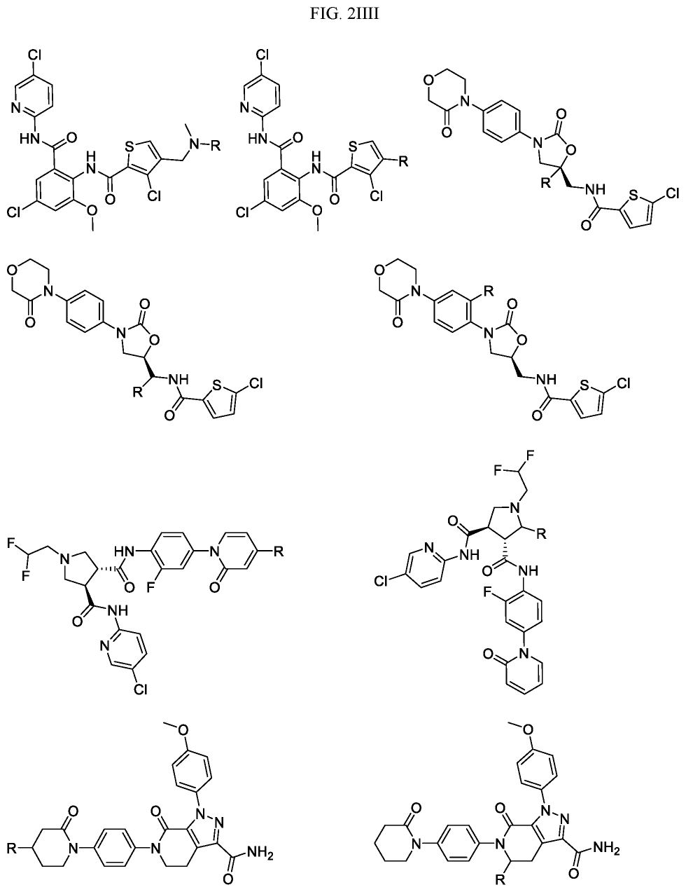

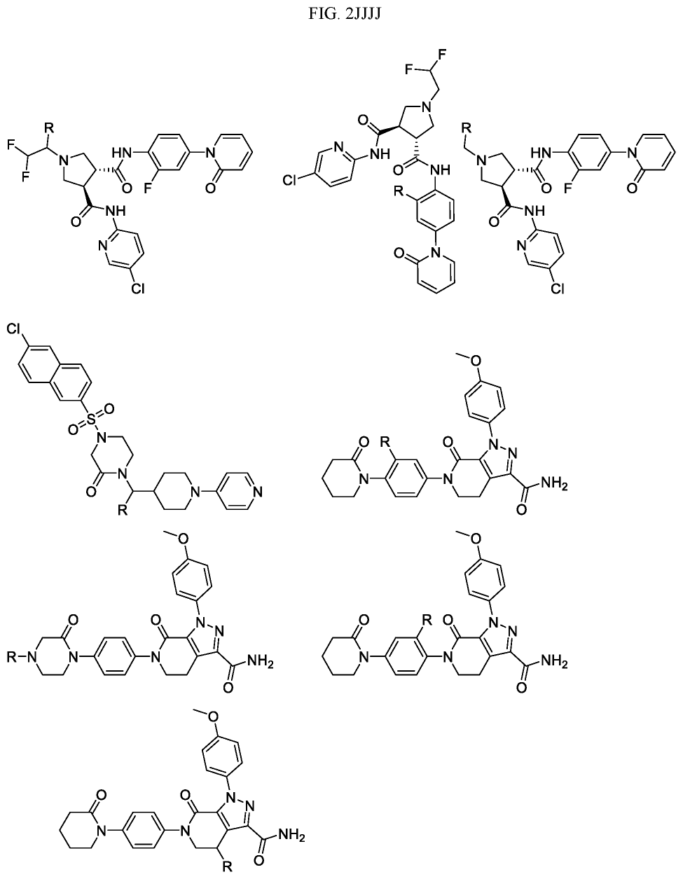

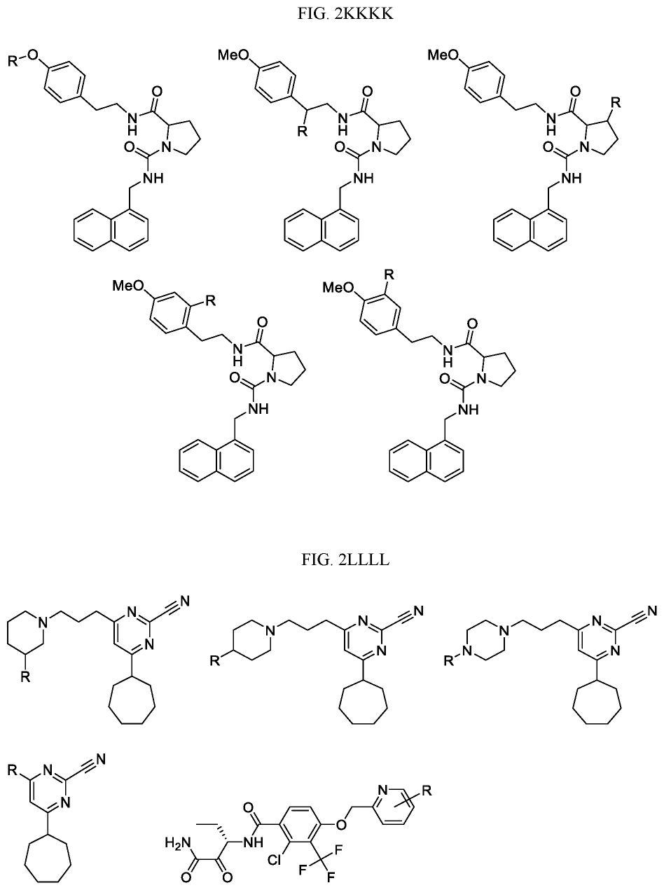

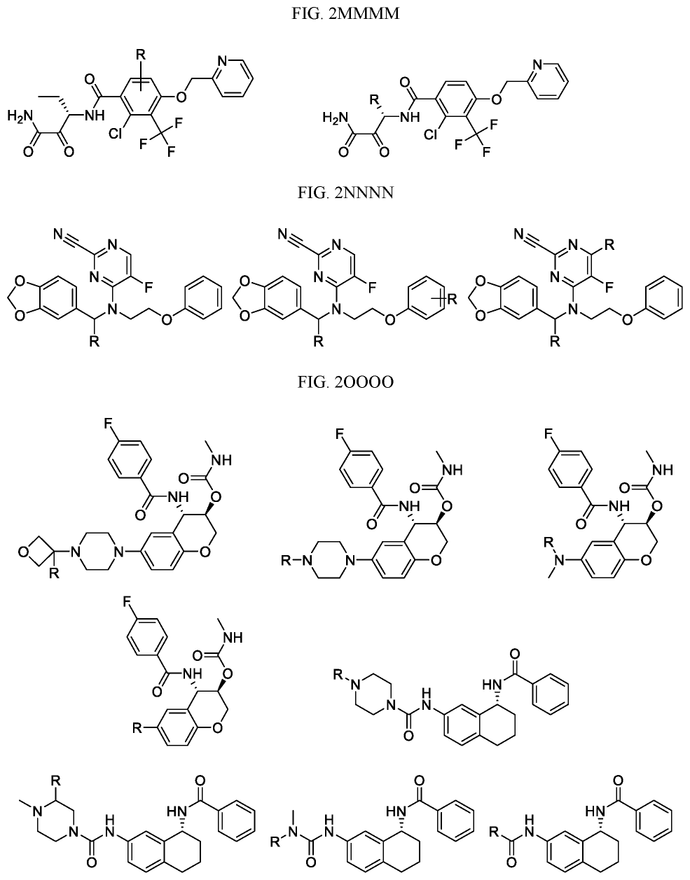

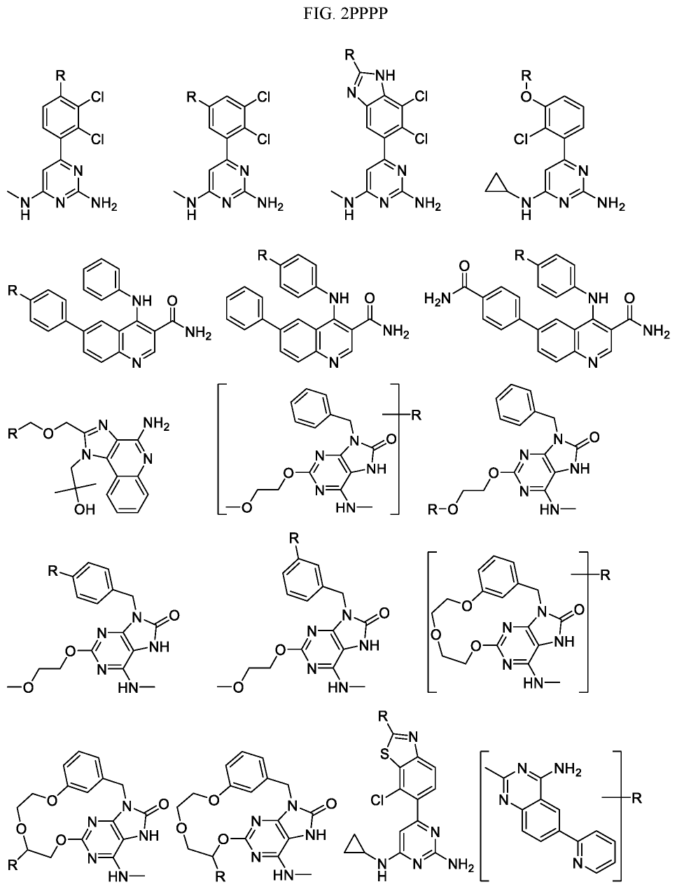

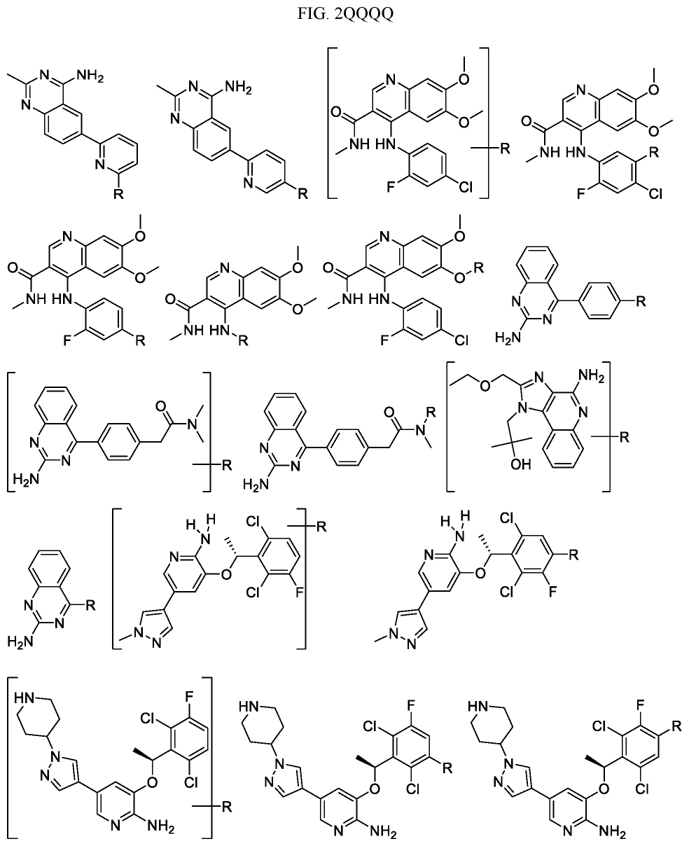

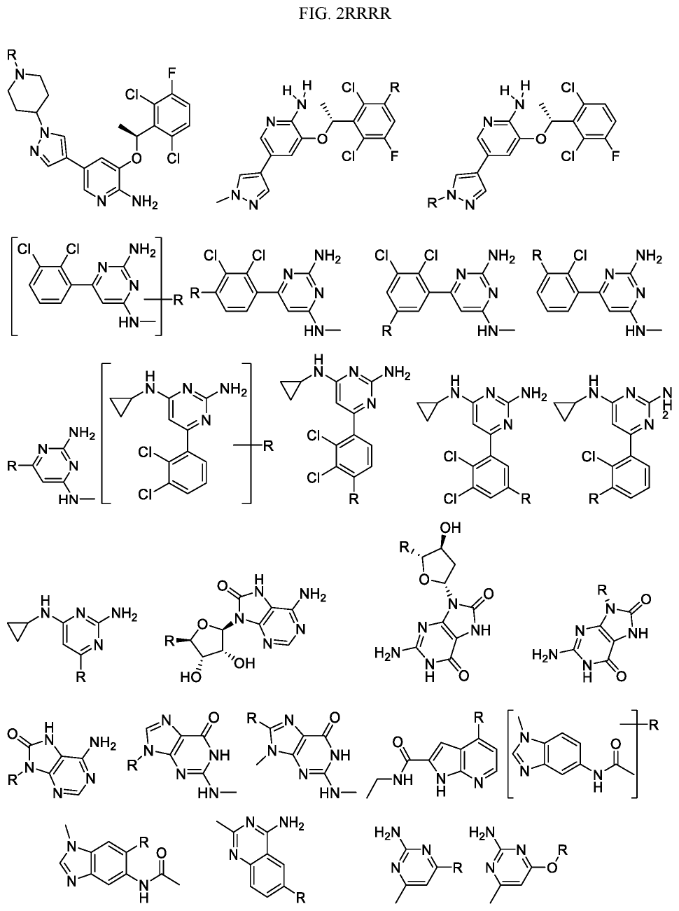

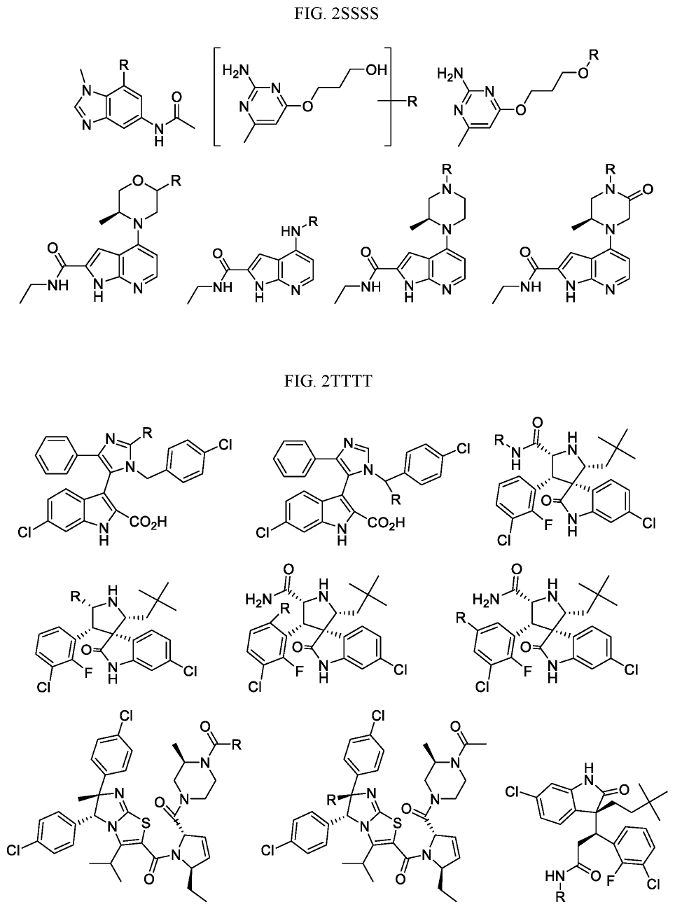

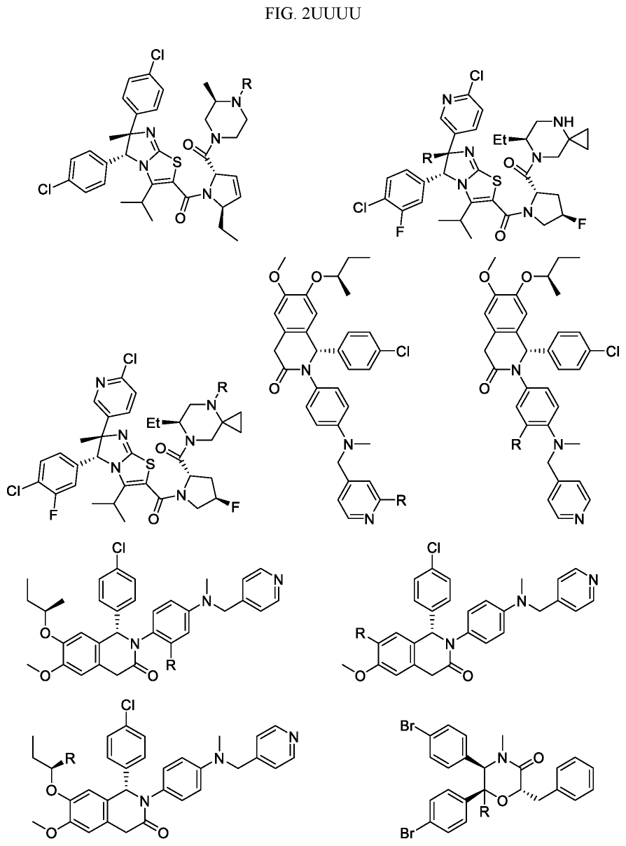

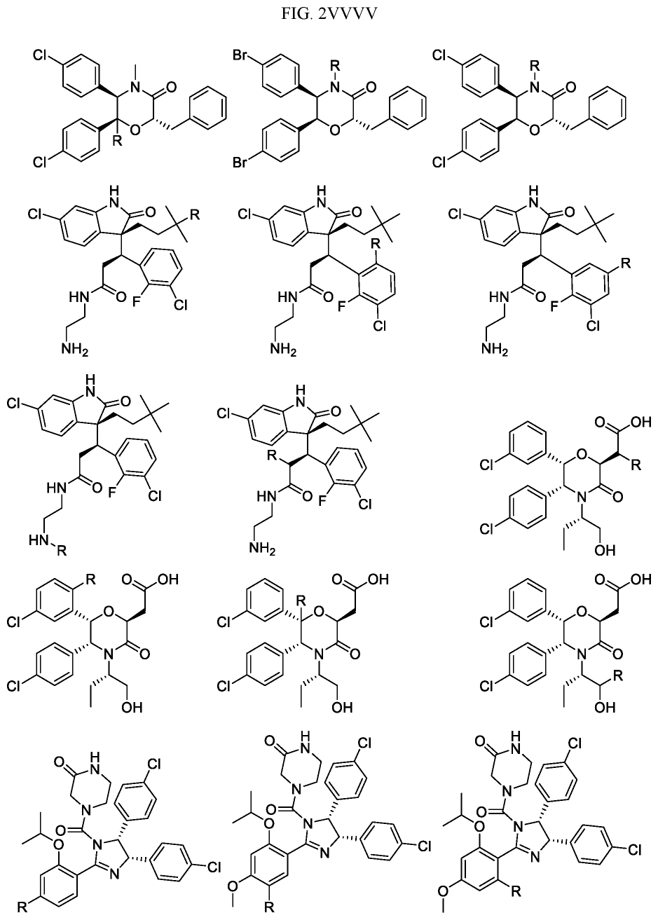

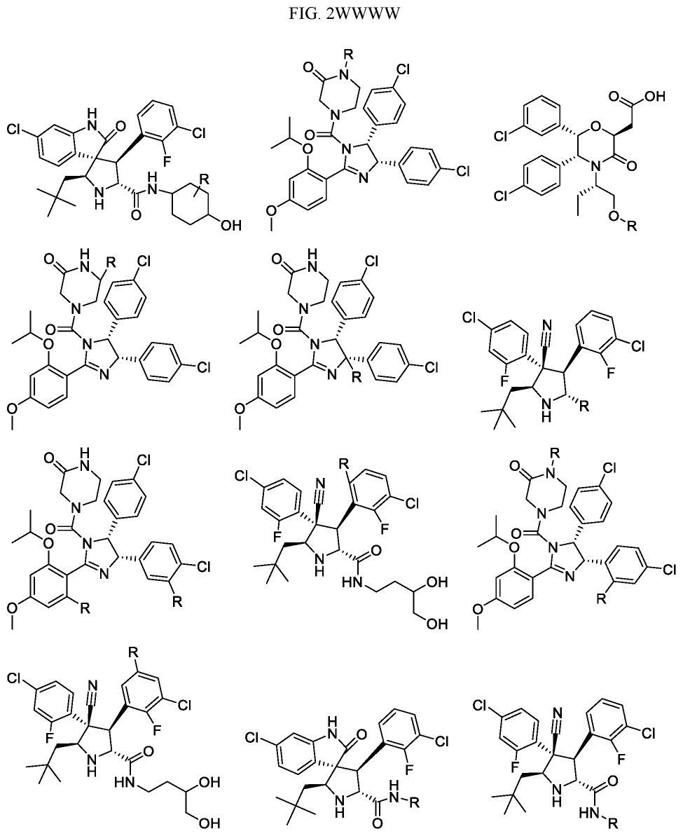

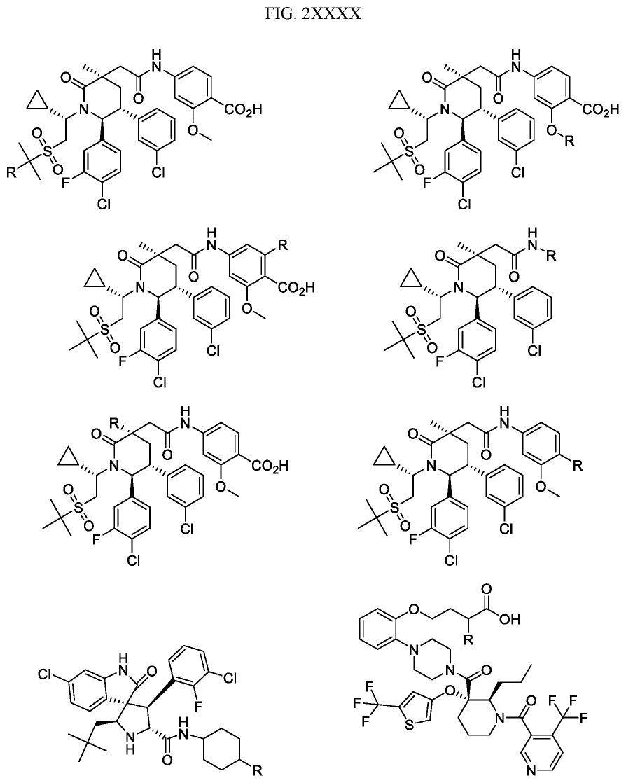

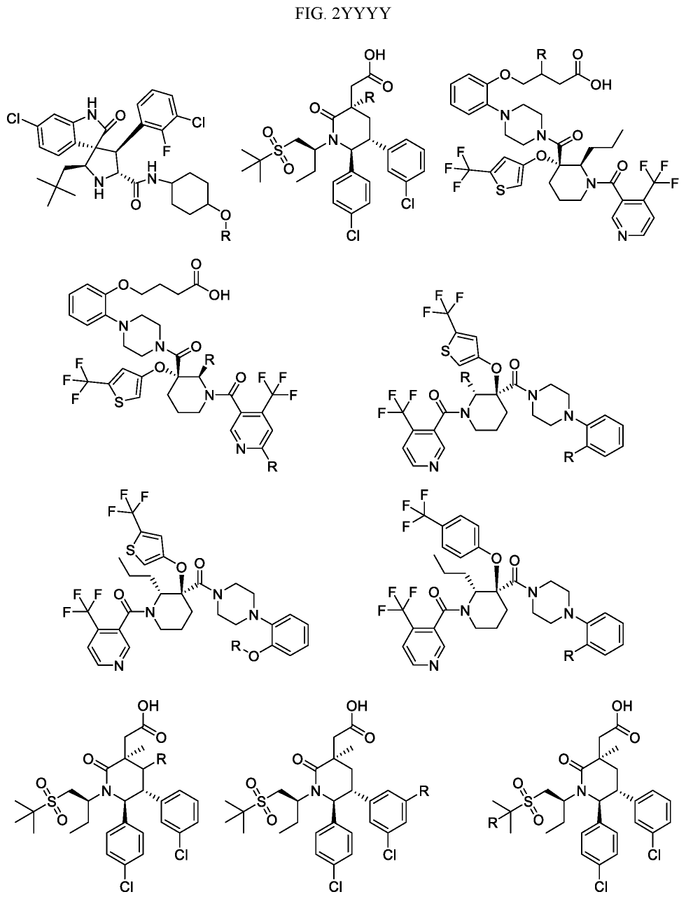

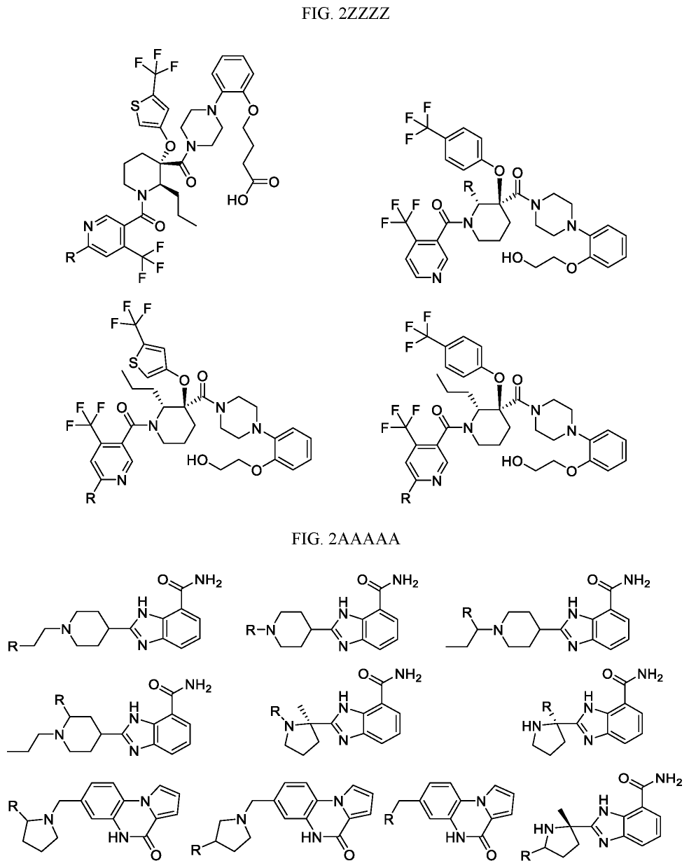

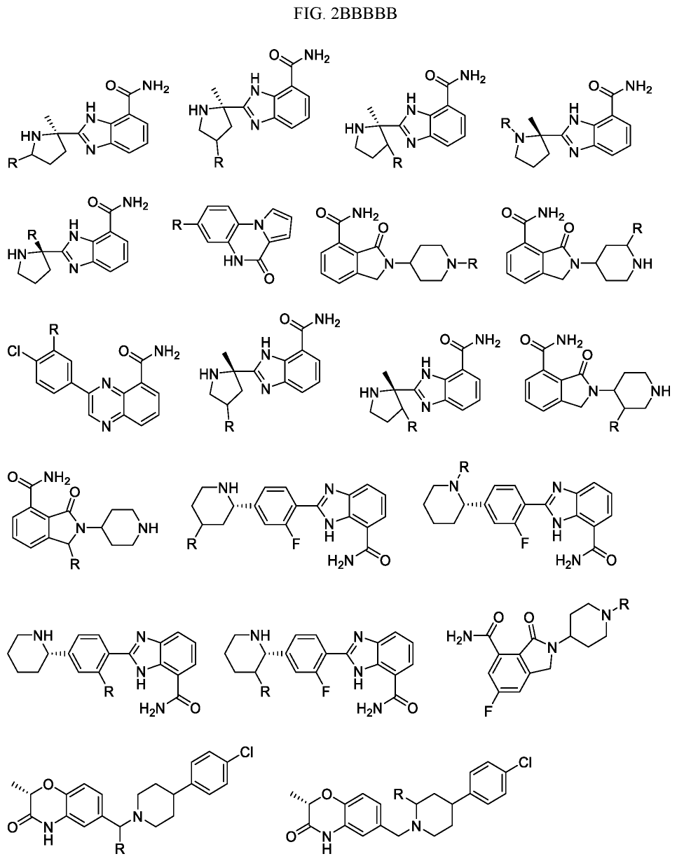

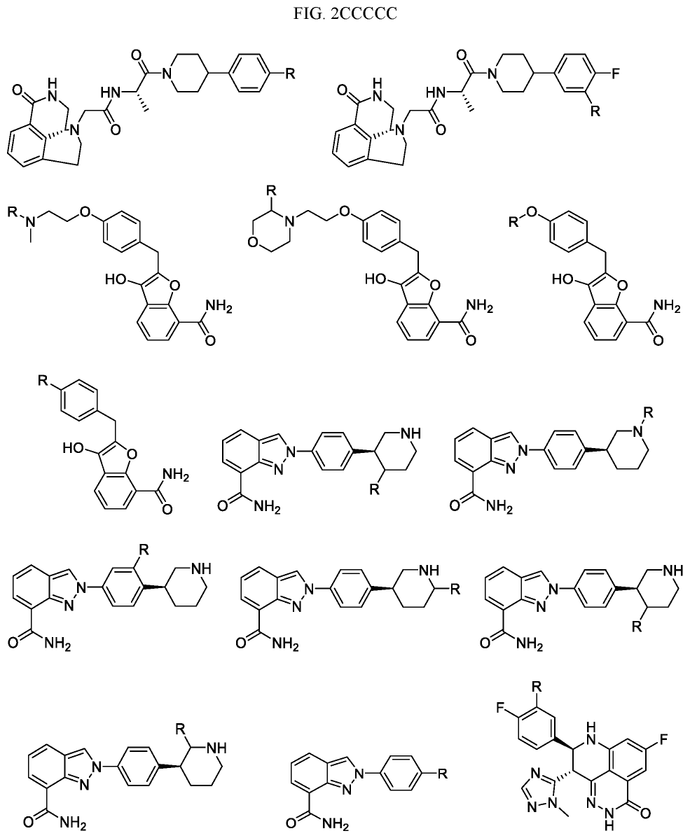

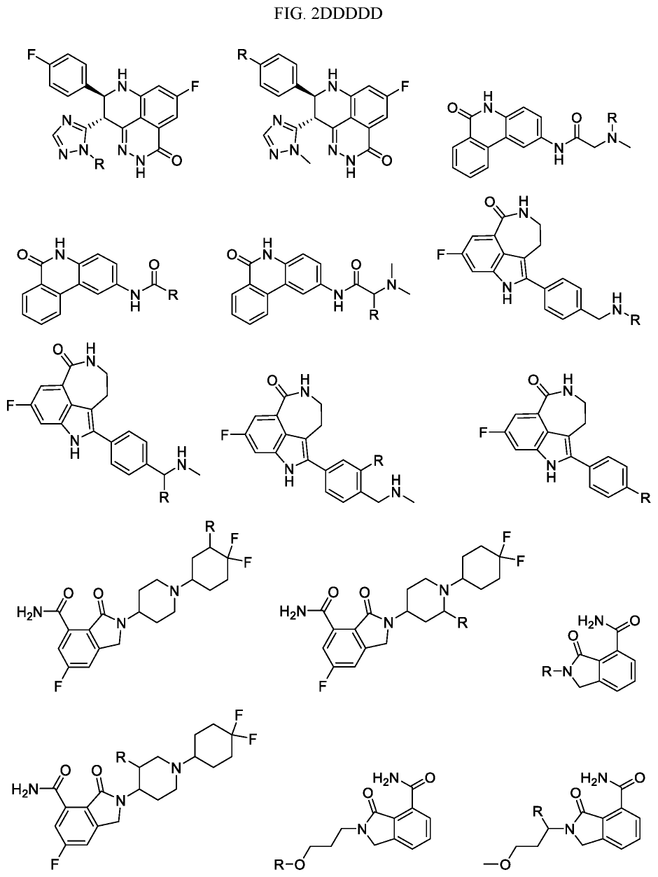

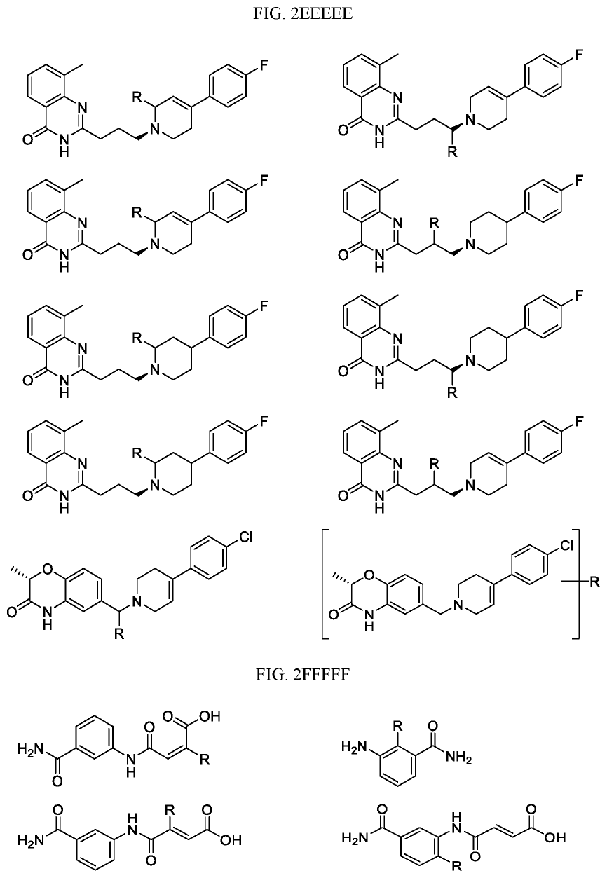

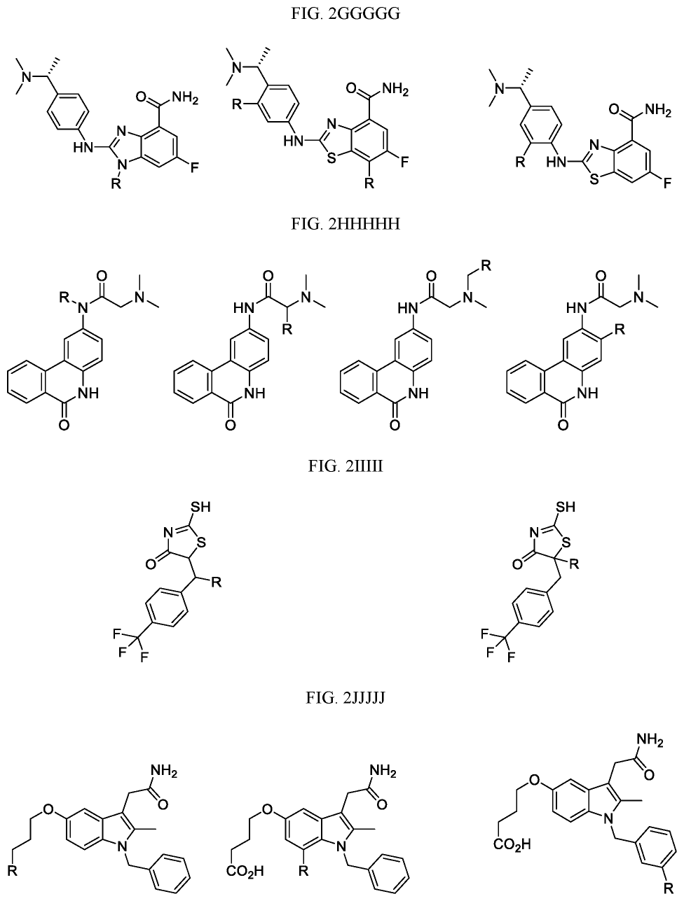

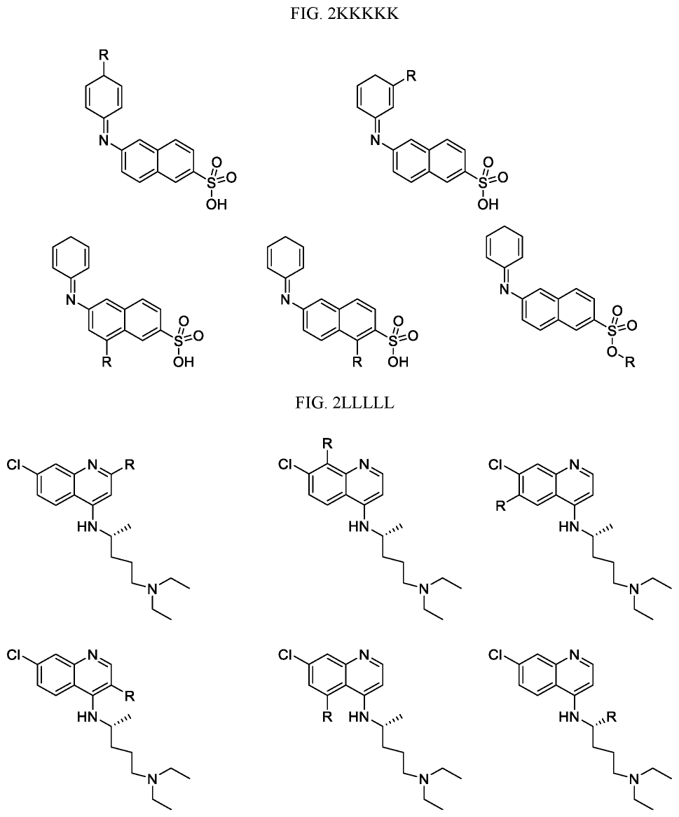

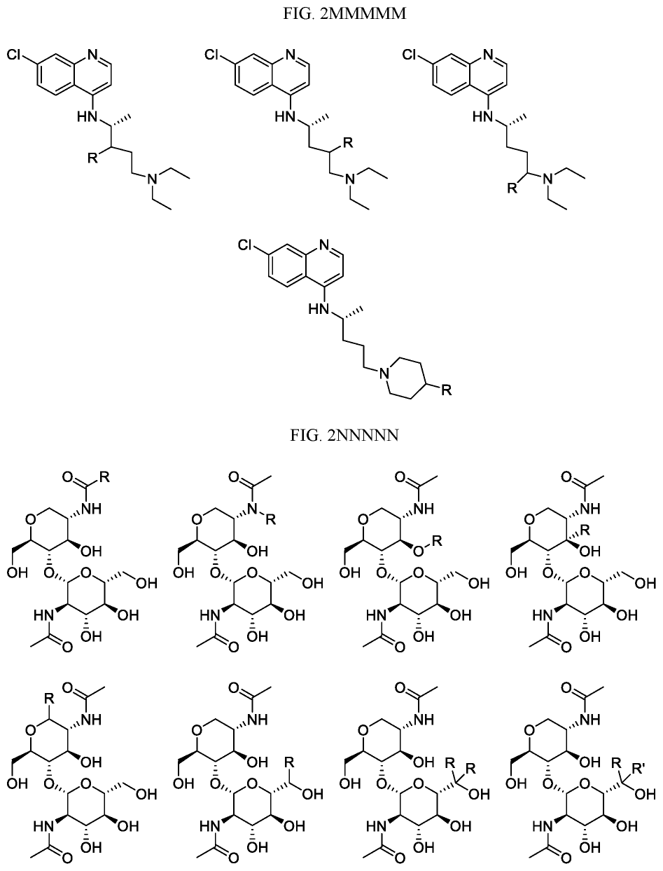

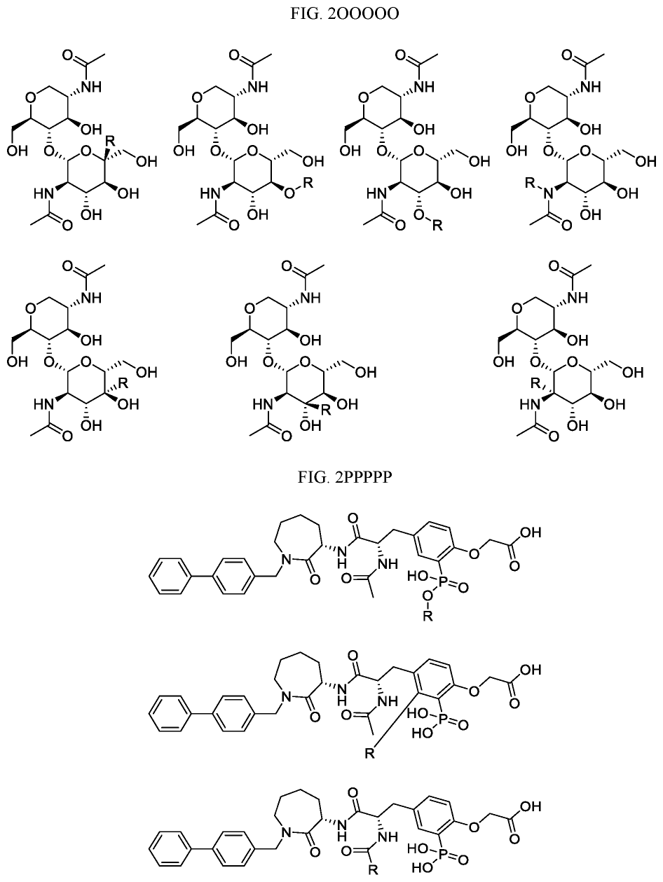

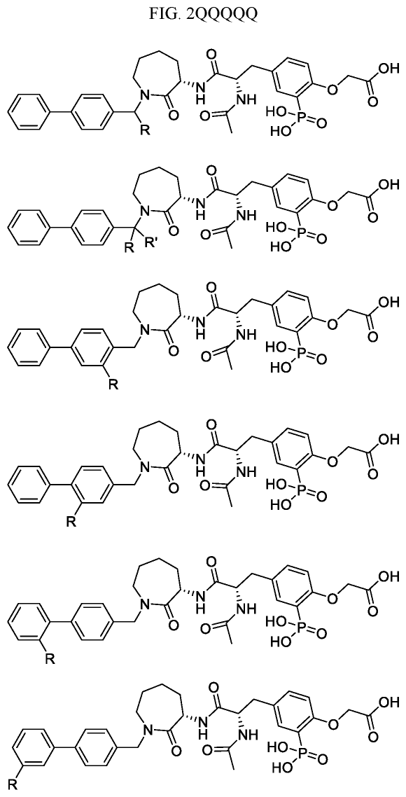

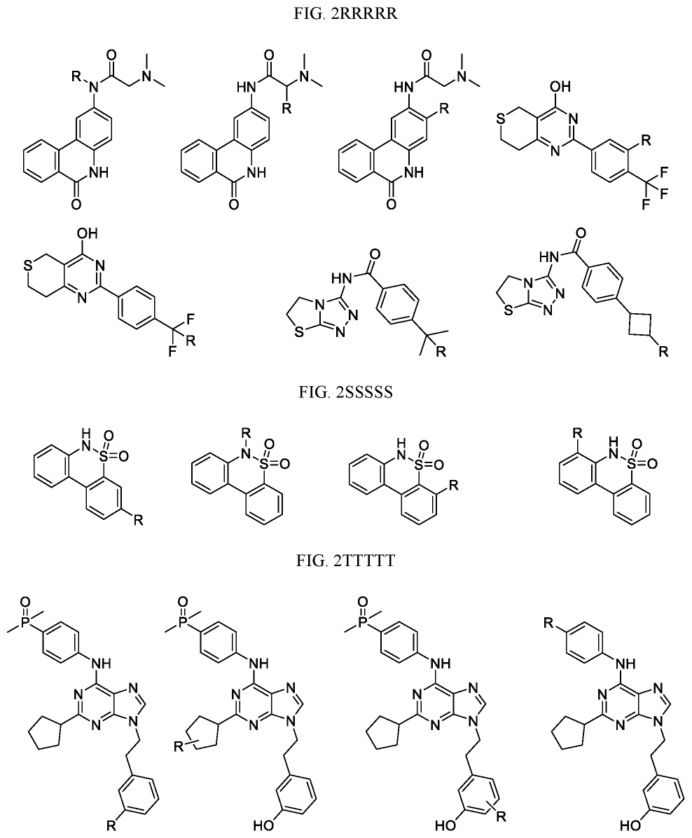

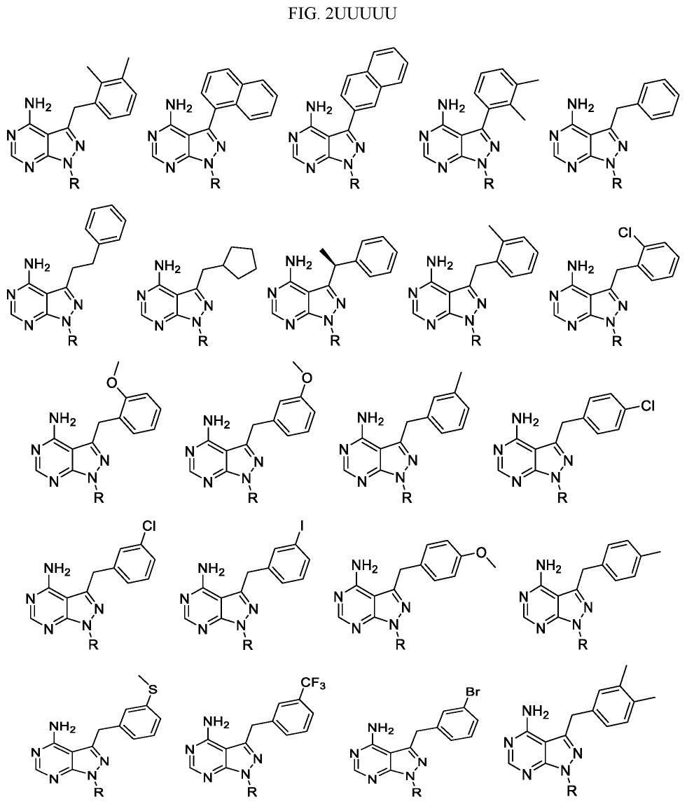

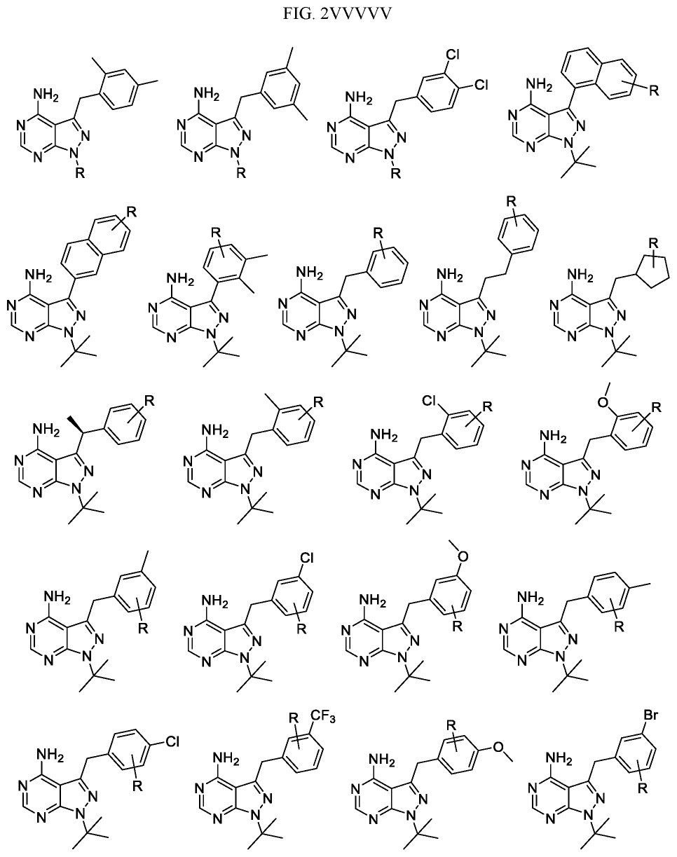

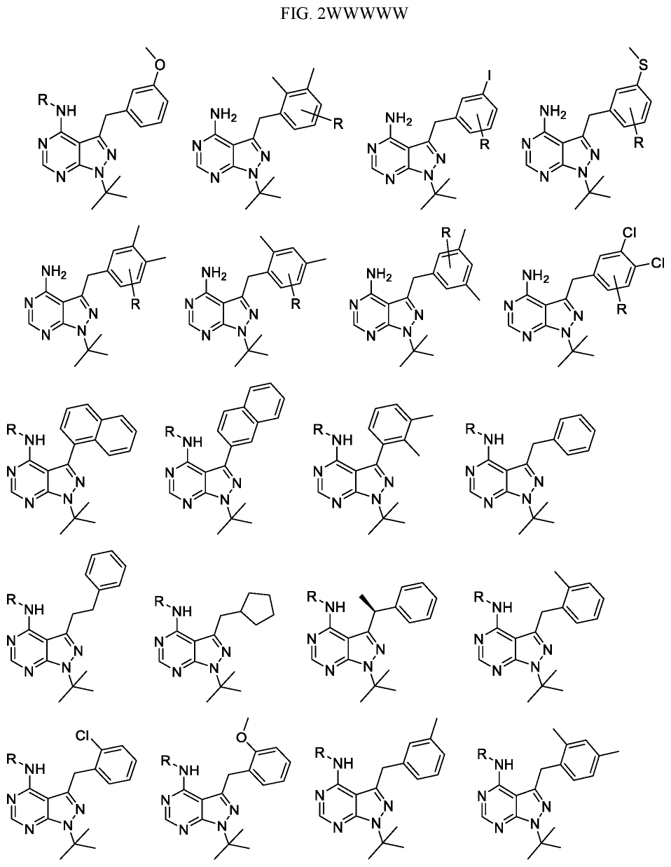

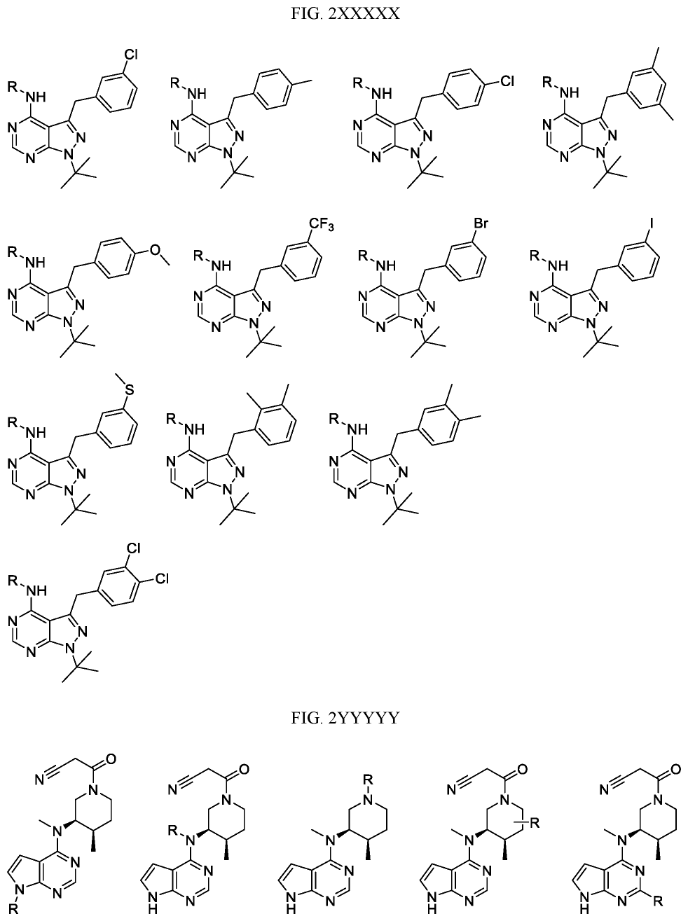

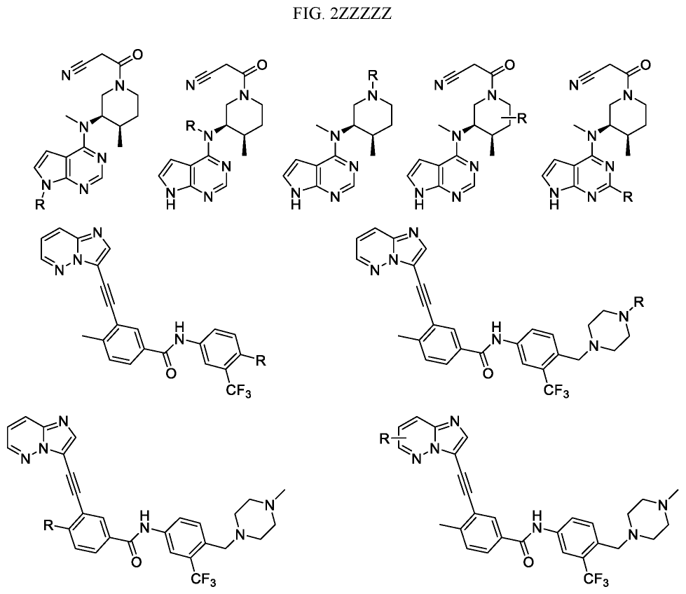

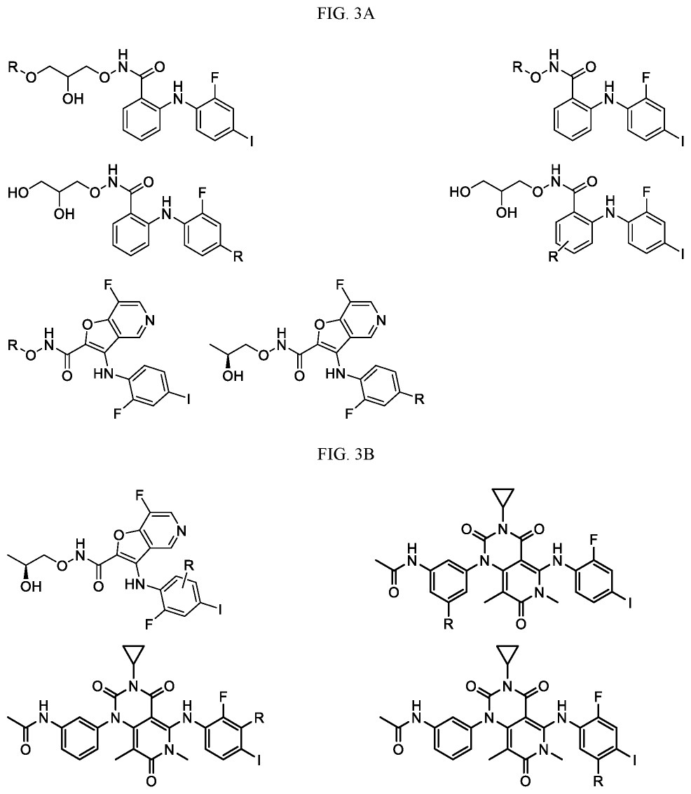

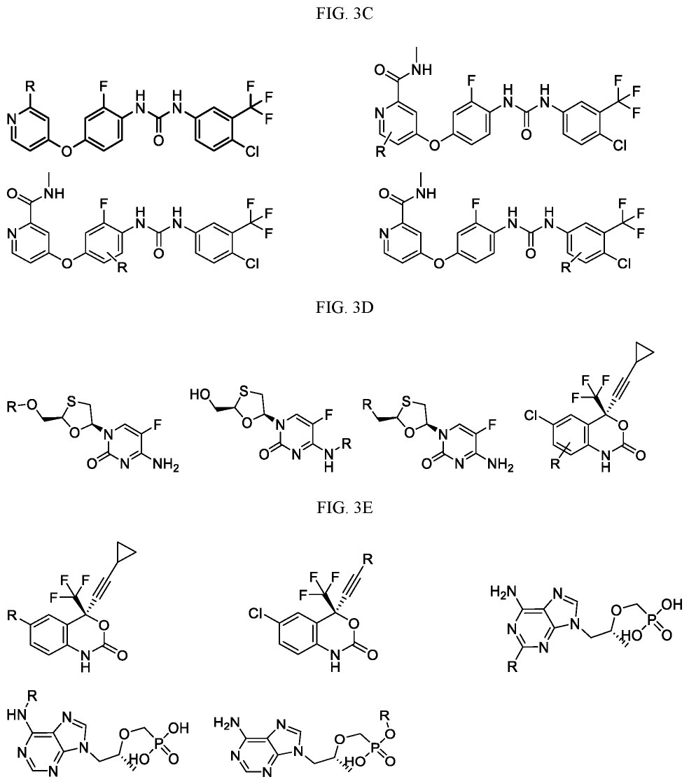

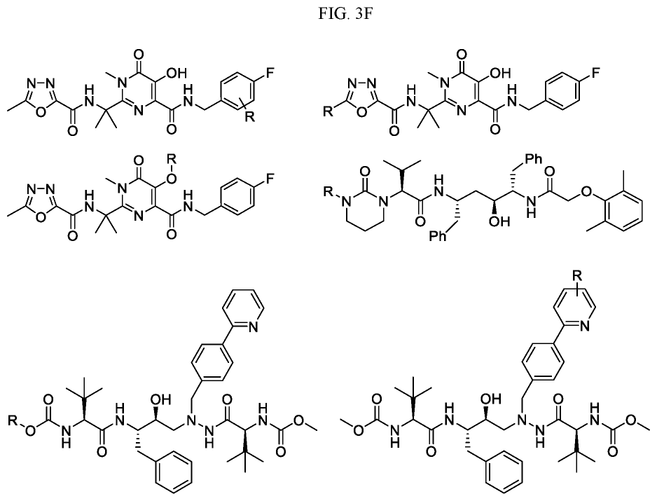

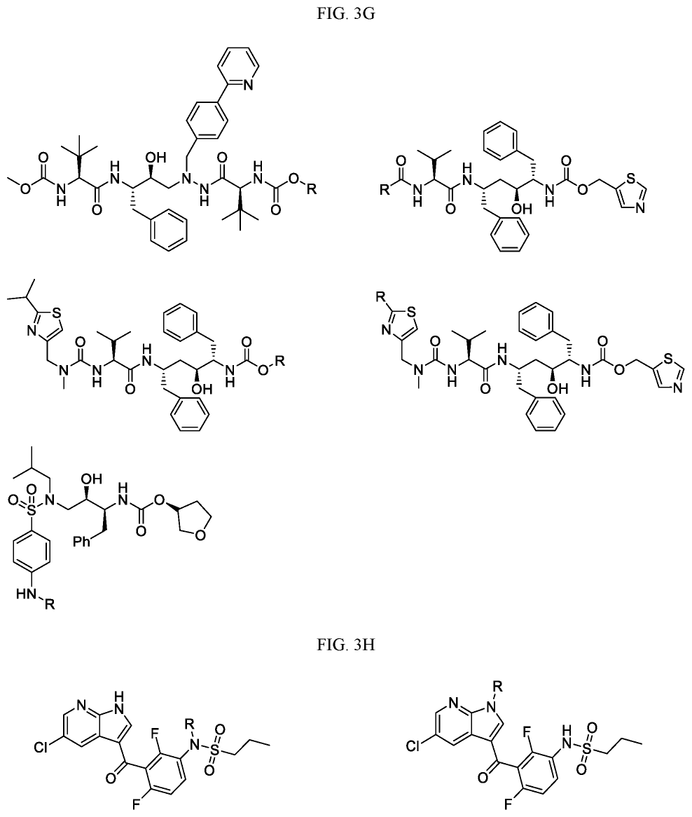

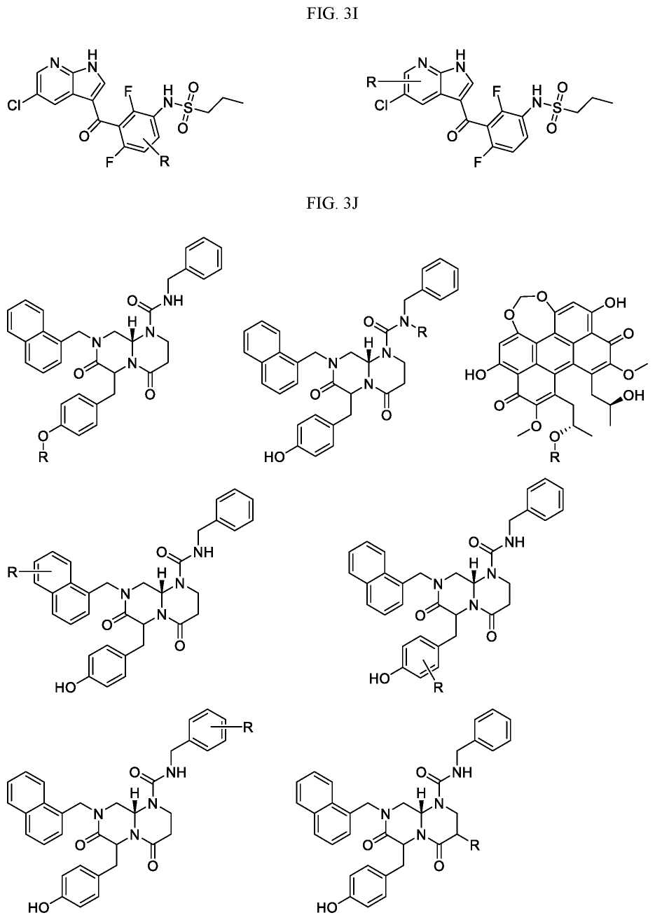

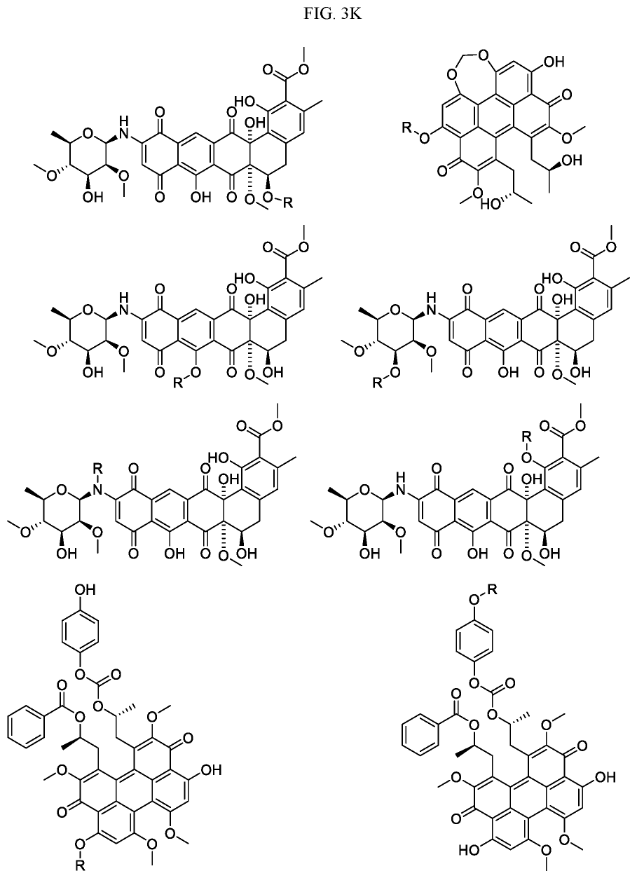

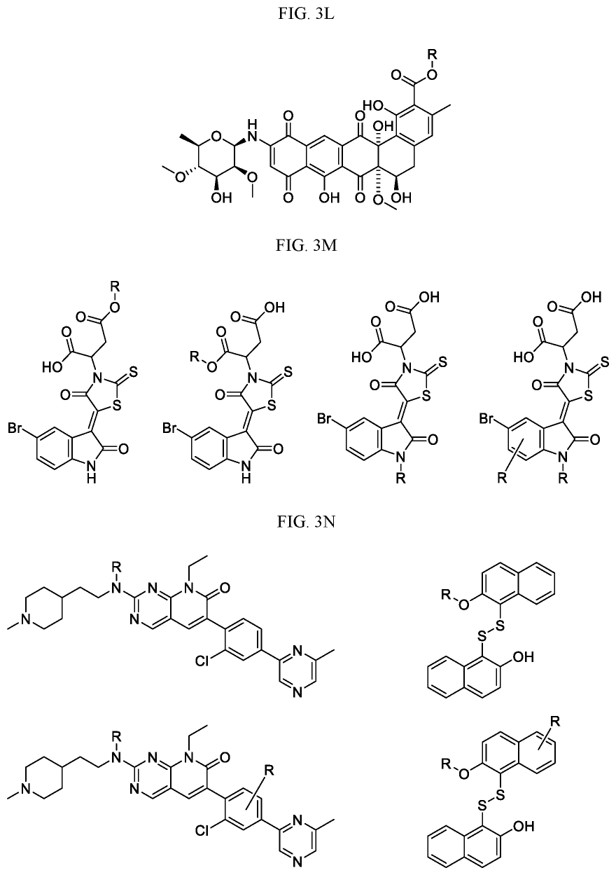

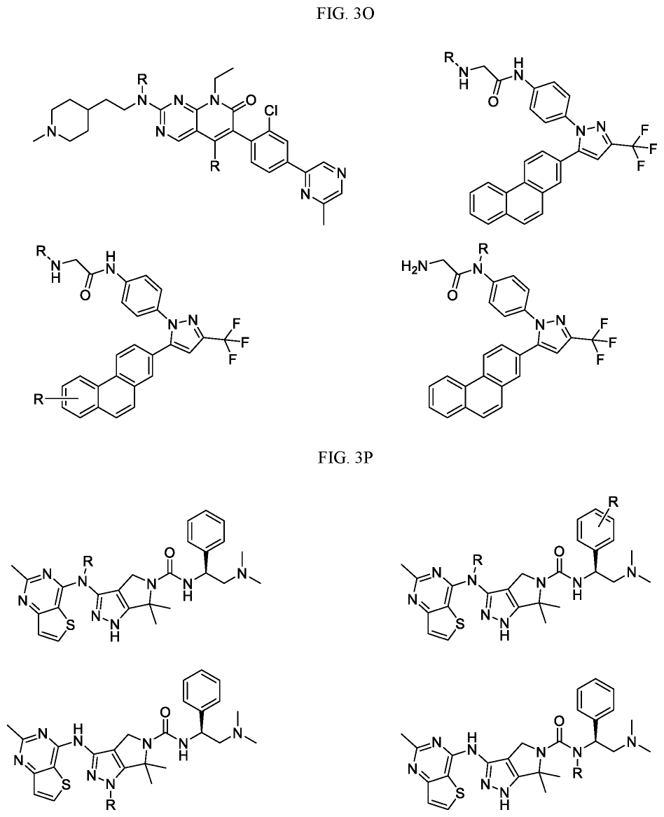

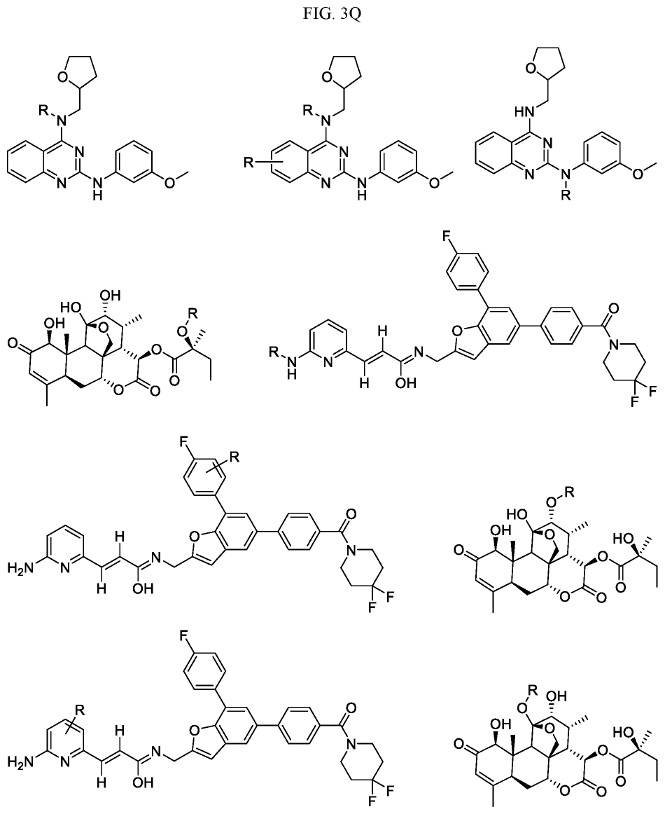

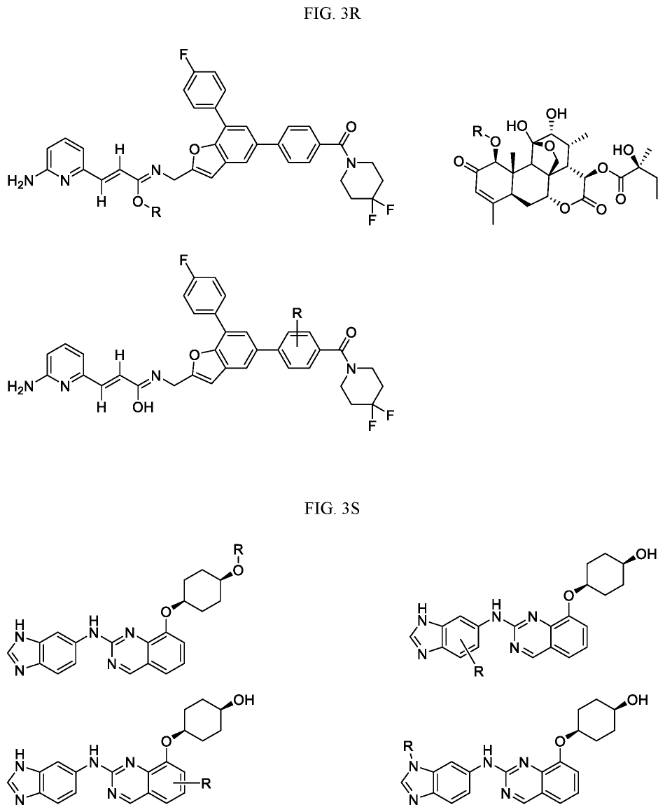

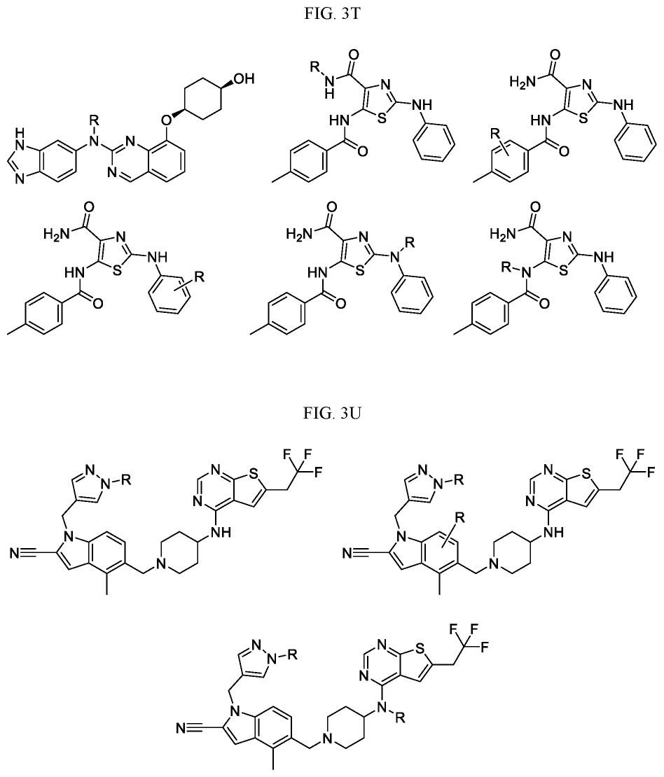

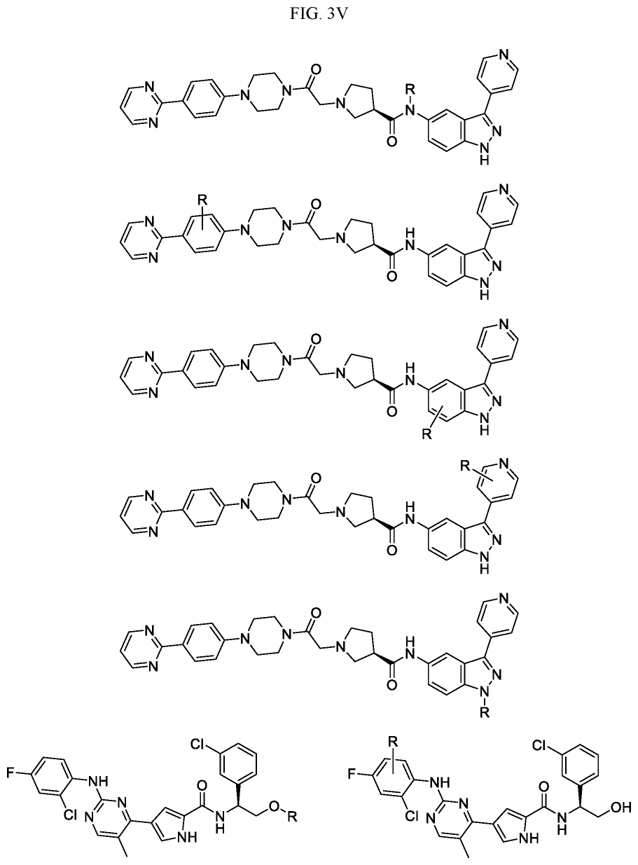

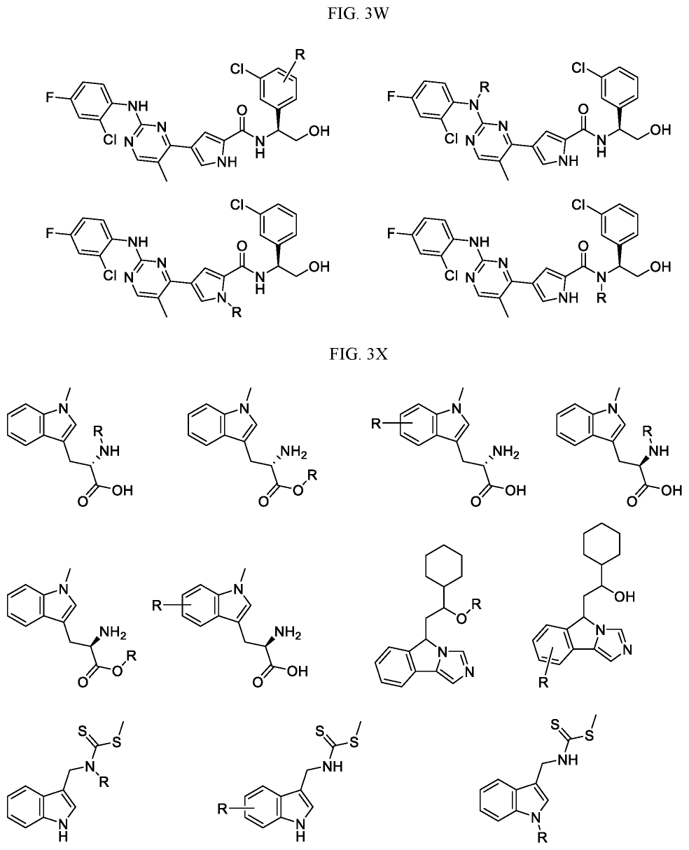

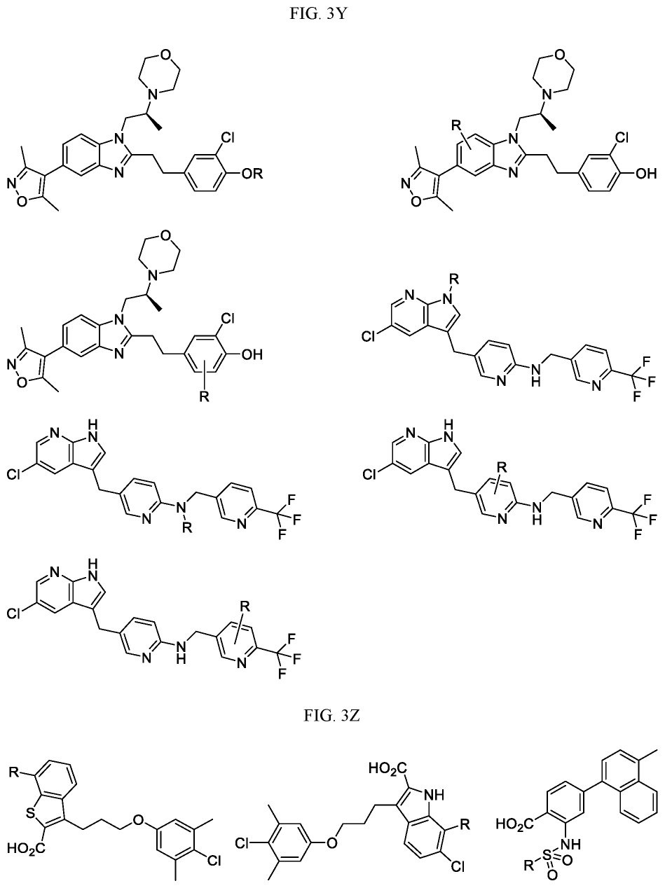

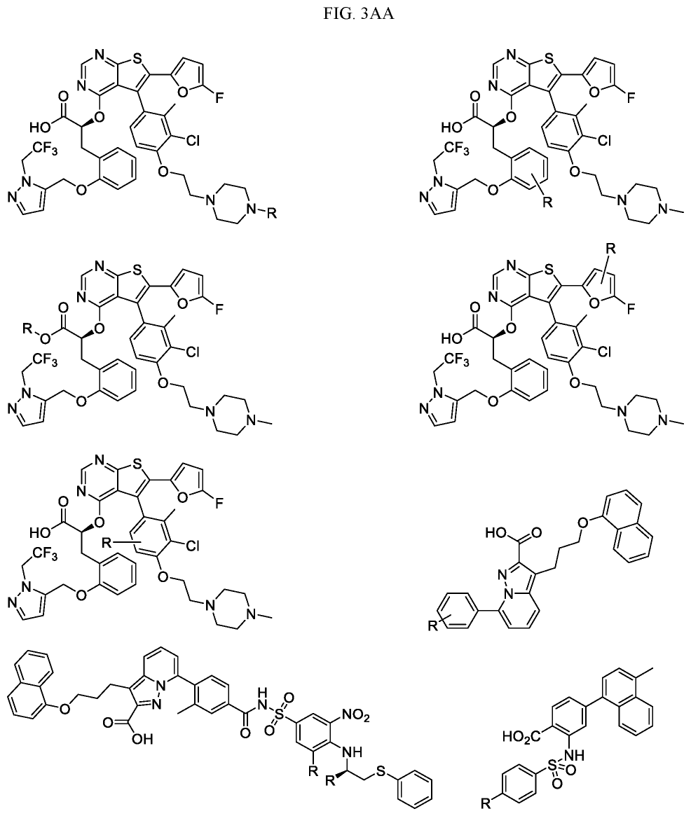

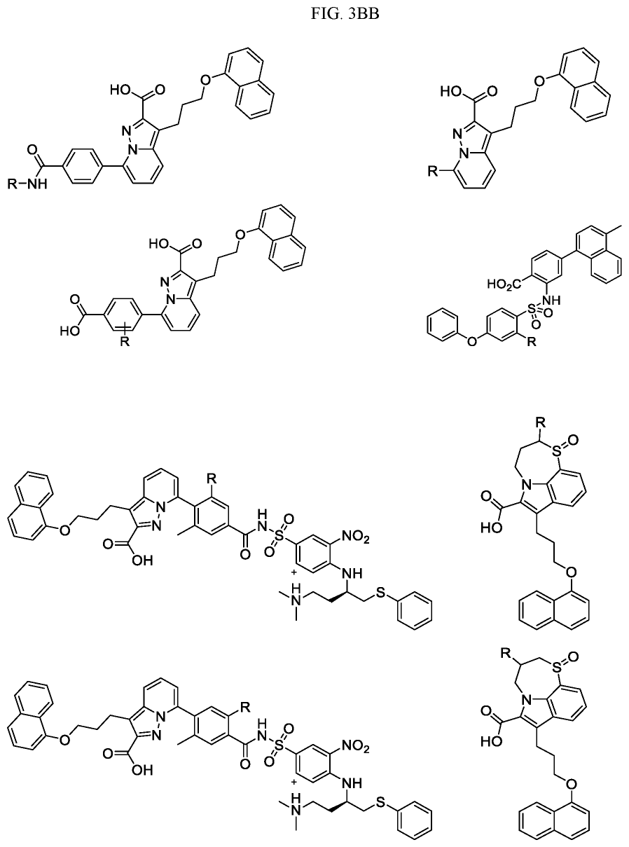

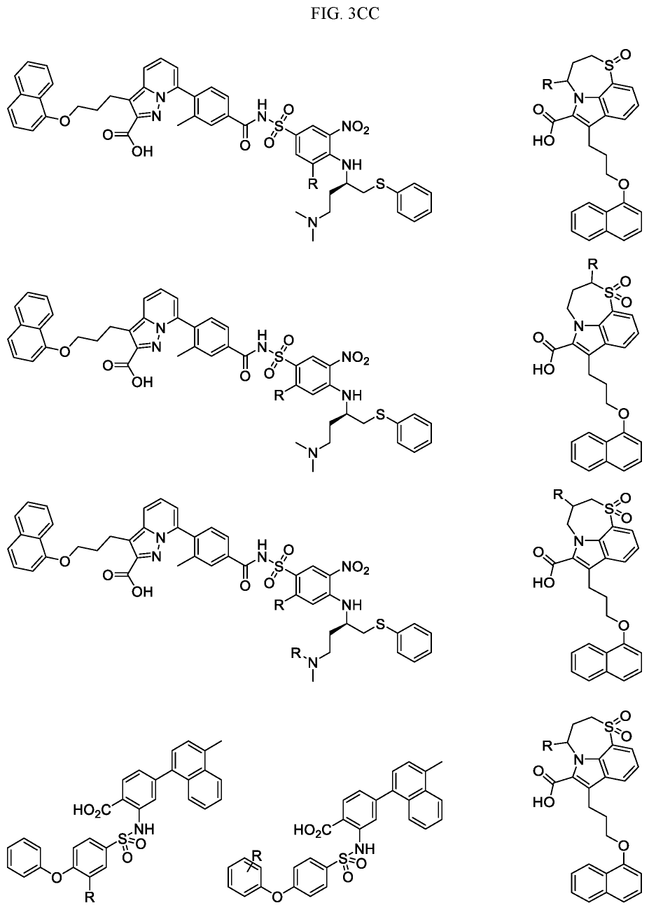

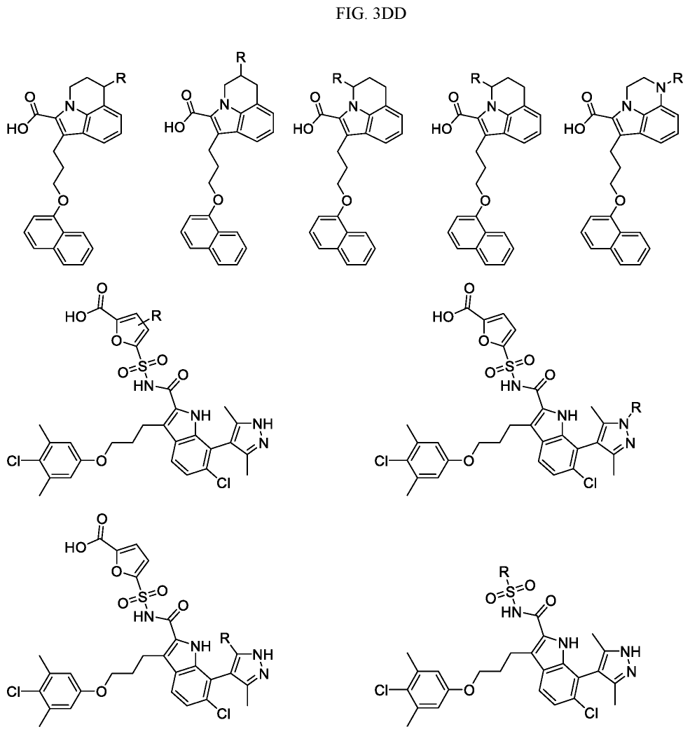

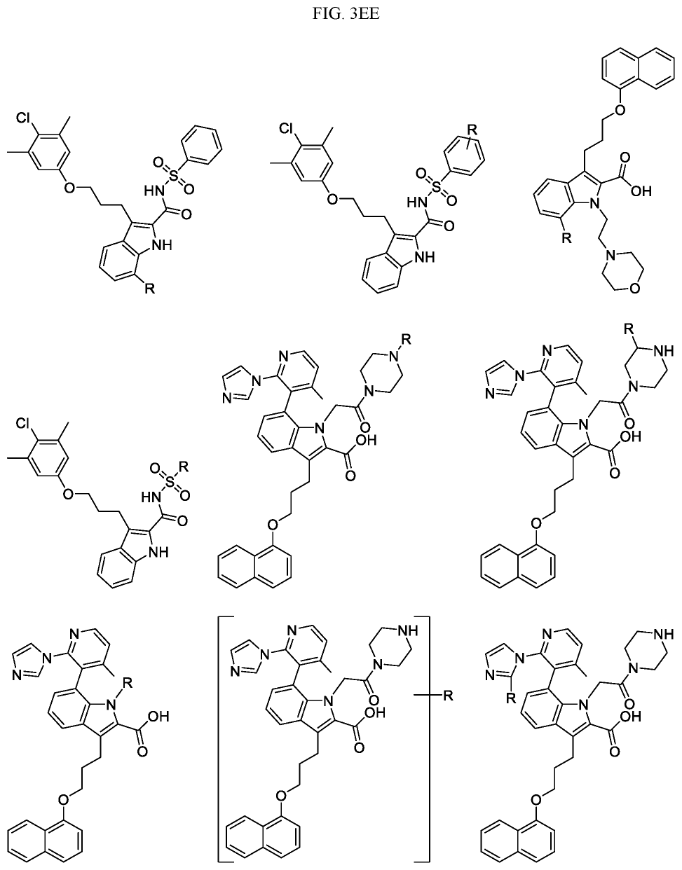

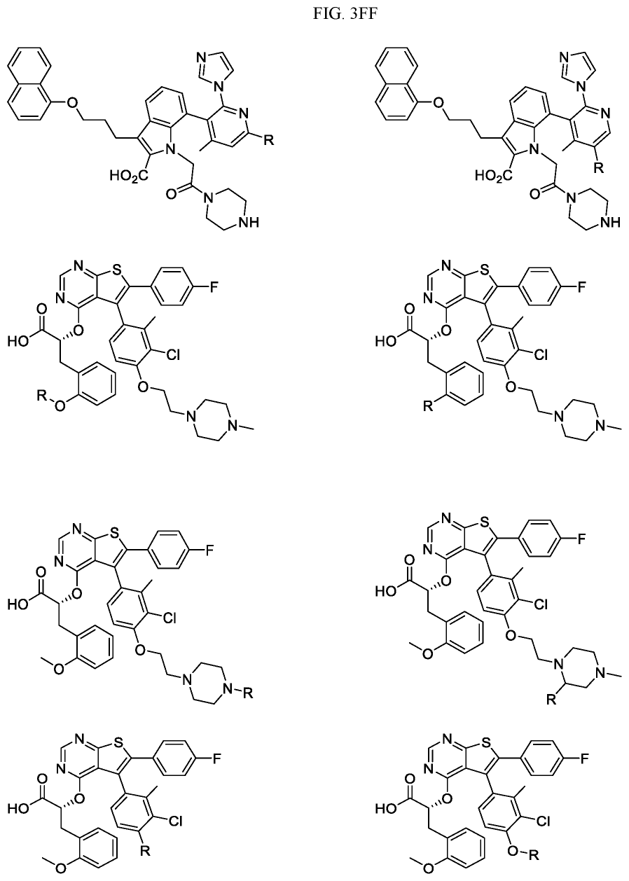

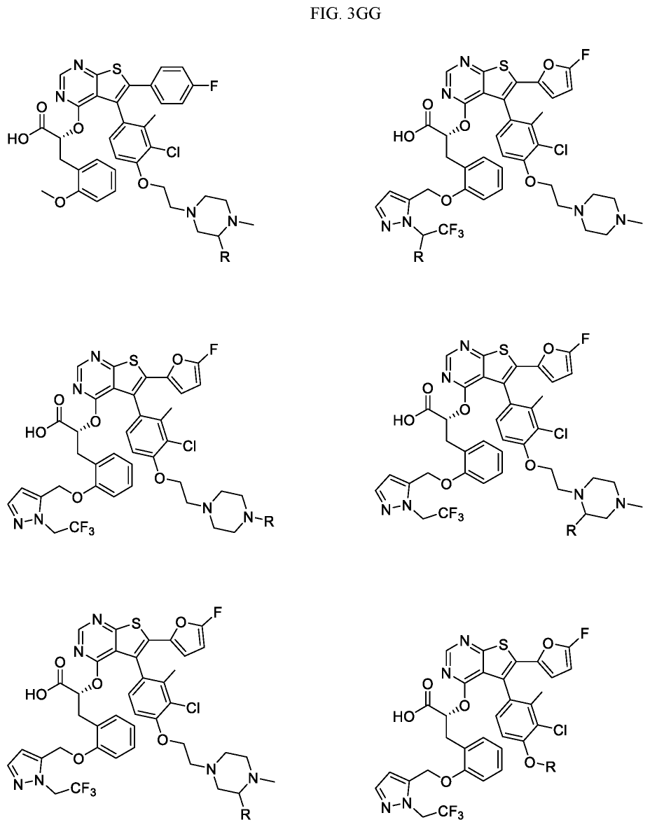

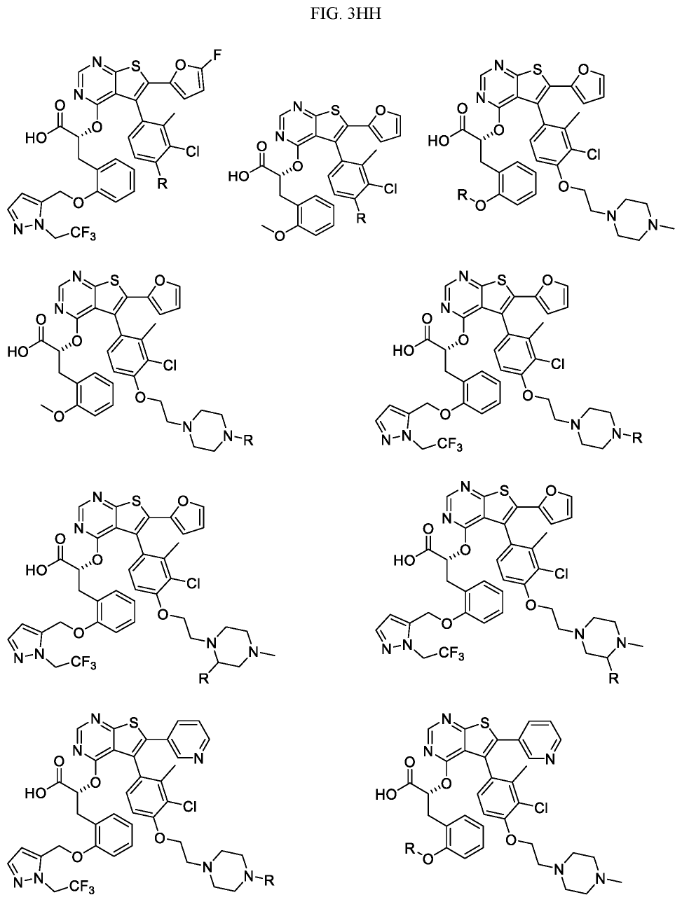

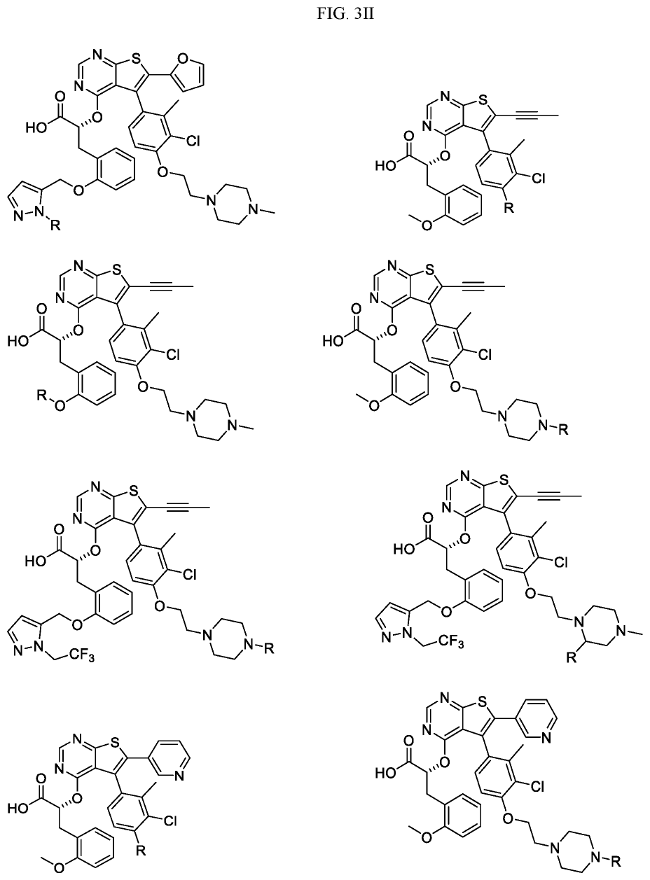

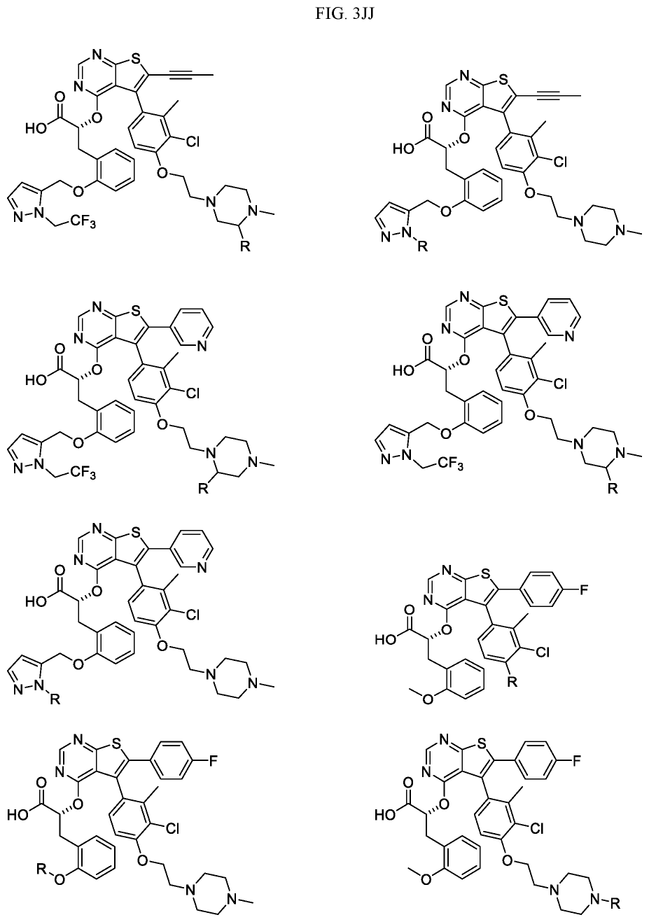

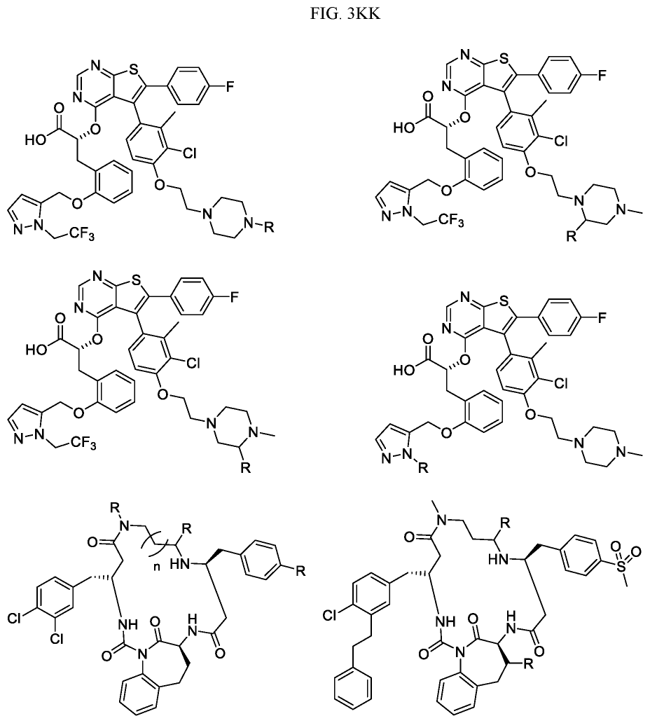

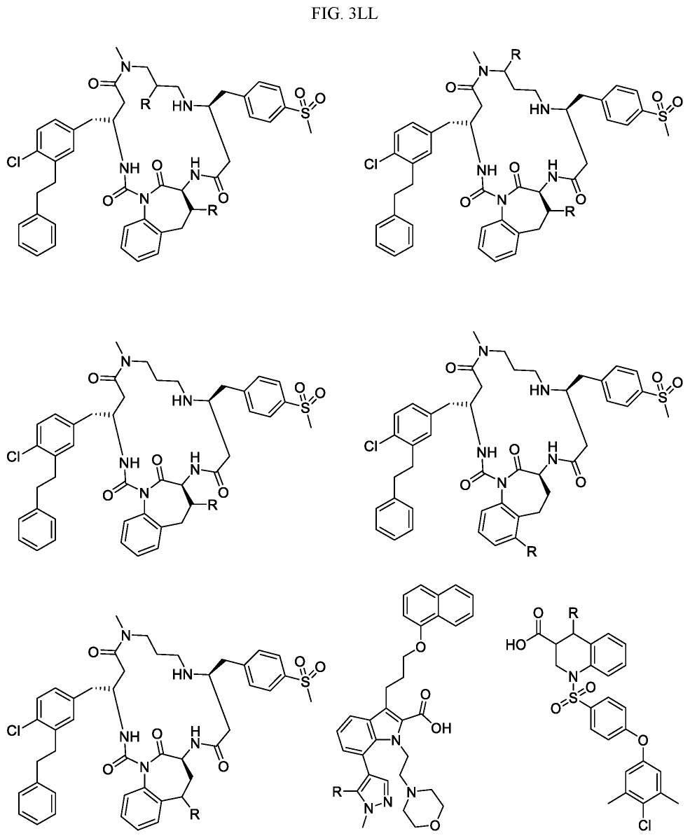

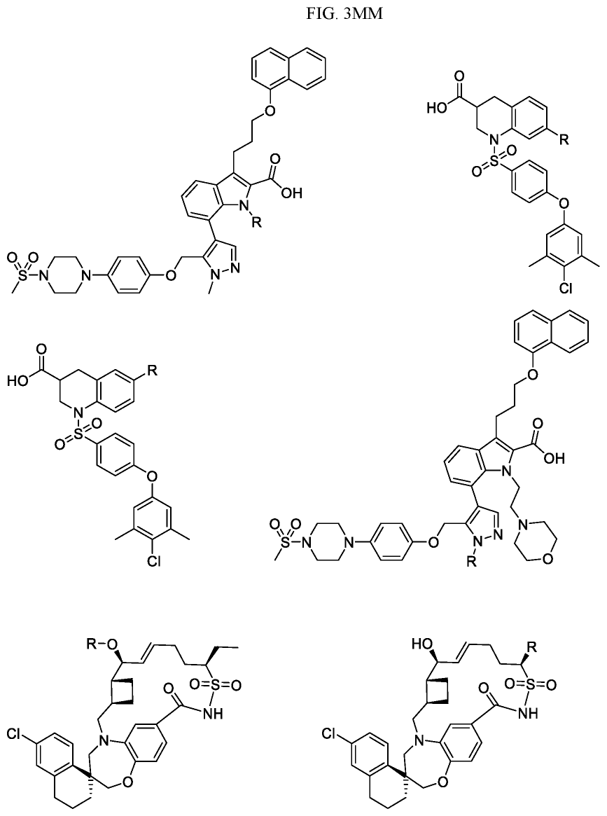

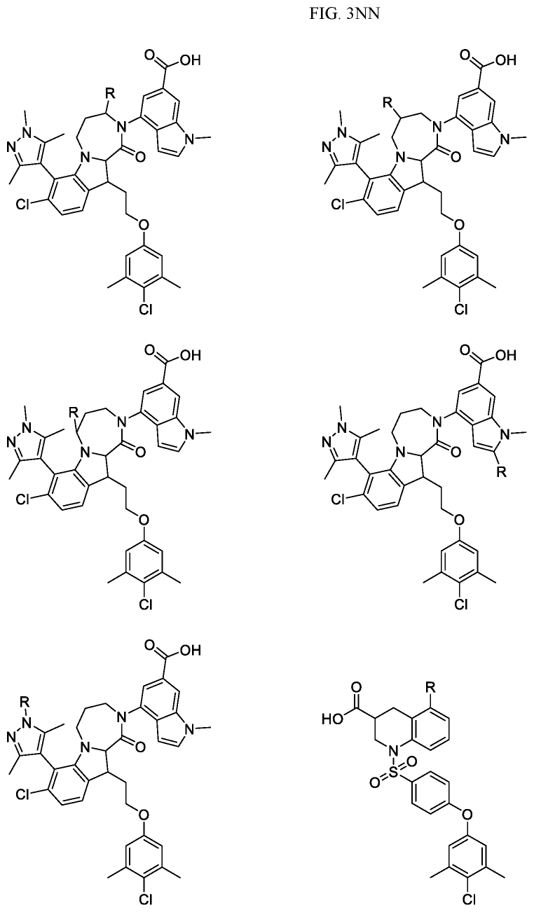

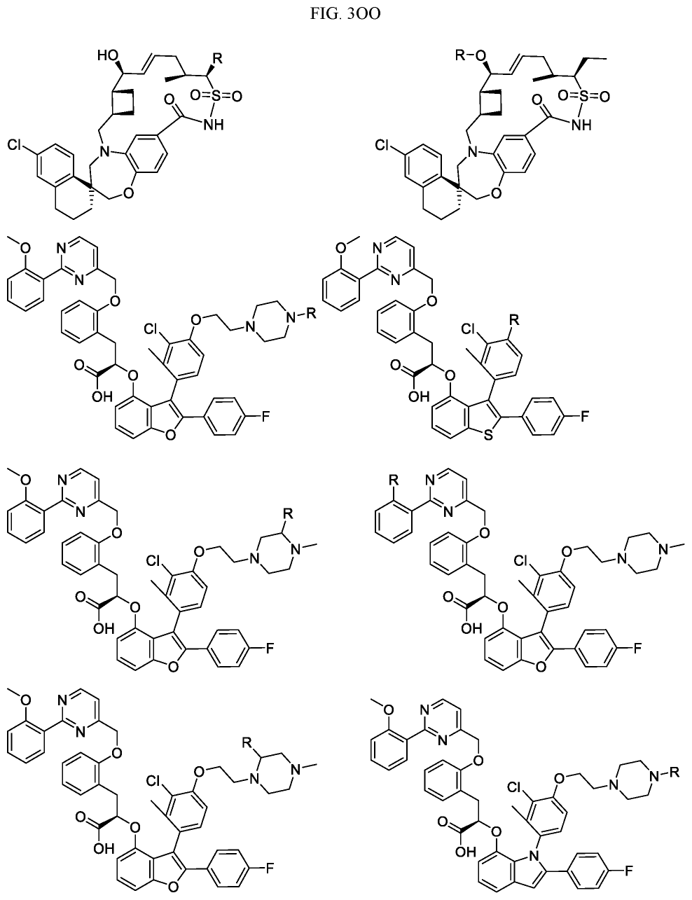

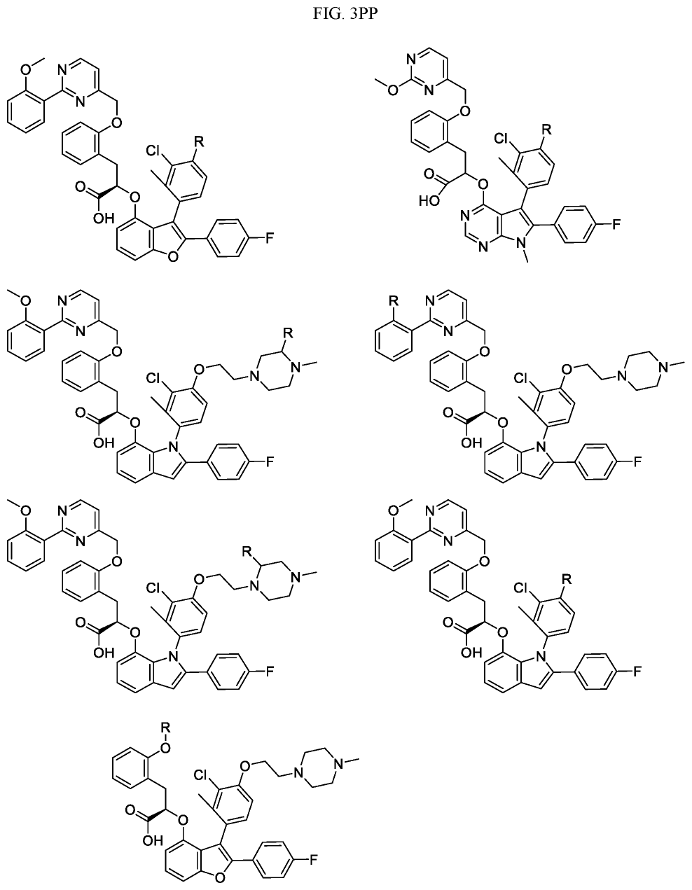

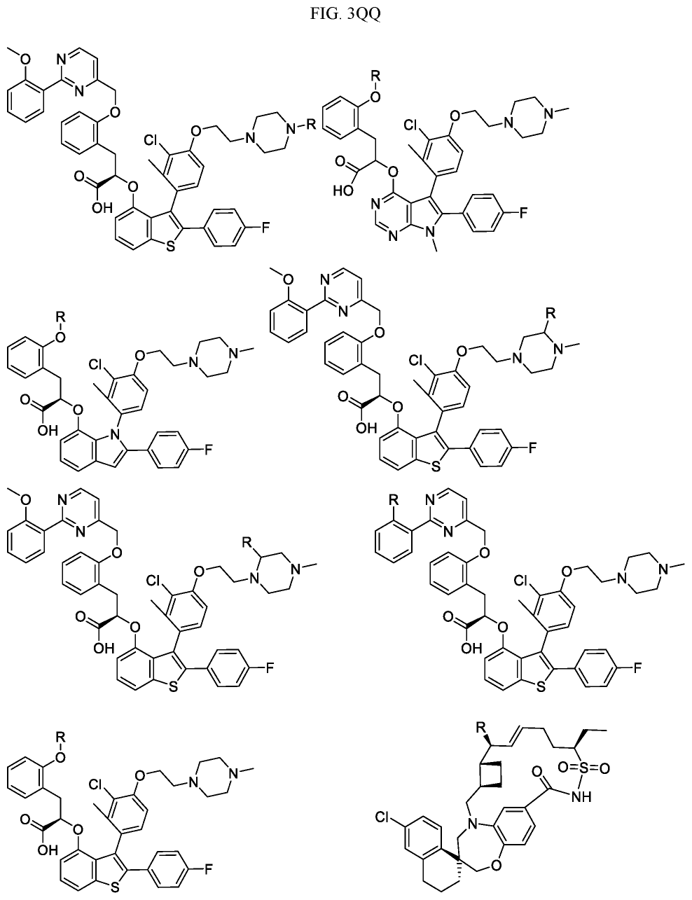

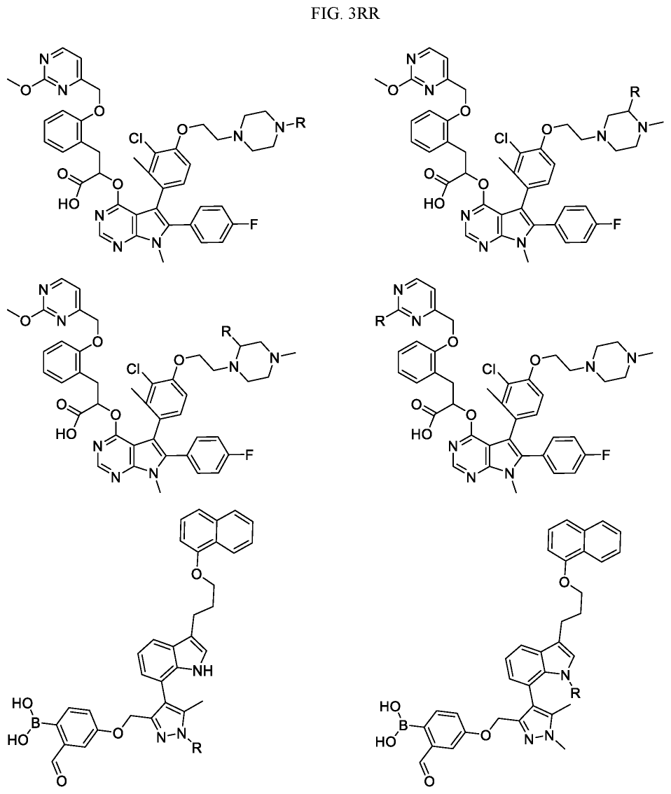

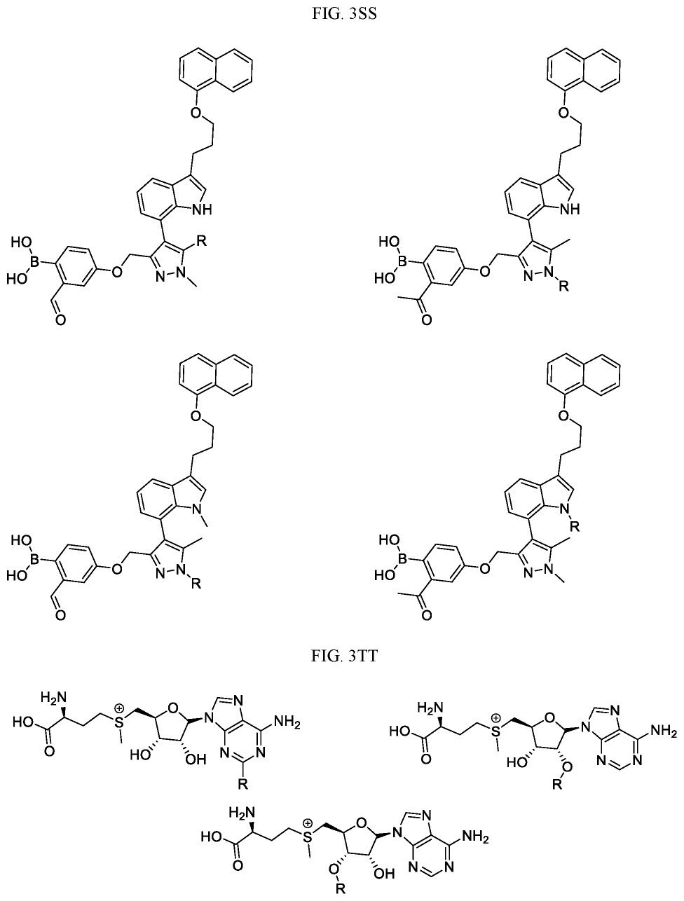

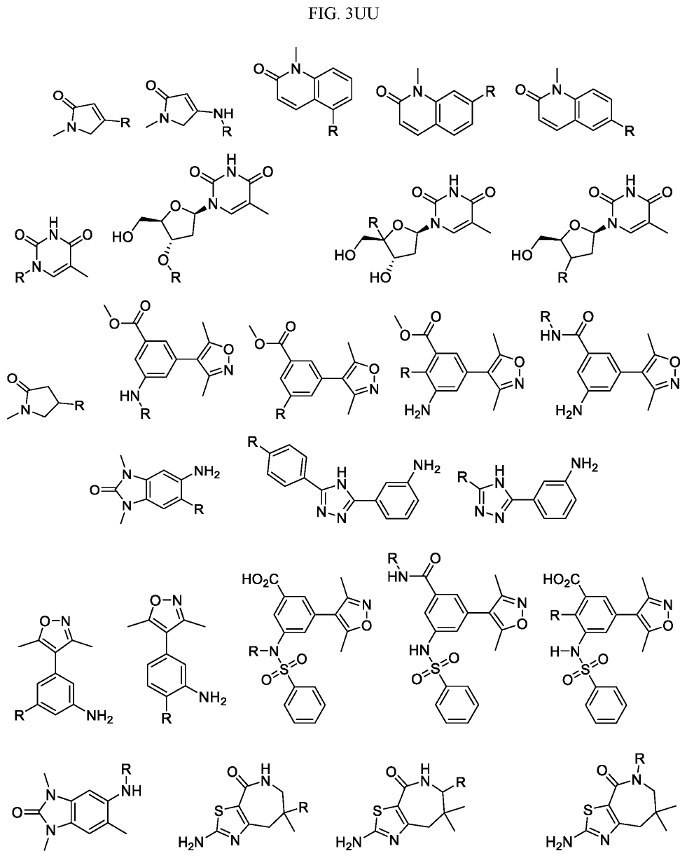

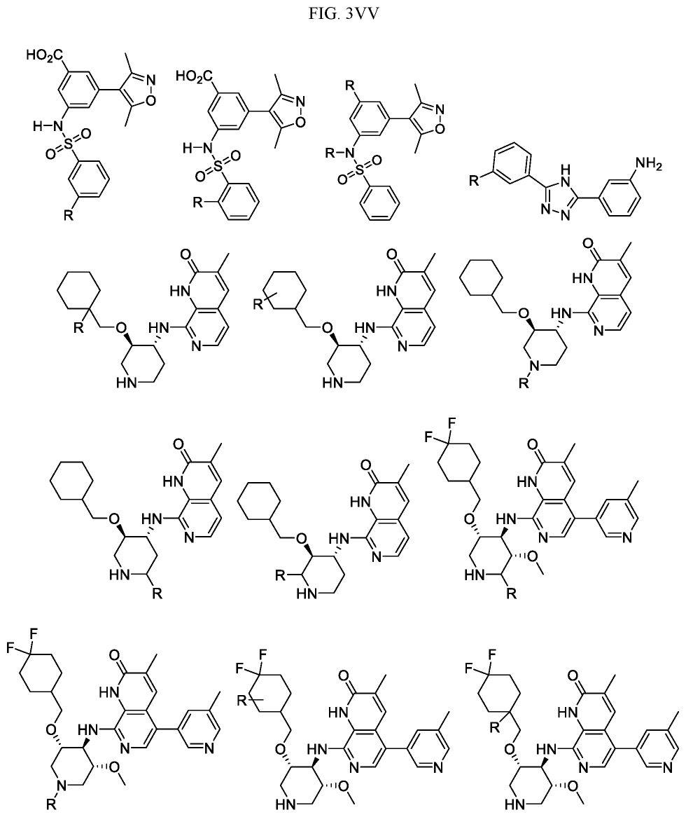

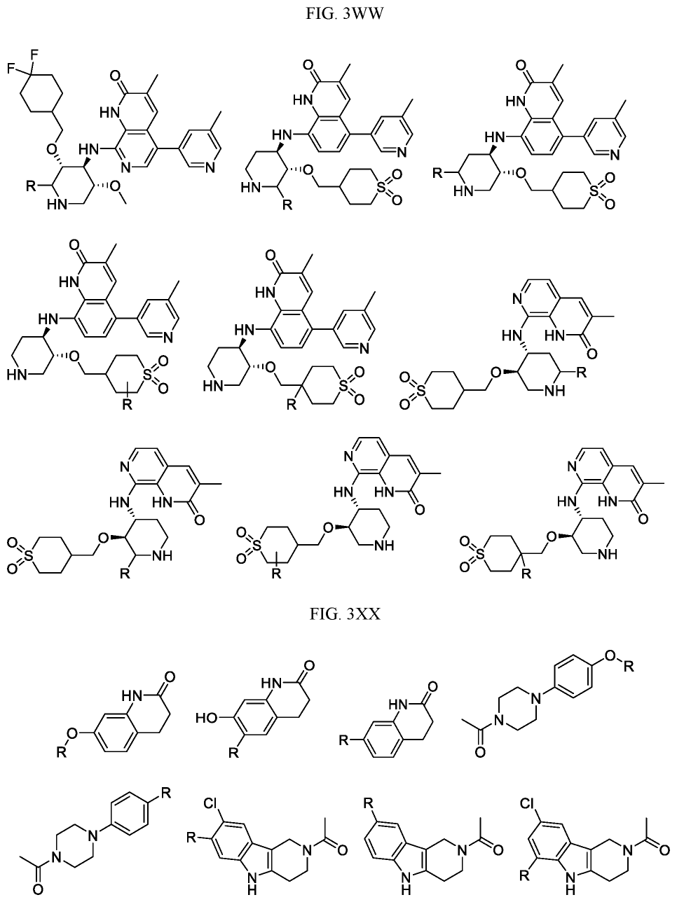

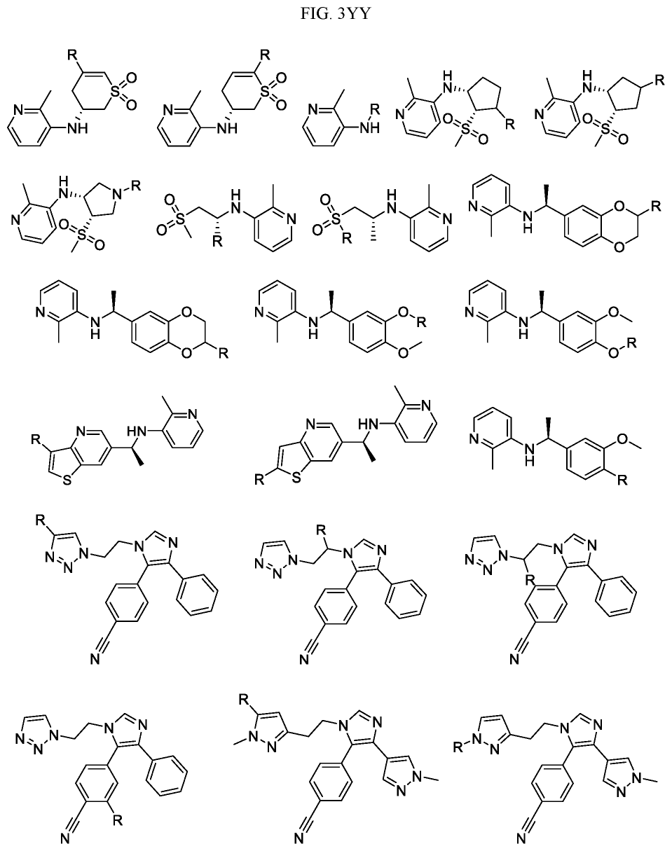

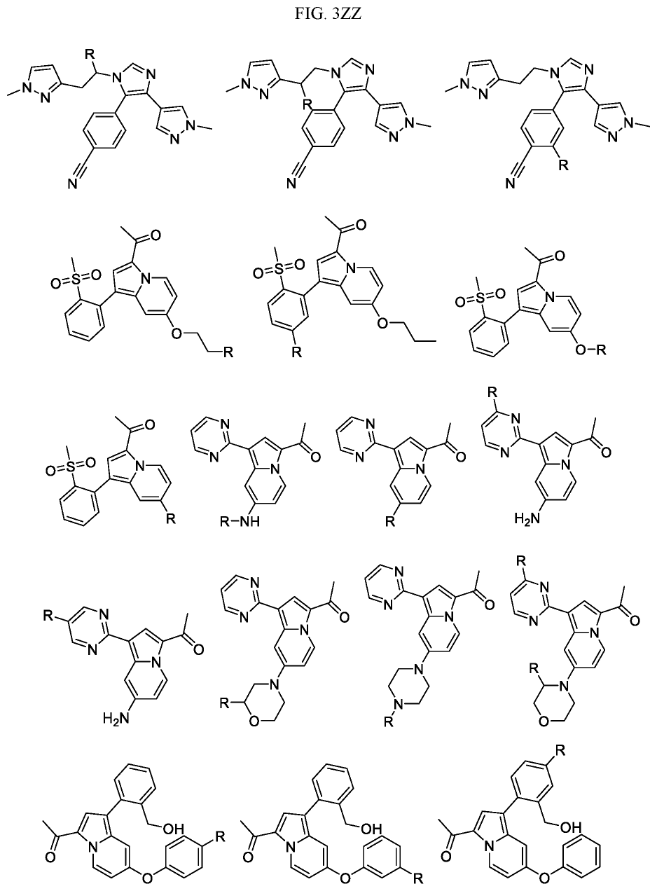

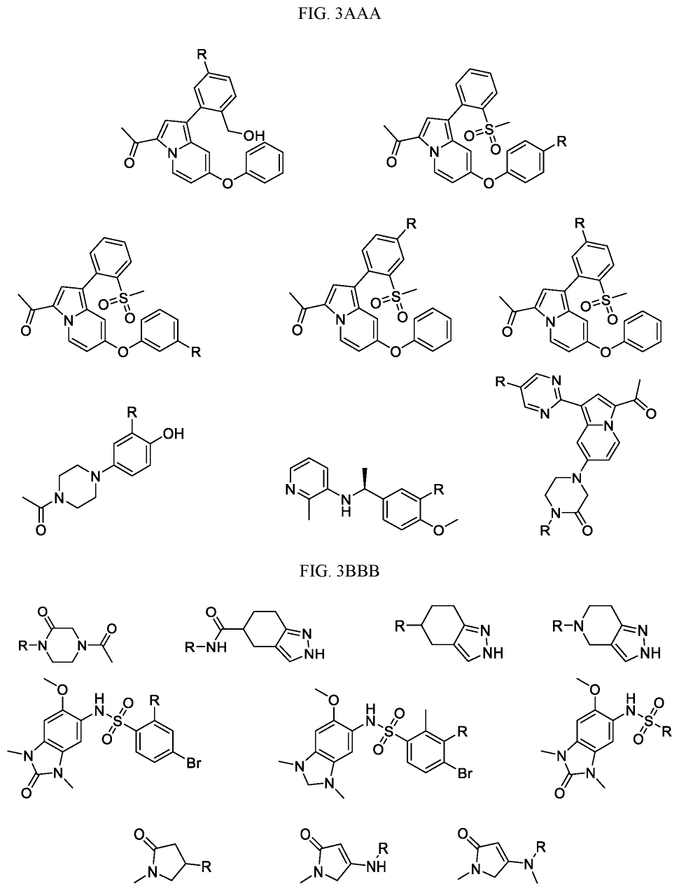

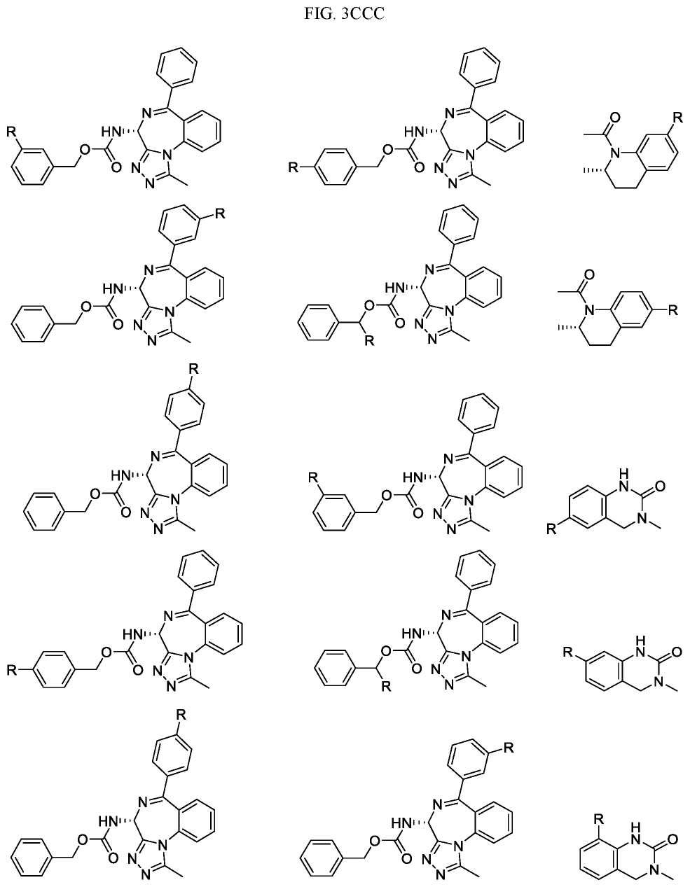

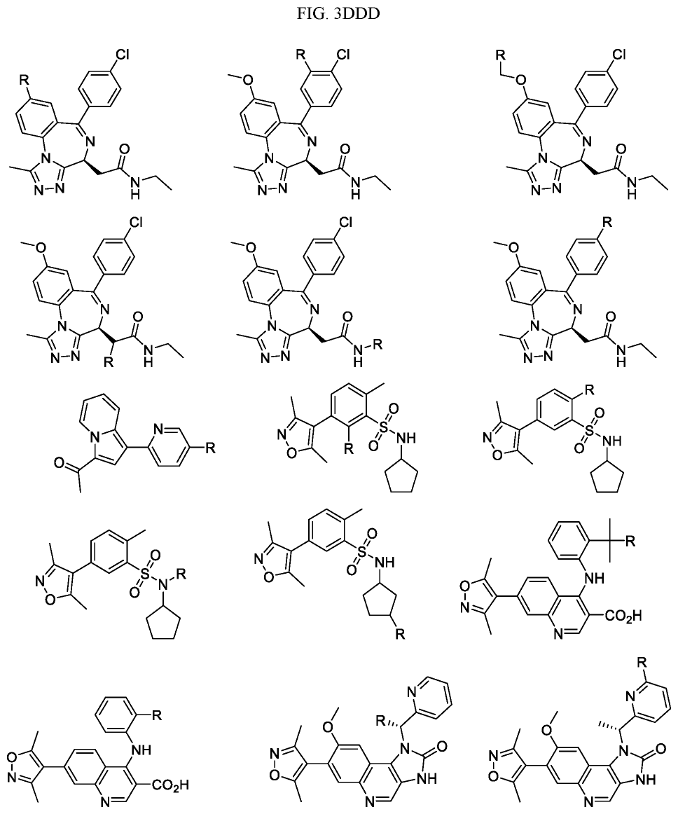

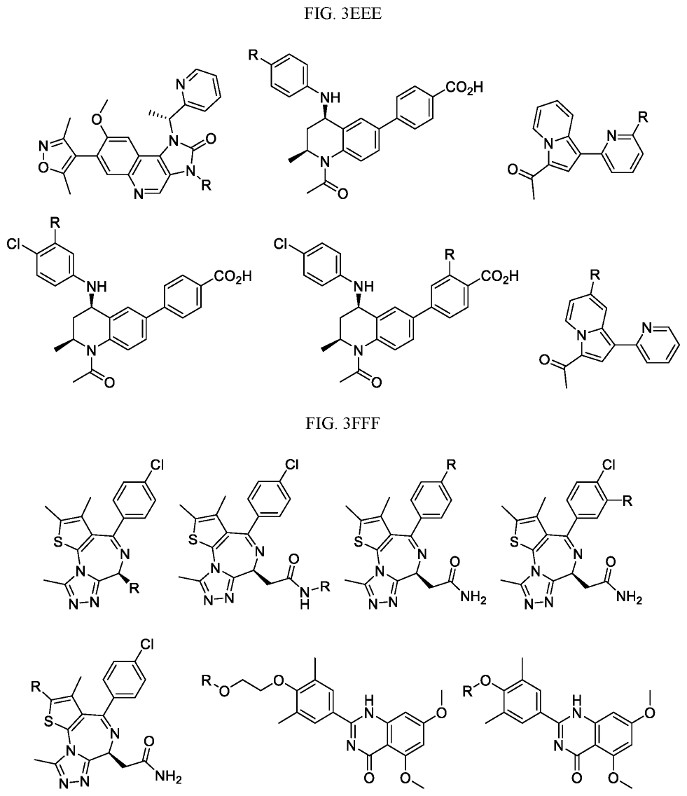

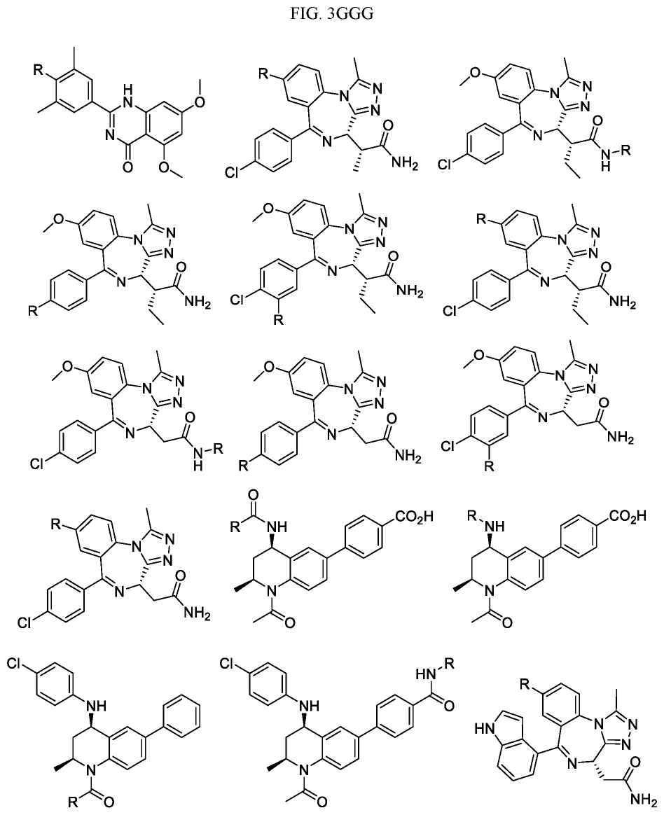

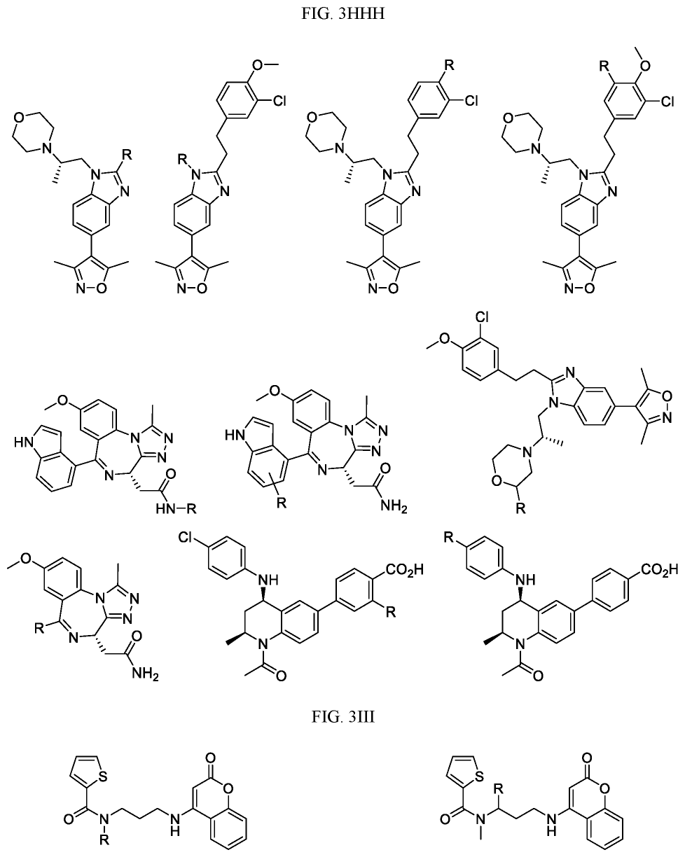

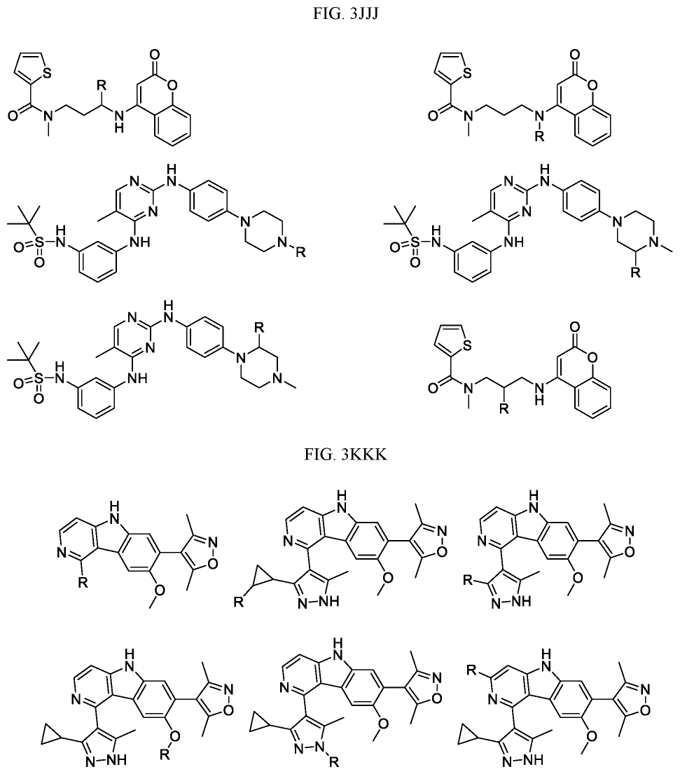

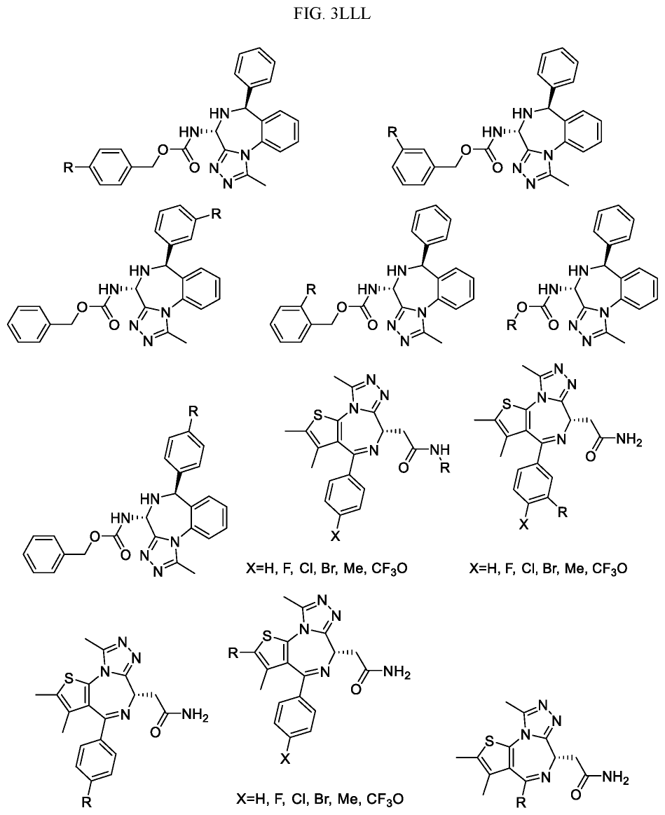

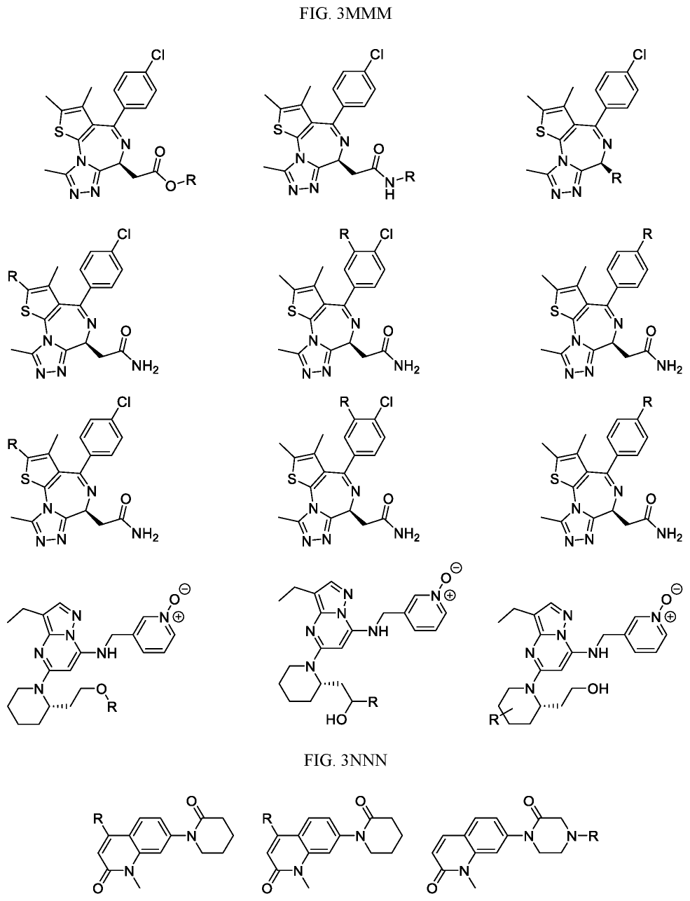

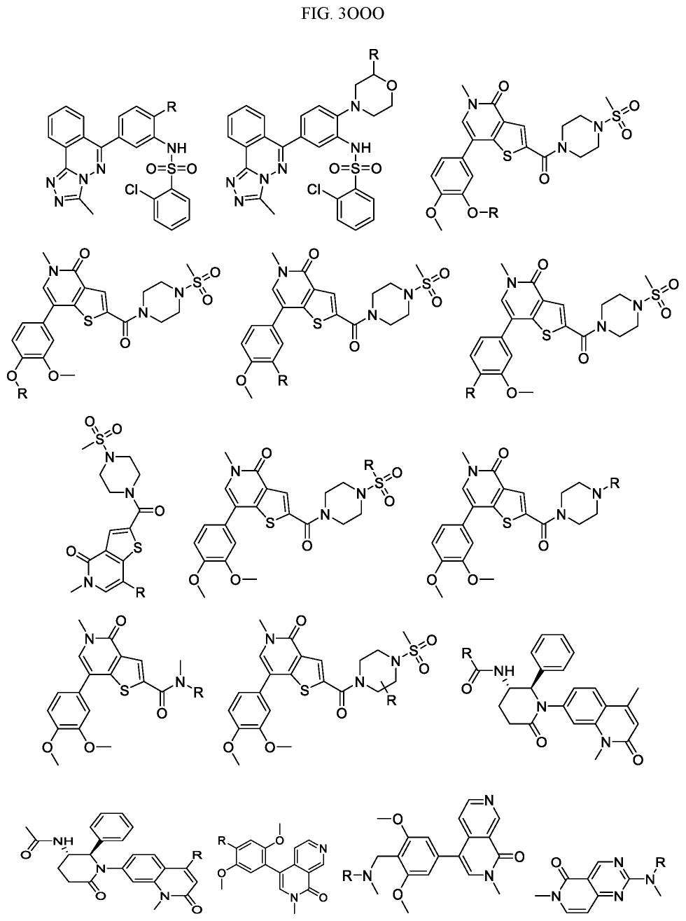

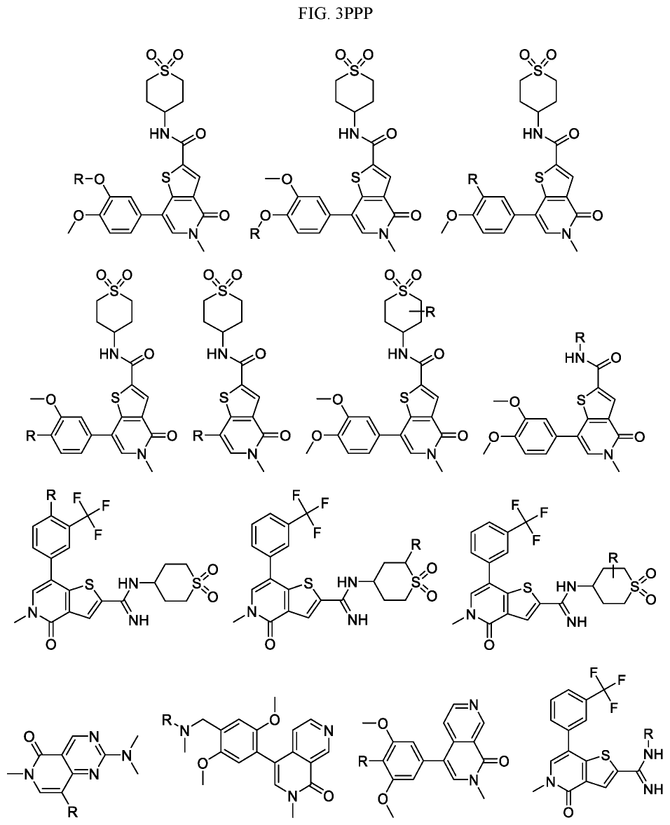

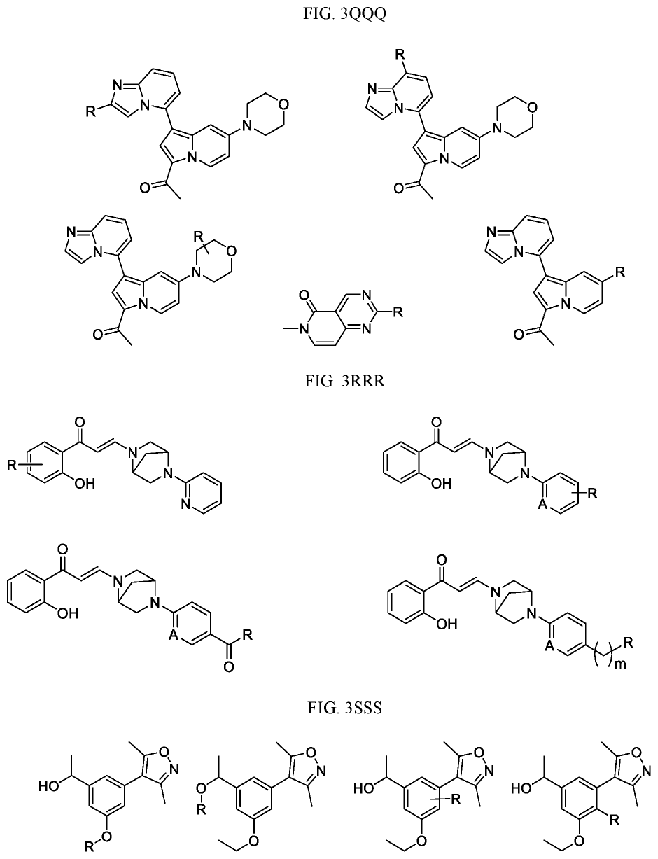

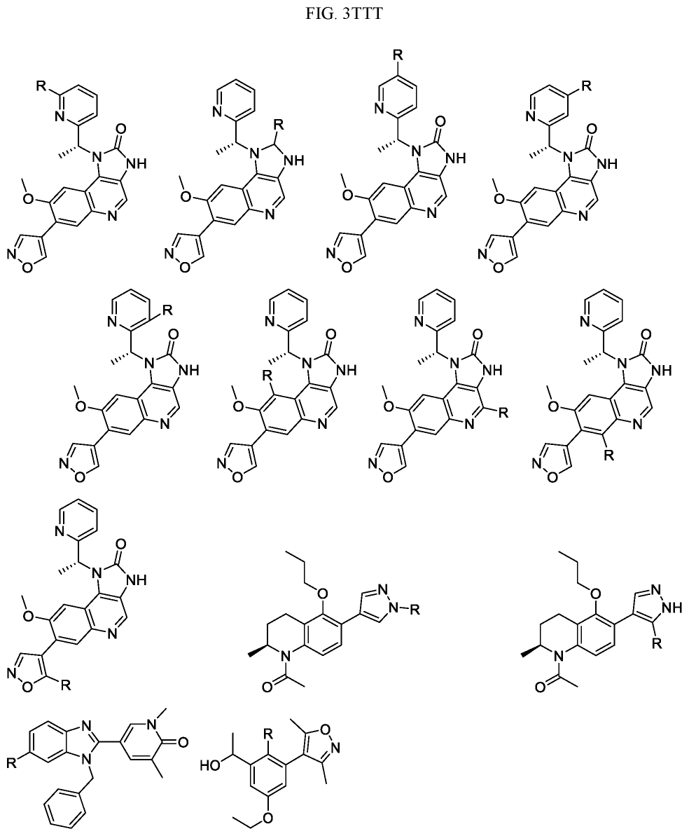

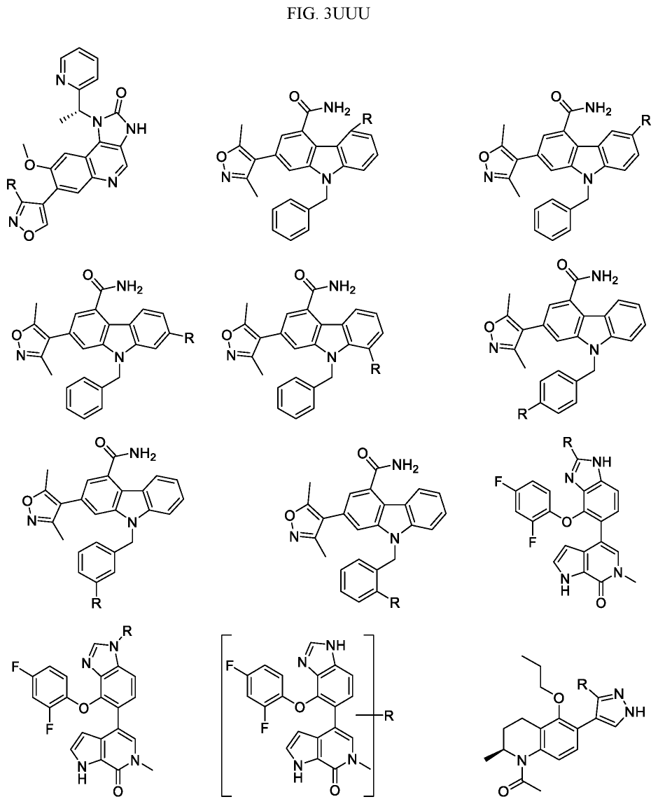

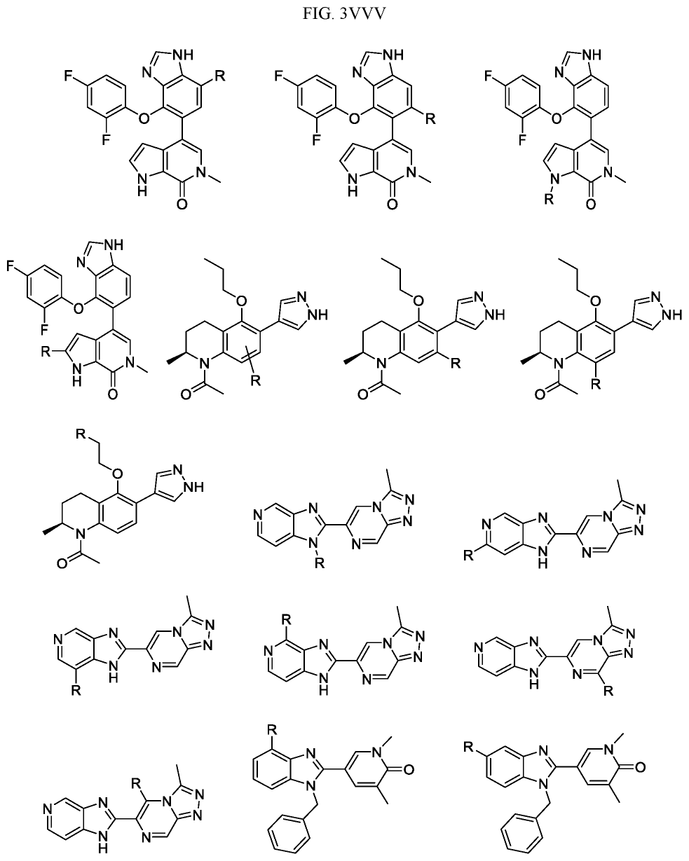

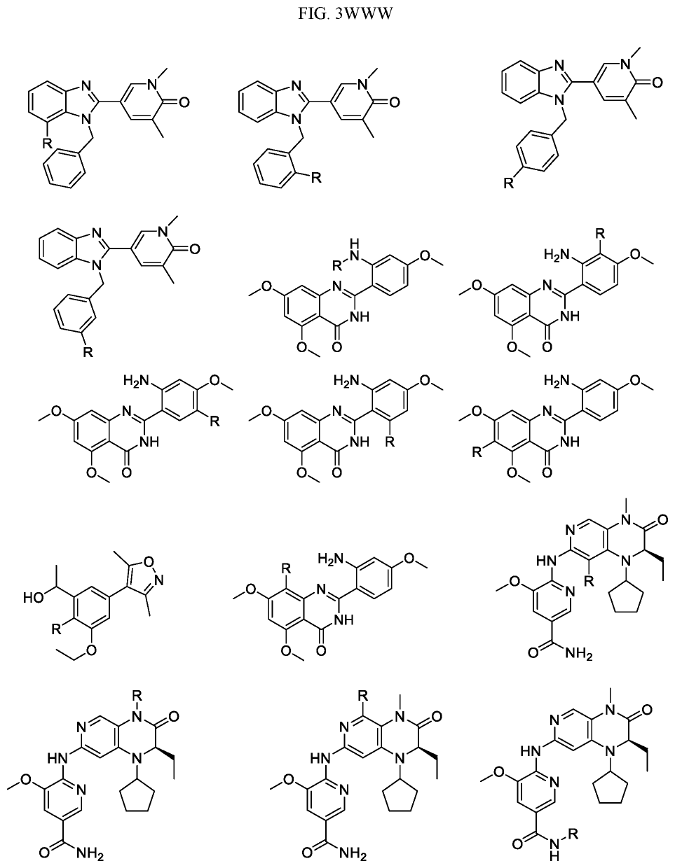

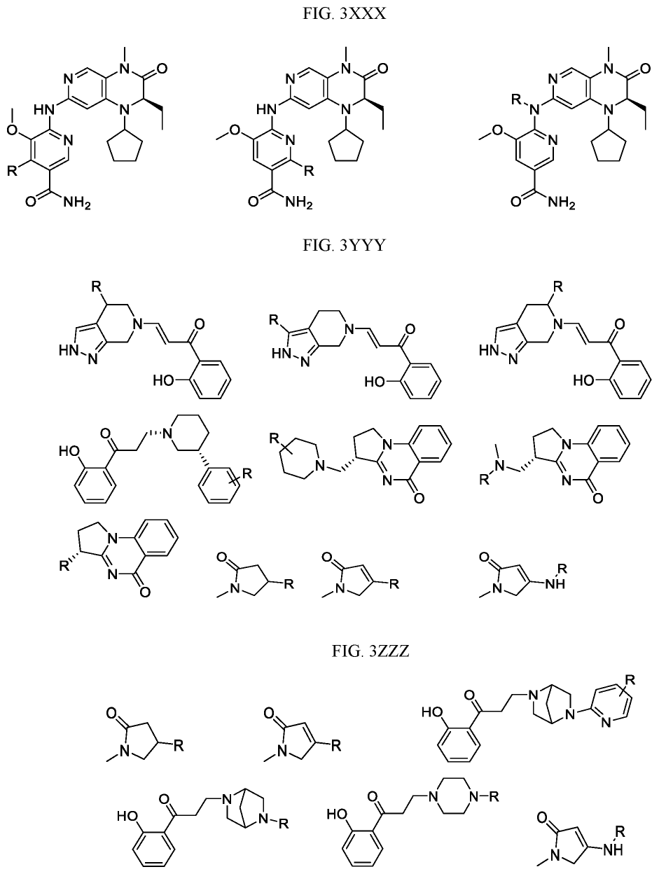

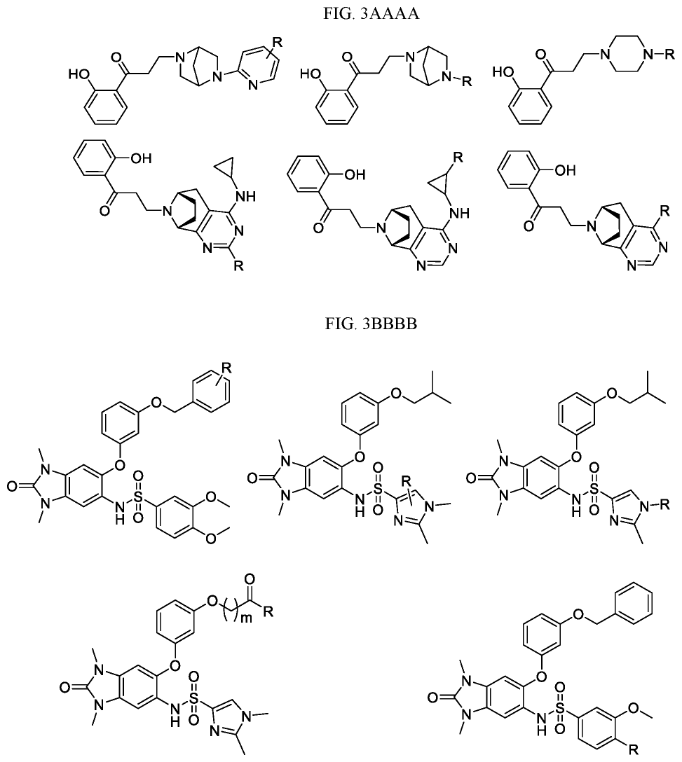

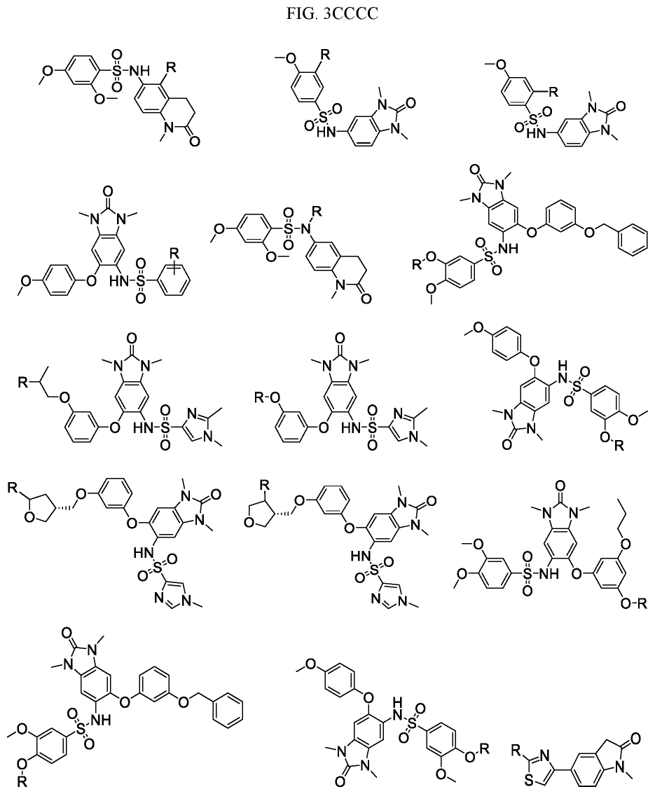

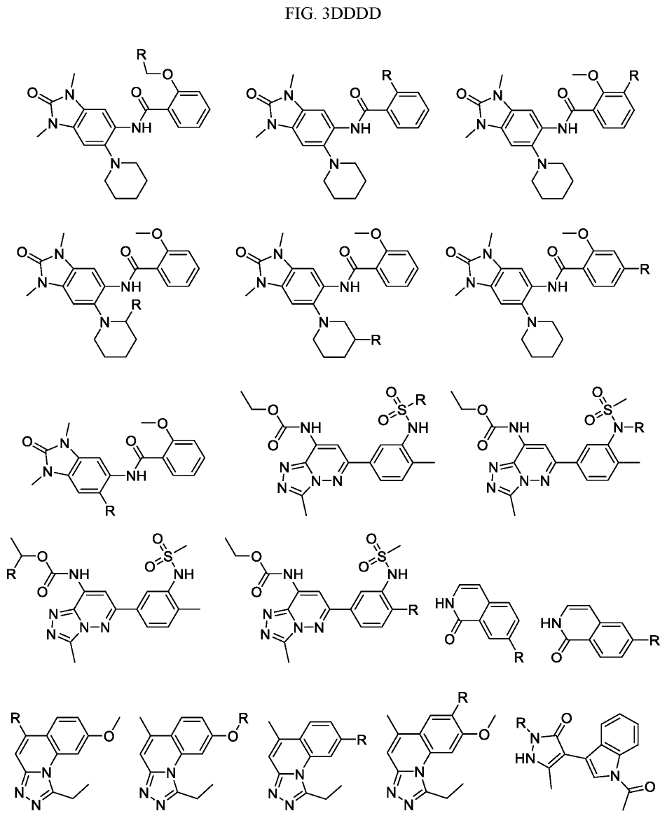

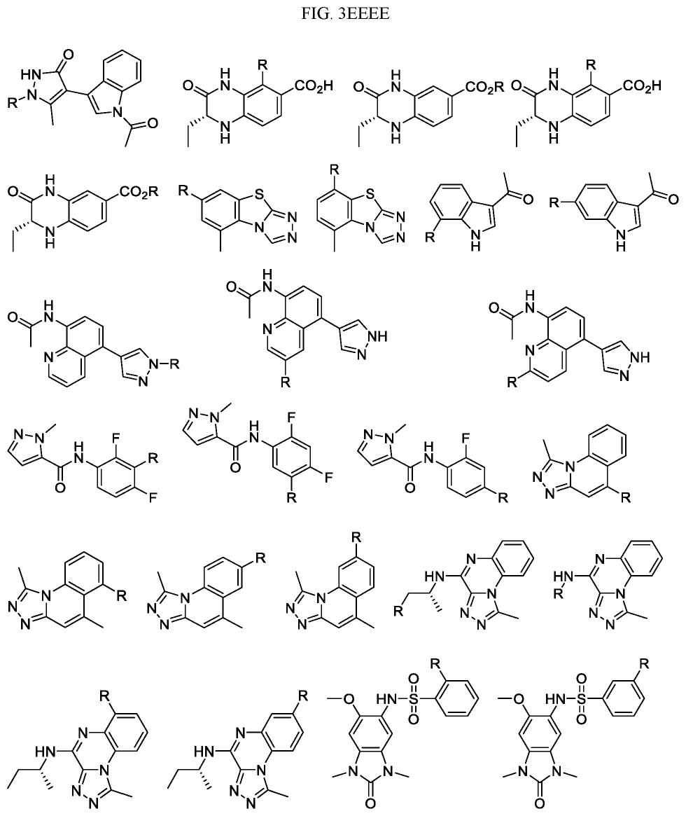

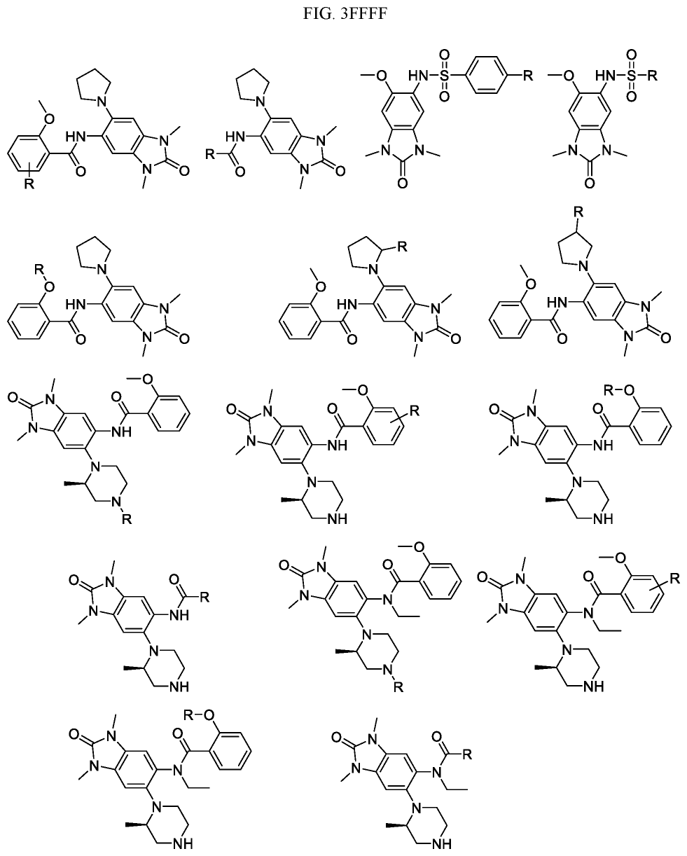

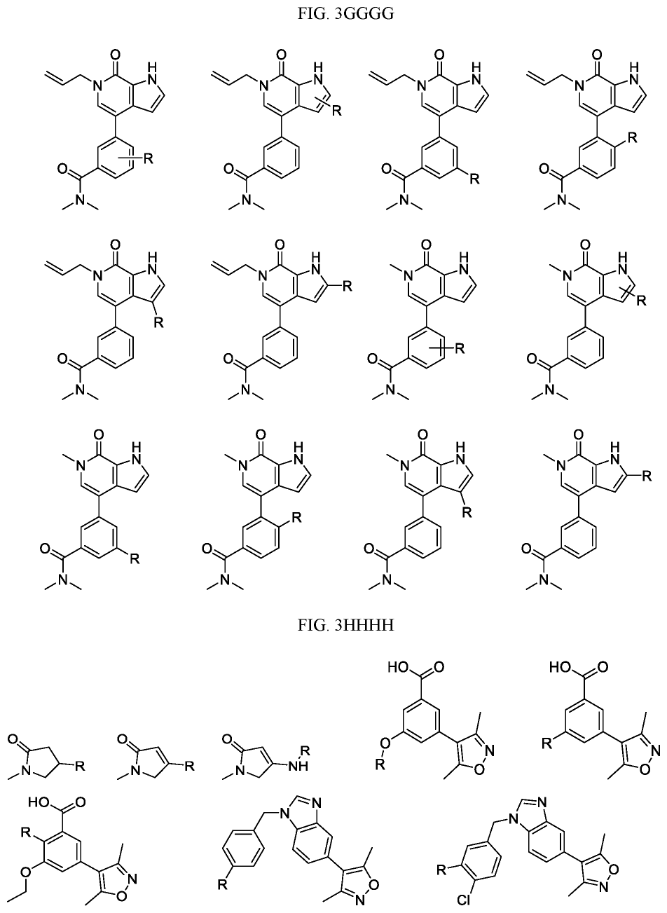

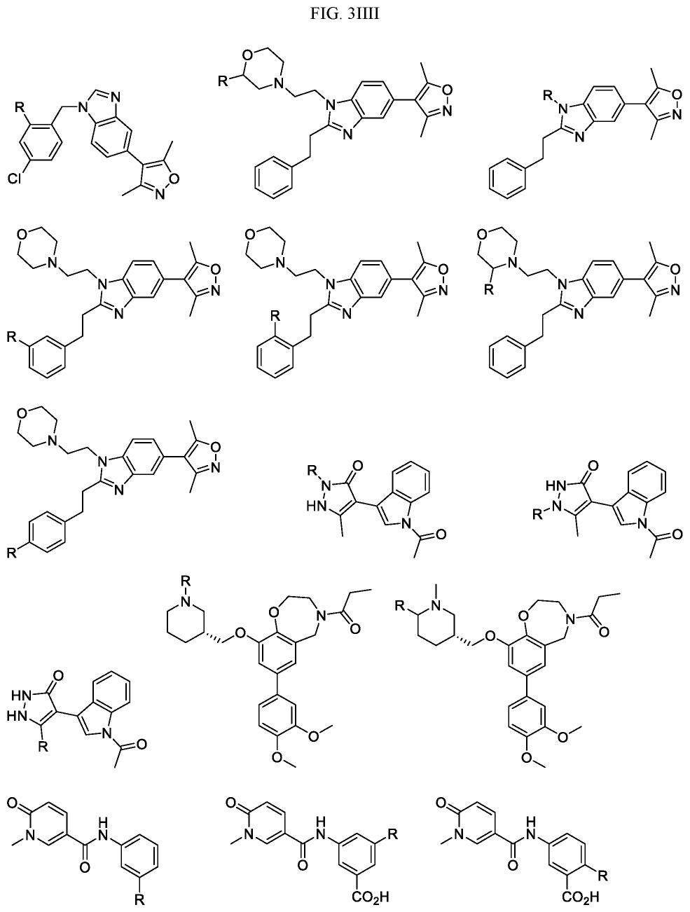

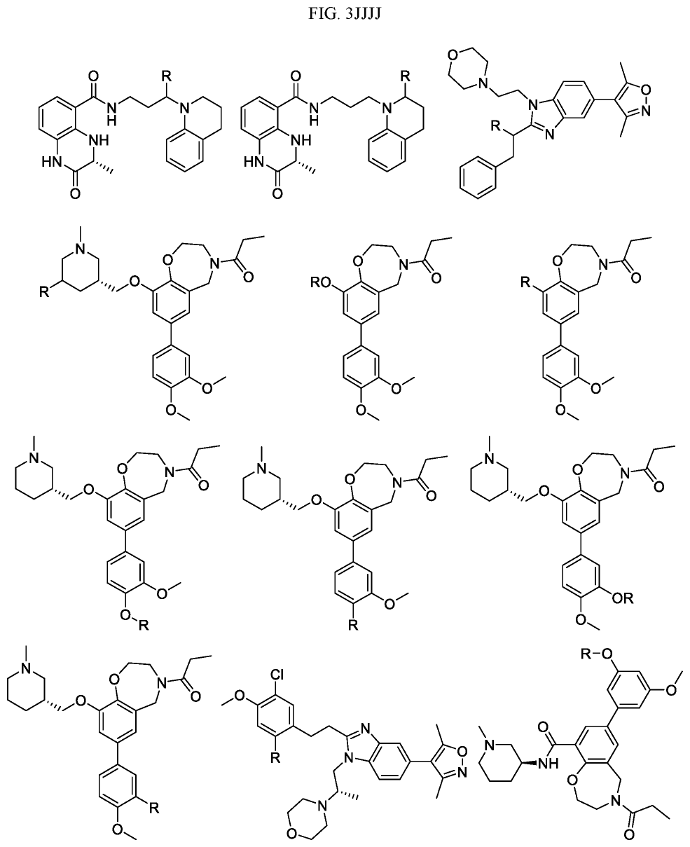

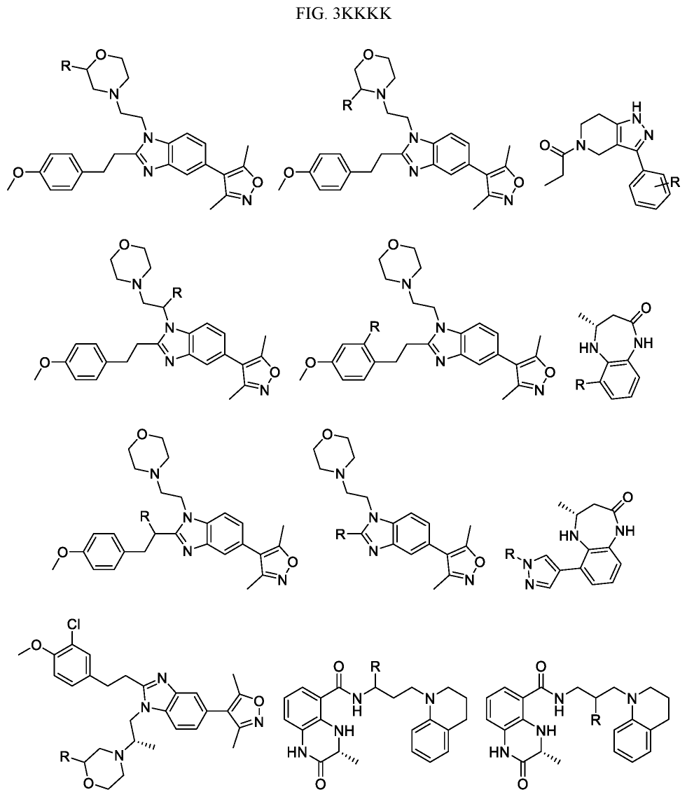

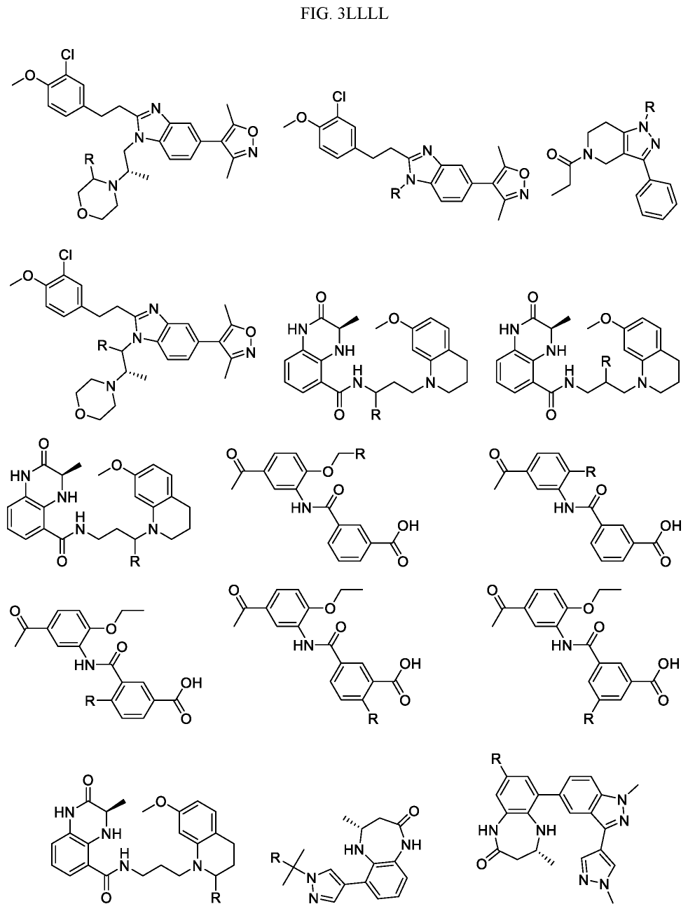

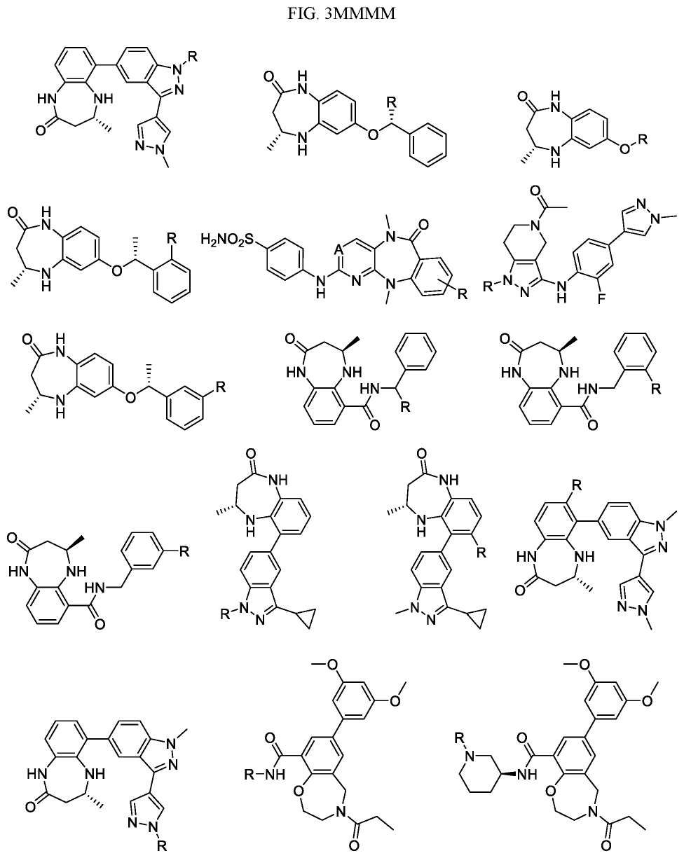

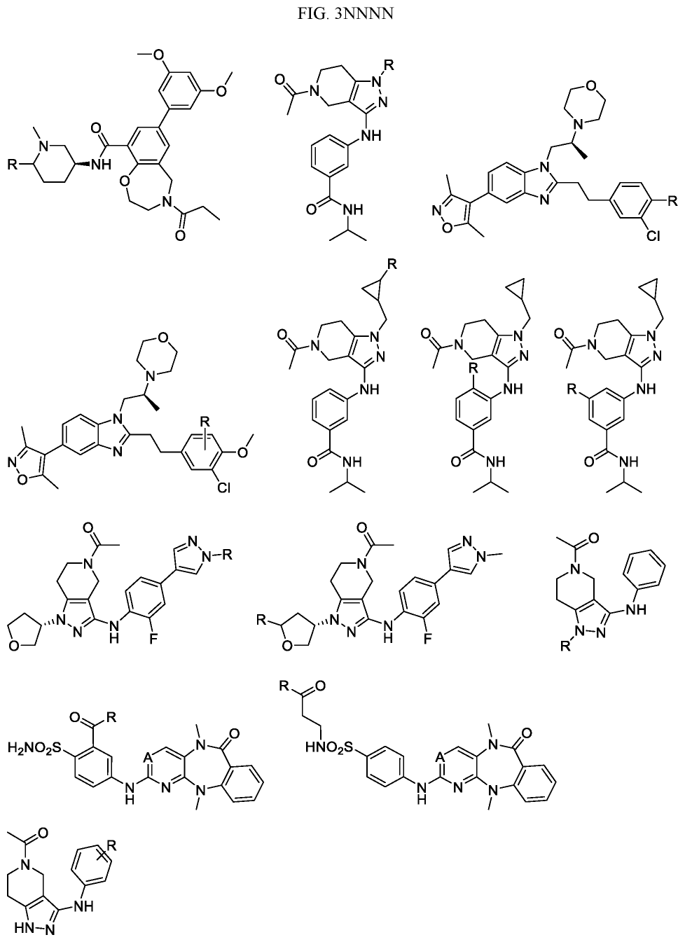

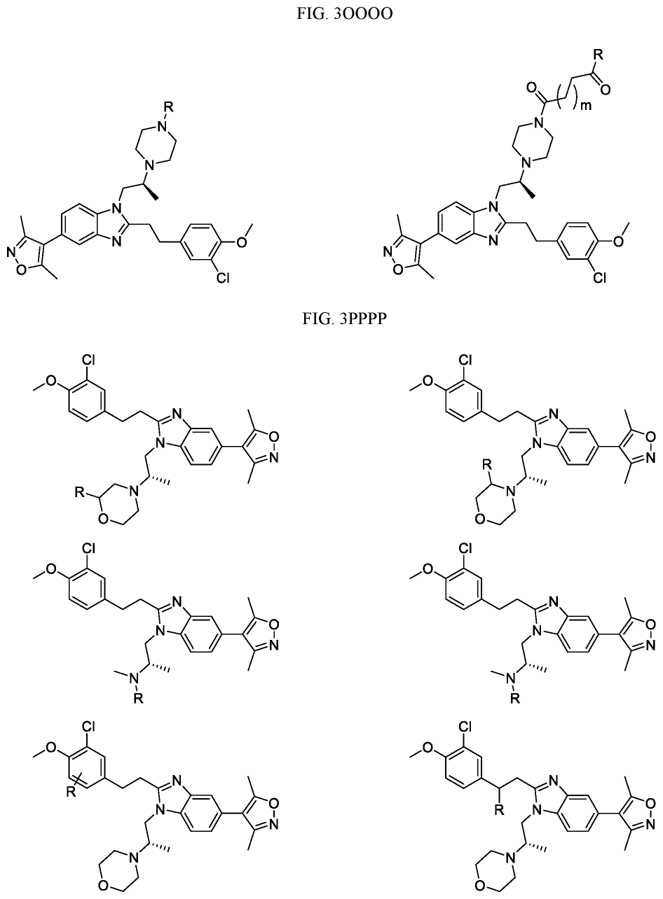

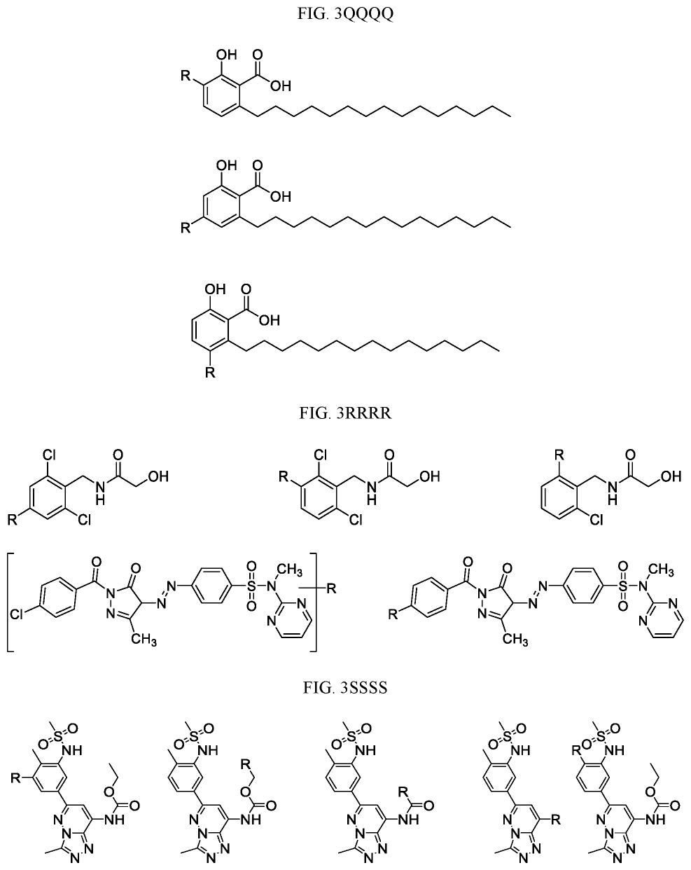

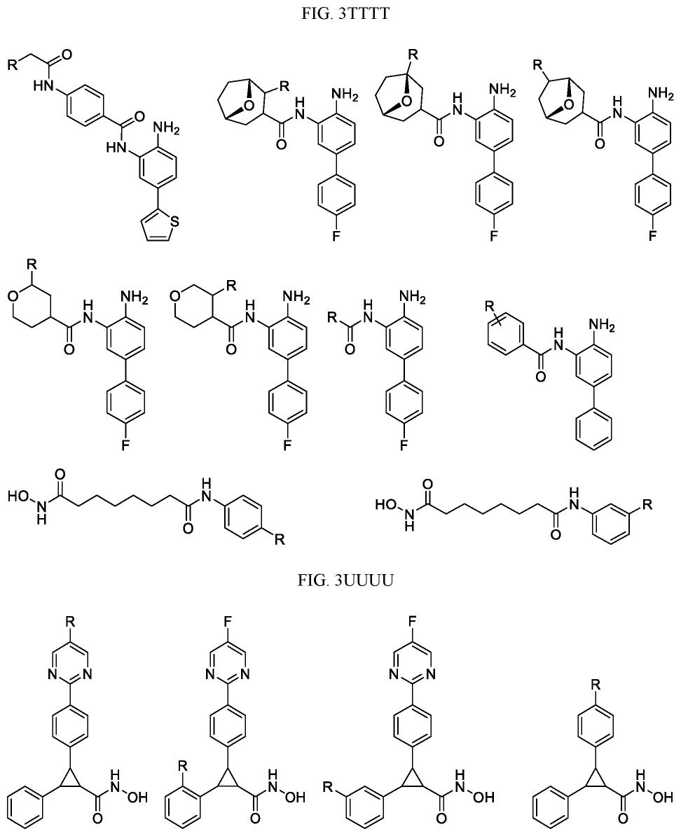

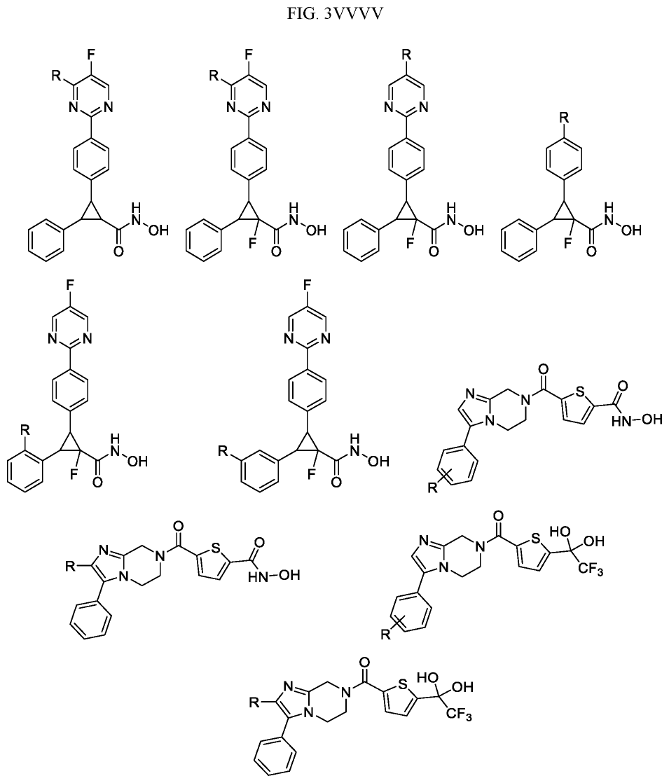

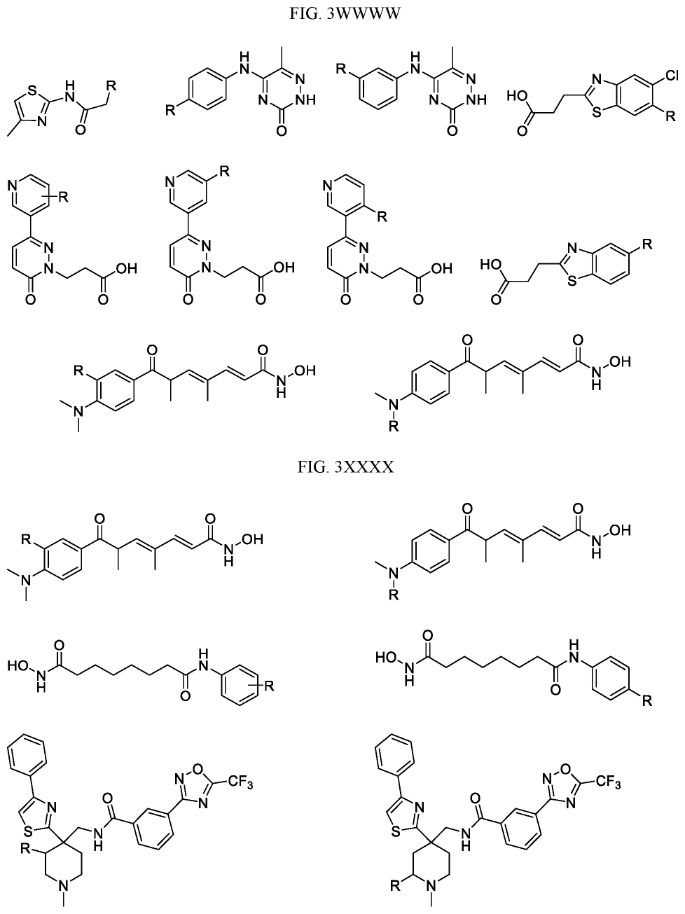

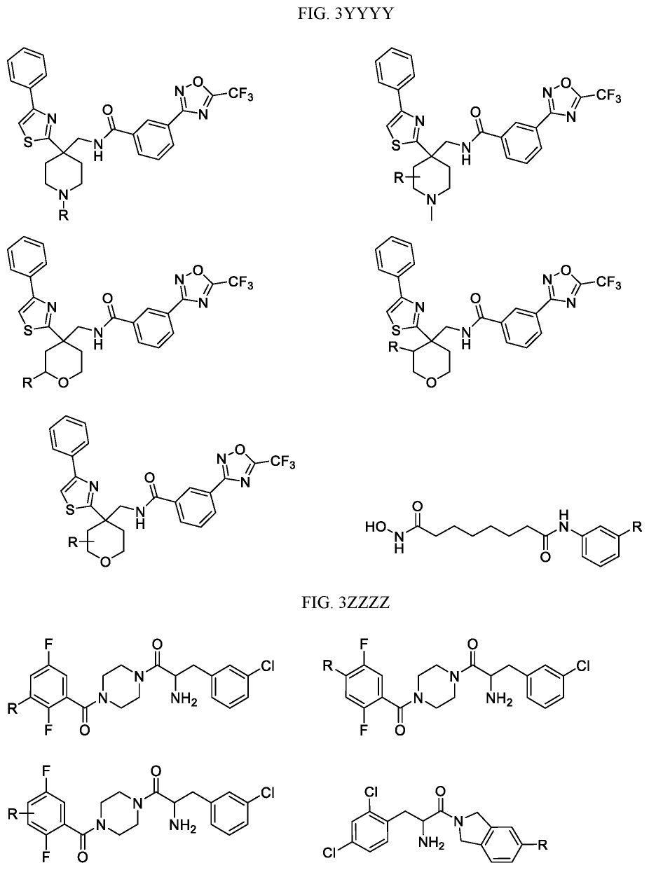

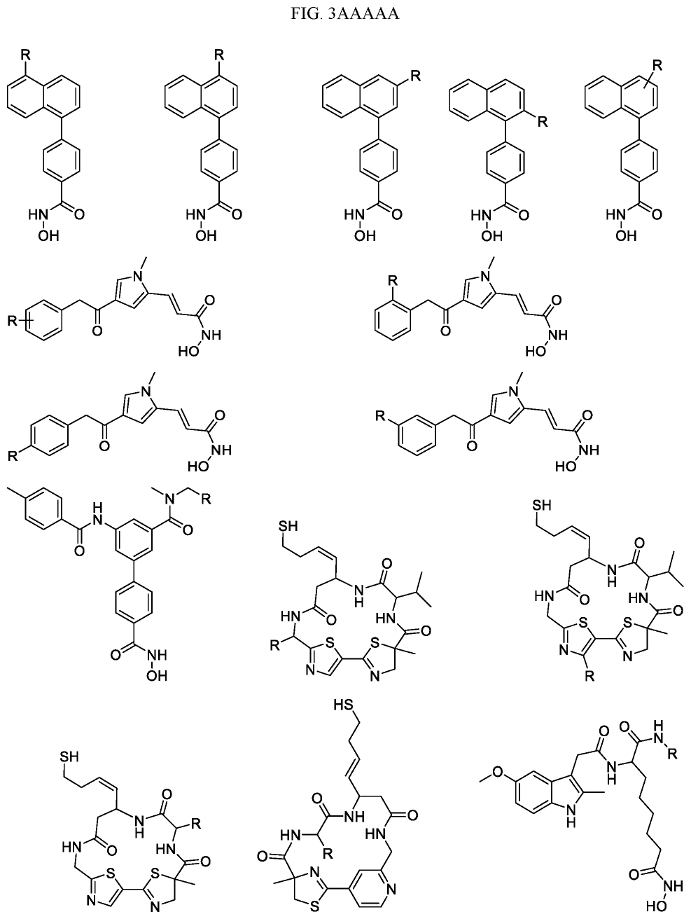

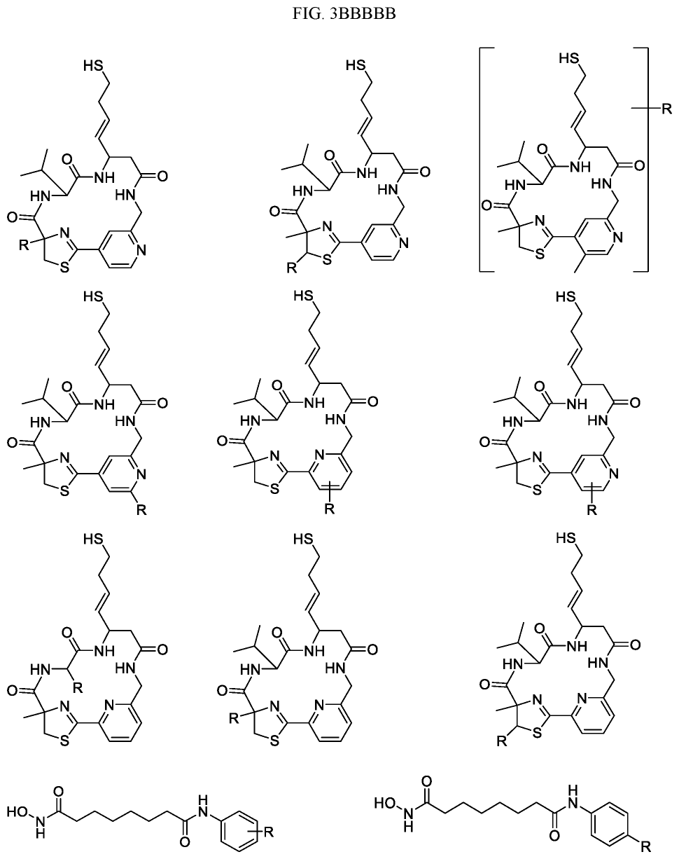

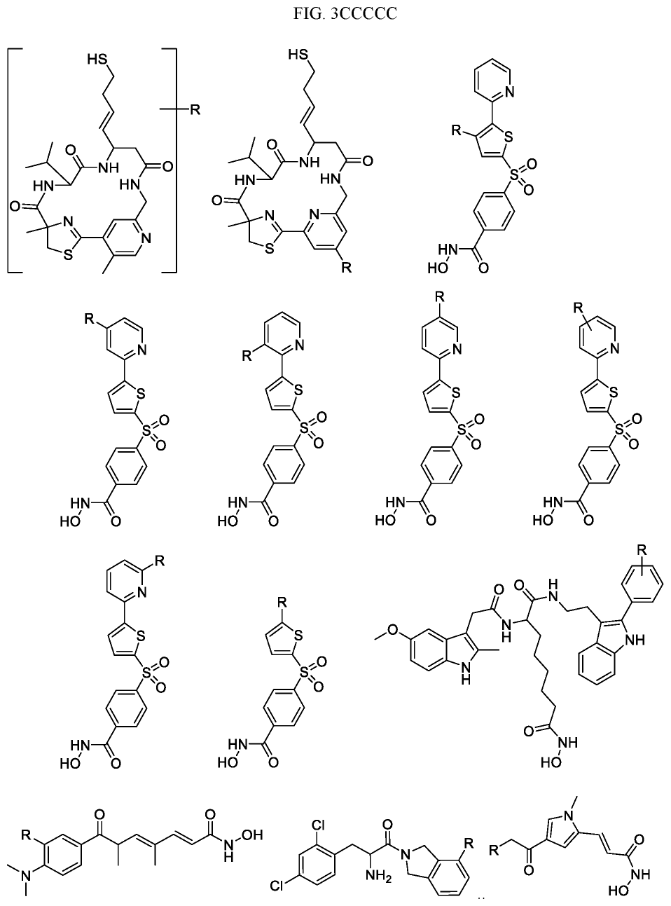

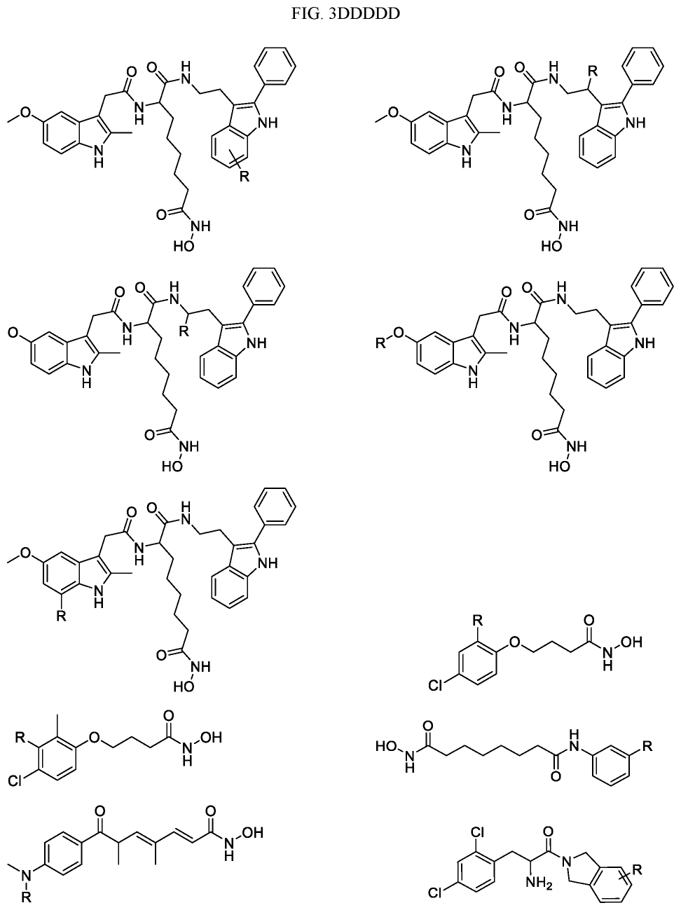

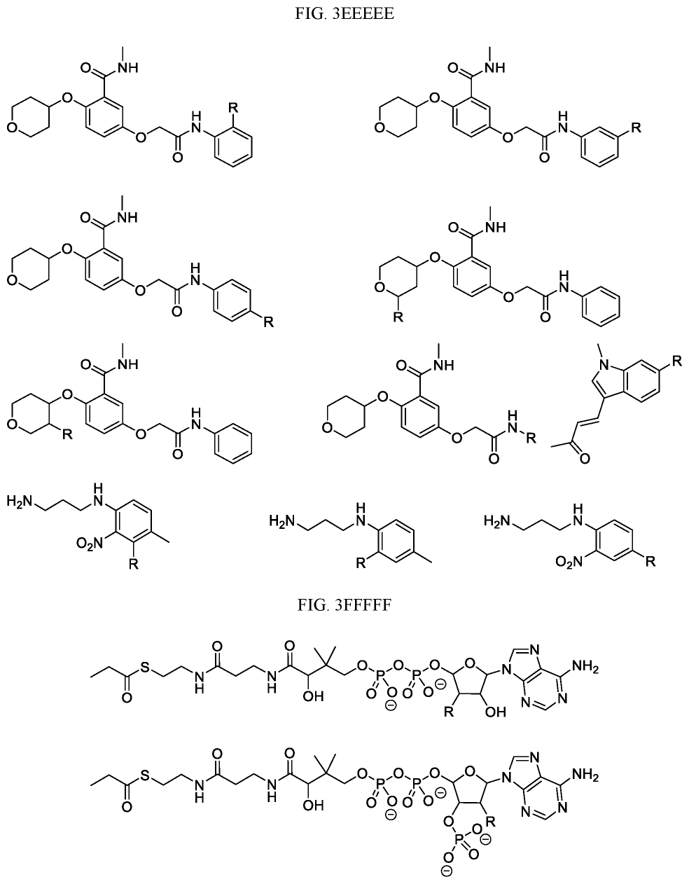

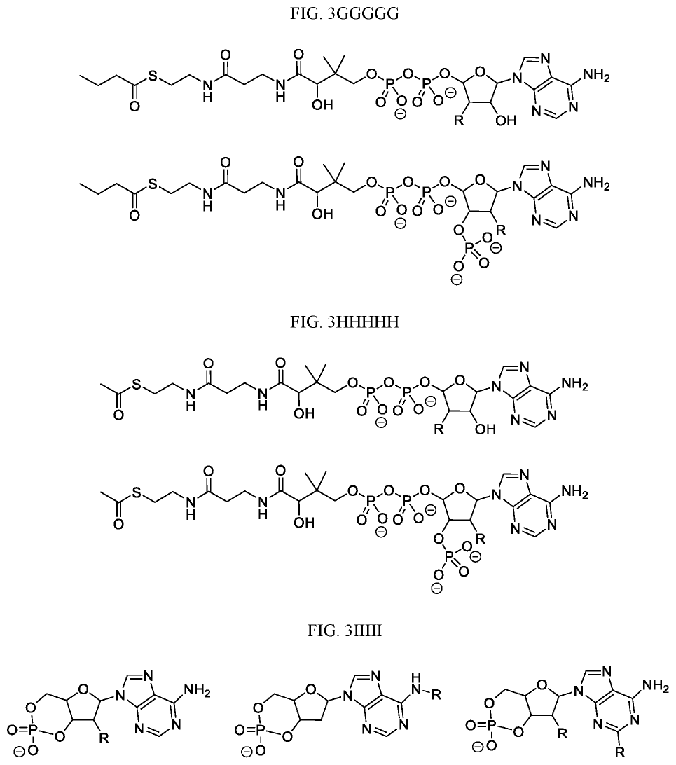

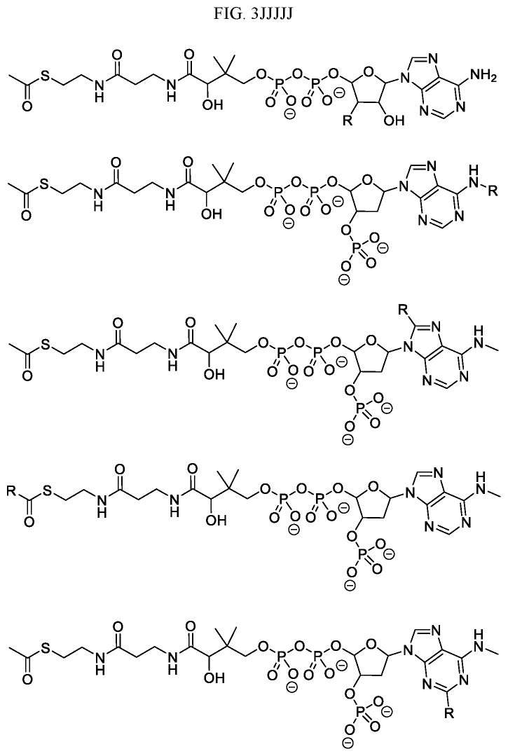

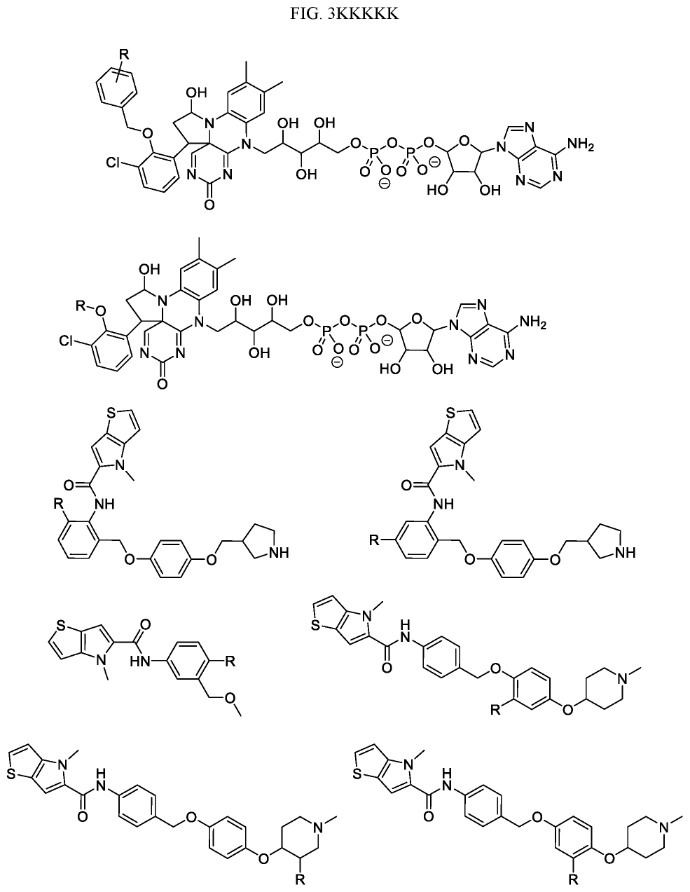

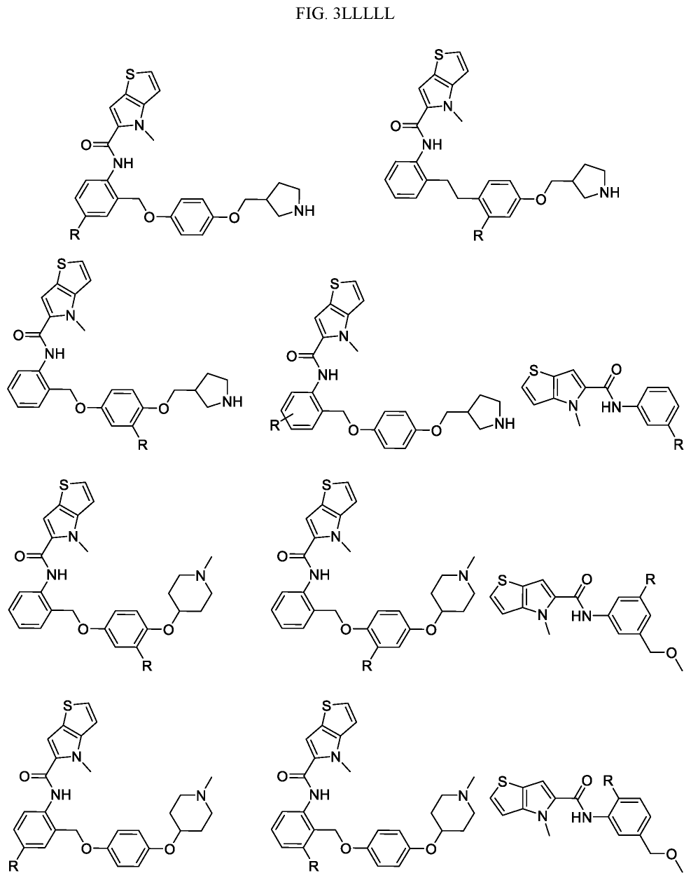

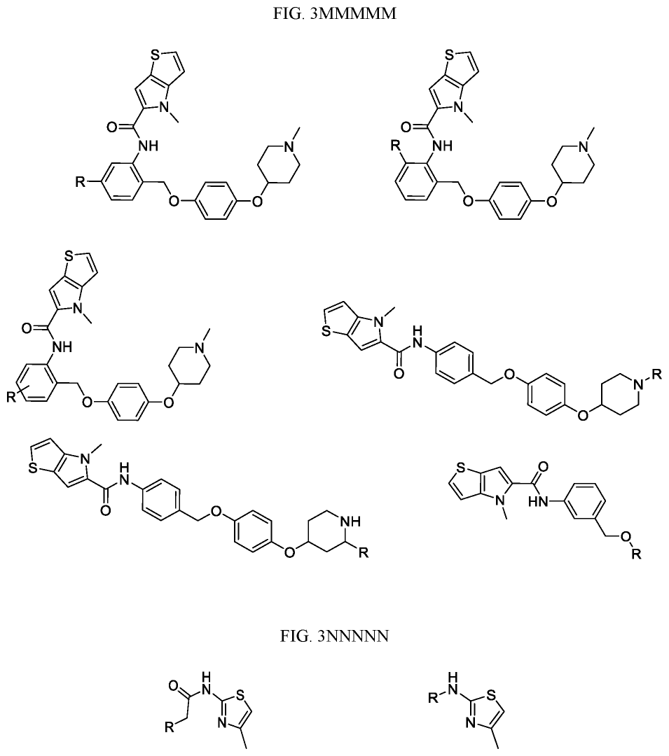

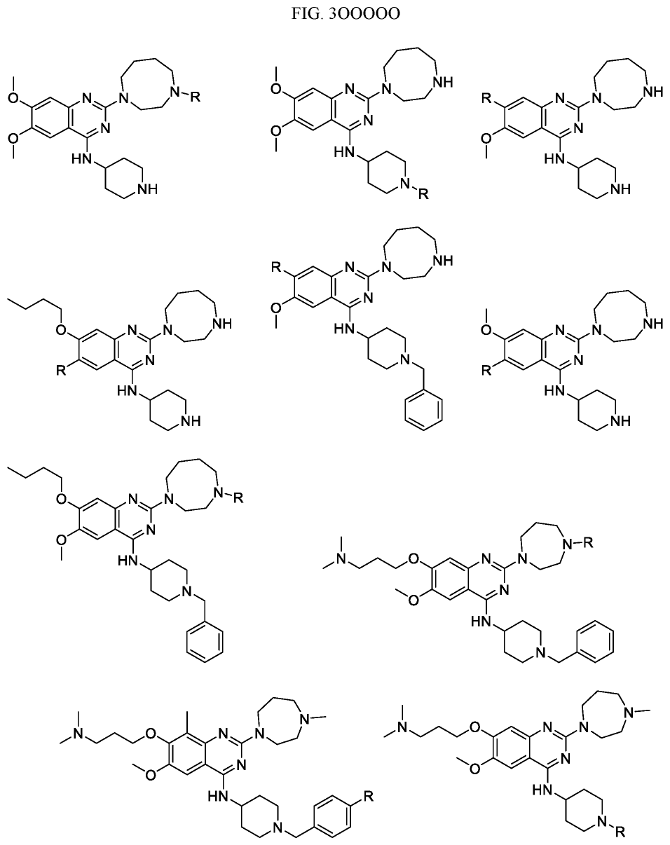

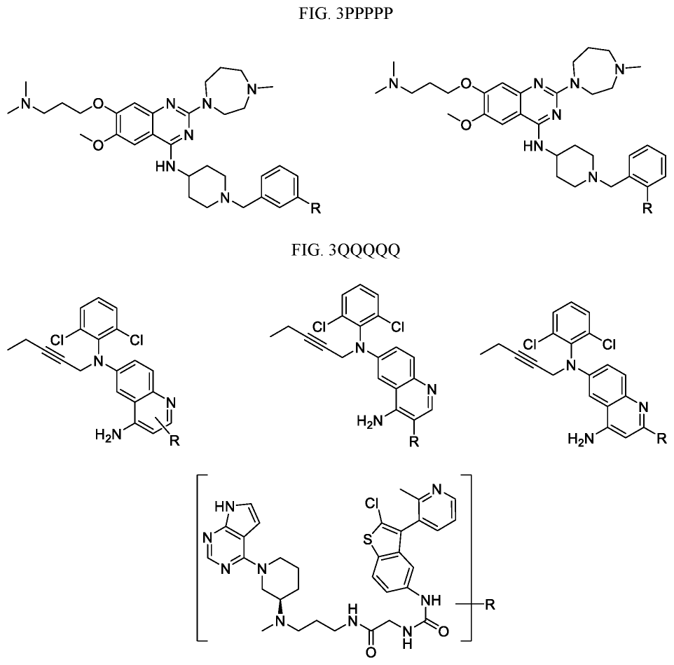

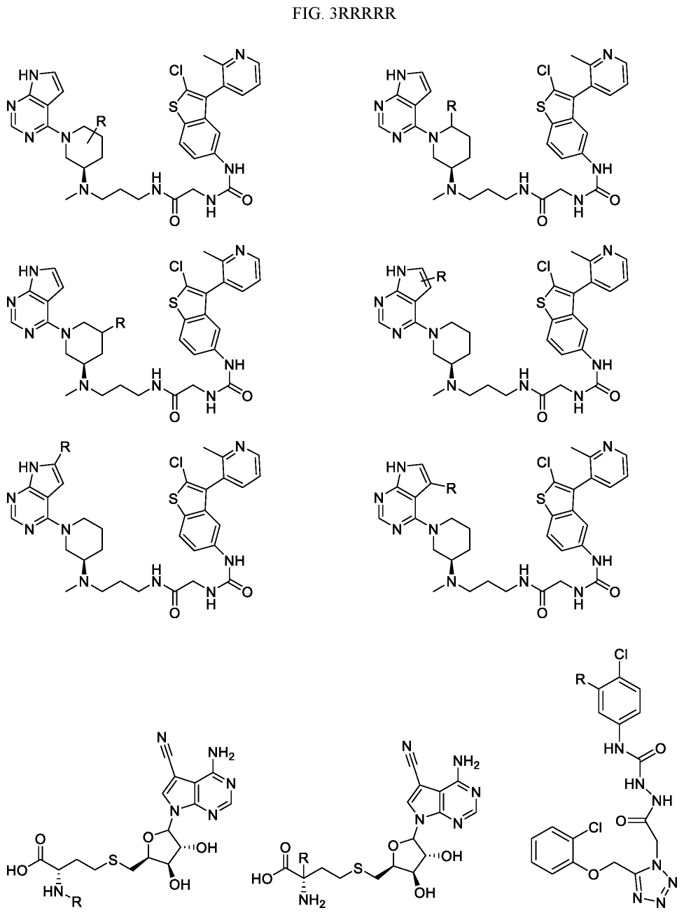

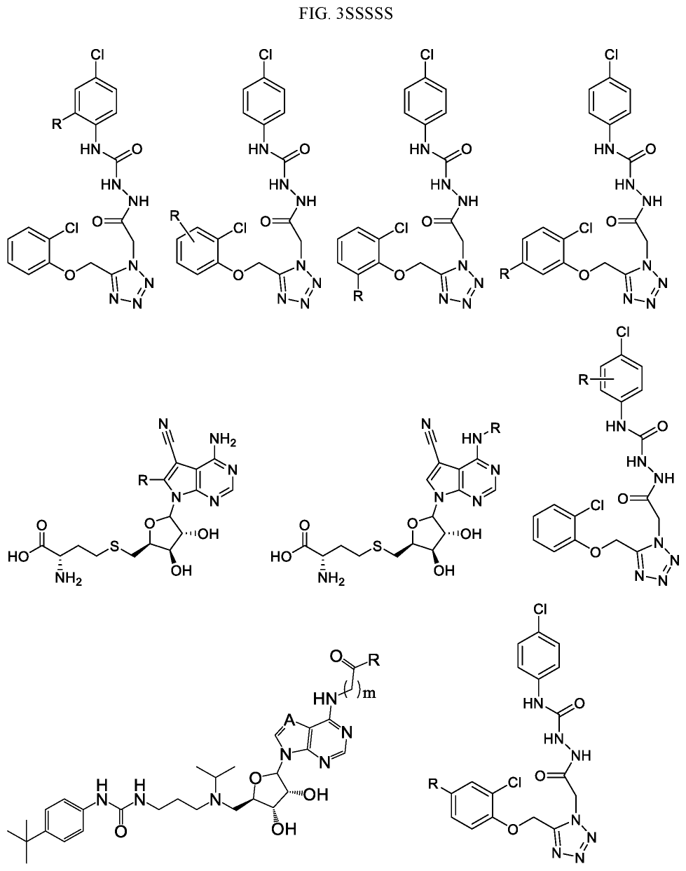

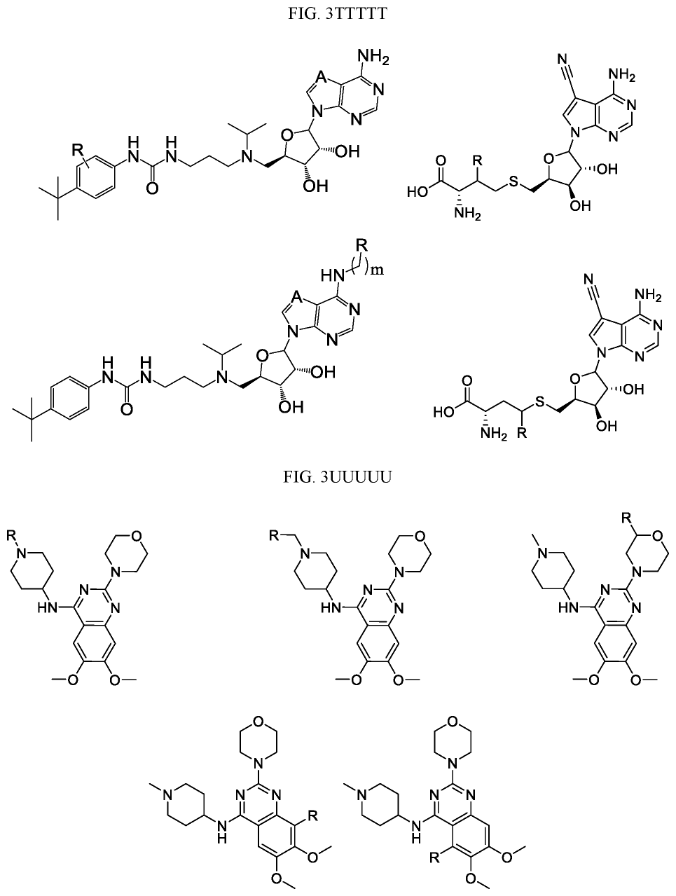

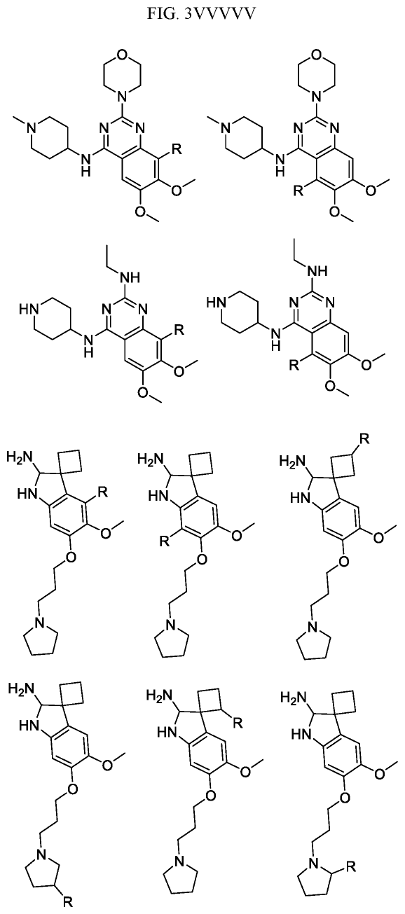

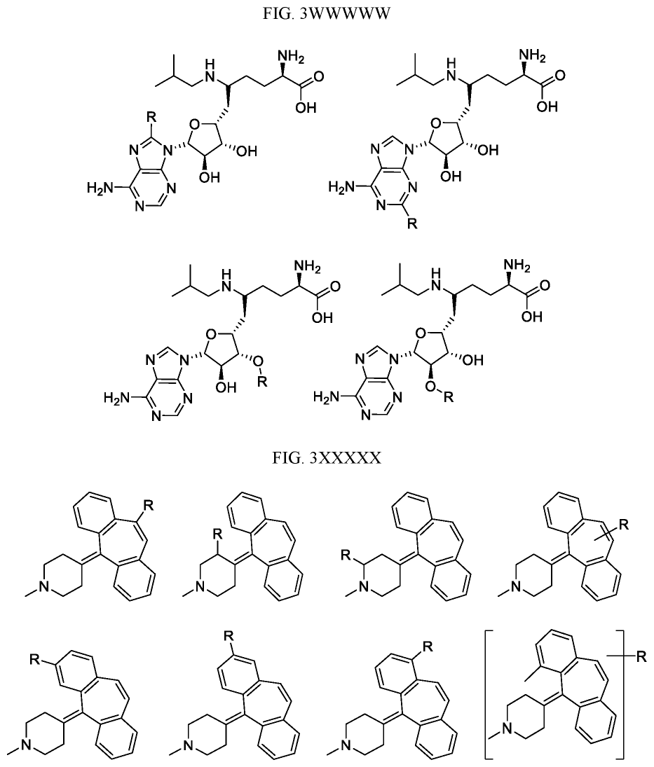

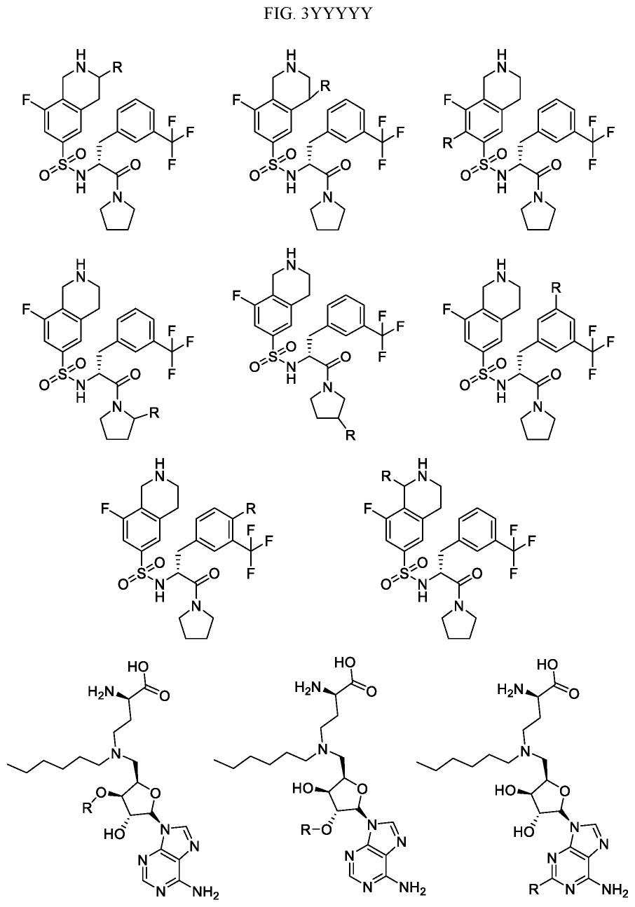

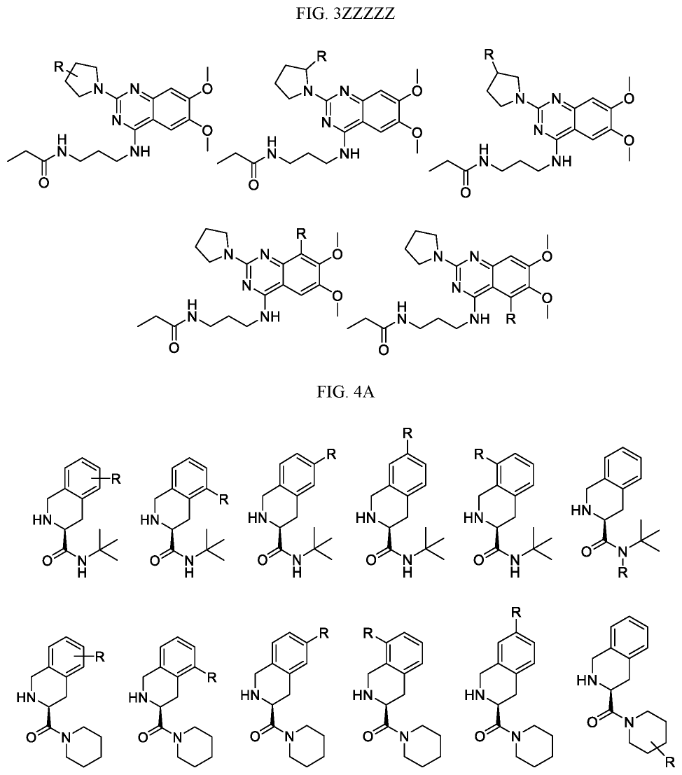

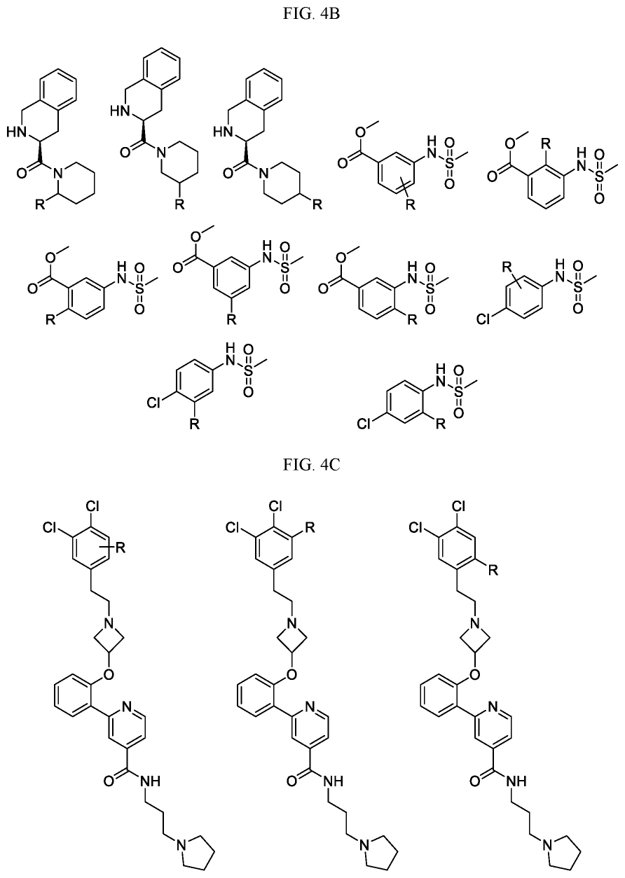

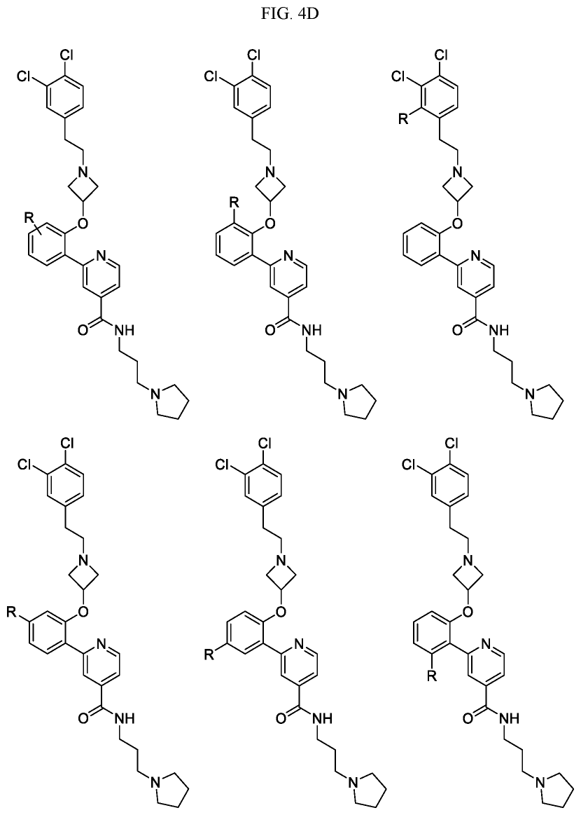

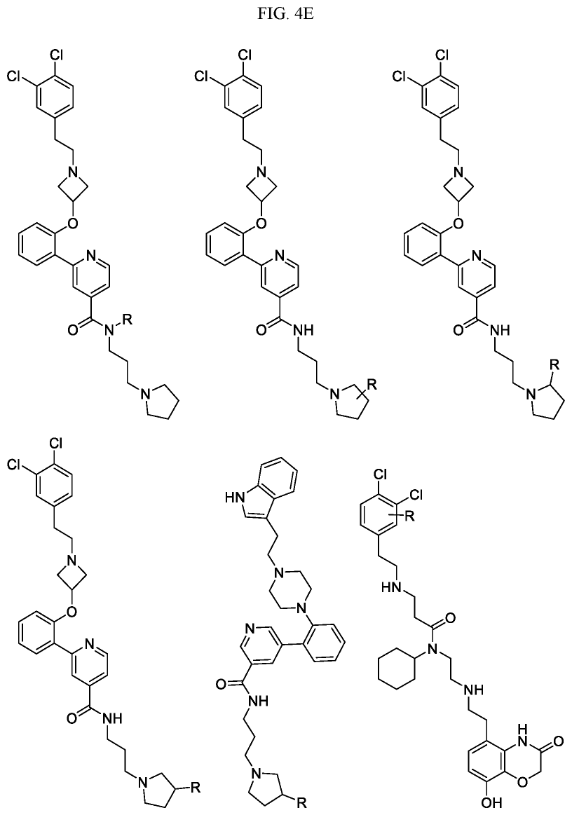

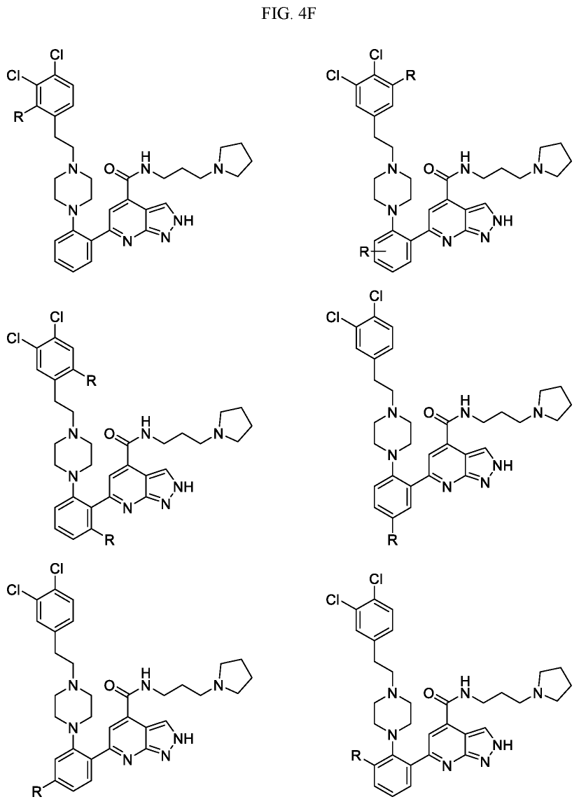

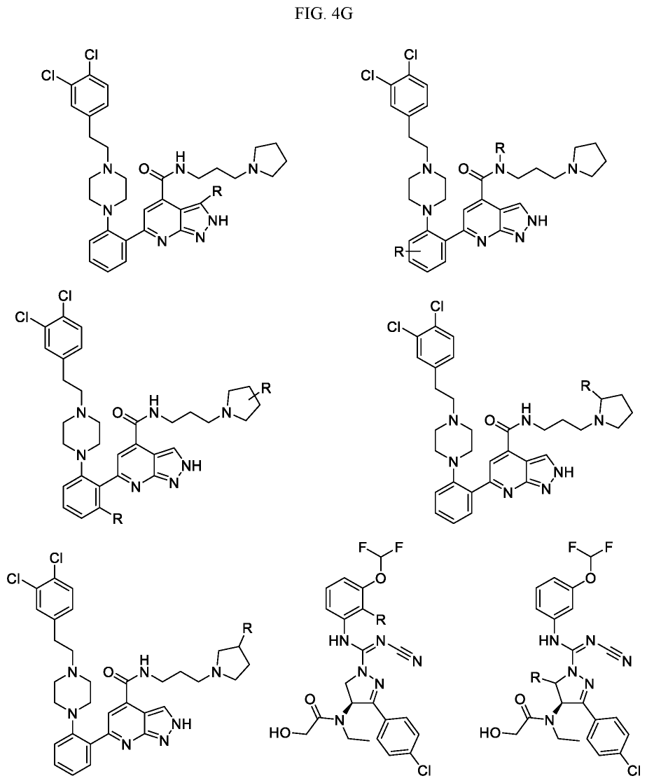

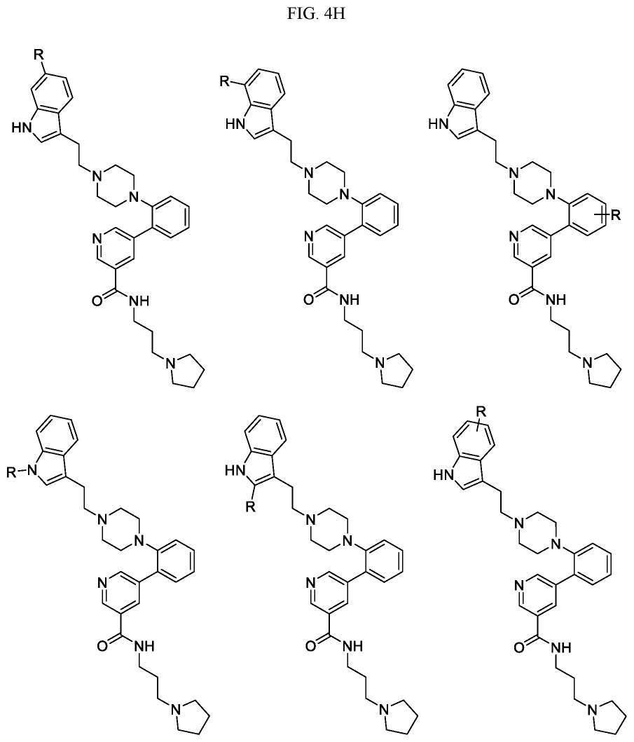

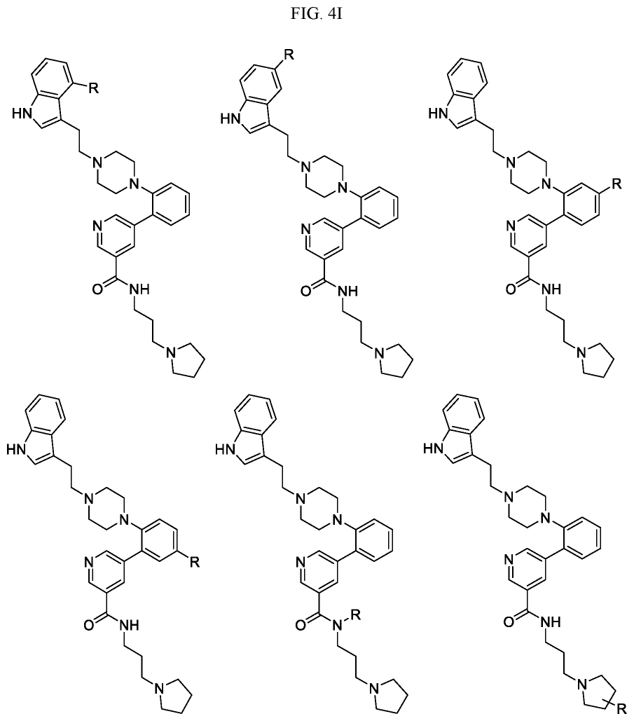

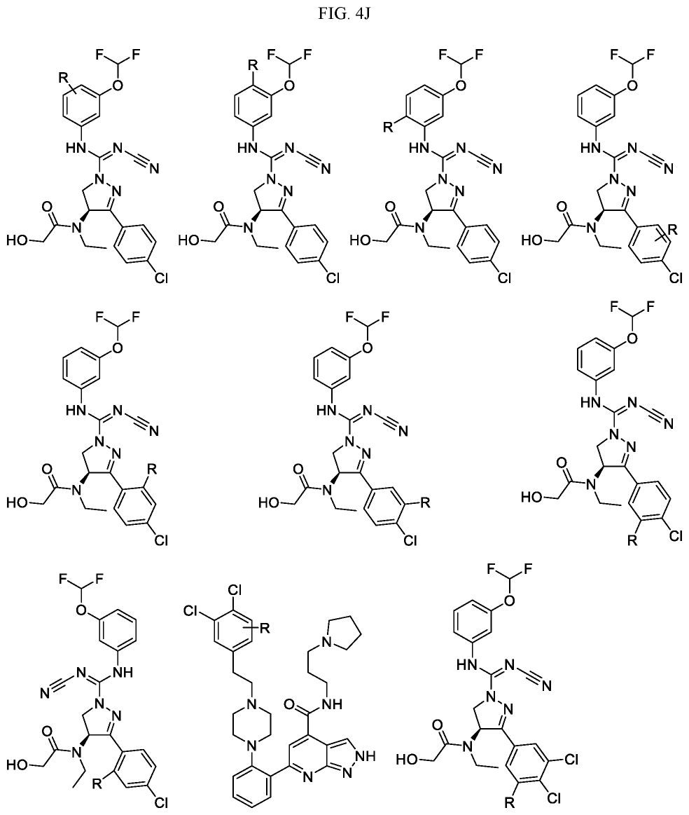

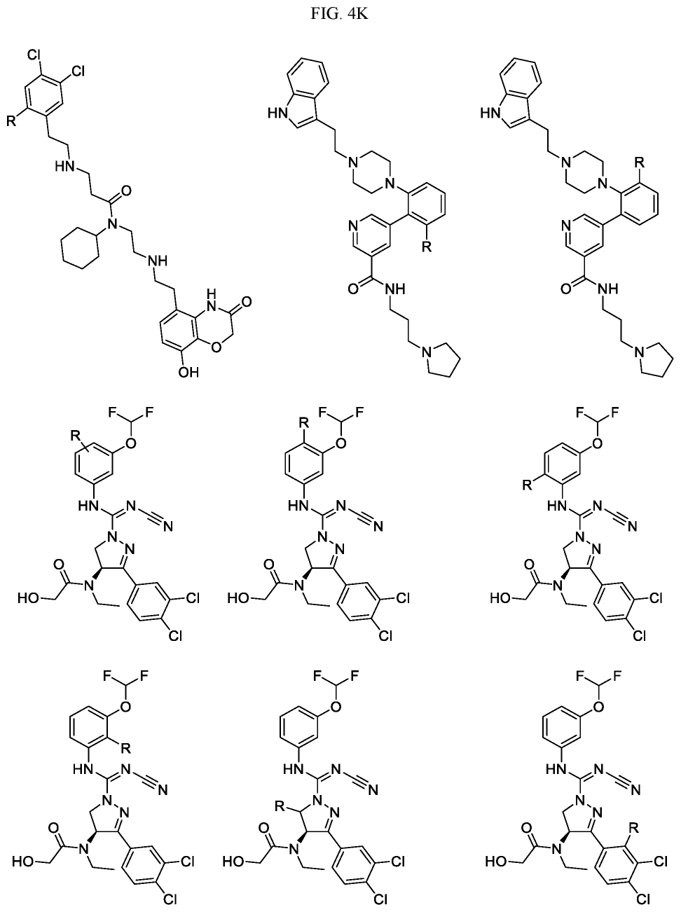

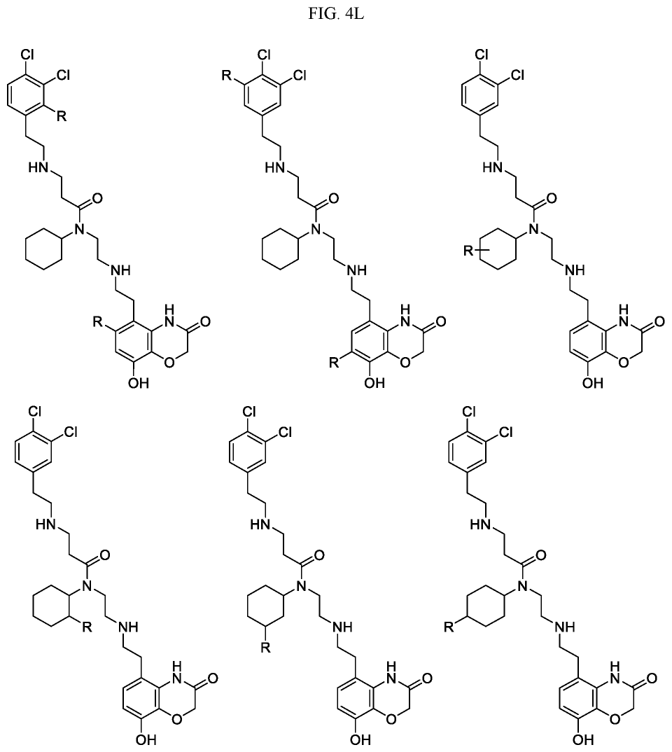

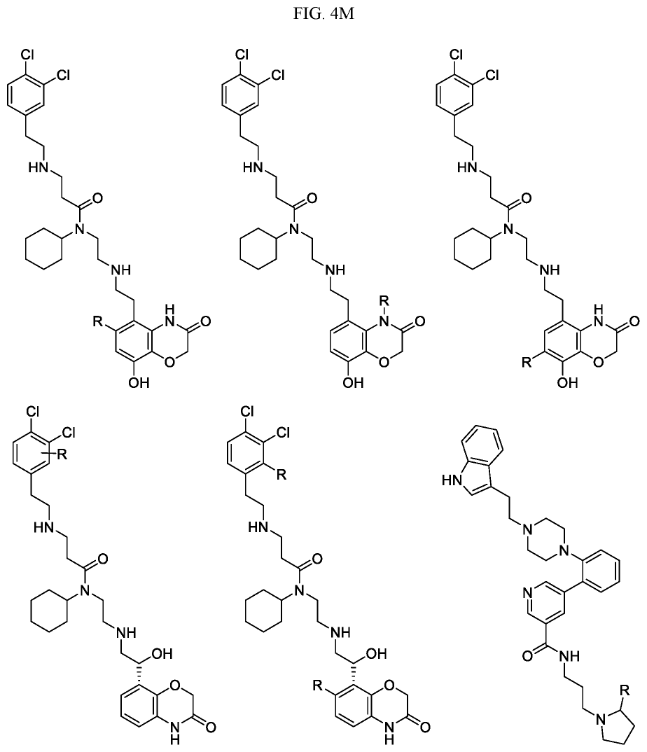

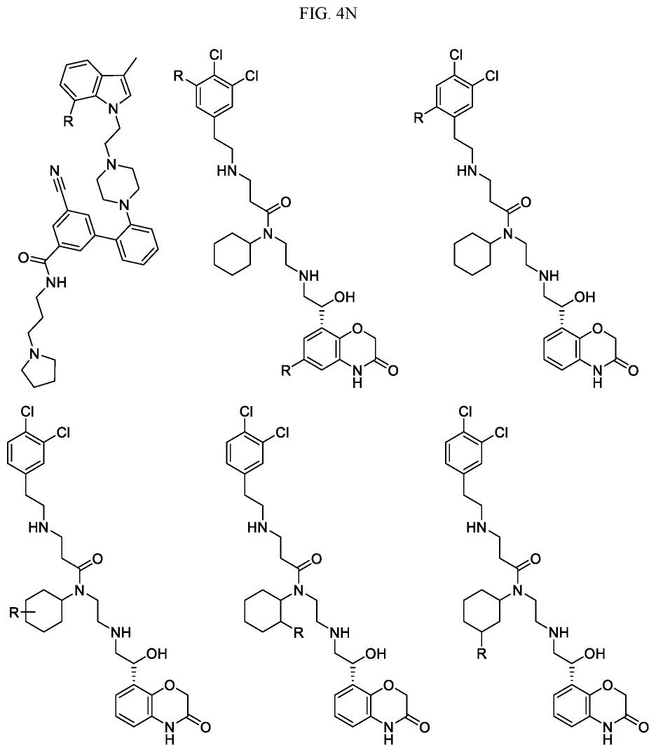

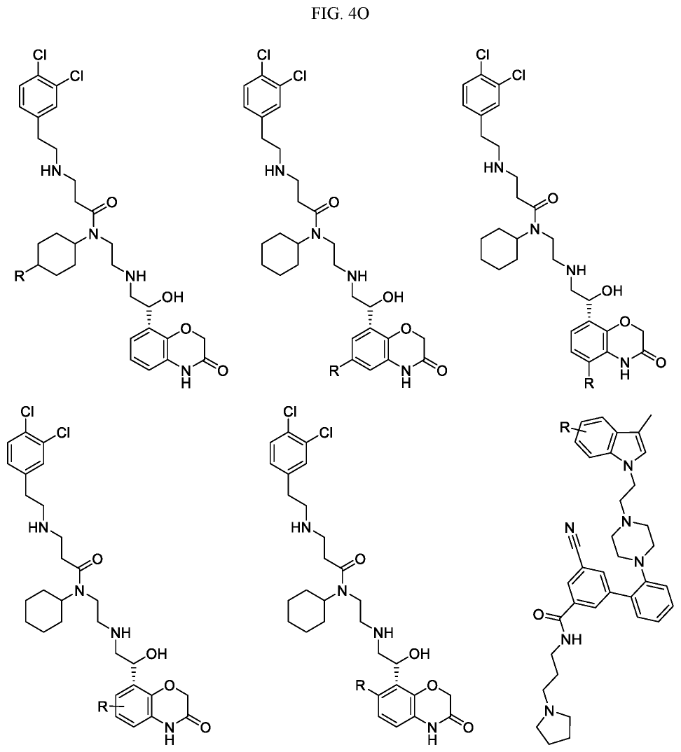

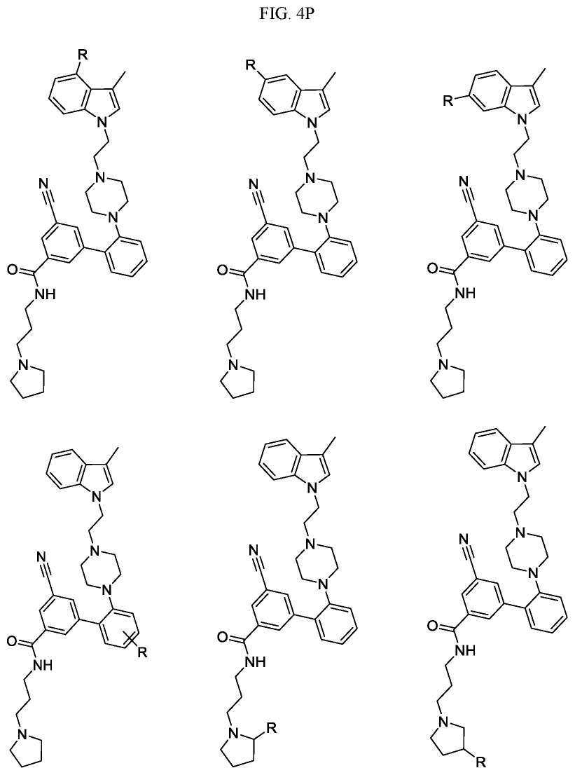

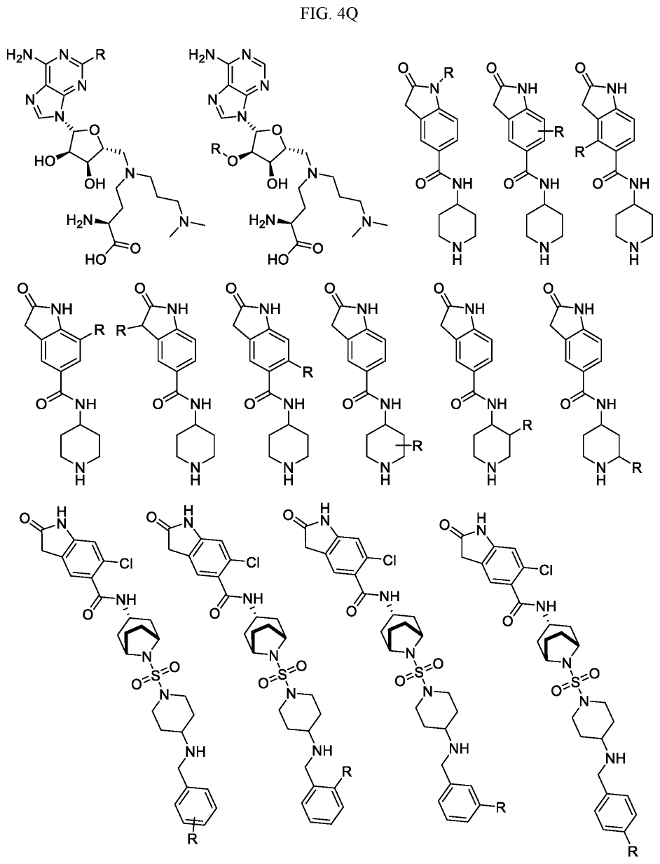

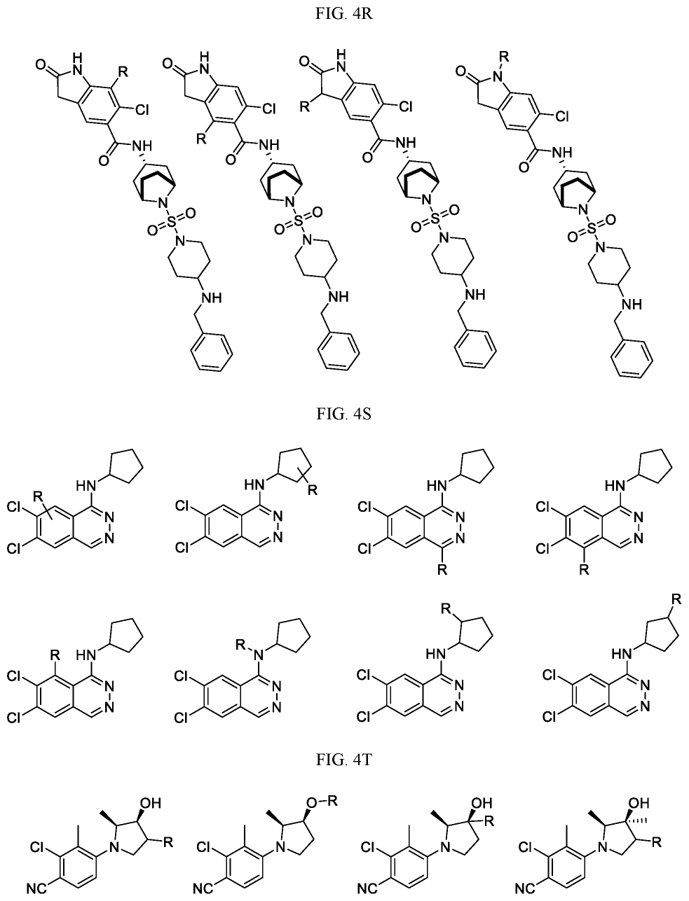

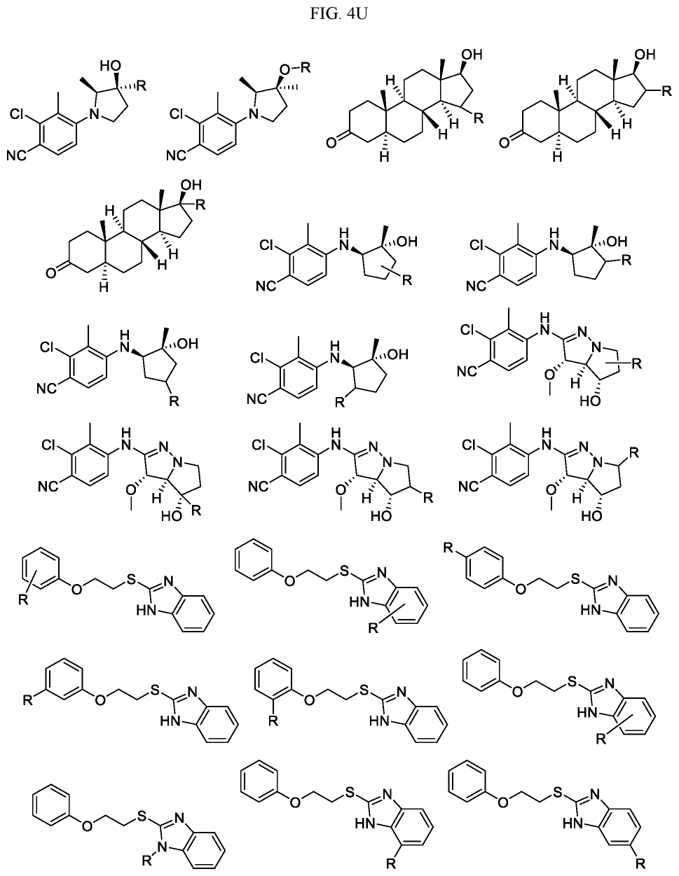

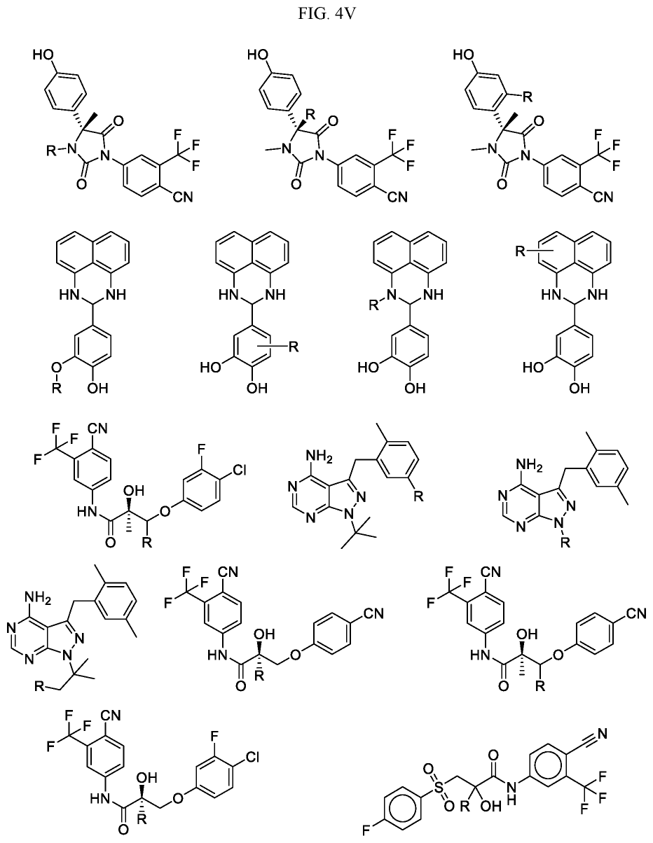

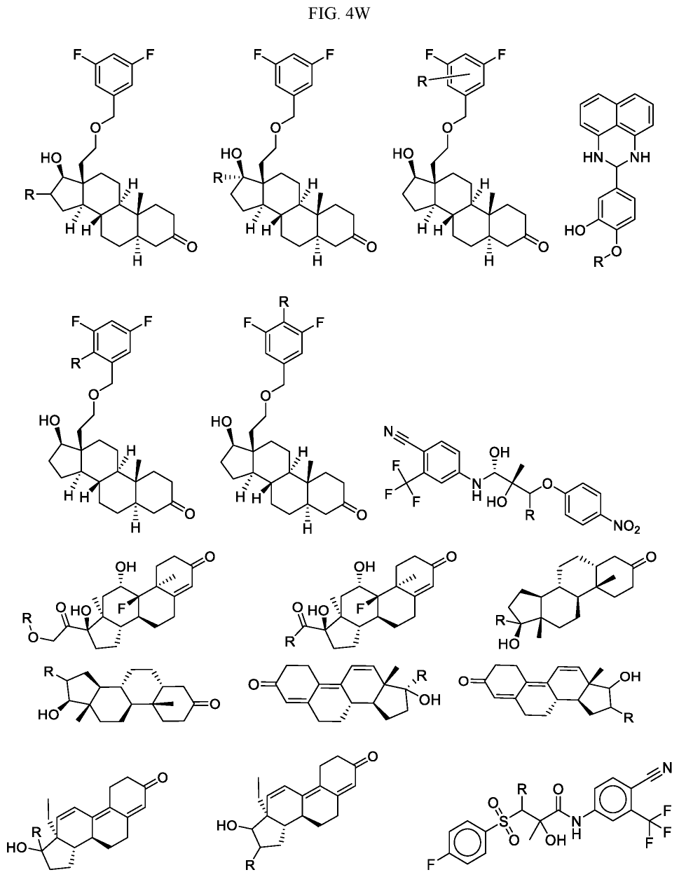

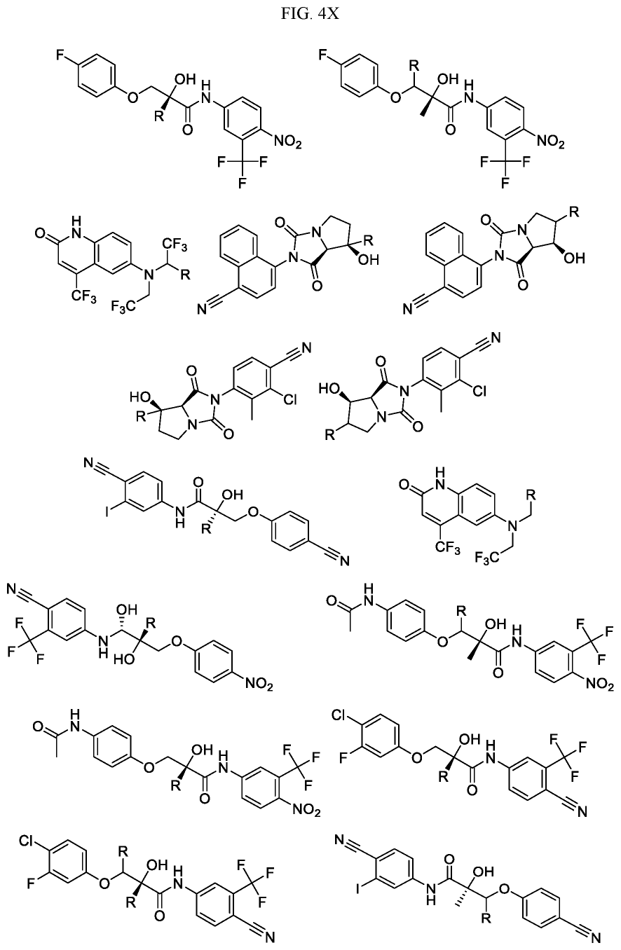

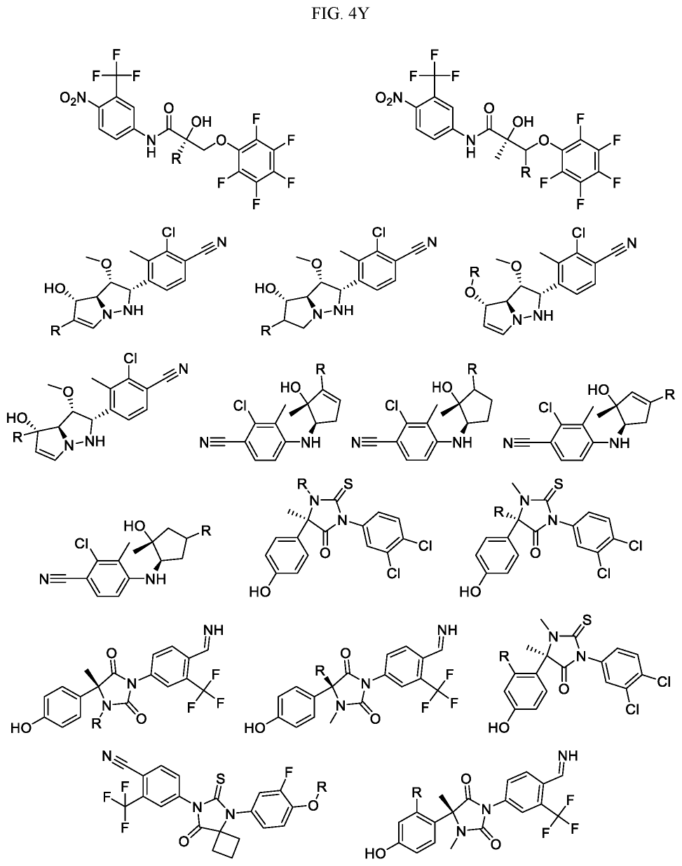

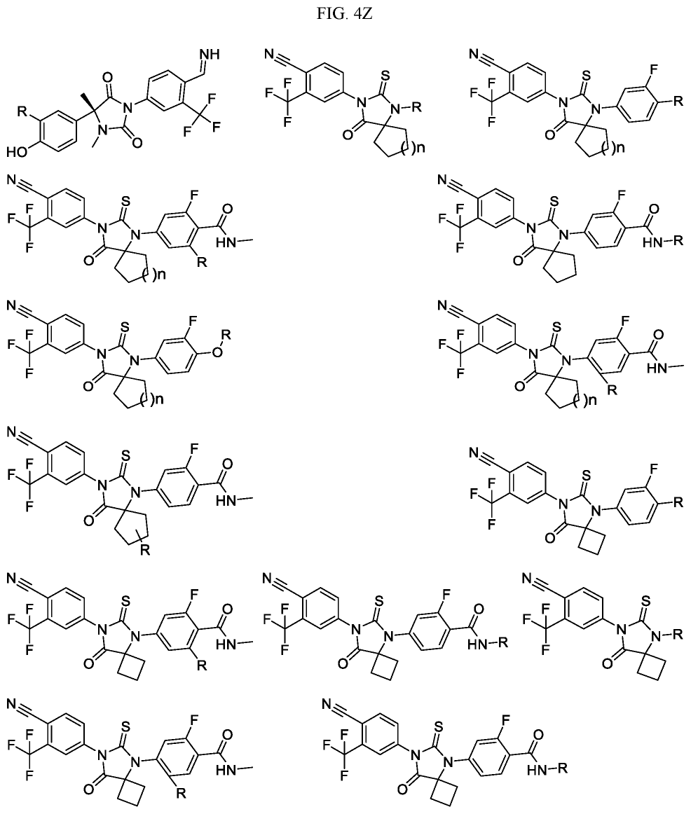

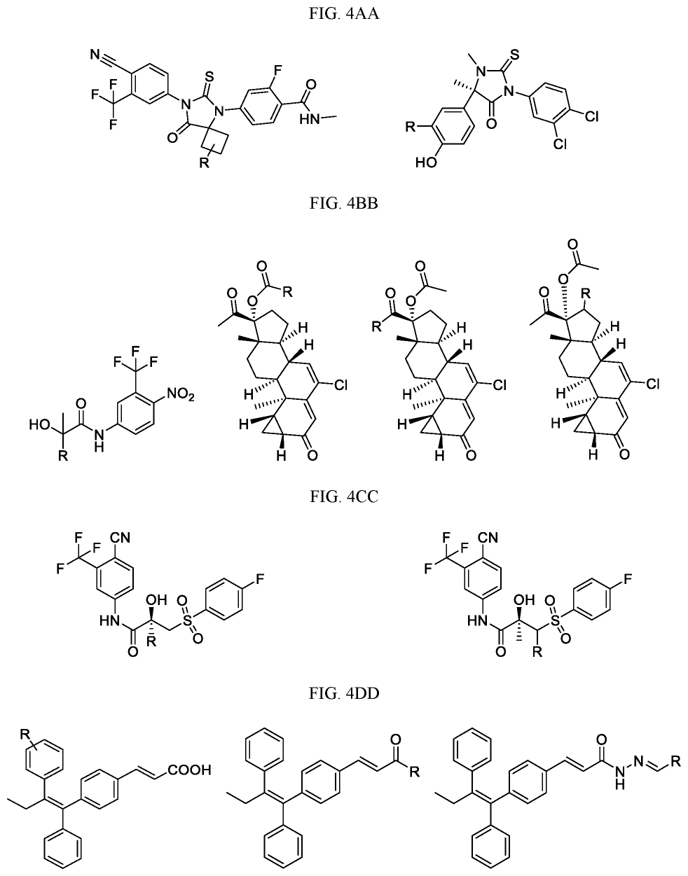

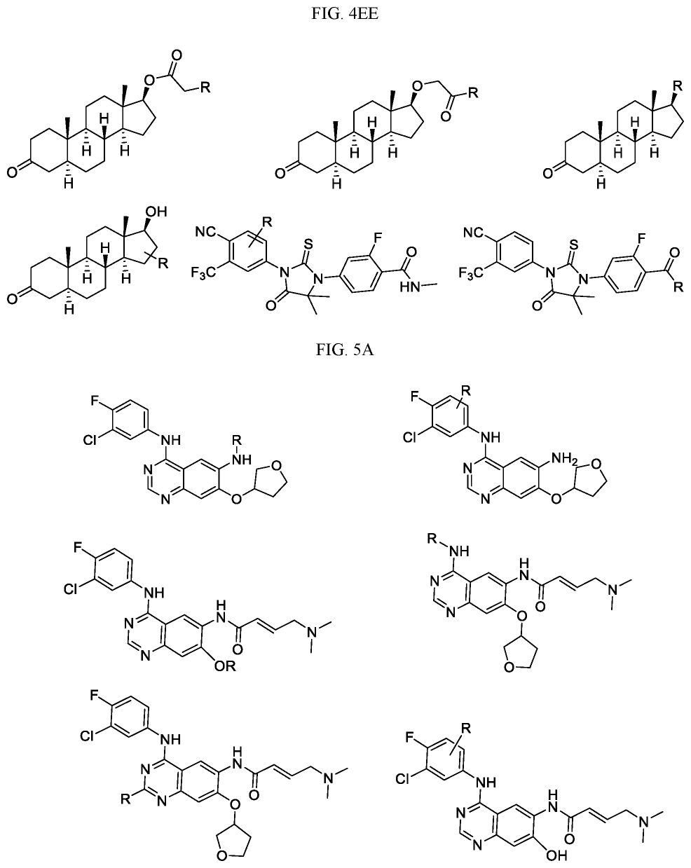

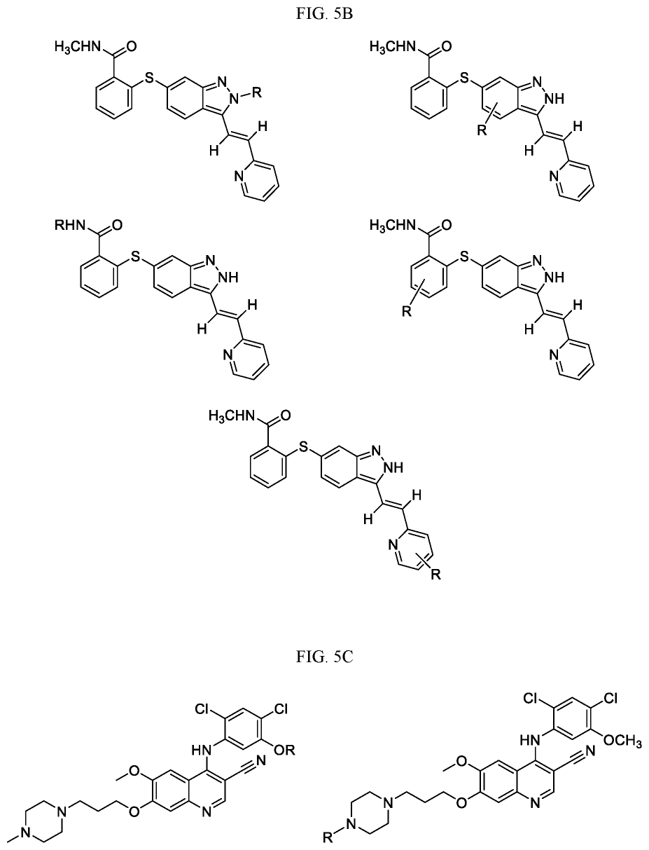

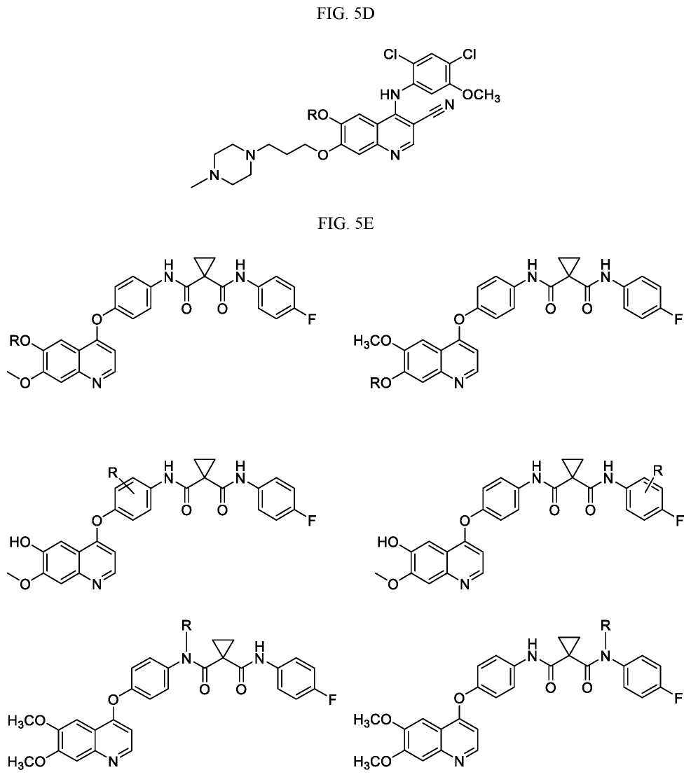

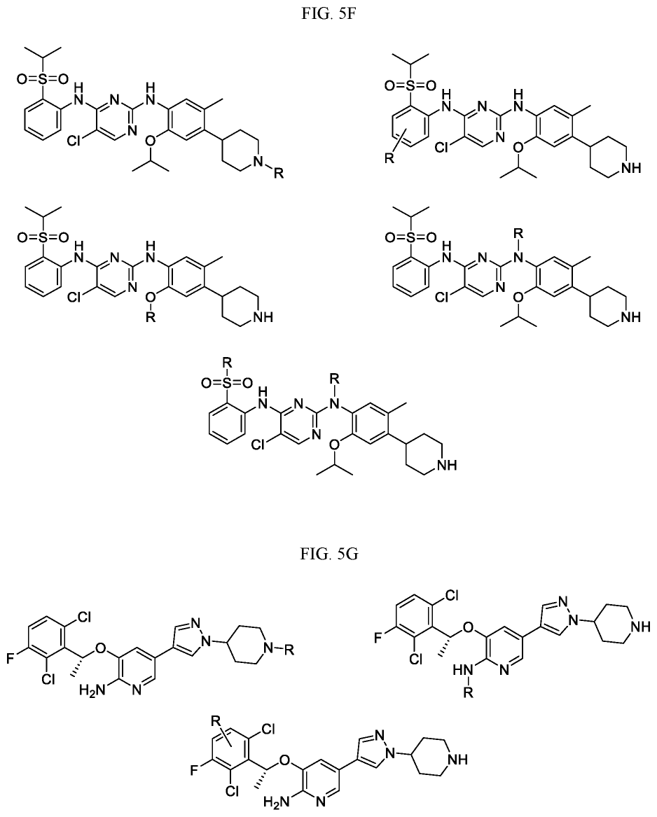

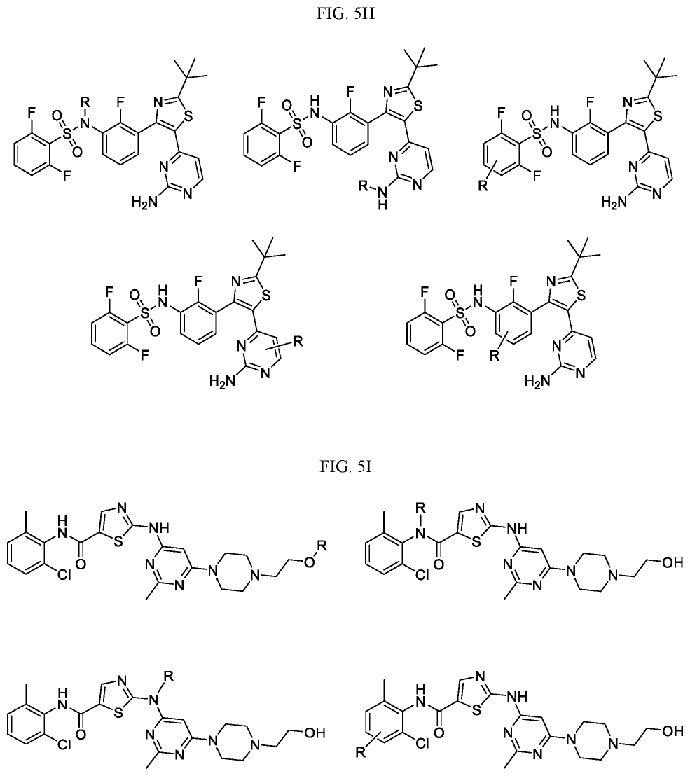

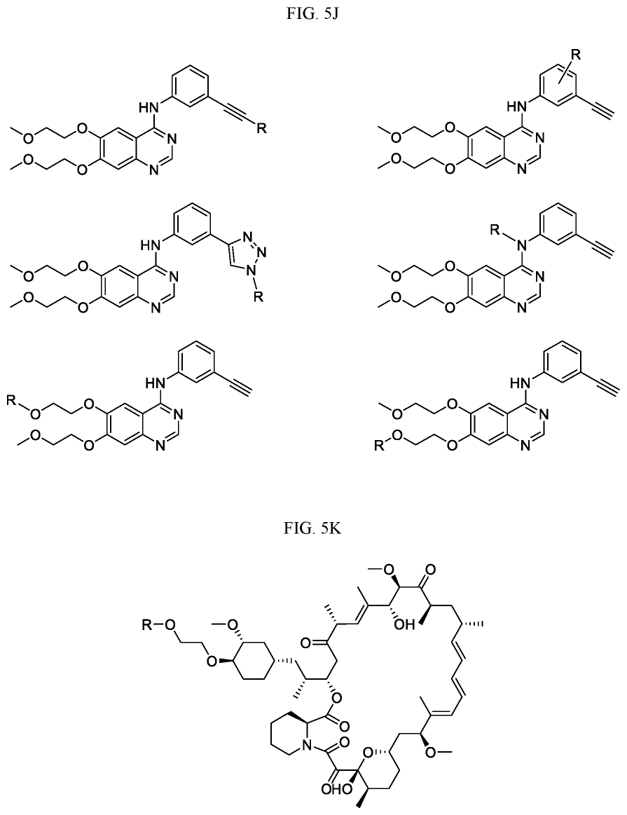

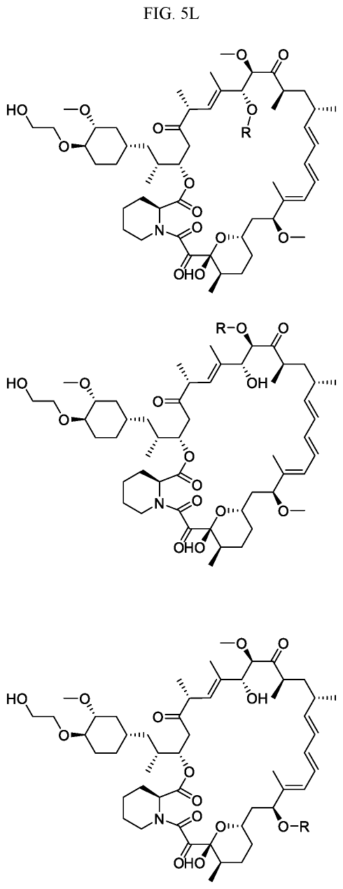

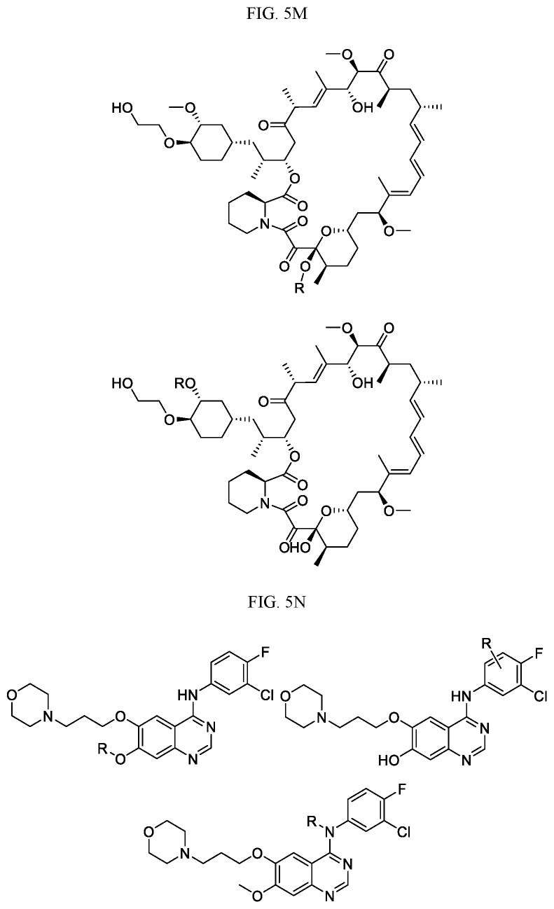

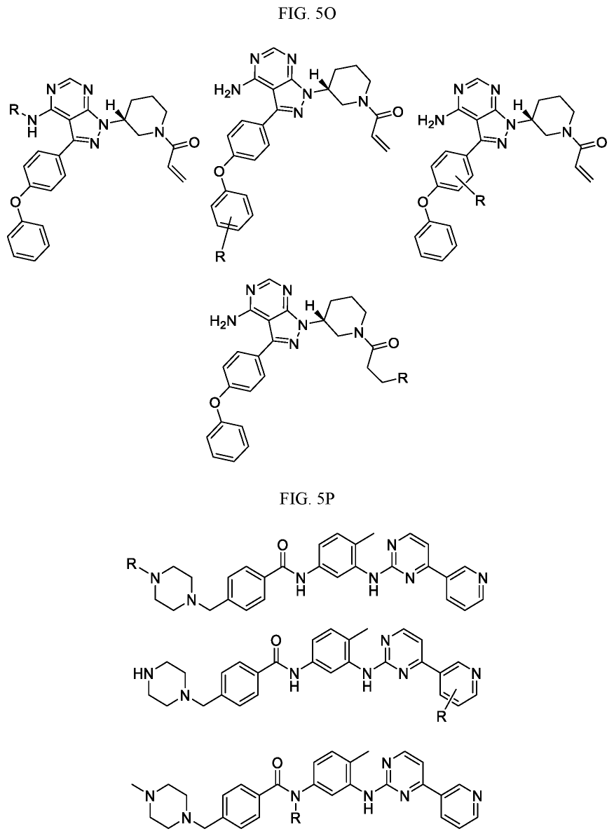

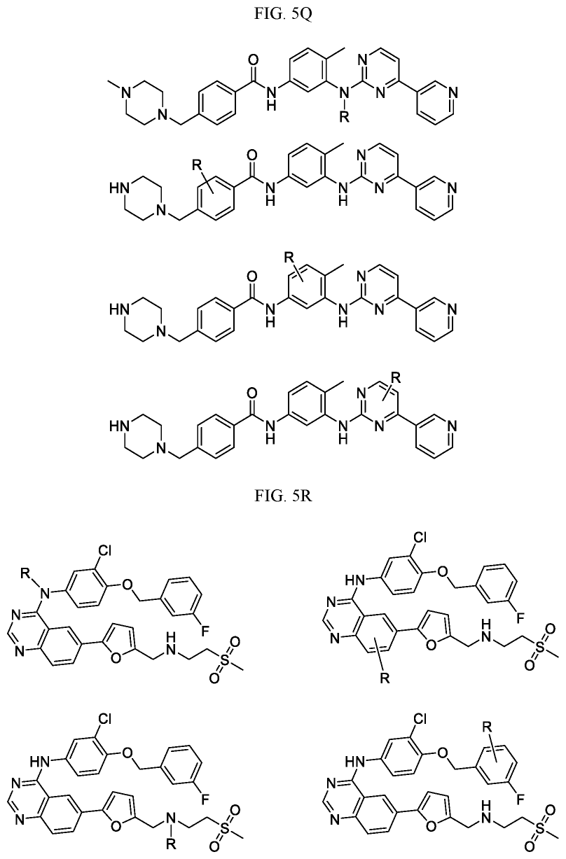

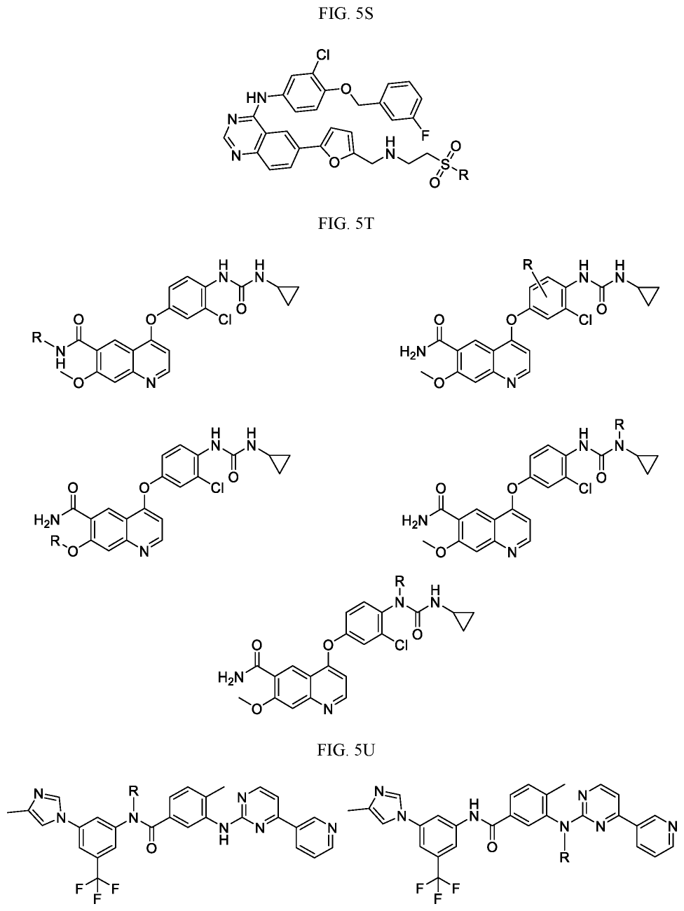

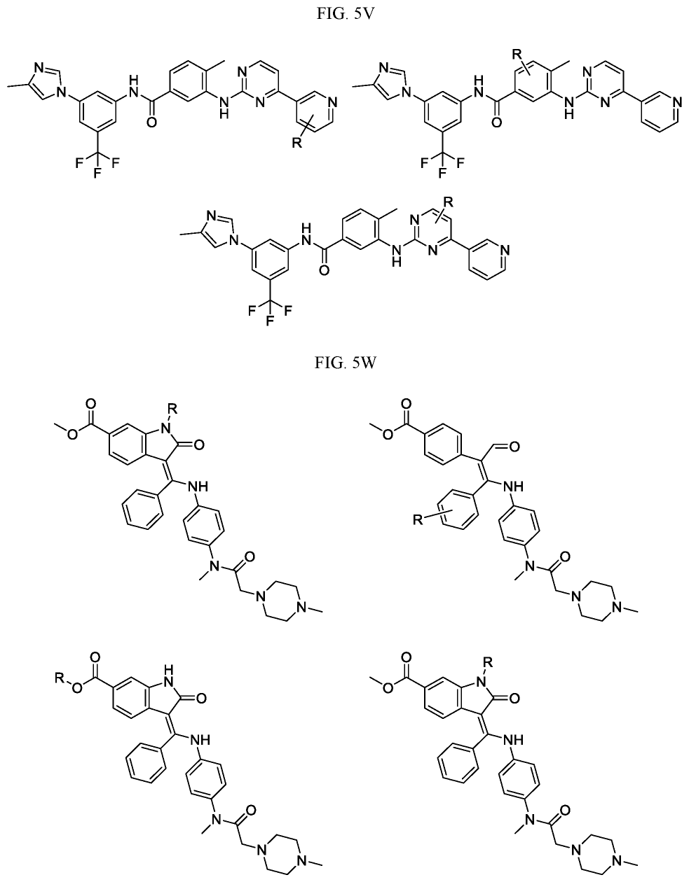

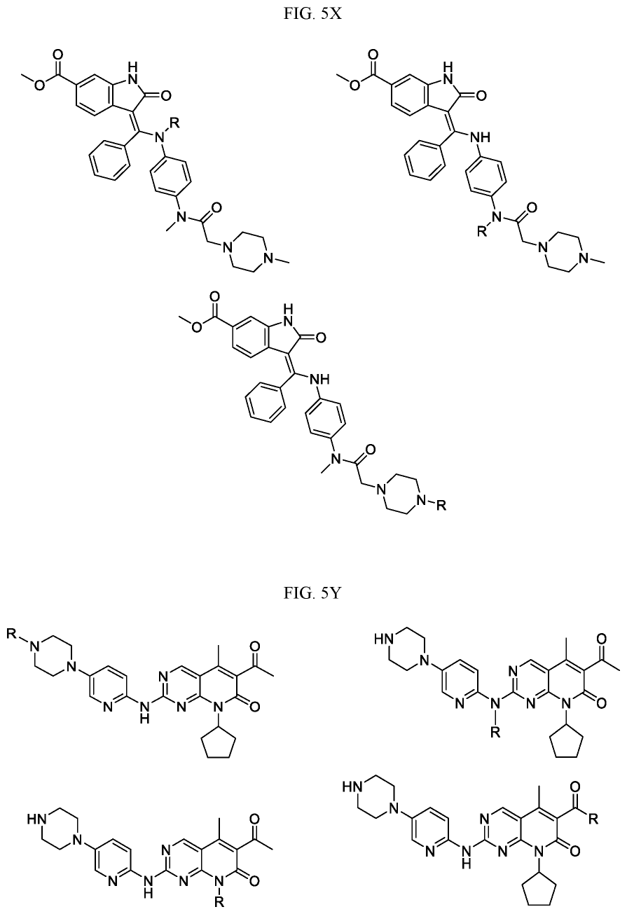

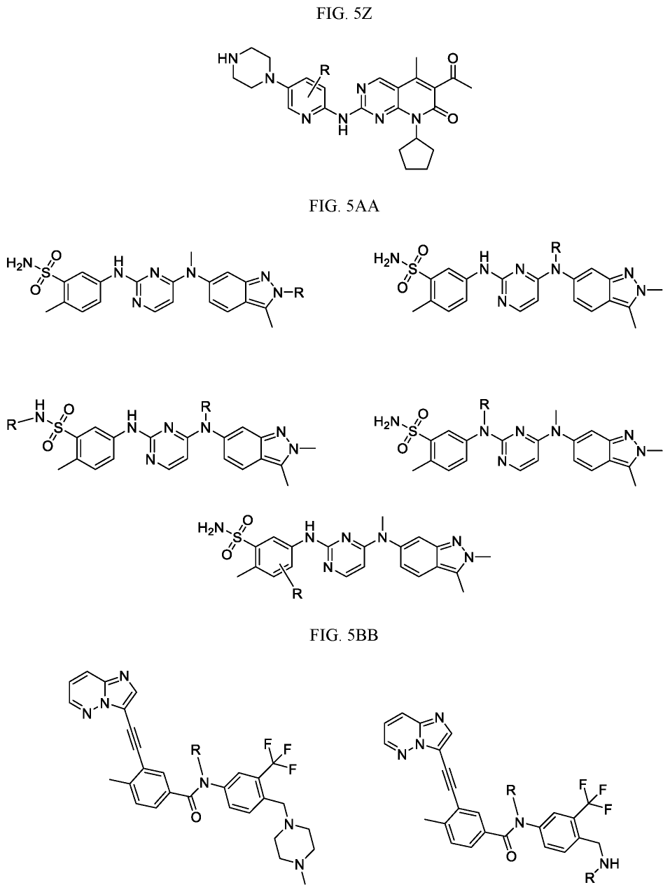

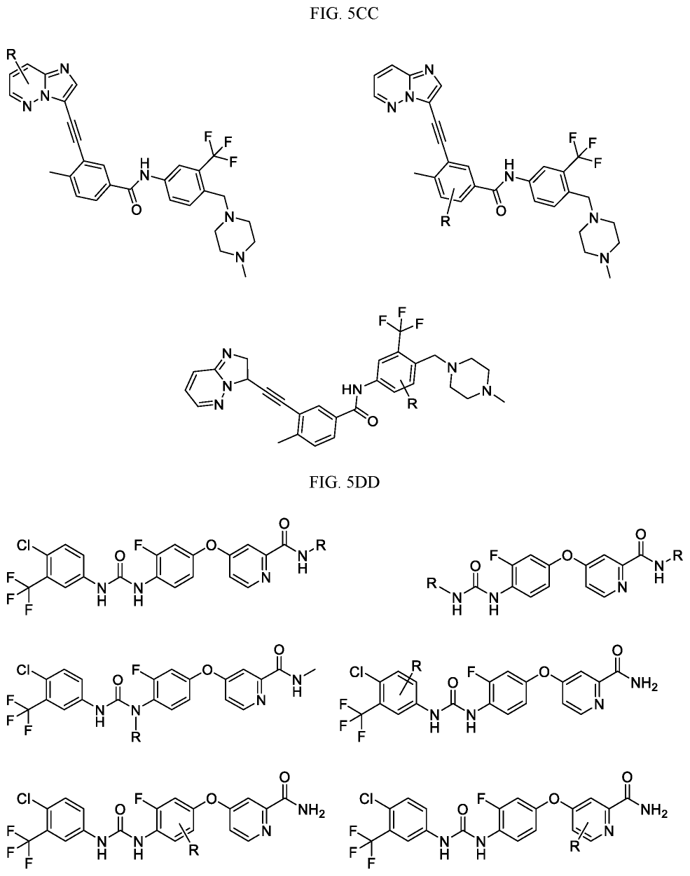

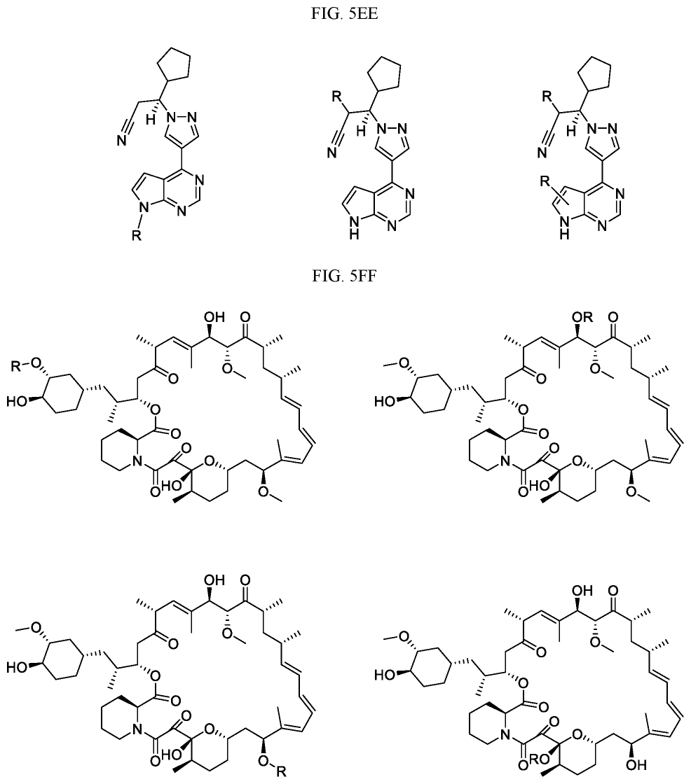

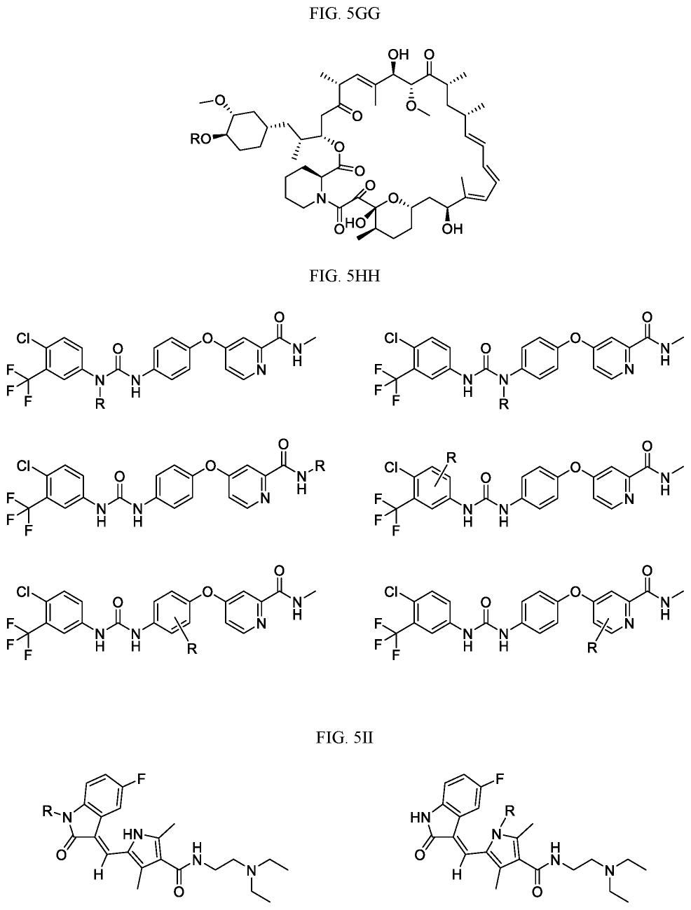

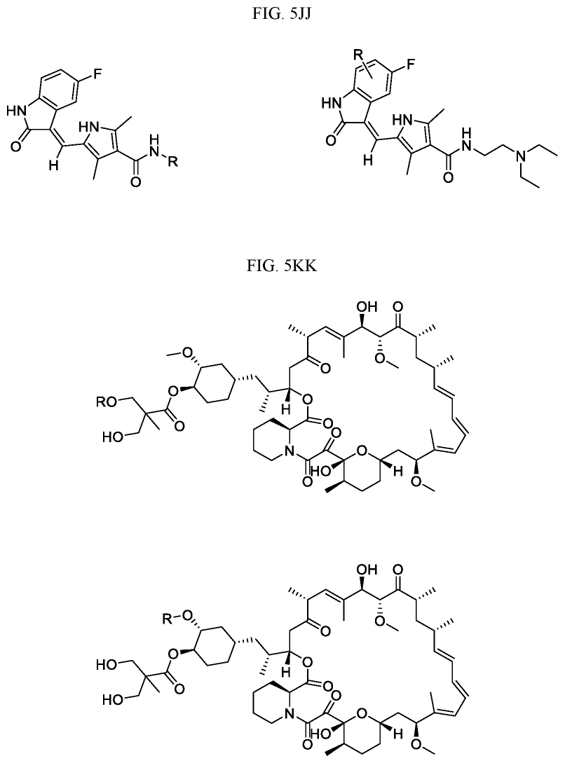

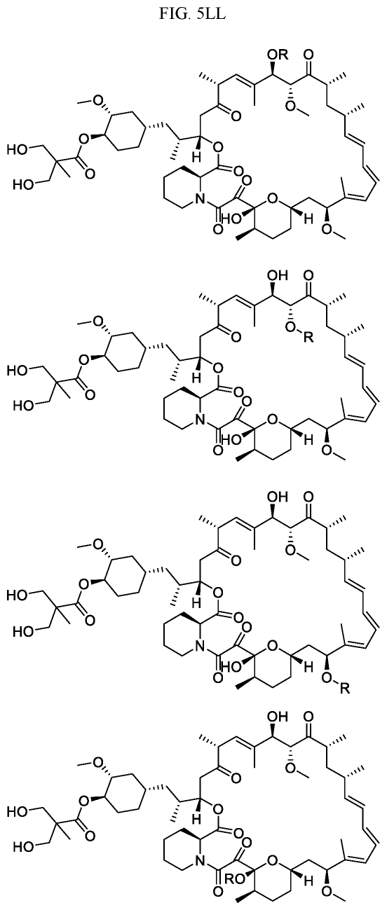

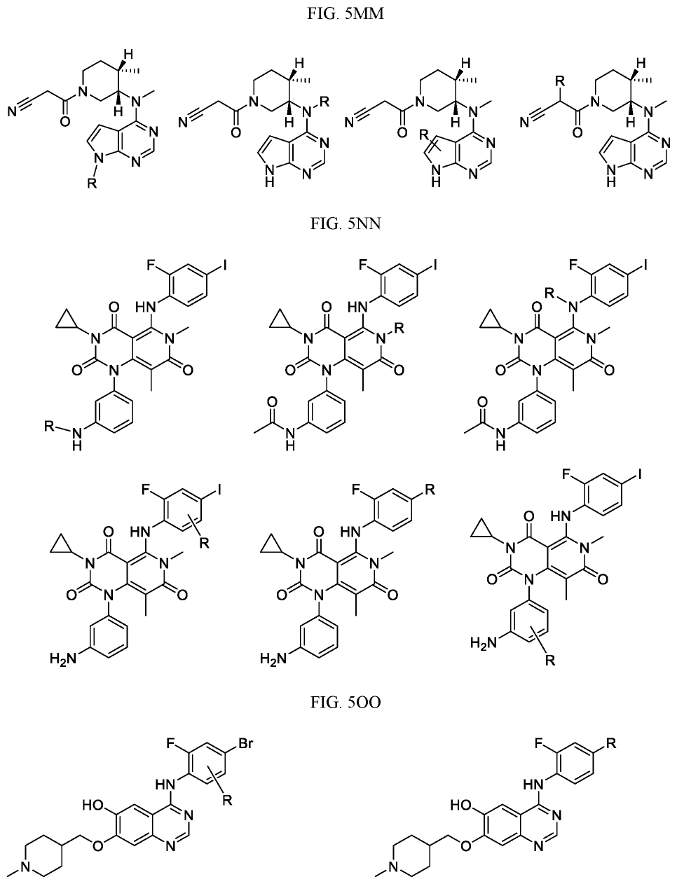

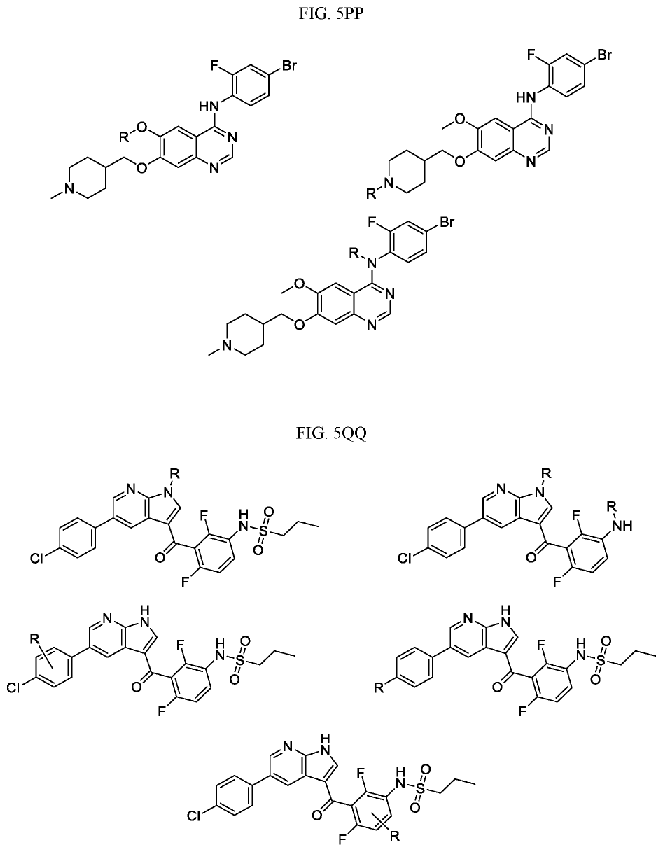

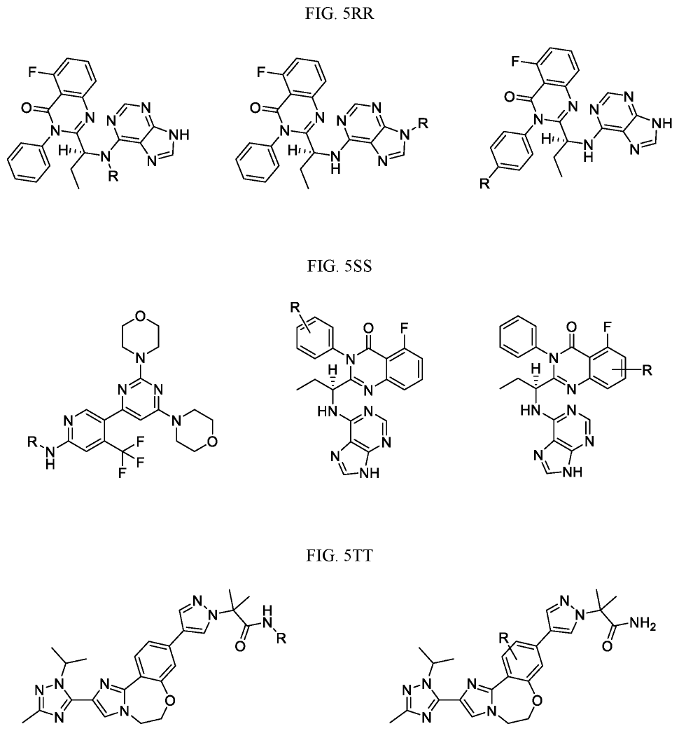

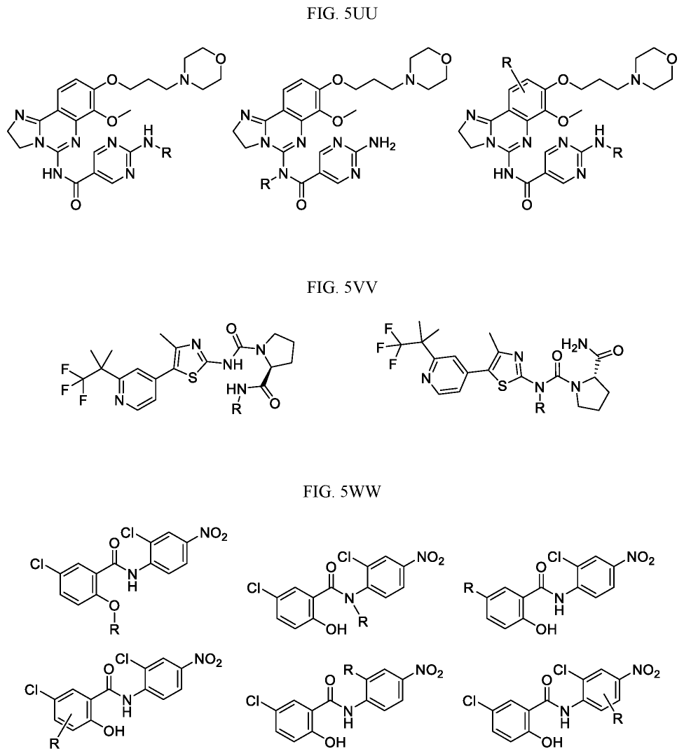

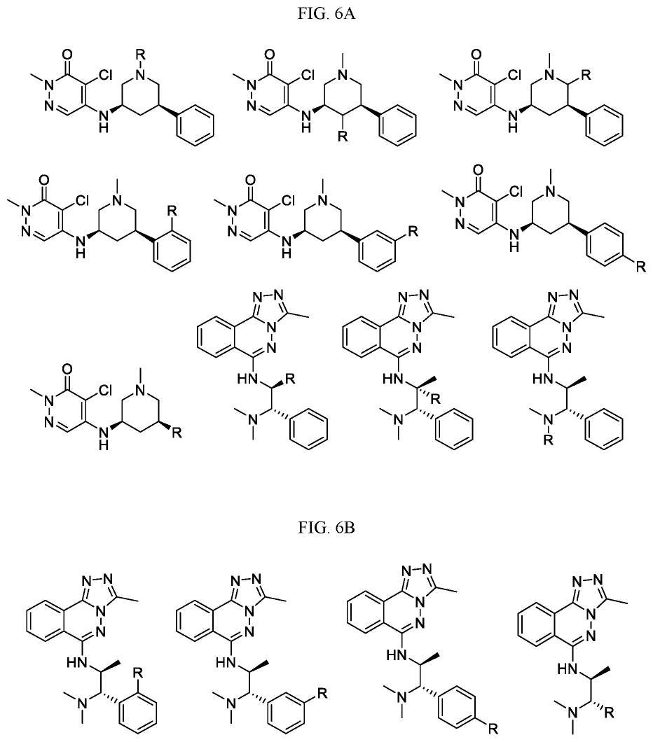

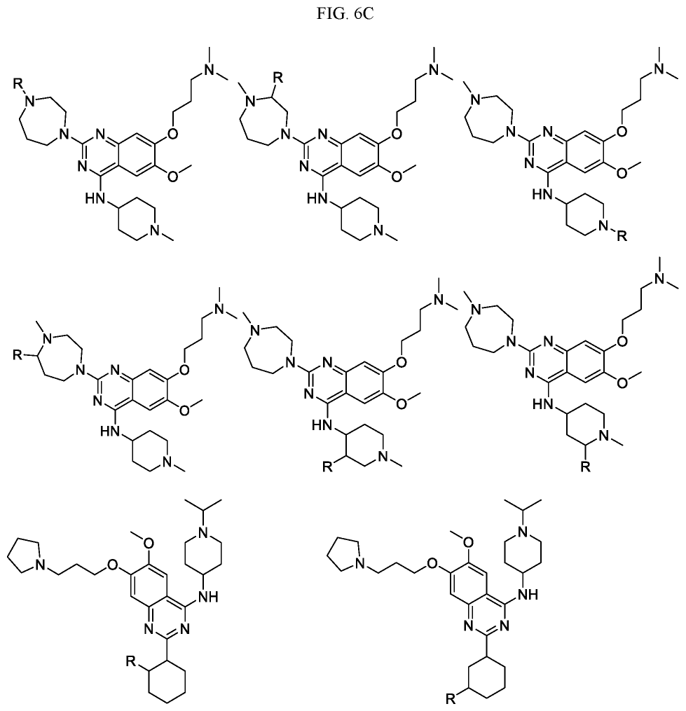

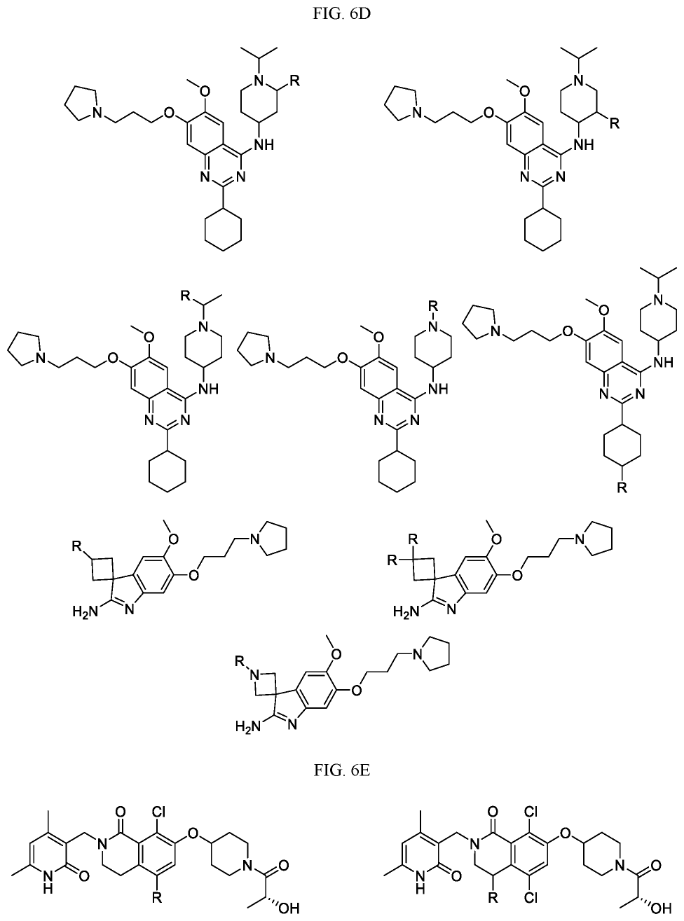

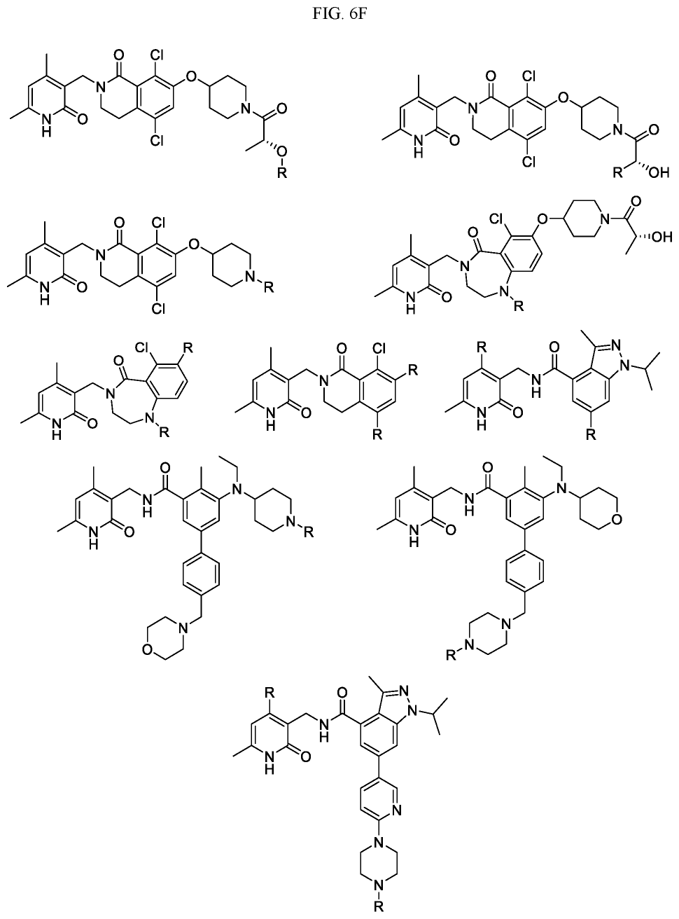

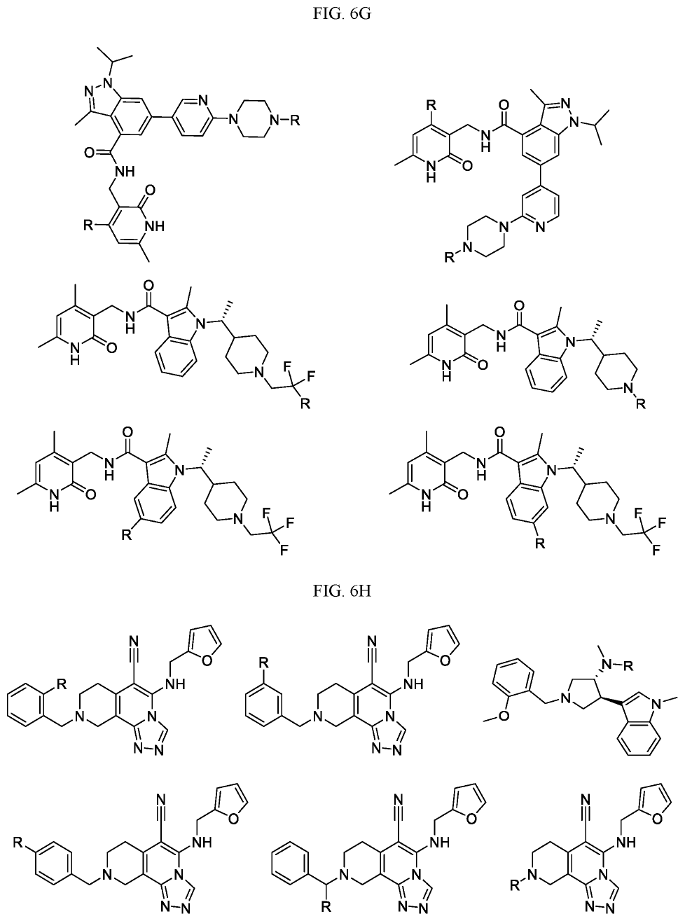

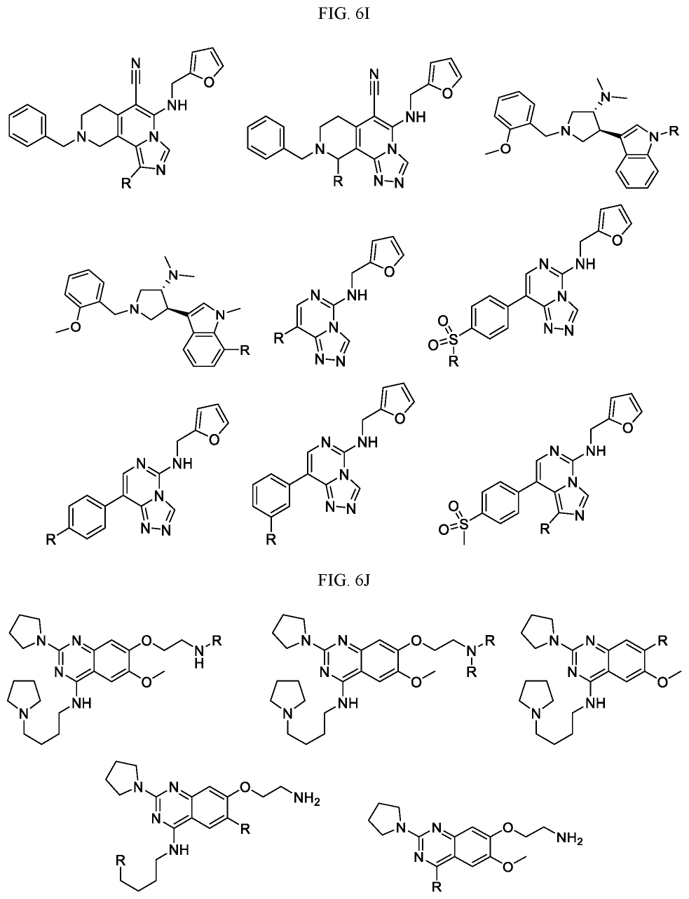

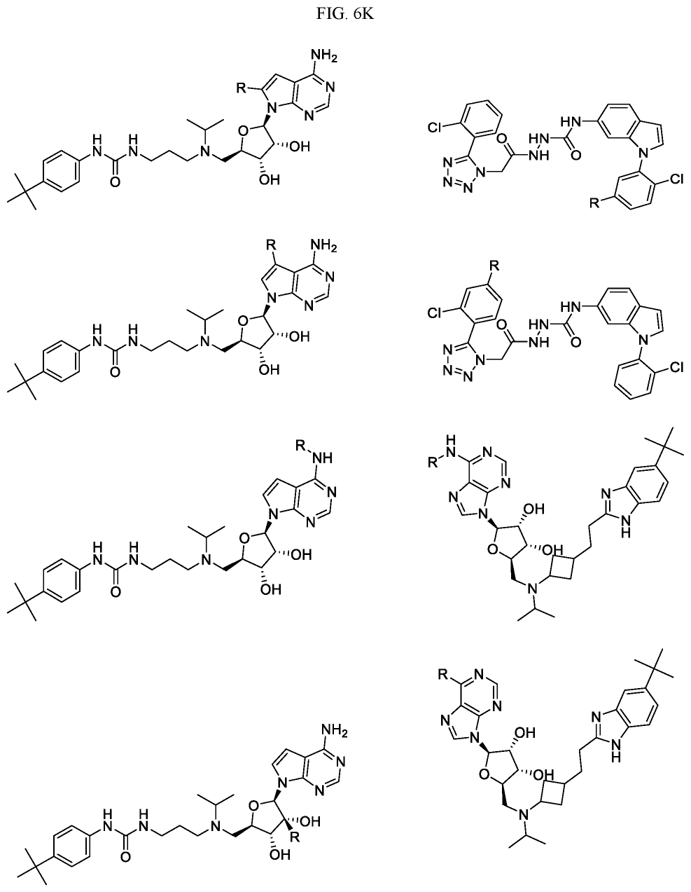

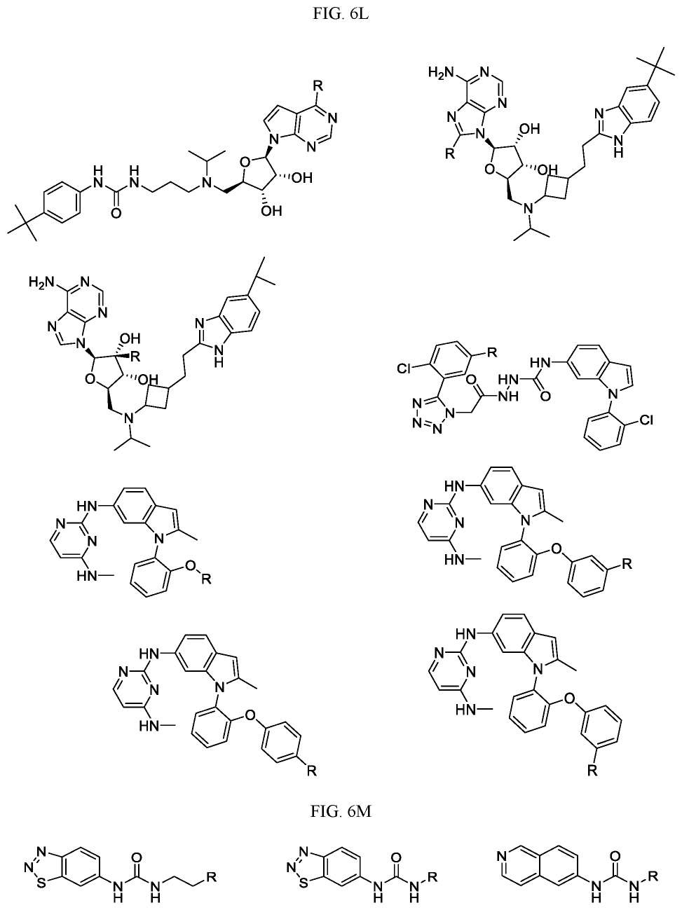

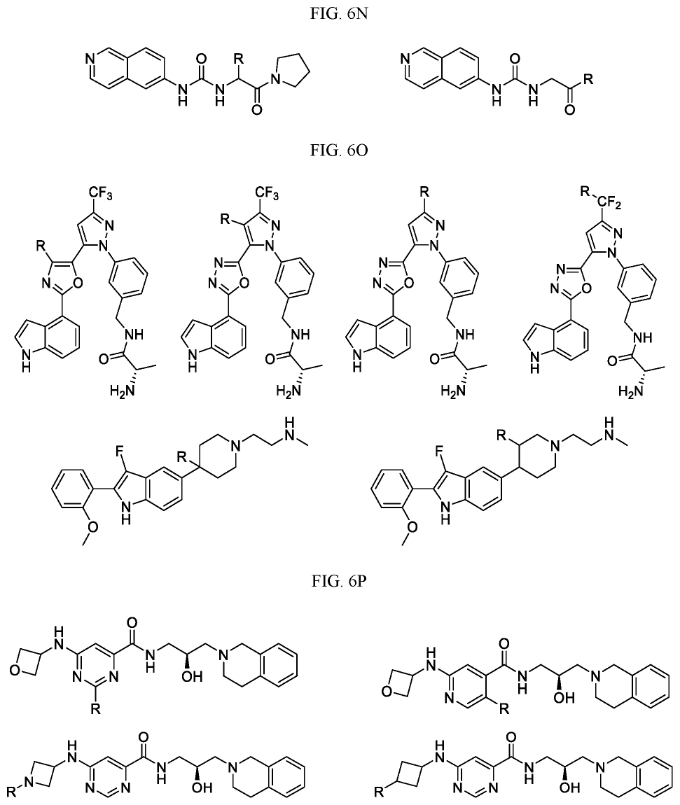

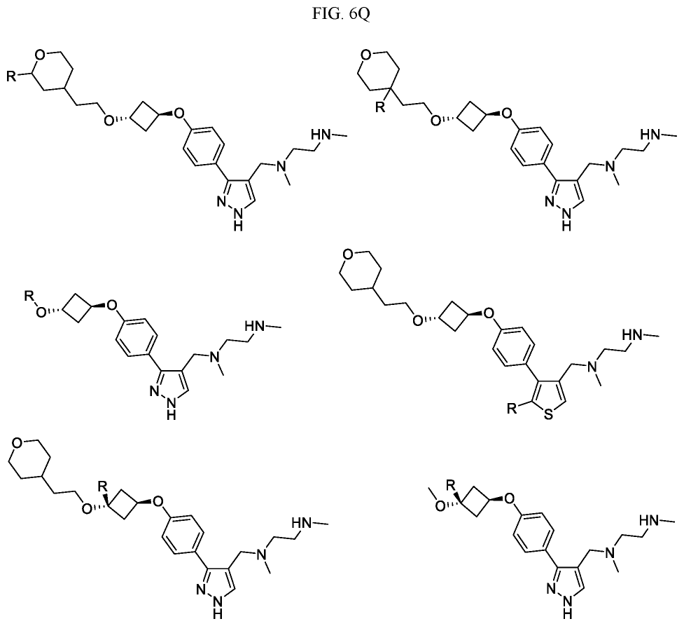

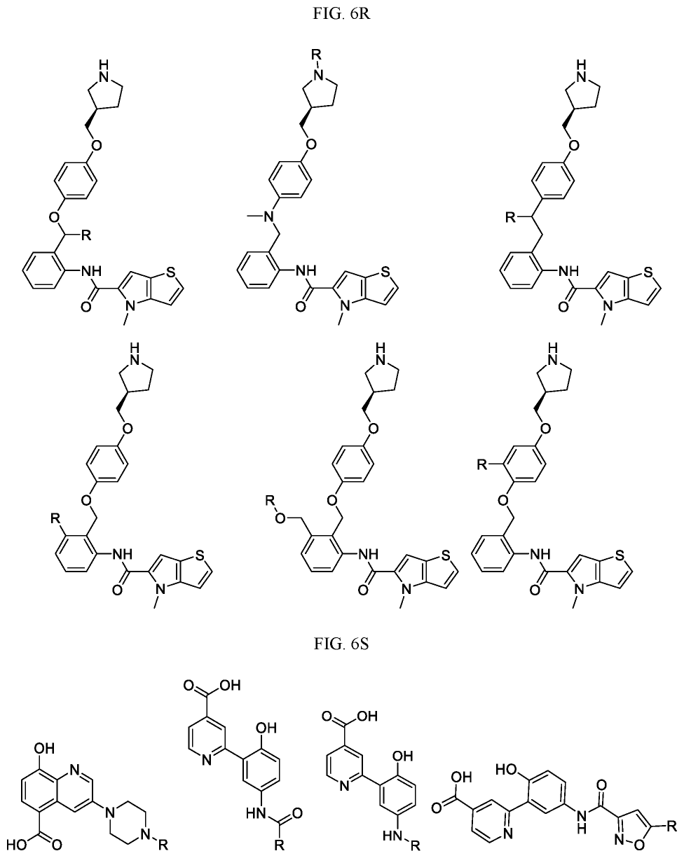

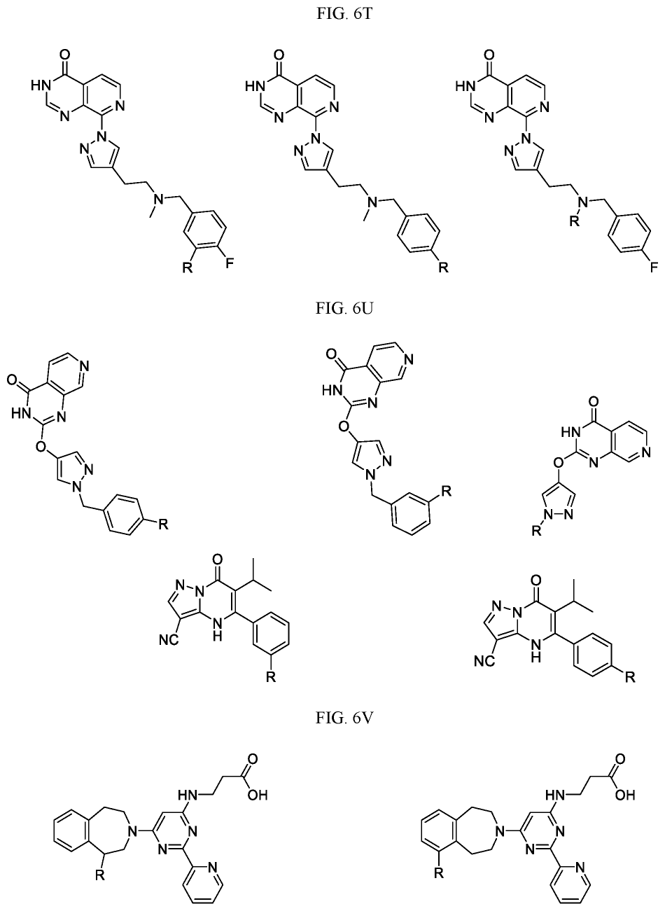

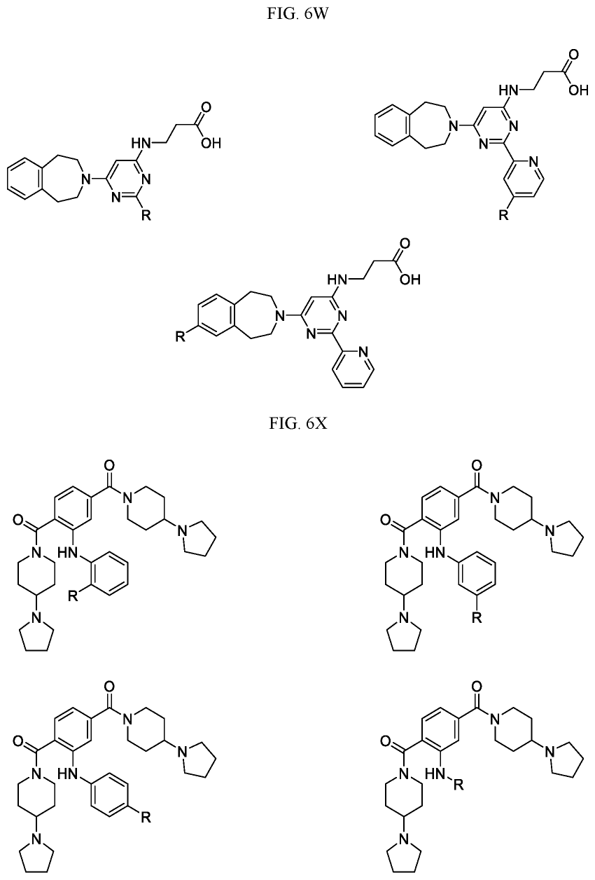

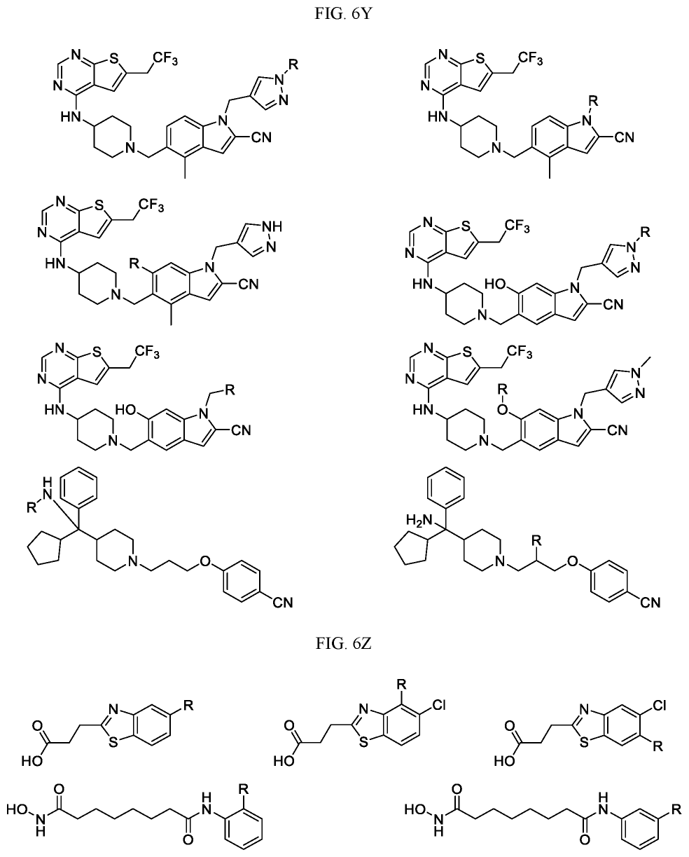

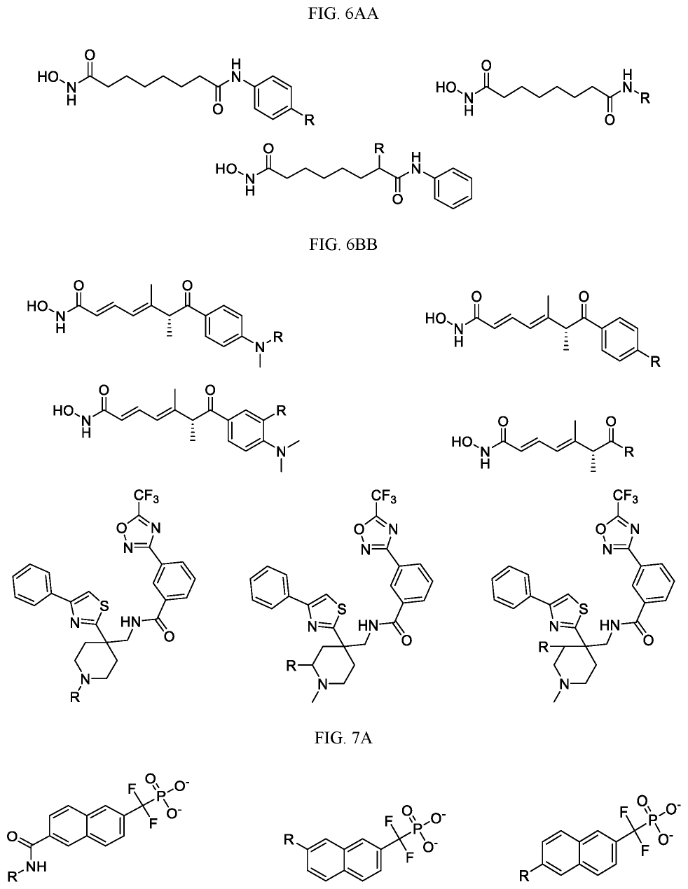

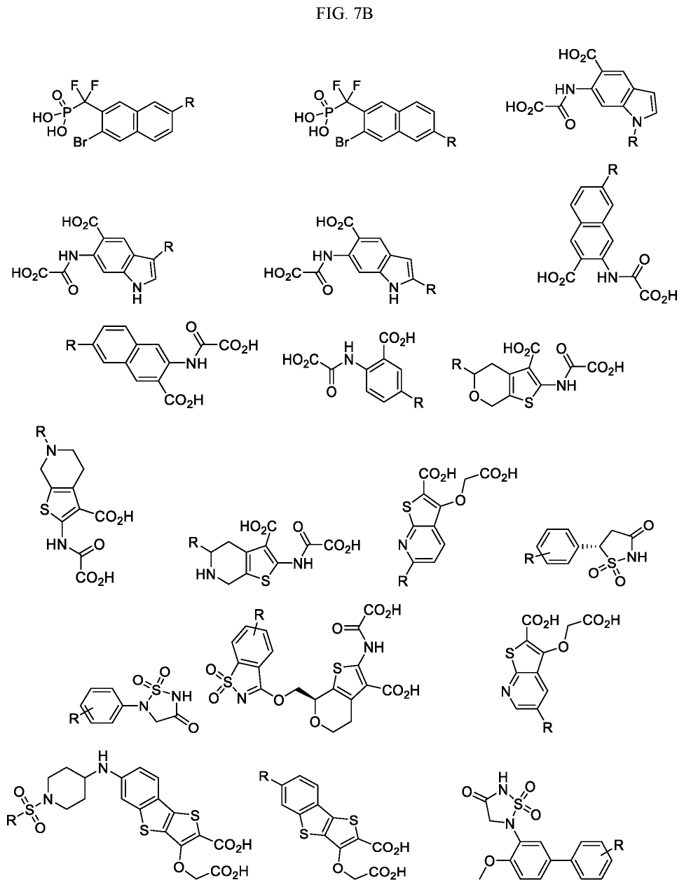

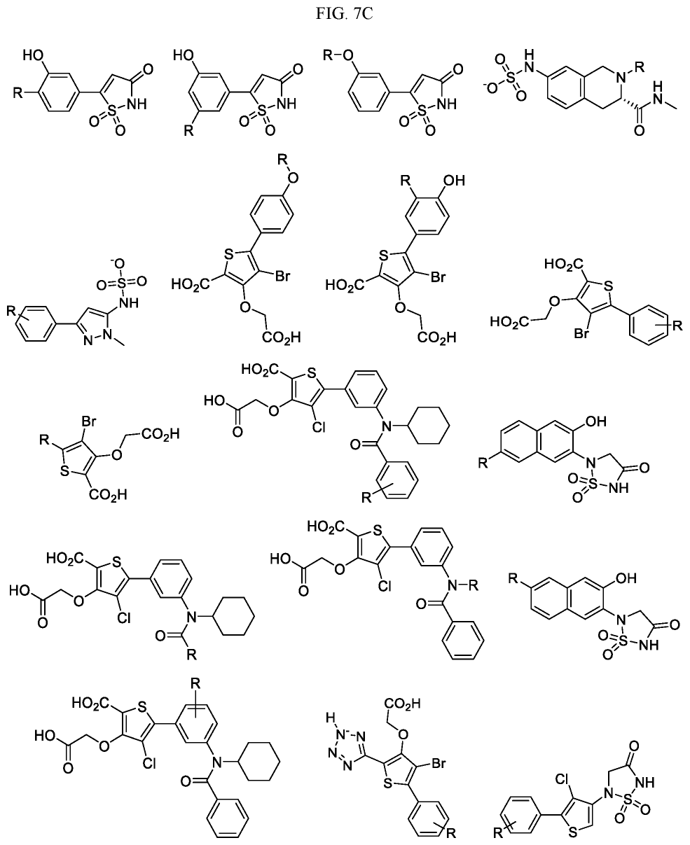

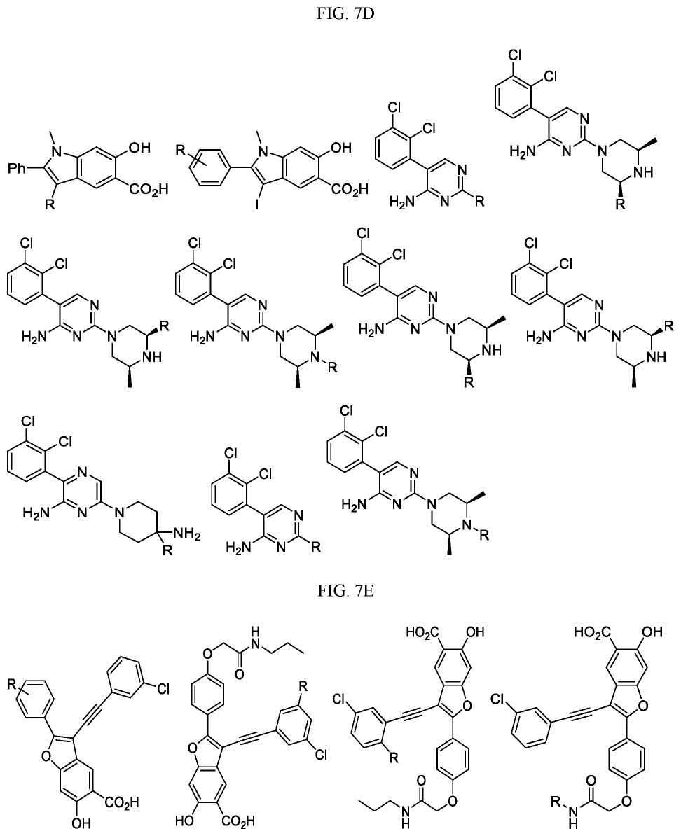

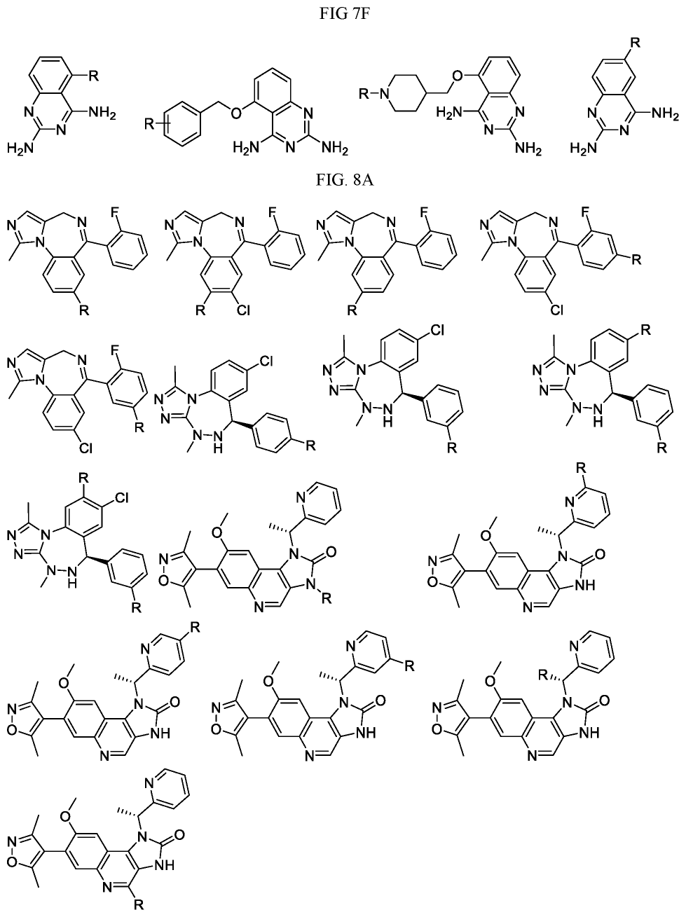

In certain aspects, the Targeting Ligand is a ligand which covalently or non-covalently binds to a Target Protein which has been selected for proteasomal degradation by the selected Degrader. A Targeting Ligand is a molecule or moiety (for example a peptide, nucleotide, antibody, antibody fragment, aptamer, biomolecule or other chemical structure) that binds to a Target Protein, and wherein the Target Protein is a mediator of disease in a host as described in detail below. Exemplary Target Ligands are provided in FIGS. 1A-63.

In certain embodiments, the Targeting Ligand binds to an endogenous protein which has been selected for degradation as a means to achieve a therapeutic effect on the host. Illustrative Targeting Ligands include: RXR ligands, DHFR ligands, Hsp90 inhibitors, kinase inhibitors, HDM2 and MDM2 inhibitors, compounds targeting Human BET bromodomain-containing proteins, HDAC inhibitors, ligands of MerTK, ligands of IDH1, ligands of Mcl-1, ligands of SMRCA2, ligands of EGFR, ligands of RAF, ligands of cRAF, human lysine methyltransferase inhibitors, angiogenesis inhibitors, nuclear hormone receptor compounds, immunosuppressive compounds, and compounds targeting the aryl hydrocarbon receptor (AHR), among numerous others. Targeting Ligands also considered to include their pharmaceutically acceptable salts, prodrugs and isotopic derivatives.

In certain aspects, the Targeting Ligand binds to a dehalogenase enzyme in a patient or subject or in a diagnostic assay and is a haloalkane (preferably a C₁-C₁₀ alkyl group which is substituted with at least one halo group, preferably a halo group at the distal end of the alkyl group (i.e., away from the Linker). In still other embodiments, the Targeting Ligand is a haloalkyl group, wherein said alkyl group generally ranges in size from about 1 or 2 carbons to about 12 carbons in length, often about 2 to 10 carbons in length, often about 3 carbons to about 8 carbons in length, more often about 4 carbons to about 6 carbons in length. The haloalkyl groups are generally linear alkyl groups (although branched-chain alkyl groups may also be used) and are end-capped with at least one halogen group, preferably a single halogen group, often a single chloride group. Haloalkyl PT, groups for use in the present invention are preferably represented by the chemical structure —(CH₂)ᵥ-Halo where v is any integer from 2 to about 12, often about 3 to about 8, more often about 4 to about 6. Halo may be any halogen, but is preferably Cl or Br, more often Cl.

In certain embodiments, the Targeting Ligand is a retinoid X receptor (RXR) agonist or antagonist. Non-limiting examples include retinol, retinoic acid, bexarotene, docosahexenoic acid, compounds disclosed in WO 9929324, the publication by Canan Koch et al. (J. Med. Chem. 1996, 39, 3229-3234) titled “Identification of the First Retinoid X Receptor Homodimer Antagonist”, WO 9712853, EP 0947496A1, WO 2016002968, and analogs thereof.

In certain embodiments, the Targeting Ligand is a DHFR agonist or antagonist. Non-limiting examples include folic acid, methotrexate, 8,10-dideazatetrahydrofolate compounds disclosed by Tian et al. (Chem. Biol. Drug Des. 2016, 87, 444-454) titled “Synthesis, Antifolate and Anticancer Activities of N5-Substituted 8,10-Dideazatetrahydrofolate Analogues”, compounds prepared by Kaur et al. (Biorg. Med. Chem. Lett. 2016, 26, 1936-1940) titled “Rational Modification of the Lead Molecule: Enhancement in the Anticancer and Dihydrofolate Reductase Inhibitory Activity”, WO 2016022890, compounds disclosed by Zhang et al. (Int. J. Antimicrob. Agents 46, 174-182) titled “New Small-Molecule Inhibitors of Dihydrofolate Reductase Inhibit Streptococcus mutans”, modified trimethoprim analogs developed by Singh et al. (J. Med. Chem. 2012, 55, 6381-6390) titled “Mechanism Inspired Development of Rationally Designed Dihydrofolate Reductase Inhibitors as Anticancer Agents”, WO20111153310, and analogs thereof.

In certain embodiments, the Targeting Ligand derived from estrogen, an estrogen analog, SERM (selective estrogen receptor modulator), a SERD (selective estrogen receptor degrader), a complete estrogen receptor degrader, or another form of partial or complete estrogen antagonist or agonist. Examples are the partial anti-estrogens raloxifene and tamoxifen and the complete antiestrogen fulvestrant. Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703,810, as well as US 2015/0005286, WO 2014/205136, and WO 2014/205138. Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone. Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684.

In certain embodiments, the Targeting Ligand is a HSP90 inhibitor identified in Vallee et al. (J. Med. Chem. 2011, 54, 7206-7219) titled “Tricyclic Series of Heat Shock Protein 90 (Hsp90) Inhibitors Part I: Discovery of Tricyclic Imidazo[4,5-C]Pyridines as Potent Inhibitors of the Hsp90 Molecular Chaperone”, including YKB (N-[4-(3H-imidazo[4,5-C]Pyridin-2-yl)-9H-Fluoren-9-yl]-succinamide), a HSP90 inhibitors (modified) identified in Brough et al. (J. Med. Chem. 2008, 51, 196-218) titled “4,5-Diarylisoxazole Hsp90 Chaperone Inhibitors: Potential Therapeutic Agents for the Treatment of Cancer”, including compound 2GJ (5-[2,4-dihydroxy-5-(1-methylethyl)phenyl]-n-ethyl-4-[4-(morpholin-4-ylmethyl)phenyl]isoxazole-3-carboxamide), the HSP90 inhibitor geldanamycin ((4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1] (derivatized) or any of its derivatives (e.g. 17-alkylamino-17-desmethoxygeldanamycin (“17-AAG”) or 17-(2-dimethylaminoethyl)amino-17-desmethoxygeldanamycin (“17-DMAG”)), or a HSP90 inhibitor (modified) identified in Wright et al. (Chem. Biol. 2004, 11, 775-785) titled “Structure-Activity Relationships in Purine-Based Inhibitor Binding to Hsp90 Isoforms”, including the HSP90 inhibitor PU3. Other non-limiting examples of Hsp90 Targeting Ligands include SNX5422 currently in phase I clinical trials Reddy et al. (Clin. Lymphoma Myeloma Leuk. 2013, 13, 385-391) titled “Phase I Trial of the Hsp90 Inhibitor Pf-04929113 (Snx5422) in Adult Patients with Recurrent, Refractory Hematologic Malignancies”, or NVP-AUY922 whose anti-cancer activity was assessed by Jensen et al. (Breast Cancer Research: BCR 2008, 10, R33-R33) titled “Nvp-Auy922: A Small Molecule Hsp90 Inhibitor with Potent Antitumor Activity in Preclinical Breast Cancer Models”.

In certain embodiments, the Targeting Ligand is a kinase inhibitor identified in Millan et al. (J. Med. Chem. 2011, 54, 7797-7814) titled “Design and Synthesis of Inhaled P38 Inhibitors for the Treatment of Chronic Obstructive Pulmonary Disease”, including the kinase inhibitors Y1W and Y1X, a kinase inhibitor identified in Schenkel et al. (J. Med. Chem. 2011, 54, 8440-8450) titled “Discovery of Potent and Highly Selective Thienopyridine Janus Kinase 2 Inhibitors”, including the compounds 6TP and OTP, a kinase inhibitor identified in van Eis et al. (Biorg. Med. Chem. Lett. 2011, 21, 7367-7372) titled “2,6-Naphthyridines as Potent and Selective Inhibitors of the Novel Protein Kinase C Isozymes”, including the kinase inhibitors 07U and YCF identified in Lountos et al. (J. Struct. Biol. 2011, 176, 292-301) titled “Structural Characterization of Inhibitor Complexes with Checkpoint Kinase 2 (Chk2), a Drug Target for Cancer Therapy”, including the kinase inhibitors XK9 and NXP, afatinib, fostamatinib, gefitinib, lenvatinib, vandetanib, Gleevec, pazopanib, AT-9283, TAE684, nilotanib, NVP-BSK805, crizotinib, JNJ FMS, foretinib, OSI-027, OSI-930, or OSI-906.

In certain embodiments, the Targeting Ligand is a HDM2/MDM2 inhibitor identified in Vassilev et al. (Science 2004, 303, 844-848) titled “In Vivo Activation of the P53 Pathway by Small-Molecule Antagonists of Mdm2”, and Schneekloth et al. (Bioorg. Med. Chem. Lett. 2008, 18, 5904-5908) titled “Targeted Intracellular Protein Degradation Induced by a Small Molecule: En Route to Chemical Proteomics”, including the compounds nutlin-3, nutlin-2, and nutlin-1.

In certain embodiments, the Targeting Ligand is a Human BET Bromodomain Targeting Ligand identified in Filippakopoulos et al. (Nature 2010, 468, 1067-1073) titled “Selective Inhibition of Bet Bromodomains” such as JQ1; a ligand identified in Nicodeme et al. (Nature 2010, 468, 1119-1123) titled “Suppression of Inflammation by a Synthetic Histone Mimic”; Chung et al. (J. Med. Chem. 2011, 54, 3827-3838) titled “Discovery and Characterization of Small Molecule Inhibitors of the Bet Family Bromodomains”; a compound disclosed in Hewings et al. (J. Med. Chem. 2011, 54, 6761-6770) titled “3,5-Dimethylisoxazoles Act as Acetyl-Lysine-Mimetic Bromodomain Ligands”; a ligand identified in Dawson et al. (Nature 2011, 478, 529-533) titled “Inhibition of Bet Recruitment to Chromatin as an Effective Treatment for MLL-Fusion Leukaemia”; or a ligand identified in the following patent applications US 2015/0256700, US 2015/0148342, WO 2015/074064, WO 2015/067770, WO 2015/022332, WO 2015/015318, and WO 2015/011084.

In certain embodiments, the Targeting Ligand is a HDAC Targeting Ligand identified in Finnin et al. (Nature 1999, 401, 188-193) titled “Structures of a Histone Deacetylase Homologue Bound to the Tsa and Saha Inhibitors”, or a ligand identified as Formula (I) in PCT WO0222577.

In certain embodiments, the Targeting Ligand is a Human Lysine Methyltransferase ligand identified in Chang et al. (Nat Struct Mol Biol 2009, 16, 312-317) titled “Structural Basis for G9a-Like Protein Lysine Methyltransferase Inhibition by Bix-01294”, a ligand identified in Liu et al. (J. Med. Chem 2009, 52, 7950-7953) titled “Discovery of a 2,4-Diamino-7-Aminoalkoxyquinazoline as a Potent and Selective Inhibitor of Histone Lysine Methyltransferase G9a”, azacitidine, decitabine, or an analog thereof.

In certain embodiments, the Targeting Ligand is an angiogenesis inhibitor. Non-limiting examples of angiogenesis inhibitors include: GA-1, estradiol, testosterone, ovalicin, fumagillin, and analogs thereof.

In certain embodiments, the Targeting Ligand is an immunosuppressive compound. Non-limiting examples of immunosuppressive compounds include: AP21998, hydrocortisone, prednisone, prednisolone, methylprednisolone, beclometasone dipropionate, methotrexate, ciclosporin, tacrolimus, actinomycin, and analogues thereof.

In certain embodiments, the Targeting Ligand is an Aryl Hydrocarbon Receptor (AHR) ligand. Non-limiting examples of AHR ligands include: apigenin, SR1, LGC006, and analogues thereof.

In certain embodiments, the Targeting Ligand is a MerTK or Mer Targeting ligand. Non-limiting examples of MerTK Targeting Ligands are included in WO2013/177168 and WO2014/085225, both titled “Pyrimidine Compounds for the Treatment of Cancer” filed by Wang, et al.

In certain embodiments, the Targeting Ligand is an EGFR ligand. In certain embodiments the Targeting Ligand is an EGRF ligand selected from Afatinib, Dacomitinib, Neratinib, Poziotinib, and Canertinib, or derivatives thereof.

In certain embodiments, the Targeting Ligand is a FLT3 Ligand. In certain embodiments, the Targeting Ligand is a FLT3 ligand selected from Tandutinib, Lestaurtinib, Sorafenib, Midostaurin, Quizartinib, and Crenolanib.

In certain embodiments, the Targeting Ligand is a RAF inhibitor. In certain embodiments the Targeting Ligand is a RAF inhibitor selected from Dabrafenib, Regorafenib, and Vemurafenib. In certain embodiments the Targeting Ligand is a cRAF inhibitor.

In some embodiments, the Targeting Ligand is an Ubc9 SUMO E2 ligase 5F6D Targeting Ligand including but not limited to those described in “Insights Into the Allosteric Inhibition of the SUMO E2 Enzyme Ubc9.” by Hewitt, W. M., et. al. (2016) Angew. Chem. Int. Ed. Engl. 55: 5703-5707

In another embodiment, the Targeting Ligand is a Tank1 Targeting Ligand including but not limited to those described in “Structure of human tankyrase 1 in complex with small-molecule inhibitors PJ34 and XAV939.” Kirby, C. A., Cheung, A., Fazal, A., Shultz, M. D., Stams, T, (2012) Acta Crystallogr., Sect. F 68: 115-118; and “Structure-Efficiency Relationship of [1,2,4]Triazol-3-ylamines as Novel Nicotinamide Isosteres that Inhibit Tankyrases.” Shultz, M. D., et al. (2013) J. Med. Chem. 56: 7049-7059.

In another embodiment, the Targeting Ligand is a SH2 domain of pp60 Src Targeting Ligand including but not limited to those described in “Requirements for Specific Binding of Low Affinity Inhibitor Fragments to the SH2 Domain of pp60Src Are Identical to Those for High Affinity Binding of Full Length Inhibitors,” Gudrun Lange, et al., J. Med. Chem. 2003, 46, 5184-5195.

In another embodiment, the Targeting Ligand is a Sec7 domain Targeting Ligand including but not limited to those described in “The Lysosomal Protein Saposin B Binds Chloroquine,” Huta, B. P., et al., (2016) Chemmedchem 11: 277.

In another embodiment, the Targeting Ligand is a Saposin-B Targeting Ligand including but not limited to those described in “The structure of cytomegalovirus immune modulator UL 141 highlights structural Ig-fold versatility for receptor binding” I. Nemcovicova and D. M. Zajonc Acta Cryst. (2014). D70, 851-862.

In another embodiment, the Targeting Ligand is a Protein 5100-A7 20WS Targeting Ligand including but not limited to those described in “2WOS STRUCTURE OF HUMAN S100A7 IN COMPLEX WITH 2,6 ANS” DOI: 10.2210/pdb2wos/pdb; and “Identification and Characterization of Binding Sites on S100A7, a Participant in Cancer and Inflammation Pathways.” Leon, R., Murray, et al., (2009) Biochemistry 48: 10591-10600.

In another embodiment, the Targeting Ligand is a Phospholipase A2 Targeting Ligand including but not limited to those described in “Structure-based design of the first potent and selective inhibitor of human non-pancreatic secretory phospholipase A2” Schevitz, R. W., et al., Nat. Struct. Biol. 1995, 2, 458-465.

In another embodiment, the Targeting Ligand is a PHIP Targeting Ligand including but not limited to those described in “A Poised Fragment Library Enables Rapid Synthetic Expansion Yielding the First Reported Inhibitors of PHIP(2), an Atypical Bromodomain” Krojer, T.; et al. Chem. Sci. 2016, 7, 2322-2330.

In another embodiment, the Targeting Ligand is a PDZ Targeting Ligand including but not limited to those described in “Discovery of Low-Molecular-Weight Ligands for the AF6 PDZ Domain” Mangesh Joshi, et al. Angew. Chem. Int. Ed. 2006, 45, 3790-3795.

In another embodiment, the Targeting Ligand is a PARP15 Targeting Ligand including but not limited to those described in “Structural Basis for Lack of ADP-ribosyltransferase Activity in Poly(ADP-ribose) Polymerase-13/Zinc Finger Antiviral Protein.” Karlberg, T., et al., (2015) J. Biol. Chem. 290: 7336-7344.

In another embodiment, the Targeting Ligand is a PARP14 Targeting Ligand including but not limited to those described in “Discovery of Ligands for ADP-Ribosyltransferases via Docking-Based Virtual Screening.” Andersson, C. D., et al., (2012) J. Med. Chem. 55: 7706-7718.; “Family-wide chemical profiling and structural analysis of PARP and tankyrase inhibitors.” Wahlberg, E., et al. (2012) Nat. Biotechnol. 30: 283-288.; “Discovery of Ligands for ADP-Ribosyltransferases via Docking-Based Virtual Screening.” Andersson, C. D., et al. (2012) J. Med. Chem. 55: 7706-7718.

In another embodiment, the Targeting Ligand is a MTH1 Targeting Ligand including but not limited to those described in “MTH1 inhibition eradicates cancer by preventing sanitation of the dNTP pool” Helge Gad, et. al. Nature, 2014, 508, 215-221.

In another embodiment, the Targeting Ligand is a mPGES-1 Targeting Ligand including but not limited to those described in “Crystal Structures of mPGES-1 Inhibitor Complexes Form a Basis for the Rational Design of Potent Analgesic and Anti-Inflammatory Therapeutics.” Luz, J. G., et al., (2015) J. Med. Chem. 58: 4727-4737.

In another embodiment, the Targeting Ligand is a FLAP-5-lipoxygenase-activating protein Targeting Ligand including but not limited to those described in “Crystal structure of inhibitor-bound human 5-lipoxygenase-activating protein,” Ferguson, A. D., McKeever, B. M., Xu, S., Wisniewski, D., Miller, D. K., Yamin, T. T., Spencer, R. H., Chu, L., Ujjainwalla, F., Cunningham, B. R., Evans, J. F., Becker, J. W. (2007) Science 317: 510-512.

In another embodiment, the Targeting Ligand is a FA Binding Protein Targeting Ligand including but not limited to those described in “A Real-World Perspective on Molecular Design.” Kuhn, B.; et al. J. Med. Chem. 2016, 59, 4087-4102.

In another embodiment, the Targeting Ligand is a BCL2 Targeting Ligand including but not limited to those described in “ABT-199, a potent and selective BCL-2 inhibitor, achieves antitumor activity while sparing platelets.” Souers, A. J., et al. (2013) NAT. MED. (N.Y.) 19: 202-208.

In another embodiment, the Targeting Ligand is a NF2L2 Targeting Ligand.

In another embodiment, the Targeting Ligand is a CTNNB1 Targeting Ligand.

In another embodiment, the Targeting Ligand is a CBLB Targeting Ligand.

In another embodiment, the Targeting Ligand is a BCL6 Targeting Ligand.

In another embodiment, the Targeting Ligand is a RASK Targeting Ligand.

In another embodiment, the Targeting Ligand is a TNIK Targeting Ligand.

In another embodiment, the Targeting Ligand is a MEN1 Targeting Ligand.

In another embodiment, the Targeting Ligand is a PI3Ka Targeting Ligand.

In another embodiment, the Targeting Ligand is a IDO1 Targeting Ligand.

In another embodiment, the Targeting Ligand is a MCL1 Targeting Ligand.

In another embodiment, the Targeting Ligand is a PTPN2 Targeting Ligand.

In another embodiment, the Targeting Ligand is a HER2 Targeting Ligand.

In another embodiment, the Targeting Ligand is an EGFR Targeting Ligand. In certain embodiments the Targeting Ligand is selected from erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer). The linker can be placed on these Targeting Ligands in any location that does not interfere with the Ligands binding to EGFR. Non-limiting examples of Linker binding locations are provided in the below tables. In certain embodiments, the EGFR Targeting Ligand binds the L858R mutant of EGFR. In another embodiment, the EGFR Targeting Ligand binds the T790M mutant of EGFR. In another embodiment, the EGFR Targeting Ligand binds the C797G or C797S mutant of EGFR. In certain embodiments, the EGFR Targeting Ligand is selected from erlotinib, gefitinib, afatinib, neratinib, and dacomitinib and binds the L858R mutant of EGFR. In another embodiment, the EGFR Targeting Ligand is selected from osimertinib, rociletinib, olmutinib, naquotinib, nazartinib, PF-06747775, Icotinib, Neratinib, Avitinib, Tarloxotinib, PF-0645998, Tesevatinib, Transtinib, WZ-3146, WZ8040, and CNX-2006 and binds the T790M mutant of EGFR. In another embodiment, the EGFR Targeting Ligand is EAI045 and binds the C797G or C797S mutant of EGFR.

In certain embodiments, the protein target and Targeting Ligand pair are chosen by screening a library of ligands. Such a screening is exemplified in “Kinase Inhibitor Profiling Reveals Unexpected Opportunities to Inhibit Disease-Associated Mutant Kinases” by Duong-Ly et al.; Cell Reports 14, 772-781 Feb. 2, 2016.

In certain embodiments, the protein target and Targeting Ligand pair are discovered by screening promiscuous kinase binding ligands for context-specific degradation. Non-limiting examples of targeting ligands are shown below and are found in “Optimized Chemical Proteomics Assay for Kinase Inhibitor Profiling” Guillaume Medard, Fiona Pachl, Benjamin Ruprecht, Susan Klaeger, Stephanie Heinzlmeir, Dominic Helm, Huichao Qiao, Xin Ku, Mathias Wilhelm, Thomas Kuehne, Zhixiang Wu, Antje Dittmann, Carsten Hopf, Karl Kramer, and Bernhard Kuster J. Proteome Res., 2015, 14(3), pp 1574-1586:

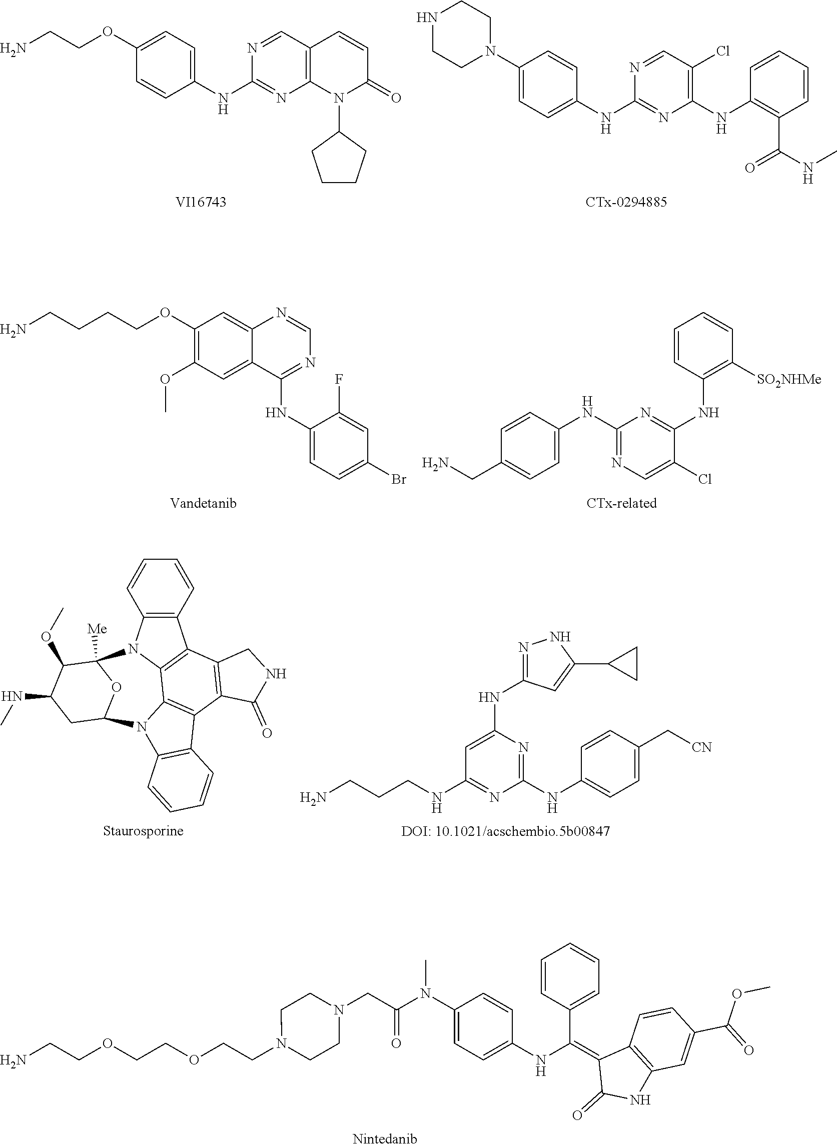

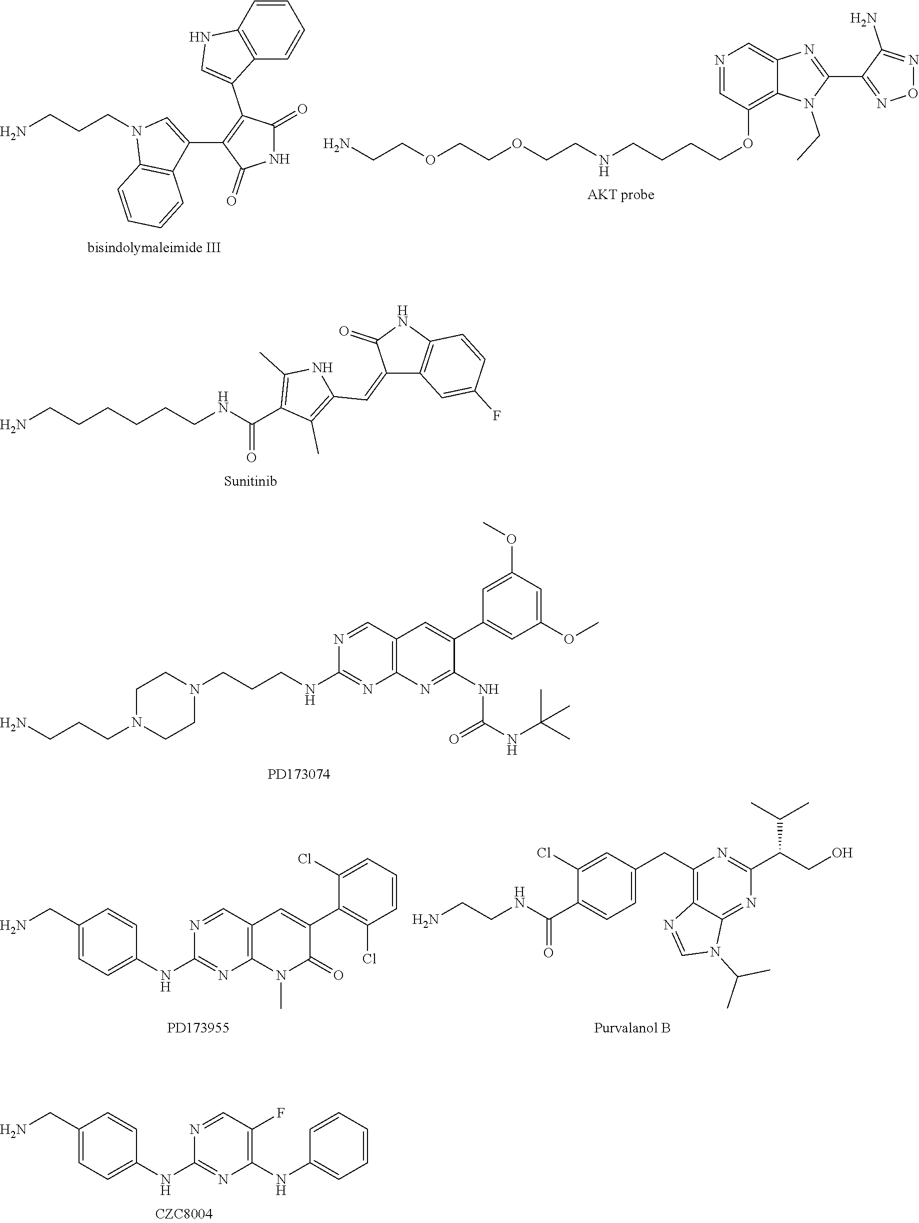

These ligands can be attached to linkers as shown below:

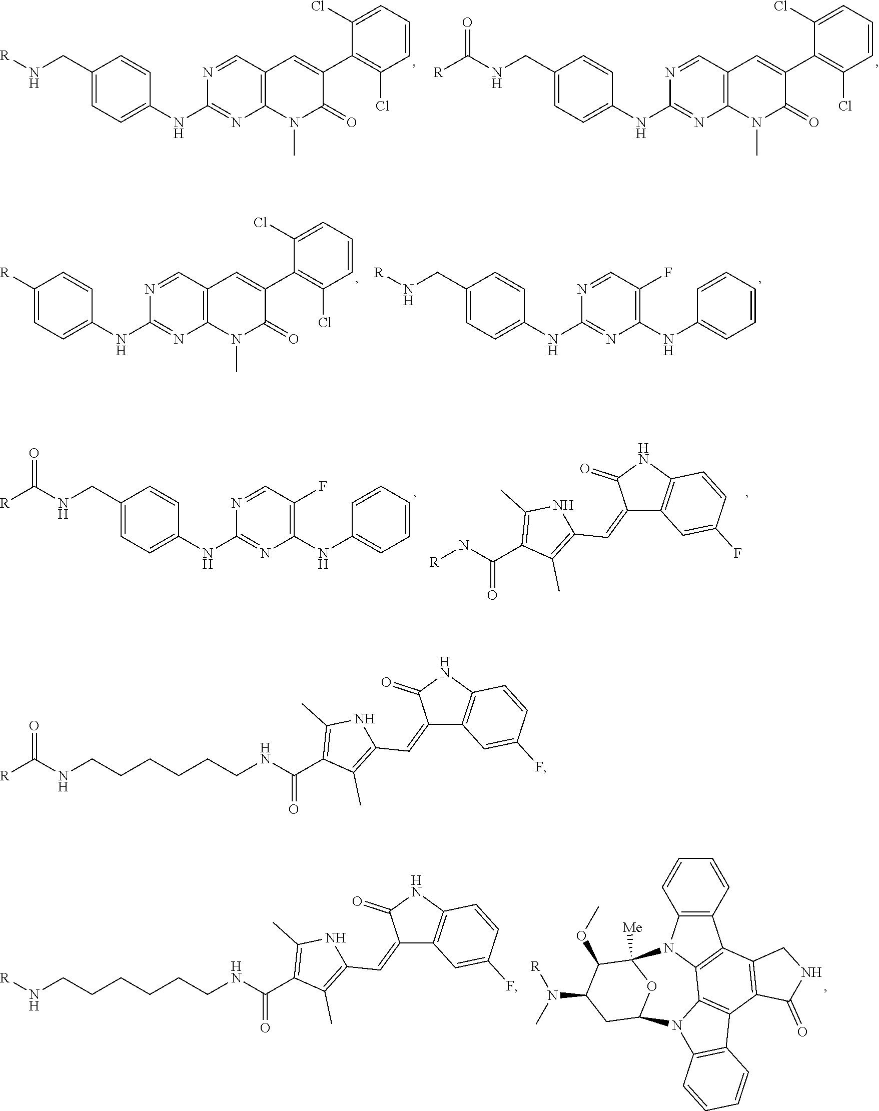

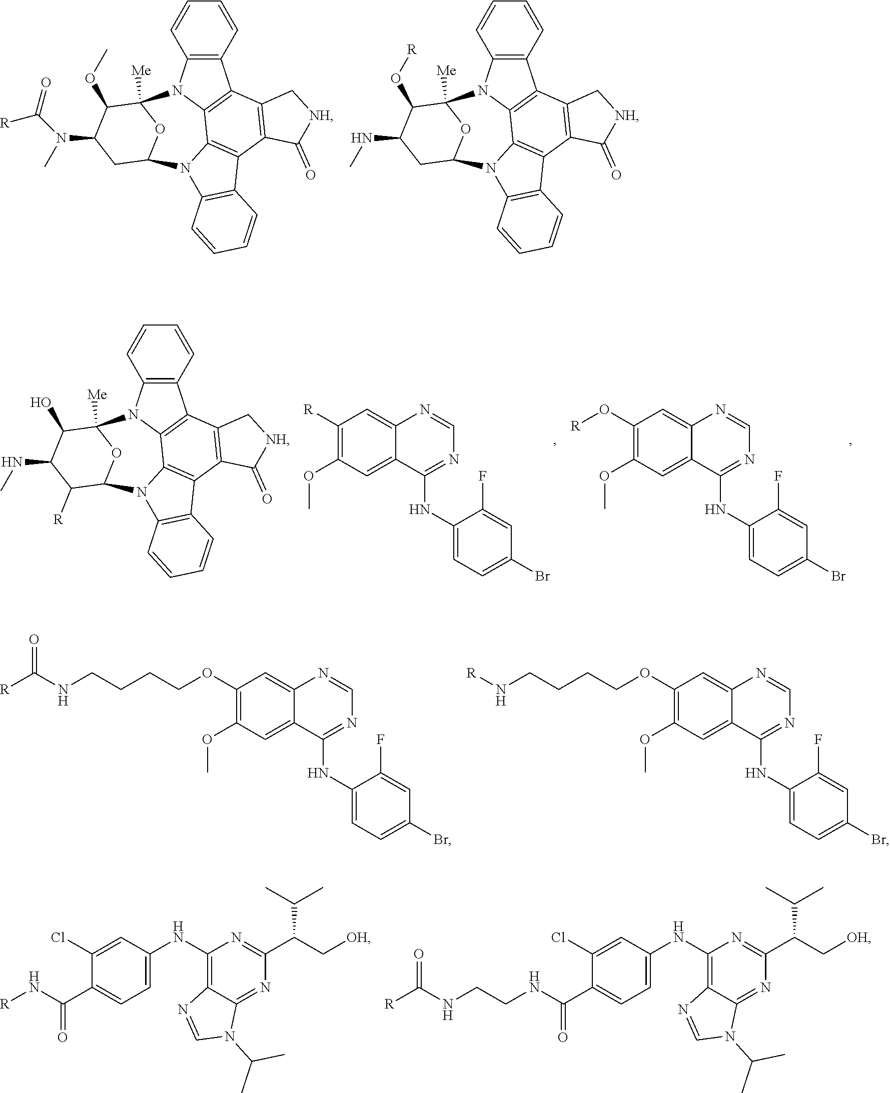

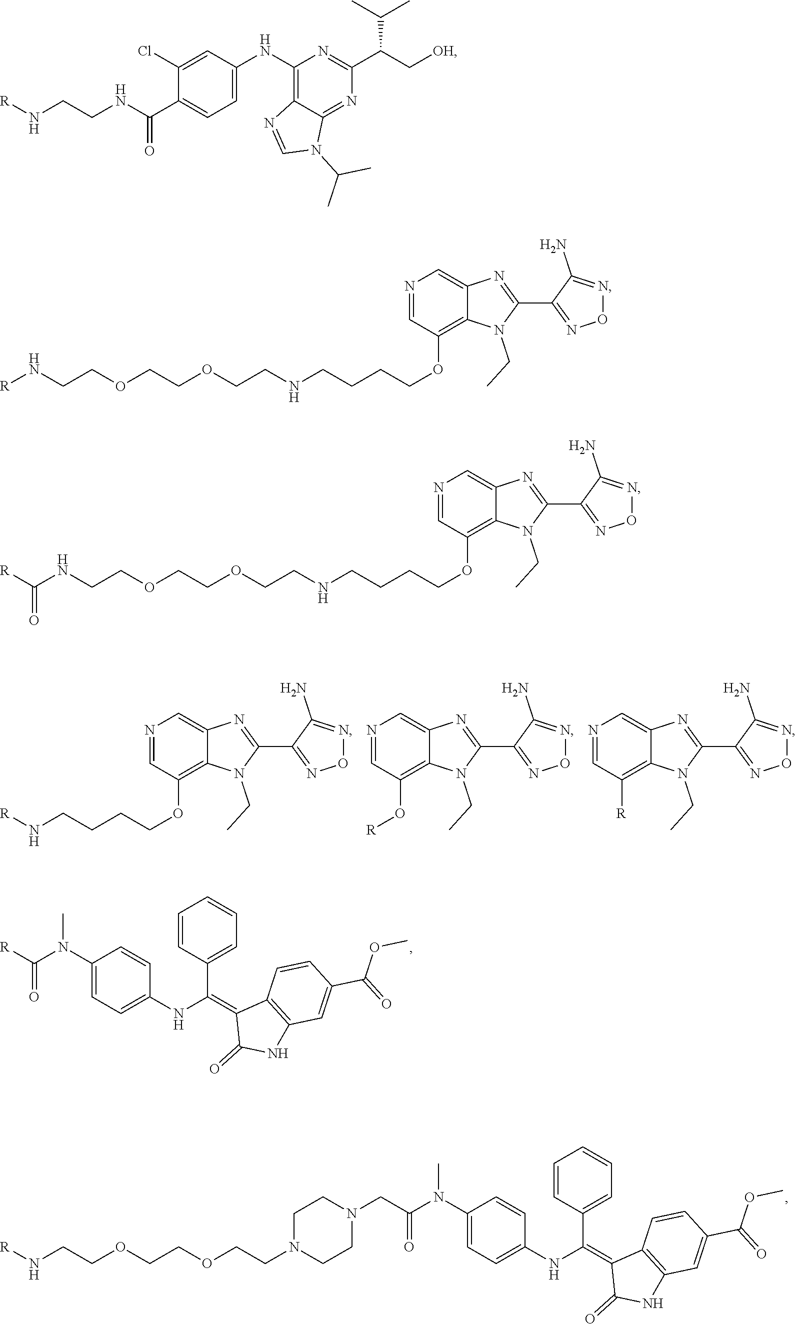

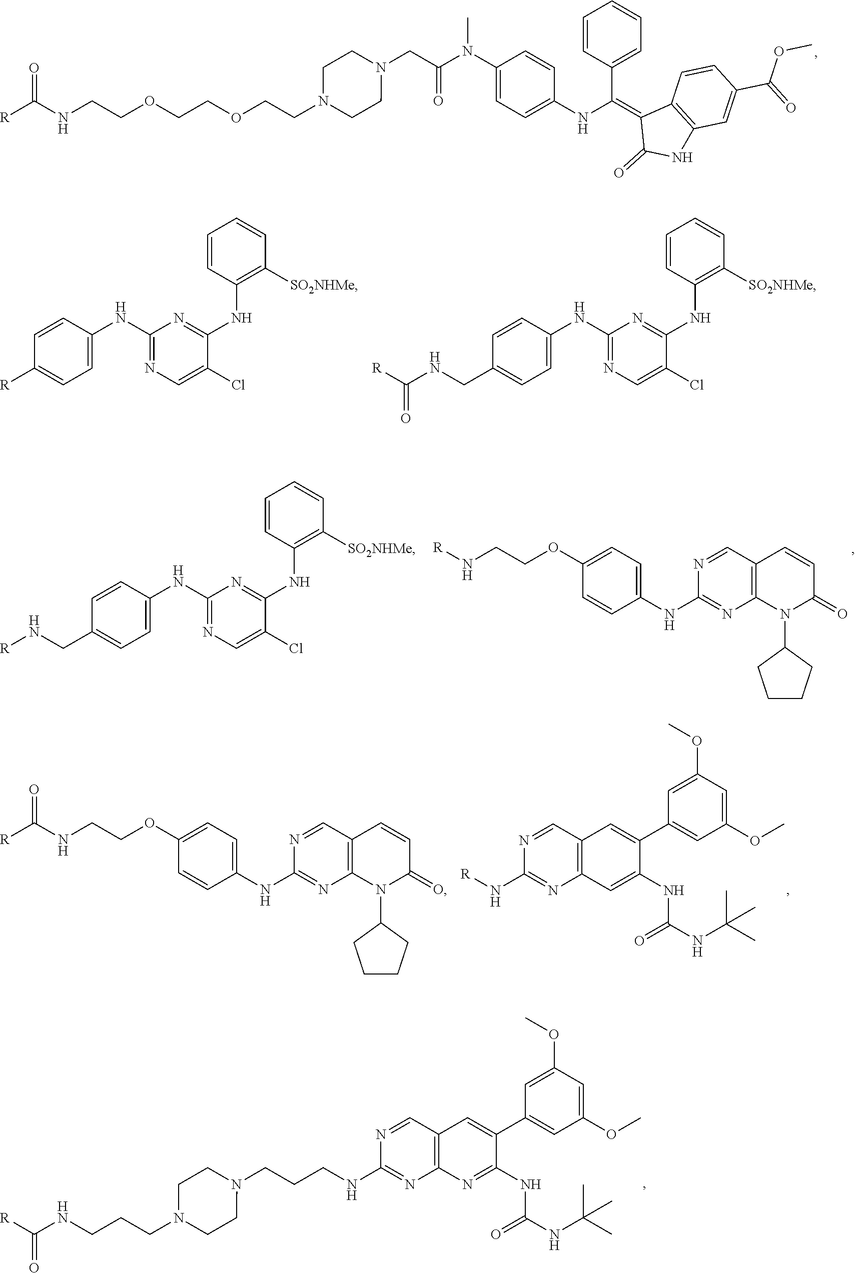

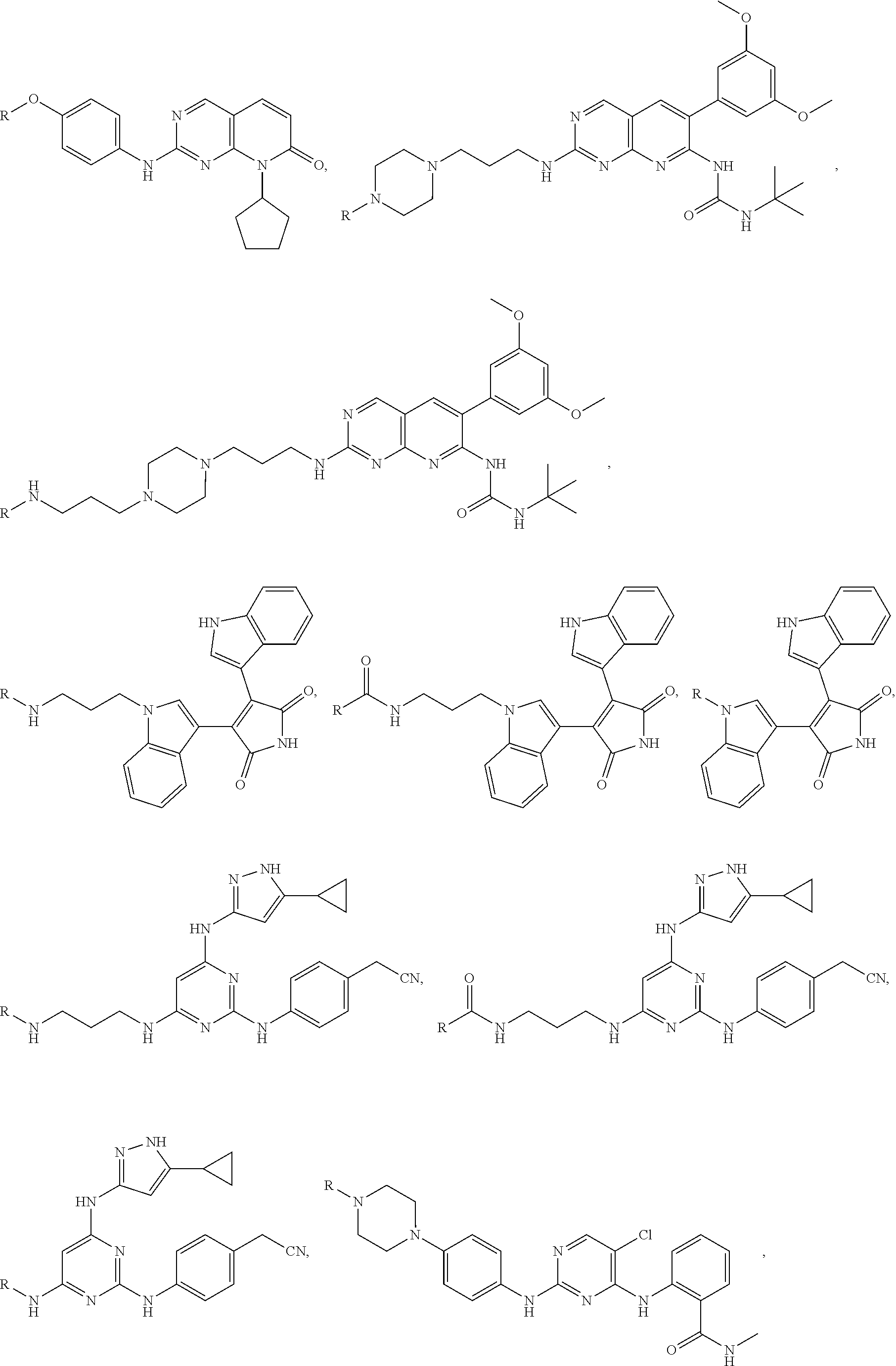

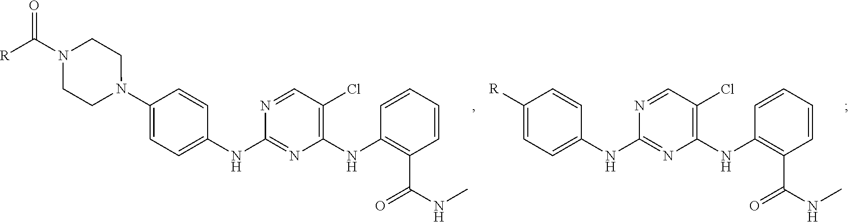

wherein:
R represents exemplary points at which the Spacer is attached.

In an alternative embodiment, the Targeting Ligand is selected from a DOTL1-Ligand, a CBP Ligand, an ERK1 Ligand, an ERK2 Ligand, a JAK2 Ligand, an FGFR3 Ligand, an FGFR4 Ligand, a WDR5 Ligand, a PAK4 Ligand, a BRAF Ligand, a KRAS Ligand, a BTK Ligand, and a SHOC2 Ligand. In another alternative embodiment, the Targeting Ligand is selected from a UCHL1 Ligand, a USP1 Ligand, a USP2 Ligand, a USP4 Ligand, a USP6 Ligand, a USP7 Ligand, a USP8 Ligand, a USP9x Ligand, a USP10 Ligand, a USP 11 Ligand, a USP13 Ligand, a USP14 Ligand, a USP17 Ligand, and a USP28 Ligand.

According to the present invention, the Targeting Ligand can be covalently bound to the Linker in any manner that achieves the desired results of the Degrader for therapeutic use. In certain non-limiting embodiments, the Targeting Ligand is bound to the Linker with a functional group that does not adversely affect the binding of the Ligand to the Target Protein. The attachment points below are exemplary in nature and one of ordinary skill in the art would be able to determine different appropriate attachment points.

The non-limiting compounds described below exemplify some of the members of these types of Targeting Ligands.

In certain embodiments, the Targeting Ligand binds to ASH1L. For example, the ASH1L small molecule inhibitor may be as described in WO2017/197240, the entirety of which is incorporated herein by reference. In certain embodiments, the Targeting Ligand is

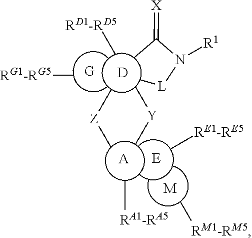

wherein all variables are as defined in WO2017/197240. As described in the '240 application, in some embodiments, any of formulas provided therein may be converted to bifunctional compounds composed of ASH1L inhibitor and an E3 ubiquitin ligase ligand connected with a linker, which function to bind ASH1L and recruit an E ubiquitin ligase (Cereblon, VHL ligase, etc.) complex to ubiquitinate and induce proteasome-mediated degradation of ASH1L. In the present invention, the linker is a Linker as defined herein covalently bound to a Degron as described herein.

In an alternative embodiment, the Targeting Ligand is a deubiquitylating enzyme (DUB) inhibitor as described in WO2018/065768, WO2018/060742, WO2018/060691, WO2018/060689, WO2017/163078, WO2017/158388, WO2017/158381, WO2017/141036, WO2018/103614, WO2017/093718, WO2017/009650, WO2016/156816, or WO2016/046530.

In an alternative embodiment, any of the Targeting Ligands as described herein may be optionally substituted with one or more, for example 1, 2, 3, 4, or 5, groups selected from R¹⁰¹.

### VI. Methods of Treatment

The compounds of Formula I, Formula II, or Formula III can be used in an effective amount to treat a host, including a human, in need thereof, optionally in a pharmaceutically acceptable carrier to treat any of the disorders described herein.

The terms “treat”, “treating”, and “treatment”, etc., as used herein, refer to any action providing a benefit to a patient for which the present compounds may be administered, including the treatment of any disease state or condition which is modulated through the protein to which the present compounds bind. Illustrative non-limiting disease states or conditions, including cancer, which may be treated using compounds according to the present invention are set forth hereinabove.

The compounds of Formula I, Formula II, or Formula III and compositions as described herein can be used to degrade a Target Protein which is a mediator of the disorder affecting the patient, such as a human. The control of protein level afforded by the Formula I, Formula II, or Formula III compounds of the present invention provides treatment of a disease state or condition, which is modulated through the Target Protein by lowering the level of that protein in the cell, e.g., cell of a patient. In certain embodiments, the method comprises administering an effective amount of the compound as described herein, optionally including a pharmaceutically acceptable excipient, carrier, adjuvant, i.e., a pharmaceutically acceptable composition, optionally in combination with another bioactive agent or combination of agents.

The term “disease state or condition” when used in connection with a Formula I, Formula II, or Formula III compound is meant to refer to any disease state or condition wherein protein dysregulation (i.e., the amount of protein expressed in a patient is elevated) occurs via a Target Protein and where degradation of such protein in a patient may provide beneficial therapy or relief of symptoms to a patient in need thereof. In certain instances, the disease state or condition may be cured. The compounds of Formula I, Formula II, or Formula III are for example useful as therapeutic agents when administered in an effective amount to a host, including a human, to treat a myelo- or lymphoproliferative disorder such as B- or T-cell lymphomas, multiple myeloma, Waldenstrom's macroglobulinemia, Wiskott-Aldrich syndrome, or a post-transplant lymphoproliferative disorder; an immune disorder, including autoimmune disorders such as Addison disease, Celiac disease, dermatomyositis, Graves disease, thyroiditis, multiple sclerosis, pernicious anemia, reactive arthritis, lupus, or type I diabetes; a disease of cardiologic malfunction, including hypercholesterolemia; an infectious disease, including viral and/or bacterial infections; an inflammatory condition, including asthma, chronic peptic ulcers, tuberculosis, rheumatoid arthritis, periodontitis, ulcerative colitis, Crohn's disease, or hepatitis.

The term “disease state or condition” when used in connection with a Formula V, Formula VI, Formula VII, Formula VIII, or Formula XII compound for example, refers to any therapeutic indication which can be treated by decreasing the activity of cereblon or a cereblon-containing E3 Ligase, including but not limited to uses known for the cereblon binders thalidomide, pomalidomide or lenalidomide. Nonlimiting examples of uses for cereblon binders are multiple myeloma, a hematological disorder such as myelodysplastic syndrome, cancer, tumor, abnormal cellular proliferation, HIV/AIDS, HBV, HCV, hepatitis, Crohn's disease, sarcoidosis, graft-versus-host disease, rheumatoid arthritis, Behcet's disease, tuberculosis, and myelofibrosis. Other indications include a myelo- or lymphoproliferative disorder such as B- or T-cell lymphomas, Waldenstrom's macroglobulinemia, Wiskott-Aldrich syndrome, or a post-transplant lymphoproliferative disorder; an immune disorder, including autoimmune disorders such as Addison disease, Celiac disease, dermatomyositis, Graves disease, thyroiditis, multiple sclerosis, pernicious anemia, arthritis, and in particular rheumatoid arthritis, lupus, or type I diabetes; a disease of cardiologic malfunction, including hypercholesterolemia; an infectious disease, including viral and/or bacterial infection, as described generally herein; an inflammatory condition, including asthma, chronic peptic ulcers, tuberculosis, rheumatoid arthritis, periodontitis and ulcerative colitis.

In certain embodiments, the present invention provides for administering a compound of Formula I, Formula II, or Formula III to a patient, for example, a human, having an infectious disease, wherein the therapy targets a protein of the infectious agent, optionally in combination with another bioactive agent. The disease state or condition may be a disease caused by a microbial agent or other exogenous agent such as a virus (as non-limiting examples, HIV, HBV, HCV, HSV, HPV, RSV, CMV, Ebola, Flavivirus, Pestivirus, Rotavirus, Influenza, Coronavirus, EBV, viral pneumonia, drug-resistant viruses, Bird flu, RNA virus, DNA virus, adenovirus, poxvirus, Picornavirus, Togavirus, Orthomyxovirus, Retrovirus or Hepadnovirus), bacteria (Gram-negative, Gram-positive, fungus, protozoa, helminth, worms, prion, parasite, or other microbe or may be a disease state, which is caused by overexpression of a protein, which leads to a disease state and/or condition.

In certain embodiments, the condition treated with a compound of the present invention is a disorder related to abnormal cellular proliferation. Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis. Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic micro-angiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

Cutaneous contact hypersensitivity and asthma are just two examples of immune responses that can be associated with significant morbidity. Others include atopic dermatitis, eczema, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

In one non-limiting embodiment compounds of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

Disease states of conditions which may be treated using compounds according to the present invention include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease 1 (PKD1) or 2 (PKD2) Prader-Willi syndrome, Sickle-cell disease, Tay-Sachs disease, Turner syndrome.

Further disease states or conditions which may be treated by compounds according to the present invention include Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barre syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, Vasculitis.

Still additional disease states or conditions which can be treated by compounds according to the present invention include aceruloplasminemia, Achondrogenesis type II, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis Alstrom syndrome, Alexander disease, Amelogenesis imperfecta, ALA dehydratase deficiency, Anderson-Fabry disease, androgen insensitivity syndrome, Anemia Angiokeratoma Corporis Diffusum, Angiomatosis retinae (von Hippel-Lindau disease) Apert syndrome, Arachnodactyly (Marfan syndrome), Stickler syndrome, Arthrochalasis multiplex congenital (Ehlers-Danlos syndrome #arthrochalasia type) ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dube syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria Erythroid 5-aminolevulinate synthetase deficiency, Erythropoietic porphyria, erythropoietic protoporphyria, erythropoietic uroporphyria, Friedreich's ataxia-familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alstrom syndrome), Vrolik disease, Waardenburg syndrome, Warburg Sjo Fledelius Syndrome, Weissenbacher-Zweymuller syndrome, Wolf-Hirschhorn syndrome, Wolff Periodic disease, Weissenbacher-Zweymuller syndrome and Xeroderma pigmentosum, among others.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitt's Lymphoma, B-cell ALL, Philadelphia chromosome positive ALL and Philadelphia chromosome positive CML.

Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitt's lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

In certain embodiments the cancer is NUT midline carcinoma.

In certain embodiments the cancer is adenoid cystic carcinoma.

The term “bioactive agent” is used to describe an agent, other than a compound according to the present invention, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

### VIII. Combination Therapy

The disclosed compounds described herein can be used in an effective amount alone or in combination with another compound of the present invention or another bioactive agent or second therapeutic agent to treat a patient such as a human with a disorder, including but not limited to those described herein.

The term “bioactive agent” is used to describe an agent, other than the selected compound according to the present invention, which can be used in combination or alternation with a compound of the present invention to achieve a desired result of therapy. In certain embodiments, the compound of the present invention and the bioactive agent are administered in a manner that they are active in vivo during overlapping time periods, for example, have time-period overlapping Cmax, Tmax, AUC or other pharmacokinetic parameter. In another embodiment, the compound of the present invention and the bioactive agent are administered to a patient in need thereof that do not have overlapping pharmacokinetic parameter, however, one has a therapeutic impact on the therapeutic efficacy of the other.

In one aspect of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (Opdivo), pembrolizumab (Keytruda), pidilizumab, AMP-224 (AstraZeneca and MedImmune), PF-06801591 (Pfizer), MEDIO680 (AstraZeneca), PDR001 (Novartis), REGN2810 (Regeneron), SHR-12-1 (Jiangsu Hengrui Medicine Company and Incyte Corporation), TSR-042 (Tesaro), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (Tecentriq), durvalumab (AstraZeneca and MedImmune), KN035 (Alphamab), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). An example of a TIM-3 inhibitor is TSR-022 (Tesaro).

In certain embodiments the checkpoint inhibitor is selected from nivolumab/OPDIVO®; pembrolizumab/KEYTRUDA®; and pidilizumab/CT-011, MPDL3280A/RG7446; MEDI4736; MSB0010718C; BMS 936559, a PDL2/lg fusion protein such as AMP 224 or an inhibitor of B7-H3 (e.g., MGA271), B7-H4, BTLA, HVEM, TIM3, GAL9, LAG 3, VISTA, KIR, 2B4, CD160, CGEN-15049, CHK 1, CHK2, A2aR, B-7 family ligands, or a combination thereof.

In yet another embodiment, one of the active compounds described herein can be administered in an effective amount for the treatment of abnormal tissue of the female reproductive system such as breast, ovarian, endometrial, or uterine cancer, in combination or alternation with an effective amount of an estrogen inhibitor including, but not limited to, a SERM (selective estrogen receptor modulator), a SERD (selective estrogen receptor degrader), a complete estrogen receptor degrader, or another form of partial or complete estrogen antagonist or agonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors.

Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703, 810, as well as US 2015/0005286, WO 2014/205136, and WO 2014/205138.

Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestrant; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone.

Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684.

In another embodiment, an active compounds described herein can be administered in an effective amount for the treatment of abnormal tissue of the male reproductive system such as prostate or testicular cancer, in combination or alternation with an effective amount of an androgen (such as testosterone) inhibitor including, but not limited to a selective androgen receptor modulator, a selective androgen receptor degrader, a complete androgen receptor degrader, or another form of partial or complete androgen antagonist. In certain embodiments, the prostate or testicular cancer is androgen-resistant.

Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine.

In certain embodiments, the bioactive agent is an ALK inhibitor. Examples of ALK inhibitors include but are not limited to Crizotinib, Alectinib, ceritinib, TAE684 (NVP-TAE684), GSK1838705A, AZD3463, ASP3026, PF-06463922, entrectinib (RXDX-101), and AP26113.

In certain embodiments, the bioactive agent is an EGFR inhibitor. Examples of EGFR inhibitors include erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775 (Pfizer), icotinib (BPI-2009), neratinib (HKI-272; PB272); avitinib (AC0010), EAI045, tarloxotinib (TH-4000; PR-610), PF-06459988 (Pfizer), tesevatinib (XL647; EXEL-7647; KD-019), transtinib, WZ-3146, WZ8040, CNX-2006, and dacomitinib (PF-00299804; Pfizer).

In certain embodiments, the bioactive agent is an HER-2 inhibitor. Examples of HER-2 inhibitors include trastuzumab, lapatinib, ado-trastuzumab emtansine, and pertuzumab.

In certain embodiments, the bioactive agent is a CD20 inhibitor. Examples of CD20 inhibitors include obinutuzumab, rituximab, fatumumab, ibritumomab, tositumomab, and ocrelizumab.

In certain embodiments, the bioactive agent is a JAK3 inhibitor. Examples of JAK3 inhibitors include tasocitinib.

In certain embodiments, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl] amino]-3-nitrophenyl]sulfonylbenzamide) (navitoclax), ABT-263 ((R)-4-(4-((4′-chloro-4,4-dimethyl-3,4,5,6-tetrahydro-[1, 1′-biphenyl]-2-yl)methyl)piperazin-1-yl)-N-((4-((4-morpholino-1-(phenylthio)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethylethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (Oblimersen).

In certain embodiments, the bioactive agent is a kinase inhibitor. In certain embodiments, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include, but are not limited to, Wortmannin, demethoxyviridin, perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((+)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3, 5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422).

Examples of BTK inhibitors include ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, but are not limited to, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R¹¹² (3,3′-((5-fluoropyrimidine-2,4-diyl)bis(azanediyl))diphenol), R³⁴⁸ (3-Ethyl-4-methylpyridine), R⁴⁰⁶ (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al. Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In certain embodiments, the bioactive agent is a MEK inhibitor. MEK inhibitors are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-l(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2y1)methyl)benzamide), or AZD8330 (2-((2-fluoro-4-iodophenyl)amino)-N-(2 hydroxyethoxy)-1, 5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088.

In certain embodiments, the bioactive agent is a Raf inhibitor. Raf inhibitors are known and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (Encorafenib).

In certain embodiments, the bioactive agent is an AKT inhibitor, including, but not limited to, MK-2206, GSK690693, Perifosine, (KRX-0401), GDC-0068, Triciribine, AZD5363, Honokiol, PF-04691502, and Miltefosine, a FLT-3 inhibitor, including, but not limited to, P406, Dovitinib, Quizartinib (AC220), Amuvatinib (MP-470), Tandutinib (MLN518), ENMD-2076, and KW-2449, or a combination thereof.

In certain embodiments, the bioactive agent is an mTOR inhibitor. Examples of mTOR inhibitors include, but are not limited to, rapamycin and its analogs, everolimus (Afinitor), temsirolimus, ridaforolimus, sirolimus, and deforolimus. Examples of MEK inhibitors include but are not limited to tametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-l(2H-yl}phenyl)acetamide), selumetinob (6-(4-bromo-2-chloroanilino)-7-fluoro-N-(2-hydroxyethoxy)-3-methylbenzimidazole-5-carboxamide), pimasertib/AS703026/MSC1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol) (cobimetinib), refametinib/BAY869766/RDEA119 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6 carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2 yl)methyl)benzamide), or AZD8330 (2-((2-fluoro-4-iodophenyl)amino)-N-(2-hydroxyethoxy)-1,5-dimethyl-6-oxo-1,6-dihydropyridine-3-carboxamide).

In certain embodiments, the bioactive agent is a RAS inhibitor. Examples of RAS inhibitors include but are not limited to Reolysin and siG12D LODER.

In certain embodiments, the bioactive agent is a HSP inhibitor. HSP inhibitors include but are not limited to Geldanamycin or 17-N-Allylamino-17-demethoxygeldanamycin (17AAG), and Radicicol.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, aFLT-3 inhibitor, a VEGFR inhibitor, an aurora kinase inhibitor, a PIK-1 modulator, an HDAC inhibitor, a c-MET inhibitor, a PARP inhibitor, a Cdk inhibitor, an IGFR-TK inhibitor, an anti-HGF antibody, a focal adhesion kinase inhibitor, a Map kinase kinase (mek) inhibitor, a VEGF trap antibody, pemetrexed, panitumumab, amrubicin, oregovomab, Lep-etu, nolatrexed, azd2171, batabulin, of atumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdRiKRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In certain embodiments the compound is administered in combination with ifosfamide.

In certain embodiments, the bioactive agent is selected from, but are not limited to, Imatinib mesylate (Gleevac®), Dasatinib (Sprycel®), Nilotinib (Tasigna®), Bosutinib (Bosulif®), Trastuzumab (Herceptin®), trastuzumab-DM1, Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In certain aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin®) or liposomal vincristine (Marqibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™).

Examples of additional suitable chemotherapeutic agents include, but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

In some embodiments, the compound of the present invention is administered in combination with a chemotherapeutic agent (e.g., a cytotoxic agent or other chemical compound useful in the treatment of cancer). Examples of chemotherapeutic agents include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Inti. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-1 1); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the compound of the present invention. Suitable dosing regimens of combination chemotherapies are known in the ar. For example combination dosing regimes are described in Saltz et al., Proc. Am. Soc. Clin. Oncol. 18:233a (1999) and Douillard et al., Lancet 355(9209): 1041-1047 (2000).

Additional therapeutic agents that can be administered in combination with a Compound disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In certain embodiments, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR), and trastuzumab targets the human epidermal growth factor receptor 2 (HER-2). MAbs that bind to cell surface growth factor receptors prevent the targeted receptors from sending their normal growth-promoting signals. They may also trigger apoptosis and activate the immune system to destroy tumor cells.

In one aspect of the present invention, the bioactive agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g.ridaforolimus, azathioprine, campath 1H, a SiP receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9. A-3A, 33B3.1, 15-deoxyspergualin, tresperimus, Leflunomide ARAVA®, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA4lg (Abatacept), belatacept, LFA3lg, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In some embodiments, the bioactive agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-l-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); SIMPONI® (golimumab); ILARIS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

In certain embodiments a compound described herein for use in combination with a compound of the present invention is used instead as the Targeting Ligand wherein the attachment point to linker is a suitable location that maintains activity.

The combination therapy may include a therapeutic agent which is a non-drug treatment. For example, the compound could be administered in addition to radiation therapy, cryotherapy, hyperthermia, and/or surgical excision of tumor tissue.

In certain embodiments the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

In certain embodiments the second therapeutic agent is administered on a different dosage schedule than the compound of the present invention. For example the second therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In another embodiment the first therapeutic agent has a treatment holiday. For example the first therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In certain embodiments both the first and second therapeutic have a treatment holiday.

### IX. Pharmaceutical Compositions

The selected compound of Formula I, Formula II, or Formula III as described herein or its pharmaceutically acceptable salt can be administered as the neat chemical, but is more typically administered as a pharmaceutical composition, that includes an effective amount for a patient, typically a human, in need of such treatment for any of the disorders described herein in a pharmaceutically acceptable carrier. The pharmaceutical composition may contain a compound or salt thereof as the only active agent, or, in an alternative embodiment, the compound or its salt and at least one additional active agent for the disease to be treated.

The pharmaceutical compositions of the invention may be administered in a therapeutically effective amount by any desired mode of administration. In certain embodiments, the compound or its pharmaceutically acceptable salt is delivered in an effective amount with a pharmaceutically acceptable carrier for oral delivery. As more general non-limiting examples, the pharmaceutical composition one suitable for oral (including buccal and sub-lingual), rectal, nasal, topical, transdermal, pulmonary, vaginal or parenteral (including intramuscular, intra-arterial, intrathecal, subcutaneous and intravenous), injections, inhalation or spray, intra-aortal, intracranial, subdermal, intraperitoneal, subcutaneous, or by other means of administration containing conventional pharmaceutically acceptable carriers. A typical manner of administration is oral, topical or intravenous, using a convenient daily dosage regimen which can be adjusted according to the degree of affliction.

As these compounds are catalytic, typically less is needed for efficacy than for a corresponding Target Protein inhibitor.

Suitable dosage ranges depend upon numerous factors such as the severity of the disease to be treated, the age and relative health of the subject, the potency of the compound used, the route and form of administration, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

In certain embodiments the pharmaceutical composition is in a dosage form that contains from about 0.001 mg to about 2000 mg, from about 1 mg to about 1000 mg, from about 10 mg to about 800 mg, or from about 20 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least about 0.001, 0.005, 0.01, 0.025, 0.05, 0.1, 1, 5, 10, 25, 50, 100, 200, 250, 300, 400, 500, 600, 700, or 750 mg of active compound, or its salt and at most about 1 gram of active compound or its salt.

In certain embodiments the pharmaceutical composition is in a dosage form that contains from about 0.01 mg to about 1000 mg, from about 0.1 mg to about 750 mg, from about 1 mg to about 500 mg, or from about 5, 10, 15, or 20 mg to about 250 mg of the active compound or its pharmaceutically acceptable salt. Examples are dosage forms are those delivering at least 0.01, 0.05, 0.1, 1, 5, 10, 25, 50, 100, 200, 250, 300, 400, 500, 600, 700, or 750 mg of active compound, or its salt. When the weight is used herein, it can refer to either the compound alone or the compound in combination with its pharmaceutically acceptable salt.

In some embodiments, compounds disclosed herein are administered once a day (QD), twice a day (BID), or three times a day (TID). In some embodiments, compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or indefinitely.

In certain embodiments the compound of the present invention is administered once a day, twice a day, three times a day, or four times a day.

In certain embodiments the compound of the present invention is administered orally once a day. In certain embodiments the compound of the present invention is administered orally twice a day. In certain embodiments the compound of the present invention is administered orally three times a day. In certain embodiments the compound of the present invention is administered orally four times a day.

In certain embodiments the compound or its salt of the present invention is administered intravenously, using a schedule as directed by the healthcare provider. In certain embodiments, the compound is administered at least once a day, once a week, once every two weeks, three weeks, one month or less frequently. In certain embodiments the compound of the present invention is administered intravenously twice a day.

In some embodiments the compound of the present invention is administered with a treatment holiday between treatment cycles. For example the compound may have a treatment holiday of 1 day, 2 days, 3 days, 4 days, 5 days, 6 days, 7 days, 8 days, 9 days, 10 days, 11 days, 12 days, 13 days, 14 days, or even at least three or four weeks off per treatment cycle.

In some embodiments a loading dose is administered to begin treatment. For example, the compound may be administered in a dosage that is at least about 1.5×, 2×, 2.5×, 3×, 3.5×, 4×, 4.5×, 5×, 5.5×, 6×, 6.5×, 7×, 7.5×, 8×, 8.5×, 9×, 9.5×, or 10× higher dose to initiate treatment than the maintenance dose treatment cycle. Additional exemplary loading doses include at least about 1.5×, 2×, 2.5×, 3×, 3.5×, 4×, 4.5×, 5×, 5.5×, 5×, 6.5×, 7×, 7.5×, 8×, 8.5×, 9×, 9.5×, or 10× higher dose on the first 1, 2, 3, 4, 5, 6, 7, 8, 9, or 10 days of treatment followed by the maintenance dose on the remaining days of treatment in the treatment cycle.

The pharmaceutical composition may also include an effective amount of the active compound described herein and an additional active agent, wherein the additional active agent is administered according to its own treatment regimen, or as determined by the healthcare provider, or alternatively synchronized with the compound of the present invention.

In certain embodiments a therapeutic amount may for example be in the range of about 0.0001 mg/kg to about 25 mg/kg body weight. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

In certain embodiments the dose ranges from about 0.001-10 mg/kg of patient bodyweight, for example about 0.0001 mg/kg, about 0.0005 mg/kg, about 0.001 mg/kg, about 0.005 mg/kg, about 0.01 mg/kg, about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 1 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, about 9.5 mg/kg, or about 10 mg/kg.

An effective amount of the disclosed compound or its salt may be administered based on the weight, size or age of the patient. For example, a therapeutic amount may for example be in the range of about 0.01 mg/kg to about 250 mg/kg body weight, or about 0.1 mg/kg to about 10 mg/kg, in at least one dose. The patient can be administered as many doses as are required to reduce and/or alleviate and/or cure the disorder in question. When desired, formulations can be prepared with enteric coatings adapted for sustained or controlled release administration of the active ingredient.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packed tablets, capsules, and powders in vials or ampoules. Also, the unit dosage form can be a capsule, tablet, cachet, or lozenge itself, or it can be the appropriate number of any of these in packaged form.

In certain embodiments the compound is administered as a pharmaceutically acceptable salt. Non-limiting examples of pharmaceutically acceptable salts include: acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Depending on the intended mode of administration, the pharmaceutical compositions can be in the form of solid, semi-solid or liquid dosage forms, such as, for example, tablets, suppositories, pills, capsules, powders, liquids, syrup, suspensions, creams, ointments, lotions, paste, gel, spray, aerosol, foam, or oil, injection or infusion solution, a transdermal patch, a subcutaneous patch, an inhalation formulation, in a medical device, suppository, buccal, or sublingual formulation, parenteral formulation, or an ophthalmic solution, or the like, preferably in unit dosage form suitable for single administration of a precise dosage.

Some dosage forms, such as tablets and capsules, are subdivided into suitably sized unit doses containing appropriate quantities of the active components, e.g., an effective amount to achieve the desired purpose. The compositions will include an effective amount of the selected drug in combination with a pharmaceutically acceptable carrier and, in addition, can include other pharmaceutical agents, adjuvants, diluents, buffers, and the like.

Carriers include excipients and diluents and should be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to adjuvants, binders, buffering agents, coloring agents, diluents, disintegrants, excipients, emulsifiers, flavorants, gels, glidants, lubricants, preservatives, stabilizers, surfactants, solubilizer, tableting agents, wetting agents or solidifying material.

Some carriers may be listed in more than one class, for example vegetable oil may be used as a lubricant in some formulations and a diluent in others.

Exemplary pharmaceutically acceptable carriers include sugars, starches, celluloses, powdered tragacanth, malt, gelatin; talc, petroleum jelly, lanoline, polyethylene glycols, alcohols, transdermal enhancers and vegetable oils. Optional active agents may be included in a pharmaceutical composition, which do not substantially interfere with the activity of the compound of the present invention.

Some excipients include, but are not limited, to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy. Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment provided is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

In certain embodiments the excipient is selected from butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The pharmaceutical compositions/combinations can be formulated for oral administration. For oral administration, the composition may take the form of a tablet, capsule, a softgel capsule or can be an aqueous or nonaqueous solution, suspension or syrup. Tablets and capsules are typical oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

Parenteral formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing or wetting agents and suspending agents. The sterile injectable formulation can also be a sterile injectable solution or a suspension in a acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, typical methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Formulations for buccal administration include tablets, lozenges, gels and the like. Alternatively, buccal administration can be effected using a transmucosal delivery system as known to those skilled in the art. The compounds of the disclosure can also be delivered through the skin or mucosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In certain embodiments, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like.

Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound may, for example generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve.

Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

Formulations suitable for rectal administration are typically presented as unit dose suppositories. These may be prepared by admixing the active compound with one or more conventional solid carriers, for example, cocoa butter, and then shaping the resulting mixture.

In certain embodiments, the pharmaceutical composition is suitable for topical application to the skin using a mode of administration and defined above.

In certain embodiments, the pharmaceutical composition is suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound.

In certain embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

Additional non-limiting examples of drug delivery devices and methods include, for example, US20090203709 titled “Pharmaceutical Dosage Form For Oral Administration Of Tyrosine Kinase Inhibitor” (Abbott Laboratories); US20050009910 titled “Delivery of an active drug to the posterior part of the eye via subconjunctival or periocular delivery of a prodrug”, US 20130071349 titled “Biodegradable polymers for lowering intraocular pressure”, U.S. Pat. No. 8,481,069 titled “Tyrosine kinase microspheres”, U.S. Pat. No. 8,465,778 titled “Method of making tyrosine kinase microspheres”, U.S. Pat. No. 8,409,607 titled “Sustained release intraocular implants containing tyrosine kinase inhibitors and related methods”, U.S. Pat. No. 8,512,738 and US 2014/0031408 titled “Biodegradable intravitreal tyrosine kinase implants”, US 2014/0294986 titled “Microsphere Drug Delivery System for Sustained Intraocular Release”, U.S. Pat. No. 8,911,768 titled “Methods For Treating Retinopathy With Extended Therapeutic Effect” (Allergan, Inc.); U.S. Pat. No. 6,495,164 titled “Preparation of injectable suspensions having improved injectability” (Alkermes Controlled Therapeutics, Inc.); WO 2014/047439 titled “Biodegradable Microcapsules Containing Filling Material” (Akina, Inc.); WO 2010/132664 titled “Compositions And Methods For Drug Delivery” (Baxter International Inc. Baxter Healthcare SA); US20120052041 titled “Polymeric nanoparticles with enhanced drugloading and methods of use thereof” (The Brigham and Women's Hospital, Inc.); US20140178475, US20140248358, and US20140249158 titled “Therapeutic Nanoparticles Comprising a Therapeutic Agent and Methods of Making and Using Same” (BIND Therapeutics, Inc.); U.S. Pat. No. 5,869,103 titled “Polymer microparticles for drug delivery” (Danbiosyst UK Ltd.); U.S. Pat. No. 8,628,801 titled “Pegylated Nanoparticles” (Universidad de Navarra); US2014/0107025 titled “Ocular drug delivery system” (Jade Therapeutics, LLC); U.S. Pat. No. 6,287,588 titled “Agent delivering system comprised of microparticle and biodegradable gel with an improved releasing profile and methods of use thereof”, U.S. Pat. No. 6,589,549 titled “Bioactive agent delivering system comprised of microparticles within a biodegradable to improve release profiles” (Macromed, Inc.); U.S. Pat. Nos. 6,007,845 and 5,578,325 titled “Nanoparticles and microparticles of non-linear hydrophilichydrophobic multiblock copolymers” (Massachusetts Institute of Technology); US20040234611, US20080305172, US20120269894, and US20130122064 titled “Ophthalmic depot formulations for periocular or subconjunctival administration (Novartis Ag); U.S. Pat. No. 6,413,539 titled “Block polymer” (Poly-Med, Inc.); US 20070071756 titled “Delivery of an agent to ameliorate inflammation” (Peyman); US 20080166411 titled “Injectable Depot Formulations And Methods For Providing Sustained Release Of Poorly Soluble Drugs Comprising Nanoparticles” (Pfizer, Inc.); U.S. Pat. No. 6,706,289 titled “Methods and compositions for enhanced delivery of bioactive molecules” (PR Pharmaceuticals, Inc.); and U.S. Pat. No. 8,663,674 titled “Microparticle containing matrices for drug delivery” (Surmodics).

### X. Biological Data

### Biological Example 1

### CBP/P300 Degradation Protocol:

Assay media was DMEM no-phenol red medium and fetal bovine serum (FBS) were purchased from Gibco (Grand Island, N.Y., USA). Nano-Glo®, 1HiBiT Lydc Assay Systeni was purchased from Promega (Madison, Wis. USA). Stable HEK293T cell line with endogenously tagged HiBiT-CBP was generated in house by CRISPR/Cas9 gene editing introducing the HiBiT fusion tag at the N-terminus using homologous directed repair. Cell culture flasks and 384-well microplates were acquired from VWR (Radnor, Pa., USA).

CBP degradation was determined based on quantification of luminescent signal using Nano-Glo® HiBiT Lytic Assay kit. Test compounds were added to the 384-well plate from a top concentration of 10 μM concentration of 10 μM with 11 points, half log titration in duplicates, Cells were added into 384-well Corning 3570 plates, (Corning, Tewksbury, MA, USA) at a cell density of 10,000 cells per well in assay media. The plates were incubated at 37° C. with 5% CO₂ for 6 hours. The cells treated in the absence of the test compound were the negative control and media was the positive control. After 6-hour incubation, Nano-Glo® HiBiT Lytic Assay reagents were added to the cells, Luminescence was acquired on EnVision™ Multilabel Reader (PerkinElmer, Santa Clara, Calif., USA).

| | CBP/P300 | |
|---|---|---|
| | 6 h DC₅₀ | FP CRBN_ |
| | (nM) | DDB1.3 ka |
| Compound | (Emax%) | (nM) |
| 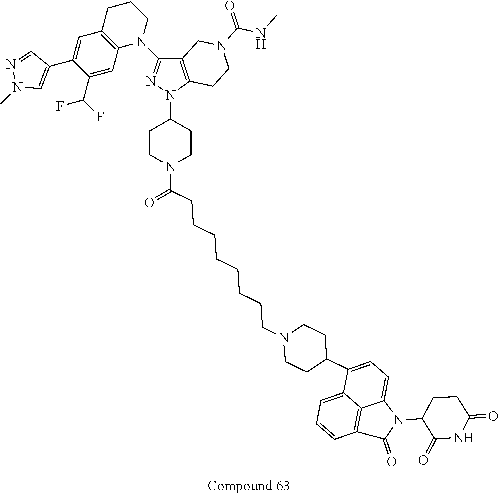 [SMILES: CNC(=O)N1CCc2c(c(N3CCCc4cc(-c5cnn(C)c5)c(C(F)F)cc43)nn2C2CCN(C(=O)CCCCCCCCN3CCC(c4ccc5c6c(cccc46)C(=O)N5C4CCC(=O)NC4=O)CC3)CC2)C1] | <10 (<30%) | <10 |
| 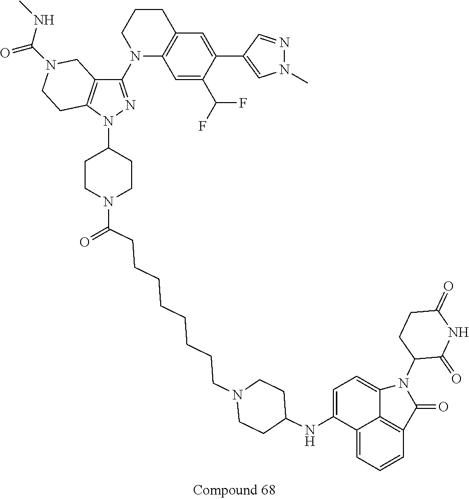 [SMILES: CNC(=O)N1CCc2c(c(N3CCCc4cc(-c5cnn(C)c5)c(C(F)F)cc43)nn2C2CCN(C(=O)CCCCCCCCN3CCC(Nc4ccc5c6c(cccc46)C(=O)N5C4CCC(=O)NC4=O)CC3)CC2)C1] | <10 (<30%) | <10 |

### Biological Example 2

### NRAS Degradation Protocol:

Assay media was DMEM no-phenol red medium and fetal bovine serum (FBS) were purchased from Gibco (Grand Island, N.Y., USA). Nano-Glo® HiBiT Lytic Assay System was purchased from Promega (Madison, Wis., USA). Stable HEK293T HiBiT-NRAS cell line was generated in house, ectopically expressing NRAS domain with HiBiT fusion tag at the C-terminus using lentivirus. Cell culture flasks and 384-well microplates were acquired from VWR. (Radnor, Pa., USA).

NR-AS degradation was determined based on quantification of luminescent signal using Nano-Glo® HiBiT Lytic Assay kit. Test compounds were added to the 384-well plate from a top concentration of 10 μM with 10 points, half log titration in duplicates. Cells were added into 384-well plates at a cell density of 2500 cells per well in assay media. In The plates were incubated at 37° C. with 5% CO2 for 24 hours. The cells treated in the absence of the test compound were the negative control and media was the positive control. After 24-hour incubation, Nano-Glo® HiBiT Lytic Assay reagents were added to the cells. Luminescence was acquired on EnVision™ Multilabel Reader (PerkinElmer, Santa Clara, Calif., USA).

### Biological Example 3: CRBN Binding Data

### NanoBRET™ Assay

The cell permeability and binding affinity of test compounds to cellular cereblon (CRBN) was determined by competitive displacement of a pomalidomide-NanoBRET™ tracer reversibly bound to a CRBN-NanoLuc® fusion protein in 293T cells. 293T cells were modified by lentiviral transfection to express a fusion of CRBN and NanoLuc® luciferase. The modified CRBN-NanoLuc 293T cell line was co-treated with varying concentrations of test compound and a pomalidomide probe conjugated with NanoBRET fluorescent tracer at its predetermined KD concentration (300 nM) and incubated for 2 hours at 37° C. to reach equilibrium. Affinity of test compound was determined by displacement of NanoBRET-pomalidomide tracer signal following the addition of NanoBRET reagents (Promega) per manufacturer's instructions.

40 uL 293T cells suspended in OptiMEM media at 2×105 cells/mL (8000 cells/well) were dispensed using a Multidrop Combi Reagent Dispenser (Thermo Fisher) to each well of 384-well white TC-treated microplates. 10 mM DMSO test compound stock solution was serially diluted (half log) in DMSO to generate 11-point dose series (10000, 3160, 1000, 316, 100, 31.6, 10, 3.2, 1, 0.3, 0.1 μM) in an acoustic ready 384-well low dead volume microplate (Labcyte). Using Echo 550 Acoustic Liquid Handler (Labcyte), 40 nL of serially diluted compound solutions were dispensed in duplicate to each 384-well white TC-treated microplate containing 293T cells. 40 nL DMSO was transferred to all control wells. 40 nL NanoBRET-pomalidomide tracer was dispensed to all wells in column 1-23. 40 nL additional DMSO was dispensed to column 24. Final concentration of DMSO was 0.2% for all samples. Plates were spun briefly and cells were incubated at 37° C.; 5% CO2 for 2 hr. 20 uL NanoBRET TE Assay reagents were added to each well and NanoBRET signal was acquired on an EnVision Multilabel Reader (PerkinElmer). Donor emission from CRBN-NanoLuc was detected at 450 nm with a NanoLuc 460/50 filter and Acceptor fluorescence of NanoBRET-pomalidomide tracer (618 nm) was detected with a 600 nm long pass NanoBRET filter. Ratio of Acceptor signal/Donor signal was calculated for each well. Column 24 (cells without NanoBRET-pomalidomide tracer addition) was used as positive control (P).

Percent response of compound-treated samples (T) were calculated by normalizing the Acceptor/Donor ratio for each well to the DMSO treated negative (N) controls on the same microtiter plate after background (i.e. positive control) signal subtraction: Response %=100×(Signal(T)−Average (P))/(Average (N)−Average (P)).

### CRBN FP Binding Assay

The determination of the binding constant (KD) of test compounds to CRBN-DDB1 was carried out using an established sensitive and quantitative in vitro fluorescence polarization (FP) binding assay. Control compounds were run on the same plate. Compounds were dispensed from serially diluted DMSO stock supplied by Frontier Scientific Services Inc in low dead volume plates into black 384-well compatible FP plates using acoustic technology to 1% of total reaction volume. Compounds were arranged vertically in rows A through P. Concentrations series are horizontal: columns 1-11, and then duplicates in columns 12-22. Columns 23 and 24 are reserved for 0% (5 nM probe) and 100% controls (protein at high concentration with 5 nM probe), respectively. Compound binding to CRBN-DDB1 was measured by displacement of Alexa-647 Fluor® based probe with a KD of 113 nM, as determined by a single site ligand depletion model. A 20 μL mixture containing 150 nM CRBN-DDB1 and 5 nM probe dye in 50 mM HEPES, pH 7.4, 200 mM NaCl, 1 mM TCEP and 0.05% pluronic acid-127 was added to wells containing compound and incubated at room temperature for 1.5 hours. Controls wells with 100% bound probe contained 1500 nM of CRBN. Matching control plates excluding CRBN-DDB1 were used to correct for background fluorescence. Plates were read on an Envision plate reader with appropriate FP filter sets.

| | | | NanoBRET |
|---|---|---|---|
| | FP | NanoBRET | 293T |
| | CRBN_DDB1.3 | 293T 2 h | CRBN 2 h |
| Structure | ka (nM) | IC50 (nM) | IC50 (nM) |
| 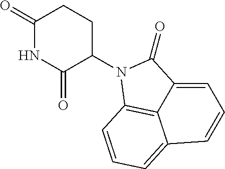 [SMILES: O=C1CCC(N2C(=O)c3cccc4cccc2c34)C(=O)N1] | + | + | + |
| 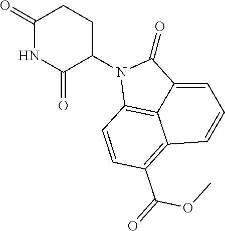 [SMILES: COC(=O)c1ccc2c3c(cccc13)C(=O)N2C1CCC(=O)NC1=O] | ++ | | + |
| 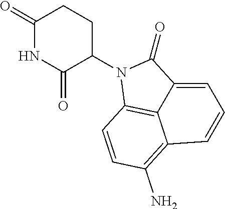 [SMILES: Nc1ccc2c3c(cccc13)C(=O)N2C1CCC(=O)NC1=O] | ++ | | + |
| 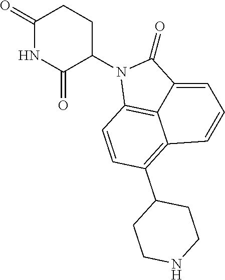 [SMILES: O=C1CCC(N2C(=O)c3cccc4c(C5CCNCC5)ccc2c34)C(=O)N1] | | | + |
| 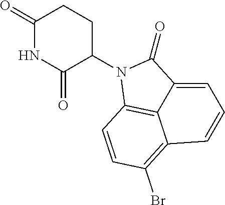 [SMILES: O=C1CCC(N2C(=O)c3cccc4c(Br)ccc2c34)C(=O)N1] | | + | |
| 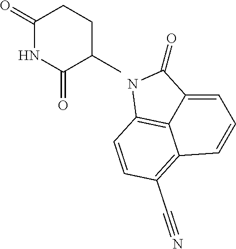 | | + | |

+ = <100 nM;
++ = 100-1000 nM

### Biological Example 4: ERK HTRF Method

### Materials

Colo205 CCL-222 cells were purchased from ATCC. RPMI 1640 no-phenol red medium and fetal bovine serum (FBS) were purchased from Gibco (Grand Island, NY, USA). Cell culture flasks and 384-well microplates were acquired from VWR (Radnor, PA, USA). Total ERK HTRF assay kits were purchased from Cisbio (64NRKPEH, Bedford, MA, USA).

### ERK1/2 Degradation Analysis

Degradation of ERK1/2 was determined based on quantification of FRET signal using a Total ERK1/2 HTRF assay kit. Test compounds were added to the 384-well plate from a top concentration of 10 μM with 10 points, half-log titration in duplicates. COL0205 cells were added into 384-well plates at a cell density of 5,000 cells per well. The plates were kept at 37° C. with 5% CO2 for 6 hours. Cells treated in the absence of the test compound were the negative control. Positive control was set by wells containing all reagents but no cells. HTRF regents were added according to manufacturer's instructions with the additional step of lysate denaturation at 95° C. for 10 minutes and cooling to room temperature before antibody addition. Following addition of the antibodies the samples were incubated overnight. FRET signal was acquired on an EnVision™ Multilabel Reader (PerkinElmer, Santa Clara, CA, USA).

| | | FP |
|---|---|---|
| | ERK1/2 | CRBN_ |
| | 6 h DC₅₀ | DDB1.3 |
| Compound | (Emax %) | ka (nM) |
| 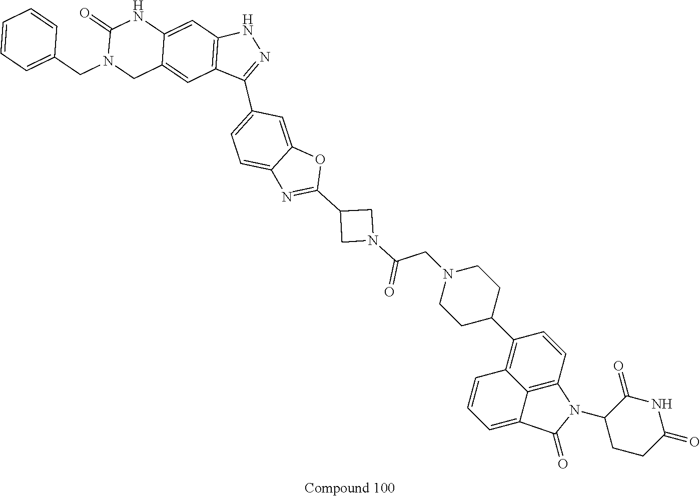 [SMILES: O=C1CCC(N2C(=O)c3cccc4c(C5CCN(CC(=O)N6CC(c7nc8ccc(-c9n[nH]c%10cc%11c(cc9%10)CN(Cc9ccccc9)C(=O)N%11)cc8o7)C6)CC5)ccc2c34)C(=O)N1] | +++ | ++ |
| 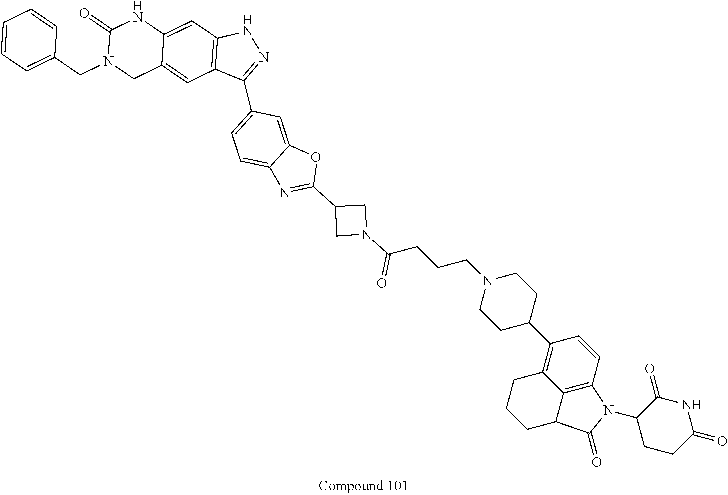 [SMILES: O=C1CCC(N2C(=O)C3CCCc4c(C5CCN(CCCC(=O)N6CC(c7nc8ccc(-c9n[nH]c%10cc%11c(cc9%10)CN(Cc9ccccc9)C(=O)N%11)cc8o7)C6)CC5)ccc2c43)C(=O)N1] | +++ | ++ |

As used in the table above for DC₅₀ values <100 nM = ++++,
100-1,000 nM = +++,
1,001-10,000 nM = ++,
>10,000 nM = +
For Emax values <45% = ++++,
45-60% = +++,
61-95% = ++,
>95% = +

### Biological Example 5: NSP3 UBL2_PLpro-HA-HiBiT Degradation Assay

### Generation of 239T Cell Lines Stably Expressing NSP3 Ubl-2_PLpro-HA-HiBiT

For quantitative cellular degradation of the target protein degradation mediated by the bifunctional degraders described here, HA and HiBiT was appended to the C-terminus of the human codon optimized gene sequence of amino acids 1564-1878 of ORF1a polyprotein from Severe acute respiratory syndrome Coronavirus 2 (Sars-CoV-2) and an NSP3 UBL2 PLpro-HA-HiBiT expressing 293T (ATCC®, CRL-3216) cell line was generated in house. Expression of the NSP3 UBL2_PLpro-HA-HiBiT was confirmed with an HA antibody at the expected molecular weight.

A NSP3 UBL2_PLpro-HA-HiBiT 293T CRBN−/− cell line was generated using a CRISPR/Cas9 edited CRBN−/− 293T cell line in a similar way.

### Materials

NSP3 UBL2_PLpro-HA-HiBiT 293T line were generated in house as described herein. The parental 293T cell line, as well as NSP3 UBL2_PLpro-HA-HiBiT 293T cell lines were routinely cultured in the following medium: DMEM (Thermo Fisher, 11965092) containing 10% serum (Thermo Fisher, 10437036) and to no more than 20 passages. For the assay, NSP3 UBL2_PLpro-HA-HiBiT cells were plated for treatment in DMEM no-Phenol Red (Thermo Fisher, 21063045 or alternative no phenol red DMEM) containing 10% serum (Thermo Fisher, 10437036). Assay were performed in Corning® 384 Well Low Flange Black Flat Bottom Polystyrene TC-Treated Microplates (Corning, 3571). Cells were lysed in Nano-Glo® HiBiT Lytic Assay System (Promega, N3050).

### NSP3 UBL2_PLpro-HA-HiBiT Degradation Assay (Cellular)

Test compounds were added to the 384-well plate from a top concentration of 10 mM with 11 points, half-log titration in duplicates and stored at −20° C. until use. Briefly, on the day of compound treatment, cells were seeded onto 384-well plate containing test compounds at the density of 2,500 cell per well in a volume resulting in the top dose of the test compounds as 10 uM. Additionally, the negative control cells were treated with vehicle alone. The plates were incubated at 37° C. with 5% CO2 for duration of the assay (6 or 24 hours). After the desired incubation time, cells were lysed by addition of Nano-Glo® HiBiT Lytic Assay System (prepared according to the manufacture recommendations and added to the cells in ratio 1:1, v/v). Microplates were agitated an orbital plate shaker at 300-600 rpm for 10 minutes and incubated for another 60 min in at room temperature. Luminescence was acquired on EnVision™ Multilabel Reader (PerkinElmer, Santa Clara, Calif., USA).

Quantification of luminescence responses measured in the presence of compound were normalized to a high signal/no degradation control (untreated cells+lytic detection reagent) and a low signal/full degradation control (untreated cells, no lytic detection reagent). Data were analyzed with a 4-parameter logistic fit to generate sigmoidal dose-response curves. The DC50 is the concentration of compound at which exactly 5000 of the total cellular NSP3 UBL2_PLpro-HA-HiBiT has been degraded. The Emax, or maximum effect of each compound, represents the amount of residual protein remaining in the cell following compound treatment.

| | | HiBiT- | |
|---|---|---|---|
| | | Degrada- | HiBiT- |
| | | tion | Degrada- |
| | | 293T.234 | tion |
| | | NSP3_P | 293T.234 |
| | | Lpro 6.0 | NSP3_P |
| | | hours | Lpro 6.0 |
| Cmpd | | (DC50) | hours |
| # | Structure | [nM] | (Emax %) |
| 102 | 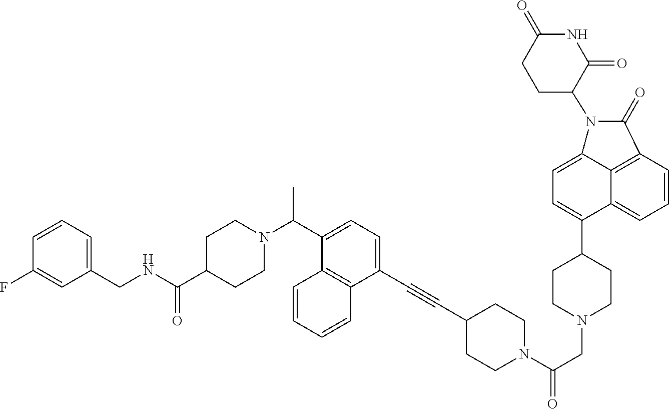 [SMILES: CC(c1ccc(C#CC2CCN(C(=O)CN3CCC(c4ccc5c6c(cccc46)C(=O)N5C4CCC(=O)NC4=O)CC3)CC2)c2ccccc12)N1CCC(C(=O)NCc2cccc(F)c2)CC1] | + | ++ |
| 103 | 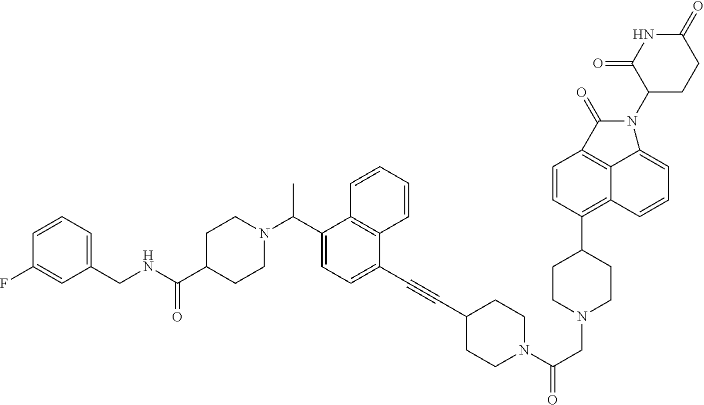 [SMILES: CC(c1ccc(C#CC2CCN(C(=O)CN3CCC(c4ccc5c6c(cccc46)N(C4CCC(=O)NC4=O)C5=O)CC3)CC2)c2ccccc12)N1CCC(C(=O)NCc2cccc(F)c2)CC1] | + | ++ |
| 104 | 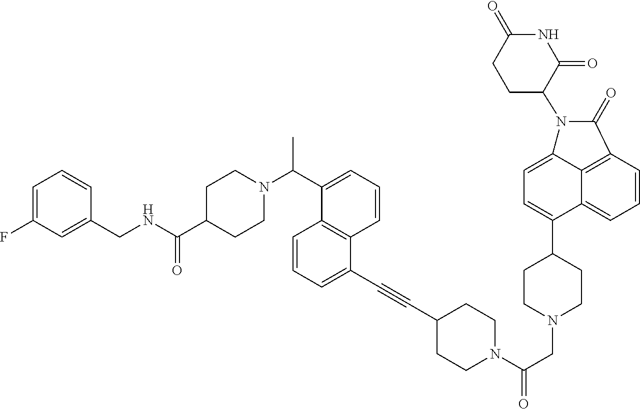 [SMILES: CC(c1cccc2c(C#CC3CCN(C(=O)CN4CCC(c5ccc6c7c(cccc57)C(=O)N6C5CCC(=O)NC5=O)CC4)CC3)cccc12)N1CCC(C(=O)NCc2cccc(F)c2)CC1] | + | ++ |
| 105 | 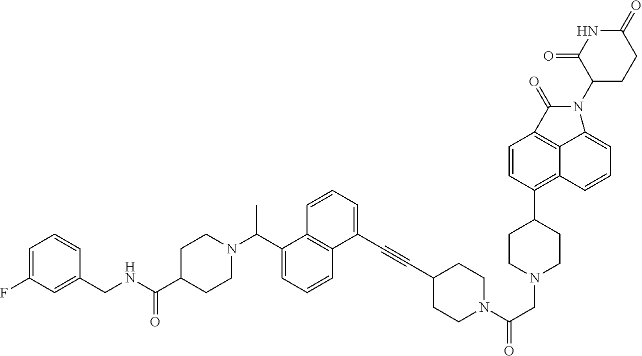 [SMILES: CC(c1cccc2c(C#CC3CCN(C(=O)CN4CCC(c5ccc6c7c(cccc57)N(C5CCC(=O)NC5=O)C6=O)CC4)CC3)cccc12)N1CCC(C(=O)NCc2cccc(F)c2)CC1] | + | ++ |

As used in the table above for DC₅₀ values <100 nM = ++++,
100-1,000 nM = +++,
1,001-10,000 nM = ++,
>10,000 nM = +
For Emax values <45% = ++++,
45-60% = +++,
61-95% = ++,
>95% = +

### XI. General Synthesis

The compounds described herein can be prepared by methods known by those skilled in the art. As non-limiting examples, the disclosed compounds can be made using the schemes below.

Compounds of the present invention with stereocenters may be drawn without stereochemistry for convenience. One skilled in the art will recognize that pure or enriched enantiomers and diastereomers can be prepared by methods known in the art. Examples of methods to obtain optically active materials include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the enantiomer is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step in the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e. chirality) in the product, which may be achieved by chiral catalysts or chiral auxiliaries;
vi) diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer;
vii) first- and second-order asymmetric transformations—a technique whereby diastereomers from the racemate quickly equilibrate to yield a preponderance in solution of the diastereomer from the desired enantiomer of where preferential crystallization of the diastereomer from the desired enantiomer perturbs the equilibrium such that eventually in principle all the material is converted to the crystalline diastereomer from the desired enantiomers. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase (including vial chiral HPLC). The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is place in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through;
xiv) simulated moving bed chromatography is used in certain embodiments. A wide variety of chiral stationary phases are commercially available.

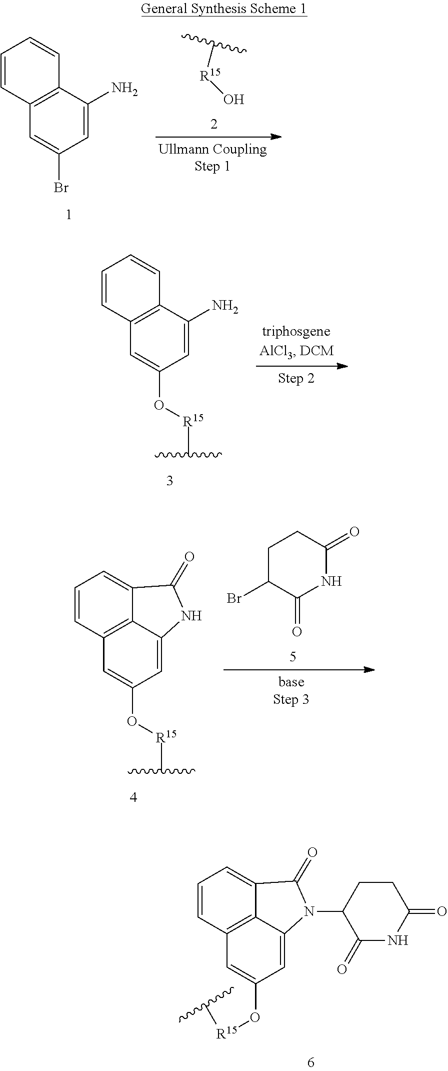

A compound of Formula III can be synthesized according to the route provided in General Synthesis Scheme 1. In step 1, compound 1 is reacted with 2 in the presence of a copper catalyst (for example, copper(I) iodide, copper(I) chloride, or alternatively another suitable copper catalyst used in Ullmann coupling conditions), a ligand (for example, bipyridine, 1,10-phenanthroline, dimethylethylenediamine, or alternatively another suitable ligand used in Ullmann coupling conditions), and a base (for example, cesium carbonate, potassium carbonate, tribasic potassium phosphate, or alternatively another suitable base used in Ullmann coupling conditions) in organic solvent (for example, dimethylsulfoxide, acetonitrile, or dioxane) at elevated temperature to afford 3. In step 2, compound 3 is reacted with triphosgene in the presence of aluminum trichloride in dichloromethane to afford 4. In step 3, compound 4 is reacted with a base (for example, sodium hydride) in an organic solvent (for example, tetrahydrofuran or dichloromethane) followed by the addition of 5 to afford 6.

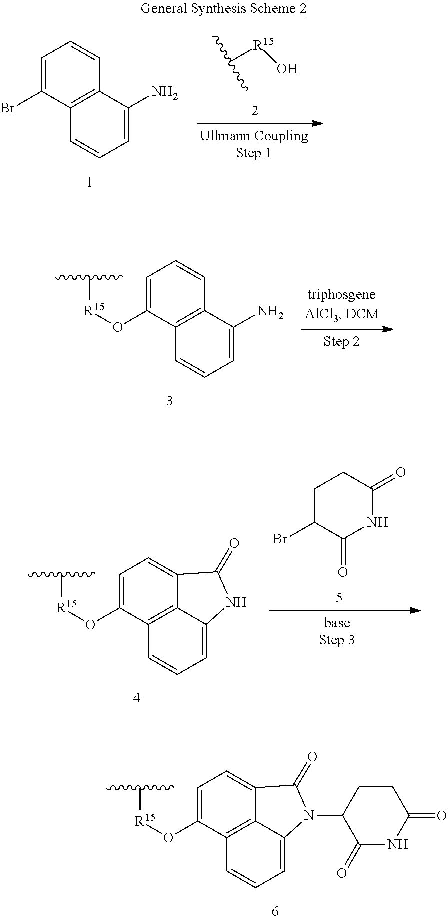

A compound of Formula III can be synthesized according to the route provided in General Synthesis Scheme 2. In step 1, compound 1 is reacted with 2 in the presence of a copper catalyst (for example, copper(I) iodide, copper(I) chloride, or alternatively another suitable copper catalyst used in μLlmann coupling conditions), a ligand (for example, bipyridine, 1,10-phenanthroline, dimethylethylenediamine, or alternatively another suitable ligand used in μLlmann coupling conditions), and a base (for example, cesium carbonate, potassium carbonate, tribasic potassium phosphate, or alternatively another suitable base used in μLlmann coupling conditions) in organic solvent (for example, dimethylsulfoxide, acetonitrile, or dioxane) at elevated temperature to afford 3. In step 2, compound 3 is reacted with triphosgene in the presence of aluminum trichloride in dichloromethane to afford 4. In step 3, compound 4 is reacted with a base (for example, sodium hydride) in an organic solvent (for example, tetrahydrofuran or dichloromethane) followed by the addition of 5 to afford 6.

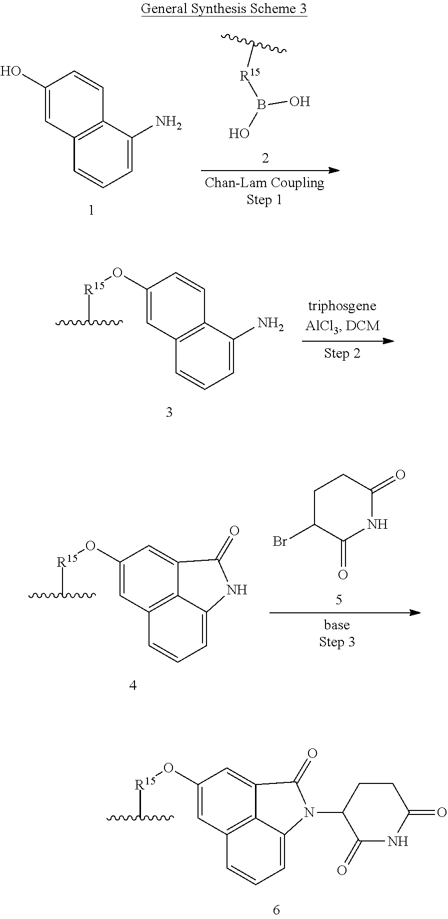

A compound of Formula III can be synthesized according to the route provided in General Synthesis Scheme 3. In step 1, compound 1 is reacted with 2 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 3. In step 2, compound 3 is reacted with triphosgene in the presence of aluminum trichloride in dichloromethane to afford 4. In step 3, compound 4 is reacted with a base (for example, sodium hydride) in an organic solvent (for example, tetrahydrofuran or dichloromethane) followed by the addition of 5 to afford 6.

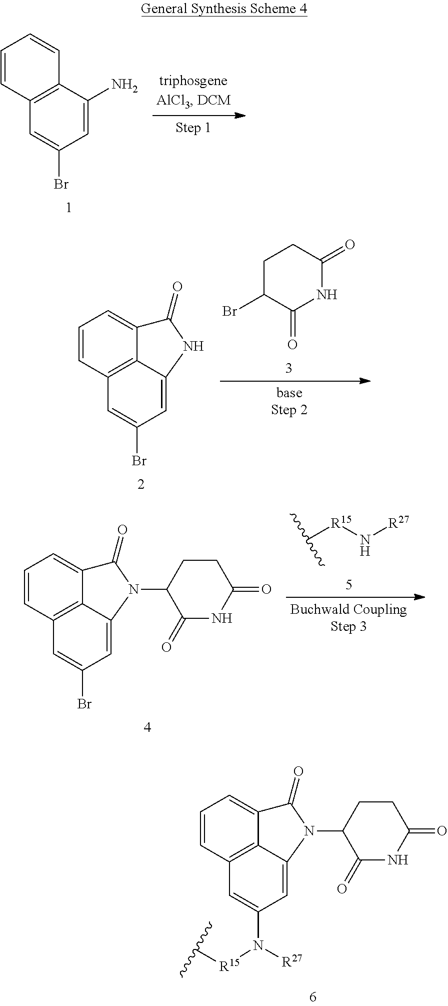

A compound of Formula III can be synthesized according to the route provided in General Synthesis Scheme 4. In step 1, compound 1 is reacted with triphosgene in the presence of aluminum trichloride in dichloromethane to afford 2. In step 2, compound 2 is reacted with a base (for example, sodium hydride) in an organic solvent (for example, tetrahydrofuran or dichloromethane) followed by the addition of 3 to afford 4. In step 3, compound 4 is reacted with in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 6.

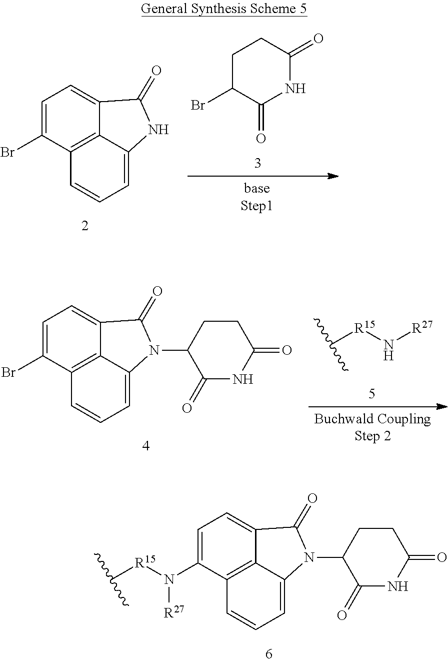

A compound of Formula III can be synthesized according to the route provided in General Synthesis Scheme 5. In step 1, compound 2 is reacted with a base (for example, sodium hydride) in an organic solvent (for example, tetrahydrofuran or dichloromethane) followed by the addition of 3 to afford 4. In step 2, compound 4 is reacted with 5 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 6.

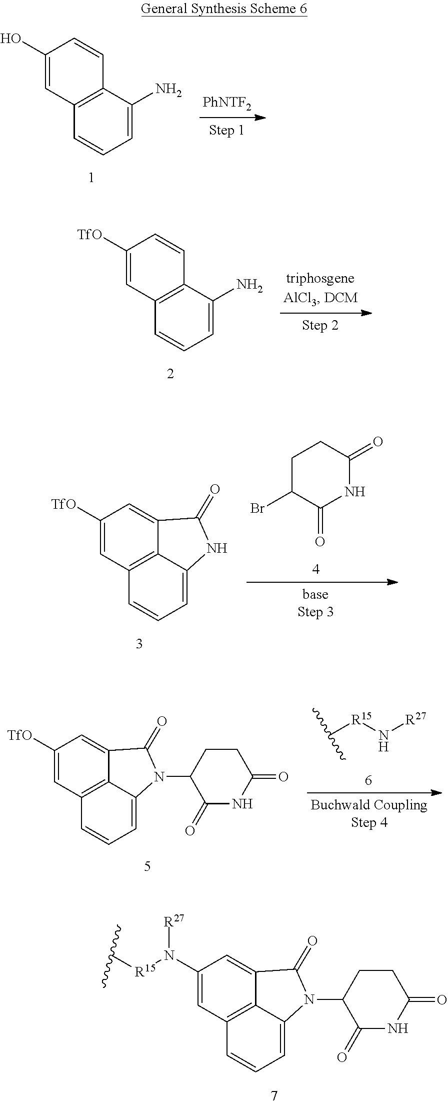

A compound of Formula III can be synthesized according to the route provided in General Synthesis Scheme 6. In step 1, compound 1 is reacted with phenyl triflimide in the presence of a base (for example, pyridine, triethylamine, or alternatively another suitable base used in triflating conditions) in organic solvent (for example, dichloromethane or toluene) to afford 2. In step 2, compound 2 is reacted with triphosgene in the presence of aluminum trichloride in dichloromethane to afford 3. In step 3, compound 3 is reacted with a base (for example, sodium hydride) in an organic solvent (for example, tetrahydrofuran or dichloromethane) followed by the addition of 4 to afford 5. In step 4, compound 5 is reacted with 6 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 7.

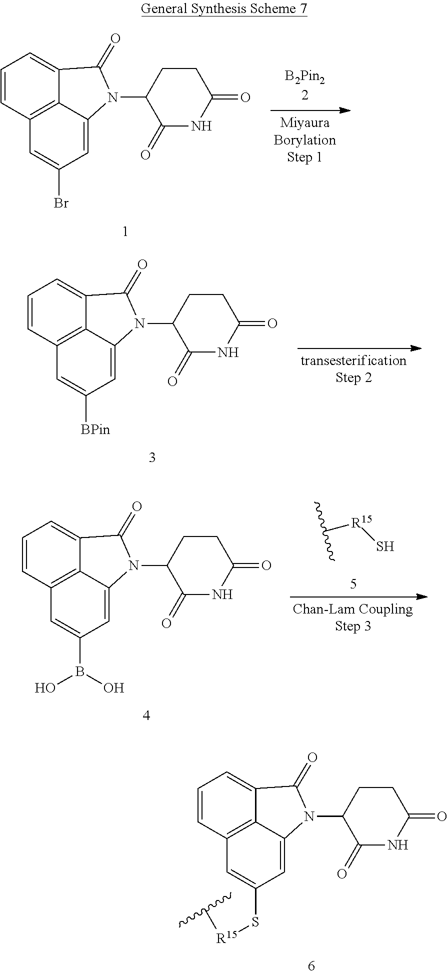

A compound of Formula III can be synthesized according to the route provided in General Synthesis Scheme 7. In step 1, compound 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, compound 3 is subjected to transesterification to afford 4. In step 3, compound 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

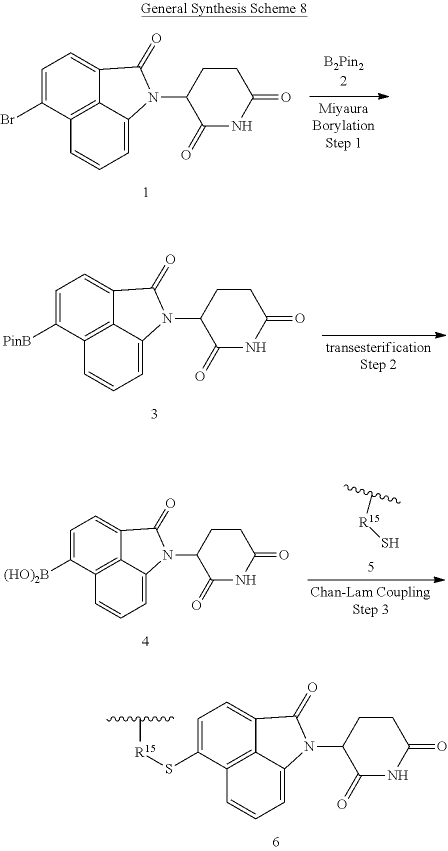

A compound of Formula III can be synthesized according to the route provided in General Synthesis Scheme 8. In step 1, compound 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, compound 3 is subjected to transesterification to afford 4. In step 3, compound 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

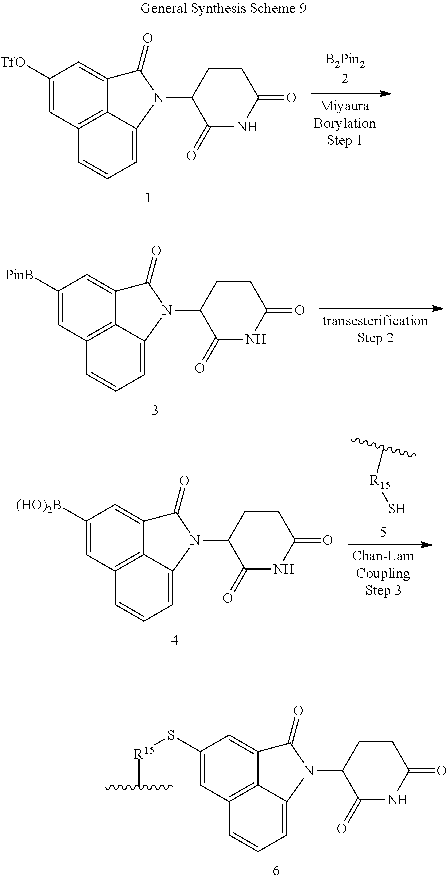

A compound of Formula III can be synthesized according to the route provided in General Synthesis Scheme 9. In step 1, compound 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, compound 3 is subjected to transesterification to afford 4. In step 3, compound 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

General Synthesis Scheme 10

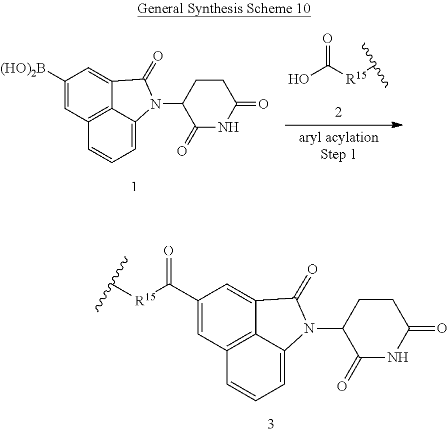

A compound of Formula III can be synthesized according to the route provided in General Synthesis Scheme 10. In step 1, compound 1 is reacted with 2 in the presence of a palladium catalyst (for example, Pd(OAc)₂, Pd(PPh₃)₄, or alternatively another suitable palladium catalyst), a ligand (for example, P(p-MeOPh)₃, PPh₃, PCy₃ or alternatively another suitable ligand), water, and pivalic anhydride in organic solvent (for example, dimethoxyethane, THF, or toluene) at elevated temperature to afford 3.

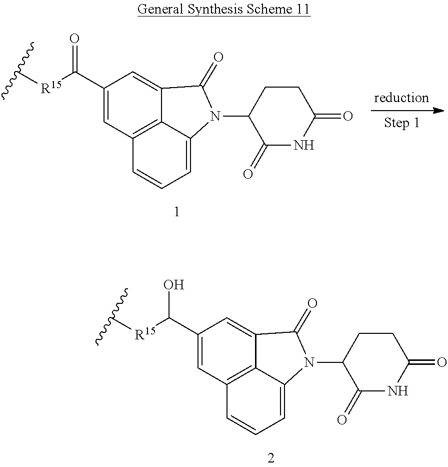

A compound of Formula III can be synthesized according to the route provided in General Synthesis Scheme 11. In step 1, compound 1 is reacted with a suitable carbonyl reductant (for example, sodium borohydride) in organic solvent (for example, ethanol or methanol) to afford 2.

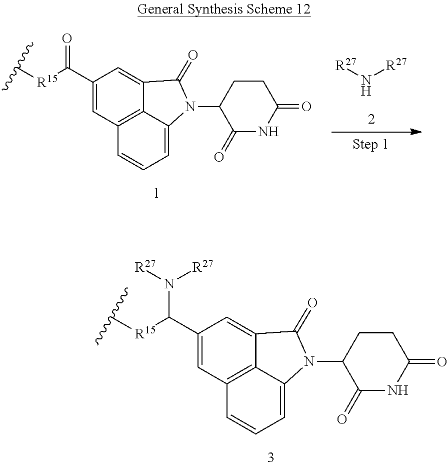

A compound of Formula III can be synthesized according to the route provided in General Synthesis Scheme 12. In step 1, compound 1 is reacted with 2 in the presence of a suitable reductant (for example, sodium triacetoxyborohydride or sodium cyanoborohydride) in organic solvent (for example, methanol or ethanol) to afford 2.

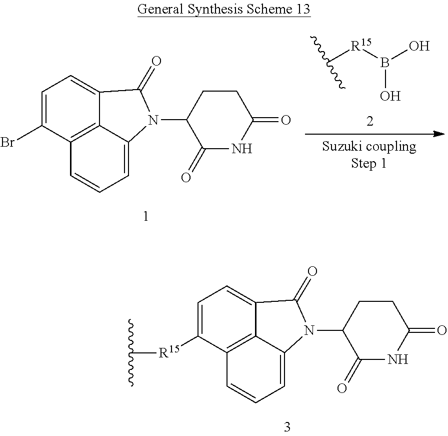

A compound of Formula III can be synthesized according to the route provided in General Synthesis Scheme 13. In step 1, compound 1 is reacted with 2 in the presence of a palladium catalyst (for example, Pd(OAc)₂, Pd₂dba₃, or alternatively another suitable palladium catalyst used in Suzuki coupling conditions), a ligand (for example, XPhos, PCy₃, or alternatively another suitable ligand used in Suzuki coupling conditions), and a base (for example, sodium carbonate, tribasic potassium phosphate, potassium carbonate, or alternatively another suitable base used in Suzuki coupling conditions) in aqueous organic solvent (for example, 10:1 toluene:water, 5:1 THF:water, or 1:1 ethanol:water) at elevated temperature to afford 3.

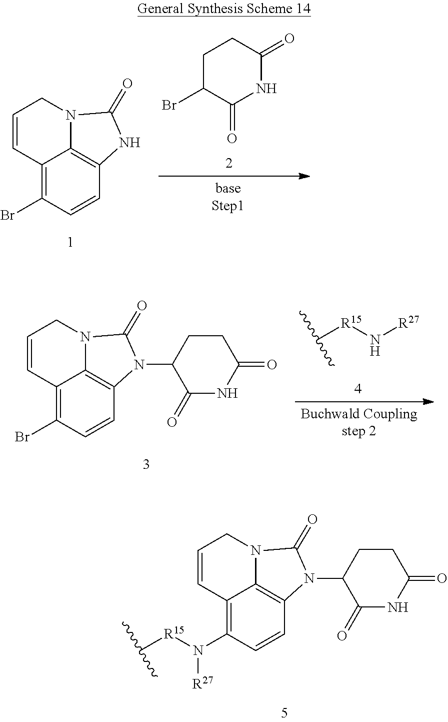

A compound of Formula I can be synthesized according to the route provided in General Synthesis Scheme 14. In step 1, intermediate 1 (prepared by the procedure of Saari et al. see: Saari, W. et al. “Synthesis and reactions of some dihydro and tetrahydro-4H-imidazo[5,4,1-ij]quinoline derivatives” Journal of Heterocyclic Chemistry, 1982, 19(4):837-840) is reacted with a base (for example, sodium hydride) in an organic solvent (for example, tetrahydrofuran or dichloromethane) followed by the addition of 2 to afford 3. In step 2, 3 is reacted with 4 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 5.

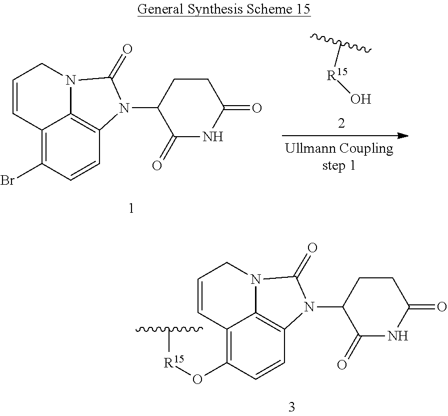

A compound of Formula I can be synthesized according to the route provided in General Synthesis Scheme 15. In step 1, intermediate 1 is reacted with 2 in the presence of a copper catalyst (for example, copper(I) iodide, copper(I) chloride, or alternatively another suitable copper catalyst used in μLlmann coupling conditions), a ligand (for example, bipyridine, 1,10-phenanthroline, dimethylethylenediamine, or alternatively another suitable ligand used in μLlmann coupling conditions), and a base (for example, cesium carbonate carbonate, tribasic potassium phosphate, or alternatively another suitable base used in μLlmann coupling conditions) in organic solvent (for example, dimethylsulfoxide, acetonitrile, or dioxane) at elevated temperature to afford 3.

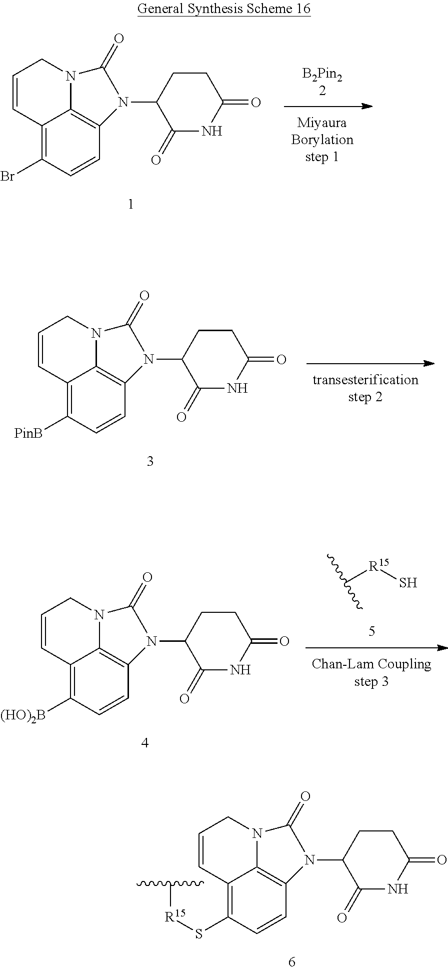

A compound of Formula I can be synthesized according to the route provided in General Synthesis Scheme 16. In step 1, 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, intermediate 3 is transesterification to afford 4. In step 3, intermediate 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6.

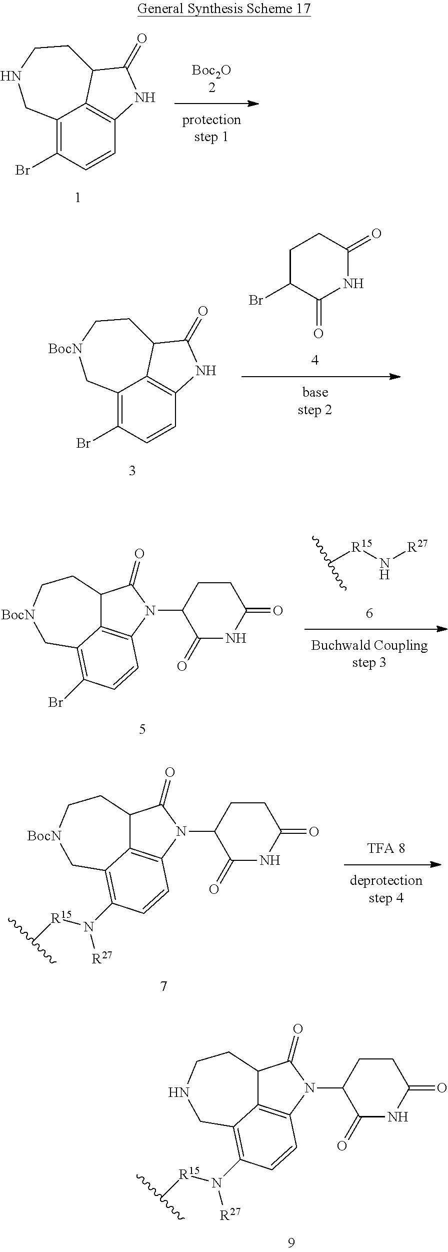

A compound of Formula I can be synthesized according to the route provided in General Synthesis Scheme 17. In step 1, intermediate 1 (prepared by the procedure of Kukla et al. see: Kukla, M. J. et al. “Synthesis and anti-HIV-1 activity of 4,5,6,7-tetrahydro-5-methylimidazo[4,5,1-jk][1,4]benzodiazepin-2(1H)-one (TIBO) derivatives” J. Med. Chem. 1991, 34(11):3187-3197) is reacted with 2 in the presence of a base (for example triethylamine, pyridine, or other suitable base used in Boc protection conditions) in dichloromethane to provide 3. In step 2, intermediate 3 is reacted with a base (for example sodium hydride) in an organic solvent (for example tetrahydrofuran or dichloromethane) followed by addition of 4 to provide 5. In step 3, intermediate 5 is reacted with 6 in the presence of a palladium catalyst (for example palladium(II) acetate, Pd₂(dba)₃, or other suitable palladium catalyst used in Buchwald-Hartwig coupling conditions), a phosphine ligand (for example BINAP, XantPhos, or other suitable phosphine ligand used in Buchwald-Hartwig coupling conditions), and a base (for example potassium tert-butoxide, cesium carbonate, or other suitable base used in Buchwald-Hartwig coupling conditions) in organic solvent (for example toluene, THF, dioxane, or DMF) at elevated temperature to provide 7. In step 4, intermediate 7 is reacted with 8 in dichloromethane to provide 9.

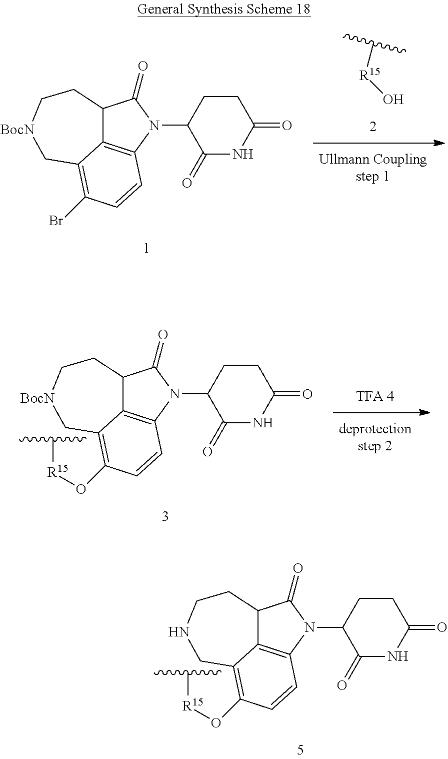

A compound of Formula I can be synthesized according to the route provided in General Synthesis Scheme 18. In step 1, intermediate 1 is reacted with 2 in the presence of a copper catalyst (for example, copper(I) iodide, copper(I) chloride, or alternatively another suitable copper catalyst used in μLlmann coupling conditions), a ligand (for example, bipyridine, 1,10-phenanthroline, dimethylethylenediamine, or alternatively another suitable ligand used in μLlmann coupling conditions), and a base (for example, cesium carbonate, potassium carbonate, tribasic potassium phosphate, or alternatively another suitable base used in μLlmann coupling conditions) in organic solvent (for example, dimethylsulfoxide, acetonitrile, or dioxane) at elevated temperature to afford 3. In step 2, intermediate 3 is reacted with 4 in dichloromethane to afford 5.

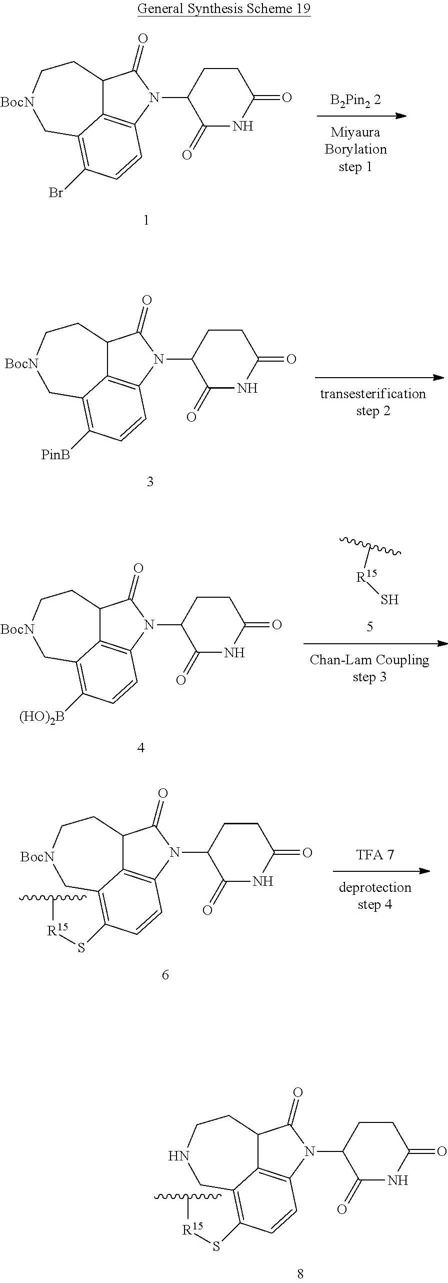

A compound of Formula I can be synthesized according to the route provided in General Synthesis Scheme 19. In step 1, 1 is reacted with 2 in the presence of a palladium catalyst (for example, PdCl₂(dppf), PdCl₂(PPh₃), or alternatively another suitable palladium catalyst used in Miyaura coupling conditions), a ligand (for example, XPhos, PPh₃, or alternatively another suitable ligand used in Miyaura coupling conditions), and a base (for example, potassium acetate, potassium ethoxide, potassium carbonate, or alternatively another suitable base used in Miyaura coupling conditions) in organic solvent (for example, toluene, DMA, or dioxane) at elevated temperature to afford 3. In step 2, intermediate 3 is subjected to transesterification to afford 4. In step 3, intermediate 4 is reacted with 5 in the presence of a copper catalyst (for example, copper(II) bromide, copper(II) acetate, or alternatively another suitable copper catalyst used in Chan-Lam coupling conditions) and a base (for example, pyridine, 4-dimethylaminopyridine, potassium tert-butoxide, or alternatively another suitable base used in Chan-Lam coupling conditions) in organic solvent (for example, methanol, acetonitrile, or dichloromethane) under ambient air to afford 6. In step 4, 6 is reacted with 7 in dichloromethane to afford 8.

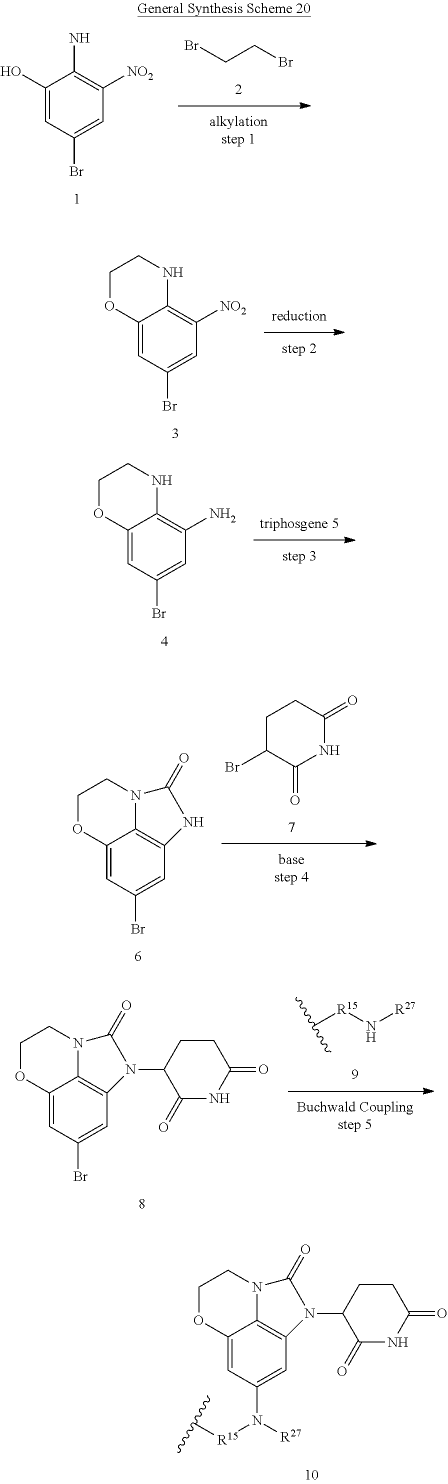

A compound of Formula I can be synthesized according to the route provided in General Synthesis Scheme 20. In step 1, intermediate 1 is reacted with 2 in the presence of base (for example potassium carbonate, cesium carbonate, or other suitable base used in phenol alkylation conditions) in organic solvent (for example DMF, DMA, or acetonitrile) at elevated temperature to provide 3. In step 2, intermediate 3 is reacted iron powder with HCl under aqueous conditions temperature to provide 4. In step 3, 4 is reacted with triphosgene in the presence of aluminum trichloride in dichloromethane to afford 6. In step 4, intermediate 6 is reacted with a base (for example sodium hydride) in an organic solvent (for example tetrahydrofuran or dichloromethane) followed by addition of 7 to provide 8. In step 5, 8 is reacted with 9 in the presence of a palladium catalyst (for example, palladium(II) acetate, Pd₂(dba)₃, or alternatively another suitable palladium catalyst used in Buchwald-Hartwig coupling conditions), a phosphine ligand (for example, BINAP, XantPhos, or alternatively another suitable phosphine ligand used in Buchwald-Hartwig coupling conditions), and a base (for example, potassium tert-butoxide, cesium carbonate, or alternatively another suitable base used in Buchwald-Hartwig coupling conditions) in organic solvent (for example, toluene, THF, dioxane, or DMF) at elevated temperature to afford 10.

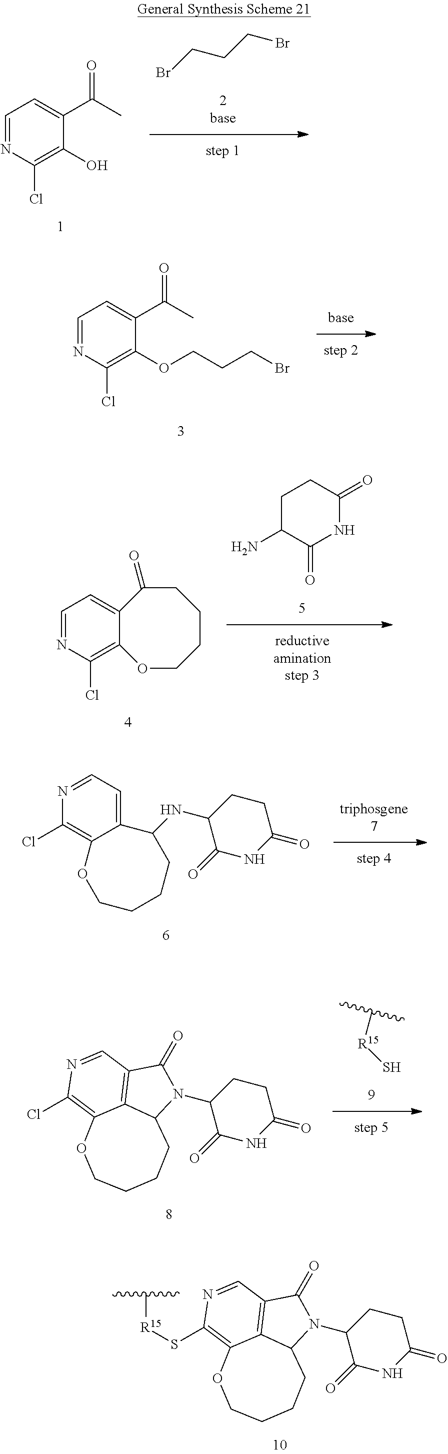

A compound of Formula I can be synthesized according to the route provided in General Synthesis Scheme 21. In step 1, intermediate 1 is reacted with 2 in the presence of base (for example potassium carbonate, cesium carbonate, or other suitable base used in phenol alkylation conditions) in organic solvent (for example DMF, DMA, or acetonitrile) at elevated temperature to provide 3. In step 2, 3 is reacted base (for example, LDA, LiHMDS, or other suitable strong, sterically hindered base). In step 3, 4 is reacted with 5 in the presence of a mild reductant (for example, sodium triacetoxyborohydride, sodium cyanoborohydride, or other suitable hydride reductant used in reductive amination conditions) in organic solvent (for example methanol, acetonitrile, or dichloromethane) to provide 6. In step 4, 6 is reacted with triphosgene in the presence of aluminum trichloride in dichloromethane to afford 8. In step 5, 8 is reacted with 9 in organic solvent (for example DMF, DMA, or dioxane) at elevated temperature to afford 10.

### Example 1. Synthesis of tert-butyl N-[[1-(2,6-dioxo-3-piperidyl)-2-oxo-benzo[cd]indol-6-yl]methyl]carbamate (Compound 1)

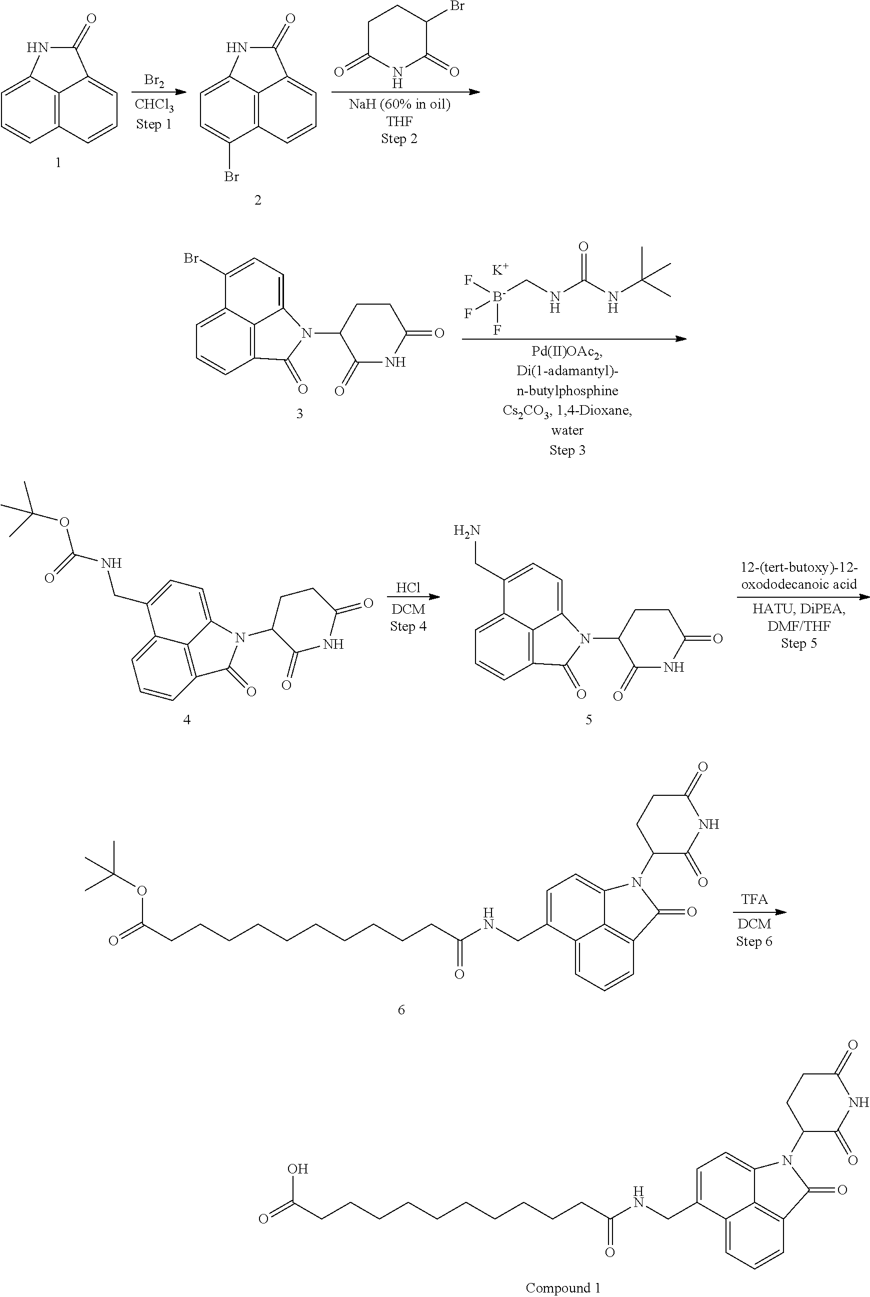

Step 1: To a solution of 1H-benzo[cd]indol-2-one 1 (10 g, 59.11 mmol) in chloroform (100 mL) at 0° C. was added a solution of molecular bromine (14.17 g, 88.66 mmol, 4.54 mL) in chloroform (20 mL). The resulting mixture stirred at RT overnight. At this time, the reaction was poured into sat. aq. sodium thiosulfate. The yellow solid that formed was isolated by vacuum filtration, washed with water and pentane, and put on the lyophilizer to afford 6-bromo-1H-benzo[cd]indol-2-one 2 (14.59 g, 49.99 mmol, 84.57% yield, 85% purity), which was used without further purification.

Step 2: To a cooled solution of 6-bromo-1H-benzo[cd]indol-2-one 2 (40 g, 161.24 mmol) in dry THE (2 mL), sodium hydride (60% dispersion in mineral oil) (37.07 g, 1.61 mol) was added in portions, maintaining the temp at ˜ 5° C. Once the addition was complete, the resultant mixture was stirred for 30 minutes at room temperature. Then the reaction mixture was again cooled to 0° C. and a solution of 3-bromopiperidine-2,6-dione (154.80 g, 806.21 mmol) was added dropwise. After completion of the addition, the resulting solution was heated at 65° C. 16 hr. The reaction mixture was then cooled to 0° C., quenched with the addition of ice cooled aqueous ammonium chloride solution and extracted with ethyl acetate. Combined extract was dried over anhydrous sodium sulfate and concentrated under reduced pressure. The crude material was washed with EtOAc and dried to afford 3-(6-bromo-2-oxo-benzo[cd]indol-1-yl)piperidine-2,6-dione 3 (30 g, 59.07 mmol, 36.63% yield, 70.72% purity) as a pale yellow solid.

Step 3: To a 250 mL sealed-tube containing a well-stirred solution of 3-(6-bromo-2-oxo-benzo[cd]indol-1-yl)piperidine-2,6-dione 3 (2.00 g, 5.57 mmol) and potassium [[(tert-butoxycarbonyl)amino]methyl]trifluoroborate (3.30 g, 13.92 mmol) in 1,4-dioxane (60 mL), were added cesium carbonate (5.44 g, 16.71 mmol) and water (8 mL). The resulting solution was degassed by N₂ gas for 10 mins. Then palladium(II) acetate (125.01 mg, 556.83 μmol) and di(1-adamantyl)-n-butylphosphine (99.82 mg, 278.42 μmol) were added and again degassed by N₂ gas for 5 min and the reaction mixture was heated to 100° C. for 16 h. After completion of the reaction (TLC), the crude mixture was cooled to 0° C., quenched with saturated NH₄Cl solution slowly, and extracted with ethyl acetate (2×75 mL). The combined organic layers were dried over anhydrous Na₂SO₄, filtered, and concentrated under reduced pressure to give the crude product. The crude product was purified by flash column chromatography (Silica gel, 230-400 mesh) using 0-100% ethyl acetate in pet ether with the desired compound eluting at 50-60% ethyl acetate in pet ether to afford tert-butyl N-[[1-(2,6-dioxo-3-piperidyl)-2-oxo-benzo[cd]indol-6-yl]methyl]carbamate 4 (1.0 g, 2.21 mmol, 39.68% yield, 90.47% purity) as a yellow solid. LC-MS (ESI): m/z 354.0 [M-56+H]+.

Step 4: To a 100 mL single-necked round-bottomed flask containing a well-stirred solution of tert-butyl N-[[1-(2,6-dioxo-3-piperidyl)-2-oxo-benzo[cd]indol-6-yl]methyl]carbamate 4 (1.70 g, 4.15 mmol) in anhydrous DCM (20 mL), was added hydrogen chloride solution 4.0 M in dioxane (4 M, 5.0 mL) at 0° C. The resulting mixture were stirred at room temperature for 1 h. After consumption of starting materials, excess solvents were removed. The crude product was washed with diethyl ether (10 mL) and dried to afford 3-[6-(aminomethyl)-2-oxo-benzo[cd]indol-1-yl]piperidine-2,6-dione hydrochloride 5 (1.5 g, 4.00 mmol, 96.25% yield, 92.12% purity) as a yellow solid. Used without further purification.

Step 5: To a stirred solution of 12-tert-butoxy-12-oxo-dodecanoic acid (224.78 mg, 784.85 μmol) in DMF (10 mL) was added diisopropylethylamine (608.62 mg, 4.71 mmol, 820.24 μL) followed by HATU (447.63 mg, 1.18 mmol) and the resultant mixture was stirred for 10 min before the addition of 3-[6-(aminomethyl)-2-oxo-benzo[cd]indol-1-yl]piperidine-2,6-dione hydrochloride 5 (0.300 g, 784.85 μmol). The resulting reaction mixture stirred for 16 hr at room temperature. Water was added to the reaction mixture and extracted with ethyl acetate The organic layers were combined and concentrated then the crude material was purified by reverse phase chromatography (C18, water: ACN) to afforded tert-butyl 12-[[1-(2,6-dioxo-3-piperidyl)-2-oxo-benzo[cd]indol-6-yl]methylamino]-12-oxo-dodecanoate 6 (0.180 g, 273.53 μmol, 34.85% yield, 87.79% purity).

Step 6: Into a 100 mL single-necked round-bottomed flask containing a well-stirred solution of tert-butyl 12-[[1-(2,6-dioxo-3-piperidyl)-2-oxo-benzo[cd]indol-6-yl]methylamino]-12-oxo-dodecanoate 6 (1000.00 mg, 1.73 mmol) in anhydrous DCM (10 mL), was added hydrogen chloride, 4M in 1,4-dioxane, 99% (4 M, 5.0 mL) at 0° C. The resulting mixture was stirred at room temperature for 2 h and monitored by TLC. After consumption of starting materials, excess solvents were removed from the reaction mixture under reduced pressure to afford a crude product.

The crude product was triturated with diethyl ether (10 mL) and dried to afford 12-[[1-(2,6-dioxo-3-piperidyl)-2-oxo-benzo[cd]indol-6-yl]methylamino]-12-oxo-dodecanoic acid Compound 1 (720 mg, 1.22 mmol, 70.73% yield, 94.89% purity) as a yellow solid. LCMS (ESI): m/z 522.3 [M+H]⁺.

### Example 2. Synthesis of N₁-(2-((4-((6-(2,3-dihydrobenzo[b][1,4]dioxin-5-yl)-2-methoxypyridin-3-yl)amino)benzyl)(methyl)amino)ethyl)-N₁₂-((1-(2,6-dioxopiperidin-3-yl)-2-oxo-1,2-dihydrobenzo[cd]indol-6-yl)methyl)dodecanediamide (Compound 2)

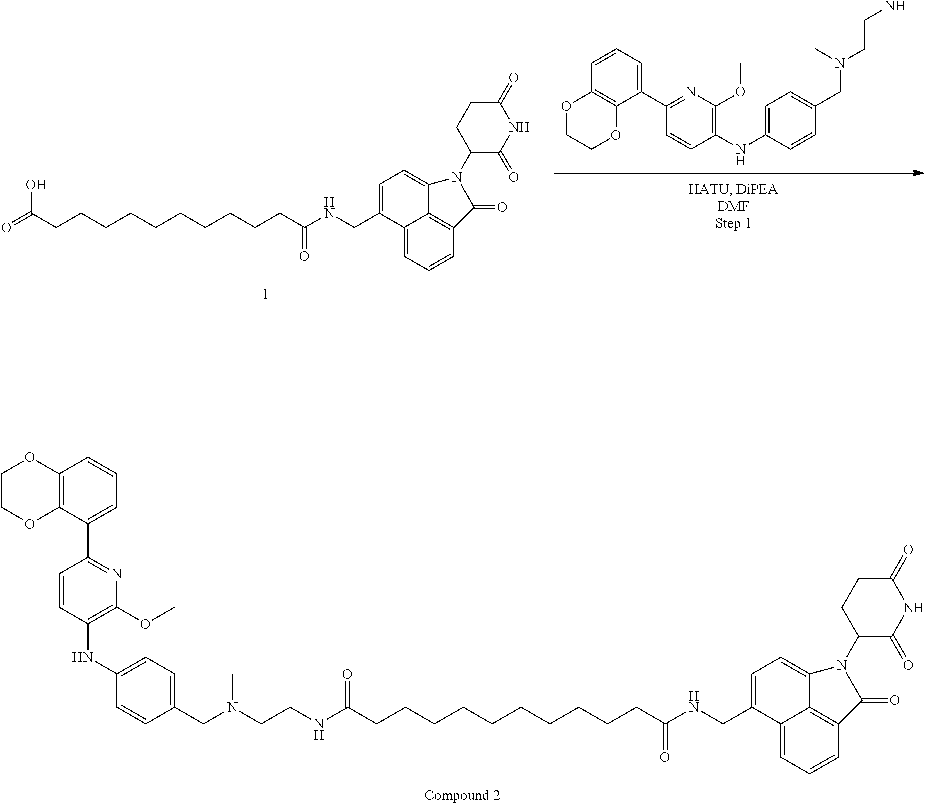 [SMILES: O=C(O)CCCCCCCCCCC(=O)NCc1ccc2c3c(cccc13)C(=O)N2C1CCC(=O)NC1=O ; COc1nc(-c2cccc3c2OCCO3)ccc1Nc1ccc(CN(C)CCN)cc1 ; COc1nc(-c2cccc3c2OCCO3)ccc1Nc1ccc(CN(C)CCNC(=O)CCCCCCCCCCC(=O)NCc2ccc3c4c(cccc24)C(=O)N3C2CCC(=O)NC2=O)cc1]

Step 1: A solution of 12-[[1-(2,6-dioxo-3-piperidyl)-2-oxo-benzo[cd]indol-6-yl]methylamino]-12-oxo-dodecanoic acid 1 (23 mg, 0.045 mmol), N₁-(4-((6-(2,3-dihydrobenzo[b][1,4]dioxin-5-yl)-2-methoxypyridin-3-yl)amino)benzyl)-N₁-methylethane-1,2-diamine (20 mg, 0.045 mmol), HATU (26 mg, 0.067 mmol) and DIPEA (23 μL, 0.13 mmol) was stirred in DMF (1 ml) at room temperature for 1 hour. The resulting reaction mixture was quenched with water (1.5 mL), extracted with ethyl acetate (3×3 mL) and evaporated to dryness. The crude material was purified by mass-based preparative HPLC [Column: X select C18 (250*19) mm, 5 microns, Mobile phase: A: 0.1% HCOOH in water, B: Acetonitrile] to afford N1-(2-((4-((6-(2,3-dihydrobenzo[b][1,4]dioxin-5-yl)-2-methoxypyridin-3-yl)amino)benzyl)(methyl)amino)ethyl)-N₁₂-((1-(2,6-dioxopiperidin-3-yl)-2-oxo-1,2-dihydrobenzo[cd]indol-6-yl)methyl)dodecanediamide formic acid salt Compound 2 (7 mg, 19%). LC-MS (ESI): m/z 902.5 [M+H]+.

### Example 3. Synthesis of N₁-((1-(2,6-dioxopiperidin-3-yl)-2-oxo-1,2-dihydrobenzo[cd]indol-6-yl)methyl)-N₁₂-(2-((4-((2-fluoro-3,3′-dimethoxy-[1,1′-biphenyl]-4-yl)amino)benzyl)(methyl)amino)ethyl)dodecanediamide (Compound 3)

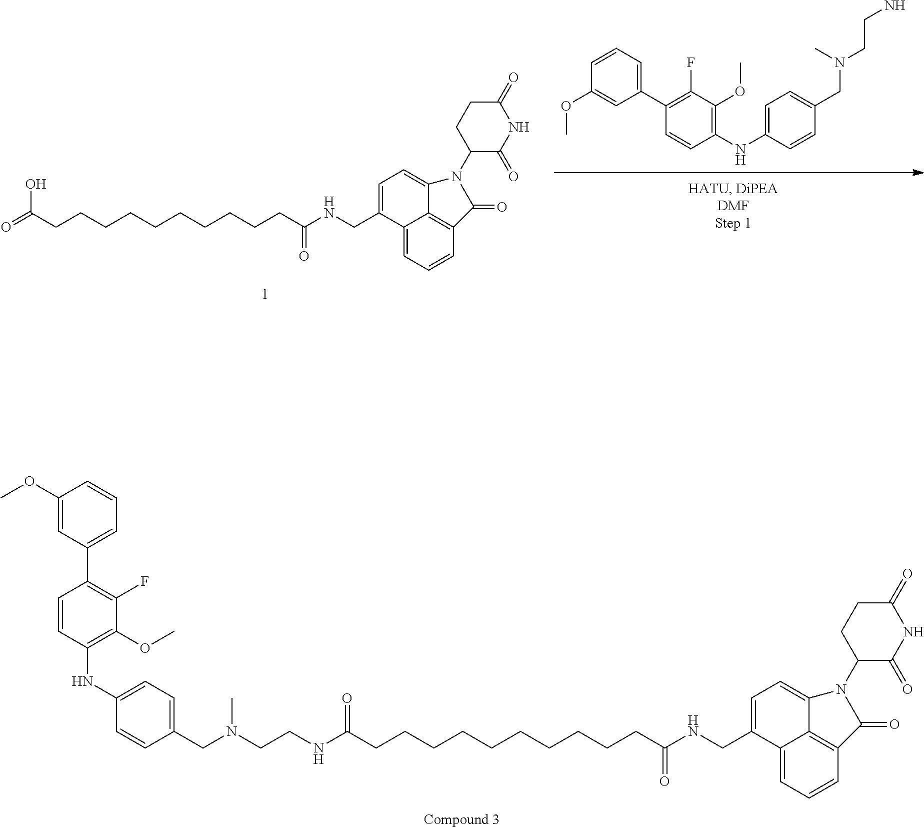 [SMILES: O=C(O)CCCCCCCCCCC(=O)NCc1ccc2c3c(cccc13)C(=O)N2C1CCC(=O)NC1=O ; COc1cccc(-c2ccc(Nc3ccc(CN(C)CCN)cc3)c(OC)c2F)c1 ; COc1cccc(-c2ccc(Nc3ccc(CN(C)CCNC(=O)CCCCCCCCCCC(=O)NCc4ccc5c6c(cccc46)C(=O)N5C4CCC(=O)NC4=O)cc3)c(OC)c2F)c1]

Step 1: A solution of 12-[[1-(2,6-dioxo-3-piperidyl)-2-oxo-benzo[cd]indol-6-yl]methylamino]-12-oxo-dodecanoic acid 1 (23 mg, 0.045 mmol), N₁-(4-((6-(2,3-dihydrobenzo[b][1,4]dioxin-5-yl)-2-methoxypyridin-3-yl)amino)benzyl)-N₁-methylethane-1,2-diamine (20 mg, 0.045 mmol), HATU (26 mg, 0.067 mmol) and DIPEA (23 μL, 0.13 mmol) was stirred in DMF (1 ml) at room temperature for 1 hour. The resulting reaction mixture was quenched with water (1.5 mL), extracted with ethyl acetate (3×3 mL) and evaporated to dryness. The crude material was purified by mass-based preparative HPLC [Column: X select C18 (250*19) mm, 5 microns, Mobile phase: A: 0.1% HCOOH in water, B: Acetonitrile] to give N₁-((1-(2,6-dioxopiperidin-3-yl)-2-oxo-1,2-dihydrobenzo[cd]indol-6-yl)methyl)-N12-(2-((4-((2-fluoro-3,3′-dimethoxy-[1,1′-biphenyl]-4-yl)amino)benzyl)(methyl)amino)ethyl)dodecanediamide Compound 3 (9 mg, 21%). LC-MS (ESI): m/z 914.4 [M+H]+.

### Example 4. Synthesis of afford N′-[[4-[[6-(2,3-dihydro-1,4-benzodioxin-5-yl)-2-methoxy-3-pyridyl]amino]phenyl]methyl]-N′-methyl-ethane-1,2-diamine trifluoroacetate (Compound 4)

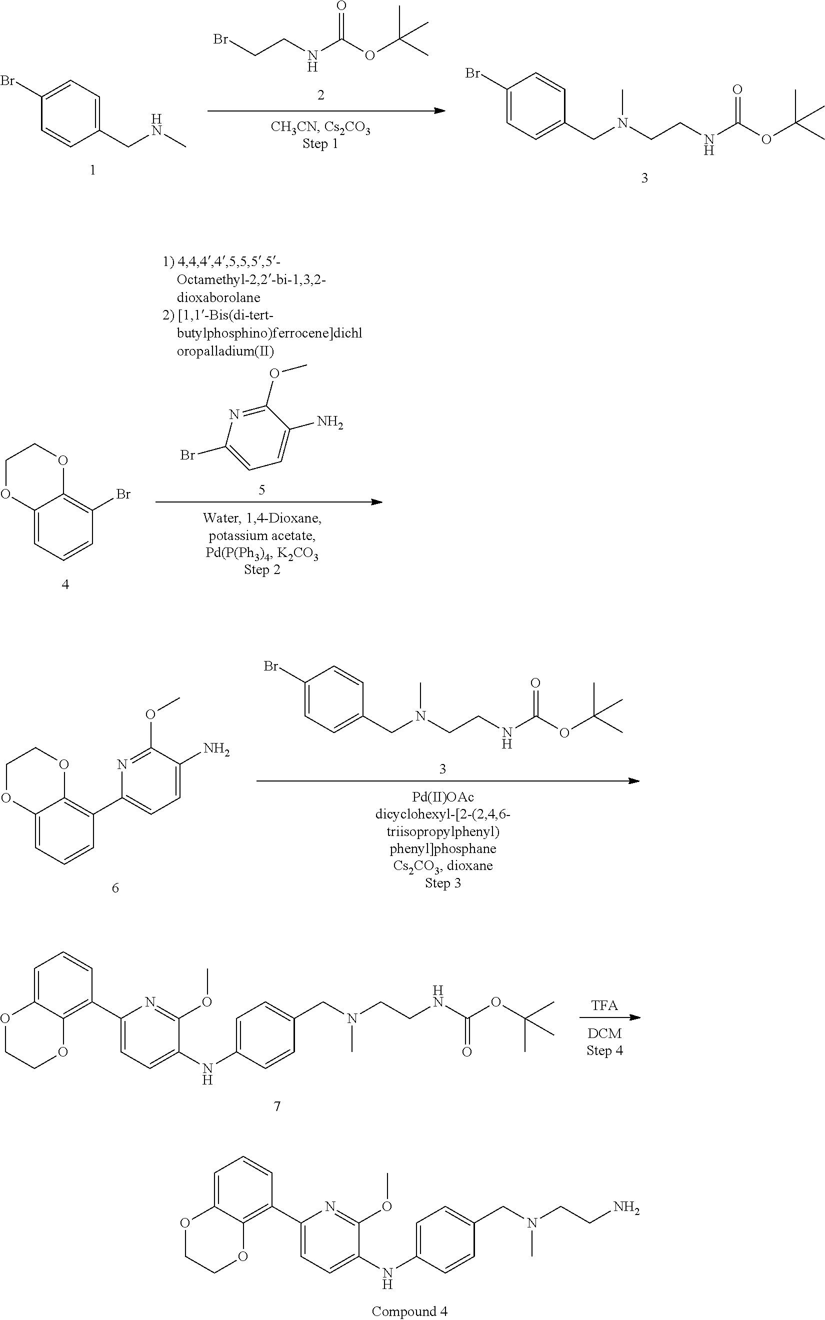

Step 1: To a 250 mL sealed tube containing a well-stirred solution of a mixture of 1-(4-bromophenyl)-N-methyl-methanamine 1 (5 g, 24.99 mmol, 5.00 mL) in anhydrous ACN (10 mL) was added cesium carbonate (16.28 g, 49.98 mmol) at room temperature. The resulting reaction mixture was stirred at 60° C. for 24 h. The progress of the reaction was monitored by TLC. After completion of the reaction, the reaction mixture was filtered through celite and washed with EtOAc (1000 mL). The solution was dried under reduced pressure to afford the crude product tert-butyl N-[2-[(4-bromophenyl)methyl-methyl-amino]ethyl]carbamate 3 (8.5 g, 24.76 mmol, 99.09% yield) as a pale yellow oil.

Step 2: Into a 100 mL sealed tube containing a well-stirred solution of 5-bromo-2,3-dihydro-1,4-benzodioxine 4 (1000.00 mg, 4.65 mmol) and 4,4,4′,4′,5,5,5′,5′-octamethyl-2,2′-bi-1,3,2-dioxaborolane (1.77 g, 6.98 mmol) in 1,4-dioxane (20 mL), was added potassium acetate (1.14 g, 11.63 mmol, 726.72 μL) then degassed by N₂ gas for 10 mins. Then [1,1′-bis(di-tert-butylphosphino)ferrocene]dichloropalladium(II) (303.07 mg, 465.02 μmol) was added, the mixture again degassed by N₂ gas for 5 min, then heated to 120° C. for 3h. To the reaction mixture was then added 6-bromo-2-methoxy-pyridin-3-amine 5 (944.16 mg, 4.65 mmol) potassium carbonate (1.29 g, 9.30 mmol, 561.30 μL), water (4 mL) and Pd(PPh₃)₄ (537.36 mg, 465.02 μmol). This solution was degassed by N₂ gas for 10 mins and heated to 110° C. for 3 h. After completion of the reaction (TLC), the crude mixture was cooled to 0° C., quenched with water (10 mL) and extracted with ethyl acetate (2×75 mL). The combined organic layers were dried over anhydrous Na₂SO₄, filtered, and concentrated under reduced pressure to give the crude product. The crude product was purified by flash column chromatography (silica gel, 230-400 mesh) using 0-60% ethyl acetate in pet ether with the desired compound eluting at 30-40% ethyl acetate in pet ether to afford 6-(2,3-dihydro-1,4-benzodioxin-5-yl)-2-methoxy-pyridin-3-amine 6 (600 mg, 1.90 mmol, 40.84% yield, 81.75% purity) as a light brown solid. LC-MS (ESI): m/z 259.0 [M+H]+.

Step 3: Into a 250 mL sealed tube containing a well-stirred solution of 6-(2,3-dihydro-1,4-benzodioxin-5-yl)-2-methoxy-pyridin-3-amine 6 (1.50 g, 5.83 mmol) and tert-butyl N-[2-[(4-bromophenyl)methyl-methyl-amino]ethyl]carbamate 3 (2.00 g, 5.83 mmol) in 1,4-dioxane (30 mL), was added cesium carbonate (5.70 g, 17.48 mmol). This mixture was degassed by N₂ gas for 10 mins, then palladium(II) acetate (13.08 mg, 58.27 μmol) and dicyclohexyl-[2-(2,4,6-triisopropylphenyl)phenyl]phosphane (83.33 mg, 174.80 μmol) were added and again degassed by N₂ gas for 5 min. The reaction mixture was heated to 110° C. for 16 h. After consumption of starting materials (TLC), the reaction mix was diluted with ethyl acetate (100 mL) and water (20 mL), the organic layers separated, dried over anhydrous Na₂SO₄, filtered and concentrated under reduced pressure to give the crude product. The crude product was purified by flash column chromatography (silica gel, 230-400 mesh) using 0-100% ethyl acetate in pet ether while the desired compound eluting at 90-100% ethyl acetate in pet ether to afford tert-butyl N-[2-[[4-[[6-(2,3-dihydro-1,4-benzodioxin-5-yl)-2-methoxy-3-pyridyl]amino]phenyl]methyl-methyl-amino]ethyl]carbamate 7 (1.2 g, 2.04 mmol, 34.93% yield, 88.30% purity) as a thick brown liquid. LC-MS (ESI): m/z 521.3[M+H]+.

Step 4: Into a 100 mL single-necked round-bottom flask containing a well-stirred solution of tert-butyl N-[2-[[4-[[6-(2,3-dihydro-1,4-benzodioxin-5-yl)-2-methoxy-3-pyridyl]amino]phenyl]methyl-methyl-amino]ethyl]carbamate 7 (700 mg, 1.34 mmol) in anhydrous DCM (5 mL) was added TFA (1.53 g, 13.45 mmol, 1.04 mL) at 0° C. The resulting mixture was stirred at room temperature for 1 h. After consumption of starting materials (TLC), excess solvents were removed from the reaction mixture under reduced pressure to give a crude product. The crude product was triturated with diethyl ether (10 mL) and dried to afford N′-[[4-[[6-(2,3-dihydro-1,4-benzodioxin-5-yl)-2-methoxy-3-pyridyl]amino]phenyl]methyl]-N′-methyl-ethane-1,2-diamine trifluoroacetate Compound 4 (700 mg, 1.18 mmol, 87.66% yield, 90% purity) as a brown liquid. LC-MS (ESI): m/z 421.3 [M+H]+.

### Example 5. Synthesis of N′-[[4-[3-fluoro-2-methoxy-4-(3-methoxyphenyl)anilino]phenyl]methyl]-N′-methyl-ethane-1,2-diamine (Compound 5)

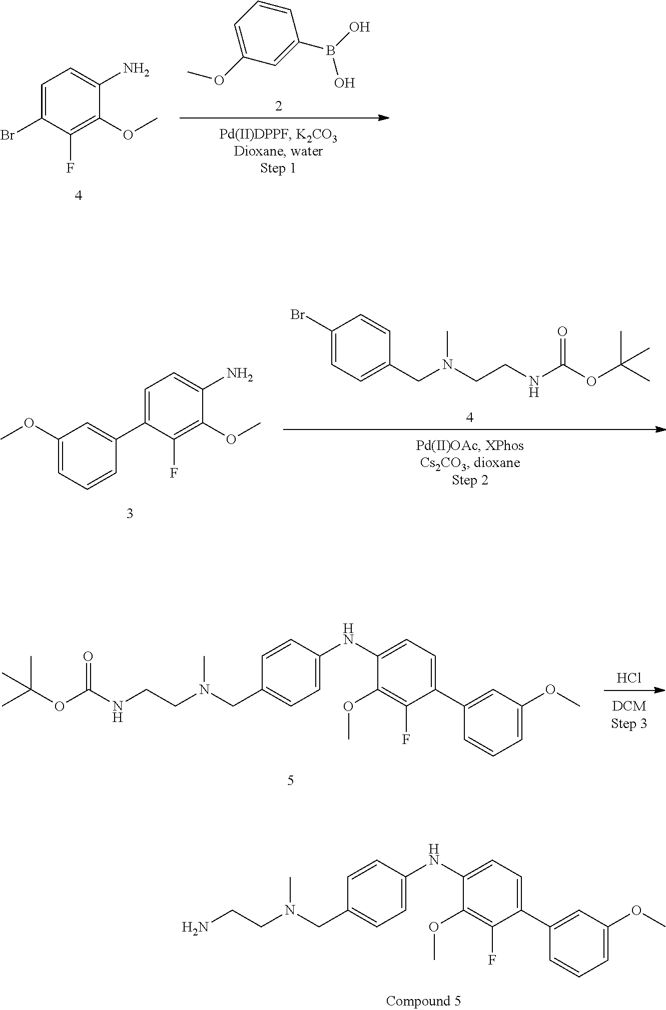

Step 1: To a 100 mL sealed tube containing a well-stirred solution of 4-bromo-3-fluoro-2-methoxy-aniline 1 (2 g, 9.09 mmol), (3-methoxyphenyl)boronic acid 2 (1.52 g, 10.00 mmol) in anhydrous 1,4-dioxane (15 mL) was added potassium carbonate (3.77 g, 27.27 mmol, 1.65 mL) at ambient temperature. The resulting solution was degassed for 10 min with N₂ gas. To that solution was added Pd(dppf)Cl₂·CH₂Cl₂ (371.13 mg, 454.47 μmol) and the reaction mixture was stirred at 100° C. for 6 h. After completion of the reaction, the reaction mixture was cooled to room temperature. The reaction mixture was filtered through a celite pad and washed with EtOAc (300 mL). The filtrate was washed with water (2×100 mL), then the organic phase was separated, washed with brine solution (200 mL), and dried over anhydrous Na₂SO₄. The solution was filtered and dried under reduced pressure to give the crude product which was purified by flash column chromatography (silica gel, 230-400 mesh) eluting with 0-50% EtOAc:pet ether and the compound eluted in 8-10% EtOAc:Pet ether to afford 3-fluoro-2-methoxy-4-(3-methoxyphenyl)aniline 3 (1.3 g, 5.08 mmol, 55.88% yield, 96.60% purity) as a pale yellow oil. LCMS (ESI): m/z 248.0 [M+H]+.

Step 2: To a 50 mL sealed tube reactor containing a well-stirred solution of 3-fluoro-2-methoxy-4-(3-methoxyphenyl)aniline 3 (1.04 g, 4.20 mmol) and tert-butyl N-[2-[(4-bromophenyl)methyl-methyl-amino]ethyl]carbamate 4 (1.2 g, 3.50 mmol) in anhydrous 1,4-dioxane (12 mL) was added cesium carbonate (3.42 g, 10.49 mmol) at room temperature under nitrogen atmosphere. The resulting mixture was degassed by bubbling nitrogen gas into the reaction mixture for 10 minutes. Subsequently, XPhos (166.65 mg, 349.59 μmol), followed by palladium(II) acetate (39.24 mg, 174.80 μmol), was added. The resulting reaction mixture was heated to 100° C. for 6 h. The reaction mixture was filtered through celite and washed with EtOAc (250 mL). The filtrate was washed with water (2×100 mL), then the organic phase was separated, washed with brine solution (100 mL) and dried over anhydrous Na₂SO₄. The solution was filtered and dried under reduced pressure to give the crude product which was purified by column chromatography (silica gel, 230-400 mesh) eluting with 0-10% MeOH:DCM. The compound eluted in 2-5% MeOH:DCM to afford tert-butyl N-[2-[[4-[3-fluoro-2-methoxy-4-(3-methoxyphenyl)anilino]phenyl]methyl-methyl-amino]ethyl]carbamate 5 (1.6 g, 2.80 mmol, 80.05% yield, 89.13% purity) as a reddish brown gummy solid. LCMS (ESI): m/z 510.3 [M+H]⁺. Used without further purification.

Step 3: To a 100 mL single-necked round bottom flask containing a well-stirred solution of tert-butyl N-[2-[[4-[3-fluoro-2-methoxy-4-(3-methoxyphenyl)anilino]phenyl]methyl-methyl-amino]ethyl]carbamate 5 (700 mg, 1.37 mmol) in anhydrous DCM (5 mL), was added a hydrogen chloride solution 4.0M in dioxane (4 M, 2.04 mL) at 0° C. The resulting mixture was stirred at room temperature for 1 h. Solvents were removed from the reaction mixture under reduced pressure to give a crude product. The crude product was triturated with diethyl ether (10 mL) and dried to afford N′-[[4-[3-fluoro-2-methoxy-4-(3-methoxyphenyl)anilino]phenyl]methyl]-N′-methyl-ethane-1,2-diamine Compound 5 (610 mg, 1.33 mmol, 96.56% yield, 96.97% purity) as a yellow solid. LC-MS (ESI): m/z 410.3 [M+H]+.

### Example 6. Synthesis of 3-[5-(aminomethyl)-2-oxo-benzo[cd]indol-1-yl]piperidine-2,6-dione hydrochloride (Compound 6)

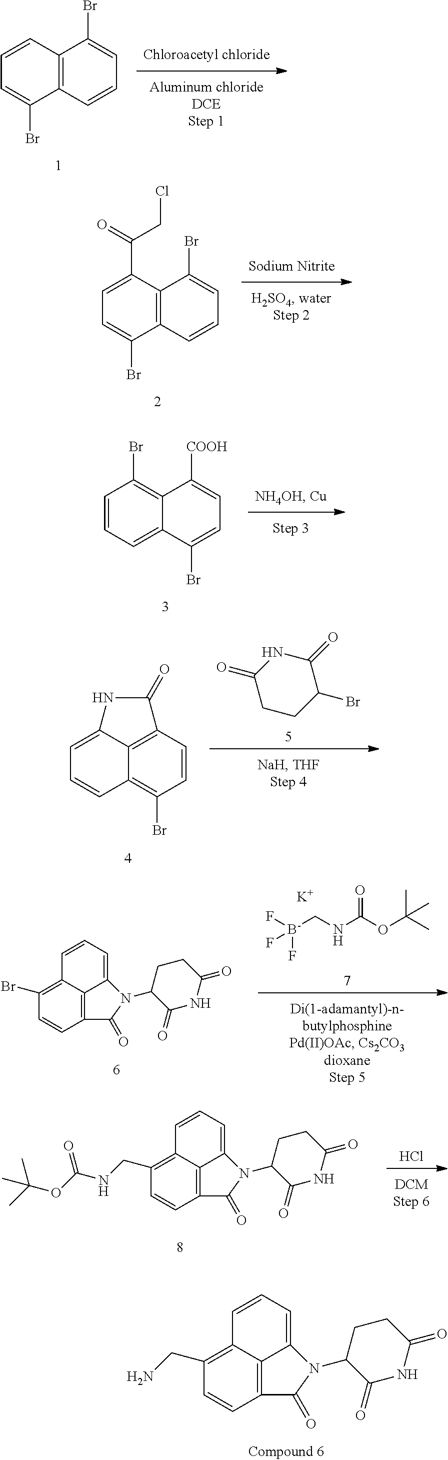

Step 1: A stirred solution of 1,5-dibromonaphthalene 1 (120 g, 419.64 mmol, 000) in DCE (1440 mL) was cooled to 0° C. and chloroacetyl chloride (61.61 g, 545.53 mmol, 43.39 mL) added dropwise. The reaction mixture was stirred at this temperature for about 15 minutes. Aluminum trichloride (72.74 g, 545.53 mmol, 29.81 mL) was then added portionwise and the reaction mixture was slowly warmed to RT and stirred for 5 hr. The reaction mixture was quenched with cold water (500 mL) and DCM (1200 mL) then filtered through celite. The filtrate was washed with water and brine, and the DCM layer was dried over anhydrous sodium sulfate, filtered, and concentrated under reduced pressure to obtain the crude solid. This crude material was stirred in 2% ethyl acetate in pet ether (1200 mL) for 30 min and the solid filtered and washed with pet ether (1200 mL) to afford 2-chloro-1-(4,8-dibromo-1-naphthyl)ethenone 2 (110 g, 294.39 mmol, 70.15% yield, 97% purity) as a light green solid. TLC: Rf: 0.3, 10% EtOAc in Pet ether, UV detection.

Step 2: To a stirred solution of 2-chloro-1-(4,8-dibromo-1-naphthyl)ethenone 2 (200 g, 551.81 mmol) in H₂SO₄ (2400 mL) was added a solution of sodium nitrite (39.98 g, 579.40 mmol, 18.42 mL) in water (40 mL) dropwise at 0° C. and the resultant reaction mixture was stirred at 25° C. for 2h. The reaction mixture was then poured into cold water (870 mL) and filtered. The solid thus obtained was added to an ethyl acetate and water solution (1:1, 870:870 mL), the mixture was filtered over celite and washed with ethyl acetate (500 mL). The aqueous layer was extracted with ethyl acetate (2×100 mL). The combined organic layers were then washed with brine, dried over anhydrous sodium sulfate and concentrated under reduced pressure. The crude material was washed with 10% ethyl acetate in pet ether and dried to afford 4,8-dibromonaphthalene-1-carboxylic acid 3 (160 g, 402.46 mmol, 72.93% yield, 83% purity) as a brown solid. TLC: Rf: 0.2, 50% EtOAc in Pet ether, UV detection.

Step 3: To a stirred suspension of 4,8-dibromonaphthalene-1-carboxylic acid 3 (160 g, 484.89 mmol, 000) in ammonium hydroxide (28% solution) (1.98 kg, 56.49 mol, 2.2 L), copper (8.01 g, 126.07 mmol) was added and the reaction mixture was stirred at 80° C. for 2 hr. The reaction mixture was cooled to RT and acidified with concentrated hydrochloric acid to pH 2-3. The resulting suspension was filtered and dried to afford the crude product. This crude stirred in 10% ethyl acetate in pet ether for 30 min, filtered and washed with pet ether to afford 5-bromo-1H-benzo[cd]indol-2-one 4 (105 g, 342.84 mmol, 70.70% yield, 81% purity) as a brown solid. TLC: Rf: 0.3, 70% EtOAc in Pet ether, UV detection.

Step 4: To a 500 mL three-necked round bottom flask containing a well stirred solution of 5-bromo-1H-benzo[cd]indol-2-one 4 (2.0 g, 6.85 mmol) and 5-bromo-1H-benzo[cd]indol-2-one (2.0 g, 6.85 mmol) in dry THE (200 mL) was added sodium hydride (60% dispersion in mineral oil, 2.63 g, 68.53 mmol) at 0° C. and the reaction mixture was stirred at ambient temperature. After 1 hr, 3-bromopiperidine-2,6-dione 5 (6.58 g, 30.84 mmol, 000) dissolved in dry THE (10 mL) was added at 0° C. The reaction mixture was stirred at 65° C. for 16 h. The reaction mixture was quenched with saturated aqueous ammonium chloride solution (50 mL) then extracted with ethyl acetate (2×50 mL). Organic layers were collected, dried over anhydrous sodium sulfate, and concentrated under reduced pressure. The resulting residue was then triturated with DCM (10 mL) to give 3-(5-bromo-2-oxo-benzo[cd]indol-1-yl)piperidine-2,6-dione 6 (1.5 g, 3.30 mmol, 48.14% yield, 79% purity) as a yellow solid. LCMS (ES+): m/z 359.0 [M+H]+. ¹H NMR (d6-DMSO, 400 MHz) δ 11.14 (s, 1H), 8.12 (d, J=7.48 Hz, 1H), 7.99 (d, J=7.44 Hz, 1H), 7.72-7.62 (m, 2H), 7.26 (d, J=6.92 Hz, 1H), 5.46 (dd, J=12.84, 5.28 Hz, 1H), 2.99-2.90 (m, 1H), 2.81-2.63 (m, 2H), 2.12-2.07 (m, 1H); LC MS: ES+ 359.07, 361.02 (Bromo pattern).

Step 5: To an oven dried 250 mL sealed tube was charged with 3-(5-bromo-2-oxo-benzo[cd]indol-1-yl)piperidine-2,6-dione 6 (1 g, 2.78 mmol) and potassium [[(tert-butoxycarbonyl)amino]methyl]trifluoroborate 7 (1.65 g, 6.96 mmol) in 1,4-dioxane (30 mL) and water (8 mL), was added cesium carbonate (2.72 g, 8.35 mmol). The contents were degassed with nitrogen gas for 10 minutes followed by addition of di(1-adamantyl)-n-butylphosphine (49.91 mg, 139.21 μmol) and palladium(II) acetate (62.51 mg, 278.42 μmol). The resulting mixture was stirred at 100° C. for 16 h. After completion of the reaction, the reaction mixture was diluted with water (10 mL) and extracted with ethyl acetate (3×100 mL). The combined organic layers were washed with a brine solution (20 mL), dried over anhydrous Na₂SO₄, filtered, and concentrated under reduced pressure. The crude product was purified by flash column chromatography (silica gel, 230-400 mesh) eluting with 50-60% ethyl acetate-pet ether to afford tert-butyl N-[[1-(2,6-dioxo-3-piperidyl)-2-oxo-benzo[cd]indol-5-yl]methyl]carbamate 8 (200 mg, 458.73 μmol, 16.48% yield, 93.91% purity) as a pale yellow solid. LCMS (ESI): m/z 354.0 [M+H-tBu]+.

Step 6: An oven-dried 50 mL single-necked round-bottomed flask was charged with tert-butyl N-[[1-(2,6-dioxo-3-piperidyl)-2-oxo-benzo[cd]indol-5-yl]methyl]carbamate 8 (600 mg, 1.47 mmol) and DCM (10 mL) then cooled to 0° C. A hydrogen chloride solution 4.0M in dioxane (4.80 g, 131.65 mmol, 6 mL) was then added and the resulting mixture was stirred at room temperature for 1 h. The reaction mixture was next concentrated in vacuo. The obtained crude product was washed with diethyl ether (20 mL) to afford 3-[5-(aminomethyl)-2-oxo-benzo[cd]indol-1-yl]piperidine-2,6-dione hydrochloride Compound 6 (505 mg, 1.40 mmol, 95.46% yield, 95.78% purity) as a pale yellow solid. LCMS (ESI): m/z 310.2 [M+H]+

### Example 7. Synthesis of 3-[4-(aminomethyl)-2-oxo-benzo[cd]indol-1-yl]piperidine-2,6-dione hydrochloride (Compound 7) and 3-(4-bromo-2-oxo-benzo[cd]indol-1-yl)piperidine-2,6-dione (Compound 8)

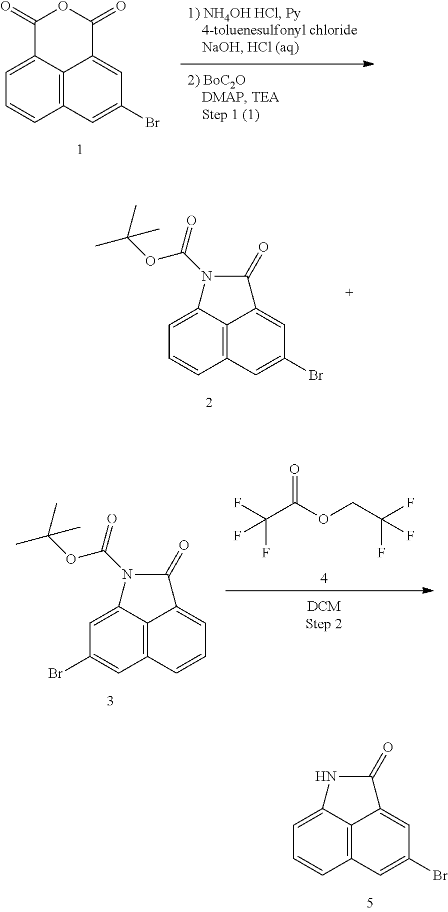 [SMILES: O=C1OC(=O)c2cc(Br)cc3cccc1c23 ; CC(C)(C)OC(=O)N1C(=O)c2cc(Br)cc3cccc1c23 ; CC(C)(C)OC(=O)N1C(=O)c2cccc3cc(Br)cc1c23 ; O=C(OCC(F)(F)F)C(F)(F)F ; "4" ; O=C1Nc2cccc3cc(Br)cc1c23]

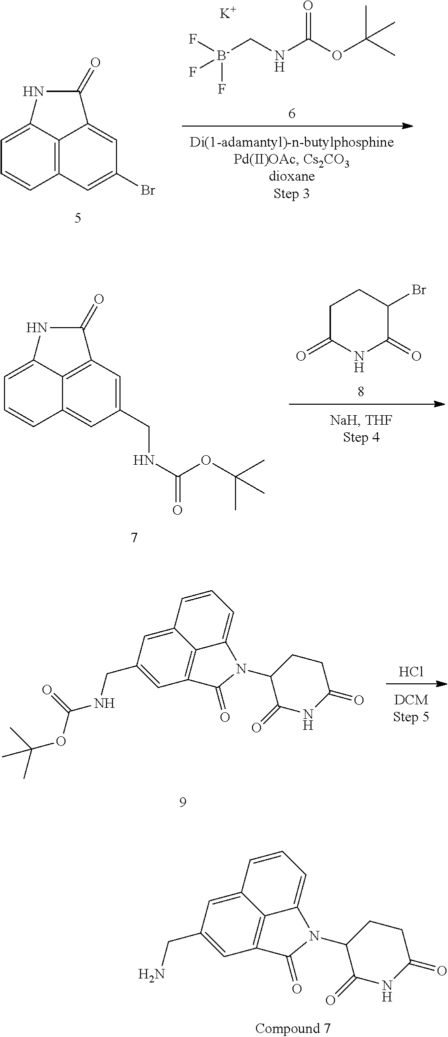

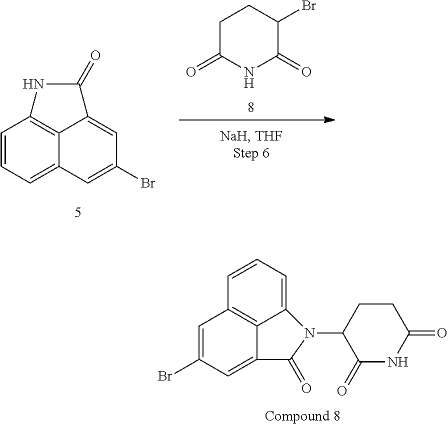

Step 1 part (1): A solution of 7-bromo-14-oxatricyclotrideca-,2(6),3(7),4(8),5(9)-pentaene-10,11-dione 1 (CAS #24050-49-5, 5 g, 18.05 mmol) and hydroxylamine hydrochloride (1.25 g, 18.05 mmol, 750.92 μL) in pyridine (36 mL) was stirred under reflux for 5 h, followed by cooling to 80° C. Then 4-toluenesulfonyl chloride (6.88 g, 36.09 mmol) was added to the reaction system. After addition, the temperature was raised and the reaction was stirred at reflux for 5 h, followed by cooling to room temperature. The reaction mixture was poured into 90 mL of water and stirred to precipitate crystals, which were collected by filtration. The crystals were transferred to a beaker and washed successively with 90 mL of a NaHCO₃aqueous solution and 90 mL of water, followed by filtration. The crystals were washed with water and dried to give an intermediate for further reaction. The whole amount of the intermediate was dissolved in EtOH (15 mL) and water (18 mL) and put in a reactor and stirred. Then sodium hydroxide (flake, 98%, 1.4 M, 60 mL) was added dropwise to the mixture. Thereafter, the mixture was heated to reflux, at which temperature the reaction was carried out for 3 hr while distilling off ethanol. After completion of the reaction, the reaction mixture was cooled to 75° C., and hydrochloric acid (36% w/w aq. soln., 8.00 g, 219.41 mmol, 10 mL) was added dropwise. Crystals precipitated at 60° C. After completion of the dropwise addition, the mixture was further cooled. The precipitated crystals were collected by filtration, washed with water, and dried to afford a regioisomeric mixture of 4-bromo-1H-benzo[cd]indol-2-one and 7-bromo-1H-benzo[cd]indol-2-one as a yellow solid. Used in the next step without further purification.

Step 1 part (2): To a stirred solution of 4-bromo-1H-benzo[cd]indol-2-one and 7-bromo-1H-benzo[cd]indol-2-one (3 g, 12.1 mmol) (regio-isomer mixture) in DCM (30 mL) was added triethylamine (1.84 g, 18.14 mmol, 2.53 mL) and DMAP (73.87 mg, 604.66 μmol) at RT, followed by the addition of tert-butoxycarbonyl tert-butyl carbonate (1.98 g, 9.07 mmol, 2.08 mL) at 0° C. The cooling bath was then removed and the reaction mixture stirred at RT for 3h. The reaction mixture was poured into water and extracted with DCM, dried over Na₂SO₄, filtered, and solvent removed under reduced pressure. The crude compound was purified by column chromatography (silica gel; 4% ethyl acetate-pet ether) to give tert-butyl 4-bromo-2-oxo-benzo[cd]indole-1-carboxylate 2 (1 g, 2.77 mmol, 45.87% yield, 96.58% purity) as an off white solid and tert-butyl 7-bromo-2-oxo-benzo[cd]indole-1-carboxylate 3 (1.1 g, 1.88 mmol, 31.07% yield, 59.47% purity) as an off white solid.

Step 2: To the stirred solution of tert-butyl 4-bromo-2-oxo-benzo[cd]indole-1-carboxylate 2 (2.0 g, 5.74 mmol) in DCM (15 mL) was added (2,2,2-trifluoroethyl) 2,2,2-trifluoroacetate 4 (12.06 g, 57.44 mmol, 8.10 mL) over a period of 5 minutes at 0° C. The reaction mixture was warmed to room temperature and stirred at this temperature for 3 h. The reaction mixture was concentrated under reduced pressure at 45° C. The crude product was triturated using diethyl ether to afford desired product of 4-bromo-1H-benzo[cd]indol-2-one 5 (1.9 g, 7.66 mmol, 133.34% yield) as greenish liquid. The crude product was taken for next step without any further purification.

Step 3: To a 250 mL sealed tube containing a well-stirred suspension of 4-bromo-1H-benzo[cd]indol-2-one 5 (1.5 g, 6.05 mmol), potassium ((tert-butoxycarbonyl)amino)methyl-trifluoroborate 6 (3.58 g, 15.12 mmol) in 1,4-dioxane (45 mL) and water (15 mL) was added cesium carbonate (5.91 g, 18.14 mmol), di(1-adamantyl)-n-butylphosphine (108.40 mg, 302.33 μmol) and palladium(II) acetate (135.75 mg, 604.66 μmol) at ambient temperature under nitrogen. The resulting mixture was stirred at 100° C. for 16 h. The reaction mixture was cooled to ambient temperature, quenched with water (5 mL), extracted with ethyl acetate (3×60 mL), and the combined organic layers washed with brine (30 mL), dried over anhydrous sodium sulfate, filtered and concentrated under reduced pressure to give a crude residue. The crude compound was purified by flash column chromatography (silica gel, 230-400 mesh) eluting with 50-60% ethyl acetate in petroleum ether to give tert-butyl N-[(2-oxo-1H-benzo[cd]indol-4-yl)methyl]carbamate 7 (1.3 g, 4.05 mmol, 67.02% yield, 93% purity) as a pale yellow solid. LC-MS (ESI) m/z: 243.2 [M-tBu+H]+.

Step 4: To a 500 mL three-necked round bottom flask containing a well-stirred suspension of tert-butyl N-[(2-oxo-1H-benzo[cd]indol-4-yl)methyl]carbamate 7 (2.6 g, 8.72 mmol) in tetrahydrofuran (150 mL) was added sodium hydride (60% dispersion in mineral oil, 2.58 g, 64.49 mmol) at 0° C. under nitrogen. The reaction mixture was allowed to stir at ambient temperature for 1 hr. To the reaction mixture was then added 3-bromopiperidine-2,6-dione 8 (5.35 g, 27.89 mmol) in tetrahydrofuran (15 mL) at 0° C. The reaction mixture was stirred at 65° C. for 4 h. The reaction mixture was cooled to 0° C., quenched with saturated ammonium chloride solution (30 mL), extracted with ethyl acetate (3×150 mL), and the combined organic layers dried over anhydrous sodium sulfate, filtered and concentrated under reduced pressure to get crude residue. The crude compound was purified by column chromatography (Silica gel, 230-400 mesh) eluting with 40-60% ethyl acetate in petroleum ether to get tert-butyl N-[[1-(2,6-dioxo-3-piperidyl)-2-oxo-benzo[cd]indol-4-yl]methyl]carbamate 9 (2.6 g, 5.91 mmol, 67.76% yield, 93% purity) as a yellow solid. LC-MS (ESI) m/z: 408.0 [M−H]−.

Step 5: To a 100 mL round bottom flask containing a well stirred solution of tert-butyl N-[[1-(2,6-dioxo-3-piperidyl)-2-oxo-benzo[cd]indol-4-yl]methyl]carbamate 9 (1 g, 2.44 mmol) in DCM (10 mL) was added 4M HCl in 1,4-dioxane (89.05 mg, 2.44 mmol, 10 mL) dropwise at 0° C. After addition the cooling bath was removed and the reaction mixture stirred at room temperature for 2 hr. The reaction mixture was concentrated under reduced pressure to give crude material which was triturated with diethyl ether (10 mL) and dried to afford 3-[4-(aminomethyl)-2-oxo-benzo[cd]indol-1-yl]piperidine-2,6-dione hydrochloride Compound 7 (800 mg, 2.17 mmol, 89.04% yield, 94% purity) LCMS (ESI): m/z 310.2 [M+H]+.

Step 6: To a stirred solution of 4-bromo-1H-benzo[cd]indol-2-one 2 (5 g, 20.16 mmol) in THE (50 mL) was added sodium hydride (4.84 g, 201.55 mmol) at 0° C. under nitrogen atmosphere. Reaction mixture was stirred for 2 hr at room temperature. The reaction mixture was cooled to 0° C. and 3-bromopiperidine-2,6-dione 8 (19.35 g, 100.78 mmol) was added in portions at 0° C. under nitrogen atmosphere. The reaction mixture was then heated to 65° C. and stirred at this temperature for 2 hr at 65° C. Water (100 mL) and EtOAc (50 mL) were added and the layers separated, and the aqueous layer was extracted with EtOAc (50 mL). The combined organic layers were washed with brine (25 mL), dried over anhydrous sodium sulfate and concentrated under reduced pressure to afford 3-(4-bromo-2-oxo-benzo[cd]indol-1-yl)piperidine-2,6-dione Compound 8 (3.0 g, 7.59 mmol, 37.66% yield, 90.88% purity). ¹H NMR (d6-DMSO, 400 MHZ) δ 11.14 (s, 1H), 8.52 (s, 1H), 8.23 (s, 1H), 7.65-7.55 (m, 2H), 7.22-7.19 (m, 1H), 5.47-5.44 (m, 1H), 2.96-2.90 (m, 1H), 2.77-2.63 (m, 2H), 2.13-2.11 (m, 1H); LC MS: ES+ 358.9, 361.1.

### Example 8. Synthesis of 3-(6-bromo-2-oxopyrrolo[4,3,2-ij]isoquinolin-1(2H)-yl)piperidine-2,6-dione (Compound 9)

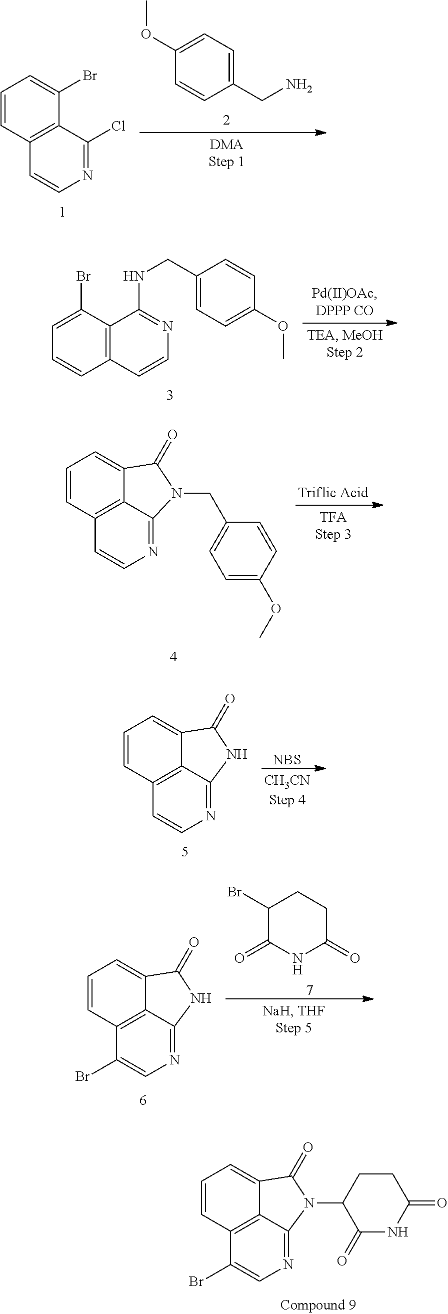

Step 1: A solution of 8-bromo-1-chloro-isoquinoline 1 (50 g, 206.19 mmol) and 4-methoxy benzylamine 2 (42.43 g, 309.28 mmol, 40.41 mL) in DMA (300 mL) in a sealed vessel was heated at 120° C. for 3 hr. The reaction mixture was diluted with ethyl acetate and water. The organic layer was dried over anhydrous sodium sulfate and concentrated. The reaction mixture was purified by silica gel column chromatography (5% ethyl acetate in hexane) to afford 8-bromo-N-(4-methoxybenzyl)isoquinolin-1-amine 3 (52g, 72%).

Step 2: To a solution of 8-bromo-N-[(4-methoxyphenyl)methyl]isoquinolin-1-amine 3 (52 g, 151.51 mmol) in MeOH (500 mL) was added triethylamine (61.32 g, 606.03 mmol, 84.47 mL) then the vessel purged with argon for 10 minutes. DPPP (12.50 g, 30.30 mmol) and palladium(II) acetate (3.40 g, 15.15 mmol) was added and the reaction mixture was shaken in a Parr-autoclave at 100° C. under an atmosphere of 70 psi of carbon monoxide. The reaction mixture was filtered through a celite bed and concentrated. The crude material was diluted with ethyl acetate and washed with water followed by brine. The organic layer was dried over anhydrous sodium sulfate and concentrated. the crude material was purified by silica gel column chromatography (60% ethyl acetate in hexane) to afford the 19-[(4-methoxyphenyl)methyl]-18,19-diazatricyclododeca-1(3),2(12),8,14,16(18)-pentaen-17-one 4 (44 g, 90%) as an off white solid.

Step 3: To a cooled solution of 19-[(4-methoxyphenyl)methyl]-18,19-diazatricyclododeca-1(3),2(12),8,14,16(18)-pentaen-17-one 4 (1 g, 3.44 mmol) in TFA (12 mL) was added triflic acid (3.62 g, 24.11 mmol, 2.12 mL) dropwise. The cooling bath was removed and the reaction mixture was stirred at 25° C. for 14 hr. The reaction mixture was evaporated and quenched with saturated sodium bicarbonate solution, extracted with ethyl acetate, and the combined organic layers were washed with water followed by brine. The organic portion was dried over anhydrous sodium sulfate and concentrated to give 10,11-diazatricyclododeca-(2),1(5),3,6,8(10)-pentaen-9-one 5 (580 mg 82%).

Step 4: To a stirred suspension of 10,11-diazatricyclododeca-(2),1(5),3,6,8(10)-pentaen-9-one 5 (85 mg, 499.51 μmol) in acetonitrile (3 mL) at 0° C. was added N-bromosuccinimide (88.90 mg, 499.51 μmol, 42.33 μL). Then the cooling bath was removed and the reaction mixture stirred at 25° C. for 14 hrs. The reaction mixture was evaporated, quenched with saturated Na₂S203 solution, and extracted with ethyl acetate. The organic layer was washed with water followed by brine and dried over anhydrous sodium sulfate, concentrated, and purified by silica gel column chromatography (60% ethyl acetate in hexane) to afford 6-bromo-10,11-diazatricyclododeca-(2),1(4),3(6),5(7),8(10)-pentaen-9-one 6 as a yellowish solid (40 mg, 31%).

Step 5: To a solution of 6-bromo-10,11-diazatricyclododeca-(2),1(4),3(6),5(7),8(10)-pentaen-9-one 6 (1 eq) in THF (10 vol eq) at 0° C. is added NaH (5 eq) and stirred at this temp for 15 min before the addition of 3-bromopiperidine-2,6-dione 7 (1 eq). The reaction mixture is slowly heated to 60° C. and stirred at this temperature until completion of the reaction. A standard workup and purification using standard protocols affords 3-(6-bromo-2-oxopyrrolo[4,3,2-ij]isoquinolin-1(2H)-yl)piperidine-2,6-dione Compound 9.

### Example 9. Synthesis of 3-(6-bromo-2-oxopyrrolo[2,3,4-de]isoquinolin-1(2H)-yl)piperidine-2,6-dione (Compound 10)

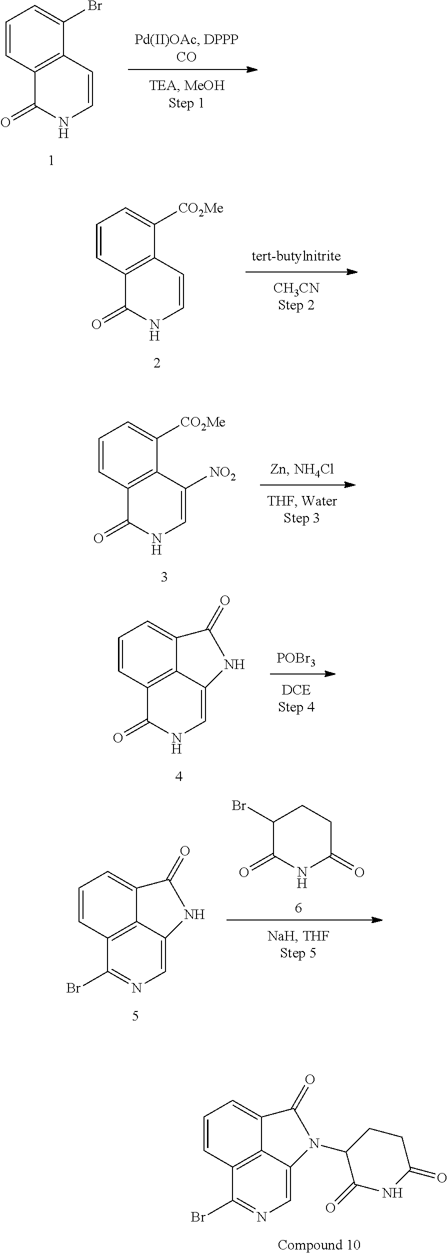

Step 1: A stirred solution of 5-bromo-2H-isoquinolin-1-one 1 (18 g, 80.34 mmol) and 1,3-bis(diphenylphosphino)propane (6.63 g, 16.07 mmol) in methanol (50.0 mL) was degassed with argon for 5 minutes, followed by addition of triethylamine, 99% (32.52 g, 321.35 mmol, 44.79 mL) and palladium(II) acetate (1.80 g, 8.03 mmol) into the reaction mixture. The resulting reaction mixture was heated in a Par autoclave in 80 psi of CO at 100° C. for 12 hr. The reaction mixture was filtered through celite, and the filtrate concentrated and purified by silica gel column chromatography (40% ethyl acetate in hexane) to afford methyl 1-oxo-2H-isoquinoline-5-carboxylate 2 (12 g, 54.92 mmol, 68.36% yield, 93% purity) as a grey colored solid.

Step 2: To a stirred solution of methyl 1-oxo-2H-isoquinoline-5-carboxylate 2 (7.7 g, 37.89 mmol) in acetonitrile (100 mL) was added tert-butylnitrite (15.63 g, 151.58 mmol, 18.03 mL). The reaction mixture was heated at 60° C. for 16 hours, then concentrated under reduced pressure. The crude material was treated with acetonitrile (20 ml), cooled to 0° C., stirred for 20 minutes and filtered. Solid residue was washed with ether and dried under reduced pressure to afford methyl 4-nitro-1-oxo-2H-isoquinoline-5-carboxylate 3 (4.5 g, 17.42 mmol, 45.97% yield, 96.07% purity) as a white solid.

Step 3: To a stirred solution of methyl 4-nitro-1-oxo-2H-isoquinoline-5-carboxylate 3 (2 g, 8.06 mmol) in THE (20 mL) and water (5 mL), zinc (526.93 mg, 8.06 mmol, 73.80 μL) and ammonium chloride (431.05 mg, 8.06 mmol, 281.73 μL) were added at room temperature. The mixture was then heated to 70° C. for 12 hr. After cooling to rt, the mixture was filtered through celite and the filtrate concentrated to afford crude 10,11-diazatricyclododeca-(2),1(4),3(6),5(7)-tetraene-8,9-dione 4 (800 mg, 3.44 mmol, 42.66% yield, 80% purity) as a yellow solid. Used in the next step without further purification.

Step 4: To a stirred solution of 10,11-diazatricyclododeca-(2),1(4),3(6),5(7)-tetraene-8,9-dione 4 (500 mg, 2.69 mmol) in DCE (40 mL) was added phosphoryl bromide (615.99 mg, 2.15 mmol, 218.43 μL) and the reaction mixture heated to 90° C. for 16 h. The reaction mixture was then cooled to RT, poured into ice water, basified with sodium bicarbonate, extracted with ethyl acetate, washed with brine, dried over anhydrous sodium sulfate, and concentrated under reduced pressure. The resulting crude was purified by silica column chromatography eluting with 20% ethyl acetate in hexane to afford 8-bromo-10,11-diazatricyclododeca-(2),1(4),3(6),5(7),8(10)-pentaen-9-one 5 (70 mg, 252.95 μmol, 9.42% yield, 90% purity) as a yellow solid.

Step 5: To a solution of 8-bromo-10,11-diazatricyclododeca-(2),1(4),3(6),5(7),8(10)-pentaen-9-one 5 (1 eq) in THE (10 vol eq) at 0° C. is added NaH (5 eq) and stirred at this temp for 15 min before the addition of 3-bromopiperidine-2,6-dione 6 (1 eq). The reaction mixture is slowly heated to 60° C. and stirred at this temperature until completion of the reaction. A standard workup and purification using standard protocols affords 3-(6-bromo-2-oxopyrrolo[2,3,4-de]isoquinolin-1(2H)-yl)piperidine-2,6-dione Compound 10.

### Example 10. Synthesis of afford 3-(6-bromo-2-oxopyrrolo[4,3,2-de]quinolin-1(2H)-yl)piperidine-2,6-dione (Compound 11)

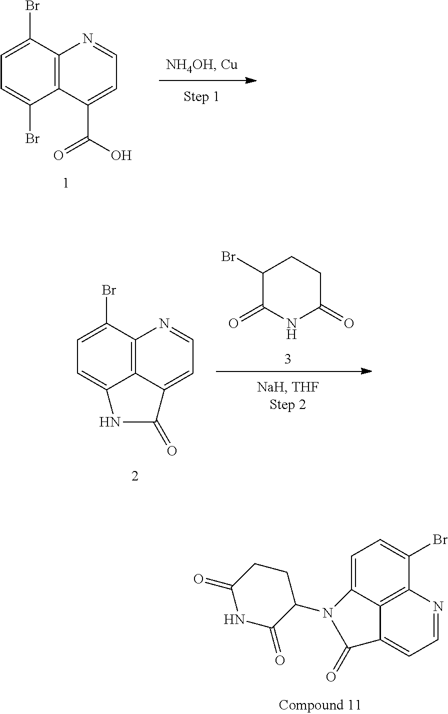

Step 1: To a stirred suspension of 5,8-dibromoquinoline-4-carboxylic acid 1 (CAS: 1603199-45-6) in ammonium hydroxide (28% solution) (100 eq), copper (4 eq) is added and the reaction mixture is stirred at 80° C. for 2 hr. The reaction mixture is cooled to RT and worked up and purified using standard protocols to afford 6-bromopyrrolo[4,3,2-de]quinolin-2(1H)-one 2 as the product.

Step 2: To a solution of 6-bromopyrrolo[4,3,2-de]quinolin-2(1H)-one 2 in THE (10 vol eq) at 0° C. is added NaH (5 eq) and stirred at this temp for 15 min before the addition of 3-bromopiperidine-2,6-dione 3 (1 eq). The reaction mixture is slowly heated to 60° C. and stirred at this temperature until completion of the reaction. A standard workup and purification using standard protocols affords 3-(6-bromo-2-oxopyrrolo[4,3,2-de]quinolin-1(2H)-yl)piperidine-2,6-dione Compound 11.

### Example 11. Synthesis of 3-(8-bromo-5-oxopyrrolo[2,3,4-de]quinolin-4(5H)-yl)piperidine-2,6-dione (Compound 12)

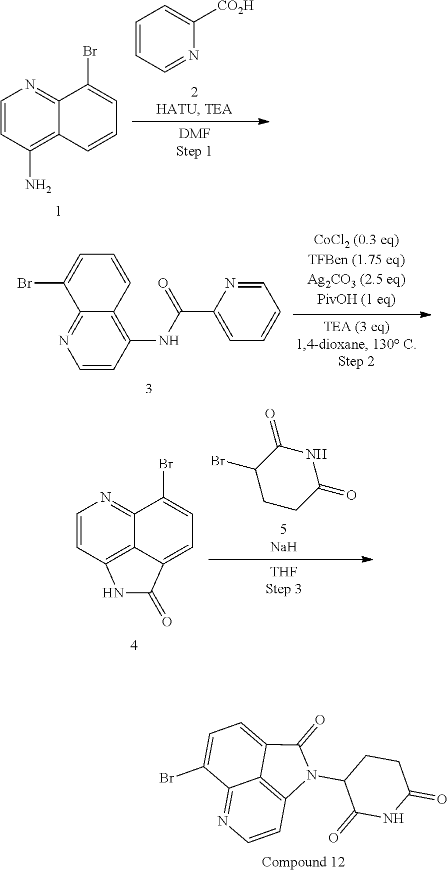 [SMILES: Nc1ccnc2c(Br)cccc12 ; O=C(O)c1ccccn1 ; O=C(Nc1ccnc2c(Br)cccc12)c1ccccn1 ; O=C1Nc2ccnc3c(Br)ccc1c23 ; O=C1CCC(Br)C(=O)N1 ; O=C1CCC(N2C(=O)c3ccc(Br)c4nccc2c34)C(=O)N1]

Step 1: To a stirred suspension of 8-bromoquinolin-4-amine 1 (CAS: 65340-75-2) in DMF (10 vol eq) is added Picolinic acid 2 (1 eq), TEA (3 eq) followed by HATU (1.1 eq) and the mixture is stirred at room temperature. Upon completion of the reaction the mixture is quenched, worked up, and purified using standard protocols to afford N-(8-bromoquinolin-4-yl)picolinamide 3.

Step 2: A suspension of N-(8-bromoquinolin-4-yl)picolinamide 3 (1 eq), CoCl₂ (0.3 eq), Ag₂CO₃ (2.5 eq), benzene-1,3,5-triyl triformate (TFBen, 1.75 eq), PivOH (1 eq) and TEA (3 eq) in 1,4-dioxane (10 vol eq) is heated at 130° C. for 20 h. Upon reaction completion the mixture is worked up and purified using standard protocols to afford 8-bromopyrrolo[2,3,4-de]quinolin-5(4H)-one 4. (According to procedures from Org. Lett. 2019, 21, 5694-5698.) Step 3: To a 8-bromopyrrolo[2,3,4-de]quinolin-5(4H)-one 4 in THE (10 vol eq) at 0° C. is added NaH (5 eq) and stirred at this temp for 15 min before the addition of 3-bromopiperidine-2,6-dione 5 (1 eq). The reaction mixture is slowly heated to 60° C. and stirred at this temperature until completion of the reaction. A standard workup and purification using standard protocols affords 3-(8-bromo-5-oxopyrrolo[2,3,4-de]quinolin-4(5H)-yl)piperidine-2,6-dione Compound 12.

### Example 12. Synthesis of 3-(5-bromo-2-oxopyrrolo[2,3,4-de]isoquinolin-1(2H)-yl)piperidine-2,6-dione (Compound 13)

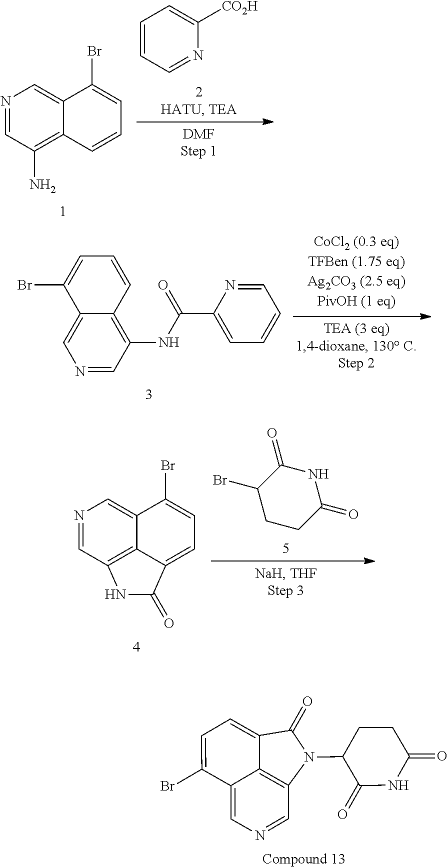 [SMILES: Nc1cncc2c(Br)cccc12 ; O=C(O)c1ccccn1 ; O=C(Nc1cncc2c(Br)cccc12)c1ccccn1 ; O=C1Nc2cncc3c(Br)ccc1c23 ; O=C1CCC(Br)C(=O)N1 ; O=C1CCC(N2C(=O)c3ccc(Br)c4cncc2c34)C(=O)N1]

Step 1: To a stirred suspension of 8-bromoisoquinolin-4-amine 1 (CAS: 1781091-48-2) in DMF (10 vol eq) is added picolinic acid 2 (1 eq), TEA (3 eq) followed by HATU (1.1 eq) and the mixture is stirred at room temperature. Upon completion of the reaction the mixture is quenched, worked up, and purified using standard protocols to afford N-(8-bromoisoquinolin-4-yl)picolinamide 3.

Step 2: A suspension of N-(8-bromoisoquinolin-4-yl)picolinamide 3 (1 eq), CoCl₂ (0.3 eq), Ag₂CO₃ (2.5 eq), benzene-1,3,5-triyl triformate (TFBen, 1.75 eq), PivOH (1 eq) and TEA (3 eq) in 1,4-dioxane (10 vol eq) is heated at 130° C. for 20 h. Upon reaction completion the mixture is worked up and purified using standard protocols to afford 5-bromopyrrolo[2,3,4-de]isoquinolin-2(1H)-one 4. (According to procedures from Org. Lett. 2019, 21, 5694-5698.) Step 3: To a 5-bromopyrrolo[2,3,4-de]isoquinolin-2(1H)-one 4 in THE (10 vol eq) at 0° C. is added NaH (5 eq) and stirred at this temp for 15 min before the addition of 3-bromopiperidine-2,6-dione 5 (1 eq). The reaction mixture is slowly heated to 60° C., and stirred at this temperature until completion of the reaction. A standard workup and purification using standard protocols affords 3-(5-bromo-2-oxopyrrolo[2,3,4-de]isoquinolin-1(2H)-yl)piperidine-2,6-dione Compound 13.

### Example 13. Synthesis of 3-(6-bromo-2-oxopyrrolo[4,3,2-de]isoquinolin-1(2H)-yl)piperidine-2,6-dione (Compound 14)

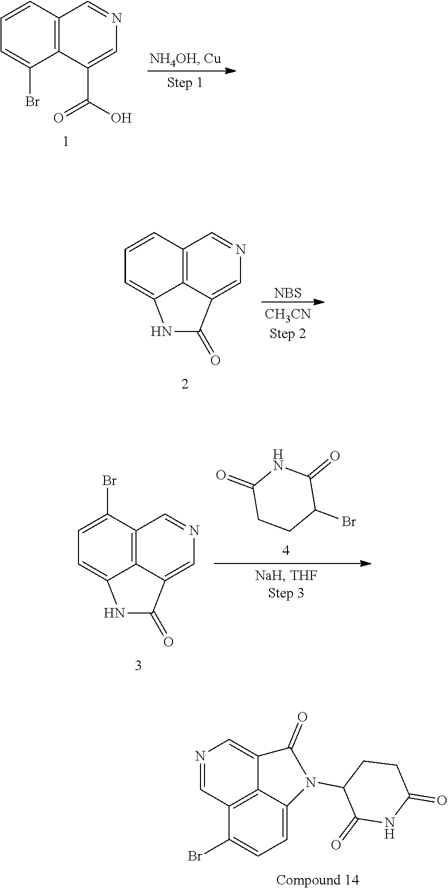

Step 1: To a stirred suspension of 5-bromoisoquinoline-4-carboxylic acid 1 (WO2012090177A2, 1 eq) in ammonium hydroxide (28% solution) (100 eq), copper (4 eq) is added and the reaction mixture is stirred at 80° C. for 2 hr. The reaction mixture is cooled to RT and worked up and purified using standard protocols to afford pyrrolo[4,3,2-de]isoquinolin-2(1H)-one 2.

Step 2: To a solution of pyrrolo[4,3,2-de]isoquinolin-2(1H)-one 2 (1 eq) in CH₃CN (10 vol) at 0° C. is added NBS (1 eq), the cooling bath is removed and the reaction mixture stirred at room temperature for 16 hours. A standard workup and purification using standard protocols to afford 6-bromopyrrolo[4,3,2-de]isoquinolin-2(1H)-one 3.

Step 3: To a solution 6-bromopyrrolo[4,3,2-de]isoquinolin-2(1H)-one (1 eq) in THE (10 vol eq) at 0° C. is added NaH (60% in mineral oil, 5 eq) and stirred at this temperature for 15 min before the addition of 3-bromopiperidine-2,6-dione 4 (1 eq). The reaction mixture is slowly heated to 60° C. and stirred at this temperature until completion of the reaction. A standard workup and purification using standard protocols affords 3-(6-bromo-2-oxopyrrolo[4,3,2-de]isoquinolin-1(2H)-yl)piperidine-2,6-dione Compound 14.

### Example 14. Synthesis of 3-(6-bromo-2-oxopyrrolo[2,3,4-ij]isoquinolin-1(2H)-yl)piperidine-2,6-dione (Compound 15)

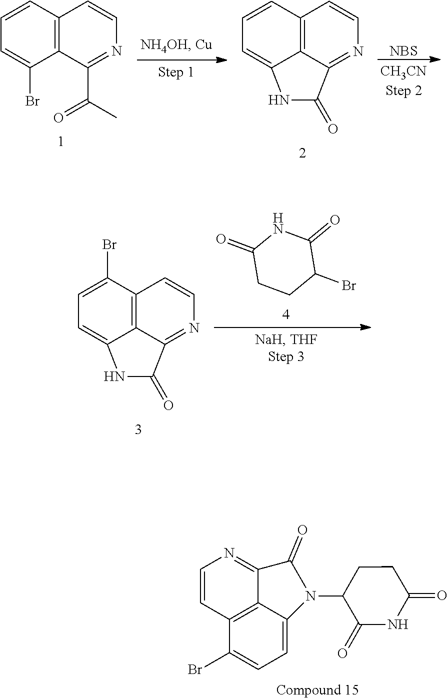

Step 1: To a stirred suspension of 8-bromoisoquinoline-1-carboxylic acid 1 (CAS #: 1256818-87-7, 1 eq) in ammonium hydroxide (28% solution) (100 eq), copper (4 eq) is added and the reaction mixture is stirred at 80° C. for 2 hr. The reaction mixture was cooled to RT and worked up and purified using standard protocols to afford pyrrolo[2,3,4-ij]isoquinolin-2(1H)-one 2.

Step 2: To a solution of pyrrolo[2,3,4-ij]isoquinolin-2(1H)-one 2 (1 eq) in CH₃CN (10 vol) at 0° C. is added NBS (1 eq), the cooling bath is removed and the reaction mixture stirred at room temperature for 16 hours. A standard workup and purification using standard protocols affords 6-bromopyrrolo[2,3,4-ij]isoquinolin-2(1H)-one 3.

Step 3: To a solution 6-bromopyrrolo[2,3,4-ij]isoquinolin-2(1H)-one 3 (1 eq) in THE (10 vol eq) at 0° C. is added NaH (60% in mineral oil, 5 eq) and stirred at this temperature for 15 min before the addition of 3-bromopiperidine-2,6-dione 4 (1 eq). The reaction mixture is slowly heated to 60° C. and stirred at this temperature until completion of the reaction. A standard workup and purification using standard protocols affords 3-(6-bromo-2-oxopyrrolo[2,3,4-ij]isoquinolin-1(2H)-yl)piperidine-2,6-dione Compound 15.

### Example 15. Synthesis of 3-(3-bromo-8-oxopyrrolo[4,3,2-de]phthalazin-7(8H)-yl)piperidine-2,6-dione (Compound 16)

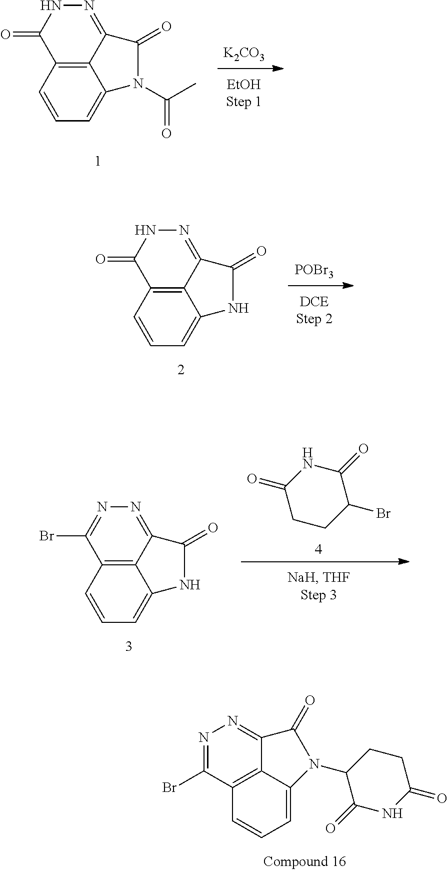

Step 1: To a solution of 7-acetyl-2,7-dihydropyrrolo[4,3,2-de]phthalazine-3,8-dione 1 (Heterocycles (1981), 16(1), 21-4, 1 eq) in EtOH (10 vol eq) is added potassium carbonate (3 eq) and the reaction mixture stirred while heating from room temperature to 50° C. Standard workup and purification using standard protocols affords 2,7-dihydropyrrolo[4,3,2-de]phthalazine-3,8-dione 2.

Step 2: To a solution of 2,7-dihydropyrrolo[4,3,2-de]phthalazine-3,8-dione 2 in DCE (10 vol eq) is added POBr₃ (1 eq) and the reaction stirred at 90° C. for 16 h. A standard workup and purification using standard protocols affords 3-bromopyrrolo[4,3,2-de]phthalazin-8(7H)-one 3.

Step 3: To a solution of 3-bromopyrrolo[4,3,2-de]phthalazin-8(7H)-one 3 (1 eq) in THE (10 vol eq) at 0° C. is added NaH (5 eq) and stirred at this temp for 15 min before the addition of 3-bromopiperidine-2,6-dione 4 (1 eq). The reaction mixture is slowly heated to 60° C. and stirred at this temperature until completion of the reaction. A standard workup and purification using standard protocols affords 3-(3-bromo-8-oxopyrrolo[4,3,2-de]phthalazin-7(8H)-yl)piperidine-2,6-dione Compound 16.

### Example 16. Synthesis of 3-(6-bromo-2-oxopyrrolo[4,3,2-de]quinazolin-1(2H)-yl)piperidine-2,6-dione (Compound 17)

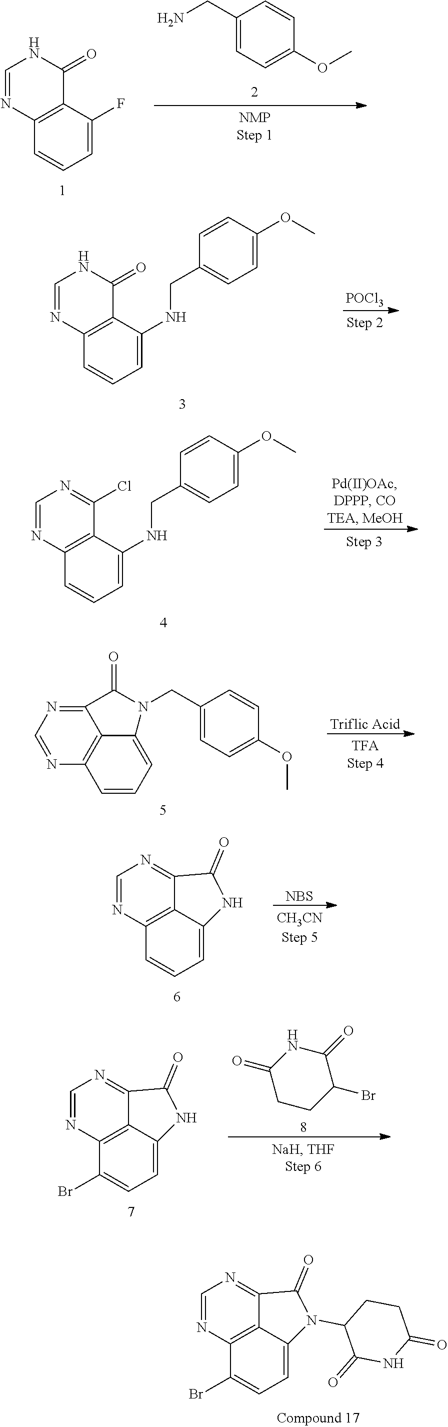

Step 1: A solution of 5-fluoro-4(1H)-quinazolinone 1 (CAS #436-72-6, 1 eq) and 4-methylbenzylamine 2 (5 eq) in NMP is heated 100° C. until completion of the reaction. A standard work up and purification using standard protocols affords 5-((4-methoxybenzyl)amino)quinazolin-4(3H)-one 3.

Step 2: To a solution of 5-((4-methoxybenzyl)amino)quinazolin-4(3H)-one 3 (1 eq) in a toluene (10 vol eq) is added POCl₃ (1 eq) and the reaction mixture is heated to 100° C. until completion of the reaction. A standard workup and purification using standard protocols affords 4-chloro-N-(4-methoxybenzyl)quinazolin-5-amine 4.

Step 3: To a solution of 4-chloro-N-(4-methoxybenzyl)quinazolin-5-amine 4 (1 ea) in MeOH (10 vol eq) is added TEA (4 eq) then the solution is purged with argon for 10 min. DPPP (0.2 eq) and palladium(II) acetate (0.1 eq) is added and the reaction mixture shaken in a parr-autoclave at 100° C. under an atmosphere of 70 psi of carbon monoxide until the reaction is deemed complete. A standard workup and purification using standard protocols affords 1-(4-methoxybenzyl)pyrrolo[4,3,2-de]quinazolin-2(1H)-one 5.

Step 4: To a cooled solution of produce 1-(4-methoxybenzyl)pyrrolo[4,3,2-de]quinazolin-2(1H)-one 5 in TFA (12 vol eq) is added triflic acid (8 eq) and the reaction mixture is stirred at room temperature until the reaction is complete. A standard workup and purification using standard protocols affords pyrrolo[4,3,2-de]quinazolin-2(1H)-one 6.

Step 5: To a mixture of pyrrolo[4,3,2-de]quinazolin-2(1H)-one 6 (1 eq) in CH₃CN (10 vol eq) is added NBS (1 eq) at 0° C., the cooling bath removed and the reaction mixture stirred at room temperature until the reaction is deemed complete. A standard workup and purification using standard protocols affords 6-bromopyrrolo[4,3,2-de]quinazolin-2(1H)-one 7.

Step 6: To a solution of 6-bromopyrrolo[4,3,2-de]quinazolin-2(1H)-one 7 (1 eq) in THE (10 vol eq) at 0° C. is added NaH (5 eq) and stirred at this temp for 15 min before the addition of 3-bromopiperidine-2,6-dione (1 eq). The reaction mixture is slowly heated to 60° C. and stirred at this temperature until completion of the reaction. A standard workup and purification using standard protocols affords 3-(6-bromo-2-oxopyrrolo[4,3,2-de]quinazolin-1(2H)-yl)piperidine-2,6-dione Compound 17.

### Example 17. Synthesis of 3-(8-bromo-5-oxopyrrolo[2,3,4-de]quinazolin-4(5H)-yl)piperidine-2,6-dione (Compound 18)

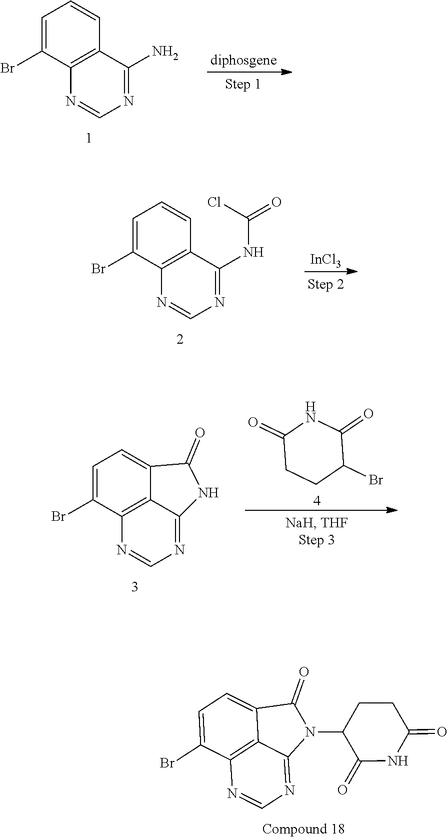

Step 1: To a solution of 8-bromo-4-quinazolinamine 1 (CAS #1260657-19-9, 1 eq) in dichloroethane:pyridine (10:1) at 0° C. is added diphosgene (1.1-1.5 eq) and the reaction stirred at this temp for 2 hours, followed by slowly increasing the temperature to 50° C. then maintaining at this temperature for 2 hours. The reaction mixture is quenched with 1N HCl and standard work up and purification affords (8-bromoquinazolin-4-yl)carbamic chloride 2.

Step 2: To a solution of (8-bromoquinazolin-4-yl)carbamic chloride 2 in dichloroethane at 0° C. is added indium trichloride (1.1-5 eq). The reaction mixture heated to reflux and maintained at this temperature until completion of the reaction. The cooled reaction mixture is then subjected to a standard work up and purification to afford 8-bromopyrrolo[2,3,4-de]quinazolin-5(4H)-one 3.

Step 3: To a solution of 8-bromopyrrolo[2,3,4-de]quinazolin-5(4H)-one 3 in THE at 0° C. is added NaH (60% dispersion in mineral oil, 10-15 eq) in portions. The cooling bath is removed and the reaction mixture is stirred at room temperature for 1 hours. The reaction mixture is cooled to 0° C. and 3-bromo-glutarimide 4 (5-8 eq) is added in portions. After addition, the cooling bath removed and the reaction is slowly heated to 70° C. and stirred until the reaction is judged complete. Standard workup and purification using standard protocols affords 3-(8-bromo-5-oxopyrrolo[2,3,4-de]quinazolin-4(5H)-yl)piperidine-2,6-dione Compound 18.

### Example 18. Synthesis of 3-(3-bromo-2-methyl-7-oxo-2,7-dihydro-6H-pyrrolo[4,3,2-cd]indazol-6-yl)piperidine-2,6-dione (Compound 19) and 3-(3-bromo-2-methyl-6-oxo-2,6-dihydro-7H-pyrrolo[2,3,4-cd]indazol-7-yl)piperidine-2,6-dione (Compound 20)

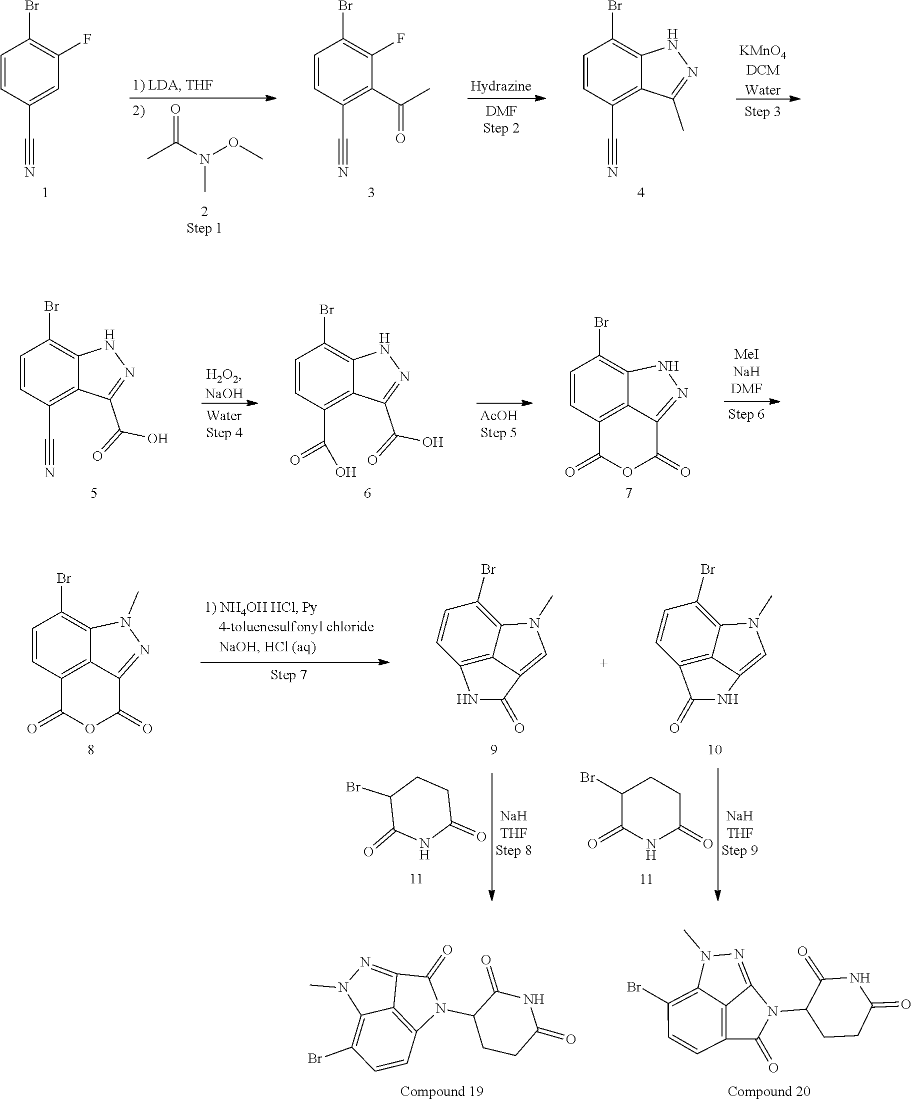

Step 1: To a solution of commercially available 4-bromo-3-fluorobenzonitrile 1 (Cas #: 133059-44-6, 1 eq.) in THE at −78° C. is added a dropwise solution of LDA (2M in THF, 1.1 eq) and stirred at this temperature for 1-3 hours. At this time a solution of N-methoxy-N-methylacetamide 2 (1.2 eq) in THF is added dropwise, the cooling bath is removed, and the reaction mixture is stirred for 1-24 additional hours. Isolation and purification using standard procedures affords 2-acetyl-4-bromo-3-fluorobenzonitrile 3.

Step 2: To a solution of 2-acetyl-4-bromo-3-fluorobenzonitrile 3 (1 eq), in DMF at 0° C. is added hydrazine (1.1 eq) dropwise. Then the cooling bath is removed and the reaction is allowed to stir at room temperature for an additional 1-24 hours. Isolation and purification using standard procedures affords 7-bromo-3-methyl-1H-indazole-4-carbonitrile 4.

Step 3: To a solution of 7-bromo-3-methyl-1H-indazole-4-carbonitrile 4 (1 eq) in a mixture of DCM and water is added KMnO₄ (10 eq) and stirred at room temperature to reflux for 1-24 hours. Isolation and purification using standard protocols affords 7-bromo-4-cyano-1H-indazole-3-carboxylic acid 5.

Step 4: To a solution of 7-bromo-4-cyano-1H-indazole-3-carboxylic acid 5 (1 eq) in 4:1 water:hydrogen peroxide is added 20 eq of NaOH and the reaction mixture refluxed for 1-24 hours. Isolation and purification using standard protocols affords 7-bromo-1H-indazole-3,4-dicarboxylic acid 6.

Step 5: A mixture of 7-bromo-1H-indazole-3,4-dicarboxylic acid 6 (1 eq) in AcOH (10 vol eq.) is heated at 100° C. until completion of the reaction. Standard workup and purification protocols afford 8-bromo-3H-pyrano[3,4,5-cd]indazole-3,5(1H)-dione 7.

Step 6: To a cooled solution of 8-bromo-3H-pyrano[3,4,5-cd]indazole-3,5(1H)-dione 7 (1 eq) in DMF is added NaH (60% in oil, 2 eq) and the reaction mixture stirred at this temp for 10 min before the addition of Mel (1.1 eq). The cooling bath is removed and the reaction mixture stirred until completion of the reaction. A standard workup and purification using standard protocols affords 8-bromo-1-methyl-3H-pyrano[3,4,5-cd]indazole-3,5(1H)-dione 8.

Step 7: A solution of 8-bromo-1-methyl-3H-pyrano[3,4,5-cd]indazole-3,5(1H)-dione 8 (1 eq, 18.05 mmol) and hydroxylamine hydrochloride (1 eq, 1.25 g, 18.05 mmol, 750.92 μL) in pyridine (10 vol eq.) is heated at reflux for 5 h, followed by cooling to 80° C. and the addition of 4-toluenesulfonyl chloride (2 eq). After addition, the temperature is raised and the reaction stirred at reflux for 5 h, followed by cooling. The reaction mixture is poured into water and extracted with EtOAc (3×). The organic layers are combined, washed with water, sat. aq. NaHCO₃, brine, and dried over anhydrous Na₂SO₄, then filtered and evaporated to dryness. To a stirred solution of the residue in EtOH (10 vol eq) and water (10 vol eq) is added 1M aqueous sodium hydroxide (10 eq) dropwise. Thereafter, the mixture is stirred at reflux for 3 h while distilling off the ethanol. After completion of the reaction, the reaction mixture is cooled to 75° C., and hydrochloric acid, 36% w/w aq. soln. (10 vol eq) is added dropwise. Standard work up and purification followed by separation of regioisomers affords 3-bromo-2-methyl-2,6-dihydro-7H-pyrrolo[4,3,2-cd]indazol-7-one 9 and 3-bromo-2-methyl-2,7-dihydro-6H-pyrrolo[2,3,4-cd]indazol-6-one 10.

Step 8: To a solution of 3-bromo-2-methyl-2,6-dihydro-7H-pyrrolo[4,3,2-cd]indazol-7-one 9 in THE at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide 11 (5-8 eq) is added in portions before the cooling bath is once again removed and the reaction slowly heated to 70° C. until the reaction is judged complete. A standard workup and purification using standard protocols affords 3-(3-bromo-2-methyl-7-oxo-2,7-dihydro-6H-pyrrolo[4,3,2-cd]indazol-6-yl)piperidine-2,6-dione Compound 19.

Step 9: To a solution of 3-bromo-2-methyl-2,7-dihydro-6H-pyrrolo[2,3,4-cd]indazol-6-one in THE at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide 11 (5-8 eq) is added in portions before the cooling bath is once again removed and the reaction slowly heated to 70° C. until the reaction is judged complete. A standard workup and purification using standard protocols affords 3-(3-bromo-2-methyl-6-oxo-2,6-dihydro-7H-pyrrolo[2,3,4-cd]indazol-7-yl)piperidine-2,6-dione Compound 20.

### Example 19. Synthesis of 3-(6-bromo-2-oxo-3,4-dihydro-5-oxa-1,2a-diazaacenaphthylen-1(2H)-yl)piperidine-2,6-dione (Compound 21)

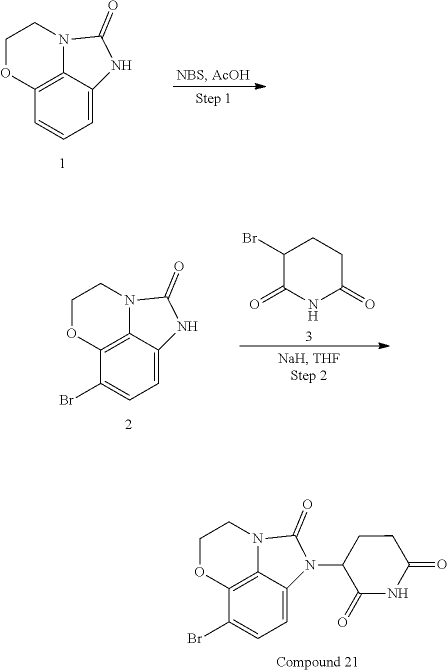

Step 1: To a solution of 3,4-dihydro-5-oxa-1,2a-diazaacenaphthylen-2(1H)-one 1 (1 eq) (CAS #: 1267075-60-4) in acetic acid is added N-bromosuccinimide (1.2 eq) at room temperature. The reaction mixture is stirred at rt until judged complete. The reaction mixture is then subjected to a standard work up and purification to afford 6-bromo-3,4-dihydro-5-oxa-1,2a-diazaacenaphthylen-2(1H)-one 2.

Step 2: in THE at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide 3 (5-8 eq) is added in portions before the cooling bath is once again removed and the reaction slowly heated to 70° C. until the reaction is judged complete. A standard workup and purification using standard protocols affords 3-(6-bromo-2-oxo-3,4-dihydro-5-oxa-1,2a-diazaacenaphthylen-1(2H)-yl)piperidine-2,6-dione Compound 21.

### Example 20. Synthesis of 3-(7-bromo-2-oxo-5,6-dihydro-4H-imidazo[4,5,1-ij]quinolin-1(2H)-yl)piperidine-2,6-dione (Compound 22)

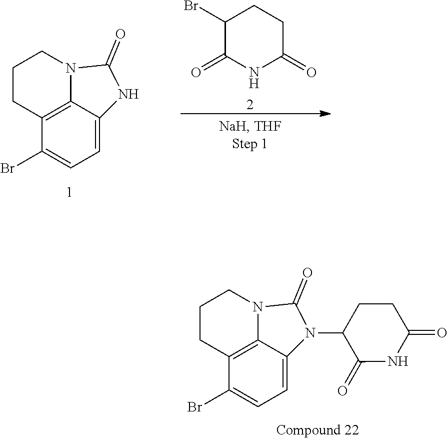

Step 1: To a solution of 7-bromo-5,6-dihydro-4H-imidazo[4,5,1-ij]quinolin-2(1H)-one 1 (1 eq) (CAS #: 1609453-63-5) in THE at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide 2 (5-8 eq) is added in portions before the cooling bath is once again removed and the reaction slowly heated to 70° C. until the reaction is judged complete. A standard workup and purification using standard protocols affords 3-(7-bromo-2-oxo-5,6-dihydro-4H-imidazo[4,5,1-ij]quinolin-1(2H)-yl)piperidine-2,6-dione Compound 22.

### Example 21. Synthesis of 3-(5-bromo-1-oxo-6,7-dihydroimidazo[4,5,1-hi]indol-2(1H)-yl)piperidine-2,6-dione (Compound 23)

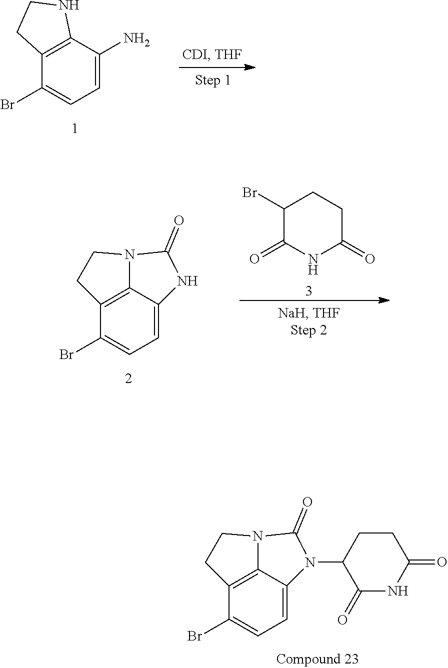

Step 1: To a solution of 4-bromoindolin-7-amine 1 (1 eq) (CAS #: 1783558-27-9) in THE is added 1,1′-carbonyldiimidazole (1.2 eq) at room temperature. The reaction mixture is heated to reflux until judged complete. The cooled reaction mixture is then subjected to a standard work up and purification to afford 5-bromo-6,7-dihydroimidazo[4,5,1-hi]indol-1(2H)-one 2.

Step 2: To a solution of 5-bromo-6,7-dihydroimidazo[4,5,1-hi]indol-1(2H)-one 2 (1 eq) in THF at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide 3 (5-8 eq) is added in portions before the cooling bath is once again removed and the reaction slowly heated to 70° C. until the reaction is judged complete. A standard workup and purification using standard protocols affords 3-(5-bromo-1-oxo-6,7-dihydroimidazo[4,5,1-hi]indol-2(1H)-yl)piperidine-2,6-dione Compound 23.

### Example 22. Synthesis of 3-(7-bromo-2-oxo-4H-imidazo[4,5,1-ij]quinolin-1(2H)-yl)piperidine-2,6-dione (Compound 24)

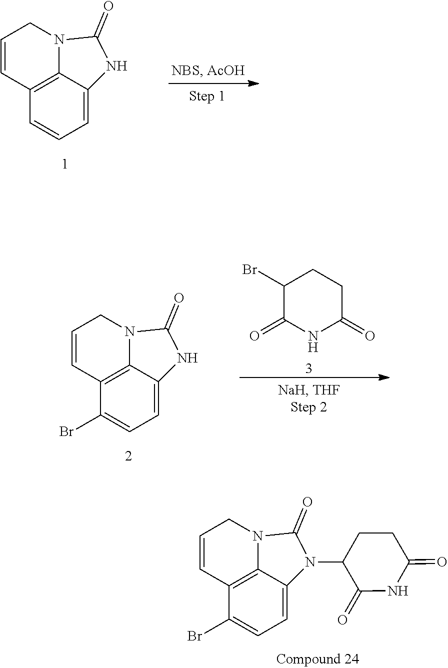

3-(7-bromo-2-oxo-4H-imidazo[4,5,1-ij]quinolin-1(2H)-yl)piperidine-2,6-dione can be prepared in a similar manor to Example 19, Compound 21, except replacing 3,4-dihydro-5-oxa-1,2a-diazaacenaphthylen-2(1H)-one with 4H-Imidazo[4,5,1-ij]quinolin-2(1H)-one (CAS #83848-83-3).

### Example 23. Synthesis of 3-(5-bromo-1-oxo-2,9a-diazabenzo[cd]azulen-2(1H)-yl)piperidine-2,6-dione (Compound 25)

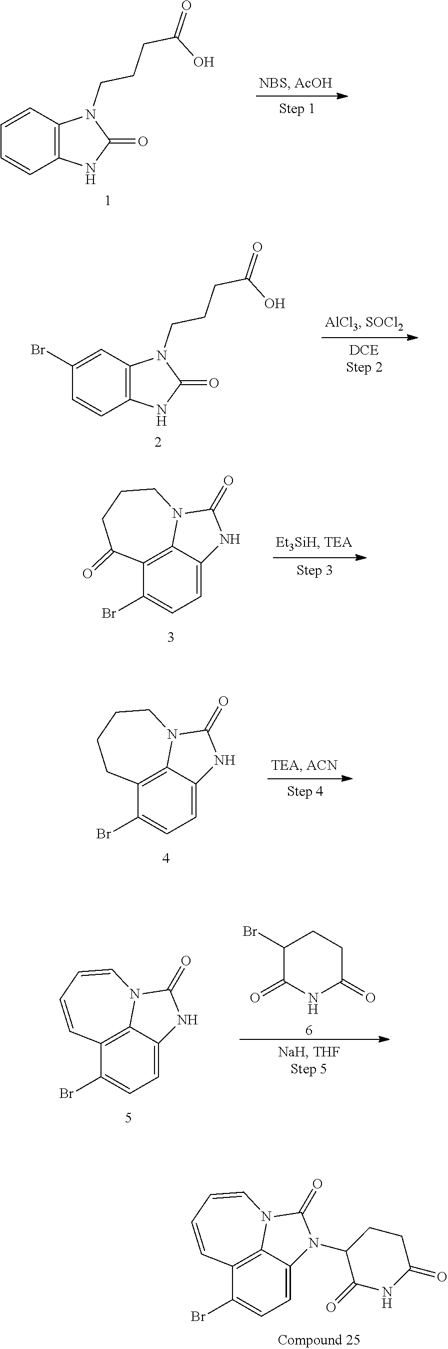

Step 1: To a solution of 4-(2-oxo-2,3-dihydro-1H-benzo[d]imidazol-1-yl)butanoic acid 1 (1 eq) (CAS #: 3273-68-5) in acetic acid is added N-Bromosuccinimide (1.2 eq) at room temperature. The reaction mixture is stirred at RT until judged complete. The reaction mixture is then subject to a standard work up and purification to afford 4-(6-bromo-2-oxo-2,3-dihydro-1H-benzo[d]imidazol-1-yl)butanoic acid 2.

Step 2: To a solution of 4-(6-bromo-2-oxo-2,3-dihydro-1H-benzo[d]imidazol-1-yl)butanoic acid 2 (1 eq) in dichloromethane is added thionyl chloride (2 eq) and the reaction mixture is stirred at room temperature for 2 hours. The mixture is concentrated in vacuo and to the residue is added dichloroethane and aluminum chloride (3 eq), added portionwise. The reaction mixture is stirred at rt to reflux until judged complete. The reaction mixture is then subject to a standard work up and purification to afford 5-bromo-8,9-dihydro-2,9a-diazabenzo[cd]azulene-1,6(2H,7H)-dione 3.

Step 3: To a solution TFA solution of 5-bromo-8,9-dihydro-2,9a-diazabenzo[cd]azulene-1,6(2H,7H)-dione 3 (1 eq) at 0°, Triethylsilane (1.2 eq) is added slowly and the solution is stirred at 0° until judged complete. The reaction mixture is then subject to a standard work up and purification to afford 5-bromo-6,7,8,9-tetrahydro-2,9a-diazabenzo[cd]azulen-1(2H)-one 4.

Step 4: To a solution of 5-bromo-6,7,8,9-tetrahydro-2,9a-diazabenzo[cd]azulen-1(2H)-one 4 (1 eq) in acetonitrile is added triethylamine (5 eq). The reaction mixture is stirred at RT to reflux until judged complete. The reaction mixture is then subject to a standard work up and purification to afford 5-bromo-2,9a-diazabenzo[cd]azulen-1(2H)-one 5.

Step 5: To a solution of 5-bromo-2,9a-diazabenzo[cd]azulen-1(2H)-one 5 (1 eq) in THE at 0° C. is added NaH (60% dispersion in mineral oil, 10-15 eq) in portions, the cooling bath is removed and the reaction mixture is stirred at this temperature for 1 hour. The reaction mixture is cooled to 0° C., 3-bromo-glutarimide 6 (5-8 eq) is added in portions, the cooling bath removed, and slowly heated to 70° C. until the reaction is judged complete. Standard workup and purification using standard protocols to afford 3-(5-bromo-1-oxo-2,9a-diazabenzo[cd]azulen-2(1H)-yl)piperidine-2,6-dione Compound 25.

### Example 24. Synthesis of 3-(5-bromo-7,8-dihydro-6H-pyrazolo[4,5,1-ij]quinolin-2-yl)piperidine-2,6-dione (Compound 26)

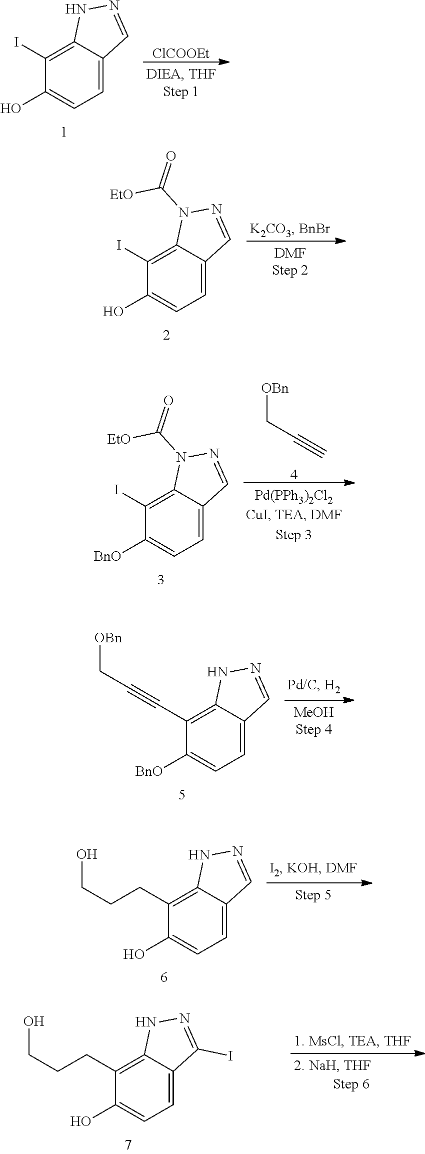

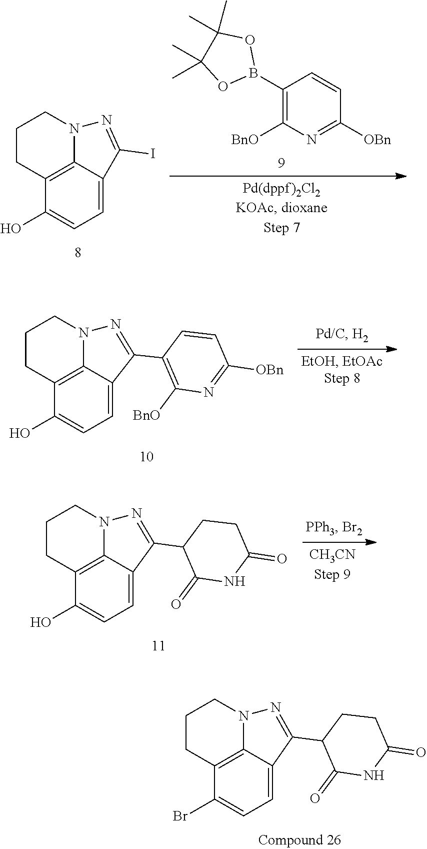 [SMILES: Oc1ccc2c(I)nn3c2c1CCC3 ; CC1(C)OB(c2ccc(OCc3ccccc3)nc2OCc2ccccc2)OC1(C)C ; Oc1ccc2c(-c3ccc(OCc4ccccc4)nc3OCc3ccccc3)nn3c2c1CCC3 ; O=C1CCC(c2nn3c4c(c(O)ccc24)CCC3)C(=O)N1 ; O=C1CCC(c2nn3c4c(c(Br)ccc24)CCC3)C(=O)N1]

Step 1: To a solution of 7-iodo-1H-indazol-6-ol 1 (1 eq) (CAS #: 1190314-62-5) in THE is added DIEA (1.2 eq), followed by ethyl chloroformate (1.1 eq) at 0° C. The reaction mixture is stirred at RT until judged complete. The reaction mixture is then subjected to a standard work up and purification to afford ethyl 6-hydroxy-7-iodo-1H-indazole-1-carboxylate 2.

Step 2: To a solution of ethyl 6-hydroxy-7-iodo-1H-indazole-1-carboxylate 2 (1 eq) in DMF is added potassium carbonate (1.5 eq), followed by benzyl bromide (1.1 eq) at 0° C. The reaction mixture is stirred at rt until judged complete. The reaction mixture is then subjected to a standard workup and purification to afford ethyl 6-(benzyloxy)-7-iodo-1H-indazole-1-carboxylate 3.

Step 3: A solution of ethyl 6-(benzyloxy)-7-iodo-1H-indazole-1-carboxylate 3 (1 eq) and benzyl propargyl ether 4 (1.5 eq) (CAS #: 4039-82-1) dissolved in DMF and TEA (3 eq) is degassed with Ar. Pd(PPh₃)₂Cl₂ (0.1 eq) and copper(I) iodide (0.1 eq) are added and the mixture is sealed and heated at 80° C. in microwave until judged complete. The reaction mixture is then subjected to a standard work up and purification to afford 6-(benzyloxy)-7-(3-(benzyloxy)prop-1-yn-1-yl)-1H-indazole 5.

Step 4: To a solution of 6-(benzyloxy)-7-(3-(benzyloxy)prop-1-yn-1-yl)-1H-indazole 5 (1 eq) in MeOH is added Pd/C (10%, 10 eq) under N₂ atmosphere. The suspension is degassed and purged with H₂ 3 times. The mixture is stirred under H₂ (15 psi) at RT until judged complete. The reaction mixture is then subjected to a standard work up and purification to afford 7-(3-hydroxypropyl)-1H-indazol-6-ol 6.

Step 5: To a solution of 7-(3-hydroxypropyl)-1H-indazol-6-ol 6 (1 eq) in DMF is added KOH (3 eq) and 12 (1.5 eq). The mixture is stirred at RT until judged complete. The reaction mixture is then subjected to a standard work up and purification to afford 7-(3-hydroxypropyl)-3-iodo-1H-indazol-6-ol 7.

Step 6: To a solution of 7-(3-hydroxypropyl)-3-iodo-1H-indazol-6-ol 7 (1 eq) in THE is added TEA (2 eq), followed by methanesulfonyl chloride (1.2 eq). The mixture is stirred at RT until judged complete. Solvent is removed and the residue is dissolved in THE and cooled to 0° C. NaH (60% in mineral oil, 2.2 eq) is then added portionwise and the mixture is stirred at RT until judged complete. The reaction mixture is then subjected to a standard work up and purification to afford 2-iodo-7,8-dihydro-6H-pyrazolo[4,5,1-ij]quinolin-5-ol 8.

Step 7: To a solution of 2,6-bis(benzyloxy)-3-(4,4,5,5-tetramethyl-1,3,2-dioxaborolan-2-yl)pyridine 9 (1 eq), 2-iodo-7,8-dihydro-6H-pyrazolo[4,5,1-ij]quinolin-5-ol 8 (1 eq), and Cs₂CO₃ (3 eq) in dioxane and H₂O (v/v 4:1) is added Pd(dppf)Cl₂ (0.1 eq). The reaction mixture is degassed with argon then stirred at 100° C. until judged complete. The reaction mixture is then subjected to a standard work up and purification to afford 2-(2,6-bis(benzyloxy)pyridin-3-yl)-7,8-dihydro-6H-pyrazolo[4,5,1-ij]quinolin-5-ol 10.

Step 8: To a solution of 2-(2,6-bis(benzyloxy)pyridin-3-yl)-7,8-dihydro-6H-pyrazolo[4,5,1-ij]quinolin-5-ol 10 (1 eq) in EtOH and EtOAc (v/v 1:1) is added Pd/C (10%, 10 eq) under N₂ atmosphere. The suspension is degassed and purged with H₂ 3 times. The mixture is stirred under H₂ (15 psi) at rt until judged complete. The reaction mixture is then subjected to a standard work up and purification to afford 3-(5-hydroxy-7,8-dihydro-6H-pyrazolo[4,5,1-ij]quinolin-2-yl)piperidine-2,6-dione 11.

Step 9: To a solution of 3-(5-hydroxy-7,8-dihydro-6H-pyrazolo[4,5,1-ij]quinolin-2-yl)piperidine-2,6-dione 11 (1 eq) in acetonitrile is added triphenylphosphine (1.3 eq) and bromine (2 eq). The reaction mixture is heated under reflux until judged complete. The reaction mixture is then subjected to a standard work up and purification to afford 3-(5-bromo-7,8-dihydro-6H-pyrazolo[4,5,1-ij]quinolin-2-yl)piperidine-2,6-dione Compound 26.

### Example 25. Synthesis of 3-(5-bromo-2-thioxobenzo[cd]indol-1(2H)-yl)piperidine-2,6-dione (Compound 27)

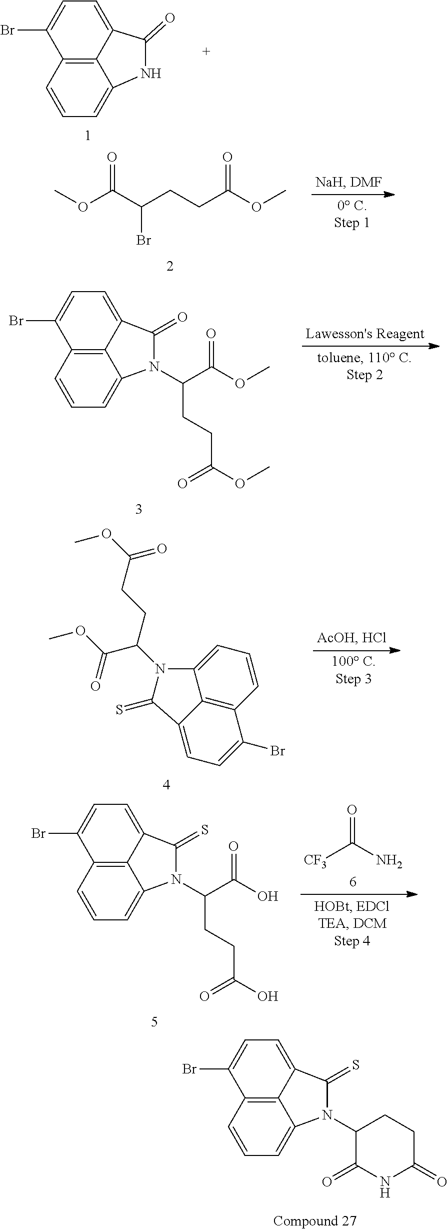

Step 1: To a solution of 5-bromobenzo[cd]indol-2(1H)-one 1 dissolved in anhydrous dimethylformamide is added a solution of sodium hydride 60% in mineral oil (1.3 eq). The mixture is stirred at room temperature for 1 hr. To the mixture is added dimethyl 2-bromopentanedioate 2 (CAS: 760-94-1, 1 eq). The resulting mixture is stirred at room temperature for 18 hr. The reaction is subjected to standard workup and purification using standard protocols to afford dimethyl 2-(5-bromo-2-oxobenzo[cd]indol-1(2H)-yl)pentanedioate 3. (Similarly described in WO2007056281)

Step 2: A solution of dimethyl 2-(5-bromo-2-oxobenzo[cd]indol-1(2H)-yl)pentanedioate 3 and Lawesson's reagent (CAS: 19172-47-5, 1 eq) dissolved in toluene is stirred at 110° C. for 10 hr. After the reaction is judged complete, the solvent is evaporated and the resulting crude material purified using standard protocols to afford dimethyl 2-(5-bromo-2-thioxobenzo[cd]indol-1(2H)-yl)pentanedioate 4. (Similarly described in WO 2005/028436 A2)

Step 3: A solution of dimethyl 2-(5-bromo-2-thioxobenzo[cd]indol-1(2H)-yl)pentanedioate 4, glacial acetic acid, and concentrated HCl (1:1) is stirred at 100° C. for 2.5 h. The reaction is subjected to standard workup, and purified using standard protocols to afford 2-(5-bromo-2-thioxobenzo[cd]indol-1(2H)-yl)pentanedioic acid 5. (Similarly described in WO 2005/028436 A2) Step 4: A mixture of 2-(5-bromo-2-thioxobenzo[cd]indol-1(2H)-yl)pentanedioic acid 5, trifluoroacetamide (CAS: 354-38-1, 1.8 eq), HOBt (3.9 eq), EDCI (3.9 eq) and triethylamine (5.5 eq) in CH₂Cl₂ is stirred at ambient temperature for 3 days. The reaction is subjected to standard work up, and purified using standard protocols to afford 3-(5-bromo-2-thioxobenzo[cd]indol-1(2H)-yl)piperidine-2,6-dione Compound 27. (Similarly described in WO 2005/028436 A2)

### Example 26. Synthesis of 3-(6-bromo-1H-benzo[de]isoquinolin-2(3H)-yl)piperidine-2,6-dione (Compound 28)

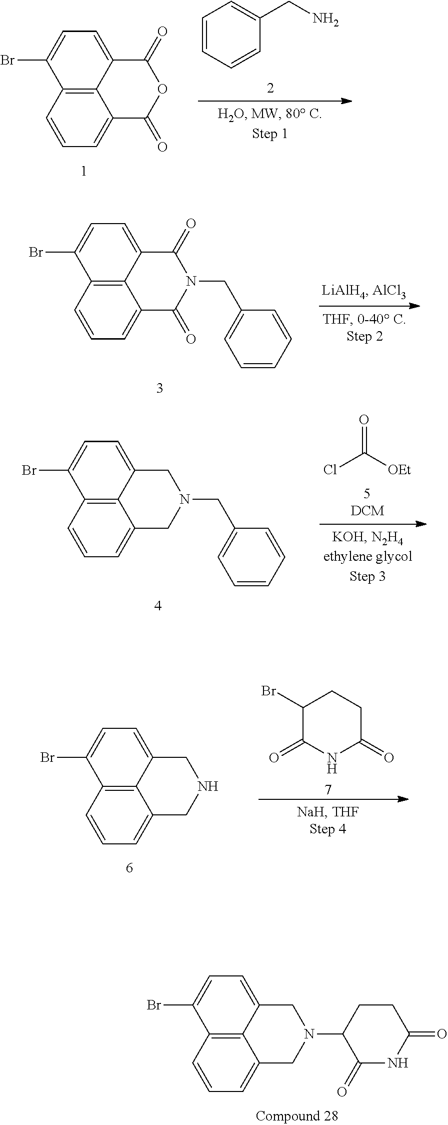

Step 1: A mixture of benzyl amine 2 (1.2 mmol, CAS: 100-46-9), water (10 mL), and 4-bromo-1,8-naphthalic anhydride 1 (1 mmol, CAS: 81-86-7) is mixed together in a sealed and pressurized tube and reacted at 450 W and 80° C. under microwave irradiation for a few minutes. After the reaction, the mixture is filtered to afford 2-benzyl-6-bromo-1H-benzo[de]isoquinoline-1,3(2H)-dione 3 (Yield: 95%). (As described in Synthetic Communications (2012), 42(20), 3042-3052).

Step 2: A solution of anhydrous aluminum chloride (4.0 mmol) and LiAlH₄ (4.0 mmol) is added to cold, dry THF (ice bath) with stirring. After removal of the ice bath, 2-benzyl-6-bromo-1H-benzo[de]isoquinoline-1,3(2H)-dione 3 (1.0 mmol) is added in small portions. The mixture is stirred at 40° C. for 5.5 hours and then at RT for 10 hours. The reaction is subjected to standard work up and purified using standard protocols to afford 2-benzyl-6-bromo-2,3-dihydro-1H-benzo[de]isoquinoline 4. (Similarly described in Journal of the American Chemical Society (2003), 125(19), 5786-5791).

Step 3: To a solution of ethyl chloroformate 5 (21 mmol), a solution of 2-benzyl-6-bromo-2,3-dihydro-1H-benzo[de]isoquinoline 4 (16 mmol) in dry dichloromethane is added. The reaction is refluxed for 8 hours. After cooling, the solvent is removed under reduced pressure. A solution of KOH in ethylene glycol (424 mmol) and hydrazine monohydrate (80 mmol) is added to the residue before heating to reflux for 4 hours. After cooling, the reaction is subjected to standard work up, and purified using standard protocols to afford 6-bromo-2,3-dihydro-1H-benzo[de]isoquinoline 6. (Similarly described in Journal of the American Chemical Society (2003), 125(19), 5786-5791).

Step 4: To a solution of 6-bromo-2,3-dihydro-1H-benzo[de]isoquinoline 6 (1 eq) in THE (10 vol eq) at 0° C. is added NaH (5 eq) and stirred at this temp for 15 min before the addition of 3-bromopiperidine-2,6-dione 7 (1 eq). The reaction mixture is slowly heated to 60° C. and stirred at this temperature until completion of the reaction. A standard workup and purification using standard protocols affords 3-(6-bromo-1H-benzo[de]isoquinolin-2(3H)-yl)piperidine-2,6-dione Compound 28.

### Example 27. Synthesis of 3-(6-bromo-1H-perimidin-1-yl)piperidine-2,6-dione (Compound 29) and 3-(7-bromo-1H-perimidin-1-yl)piperidine-2,6-dione (Compound 30)

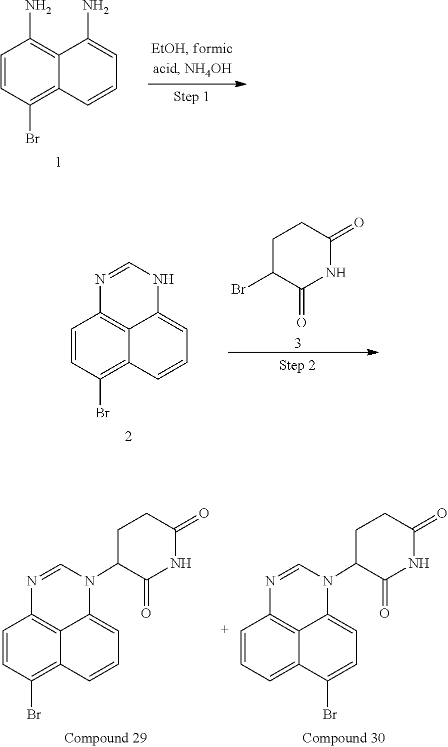

Step 1: Dry 4-bromonaphthalene-1,8-diamine 1 (17.1 mmol) is crushed with a mortar and pestle and dissolved in 12 mL of absolute ethanol. Formic acid (106 mmol) is added and the reaction is allowed to stir at reflux for 40 minutes. The reaction is diluted with water (2 mL) and basified with 2N NH₄OH The resulting precipitate is filtered, washed with ether and recrystallized in ethanol to afford 6-bromo-1H-perimidine 2.

Step 2: In an oven-dried flask, 6-bromo-1H-perimidine 2 (385.65 μmol) is dissolved in THF (10 mL) and then cooled to 0° C. Sodium hydride (60% dispersion in mineral oil, 147.77 mg, 3.86 mmol) is added portionwise and stirred at 0° C. for 30 mins. 3-bromopiperidine-2,6-dione 3 (1.93 mmol) is added and reaction mixture is stirred at RT for 30 mins then 0° C. for 16 hours. The progress of the reaction is monitored by TLC and after reaction completion the reaction mixture is quenched with chilled water, extracted with ethyl acetate, and organic layers washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-1H-perimidin-1-yl)piperidine-2,6-dione Compound 29 and 3-(7-bromo-1H-perimidin-1-yl)piperidine-2,6-dione Compound 30.

### Example 28. Synthesis of 3-(7-bromo-1-benzo[de]cinnolin-1-yl)piperidine-2,6-dione (Compound 31)

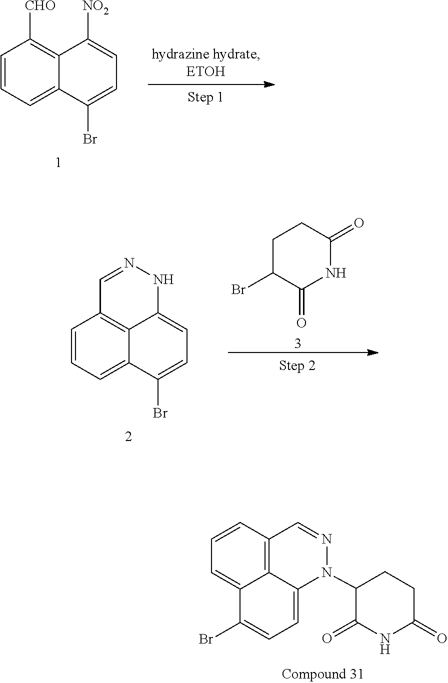

Step 1: A mixture of 1 mmol of 5-bromo-8-nitro-1-naphthaldehyde 1 and 1 ml of 88% hydrazine hydrate in 10 ml of ethanol is heated for 6 h at reflux in an argon atmosphere. The mixture is then cooled and poured into 20 ml of water and the precipitate is filtered off and dried to afford 7-bromo-1H-benzo[de]cinnoline 2

Step 2: A solution of 7-bromo-11H-benzo[de]cinnoline 2 (385.65 μmol) dissolved in THF (10 mL) is cooled to 0° C. then sodium hydride (60% dispersion in mineral oil, 147.77 mg, 3.86 mmol) is added portionwise and stirred at 0° C. for 30 mins. 3-bromopiperidine-2,6-dione (1.93 mmol) is added and reaction mixture is stirred at RT for 30 mins and then stirred at 0° C. for 16 hours. The progress of the reaction is monitored by TLC and after reaction completion, the reaction mixture is quenched with chilled water, extracted with ethyl acetate, and washed with brine. The organic layer is separated, dried over anhydrous sodium sulfate, filtered, and concentrated under reduced pressure to afford the crude compound. The crude material is purified by column chromatography by eluting with 10 to 50% ethyl acetate to afford 3-(7-bromo-1H-benzo[de]cinnolin-1-yl)piperidine-2,6-dione Compound 31.

### Example 29. Synthesis of 3-(6-bromo-1H-naphtho[1,8-de][1,2,3]triazin-1-yl)piperidine-2,6-dione (Compound 32) and 3-(7-bromo-1H-naphtho[1,8-de][1,2,3]triazin-1-yl)piperidine-2,6-dione (Compound 33)

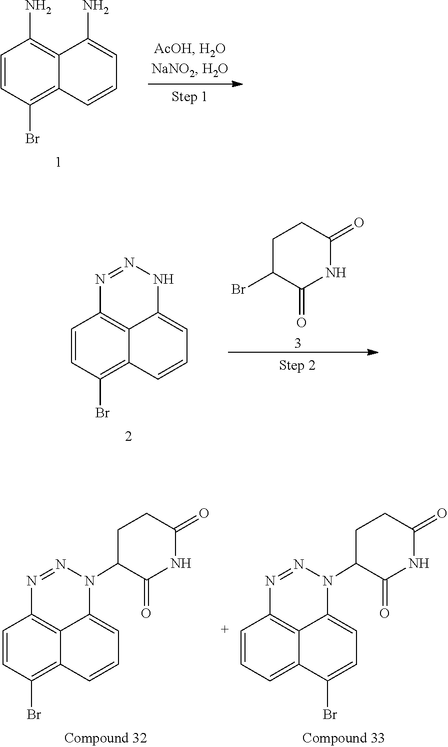

Step 1: A solution of 4-bromonaphthalen-1,8-diamine 1 (0.014 mol) is suspended in H₂O (600 mL) and AcOH (20 mL) and refluxed. The hot suspension is filtered (filter crucible with celite) and cooled to RT. NaNO₂ (1.55 g, 0.032 mol) in H₂O (20 mL) is added dropwise. The reaction mixture is stirred for 5 hr then filtered (filter crucible), washed with hot H₂O, and dried overnight to afford 6-bromo-1H-naphtho[1,8-de][1,2,3]triazine 2.

Step 2: A solution of 6-bromo-1H-naphtho[1,8-de][1,2,3]triazine 2 (385.65 μmol) dissolved in THE (10 mL) is cooled to 0° C. then sodium hydride (60% dispersion in mineral oil, 147.77 mg, 3.86 mmol) is added portionwise and stirred at 0° C. for 30 mins. 3-bromopiperidine-2,6-dione 3 (1.93 mmol) is added and reaction mixture is stirred at RT for 30 mins and then stirred at 0° C. for 16 hours. The progress of the reaction is monitored by TLC and after reaction completion reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-1H-naphtho[1,8-de][1,2,3]triazin-1-yl)piperidine-2,6-dione Compound 32 and 3-(7-bromo-1H-naphtho[1,8-de][1,2,3]triazin-1-yl)piperidine-2,6-dione Compound 33.

### Example 30. Synthesis of 3-(6-bromo-2H-naphtho[1,8-cd]isoxazol-2-yl)piperidine-2,6-dione (Compound 34)

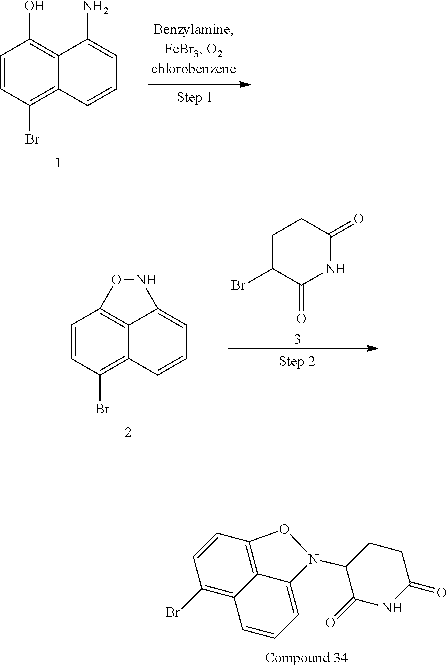

Step 1: A solution of 8-amino-4-bromonapthalenol 1 (1.0 mmol), benzylamine (1.3 mmol), FeBr₃ and dry chlorobenzene (1 mL) is added to an oven-dried Schlenk tube. The tube is equipped with a molecular oxygen balloon. The reaction mixture is stirred at 110° C. The reaction is monitored for complete consumption of starting material by TLC. The reaction is cooled to RT, diluted with CH₂Cl₂, and washed with water. The organic layer is dried over anhydrous Na₂SO₄, filtered, and concentrated under reduced pressure. The crude material is purified by silica column chromatography (ethyl acetate/hexane) to afford 6-bromo-2H-naptho[1,8-cd]isoxazole 2.

Step 2: To a stirred solution of 3-bromopiperidine-2,6-dione 3 (1.0 mmol) and DIPEA (2.5 mmol) in DMF (3 mL) is added 6-bromo-2H-naptho[1,8-cd]isoxazole 2 (2.5 mmol). The resulting solution is heated at 80° C.-100° C. for 5 hr. The reaction mixture is then cooled to room temperature and evaporated under reduced pressure. The crude reaction ix is purified by reverse phase preparative HPLC to afford 3-(6-bromo-2H-naphtho[1,8-cd]isoxazol-2-yl)piperidine-2,6-dione Compound 34.

### Example 31. Synthesis of 3-(6-bromo-2-oxo-2,3-dihydro-1H-perimidin-1-yl)piperidine-2,6-dione (Compound 35) and 3-(7-bromo-2-oxo-2,3-dihydro-1H-perimidin-1-yl)piperidine-2,6-dione (Compound 36)

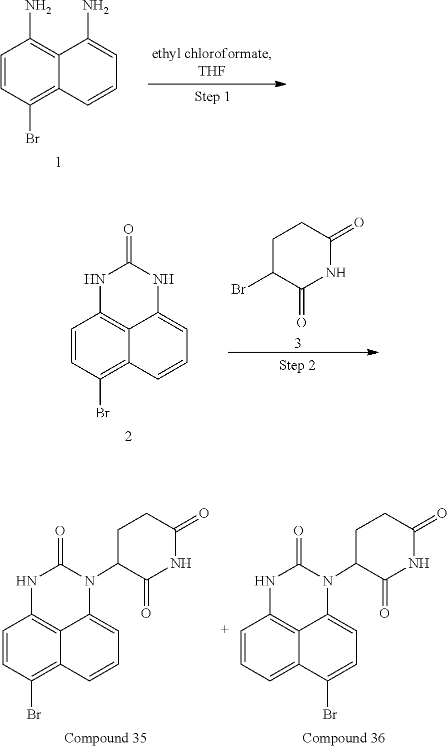

Step 1: To a solution of 4-bromonaphthalene-1,8-diamine 1 (31.6 mmol) in 100 mL THF is added dropwise a solution of ethyl chloroformate (31.6 mmol) in 10 mL THF over a period of 30 min at 0° C. The mixture is stirred at 25° C. for 1 d and then heated at 40° C. for 2 h. The precipitate is filtered and washed with CH₂Cl₂ to afford 6-bromo-1H-perimidin-2(3H)-one.

Step 2: 6-bromo-1H-perimidin-2(3H)-one 2 (385.65 μmol) is dissolved in THF (10 mL) and then cooled to 0° C. Sodium hydride (60% dispersion in mineral oil, 147.77 mg, 3.86 mmol) is added portionwise and stirred at 0° C. for 30 min. 3-bromopiperidine-2,6-dione 3 (1.93 mmol) is added and the reaction mixture is stirred at RT for 30 mins and then stirred at 0° C. for 16 hours. The progress of the reaction is monitored by TLC and after reaction completion the reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-2-oxo-2,3-dihydro-1H-perimidin-1-yl)piperidine-2,6-dione Compound 35 and 3-(7-bromo-2-oxo-2,3-dihydro-1H-perimidin-1-yl)piperidine-2,6-dione Compound 36.

### Example 32. Synthesis of 3-(6-bromo-2-oxo-2,3-dihydro-1H-benzo[de]quinolin-1-yl)piperidine-2,6-dione (Compound 37)

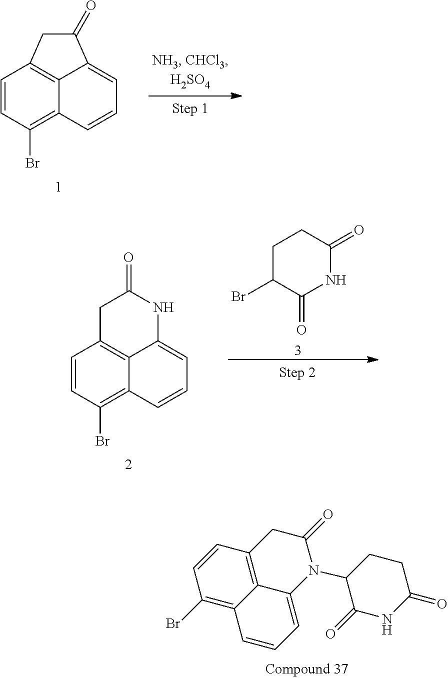

Step 1: A mixture of 5-broroacenaphthylen-1(2H)-one 1 (3 g) and 0.8N NH₃ in 80 ml CHCl₃, is stirred with 2 nit concentrated H₂SO₄ at 50° C. for 0.5 hour and then cooled to 0° C. The mixture is neutralized with saturated aq. KHCO₃ and filtered. The organic layer of the filtrate is washed with water and brine, dried over anhydrous sodium sulfate, filtered, and concentrated under reduced pressure. The resulting residue is purified by silica gel chromatography (ethyl acetate/hexanes) to afford 6-bromo-1H-benzo[de]quinolin-2(3H)-one 2.

Step 2: 6-bromol-1-benzo[de]quinolin-2(3H)-one 2 (385.65 μmol) is dissolved in THF (10 mL) and then cooled to 0° C. Sodium hydride (60% dispersion in mineral oil, 147.77 mg, 3.86 mmol) is added portionwise and stirred at 0° C. for 30 mins. 3-bromopiperidine-2,6-dione (1.93 mmol) is added and reaction mixture is stirred at RT for 30 mins and then stirred at 0° C. for 16 hr. The progress of the reaction is monitored by TLC and after reaction completion the reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-2-oxo-2,3-dihydro-1H-benzo[de]quinolin-1-yl)piperidine-2,6-dione Compound 37.

### Example 33. Synthesis of 3-(6-bromo-2-oxonaphtho[1,8-de][1,3]oxazin-3(2H)-yl)piperidine-2,6-dione (Compound 38) and 3-(7-bromo-2-oxonaphtho[1,8-de][1,3]oxazin-3(2H)-yl)piperidine-2,6˜dione (Compound 39)

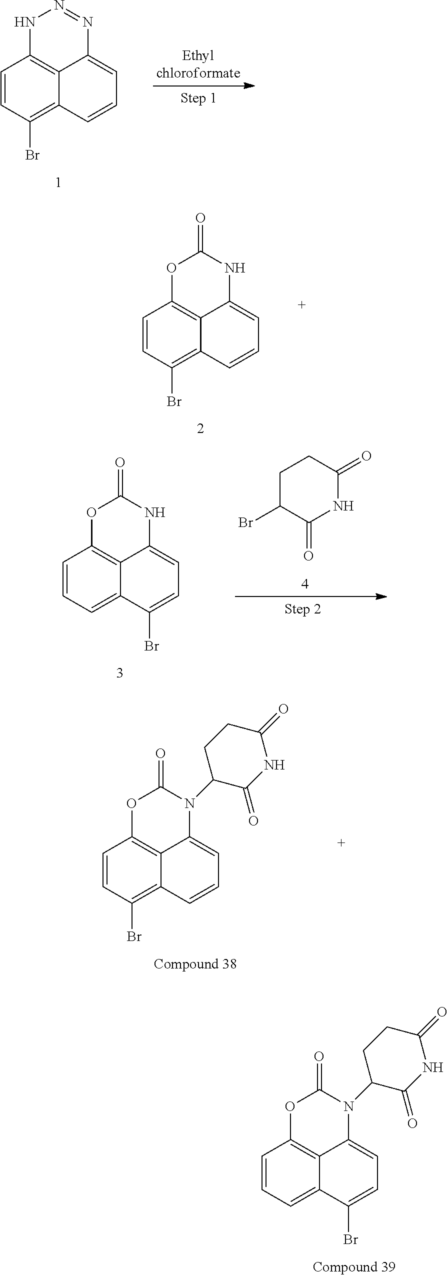 [SMILES: Brc1ccc2c3c(cccc13)N=NN2 ; O=C1Nc2cccc3c(Br)ccc(c23)O1 ; O=C1Nc2ccc(Br)c3cccc(c23)O1 ; O=C1CCC(Br)C(=O)N1 ; O=C1CCC(N2C(=O)Oc3ccc(Br)c4cccc2c34)C(=O)N1 ; O=C1CCC(N2C(=O)Oc3cccc4c(Br)ccc2c34)C(=O)N1]

Step 1: A microwave tube is charged with 6-bromo-1H-naphtho[1,8-de][1,2,3]triazine 1 and ethyl chloroformate, sealed and heated to 200° C. for 4 minutes. The reaction is cooled and concentrated. The crude residue is purified by silica gel chromatography to afford a mixture of 7-bromonaphtho[1,8-de][1,3]oxazin-2(3H)-one 2 and 6-bromonaphtho[1,8-de][1,3]oxazin-2(3H)-one 3.

Step 2: A mixture of 7-bromonaphtho[1,8-de][1,3]oxazin-2(3H-one 2 and 6-bromonaphtho[1,8-de][1,3]oxazin-2(3H)-one 3 (385.65 μmol) is dissolved in THF (10 mL) and then cooled to 0° C. Sodium hydride (60% dispersion in mineral oil, 147.77 mg, 3.86 mmol) is added portionwise and stirred at 0° C. for 30 mins. 3-bromopiperidine-2,6-dione (1.93 mmol) is added and reaction mixture is stirred at RT for 30 mins and then stirred at 0° C. for 16 hr. The progress of the reaction is monitored by TLC and after reaction completion the reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(7-bromo-2-oxonaphtho[1, 8-de][1,3]oxazin-3(2H)-yl)piperidine-2,6-dione Compound 38 and 3-(6-bromo-2-oxonaphtho[1,8-de][1,3]oxazin-32H)-yl)piperidine-2,6-dione Compound 39.

### Example 34. Synthesis of 3-(6-bromo-1,1-dioxido-2H-naphtho[1,8-cd]isothiazol-2-yl)piperidine-2,6-dione (Compound 40)

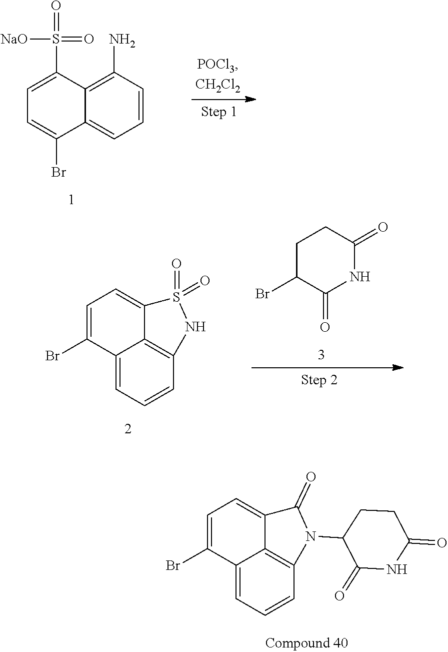

Step 1: Sodium 4-bromo-8-amino-naphthalene-1-sulfonate 1 (1.2 g, 3.70 mmol) is suspended in phosphorous oxychloride (10 mL, 107 5 mmol) and the mixture is refluxed for 1 hour to give a thin suspension. The mixture is cooled to room temperature and is added to ice (100 mL). The precipitate is collected and washed with water (20 mL) then dried under vacuum. The solid is dissolved in 5% methanol in methylene chloride and placed on a silica gel column and eluted with 5% methanol in methylene chloride to afford 6-bromo-2H-naphtho[1,8-cd]isothiazole 1,1-dioxide 2.

Step 2: 6-bromo-2H-naphtho[1,8-cd]isothiazole 1,1-dioxide 2 (385.65 μmol) is dissolved in THE (10 mL) and then cooled to 0° C. Sodium hydride (60% dispersion in mineral oil, 147.77 mg, 3.86 mmol) is added portionwise and stirred at 0° C. for 30 mins. 3-bromopiperidine-2,6-dione (1.93 mmol) is added and reaction mixture is stirred at RT for 30 mins and then stirred at 0° C. for 16 hr. The progress of the reaction is monitored by TLC and after reaction completion the reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 3-(6-bromo-2-oxo-2,3-dihydro-1H-benzo[de]quinolin-1-yl)piperidine-2,6-dione Compound 40.

### Example 35. Synthesis of 5-(6-bromo-2-oxobenizo[ed]indol-1(2H)-yl)-1,3-oxazinane-2,4-dione (Compound 41)

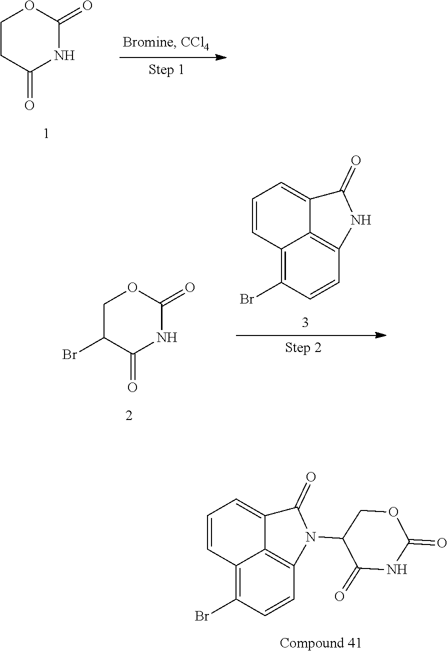 [SMILES: O=C1CCOC(=O)N1 ; O=C1NC(=O)C(Br)CO1 ; O=C1Nc2ccc(Br)c3cccc1c23 ; O=C1NC(=O)C(N2C(=O)c3cccc4c(Br)ccc2c34)CO1]

Step 1: A solution of bromine (87.8 mmol) is added to a suspension of 1,3-oxazinane-2,4-dione 1 (50.3 mmol) suspended in chloroform (20 ml) and the mixture is stirred in a closed vessel for 90 minutes at a temperature of 110° C. After cooling, the vessel is opened and stirring is continued until no more hydrogen bromide escapes. The reaction mixture is concentrated under reduced pressure. The residue is dissolved in ethanol and evaporated to afford 5-bromo-1,3-oxazinane-2,4-dione.

Step 2: 6-bromobenzo[cd]indol-2(1H)-one 3 (385.65 μmol) is dissolved in THF (10 mL) and then cooled to 0° C. Sodium hydride (60% dispersion in mineral oil, 147.77 mg, 3.86 mmol) is added portionwise and stirred at 0° C. for 30 mins. 5-bromo-1,3-oxazinane-2,4-dione 2 (1.93 mmol) is added and reaction mixture is stirred at RT for 30 mins and then stirred at 0° C. for 16 hr. The progress of the reaction is monitored by TLC and after reaction completion the reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 5-(6-bromo-2-oxobenzo[cd]indol-1(2H)-yl)-1,3-oxazinane-2,4-dione Compound 41.

### Example 36. Synthesis of 5-(6-bromo-2-oxobenzo[cd]indol-1(2H)-yl)pyrimidine-2,4(3H,5H)-dione (Compound 42)

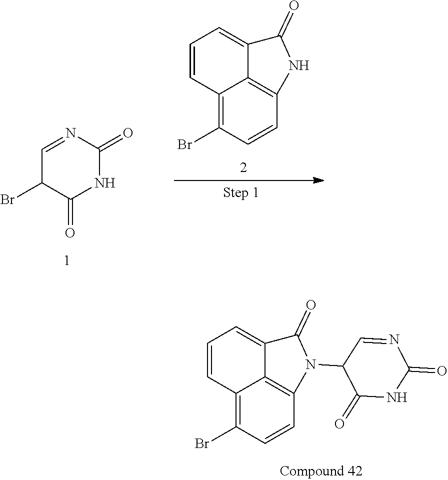 [SMILES: O=C1N=CC(Br)C(=O)N1 ; O=C1Nc2ccc(Br)c3cccc1c23 ; O=C1N=CC(N2C(=O)c3cccc4c(Br)ccc2c34)C(=O)N1]

Step 1: To a solution of 6-bromobenzo[cd]indol-2(1H)-one 2 (385.65 μmol) is dissolved in THE (10 mL) and then cooled to 0° C. Sodium hydride (60% dispersion in mineral oil, 147.77 mg, 3.86 mmol) is added portionwise and stirred at 0° C. for 30 mins. 5-bromopyrimidine-2,4(3H,5H)-dione 1 (as prepared in PCT Int. Appl., 2016044770, 1.93 mmol) is added and reaction mixture is stirred at RT for 30 mins and then stirred at 0° C. for 16 hr. The progress of the reaction is monitored by TLC and after reaction completion the reaction mixture is quenched with chilled water, extracted with ethyl acetate, and washed with brine. The organic layer is separated and dried over anhydrous sodium sulfate, filtered and concentrated under reduced pressure to afford the crude compound. The crude material is purified by column chromatography by eluting with 10 to 50% ethyl acetate to afford 5-(6-bromo-2-oxobenzo[cd]indol-1(2H)-yl)pyrimidine-2,4(3H,5H)-dione Compound 42.

### Example 37. Synthesis of 3-(5-bromo-2-oxo-benzo[cd]indol-1-yl)piperidine-2,6-dione (Compound 43)

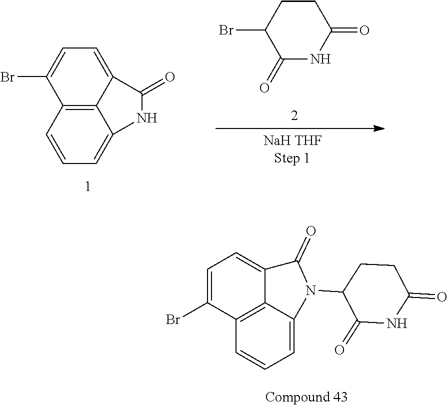 [SMILES: O=C1Nc2cccc3c(Br)ccc1c23 ; O=C1CCC(Br)C(=O)N1 ; O=C1CCC(N2C(=O)c3ccc(Br)c4cccc2c34)C(=O)N1]

Step 1: To a 500 mL three-necked round bottom flask containing a well stirred solution of 5-bromo-1H-benzo[cd]indol-2-one 1 (2.0 g, 6.85 mmol) in dry THE (200 mL) was added sodium hydride (60% dispersion in mineral oil, 2.63 g, 68.53 mmol) at 0° C. and the reaction mixture was stirred at ambient temperature. After 1 h, 3-bromopiperidine-2,6-dione 2 (6.58 g, 30.84 mmol) dissolved in dry THE (30 mL), was added at 0° C. The reaction mixture was stirred at 65° C. for 16 hours. The reaction mixture was quenched with saturated ammonium chloride solution (50 mL) at 0° C. then extracted with ethyl acetate (2×50 mL). The combined organic layers were combined, dried over anhydrous sodium sulfate, and concentrated under reduced pressure to get a crude compound which was purified by silica gel column chromatography and compound was eluted at 80-100% ethyl acetate in petroleum ether to afford 3-(5-bromo-2-oxo-benzo[cd]indol-1-yl)piperidine-2,6-di one Compound 43 (1.3 g, 2.85 mmol, 41.5700 yield, 78.700 purity) as a yellow solid. LCMS (ES+): m/z 359.0 [M+H]+

### Example 38. The Following Compounds can be Synthesized in a Similar Manner

| Intermediate | Product | Compound No. |
|---|---|---|
| 3-bromo-2-piperidinone CAS# 34433-86-8 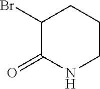 | 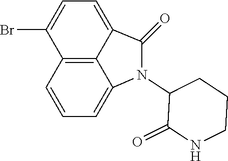 [SMILES: O=C1NCCCC1N1C(=O)c2ccc(Br)c3cccc1c23] | Compound 44 |
| 6-oxopiperidin-3-yl benzenesulfonate WO2011075699 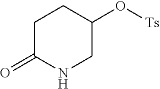 [SMILES: Cc1ccc(S(=O)(=O)OC2CCC(=O)NC2)cc1] | 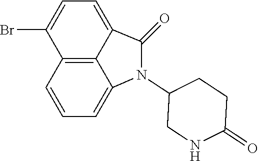 [SMILES: O=C1CCC(N2C(=O)c3ccc(Br)c4cccc2c34)CN1] | Compound 45 |
| 5-bromodihydro-2,4(1H,3H)- pyrimidinedione CAS# 1193-76-6 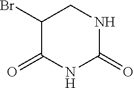 | 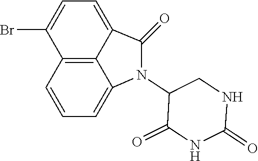 [SMILES: O=C1NCC(N2C(=O)c3ccc(Br)c4cccc2c34)C(=O)N1] | Compound 46 |
| WO2017197051A 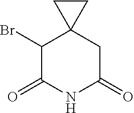 [SMILES: O=C1CC2(CC2)C(Br)C(=O)N1] | 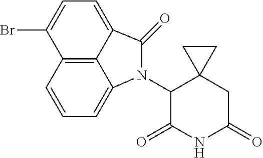 [SMILES: O=C1CC2(CC2)C(N2C(=O)c3ccc(Br)c4cccc2c34)C(=O)N1] | Compound 47 |
| 5-bromopyrimidine- 2,4(3H,5H)-dione (as prepared in PCT Int. Appl., 2016044770, 1.93 mmol) 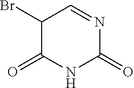 | 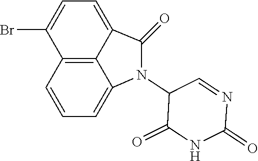 [SMILES: O=C1N=CC(N2C(=O)c3ccc(Br)c4cccc2c34)C(=O)N1] | Compound 48 |

### Example 39. The Following Amines Intermediates can be Converted to Bromo Intermediates Using Standard Chemistry and Utilized in the Preceding Alkylation Reaction to Prepares the Products

| Amine Starting | | | Compound |
|---|---|---|---|
| Material | Bromo intermediate | Product | No. |
| WO2017197051A 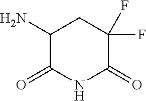 [SMILES: NC1CC(F)(F)C(=O)NC1=O] | 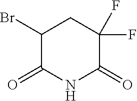 [SMILES: O=C1NC(=O)C(F)(F)CC1Br] | 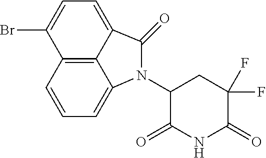 [SMILES: O=C1NC(=O)C(F)(F)CC1N1C(=O)c2ccc(Br)c3cccc1c23] | Compound 49 |
| WO2017197051A 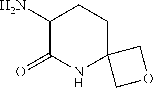 [SMILES: NC1CCC2(COC2)NC1=O] | 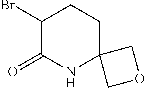 [SMILES: O=C1NC2(CCC1Br)COC2] | 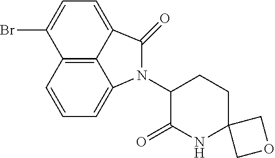 [SMILES: O=C1NC2(CCC1N1C(=O)c3ccc(Br)c4cccc1c34)COC2] | Compound 50 |
| WO2017197051A 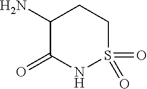 | 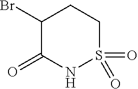 | 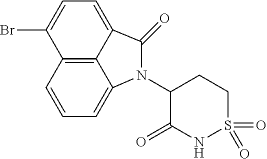 [SMILES: O=C1NS(=O)(=O)CCC1N1C(=O)c2ccc(Br)c3cccc1c23] | Compound 51 |
| WO2017197051A 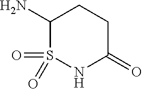 | 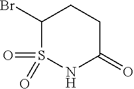 | 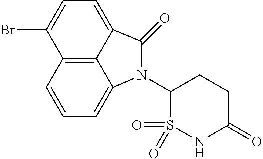 [SMILES: O=C1CCC(N2C(=O)c3ccc(Br)c4cccc2c34)S(=O)(=O)N1] | Compound 52 |
| WO2017197051A 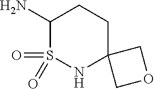 [SMILES: NC1CCC2(COC2)NS1(=O)=O] | 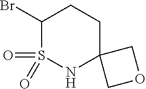 [SMILES: O=S1(=O)NC2(CCC1Br)COC2] | 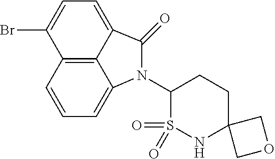 [SMILES: O=C1c2ccc(Br)c3cccc(c23)N1C1CCC2(COC2)NS1(=O)=O] | Compound 53 |
| WO2017197051A 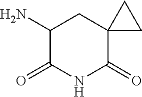 [SMILES: NC1CC2(CC2)C(=O)NC1=O] | 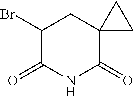 [SMILES: O=C1NC(=O)C2(CC2)CC1Br] | 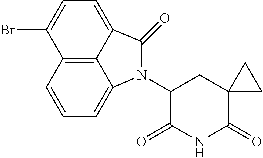 [SMILES: O=C1NC(=O)C2(CC2)CC1N1C(=O)c2ccc(Br)c3cccc1c23] | Compound 54 |

### Example 40. Synthesis of 3-(5-bromo-2-oxobenzo[cd]indol-1(2H)-yl)azepane-2,7-dione (Compound 55)

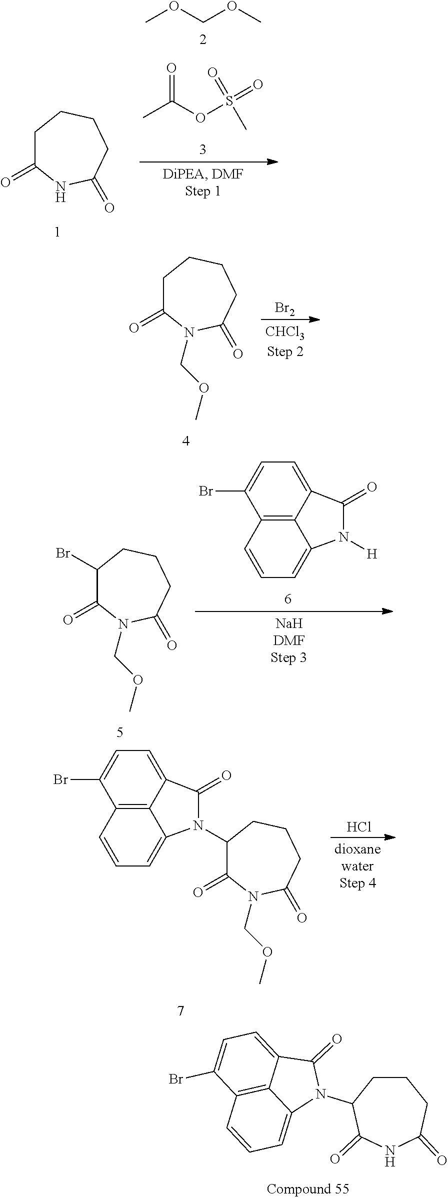

Step 1: Dimethoxymethane 2 (4 eq) is added at 0° C. to acetylmethanesulfonate 3 (4 eq) and the reaction stirred at 25° C. for 2 hours. A solution of 2,7-azepanedione 1 (1 eq, CAS #4726-93-6) and DiPEA (4 eq) in DMF is added to the reaction mixture over 45 min, then stirred for 15 min. Standard work up and purification using standard protocols affords 1-(methoxymethyl)azepane-2,7-dione 4. (As described in US2003375340)

Step 2: A solution of 1-(methoxymethyl)azepane-2,7-dione 4 (1 eq) and Br₂ (1 eq) in CHCl₃ in a sealed tube is heated at 110° C. for 1.5 hours. Standard workup and purification using standard protocols affords 3-bromo-1-(methoxymethyl)azepane-2,7-dione 5.

Step 3: To a solution of 5-bromobenzo[cd]indol-2(1H)-one 6 (5 eq) in THE at 0° C. is added NaH (60% in oil, 10 eq), in portions, at 0° C. and the reaction mixture is stirred at room temperature for 60 min. The reaction mixture is cooled to 0° C., 3-bromo-1-(methoxymethyl)azepane-2,7-dione (1 eq) in THE is added slowly, the cooling bath removed, and the reaction mixture is slowly heated to 65° C. The reaction mixture is stirred at this temperature until the reaction is judged complete. A standard workup and purification using standard protocols affords 3-(5-bromo-2-oxobenzo[cd]indol-1(2H)-yl)-1-(methoxymethyl)azepane-2,7-dione 7.

Step 4: To a solution of 3-(5-bromo-2-oxobenzo[cd]indol-1(2H)-yl)-1-(methoxymethyl)azepane-2,7-dione 7 (1 eq) in dioxane, water and concentrated HCl is heated at reflux until the reaction is judged complete. A standard workup and purification using standard protocols affords 3-(5-bromo-2-oxobenzo[cd]indol-1(2H)-yl)azepane-2,7-dione Compound 55.

### Example 41. Synthesis of 3-(5-bromo-2-oxobenzo[cd]indol-1(2H)-yl)pyrrolidine-2,5-dione (Compound 56)

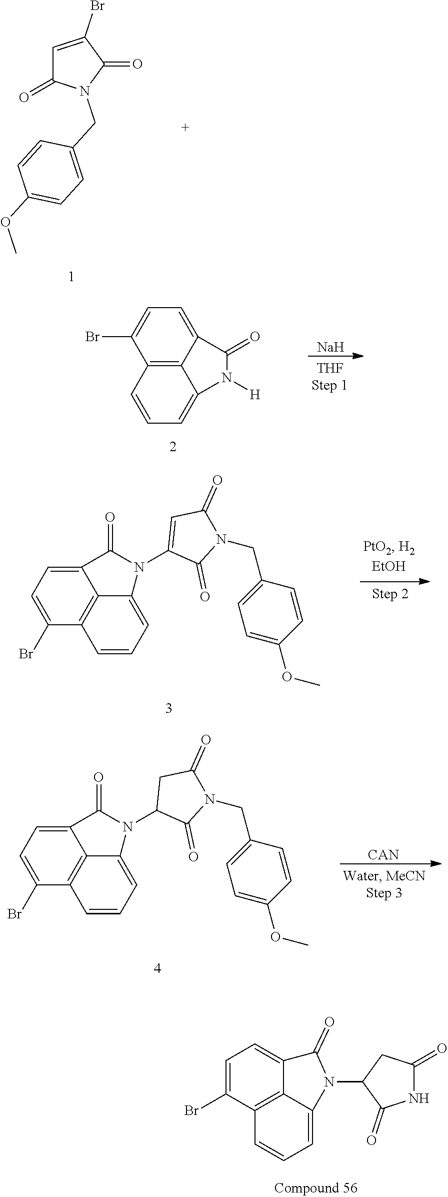

Step 1: To a solution of 3-bromo-1-(methoxymethyl)azepane-2,7-dione 2 (5 eq) in THE at 0° C. is added NaH (60% in oil, 10 eq), in portions, at 0° C. and the reaction mixture is stirred at room temperature for 60 min. The reaction mixture is cooled to 0° C., 3-bromo-1-{[4-(methyloxy)phenyl]methyl}-1H-pyrrole-2,5-dione 1 (WO2008074716, 1 eq) in THE is added slowly, and the cooling bath removed. The reaction mixture is slowly heated to 65° C. and the reaction mixture is stirred at this temperature until the reaction is judged complete. A standard workup and purification affords 3-(5-bromo-2-oxobenzo[cd]indol-1(2H)-yl)-1-(4-methoxybenzyl)-1H-pyrrole-2,5-dione 3.

Step 2: A suspension of 3-(5-bromo-2-oxobenzo[cd]indol-1(2H)-yl)-1-(4-methoxybenzyl)-1H-pyrrole-2,5-dione 3 and catalytic PtO₂ in in EtOH is stirred under an atmosphere of hydrogen, at appropriate pressure and temperature, to afford 3-(5-bromo-2-oxobenzo[cd]indol-1(2H)-yl)-1-(4-methoxybenzyl)pyrrolidine-2,5-dione 4 after standard workup protocols.

Step 3: To a solution of 3-(5-bromo-2-oxobenzo[cd]indol-1(2H)-yl)-1-(4-methoxybenzyl)pyrrolidine-2,5-dione 4 in acetonitrile and water, is added CAN (1-3 eq). The resulting mixture is stirred at room temperature until the reaction is judged complete. Standard workup and purification using standard protocols affords 3-(5-bromo-2-oxobenzo[cd]indol-1(2H)-yl)pyrrolidine-2,5-dione Compound 56.

### Example 42. Synthesis of 5-(6-bromo-2-oxobenzo[cd]indol-1(2H)-yl)-1,3-diazabicyclo[3.1.1]heptane-2,4-dione (Compound 57)

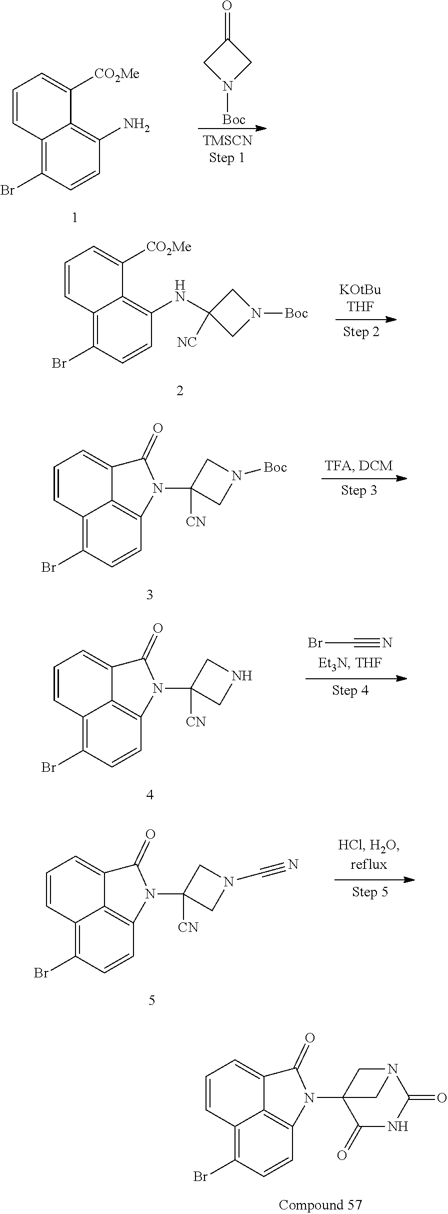

Step 1: To a stirred solution of tert-butyl 3-oxoazetidine-1-carboxylate (1 eq) in methanol (10 vol eq) is added methyl 8-amino-5-bromo-1-naphthoate 1 (1.1) followed by TMSCN (2 eq) and the reaction mixture is stirred for 16 hours at room temperature. A standard workup and purification provides tert-butyl 3-((4-bromo-8-(methoxycarbonyl)naphthalen-1-yl)amino)-3-cyanoazetidine-1-carboxylate 2.

Step 2: To a stirred solution of tert-butyl 3-((4-bromo-8-(methoxycarbonyl)naphthalen-1-yl)amino)-3-cyanoazetidine-1-carboxylate 2 (1 eq) in THE (10 vol eq) is added potassium tert-butoxide (2 eq) at 0° C. and the reaction mixture allowed to warm to room temperature. The reaction mixture is neutralized with 1M citric acid solution (to adjust to pH 6) and diluted with ethyl acetate. A standard workup and purification will afford tert-butyl 3-(6-bromo-2-oxobenzo[cd]indol-1(2H)-yl)-3-cyanoazetidine-1-carboxylate 3.

Step 3: To a stirred mixture of tert-butyl 3-(6-bromo-2-oxobenzo[cd]indol-1(2H)-yl)-3-cyanoazetidine-1-carboxylate 3 (1 eq) in DCM (15 vol eq) is added trifluoroacetic acid (3 vol eq). The mixture is stirred at room temperature until completion and a standard workup and purification provides 3-(6-bromo-2-oxobenzo[ed]indol-1(21)-yl)azetidine-3-carbonitrile 4.

Step 4: To a solution of cyanogen bromide (1 eq) and sodium acetate (1 eq) in dry ethanol (20 vol eq) is added 3-(6-bromo-2-oxobenzo[cd]indo-1(2H)-yl)azetidine-3-carbonitrile 4 (1 eq) and the reaction mixture stirred at room temperature for 24 hours. A standard workup and purification will yield 3-(6-bromo-2-oxobenzo[cd]indol-1(2H)-yl)azetidine-1,3-dicarbonitrile 5.

Step 5: 3-(6-bromo-2-oxobenzo[cd]indol-1(2H)-yl)azetidine-1,3-dicarbonitrile 5 (1 eq mmol) is dissolved in 4.6 M aq. HCl and refluxed for 2 hours. After cooling to 0° C., a standard workup and purification will provide 5-(6-bromo-2-oxobenzo[cd]indol-1(2H)-yl)-1,3-diazabicyclo[3.1.1]heptane-2,4-dione Compound 57.

### Example 43. Synthesis of 1-(6-bromo-2-oxobenzo[cd]indol-1(2H)-yl)-3-azabicyclo[3.1.1]heptane-2,4-dione (Compound 58)

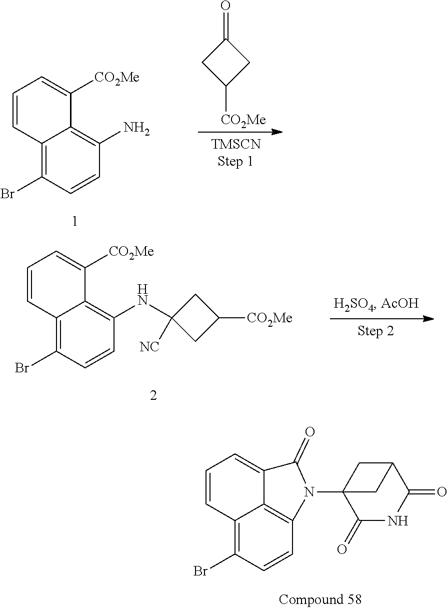 [SMILES: COC(=O)c1cccc2c(Br)ccc(N)c12 ; COC(=O)C1CC(=O)C1 ; COC(=O)c1cccc2c(Br)ccc(NC3(C#N)CC(C(=O)OC)C3)c12 ; O=C1NC(=O)C2(N3C(=O)c4cccc5c(Br)ccc3c45)CC1C2]

Step 1: To a stirred solution of methyl 3-oxocyclobutane-1-carboxylate (1 eq) in methanol (10 vol eq) is added methyl 8-amino-5-bromo-1-naphthoate 1 (1.1 eq) and the reaction is stirred at room temperature for one hour. The reaction mixture cooled to 0° C. and trimethylsilyl cyanide (2 eq) is added dropwise. The cooling bath is removed and the reaction mixture stirred at room temperature for 16 hours. A standard workup and purification affords methyl 5-bromo-8-((1-cyano-3-(methoxycarbonyl)cyclobutyl)amino)-1-naphthoate (2).

Step 2: To a stirred solution of methyl 5-bromo-8-((1-cyano-3-(ethoxycarbonyl)cyclobutyl)amino)-1-naphthoate (2) (1 eq) in acetic acid (30 eq) is added sulfuric acid (0.7 eq), and the reaction mixture is refluxed for 16 hours. A standard workup and purification affords 1-(6-bromo-2-oxobenzo[cd]indol-1(2H)-yl)-3-azabicyclo[3.1.1]heptane-2,4-dione Compound 58.

### Example 44. Synthesis of tert-butyl 4-[1-(2,6-dioxo-3-piperidyl)-2-oxo-benzo[cd]indol-6-yl]-3,6-dihydro-2H-pyridine-1-carboxylate (Compound 59)

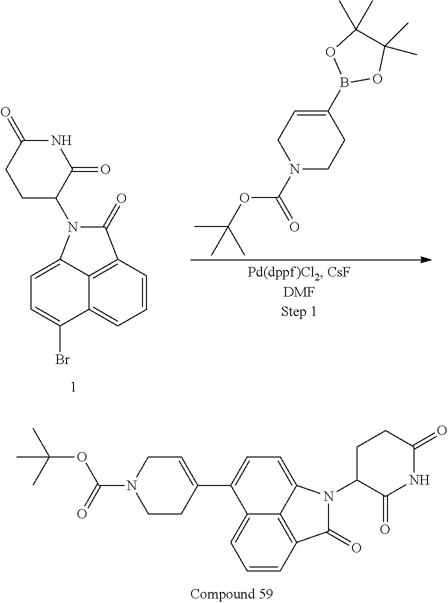 [SMILES: O=C1CCC(N2C(=O)c3cccc4c(Br)ccc2c34)C(=O)N1 ; CC(C)(C)OC(=O)N1CC=C(B2OC(C)(C)C(C)(C)O2)CC1 ; CC(C)(C)OC(=O)N1CC=C(c2ccc3c4c(cccc24)C(=O)N3C2CCC(=O)NC2=O)CC1]

Step 1: To a solution of 3-(6-bromo-2-oxo-benzo[cd]indol-1-yl)piperidine-2,6-dione 1 (740 mg, 2.06 mmol) in DMF (12 mL) was added tert-butyl 4-(4,4,5,5-tetramethyl-1,3,2-dioxaborolan-2-yl)-3,6-dihydro-2H-pyridine-1-carboxylate 2 (764.47 mg, 2.47 mmol) and cesium fluoride (469.44 mg, 3.09 mmol, 113.94 μL) at 25° C. and reaction mixture was degassed with nitrogen for 5 min. [1,1′-bis(diphenylphosphino)ferrocene]dichloropalladium(II) (168.25 mg, 206.03 μmol) was then added and again the reaction mixture was degassed with nitrogen for 5 min before the reaction mixture was stirred at 80° C. for 10h. Upon completion, the reaction mixture was cooled and poured into water (50 mL) and extracted with ethyl acetate (2×50 mL). Organic layers were combined, washed with brine (50 ml), and dried over anhydrous sodium sulfate then concentrated under reduced pressure. The crude material was purified by silica gel flash chromatography using 0-100% ethyl acetate in hexane as a eluent to give tert-butyl 4-[1-(2,6-dioxo-3-piperidyl)-2-oxo-benzo[cd]indol-6-yl]-3,6-dihydro-2H-pyridine-1-carboxylate Compound 59 (404 mg, 803.96 μmol, 39.02% yield, 91.84% purity) as a yellow solid. LCMS m/z [M+H]⁺=406.3 [M-56 (de tertiary butyl)] with purity 91.84%)

### Example 45. Synthesis of tert-butyl 4-[1-(2,6-dioxo-3-piperidyl)-2-oxo-benzo[cd]indol-6-yl]piperidine-1-carboxylate (Compound 60)

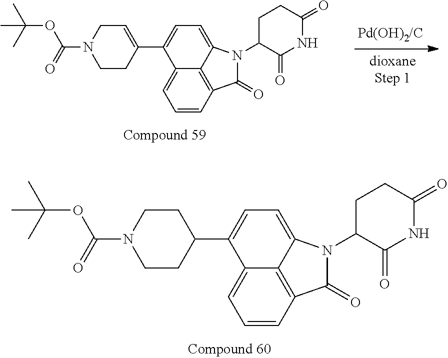 [SMILES: CC(C)(C)OC(=O)N1CC=C(c2ccc3c4c(cccc24)C(=O)N3C2CCC(=O)NC2=O)CC1 ; CC(C)(C)OC(=O)N1CCC(c2ccc3c4c(cccc24)C(=O)N3C2CCC(=O)NC2=O)CC1]

Step 1: To a solution of tert-butyl 4-[1-(2,6-dioxo-3-piperidyl)-2-oxo-benzo[cd]indol-6-yl]-3,6-dihydro-2H-pyridine-1-carboxylate Compound 59 (200 mg, 433.36 μmol) in 1,4-dioxane (10 mL) was added palladium hydroxide on carbon, 20 wt. % (100 mg, 712.08 μmol) under a nitrogen atmosphere and reaction mixture was stirred under a hydrogen atmosphere at 25° C. for 16 hours. Upon completion, the reaction mixture was filtered through a celite pad and the celite pad washed with ethyl acetate (100 mL) and the filtrate was concentrated under reduced pressure to give tert-butyl 4-[1-(2,6-dioxo-3-piperidyl)-2-oxo-benzo[cd]indol-6-yl]piperidine-1-carboxylate Compound 60 (201 mg, 380.60 μmol, 87.83% yield, 87.77% purity) as a yellow solid. LCMS m/z [M+H]⁺=364.2 ([M-100(De-tertiary butyl)].

### Example 46. Synthesis of 3-[2-oxo-6-(4-piperidyl)benzo[cd]indol-1-yl]piperidine-2,6-dione (Compound 61)

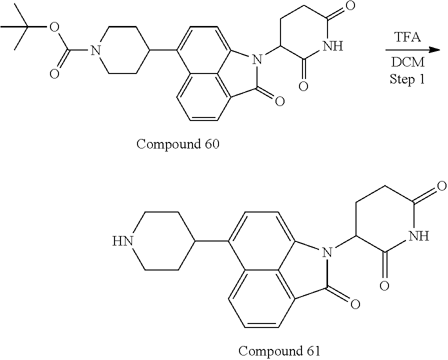 [SMILES: CC(C)(C)OC(=O)N1CCC(c2ccc3c4c(cccc24)C(=O)N3C2CCC(=O)NC2=O)CC1 ; O=C1CCC(N2C(=O)c3cccc4c(C5CCNCC5)ccc2c34)C(=O)N1]

Step 1: To a solution of tert-butyl 4-[1-(2,6-dioxo-3-piperidyl)-2-oxo-benzo[cd]indol-6-yl]piperidine-1-carboxylate Compound 60 (200 mg, 431.48 μmol) in DCM (4 mL) was added trifluoroacetic acid (983.97 mg, 8.63 mmol, 664.84 μL) at 0° C. and reaction mixture was allowed to stir at 25° C. for 4h. Upon completion, the reaction mixture was concentrated under reduced pressure and the residue triturated with methyl tert-butyl ether (2×20 mL) and the solvent was decanted to give a solid that was dried under reduced pressure to give 3-[2-oxo-6-(4-piperidyl)benzo[cd]indol-1-yl]piperidine-2,6-dione Compound 61 (192 mg, 213.94 μmol, 49.58% yield, 53.20% purity) as a light brown hygroscopic solid. LCMS m/z [M+H]⁺=364.3.

### Example 47. Synthesis of 9-[4-[1-(2,6-dioxo-3-piperidyl)-2-oxo-benzo[cd]indol-6-yl]-1-piperidyl]nonanoic acid (Compound 62)

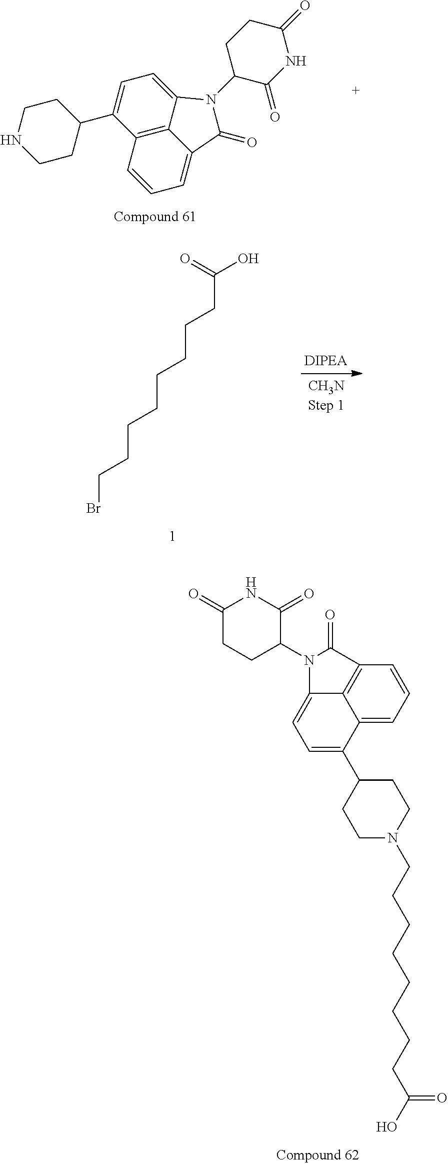 [SMILES: O=C1CCC(N2C(=O)c3cccc4c(C5CCNCC5)ccc2c34)C(=O)N1 ; O=C(O)CCCCCCCCBr ; O=C(O)CCCCCCCCN1CCC(c2ccc3c4c(cccc24)C(=O)N3C2CCC(=O)NC2=O)CC1]

Step 1: To a solution of 3-[2-oxo-6-(4-piperidyl)benzo[cd]indol-1-yl]piperidine-2,6-dione Compound 61 (50 mg, 104.73 μmol) in acetonitrile (2 mL) was added N-ethyl-N-isopropyl-propan-2-amine (94.75 mg, 733.09 μmol, 127.69 μL) and 9-bromononanoic acid 2 (24.83 mg, 104.73 μmol) at RT. Then the reaction was stirred for 4 h at 80° C. Progress of the reaction was monitored by LCMS. After completion of the reaction it was concentrated under reduced pressure, dissolved in EtOAc (200 mL), and washed with saturated NaHCO₃solution. The organic layer was dried over Na₂SO₄, filtered and concentrated. The crude material was triturated with diethyl ether and dried under reduced pressure to give 9-[4-[1-(2,6-dioxo-3-piperidyl)-2-oxo-benzo[cd]indol-6-yl]-1-piperidyl]nonanoic acid Compound 62 which was used in the next step without further purification. TLC: 10% MeOH in DCM (Rf: 0.2)

### Example 48. Synthesis of 3-[7-(difluoromethyl)-6-(1-methylpyrazol-4-yl)-3,4-dihydro-2H-quinolin-1-yl]-1-[1-[9-[4-[1-(2,6-dioxo-3-piperidyl)-2-oxo-benzo[cd]indol-6-yl]-1-piperidyl]nonanoyl]-4-piperidyl]-N-methyl-6,7-dihydro-4H-pyrazolo[4,3-c]pyridine-5-carboxamide formic acid salt (Compound 63)

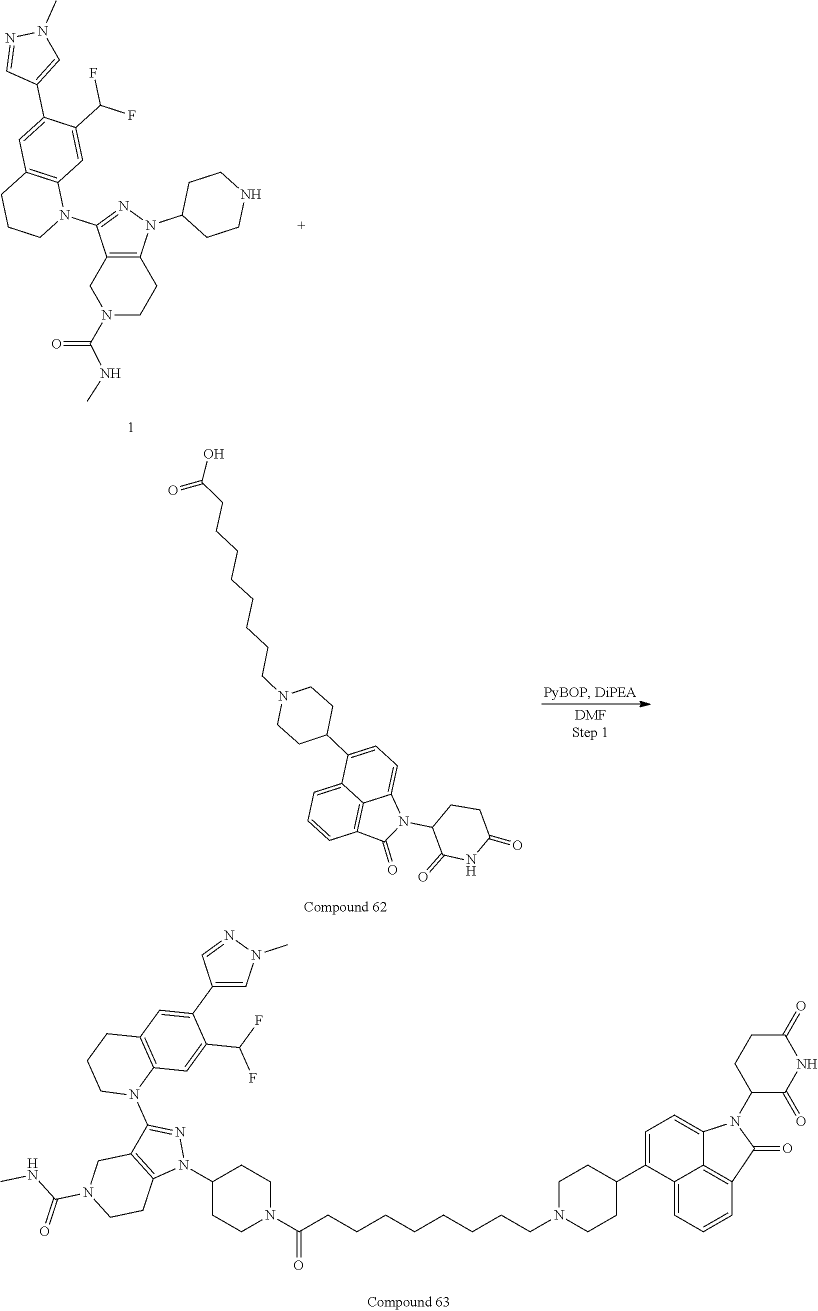 [SMILES: CNC(=O)N1CCc2c(c(N3CCCc4cc(-c5cnn(C)c5)c(C(F)F)cc43)nn2C2CCNCC2)C1 ; O=C(O)CCCCCCCCN1CCC(c2ccc3c4c(cccc24)C(=O)N3C2CCC(=O)NC2=O)CC1 ; CNC(=O)N1CCc2c(c(N3CCCc4cc(-c5cnn(C)c5)c(C(F)F)cc43)nn2C2CCN(C(=O)CCCCCCCCN3CCC(c4ccc5c6c(cccc46)C(=O)N5C4CCC(=O)NC4=O)CC3)CC2)C1]

Step 1: To a stirred solution of 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide 1 (47.15 mg, 78.91 μmol, 022) and 9-[4-[1-(2,6-dioxo-3-piperidyl)-2-oxo-benzo[cd]indol-6-yl]-1-piperidyl]nonanoic acid Compound 62 (50 mg, 78.91 μmol, 061) in DMF (2 mL) were added N-ethyl-N-isopropyl-propan-2-amine (81.59 mg, 631.26 μmol, 109.95 μL) and PyBOP (49.28 mg, 94.69 μmol) at RT. Then reaction mixture was stirred for 16 h at RT. Progress of the reaction was monitored by LCMS. After completion of the reaction, the reaction mixture was concentrated under reduced pressure to get crude product. The crude product was purified by reverse phase preparative HPLC to afford 3-[7-(difluoromethyl)-6-(1-methylpyrazol-4-yl)-3,4-dihydro-2H-quinolin-1-yl]-1-[1-[9-[4-[1-(2,6-dioxo-3-piperidyl)-2-oxo-benzo[cd]indol-6-yl]-1-piperidyl]nonanoyl]-4-piperidyl]-N-methyl-6,7-dihydro-4H-pyrazolo[4,3-c]pyridine-5-carboxamide formic acid salt Compound 63 (28.2 mg, 25.60 μmol, 32.44% yield, 97.32% purity) as pale yellow solid. LCMS m/z [M+H]⁺=1026.0. TLC: 10% MeOH in DCM (Rf: 0.1).

### Example 49. Synthesis of tert-butyl 4-[(2-oxo-1H-benzo[cd]indol-6-yl)amino]piperidine-1-carboxylate (Compound 64)

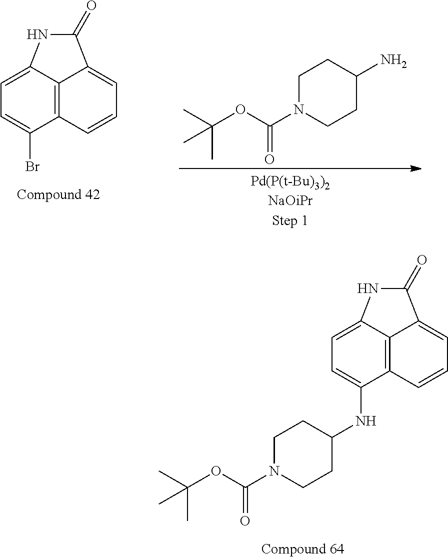 [SMILES: O=C1Nc2ccc(Br)c3cccc1c23 ; CC(C)(C)OC(=O)N1CCC(N)CC1 ; CC(C)(C)OC(=O)N1CCC(Nc2ccc3c4c(cccc24)C(=O)N3)CC1]

Step 1: To a stirred solution of tert-butyl 4-aminopiperidine-1-carboxylate (2.02 g, 10.08 mmol) and 6-bromo-1H-benzo[cd]indol-2-one Compound 42 (2.5 g, 10.08 mmol) in toluene (20 mL) was added sodium isopropoxide (2.91 g, 30.23 mmol) at RT. The reaction solution was degassed with nitrogen for 10 min and then Pd(P(t-Bu)₃)₂ (1.03 g, 2.02 mmol) was added to the reaction solution and again degassed with nitrogen for 5 min. Then the mixture was stirred for 16 h at 110° C. Progress of the reaction was monitored by LCMS. After completion of the reaction, it was filtered through celite and washed with EtOAc. The organic layer was washed with water and a brine solution. Organic layer was dried over anhydrous Na₂SO₄, filtered, and concentrated under reduced pressure to give the crude product. The crude product was purified by column chromatography using silica, 50% EA in Pet ether as eluent to afford tert-butyl 4-[(2-oxo-1H-benzo[cd]indol-6-yl)amino]piperidine-1-carboxylate Compound 64 (0.35, 914.43 μmol, 9.07% yield, 96% purity) as orange solid. TLC: 50% EA in Pet ether (Rf: 0.2)

### Example 50. Synthesis of tert-butyl 4-[[1-(2,6-dioxo-3-piperidyl)-2-oxo-benzo[cd]indol-6-yl]amino]piperidine-1-carboxylate (Compound 65)

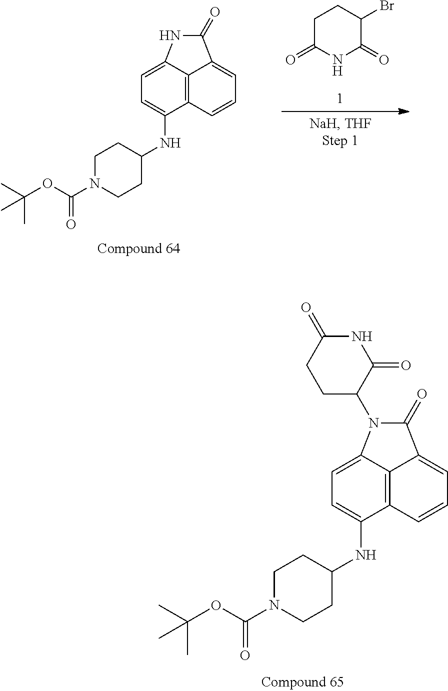 [SMILES: CC(C)(C)OC(=O)N1CCC(Nc2ccc3c4c(cccc24)C(=O)N3)CC1 ; O=C1CCC(Br)C(=O)N1 ; CC(C)(C)OC(=O)N1CCC(Nc2ccc3c4c(cccc24)C(=O)N3C2CCC(=O)NC2=O)CC1]

Step 1: To a stirred solution of tert-butyl 4-[(2-oxo-1H-benzo[cd]indol-6-yl)amino]piperidine-1-carboxylate Compound 64 (0.35 g, 952.54 μmol) in THE (4 mL) was added sodium hydride (342.88 mg, 14.29 mmol) at 0° C. and stirred for 1 h at RT. Then 3-bromopiperidine-2,6-dione (914.48 mg, 4.76 mmol) was added to the reaction mixture at RT and stirred for 16 h at 65° C. Progress of the reaction was monitored by LCMS. After completion of the reaction, the reaction mixture was concentrated under reduced pressure. The residue was diluted with EtOAc and washed with chilled water and a brine solution. Organic layer was dried over anhydrous Na₂SO₄, filtered, and concentrated under reduced pressure to give the crude product. The crude product was purified by column chromatography using silica, 50% EA in pet ether as eluent to afford tert-butyl 4-[[1-(2,6-dioxo-3-piperidyl)-2-oxo-benzo[cd]indol-6-yl]amino]piperidine-1-carboxylate Compound 65 (0.2 g, 409.58 μmol, 43.00% yield, 98% purity) as brown color solid. TLC: 50% EA in pet ether (Rf: 0.3).

### Example 51. Synthesis of 3-[2-oxo-6-(4-piperidylamino)benzo[cd]indol-1-yl]piperidine-2,6-dione trifluoroacetate (Compound 66)

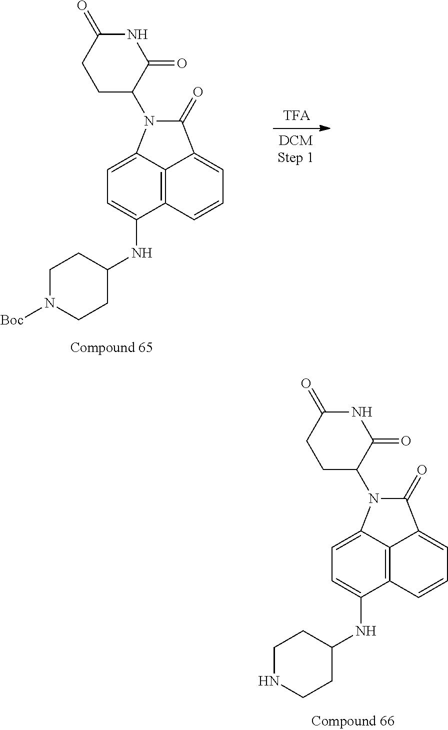 [SMILES: CC(C)(C)OC(=O)N1CCC(Nc2ccc3c4c(cccc24)C(=O)N3C2CCC(=O)NC2=O)CC1 ; O=C1CCC(N2C(=O)c3cccc4c(NC5CCNCC5)ccc2c34)C(=O)N1]

Step 1: To a stirred a suspension of tert-butyl 4-[[1-(2,6-dioxo-3-piperidyl)-2-oxo-benzo[cd]indol-6-yl]amino]piperidine-1-carboxylate Compound 65 (0.1 g, 208.97 μmol, 000) in DCM (5 mL) is added trifluoroacetic acid (238.27 mg, 2.09 mmol, 161.00 μL) dropwise. The reaction mixture was stirred for 3 h at room temperature. The reaction mixture was concentrated under vacuum and the residue was triturated with diethyl ether (10 mL) to obtain 3-[2-oxo-6-(4-piperidylamino)benzo[cd]indol-1-yl]piperidine-2,6-dione trifluoroacetate Compound 66 (0.1 g, 168.55 μmol, 80.66% yield, 83% purity) TLC: Rf 0.2 (10% MeOH/DCM)

### Example 52. Synthesis of 9-[4-[[1-(2,6-dioxo-3-piperidyl)-2-oxo-benzo[cd]indol-6-yl]amino]-1-piperidyl]nonanoic acid (Compound 67)

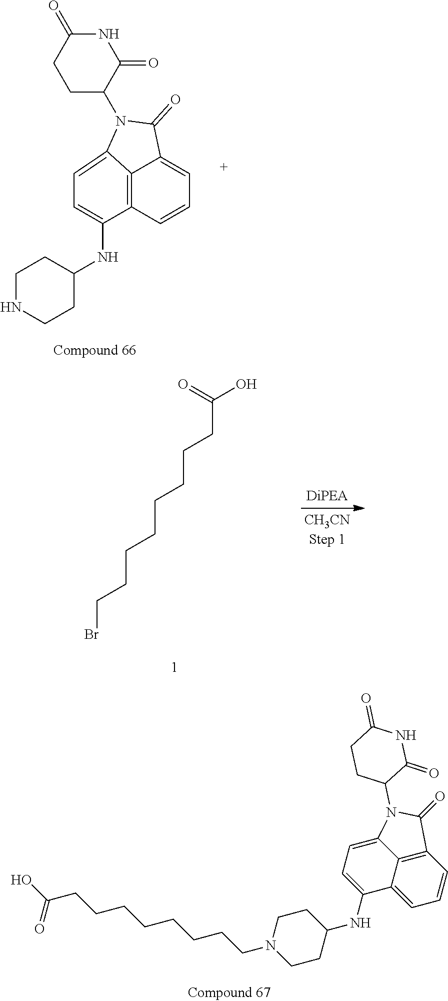 [SMILES: O=C1CCC(N2C(=O)c3cccc4c(NC5CCNCC5)ccc2c34)C(=O)N1 ; O=C(O)CCCCCCCCBr ; O=C(O)CCCCCCCCN1CCC(Nc2ccc3c4c(cccc24)C(=O)N3C2CCC(=O)NC2=O)CC1]

Step 1: A solution of 3-[2-oxo-6-(4-piperidylamino)benzo[cd]indol-1-yl]piperidine-2,6-dione Compound 66 (50 mg, 101.53 μmol), DIPEA (78.74 mg, 609.20 μmol, 106.11 μL) and 9-bromononanoic acid 1 (24.08 mg, 101.53 μmol) in ACN (4 mL) was stirred at 80° C. for 24 h in a sealed tube. The reaction mixture was concentrated under vacuum and the residue was triturated with diethyl ether (10 mL) to obtain 9-[4-[[1-(2,6-dioxo-3-piperidyl)-2-oxo-benzo[cd]indol-6-yl]amino]-1-piperidyl]nonanoic acid Compound 67 (50 mg, 66.40 μmol, 65.40% yield, 71% purity). TLC: Rf 0.2 (70% EtOAc/Pet ether)

### Example 53. Synthesis of 3-[7-(difluoromethyl)-6-(1-methylpyrazol-4-yl)-3,4-dihydro-2H-quinolin-1-yl]-1-[1-[9-[4-[[1-(2,6-dioxo-3-piperidyl)-2-oxo-benzo[cd]indol-6-yl]amino]-1-piperidyl]nonanoyl]-4-piperidyl]-N-methyl-6,7-dihydro-4H-pyrazolo[4,3-c]pyridine-5-carboxamide (Compound 68)

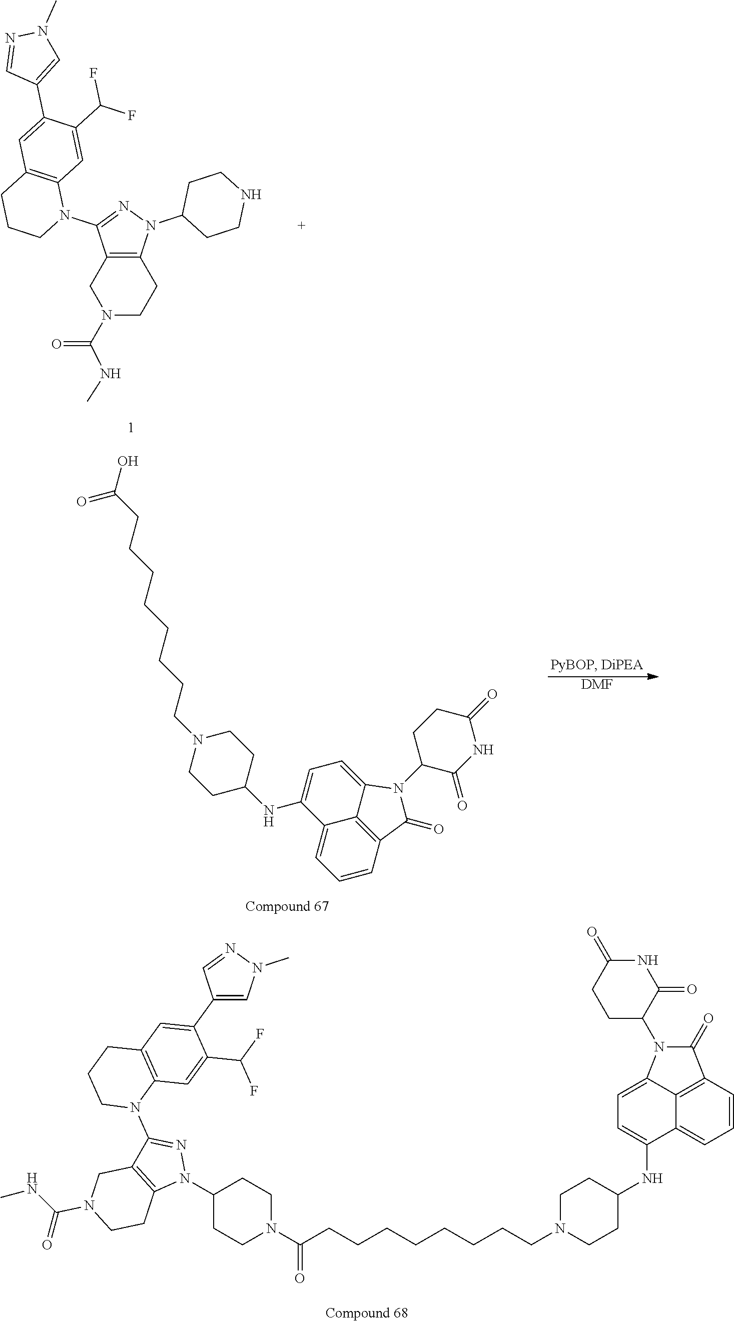

Step 1: To a stirred solution of 3-[7-(difluoromethyl)-6-(1-methylpyrazol-4-yl)-3,4-dihydro-2H-quinolin-1-yl]-N-methyl-1-(4-piperidyl)-6,7-dihydro-4H-pyrazolo[4,3-c]pyridine-5-carboxamide 1 (WO2020173440, 50 mg, 89.12 μmol) and 9-[4-[[1-(2,6-dioxo-3-piperidyl)-2-oxo-benzo[cd]indol-6-yl]amino]-1-piperidyl]nonanoic acid Compound 67 (47.65 mg, 89.12 μmol) in DMF (2 mL) is added N-ethyl-N-isopropyl-propan-2-amine (69.11 mg, 534.69 μmol, 93.13 μL). The resulting solution stirred for 5 mins at RT then benzotriazol-1-yloxy(tripyrrolidin-1-yl)phosphonium;hexafluorophosphate (69.56 mg, 133.67 μmol) added and the reaction mixture was stirred at 28° C. for 16 hr. The reaction mixture was concentrated and the crude material was purified by HPLC to afford 3-[7-(difluoromethyl)-6-(1-methylpyrazol-4-yl)-3,4-dihydro-2H-quinolin-1-yl]-1-[1-[9-[4-[[1-(2,6-dioxo-3-piperidyl)-2-oxo-benzo[cd]indol-6-yl]amino]-1-piperidyl]nonanoyl]-4-piperidyl]-N-methyl-6,7-dihydro-4H-pyrazolo[4,3-c]pyridine-5-carboxamide Compound 68 (19.35 mg, 17.44 μmol, 19.57% yield, 98.00% purity). LCMS m/z [M+H]⁺=1041.1

### Example 54. Synthesis of 3-(6-bromo-1-oxo-1H-benzo[de]isoquinolin-2(3H)-yl)piperidine-2,6-dione (Compound 69) and 3-(7-bromo-1-oxo-1H-benzo[de]isoquinolin-2(3H)-yl)piperidine-2,6-dione (Compound 70)

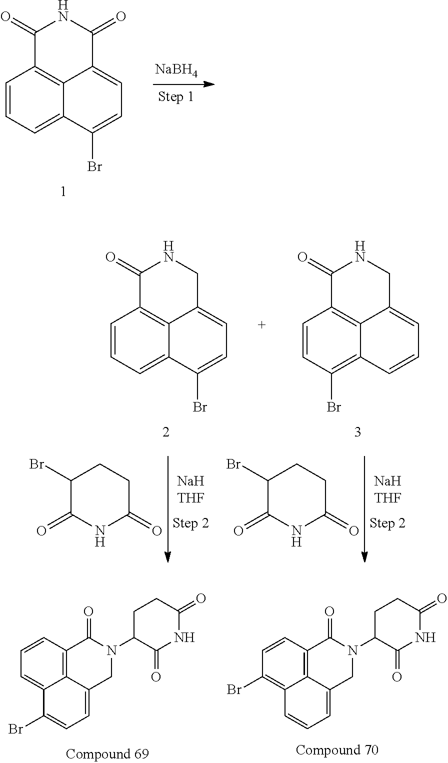

Step 1: To a solution of 6-bromo-1H-benzo[de]isoquinoline-1,3(2H)-dione 1 in an appropriate solvent is added sodium borohydride in a portionwise manner. Once the reaction is judged to be complete, standard workup and purification protocols afford the two regioisomers 6-bromo-2,3-dihydro-1H-benzo[de]isoquinolin-1-one 2 and 7-bromo-2,3-dihydro-1H-benzo[de]isoquinolin-1-one 3.

Step 2: To a solution of 6-bromo-2,3-dihydro-1H-benzo[de]isoquinolin-1-one 2 or 7-bromo-2,3-dihydro-1H-benzo[de]isoquinolin-1-one 3 (1 eq) in THE at 0° C. is added NaH (60% in oil, 10 eq), in portions, at 0° C. and the reaction mixture is stirred at room temperature for 60 min. The reaction mixture is cooled to 0° C., 3-bromopiperidine-2,6-dione (5 eq) in THE is added slowly, the cooling bath removed, and the reaction mixture is slowly heated to 65° C. The reaction mixture is stirred at this temperature until the reaction is judged complete. A standard workup and purification using standard protocols affords 3-(6-bromo-1-oxo-1H-benzo[de]isoquinolin-2(3H)-yl)piperidine-2,6-dione (Compound 69) or 3-(7-bromo-1-oxo-1H-benzo[de]isoquinolin-2(3H)-yl)piperidine-2,6-dione (Compound 70) respectively.

### Example 55. Synthesis of 1-(5-bromo-2-oxo-1,2-dihydroacenaphthylen-1-yl)dihydropyrimidine-2,4(1H,3H)-dione (Compound 71)

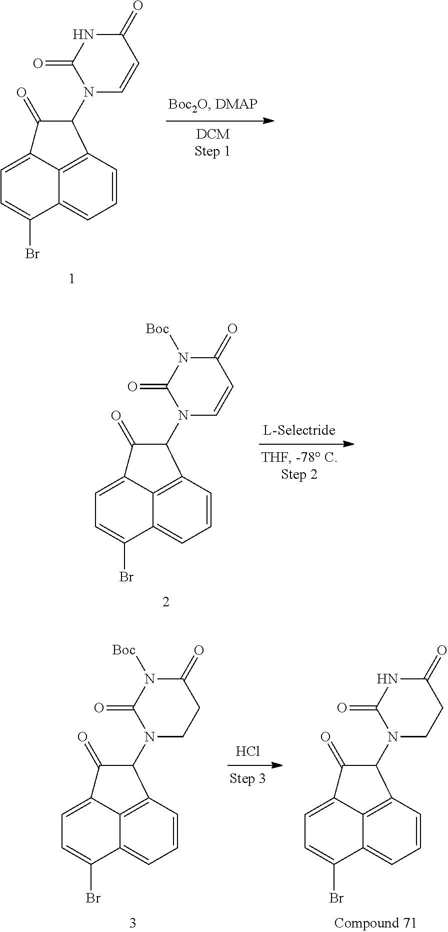 [SMILES: O=C1c2ccc(Br)c3cccc(c23)C1n1ccc(=O)[nH]c1=O ; CC(C)(C)OC(=O)n1c(=O)ccn(C2C(=O)c3ccc(Br)c4cccc2c34)c1=O ; CC(C)(C)OC(=O)N1C(=O)CCN(C2C(=O)c3ccc(Br)c4cccc2c34)C1=O ; Cl ; O=C1CCN(C2C(=O)c3ccc(Br)c4cccc2c34)C(=O)N1]

Step 1: To a solution of 1-(5-bromo-2-oxo-1,2-dihydroacenaphthylen-1-yl)pyrimidine-2,4(1H,3H)-dione 1 in DCM is added DMAP and Boc₂O and stirred until the reaction is judged complete. A standard workup and purification using standard protocols affords tert-butyl 3-(5-bromo-2-oxo-1,2-dihydroacenaphthylen-1-yl)-2,6-dioxo-3,6-dihydropyrimidine-1(2H)-carboxylate 2.

Step 2: To a solution of tert-butyl 3-(5-bromo-2-oxo-1,2-dihydroacenaphthylen-1-yl)-2,6-dioxo-3,6-dihydropyrimidine-1(2H)-carboxylate 2 in THF at −78° C. is added L-selectride dropwise. The reaction is stirred at the appropriate temperature until the reaction is judged complete. A standard workup and purification using standard protocols affords tert-butyl 3-(5-bromo-2-oxo-1,2-dihydroacenaphthylen-1-yl)-2,6-dioxotetrahydropyrimidine-1(2H)-carboxylate 3.

Step 3: To a stirred solution of tert-butyl 3-(5-bromo-2-oxo-1,2-dihydroacenaphthylen-1-yl)-2,6-dioxotetrahydropyrimidine-1(2H)-carboxylate 3 in the appropriate solvent is added concentrated hydrochloric acid. The reaction is stirred at the appropriate temperature until the reaction is judged complete. A standard workup and purification using standard protocols affords 1-(5-bromo-2-oxo-1,2-dihydroacenaphthylen-1-yl)dihydropyrimidine-2,4(1H,3H)-dione (Compound 71).

### Example 56. Synthesis of 1-(5-bromo-2-oxo-1,2-dihydroacenaphthylen-1-yl)pyrimidine-2,4(1H,3H)-dione (Compound 72)

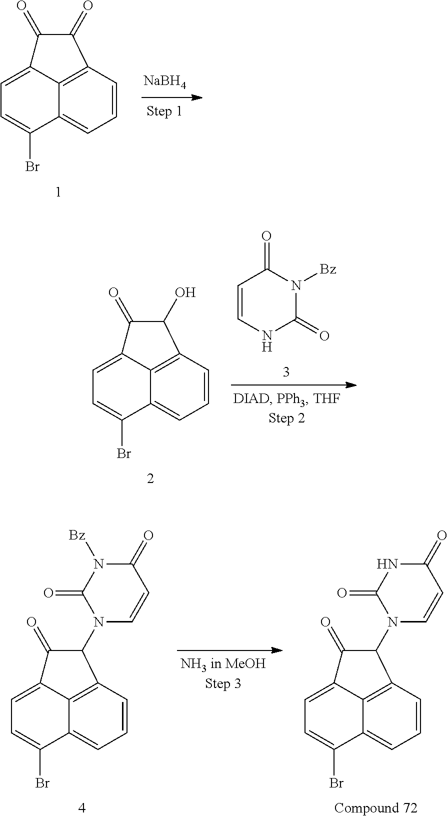

Step 1: To a solution of 6-bromo-1H-benzo[de]isoquinoline-1,3(2H)-dione 1 in an appropriate solvent is added sodium borohydride in a portionwise manner. Once the reaction is judged to be complete, standard workup and purification protocols affords 6-bromo-2-hydroxyacenaphthylen-1(2H)-one 2.

Step 2: To a stirred solution of 6-bromo-2-hydroxyacenaphthylen-1(2H)-one 2 in THE is added DIAD, PPh₃, and 3-benzoylpyrimidine-2,4(1H,3H)-dione 3. The reaction mixture is then stirred at the appropriate temperature until the reaction is judged complete. A standard workup and purification using standard protocols affords 3-benzoyl-1-(5-bromo-2-oxo-1,2-dihydroacenaphthylen-1-yl)pyrimidine-2,4(1H,3H)-dione 4.

Step 3: In a reaction vessel, 3-benzoyl-1-(5-bromo-2-oxo-1,2-dihydroacenaphthylen-1-yl)pyrimidine-2,4(1H,3H)-dione 4 is dissolved in a solution of NH₃ in MeOH and stirred at the appropriate temperature until the reaction is judged complete. A standard workup and purification using standard protocols affords 1-(5-bromo-2-oxo-1,2-dihydroacenaphthylen-1-yl)pyrimidine-2,4(1H,3H)-dione (Compound 72).

### Example 57. Synthesis of 3-(6-bromo-2-oxo-1,4,4a-triazacyclopenta[cd]inden-1(2H)-yl)piperidine-2,6-dione (Compound 73) and 3-(6-bromo-1-oxo-2,4,4a-triazacyclopenta[cd]inden-2(1H)-yl)piperidine-2,6-dione (Compound 74)

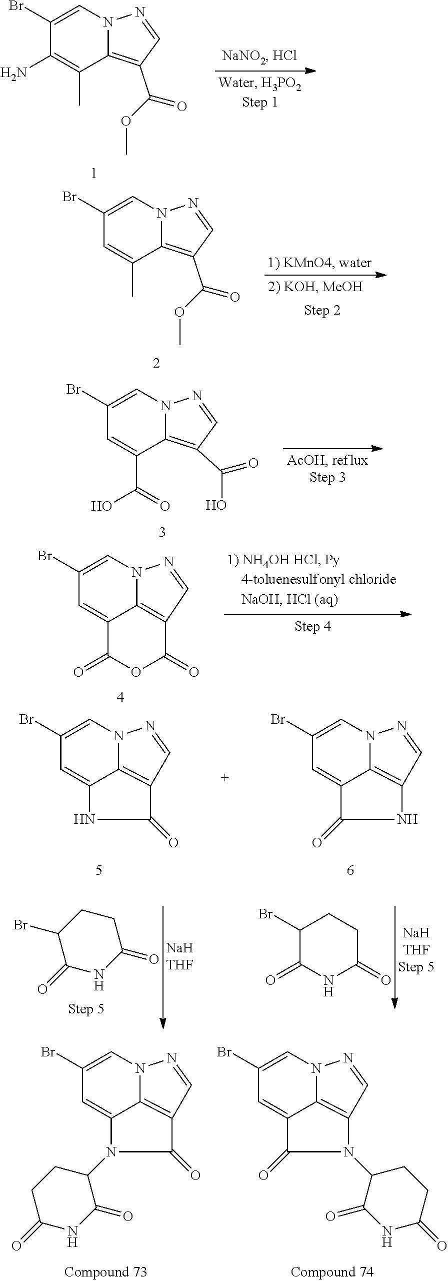

Step 1: To a suspension of methyl 5-amino-6-bromo-4-methylpyrazolo[1,5-a]pyridine-3-carboxylate 1 (synthesized as described in WO2017178377) in aqueous hydrochloric acid is added sodium nitrite. After the appropriate amount of time, an aqueous solution of H₃PO₂ is added dropwise and the reaction solution is again stirred until the reaction is judged to be complete. A standard workup and purification using standard protocols affords methyl 6-bromo-4-methylpyrazolo[1,5-a]pyridine-3-carboxylate 2.

Step 2: To a suspension of methyl 6-bromo-4-methylpyrazolo[1,5-a]pyridine-3-carboxylate 2 in water is added potassium permanganate. After the reaction is judged to be complete, standard workup and purification using standard protocols affords a crude material used immediately by dissolving in methanol. Potassium hydroxide is then added to the resulting solution and the mixture is heated until the reaction is judged to be complete. methyl 6-bromo-4-methylpyrazolo[1,5-a]pyridine-3-carboxylate 2. A standard workup and purification using standard protocols affords 6-bromopyrazolo[1,5-a]pyridine-3,4-dicarboxylic acid 3.

Step 3: 6-bromopyrazolo[1,5-a]pyridine-3,4-dicarboxylic acid 3 is dissolved in acetic acid and heated to reflux until the reaction is judged to be complete. A standard workup and purification using standard protocols affords 4-bromo-6H,8H-7-oxa-2,2a-diazaacenaphthylene-6,8-dione 4.

Step 4: A solution of 4-bromo-6H,8H-7-oxa-2,2a-diazaacenaphthylene-6,8-dione 4 (1 eq.) and hydroxylamine hydrochloride (1 eq.) in pyridine (10 vol eq.) is heated at reflux for 5 h, followed by cooling to 80° C. and the addition of 4-toluenesulfonyl chloride (2 eq). After addition, the temperature is raised and the reaction stirred at reflux for 5 h, followed by cooling. The reaction mixture is poured into water and extracted with EtOAc (3×). The organic layers are combined, washed with water, sat. aq. NaHCO₃, brine, and dried over anhydrous Na₂SO₄, then filtered and evaporated to dryness. To a stirred solution of the residue in EtOH (10 vol eq) and water (10 vol eq) is added 1M aqueous sodium hydroxide (10 eq) dropwise. Thereafter, the mixture is stirred at reflux for 3 h while distilling off the ethanol. After completion of the reaction, the reaction mixture is cooled to 75° C., and hydrochloric acid, 36% w/w aq. soln. (10 vol eq) is added dropwise. Standard work up and purification followed by separation of regioisomers affords 6-bromo-1,4,4a-triazacyclopenta[cd]inden-2(1H)-one 5 and 6-bromo-2,4,4a-triazacyclopenta[cd]inden-1(2H)-one 6.

Step 5: To a solution of 6-bromo-1,4,4a-triazacyclopenta[cd]inden-2(1H)-one 5 or 6-bromo-2,4,4a-triazacyclopenta[cd]inden-1(2H)-one 6 in THE at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide (5-8 eq) is added in portions before the cooling bath is once again removed and the reaction slowly heated to 70° C. until the reaction is judged complete. A standard workup and purification using standard protocols affords 3-(6-bromo-2-oxo-1,4,4a-triazacyclopenta[cd]inden-1(2H)-yl)piperidine-2,6-dione Compound 73 or 3-(6-bromo-1-oxo-2,4,4a-triazacyclopenta[cd]inden-2(1H)-yl)piperidine-2,6-dione Compound 74, respectively.

### Example 58. Synthesis of 3-(6-bromo-4-methyl-2-oxo-2,4-dihydro-1H-pyrrolo[4,3,2-cd]indol-1-yl)piperidine-2,6-dione (Compound 75) and 3-(6-bromo-4-methyl-1-oxo-1,4-dihydro-2H-pyrrolo[2,3,4-cd]indol-2-yl)piperidine-2,6-dione (Compound 76)

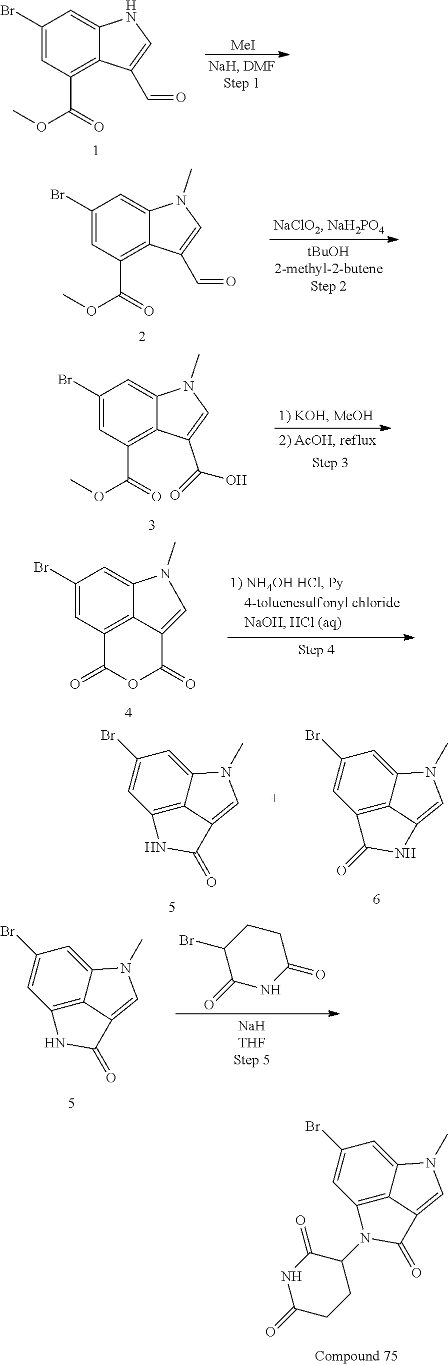

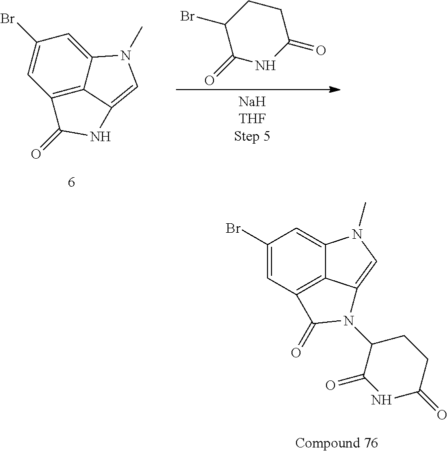 [SMILES: Cn1cc2c3c(cc(Br)cc31)C(=O)N2 ; O=C1CCC(Br)C(=O)N1 ; Cn1cc2c3c(cc(Br)cc31)C(=O)N2C1CCC(=O)NC1=O]

Step 1: To a stirred solution of methyl 6-bromo-3-formyl-1H-indole-4-carboxylate 1 (synthesized as described in Bioorg. Med. Chem. Lett. (2017) 27(2) 217-222) in DMF is added sodium hydride in portions at 0° C. Following addition, methyl iodide is added dropwise then the reaction is heated as necessary. Once the reaction is judged to be complete, a standard workup and purification using standard protocols affords methyl 6-bromo-3-formyl-1-methyl-1H-indole-4-carboxylate 2.

Step 2: To a stirred solution of methyl 6-bromo-3-formyl-1-methyl-1H-indole-4-carboxylate 2 in tert-butylalcohol is added NaClO₂, NaH₂PO₄, and 2-methyl-2-butene. The resulting solution is stirred until the reaction is judged to be complete. A standard workup and purification using standard protocols affords 6-bromo-4-(methoxycarbonyl)-1-methyl-1H-indole-3-carboxylic acid 3.

Step 3: In a reaction flask, 6-bromo-4-(methoxycarbonyl)-1-methyl-1H-indole-3-carboxylic acid 3 is dissolved in methanol. Potassium hydroxide is then added to the resulting solution and the mixture is heated until the reaction is judged to be complete. A standard workup and purification using standard protocols affords material used immediately in the next transformation. This material is dissolved in acetic acid and heated to reflux until the reaction is judged to be complete. A standard workup and purification using standard protocols affords 7-bromo-1-methyl-3H-pyrano[3,4,5-cd]indole-3,5(1H)-dione 4.

Step 4: A solution of 7-bromo-1-methyl-3H-pyrano[3,4,5-cd]indole-3,5(1H)-dione 4 (1 eq.) and hydroxylamine hydrochloride (1 eq.) in pyridine (10 vol eq.) is heated at reflux for 5 h, followed by cooling to 80° C. and the addition of 4-toluenesulfonyl chloride (2 eq). After addition, the temperature is raised and the reaction stirred at reflux for 5 h, followed by cooling. The reaction mixture is poured into water and extracted with EtOAc (3×). The organic layers are combined, washed with water, sat. aq. NaHCO₃, brine, and dried over anhydrous Na₂SO₄, then filtered and evaporated to dryness. To a stirred solution of the residue in EtOH (10 vol eq) and water (10 vol eq) is added 1M aqueous sodium hydroxide (10 eq) dropwise. Thereafter, the mixture is stirred at reflux for 3 h while distilling off the ethanol. After completion of the reaction, the reaction mixture is cooled to 75° C., and hydrochloric acid, 36% w/w aq. soln. (10 vol eq) is added dropwise. Standard work up and purification followed by separation of regioisomers affords 6-bromo-4-methyl-1,4-dihydro-2H-pyrrolo[4,3,2-cd]indol-2-one 5 and 6-bromo-4-methyl-2,4-dihydro-1H-pyrrolo[2,3,4-cd]indol-1-one 6.

Step 5: To a solution of 6-bromo-4-methyl-1,4-dihydro-2H-pyrrolo[4,3,2-cd]indol-2-one 5 or 6-bromo-4-methyl-2,4-dihydro-1H-pyrrolo[2,3,4-cd]indol-1-one 6 in THE at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide (5-8 eq) is added in portions before the cooling bath is once again removed and the reaction slowly heated to 70° C. until the reaction is judged complete. A standard workup and purification using standard protocols affords 3-(6-bromo-4-methyl-2-oxo-2,4-dihydro-1H-pyrrolo[4,3,2-cd]indol-1-yl)piperidine-2,6-dione Compound 75 or 3-(6-bromo-4-methyl-1-oxo-1,4-dihydro-2H-pyrrolo[2,3,4-cd]indol-2-yl)piperidine-2,6-dione Compound 76, respectively.

### Example 59. Synthesis of 3-(3-chloro-7-oxopyrrolo[2,3,4-de]phthalazin-8(7H)-yl)piperidine-2,6-dione (Compound 77)

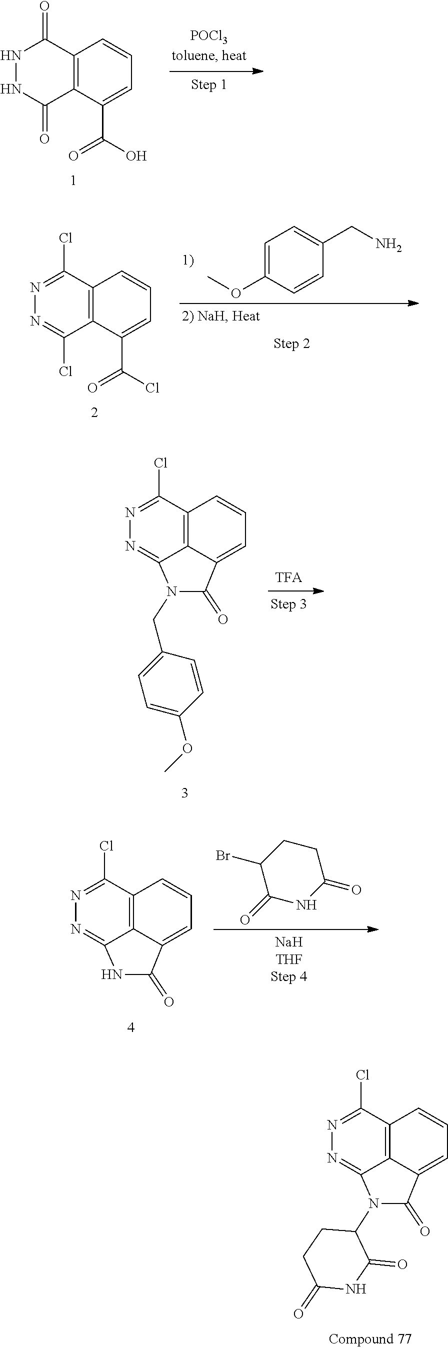

Step 1: To a solution of 1,4-dioxo-1,2,3,4-tetrahydrophthalazine-5-carboxylic acid 1 in toluene is added POCl₃ dropwise then the reaction mixture heated as appropriate. Once the reaction is judged to be complete, a standard workup and purification using standard protocols affords 1,4-dichlorophthalazine-5-carbonyl chloride 2.

Step 2: To a solution of 1,4-dichlorophthalazine-5-carbonyl chloride 2 in the appropriate solvent is added 4-methoxybenzylamine and the resulting mixture is stirred until the reaction is judged to be complete. The reaction solution is cooled to 0° C. and sodium hydride is added portionwise. Following addition of sodium hydride, the reaction is heated as necessary until the reaction is judged to be complete. A standard workup and purification using standard protocols affords 3-chloro-8-(4-methoxybenzyl)pyrrolo[2,3,4-de]phthalazin-7(8H)-one 3.

Step 3: A solution of 3-chloro-8-(4-methoxybenzyl)pyrrolo[2,3,4-de]phthalazin-7(8H)-one 3 dissolved in trifluoroacetic acid is stirred until the reaction is judged to be complete. A standard workup and purification using standard protocols affords 3-chloropyrrolo[2,3,4-de]phthalazin-7(8H)-one 4.

Step 4: To a solution of 3-chloropyrrolo[2,3,4-de]phthalazin-7(8H)-one 4 in THE at 0° C. is added NaH (60% dispersion in mineral oil, 10-15 eq) in portions. The cooling bath is then removed and the reaction mixture is stirred at this temperature for 1 hr. The reaction mixture is re-cooled to 0° C. and 3-bromo-glutarimide (5-8 eq) is added in portions before the cooling bath is once again removed and the reaction slowly heated to 70° C. until the reaction is judged complete. A standard workup and purification using standard protocols affords 3-(3-chloro-7-oxopyrrolo[2,3,4-de]phthalazin-8(7H)-yl)piperidine-2,6-dione Compound 77.

### Example 60. Synthesis of 3-(5-bromo-2-methoxyacenaphthylen-1-yl)piperidine-2,6-dione (Compound 78)

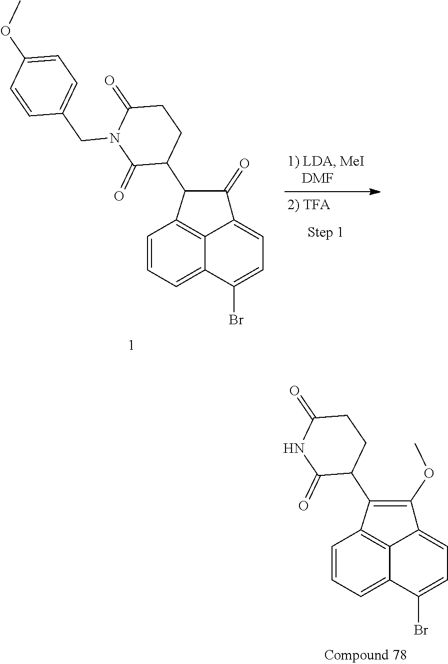 [SMILES: COc1ccc(CN2C(=O)CCC(C3C(=O)c4ccc(Br)c5cccc3c45)C2=O)cc1 ; COC1=C(C2CCC(=O)NC2=O)c2cccc3c(Br)ccc1c23]

Step 1: 3-(5-Bromo-2-oxo-1,2-dihydroacenaphthylen-1-yl)-1-(4-methoxybenzyl)piperidine-2,6-dione 1 is dissolved in DMF and brought to −78° C. using an ice bath. LDA is added dropwise and the reaction is allowed to stir. Mel is added and the reaction is allowed to stir at room temperature until judged to be complete. Intermediate 3-(5-bromo-2-methoxyacenaphthylen-1-yl)-1-(4-methoxybenzyl)piperidine-2,6-dione is obtained following standard workup and purification protocols and then dissolved in TFA and allowed to stir until reaction is judged to be complete. 3-(5-Bromo-2-methoxyacenaphthylen-1-yl)piperidine-2,6-dione Compound 78 is obtained following standard workup and purification protocols.

### Example 61. Synthesis of (E)-3-(5-bromo-2-oxoacenaphthylen-1(2H)-ylidene)piperidine-2,6-dione (Compound 79)

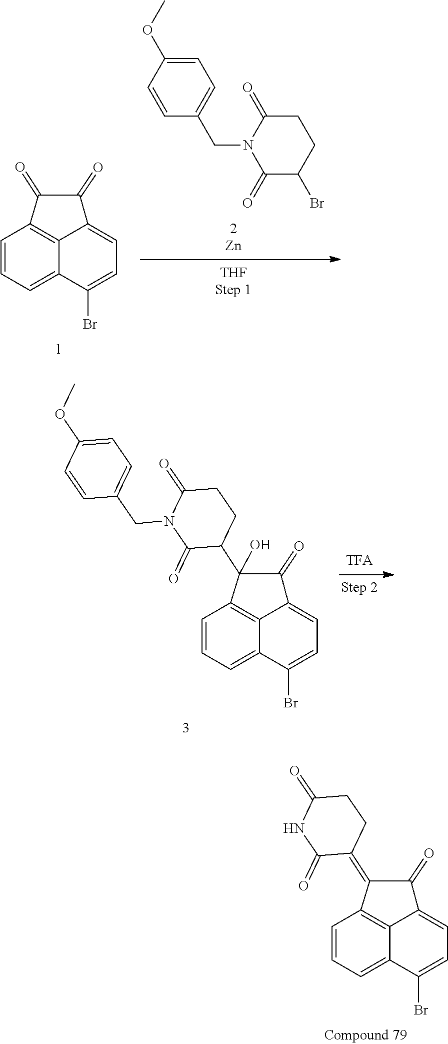 [SMILES: O=C1C(=O)c2ccc(Br)c3cccc1c23 ; COc1ccc(CN2C(=O)CCC(Br)C2=O)cc1 ; COc1ccc(CN2C(=O)CCC(C3(O)C(=O)c4ccc(Br)c5cccc3c45)C2=O)cc1 ; O=C1CC/C(=c2\c(=O)c3ccc(Br)c4cccc2c43)C(=O)N1]

Step 1: 3-Bromo-1-(4-methoxybenzyl)piperidine-2,6-dione 2 in THE is added dropwise to a suspension of Zn in THE and cooled to 0° C. in an ice bath. The suspension is stirred for 1 hour. 5-Bromoacenaphthylene-1,2-dione 1 dissolved in THE is added dropwise to the suspension and the reaction is stirred until judged to be compete. 3-(5-Bromo-1-hydroxy-2-oxo-1,2-dihydroacenaphthylen-1-yl)-1-(4-methoxybenzyl)piperidine-2,6-dione 3 is obtained following standard workup and purification protocols.

Step 2: 3-(5-Bromo-1-hydroxy-2-oxo-1,2-dihydroacenaphthylen-1-yl)-1-(4-methoxybenzyl)piperidine-2,6-dione 3 is dissolved in TFA and allowed to stir until the reaction is judged to be complete. (E)-3-(5-Bromo-2-oxoacenaphthylen-1(2H)-ylidene)piperidine-2,6-dione Compound 79 is obtained following standard workup and purification protocols.

### Example 62. Synthesis of 1-(6-bromo-2-oxobenzo[cd]indol-1(2H)-yl)dihydropyrimidine-2,4(1H,3H)-dione (Compound 80)

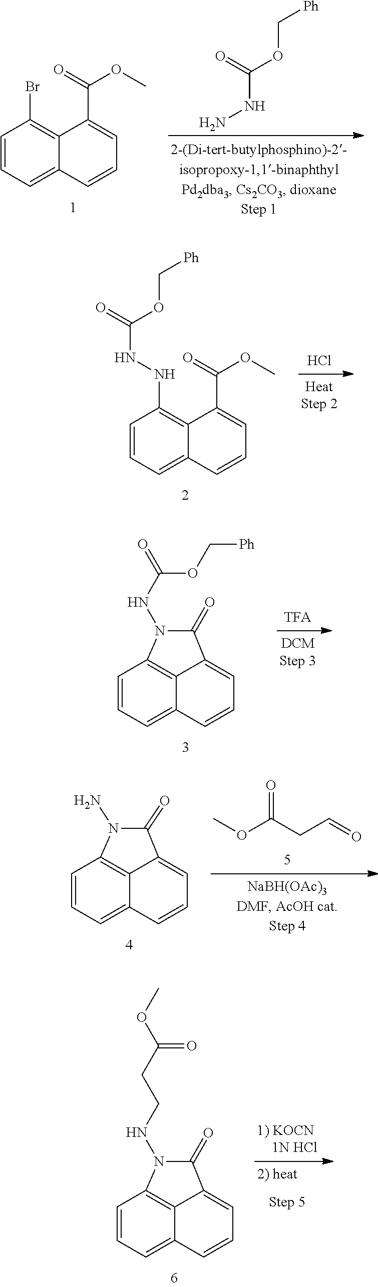

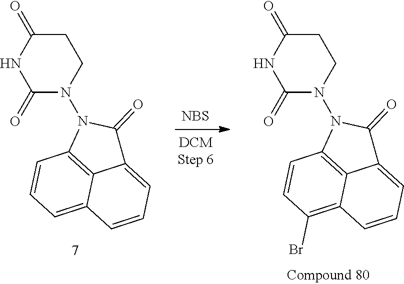

Step 1: Methyl 8-bromo-1-naphthoate 1 and benzyl hydrazinecarboxylate 2 are dissolved in dioxane and 2-(di-tert-butylphosphino)-2′-isopropoxy-1,1′-binaphthyl Pd₂dba₃ and Cs₂CO₃ are added. The solution is stirred until judged to be complete. Benzyl 2-(8-(methoxycarbonyl)naphthalen-1-yl)hydrazine-1-carboxylate 2 is obtained following standard workup and purification protocols.

Step 2: Benzyl 2-(8-(methoxycarbonyl)naphthalen-1-yl)hydrazine-1-carboxylate 2 is dissolved in a suitable solvent and HCl is added. The reaction is heated and allowed to stir until judged to be complete. Benzyl (2-oxobenzo[cd]indol-1(2H)-yl)carbamate 3 is obtained following standard workup and purification protocols.

Step 3: Benzyl (2-oxobenzo[cd]indol-1(2H)-yl)carbamate 3 is dissolved in DCM and TFA is added. The solution is stirred until judged to be complete. 1-Aminobenzo[cd]indol-2(1H)-one 4 is obtained following standard workup and purification protocols.

Step 4: 1-Aminobenzo[cd]indol-2(1H)-one 4, methyl 3-oxopropanoate 5, NaBH(OAc)₃, and AcOH are dissolved in DMF and allowed to stir at room temperature until judged to be complete. Methyl 3-((2-oxobenzo[cd]indol-1(2H)-yl)amino)propanoate 6 is obtained following standard workup and purification protocols.

Step 5: Methyl 3-((2-oxobenzo[cd]indol-1(2H)-yl)amino)propanoate 6 is dissolved in suitable solvent and KOCN and 1N HCl are added. The reaction is heated and allowed to stir until judged to be complete. 1-(2-Oxobenzo[cd]indol-1(2H)-yl)dihydropyrimidine-2,4(1H,3H)-dione 7 is obtained following standard workup and purification protocols.

Step 6: To a solution of 1-(2-oxobenzo[cd]indol-1(2H)-yl)dihydropyrimidine-2,4(1H,3H)-dione 7 in DCM at 0° C. is added NBS. The cooling bath is removed and the reaction mixture is stirred at room temperature until judged to be complete. Workup and purification using standard protocols affords 1-(6-bromo-2-oxobenzo[cd]indol-1(2H)-yl)dihydropyrimidine-2,4(1H,3H)-dione Compound 80.

### Example 63. Synthesis of 3-(5-bromoacenaphthylen-1-yl)piperidine-2,6-dione (Compound 81)

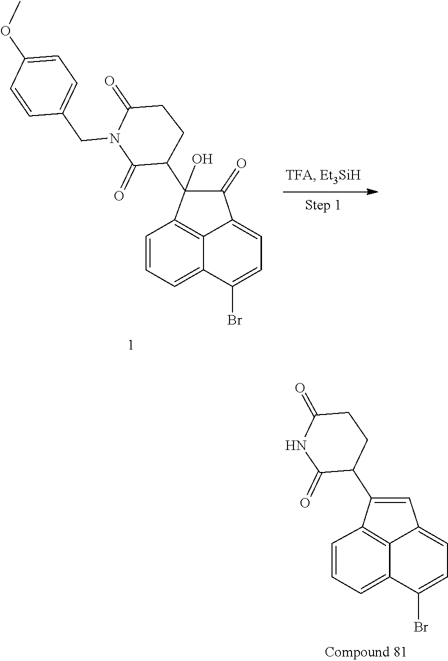 [SMILES: COc1ccc(CN2C(=O)CCC(C3(O)C(=O)c4ccc(Br)c5cccc3c45)C2=O)cc1 ; O=C1CCC(C2=Cc3ccc(Br)c4cccc2c34)C(=O)N1]

Step 1: 3-(5-Bromo-1-hydroxy-2-oxo-1,2-dihydroacenaphthylen-1-yl)-1-(4-methoxybenzyl)piperidine-2,6-dione 1 is dissolved in TFA and Et₃SiH is added. The reaction is heated and allowed to stir until judged to be complete. 3-(5-Bromoacenaphthylen-1-yl)piperidine-2,6-dione Compound 81 is obtained following standard workup and purification protocols.

### Example 64. Synthesis of 3-(8-bromo-4-oxopyrrolo[4,3,2-de]cinnolin-5(4H)-yl)piperidine-2,6-dione (Compound 82)

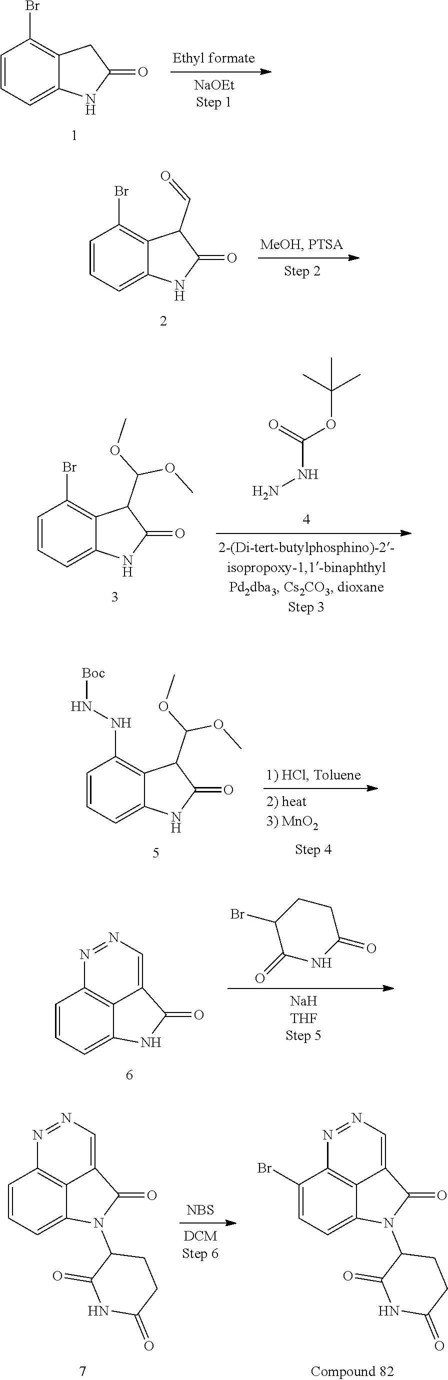

Step 1: 4-Bromoindolin-2-one 1 is dissolved in a suitable solvent and ethyl formate and NaOEt are added. The reaction is allowed to stir until judged to be complete. 4-Bromo-2-oxoindoline-3-carbaldehyde 2 is obtained following standard workup and purification protocols.

Step 2: 4-Bromo-2-oxoindoline-3-carbaldehyde 2 is dissolved in MeOH and p-toluenesulfonic acid is added. The reaction is allowed to stir until judged to be complete. 4-Bromo-3-(dimethoxymethyl)indolin-2-one 3 is obtained following standard workup and purification protocols.

Step 3: 4-Bromo-3-(dimethoxymethyl)indolin-2-one 3 and tert-butyl hydrazinecarboxylate 4 are dissolved in dioxane and 2-(di-tert-butylphosphino)-2′-isopropoxy-1,1′-binaphthyl Pd₂dba₃ and Cs₂CO₃ are added. The solution is stirred until judged to be complete. tert-Butyl 2-(3-(dimethoxymethyl)-2-oxoindolin-4-yl)hydrazine-1-carboxylate 5 is obtained following standard workup and purification protocols.

Step 4: tert-Butyl 2-(3-(dimethoxymethyl)-2-oxoindolin-4-yl)hydrazine-1-carboxylate 5 is dissolved in toluene and HCl is added. The reaction is heated and allowed to stir until judged to be complete. The reaction is cooled to room temperature and MnO₂ is added and the reaction is allowed to stir until judged to be complete. Pyrrolo[4,3,2-de]cinnolin-4(5H)-one 6 is obtained following standard workup and purification protocols.

Step 5: Pyrrolo[4,3,2-de]cinnolin-4(5H)-one 6 is dissolved in dry THE and the solution is brought to 0° C. before sodium hydride (60% dispersion in mineral oil) is added. The reaction mixture is stirred at ambient temperature. After 1 hour, 3-bromopiperidine-2,6-dione dissolved in dry THE (10 mL) is added at 0° C. The reaction mixture is stirred at 65° C. until judged to be complete. 3-(4-Oxopyrrolo[4,3,2-de]cinnolin-5(4H)-yl)piperidine-2,6-dione 7 is obtained following standard workup and purification protocols.

Step 6: To a solution of 3-(4-oxopyrrolo[4,3,2-de]cinnolin-5(4H)-yl)piperidine-2,6-dione 7 in DCM at 0° C. is added NBS. The cooling bath is removed and the reaction mixture is stirred at room temperature until judged to be complete. Workup and purification using standard protocols affords 3-(8-bromo-4-oxopyrrolo[4,3,2-de]cinnolin-5(4H)-yl)piperidine-2,6-dione Compound 82.

### Example 65. Synthesis of 3-(8-bromo-5-oxopyrrolo[2,3,4-de]cinnolin-4(5H)-yl)piperidine-2,6-dione (Compound 83)

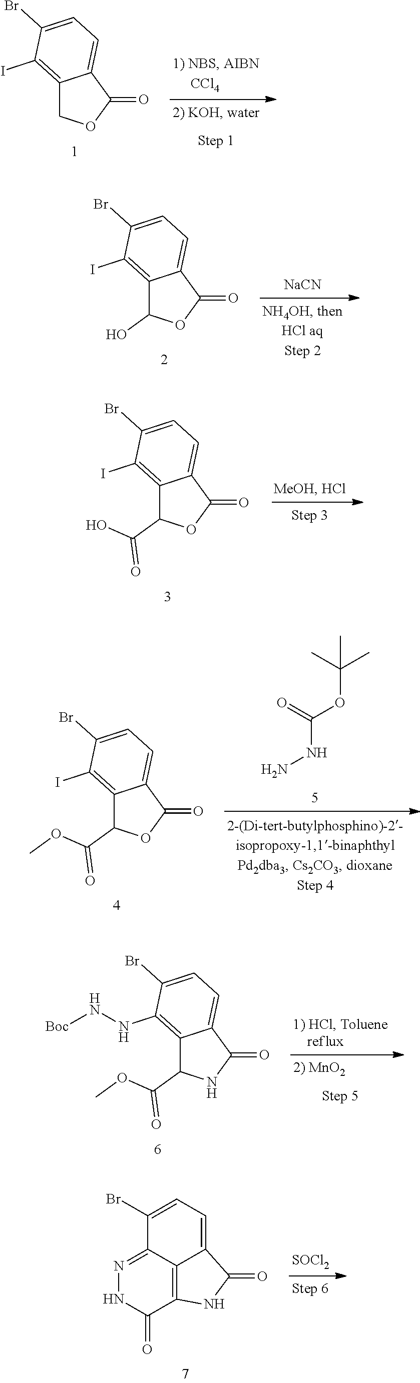

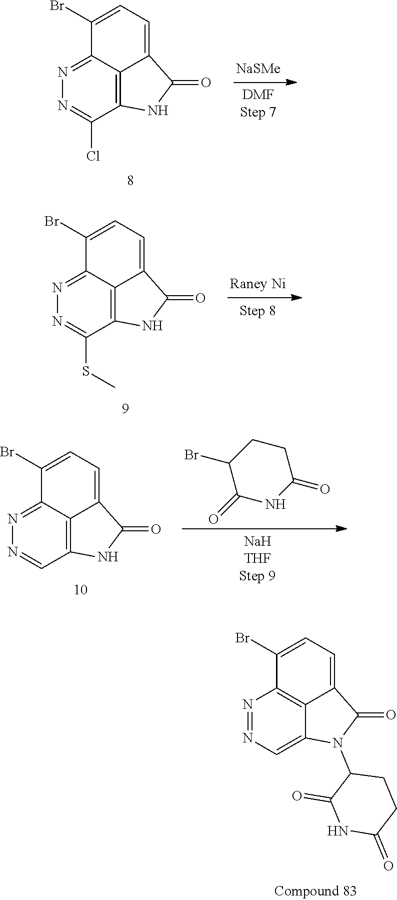

Step 1: 5-Bromo-4-iodoisobenzofuran-1(3H)-one 1 is dissolved in CCl₄ and AIBN and NBS are added. The reaction is allowed to stir until judged to be complete. The reaction is worked up using standard procedures and resulting residue is dissolved in water and KOH is added. Workup and purification using standard protocols affords 5-bromo-3-hydroxy-4-iodoisobenzofuran-1(3H)-one 2.

Step 2: 5-Bromo-3-hydroxy-4-iodoisobenzofuran-1(3H)-one 2 is dissolved in a suitable solvent and NaCN is added followed by NH₄₀H. Aqueous HCl is added and the reaction is stirred. Workup and purification using standard protocols affords 6-bromo-7-iodo-3-oxoisoindoline-1-carboxylic acid 3.

Step 3: 6-Bromo-7-iodo-3-oxoisoindoline-1-carboxylic acid 3 is dissolved in MeOH and HCl is added. The reaction is heated and allowed to stir until judged to be complete. Workup and purification using standard protocols affords methyl 6-bromo-7-iodo-3-oxoisoindoline-1-carboxylate 4.

Step 4: Methyl 6-bromo-7-iodo-3-oxoisoindoline-1-carboxylate 4 and tert-butyl hydrazinecarboxylate 5 are dissolved in dioxane and 2-(di-tert-butylphosphino)-2′-isopropoxy-1,1′-binaphthyl Pd₂dba₃ and Cs₂CO₃ are added. The solution is stirred until judged to be complete. Methyl 6-bromo-7-(2-(tert-butoxycarbonyl)hydrazineyl)-3-oxoisoindoline-1-carboxylate 6 is obtained following standard workup and purification protocols.

Step 5: Methyl 6-bromo-7-(2-(tert-butoxycarbonyl)hydrazineyl)-3-oxoisoindoline-1-carboxylate 6 is dissolved in toluene and HCl is added. The reaction is brought to reflux and allowed to reflux until judged to be complete. The reaction is cooled to room temperature and MnO₂ is added and the reaction is allowed to stir until judged to be complete. 8-Bromo-2,4-dihydropyrrolo[2,3,4-de]cinnoline-3,5-dione 7 is obtained following standard workup and purification protocols.

Step 6: 8-Bromo-2,4-dihydropyrrolo[2,3,4-de]cinnoline-3,5-dione 7 is dissolved in a suitable solvent and brought to 0° C. using an ice bath. SOCl₂ is added and the reaction is allowed to warm to room temperature and stir until judged to be complete. 8-bromo-3-chloropyrrolo[2,3,4-de]cinnolin-5(4H)-one 8 is obtained following standard workup and purification protocols.

Step 7: 8-Bromo-3-chloropyrrolo[2,3,4-de]cinnolin-5(4H)-one 8 is dissolved in DMF and NaSMe is added. The solution is stirred until judged to be complete. 8-Bromo-3-(methylthio)pyrrolo[2,3,4-de]cinnolin-5(4H)-one 9 is obtained following standard workup and purification protocols.

Step 8: 8-Bromo-3-(methylthio)pyrrolo[2,3,4-de]cinnolin-5(4H)-one 9 is dissolved in a suitable solvent and Raney Ni is added. The solution is stirred until judged to be complete. 8-bromopyrrolo[2,3,4-de]cinnolin-5(4H)-one 10 is obtained following standard workup and purification protocols.

Step 9: 8-Bromopyrrolo[2,3,4-de]cinnolin-5(4H)-one 10 is dissolved in dry THE and the solution is brought to 0° C. before sodium hydride (60% dispersion in mineral oil) is added. The reaction mixture is stirred at ambient temperature. After 1 hour, 3-bromopiperidine-2,6-dione dissolved in dry THE (10 mL) is added at 0° C. The reaction mixture is stirred at 65° C. until judged to be complete. 3-(8-Bromo-5-oxopyrrolo[2,3,4-de]cinnolin-4(5H)-yl)piperidine-2,6-dione Compound 83 is obtained following standard workup and purification protocols.

### Example 66

### Synthesis of 2-[4-[1-(2,6-dioxo-3-piperidyl)-2-oxo-benzo[cd]indol-6-yl]-1-piperidyl]acetic acid

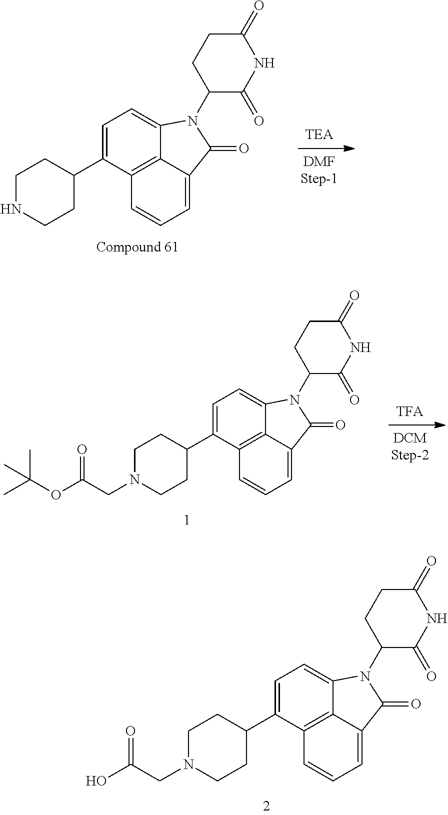 [SMILES: O=C1CCC(N2C(=O)c3cccc4c(C5CCNCC5)ccc2c34)C(=O)N1 ; CC(C)(C)OC(=O)CN1CCC(c2ccc3c4c(cccc24)C(=O)N3C2CCC(=O)NC2=O)CC1 ; O=C(O)CN1CCC(c2ccc3c4c(cccc24)C(=O)N3C2CCC(=O)NC2=O)CC1]

Step-1:

To a stirred solution of 3-[2-oxo-6-(4-piperidyl)benzo[cd]indol-1-yl]piperidine-2,6-dione (Compound 61, 100 mg, 275.17 μmol) and triethyl amine (263.20 mg, 2.60 mmol, 362.54 μL) in N,N-Dimethyl formamide (3 mL) in a sealed tube was added tert-butyl 2-bromoacetate (152.20 mg, 780.32 μmol, 114.44 μL) and the reaction mixture was stirred at room temperature for 16 h. The reaction mixture was poured into cold water (30 ml) and the precipitate was filtered, washed with water and pet ether, and dried to yield tert-butyl 2-[4-[1-(2,6-dioxo-3-piperidyl)-2-oxo-benzo[cd]indol-6-yl]-1-piperidyl]acetate (1, 70 mg, 145.12 μmol, 28% yield) as a light brown solid. LC-MS (ES⁺): m/z 478 [M+H]⁺. ¹H-NMR (400 MHz, DMSO-d6): δ 11.13 (s, 1H), 8.42 (d, J=8.40 Hz, 1H), 8.11 (d, J=6.80 Hz, 1H), 7.86 (t, J=7.20 Hz, 1H), 7.40 (d, J=7.60 Hz, 1H), 7.11 (s, 1H), 5.43-5.47 (m, 1H), 3.39-3.26 (m, 3H), 3.18 (s, 2H), 3.00-2.92 (m, 3H), 2.81-2.74 (m, 1H), 2.71-2.56 (m, 1H), 2.11-2.08 (m, 1H), 1.84-1.76 (m, 4H), 1.45 (s, 9H).

Step-2:

A stirred solution of tert-butyl 2-[4-[1-(2,6-dioxo-3-piperidyl)-2-oxo-benzo[cd]indol-6-yl]-1-piperidyl]acetate (1, 70 mg, 146.58 μmol) in dichloromethane (3 mL) at 0° C. was added trifluoroacetic acid (1.48 g, 12.98 mmol, 1.0 mL) slowly. The reaction mixture was then stirred at room temperature for 2 hours. The reaction mixture was concentrated, the crude material was triturated with diethyl ether, filtered and dried to yield 2-[4-[1-(2,6-dioxo-3-piperidyl)-2-oxo-benzo[cd]indol-6-yl]-1-piperidyl]acetic acid (2, 60 mg, 108.69 μmol, 74% yield) as light brown gummy solid. Used without further purification. LC-MS (ES⁺): m/z 422 [M+H]⁺.

### Example 67

### Synthesis of 4-[4-[1-(2,6-dioxo-3-piperidyl)-2-oxo-benzo[cd]indol-6-yl]-1-piperidyl]butanoic acid

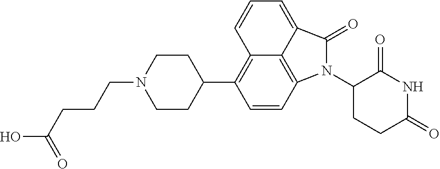 [SMILES: O=C(O)CCCN1CCC(c2ccc3c4c(cccc24)C(=O)N3C2CCC(=O)NC2=O)CC1]

The synthesis of 4-[4-[1-(2,6-dioxo-3-piperidyl)-2-oxo-benzo[cd]indol-6-yl]-1-piperidyl]butanoic acid was substantially similar to 2-[4-[1-(2,6-dioxo-3-piperidyl)-2-oxo-benzo[cd]indol-6-yl]-1-piperidyl]acetic acid, except replacing tert-butyl 2-bromoacetate with tert-butyl 4-bromobutanoate. LC-MS (ES⁺): m/z 450 [M+H]⁺.

### Example 68

### Synthesis of tert-butyl 3-(6-bromo-1,3-benzoxazol-2-yl)azetidine-1-carboxylate

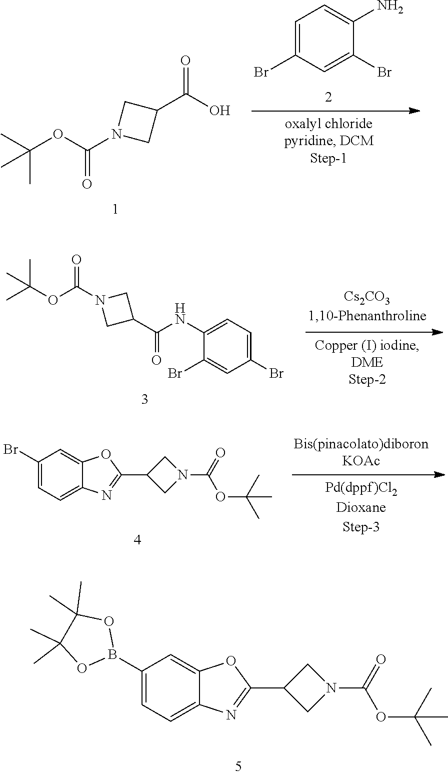 [SMILES: CC(C)(C)OC(=O)N1CC(C(=O)O)C1 ; Nc1ccc(Br)cc1Br ; CC(C)(C)OC(=O)N1CC(C(=O)Nc2ccc(Br)cc2Br)C1 ; CC(C)(C)OC(=O)N1CC(c2nc3ccc(Br)cc3o2)C1 ; CC(C)(C)OC(=O)N1CC(c2nc3ccc(B4OC(C)(C)C(C)(C)O4)cc3o2)C1]

Step 1

To a stirred solution of 1-tert-butoxycarbonylazetidine-3-carboxylic acid (1, 24.06 g, 119.56 mmol) in dichloromethane (200 mL) was added oxalyl chloride (3.34 g, 26.30 mmol, 2.29 mL) and pyridine (2.08 g, 26.30 mmol, 2.13 mL) dropwise at 25° C. and stirred for 2 hr at 25° C. A solution of 2,4-dibromoaniline (2, 6 g, 23.91 mmol, 2.61 mL) in dichloromethane (30 mL) was then added at 25° C. and the reaction mixture was stirred for 16 hours at 25° C. The resulting mixture was quenched with water and extracted with dichloromethane (3×100 mL). The combined organic extracts were washed with water and brine, dried over anhydrous sodium sulphate, filtered and evaporated under reduced pressure to yield tert-butyl 3-[(2,4-dibromophenyl)carbamoyl]azetidine-1-carboxylate (3, 28 g, 23.86 mmol, 99% yield) as an yellow oil. Used without further purification.

Step-2:

To a stirred solution of tert-butyl 3-[(2,4-dibromophenyl)carbamoyl]azetidine-1-carboxylate (3, 27 g, 62.19 mmol) in 1,2 dimethoxy ethane (250 mL) was added cesium carbonate (30.40 g, 93.29 mmol), 1,10-phenanthroline (2.24 g, 12.44 mmol) and copper (I) iodide (1.18 g, 6.22 mmol, 210.76 μL) and the mixture was stirred for 16 hours at 90° C. The reaction mixture was cooled to ambient temperature, diluted with ethyl acetate and filtered through a celite cake; filter cake washed with ethyl acetate. Evaporation of the solvents followed by column chromatography (silica gel, 0 to 20% ethyl acetate in pet ether) gave tert-butyl 3-(6-bromo-1,3-benzoxazol-2-yl)azetidine-1-carboxylate (4, 4 g, 11.13 mmol, 18% yield) as an off white solid. LC-MS (ES⁺): m/z 353.21 [M+H]⁺.

Step-3:

To a stirred solution of tert-butyl 3-(6-bromo-1,3-benzoxazol-2-yl)azetidine-1-carboxylate (4, 1.7 g, 4.81 mmol), bis(pinacolato)diboron (1.34 g, 5.29 mmol) and potassium acetate (1.18 g, 12.03 mmol, 752.15 μL) in 1,4 dioxane (20 mL) was purged with nitrogen for 5 minutes and added Pd(dppf)Cl₂ (393.05 mg, 481.30 μmol). The resulting mixture was stirred for 16 hr at 85° C. The reaction mixture was cooled to ambient temperature poured into water (50 mL) and extracted with ethyl acetate (3×50 mL). The combined organic extracts were washed with water and brine, dried over anhydrous sodium sulphate, filtered and evaporated to dryness. The resulting crude mixture was purified by column chromatography (silica gel, 0 to 20% ethyl acetate in pet ether) to give tert-butyl 3-[6-(4,4,5,5-tetramethyl-1,3,2-dioxaborolan-2-yl)-1,3-benzoxazol-2-yl]azetidine-1-carboxylate (5, 1.9 g, 4.46 mmol, 93% yield) as an off white solid. LC-MS (ES⁺): m/z 400.28 [M+H]⁺.

### Example 69

### Synthesis of 3-[2-(azetidin-3-yl)-1,3-benzoxazol-6-yl]-6-benzyl-5,8-dihydro-1H-pyrazolo[4,3-g]quinazolin-7-one

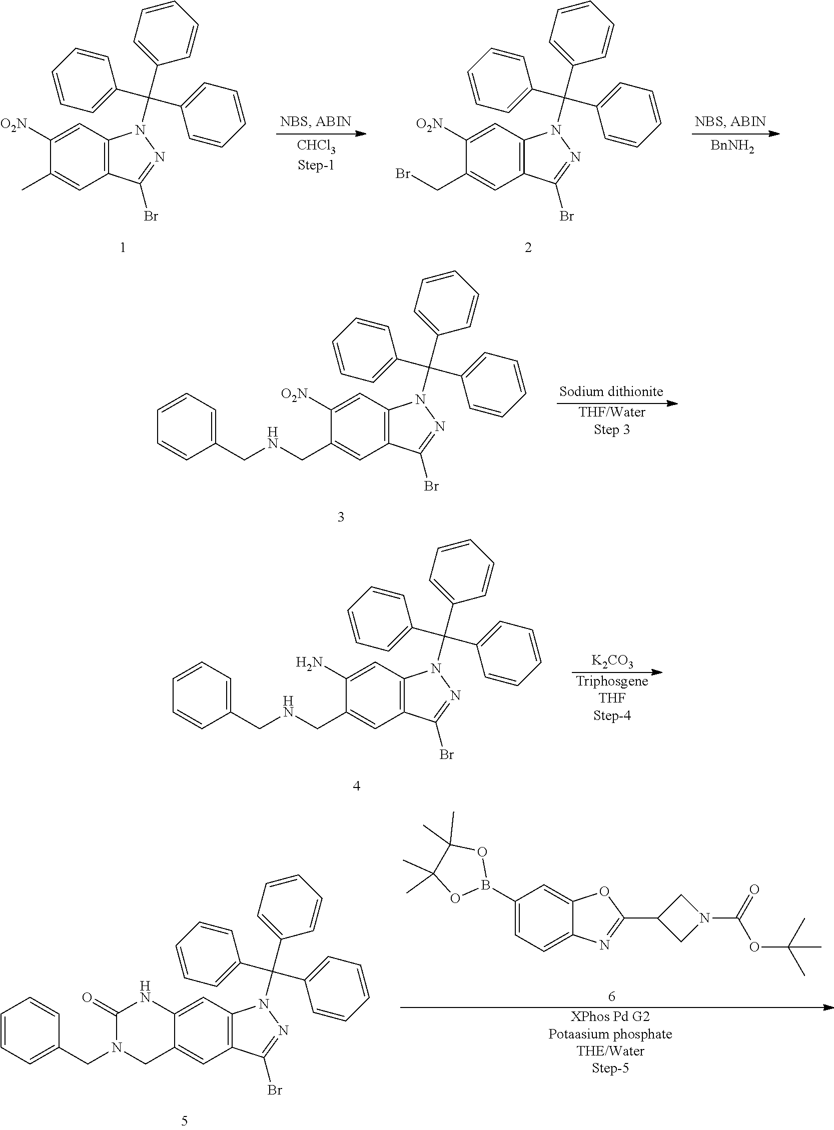

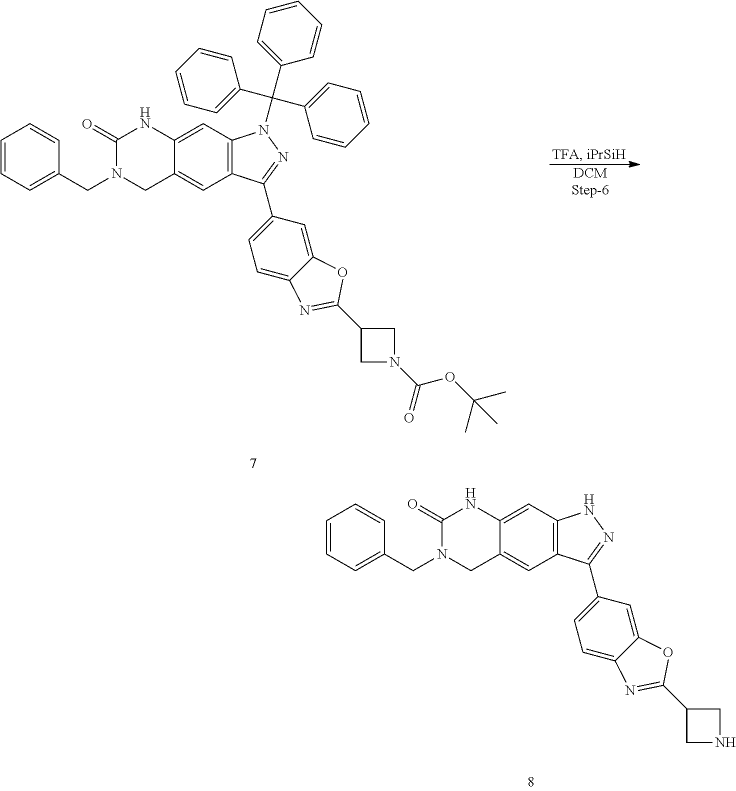

Step-1:

To a stirred solution of 3-bromo-5-methyl-6-nitro-1-trityl-indazole (1, 10.85 g, 21.77 mmol) in chloroform (100 mL) in a sealed tube was added NBS (11.62 g, 65.31 mmol, 5.54 mL) and azobisisobutyronitrile (357.50 mg, 2.18 mmol). The resulting mixture was stirred for 16 hr at 90° C. The reaction mixture was cooled to ambient temperature, diluted with water (100 mL) and extracted with DCM (3×150 mL). The combined organics were washed with water and brine, dried over anhydrous sodium sulphate, filtered and concentrated in-vacuo to give 3-bromo-5-(bromomethyl)-6-nitro-1-trityl-indazole (2, 12.55 g, 7.81 mmol, 36% yield) as a dark brown gummy solid. Used without further purification.

Step-2:

To a stirred solution of 3-bromo-5-(bromomethyl)-6-nitro-1-trityl-indazole (2, 12.55 g, 21.74 mmol) in DMF (100 mL), in a sealed tube, was added DIPEA (14.05 g, 108.70 mmol, 18.93 mL) and phenylmethanamine (3.49 g, 32.61 mmol) under a nitrogen atmosphere and the resulting mixture was heated to 90° C. for 16 hr. The reaction mixture was cooled to room temperature and diluted with water (100 ml) and then extracted with ethyl acetate (3×150 ml). The combined organics were washed with water, brine and dried over anhydrous Na₂SO₄, filtered and concentrated in-vacuo. Purification by silica gel column chromatography, eluting with 10% to 50% ethyl acetate in petroleum ether as eluent, gave N-[(3-bromo-6-nitro-1-trityl-indazol-5-yl)methyl]-1-phenyl-methanamine (3, 4.71 g, 7.61 mmol, 35% yield) as a pale yellow solid. LC-MS (ES⁺): m/z 603.2 [M]⁺. and 605.2 [M+2]⁺.

Step-3:

A 250 mL single-neck round bottomed flask was charged with a well-stirred solution of N-[(3-bromo-6-nitro-1-trityl-indazol-5-yl)methyl]-1-phenyl-methanamine (3, 8.34 g, 13.82 mmol) in THF (30 mL) and water (60 mL), to which was added sodium dithionite (12.03 g, 69.10 mmol) under a nitrogen atmosphere. The reaction mixture was stirred at room temperature for 3 hours. The reaction mixture was diluted with water (400 mL) and was stirred for 15 minutes. The solid formed was filtered and dried in-vacuo to yield 5-[(benzylamino)methyl]-3-bromo-1-trityl-indazol-6-amine (4, 8.5 g, 12.70 mmol, 92% yield) as a yellow solid. LC-MS (ES⁺): m/z 574.8 [M+H]⁺.

Step-4:

A 250 mL single-neck round bottomed flask was charged with well-stirred solution of 5-[(benzylamino)methyl]-3-bromo-1-trityl-indazol-6-amine (4, 3.5 g, 6.10 mmol) in THE (35 mL), to which was added potassium carbonate—granular (1.69 g, 12.21 mmol, 736.61 μL) and triphosgene (3.62 g, 12.21 mmol) in aliquots under a nitrogen atmosphere. The resulting mixture was stirred at room temperature for 3 hours. The reaction mixture was quenched by drop-wise addition of the reaction mixture into ice-cold water (400 mL), stirred for an hour and the resulting solid was filtered and dried in-vacuo to yield the crude 6-benzyl-3-bromo-1-trityl-5,8-dihydropyrazolo[4,3-g]quinazolin-7-one (5, 3.1 g, 3.94 mmol, 65% yield) as a cream solid. ¹H-NMR (400 MHz, DMSO-d6): δ 9.35 (s, 1H), 7.35-7.17 (m, 21H), 6.13 (s, 1H), 4.49 (s, 2H), 4.34 (s, 2H).

Step-5:

A 50 mL seal tube was charged with a well-stirred solution of 6-benzyl-3-bromo-1-trityl-5,8-dihydropyrazolo[4,3-g]quinazolin-7-one (5, 600 mg, 1.00 mmol) and tert-butyl 3-[6-(4,4,5,5-tetramethyl-1,3,2-dioxaborolan-2-yl)-1,3-benzoxazol-2-yl]azetidine-1-carboxylate (6, 600 mg, 1.50 mmol) in THE (11 mL) and water (4 mL). The reaction mixture was purged with nitrogen and potassium phosphate tribasic anhydrous (636.36 mg, 3.00 mmol) and XPhos Pd G2 (23.59 mg, 29.98 μmol) were added. The resulting mixture was heated to 80° C. under a nitrogen atmosphere for a period of 3 hours. The reaction mixture was cooled to room temperature, diluted with water (30 mL) and ethyl acetate (30 mL). The resulting mixture was filtered through a celite bed and the filtrate was extracted with ethyl acetate (2×40 mL). The combined organics were washed with brine, dried over anhydrous Na₂SO₄, filtered and concentrated in-vacuo to yield the crude which was purified by passing through silica gel column chromatography eluting with 0-100% ethyl acetate in petroleum ether, with the product eluting at 65% EA in petroleum ether to yield tert-butyl 3-[6-(6-benzyl-7-oxo-1-trityl-5,8-dihydropyrazolo[4,3-g]quinazolin-3-yl)-1,3-benzoxazol-2-yl]azetidine-1-carboxylate (7, 500 mg, 529.64 μmol, 53% yield) as a yellow solid. ¹H-NMR (400 MHz, DMSO-d6): δ 9.30 (s, 1H), 8.11 (d, J=0.80 Hz, 1H), 7.91 (s, 1H), 7.86-7.83 (m, 1H), 7.77 (d, J=8.40 Hz, 1H), 7.37-7.23 (m, 20H), 6.17 (s, 1H), 4.52 (s, 2H), 4.38 (s, 2H), 4.31-4.29 (m, 2H), 4.17-4.15 (m, 3H), 1.42 (s, 9H).

Step-6:

To a stirred solution of tert-butyl 3-[6-(6-benzyl-7-oxo-1-trityl-5,8-dihydropyrazolo[4,3-g]quinazolin-3-yl)-1,3-benzoxazol-2-yl]azetidine-1-carboxylate (7, 200 mg, 252.23 μmol) in DCM (3 mL) was added trifluoroacetic acid (2.96 g, 25.96 mmol, 2.00 mL) followed by triisopropylsilane (773.00 mg, 4.88 mmol, 1 mL). The reaction mixture was stirred at room temperature for 2 hours. The reaction mixture was concentrated to give crude material that was washed with pet ether (to remove trityl by-product) and dried to give 3-[2-(azetidin-3-yl)-1,3-benzoxazol-6-yl]-6-benzyl-5,8-dihydro-1H-pyrazolo[4,3-g]quinazolin-7-one (8, 180 mg, 286.97 μmol, 76% yield) as pale brown gummy oil, which was immediately used without further purification in the next step. LC-MS (ES⁺): m/z 467 [M+18]⁺.

### Example 70

### Synthesis of 3-[6-[1-[2-[3-[6-(6-benzyl-7-oxo-5,8-dihydro-1H-pyrazolo[4,3-g]quinazolin-3-yl)-1,3-benzoxazol-2-yl]azetidin-1-yl]-2-oxo-ethyl]-4-piperidyl]-2-oxo-benzo[cd]indol-1-yl]piperidine-2,6-dione (Compound 100)

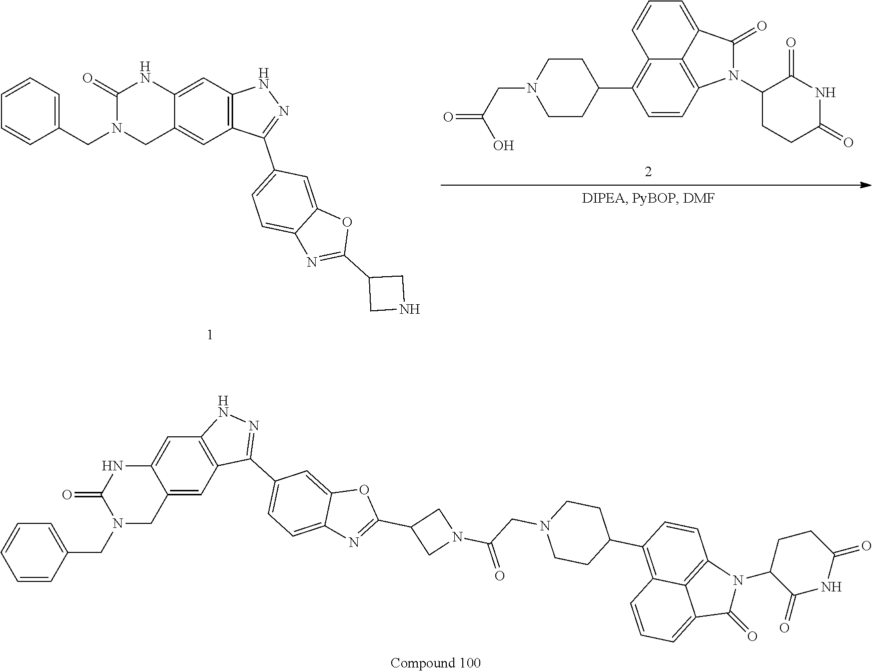 [SMILES: O=C1Nc2cc3[nH]nc(-c4ccc5nc(C6CNC6)oc5c4)c3cc2CN1Cc1ccccc1 ; O=C(O)CN1CCC(c2ccc3c4c(cccc24)C(=O)N3C2CCC(=O)NC2=O)CC1 ; O=C1CCC(N2C(=O)c3cccc4c(C5CCN(CC(=O)N6CC(c7nc8ccc(-c9n[nH]c%10cc%11c(cc9%10)CN(Cc9ccccc9)C(=O)N%11)cc8o7)C6)CC5)ccc2c34)C(=O)N1]

To a stirred solution of 3-[2-(azetidin-3-yl)-1,3-benzoxazol-6-yl]-6-benzyl-5,8-dihydro-1H-pyrazolo[4,3-g]quinazolin-7-one (1, 50 mg, 110.99 μmol), 2-[4-[1-(2,6-dioxo-3-piperidyl)-2-oxo-benzo[cd]indol-6-yl]-1-piperidyl]acetic acid (2, 51.45 mg, 122.09 μmol) and DIPEA (71.72 mg, 554.95 μmol, 96.66 μL) in N,N-Dimethyl formamide (3 mL) was added PyBOP (115.52 mg, 221.98 μmol) and the reaction mixture was stirred at room temperature for 16 h. Cold water was added to the reaction mixture and then extracted with dichloromethane. Then combined organic extracts were dried over anhydrous sodium sulphate and then concentrated. The crude material was purified by prep HPLC to yield the product 3-[6-[1-[2-[3-[6-(6-benzyl-7-oxo-5,8-dihydro-1H-pyrazolo[4,3-g]quinazolin-3-yl)-1,3-benzoxazol-2-yl]azetidin-1-yl]-2-oxo-ethyl]-4-piperidyl]-2-oxo-benzo[cd]indol-1-yl]piperidine-2,6-dione (12 mg, 13.08 μmol, 12% yield) as light yellow solid. LC-MS (ES⁺): m/z 854 [M+H]⁺. ¹H-NMR (400 MHz, DMSO-d6): δ 12.98 (s, 1H), 11.14 (s, 1H), 9.59 (s, 1H), 8.46-8.44 (m, 1H), 8.22 (d, J=1.20 Hz, 1H), 8.12 (d, J=7.20 Hz, 1H), 8.00 (dd, J=1.60, 8.40 Hz, 1H), 7.91-7.81 (m, 3H), 7.40-7.25 (m, 6H), 7.12-6.95 (m, 1H), 6.96 (s, 1H), 5.48-5.41 (m, 1H), 4.81-4.73 (m, 1H), 4.61-4.59 (m, 3H), 4.44 (s, 2H), 4.40-4.31 (m, 1H), 4.30-4.21 (m, 2H), 3.04-2.96 (m, 5H), 2.78-2.61 (m, 2H), 2.11-2.08 (m, 2H), 1.92-1.26 (m, 3H), 1.72-1.68 (m, 3H).

### Example 71

### Synthesis of 3-[6-[1-[4-[3-[6-(6-benzyl-7-oxo-5,8-dihydro-1H-pyrazolo[4,3-g]quinazolin-3-yl)-1,3-benzoxazol-2-yl]azetidin-1-yl]-4-oxo-butyl]-4-piperidyl]-2-oxo-benzo[cd]indol-1-yl]piperidine-2,6-dione (Compound 101)

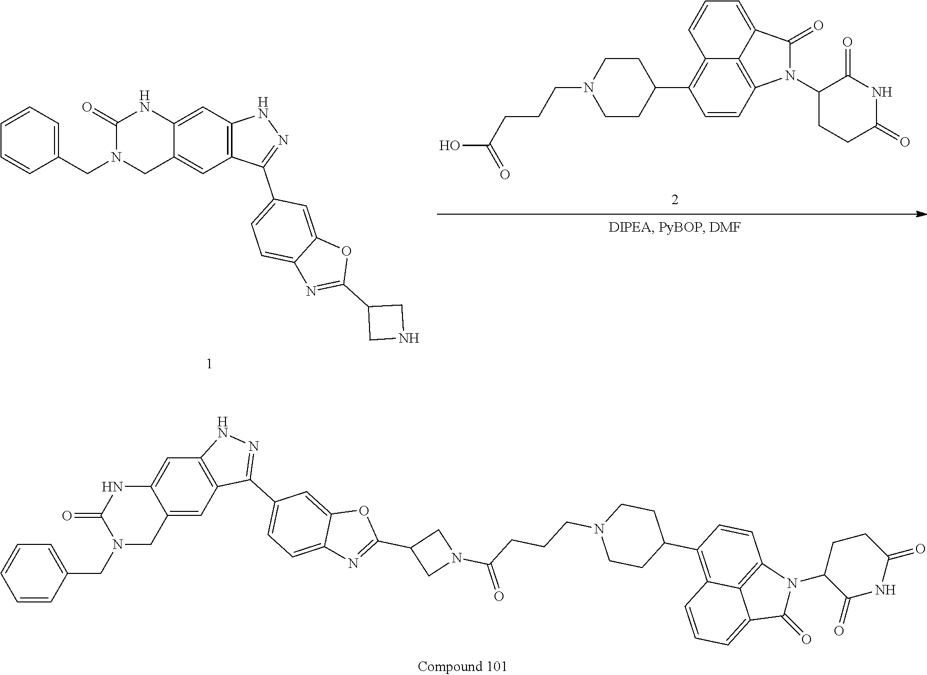 [SMILES: O=C1Nc2cc3[nH]nc(-c4ccc5nc(C6CNC6)oc5c4)c3cc2CN1Cc1ccccc1 ; O=C(O)CCCN1CCC(c2ccc3c4c(cccc24)C(=O)N3C2CCC(=O)NC2=O)CC1 ; O=C1CCC(N2C(=O)c3cccc4c(C5CCN(CCCC(=O)N6CC(c7nc8ccc(-c9n[nH]c%10cc%11c(cc9%10)CN(Cc9ccccc9)C(=O)N%11)cc8o7)C6)CC5)ccc2c34)C(=O)N1]

To a stirred solution of 3-[2-(azetidin-3-yl)-1,3-benzoxazol-6-yl]-6-benzyl-5,8-dihydro-1H-pyrazolo[4,3-g]quinazolin-7-one (1, 50 mg, 110.99 μmol), 4-[4-[1-(2,6-dioxo-3-piperidyl)-2-oxo-benzo[cd]indol-6-yl]-1-piperidyl]butanoic acid (2, 54.88 mg, 122.09 μmol) and DIPEA (71.72 mg, 554.95 μmol, 96.66 μL) in N,N-dimethyl formamide (3 mL) was added PyBOP (115.52 mg, 221.98 μmol) and the reaction mixture was stirred at room temperature for 16 h. Cold water was added to the reaction mixture and then extracted with dichloromethane. The combined organic extracts were dried over anhydrous sodium sulphate and concentrated. The crude material was purified by prep HPLC to yield the product 3-[6-[1-[4-[3-[6-(6-benzyl-7-oxo-5,8-dihydro-1H-pyrazolo[4,3-g]quinazolin-3-yl)-1,3-benzoxazol-2-yl]azetidin-1-yl]-4-oxo-butyl]-4-piperidyl]-2-oxo-benzo[cd]indol-1-yl]piperidine-2,6-dione (12 mg, 13.06 μmol, 12% yield) as light yellow solid. LC-MS (ES⁺): m/z 882 [M+H]⁺. ¹H-NMR (400 MHz, DMSO-d6): δ 12.96 (s, 1H), 11.12 (s, 1H), 9.59 (s, 1H), 8.38 (d, J=8.00 Hz, 1H), 8.16 (d, J=4.40 Hz, 1H), 8.06 (d, J=6.80 Hz, 1H), 7.96 (dd, J=1.20, 8.20 Hz, 1H), 7.87-7.77 (m, 3H), 7.39-7.30 (m, 6H), 7.11-6.96 (m, 1H), 6.94 (s, 1H), 5.43-5.40 (m, 1H), 4.65-4.59 (m, 3H), 4.53-4.50 (m, 1H), 4.42 (s, 2H), 4.35-4.22 (m, 3H), 3.02-3.00 (m, 5H), 2.16-2.08 (m, 7H), 1.91-1.80 (m, 7H).

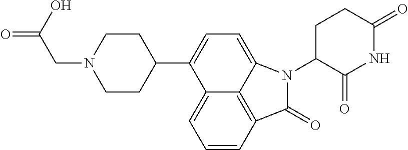 [SMILES: O=C(O)CN1CCC(c2ccc3c4c(cccc24)C(=O)N3C2CCC(=O)NC2=O)CC1]

Compound 2-[4-[1-(2,6-dioxo-3-piperidyl)-2-oxo-benzo[cd]indol-6-yl]-1-piperidyl]acetic acid was prepared according to the method described on page 203-204 of WO2021127586A1.

### Example 72

### Synthesis of 2-[4-[1-(2,6-dioxo-3-piperidyl)-2-oxo-benzo[cd]indol-5-yl]-1-piperidyl]acetic acid

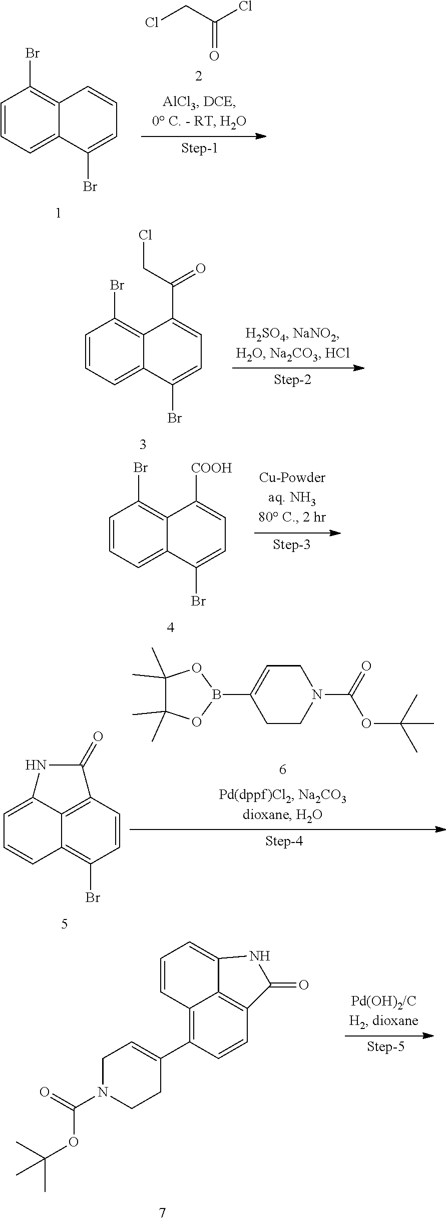

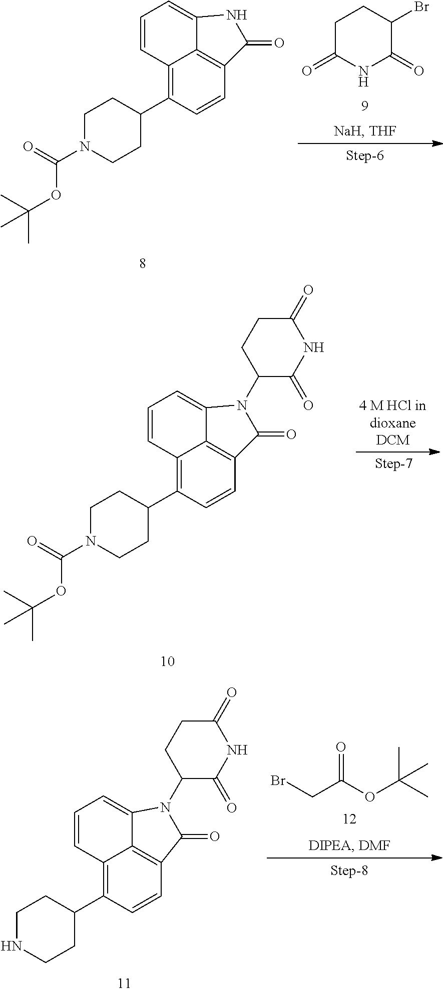

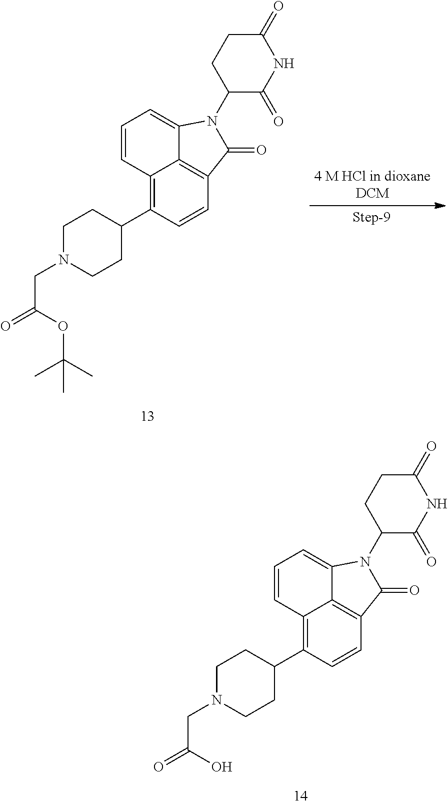 [SMILES: CC(C)(C)OC(=O)CN1CCC(c2ccc3c4c(cccc24)N(C2CCC(=O)NC2=O)C3=O)CC1 ; O=C(O)CN1CCC(c2ccc3c4c(cccc24)N(C2CCC(=O)NC2=O)C3=O)CC1]

Step-1:

A stirred solution of 1,5-dibromonaphthalene (1, 162 g, 566.51 mmol) in DCE (2000 mL) was cooled to 0° C. and 2-chloroacetyl chloride (2, 83.18 g, 736.46 mmol, 58.57 mL) was added dropwise. The resultant solution was stirred at 0° C. for 15 minutes followed by portion-wise addition of anhydrous aluminum chloride (98.20 g, 736.46 mmol, 40.25 mL). The resultant reaction mixture was then slowly warmed to room temperature and stirred for 16 hours. After completion (monitored by TLC) the reaction mixture was poured into ice cold water and extracted with DCM (twice). The combined organic extract was further washed with water and brine, dried over sodium sulfate, filtered and concentrated under reduced pressure. The crude thus obtained was purified by column chromatography (100-200 Silica; Gradient: 0-5% EtOAc in hexane) to afford 2-chloro-1-(4,8-dibromo-1-naphthyl)ethanone (3, 150 g, 390 mmol, 69% yield) as an off-white solid. ¹H NMR (400 MHz, DMSO d6) δ 8.36 (dd, J=8.48, 0.72 Hz, 1H), 8.11-8.07 (m, 2H), 7.69 (t, J=8.04 Hz, 1H), 7.59 (d, J=7.8 Hz, 1H), 5.05 (s, 2H);

Step-2:

To a stirred solution of 2-chloro-1-(4,8-dibromo-1-naphthyl)ethanone (3, 151 g, 416.62 mmol) in sulfuric acid (1.8 L) was added sodium nitrite (30.27 g, 438.75 mmol) at room temperature and the resultant reaction mixture was stirred at 65° C. for 45 minutes. After completion of the reaction, the reaction mixture was poured into cold water (2 litres) and the resulting solid was filtered off. The solid thus obtained was added to a 10% sodium carbonate solution (4 lit) and stirred for 30 minutes at room temperature. The mixture was filtered; the filtrate was cautiously acidified with concentrated HCl under vigorous stirring and filtered again to remove insoluble impurity. The filtrate (aqueous) was then extracted with ethyl acetate (twice). The combined organic extract was further washed with brine, dried over sodium sulfate and concentrated under reduced pressure to afford 4,8-dibromonaphthalene-1-carboxylic acid (4, 110 g, 299 mmol, 72% yield) as alight brown solid. ¹H NMR (400 MHz, DMSO d6) δ 13.48 (br s, 1H), 8.33 (d, J=8.36 Hz, 1H), 8.09 (d, J=7.4 Hz, 1H), 8.01 (d, J=7.72 Hz, 1H), 7.65 (t, J=8.0 Hz, 1H), 7.59 (d, J=7.72 Hz, 1H). LC-MS (ES⁻): m/z 328.90 [M−H]⁻.

Step-3:

To a stirred suspension of 4,8-dibromonaphthalene-1-carboxylic acid (4, 65 g, 196.99 mmol) in aqueous ammonia (700 mL) was added copper powder (3.25 g, 51.22 mmol) and the resultant reaction mixture was stirred at 80° C. for 2 hours. After completion (monitored by TLC) the reaction mixture was poured into ice-cooled water and was slowly acidified with concentrated HCl (pH-2) with vigorous stirring. The resulting yellow precipitate was filtered dried under reduced pressure to afford 5-bromo-1H-benzo[cd]indol-2-one (5, 39 g, 151.68 mmol, 77% yield) as brown solid. ¹H NMR (400 MHz, DMSO d6) δ 10.88 (s, 1H), 8.05 (d, J=7.44 Hz, 1H), 7.88 (d, J=7.4 Hz, 1H), 7.61 (t, J=7.8 Hz, 1H), 7.53 (d, J=8.56 Hz, 1H), 7.04 (d, J=7.0 Hz, 1H). LC-MS (ES⁺): m/z 248.2 [M+H]⁺.

Step-4:

To a solution of 5-bromo-1H-benzo[cd]indol-2-one (5, 3 g, 12.09 mmol) in dioxane (30 mL) and water (10 mL) were added tert-butyl 4-(4,4,5,5-tetramethyl-1,3,2-dioxaborolan-2-yl)-3,6-dihydro-2H-pyridine-1-carboxylate (6, 5.61 g, 18.14 mmol) and sodium carbonate (3.85 g, 36.28 mmol) at room temperature and the reaction mixture was purged with nitrogen gas for 15 minutes. To this solution was added cyclopentyl(diphenyl)phosphane;dichloromethane;dichloropalladium;iron (987.57 mg, 1.21 mmol) at room temperature and the reaction mixture was purged with nitrogen gas for another 5 minutes. The reaction mixture was stirred at 90° C. for 10 hours while monitoring by LCMS. Upon completion of the reaction, the reaction mixture was allowed to cool at room temperature, poured onto water (100 mL) and extracted with ethyl acetate (2×250 mL) and washed with brine (100 ml) and dried over anhydrous sodium sulfate and concentrated on rotavapor to get the crude which was purified by Biotage® Isolera flash column chromatography (100-200 mesh 50 g silica gel, 0-100% ethyl acetate in hexane) to afford tert-butyl 4-(2-oxo-1H-benzo[cd]indol-5-yl)-3,6-dihydro-2H-pyridine-1-carboxylate (7, 2.5 g, 6.87 mmol, 57% yield) as a pale yellow solid. LC-MS (ES⁺): m/z 351.2 [M+H]⁺.

Step-5:

To a stirred solution of tert-butyl 4-(2-oxo-1H-benzo[cd]indol-5-yl)-3,6-dihydro-2H-pyridine-1-carboxylate (7, 2.5 g, 7.13 mmol) in 1,4-dioxane (25 mL) was added palladium hydroxide on carbon, 20 wt. % 50% water (1.2 g, 8.54 mmol) under nitrogen atmosphere. The resulting reaction mixture was stirred under hydrogen atmosphere for 4 hours. Upon completion of the reaction, the reaction mixture was filtered through celite bed, washed with ethyl acetate (100 mL) and concentrated under vacuum. The crude compound was purified by column chromatography using 100-200 mesh silica gel and eluted with 0-25% ethyl acetate in pet ether to afford tert-butyl 4-(2-oxo-1H-benzo[cd]indol-5-yl)piperidine-1-carboxylate (8, 1.7 g, 4.58 mmol, 64% yield) as a pale yellow solid. LC-MS (ES⁺): m/z 297.0 [M-56+H]⁺:

Step-6:

In a 250 mL three neck round bottom flask, to a solution of tert-butyl 4-(2-oxo-1H-benzo[cd]indol-5-yl)piperidine-1-carboxylate (8, 0.8 g, 2.27 mmol) in THE (100 mL) was added sodium hydride (60% dispersion in mineral oil) (720.01 mg, 18.79 mmol) at 0° C. and the mixture was stirred at room temperature for 60 minutes. This was followed by the addition 3-bromopiperidine-2,6-dione (9, 1.31 g, 6.81 mmol) in THE (10 mL) at 0° C. The resulting reaction mixture was heated to 60° C. for 4 hours. Upon completion of the reaction, the reaction mixture was quenched with saturated ammonium chloride (50 mL) at 0° C. and extracted with ethyl acetate (2×100 mL), dried over anhydrous sodium sulfate, and concentrated in vacuo. The crude product was purified by column chromatography (silica gel, 0-40% ethyl acetate in pet ether) to afford tert-butyl 4-[1-(2,6-dioxo-3-piperidyl)-2-oxo-benzo[cd]indol-5-yl]piperidine-1-carboxylate (10, 0.5 g, 855.40 μmol, 38% yield) as a pale yellow solid. LC-MS (ES⁺): m/z 464.2 [M+H]⁺.

Step-7:

To a stirred solution of tert-butyl 4-[1-(2,6-dioxo-3-piperidyl)-2-oxo-benzo[cd]indol-5-yl]piperidine-1-carboxylate (10, 0.85 g, 1.83 mmol) in DCM (30 mL) was added 4.0M hydrogen chloride solution in dioxane (12.95 g, 355.25 mmol, 16.19 mL). The resulting reaction mixture was stirred at room temperature for 4 hours while monitoring by TLC and UPLC. The reaction mixture was concentrated under vacuum and washed with pet ether (50 mL) to obtain 3-[2-oxo-5-(4-piperidyl)benzo[cd]indol-1-yl]piperidine-2,6-dione hydrochloride (11, 0.68 g, 1.64 mmol, 89% yield) as an off-white solid. The crude compound was taken directly next step without further purification. LC-MS (ES⁺): m/z 364.2 [M+H]⁺.

Step-8:

To a solution of 3-[2-oxo-5-(4-piperidyl)benzo[cd]indol-1-yl]piperidine-2,6-dione hydrochloride (11, 250 mg, 625.20 μmol) in DMF (5 mL) were added DIPEA (808.01 mg, 6.25 mmol, 1.09 mL) and tert-butyl 2-bromoacetate (12, 182.92 mg, 937.81 μmol, 137.54 μL) at room temperature. The reaction mixture was stirred at this temperature for 4 hours and the progress of the reaction mixture was monitored by UPLC and TLC. The reaction mixture was poured into ice cold water (50 mL) and extracted with ethyl acetate (2×50 mL), dried over anhydrous sodium sulfate, filtered and concentrated in vacuo. The crude product was washed with pet ether (50 mL) to afford tert-butyl 2-[4-[1-(2,6-dioxo-3-piperidyl)-2-oxo-benzo[cd]indol-5-yl]-1-piperidyl]acetate (13, 230 mg, 446.61 μmol, 71% yield) as an off-white solid. LC-MS (ES⁺): m/z 478.4 [M+H]⁺.

Step-9:

To a stirred solution of tert-butyl 2-[4-[1-(2,6-dioxo-3-piperidyl)-2-oxo-benzo[cd]indol-5-yl]-1-piperidyl]acetate (13, 230 mg, 481.62 μmol) in DCM (5 mL) added 4.0 M hydrogen chloride solution in dioxane (5.26 g, 144.19 mmol, 6.57 mL) at room temperature. The resulting reaction mixture was stirred a this temperature for 40 hours and the reaction progress was monitored by TLC and UPLC. The reaction mixture was concentrated under reduced pressure and the crude product was washed with pet ether (50 mL) and concentrated to afford 2-[4-[1-(2,6-dioxo-3-piperidyl)-2-oxo-benzo[cd]indol-5-yl]-1-piperidyl]acetic acid hydrochloride (14, 180 mg, 371.08 μmol, 77% yield) as an off-white solid. LC-MS (ES⁺): m/z 422.2 [M+H]⁺.

### Example 73

### Synthesis of N-[(3-fluorophenyl)methyl]-1-[1-[4-[2-(4-piperidyl)ethynyl]-1-naphthyl]ethyl]piperidine-4-carboxamide hydrochloride

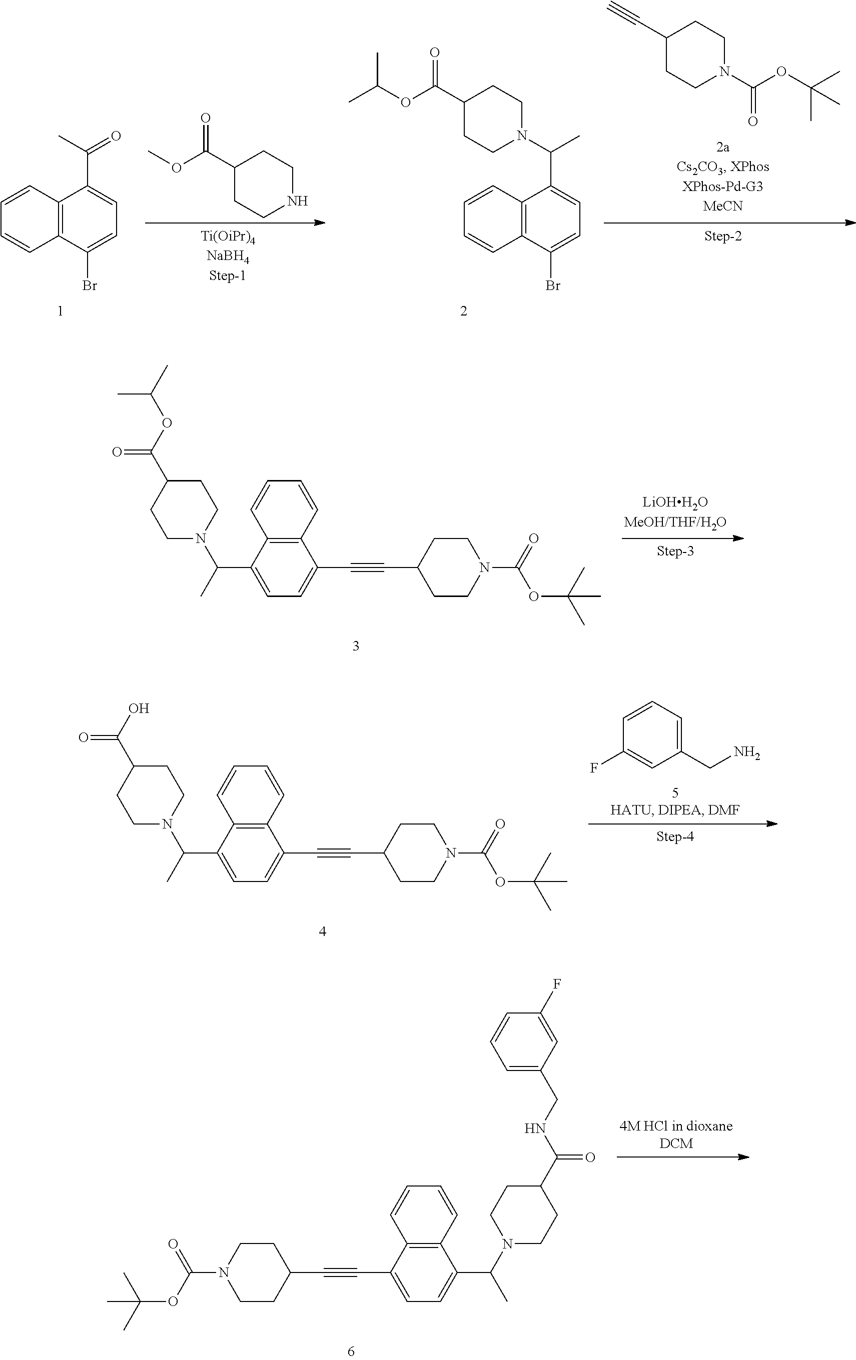

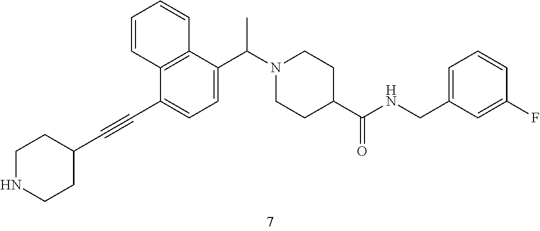 [SMILES: CC(c1ccc(C#CC2CCNCC2)c2ccccc12)N1CCC(C(=O)NCc2cccc(F)c2)CC1]

Step-1:

To a 250 mL round bottom flask containing a well-stirred solution of a mixture of 1-(4-bromo-1-naphthyl)ethanone (1, 1, 5.0 g, 19.07 mmol) in neat anhydrous titanium(IV) isopropoxide (50.31 mL) were added methyl piperidine-4-carboxylate (2, 4.10 g, 28.60 mmol, 3.86 mL) at ambient temperature. The reaction mixture was stirred at 80° C. for 6 h. The reaction mixture was cooled to 0° C. and Sodium Borohydride (3.60 g, 95.14 mmol, 3.35 mL) was added to the reaction mixture. The reaction mixture was stirred at 30° C. for 3 h. After completion of the reaction, reaction mixture was cooled to 0° C. and reaction mass was diluted with water, and solid precipitated out was filtered. Filtrate was extracted with ethyl acetate (2×150 mL). Organic phases were combined and washed with brine (100 mL). Combined organic phases were dried (anhydrous Na₂SO₄), filtered and the filtrate was concentrated under reduced pressure to get a crude residue, which was purified by flash silica-gel (60-120 mesh) column with 0-100% ethyl acetate in petroleum ether to afford isopropyl 1-[1-(4-bromo-1-naphthyl)ethyl]piperidine-4-carboxylate (3, 3.5 g, 8.14 mmol, 43% yield) as a colorless thick liquid. LC-MS (ES⁺): m/z 404.2 [M+H]⁺.

Step-2:

To a 250 mL sealed tube containing a well stirred solution of isopropyl 1-[1-(4-bromo-1-naphthyl)ethyl]piperidine-4-carboxylate (3, 5 g, 12.37 mmol), tert-butyl 4-ethynylpiperidine-1-carboxylate (4, 3.36 g, 16.08 mmol) in anhydrous acetonitrile (60 mL) was added cesium carbonate (10.07 g, 30.91 mmol) at room temperature. The reaction mixture was purged with nitrogen gas for 10 minutes before XPhos (589.50 mg, 1.24 mmol) and XPhos-Pd-G3 (524.00 mg, 618.29 mol) were added. The reaction was stirred at 90° C. for 5 hours. Upon completion of the reaction, the reaction mixture was filtered through a pad of celite, washed with ethyl acetate (500 mL) and the filtrate was concentrated under reduced pressure to yield the crude compound, which was purified by flash column chromatography (230-400 mesh silica gel, 40% ethyl acetate in petroleum ether) to afford tert-butyl 4-[2-[4-[1-(4-isopropoxycarbonyl-1-piperidyl)ethyl]-1-naphthyl]ethynyl]piperidine-1-carboxylate (5, 4 g, 7.36 mmol, 60% yield) as a light brown gummy solid. LC-MS (ES⁺): m/z 533.2 [M+H]⁺.

Step-3:

To a 250 mL single neck round bottom flask containing a stirred solution tert-butyl 4-[2-[4-[1-(4-isopropoxycarbonyl-1-piperidyl)ethyl]-1-naphthyl]ethynyl]piperidine-1-carboxylate (5, 4.08 g, 7.51 mmol) in methanol (40 mL) and THE (40 mL)and water (20 mL) was added lithium hydroxide monohydrate, 98% (3.15 g, 75.09 mmol, 2.09 mL) at ambient temperature and the resulting mixture was stirred for 3 hours. Upon completion of the reaction, the volatiles were evaporated under vacuum to yield the crude product, which was acidified with 10% citric acid solution to pH=4 and extracted with 10% MeOH in DCM (2×400 mL). The combined organic layers were dried over Na₂SO₄ and concentrated under reduced pressure to afford 1-[1-[4-[2-(1-tert-butoxycarbonyl-4-piperidyl)ethynyl]-1-naphthyl]ethyl]piperidine-4-carboxylic acid (6, 3.67 g, 7.41 mmol, 99% yield) as an brown color solid. LC-MS (ES⁺): m/z 491.2 [M+H]⁺.

Step-4:

To a 100 mL round bottom flask containing a well-stirred solution of 1-[1-[4-[2-(1-tert-butoxycarbonyl-4-piperidyl)ethynyl]-1-naphthyl]ethyl]piperidine-4-carboxylic acid (6, 4 g, 7.51 mmol) and (3-fluorophenyl)methanamine (7, 940.19 mg, 7.51 mmol, 857.05 μL) in anhydrous DMF (40 mL) was added N,N-diisopropylethylamine (4.85 g, 37.56 mmol, 6.54 mL) at room temperature under nitrogen atmosphere. After 5 minutes, HATU (4.28 g, 11.27 mmol) was added, and the resulting mixture was stirred at room temperature for 3 hours. Upon completion of the reaction, the reaction mixture was quenched with water (100 mL), extracted with ethyl acetate (3×150 mL), dried over sodium sulfate and concentrated under reduced pressure to give the crude product, which was purified by column chromatography (100 g silica gel column, 0-100% ethyl acetate in petroleum ether) to afford tert-butyl 4-[2-[4-[1-[4-[(3-fluorophenyl)methylcarbamoyl]-1-piperidyl]ethyl]-1-naphthyl]ethynyl]piperidine-1-carboxylate (8, 3.5 g, 5.57 mmol, 74% yield) as a brown color solid. LC-MS (ES⁺): m/z 598.2 [M+H]⁺.

Step-5:

To a 100 mL single-neck round-bottom flask containing a well stirred solution of tert-butyl 4-[2-[4-[1-[4-[(3-fluorophenyl)methylcarbamoyl]-1-piperidyl]ethyl]-1-naphthyl]ethynyl]piperidine-1-carboxylate (8, 3.5 g, 5.56 mmol) in anhydrous DCM (5 mL) was added 4 M hydrogen chloride in 1,4-dioxane (5.56 mmol) at 0° C. The contents were stirred at room temperature for 2 hours. After completion of the reaction, the solvent was removed to give the crude compound, which was dissolved with toluene, evaporated to dryness, and washed with MTBE to afford N-[(3-fluorophenyl)methyl]-1-[1-[4-[2-(4-piperidyl)ethynyl]-1-naphthyl]ethyl]piperidine-4-carboxamide hydrochloride (9, 3 g, 5.21 mmol, 94% yield) as a brown color solid. LC-MS (ES⁺): m/z 498.2 [M+H]⁺.

### Example 74

### Synthesis of N-[(3-fluorophenyl)methyl]-1-[1-[5-[2-(4-piperidyl)ethynyl]-1-naphthyl]ethyl]piperidine-4-carboxamide dihydrochloride

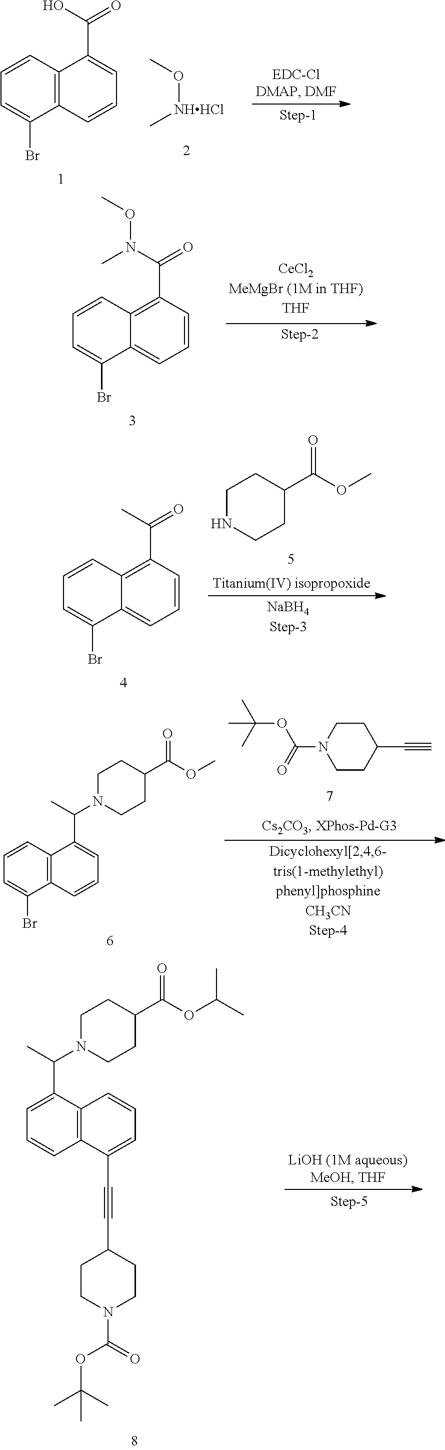

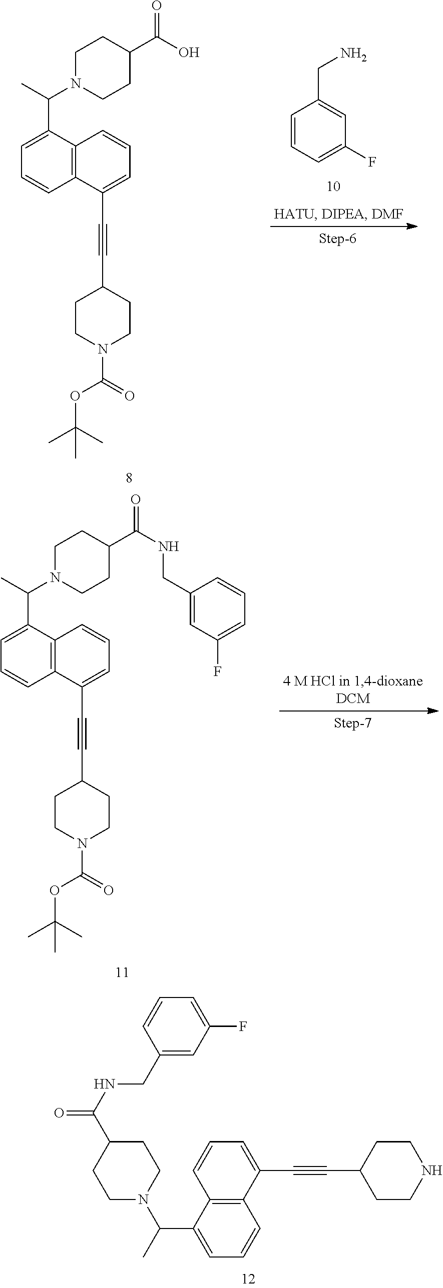 [SMILES: CC(c1cccc2c(C#CC3CCN(C(=O)OC(C)(C)C)CC3)cccc12)N1CCC(C(=O)O)CC1 ; NCc1cccc(F)c1 ; CC(c1cccc2c(C#CC3CCN(C(=O)OC(C)(C)C)CC3)cccc12)N1CCC(C(=O)NCc2cccc(F)c2)CC1 ; CC(c1cccc2c(C#CC3CCNCC3)cccc12)N1CCC(C(=O)NCc2cccc(F)c2)CC1]

Step-1:

To a 100 mL single-neck round-bottom flask containing a well-stirred solution of 5-bromonaphthalene-1-carboxylic acid (1, 8 g, 31.86 mmol) in anhydrous DMF (200 mL) were added N,N-dimethylpyridin-4-amine (11.68 g, 95.59 mmol) and 3-(ethyliminomethyleneamino)-N,N-dimethyl-propan-1-amine hydrochloride (12.22 g, 63.73 mmol) followed by N-methoxymethanamine hydrochloride (2, 12.43 g, 127.45 mmol) at ambient temperature under nitrogen atmosphere. The contents were stirred at ambient temperature for 16 hours. After completion of the reaction, the reaction mixture was poured into water (500 mL) and extracted with EtOAc (2×500 mL). The combined organic layers were washed with brine (300 mL), dried over anhydrous Na₂SO₄, and filtered. The filtrate was concentrated under reduced pressure to get the crude product, which was purified by column chromatography (100 g silica gel, 0-100% EtOAc/petroleumPetroleum ether) to afford 5-bromo-N-methoxy-N-methyl-naphthalene-1-carboxamide (3, 9 g, 26.35 mmol, 83% yield) as a thick colorless liquid. LC-MS (ES⁺): m/z 295.9 [M+H]⁺.

Step-2:

To a 500 mL 3-necked round-bottomed flask containing a well-stirred solution of 5-bromo-N-methoxy-N-methyl-naphthalene-1-carboxamide (3, 8.5 g, 24.88 mmol) in anhydrous THE (100 mL) was added anhydrous cerium (III) chloride (9.20 g, 37.32 mmol) at 0° C. The resulting reaction mixture was stirred for 1 hour at room temperature before it was cooled to 0° C. and added methyl magnesium bromide, 1 M solution in THE (149.28 mL. 149.28 mmol) dropwise. The resulting solution was stirred at room temperature 12 hours. Upon completion of the reaction, the reaction mixture was quenched slowly with saturated NH₄Cl solution (200 mL) under 0° C. The reaction mixture was filtered through a pad of celite, and the filter cake was washed with EtOAc and extracted with EtOAc (2×500 mL). The combined organic layers were washed with brine (150 mL), dried over anhydrous Na₂SO₄, and filtered. The filtrate was concentrated under reduced pressure to give the crude product, which was purified by column chromatography (100 g, silica gel, 0-100% EtOAc/Petroleum ether) to afford 1-(5-bromo-1-naphthyl)ethanone (4, 6.5 g, 24.74 mmol, 99% yield) as a thick colorless liquid. LCMS (ES⁻): m/z 249.2 [M−H]⁻.

Step-3:

In a 100 mL sealed tube, 1-(5-bromo-1-naphthyl)ethanone (4, 6 g, 22.84 mmol), methyl piperidine-4-carboxylate (5, 4.91 g, 34.26 mmol, 4.63 mL) and anhydrous titanium(IV) isopropoxide (60 mL) were mixed at ambient temperature. The reaction mixture was stirred at 80° C. for 6 hours. The reaction mixture was cooled to 0° C. and sodium borohydride (2.59 g, 68.52 mmol, 2.42 mL) was added. It was then stirred at 30° C. for 3 hours. After completion of the reaction, the reaction mixture was cooled to 0° C. and diluted with EtOAc, washed successively with saturated sodium bicarbonate solution The solid precipitation was filtered and the filtrate was extracted with EtOAc (2×150 mL). The combined organic phases were washed with brine (100 mL), dried over anhydrous Na₂SO₄, filtered and concentrated under reduced pressure to give the crude product, which was purified by flash column chromatography (230-400 mesh silica-gel neutralized with 10% triethylamine/petroleum ether, 0-100% EtOAc/petroleum ether) to afford isopropyl 1-[1-(5-bromo-1-naphthyl)ethyl]piperidine-4-carboxylate (6, 7 g, 14.97 mmol, 66% yield) as a colorless thick liquid. LC-MS (ES⁺): m/z 405.9 [M+H]⁺.

Step-4:

To a 50 mL sealed-tube containing a well-stirred solution of isopropyl 1-[1-(5-bromo-1-naphthyl)ethyl]piperidine-4-carboxylate (6, 1 g, 2.13 mmol) and tert-butyl 4-ethynylpiperidine-1-carboxylate (7, 578.67 mg, 2.77 mmol) in anhydrous acetonitrile (10 mL) was added cesium carbonate (2.08 g, 6.38 mmol) at ambient temperature under nitrogen atmosphere. The resulting mixture was degassed with nitrogen gas for 10 minutes. Subsequently, XPhos-Pd-G3 (90.02 mg, 106.35 μmol) and dicyclohexyl-[2-(2,4,6-triisopropylphenyl)phenyl]phosphane (101.39 mg, 212.69 μmol) were added and the resulting mixture was degassed with nitrogen gas for another 5 minutes before being heated at 90° C. for 4 hours. Upon consumption of the starting material, the reaction mixture was then cooled to ambient temperature and poured into water (100 mL) and EtOAc (100 mL). It was then filtered through a pad of celite, and the filter cake was washed with EtOAc (50 mL) and extracted with EtOAc (2×150 mL). The combined organic layers were washed with brine (150 mL), dried over anhydrous Na₂SO₄, and filtered. The filtrate was concentrated under reduced pressure to give the crude product, which was purified by Biotage® Isolera (230-400 mesh silica-gel with 0-100% ethyl acetate/petroleumPetroleum ether) to afford tert-butyl 4-[2-[5-[1-(4-isopropoxycarbonyl-1-piperidyl)ethyl]-1-naphthyl]ethynyl]piperidine-1-carboxylate (8, 1 g, 1.76 mmol, 83% yield) as a thick off-white solid. LC-MS (ES⁺): m/z 533.4 [M+H]⁺.

Step-5:

To a well-stirred solution of a mixture of tert-butyl 4-[2-[5-[1-(4-isopropoxycarbonyl-1-piperidyl)ethyl]-1-naphthyl]ethynyl]piperidine-1-carboxylate (8, 1 g, 1.76 mmol) in 1:1:1 THE (10 mL):methanol (10 mL):water (10 mL) was added lithium hydroxide, monohydrate (740.47 mg, 17.65 mmol) at 0° C. The reaction mixture was stirred for 12 hours at 30° C. while the progress of the reaction was monitored by TLC (100% EtOAc in Petroleum Ether). Upon completion of the reaction, the reaction mixture was concentrated under vacuum, diluted with water (50 mL), and extracted with MTBE (2×150 ml). The aqueous phase was acidified with citric acid solution (pH 4) and extracted with EtOAc (2×250 mL). The organic layer was concentrated under reduced pressure to give 1-[1-[5-[2-(1-tert-butoxycarbonyl-4-piperidyl)ethynyl]-1-naphthyl]ethyl]piperidine-4-carboxylic acid (9, 900 mg, 1.74 mmol, 99% yield) as an off-white solid. LC-MS (ES⁺): m/z 491.1 [M+H]⁺.

Step-6:

To a 10 mL single-neck round-bottom flask containing a well-stirred solution of 1-[1-[5-[2-(1-tert-butoxycarbonyl-4-piperidyl)ethynyl]-1-naphthyl]ethyl]piperidine-4-carboxylic acid (9, 900 mg, 1.74 mmol) in anhydrous DMF (10 mL) were added N,N-diisopropylethylamine (1.13 g, 8.71 mmol, 1.52 mL) and HATU (993.91 mg, 2.61 mmol) followed by (3-fluorophenyl)methanamine (10, 327.12 mg, 2.61 mmol, 297.38 μL) at ambient temperature under nitrogen atmosphere. The contents were stirred at ambient temperature for 2 hours. After completion of the reaction, the reaction mixture was poured into water (50 mL) and extracted with EtOAc (2×250 mL). The combined organic layers were washed with brine (50 mL), dried over anhydrous Na₂SO₄, and filtered. The filtrate was concentrated under reduced pressure to give the crude product, which was purified by Biotage® Isolera (230-400 mesh silica-gel, 0-100% ethyl acetate/petroleumPetroleum ether). The product was further purified by reverse phase chromatography (Biotage Cis 120 g SNAP, with the mobile phase: Mobile Phase A: 0.1% Ammonium bicarbonate in water; Mobile phase B: Acetonitrile; Flow rate: 15 mL/min) to afford tert-butyl 4-[2-[5-[1-[4-[(3-fluorophenyl)methylcarbamoyl]-1-piperidyl]ethyl]-1-naphthyl]ethynyl]piperidine-1-carboxylate (11, 400 mg, 668.56 μmol, 38% yield) as an off-white solid. LC-MS (ES⁺): m/z 598.3 [M+H]⁺.

Step-7:

To a 100 mL single-neck round-bottom flask containing a well-stirred solution of tert-butyl 4-[2-[5-[1-[4-[(3-fluorophenyl)methylcarbamoyl]-1-piperidyl]ethyl]-1-naphthyl]ethynyl]piperidine-1-carboxylate (11, 390 mg, 651.85 μmol) in DCM (4 mL) was added 4 M hydrogen chloride in 1,4-dioxane, 99% (21.9 mL, 87.69 mmol) at 0° C. The reaction mixture was stirred at ambient temperature for 2 hours. Upon completion of the reaction, the reaction mixture was concentrated under reduced pressure to give a residue, which was washed with MTBE (2×200 mL) and acetonitrile (50 ml) and dried to give N-[(3-fluorophenyl)methyl]-1-[1-[5-[2-(4-piperidyl)ethynyl]-1-naphthyl]ethyl]piperidine-4-carboxamide dihydrochloride (12, 370 mg, 639.66 μmol, 98% yield) as a white solid. LCMS (ES⁺): m/z 498.3 [M+H]⁺.

### Example 75

### Synthesis of 1-[1-[4-[2-[1-[2-[4-[1-(2,6-dioxo-3-piperidyl)-2-oxo-benzo[cd]indol-6-yl]-1-piperidyl]acetyl]-4-piperidyl]ethynyl]-1-naphthyl]ethyl]-N-[(3-fluorophenyl)methyl]piperidine-4-carboxamide (Compound 102)

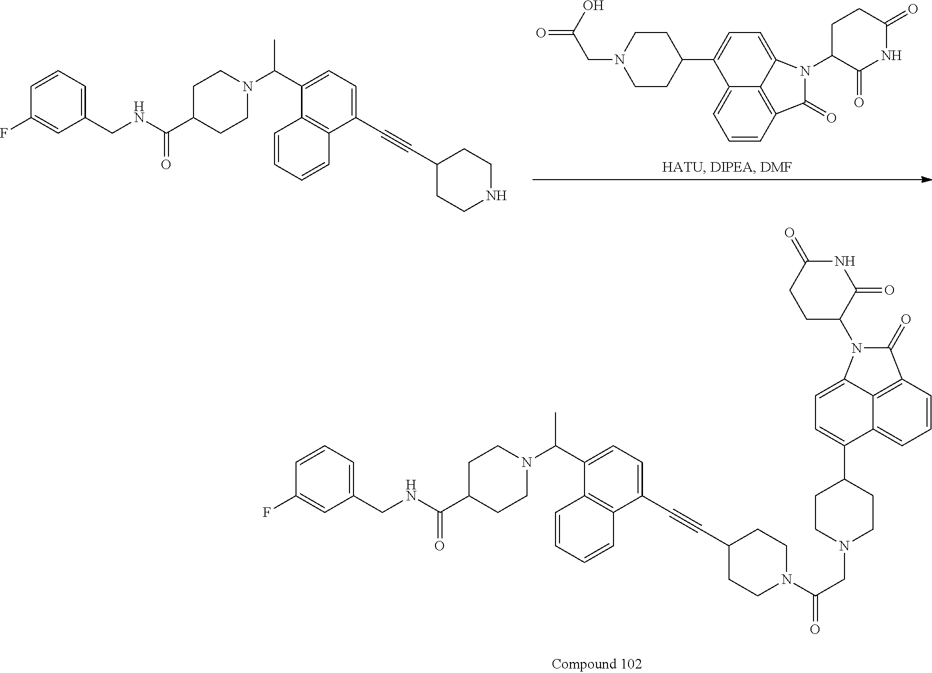 [SMILES: CC(c1ccc(C#CC2CCNCC2)c2ccccc12)N1CCC(C(=O)NCc2cccc(F)c2)CC1 ; O=C(O)CN1CCC(c2ccc3c4c(cccc24)C(=O)N3C2CCC(=O)NC2=O)CC1 ; CC(c1ccc(C#CC2CCN(C(=O)CN3CCC(c4ccc5c6c(cccc46)C(=O)N5C4CCC(=O)NC4=O)CC3)CC2)c2ccccc12)N1CCC(C(=O)NCc2cccc(F)c2)CC1]

To a stirred solution of N-[(3-fluorophenyl)methyl]-1-[1-[4-[2-(4-piperidyl)ethynyl]-1-naphthyl]ethyl]piperidine-4-carboxamide (30 mg, 51.35 μmol) and 2-[4-[1-(2,6-dioxo-3-piperidyl)-2-oxo-benzo[cd]indol-6-yl]-1-piperidyl]acetic acid (23.51 mg, 51.35 μmol) in DMF (0.5 mL) was added DIPEA (5 eq) and HATU (2.0 eq) and the reaction mixture was stirred for 5 hours at room temperature. Upon completion of the reaction, the reaction mixture was diluted with ice-cold water (10 ml) and the solid precipitation was filtered and dried under vacuum. The crude product was purified by Prep-HPLC (NH₄OAc method) to afford 1-[1-[4-[2-[1-[2-[4-[1-(2,6-dioxo-3-piperidyl)-2-oxo-benzo[cd]indol-6-yl]-1-piperidyl]acetyl]-4-piperidyl]ethynyl]-1-naphthyl]ethyl]-N-[(3-fluorophenyl)methyl]piperidine-4-carboxamide (15 mg, 15.05 μmol, 29% yield). LC-MS (ES⁺): m/z 901.5 [M+H]⁺. ¹H NMR (400 MHz, DMSO-d6) δ 11.11 (s, 1H), 8.49 (d, J=7.1 Hz, 1H), 8.43 (d, J=8.4 Hz, 1H), 8.37-8.20 (m, 2H), 8.09 (d, J=7.0 Hz, 1H), 7.85 (dd, J=8.3, 7.0 Hz, 1H), 7.75-7.42 (m, 4H), 7.45-7.21 (m, 2H), 7.13-6.82 (m, 4H), 5.40 (d, J=12.1 Hz, 1H), 4.25 (d, J=6.0 Hz, 2H), 4.21-4.07 (m, 1H), 4.05-3.80 (m, 2H), 3.62-3.38 (m, 6H), 3.30-3.18 (m, 1H), 3.21-2.57 (m, 6H), 2.38-2.26 (m, 2H), 2.24-1.46 (m, 15H), 1.38 (d, J=6.5 Hz, 3H).

Compound 103, Compound 104, and Compound 105 were prepared following the synthesis of Compound 102.

### Example 76

### 1-[1-[4-[2-[1-[2-[4-[1-(2,6-dioxo-3-piperidyl)-2-oxo-benzo[cd]indol-5-yl]-1-piperidyl]acetyl]-4-piperidyl]ethynyl]-1-naphthyl]ethyl]-N-[(3-fluorophenyl)methyl]piperidine-4-carboxamide (Compound 103)

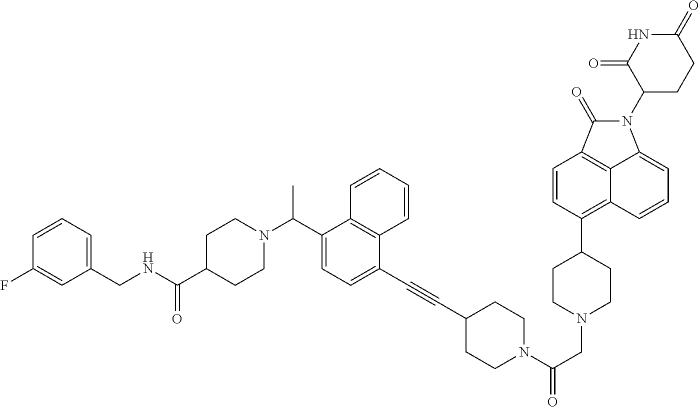 [SMILES: CC(c1ccc(C#CC2CCN(C(=O)CN3CCC(c4ccc5c6c(cccc46)N(C4CCC(=O)NC4=O)C5=O)CC3)CC2)c2ccccc12)N1CCC(C(=O)NCc2cccc(F)c2)CC1]

¹H NMR (400 MHz, DMSO-d6) δ 11.11 (s, 1H), 8.48 (d, J=7.8 Hz, 1H), 8.35-8.19 (m, 2H), 8.00 (d, J=7.3 Hz, 1H), 7.84 (d, J=8.7 Hz, 1H), 7.70 (d, J=7.4 Hz, 1H), 7.62 (d, J=7.5 Hz, 1H), 7.59-7.42 (m, 4H), 7.37-7.27 (m, 1H), 7.15 (d, J=7.2 Hz, 1H), 7.09-6.84 (m, 4H), 5.44 (dd, J=12.9, 5.4 Hz, 1H), 4.24 (d, J=5.9 Hz, 2H), 4.21-4.09 (m, 1H), 4.06-3.76 (m, 2H), 3.56-3.36 (m, 1H), 3.30-3.16 (m, 2H), 3.17-3.08 (m, 1H), 3.02 (d, J=10.1 Hz, 3H), 2.96-2.86 (m, 1H), 2.86-2.54 (m, 4H), 2.41-2.23 (m, 2H), 2.22-1.42 (m, 15H), 1.38 (d, J=6.5 Hz, 3H). LC-MS (ES⁺): m/z 901.4 [M+H]⁺.

### Example 77

### 1-[1-[5-[2-[1-[2-[4-[1-(2,6-dioxo-3-piperidyl)-2-oxo-benzo[cd]indol-6-yl]-1-piperidyl]acetyl]-4-piperidyl]ethynyl]-1-naphthyl]ethyl]-N-[(3-fluorophenyl)methyl]piperidine-4-carboxamide (Compound 104)

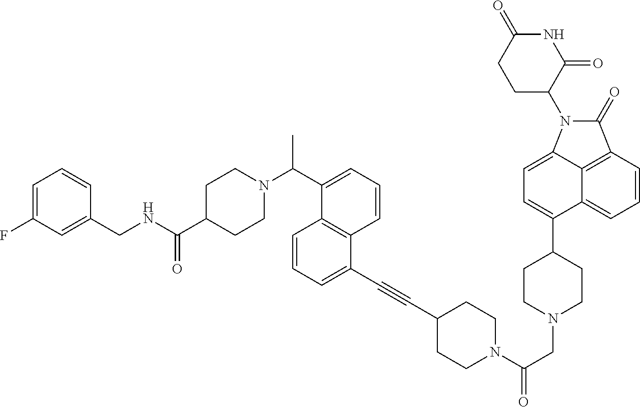 [SMILES: CC(c1cccc2c(C#CC3CCN(C(=O)CN4CCC(c5ccc6c7c(cccc57)C(=O)N6C5CCC(=O)NC5=O)CC4)CC3)cccc12)N1CCC(C(=O)NCc2cccc(F)c2)CC1]

¹H NMR (400 MHz, DMSO-d6) δ 11.13 (s, 1H), 8.47 (dd, J=23.9, 8.6 Hz, 3H), 8.30 (t, J=6.0 Hz, 1H), 8.18 (d, J=8.2 Hz, 1H), 8.09 (t, J=7.1 Hz, 1H), 7.86 (t, J=7.6 Hz, 1H), 7.65 (d, J=7.0 Hz, 1H), 7.62-7.42 (m, 4H), 7.35 (ddd, J=14.0, 9.1, 6.7 Hz, 3H), 7.04 (ddd, J=19.1, 13.4, 8.7 Hz, 5H), 5.44 (d, J=12.6 Hz, 1H), 4.25 (d, J=6.0 Hz, 3H), 4.21-4.08 (m, 1H), 3.95 (s, 2H), 3.60-3.35 (m, 2H), 2.98 (d, J=18.8 Hz, 4H), 2.87-2.57 (m, 4H), 2.36-2.20 (m, 3H), 2.22-1.44 (m, 12H), 1.40 (d, J=6.4 Hz, 4H). LC-MS (ES⁺): m/z 901.4 [M+H]⁺.

### Example 78

### 1-[1-[5-[2-[1-[2-[4-[1-(2,6-dioxo-3-piperidyl)-2-oxo-benzo[cd]indol-5-yl]-1-piperidyl]acetyl]-4-piperidyl]ethynyl]-1-naphthyl]ethyl]-N-[(3-fluorophenyl)methyl]piperidine-4-carboxamide (Compound 105)

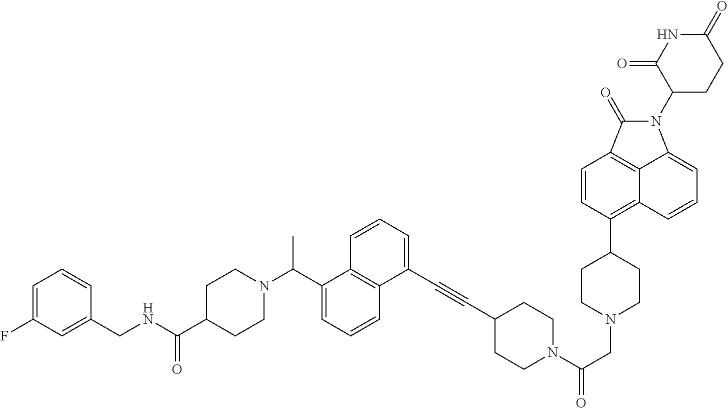 [SMILES: CC(c1cccc2c(C#CC3CCN(C(=O)CN4CCC(c5ccc6c7c(cccc57)N(C5CCC(=O)NC5=O)C6=O)CC4)CC3)cccc12)N1CCC(C(=O)NCc2cccc(F)c2)CC1]

¹H NMR (400 MHz, DMSO-d6) δ 11.11 (s, 1H), 8.49 (d, J=8.7 Hz, 1H), 8.29 (t, J=6.0 Hz, 1H), 8.18 (d, J=8.3 Hz, 1H), 8.00 (d, J=7.3 Hz, 1H), 7.85 (d, J=8.8 Hz, 1H), 7.70 (d, J=7.4 Hz, 1H), 7.64 (d, J=7.0 Hz, 1H), 7.60-7.42 (m, 3H), 7.38-7.25 (m, 1H), 7.15 (d, J=7.2 Hz, 1H), 7.09-6.88 (m, 3H), 5.44 (dd, J=12.9, 5.4 Hz, 1H), 4.24 (d, J=5.9 Hz, 2H), 4.21-4.08 (m, 1H), 3.96 (d, J=12.8 Hz, 2H), 3.38 (s, 3H), 3.28 (s, 2H), 3.02 (s, 3H), 2.83-2.69 (m, 2H), 2.70-2.58 (m, 3H), 2.39-2.23 (m, 2H), 2.24-1.44 (m, 14H), 1.39 (d, J=6.5 Hz, 3H). LC-MS (ES⁺): m/z 901.4 [M+H]⁺.

All publications and patent applications cited in this specification are herein incorporated by reference as if each individual publication or patent application were specifically and individually indicated to be incorporated by reference.

Although the foregoing invention has been described in some detail by way of illustration and example for the purposes of clarity of understanding, it will be readily apparent to one of ordinary skill in the art in light of the teaching of this invention that certain changes and modifications may be made thereto without departing form the spirt or scope of the invention as defined in the claims and embodiments.

We claim:

1. A compound of Formula: 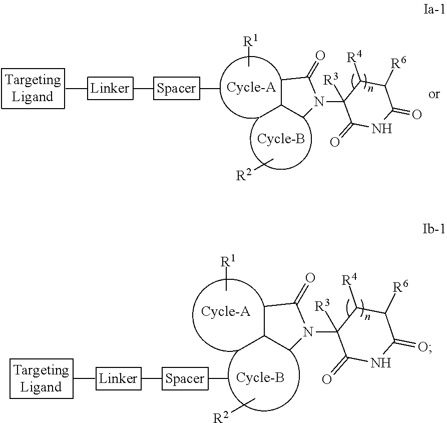
or a pharmaceutically acceptable salt thereof,
wherein:
n is 0, 1, or 2;
Cycle-A is a fused ring selected from the group consisting of phenyl, 5- or 6-membered heteroaryl, 5- to 8-membered heterocycle, 5- to 8-membered cycloalkyl, or 5- to 8-membered cycloalkenyl;
Cycle-B is a fused ring selected from the group consisting of phenyl, 5- or 6-membered heteroaryl, 5- to 8-membered heterocycle, 5- to 8-membered cycloalkyl, or 5- to 8-membered cycloalkenyl;
R¹ and R² are independently at each instance selected from the group consisting of hydrogen, alkyl, halogen, haloalkyl, —OR¹⁰, —SR¹⁰, —S(O)R¹², —SO₂R¹², —NR¹⁰R¹¹, cyano, nitro, heteroaryl, aryl, and heterocycle; or alternatively, if allowed by valence and stability, R¹ or R² may be a divalent moiety such as ═O, ═S, or ═NR⁴¹; and wherein an R¹ group may optionally be combined with another R¹ group or an R² group to form a fused cycle or bicycle which may bridge Cycle-A and Cycle-B, as appropriate;
R³ is hydrogen, alkyl, halogen, or haloalkyl;
or R³ and R⁶ are combined to form a 1 or 2 carbon attachment;
or R³ and R⁴ are combined to form a 1, 2, 3, or 4 carbon attachment;
or R³ and an R⁴ group adjacent to R³ are combined to form a double bond;
R¹⁰ and R¹¹ are independently selected from the group consisting of hydrogen, alkyl, haloalkyl, heterocycle, aryl, heteroaryl, —C(O)R¹², —S(O)R¹², and —SO₂R¹²;
each R¹² is independently selected from the group consisting of hydrogen, alkyl, haloalkyl, heterocycle, aryl, heteroaryl, —NR¹³R¹⁴, and OR¹³;
each instance of R¹³ and R¹⁴ is independently selected from the group consisting of hydrogen, alkyl, and haloalkyl;
Spacer is a bivalent connecting moiety of the structure: 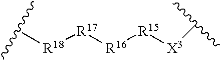
X³ is a bivalent moiety selected from the group consisting of a bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂— and —S—; or can be arylalkyl, heterocycloalkyl and heteroarylalkyl each of which heterocycle, aryl, heteroaryl, and bicycle may be substituted with 1, 2, 3, or 4 substituents independently selected from R⁴⁰;
R¹⁵, R¹⁶, R¹⁷, and R¹⁸ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴¹)—, —P(O)(OR²⁶)O—, —P(O)(OR²⁶)—, bicycle, alkene, alkyne, haloalkyl, alkoxy, aryl, heterocycle, heteroaryl, lactic acid, glycolic acid, arylalkyl, heterocycloalkyl, and heteroarylalkyl; each of which is optionally substituted with 1, 2, 3, or 4 substituents independently selected from R⁴⁰;
R²⁶ is independently at each occurrence selected from the group consisting of hydrogen, alkyl, arylalkyl, heteroarylalkyl, alkene, alkyne, aryl, heteroaryl, and heterocycle;
R²⁷ is independently at each occurrence selected from the group consisting of hydrogen, alkyl, heterocycle, aryl, heteroaryl, —C(O)(aryl or heteroaryl), —C(O)O(aryl or heteroaryl), alkene, and alkyne;
R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(alkyl), —N(alkyl)₂, —NHSO₂(alkyl), —N(alkyl)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aryl, heteroaryl, heterocycle, and cycloalkyl;
R⁴¹ is aryl, heteroaryl, or hydrogen;
Linker is a bivalent linking group of formula: 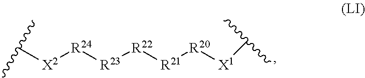
wherein:
X¹ and X² are independently at each occurrence selected from a bond, heterocycle, aryl, heteroaryl, bicycle, —NR²⁷—, —CR⁴⁰R⁴¹—, —O—, —C(O)—, —C(NR²⁷)—, —C(S)—, —S(O)—, —S(O)₂— and —S—;
each of which heterocycle, aryl, heteroaryl, and bicycle is substituted with 1, 2, 3, or 4 substituents independently selected from R⁴⁰;
R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, —C(R⁴⁰R⁴⁰)—, —P(O)(OR²⁶)O—, —P(O)(OR²⁶)—, bicycle, alkene, alkyne, haloalkyl, alkoxy, aryl, heterocycle, heteroaryl, lactic acid, glycolic acid, and carbocycle; each of which is optionally substituted with 1, 2, 3, or 4 substituents independently selected from R⁴⁰;
Targeting Ligand is a ligand that binds to a Target Protein that mediates a cancer; and
Target Protein is selected from the group consisting of: ADAR, AKT1, ALK, AXL, ABL1, ABL2, AKT2, AP1, AP2, ARID1B, ASH1L, ATAD2, aurora kinase, androgen receptor, ATF2, BMX, BCR-ABL, Bcl-2, Bcl-XL, BCL6, BAZ2A, BAZ2B, BRD4, BRD9, BRPF1, CSF1R, CECR2, CBP, CREBBP, CTNNB1, cyclin dependent kinase, DDR1, DOT1L, ERBB2, ERBB3, ERBB4, EPHA2, EPHA3, EPHA4, EPHA7, EPHB4, EZH2, EED, EHMT1, EHMT2, ERK1, ERK2, estrogen receptor, EGFR, FGFR1, FGFR2, FGFR3, FGFR4, FLT3, FYN, factor Xa, GSG2, HDAC6, HDAC7, HDM2, HCK, hsp90, Her3, IGF1R, IKZF1, IKZF2, IKZF3, IKZF4, INSR, IDO1, IDH1, ITK, KDM4, KDM5, KDM6, KMT5A, KIT, kallikrein 7, KRAS, LSD1, LYN, mPGES-1, MERTK, MEK1, MDM2, MDM4, MEN1, MTH1, MST1R, NFE2L2, NSD2, NTRK1, NTRK2, NTRK3, p63, P300, PCAF, PAK1, PAK4, PIK3CA, RET, RIT1, SETD2, SETD7, SETD8, SETDB1, TAF1, mTORC1, mTORC2, TANK1, WRN, WDR5, and YES1.

2. The compound of claim 1, wherein the Targeting Ligand is selected from a structure described in FIG. 1A-FIG. 70, optionally substituted with 1, 2, 3, or 4 R⁴⁰ substituents.

3. The compound of claim 1, wherein Cycle-A is phenyl.

4. The compound of claim 1, wherein Cycle-B is phenyl.

5. The compound of claim 1, wherein R² group is alkyl, halogen, haloalkyl, heteroaryl, aryl, heterocycle, —OR¹⁰, —SR¹⁰, —S(O)R¹², —SO₂R¹², or —NR¹⁰R¹¹.

6. The compound of claim 1, wherein R² is hydrogen.

7. The compound of claim 1, wherein R¹ is alkyl, halogen, haloalkyl, heteroaryl, aryl, heterocycle —OR¹⁰, —SR¹⁰, —S(O)R¹², —SO₂R¹², or —NR¹⁰R¹¹.

8. The compound of claim 1, wherein R¹ is hydrogen.

9. The compound of claim 1, wherein X³ is bond, heterocycle, NR²⁷, or C(O).

10. The compound of claim 1, wherein R¹⁵, R¹⁶, R¹⁷, and R¹⁸ are independently selected from the group consisting of bond, CH₂, heterocycle, aryl, and bicycle.

11. The compound of claim 1, wherein the compound is of Formula: 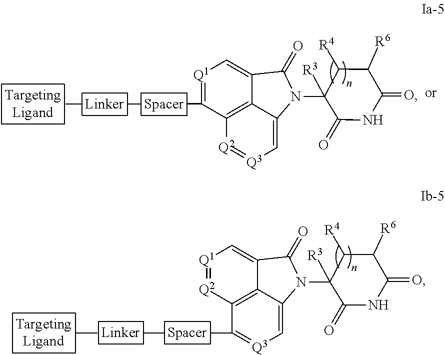
wherein Q¹, Q², and Q³ are independently selected from the group consisting of CH, CR¹, and N and wherein only one of Q¹, Q², and Q³ is CR¹;
or a pharmaceutically acceptable salt thereof.

12. The compound of claim 11, wherein R³, R⁴, and R⁶ are hydrogen.

13. The compound of claim 12, wherein n is 1.

14. The compound of claim 1, wherein the Spacer is selected from the group consisting of:

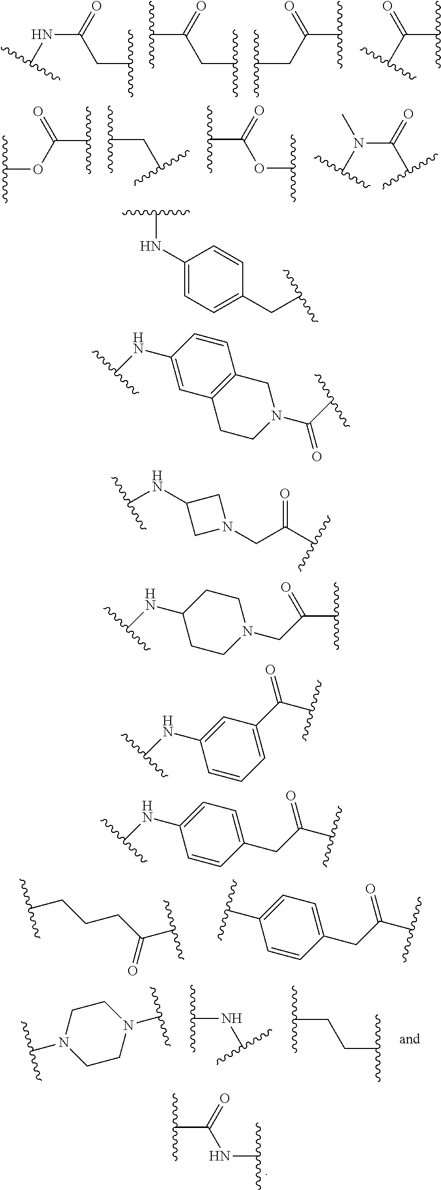

15. The compound of claim 1, wherein the Target Protein is selected from the group consisting of: ABL1, ABL2, ALK, AXL, BMX, CSF1R, DDR1, EGFR, FGFR1, FGFR2, FGFR3, FGFR4, FYN, GSG2, HCK, INSR, ITK, MEN1, MTH1, MST1R, NFE2L2, NTRK1, NTRK2, NTRK3, RET, mTORC1, mTORC2, TANK1, WRN, WDR5, and YES1.

16. The compound of claim 1, wherein the Target Protein is selected from the group consisting of: AKT2, AP1, AP2, ARID1B, aurora kinase, BCR-ABL, DOT1L, ERBB2, ERBB3, ERBB4, EPHA2, EPHA3, EPHA4, EPHA7, EPHB4, EZH2, EED, EHMT1, EHMT2, KMT5A, KIT, kallikrein 7, MDM2, MDM4, NSD2, PAK1, PAK4, PIK3CA, RIT1,

17. The compound of claim 1, wherein the Target Protein is a cyclin dependent kinase.

18. The compound of claim 1, wherein the Target Protein is selected from the group consisting of: ADAR, AKT1, ATF2, ASH1L, ATAD2, BAZ2A, BAZ2B, BRD4, BRD9, BRPF1, CECR2, CREBBP, ERK1, ERK2, FLT3, factor Xa, IGF1R, IDO1, IDH1, KDM4, KDM5, KDM6, PCAF, and TAF1.

19. The compound of claim 1, wherein the Target Protein is an androgen receptor or an estrogen receptor.

20. The compound of claim 1, wherein the Target Protein is Bcl-2, Bcl-XL, BCL6, CBP, CTNNB1, p63, P300, IKZF1, IKZF2, IKZF3, IKZF4, HDAC6, HDAC7, HDM2, hsp90, Her3, KRAS, LSD1, LYN, mPGES-1, MERTK, MEK1, SETD2, SETD7, SETD8, or SETDB1.

21. A compound selected from the group consisting of 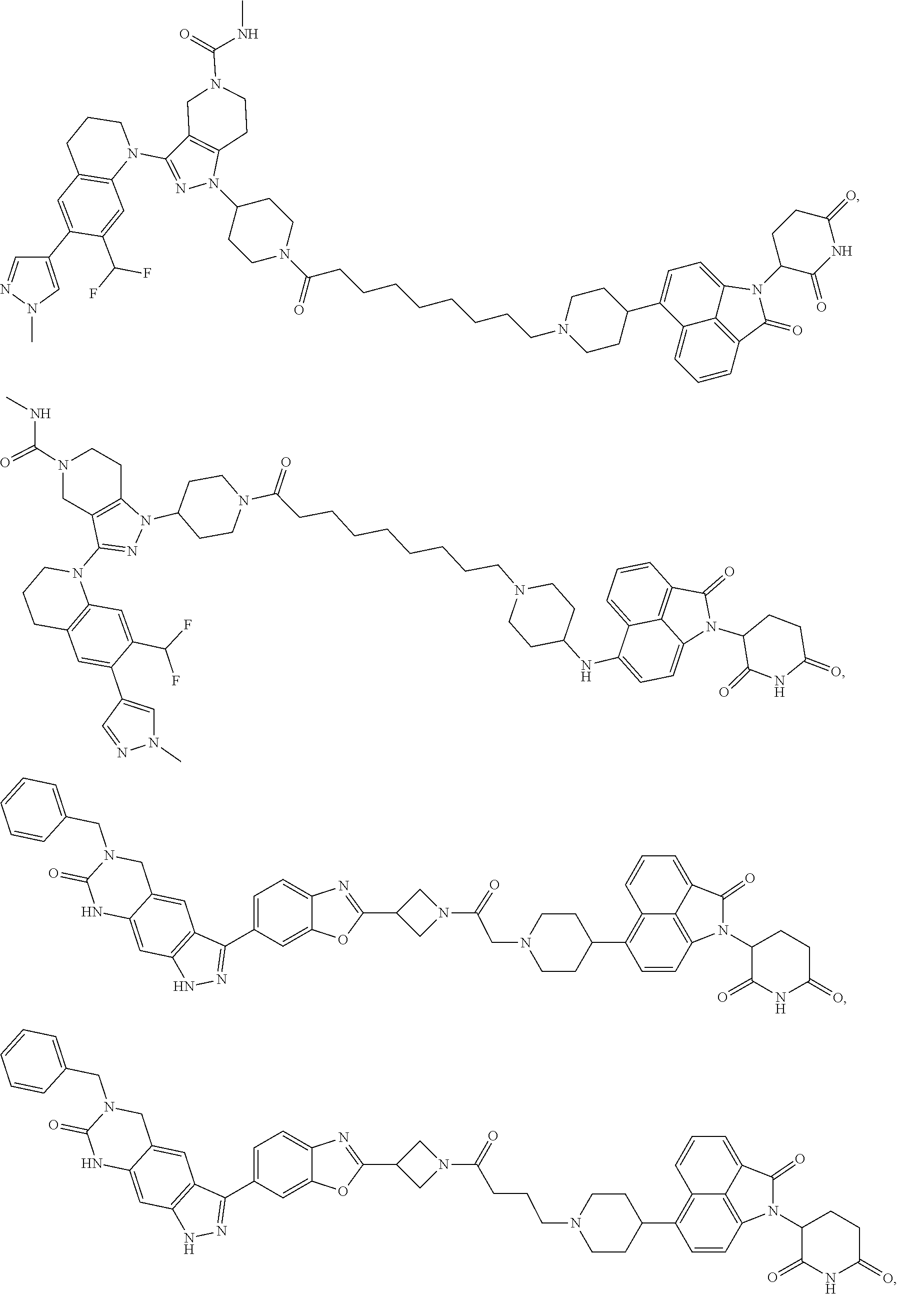 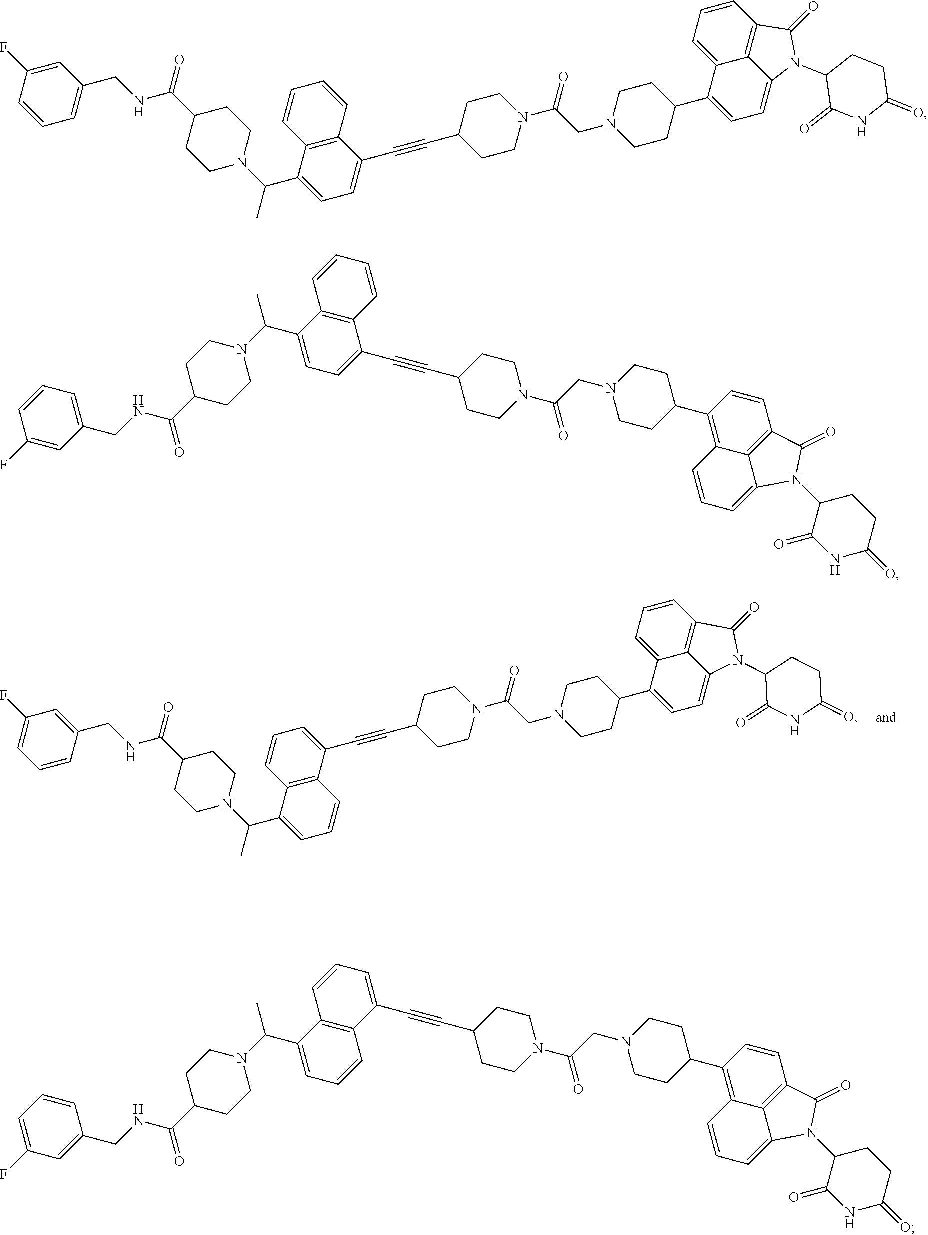 [SMILES: CC(c1ccc(C#CC2CCN(C(=O)CN3CCC(c4ccc5c6c(cccc46)C(=O)N5C4CCC(=O)NC4=O)CC3)CC2)c2ccccc12)N1CCC(C(=O)NCc2cccc(F)c2)CC1 ; CC(c1ccc(C#CC2CCN(C(=O)CN3CCC(c4ccc5c6c(cccc46)N(C4CCC(=O)NC4=O)C5=O)CC3)CC2)c2ccccc12)N1CCC(C(=O)NCc2cccc(F)c2)CC1 ; CC(c1cccc2c(C#CC3CCN(C(=O)CN4CCC(c5ccc6c7c(cccc57)C(=O)N6C5CCC(=O)NC5=O)CC4)CC3)cccc12)N1CCC(C(=O)NCc2cccc(F)c2)CC1 ; CC(c1cccc2c(C#CC3CCN(C(=O)CN4CCC(c5ccc6c7c(cccc57)N(C5CCC(=O)NC5=O)C6=O)CC4)CC3)cccc12)N1CCC(C(=O)NCc2cccc(F)c2)CC1]
or a pharmaceutically acceptable salt thereof.

22. A pharmaceutical composition comprising a compound of claim 1 or a pharmaceutically acceptable salt thereof and a pharmaceutically acceptable carrier.

23. A method of treating a cancer that is mediated by the Target Protein in a human in need thereof comprising administering an effective amount of a compound of claim 1 or a pharmaceutically acceptable salt thereof.
